SEQUENCE LISTING (4 sequences)

SEQ ID NO: 1 - protein, 1525 aa, Thermus aquaticus
  MISC_FEATURE (1247)..(1247): Xaa can be any amino acid.
MKKEVRKVRIALASPEKIRSWSYGEVEKPETINYRTLKPERDGLFDERIFGPIKDYECACGKYKRQRFEGKVCERCGVEVTRSIVRRYRMGHIELATPAAHIWFVKDVPSKIGTLLDLFATELEQVLYFNKYIVLDPKGAVLDGVPVEKRQLLTDEEYRELRYGKQETYPLPAGVDALVKDGEEVVKGQELAPGVVSRMDGVGSLPLPRRVRVDYLRKERAALRIPLSAWVEKEPYRPGEVLAELSEPYLFRAEESGVVELKDLAEGHLIYLRQEEEVVARYFLPAGMTPLVVEGEIVEVGQPLAEGKGLLRLPRHMTAKEVEAEEEGDSVHLTLFLEWTEPKDYKVAPHMNVIVPEGAKVQAGEKIVAAIDPEEEVIAQAEGVVHLHEPASILVVKARVYPFEDDVEVTTGDRVAPGDVLADGGKVKSEIYGRVEVDLVRNVVRVVESYDIDARMGAEAIQELLKELDLEKLERELLEEMKHPSRARRAKARKRLEVVRAFLDSGNRPEWMILEAVPVLPPDLRPMVQVDGGRFATSDLNDLYRRLINRNNRLKKLLAQGAPEIIIRNEKRMLQEAVDAVIDNGRRGSPVTNPGSERPLRSLTDILSGKQGRFRQNLLGKRVDYSGRSVIVVGPQLKLHQCGLPKRMALELFKPFLLKKMEEKAFAPNVKAARRMLERQRDIKDEVWDALEEVIHGKVVLLNRAPTLHRLGIQAFQPVLVEGQSIQLHPLVCEAFNADFDGDQMAVHVPLSSFAQAEARIQMLSAHNLLSPASGEPLAKPSRDIILGLYYITQVRKEKKGAGMAFATPEEALAAYERGEVALNAPIVVAGRETSVGRLKFVFANPDEALLAVAHGLLDLQDVVTVRYLGRRLETNPGRILFARIVGEAVGDEKVAQELIQMDVPQEKNSLKDLVYQAFLRLGMEKTARLLDALKYYGFTLSTTSGIITIGIDDAVIPEEKQRYLEEADRKLRQIEQAYEMGFLTDRERYDQVIQLWTETTEKVTQAVFNNFEENYPFNPLYVMAQSGARGNPQQIRQLCGMRGLMQKPSGETFEVPVRSSFREGLTVLEYFISSHGARKGGADTALRTADSGYLTRKLVDVAHEIVVREADCGTTKYISVPLFQMDEVTRTLRLRKRSDIESGLYGRVLAREVEALGRRLEEGRYLSLEDVHFLIKAAEAGEVREVPVRSPLTCQTRYGVCQKCYGYDLSMARPVSIGEAVGVVAAESIGEPGTQLTMRTFHTGGXAVGTDITQGLPRVIELFEARRPKAKAVISEIDGVVRIEEGEDRLSVFVESEGFSKEYKLPKDARLLVKDGDYVEAGQPLTRGAIDPHQLLEAKGPEAVERYLVDEIQKVYRAQGVKLHDKHIEIVVRQMLKYVEVTDPGDSPLLEGQVLEKWDVEALNERLIAEGKVPVAWKPLLMGVTKSALSTKSWLSAASFQNTTHVLTEAAIAGKKDELIGLKENVILGRLIPAGTGSDFVRFTQVVDQRTLKAIEEARKEAVEAKEKEAPRRPVRREQPGKGL

SEQ ID NO: 2 - protein, 1119 aa, Thermus aquaticus
  MISC_FEATURE (695)..(696): Xaa can be any amino acid
MKIKRFGRIREVIPLPPLTEIQVESYKKALQADVPPEKRENVGIQAAFKETFPIEEGDKGKGGLVLDFLEYRIGDPPFSQDECREKDLTYQAPLYARLQLIHKDTGLIKEDEVFLGHLPLMTEDGSFIINGADRVIVSQIHRSPGVYFTPDPARPGRYIASIIPLPKRGPWIDLEVEASGVVTMKVNKRKFPLVLLLRVLGYDQETLVRELSAYGDLVQGLLDEAVLAMRPEEAMVRLFTLLRPGDPPKKDKALAYLFGLLADPKRYDLGEAGRYKAEEKLGVGLSGRTLVRFEDGEFKDEVFLPTLRYLFALTAGVPGHEVDDIDHLGNRRIRTVGELMADQFRVGLARLARGVRERMVMGSPDTLTPAKLVNSRPLEAALREFFSRSQLSQFKDETNPLSSLRHKRRISALGPGGLTRERAGFDVRDVHRTHYGRICPVETPEGANIGLITSLAAYARVDALGFIRTPYRRVKNGVVTEEVVYMTASEEDRYTIAQANTPLEGDRIATDRVVARRRGEPVIVAPEEVEFMDVSPKQVFSLNTNLIPFLEHDDANRALMGSNMQTQAVPLIRAQAPVVMTGLEERVVRDSLAALYAEEDGEVVKVDGTRIAVRYEDGRLVEHPLRRYARSNQGTAFDQRPRVRVGQRVKKGDLLADGPASEEGFLALGQNVLVAIMPFDGYNFEDAIVISEELXXRDFYTSIHIERYEIEARDTKLGPERITRDIPHLSEAALRDLDEEGIVRIGAEVKPGDILVGRTSFKGEQEPSPEERLLRSIFGEKARDVKDTSLRVPPGEGGIVVGRLRLRRGDPGVELKPGVREVVRVFVAQKRKLQVGDKLANRHGNKGVVAKILPVEDMPHLPDGTPVDVILNPLGVPSRMNLGQILETHLGLAGYFLGQRYISPVFDGATEPEIKELLAEAFNLYFGKRQGEGFGVDKREKEVLARAEKLGLVSPGKSPEEQLKELFDLGKVVLYDGRTGEPFEGPIVVGQMFIMKLYHMVEDKMHARSTGPYSLITQQPLGGKAQFGGQRFGEMEVWALEAYGAAHTLQEMLTIKSDDIEGRNAAYQAIIKGEDVPEPSVPESFRVLVKELQALALDVQTLDEKDNPVDIFEGLASKR

SEQ ID NO: 3 - protein, 313 aa, Thermus aquaticus
MLESKLKAPVFTATTQGDHYGEFVLEPLERGFGVTLGNPLRRILLSSIPGTAVTSVYIEDVLHEFSTIPGVKEDVVEIILNLKELVVRFLDPRWRTTLILRAEGPKEVRAVDFTPSADVEIMNPDLHIATLEEGGKLYMEVRVDRGVGYVPAERHGIKDRINAIPVDAIFSPVRRVAFQVEDTRLGQRTDLDKLTLRIWTDGSVTPLEALNQAVAILKEHLNYFANPEASLLPTPEVSKGEKRESAEEDLDLPLEELGLSTRVLHSLKEEGIESVRALLALNLKDLRNIPGIGERSLEEIRQALAKKGFTLKE

SEQ ID NO: 4 - protein, 99 aa, Thermus aquaticus
MAEPGIDKLFGMVDSKYRLTVVVAKRAQQLLRHRFKNTVLEPEERPKMRTLEGLYDDPNAVTWAMKELLTGRLFFGENLVPEDRLQKEMERLYPTEEEA

(19) United States
(12) Patent Application Publication
(10) Pub. No.: US 2004/0048350 A1
(43) Pub. Date: Mar. 11, 2004

(54) Crystal of bacterial core RNA polymerase with rifampicin and methods of use thereof

(76) Inventors: Seth A. Darst, New York, NY (US); Elizabeth Campbell, New York, NY (US)

Correspondence Address: FOLEY HOAG LLP, PATENT GROUP, 155 SEAPORT BOULEVARD, BOSTON, MA, 02110, US

(21) Appl. No.: 09/802,755

(22) Filed: Mar. 9, 2001

(51) Int. Cl. C12N 9/22; G01N 33/53; G06G 7/48; G06G 7/58; G06F 19/00; G01N 33/48

(52) U.S. Cl. 435/199; 702/19; 703/11

(57) ABSTRACT

A detailed three-dimensional structure of rifampicin bound to a core bacterial RNA polymerase (Rif-RNAP) is provided. Crystals of the Rif-RNAP are also included in the invention. The present invention further provides procedures for identifying agents that can inhibit bacterial proliferation through the use of rational drug design predicated on the crystals and crystallographic data disclosed.

### GOVERNMENTAL SUPPORT

The research leading to the present invention was supported, at least in part, by grants from NIH, Grants GM 53759, GM 20470, GM 61898, GM 49242 and GM 30717. Accordingly, the Government may have certain rights in the invention.

### REFERENCE TO TABLE SUBMITTED ON COMPACT DISC

Two compact discs are included with the instant filing which contain identical material. The material on the compact disc is hereby incorporated by reference in its entirety under 37 CFR §1.77(b)(4). The compact discs contain a single file, dated Mar. 9, 2001, labeled RNAP_RIF_final.pdb which is an ASCII text file that is 1.46 MB (1,536,303 bytes), 1,540,096 bytes used. The compact discs contain the structural coordinates for the Rif-RNAP complex with the Thermus aquaticus core RNA polymerase which is also included in a hard copy as Table 2 in the Appendix, following the Sequence Listing.

### FIELD OF THE INVENTION

The present invention provides a crystal of a binding complex between rifampicin and a bacterial core RNA polymerase from Thermus aquaticus. The three-dimensional structural information is included in the invention. The present invention provides procedures for identifying agents that can inhibit bacterial cell growth through the use of rational drug design predicated on the crystallographic data.

### BACKGROUND OF THE INVENTION

RNA in all cellular organisms is synthesized by a complex molecular machine, the DNA-dependent RNA polymerase (RNAP). In its simplest bacterial form, the enzyme comprises at least 4 subunits with a total molecular mass of around 400 kDa. The eukaryotic enzymes comprise upwards of a dozen subunits with a total molecular mass of around 500 kDa. The essential core component of the RNAP (subunit composition α₂ββ′ω) is evolutionarily conserved from bacteria to man [Archambault and Friesen, Microbiological Reviews, 57:703-724 (1993)]. Sequence homologies point to structural and functional homologies, making the simpler bacterial RNAPs excellent model systems for understanding the multisubunit cellular RNAPs in general.

The basic elements of the transcription cycle were elucidated through study of the prokaryotic system. In this cycle, the RNAP, along with other factors, locates specific sequences called promoters within the double-stranded DNA, forms the open complex by melting a portion of the DNA surrounding the transcription start site, initiates the synthesis of an RNA chain, and elongates the RNA chain completely processively while translocating itself and the melted transcription bubble along the DNA template. Finally it releases itself and the completed transcript from the DNA when a specific termination signal is encountered. The current view is that the transcribing RNAP contains sites for binding the DNA template as well as forming and maintaining the transcription bubble, binding the RNA transcript, and binding the incoming nucleotide-triphosphate substrate.

From the initial indications of DNA-dependent RNAP activity from a number of systems, [Weiss and Gladstone, J. Am. Chem. Soc., 81:4118-4119 (1959)]; Hurwitz et al., Biochem. Biophys. Res. Commun., 3:15 (1960); Stevens, Biochem. Biophys. Res. Commun., 3:92 (1960); Huang et al., Biochem. Biophys. Res. Commun., 3:689 (1960); and Weiss and Nakamoto, J. Biol. Chem., 236:PC 19 (1961)], and the isolation of the RNAP enzyme from bacterial sources [Chamberlin and Berg, Proc. Natl. Acad. Sci. USA, 48:81-94 (1962)], a wealth of biochemical, biophysical, and genetic information has accumulated on RNAP and its complexes with nucleic acids and accessory factors. Nevertheless, the enzyme itself, in terms of its structure/function relationship, remains a black box. An essential step towards understanding the mechanism of transcription and its regulation is to determine three-dimensional structures of RNAP and its complexes with DNA, RNA, and regulatory factors [von Hippel et al., Annual reviews of Biochemistry, 53:389-446 (1984); Erie et al., Annual Review of Biophysics & Biomolecular Structure, 21:379-415 (1992); Sentenac et al., Transcriptional Regulation, in Cold Spring Harbor Laboratory 27-54, Cold Spring Harbor, eds. McKnight and Yamamoto (1992); Gross et al., Philosophical Transactions of the Royal Society of London—Series B:Biological Sciences, 351:475-482 (1996); and Nudler, J. Mol. Biol., 288:1-12 (1999)].

The key feature of low-resolution structures of bacterial and eukaryotic RNAPs, provided by electron crystallography, is a thumb-like projection surrounding a groove or channel that is an appropriate size for accommodating double-helical DNA [Darst et al., Nature, 340:730-732 (1989); Darst et al., Cell, 66:121-128 (1991); Schultz et al., EMBO J., 12:2601-2607 (1993); Polyakov et al., Cell, 83:365-373 (1995); Darst et al., J. Structural Biol., 124:115-122 (1998); and Darst et al., Cold Spring Harbor Symp. Quant. Biol., 63:269-276 (1998)].

Bacterial infections remain among the most common and deadly causes of human disease. Infectious diseases are the third leading cause of death in the United States and the leading cause of death worldwide [Binder et al., Science 284:1311-1313 (1999)]. More particularly, each year there are 8-10 million new cases of tuberculosis (TB). TB is the leading cause of death in adults by an infectious agent [Raviglioni et al., JAMA 273:220-226 (1995); Shinnick, Current Topics in Microbiol. Immunol., Springer-Verlag Berlin Heidelberg, New York (1996)] and is in near epidemic proportions in some parts of the world. Indeed, the World Health Organization declared TB to be a global public health emergency due to the rapid increase in multi-drug resistant strains of Mycobaterium tuberculosis [Raviglioni et al., JAMA 273:220-226 (1995)].

Rifampicin (Rif) [Sensi, Antibiot.Ann 1959-1960, 262-270 (1960); Sensi et al., Rev.Infect.Dis., 5 Supp.3:402-406 (1983)] is one of the most potent and broad-spectrum antibiotics against bacterial pathogens and is a key component of anti-TB therapy. The introduction of rifampicin in 1968 greatly shortened the duration of chemotherapy necessary for successful treatment. Rifampicin diffuses freely into tissues, living cells, and bacteria, making it extremely effective against intracellular pathogens like M. tuberculosis [Shinnick, Current Topics in Microbiol. Immunol., Springer-Verlag Berlin Heidelberg, New York (1996)]. However, bacteria develop resistance to rifampicin with high frequency, which has led the medical community in the United States to commit to a voluntary restriction of its use for treatment of TB or emergencies.

The bactericidal activity of rifampicin stems from its high-affinity binding to, and inhibition of, the bacterial DNA-dependent RNA polymerase [Hartmann et al., Biochim.Biophys. Acta 145:843-844 (1967)]. Mutations conferring rifampicin resistance (Rif^{R}) map almost exclusively to the rpoB gene (encoding the RNAP β subunit) in every organism tested, including E. coli [Ezekiel and Hutchins, Nature London 220:276-277(1968); Heil and Zillig, FEBS Lett. 11: 165-168 (1970); Wehrli et al., Biochem.Biophysic.Res. Comm., 32:284-288 (1968) and M. tuberculosis [Heep et al., Antimicrob.Agents Chemotherap.44: 1075-1077 (2000); Ramaswamy and Musser, Tubercle and Lung Disease 79:3-29 (1998)]. Comprehensive genetic analyses have provided molecular details of amino acid alterations in β subunit conferring Rif^{R} (see FIG. 1) [Jin and Gross, J.Molec.Biol, 202:45-58 1988; Lisitsyn et al., Bioorg Khim 10:127-128 (1984); Lisitsyn et al., Molec. Gen. Genet., 196:173-174 (1984); Ovchinnikov et al., Molec.Gen.Genet.190:344-348 (1983); Severinov et al., J.Biol.Chem., 268:14820-14825 (1993); Severinov et al., Molec.Gen.Genet., 244:120-126 (1994)].

Although, there was initial optimism in the middle of this century that diseases caused by bacteria would be quickly eradicated, it has become evident that the so-called “miracle drugs” are not sufficient to accomplish this task. Indeed, antibiotic resistant pathogenic strains of bacteria have become common-place, and bacterial resistance to the new variations of these drugs appears to be outpacing the ability of scientists to develop effective chemical analogs of the existing drugs [See, Stuart B. Levy, The Challenge of Antibiotic Resistance, in Scientific American, 46-53 (March, 1998)]. Therefore, new approaches to drug development are necessary to combat the ever-increasing number of antibiotic-resistant pathogens.

Classical penicillin-type antibiotics effect a single class of proteins known as autolysins. Thus, the development of new drugs which effect an alternative bacterial target protein would be desirable. Such a target protein ideally would be indispensable for bacterial survival. A enzyme such as bacterial RNAP would thus be a prime candidate for such drug development.

Therefore, there is a need to develop methods for identifying drugs that interfere with bacterial RNAP. Unfortunately, such identification has heretofore relied on serendipity and/or systematic screening of large numbers of natural and synthetic compounds. One superior method for drug screening relies on structure based rational drug design. In such cases, a three dimensional structure of the protein or peptide is determined and potential agonists and/or antagonists are designed with the aid of computer modeling [Bugg et al., Scientific American, December: 92-98 (1993); West et al., TIPS, 16:67-74 (1995); Dunbrack et al., Folding & Design, 2:27-42 (1997)].

Therefore, there is a need for obtaining a crystal of the bacterial RNAP bound to an inhibitor that is amenable to high resolution X-ray crystallographic analysis. In addition, there is a need for determining the three-dimensional structure of the RNAP bound to that inhibitor. Furthermore, there is a need for developing procedures of structure based rational drug design using such three-dimensional information. Finally, there is a need to employ such procedures to develop new anti-bacterial drugs.

The citation of any reference herein should not be construed as an admission that such reference is available as “Prior Art” to the instant application.

### SUMMARY OF THE INVENTION

The present invention provides crystals of RNA polymerase bound to an inhibitor. More particularly, the present invention provides crystals of the bacterial core RNA polymerase bound to rifampicin (the Rif-RNAP complex). In addition, the present invention also provides detailed three-dimensional structural data for the Rif-RNAP complex. The structural data obtained for the Rif-RNAP complex can be used for the rational design of drugs that inhibit bacterial cell proliferation. The present invention further provides methods of identifying and/or improving inhibitors of the bacterial core RNA polymerase which can be used in place of and/or in conjunction with other bacterial inhibitors including antibiotics.

One aspect of the present invention provides crystals of the bacterial core RNA polymerase bound to rifampicin that can effectively diffract X-rays for the determination of the atomic coordinates of the Rif-RNAP complex to a resolution of better than 5.0 Angstroms. In a preferred embodiment the crystal effectively diffracts X-rays for the determination of the atomic coordinates of the Rif-RNAP complex to a resolution of 3.5 Angstroms or better. In a particular embodiment the crystal of the Rif-RNAP complex effectively diffracts X-rays for the determination of the atomic coordinates to a resolution of 3.3 Angstroms or better.

In a particular embodiment the bacterial core RNA polymerase of the crystal is a thermophilic bacterial core RNA polymerase. In a preferred embodiment of this type the thermophilic bacterial core RNA polymerase is a Thermus aquaticus bacterial core RNA polymerase. Such a core RNA polymerase comprises a β′ subunit, a β subunit, and a pair of α subunits. Preferably, the core RNA polymerase further comprises an ω subunit. In a particular embodiment the β′ subunit has the amino acid sequence of SEQ ID NO: 1. In another embodiment the β subunit has the amino acid sequence of SEQ ID NO:2. In still another embodiment an α subunit has the amino acid sequence of SEQ ID NO:3. In still another embodiment an ω subunit has the amino acid sequence of SEQ ID NO:4.

In a preferred embodiment the core RNA polymerase is comprised of a β′ subunit having the amino acid sequence of SEQ ID NO:1, a β subunit having the amino acid sequence of SEQ ID NO:2, and a pair of α subunits having the amino acid sequence of SEQ ID NO:3. More preferably, this core RNA polymerase further comprises an ω subunit having the amino acid sequence of SEQ ID NO:4.

A crystal of the present invention may take a variety of forms all of which are included in the present invention. In a particular embodiment the crystal of the RNA polymerase has a space group of P4₁,2₁2 and a unit cell of dimensions of a=b=201 and c=294 Å.

The present invention further includes methods of preparing a crystal of the core RNA polymerase bound to an RNAP binding partner, e.g, an RNAP inhibitor such as rifampicin. A particular method comprises first growing a core bacterial RNA polymerase crystal in a buffered solution. One such buffered solution exemplified below, contains 40-45% saturated ammonium sulfate. In one such embodiment the growing is performed by batch crystallization. In another embodiment the growing is performed by vapor diffusion. In yet another embodiment the growing is performed by microdialysis.

The crystals can be subsequently soaked in a stabilization solution, (e.g., 2 M (NH₄)₂SO₄, 0.1 M Tris-HCl, pH 8.0, and 20 mM MgCl₂) with an RNAP binding partner such as rifampicin (0.1 mM rifampicin was added in the Example below). The RNAP/RNAP-binding partner are preferably incubated in the stabilization buffer for at least twelve hours. The crystals are then prepared for cryo-crystallography by soaking the RNAP/RNAP-binding partner complex in a stabilization buffer (e.g., 2 M (NH₄)₂SO₄, 0.1 M Tris-HCl, pH 8.0, and 20 mM MgCl₂ containing 50% (w/v) sucrose) before flash freezing. As exemplified below, crystals of the Rif-RNAP complex were prepared by soaking the Rif-RNAP complex for 30 minutes in stabilization buffer prior to flash freezing in liquid nitrogen.

Alternatively, the core RNA polymerase bound to an RNAP binding partner, e.g, an RNAP inhibitor such as rifampicin, can be co-crystallized under the conditions as described above.

Preferably the crystal of the Rif-RNAP complex effectively diffracts X-rays for the determination of the atomic coordinates of the Rif-RNAP complex to a resolution of better than 5.0 Angstroms. In a preferred embodiment the crystal effectively diffracts X-rays for the determination of the atomic coordinates of the Rif-RNAP complex to a resolution of 3.5 Angstroms or better. In a particular embodiment the crystal effectively diffracts X-rays for the determination of the atomic coordinates of the Rif-RNAP complex to a resolution of 3.3 Angstroms or better.

In a particular embodiment the crystal is grown by vapor diffusion. In one such embodiment the crystal is grown by hanging-drop vapor diffusion. In another embodiment the crystal is grown by sitting-drop vapor diffusion. Standard micro and/or macro seeding may be used to obtain a crystal of X-ray quality, i.e. a crystal that will diffract to allow resolution better than 5.0 Angstroms.

Still another aspect of the present invention comprises a method of using a crystal of the present invention and/or a dataset comprising the three-dimensional coordinates obtained from the crystal in a drug screening assay.

In addition, the present invention provides three-dimensional coordinates for the Rif-RNAP complex. In a particular embodiment the coordinates are for the Rif-RNAP complex using the Thermus aquaticus core RNA polymerase as disclosed in Table 2 (in Appendix following the Sequence Listing). Thus the dataset of Table 2 below, is part of the present invention. Furthermore, the dataset of Table 2 below, in a computer readable form is also part of the present invention. In addition, methods of using such coordinates (including in computer readable form) in the drug assays and drug screens as exemplified herein, are also part of the present invention. In a particular embodiment of this type, the coordinates contained in the dataset of Table 2 below, can be used to identify potential modulators of the core RNA polymerase. In a preferred embodiment, the modulator is designed to interfere with the bacterial RNAP, but not to interfere with the human RNAP.

Accordingly, the present invention provides methods of identifying an agent or drug that can be used to treat bacterial infections. One such embodiment comprises a method of identifying an agent for use as an inhibitor of bacterial RNA polymerase using a crystal of a Rif-RNAP complex and/or a dataset comprising the three-dimensional coordinates obtained from the crystal. In a particular embodiment the three-dimensional coordinates of the Rif-RNAP complex are determined using the Thermus aquaticus core RNA polymerase. Preferably the crystal of the Rif-RNAP complex effectively diffracts X-rays for the determination of the atomic coordinates to a resolution of, or better than 3.5 Angstroms. More preferably the crystal of the Rif-RNAP complex effectively diffracts X-rays for the determination of the atomic coordinates to a resolution of, or better than 3.3 Angstroms. Preferably the selection is performed in conjunction with computer modeling.

In one embodiment the potential agent is selected by performing rational drug design with the three-dimensional coordinates determined for the crystal. As noted above, preferably the selection is performed in conjunction with computer modeling. The potential agent is then contacted with the bacterial RNA polymerase and the activity of the bacterial RNA polymerase is determined (e.g., measured). A potential agent is identified as an agent that inhibits bacterial RNA polymerase when there is a decrease in the activity determined for the bacterial RNA polymerase.

In a preferred embodiment the method further comprises preparing a supplemental crystal containing the core RNA polymerase bound to the potential agent. Preferably the supplemental crystal effectively diffracts X-rays for the determination of the atomic coordinates to a resolution of better than 5.0 Angstroms, more preferably to a resolution equal to or better than 3.5 Angstroms, and even more preferably to a resolution equal to or better than 3.3 Angstroms. The three-dimensional coordinates of the supplemental crystal are then determined with molecular replacement analysis and a second generation agent is selected by performing rational drug design with the three-dimensional coordinates determined for the supplemental crystal. Preferably the selection is performed in conjunction with computer modeling.

As should be readily apparent the three-dimensional structure of a supplemental crystal can be determined by molecular replacement analysis or multiwavelength anomalous dispersion or multiple isomorphous replacement. A candidate drug is then selected by performing rational drug design with the three-dimensional structure determined for the supplemental crystal, preferably in conjunction with computer modeling. The candidate drug can then be tested in a large number of drug screening assays using standard biochemical methodology exemplified herein.

The method can further comprise contacting the second generation agent with a eukaryotic RNA polymerase and determining (e.g., measuring) the activity of the eukaryotic RNA polymerase. A potential agent is then identified as an agent for use as an inhibitor of bacterial RNA polymerase when there is significantly less change (a factor of two or more) in the activity of the eukaryotic RNA polymerase relative to that observed for the bacterial RNA polymerase. Preferably no, or alternatively minimal change (i.e., less than 15%) in the activity of the eukaryotic RNA polymerase is determined.

The present invention further provides a method of identifying an agent that inhibits bacterial growth using the crystal of a Rif-RNAP complex or a dataset comprising the three-dimensional coordinates obtained from the crystal. In a particular embodiment the three-dimensional coordinates of the Rif-RNAP complex are determined with the Thermus aquaticus core RNA polymerase.

Preferably the Rif-RNAP complex effectively diffracts X-rays for the determination of the atomic coordinates to a resolution of, or better than 3.5 Angstroms. More preferably the Rif-RNAP complex effectively diffracts X-rays for the determination of the atomic coordinates to a resolution of, or better than 3.3 Angstroms. Preferably the selection is performed in conjunction with computer modeling.

In one embodiment the potential agent is selected by performing rational drug design with the three-dimensional coordinates determined for the crystal of the Rif-RNAP complex. As noted above, preferably the selection is performed in conjunction with computer modeling. The potential agent is contacted with and/or added to a bacterial culture and the growth of the bacterial culture is determined. A potential agent is identified as an agent that inhibits bacterial growth when there is a decrease in the growth of the bacterial culture. The method can further comprise preparing a supplemental crystal containing the core RNA polymerase formed in the presence of the potential agent. Preferably the supplemental crystal effectively diffracts X-rays for the determination of the atomic coordinates to a resolution of better than 5.0 Angstroms, more preferably to a resolution equal to or better than 3.5 Angstroms, and even more preferably to a resolution equal to or better than 3.3 Angstroms. The three-dimensional coordinates of the supplemental crystal are then determined with molecular replacement analysis and a second generation agent is selected by performing rational drug design with the three-dimensional coordinates determined for the supplemental crystal. Preferably the selection is performed in conjunction with computer modeling. The candidate drug can then be tested in a large number of drug screening assays using standard biochemical methodology exemplified herein.

In a particular embodiment the second generation agent is contacted with a eukaryotic cell and the amount of proliferation of the eukaryotic cell is determined. A potential agent is identified as an agent for inhibiting bacterial growth when there is significantly less change (a factor of two or more) in the proliferation of the eukaryotic cell relative to that observed for the bacterial cell. Preferably no, or alternatively minimal change (i.e., less than 15%) in the proliferation of the eukaryotic cell is determined.

Computer analysis may be performed with one or more of the computer programs including: QUANTA, CHARMM, INSIGHT, SYBYL, MACROMODEL and ICM [Dunbrack et al., Folding & Design, 2:27-42 (1997)]. In a further embodiment of this aspect of the invention, an initial drug screening assay is performed using the three-dimensional structure so obtained, preferably along with a docking computer program. Such computer modeling can be performed with one or more Docking programs such as DOC, GRAM and AUTO DOCK [Dunbrack et al., Folding & Design, 2:27-42 (1997)].

It should be understood that in all of the drug screening assays provided herein, a number of iterative cycles of any or all of the steps may be performed to optimize the selection. For example, assays and drug screens that monitor the activity of the RNA polymerase in the presence and/or absence of a potential modulator (or potential drug) are also included in the present invention and can be employed as the sole assay or drug screen, or more preferably as a single step in a multi-step protocol for identifying modulators of bacterial proliferation and the like.

The present invention further provides the novel agents (modulators or drugs) that are identified by a method of the present invention, along with the method of using agents (modulators or drugs) identified by a method of the present invention, for inhibiting bacterial RNA polymerase and/or bacterial proliferation.

The present invention further provides an apparatus that comprises a representation of a Rif-RNAP complex. One such apparatus is a computer that comprises the representation of the Rif-RNAP complex in computer memory. In one embodiment, the computer comprises a machine-readable data storage medium which contains data storage material that is encoded with machine-readable data which comprises the atomic coordinates obtained from a crystal of the Rif-RNAP complex. Preferably the computer comprises a machine-readable data storage medium which contains data storage material that is encoded with machine-readable data which comprises the structural coordinates of Table 2. In one embodiment, the computer comprises a machine-readable data storage medium which contains data storage material that is encoded with machine-readable data which comprises the structural coordinates obtained from a crystal of the Rif-RNAP complex. More preferably the computer further comprises a working memory for storing instructions for processing the machine-readable data, a central processing unit coupled to both the working memory and to the machine-readable data storage medium for processing the machine readable data into a three-dimensional representation of the Rif-RNAP complex. In a preferred embodiment, the computer also comprises a display that is coupled to the central-processing unit for displaying the three-dimensional representation.

Accordingly, it is a principal object of the present invention to provide a crystal containing the Rif-RNAP complex.

It is a further object of the present invention to provide the three-dimensional coordinates of the Rif-RNAP complex for the Thermus aquaticus core RNA polymerase.

It is a further object of the present invention to provide methods for the rational design of drugs that inhibit prokaryotic RNA polymerase.

It is a further object of the present invention to provide methods of identifying drugs that can modulate bacterial proliferation.

It is a further object of the present invention to provide methods for the rational design of drugs that inhibit bacterial proliferation without negatively effecting human RNA polymerase.

It is a further object of the present invention to provide methods of identifying agents that can be used to treat bacterial infections in mammals, and preferably in humans.

These and other aspects of the present invention will be better appreciated by reference to the following drawings and Detailed Description.

### BRIEF DESCRIPTION OF THE DRAWINGS

FIG. 1 depicts the rifampicin (Rif) resistant regions of the RNAP β subunit. The bar on top schematically represents the E. coli β subunit primary sequence with amino acid numbering shown directly above. Gray boxes within the schematic indicate evolutionarily conserved regions among all prokaryotic, chloroplast, archaebacterial, and eukaryotic sequences labeled A-I at the top [Allison et al., Cell 42:599-610 (1985); Sweetser et al., Proc.Natl.Acad.Sci. USA 84:1192-1196 (1987)]. Red markings indicate the four clusters where Rif^{R} mutations have been identified in E. coli [Jin and Gross, J.Molec.Biol, 202:45-58 (1988); Lisitsyn et al., Bioorg Khim 10:127-128 (1984); Lisitsyn et al., Molec.Gen.Genet., 196:173-174 (1984); Ovchinnikov et al., Molec.Gen.Genet.190:344-348 (1983); Severinov et al., J.Biol.Chem., 268:14820-14825 (1993); Severinov et al., Molec.Gen.Genet., 244:120-126 (1994)] denoted as the N-terminal cluster (N), and clusters I, II and III (I, II, III). Directly below is a sequence alignment spanning these regions of the E. coli (E.c.), T. aquaticus (T.a.), and M. tuberculosis (M. t) RNAP β subunits. Amino acids that are identical to E. coli are shaded dark gray, and those that are homologous (ST, RK, DE, NQ, FYWIV) are shaded light gray. Mutations that confer Rif^{R} in E. coli and M. tuberculosis are indicated directly above (for E. coli) or below (for M. tuberculosis) as follows: Δ for deletions, Ω for insertions, and colored dots for amino acid substitutions (substitutions at each position are indicated in single-amino acid code in columns above or below the positions).

Color-coding for the amino acid substitutions (for reference to subsequent figures):

(i) yellow, residues that interact directly with the bound rifampicin (see FIGS. 4a-4b);

(ii) green, residues that are too far away from the rifampicin for direct interaction (see FIG. 5a-5b); and

(iii) purple, three positions that are substituted with high frequency (noted as a % immediately below the substitutions) in clinical isolates of Rif^{R} M. tuberculosis [Ramaswamy and Musser, Tubercle and Lung Disease 79:3-29 (1998)].

Below the three prokaryotic sequences is a sequence alignment of three eukaryotic sequences with shading as above. The dots indicate a gap in the alignment.

FIGS. 2a-2d show that the rifampicin inhibition of Taq RNAP. FIG. 2a depicts autoradiographs showing the radioactive RNA produced by Taq (lanes 1-7) and E. coli (lanes 8-13) RNAP holoenzymes transcribing a template containing the T7 A1 promoter and the tR2 terminator, analyzed on a 15% polyacrylamide gel and quantitated by phosphorimagery. In the absence of rifampicin (lanes 1 and 8), the major RNA products from each RNAP correspond to a trimeric abortive product (CpApU), a 105 nucleotide terminated transcript (Term), and a 127 nucleotide runoff transcript (Run off). Lanes 2-7 and 9-13 show the effects of increasing concentrations of rifampicin. FIG. 2b shows the quantitated results, where the amounts of each product (normalized to 100% for the Run off and Term transcripts in the absence of rifampicin, and for CpApU at the highest concentration of rifampicin) are plotted as a function of rifampicin concentration. FIG. 2c shows the distance between the bound rifampicin and the initiating substrate (i-site) of E. coli and Taq RNAP holoenzymes measured using chimeric Rif-nucleotide compounds as previously described [Mustaev et al., Proc.Nat.Acad.Sci. USA 91:12036-12040 (1994)]. Rif-nucleotide compounds (Rif-(CH2)n-Ap) with different linker lengths, n (indicated above each lane) were bound to RNAP, then extended in a specific transcription reaction with α-[³²P]UTP by the RNAP catalytic activity. The products were analyzed on a 23% polyacrylamide gel, visualized by autoradiography, and quantitated by phosphorimagery. FIG. 2d shows the quantitated results where the product yield (as % activity normalized to 100% at the highest level) is plotted as a function of the Rif-nucleotide linker length (n).

FIGS. 3a-3c show the Rif-RNAP co-crystal structure. FIG. 3a is a stereoview of the Rif-binding pocket of Taq core RNAP, generated using O [Jones et al., Acta Cryst, A 47:110-119 (1991)]. Carbon atoms of the RNAP β subunit are cyan or yellow (residues within 4 Å of the rifampicin), while carbon atoms of the inhibitor are orange. Oxygen atoms are red, nitrogen atoms are blue, and sulfur atoms are green. Electron density, calculated using (|Fₒ^{Rif}−Fₒ^{nat}|) coefficients is shown (orange) for the Rif only (contoured at 3.5σ), and was computed using phases from the final refined RNAP model with the rifampicin omitted [see U.S. Ser. No. 09/396,651, Filed Sep. 15, 1999, the contents of which are hereby incorporated by reference in their entireties.] Here, “Rif” denotes the Rif-RNAP co-crystal, and “native” denotes the native core RNAP crystal. FIG. 3b shows the three-dimensional structure of Taq core RNAP in complex with rifampicin generated using GRASP [Nicholls et al., Proteins Structure, Function and Genetics 11:281-296 (1991)]. The backbone of the RNAP structure is shown as tubes, along with the color-coded transparent molecular surface (β, cyan; β′, pink; ω, white; the α-subunits are behind the RNAP and are not visible). The Mg²⁺ ion chelated at the active site is shown as a magenta sphere. The rifampicin is shown as CPK atoms (carbon, orange; oxygen, red; nitrogen, blue). FIG. 3c is the structural formula of rifampicin. Features of the structure discussed in the text are color-coded (ansa bridge, blue; napthol ring, green). The four oxygen atoms critical for rifampicin activity [Arora, Acta Crystall. B37: 152-157 (1981); Arora, Molecular Pharmacology 23:133-140 (1983); Arora, J.Med.Chem. 28:1099-1102 (1985); Arora and Main, J. Antibiot. 37:178-181 (1984); Brufani et al., J. Molec.Biol. 87:409-435 (1974); Lancini and Zanichelli, In Structure-activity Relationship in Semisynthetic Antibiotics, D. Perlaman, ed. (Academic Press), pp. 531-600 (1977); Sensi et al., Rev.Infect.Dis., 5 Supp.3:402-406 (1983)] are shaded with red circles.

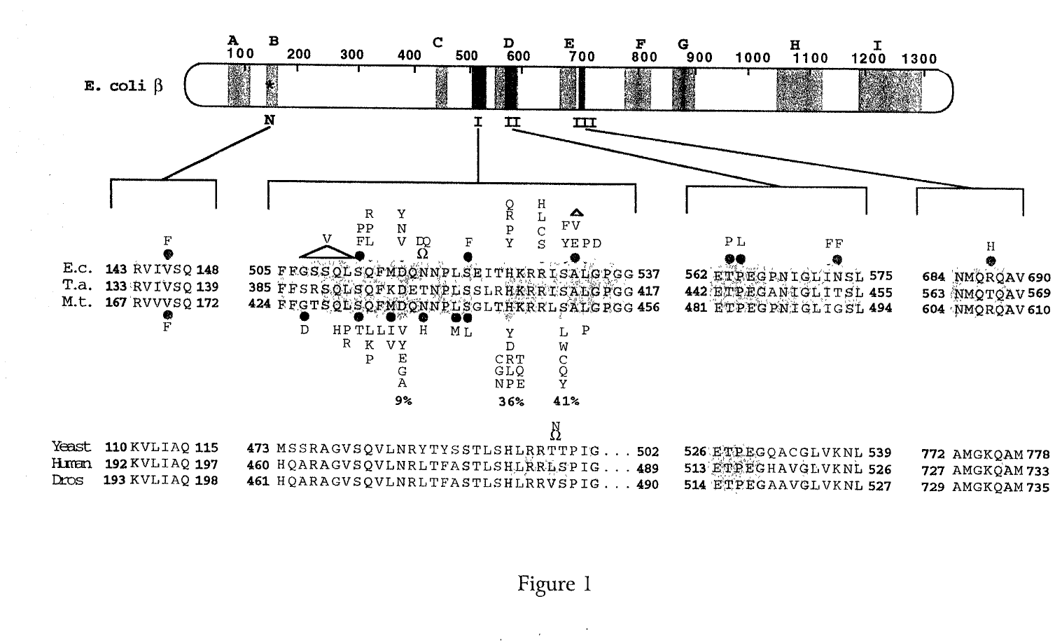

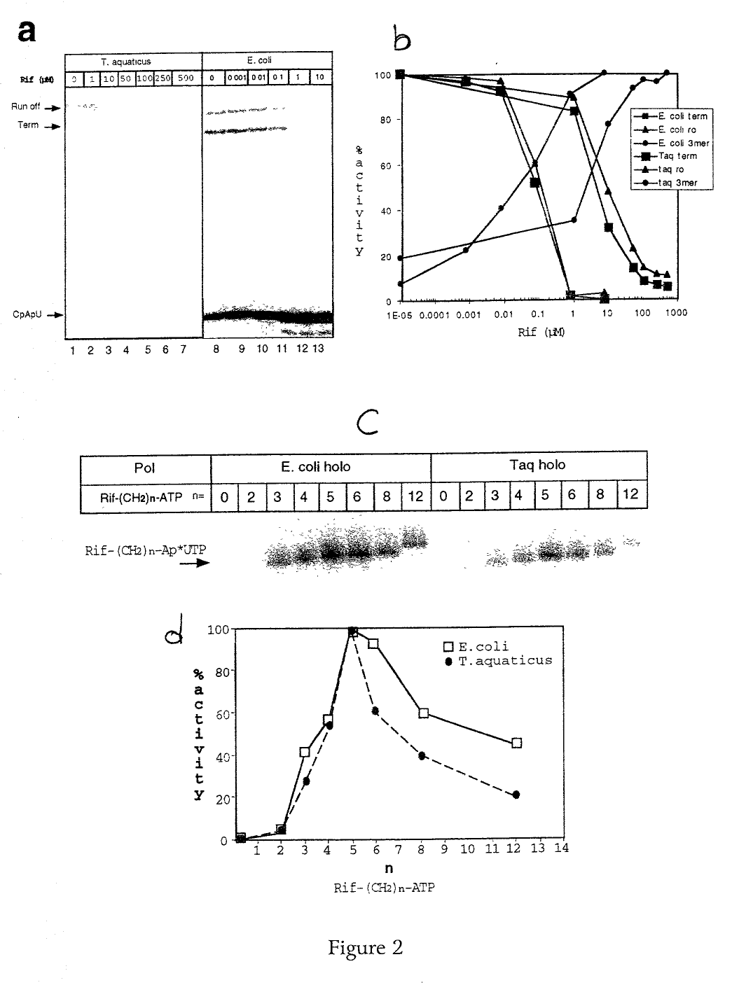

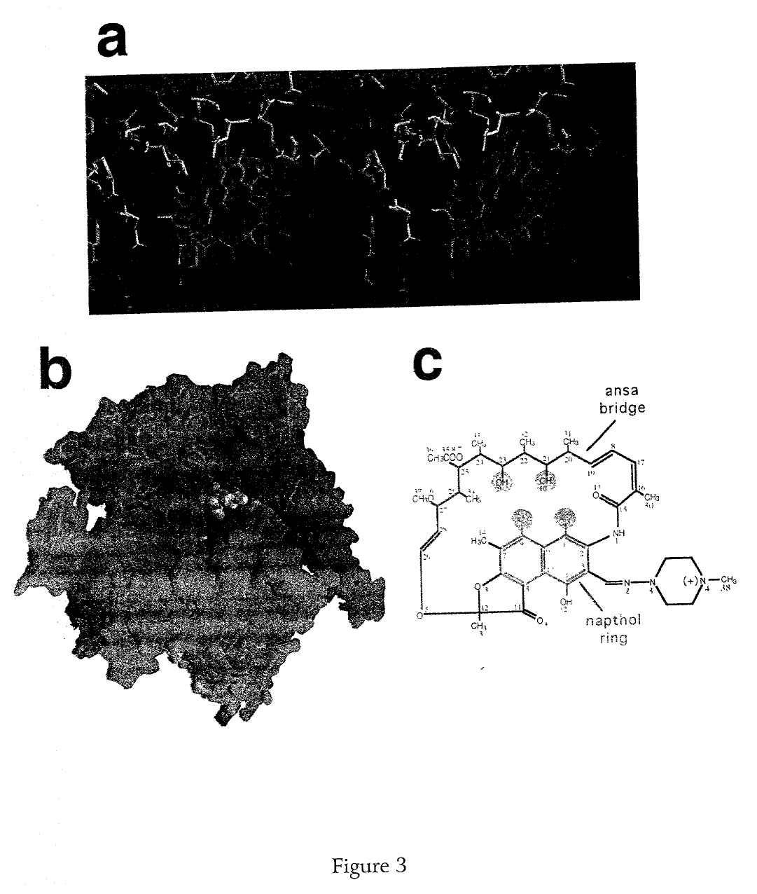

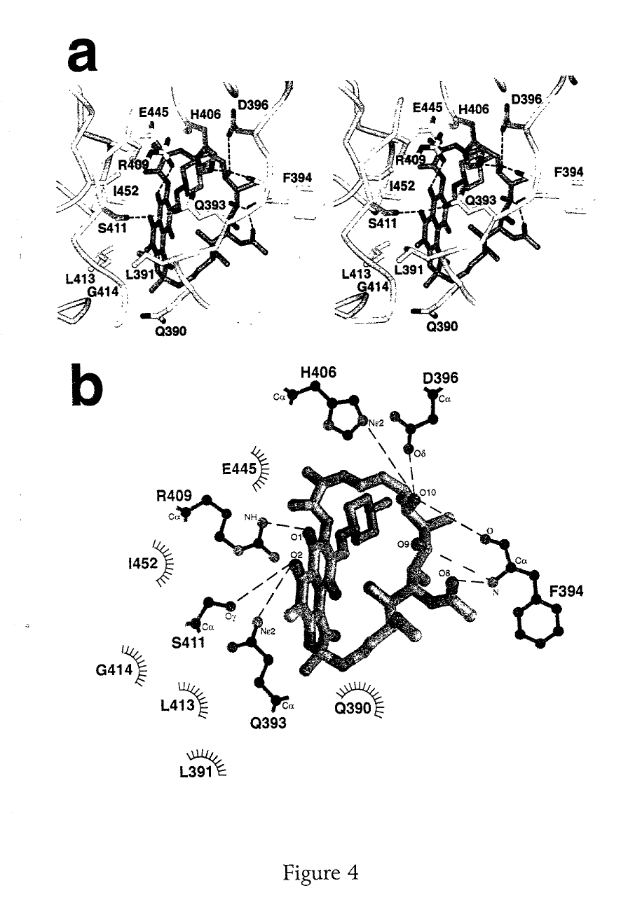

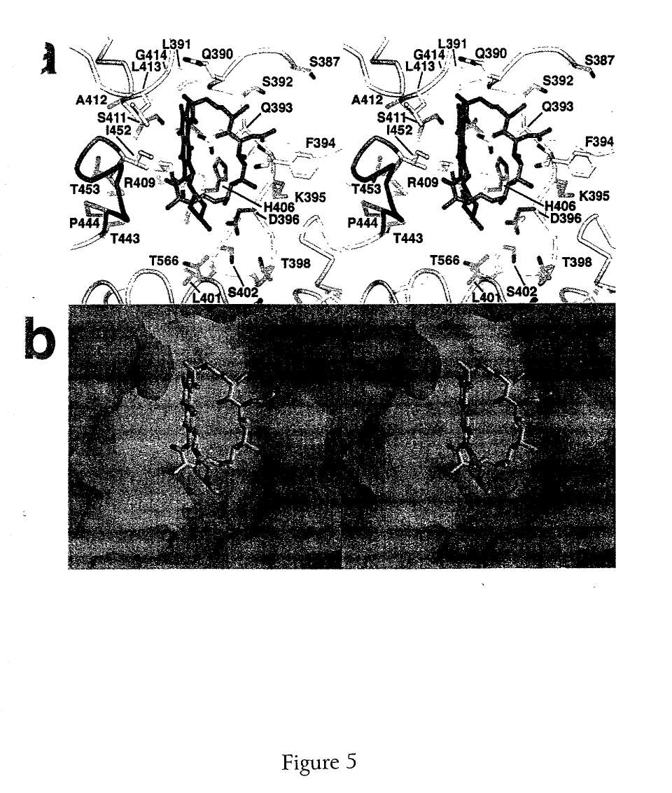

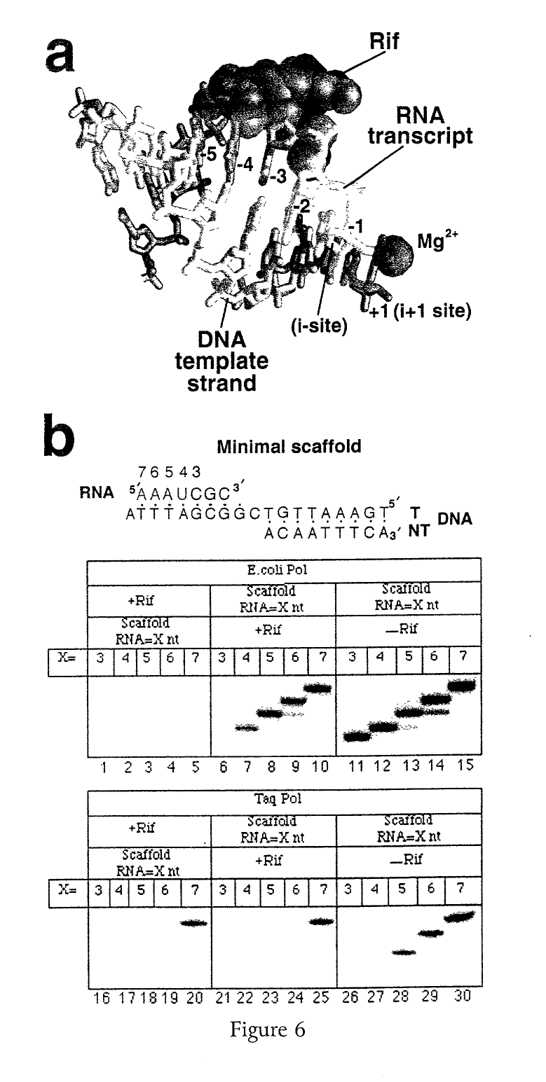

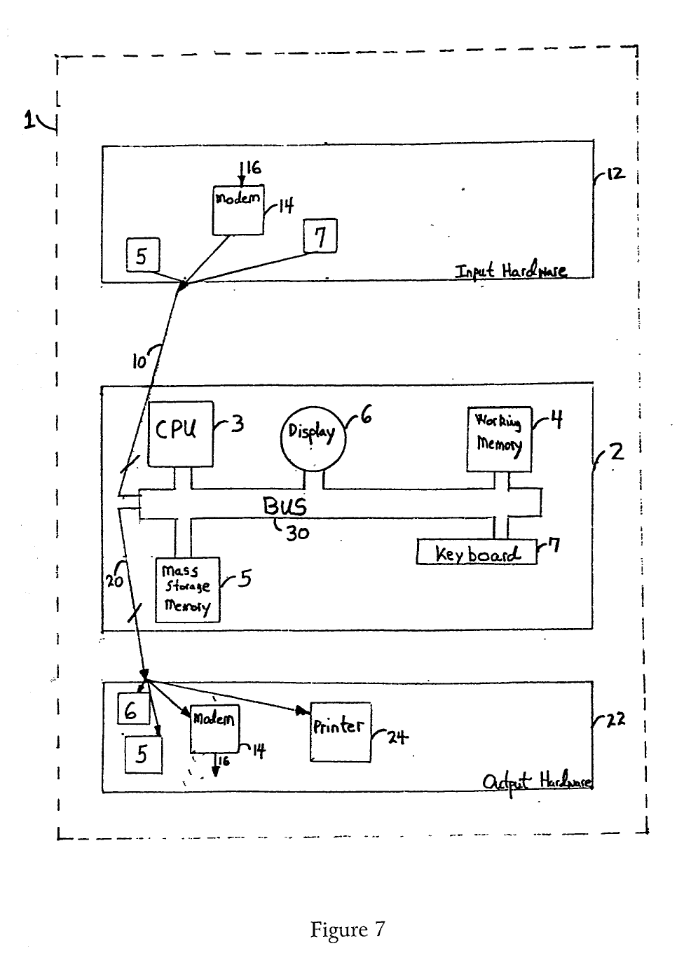

FIGS. 4a-4b depict the detailed interactions of rifampicin with RNAP. FIG. 4a is a stereoview of the Taq RNAP Rif binding pocket complexed with rifampicin, generated using RIBBONS [Carson, J.Appl.Crystall., 24:958-961 (1991)], showing residues that interact directly with the inhibitor. The backbone of the β subunit is shown as a cyan ribbon. Side chains (and backbone atoms of F394) of residues within 4 Å of rifampicin are shown. Carbon atoms are orange (Rif), magenta (three residues substituted in M. tuberculosis Rif^{R} clinical isolates with high frequency, see FIG. 1), or yellow; oxygen atoms are red; nitrogen atoms are blue. The view is from above the β subunit, looking through β to the rifampicin, but with obscuring parts of β removed. Potential hydrogen bonds between protein atoms and rifampicin are shown as dashed lines. FIG. 4b shows a schematic drawing of RNAP β subunit interactions with rifampicin, modified from LIGPLOT [Wallace et al., Protein Engineering 8:127-134 (1995)]. Residues forming van-der-Waals interactions are indicated: those participating in hydrogen bonds are shown in a ball-and-stick representation, with hydrogen bonds depicted as dashed lines, carbon atoms of the protein are black, while carbon atoms of rifampicin are orange. Oxygen atoms are red and nitrogen atoms are blue.

FIGS. 5a-5b show the rifampicin binding pocket and Rif^{R} mutants as stereoviews of the Taq RNAP Rif binding pocket complexed with rifampicin. The view is the same in FIGS. 5a and 5b and is rotated approximately 180° about the horizontal axis from the view of FIG. 4a. This view is from the middle of the main RNAP channel, looking towards the rifampicin, with the β subunit behind. FIG. 5a shows the backbone of the β subunit as a cyan ribbon, but with a highly conserved segment of region D (443-451, see text) colored red. Side chains (and backbone atoms of F394) of residues where substitutions confer Rif^{R} (see FIG. 1) are shown. Carbon atoms are orange (Rif), magenta (three residues substituted in M. tuberculosis Rif^{R} clinical isolates with high frequency, see FIG. 1), yellow (other residues that interact directly with rifampicin, as in FIG. 4), or green (all other Rif^{R} positions). Oxygen atoms are colored red; nitrogen atoms are blue. The depiction was generated using RIBBONS [Carson, J.Appl.Crystall., 24:958-961 (1991)]. The β subunit is shown in FIG. 5b as a cyan molecular surface, with a highly conserved segment of region D colored red, and surface exposed Rif^{R} positions colored yellow (within 4 Å of the Rif) or green. The depiction was generated using GRASP [Nicholls et al., Proteins Structure, Function and Genetics 11:281-296 (1991)]. FIGS. 6a and 6b show the mechanism of RNAP inhibition by rifampicin. The RNAP active site Mg²⁺ (magenta sphere) and the 9-basepair RNA/DNA hybrid (from +1 to −8) from a model of the ternary elongation complex [Korzheva et al., Science 289:619-625 (2000)] are shown in FIG. 6a. The RNAP itself and the rest of the nucleic acids are omitted for clarity. The incoming nucleotide substrate at the +1 position is colored green, the −1 and −2 positions, which can be accommodated in the presence of rifampicin, are colored yellow. The RNA further upstream (−3 to −8), which cannot be accommodated in the presence of rifampicin is colored pink. The template strand of the DNA is colored grey. Also shown is a CPK representation of rifampicin as it would be positioned in its binding site on the β subunit (carbon atoms, orange; oxygen, red; nitrogen, blue). The rifampicin is partially transparent, illustrating the RNA nucleotides at −3 to −5 that sterically clash. This depiction was generated using GRASP [Nicholls et al., Proteins Structure, Function and Genetics 11:281-296 (1991)]. The structure of the minimal scaffold systems with RNA lengths from 3-7 nucleotides (labeled above the RNA chain) are shown in FIG. 6b [Korzheva et al., Science 289:619-625 (2000)]. The results are presented below as autoradiographs of the radioactive RNAs produced by E. coli (lanes 1-15) or Taq (lanes 16-30) core RNAPs transcribing the minimal scaffolds with the indicated lengths of RNA (‘X=’) and analyzed on a 23% polyacrylamide gel. Lanes 1-10 and 16-25 demonstrate the effect of rifampicin inhibition on transcription when it was bound by RNAP either before (lanes 1-5 and 16-20) or after (lanes 6-10 and lanes 21-25) addition of the scaffold. Lanes 11-15 and 26-30 show elongation of the same scaffolds in the absence of rifampicin. The RNA with the critical length of 3 nucleotides which cannot be elongated by E.coli RNAP in the presence of rifampicin regardless of the order of rifampicin and scaffold addition (lanes 1,6) is colored red. The RNAs of 4-7 nucleotides (colored green) were extended by E. coli RNAP when added before rifampicin (lanes 6-10).

FIG. 7 depicts a schematic of a computer comprising a central processing unit (“CPU”), a working memory, a mass storage memory, a display terminal, and a keyboard that are interconnected by a conventional bidirectional system bus. The computer can be used to display and manipulate the structural data of the present invention.

### DETAILED DESCRIPTION OF THE INVENTION

The present invention provides crystals of a bacterial core RNA polymerase bound to an inhibitor. The present invention further provides the structural coordinates for a bacterial core RNA polymerase bound to rifampicin (Rif-RNAP complex) and methods of using such structural coordinates in drug assays. More particularly, the present invention provides the structural coordinates for the Rif-RNAP complex with the Thermus aquaticus core RNA polymerase (see Table 2 in Appendix following the Sequence Listing).

Rifampicin (Rif) is one of the most potent and broad-spectrum antibiotics against bacterial pathogens and is a key component of anti-tuberculosis therapy, stemming from its inhibition of the bacterial RNA polymerase (RNAP). The X-ray crystal structure of Thermus aquaticus core RNA polymerase reveals a ‘crab-claw’ shaped molecule with a 27 Å wide internal channel [see U.S. Ser. No. 09/396,651, Filed Sep. 15, 1999, the contents of which are hereby incorporated by reference in their entireties]. As disclosed herein, rifampicin binds in a pocket of the RNAP β subunit deep within the DNA/RNA channel, but more than 12 Å away from the active site the crystal structure of Thermus aquaticus core RNAP complexed with rifampicin. The structure, combined with biochemical results disclosed herein, explains the effects of rifampicin on RNAP function and indicates that the inhibitor acts by directly blocking the path of the elongating RNA when the transcript becomes 2 to 3 nucleotides in length.

The three-dimensional structure disclosed herein demonstrates that rifampicin binds the Taq core RNAP with a close complementary fit in a pocket between two structural domains of the RNAP β subunit. Only small, local conformational changes of both the inhibitor and the protein were observed. The binding site is deep within the main RNAP channel, but the closest approach of the inhibitor to the RNAP active site Mg²⁺ is more than 12 Å (FIG. 3b, below). The Rif binding pocket is surrounded by the 23 known positions where amino acid substitutions confer Rif^{R} (FIG. 5, below). Twelve of these residues are close enough to interact directly with the rifampicin (FIGS. 4a-4b, below). Predominant are van-der-Waals interactions with hydrophobic side-chains near the napthol ring of rifampicin, and potential hydrogen bond interactions with 5 polar groups of rifampicin (2 on the napthol ring, and 3 on the ansa bridge), 4 of which have been shown to be essential for rifampicin activity. The remaining known Rif^{R} mutants are one layer removed from the rifampicin itself, and are likely to affect rifampicin binding through small structural distortions of the Rif binding pocket.

Therefore the structure disclosed herein explains the effects of rifampicin on RNAP function determined from detailed biochemical and kinetic studies. In combination with a model of the ternary transcription complex, the structure indicates that the predominant effect of rifampicin is to directly block the path of the elongating RNA transcript at the 5′-end when the transcript becomes either 2 or 3 nucleotides in length, depending on the 5′-phosphorylation state of the 5′-nucleotide (FIGS. 6a-6b, below). In this view, rifampicin binds the Rif binding site of the RNAP holoenzyme either before or after the binding of the DNA template and formation of the open complex. Indeed, the binding of the DNA template and the formation of the open complex are not affected by the presence of rifampicin. However, rifampicin has its effect after the nucleotide substrates binds their sites in the RNAP active site. Thus the initiating nucleotide substrate binds the RNAP i-site with a small, approximately 2-fold increase in the apparent Km due to the presence of rifampicin, while the second nucleotide binds in the i+1 site with little notice of the rifampicin. More or less normally, the RNAP then catalyzes the formation of a phosphodiester bond between the two nucleotides. If the initiating nucleoside bears a 5′-triphosphate, the subsequent translocation of the RNAP attempts to move the 2-nucleotide RNA transcript upstream such that the i+1 nucleotide occupies the i-site (−1 position), and the i-site nucleotide moves into the −2 position (FIG. 6a, below). The movement of the 5′-nucleotide into the −2 position, however, results in a severe steric clash with the rifampicin. The molecular details of the ensuing events are unclear, but in the end the RNAP remains at the same template position, the 2-nucleotide transcript is released, and the futile cycle begins again. If the 5′-nucleoside contains a di- or a mono-phosphate at its 5′-end (or if it's unphosphorylated), then after the synthesis of the first phosphodiester bond, the RNAP can translocate normally and the steric clash of the transcript with the bound rifampicin occurs during the translocation of the 3-nucleotide transcript following the synthesis of the second phosphodiester bond.

The present invention exploits the structural information described herein, including the structural coordinates disclosed in Table 2, and provides methods of identifying agents or drugs that can be used to control the proliferation of bacteria, e.g., for use as treatments for bacterial infections.

Therefore, if appearing herein, the following terms shall have the definitions set out below:

As used herein the term “core RNA polymerase” minimally comprises the subunit composition of α₂ββ′ which is evolutionarily conserved from bacteria to man. Preferably the core RNA polymerase further comprises the co subunit. The three-dimensional structure of the Thermus aquaticus core RNA polymerase was disclosed in U.S. Ser. No. 09/396,651, Filed Sep. 15, 1999, the contents of which are hereby incorporated by reference in their entireties.

As used herein “Rif-RNAP” is used interchangeably with the “Rif-RNAP complex” and comprises the binding complex of rifampicin with the core RNA polymerase as disclosed in the Example below. The structural coordinates for a crystal of Rif-RNAP are listed in Table 2 (in Appendix following the Sequence Listing).

As used herein an “RNAP binding partner” is a small organic molecule that binds to RNAP. Preferably the RNAP binding partner is an inhibitor of the catalytic and/or the transcriptional activity of RNAP. Rifampicin is a particular binding partner of RNAP that is exemplified below.

As used herein, the “transcriptional activity of RNAP” includes the ability of RNAP to carry out the elongation of the RNA transcript during transcription. Thus, whereas the catalytic activity of RNAP includes the binding of the enzyme to the nucleotide substrates and the subsequent formation of the phosphodiester bond between the two substrates, the transcriptional activity includes the RNAP dependent elongation of the RNA transcript at the 5′-end.

As used herein an “active RNA polymerase” is an RNA polymerase that minimally contains a pair of α subunits, a β′ subunit, and a β subunit; or fragments thereof, but still retains at least 25% of the catalytic and/or transcriptional activity of the core RNA polymerase made up of the full length α, β′ , and β subunits. Thus active RNA polymerases can comprise fragments of the α subunit and/or β′ subunit and/or β subunit.

As used herein a “small organic molecule” is an organic compound [or organic compound complexed with an inorganic compound (e.g., metal)] that has a molecular weight of less than 3 Kd.

As used herein the term “about” means within 10 to 15%, preferably within 5 to 10%. For example an amino acid sequence that contains about 60 amino acid residues can contain between 51 to 69 amino acid residues, more preferably 57 to 63 amino acid residues.

As used herein a polypeptide or peptide “consisting essentially of” or that “consists essentially of” a specified amino acid sequence is a polypeptide or peptide that retains the general characteristics, e.g., activity of the polypeptide or peptide having the specified amino acid sequence and is otherwise identical to that protein in amino acid sequence except it consists of plus or minus 10% or fewer, preferably plus or minus 5% or fewer, and more preferably plus or minus 2.5% or fewer amino acid residues.

As used herein, and unless otherwise specified, the terms “agent”, “potential drug”, “test compound” or “potential compound” are used interchangeably, and refer to chemicals which potentially have a use as a modulator (and preferably as an inhibitor) of bacterial RNA polymerase. More preferably, an agent is a drug that can be used to treat and/or prevent bacterial infection. Therefore, such “agents”, “potential drugs”, and “potential compounds” may be used, as described herein, in drug assays and drug screens and the like.

### Nucleic Acids Encoding Subunits of Bacterial RNA Polymerases

The present invention contemplates isolation of nucleic acids encoding a subunit of an RNA polymerase including a full length, i.e., naturally occurring form of the RNA polymerase from any prokaryotic source, preferably a thermophilic bacterial source. The present invention further provides for subsequent modification of the nucleic acid to generate a fragment or modification of the subunit that can still be used to form a core RNA polymerase that will crystallize.

In accordance with the present invention there may be employed conventional molecular biology, microbiology, and recombinant DNA techniques within the skill of the art. Such techniques are explained fully in the literature [see, e.g., Sambrook and Russell Molecular Cloning: A Laboratory Manual, Third Edition (2001) Vols. I-III, Cold Spring Harbor Laboratory Press, Cold Spring Harbor, N.Y. (herein “Sambrook and Russell, 2001”)].

Therefore, if appearing herein, the following terms shall have the definitions set out below.

As used herein, the term “gene” refers to an assembly of nucleotides that encode a polypeptide, and includes cDNA and genomic DNA nucleic acids.

A “vector” is a replicon, such as plasmid, phage or cosmid, to which another DNA segment may be attached so as to bring about the replication of the attached segment.

A “replicon” is any genetic element (e.g., plasmid, chromosome, virus) that functions as an autonomous unit of DNA replication in vivo, i.e., capable of replication under its own control.

A “cassette” refers to a segment of DNA that can be inserted into a vector at specific restriction sites. The segment of DNA encodes a polypeptide of interest, and the cassette and restriction sites are designed to ensure insertion of the cassette in the proper reading frame for transcription and translation.

A cell has been “transfected” by exogenous or heterologous DNA when such DNA has been introduced inside the cell. A cell has been “transformed” by exogenous or heterologous DNA when the transfected DNA effects a phenotypic change. Preferably, the transforming DNA should be integrated (covalently linked) into chromosomal DNA making up the genome of the cell.

“Heterologous DNA” refers to DNA not naturally located in the cell, or in a chromosomal site of the cell. Preferably, the heterologous DNA includes a gene foreign to the cell.

A “heterologous nucleotide sequence” as used herein is a nucleotide sequence that is added to a nucleotide sequence of the present invention by recombinant methods to form a nucleic acid which is not naturally formed in nature. Such nucleic acids can encode chimeric and/or fusion proteins. Thus the heterologous nucleotide sequence can encode peptides and/or proteins which contain regulatory and/or structural properties. In another such embodiment the heterologous nucleotide can encode a protein or peptide that functions as a means of detecting the protein or peptide encoded by the nucleotide sequence of the present invention after the recombinant nucleic acid is expressed. In still another embodiment the heterologous nucleotide can function as a means of detecting a nucleotide sequence of the present invention. A heterologous nucleotide sequence can comprise non-coding sequences including restriction sites, regulatory sites, promoters and the like.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength [see Sambrook and Russell, 2001, supra]. The conditions of temperature and ionic strength determine the “stringency” of the hybridization. For preliminary screening for homologous nucleic acids, low stringency hybridization conditions, corresponding to a Tₘ of 55°, can be used, e.g., 5×SSC, 0.1% SDS, 0.25% milk, and no formamide; or 30% formamide, 5×SSC, 0.5% SDS). Moderate stringency hybridization conditions correspond to a higher Tₘ, e.g., 40% formamide, with 5× or 6×SSC. High stringency hybridization conditions correspond to the highest Tₘ, e.g., 50% formamide, 5× or 6×SSC. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived [see Sambrook and Russell, 2001, supra]. For hybridization with shorter nucleic acids, i.e., oligonucleotides, the position of mismatches becomes more important, and the length of the oligonucleotide determines its specificity [see Sambrook and Russell, 2001, supra]. Preferably a minimum length for a hybridizable nucleic acid is at least about 12 nucleotides; preferably at least about 18 nucleotides; and more preferably the length is at least about 27 nucleotides; and most preferably 36 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C. In a particular embodiment the hybridization and wash conditions are identical,

“Homologous recombination” refers to the insertion of a foreign DNA sequence of a vector in a chromosome. Preferably, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

A DNA “coding sequence” is a double-stranded DNA sequence which is transcribed and translated into a polypeptide in a cell in vitro or in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. If the coding sequence is intended for expression in a eukaryotic cell, a polyadenylation signal and transcription termination sequence will usually be located 3′ to the coding sequence.

Transcriptional and translational control sequences are DNA regulatory sequences, such as promoters, enhancers, terminators, and the like, that provide for the expression of a coding sequence in a host cell. In eukaryotic cells, polyadenylation signals are control sequences.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

A coding sequence is “under the control” of transcriptional and translational control sequences in a cell when RNA polymerase transcribes the coding sequence into mRNA, which may then be trans-RNA spliced and translated into the protein encoded by the coding sequence.

As used herein, the term “sequence homology” in all its grammatical forms refers to the relationship between proteins that possess a “common evolutionary origin,” including proteins from superfamilies (e.g., the immunoglobulin superfamily) and homologous proteins from different species (e.g., myosin light chain, etc.) [Reeck et al., Cell, 50:667 (1987)].

Accordingly, the term “sequence similarity” in all its grammatical forms refers to the degree of identity or correspondence between nucleic acid or amino acid sequences of proteins that do not share a common evolutionary origin [see Reeck et al., 1987, supra]. However, in common usage and in the instant application, the term “homologous,” when modified with an adverb such as “highly,” may refer to sequence similarity and not a common evolutionary origin.

In a specific embodiment, two DNA sequences are “substantially homologous” or “substantially similar” when at least about 50% (preferably at least about 75%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Sambrook and Russell, 2001, supra.

Similarly, in a particular embodiment, two amino acid sequences are “substantially homologous” or “substantially similar” when greater than 30% of the amino acids are identical, or greater than about 60% are similar (functionally identical). Preferably, the similar or homologous sequences are identified by alignment using, for example, the GCG (Genetics Computer Group, Program Manual for the GCG Package, Version 7, Madison, Wis.) pileup program with the default parameters.

The term “corresponding to” is used herein to refer similar or homologous sequences, whether the exact position is identical or different from the molecule to which the similarity or homology is measured. Thus, the term “corresponding to” refers to the sequence similarity, and not the numbering of the amino acid residues or nucleotide bases.

A gene encoding an RNA polymerase, including genomic DNA or cDNA, can be isolated from any source, particularly from a thermophilic bacterial source. In view and in conjunction with the present teachings, methods well known in the art, as described above can be used for obtaining the genes encoding an RNA polymerase from any source [see, e.g., Sambrook and Russell, 2001, supra].

Accordingly, any cell potentially can serve as the nucleic acid source for the molecular cloning of a gene encoding RNA polymerase. The DNA may be obtained by standard procedures known in the art from cloned DNA (e.g., a DNA “library”), and preferably is obtained from a cDNA library, by cDNA cloning, or by the cloning of genomic DNA, or fragments thereof, purified from the desired cell [See, for example, Sambrook and Russell, 2001,supra]. Clones derived from genomic DNA may contain regulatory and intron DNA regions in addition to coding regions; clones derived from cDNA will not contain intron sequences. Whatever the source, the gene should be molecularly cloned into a suitable vector for propagation of the gene.

The present invention also relates to cloning vectors containing genes encoding analogs and derivatives of RNA polymerase including and fragments of the various subunits, that can form active forms of RNA polymerase. Included are homologs of RNA polymerase and fragments thereof, from other species. Therefore the production and use of derivatives and analogs related to RNA polymerase are within the scope of the present invention.

RNA polymerase derivatives can be made by altering encoding nucleic acid sequences by substitutions, additions or deletions including to provide for functionally equivalent molecules. Preferably, derivatives are made that are capable of forming crystals with ligands (e.g., inhibitors) of the RNA polymerase with the crystals capable of effectively diffracting X-rays for the determination of the atomic coordinates of the protein-ligand complex to a resolution of better than 5.0 Angstroms, preferably to a resolution equal to or better than 3.5 Angstroms.

Due to the degeneracy of nucleotide coding sequences, other DNA sequences which encode substantially the same amino acid sequence as a RNA polymerase gene may be used in the practice of the present invention. These include but are not limited to allelic genes, homologous genes from other species, and nucleotide sequences comprising all or portions of RNA polymerase genes which are altered by the substitution of different codons that encode the same amino acid residue within the sequence, thus producing a silent change. Likewise, the RNA polymerase derivatives of the invention include, but are not limited to, those containing, as a primary amino acid sequence, all or part of the amino acid sequence of a RNA polymerase including altered sequences in which functionally equivalent amino acid residues are substituted for residues within the sequence resulting in a conservative amino acid substitution. For example, one or more amino acid residues within the sequence can be substituted by another amino acid of a similar polarity, which acts as a functional equivalent, resulting in a silent alteration. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations will not be expected to affect apparent molecular weight as determined by polyacrylamide gel electrophoresis, or isoelectric point.

Particularly preferred substitutions are:

Lys for Arg and vice versa such that a positive charge may be maintained;

Glu for Asp and vice versa such that a negative charge may be maintained;

Ser for Thr such that a free -OH can be maintained; and

Gln for Asn such that a free NH₂ can be maintained.

Amino acid substitutions may also be introduced to substitute an amino acid with a particularly preferable property. For example, a Cys may be introduced at a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces β-turns in the protein's structure.

The genes encoding RNA polymerase derivatives and analogs of the invention can be produced by various methods known in the art. The manipulations which result in their production can occur at the gene or protein level. For example, the cloned RNA polymerase gene sequence can be modified by any of numerous strategies known in the art [Sambrook and Russell, 2001, supra]. The sequence can be cleaved at appropriate sites with restriction endonuclease(s), followed by further enzymatic modification if desired, isolated, and ligated in vitro. In the production of the gene encoding a derivative or analog of RNA polymerase, care should be taken to ensure that the modified gene remains within the same translational reading frame as the RNA polymerase gene, uninterrupted by translational stop signals, in the gene region where the desired activity is encoded.

Additionally, the RNA polymerase-encoding nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Preferably, such mutations enhance the functional activity and crystallization properties of the mutated RNA polymerase gene product. Any technique for mutagenesis known in the art can be used, including but not limited to, in vitro site-directed mutagenesis [Hutchinson, et al., J.Biol.Chem. 253:6551 (1978); Zoller and Smith, DNA 3:479-488 (1984); Oliphant et al., Gene 44:177 (1986); Hutchinson et al., Proc.Natl.Acad.Sci.U.S.A. 83:710 (1986)], use of TAB® linkers (Pharmacia), etc. PCR techniques are preferred for site directed mutagenesis [see Higuchi, “Using PCR to Engineer DNA”, in PCR Technology: Principles and Applications for DNA Amplification, H. Erlich, ed., Stockton Press, Chapter 6, pp. 61-70 (1989)].

The identified and isolated gene can then be inserted into an appropriate cloning vector. A large number of vector-host systems known in the art may be used. Possible vectors include, but are not limited to, plasmids or modified viruses, but the vector system must be compatible with the host cell used. Examples of vectors include, but are not limited to, E. coli, bacteriophages such as lambda derivatives, or plasmids such as pBR322 derivatives or pUC plasmid derivatives, e.g., pGEX vectors, pmal-c, pFLAG, etc. The insertion into a cloning vector can, for example, be accomplished by ligating the DNA fragment into a cloning vector which has complementary cohesive termini. However, if the complementary restriction sites used to fragment the DNA are not present in the cloning vector, the ends of the DNA molecules may be enzymatically modified. Alternatively, any site desired may be produced by ligating nucleotide sequences (linkers) onto the DNA termini; these ligated linkers may comprise specific chemically synthesized oligonucleotides encoding restriction endonuclease recognition sequences. Recombinant molecules can be introduced into host cells via transformation, transfection, infection, electroporation, etc., so that many copies of the gene sequence are generated. Preferably, the cloned gene is contained on a shuttle vector plasmid, which provides for expansion in a cloning cell, e.g., E. coli, and facile purification for subsequent insertion into an appropriate expression cell line, if such is desired. For example, a shuttle vector, which is a vector that can replicate in more than one type of organism, can be prepared for replication in both E. coli and Saccharomyces cerevisiae by linking sequences from an E. coli plasmid with sequences from the yeast 2μ plasmid.

In an alternative method, the desired gene may be identified and isolated after insertion into a suitable cloning vector in a “shot gun” approach. Enrichment for the desired gene, for example, by size fractionation, can be done before insertion into the cloning vector.

### Expression of RNA Polymerase

The nucleotide sequence coding for RNA polymerase, a fragment of RNA polymerase or a derivative or analog thereof, including a functionally active derivative, such as a chimeric protein, thereof, can be inserted into an appropriate expression vector, i.e., a vector which contains the necessary elements for the transcription and translation of the inserted protein-coding sequence. Such elements are termed herein a “promoter.” Thus, the nucleic acid encoding a RNA polymerase of the invention or a fragment thereof is operationally associated with a promoter in an expression vector of the invention. Both cDNA and genomic sequences can be cloned and expressed under control of such regulatory sequences. An expression vector also preferably includes a replication origin.

The necessary transcriptional and translational signals can be provided on a recombinant expression vector, or they may be supplied by the native gene encoding RNA polymerase and/or its flanking regions.

Potential host-vector systems include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

A recombinant RNA polymerase protein of the invention, or RNA polymerase fragment, derivative, chimeric construct, or analog thereof, may be expressed chromosomally, after integration of the coding sequence by recombination. In this regard, any of a number of amplification systems may be used to achieve high levels of stable gene expression [See Sambrook and Russell, 2001, supra].

The cell containing the recombinant vector comprising the nucleic acid encoding RNA polymerase is cultured in an appropriate cell culture medium under conditions that provide for expression of RNA polymerase by the cell.

Any of the methods previously described for the insertion of DNA fragments into a cloning vector may be used to construct expression vectors containing a gene consisting of appropriate transcriptional/translational control signals and the protein coding sequences. These methods may include in vitro recombinant DNA and synthetic techniques and in vivo recombination (genetic recombination).

Expression of RNA polymerase may be controlled by any promoter/enhancer element known in the art, but these regulatory elements must be functional in the host selected for expression. Promoters that may be used to control RNA polymerase gene expression are well known in the art including prokaryotic expression vectors such as the β-lactamase promoter [Villa-Kamaroff, et al., Proc. Natl. Acad. Sci. U.S.A., 75:3727-3731 (1978)], or the tac promoter [DeBoer, et al., Proc. Natl. Acad. Sci. U.S.A., 80:21-25 (1983)].

Expression vectors containing a nucleic acid encoding an RNA polymerase of the invention can be identified by a number of means including four general approaches: (a) PCR amplification of the desired plasmid DNA or specific mRNA, (b) nucleic acid hybridization, (c) presence or absence of selection marker gene functions, and (d) expression of inserted sequences. In the first approach, the nucleic acids can be amplified by PCR to provide for detection of the amplified product. In the second approach, the presence of a foreign gene inserted in an expression vector can be detected by nucleic acid hybridization using probes comprising sequences that are homologous to an inserted marker gene. In the third approach, the recombinant vector/host system can be identified and selected based upon the presence or absence of certain “selection marker” gene functions (e.g., β-galactosidase activity, thymidine kinase activity, resistance to antibiotics, transformation phenotype, occlusion body formation in baculovirus, etc.) caused by the insertion of foreign genes in the vector. In another example, if the nucleic acid encoding RNA polymerase is inserted within the “selection marker” gene sequence of the vector, recombinants containing the RNA polymerase insert can be identified by the absence of the selection marker gene function. In the fourth approach, recombinant expression vectors can be identified by assaying for the activity, biochemical, or immunological characteristics of the RNA polymerase expressed by the recombinant, provided that the expressed protein assumes a functionally active conformation.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX [Smith et al., Gene, 67:31-40 (1988)], pMB9 and their derivatives, plasmids such as RP4; phage DNAS, e.g., the numerous derivatives of phage λ, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

For example, in a baculovirus expression systems, both non-fusion transfer vectors, such as but not limited to pVL941 (BamH1 cloning site; Summers), pVL1393 (BamH1, SmaI, XbaI, EcoR1, NotI, XmaIII, BglII, and PstI cloning site; Invitrogen), pVL1392 (BglII, PstI, NotI, XmaIII, EcoRI, XbaI, SmaI, and BamH1 cloning site; Summers and Invitrogen), and pBlueBacIII (BamH1, BglII, PstI, NcoI, and HindIII cloning site, with blue/white recombinant screening possible; Invitrogen), and fusion transfer vectors, such as but not limited to pAc700 (BamH1 and KpnI cloning site, in which the BamH1 recognition site begins with the initiation codon; Summers), pAc701 and pAc702 (same as pAc700, with different reading frames), pAc360 (BamH1 cloning site 36 base pairs downstream of a polyhedrin initiation codon; Invitrogen(195)), and pBlueBacHisA, B, C (three different reading frames, with BamH1, BglII, PstI, NcoI, and HindIII cloning site, an N-terminal peptide for ProBond purification, and blue/white recombinant screening of plaques; Invitrogen (220)) can be used.

Mammalian expression vectors contemplated for use in the invention include vectors with inducible promoters, such as the dihydrofolate reductase (DHFR) promoter, e.g., any expression vector with a DHFR expression vector, or a DHFR/methotrexate co-amplification vector, such as pED (PstI, SalI, SbaI, SmaI, and EcoRI cloning site, with the vector expressing both the cloned gene and DHFR; see Kaufman, Current Protocols in Molecular Biology, 16.12 (1991). Alternatively, a glutamine synthetase/methionine sulfoximine co-amplification vector, such as pEE14 (HindIII, XbaI, SmaI, SbaI, EcoRI, and BclI cloning site, in which the vector expresses glutamine synthase and the cloned gene; Celltech). In another embodiment, a vector that directs episomal expression under control of Epstein Barr Virus (EBV) can be used, such as pREP4 (BamH1, SfiI, XhoI, NotI, NheI, HindIII, NheI, PvuII, and KpnI cloning site, constitutive RSV-LTR promoter, hygromycin selectable marker; Invitrogen), pCEP4 (BamH1, SfiI, XhoI, NotI, NheI, HindIII, NheI, PvuII, and KpnI cloning site, constitutive hCMV immediate early gene, hygromycin selectable marker; Invitrogen), pMEP4 (KpnI, PvuI, NheI, HindIII, NotI, XhoI, SfiI, BamH1 cloning site, inducible methallothionein IIa gene promoter, hygromycin selectable marker: Invitrogen), pREP8 (BamH1, XhoI, NotI, HindIII, NheI, and KpnI cloning site, RSV-LTR promoter, histidinol selectable marker; Invitrogen), pREP9 (KpnI, NheI, HindIII, NotI, XhoI, SfiI, and BamHI cloning site, RSV-LTR promoter, G418 selectable marker; Invitrogen), and pEBVHis (RSV-LTR promoter, hygromycin selectable marker, N-terminal peptide purifiable via ProBond resin and cleaved by enterokinase; Invitrogen). Selectable mammalian expression vectors for use in the invention include pRc/CMV (HindIII, BstXI, NotI, SbaI, and ApaI cloning site, G418 selection; Invitrogen), pRc/RSV (HindIII, SpeI, BstXI, NotI, XbaI cloning site, G418 selection; Invitrogen), and others. Vaccinia virus mammalian expression vectors (see, Kaufman, 1991, supra) for use according to the invention include but are not limited to pSC11 (SmaI cloning site, TK- and β-gal selection), pMJ601 (SalI, SmaI, AflI, NarI, BspMII, BamHI, ApaI, NheI, SacII, KpnI, and HindIII cloning site; TK- and β-gal selection), and pTKgptF1S (EcoRI, PstI, SalI, AccI, HindII, SbaI, BamHI, and Hpa cloning site, TK or XPRT selection).

Yeast expression systems can also be used according to the invention to express the bacterial RNA polymerase. For example, the non-fusion pYES2 vector (XbaI, SphI, ShoI, NotI, GstXI, EcoRI, BstXI, BamH1, SacI, Kpn1, and HindIII cloning sit; Invitrogen) or the fusion pYESHisA, B, C (XbaI, SphI, ShoI, NotI, BstXI, EcoRI, BamH1, SacI, KpnI, and HindIII cloning site, N-terminal peptide purified with ProBond resin and cleaved with enterokinase; Invitrogen), to mention just two, can be employed according to the invention.

Once a particular recombinant DNA molecule is identified and isolated, several methods known in the art may be used to propagate it. Once a suitable host system and growth conditions are established, recombinant expression vectors can be propagated and prepared in quantity. As previously explained, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

Vectors are introduced into the desired host cells by methods known in the art, e.g., transfection, electroporation, microinjection, transduction, cell fusion, DEAE dextran, calcium phosphate precipitation, lipofection (lysosome fusion), use of a gene gun, or a DNA vector transporter [see, e.g., Wu et al., J. Biol. Chem., 267:963-967 (1992); Wu and Wu, J. Biol. Chem., 263:14621-14624 (1988); Hartmut et al., Canadian Patent Application No. 2,012,311, filed Mar. 15, 1990).

### Peptide Synthesis

Synthetic polypeptides, prepared using the well known techniques of solid phase, liquid phase, or peptide condensation techniques, or any combination thereof, can include natural and unnatural amino acids. Amino acids used for peptide synthesis may be standard Boc (N^{α}-amino protected N^{α}-t-butyloxycarbonyl) amino acid resin with the standard deprotecting, neutralization, coupling and wash protocols of the original solid phase procedure of Merrifield [J. Am. Chem. Soc., 85:2149-2154 (1963)], or the base-labile N^{α}-amino protected 9-fluorenylmethoxycarbonyl (Fmoc) amino acids first described by Carpino and Han [J. Org. Chem., 37:3403-3409 (1972)]. Both Fmoc and Boc N^{α}-amino protected amino acids can be obtained from Fluka, Bachem, Advanced Chemtech, Sigma, Cambridge Research Biochemical, Bachem, or Peninsula Labs or other chemical companies familiar to those who practice this art. In addition, the method of the invention can be used with other N^{α}-protecting groups that are familiar to those skilled in this art. Solid phase peptide synthesis may be accomplished by techniques familiar to those in the art and provided, [e.g., Stewart and Young, Solid Phase Synthesis, Second Edition, Pierce Chemical Co., Rockford, Ill. (1984); Fields and Noble, Int. J. Pept. Protein Res. 35:161-214 (1990)], or using automated synthesizers, such as sold by ABS. Thus, polypeptides of the invention may comprise D-amino acids, a combination of D- and L-amino acids, and various “designer” amino acids (e.g., β-methyl amino acids, Cα-methyl amino acids, and Nα-methyl amino acids, etc.) to convey special properties. Synthetic amino acids include ornithine for lysine, fluorophenylalanine for phenylalanine, and norleucine for leucine or isoleucine. Additionally, by assigning specific amino acids at specific coupling steps, α-helices, β turns, β sheets, γ-tums, and cyclic peptides can be generated.

### Isolation and Crystallization of the Bacterial RNA Polymerase

The present invention provides a crystal of the Rif-RNAP complex that can be effectively diffract X-rays for the determination of the atomic coordinates of the Rif-RNAP to a resolution of better than 5.0 Angstroms and preferably to a resolution equal to or better than 3.5 Angstroms. The RNA polymerase can be expressed either as described above or as described in U.S. Ser. No. 09/396,651, Filed Sep. 15, 1999, the contents of which are hereby incorporated by reference in their entireties. Of course, the specific Rif-RNAP complex provided herein serves only as example, since the crystallization process can tolerate a broad range of active RNA polymerases and inhibitors. Therefore, any person with skill in the art of protein crystallization having the present teachings and without undue experimentation could crystallize a large number of alternative forms of the RNA polymerases from a variety of RNA polymerase fragments, or alternatively using a full length RNA polymerase from a related source and then allow the RNA polymerase to bind rifampicin and/or other RNAP binding partners (e.g., inhibitors) as described below. As mentioned above, an RNA polymerase having conservative substitutions in its amino acid sequence are also included in the invention, including a selenomethionine substituted form.

Crystals of the RNA polymerase can be grown by a number of techniques including batch crystallization, vapor diffusion (either by sitting drop or hanging drop) and by microdialysis. Seeding of the crystals in some instances is required to obtain X-ray quality crystals. Standard micro and/or macro seeding of crystals may therefore be used.

The crystals of the RNA polymerase can be grown alone or co-crystallized with a binding partner such as rifampicin. If the crystals are grown alone they can be subsequently soaked in a stabilization buffer with an RNAP binding partner such as rifampicin (0.1 mM rifampicin was added in the Example below). The RNAP/RNAP-binding partner are preferably incubated in the stabilization buffer for at least twelve hours. An exemplary stabilization buffer contains between 1.7-2.3 M (NH₄)₂SO₄, 0.02-1 M Tris-HCl, pH 6.5-8.5, and approximately 20 mM MgCl₂.

The crystals are then prepared for cryo-crystallography by soaking the RNAP/RNAP-binding partner complex in a stabilization buffer (e.g., 2 M (NH₄)₂SO₄, 0.1 M Tris-HCl, pH 8.0, and 20 mM MgCl₂ containing 50% (w/v) sucrose) before flash freezing.

Aside from the methodology exemplified below, alternative methods may also be used to characterize the crystals. For example, crystals can be characterized by using X-rays produced in a conventional source (such as a sealed tube or a rotating anode) or using a synchrotron source. Methods of characterization include, but are not limited to, precision photography, oscillation photography and diffractometer data collection. Selenium-Methionine may be used, or alternatively a mercury derivative dataset (e.g., using PCMB) could be used in place of the selenium-methionine derivatization.

Structural determinations can be performed by calculating Patterson maps using PHASES [Furey and Swaminathan, Methods Enzymol., 277:590-620 (1997)] for the ethyl-HgCl₂ and Ta₆Br₁₄ derivatives and using the Pb-derivative as native, for example. In the Example below, the native core RNAP structure [Zhang et al., Cell 98:811-824 (1999); U.S. Ser. No. 09/396,651, Filed Sep. 15, 1999, the contents of which are hereby incorporated by reference in their entireties] was used as a starting model for rigid body refinement and positional refinement against the observed amplitudes from the Rif-RNAP complex crystal (Fₒ^{Rif}:) using CNS [Adams et al., Proc. Natl. Acad. Sci. USA, 94:5018-5023 (1997)], yielding an initial R-factor of 0.354 (R_{free}=0.41, where the same set of reflections was set aside as was used for the R_{free} determination of the native structure) for data from 100-3.2 Å resolution. An initial Fourier difference map, calculated using |Fₒ^{Rif}−Fₒ^{nat}| amplitude coefficients and using phases calculated from the native core RNAP structure (Φ^{nat}) clearly revealed density for the rifampicin molecule (FIG. 3a). Multiple rounds of manual rebuilding against (2|Fₒ|−|F_{c}|) maps using O [Jones et al., Acta Cryst, A 47:110-119 (1991)], and refinement using CNS [Adams et al., Proc. Natl. Acad. Sci. USA, 94:5018-5023 (1997)] resulted in the current model (Table 1). At later stages of the refinement, the rifampicin X-ray crystal structure [Brufani et al., J. Molec.Biol. 87:409-435 (1974)] was placed into the difference density. Included in the model is the recently determined sequence of the Taq ω subunit modeled earlier as a polyalanine chain [Zhang et al., Cell 98:811-824 (1999); U.S. Ser. No. 09/396,651, Filed Sep. 15, 1999, the contents of which are hereby incorporated by reference in their entireties]. Absent from the model is a 300 amino acid, non-conserved domain inserted between conserved regions A and B of the β′ subunit [Zhang et al., Cell 98:811-824 (1999); U.S. Ser. No. 09/396,651, Filed Sep. 15, 1999, the contents of which are hereby incorporated by reference in their entireties].

### Protein-structure Based Design of Inhibitors of Bacterial RNA Polymerase

Once the three-dimensional structure of a crystal comprising a Rif-RNAP complex is determined, (e.g., see the coordinates in Table 2 below, in Appendix following the Sequence Listing) a potential modulator of RNA Polymerase, can be examined through the use of computer modeling using a docking program such as GRAM, DOCK, or AUTODOCK [Dunbrack et al., Folding & Design, 2:27-42 (1997)], to identify potential modulators of the RNA Polymerase. This procedure can include computer fitting of potential modulators to the RNA Polymerase to ascertain how well the shape and the chemical structure of the potential modulator will bind to either the individual bound subunits or to the RNA Polymerase [Bugg et al., Scientific American, December:92-98 (1993); West et al., TIPS, 16:67-74 (1995)]. Computer programs can also be employed to estimate the attraction, repulsion, and steric hindrance of the subunits with a modulator/inhibitor (e.g., the RNA Polymerase and a potential stabilizer).

Indeed, the shape of RNA polymerase resembles a crab-claw, with an internal groove or channel running along the full-length (between the claws). The molecule is about 150 Å long (from the back to the tips of the claws), 115 Å tall, and 110 Å wide (along the direction of the channel). The channel has many internal features, but the overall width is about 27 Å [see, U.S. Ser. No. 09/396,651, Filed Sep. 15, 1999, the contents of which are hereby incorporated by reference in their entireties].

As disclosed herein the three-dimensional structure demonstrates that rifampicin binds the Taq core RNAP with a close complementary fit in a pocket between two structural domains of the RNAP β subunit. Only small, local conformational changes of both the inhibitor and the protein is observed. The binding site is deep within the main RNAP channel, but the closest approach of the inhibitor to the RNAP active site Mg²⁺ is more than 12 Å.

Importantly, the structural information disclosed herein demonstrates that rifampicin inhibits RNA polymerase by physically blocking transcription elongation. This is in direct contrast with the modus operandi of a classical enzyme inhibitor which generally binds to the catalytic center or with a key transition state intermediate. Therefore, the effect of rifampicin depends only on its ability to bind tightly to a relatively non-conserved part of the structure, thereby disrupting a critical RNAP function. Thus, the structural information disclosed herein provides the impetus to investigate the binding of other unrelated small molecules to any of a variety of sites within the RNAP channel, which could also block transcription elongation. A preferred site is one that is critical for the transcriptional activity of bacterial RNA polymerase, but one that is not required by the corresponding mammalian enzyme.

Towards this end, generally the tighter the fit, the lower the steric hindrances, and the greater the attractive forces, the more potent the potential modulator (e.g., an inhibitor) since these properties are consistent with a tighter binding constant. Furthermore, the more specificity in the design of a potential drug the more likely that the drug will not interact as well with other proteins. This will minimize potential side-effects due to unwanted interactions with other proteins.

Initially alternative compounds known to bind bacterial RNA polymerase, including rifampicin analogs, can be systematically modified by computer modeling programs until one or more promising potential analogs are identified. In addition systematic modification of selected analogs can then be systematically modified by computer modeling programs until one or more potential analogs are identified. Such analysis has been shown to be effective in the development of HIV protease inhibitors [Lam et al., Science 263:380-384 (1994); Wlodawer et al., Ann. Rev. Biochem. 62:543-585 (1993); Appelt, Perspectives in Drug Discovery and Design 1:23-48 (1993); Erickson, Perspectives in Drug Discovery and Design 1: 109-128 (1993)]. Alternatively a potential modulator could be obtained by initially screening a random peptide library produced by recombinant bacteriophage for example, [Scott and Smith, Science, 249:386-390 (1990); Cwirla et al., Proc. Natl. Acad. Sci., 87:6378-6382 (1990); Devlin et al., Science, 249:404-406 (1990)]. A peptide selected in this manner would then be systematically modified by computer modeling programs as described above, and then treated analogously to a structural analog as described below.

Once a potential modulator/inhibitor is identified it can be either selected from a library of chemicals as are commercially available from most large chemical companies including Merck, GlaxoWelcome, Bristol Meyers Squib, Monsanto/Searle, Eli Lilly, Novartis and Pharmacia UpJohn, or alternatively the potential modulator may be synthesized de novo. The de novo synthesis of one or even a relatively small group of specific compounds is reasonable in the art of drug design. The potential modulator can be placed into a standard binding assay with RNA polymerase or an active fragment thereof, for example. The subunit fragments can be synthesized by either standard peptide synthesis described above, or generated through recombinant DNA technology or classical proteolysis. Alternatively the corresponding full-length proteins may be used in these assays.

For example, the β subunit can be attached to a solid support. Methods for placing the β subunit on the solid support are well known in the art and include such things as linking biotin to the β subunit and linking avidin to the solid support. The solid support can be washed to remove unreacted species. A solution of a labeled potential modulator (e.g., an inhibitor) can be contacted with the solid support. The solid support is washed again to remove the potential modulator not bound to the support. The amount of labeled potential modulator remaining with the solid support and thereby bound to the β subunit can be determined. Alternatively, or in addition, the dissociation constant between the labeled potential modulator and the β subunit, for example can be determined. Suitable labels for either the bacterial RNA polymerase subunit or the potential modulator are exemplified herein. In a particular embodiment, isothermal calorimetry can be used to determine the stability of the bacterial RNA polymerase in the absence and presence of the potential modulator.

In another embodiment, a Biacore machine can be used to determine the binding constant of the bacterial RNA polymerase to a DNA template in the presence and absence of the potential modulator. Alternatively, one or more of the bacterial RNA polymerase subunits can be immobilized on a sensor chip. The remaining subunits can then be contacted with (e.g., flowed over) the sensor chip to form the bacterial RNA polymerase.

In this case the dissociation constant for the bacterial RNA polymerase can be determined by monitoring changes in the refractive index with respect to time as buffer is passed over the chip [O'Shannessy et al. Anal. Biochem. 212:457-468 (1993); Schuster et al., Nature 365:343-347 (1993)]. Scatchard plots, for example, can be used in the analysis of the response functions using different concentrations of a particular subunit. Flowing a potential modulator at various concentrations over the bacterial RNA polymerase and monitoring the response function (e.g., the change in the refractive index with respect to time) allows the bacterial RNA polymerase dissociation constant to be determined in the presence of the potential modulator and thereby indicates whether the potential modulator is either an inhibitor, or an agonist of the bacterial RNA polymerase complex.

In another aspect of the present invention a potential modulator is assayed for its ability to inhibit the bacterial RNA polymerase. A modulator that inhibits the RNA polymerase can then be selected. In a particular embodiment, the effect of a potential modulator on the catalytic and/or transcriptional activity of bacterial RNA polymerase is determined. The potential modulator is then be added to a bacterial culture to ascertain its effect on bacterial proliferation. A potential modulator that inhibits bacterial proliferation can then be selected.

In a particular embodiment, the effect of the potential modulator on the catalytic and/or transcriptional activity of the bacterial RNA polymerase is determined (either independently, or subsequent to a binding assay as exemplified above). In one such embodiment, the rate of the DNA-dependent RNA transcription is determined. For such assays a labeled nucleotide could be used. This assay can be performed using a real-time assay e.g., with a fluorescent analog of a nucleotide. Alternatively, the determination can include the withdrawal of aliquots from the incubation mixture at defined intervals and subsequent placing of the aliquots on nitrocellulose paper or on gels. In a particular embodiment the potential modulator is selected when it is an inhibitor of the bacterial RNA polymerase.

One assay for RNA polymerase activity is a modification of the method of Burgess et al. [J. Biol. Chem., 244:6160 (1969)]

[See also http://www.worthington-biochem.com/manual/R/RNAP.html].

One unit incorporates one nanomole of UMP into acid insoluble products in 10 minutes at 37° C. under the assay conditions such as those listed below.

The suggested reagents are:

(a) 0.04 M Tris-HCl, pH 7.9, containing 0.01 M MgCl₂, 0.15 M KCl, and 0.5 mg/ml BSA;

(b) Nucleoside triphosphates (NTP) : 0.15 mM each of ATP, CTP, GTP, UTP; spiked with ³H-UTP 75000-150000 cpms/0.1 ml;

(c) 0.15 mg/ml calf thymus DNA;

(d) 10% cold perchloric acid; and

(e) 1% cold perchloric acid.

0.1-0.5 units of RNA polymerase in 5 μl-10 μl is used as the starting enzyme concentration.

The procedure is to add 0.1 ml Tris-HCl, 0.1 ml NTP and 0.1 ml DNA to a test tube for each sample or blank. At zero time enzyme (or buffer for blank) is added to each test tube, and the contents are then mixed and incubated at 37° C. for 10 minutes. 1 ml of 10% perchloric acid is added to the tubes to stop the reaction. The acid insoluble products can be collected by vacuum filtration through MELIPORE filter discs having a pore size of 0.45 u-10 u (or equivalent). The filters are then washed four times with 1% cold perchloric acid using 1 ml-3 ml for each wash. These filters are then placed in scintillation vials. 2 mls of methyl cellosolve are added to the scintillation vials to dissolve the filters. When the filters are completely dissolved (after about five minutes) 10 mls of scintillation fluid are added and the vials are counted in a scintillation counter.

For calculation of units of RNA polymerase/mg of protein the following equation can be used:

$$units \text{/} mg = \frac{{CPM}_{test} - {CPM}_{blank}}{{CPM}_{total} \times mg {protein}_{i n test}}$$

Alternative transcription assays can also be employed [see Example below, and Nudler et al., Science 265:793-796 (1994)]. One such assay comprises a core RNAP that can be incubated with a suitable σ subunit to form the holoenzyme. A potential modulator can then be added prior to, simultaneously with, subsequently to a promoter fragment (e.g., T7A1 as exemplified below). RNA synthesis is then initiated by the addition of a primer (e.g., a CpA primer) and the four nucleotide triphosphates (NTPs). The RNA synthesis in the presence and absence of the potential modulator is then quantified. In the Example below, a radioactive nucleotide was employed and the radioactive RNA products were analyzed on a 15% polyacrylamide sequencing gel. Alternatively, a fluorescent nucleotide analog can be used. Transcription reactions on a minimal scaffold system can be performed as shown in FIG. 6b below in the presence and the absence of the potential modulator [see also Korzheva et al., Science 289:619-625 (2000)].

When suitable potential modulators are identified, a supplemental crystal can be prepared which comprises the bacterial RNA polymerase and the potential modulator (see Example below). Preferably the crystal effectively diffracts X-rays for the determination of the atomic coordinates of the protein-ligand complex to a resolution of better than 5.0 Angstroms, more preferably equal to or better than 3.5 Angstroms. The three-dimensional structure of the supplemental crystal can be determined by Molecular Replacement Analysis. Molecular replacement involves using a known three-dimensional structure as a search model to determine the structure of a closely related molecule or protein-ligand complex in a new crystal form. The measured X-ray diffraction properties of the new crystal are compared with the search model structure to compute the position and orientation of the protein in the new crystal. Computer programs that can be used include: X-PLOR (see above), CNS, (Crystallography and NMR System, a next level of XPLOR), and AMORE [J. Navaza, Acta Crystallographics ASO, 157-163 (1994)]. Once the position and orientation are known an electron density map can be calculated using the search model to provide X-ray phases. Thereafter, the electron density is inspected for structural differences and the search model is modified to conform to the new structure. Using this approach, it is also possible to use the claimed crystal of the Rif-RNAP complex to solve the three-dimensional structures of other bacterial core RNA polymerases bound to rifampicin (and/or other inhibitors) having pre-ascertained amino acid sequences. Other computer programs that can be used to solve the structures of the bacterial RNA polymerase from other organisms include: QUANTA, CHARMM; INSIGHT; SYBYL; MACROMODE; and ICM.

A candidate drug can be selected by performing rational drug design with the three-dimensional structure determined for the supplemental crystal, preferably in conjunction with computer modeling discussed above. The candidate drug (e.g., a potential modulator of bacterial RNA polymerase) can then be assayed as exemplified above, or in situ. A candidate drug can be identified as a drug, for example, if it inhibits bacterial proliferation.

A potential inhibitor (e.g., a candidate drug) would be expected to interfere with bacterial growth. Therefore, an assay that can measure bacterial growth may be used to identify a candidate drug.

Methods of testing a potential bactericidal agent (e.g., the candidate drug) in an animal model are well known in the art, and can include standard bactericidal assays. The potential modulators can be administered by a variety of ways including topically, orally, subcutaneously, or intraperitoneally depending on the proposed use. Generally, at least two groups of animals are used in the assay, with at least one group being a control group which is administered the administration vehicle without the potential modulator.

For all of the drug screening assays described herein further refinements to the structure of the drug will generally be necessary and can be made by the successive iterations of any and/or all of the steps provided by the particular drug screening assay.

### Labels

Suitable labels include enzymes, fluorophores e.g., fluorescein isothiocyanate (FITC), phycoerythrin (PE), Texas red (TR), rhodamine, free or chelated lanthanide series salts, especially Eu³⁺, to name a few fluorophores and including fluorescent GTP and GDP analogs such as mantGTP and mantGDP, chromophores, radioisotopes, chelating agents, dyes, colloidal gold, latex particles, ligands (e.g., biotin), and chemiluminescent agents. When a control marker is employed, the same or different labels may be used for the test and control marker.

In the instance where a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized colorimetric, spectrophotometric, fluorospectrophotometric, amperometric or gasometric techniques known in the art.

Direct labels are one example of labels which can be used according to the present invention. A direct label has been defined as an entity, which in its natural state, is readily visible, either to the naked eye, or with the aid of an optical filter and/or applied stimulation, e.g. ultraviolet light to promote fluorescence. Among examples of colored labels, which can be used according to the present invention, include metallic sol particles, for example, gold sol particles such as those described by Leuvering (U.S. Pat. No. 4,313,734); dye sole particles such as described by Gribnau et al. (U.S. Pat. No. 4,373,932) and May et al. (WO 88/08534); dyed latex such as described by May, supra, Snyder (EP-A 0 280 559 and 0 281 327); or dyes encapsulated in liposomes as described by Campbell et al. (U.S. Pat. No. 4,703,017) Other direct labels include a radionucleotide, a luminescent moiety, or a fluorescent moiety including as a modified/fusion chimera of green fluorescent protein (as described in U.S. Pat. No. 5,625,048 filed Apr. 29, 1997, and WO 97/26333, published Jul. 24, 1997, the disclosures of each are hereby incorporated by reference herein in their entireties). In addition to these direct labeling devices, indirect labels comprising enzymes can also be used according to the present invention. Various types of enzyme linked immunoassays are well known in the art, for example, alkaline phosphatase and horseradish peroxidase, lysozyme, glucose-6-phosphate dehydrogenase, lactate dehydrogenase, urease, these and others have been discussed in detail by Eva Engvall in Enzyme Immunoassay ELISA and EMIT in Methods in Enzymology, 70:419-439 (1980) and in U.S. Pat. No. 4,857,453.

Suitable enzymes include, but are not limited to, alkaline phosphatase and horseradish peroxidase. Other labels for use in the invention include magnetic beads or magnetic resonance imaging labels.

In another embodiment, a phosphorylation site can be created on an antibody of the invention for labeling with ³²P, e.g., as described in European Patent No. 0372707 (application No. 89311108.8) by Sidney Pestka, or U.S. Pat. No. 5,459,240, issued Oct. 17, 1995 to Foxwell et al.

As exemplified herein, proteins, including antibodies, can be labeled by metabolic labeling. Metabolic labeling occurs during in vitro incubation of the cells that express the protein in the presence of culture medium supplemented with a metabolic label, such as [³⁵S]-methionine or [³²P]-orthophosphate. In addition to metabolic (or biosynthetic) labeling with [³⁵S]-methionine, the invention further contemplates labeling with [¹⁴C]-amino acids and [³H]-amino acids (with the tritium substituted at non-labile positions).

### Three-Dimensional Representation of the Structure of the Rif-RNAP Complex

In addition, the present invention provides a computer that comprises a representation of the RNAP-RNAP binding partner complex (e.g., the Rif-RNAP complex) in computer memory that can be used to screen for compounds that will or are likely to inhibit RNAP. In a related embodiment, the computer can be used in the design of altered RNAPs that have either enhanced, or alternatively diminished RNA polymerase activity. Preferably, the computer comprises portions of and/or all of the information contained in Table 2. In a particular embodiment, the computer comprises: (i) a machine-readable data storage material encoded with machine-readable data, (ii) a working memory for storing instructions for processing the machine readable data, (iii) a central processing unit coupled to the working memory and the machine-readable data storage material for processing the machine-readable data into a three-dimensional representation, and (iv) a display coupled to the central processing unit for displaying the three-dimensional representation.

Thus the machine-readable data storage medium comprises a data storage material encoded with machine readable data which can comprise portions and/or all of the structural information contained in Table 2. One embodiment for manipulating and displaying the structural data provided by the present invention is schematically depicted in FIG. 7. As depicted, the System 1, includes a computer 2 comprising a central processing unit (“CPU”) 3, a working memory 4 which may be random-access memory or “core” memory, mass storage memory 5 (e.g., one or more disk or CD-ROM drives), a display terminal 6 (e.g., a cathode-ray tube), one or more keyboards 7, one or more input lines 10, and one or more output lines 20, all of which are interconnected by a conventional bidirectional system bus 30.

Input hardware 12, coupled to the computer 2 by input lines 10, may be implemented in a variety of ways. Machine-readable data may be inputted via the use of one or more modems 14 connected by a telephone line or dedicated data line 16. Alternatively or additionally, the input hardware 12 may comprise CD-ROM or disk drives 5. In conjunction with the display terminal 6, the keyboard 7 may also be used as an input device. Output hardware 22, coupled to computer 2 by output lines 20, may similarly be implemented by conventional devices. Output hardware 22 may include a display terminal 6 for displaying the three dimensional data. Output hardware might also include a printer 24, so that a hard copy output may be produced, or a disk drive 5, to store system output for later use, see also U.S. Pat. No. 5,978,740, Issued Nov. 2, 1999, the contents of which are hereby incorporated by reference in their entireties.

In operation, the CPU 3 (i) coordinates the use of the various input and output devices 12 and 22; (ii) coordinates data accesses from mass storage 5 and accesses to and from working memory 4; and (iii) determines the sequence of data processing steps. Any of a number of programs may be used to process the machine-readable data of this invention.

The present invention may be better understood by reference to the following non-limiting Example, which is provided as exemplary of the invention. The following example is presented in order to more fully illustrate the preferred embodiments of the invention. It should in no way be construed, however, as limiting the broad scope of the invention.

### EXAMPLE

### Structural Mechanism for Rifampicin Inhibition of bacterial RNA Polymerase

### Introduction

High-resolution structural studies of the Rif-RNAP complex should lead to insights into rifampicin binding, the mechanism of inhibition, and also the mechanism by which mutations lead to Rif^{R}. These structural studies will also shed light on the transcription mechanism itself, as well as provide the basis for the development of drugs that selectively inhibit bacterial RNAPs, but are less prone than rifampicin to lead bacterial mutations/substitutions of single amino acids that give rise to resistance. Indeed, the recent determination of the crystal structure of core RNAP from Thermus aquaticus (Taq) [Zhang et al., Cell 98:811-824 (1999); U.S. Ser. No. 09/396,651, Filed Sep. 15, 1999, the contents of which are hereby incorporated by reference in their entireties] has opened the door to further studies of RNAP structure, function, and interactions with substrates, ligands, and inhibitors.

To further provide a more detailed framework to interpret the existing genetic, biochemical, and biophysical information, as well as to guide further studies aimed at understanding the transcription process and its regulation, the three-dimensional structure of a bacterial core RNAP complexed with rifampicin was determined by X-ray crystallography at 3.3 Å resolution as detailed below. The structure explains the effects of rifampicin on RNAP function. In combination with a model of the ternary transcription complex and biochemical experiments, the data indicate that the predominant effect of rifampicin on RNAP function is to directly block the path of the elongating RNA transcript at the 5′-end when the transcript becomes either 2 or 3 nucleotides in length.

### Methods

Purification and crystallization: Native Taq core RNAP was purified and crystallized as described previously [Zhang et al., Cell 98:811-824 (1999); U.S. Ser. No. 09/396,651, Filed Sep. 15, 1999, the contents of which are hereby incorporated by reference in their entireties]. Crystals were subsequently soaked in stabilization solution [2 M (NH₄)₂SO₄, 0.1 M Tris-HCl, pH 8.0, and 20 mM MgCl₂] with 0.1 mM rifampicin for at least 12 hours. The crystals were then prepared for cryo-crystallography by soaking in stabilization solution containing 50% (w/v) sucrose for 30 minutes before flash freezing in liquid nitrogen. Diffraction data was collected at the APS beamline SBC 19ID using 0.3° oscillations, and processed using DENZO and SCALEPACK [Otwinowski, Isomorphous Replacement and Anomalous Scattering (eds. Wolf, Evans and Leslie) Science and Engineering Research Council, Daresbury Laboratory, Daresbury, UK, (1991)].

In short, the preparative procedure for T. aquaticus core RNAP is similar to the preparation of E. coli core RNAP [Polyakov et al., Cell, 83:365-373 (1995)]. Briefly, approximately 200 g wet cell paste is thawed and lysed using a continuous-flow French press. After a low-speed spin, the soluble fraction is precipitated with 0.6% Polymin-P. RNAP is eluted from the Polymin-P pellet with TGED buffer (10 mM Tris-HCl, pH 8, 5% glycerol, 1 mM EDTA, 1 mM DTT) plus 1 M NaCl, then precipitated by adding 33%(g/v) ammonium sulfate. The pellet is resuspended and loaded onto a 50 ml column of heparin-SEPHAROSE FF (Pharmacia) equilibrated with TGED buffer plus 0.2 M NaCl. The RNAP is eluted from the column with TGED buffer plus 0.6 M NaCl. The RNAP was again precipitated with ammonium sulfate, then resuspended and loaded on a SUPERDEX-200 gel filtration column equilibrated with TGED buffer plus 0.5 M NaCl. Fractions containing RNAP were pooled and loaded onto a MONO-Q (Pharmacia) ion-exchange column equilibrated with TGED buffer plus 0.1 M NaCl. The protein was eluted with a gradient from 0.1 to 0.5 M NaCl. The RNAP peak eluted at around 0.3 M NaCl. The RNAP was concentrated using a centrifugal filter, then loaded onto an SP SEPHAROSE (Pharmacia) column equilibrated in TGED buffer plus 0.1 M NaCl. After loading, the column was incubated at 4° C. for at least 10 hours, then pure RNAP was eluted with a 0.1 to 0.5 M NaCl gradient (core RNAP elutes at around 0.3 M NaCl). 200 g wet cell paste typically yielded 15 mg of core RNAP, which was more than 99% pure as judged from overloaded, Coomassie-stained SDS gels. This sample is ready for crystallization.

Crystals of T. aquaticus core RNAP were grown by vapor diffusion. 10 μl of T. aquaticus core RNAP (17 mg/ml) was mixed with the same volume of a solution containing 40-45% saturated (NH₄)₂SO₄, 0.1 M Tris-HCl, pH 8.0, and 20 mM MgCl₂, and incubated as a hanging drop over the same solution. Crystals grow in 2-3 weeks to typical dimensions of 0.15 mm×0.15 mm×0.4 mm at room temperature. For cryo-crystallography, the crystals are pre-soaked in stabilization solution (same as the crystallization solution except with 50% saturated ammonium sulfate). The crystals are then soaked in stabilization solution containing 50% (g/v) sucrose for about 30 minutes before flash freezing. The frozen crystals diffract to 5.0 Å from an in-house X-ray generator. Spots can sometimes be observed, in one direction, to 2.7 Å resolution at synchrotron beamlines. Diffraction data was processed using DENZO and SCALEPACK [Otwinowski, Isomorphous Replacement and Anomalous Scattering (eds. Wolf, Evans and Leslie) Science and Engineering Research Council, Daresbury Laboratory, Daresbury, UK, (1991)].

Selenomethionyl core RNAP was prepared and crystallized using the same procedures from T. aquaticus cells grown in minimal media (culture medium 162) [Degryse et al., Arch. Microbiol., 117:189-196 (1978)]. Cells were induced to incorporate selenomethionine by suppression of methionine biosynthesis [Doublie, Methods Enzymol., 276:523-530 (1997)].

Structure Determination: The native core RNAP structure [Zhang et al., Cell 98:811-824 (1999); U.S. Ser. No. 09/396,651, Filed Sep. 15, 1999, the contents of which are hereby incorporated by reference in their entireties] was used as a starting model for rigid body refinement and positional refinement against the observed amplitudes from the Rif-RNAP complex crystal (Fₒ^{Rif}:) using CNS [Adams et al., Proc. Natl. Acad. Sci. USA, 94:5018-5023 (1997)], yielding an initial R-factor of 0.354 (R_{free}=0.41, where the same set of reflections was set aside as was used for the R_{free} determination of the native structure) for data from 100-3.2 Å resolution. An initial Fourier difference map, calculated using |F_{Rif}−Fₒ^{nat}| amplitude coefficients and using phases calculated from the native core RNAP structure (Φ^{nat}) clearly revealed density for the Rif molecule (FIG. 3a). Multiple rounds of manual rebuilding against (2|Fₒ|−|Fₒ|) maps using O [Jones et al., Acta Cryst, A 47:110-119 (1991)], and refinement using CNS [Adams et al., Proc. Natl. Acad. Sci. USA, 94:5018-5023 (1997)] resulted in the current model (Table 1). At later stages of the refinement, the Rif X-ray crystal structure [Brufani et al., J. Molec.Biol. 87:409-435 (1974)] was easily placed into the difference density. Included in the model is the recently determined sequence of the Taq ω subunit modeled earlier as a polyalanine chain [Zhang et al., Cell 98:811-824 (1999); U.S. Ser. No. 09/396,651, Filed Sep. 15, 1999, the contents of which are hereby incorporated by reference in their entireties]. Absent from the model is a 300 amino acid, non-conserved domain inserted between conserved regions A and B of the β′ subunit [Zhang et al., Cell 98:811-824 (1999); U.S. Ser. No. 09/396,651, Filed Sep. 15, 1999, the contents of which are hereby incorporated by reference in their entireties].

Assays: Taq cells were tested for sensitivity to rifampicin on solid media. Plates containing 3% bactoagar and ⅕ dilution of Luria broth were poured with and without 50 μg/ml of rifampicin. Cells from frozen stock were then streaked onto plates and incubated at 65° C. for 2 days and assessed for growth.

The transcription assay comparing rifampicin inhibition of E.coli and Taq RNAPs (FIG. 2a) was performed as previously described [Nudler et al., Science 265:793-796 (1994)]. Briefly, 0.1 pmol of purified Taq core RNAP [Zhang et al., Cell 98:811-824 (1999); U.S. Ser. No. 09/396,651, Filed Sep. 15, 1999, the contents of which are hereby incorporated by reference in their entireties] was incubated with Taq σ^{A} in 20 μl of transcription buffer (40 mM Tris-HCl, pH 7.9, 40 mM KCl, 5 mM MgCl₂) for 15 minutes at 37° C. to form holoenzyme. Rifampicin was added to the final concentrations indicated in FIG. 2a and incubated another 5 minutes at 37 ° C., followed by the addition of 0.15 pmol of T7A1 promoter fragment and incubation for 5 minutes at 37° C. RNA synthesis was initiated by the addition of CpA primer (100 μM), NTPs (25 μM each), and a-[³²P]UTP (0.3 μM), and the reaction was stopped after incubation for 10 minutes at 37° C. The assay for E.coli RNAP holoenzyme was the same except the CpA primer was added to a concentration of 10 μM. Radioactive RNA products were analyzed on a 15% polyacrylamide sequencing gel.

Assays for extension of the Rif-nucleotide compounds (FIG. 2c-2d) were carried out as described [Mustaev et al.,Proc.Nat.Acad.Sci. USA 91:12036-12040 (1994)] with minor modifications. After binary complex formation, transcription reactions were started by the addition 10 μM Rif-(CH₂)ₙ-A compound, with the ‘n’ indicated in FIG. 2c-2d, and α-[³²P]UTP (0.3 μM). The reactions were incubated for 2 minutes at room temperature for E.coli RNAP and 3 minutes at 55° C. for Taq. Under these conditions, the reaction was not complete, and the yield of the Rif-(CH₂)ₙ-ApU depended on the linker length. Radioactive RNA products were analyzed on a 23% polyacrylamide sequencing gel.

Transcription reactions on the minimal scaffold system shown (FIG. 6b) were performed as described [Korzheva et al., Science 289:619-625 (2000)] with minor modifications. The RNA and DNA components of the scaffold (100 pmol of each) were mixed in 100 μl of transcription buffer at 45° C. and the mixture was allowed to cool to room temperature over 30 minutes. RNAP/scaffold complexes were formed by incubation of the annealed scaffold (10 pmol) with a molar equivalent of core RNAP (either E.coli or Taq) which was preincubated with rifampicin (100 μM for E.coli, 200 μM for Taq) for 10 minutes to form the RNAP/scaffold complex. Extension of the RNA oligonucleotide was assayed by the addition of α-[³²P]CTP (0.3 μM) and a 5 minute incubation at room temperature. In FIG. 6b, lanes 1-5 and 16-20, RNAP was preincubated with rifampicin (100 μM for E. coli RNAP, 200 μM for Taq) for 10 minutes. In lanes 6-10 and 21-25, the RNAP/scaffold complexes formed in the absence of rifampicin were incubated with rifampicin (concentrations as above) for 10 minutes. Finally, in lanes 11-15 and 26-30, the RNAP or RNAP/scaffold complex was not exposed to rifampicin. Radioactive RNA products were analyzed on a 23% polyacrylamide sequencing gel.

### Results

Rifampicin Inhibition of Taq RNAP: From a biochemical perspective, the interaction of rifampicin (Rif) with RNAP has been extensively characterized using E. coli RNAP, which served as a prototype for bacterial pathogens [Drancourt and Raoult, Antimicr.AgentsChemother. 43:2400-2403 (1999); Heep et al., Antimicr.AgentsChemother. 44:1075-1077 (1999); Honore et al., Molec.Microbiol. 7:207-214 (1993); Morse et al., J.Clin.Microbiol, 37:2913-2929 (1999); Nolte, J.Antimicrob. Chemother. 39:747-755(1997); Padayachee and Klugman, Antimicr.AgentsChemother. 43:2361-2365 (1999); Ramaswamy and Musser, Tubercle and Lung Disease 79:3-29 (1998); Wichelhaus et al., Antimicr.AgentsChemother. 43:2813-2816(1999)]. The inhibition of Taq RNAP by rifampicin was therefore investigated to assess this system as a structural model for Rif-RNAP interactions. Sequence comparisons in the four distinct regions of rpoB which harbor Rif^{R} mutations indicate a very high level of conservation among prokaryotes. Between E. coli, Taq, and M. tuberculosis, the sequences are 91% identical over 60 residues (93% conserved), explaining the broad spectrum of rifampicin activity. Nevertheless, among the 23 positions with single amino-acid substitutions that give rise to Rif^{R} in either E. coli or M. tuberculosis, 5 of these positions (Taq β 387, 395, 398, 453, and 566; the Taq numbering is used throughout this application unless otherwise specified) are substituted in Taq (FIG. 1). In contrast, there is a relatively low level of conservation between prokaryotes and eukaryotes within these regions (FIG. 1), explaining the lack of rifampicin activity against eukaryotic RNAPs and eukaryotic cells.

A plate assay (see Methods above) was used to show that Taq cells were unable to grow on media supplemented with 50 μg/ml rifampicin. For in vitro studies, Taq RNAP holoenzyme was reconstituted using Taq core RNAP purified from Taq cells [Zhang et al., Cell 98:811-824 (1999); U.S. Ser. No. 09/396,651, Filed Sep. 15, 1999, the contents of which are hereby incorporated by reference in their entireties] and recombinant Taq σ^{A} (overexpressed and purified from E. coli). The enzyme initiated, elongated, and terminated transcripts efficiently from a template containing the T7A1 promoter and the tR2 intrinsic terminator (FIG. 2a) [Nudler et al., J.Molec.Biol. 288:1-12(1994)] at 37° C. using the dinucleotide CpA as the initating primer. The major RNA products, a trimeric abortive transcript (CpApU), a 105 nucleotide terminated transcript (Term), and a 127 nucleotide runoff transcript (Run off), were the same as those produced by E. coli RNAP (FIG. 2a, lanes 1 and 8). Since E. coli σ⁷⁰ is totally inactive when combined with Taq core RNAP in this assay, the possibility of trace contamination with E. coli σ⁷⁰ does not affect the conclusions from this assay for Taq RNAP. Quantitatively, the two RNAPs responded very differently to rifampicin, the Ki (estimated from the rifampicin concentration where the production of long transcripts was inhibited by 50%) for E. coli RNAP was about 0.1 μM, while for Taq RNAP it was about 10 μM, a 100-fold difference in sensitivity. Qualitatively, however, both RNAPs responded the same way, with an increase in the production of the trimeric product and a concurrent precipitous drop in the production of the long transcripts (FIG. 2a).

Mustaev et al., [Proc.Nat.Acad.Sci. USA 91:12036-12040 (1994)] used chimeric Rif-nucleotide compounds to measure the distance between the initiating nucleotide binding site (the i-site) and the Rif binding site. By varying the linker between the Rif and the nucleotide and testing for maximal transcription initiation activity, the optimal length was found that allowed binding of each moiety in its respective site. This experiment was used to compare the disposition of the Rif and i-sites in E. coli and Taq RNAP. In both cases, optimal initiation activity was observed when the linker comprised five -(CH2)- groups (FIGS. 2c-2d). Thus, in spite of the fact that Taq RNAP requires a 100-fold higher concentration of rifampicin for inhibition, Taq RNAP binds rifampicin and is inhibited through the same biochemical mechanism as E. coli RNAP, and the disposition of the Rif-site with respect to the universally conserved active site is identical. Therefore, Taq RNAP can serve as a model for rifampicin interactions with other RNAPs.

Rif-RNAP Structure Determination and Refinement: Tetragonal crystals of Taq core RNAP [Zhang et al., Cell 98:811-824 (1999); U.S. Ser. No. 09/396,651, Filed Sep. 15, 1999, the contents of which are hereby incorporated by reference in their entireties] were incubated overnight in stabilization buffer with 0.1 mM rifampicin, followed by a 30 minute soak in cryo-solution (without rifampicin) before flash freezing. During this procedure, the crystals took on a deep orange color, confirming the binding of rifampicin. The same results were obtained with co-crystals grown in the presence of 0.1 mM rifampicin, suggesting that rifampicin binding causes few if any conformational changes in the RNAP.

The Taq core RNAP:Rif crystals were isomorphous with the native Taq core RNAP crystals [Zhang et al., Cell 98:811-824 (1999); U.S. Ser. No. 09/396,651, Filed Sep. 15, 1999, the contents of which are hereby incorporated by reference in their entireties]. Strong electron density was observed in difference Fourier maps for the rifampicin (FIG. 3a), which occupies a shallow pocket between β structural domains 3 and 4 (FIG. 3b) that is surrounded by the known Rif^{R} mutations (FIG. 1) [Zhang et al., Cell 98:811-824 (1999); U.S. Ser. No. 09/396,651, Filed Sep. 15, 1999, the contents of which are hereby incorporated by reference in their entireties]. The electron difference density also indicated shifts and/or ordering of several β residues interacting directly with rifampicin, including Q390, L391, Q393, D396, H406, R409, and L413 (FIG. 4). Only very small shifts in localized regions of the protein backbone were indicated.

The rifampicin X-ray crystal structure [Brufani et al., J. Molec.Biol. 87:409-435 (1974)] was easily placed into the difference density. Subsequent refinements resulted in only small shifts of the ansa chain (FIG. 3c) to better fit the density. Multiple rounds of manual rebuilding against (2|Fₒ|−|Fₒ|) maps and refinement resulted in the current model (see Methods above and Table 1).

TABLE 1
CRYSTALLOGRAPHIC DATA AND STRUCTURAL MODEL
DIFFRACTION DATA

| Parameter | Total | Outer Shell |
|---|---|---|
| Resolution range (Å) | 30-3.3 | 3.42-3.3 |
| Rmerge¹ (%) | 7.7 | 34.4 |
| Completeness (%) | 86.1 | 71.7 |
| I/σI | 10.7 | 1.7 |
| No. of reflections | 75,420 | 6,173 |
| No. of unique obs. | 214,453 | 11,549 |

STRUCTURAL MODEL
Number of Residues

| Protein | | | | |
|---|---|---|---|---|
| Subunit² | Mr (kDa) | sequence | model | regions modeled |
| β′ | 170.7 | 1,525 | 1,139 | 3-31, 69-155 (poly-Ala), |
| | | | | 452-523, 536-1241, |
| | | | | 1250-1410, 1414-1497 |
| β | 124.4 | 1,119 | 1,114 | 2-1115 |
| αI | 34.9 | 313 | 223 | 6-228 |
| αII | 34.9 | 313 | 229 | 3-231 |
| ω | 11.6 | 99 | 98 | 1-98 |
| Total | 376.5 | 3,369 | 2,803 | |

REFINEMENT

| | |
|---|---|
| R_{cryst} (%) | 28.1 |
| R_{free} (%) | 35.9 |

¹Rmerge = Σ|Ij − <I>|/ΣIj
²Also included in the model was one Mg²⁺ and one Zn²⁺ ion [Zhang et al., Cell 98: 811-824 (1999); U.S. Ser. No. 09/396,651, Filed Sep. 15, 1999] and one Rif molecule [Brufani et al., J. Molec.Biol. 87: 409-435 (1974)].

Overall Structure: Consistent with the fact that all mapped Rif^{R} mutants occur in rpoB (FIG. 1), rifampicin makes contacts only with the RNAP β subunit in a close complementary fit to its binding pocket deep within the main DNA/RNA channel. Clearly, rifampicin does not bind directly at the RNAP active site (FIG. 3b). The closest approach of rifampicin to the active site, defined as the distance between the active site Mg²⁺ and C38 of rifampicin (see FIG. 3c), is 12.1 Å.

Detailed Interactions: A large number of rifampicin derivatives have been investigated for antimicrobial activity. In general, modification of the ansa bridge, or modifications that alter the conformation of the ansa bridge, reduce activity. Other structural features of the antibiotic that are particularly critical for activity include the napthol ring with oxygen atoms (O1 and O2) at C1 and C8, and unsubstituted hydroxyls (O10 and O9) at C21 and C23 (see FIG. 3c) [Arora, Acta Crystall. B37:152-157 (1981); Arora, Molecular Pharmacology 23:133-140 (1983); Arora, J.Med.Chem. 28:1099-1102 (1985); Arora and Main, J. Antibiot. 37:178-181 (1984); Brufani et al., J. Molec.Biol. 87:409-435 (1974); Lancini and Zanichelli, In Structure-activity Relationship in Semisynthetic Antibiotics, D. Perlaman, ed. (Academic Press), pp. 531-600 (1977); Sensi et al., Rev.Infect.Dis., 5 Supp.3:402-406 (1983)]. Most rifampicin modifications that retain activity involve substitutions at C3 of the napthol ring, which have only modulatory effects on in vitro activity.

These results can be explained by the structural details of the Rif-RNAP complex (FIGS. 4a-4b and 5a-5b). A cluster of hydrophobic residues (L391, L413, G414, 1452) line one wall of the Rif binding pocket and make van-der-Waals contact with the napthol ring and the methyl group at C7. One end of the binding pocket (the bottom in FIGS. 4a-4b) is formed by Q390. The alkyl chain of Q390 makes van-der-Waals contact with Rif C28 and C29, while the polar head group may interact with O5. Protein groups are positioned to make hydrogen bonds with each of the four critical hydroxyls of rifampicin: R409 with O1, Q393 and S411 with O2, and D396 and H406 with O10. O9 and O10 are also in position to interact with the backbone amide and carboxyl of F394, respectively. O8 of rifampicin is also positioned to make a potential hydrogen bond with the backbone amide of F394.

D396 contributes to the binding interface in several ways. In addition to forming a potential hydrogen bond with O10 of rifampicin, it forms the top end of the binding pocket (in FIGS. 4a-4b) by making van-der-Waals contact with C18-C21, and C31. Moreover, the negative charge of D396 may be important for neutralizing the positive charges of two nearby side chains, R405 and R409 (FIGS. 4a-4b), each about 6 Å away. The charge neutralization might be important for the binding of the relatively apolar of rifampicin. Most Rif^{R} mutants at amino acid residue396 substitute a large, bulky group that would likely interfere with rifampicin binding and would not have the correct geometry for hydrogen bonding O10 (Y), or else substitute an apolar group (V, G, or A) with no hydrogen bonding ability. One of these mutants, D396V (amino acid position 516 in E. coli), was among the original, strong Rif^{R} mutants mapped by Ovchinnikov et al. [Molec.Gen.Genet.190:344-348 (1983)], pointing to the importance of this residue in forming the rifampicin binding interface. Another mutant identified in E. coli, however (D396N), is isosteric with aspartic acid and would likely maintain the hydrogen bond with O10. Nevertheless, this substitution yields weak Rif^{R} [Lisitsyn et al., Bioorg Khim 10:127-128 (1984)], which is likely caused by the loss of the negative charge at this position.

Rifampicin has a partial +-charge, localized at N4 (FIG. 3c). A negatively-charged residue, E445, is situated nearby and may contribute to the rifampicin binding site by neutralizing this charge. This is not likely to be a strong effect, as many rifampicin derivatives with equal or stronger activity than rifampicin do not have this partial charge. E445 is the only residue close enough to rifampicin to be involved in potentially direct interactions (FIGS. 4a-4b) for which a Rif^{R} mutant has not been reported. However, this residue is universally conserved as either glutamic acid or aspartic acid in a segment of β region D that is invariantly present in prokaryotes, chloroplast, archaebacteria, and eukaryotes [Allison et al., Cell 42:599-610 (1985); Sweetser et al., Proc.Natl.Acad.Sci. USA 84:1192-1196 (1987)], pointing to its importance for the basic function of RNAP.

Thus, of the 12 residues that are close enough to rifampicin to make direct interactions (including backbone interactions with F394; FIGS. 4a-4b), 11 mutate to a Rif^{R} phenotype. The twelfth position, E445, is highly conserved so that its substitution would likely be lethal and consequently not be detectable as a Rif^{R} mutation.

Twelve additional positions have been identified at which substitution gives rise to Rif^{R} (FIG. 1). These residues surround the Rif binding pocket but do not make direct interactions with the antibiotic (FIGS. 5a-5b). In every case, the Rif^{R} mutations involve replacement by a different sized amino acid side-chain (almost always substituting a small residue with a more bulky one), or else involve adding or removing a proline residue. These substitutions would likely affect the folding or packing of the protein in the local vicinity of the substituted residue, causing distortions of the Rif binding pocket.

Mechanism of RNAP Inhibition by Rif: The effects of rifampicin on RNAP in each stage of the transcription cycle have been probed using detailed kinetic analyses. Rifampicin has essentially no effect on specific promoter binding and open complex formation [Hinkle et al., J.Molec.Biol.,70, 209-220 (1972); McClure and Cech, J.Biol.Chem. 253:8949-8956 (1978)]. A small increase (about 2-fold) in the apparent Km for initiating substrate binding in the enzyme's i-site (the 5′-nucleotide) was observed, but the binding of the incoming nucleotide substrate in the i+1 site (the 3′-nucleotide), and the formation of the first phosphodiester bond were largely unaffected [McClure and Cech, J.Biol.Chem. 253:8949-8956 (1978)]. The dominant effect of rifampicin binding on RNAP activity was a total blockage of synthesis of the second (when transcription was initiated with a nucleoside triphosphate) or third (when transcription was initiated with a nucleoside di- or monophosphate) phosphodiester bond [McClure and Cech, J.Biol.Chem. 253:8949-8956 (1978)]. Since synthesis of the first and second phosphodiester bond can occur in the presence of rifampicin, the antibiotic does not interfere with substrate binding, catalytic activity, or the intrinsic translocation mechanism of the RNAP. After RNAP has synthesized a long transcript and entered the elongation phase, it becomes totally resistant to rifampicin. These properties led to the proposal that rifampicin inhibits RNAP through a simple steric block of the path of the elongating RNA at the 5′-end [McClure and Cech, J.Biol.Chem. 253:8949-8956 (1978)]. Whether rifampicin directly blocked the path of the RNA, or if blockage was an indirect effect due to a conformational change in the RNAP induced by rifampicin binding, could not be distinguished. It has alternatively been proposed that rifampicin exerts its effect allosterically by decreasing the affinity of the RNAP for short RNA transcripts [Schulz and Zillig, Nucl.AcidsRes. 9:6889-6906 (1981)].

The Rif-RNAP crystal structure explains the results described above and strongly supports the simple steric block mechanism, see, atomic coordinates included in Table 2 [McClure and Cech, J.Biol.Chem. 253:8949-8956 (1978)]. Rifampicin directly abuts the base of a loop that comprises the C-terminal part of the β conserved region D (amino acid residues 443-451, shaded red in FIGS. 5a-5b), and a cluster of Rif^{R} mutants, Rif cluster I (FIG. 1), flanks this region. Modeling suggests that this loop, which contains several nearly universally conserved residues, participates in forming the binding site for the base-pair at +1 in the transcription complex [Korzheva et al., Science 289:619-625 (2000)], so effects of rifampicin on the Km for the initiating substrate are not surprising. However, rifampicin does not directly contact the end of this loop. In addition, conformational changes of the protein in this region are not indicated from the structural data, consistent with the observation that the effect of rifampicin on this region is small.

The principal effect of rifampicin is seen in the context of a model of the transcriptionally active ternary complex [Korzheva et al., Science 289:619-625 (2000)] containing RNAP, DNA template, and RNA transcript (FIG. 6a). In FIG. 6, only the RNAP active site Mg²⁺ and the 9-basepair RNA/DNA hybrid (from +1 to −7) from the ternary complex model are shown. The rest of the RNAP and nucleic acids are omitted for clarity. Also shown is the atomic model of rifampicin as it would be positioned in its binding site on the β subunit.

It can be seen that the two substrate nucleotides, at +1 (green) and −1, are not directly affected by the presence of rifampicin so that RNAP can bind and catalyze the formation of a phosphodiester bond between the two substrates in the presence of the antibiotic. With a transcript length of 3 nucleotides (nt), however, the 5′-phosphates of the 5′-nucleotide (at −2) sterically clash with rifampicin, and the nucleotides further upstream (−3 to −5) severely clash with rifampicin. At the same time, rifampicin does not interfere with the DNA (grey). Thus, the structure, in combination with the ternary complex model, explains the biochemical data on the mechanism of rifampicin inhibition, provides strong support for the proposal that rifampicin sterically blocks the path of the elongating RNA transcript at the 5′-end, and indicates that the blockage is a direct consequence of rifampicin binding in its site. The model further suggests why transcripts initiated with nucleoside triphosphates are blocked after the first phosphodiester bond, while transcripts initiated with nucleoside di- or monophosphates are blocked after the second phosphodiester bond. In the model, the nucleoside monophosphate in the transcript at the −2 position clashes only slightly with rifampicin, while the presence of a 5′-triphosphate at the −2 position would extend into rifampicin.

Core RNAP can bind a pre-formed minimal nucleic acid scaffold of RNA/DNA oligonucleotides (FIG. 6b, top) to yield functional ternary elongation complexes [Korzheva et al., Science 289:619-625 (2000)]. Order of addition experiments were performed using this system in order to assess whether rifampicin and RNA binding were competetive (FIG. 6b). The DNA component of the scaffold was annealed with varying lengths of RNA transcript, and the effect of rifampicin on the sequence-dependent extension of RNA by one nucleotide (radioactively-labeled CTP) added before or after the oligonucleotides was assayed at room temperature. In the case of E. coli core RNAP in the absence of rifampicin the RNA transcript was extended with nearly equal efficiency regardless of its length within a range of 3-7 nucleotides (FIG. 6b, lanes 11-15). When rifampicin was added prior to the nucleotide scaffold, the RNAP was unable to extend any of the RNA oligos, regardless of length (lanes 1-5), indicating that rifampicin occupied its site and blocked the extension and/or binding of all of the transcripts. When the scaffold was added prior to rifampicin addition, rifampicin was able to occupy its site and block the extension of the 3-nucleotide transcript (lane 6), but had no effect on the extension of the longer transcripts (lanes 7-10), presumably because rifampicin could not access its binding site due to the presence of the longer RNA transcripts (FIG. 6a). This result is consistent with the early data that rifampicin inhibits the RNA extension from 2 to 3 nucleotides if the 5′-nucleoside is tri-phosphorylated, but inhibits extension from 3 to 4 nucleotides if the 5′-nucleoside is mono- or di-phosphorylated [McClure and Cech, J.Biol.Chem. 253:8949-8956 (1978)] since the synthetic RNA oligos lack 5′-phosphates.

Similar experiments were performed with Taq core RNAP (FIG. 6b, lanes 16-30). In the absence of rifampicin, the efficiency of transcript extension was strongly dependent on the transcript length (lanes 26-30). Extension of the shortest transcripts was barely detectable, suggesting that, unlike E. coli RNAP, Taq, core RNAP does not bind and stabilize the short, intrinsically unstable RNA/DNA hybrids. In the presence of rifampicin, a generalized inhibition of transcript extension was observed regardless of the order of addition or of the transcript length (lanes 16-25). These results can be explained by the low binding affinity of Taq core RNAP for both rifampicin and for short RNA transcripts compared with E. coli core RNAP. The low affinities imply fast off-rates, which would allow equilibrium to be established between the rifampicin and scaffold binding during the time of the assay.

### Discussion

The 3.3 Å X-ray crystal structure of Taq core RNAP complexed with rifampicin is disclosed herein. Though Taq RNAP is less sensitive to rifampicin than E.coli rifampicin, at sufficiently high concentrations the antibiotic binds and inhibits the enzyme. Significantly, however, the inhibition of Taq RNAP by rifampicin occurs through the same biochemical mechanism as E. coli RNAP, and the disposition of the Rif-site with respect to the active site is identical to E. coli RNAP as well as with other prokaryotic RNAPs (FIGS. 2a-2d). Therefore, the structural information provided herein is relevant for all bacteria RNAPs.

The relative insensitivity of Taq RNAP to rifampicin is likely due to amino acid substitutions in Taq RNAP compared with other, more Rif-sensitive RNAPs. The 12 residues close enough to interact directly with the rifampicin are identical between E. coli, Taq, and M. tuberculosis (marked yellow in FIG. 1). Among the 11 secondary positions that do not directly interact with rifampicin but likely affect rifampicin binding indirectly, 5 are substituted in Taq RNAP (amino acid residues 387, 395, 398, 453, and 566; FIG. 1). Three of these positions, 387, 398, and 453, contain amino acids that are not dramatically different in overall size from their E. coli and M. tuberculosis counterparts and one would predict that these residues are not the origin of the Taq RNAP insensitivity to rifampicin. Position 566 is highly conserved among all RNAPs as either a lysine or an arginine (the homologous position is an arginine in both E. coli and M. tuberculosis) but is a threonine in Taq RNAP. This substitution is unlikely to be the main determinant of the Taq RNAP Rif insensitivity, however, since mutating Taq Thr566 to an arginine has little effect on the Rif^{R} of the enzyme when assayed at 45° C. This leaves position 395, which is highly conserved as a hydrophobic residue among all RNAPs. In E. coli and M. tuberculosis this position is a methionine, but in Taq it is a lysine. Taq Lys395 appears to participate in buried salt-bridges with Asp124 and Asp133 that may contribute to the thermostability of the protein. This non-conservative substitution (lysine for methionine) could affect the local path of the polypeptide backbone, and is immediately adjacent to Phe394, the backbone amide and carboxyl of which appear to be involved in important interactions with the rifampicin (FIGS. 4a-4b).

All but one of the residues that are close enough to rifampicin to participate in direct interactions are known to mutate to strong Rif^{R} (FIGS. 4a-4b). However, additional residues could be important for the formation of the Rif binding pocket but not revealed as Rif^{R} mutants if they are necessary for basic RNAP function. As mentioned above, the four regions of the β subunit that harbor Rif^{R} mutants are highly conserved among prokaryotes (FIG. 1), but the much weaker homology with archaebacterial and eukaryotic RNAPs, combined with the fact that so many Rif^{R} mutations have been discovered, indicate that these regions are not critical to RNAP function in vivo. Nevertheless, some Rif^{R} mutations do have profound functional effects [Jin and Gross, J.Biol.Chem. 266:14478-14485 (1991); Landick et al., Genes Develop. 4:1623-1636 (1990)], and E. coli strains with Rif^{R} RNAP have been shown to be at a competetive disadvantage to wild type E. coli in the absence of rifampicin [Jin and Gross, J.Bact.171:5229-5231 (1989)].

The clinical success of rifampicin proves that the bacterial RNAP is an excellent target for antimicrobials. The structure and available genetic and biochemical data suggest that the design of modified versions of rifampicin to overcome the effects of Rif^{R} mutations may lead to incremental improvements, though may not lead to a “wonder” drug because of the apparently small functional penalties of mutating this region of the RNAP, and the variety of amino acid positions and mutations that result in Rif^{R} (FIG. 1). In contrast, however, the findings from clinical isolates of Rif^{R} M. tuberculosis are rather encouraging. Thus, although the Rif^{R} mutations are spread over 15 positions of rpoB, 77% of all the mutations isolated involved substitutions at one of only two positions, corresponding to Taq amino acid residues 406 and 411. If a third amino acid residue is included, i.e., (Taq 396) a combined 86% of all the reported mutants are accounted for.

One important conclusion from the present disclosure emerges regarding the inhibitory mechanism of rifampicin, i.e., it is a simple steric block of transcription elongation. Thus, the powerful effects of rifampicin do not stem from the details of its chemical structure, and do not involve interference with the catalytic activity of the RNAP, e.g., by mimicking substrates or a transition state of the polymerization reaction. Indeed, such an inhibitor would likely act on features that are highly conserved between prokaryotes and eukaryotes, rendering the inhibitor useless as an antimicrobial agent. Rather, the effects of rifampicin depend only on its ability to bind tightly to a relatively non-conserved part of the structure, disrupting a critical RNAP function by virtue of its presence. Decades of functional studies [Chamberlin, Harvey Lectures 88:1-21 (1993); Korzheva et al.,Cold Spring Harbor Symposia on Quantitative Biology 63:337-345 (1998); Mustaev et al., Proc.Nat.Acad.Sci. USA 91:12036-12040 (1994); and Nudler, J.Molec.Biol. 288:1-12 (1999)], and more recent structural evidence [Cramer et al., Science 288:640-649 (2000); Korzheva et al., Science 289:619-625 (2000); Mooney and Landick, Cell 98:687-690(1999); Zhang et al., Cell 98:811-824 (1999); U.S. Ser. No. 09/396,651, Filed Sep. 15, 1999, the contents of which are hereby incorporated by reference in their entireties] indicate that cellular RNAPs operate as complex molecular machines, with extensive interactions with the template DNA, product RNA [Korzheva et al., Science 289:619-625 (2000)], and other regulatory molecules. Thus, many additional distinct sites exist where the tight binding of a small molecule (i.e., a novel antibiotic) would disrupt critical features of the functional mechanism of bacterial RNAPs. Such distinct sites can be readily identified through the structural information provided by the present invention.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

It is further to be understood that all base sizes or amino acid sizes, and all molecular weight or molecular mass values, given for nucleic acids or polypeptides are approximate, and are provided for description.

Various publications are cited herein, the disclosures of which are incorporated by reference in their entireties.

TABLE 2

| REMARK | Written by O version 6.2.1 |
|---|---|
| REMARK | Thu Oct 19 16:44:44 2000 |
| CRYST1 | 199.450 199.450 289.130 90.00 90.00 90.00 |

| | | | | |
|---|---|---|---|---|
| ORIGX1 | 1.000000 | 0.000000 | 0.000000 | 0.00000 |
| ORIGX2 | 0.000000 | 1.000000 | 0.000000 | 0.00000 |
| ORIGX3 | 0.000000 | 0.000000 | 1.000000 | 0.00000 |
| SCALE1 | 0.005014 | 0.000000 | 0.000000 | 0.00000 |
| SCALE2 | 0.000000 | 0.005014 | 0.000000 | 0.00000 |
| SCALE3 | 0.000000 | 0.000000 | 0.003459 | 0.00000 |

| | | | | | | | | | | | |
|---|---|---|---|---|---|---|---|---|---|---|---|
| ATOM | 1 | CB | ALA | A | 6 | −0.674 | 65.559 | 73.225 | 1.00 | 184.01 | 6 |
| ATOM | 1 | CB | ALA | A | 6 | −0.674 | 65.559 | 73.225 | 1.00 | 184.01 | 6 |
| ATOM | 2 | C | ALA | A | 6 | −1.081 | 63.492 | 71.882 | 1.00 | 150.25 | 6 |
| ATOM | 3 | O | ALA | A | 6 | −1.961 | 62.636 | 71.787 | 1.00 | 150.75 | 8 |
| ATOM | 4 | N | ALA | A | 6 | −2.906 | 65.120 | 72.256 | 1.00 | 150.94 | 7 |
| ATOM | 5 | CA | ALA | A | 6 | −1.440 | 64.960 | 72.050 | 1.00 | 150.69 | 6 |
| ATOM | 6 | N | LYS | A | 7 | 0.220 | 63.209 | 71.866 | 1.00 | 121.92 | 7 |
| ATOM | 7 | CA | LYS | A | 7 | 0.729 | 61.855 | 71.667 | 1.00 | 120.02 | 6 |
| ATOM | 8 | CB | LYS | A | 7 | −0.036 | 60.821 | 72.496 | 1.00 | 156.94 | 6 |
| ATOM | 9 | CG | LYS | A | 7 | 0.137 | 60.890 | 73.994 | 1.00 | 157.98 | 6 |
| ATOM | 10 | CD | LYS | A | 7 | −0.665 | 59.764 | 74.624 | 1.00 | 159.08 | 6 |
| ATOM | 11 | CE | LYS | A | 7 | −0.540 | 59.750 | 76.131 | 1.00 | 160.31 | 6 |
| ATOM | 12 | NZ | LYS | A | 7 | −1.332 | 58.638 | 76.731 | 1.00 | 161.74 | 7 |
| ATOM | 13 | C | LYS | A | 7 | 0.489 | 61.553 | 70.201 | 1.00 | 118.48 | 6 |
| ATOM | 14 | O | LYS | A | 7 | 1.116 | 60.665 | 69.623 | 1.00 | 118.87 | 8 |
| ATOM | 15 | N | ALA | A | 8 | −0.435 | 62.308 | 69.613 | 1.00 | 91.47 | 7 |
| ATOM | 16 | CA | ALA | A | 8 | −0.801 | 62.143 | 68.218 | 1.00 | 88.83 | 6 |
| ATOM | 17 | CB | ALA | A | 8 | −2.077 | 61.305 | 68.118 | 1.00 | 71.07 | 6 |
| ATOM | 18 | C | ALA | A | 8 | −1.005 | 63.487 | 67.524 | 1.00 | 86.40 | 6 |
| ATOM | 19 | O | ALA | A | 8 | −1.539 | 64.428 | 68.112 | 1.00 | 86.27 | 8 |
| ATOM | 20 | N | PRO | A | 9 | −0.549 | 63.597 | 66.267 | 1.00 | 53.91 | 7 |
| ATOM | 21 | CD | PRO | A | 9 | 0.517 | 62.722 | 65.741 | 1.00 | 69.31 | 6 |
| ATOM | 22 | CA | PRO | A | 9 | −0.669 | 64.809 | 65.449 | 1.00 | 52.02 | 6 |
| ATOM | 23 | CB | PRO | A | 9 | 0.674 | 64.864 | 64.756 | 1.00 | 68.44 | 6 |
| ATOM | 24 | CG | PRO | A | 9 | 0.906 | 63.412 | 64.453 | 1.00 | 69.17 | 6 |
| ATOM | 25 | C | PRO | A | 9 | −1.824 | 64.699 | 64.450 | 1.00 | 49.71 | 6 |
| ATOM | 26 | O | PRO | A | 9 | −2.494 | 63.679 | 64.378 | 1.00 | 48.85 | 8 |
| ATOM | 27 | N | VAL | A | 10 | −2.040 | 65.746 | 63.670 | 1.00 | 56.71 | 7 |
| ATOM | 28 | CA | VAL | A | 10 | −3.117 | 65.753 | 62.692 | 1.00 | 55.46 | 6 |
| ATOM | 29 | CB | VAL | A | 10 | −4.120 | 66.872 | 63.012 | 1.00 | 55.12 | 6 |
| ATOM | 30 | CG1 | VAL | A | 10 | −5.149 | 67.005 | 61.904 | 1.00 | 55.17 | 6 |
| ATOM | 31 | CG2 | VAL | A | 10 | −4.795 | 66.581 | 64.335 | 1.00 | 54.29 | 6 |
| ATOM | 32 | C | VAL | A | 10 | −2.554 | 65.958 | 61.296 | 1.00 | 55.42 | 6 |
| ATOM | 33 | O | VAL | A | 10 | −2.573 | 67.064 | 60.756 | 1.00 | 55.98 | 8 |
| ATOM | 34 | N | PHE | A | 11 | −2.069 | 64.871 | 60.711 | 1.00 | 63.05 | 7 |
| ATOM | 35 | CA | PHE | A | 11 | −1.454 | 64.903 | 59.392 | 1.00 | 62.54 | 6 |
| ATOM | 36 | CB | PHE | A | 11 | −0.919 | 63.509 | 59.053 | 1.00 | 119.96 | 6 |
| ATOM | 37 | CG | PHE | A | 11 | −0.075 | 63.460 | 57.809 | 1.00 | 120.51 | 6 |
| ATOM | 38 | CD1 | PHE | A | 11 | 0.806 | 64.494 | 57.500 | 1.00 | 120.57 | 6 |
| ATOM | 39 | CD2 | PHE | A | 11 | −0.135 | 62.356 | 56.961 | 1.00 | 121.37 | 6 |
| ATOM | 40 | CE1 | PHE | A | 11 | 1.616 | 64.428 | 56.366 | 1.00 | 119.73 | 6 |
| ATOM | 41 | CE2 | PHE | A | 11 | 0.669 | 62.280 | 55.827 | 1.00 | 120.93 | 6 |
| ATOM | 42 | CZ | PHE | A | 11 | 1.547 | 63.318 | 55.527 | 1.00 | 120.06 | 6 |
| ATOM | 43 | C | PHE | A | 11 | −2.340 | 65.419 | 58.266 | 1.00 | 62.10 | 6 |
| ATOM | 44 | O | PHE | A | 11 | −2.671 | 64.685 | 57.338 | 1.00 | 61.58 | 8 |
| ATOM | 45 | N | THR | A | 12 | −2.721 | 66.690 | 58.355 | 1.00 | 47.43 | 7 |
| ATOM | 46 | CA | THR | A | 12 | −3.533 | 67.338 | 57.328 | 1.00 | 47.79 | 6 |
| ATOM | 47 | CB | THR | A | 12 | −3.713 | 68.834 | 57.647 | 1.00 | 108.24 | 6 |
| ATOM | 48 | OG1 | THR | A | 12 | −4.382 | 68.979 | 58.909 | 1.00 | 108.66 | 8 |
| ATOM | 49 | CG2 | THR | A | 12 | −4.523 | 69.512 | 56.564 | 1.00 | 109.46 | 6 |
| ATOM | 50 | C | THR | A | 12 | −2.752 | 67.169 | 56.021 | 1.00 | 47.46 | 6 |
| ATOM | 51 | O | THR | A | 12 | −1.552 | 66.897 | 56.058 | 1.00 | 47.91 | 8 |
| ATOM | 52 | N | ALA | A | 13 | −3.396 | 67.330 | 54.867 | 1.00 | 38.72 | 7 |
| ATOM | 53 | CA | ALA | A | 13 | −2.661 | 67.120 | 53.621 | 1.00 | 38.79 | 6 |
| ATOM | 54 | CB | ALA | A | 13 | −2.509 | 65.590 | 53.374 | 1.00 | 19.61 | 6 |
| ATOM | 55 | C | ALA | A | 13 | −3.163 | 67.803 | 52.350 | 1.00 | 38.10 | 6 |
| ATOM | 56 | O | ALA | A | 13 | −3.149 | 67.194 | 51.295 | 1.00 | 37.13 | 8 |
| ATOM | 57 | N | THR | A | 14 | −3.574 | 69.064 | 52.441 | 1.00 | 50.93 | 7 |
| ATOM | 58 | CA | THR | A | 14 | −4.061 | 69.814 | 51.268 | 1.00 | 51.96 | 6 |
| ATOM | 59 | CB | THR | A | 14 | −3.993 | 71.381 | 51.485 | 1.00 | 77.70 | 6 |
| ATOM | 60 | OG1 | THR | A | 14 | −4.992 | 71.801 | 52.429 | 1.00 | 76.23 | 8 |
| ATOM | 61 | CG2 | THR | A | 14 | −4.213 | 72.125 | 50.161 | 1.00 | 77.77 | 6 |
| ATOM | 62 | C | THR | A | 14 | −3.253 | 69.477 | 50.007 | 1.00 | 52.16 | 6 |
| ATOM | 63 | O | THR | A | 14 | −2.238 | 70.113 | 49.713 | 1.00 | 51.78 | 8 |
| ATOM | 64 | N | THR | A | 15 | −3.722 | 68.476 | 49.264 | 1.00 | 38.17 | 7 |
| ATOM | 65 | CA | THR | A | 15 | −3.062 | 68.038 | 48.042 | 1.00 | 39.18 | 6 |
| ATOM | 66 | CB | THR | A | 15 | −3.497 | 66.590 | 47.671 | 1.00 | 21.08 | 6 |
| ATOM | 67 | OG1 | THR | A | 15 | −3.070 | 66.293 | 46.345 | 1.00 | 20.30 | 8 |
| ATOM | 68 | CG2 | THR | A | 15 | −4.988 | 66.426 | 47.736 | 1.00 | 20.79 | 6 |
| ATOM | 69 | C | THR | A | 15 | −3.301 | 68.987 | 46.863 | 1.00 | 39.71 | 6 |
| ATOM | 70 | O | THR | A | 15 | −3.672 | 70.150 | 47.049 | 1.00 | 38.75 | 8 |
| ATOM | 71 | N | GLN | A | 16 | −3.060 | 68.477 | 45.659 | 1.00 | 87.11 | 7 |
| ATOM | 72 | CA | GLN | A | 16 | −3.224 | 69.223 | 44.415 | 1.00 | 90.16 | 6 |
| ATOM | 73 | CB | GLN | A | 16 | −2.064 | 70.199 | 44.226 | 1.00 | 98.47 | 6 |
| ATOM | 74 | CG | GLN | A | 16 | −2.428 | 71.637 | 44.527 | 1.00 | 98.74 | 6 |
| ATOM | 75 | CD | GLN | A | 16 | −3.310 | 72.240 | 43.459 | 1.00 | 98.04 | 6 |
| ATOM | 76 | OE1 | GLN | A | 16 | −2.845 | 72.994 | 42.603 | 1.00 | 97.07 | 8 |
| ATOM | 77 | NE2 | GLN | A | 16 | −4.591 | 71.900 | 43.494 | 1.00 | 98.68 | 7 |
| ATOM | 78 | C | GLN | A | 16 | −3.274 | 68.263 | 43.229 | 1.00 | 91.21 | 6 |
| ATOM | 79 | O | GLN | A | 16 | −3.465 | 67.059 | 43.400 | 1.00 | 91.96 | 8 |
| ATOM | 80 | N | GLY | A | 17 | −3.103 | 68.801 | 42.028 | 1.00 | 109.33 | 7 |
| ATOM | 81 | CA | GLY | A | 17 | −3.130 | 67.966 | 40.843 | 1.00 | 110.69 | 6 |
| ATOM | 82 | C | GLY | A | 17 | −1.741 | 67.744 | 40.276 | 1.00 | 110.89 | 6 |
| ATOM | 83 | O | GLY | A | 17 | −1.408 | 68.262 | 39.208 | 1.00 | 111.99 | 8 |
| ATOM | 84 | N | ASP | A | 18 | −0.930 | 66.964 | 40.982 | 1.00 | 49.15 | 7 |
| ATOM | 85 | CA | ASP | A | 18 | 0.429 | 66.696 | 40.533 | 1.00 | 48.27 | 6 |
| ATOM | 86 | CB | ASP | A | 18 | 0.425 | 66.081 | 39.121 | 1.00 | 95.31 | 6 |
| ATOM | 87 | CG | ASP | A | 18 | 0.285 | 64.555 | 39.130 | 1.00 | 95.93 | 6 |
| ATOM | 88 | OD1 | ASP | A | 18 | 1.109 | 63.883 | 39.785 | 1.00 | 96.31 | 8 |
| ATOM | 89 | OD2 | ASP | A | 18 | −0.639 | 64.026 | 38.474 | 1.00 | 95.54 | 8 |
| ATOM | 90 | C | ASP | A | 18 | 1.228 | 68.004 | 40.539 | 1.00 | 46.85 | 6 |
| ATOM | 91 | O | ASP | A | 18 | 1.470 | 68.615 | 39.496 | 1.00 | 46.35 | 8 |
| ATOM | 92 | N | HIS | A | 19 | 1.604 | 68.418 | 41.747 | 1.00 | 95.41 | 7 |
| ATOM | 93 | CA | HIS | A | 19 | 2.388 | 69.623 | 42.064 | 1.00 | 93.95 | 6 |
| ATOM | 94 | CB | HIS | A | 19 | 2.406 | 70.663 | 40.916 | 1.00 | 119.86 | 6 |
| ATOM | 95 | CG | HIS | A | 19 | 1.071 | 71.252 | 40.570 | 1.00 | 121.65 | 6 |
| ATOM | 96 | CD2 | HIS | A | 19 | 0.384 | 71.270 | 39.402 | 1.00 | 121.92 | 6 |
| ATOM | 97 | ND1 | HIS | A | 19 | 0.319 | 71.982 | 41.465 | 1.00 | 121.96 | 7 |
| ATOM | 98 | CE1 | HIS | A | 19 | −0.772 | 72.424 | 40.864 | 1.00 | 122.23 | 6 |
| ATOM | 99 | NE2 | HIS | A | 19 | −0.756 | 72.005 | 39.611 | 1.00 | 122.38 | 7 |
| ATOM | 100 | C | HIS | A | 19 | 1.835 | 70.218 | 43.363 | 1.00 | 91.34 | 6 |
| ATOM | 101 | O | HIS | A | 19 | 0.882 | 69.678 | 43.940 | 1.00 | 90.88 | 8 |
| ATOM | 102 | N | TYR | A | 20 | 2.423 | 71.316 | 43.833 | 1.00 | 59.30 | 7 |
| ATOM | 103 | CA | TYR | A | 20 | 2.001 | 71.911 | 45.101 | 1.00 | 56.72 | 6 |
| ATOM | 104 | CB | TYR | A | 20 | 0.675 | 72.641 | 44.992 | 1.00 | 84.02 | 6 |
| ATOM | 105 | CG | TYR | A | 20 | 0.845 | 74.124 | 44.855 | 1.00 | 86.19 | 6 |
| ATOM | 106 | CD1 | TYR | A | 20 | 0.937 | 74.719 | 43.593 | 1.00 | 86.75 | 6 |
| ATOM | 107 | CE1 | TYR | A | 20 | 1.114 | 76.095 | 43.451 | 1.00 | 87.24 | 6 |
| ATOM | 108 | CD2 | TYR | A | 20 | 0.936 | 74.940 | 45.982 | 1.00 | 87.02 | 6 |
| ATOM | 109 | CE2 | TYR | A | 20 | 1.115 | 76.321 | 45.852 | 1.00 | 87.64 | 6 |
| ATOM | 110 | CZ | TYR | A | 20 | 1.203 | 76.889 | 44.581 | 1.00 | 87.77 | 6 |
| ATOM | 111 | OH | TYR | A | 20 | 1.389 | 78.244 | 44.436 | 1.00 | 89.34 | 8 |
| ATOM | 112 | C | TYR | A | 20 | 1.847 | 70.778 | 46.072 | 1.00 | 53.80 | 6 |
| ATOM | 113 | O | TYR | A | 20 | 2.391 | 69.707 | 45.867 | 1.00 | 54.73 | 8 |
| ATOM | 114 | N | GLY | A | 21 | 1.100 | 70.989 | 47.132 | 1.00 | 50.16 | 7 |
| ATOM | 115 | CA | GLY | A | 21 | 0.950 | 69.902 | 48.069 | 1.00 | 47.19 | 6 |
| ATOM | 116 | C | GLY | A | 21 | 1.471 | 70.248 | 49.442 | 1.00 | 44.51 | 6 |
| ATOM | 117 | O | GLY | A | 21 | 2.361 | 69.590 | 49.973 | 1.00 | 44.32 | 8 |
| ATOM | 118 | N | GLU | A | 22 | 0.924 | 71.314 | 50.005 | 1.00 | 39.88 | 7 |
| ATOM | 119 | CA | GLU | A | 22 | 1.293 | 71.724 | 51.335 | 1.00 | 37.92 | 6 |
| ATOM | 120 | CB | GLU | A | 22 | 0.488 | 72.965 | 51.716 | 1.00 | 62.99 | 6 |
| ATOM | 121 | CG | GLU | A | 22 | 0.217 | 73.904 | 50.537 | 1.00 | 64.94 | 6 |
| ATOM | 122 | CD | GLU | A | 22 | −0.739 | 75.046 | 50.885 | 1.00 | 67.11 | 6 |
| ATOM | 123 | OE1 | GLU | A | 22 | −1.688 | 74.806 | 51.671 | 1.00 | 67.85 | 8 |
| ATOM | 124 | OE2 | GLU | A | 22 | −0.555 | 76.174 | 50.358 | 1.00 | 67.53 | 8 |
| ATOM | 125 | C | GLU | A | 22 | 0.843 | 70.511 | 52.145 | 1.00 | 35.14 | 6 |
| ATOM | 126 | O | GLU | A | 22 | −0.221 | 69.953 | 51.886 | 1.00 | 35.06 | 8 |
| ATOM | 127 | N | PHE | A | 23 | 1.648 | 70.072 | 53.098 | 1.00 | 45.71 | 7 |
| ATOM | 128 | CA | PHE | A | 23 | 1.262 | 68.914 | 53.889 | 1.00 | 44.96 | 6 |
| ATOM | 129 | CB | PHE | A | 23 | 2.104 | 67.698 | 53.494 | 1.00 | 29.76 | 6 |
| ATOM | 130 | CG | PHE | A | 23 | 2.074 | 67.389 | 52.023 | 1.00 | 26.17 | 6 |
| ATOM | 131 | CD1 | PHE | A | 23 | 0.873 | 67.327 | 51.337 | 1.00 | 26.14 | 6 |
| ATOM | 132 | CD2 | PHE | A | 23 | 3.244 | 67.140 | 51.327 | 1.00 | 24.41 | 6 |
| ATOM | 133 | CE1 | PHE | A | 23 | 0.844 | 67.019 | 49.981 | 1.00 | 25.18 | 6 |
| ATOM | 134 | CE2 | PHE | A | 23 | 3.217 | 66.832 | 49.973 | 1.00 | 23.87 | 6 |
| ATOM | 135 | CZ | PHE | A | 23 | 2.020 | 66.773 | 49.304 | 1.00 | 24.25 | 6 |
| ATOM | 136 | C | PHE | A | 23 | 1.451 | 69.216 | 55.364 | 1.00 | 45.13 | 6 |
| ATOM | 137 | O | PHE | A | 23 | 2.564 | 69.127 | 55.880 | 1.00 | 46.47 | 8 |
| ATOM | 138 | N | VAL | A | 24 | 0.359 | 69.559 | 56.044 | 1.00 | 29.36 | 7 |
| ATOM | 139 | CA | VAL | A | 24 | 0.421 | 69.908 | 57.457 | 1.00 | 28.70 | 6 |
| ATOM | 140 | CB | VAL | A | 24 | −0.710 | 70.840 | 57.891 | 1.00 | 26.37 | 6 |
| ATOM | 141 | CG1 | VAL | A | 24 | −0.537 | 71.165 | 59.348 | 1.00 | 27.05 | 6 |
| ATOM | 142 | CG2 | VAL | A | 24 | −0.716 | 72.115 | 57.074 | 1.00 | 26.62 | 6 |
| ATOM | 143 | C | VAL | A | 24 | 0.351 | 68.751 | 58.414 | 1.00 | 28.27 | 6 |
| ATOM | 144 | O | VAL | A | 24 | −0.470 | 67.858 | 58.287 | 1.00 | 28.42 | 8 |
| ATOM | 145 | N | LEU | A | 25 | 1.236 | 68.776 | 59.390 | 1.00 | 24.98 | 7 |
| ATOM | 146 | CA | LEU | A | 25 | 1.223 | 67.767 | 60.413 | 1.00 | 25.10 | 6 |
| ATOM | 147 | CB | LEU | A | 25 | 2.600 | 67.635 | 61.051 | 1.00 | 20.57 | 6 |
| ATOM | 148 | CG | LEU | A | 25 | 2.998 | 66.180 | 61.263 | 1.00 | 20.16 | 6 |
| ATOM | 149 | CD1 | LEU | A | 25 | 3.158 | 65.901 | 62.743 | 1.00 | 18.63 | 6 |
| ATOM | 150 | CD2 | LEU | A | 25 | 1.941 | 65.264 | 60.635 | 1.00 | 19.90 | 6 |
| ATOM | 151 | C | LEU | A | 25 | 0.266 | 68.510 | 61.315 | 1.00 | 26.87 | 6 |
| ATOM | 152 | O | LEU | A | 25 | −0.833 | 68.853 | 60.865 | 1.00 | 26.44 | 8 |
| ATOM | 153 | N | GLU | A | 26 | 0.669 | 68.787 | 62.555 | 1.00 | 116.39 | 7 |
| ATOM | 154 | CA | GLU | A | 26 | −0.183 | 69.547 | 63.467 | 1.00 | 118.12 | 6 |
| ATOM | 155 | CB | GLU | A | 26 | −1.494 | 68.801 | 63.727 | 1.00 | 55.30 | 6 |
| ATOM | 156 | CG | GLU | A | 26 | −2.743 | 69.596 | 63.350 | 1.00 | 53.07 | 6 |
| ATOM | 157 | CD | GLU | A | 26 | −2.547 | 71.087 | 63.477 | 1.00 | 51.49 | 6 |
| ATOM | 158 | OE1 | GLU | A | 26 | −2.151 | 71.538 | 64.566 | 1.00 | 49.62 | 8 |
| ATOM | 159 | OE2 | GLU | A | 26 | −2.795 | 71.803 | 62.486 | 1.00 | 52.84 | 8 |
| ATOM | 160 | C | GLU | A | 26 | 0.466 | 69.999 | 64.786 | 1.00 | 120.58 | 6 |
| ATOM | 161 | O | GLU | A | 26 | 1.435 | 70.758 | 64.754 | 1.00 | 121.90 | 8 |
| ATOM | 162 | N | PRO | A | 27 | −0.027 | 69.541 | 65.959 | 1.00 | 79.42 | 7 |
| ATOM | 163 | CD | PRO | A | 27 | −0.943 | 68.451 | 66.354 | 1.00 | 84.24 | 6 |
| ATOM | 164 | CA | PRO | A | 27 | 0.660 | 70.056 | 67.143 | 1.00 | 79.50 | 6 |
| ATOM | 165 | CB | PRO | A | 27 | −0.342 | 69.790 | 68.241 | 1.00 | 83.86 | 6 |
| ATOM | 166 | CG | PRO | A | 27 | −0.787 | 68.400 | 67.874 | 1.00 | 84.43 | 6 |
| ATOM | 167 | C | PRO | A | 27 | 1.957 | 69.323 | 67.388 | 1.00 | 81.30 | 6 |
| ATOM | 168 | O | PRO | A | 27 | 1.970 | 68.305 | 68.076 | 1.00 | 82.18 | 8 |
| ATOM | 169 | N | LEU | A | 28 | 3.045 | 69.827 | 66.819 | 1.00 | 52.55 | 7 |
| ATOM | 170 | CA | LEU | A | 28 | 4.337 | 69.190 | 67.014 | 1.00 | 53.38 | 6 |
| ATOM | 171 | CB | LEU | A | 28 | 5.204 | 69.324 | 65.756 | 1.00 | 30.51 | 6 |
| ATOM | 172 | CG | LEU | A | 28 | 5.135 | 68.133 | 64.788 | 1.00 | 29.10 | 6 |
| ATOM | 173 | CD1 | LEU | A | 28 | 5.692 | 68.542 | 63.433 | 1.00 | 27.98 | 6 |
| ATOM | 174 | CD2 | LEU | A | 28 | 5.892 | 66.924 | 65.380 | 1.00 | 28.36 | 6 |
| ATOM | 175 | C | LEU | A | 28 | 5.014 | 69.835 | 68.203 | 1.00 | 55.09 | 6 |
| ATOM | 176 | O | LEU | A | 28 | 4.938 | 71.047 | 68.383 | 1.00 | 54.89 | 8 |
| ATOM | 177 | N | GLU | A | 29 | 5.652 | 69.011 | 69.028 | 1.00 | 95.04 | 7 |
| ATOM | 178 | CA | GLU | A | 29 | 6.341 | 69.510 | 70.205 | 1.00 | 97.70 | 6 |
| ATOM | 179 | CB | GLU | A | 29 | 7.201 | 68.411 | 70.835 | 1.00 | 135.72 | 6 |
| ATOM | 180 | CG | GLU | A | 29 | 6.414 | 67.210 | 71.331 | 1.00 | 139.75 | 6 |
| ATOM | 181 | CD | GLU | A | 29 | 7.248 | 66.273 | 72.187 | 1.00 | 142.12 | 6 |
| ATOM | 182 | OE1 | GLU | A | 29 | 7.820 | 66.742 | 73.197 | 1.00 | 144.19 | 8 |
| ATOM | 183 | OE2 | GLU | A | 29 | 7.324 | 65.067 | 71.860 | 1.00 | 142.16 | 8 |
| ATOM | 184 | C | GLU | A | 29 | 7.221 | 70.683 | 69.806 | 1.00 | 98.60 | 6 |
| ATOM | 185 | O | GLU | A | 29 | 7.888 | 70.652 | 68.770 | 1.00 | 98.86 | 8 |
| ATOM | 186 | N | ARG | A | 30 | 7.206 | 71.729 | 70.620 | 1.00 | 49.18 | 7 |
| ATOM | 187 | CA | ARG | A | 30 | 8.021 | 72.900 | 70.349 | 1.00 | 48.17 | 6 |
| ATOM | 188 | CB | ARG | A | 30 | 8.029 | 73.811 | 71.573 | 1.00 | 135.75 | 6 |
| ATOM | 189 | CG | ARG | A | 30 | 7.710 | 73.099 | 72.883 | 1.00 | 138.35 | 6 |
| ATOM | 190 | CD | ARG | A | 30 | 6.214 | 72.839 | 73.015 | 1.00 | 140.35 | 6 |
| ATOM | 191 | NE | ARG | A | 30 | 5.858 | 72.349 | 74.342 | 1.00 | 141.60 | 7 |
| ATOM | 192 | CZ | ARG | A | 30 | 4.676 | 72.544 | 74.917 | 1.00 | 142.62 | 6 |
| ATOM | 193 | NH1 | ARG | A | 30 | 3.733 | 73.224 | 74.282 | 1.00 | 143.40 | 7 |
| ATOM | 194 | NH2 | ARG | A | 30 | 4.434 | 72.060 | 76.128 | 1.00 | 142.68 | 7 |
| ATOM | 195 | C | ARG | A | 30 | 9.449 | 72.499 | 69.996 | 1.00 | 47.03 | 6 |
| ATOM | 196 | O | ARG | A | 30 | 10.265 | 72.211 | 70.879 | 1.00 | 45.83 | 8 |
| ATOM | 197 | N | GLY | A | 31 | 9.746 | 72.474 | 68.704 | 1.00 | 86.21 | 7 |
| ATOM | 198 | CA | GLY | A | 31 | 11.082 | 72.113 | 68.289 | 1.00 | 85.50 | 6 |
| ATOM | 199 | C | GLY | A | 31 | 11.183 | 71.239 | 67.060 | 1.00 | 84.90 | 6 |
| ATOM | 200 | O | GLY | A | 31 | 11.913 | 71.569 | 66.127 | 1.00 | 85.07 | 8 |
| ATOM | 201 | N | PHE | A | 32 | 10.459 | 70.123 | 67.051 | 1.00 | 67.60 | 7 |
| ATOM | 202 | CA | PHE | A | 32 | 10.508 | 69.204 | 65.918 | 1.00 | 66.18 | 6 |
| ATOM | 203 | CB | PHE | A | 32 | 9.686 | 67.948 | 66.221 | 1.00 | 37.90 | 6 |
| ATOM | 204 | CG | PHE | A | 32 | 10.366 | 66.995 | 67.163 | 1.00 | 36.46 | 6 |
| ATOM | 205 | CD1 | PHE | A | 32 | 11.292 | 67.448 | 68.090 | 1.00 | 36.18 | 6 |
| ATOM | 206 | CD2 | PHE | A | 32 | 10.057 | 65.645 | 67.154 | 1.00 | 35.75 | 6 |
| ATOM | 207 | CE1 | PHE | A | 32 | 11.897 | 66.557 | 68.998 | 1.00 | 36.60 | 6 |
| ATOM | 208 | CE2 | PHE | A | 32 | 10.662 | 64.746 | 68.066 | 1.00 | 35.33 | 6 |
| ATOM | 209 | CZ | PHE | A | 32 | 11.573 | 65.200 | 68.979 | 1.00 | 35.10 | 6 |
| ATOM | 210 | C | PHE | A | 32 | 10.056 | 69.845 | 64.615 | 1.00 | 65.97 | 6 |
| ATOM | 211 | O | PHE | A | 32 | 10.196 | 69.254 | 63.546 | 1.00 | 65.84 | 8 |
| ATOM | 212 | N | GLY | A | 33 | 9.534 | 71.063 | 64.704 | 1.00 | 77.10 | 7 |
| ATOM | 213 | CA | GLY | A | 33 | 9.097 | 71.751 | 63.507 | 1.00 | 75.91 | 6 |
| ATOM | 214 | C | GLY | A | 33 | 10.094 | 71.568 | 62.376 | 1.00 | 75.60 | 6 |
| ATOM | 215 | O | GLY | A | 33 | 9.720 | 71.510 | 61.206 | 1.00 | 76.44 | 8 |
| ATOM | 216 | N | VAL | A | 34 | 11.372 | 71.467 | 62.725 | 1.00 | 52.80 | 7 |
| ATOM | 217 | CA | VAL | A | 34 | 12.410 | 71.289 | 61.724 | 1.00 | 51.83 | 6 |
| ATOM | 218 | CB | VAL | A | 34 | 13.501 | 72.380 | 61.800 | 1.00 | 59.73 | 6 |
| ATOM | 219 | CG1 | VAL | A | 34 | 12.909 | 73.749 | 61.479 | 1.00 | 60.67 | 6 |
| ATOM | 220 | CG2 | VAL | A | 34 | 14.141 | 72.363 | 63.168 | 1.00 | 61.37 | 6 |
| ATOM | 221 | C | VAL | A | 34 | 13.097 | 69.951 | 61.856 | 1.00 | 50.63 | 6 |
| ATOM | 222 | O | VAL | A | 34 | 13.579 | 69.422 | 60.863 | 1.00 | 51.62 | 8 |
| ATOM | 223 | N | THR | A | 35 | 13.154 | 69.392 | 63.063 | 1.00 | 36.90 | 7 |
| ATOM | 224 | CA | THR | A | 35 | 13.819 | 68.095 | 63.226 | 1.00 | 35.09 | 6 |
| ATOM | 225 | CB | THR | A | 35 | 13.474 | 67.373 | 64.559 | 1.00 | 52.73 | 6 |
| ATOM | 226 | OG1 | THR | A | 35 | 13.693 | 68.247 | 65.671 | 1.00 | 53.28 | 8 |
| ATOM | 227 | CG2 | THR | A | 35 | 14.371 | 66.168 | 64.733 | 1.00 | 52.98 | 6 |
| ATOM | 228 | C | THR | A | 35 | 13.309 | 67.240 | 62.088 | 1.00 | 32.97 | 6 |
| ATOM | 229 | O | THR | A | 35 | 14.026 | 66.415 | 61.536 | 1.00 | 32.07 | 8 |
| ATOM | 230 | N | LEU | A | 36 | 12.056 | 67.471 | 61.728 | 1.00 | 20.88 | 7 |
| ATOM | 231 | CA | LEU | A | 36 | 11.432 | 66.739 | 60.649 | 1.00 | 21.18 | 6 |
| ATOM | 232 | CB | LEU | A | 36 | 10.009 | 66.343 | 61.051 | 1.00 | 39.45 | 6 |
| ATOM | 233 | CG | LEU | A | 36 | 9.755 | 65.996 | 62.526 | 1.00 | 39.89 | 6 |
| ATOM | 234 | CD1 | LEU | A | 36 | 8.258 | 65.818 | 62.752 | 1.00 | 41.19 | 6 |
| ATOM | 235 | CD2 | LEU | A | 36 | 10.515 | 64.740 | 62.926 | 1.00 | 39.22 | 6 |
| ATOM | 236 | C | LEU | A | 36 | 11.397 | 67.668 | 59.442 | 1.00 | 21.63 | 6 |
| ATOM | 237 | O | LEU | A | 36 | 11.850 | 67.319 | 58.357 | 1.00 | 20.73 | 8 |
| ATOM | 238 | N | GLY | A | 37 | 10.875 | 68.868 | 59.666 | 1.00 | 54.11 | 7 |
| ATOM | 239 | CA | GLY | A | 37 | 10.742 | 69.862 | 58.616 | 1.00 | 56.99 | 6 |
| ATOM | 240 | C | GLY | A | 37 | 11.866 | 70.059 | 57.615 | 1.00 | 58.77 | 6 |
| ATOM | 241 | O | GLY | A | 37 | 11.597 | 70.381 | 56.463 | 1.00 | 60.64 | 8 |
| ATOM | 242 | N | ASN | A | 38 | 13.118 | 69.880 | 58.019 | 1.00 | 43.74 | 7 |
| ATOM | 243 | CA | ASN | A | 38 | 14.211 | 70.089 | 57.080 | 1.00 | 42.89 | 6 |
| ATOM | 244 | CB | ASN | A | 38 | 15.385 | 70.783 | 57.777 | 1.00 | 40.56 | 6 |
| ATOM | 245 | CG | ASN | A | 38 | 16.115 | 71.759 | 56.864 | 1.00 | 39.64 | 6 |
| ATOM | 246 | OD1 | ASN | A | 38 | 16.918 | 71.376 | 56.016 | 1.00 | 38.98 | 8 |
| ATOM | 247 | ND2 | ASN | A | 38 | 15.820 | 73.033 | 57.032 | 1.00 | 40.13 | 7 |
| ATOM | 248 | C | ASN | A | 38 | 14.693 | 68.826 | 56.362 | 1.00 | 42.40 | 6 |
| ATOM | 249 | O | ASN | A | 38 | 14.521 | 68.720 | 55.145 | 1.00 | 41.83 | 8 |
| ATOM | 250 | N | PRO | A | 39 | 15.282 | 67.851 | 57.094 | 1.00 | 40.48 | 7 |
| ATOM | 251 | CD | PRO | A | 39 | 15.267 | 67.679 | 58.555 | 1.00 | 16.97 | 6 |
| ATOM | 252 | CA | PRO | A | 39 | 15.770 | 66.620 | 56.463 | 1.00 | 41.24 | 6 |
| ATOM | 253 | CB | PRO | A | 39 | 15.764 | 65.618 | 57.618 | 1.00 | 16.06 | 6 |
| ATOM | 254 | CG | PRO | A | 39 | 16.131 | 66.439 | 58.747 | 1.00 | 15.86 | 6 |
| ATOM | 255 | C | PRO | A | 39 | 14.901 | 66.170 | 55.286 | 1.00 | 42.78 | 6 |
| ATOM | 256 | O | PRO | A | 39 | 15.411 | 65.649 | 54.290 | 1.00 | 42.03 | 8 |
| ATOM | 257 | N | LEU | A | 40 | 13.589 | 66.375 | 55.408 | 1.00 | 77.92 | 7 |
| ATOM | 258 | CA | LEU | A | 40 | 12.657 | 66.004 | 54.351 | 1.00 | 79.48 | 6 |
| ATOM | 259 | CB | LEU | A | 40 | 11.207 | 66.157 | 54.825 | 1.00 | 44.82 | 6 |
| ATOM | 260 | CG | LEU | A | 40 | 10.797 | 65.405 | 56.093 | 1.00 | 45.44 | 6 |
| ATOM | 261 | CD1 | LEU | A | 40 | 9.303 | 65.573 | 56.318 | 1.00 | 45.40 | 6 |
| ATOM | 262 | CD2 | LEU | A | 40 | 11.155 | 63.930 | 55.977 | 1.00 | 45.49 | 6 |
| ATOM | 263 | C | LEU | A | 40 | 12.906 | 66.921 | 53.165 | 1.00 | 80.46 | 6 |
| ATOM | 264 | O | LEU | A | 40 | 13.203 | 66.466 | 52.057 | 1.00 | 80.99 | 8 |
| ATOM | 265 | N | ARG | A | 41 | 12.793 | 68.221 | 53.408 | 1.00 | 78.27 | 7 |
| ATOM | 266 | CA | ARG | A | 41 | 13.010 | 69.201 | 52.359 | 1.00 | 77.86 | 6 |
| ATOM | 267 | CB | ARG | A | 41 | 12.950 | 70.615 | 52.948 | 1.00 | 71.76 | 6 |
| ATOM | 268 | CG | ARG | A | 41 | 12.558 | 71.705 | 51.953 | 1.00 | 73.75 | 6 |
| ATOM | 269 | CD | ARG | A | 41 | 13.741 | 72.275 | 51.194 | 1.00 | 74.31 | 6 |
| ATOM | 270 | NE | ARG | A | 41 | 14.589 | 73.075 | 52.066 | 1.00 | 76.34 | 7 |
| ATOM | 271 | CZ | ARG | A | 41 | 15.454 | 72.579 | 52.945 | 1.00 | 79.31 | 6 |
| ATOM | 272 | NH1 | ARG | A | 41 | 15.606 | 71.270 | 53.079 | 1.00 | 80.51 | 7 |
| ATOM | 273 | NH2 | ARG | A | 41 | 16.164 | 73.396 | 53.708 | 1.00 | 79.89 | 7 |
| ATOM | 274 | C | ARG | A | 41 | 14.376 | 68.910 | 51.748 | 1.00 | 76.52 | 6 |
| ATOM | 275 | O | ARG | A | 41 | 14.559 | 68.987 | 50.534 | 1.00 | 76.69 | 8 |
| ATOM | 276 | N | ARG | A | 42 | 15.333 | 68.559 | 52.598 | 1.00 | 55.69 | 7 |
| ATOM | 277 | CA | ARG | A | 42 | 16.665 | 68.228 | 52.117 | 1.00 | 54.23 | 6 |
| ATOM | 278 | CB | ARG | A | 42 | 17.559 | 67.759 | 53.266 | 1.00 | 43.27 | 6 |
| ATOM | 279 | CG | ARG | A | 42 | 17.965 | 68.846 | 54.226 | 1.00 | 43.46 | 6 |
| ATOM | 280 | CD | ARG | A | 42 | 19.175 | 69.576 | 53.713 | 1.00 | 42.80 | 6 |
| ATOM | 281 | NE | ARG | A | 42 | 20.400 | 68.918 | 54.144 | 1.00 | 41.66 | 7 |
| ATOM | 282 | CZ | ARG | A | 42 | 21.593 | 69.162 | 53.617 | 1.00 | 41.52 | 6 |
| ATOM | 283 | NH1 | ARG | A | 42 | 21.714 | 70.050 | 52.635 | 1.00 | 40.62 | 7 |
| ATOM | 284 | NH2 | ARG | A | 42 | 22.663 | 68.522 | 54.074 | 1.00 | 40.40 | 7 |
| ATOM | 285 | C | ARG | A | 42 | 16.490 | 67.094 | 51.127 | 1.00 | 52.39 | 6 |
| ATOM | 286 | O | ARG | A | 42 | 16.635 | 67.289 | 49.921 | 1.00 | 52.13 | 8 |
| ATOM | 287 | N | ILE | A | 43 | 16.150 | 65.914 | 51.650 | 1.00 | 28.41 | 7 |
| ATOM | 288 | CA | ILE | A | 43 | 15.961 | 64.731 | 50.827 | 1.00 | 25.56 | 6 |
| ATOM | 289 | CB | ILE | A | 43 | 15.265 | 63.585 | 51.616 | 1.00 | 20.13 | 6 |
| ATOM | 290 | CG2 | ILE | A | 43 | 15.289 | 62.291 | 50.806 | 1.00 | 18.45 | 6 |
| ATOM | 291 | CG1 | ILE | A | 43 | 16.002 | 63.313 | 52.926 | 1.00 | 19.25 | 6 |
| ATOM | 292 | CD1 | ILE | A | 43 | 17.358 | 62.780 | 52.730 | 1.00 | 20.64 | 6 |
| ATOM | 293 | C | ILE | A | 43 | 15.122 | 65.096 | 49.611 | 1.00 | 24.61 | 6 |
| ATOM | 294 | O | ILE | A | 43 | 15.596 | 64.997 | 48.483 | 1.00 | 23.15 | 8 |
| ATOM | 295 | N | LEU | A | 44 | 13.891 | 65.538 | 49.834 | 1.00 | 60.43 | 7 |
| ATOM | 296 | CA | LEU | A | 44 | 13.022 | 65.900 | 48.721 | 1.00 | 62.04 | 6 |
| ATOM | 297 | CB | LEU | A | 44 | 11.928 | 66.843 | 49.205 | 1.00 | 32.70 | 6 |
| ATOM | 298 | CG | LEU | A | 44 | 10.914 | 66.119 | 50.078 | 1.00 | 31.74 | 6 |
| ATOM | 299 | CD1 | LEU | A | 44 | 10.156 | 67.122 | 50.898 | 1.00 | 32.66 | 6 |
| ATOM | 300 | CD2 | LEU | A | 44 | 9.969 | 65.310 | 49.219 | 1.00 | 31.99 | 6 |
| ATOM | 301 | C | LEU | A | 44 | 13.770 | 66.526 | 47.539 | 1.00 | 62.79 | 6 |
| ATOM | 302 | O | LEU | A | 44 | 13.569 | 66.130 | 46.387 | 1.00 | 63.37 | 8 |
| ATOM | 303 | N | LEU | A | 45 | 14.645 | 67.487 | 47.828 | 1.00 | 54.74 | 7 |
| ATOM | 304 | CA | LEU | A | 45 | 15.411 | 68.169 | 46.789 | 1.00 | 52.16 | 6 |
| ATOM | 305 | CB | LEU | A | 45 | 16.062 | 69.423 | 47.363 | 1.00 | 13.87 | 6 |
| ATOM | 306 | CG | LEU | A | 45 | 15.152 | 70.604 | 47.623 | 1.00 | 13.87 | 6 |
| ATOM | 307 | CD1 | LEU | A | 45 | 15.843 | 71.586 | 48.516 | 1.00 | 13.87 | 6 |
| ATOM | 308 | CD2 | LEU | A | 45 | 14.792 | 71.238 | 46.320 | 1.00 | 13.87 | 6 |
| ATOM | 309 | C | LEU | A | 45 | 16.494 | 67.306 | 46.167 | 1.00 | 52.20 | 6 |
| ATOM | 310 | O | LEU | A | 45 | 16.788 | 67.441 | 44.980 | 1.00 | 51.60 | 8 |
| ATOM | 311 | N | SER | A | 46 | 17.074 | 66.423 | 46.977 | 1.00 | 47.04 | 7 |
| ATOM | 312 | CA | SER | A | 46 | 18.169 | 65.550 | 46.556 | 1.00 | 47.85 | 6 |
| ATOM | 313 | CB | SER | A | 46 | 19.098 | 65.282 | 47.734 | 1.00 | 62.29 | 6 |
| ATOM | 314 | OG | SER | A | 46 | 19.440 | 66.478 | 48.399 | 1.00 | 64.36 | 8 |
| ATOM | 315 | C | SER | A | 46 | 17.793 | 64.207 | 45.965 | 1.00 | 47.22 | 6 |
| ATOM | 316 | O | SER | A | 46 | 17.335 | 64.098 | 44.833 | 1.00 | 47.52 | 8 |
| ATOM | 317 | N | SER | A | 47 | 18.015 | 63.171 | 46.754 | 1.00 | 19.54 | 7 |
| ATOM | 318 | CA | SER | A | 47 | 17.740 | 61.819 | 46.314 | 1.00 | 20.83 | 6 |
| ATOM | 319 | CB | SER | A | 47 | 18.339 | 60.840 | 47.330 | 1.00 | 47.90 | 6 |
| ATOM | 320 | OG | SER | A | 47 | 19.636 | 61.245 | 47.740 | 1.00 | 48.74 | 8 |
| ATOM | 321 | C | SER | A | 47 | 16.240 | 61.523 | 46.111 | 1.00 | 20.87 | 6 |
| ATOM | 322 | O | SER | A | 47 | 15.415 | 61.785 | 46.986 | 1.00 | 21.06 | 8 |
| ATOM | 323 | N | ILE | A | 48 | 15.900 | 60.977 | 44.953 | 1.00 | 46.59 | 7 |
| ATOM | 324 | CA | ILE | A | 48 | 14.528 | 60.621 | 44.624 | 1.00 | 48.06 | 6 |
| ATOM | 325 | CB | ILE | A | 48 | 13.512 | 61.749 | 44.878 | 1.00 | 13.87 | 6 |
| ATOM | 326 | CG2 | ILE | A | 48 | 12.415 | 61.715 | 43.809 | 1.00 | 13.87 | 6 |
| ATOM | 327 | CG1 | ILE | A | 48 | 12.859 | 61.582 | 46.244 | 1.00 | 13.87 | 6 |
| ATOM | 328 | CD1 | ILE | A | 48 | 11.777 | 62.610 | 46.523 | 1.00 | 13.87 | 6 |
| ATOM | 329 | C | ILE | A | 48 | 14.472 | 60.333 | 43.151 | 1.00 | 50.88 | 6 |
| ATOM | 330 | O | ILE | A | 48 | 14.926 | 61.135 | 42.337 | 1.00 | 52.63 | 8 |
| ATOM | 331 | N | PRO | A | 49 | 13.902 | 59.185 | 42.783 | 1.00 | 39.58 | 7 |
| ATOM | 332 | CD | PRO | A | 49 | 13.394 | 58.110 | 43.655 | 1.00 | 145.29 | 6 |
| ATOM | 333 | CA | PRO | A | 49 | 13.797 | 58.814 | 41.377 | 1.00 | 39.89 | 6 |
| ATOM | 334 | CB | PRO | A | 49 | 12.979 | 57.525 | 41.428 | 1.00 | 145.15 | 6 |
| ATOM | 335 | CG | PRO | A | 49 | 13.408 | 56.916 | 42.729 | 1.00 | 146.08 | 6 |
| ATOM | 336 | C | PRO | A | 49 | 13.122 | 59.903 | 40.544 | 1.00 | 40.28 | 6 |
| ATOM | 337 | O | PRO | A | 49 | 12.250 | 60.639 | 41.028 | 1.00 | 39.98 | 8 |
| ATOM | 338 | N | GLY | A | 50 | 13.547 | 59.985 | 39.286 | 1.00 | 41.27 | 7 |
| ATOM | 339 | CA | GLY | A | 50 | 13.005 | 60.947 | 38.349 | 1.00 | 40.92 | 6 |
| ATOM | 340 | C | GLY | A | 50 | 13.190 | 60.493 | 36.911 | 1.00 | 39.90 | 6 |
| ATOM | 341 | O | GLY | A | 50 | 13.762 | 59.439 | 36.615 | 1.00 | 38.85 | 8 |
| ATOM | 342 | N | THR | A | 51 | 12.704 | 61.315 | 36.001 | 1.00 | 41.55 | 7 |
| ATOM | 343 | CA | THR | A | 51 | 12.794 | 61.010 | 34.593 | 1.00 | 42.82 | 6 |
| ATOM | 344 | CB | THR | A | 51 | 11.383 | 60.828 | 33.998 | 1.00 | 61.58 | 6 |
| ATOM | 345 | OG1 | THR | A | 51 | 10.709 | 62.095 | 33.965 | 1.00 | 63.80 | 8 |
| ATOM | 346 | CG2 | THR | A | 51 | 10.569 | 59.864 | 34.849 | 1.00 | 62.73 | 6 |
| ATOM | 347 | C | THR | A | 51 | 13.482 | 62.176 | 33.890 | 1.00 | 41.48 | 6 |
| ATOM | 348 | O | THR | A | 51 | 13.023 | 63.323 | 33.985 | 1.00 | 40.65 | 8 |
| ATOM | 349 | N | ALA | A | 52 | 14.584 | 61.893 | 33.194 | 1.00 | 29.66 | 7 |
| ATOM | 350 | CA | ALA | A | 52 | 15.298 | 62.941 | 32.469 | 1.00 | 30.42 | 6 |
| ATOM | 351 | CB | ALA | A | 52 | 16.441 | 63.462 | 33.312 | 1.00 | 77.76 | 6 |
| ATOM | 352 | C | ALA | A | 52 | 15.823 | 62.519 | 31.106 | 1.00 | 29.19 | 6 |
| ATOM | 353 | O | ALA | A | 52 | 16.153 | 61.364 | 30.883 | 1.00 | 27.97 | 8 |
| ATOM | 354 | N | VAL | A | 53 | 15.887 | 63.478 | 30.195 | 1.00 | 33.16 | 7 |
| ATOM | 355 | CA | VAL | A | 53 | 16.400 | 63.247 | 28.849 | 1.00 | 33.88 | 6 |
| ATOM | 356 | CB | VAL | A | 53 | 16.381 | 64.569 | 28.066 | 1.00 | 31.89 | 6 |
| ATOM | 357 | CG1 | VAL | A | 53 | 16.599 | 64.340 | 26.574 | 1.00 | 31.71 | 6 |
| ATOM | 358 | CG2 | VAL | A | 53 | 15.084 | 65.270 | 28.335 | 1.00 | 31.88 | 6 |
| ATOM | 359 | C | VAL | A | 53 | 17.856 | 62.776 | 28.970 | 1.00 | 34.18 | 6 |
| ATOM | 360 | O | VAL | A | 53 | 18.671 | 63.447 | 29.595 | 1.00 | 34.70 | 8 |
| ATOM | 361 | N | THR | A | 54 | 18.212 | 61.645 | 28.382 | 1.00 | 53.17 | 7 |
| ATOM | 362 | CA | THR | A | 54 | 19.596 | 61.222 | 28.518 | 1.00 | 55.64 | 6 |
| ATOM | 363 | CB | THR | A | 54 | 19.689 | 59.742 | 28.906 | 1.00 | 54.68 | 6 |
| ATOM | 364 | OG1 | THR | A | 54 | 19.168 | 59.570 | 30.227 | 1.00 | 55.11 | 8 |
| ATOM | 365 | CG2 | THR | A | 54 | 21.141 | 59.269 | 28.885 | 1.00 | 55.71 | 6 |
| ATOM | 366 | C | THR | A | 54 | 20.464 | 61.470 | 27.294 | 1.00 | 56.10 | 6 |
| ATOM | 367 | O | THR | A | 54 | 21.642 | 61.817 | 27.437 | 1.00 | 56.71 | 8 |
| ATOM | 368 | N | SER | A | 55 | 19.881 | 61.302 | 26.102 | 1.00 | 42.59 | 7 |
| ATOM | 369 | CA | SER | A | 55 | 20.595 | 61.492 | 24.830 | 1.00 | 43.27 | 6 |
| ATOM | 370 | CB | SER | A | 55 | 21.444 | 60.253 | 24.536 | 1.00 | 30.83 | 6 |
| ATOM | 371 | OG | SER | A | 55 | 22.262 | 60.453 | 23.409 | 1.00 | 31.95 | 8 |
| ATOM | 372 | C | SER | A | 55 | 19.624 | 61.754 | 23.669 | 1.00 | 42.60 | 6 |
| ATOM | 373 | O | SER | A | 55 | 18.516 | 61.222 | 23.641 | 1.00 | 41.99 | 8 |
| ATOM | 374 | N | VAL | A | 56 | 20.042 | 62.567 | 22.707 | 1.00 | 49.56 | 7 |
| ATOM | 375 | CA | VAL | A | 56 | 19.167 | 62.893 | 21.585 | 1.00 | 51.39 | 6 |
| ATOM | 376 | CB | VAL | A | 56 | 18.463 | 64.291 | 21.818 | 1.00 | 18.02 | 6 |
| ATOM | 377 | CG1 | VAL | A | 56 | 18.759 | 64.775 | 23.223 | 1.00 | 17.77 | 6 |
| ATOM | 378 | CG2 | VAL | A | 56 | 18.899 | 65.343 | 20.784 | 1.00 | 15.68 | 6 |
| ATOM | 379 | C | VAL | A | 56 | 19.843 | 62.892 | 20.219 | 1.00 | 52.77 | 6 |
| ATOM | 380 | O | VAL | A | 56 | 20.836 | 63.586 | 20.009 | 1.00 | 53.75 | 8 |
| ATOM | 381 | N | TYR | A | 57 | 19.306 | 62.103 | 19.292 | 1.00 | 49.16 | 7 |
| ATOM | 382 | CA | TYR | A | 57 | 19.842 | 62.065 | 17.935 | 1.00 | 50.62 | 6 |
| ATOM | 383 | CB | TYR | A | 57 | 19.973 | 60.627 | 17.431 | 1.00 | 56.89 | 6 |
| ATOM | 384 | CG | TYR | A | 57 | 20.071 | 60.507 | 15.908 | 1.00 | 55.88 | 6 |
| ATOM | 385 | CD1 | TYR | A | 57 | 21.230 | 60.024 | 15.295 | 1.00 | 55.45 | 6 |
| ATOM | 386 | CE1 | TYR | A | 57 | 21.305 | 59.879 | 13.911 | 1.00 | 54.69 | 6 |
| ATOM | 387 | CD2 | TYR | A | 57 | 18.991 | 60.848 | 15.084 | 1.00 | 55.26 | 6 |
| ATOM | 388 | CE2 | TYR | A | 57 | 19.061 | 60.710 | 13.710 | 1.00 | 54.91 | 6 |
| ATOM | 389 | CZ | TYR | A | 57 | 20.216 | 60.222 | 13.130 | 1.00 | 54.94 | 6 |
| ATOM | 390 | OH | TYR | A | 57 | 20.270 | 60.047 | 11.770 | 1.00 | 55.85 | 8 |
| ATOM | 391 | C | TYR | A | 57 | 18.933 | 62.836 | 16.977 | 1.00 | 51.34 | 6 |
| ATOM | 392 | O | TYR | A | 57 | 17.713 | 62.680 | 16.997 | 1.00 | 51.30 | 8 |
| ATOM | 393 | N | ILE | A | 58 | 19.529 | 63.651 | 16.120 | 1.00 | 48.33 | 7 |
| ATOM | 394 | CA | ILE | A | 58 | 18.738 | 64.390 | 15.160 | 1.00 | 50.45 | 6 |
| ATOM | 395 | CB | ILE | A | 58 | 18.904 | 65.906 | 15.341 | 1.00 | 43.10 | 6 |
| ATOM | 396 | CG2 | ILE | A | 58 | 18.130 | 66.651 | 14.254 | 1.00 | 42.04 | 6 |
| ATOM | 397 | CG1 | ILE | A | 58 | 18.379 | 66.317 | 16.712 | 1.00 | 42.84 | 6 |
| ATOM | 398 | CD1 | ILE | A | 58 | 18.051 | 67.801 | 16.816 | 1.00 | 44.49 | 6 |
| ATOM | 399 | C | ILE | A | 58 | 19.107 | 63.991 | 13.733 | 1.00 | 51.73 | 6 |
| ATOM | 400 | O | ILE | A | 58 | 20.239 | 63.590 | 13.457 | 1.00 | 50.59 | 8 |
| ATOM | 401 | N | GLU | A | 59 | 18.127 | 64.110 | 12.841 | 1.00 | 92.68 | 7 |
| ATOM | 402 | CA | GLU | A | 59 | 18.267 | 63.761 | 11.430 | 1.00 | 95.41 | 6 |
| ATOM | 403 | CB | GLU | A | 59 | 17.209 | 64.501 | 10.598 | 1.00 | 103.73 | 6 |
| ATOM | 404 | CG | GLU | A | 59 | 17.103 | 64.033 | 9.140 | 1.00 | 105.12 | 6 |
| ATOM | 405 | CD | GLU | A | 59 | 16.435 | 62.663 | 8.974 | 1.00 | 106.04 | 6 |
| ATOM | 406 | OE1 | GLU | A | 59 | 16.917 | 61.675 | 9.571 | 1.00 | 106.23 | 8 |
| ATOM | 407 | OE2 | GLU | A | 59 | 15.429 | 62.569 | 8.234 | 1.00 | 106.36 | 8 |
| ATOM | 408 | C | GLU | A | 59 | 19.646 | 64.019 | 10.836 | 1.00 | 95.22 | 6 |
| ATOM | 409 | O | GLU | A | 59 | 20.574 | 63.238 | 11.038 | 1.00 | 95.27 | 8 |
| ATOM | 410 | N | ASP | A | 60 | 19.776 | 65.119 | 10.104 | 1.00 | 54.07 | 7 |
| ATOM | 411 | CA | ASP | A | 60 | 21.039 | 65.455 | 9.451 | 1.00 | 54.95 | 6 |
| ATOM | 412 | CB | ASP | A | 60 | 20.755 | 66.216 | 8.148 | 1.00 | 121.45 | 6 |
| ATOM | 413 | CG | ASP | A | 60 | 19.988 | 65.380 | 7.130 | 1.00 | 123.20 | 6 |
| ATOM | 414 | OD1 | ASP | A | 60 | 20.519 | 64.343 | 6.672 | 1.00 | 124.19 | 8 |
| ATOM | 415 | OD2 | ASP | A | 60 | 18.851 | 65.760 | 6.778 | 1.00 | 123.59 | 8 |
| ATOM | 416 | C | ASP | A | 60 | 22.016 | 66.261 | 10.311 | 1.00 | 54.33 | 6 |
| ATOM | 417 | O | ASP | A | 60 | 22.484 | 67.321 | 9.896 | 1.00 | 54.17 | 8 |
| ATOM | 418 | N | VAL | A | 61 | 22.351 | 65.757 | 11.496 | 1.00 | 54.31 | 7 |
| ATOM | 419 | CA | VAL | A | 61 | 23.268 | 66.491 | 12.363 | 1.00 | 55.07 | 6 |
| ATOM | 420 | CB | VAL | A | 61 | 22.507 | 67.192 | 13.511 | 1.00 | 62.32 | 6 |
| ATOM | 421 | CG1 | VAL | A | 61 | 23.498 | 67.801 | 14.504 | 1.00 | 62.29 | 6 |
| ATOM | 422 | CG2 | VAL | A | 61 | 21.600 | 68.275 | 12.939 | 1.00 | 62.30 | 6 |
| ATOM | 423 | C | VAL | A | 61 | 24.389 | 65.663 | 12.970 | 1.00 | 54.97 | 6 |
| ATOM | 424 | O | VAL | A | 61 | 24.152 | 64.798 | 13.808 | 1.00 | 54.94 | 8 |
| ATOM | 425 | N | LEU | A | 62 | 25.614 | 65.965 | 12.552 | 1.00 | 73.57 | 7 |
| ATOM | 426 | CA | LEU | A | 62 | 26.790 | 65.274 | 13.040 | 1.00 | 74.33 | 6 |
| ATOM | 427 | CB | LEU | A | 62 | 28.035 | 65.900 | 12.438 | 1.00 | 68.22 | 6 |
| ATOM | 428 | CG | LEU | A | 62 | 28.478 | 65.236 | 11.138 | 1.00 | 68.33 | 6 |
| ATOM | 429 | CD1 | LEU | A | 62 | 29.514 | 66.087 | 10.432 | 1.00 | 68.18 | 6 |
| ATOM | 430 | CD2 | LEU | A | 62 | 29.033 | 63.854 | 11.463 | 1.00 | 68.59 | 6 |
| ATOM | 431 | C | LEU | A | 62 | 26.891 | 65.266 | 14.554 | 1.00 | 74.75 | 6 |
| ATOM | 432 | O | LEU | A | 62 | 26.801 | 64.211 | 15.162 | 1.00 | 75.89 | 8 |
| ATOM | 433 | N | HIS | A | 63 | 27.095 | 66.430 | 15.162 | 1.00 | 68.14 | 7 |
| ATOM | 434 | CA | HIS | A | 63 | 27.188 | 66.533 | 16.622 | 1.00 | 68.73 | 6 |
| ATOM | 435 | CB | HIS | A | 63 | 28.612 | 66.247 | 17.112 | 1.00 | 109.32 | 6 |
| ATOM | 436 | CG | HIS | A | 63 | 28.817 | 64.842 | 17.598 | 1.00 | 111.08 | 6 |
| ATOM | 437 | CD2 | HIS | A | 63 | 28.015 | 63.753 | 17.545 | 1.00 | 111.66 | 6 |
| ATOM | 438 | ND1 | HIS | A | 63 | 29.970 | 64.436 | 18.234 | 1.00 | 112.16 | 7 |
| ATOM | 439 | CE1 | HIS | A | 63 | 29.868 | 63.157 | 18.552 | 1.00 | 112.77 | 6 |
| ATOM | 440 | NE2 | HIS | A | 63 | 28.692 | 62.719 | 18.144 | 1.00 | 112.76 | 7 |
| ATOM | 441 | C | HIS | A | 63 | 26.760 | 67.921 | 17.067 | 1.00 | 67.23 | 6 |
| ATOM | 442 | O | HIS | A | 63 | 26.497 | 68.774 | 16.225 | 1.00 | 67.52 | 8 |
| ATOM | 443 | N | GLU | A | 64 | 26.687 | 68.145 | 18.380 | 1.00 | 95.10 | 7 |
| ATOM | 444 | CA | GLU | A | 64 | 26.269 | 69.438 | 18.922 | 1.00 | 93.64 | 6 |
| ATOM | 445 | CB | GLU | A | 64 | 26.480 | 69.480 | 20.439 | 1.00 | 79.44 | 6 |
| ATOM | 446 | CG | GLU | A | 64 | 25.731 | 70.615 | 21.143 | 1.00 | 80.88 | 6 |
| ATOM | 447 | CD | GLU | A | 64 | 25.248 | 70.242 | 22.554 | 1.00 | 81.25 | 6 |
| ATOM | 448 | OE1 | GLU | A | 64 | 25.604 | 69.147 | 23.047 | 1.00 | 81.28 | 8 |
| ATOM | 449 | OE2 | GLU | A | 64 | 24.508 | 71.045 | 23.174 | 1.00 | 81.64 | 8 |
| ATOM | 450 | C | GLU | A | 64 | 27.054 | 70.548 | 18.240 | 1.00 | 91.41 | 6 |
| ATOM | 451 | O | GLU | A | 64 | 28.046 | 70.278 | 17.562 | 1.00 | 92.49 | 8 |
| ATOM | 452 | N | PHE | A | 65 | 26.617 | 71.791 | 18.420 | 1.00 | 22.51 | 7 |
| ATOM | 453 | CA | PHE | A | 65 | 27.266 | 72.948 | 17.782 | 1.00 | 20.38 | 6 |
| ATOM | 454 | CB | PHE | A | 65 | 28.801 | 72.924 | 17.954 | 1.00 | 28.12 | 6 |
| ATOM | 455 | CG | PHE | A | 65 | 29.236 | 72.710 | 19.363 | 1.00 | 24.54 | 6 |
| ATOM | 456 | CD1 | PHE | A | 65 | 29.535 | 71.434 | 19.827 | 1.00 | 22.93 | 6 |
| ATOM | 457 | CD2 | PHE | A | 65 | 29.224 | 73.757 | 20.267 | 1.00 | 23.17 | 6 |
| ATOM | 458 | CE1 | PHE | A | 65 | 29.804 | 71.197 | 21.170 | 1.00 | 21.54 | 6 |
| ATOM | 459 | CE2 | PHE | A | 65 | 29.489 | 73.530 | 21.616 | 1.00 | 21.40 | 6 |
| ATOM | 460 | CZ | PHE | A | 65 | 29.777 | 72.247 | 22.062 | 1.00 | 20.54 | 6 |
| ATOM | 461 | C | PHE | A | 65 | 26.924 | 72.930 | 16.294 | 1.00 | 19.54 | 6 |
| ATOM | 462 | O | PHE | A | 65 | 27.787 | 73.189 | 15.447 | 1.00 | 17.93 | 8 |
| ATOM | 463 | N | SER | A | 66 | 25.660 | 72.621 | 15.990 | 1.00 | 107.08 | 7 |
| ATOM | 464 | CA | SER | A | 66 | 25.167 | 72.543 | 14.614 | 1.00 | 108.68 | 6 |
| ATOM | 465 | CB | SER | A | 66 | 24.507 | 71.186 | 14.365 | 1.00 | 131.29 | 6 |
| ATOM | 466 | OG | SER | A | 66 | 25.451 | 70.136 | 14.446 | 1.00 | 132.76 | 8 |
| ATOM | 467 | C | SER | A | 66 | 24.184 | 73.648 | 14.239 | 1.00 | 107.83 | 6 |
| ATOM | 468 | O | SER | A | 66 | 24.159 | 74.706 | 14.859 | 1.00 | 109.21 | 8 |
| ATOM | 469 | N | THR | A | 67 | 23.368 | 73.384 | 13.223 | 1.00 | 37.78 | 7 |
| ATOM | 470 | CA | THR | A | 67 | 22.399 | 74.358 | 12.731 | 1.00 | 37.55 | 6 |
| ATOM | 471 | CB | THR | A | 67 | 23.084 | 75.605 | 12.161 | 1.00 | 38.25 | 6 |
| ATOM | 472 | OG1 | THR | A | 67 | 22.154 | 76.305 | 11.322 | 1.00 | 36.51 | 8 |
| ATOM | 473 | CG2 | THR | A | 67 | 24.319 | 75.214 | 11.337 | 1.00 | 36.90 | 6 |
| ATOM | 474 | C | THR | A | 67 | 21.540 | 73.785 | 11.614 | 1.00 | 37.84 | 6 |
| ATOM | 475 | O | THR | A | 67 | 21.777 | 74.052 | 10.435 | 1.00 | 38.15 | 8 |
| ATOM | 476 | N | ILE | A | 68 | 20.535 | 73.011 | 12.005 | 1.00 | 72.99 | 7 |
| ATOM | 477 | CA | ILE | A | 68 | 19.602 | 72.365 | 11.088 | 1.00 | 74.27 | 6 |
| ATOM | 478 | CB | ILE | A | 68 | 18.499 | 71.679 | 11.878 | 1.00 | 43.22 | 6 |
| ATOM | 479 | CG2 | ILE | A | 68 | 17.632 | 70.891 | 10.946 | 1.00 | 43.12 | 6 |
| ATOM | 480 | CG1 | ILE | A | 68 | 19.109 | 70.788 | 12.957 | 1.00 | 42.80 | 6 |
| ATOM | 481 | CD1 | ILE | A | 68 | 18.125 | 70.363 | 14.024 | 1.00 | 42.11 | 6 |
| ATOM | 482 | C | ILE | A | 68 | 18.915 | 73.306 | 10.093 | 1.00 | 74.17 | 6 |
| ATOM | 483 | O | ILE | A | 68 | 17.998 | 74.036 | 10.470 | 1.00 | 75.51 | 8 |
| ATOM | 484 | N | PRO | A | 69 | 19.335 | 73.293 | 8.808 | 1.00 | 47.83 | 7 |
| ATOM | 485 | CD | PRO | A | 69 | 20.552 | 72.652 | 8.266 | 1.00 | 30.92 | 6 |
| ATOM | 486 | CA | PRO | A | 69 | 18.723 | 74.161 | 7.790 | 1.00 | 48.16 | 6 |
| ATOM | 487 | CB | PRO | A | 69 | 19.365 | 73.676 | 6.497 | 1.00 | 30.11 | 6 |
| ATOM | 488 | CG | PRO | A | 69 | 20.781 | 73.419 | 6.965 | 1.00 | 30.52 | 6 |
| ATOM | 489 | C | PRO | A | 69 | 17.204 | 74.102 | 7.739 | 1.00 | 46.97 | 6 |
| ATOM | 490 | O | PRO | A | 69 | 16.616 | 73.020 | 7.661 | 1.00 | 46.45 | 8 |
| ATOM | 491 | N | GLY | A | 70 | 16.586 | 75.283 | 7.799 | 1.00 | 82.64 | 7 |
| ATOM | 492 | CA | GLY | A | 70 | 15.138 | 75.393 | 7.753 | 1.00 | 83.42 | 6 |
| ATOM | 493 | C | GLY | A | 70 | 14.471 | 75.570 | 9.106 | 1.00 | 83.11 | 6 |
| ATOM | 494 | O | GLY | A | 70 | 13.249 | 75.722 | 9.188 | 1.00 | 83.49 | 8 |
| ATOM | 495 | N | VAL | A | 71 | 15.266 | 75.548 | 10.170 | 1.00 | 25.92 | 7 |
| ATOM | 496 | CA | VAL | A | 71 | 14.738 | 75.700 | 11.519 | 1.00 | 25.88 | 6 |
| ATOM | 497 | CB | VAL | A | 71 | 15.182 | 74.528 | 12.462 | 1.00 | 69.23 | 6 |
| ATOM | 498 | CG1 | VAL | A | 71 | 14.438 | 74.612 | 13.787 | 1.00 | 69.55 | 6 |
| ATOM | 499 | CG2 | VAL | A | 71 | 14.923 | 73.176 | 11.811 | 1.00 | 69.40 | 6 |
| ATOM | 500 | C | VAL | A | 71 | 15.254 | 76.995 | 12.109 | 1.00 | 24.57 | 6 |
| ATOM | 501 | O | VAL | A | 71 | 16.385 | 77.396 | 11.857 | 1.00 | 23.69 | 8 |
| ATOM | 502 | N | LYS | A | 72 | 14.422 | 77.642 | 12.910 | 1.00 | 61.33 | 7 |
| ATOM | 503 | CA | LYS | A | 72 | 14.794 | 78.891 | 13.559 | 1.00 | 61.86 | 6 |
| ATOM | 504 | CB | LYS | A | 72 | 13.567 | 79.514 | 14.227 | 1.00 | 80.38 | 6 |
| ATOM | 505 | CG | LYS | A | 72 | 13.835 | 80.872 | 14.830 | 1.00 | 81.39 | 6 |
| ATOM | 506 | CD | LYS | A | 72 | 12.590 | 81.460 | 15.447 | 1.00 | 82.45 | 6 |
| ATOM | 507 | CE | LYS | A | 72 | 12.867 | 82.860 | 15.951 | 1.00 | 83.55 | 6 |
| ATOM | 508 | NZ | LYS | A | 72 | 11.769 | 83.344 | 16.827 | 1.00 | 85.83 | 7 |
| ATOM | 509 | C | LYS | A | 72 | 15.885 | 78.689 | 14.601 | 1.00 | 61.01 | 6 |
| ATOM | 510 | O | LYS | A | 72 | 16.965 | 79.264 | 14.503 | 1.00 | 60.86 | 8 |
| ATOM | 511 | N | GLU | A | 73 | 15.580 | 77.860 | 15.593 | 1.00 | 37.71 | 7 |
| ATOM | 512 | CA | GLU | A | 73 | 16.475 | 77.557 | 16.702 | 1.00 | 38.31 | 6 |
| ATOM | 513 | CB | GLU | A | 73 | 15.911 | 76.368 | 17.489 | 1.00 | 60.62 | 6 |
| ATOM | 514 | CG | GLU | A | 73 | 14.399 | 76.363 | 17.551 | 1.00 | 63.75 | 6 |
| ATOM | 515 | CD | GLU | A | 73 | 13.844 | 77.717 | 17.925 | 1.00 | 65.67 | 6 |
| ATOM | 516 | OE1 | GLU | A | 73 | 13.822 | 78.040 | 19.130 | 1.00 | 66.19 | 8 |
| ATOM | 517 | OE2 | GLU | A | 73 | 13.444 | 78.467 | 17.009 | 1.00 | 67.76 | 8 |
| ATOM | 518 | C | GLU | A | 73 | 17.945 | 77.287 | 16.367 | 1.00 | 37.99 | 6 |
| ATOM | 519 | O | GLU | A | 73 | 18.520 | 77.864 | 15.445 | 1.00 | 38.72 | 8 |
| ATOM | 520 | N | ASP | A | 74 | 18.534 | 76.388 | 17.151 | 1.00 | 82.96 | 7 |
| ATOM | 521 | CA | ASP | A | 74 | 19.935 | 75.994 | 17.037 | 1.00 | 81.66 | 6 |
| ATOM | 522 | CB | ASP | A | 74 | 20.808 | 77.116 | 17.581 | 1.00 | 54.13 | 6 |
| ATOM | 523 | CG | ASP | A | 74 | 22.192 | 76.656 | 17.930 | 1.00 | 52.60 | 6 |
| ATOM | 524 | OD1 | ASP | A | 74 | 22.333 | 75.533 | 18.467 | 1.00 | 50.96 | 8 |
| ATOM | 525 | OD2 | ASP | A | 74 | 23.137 | 77.433 | 17.683 | 1.00 | 52.36 | 8 |
| ATOM | 526 | C | ASP | A | 74 | 20.085 | 74.747 | 17.903 | 1.00 | 80.79 | 6 |
| ATOM | 527 | O | ASP | A | 74 | 19.780 | 74.783 | 19.090 | 1.00 | 81.18 | 8 |
| ATOM | 528 | N | VAL | A | 75 | 20.584 | 73.661 | 17.326 | 1.00 | 40.61 | 7 |
| ATOM | 529 | CA | VAL | A | 75 | 20.710 | 72.400 | 18.055 | 1.00 | 38.44 | 6 |
| ATOM | 530 | CB | VAL | A | 75 | 21.747 | 71.469 | 17.412 | 1.00 | 21.23 | 6 |
| ATOM | 531 | CG1 | VAL | A | 75 | 21.643 | 70.059 | 18.019 | 1.00 | 17.92 | 6 |
| ATOM | 532 | CG2 | VAL | A | 75 | 21.539 | 71.446 | 15.904 | 1.00 | 20.66 | 6 |
| ATOM | 533 | C | VAL | A | 75 | 21.040 | 72.538 | 19.530 | 1.00 | 38.36 | 6 |
| ATOM | 534 | O | VAL | A | 75 | 20.438 | 71.853 | 20.362 | 1.00 | 38.29 | 8 |
| ATOM | 535 | N | VAL | A | 76 | 21.987 | 73.411 | 19.864 | 1.00 | 74.82 | 7 |
| ATOM | 536 | CA | VAL | A | 76 | 22.347 | 73.584 | 21.267 | 1.00 | 75.22 | 6 |
| ATOM | 537 | CB | VAL | A | 76 | 23.393 | 74.690 | 21.484 | 1.00 | 34.21 | 6 |
| ATOM | 538 | CG1 | VAL | A | 76 | 23.993 | 74.554 | 22.876 | 1.00 | 34.49 | 6 |
| ATOM | 539 | CG2 | VAL | A | 76 | 24.480 | 74.592 | 20.448 | 1.00 | 34.06 | 6 |
| ATOM | 540 | C | VAL | A | 76 | 21.067 | 73.977 | 21.967 | 1.00 | 76.36 | 6 |
| ATOM | 541 | O | VAL | A | 76 | 20.603 | 73.271 | 22.866 | 1.00 | 76.45 | 8 |
| ATOM | 542 | N | GLU | A | 77 | 20.494 | 75.098 | 21.534 | 1.00 | 77.76 | 7 |
| ATOM | 543 | CA | GLU | A | 77 | 19.235 | 75.587 | 22.082 | 1.00 | 76.31 | 6 |
| ATOM | 544 | CB | GLU | A | 77 | 18.611 | 76.604 | 21.133 | 1.00 | 63.33 | 6 |
| ATOM | 545 | CG | GLU | A | 77 | 18.347 | 77.953 | 21.750 | 1.00 | 64.17 | 6 |
| ATOM | 546 | CD | GLU | A | 77 | 17.173 | 78.659 | 21.102 | 1.00 | 64.78 | 6 |
| ATOM | 547 | OE1 | GLU | A | 77 | 16.026 | 78.202 | 21.309 | 1.00 | 64.71 | 8 |
| ATOM | 548 | OE2 | GLU | A | 77 | 17.394 | 79.660 | 20.387 | 1.00 | 65.41 | 8 |
| ATOM | 549 | C | GLU | A | 77 | 18.295 | 74.393 | 22.206 | 1.00 | 75.12 | 6 |
| ATOM | 550 | O | GLU | A | 77 | 17.823 | 74.062 | 23.293 | 1.00 | 75.46 | 8 |
| ATOM | 551 | N | ILE | A | 78 | 18.038 | 73.755 | 21.066 | 1.00 | 32.87 | 7 |
| ATOM | 552 | CA | ILE | A | 78 | 17.176 | 72.583 | 20.982 | 1.00 | 30.59 | 6 |
| ATOM | 553 | CB | ILE | A | 78 | 17.406 | 71.828 | 19.661 | 1.00 | 32.78 | 6 |
| ATOM | 554 | CG2 | ILE | A | 78 | 16.570 | 70.569 | 19.627 | 1.00 | 31.04 | 6 |
| ATOM | 555 | CG1 | ILE | A | 78 | 17.049 | 72.743 | 18.482 | 1.00 | 33.50 | 6 |
| ATOM | 556 | CD1 | ILE | A | 78 | 17.468 | 72.214 | 17.114 | 1.00 | 34.08 | 6 |
| ATOM | 557 | C | ILE | A | 78 | 17.462 | 71.645 | 22.136 | 1.00 | 30.32 | 6 |
| ATOM | 558 | O | ILE | A | 78 | 16.544 | 71.190 | 22.818 | 1.00 | 29.37 | 8 |
| ATOM | 559 | N | ILE | A | 79 | 18.737 | 71.361 | 22.362 | 1.00 | 37.89 | 7 |
| ATOM | 560 | CA | ILE | A | 79 | 19.094 | 70.471 | 23.441 | 1.00 | 39.91 | 6 |
| ATOM | 561 | CB | ILE | A | 79 | 20.512 | 69.954 | 23.281 | 1.00 | 45.66 | 6 |
| ATOM | 562 | CG2 | ILE | A | 79 | 20.957 | 69.240 | 24.534 | 1.00 | 45.36 | 6 |
| ATOM | 563 | CG1 | ILE | A | 79 | 20.561 | 68.981 | 22.113 | 1.00 | 46.47 | 6 |
| ATOM | 564 | CD1 | ILE | A | 79 | 21.890 | 68.301 | 21.967 | 1.00 | 48.56 | 6 |
| ATOM | 565 | C | ILE | A | 79 | 18.951 | 71.160 | 24.784 | 1.00 | 41.42 | 6 |
| ATOM | 566 | O | ILE | A | 79 | 18.754 | 70.499 | 25.810 | 1.00 | 42.07 | 8 |
| ATOM | 567 | N | LEU | A | 80 | 19.050 | 72.488 | 24.786 | 1.00 | 89.16 | 7 |
| ATOM | 568 | CA | LEU | A | 80 | 18.912 | 73.250 | 26.023 | 1.00 | 89.46 | 6 |
| ATOM | 569 | CB | LEU | A | 80 | 19.462 | 74.678 | 25.856 | 1.00 | 35.09 | 6 |
| ATOM | 570 | CG | LEU | A | 80 | 20.982 | 74.838 | 26.063 | 1.00 | 34.34 | 6 |
| ATOM | 571 | CD1 | LEU | A | 80 | 21.423 | 76.281 | 25.845 | 1.00 | 34.07 | 6 |
| ATOM | 572 | CD2 | LEU | A | 80 | 21.331 | 74.392 | 27.482 | 1.00 | 33.85 | 6 |
| ATOM | 573 | C | LEU | A | 80 | 17.447 | 73.279 | 26.425 | 1.00 | 89.37 | 6 |
| ATOM | 574 | O | LEU | A | 80 | 17.120 | 73.370 | 27.609 | 1.00 | 90.96 | 8 |
| ATOM | 575 | N | ASN | A | 81 | 16.569 | 73.189 | 25.428 | 1.00 | 41.89 | 7 |
| ATOM | 576 | CA | ASN | A | 81 | 15.134 | 73.170 | 25.674 | 1.00 | 41.74 | 6 |
| ATOM | 577 | CB | ASN | A | 81 | 14.362 | 73.564 | 24.412 | 1.00 | 69.15 | 6 |
| ATOM | 578 | CG | ASN | A | 81 | 14.578 | 75.019 | 24.024 | 1.00 | 70.45 | 6 |
| ATOM | 579 | OD1 | ASN | A | 81 | 15.661 | 75.406 | 23.595 | 1.00 | 70.24 | 8 |
| ATOM | 580 | ND2 | ASN | A | 81 | 13.542 | 75.833 | 24.180 | 1.00 | 71.82 | 7 |
| ATOM | 581 | C | ASN | A | 81 | 14.731 | 71.766 | 26.125 | 1.00 | 40.96 | 6 |
| ATOM | 582 | O | ASN | A | 81 | 14.149 | 71.601 | 27.200 | 1.00 | 40.05 | 8 |
| ATOM | 583 | N | LEU | A | 82 | 15.053 | 70.755 | 25.316 | 1.00 | 63.42 | 7 |
| ATOM | 584 | CA | LEU | A | 82 | 14.724 | 69.372 | 25.667 | 1.00 | 64.05 | 6 |
| ATOM | 585 | CB | LEU | A | 82 | 15.370 | 68.366 | 24.700 | 1.00 | 58.81 | 6 |
| ATOM | 586 | CG | LEU | A | 82 | 14.932 | 68.301 | 23.231 | 1.00 | 59.30 | 6 |
| ATOM | 587 | CD1 | LEU | A | 82 | 15.628 | 67.112 | 22.568 | 1.00 | 58.70 | 6 |
| ATOM | 588 | CD2 | LEU | A | 82 | 13.411 | 68.159 | 23.123 | 1.00 | 59.58 | 6 |
| ATOM | 589 | C | LEU | A | 82 | 15.200 | 69.062 | 27.074 | 1.00 | 64.25 | 6 |
| ATOM | 590 | O | LEU | A | 82 | 14.629 | 68.233 | 27.766 | 1.00 | 64.54 | 8 |
| ATOM | 591 | N | LYS | A | 83 | 16.255 | 69.730 | 27.504 | 1.00 | 93.29 | 7 |
| ATOM | 592 | CA | LYS | A | 83 | 16.771 | 69.490 | 28.835 | 1.00 | 95.39 | 6 |
| ATOM | 593 | CB | LYS | A | 83 | 17.951 | 70.441 | 29.100 | 1.00 | 57.62 | 6 |
| ATOM | 594 | CG | LYS | A | 83 | 18.613 | 70.272 | 30.464 | 1.00 | 58.61 | 6 |
| ATOM | 595 | CD | LYS | A | 83 | 19.963 | 70.983 | 30.554 | 1.00 | 58.45 | 6 |
| ATOM | 596 | CE | LYS | A | 83 | 21.016 | 70.259 | 29.735 | 1.00 | 58.82 | 6 |
| ATOM | 597 | NZ | LYS | A | 83 | 22.365 | 70.853 | 29.907 | 1.00 | 57.41 | 7 |
| ATOM | 598 | C | LYS | A | 83 | 15.651 | 69.675 | 29.871 | 1.00 | 96.28 | 6 |
| ATOM | 599 | O | LYS | A | 83 | 15.738 | 69.164 | 30.990 | 1.00 | 97.41 | 8 |
| ATOM | 600 | N | GLU | A | 84 | 14.583 | 70.368 | 29.472 | 1.00 | 83.14 | 7 |
| ATOM | 601 | CA | GLU | A | 84 | 13.446 | 70.653 | 30.354 | 1.00 | 83.07 | 6 |
| ATOM | 602 | CB | GLU | A | 84 | 13.032 | 72.108 | 30.185 | 1.00 | 62.64 | 6 |
| ATOM | 603 | CG | GLU | A | 84 | 14.028 | 73.098 | 30.716 | 1.00 | 63.41 | 6 |
| ATOM | 604 | CD | GLU | A | 84 | 13.747 | 74.495 | 30.214 | 1.00 | 65.27 | 6 |
| ATOM | 605 | OE1 | GLU | A | 84 | 12.552 | 74.877 | 30.155 | 1.00 | 66.33 | 8 |
| ATOM | 606 | OE2 | GLU | A | 84 | 14.719 | 75.216 | 29.883 | 1.00 | 66.86 | 8 |
| ATOM | 607 | C | GLU | A | 84 | 12.200 | 69.779 | 30.191 | 1.00 | 82.17 | 6 |
| ATOM | 608 | O | GLU | A | 84 | 11.248 | 69.902 | 30.957 | 1.00 | 82.78 | 8 |
| ATOM | 609 | N | LEU | A | 85 | 12.199 | 68.913 | 29.189 | 1.00 | 32.42 | 7 |
| ATOM | 610 | CA | LEU | A | 85 | 11.065 | 68.033 | 28.929 | 1.00 | 30.61 | 6 |
| ATOM | 611 | CB | LEU | A | 85 | 11.322 | 67.244 | 27.649 | 1.00 | 29.83 | 6 |
| ATOM | 612 | CG | LEU | A | 85 | 10.253 | 66.272 | 27.182 | 1.00 | 28.15 | 6 |
| ATOM | 613 | CD1 | LEU | A | 85 | 9.219 | 67.034 | 26.338 | 1.00 | 27.30 | 6 |
| ATOM | 614 | CD2 | LEU | A | 85 | 10.921 | 65.138 | 26.386 | 1.00 | 27.81 | 6 |
| ATOM | 615 | C | LEU | A | 85 | 10.857 | 67.065 | 30.086 | 1.00 | 30.06 | 6 |
| ATOM | 616 | O | LEU | A | 85 | 11.779 | 66.380 | 30.499 | 1.00 | 28.59 | 8 |
| ATOM | 617 | N | VAL | A | 86 | 9.645 | 67.007 | 30.617 | 1.00 | 42.85 | 7 |
| ATOM | 618 | CA | VAL | A | 86 | 9.384 | 66.093 | 31.716 | 1.00 | 43.95 | 6 |
| ATOM | 619 | CB | VAL | A | 86 | 8.760 | 66.804 | 32.932 | 1.00 | 39.00 | 6 |
| ATOM | 620 | CG1 | VAL | A | 86 | 7.557 | 67.620 | 32.527 | 1.00 | 38.89 | 6 |
| ATOM | 621 | CG2 | VAL | A | 86 | 8.368 | 65.766 | 33.960 | 1.00 | 40.01 | 6 |
| ATOM | 622 | C | VAL | A | 86 | 8.462 | 64.972 | 31.298 | 1.00 | 43.36 | 6 |
| ATOM | 623 | O | VAL | A | 86 | 7.309 | 65.215 | 30.938 | 1.00 | 43.19 | 8 |
| ATOM | 624 | N | VAL | A | 87 | 8.966 | 63.743 | 31.351 | 1.00 | 21.15 | 7 |
| ATOM | 625 | CA | VAL | A | 87 | 8.154 | 62.603 | 30.958 | 1.00 | 23.38 | 6 |
| ATOM | 626 | CB | VAL | A | 87 | 8.903 | 61.625 | 30.004 | 1.00 | 39.94 | 6 |
| ATOM | 627 | CG1 | VAL | A | 87 | 9.272 | 62.330 | 28.711 | 1.00 | 40.80 | 6 |
| ATOM | 628 | CG2 | VAL | A | 87 | 10.139 | 61.075 | 30.676 | 1.00 | 42.12 | 6 |
| ATOM | 629 | C | VAL | A | 87 | 7.702 | 61.822 | 32.157 | 1.00 | 24.21 | 6 |
| ATOM | 630 | O | VAL | A | 87 | 8.396 | 61.768 | 33.169 | 1.00 | 23.51 | 8 |
| ATOM | 631 | N | ARG | A | 88 | 6.512 | 61.243 | 32.026 | 1.00 | 76.51 | 7 |
| ATOM | 632 | CA | ARG | A | 88 | 5.903 | 60.411 | 33.049 | 1.00 | 80.48 | 6 |
| ATOM | 633 | CB | ARG | A | 88 | 4.408 | 60.688 | 33.132 | 1.00 | 63.66 | 6 |
| ATOM | 634 | CG | ARG | A | 88 | 3.694 | 59.846 | 34.164 | 1.00 | 64.46 | 6 |
| ATOM | 635 | CD | ARG | A | 88 | 2.414 | 60.503 | 34.634 | 1.00 | 65.19 | 6 |
| ATOM | 636 | NE | ARG | A | 88 | 1.763 | 59.729 | 35.683 | 1.00 | 66.10 | 7 |
| ATOM | 637 | CZ | ARG | A | 88 | 0.793 | 60.196 | 36.463 | 1.00 | 68.02 | 6 |
| ATOM | 638 | NH1 | ARG | A | 88 | 0.259 | 59.417 | 37.392 | 1.00 | 69.40 | 7 |
| ATOM | 639 | NH2 | ARG | A | 88 | 0.356 | 61.443 | 36.322 | 1.00 | 68.70 | 7 |
| ATOM | 640 | C | ARG | A | 88 | 6.142 | 59.014 | 32.517 | 1.00 | 82.14 | 6 |
| ATOM | 641 | O | ARG | A | 88 | 6.387 | 58.861 | 31.326 | 1.00 | 83.19 | 8 |
| ATOM | 642 | N | PHE | A | 89 | 6.073 | 57.991 | 33.362 | 1.00 | 58.23 | 7 |
| ATOM | 643 | CA | PHE | A | 89 | 6.326 | 56.644 | 32.862 | 1.00 | 61.38 | 6 |
| ATOM | 644 | CB | PHE | A | 89 | 7.625 | 56.125 | 33.483 | 1.00 | 54.32 | 6 |
| ATOM | 645 | CG | PHE | A | 89 | 8.849 | 56.458 | 32.668 | 1.00 | 52.53 | 6 |
| ATOM | 646 | CD1 | PHE | A | 89 | 8.843 | 57.539 | 31.788 | 1.00 | 51.76 | 6 |
| ATOM | 647 | CD2 | PHE | A | 89 | 9.994 | 55.672 | 32.752 | 1.00 | 51.86 | 6 |
| ATOM | 648 | CE1 | PHE | A | 89 | 9.953 | 57.831 | 31.009 | 1.00 | 50.89 | 6 |
| ATOM | 649 | CE2 | PHE | A | 89 | 11.114 | 55.956 | 31.976 | 1.00 | 50.55 | 6 |
| ATOM | 650 | CZ | PHE | A | 89 | 11.091 | 57.037 | 31.097 | 1.00 | 50.23 | 6 |
| ATOM | 651 | C | PHE | A | 89 | 5.210 | 55.595 | 32.952 | 1.00 | 63.31 | 6 |
| ATOM | 652 | O | PHE | A | 89 | 5.355 | 54.491 | 32.432 | 1.00 | 64.67 | 8 |
| ATOM | 653 | N | LEU | A | 90 | 4.097 | 55.938 | 33.591 | 1.00 | 77.73 | 7 |
| ATOM | 654 | CA | LEU | A | 90 | 2.952 | 55.033 | 33.721 | 1.00 | 80.08 | 6 |
| ATOM | 655 | CB | LEU | A | 90 | 2.105 | 55.065 | 32.447 | 1.00 | 30.07 | 6 |
| ATOM | 656 | CG | LEU | A | 90 | 1.734 | 56.412 | 31.834 | 1.00 | 29.09 | 6 |
| ATOM | 657 | CD1 | LEU | A | 90 | 0.710 | 56.165 | 30.757 | 1.00 | 28.59 | 6 |
| ATOM | 658 | CD2 | LEU | A | 90 | 1.149 | 57.351 | 32.870 | 1.00 | 29.27 | 6 |
| ATOM | 659 | C | LEU | A | 90 | 3.250 | 53.569 | 34.042 | 1.00 | 81.65 | 6 |
| ATOM | 660 | O | LEU | A | 90 | 2.348 | 52.738 | 33.956 | 1.00 | 82.47 | 8 |
| ATOM | 661 | N | ASP | A | 91 | 4.492 | 53.244 | 34.396 | 1.00 | 108.42 | 7 |
| ATOM | 662 | CA | ASP | A | 91 | 4.856 | 51.862 | 34.719 | 1.00 | 111.01 | 6 |
| ATOM | 663 | CB | ASP | A | 91 | 4.874 | 50.999 | 33.471 | 1.00 | 109.60 | 6 |
| ATOM | 664 | CG | ASP | A | 91 | 6.246 | 50.931 | 32.855 | 1.00 | 111.18 | 6 |
| ATOM | 665 | OD1 | ASP | A | 91 | 6.576 | 51.826 | 32.056 | 1.00 | 111.61 | 8 |
| ATOM | 666 | OD2 | ASP | A | 91 | 7.003 | 49.995 | 33.190 | 1.00 | 112.73 | 8 |
| ATOM | 667 | C | ASP | A | 91 | 6.240 | 51.764 | 35.345 | 1.00 | 111.70 | 6 |
| ATOM | 668 | O | ASP | A | 91 | 6.993 | 52.736 | 35.366 | 1.00 | 112.36 | 8 |
| ATOM | 669 | N | PRO | A | 92 | 6.596 | 50.577 | 35.856 | 1.00 | 72.17 | 7 |
| ATOM | 670 | CD | PRO | A | 92 | 5.630 | 49.603 | 36.396 | 1.00 | 138.10 | 6 |
| ATOM | 671 | CA | PRO | A | 92 | 7.910 | 50.389 | 36.475 | 1.00 | 72.33 | 6 |
| ATOM | 672 | CB | PRO | A | 92 | 7.560 | 49.702 | 37.781 | 1.00 | 138.16 | 6 |
| ATOM | 673 | CG | PRO | A | 92 | 6.484 | 48.769 | 37.341 | 1.00 | 138.73 | 6 |
| ATOM | 674 | C | PRO | A | 92 | 8.902 | 49.564 | 35.655 | 1.00 | 71.96 | 6 |
| ATOM | 675 | O | PRO | A | 92 | 8.517 | 48.719 | 34.844 | 1.00 | 71.56 | 8 |
| ATOM | 676 | N | ALA | A | 93 | 10.186 | 49.841 | 35.874 | 1.00 | 162.67 | 7 |
| ATOM | 677 | CA | ALA | A | 93 | 11.286 | 49.132 | 35.225 | 1.00 | 162.90 | 6 |
| ATOM | 678 | CB | ALA | A | 93 | 11.106 | 47.636 | 35.429 | 1.00 | 114.11 | 6 |
| ATOM | 679 | C | ALA | A | 93 | 11.551 | 49.407 | 33.747 | 1.00 | 162.76 | 6 |
| ATOM | 680 | O | ALA | A | 93 | 11.126 | 48.620 | 32.900 | 1.00 | 162.55 | 8 |
| ATOM | 681 | N | TRP | A | 94 | 12.270 | 50.493 | 33.442 | 1.00 | 96.59 | 7 |
| ATOM | 682 | CA | TRP | A | 94 | 12.607 | 50.826 | 32.055 | 1.00 | 95.32 | 6 |
| ATOM | 683 | CB | TRP | A | 94 | 11.431 | 50.488 | 31.137 | 1.00 | 107.02 | 6 |
| ATOM | 684 | CG | TRP | A | 94 | 10.337 | 51.499 | 31.193 | 1.00 | 108.85 | 6 |
| ATOM | 685 | CD2 | TRP | A | 94 | 9.655 | 52.086 | 30.083 | 1.00 | 110.00 | 6 |
| ATOM | 686 | CE2 | TRP | A | 94 | 8.717 | 52.997 | 30.607 | 1.00 | 111.39 | 6 |
| ATOM | 687 | CE3 | TRP | A | 94 | 9.745 | 51.932 | 28.695 | 1.00 | 110.71 | 6 |
| ATOM | 688 | CD1 | TRP | A | 94 | 9.800 | 52.057 | 32.312 | 1.00 | 110.15 | 6 |
| ATOM | 689 | NE1 | TRP | A | 94 | 8.829 | 52.959 | 31.971 | 1.00 | 111.87 | 7 |
| ATOM | 690 | CZ2 | TRP | A | 94 | 7.871 | 53.756 | 29.792 | 1.00 | 112.08 | 6 |
| ATOM | 691 | CZ3 | TRP | A | 94 | 8.902 | 52.687 | 27.885 | 1.00 | 112.05 | 6 |
| ATOM | 692 | CH2 | TRP | A | 94 | 7.979 | 53.587 | 28.438 | 1.00 | 112.66 | 6 |
| ATOM | 693 | C | TRP | A | 94 | 13.012 | 52.272 | 31.763 | 1.00 | 92.85 | 6 |
| ATOM | 694 | O | TRP | A | 94 | 12.934 | 53.149 | 32.623 | 1.00 | 92.99 | 8 |
| ATOM | 695 | N | ARG | A | 95 | 13.433 | 52.482 | 30.514 | 1.00 | 77.36 | 7 |
| ATOM | 696 | CA | ARG | A | 95 | 13.819 | 53.780 | 29.951 | 1.00 | 73.49 | 6 |
| ATOM | 697 | CB | ARG | A | 95 | 15.327 | 53.988 | 30.002 | 1.00 | 89.27 | 6 |
| ATOM | 698 | CG | ARG | A | 95 | 16.061 | 53.578 | 28.730 | 1.00 | 91.22 | 6 |
| ATOM | 699 | CD | ARG | A | 95 | 16.014 | 52.068 | 28.466 | 1.00 | 92.63 | 6 |
| ATOM | 700 | NE | ARG | A | 95 | 16.719 | 51.720 | 27.231 | 1.00 | 94.64 | 7 |
| ATOM | 701 | CZ | ARG | A | 95 | 16.777 | 50.500 | 26.706 | 1.00 | 96.74 | 6 |
| ATOM | 702 | NH1 | ARG | A | 95 | 16.164 | 49.483 | 27.305 | 1.00 | 97.64 | 7 |
| ATOM | 703 | NH2 | ARG | A | 95 | 17.452 | 50.299 | 25.579 | 1.00 | 98.03 | 7 |
| ATOM | 704 | C | ARG | A | 95 | 13.379 | 53.654 | 28.493 | 1.00 | 69.86 | 6 |
| ATOM | 705 | O | ARG | A | 95 | 13.380 | 52.562 | 27.946 | 1.00 | 70.69 | 8 |
| ATOM | 706 | N | THR | A | 96 | 13.012 | 54.745 | 27.846 | 1.00 | 30.81 | 7 |
| ATOM | 707 | CA | THR | A | 96 | 12.552 | 54.628 | 26.469 | 1.00 | 26.69 | 6 |
| ATOM | 708 | CB | THR | A | 96 | 10.978 | 54.571 | 26.415 | 1.00 | 13.87 | 6 |
| ATOM | 709 | OG1 | THR | A | 96 | 10.537 | 54.617 | 25.053 | 1.00 | 13.87 | 8 |
| ATOM | 710 | CG2 | THR | A | 96 | 10.353 | 55.715 | 27.192 | 1.00 | 13.87 | 6 |
| ATOM | 711 | C | THR | A | 96 | 13.093 | 55.728 | 25.560 | 1.00 | 24.46 | 6 |
| ATOM | 712 | O | THR | A | 96 | 13.990 | 56.467 | 25.967 | 1.00 | 23.10 | 8 |
| ATOM | 713 | N | THR | A | 97 | 12.554 | 55.839 | 24.342 | 1.00 | 70.32 | 7 |
| ATOM | 714 | CA | THR | A | 97 | 13.029 | 56.845 | 23.400 | 1.00 | 68.74 | 6 |
| ATOM | 715 | CB | THR | A | 97 | 14.070 | 56.273 | 22.467 | 1.00 | 66.34 | 6 |
| ATOM | 716 | OG1 | THR | A | 97 | 14.815 | 55.258 | 23.147 | 1.00 | 67.45 | 8 |
| ATOM | 717 | CG2 | THR | A | 97 | 15.001 | 57.381 | 21.986 | 1.00 | 65.31 | 6 |
| ATOM | 718 | C | THR | A | 97 | 11.998 | 57.494 | 22.497 | 1.00 | 67.24 | 6 |
| ATOM | 719 | O | THR | A | 97 | 11.613 | 56.927 | 21.478 | 1.00 | 68.97 | 8 |
| ATOM | 720 | N | LEU | A | 98 | 11.589 | 58.704 | 22.856 | 1.00 | 38.90 | 7 |
| ATOM | 721 | CA | LEU | A | 98 | 10.628 | 59.478 | 22.079 | 1.00 | 36.55 | 6 |
| ATOM | 722 | CB | LEU | A | 98 | 10.416 | 60.843 | 22.750 | 1.00 | 32.16 | 6 |
| ATOM | 723 | CG | LEU | A | 98 | 9.636 | 60.817 | 24.077 | 1.00 | 31.78 | 6 |
| ATOM | 724 | CD1 | LEU | A | 98 | 8.132 | 60.750 | 23.797 | 1.00 | 31.19 | 6 |
| ATOM | 725 | CD2 | LEU | A | 98 | 10.073 | 59.627 | 24.931 | 1.00 | 30.42 | 6 |
| ATOM | 726 | C | LEU | A | 98 | 11.131 | 59.647 | 20.638 | 1.00 | 35.43 | 6 |
| ATOM | 727 | O | LEU | A | 98 | 12.309 | 59.422 | 20.348 | 1.00 | 37.05 | 8 |
| ATOM | 728 | N | ILE | A | 99 | 10.220 | 60.023 | 19.746 | 1.00 | 13.87 | 7 |
| ATOM | 729 | CA | ILE | A | 99 | 10.493 | 60.208 | 18.322 | 1.00 | 13.87 | 6 |
| ATOM | 730 | CB | ILE | A | 99 | 9.951 | 59.037 | 17.487 | 1.00 | 28.58 | 6 |
| ATOM | 731 | CG2 | ILE | A | 99 | 10.336 | 59.220 | 16.058 | 1.00 | 28.83 | 6 |
| ATOM | 732 | CG1 | ILE | A | 99 | 10.455 | 57.698 | 18.045 | 1.00 | 29.22 | 6 |
| ATOM | 733 | CD1 | ILE | A | 99 | 11.967 | 57.497 | 18.012 | 1.00 | 29.54 | 6 |
| ATOM | 734 | C | ILE | A | 99 | 9.749 | 61.451 | 17.892 | 1.00 | 13.87 | 6 |
| ATOM | 735 | O | ILE | A | 99 | 8.802 | 61.856 | 18.546 | 1.00 | 13.87 | 8 |
| ATOM | 736 | N | LEU | A | 100 | 10.149 | 62.063 | 16.793 | 1.00 | 15.12 | 7 |
| ATOM | 737 | CA | LEU | A | 100 | 9.451 | 63.261 | 16.368 | 1.00 | 18.83 | 6 |
| ATOM | 738 | CB | LEU | A | 100 | 9.689 | 64.400 | 17.362 | 1.00 | 24.33 | 6 |
| ATOM | 739 | CG | LEU | A | 100 | 9.854 | 65.811 | 16.787 | 1.00 | 24.43 | 6 |
| ATOM | 740 | CD1 | LEU | A | 100 | 8.642 | 66.171 | 16.002 | 1.00 | 26.73 | 6 |
| ATOM | 741 | CD2 | LEU | A | 100 | 10.055 | 66.820 | 17.892 | 1.00 | 23.82 | 6 |
| ATOM | 742 | C | LEU | A | 100 | 9.858 | 63.709 | 14.994 | 1.00 | 19.65 | 6 |
| ATOM | 743 | O | LEU | A | 100 | 11.032 | 63.946 | 14.734 | 1.00 | 19.44 | 8 |
| ATOM | 744 | N | ARG | A | 101 | 8.858 | 63.847 | 14.133 | 1.00 | 55.57 | 7 |
| ATOM | 745 | CA | ARG | A | 101 | 9.036 | 64.286 | 12.757 | 1.00 | 58.59 | 6 |
| ATOM | 746 | CB | ARG | A | 101 | 8.513 | 63.191 | 11.815 | 1.00 | 132.06 | 6 |
| ATOM | 747 | CG | ARG | A | 101 | 9.239 | 63.046 | 10.478 | 1.00 | 135.60 | 6 |
| ATOM | 748 | CD | ARG | A | 101 | 8.651 | 61.883 | 9.672 | 1.00 | 137.39 | 6 |
| ATOM | 749 | NE | ARG | A | 101 | 9.392 | 61.598 | 8.443 | 1.00 | 139.28 | 7 |
| ATOM | 750 | CZ | ARG | A | 101 | 9.039 | 60.669 | 7.556 | 1.00 | 140.18 | 6 |
| ATOM | 751 | NH1 | ARG | A | 101 | 7.953 | 59.931 | 7.756 | 1.00 | 139.80 | 7 |
| ATOM | 752 | NH2 | ARG | A | 101 | 9.777 | 60.467 | 6.472 | 1.00 | 140.56 | 7 |
| ATOM | 753 | C | ARG | A | 101 | 8.192 | 65.564 | 12.623 | 1.00 | 58.89 | 6 |
| ATOM | 754 | O | ARG | A | 101 | 7.064 | 65.626 | 13.129 | 1.00 | 59.23 | 8 |
| ATOM | 755 | N | ALA | A | 102 | 8.720 | 66.587 | 11.961 | 1.00 | 35.89 | 7 |
| ATOM | 756 | CA | ALA | A | 102 | 7.947 | 67.821 | 11.811 | 1.00 | 38.61 | 6 |
| ATOM | 757 | CB | ALA | A | 102 | 8.293 | 68.793 | 12.932 | 1.00 | 67.58 | 6 |
| ATOM | 758 | C | ALA | A | 102 | 8.137 | 68.493 | 10.451 | 1.00 | 38.78 | 6 |
| ATOM | 759 | O | ALA | A | 102 | 9.262 | 68.718 | 10.003 | 1.00 | 38.84 | 8 |
| ATOM | 760 | N | GLU | A | 103 | 7.030 | 68.835 | 9.806 | 1.00 | 34.50 | 7 |
| ATOM | 761 | CA | GLU | A | 103 | 7.088 | 69.445 | 8.488 | 1.00 | 36.32 | 6 |
| ATOM | 762 | CB | GLU | A | 103 | 5.869 | 69.029 | 7.673 | 1.00 | 66.19 | 6 |
| ATOM | 763 | CG | GLU | A | 103 | 5.987 | 67.636 | 7.113 | 1.00 | 66.55 | 6 |
| ATOM | 764 | CD | GLU | A | 103 | 7.340 | 67.422 | 6.499 | 1.00 | 65.97 | 6 |
| ATOM | 765 | OE1 | GLU | A | 103 | 7.770 | 68.300 | 5.719 | 1.00 | 64.19 | 8 |
| ATOM | 766 | OE2 | GLU | A | 103 | 7.973 | 66.387 | 6.802 | 1.00 | 66.71 | 8 |
| ATOM | 767 | C | GLU | A | 103 | 7.221 | 70.952 | 8.435 | 1.00 | 38.51 | 6 |
| ATOM | 768 | O | GLU | A | 103 | 7.263 | 71.638 | 9.456 | 1.00 | 38.40 | 8 |
| ATOM | 769 | N | GLY | A | 104 | 7.280 | 71.444 | 7.202 | 1.00 | 82.92 | 7 |
| ATOM | 770 | CA | GLY | A | 104 | 7.412 | 72.859 | 6.906 | 1.00 | 86.20 | 6 |
| ATOM | 771 | C | GLY | A | 104 | 7.259 | 73.885 | 8.006 | 1.00 | 87.20 | 6 |
| ATOM | 772 | O | GLY | A | 104 | 8.069 | 73.938 | 8.927 | 1.00 | 87.75 | 8 |
| ATOM | 773 | N | PRO | A | 105 | 6.220 | 74.729 | 7.925 | 1.00 | 121.54 | 7 |
| ATOM | 774 | CD | PRO | A | 105 | 5.240 | 74.798 | 6.823 | 1.00 | 82.59 | 6 |
| ATOM | 775 | CA | PRO | A | 105 | 5.956 | 75.775 | 8.911 | 1.00 | 122.33 | 6 |
| ATOM | 776 | CB | PRO | A | 105 | 5.165 | 76.789 | 8.101 | 1.00 | 82.71 | 6 |
| ATOM | 777 | CG | PRO | A | 105 | 4.280 | 75.890 | 7.292 | 1.00 | 82.69 | 6 |
| ATOM | 778 | C | PRO | A | 105 | 5.187 | 75.310 | 10.143 | 1.00 | 121.71 | 6 |
| ATOM | 779 | O | PRO | A | 105 | 4.319 | 74.442 | 10.050 | 1.00 | 121.21 | 8 |
| ATOM | 780 | N | LYS | A | 106 | 5.525 | 75.908 | 11.284 | 1.00 | 81.15 | 7 |
| ATOM | 781 | CA | LYS | A | 106 | 4.887 | 75.646 | 12.572 | 1.00 | 81.76 | 6 |
| ATOM | 782 | CB | LYS | A | 106 | 4.137 | 74.322 | 12.567 | 1.00 | 80.86 | 6 |
| ATOM | 783 | CG | LYS | A | 106 | 5.010 | 73.105 | 12.524 | 1.00 | 80.61 | 6 |
| ATOM | 784 | CD | LYS | A | 106 | 4.113 | 71.913 | 12.641 | 1.00 | 82.64 | 6 |
| ATOM | 785 | CE | LYS | A | 106 | 4.884 | 70.632 | 12.761 | 1.00 | 84.29 | 6 |
| ATOM | 786 | NZ | LYS | A | 106 | 3.925 | 69.490 | 12.835 | 1.00 | 86.22 | 7 |
| ATOM | 787 | C | LYS | A | 106 | 5.885 | 75.632 | 13.711 | 1.00 | 81.50 | 6 |
| ATOM | 788 | O | LYS | A | 106 | 7.014 | 76.092 | 13.565 | 1.00 | 81.66 | 8 |
| ATOM | 789 | N | GLU | A | 107 | 5.447 | 75.108 | 14.849 | 1.00 | 61.87 | 7 |
| ATOM | 790 | CA | GLU | A | 107 | 6.283 | 75.001 | 16.035 | 1.00 | 62.76 | 6 |
| ATOM | 791 | CB | GLU | A | 107 | 5.757 | 75.913 | 17.136 | 1.00 | 63.05 | 6 |
| ATOM | 792 | CG | GLU | A | 107 | 5.992 | 77.370 | 16.866 | 1.00 | 62.98 | 6 |
| ATOM | 793 | CD | GLU | A | 107 | 5.557 | 78.241 | 18.015 | 1.00 | 63.40 | 6 |
| ATOM | 794 | OE1 | GLU | A | 107 | 5.837 | 77.874 | 19.181 | 1.00 | 62.44 | 8 |
| ATOM | 795 | OE2 | GLU | A | 107 | 4.941 | 79.295 | 17.743 | 1.00 | 64.68 | 8 |
| ATOM | 796 | C | GLU | A | 107 | 6.275 | 73.563 | 16.524 | 1.00 | 62.81 | 6 |
| ATOM | 797 | O | GLU | A | 107 | 5.216 | 72.949 | 16.628 | 1.00 | 64.05 | 8 |
| ATOM | 798 | N | VAL | A | 108 | 7.445 | 73.015 | 16.828 | 1.00 | 92.35 | 7 |
| ATOM | 799 | CA | VAL | A | 108 | 7.489 | 71.641 | 17.298 | 1.00 | 90.77 | 6 |
| ATOM | 800 | CB | VAL | A | 108 | 8.786 | 70.926 | 16.886 | 1.00 | 24.79 | 6 |
| ATOM | 801 | CG1 | VAL | A | 108 | 8.524 | 69.456 | 16.759 | 1.00 | 22.72 | 6 |
| ATOM | 802 | CG2 | VAL | A | 108 | 9.305 | 71.473 | 15.599 | 1.00 | 23.64 | 6 |
| ATOM | 803 | C | VAL | A | 108 | 7.373 | 71.569 | 18.813 | 1.00 | 90.99 | 6 |
| ATOM | 804 | O | VAL | A | 108 | 8.067 | 70.785 | 19.448 | 1.00 | 91.87 | 8 |
| ATOM | 805 | N | ARG | A | 109 | 6.504 | 72.389 | 19.394 | 1.00 | 16.19 | 7 |
| ATOM | 806 | CA | ARG | A | 109 | 6.318 | 72.367 | 20.836 | 1.00 | 17.08 | 6 |
| ATOM | 807 | CB | ARG | A | 109 | 5.075 | 73.192 | 21.230 | 1.00 | 79.46 | 6 |
| ATOM | 808 | CG | ARG | A | 109 | 3.702 | 72.618 | 20.870 | 1.00 | 82.11 | 6 |
| ATOM | 809 | CD | ARG | A | 109 | 3.528 | 72.443 | 19.373 | 1.00 | 82.88 | 6 |
| ATOM | 810 | NE | ARG | A | 109 | 2.130 | 72.272 | 18.982 | 1.00 | 82.78 | 7 |
| ATOM | 811 | CZ | ARG | A | 109 | 1.738 | 71.912 | 17.762 | 1.00 | 83.25 | 6 |
| ATOM | 812 | NH1 | ARG | A | 109 | 2.636 | 71.675 | 16.816 | 1.00 | 82.30 | 7 |
| ATOM | 813 | NH2 | ARG | A | 109 | 0.448 | 71.812 | 17.477 | 1.00 | 83.70 | 7 |
| ATOM | 814 | C | ARG | A | 109 | 6.211 | 70.916 | 21.329 | 1.00 | 16.63 | 6 |
| ATOM | 815 | O | ARG | A | 109 | 5.687 | 70.052 | 20.636 | 1.00 | 16.88 | 8 |
| ATOM | 816 | N | ALA | A | 110 | 6.733 | 70.653 | 22.520 | 1.00 | 43.26 | 7 |
| ATOM | 817 | CA | ALA | A | 110 | 6.740 | 69.314 | 23.119 | 1.00 | 44.15 | 6 |
| ATOM | 818 | CB | ALA | A | 110 | 6.592 | 69.434 | 24.630 | 1.00 | 62.64 | 6 |
| ATOM | 819 | C | ALA | A | 110 | 5.731 | 68.300 | 22.582 | 1.00 | 44.52 | 6 |
| ATOM | 820 | O | ALA | A | 110 | 6.111 | 67.264 | 22.031 | 1.00 | 43.73 | 8 |
| ATOM | 821 | N | VAL | A | 111 | 4.450 | 68.589 | 22.757 | 1.00 | 59.09 | 7 |
| ATOM | 822 | CA | VAL | A | 111 | 3.391 | 67.687 | 22.302 | 1.00 | 60.52 | 6 |
| ATOM | 823 | CB | VAL | A | 111 | 2.128 | 68.466 | 21.894 | 1.00 | 208.87 | 6 |
| ATOM | 824 | CG1 | VAL | A | 111 | 1.013 | 67.493 | 21.525 | 1.00 | 208.87 | 6 |
| ATOM | 825 | CG2 | VAL | A | 111 | 1.698 | 69.385 | 23.021 | 1.00 | 208.87 | 6 |
| ATOM | 826 | C | VAL | A | 111 | 3.792 | 66.821 | 21.119 | 1.00 | 58.77 | 6 |
| ATOM | 827 | O | VAL | A | 111 | 3.854 | 65.596 | 21.229 | 1.00 | 58.31 | 8 |
| ATOM | 828 | N | ASP | A | 112 | 4.046 | 67.490 | 19.996 | 1.00 | 43.13 | 7 |
| ATOM | 829 | CA | ASP | A | 112 | 4.447 | 66.875 | 18.733 | 1.00 | 43.00 | 6 |
| ATOM | 830 | CB | ASP | A | 112 | 5.244 | 67.892 | 17.922 | 1.00 | 56.89 | 6 |
| ATOM | 831 | CG | ASP | A | 112 | 4.385 | 69.026 | 17.420 | 1.00 | 56.48 | 6 |
| ATOM | 832 | OD1 | ASP | A | 112 | 3.586 | 68.794 | 16.497 | 1.00 | 55.87 | 8 |
| ATOM | 833 | OD2 | ASP | A | 112 | 4.496 | 70.145 | 17.948 | 1.00 | 56.33 | 8 |
| ATOM | 834 | C | ASP | A | 112 | 5.225 | 65.556 | 18.789 | 1.00 | 42.03 | 6 |
| ATOM | 835 | O | ASP | A | 112 | 5.499 | 64.949 | 17.753 | 1.00 | 41.37 | 8 |
| ATOM | 836 | N | PHE | A | 113 | 5.596 | 65.113 | 19.983 | 1.00 | 66.40 | 7 |
| ATOM | 837 | CA | PHE | A | 113 | 6.326 | 63.861 | 20.106 | 1.00 | 67.18 | 6 |
| ATOM | 838 | CB | PHE | A | 113 | 6.863 | 63.705 | 21.527 | 1.00 | 48.77 | 6 |
| ATOM | 839 | CG | PHE | A | 113 | 8.243 | 64.271 | 21.710 | 1.00 | 45.55 | 6 |
| ATOM | 840 | CD1 | PHE | A | 113 | 8.769 | 65.166 | 20.786 | 1.00 | 43.11 | 6 |
| ATOM | 841 | CD2 | PHE | A | 113 | 9.018 | 63.901 | 22.798 | 1.00 | 44.09 | 6 |
| ATOM | 842 | CE1 | PHE | A | 113 | 10.036 | 65.672 | 20.940 | 1.00 | 42.90 | 6 |
| ATOM | 843 | CE2 | PHE | A | 113 | 10.285 | 64.405 | 22.959 | 1.00 | 43.06 | 6 |
| ATOM | 844 | CZ | PHE | A | 113 | 10.797 | 65.294 | 22.027 | 1.00 | 43.40 | 6 |
| ATOM | 845 | C | PHE | A | 113 | 5.447 | 62.681 | 19.734 | 1.00 | 67.93 | 6 |
| ATOM | 846 | O | PHE | A | 113 | 4.225 | 62.817 | 19.655 | 1.00 | 68.13 | 8 |
| ATOM | 847 | N | THR | A | 114 | 6.077 | 61.532 | 19.500 | 1.00 | 87.75 | 7 |
| ATOM | 848 | CA | THR | A | 114 | 5.366 | 60.317 | 19.122 | 1.00 | 87.95 | 6 |
| ATOM | 849 | CB | THR | A | 114 | 6.351 | 59.177 | 18.778 | 1.00 | 77.17 | 6 |
| ATOM | 850 | OG1 | THR | A | 114 | 5.628 | 58.046 | 18.287 | 1.00 | 75.93 | 8 |
| ATOM | 851 | CG2 | THR | A | 114 | 7.132 | 58.753 | 20.007 | 1.00 | 78.13 | 6 |
| ATOM | 852 | C | THR | A | 114 | 4.433 | 59.833 | 20.226 | 1.00 | 88.39 | 6 |
| ATOM | 853 | O | THR | A | 114 | 4.879 | 59.372 | 21.278 | 1.00 | 88.57 | 8 |
| ATOM | 854 | N | PRO | A | 115 | 3.115 | 59.943 | 20.005 | 1.00 | 98.73 | 7 |
| ATOM | 855 | CD | PRO | A | 115 | 2.382 | 60.327 | 18.785 | 1.00 | 90.85 | 6 |
| ATOM | 856 | CA | PRO | A | 115 | 2.203 | 59.480 | 21.053 | 1.00 | 99.15 | 6 |
| ATOM | 857 | CB | PRO | A | 115 | 0.824 | 59.750 | 20.445 | 1.00 | 90.46 | 6 |
| ATOM | 858 | CG | PRO | A | 115 | 1.071 | 59.620 | 18.964 | 1.00 | 91.10 | 6 |
| ATOM | 859 | C | PRO | A | 115 | 2.491 | 58.003 | 21.267 | 1.00 | 98.39 | 6 |
| ATOM | 860 | O | PRO | A | 115 | 2.155 | 57.190 | 20.415 | 1.00 | 98.70 | 8 |
| ATOM | 861 | N | SER | A | 116 | 3.117 | 57.640 | 22.383 | 1.00 | 43.20 | 7 |
| ATOM | 862 | CA | SER | A | 116 | 3.438 | 56.228 | 22.566 | 1.00 | 43.88 | 6 |
| ATOM | 863 | CB | SER | A | 116 | 4.600 | 55.863 | 21.632 | 1.00 | 127.33 | 6 |
| ATOM | 864 | OG | SER | A | 116 | 5.700 | 56.740 | 21.807 | 1.00 | 127.37 | 8 |
| ATOM | 865 | C | SER | A | 116 | 3.724 | 55.633 | 23.955 | 1.00 | 44.19 | 6 |
| ATOM | 866 | O | SER | A | 116 | 4.506 | 56.168 | 24.747 | 1.00 | 43.78 | 8 |
| ATOM | 867 | N | ALA | A | 117 | 3.087 | 54.490 | 24.210 | 1.00 | 137.91 | 7 |
| ATOM | 868 | CA | ALA | A | 117 | 3.233 | 53.735 | 25.450 | 1.00 | 139.42 | 6 |
| ATOM | 869 | CB | ALA | A | 117 | 4.570 | 53.008 | 25.448 | 1.00 | 13.87 | 6 |
| ATOM | 870 | C | ALA | A | 117 | 3.081 | 54.534 | 26.740 | 1.00 | 139.97 | 6 |
| ATOM | 871 | O | ALA | A | 117 | 2.550 | 55.642 | 26.745 | 1.00 | 139.94 | 8 |
| ATOM | 872 | N | ASP | A | 118 | 3.551 | 53.941 | 27.834 | 1.00 | 85.71 | 7 |
| ATOM | 873 | CA | ASP | A | 118 | 3.482 | 54.546 | 29.160 | 1.00 | 87.10 | 6 |
| ATOM | 874 | CB | ASP | A | 118 | 3.999 | 53.559 | 30.207 | 1.00 | 196.72 | 6 |
| ATOM | 875 | CG | ASP | A | 118 | 3.155 | 52.311 | 30.302 | 1.00 | 198.95 | 6 |
| ATOM | 876 | OD1 | ASP | A | 118 | 1.950 | 52.431 | 30.601 | 1.00 | 200.67 | 8 |
| ATOM | 877 | OD2 | ASP | A | 118 | 3.697 | 51.207 | 30.083 | 1.00 | 199.32 | 8 |
| ATOM | 878 | C | ASP | A | 118 | 4.304 | 55.822 | 29.238 | 1.00 | 86.97 | 6 |
| ATOM | 879 | O | ASP | A | 118 | 5.182 | 55.943 | 30.087 | 1.00 | 87.04 | 8 |
| ATOM | 880 | N | VAL | A | 119 | 4.007 | 56.779 | 28.368 | 1.00 | 144.02 | 7 |
| ATOM | 881 | CA | VAL | A | 119 | 4.756 | 58.026 | 28.339 | 1.00 | 143.57 | 6 |
| ATOM | 882 | CB | VAL | A | 119 | 5.696 | 58.055 | 27.106 | 1.00 | 139.37 | 6 |
| ATOM | 883 | CG1 | VAL | A | 119 | 6.478 | 59.356 | 27.069 | 1.00 | 140.66 | 6 |
| ATOM | 884 | CG2 | VAL | A | 119 | 6.640 | 56.864 | 27.142 | 1.00 | 139.26 | 6 |
| ATOM | 885 | C | VAL | A | 119 | 3.869 | 59.268 | 28.297 | 1.00 | 142.87 | 6 |
| ATOM | 886 | O | VAL | A | 119 | 3.183 | 59.505 | 27.301 | 1.00 | 143.40 | 8 |
| ATOM | 887 | N | GLU | A | 120 | 3.882 | 60.060 | 29.370 | 1.00 | 58.92 | 7 |
| ATOM | 888 | CA | GLU | A | 120 | 3.087 | 61.290 | 29.402 | 1.00 | 57.67 | 6 |
| ATOM | 889 | CB | GLU | A | 120 | 2.308 | 61.423 | 30.708 | 1.00 | 57.66 | 6 |
| ATOM | 890 | CG | GLU | A | 120 | 1.164 | 60.461 | 30.889 | 1.00 | 55.40 | 6 |
| ATOM | 891 | CD | GLU | A | 120 | 0.288 | 60.853 | 32.059 | 1.00 | 52.98 | 6 |
| ATOM | 892 | OE1 | GLU | A | 120 | −0.443 | 61.863 | 31.938 | 1.00 | 52.12 | 8 |
| ATOM | 893 | OE2 | GLU | A | 120 | 0.333 | 60.160 | 33.096 | 1.00 | 50.64 | 8 |
| ATOM | 894 | C | GLU | A | 120 | 3.947 | 62.538 | 29.255 | 1.00 | 56.66 | 6 |
| ATOM | 895 | O | GLU | A | 120 | 4.692 | 62.886 | 30.169 | 1.00 | 57.46 | 8 |
| ATOM | 896 | N | ILE | A | 121 | 3.850 | 63.209 | 28.111 | 1.00 | 43.45 | 7 |
| ATOM | 897 | CA | ILE | A | 121 | 4.604 | 64.440 | 27.898 | 1.00 | 42.84 | 6 |
| ATOM | 898 | CB | ILE | A | 121 | 4.522 | 64.902 | 26.425 | 1.00 | 53.47 | 6 |
| ATOM | 899 | CG2 | ILE | A | 121 | 5.189 | 66.249 | 26.242 | 1.00 | 53.52 | 6 |
| ATOM | 900 | CG1 | ILE | A | 121 | 5.215 | 63.875 | 25.537 | 1.00 | 53.84 | 6 |
| ATOM | 901 | CD1 | ILE | A | 121 | 6.623 | 63.569 | 25.968 | 1.00 | 54.20 | 6 |
| ATOM | 902 | C | ILE | A | 121 | 3.967 | 65.470 | 28.830 | 1.00 | 41.89 | 6 |
| ATOM | 903 | O | ILE | A | 121 | 3.431 | 66.515 | 28.406 | 1.00 | 41.93 | 8 |
| ATOM | 904 | N | MET | A | 122 | 4.020 | 65.113 | 30.112 | 1.00 | 48.93 | 7 |
| ATOM | 905 | CA | MET | A | 122 | 3.494 | 65.888 | 31.214 | 1.00 | 48.79 | 6 |
| ATOM | 906 | CB | MET | A | 122 | 4.374 | 65.665 | 32.436 | 1.00 | 96.83 | 6 |
| ATOM | 907 | CG | MET | A | 122 | 3.626 | 65.452 | 33.729 | 1.00 | 99.46 | 6 |
| ATOM | 908 | SD | MET | A | 122 | 3.284 | 63.721 | 34.020 | 1.00 | 100.08 | 16 |
| ATOM | 909 | CE | MET | A | 122 | 1.698 | 63.577 | 33.257 | 1.00 | 100.85 | 6 |
| ATOM | 910 | C | MET | A | 122 | 3.476 | 67.369 | 30.882 | 1.00 | 47.40 | 6 |
| ATOM | 911 | O | MET | A | 122 | 2.537 | 68.082 | 31.221 | 1.00 | 47.59 | 8 |
| ATOM | 912 | N | ASN | A | 123 | 4.525 | 67.822 | 30.206 | 1.00 | 100.32 | 7 |
| ATOM | 913 | CA | ASN | A | 123 | 4.660 | 69.225 | 29.861 | 1.00 | 99.15 | 6 |
| ATOM | 914 | CB | ASN | A | 123 | 5.979 | 69.765 | 30.419 | 1.00 | 60.16 | 6 |
| ATOM | 915 | CG | ASN | A | 123 | 7.193 | 68.984 | 29.924 | 1.00 | 58.49 | 6 |
| ATOM | 916 | OD1 | ASN | A | 123 | 7.088 | 67.809 | 29.560 | 1.00 | 56.34 | 8 |
| ATOM | 917 | ND2 | ASN | A | 123 | 8.357 | 69.629 | 29.941 | 1.00 | 57.81 | 7 |
| ATOM | 918 | C | ASN | A | 123 | 4.586 | 69.556 | 28.387 | 1.00 | 98.67 | 6 |
| ATOM | 919 | O | ASN | A | 123 | 5.610 | 69.847 | 27.769 | 1.00 | 99.56 | 8 |
| ATOM | 920 | N | PRO | A | 124 | 3.379 | 69.523 | 27.798 | 1.00 | 44.27 | 7 |
| ATOM | 921 | CD | PRO | A | 124 | 2.068 | 69.317 | 28.425 | 1.00 | 98.26 | 6 |
| ATOM | 922 | CA | PRO | A | 124 | 3.228 | 69.845 | 26.375 | 1.00 | 43.49 | 6 |
| ATOM | 923 | CB | PRO | A | 124 | 1.720 | 69.776 | 26.166 | 1.00 | 98.08 | 6 |
| ATOM | 924 | CG | PRO | A | 124 | 1.177 | 70.114 | 27.514 | 1.00 | 99.23 | 6 |
| ATOM | 925 | C | PRO | A | 124 | 3.809 | 71.236 | 26.091 | 1.00 | 42.28 | 6 |
| ATOM | 926 | O | PRO | A | 124 | 4.800 | 71.634 | 26.713 | 1.00 | 41.44 | 8 |
| ATOM | 927 | N | ASP | A | 125 | 3.196 | 71.987 | 25.181 | 1.00 | 72.10 | 7 |
| ATOM | 928 | CA | ASP | A | 125 | 3.715 | 73.315 | 24.842 | 1.00 | 71.22 | 6 |
| ATOM | 929 | CB | ASP | A | 125 | 3.290 | 74.367 | 25.881 | 1.00 | 58.39 | 6 |
| ATOM | 930 | CG | ASP | A | 125 | 2.384 | 73.801 | 26.968 | 1.00 | 59.12 | 6 |
| ATOM | 931 | OD1 | ASP | A | 125 | 1.510 | 72.971 | 26.649 | 1.00 | 58.91 | 8 |
| ATOM | 932 | OD2 | ASP | A | 125 | 2.528 | 74.200 | 28.145 | 1.00 | 59.76 | 8 |
| ATOM | 933 | C | ASP | A | 125 | 5.240 | 73.204 | 24.792 | 1.00 | 69.73 | 6 |
| ATOM | 934 | O | ASP | A | 125 | 5.796 | 72.731 | 23.806 | 1.00 | 70.17 | 8 |
| ATOM | 935 | N | LEU | A | 126 | 5.913 | 73.620 | 25.858 | 1.00 | 29.24 | 7 |
| ATOM | 936 | CA | LEU | A | 126 | 7.367 | 73.523 | 25.929 | 1.00 | 29.23 | 6 |
| ATOM | 937 | CB | LEU | A | 126 | 7.729 | 72.167 | 26.552 | 1.00 | 23.23 | 6 |
| ATOM | 938 | CG | LEU | A | 126 | 9.163 | 71.658 | 26.398 | 1.00 | 22.23 | 6 |
| ATOM | 939 | CD1 | LEU | A | 126 | 10.174 | 72.770 | 26.649 | 1.00 | 22.69 | 6 |
| ATOM | 940 | CD2 | LEU | A | 126 | 9.382 | 70.502 | 27.347 | 1.00 | 20.55 | 6 |
| ATOM | 941 | C | LEU | A | 126 | 8.036 | 73.707 | 24.557 | 1.00 | 29.26 | 6 |
| ATOM | 942 | O | LEU | A | 126 | 8.539 | 72.755 | 23.963 | 1.00 | 29.35 | 8 |
| ATOM | 943 | N | HIS | A | 127 | 8.045 | 74.942 | 24.062 | 1.00 | 67.12 | 7 |
| ATOM | 944 | CA | HIS | A | 127 | 8.619 | 75.246 | 22.747 | 1.00 | 68.12 | 6 |
| ATOM | 945 | CB | HIS | A | 127 | 8.936 | 76.739 | 22.638 | 1.00 | 79.55 | 6 |
| ATOM | 946 | CG | HIS | A | 127 | 9.494 | 77.146 | 21.306 | 1.00 | 81.29 | 6 |
| ATOM | 947 | CD2 | HIS | A | 127 | 10.036 | 76.418 | 20.299 | 1.00 | 81.61 | 6 |
| ATOM | 948 | ND1 | HIS | A | 127 | 9.542 | 78.461 | 20.895 | 1.00 | 82.58 | 7 |
| ATOM | 949 | CE1 | HIS | A | 127 | 10.091 | 78.526 | 19.696 | 1.00 | 82.91 | 6 |
| ATOM | 950 | NE2 | HIS | A | 127 | 10.399 | 77.301 | 19.311 | 1.00 | 82.76 | 7 |
| ATOM | 951 | C | HIS | A | 127 | 9.862 | 74.450 | 22.375 | 1.00 | 67.26 | 6 |
| ATOM | 952 | O | HIS | A | 127 | 10.961 | 74.800 | 22.780 | 1.00 | 67.73 | 8 |
| ATOM | 953 | N | ILE | A | 128 | 9.698 | 73.415 | 21.561 | 1.00 | 26.95 | 7 |
| ATOM | 954 | CA | ILE | A | 128 | 10.830 | 72.595 | 21.176 | 1.00 | 26.01 | 6 |
| ATOM | 955 | CB | ILE | A | 128 | 10.383 | 71.144 | 21.000 | 1.00 | 19.30 | 6 |
| ATOM | 956 | CG2 | ILE | A | 128 | 11.557 | 70.246 | 20.617 | 1.00 | 18.95 | 6 |
| ATOM | 957 | CG1 | ILE | A | 128 | 9.788 | 70.660 | 22.303 | 1.00 | 18.10 | 6 |
| ATOM | 958 | CD1 | ILE | A | 128 | 10.741 | 70.757 | 23.436 | 1.00 | 18.67 | 6 |
| ATOM | 959 | C | ILE | A | 128 | 11.618 | 73.032 | 19.937 | 1.00 | 26.70 | 6 |
| ATOM | 960 | O | ILE | A | 128 | 12.840 | 72.839 | 19.889 | 1.00 | 26.62 | 8 |
| ATOM | 961 | N | ALA | A | 129 | 10.947 | 73.614 | 18.939 | 1.00 | 61.74 | 7 |
| ATOM | 962 | CA | ALA | A | 129 | 11.644 | 74.037 | 17.712 | 1.00 | 63.00 | 6 |
| ATOM | 963 | CB | ALA | A | 129 | 12.309 | 72.821 | 17.053 | 1.00 | 29.93 | 6 |
| ATOM | 964 | C | ALA | A | 129 | 10.795 | 74.780 | 16.673 | 1.00 | 62.52 | 6 |
| ATOM | 965 | O | ALA | A | 129 | 10.032 | 74.176 | 15.920 | 1.00 | 62.23 | 8 |
| ATOM | 966 | N | THR | A | 130 | 10.957 | 76.095 | 16.626 | 1.00 | 42.47 | 7 |
| ATOM | 967 | CA | THR | A | 130 | 10.222 | 76.922 | 15.685 | 1.00 | 44.16 | 6 |
| ATOM | 968 | CB | THR | A | 130 | 10.261 | 78.415 | 16.114 | 1.00 | 37.02 | 6 |
| ATOM | 969 | OG1 | THR | A | 130 | 9.345 | 78.624 | 17.193 | 1.00 | 36.43 | 8 |
| ATOM | 970 | CG2 | THR | A | 130 | 9.874 | 79.329 | 14.972 | 1.00 | 38.61 | 6 |
| ATOM | 971 | C | THR | A | 130 | 10.781 | 76.773 | 14.271 | 1.00 | 44.81 | 6 |
| ATOM | 972 | O | THR | A | 130 | 11.369 | 77.711 | 13.718 | 1.00 | 45.51 | 8 |
| ATOM | 973 | N | LEU | A | 131 | 10.597 | 75.590 | 13.684 | 1.00 | 69.43 | 7 |
| ATOM | 974 | CA | LEU | A | 131 | 11.078 | 75.343 | 12.324 | 1.00 | 70.26 | 6 |
| ATOM | 975 | CB | LEU | A | 131 | 11.388 | 73.844 | 12.126 | 1.00 | 23.62 | 6 |
| ATOM | 976 | CG | LEU | A | 131 | 10.496 | 72.762 | 12.723 | 1.00 | 20.66 | 6 |
| ATOM | 977 | CD1 | LEU | A | 131 | 9.163 | 72.821 | 12.018 | 1.00 | 20.91 | 6 |
| ATOM | 978 | CD2 | LEU | A | 131 | 11.130 | 71.373 | 12.573 | 1.00 | 18.41 | 6 |
| ATOM | 979 | C | LEU | A | 131 | 10.085 | 75.865 | 11.275 | 1.00 | 71.29 | 6 |
| ATOM | 980 | O | LEU | A | 131 | 8.915 | 76.101 | 11.587 | 1.00 | 71.02 | 8 |
| ATOM | 981 | N | GLU | A | 132 | 10.552 | 76.073 | 10.045 | 1.00 | 34.39 | 7 |
| ATOM | 982 | CA | GLU | A | 132 | 9.665 | 76.587 | 9.009 | 1.00 | 37.30 | 6 |
| ATOM | 983 | CB | GLU | A | 132 | 9.125 | 77.960 | 9.414 | 1.00 | 144.24 | 6 |
| ATOM | 984 | CG | GLU | A | 132 | 10.175 | 79.051 | 9.488 | 1.00 | 146.96 | 6 |
| ATOM | 985 | CD | GLU | A | 132 | 9.600 | 80.377 | 9.947 | 1.00 | 148.66 | 6 |
| ATOM | 986 | OE1 | GLU | A | 132 | 9.088 | 80.435 | 11.084 | 1.00 | 149.93 | 8 |
| ATOM | 987 | OE2 | GLU | A | 132 | 9.656 | 81.356 | 9.174 | 1.00 | 149.02 | 8 |
| ATOM | 988 | C | GLU | A | 132 | 10.300 | 76.691 | 7.633 | 1.00 | 37.42 | 6 |
| ATOM | 989 | O | GLU | A | 132 | 11.519 | 76.702 | 7.504 | 1.00 | 36.86 | 8 |
| ATOM | 990 | N | GLU | A | 133 | 9.447 | 76.785 | 6.612 | 1.00 | 74.32 | 7 |
| ATOM | 991 | CA | GLU | A | 133 | 9.860 | 76.865 | 5.211 | 1.00 | 76.30 | 6 |
| ATOM | 992 | CB | GLU | A | 133 | 10.583 | 78.192 | 4.931 | 1.00 | 107.76 | 6 |
| ATOM | 993 | CG | GLU | A | 133 | 9.686 | 79.264 | 4.291 | 1.00 | 110.53 | 6 |
| ATOM | 994 | CD | GLU | A | 133 | 10.452 | 80.510 | 3.820 | 1.00 | 112.96 | 6 |
| ATOM | 995 | OE1 | GLU | A | 133 | 11.503 | 80.360 | 3.155 | 1.00 | 114.35 | 8 |
| ATOM | 996 | OE2 | GLU | A | 133 | 9.991 | 81.642 | 4.099 | 1.00 | 113.77 | 8 |
| ATOM | 997 | C | GLU | A | 133 | 10.735 | 75.674 | 4.796 | 1.00 | 76.32 | 6 |
| ATOM | 998 | O | GLU | A | 133 | 11.134 | 75.556 | 3.641 | 1.00 | 76.78 | 8 |
| ATOM | 999 | N | GLY | A | 134 | 11.012 | 74.786 | 5.748 | 1.00 | 49.88 | 7 |
| ATOM | 1000 | CA | GLY | A | 134 | 11.824 | 73.610 | 5.486 | 1.00 | 49.15 | 6 |
| ATOM | 1001 | C | GLY | A | 134 | 11.557 | 72.535 | 6.528 | 1.00 | 48.48 | 6 |
| ATOM | 1002 | O | GLY | A | 134 | 12.406 | 72.236 | 7.367 | 1.00 | 47.28 | 8 |
| ATOM | 1003 | N | GLY | A | 135 | 10.362 | 71.956 | 6.484 | 1.00 | 100.77 | 7 |
| ATOM | 1004 | CA | GLY | A | 135 | 10.015 | 70.916 | 7.433 | 1.00 | 100.88 | 6 |
| ATOM | 1005 | C | GLY | A | 135 | 11.060 | 69.829 | 7.445 | 1.00 | 100.39 | 6 |
| ATOM | 1006 | O | GLY | A | 135 | 11.122 | 68.995 | 6.541 | 1.00 | 101.39 | 8 |
| ATOM | 1007 | N | LYS | A | 136 | 11.889 | 69.832 | 8.477 | 1.00 | 53.75 | 7 |
| ATOM | 1008 | CA | LYS | A | 136 | 12.947 | 68.844 | 8.579 | 1.00 | 52.81 | 6 |
| ATOM | 1009 | CB | LYS | A | 136 | 14.117 | 69.281 | 7.689 | 1.00 | 74.23 | 6 |
| ATOM | 1010 | CG | LYS | A | 136 | 15.236 | 68.259 | 7.540 | 1.00 | 75.69 | 6 |
| ATOM | 1011 | CD | LYS | A | 136 | 14.739 | 66.966 | 6.914 | 1.00 | 75.71 | 6 |
| ATOM | 1012 | CE | LYS | A | 136 | 15.869 | 65.960 | 6.800 | 1.00 | 76.06 | 6 |
| ATOM | 1013 | NZ | LYS | A | 136 | 15.425 | 64.618 | 6.316 | 1.00 | 77.03 | 7 |
| ATOM | 1014 | C | LYS | A | 136 | 13.406 | 68.730 | 10.028 | 1.00 | 50.77 | 6 |
| ATOM | 1015 | O | LYS | A | 136 | 14.026 | 69.653 | 10.542 | 1.00 | 50.85 | 8 |
| ATOM | 1016 | N | LEU | A | 137 | 13.116 | 67.611 | 10.691 | 1.00 | 63.98 | 7 |
| ATOM | 1017 | CA | LEU | A | 137 | 13.541 | 67.485 | 12.074 | 1.00 | 62.44 | 6 |
| ATOM | 1018 | CB | LEU | A | 137 | 12.599 | 68.281 | 12.968 | 1.00 | 36.13 | 6 |
| ATOM | 1019 | CG | LEU | A | 137 | 13.185 | 68.463 | 14.370 | 1.00 | 34.38 | 6 |
| ATOM | 1020 | CD1 | LEU | A | 137 | 14.506 | 69.223 | 14.245 | 1.00 | 34.17 | 6 |
| ATOM | 1021 | CD2 | LEU | A | 137 | 12.211 | 69.201 | 15.277 | 1.00 | 33.90 | 6 |
| ATOM | 1022 | C | LEU | A | 137 | 13.742 | 66.105 | 12.703 | 1.00 | 61.48 | 6 |
| ATOM | 1023 | O | LEU | A | 137 | 14.491 | 65.990 | 13.667 | 1.00 | 62.25 | 8 |
| ATOM | 1024 | N | TYR | A | 138 | 13.111 | 65.063 | 12.169 | 1.00 | 28.02 | 7 |
| ATOM | 1025 | CA | TYR | A | 138 | 13.216 | 63.715 | 12.760 | 1.00 | 26.33 | 6 |
| ATOM | 1026 | CB | TYR | A | 138 | 13.480 | 62.626 | 11.710 | 1.00 | 33.29 | 6 |
| ATOM | 1027 | CG | TYR | A | 138 | 13.352 | 61.254 | 12.349 | 1.00 | 30.22 | 6 |
| ATOM | 1028 | CD1 | TYR | A | 138 | 12.109 | 60.764 | 12.741 | 1.00 | 27.96 | 6 |
| ATOM | 1029 | CE1 | TYR | A | 138 | 11.996 | 59.605 | 13.494 | 1.00 | 25.43 | 6 |
| ATOM | 1030 | CD2 | TYR | A | 138 | 14.477 | 60.528 | 12.716 | 1.00 | 28.72 | 6 |
| ATOM | 1031 | CE2 | TYR | A | 138 | 14.369 | 59.360 | 13.476 | 1.00 | 26.35 | 6 |
| ATOM | 1032 | CZ | TYR | A | 138 | 13.128 | 58.911 | 13.868 | 1.00 | 24.91 | 6 |
| ATOM | 1033 | OH | TYR | A | 138 | 13.038 | 57.813 | 14.691 | 1.00 | 22.73 | 8 |
| ATOM | 1034 | C | TYR | A | 138 | 14.234 | 63.532 | 13.878 | 1.00 | 25.01 | 6 |
| ATOM | 1035 | O | TYR | A | 138 | 15.438 | 63.496 | 13.622 | 1.00 | 23.65 | 8 |
| ATOM | 1036 | N | MET | A | 139 | 13.734 | 63.385 | 15.106 | 1.00 | 33.00 | 7 |
| ATOM | 1037 | CA | MET | A | 139 | 14.595 | 63.210 | 16.272 | 1.00 | 35.08 | 6 |
| ATOM | 1038 | CB | MET | A | 139 | 14.544 | 64.450 | 17.174 | 1.00 | 79.45 | 6 |
| ATOM | 1039 | CG | MET | A | 139 | 14.819 | 65.770 | 16.493 | 1.00 | 83.48 | 6 |
| ATOM | 1040 | SD | MET | A | 139 | 14.970 | 67.100 | 17.691 | 1.00 | 86.79 | 16 |
| ATOM | 1041 | CE | MET | A | 139 | 13.312 | 67.231 | 18.277 | 1.00 | 86.81 | 6 |
| ATOM | 1042 | C | MET | A | 139 | 14.271 | 62.000 | 17.150 | 1.00 | 34.13 | 6 |
| ATOM | 1043 | O | MET | A | 139 | 13.125 | 61.795 | 17.525 | 1.00 | 34.95 | 8 |
| ATOM | 1044 | N | GLU | A | 140 | 15.286 | 61.206 | 17.476 | 1.00 | 57.41 | 7 |
| ATOM | 1045 | CA | GLU | A | 140 | 15.118 | 60.063 | 18.367 | 1.00 | 56.32 | 6 |
| ATOM | 1046 | CB | GLU | A | 140 | 15.884 | 58.852 | 17.847 | 1.00 | 58.63 | 6 |
| ATOM | 1047 | CG | GLU | A | 140 | 15.386 | 58.247 | 16.555 | 1.00 | 58.67 | 6 |
| ATOM | 1048 | CD | GLU | A | 140 | 16.270 | 57.087 | 16.109 | 1.00 | 58.99 | 6 |
| ATOM | 1049 | OE1 | GLU | A | 140 | 16.632 | 56.255 | 16.972 | 1.00 | 58.20 | 8 |
| ATOM | 1050 | OE2 | GLU | A | 140 | 16.601 | 56.996 | 14.907 | 1.00 | 58.51 | 8 |
| ATOM | 1051 | C | GLU | A | 140 | 15.786 | 60.576 | 19.646 | 1.00 | 55.08 | 6 |
| ATOM | 1052 | O | GLU | A | 140 | 16.996 | 60.810 | 19.649 | 1.00 | 55.57 | 8 |
| ATOM | 1053 | N | VAL | A | 141 | 15.024 | 60.752 | 20.725 | 1.00 | 13.87 | 7 |
| ATOM | 1054 | CA | VAL | A | 141 | 15.589 | 61.296 | 21.968 | 1.00 | 13.87 | 6 |
| ATOM | 1055 | CB | VAL | A | 141 | 15.108 | 62.767 | 22.184 | 1.00 | 30.35 | 6 |
| ATOM | 1056 | CG1 | VAL | A | 141 | 13.773 | 63.001 | 21.501 | 1.00 | 30.27 | 6 |
| ATOM | 1057 | CG2 | VAL | A | 141 | 14.992 | 63.081 | 23.658 | 1.00 | 30.89 | 6 |
| ATOM | 1058 | C | VAL | A | 141 | 15.340 | 60.502 | 23.232 | 1.00 | 13.87 | 6 |
| ATOM | 1059 | O | VAL | A | 141 | 14.292 | 60.615 | 23.837 | 1.00 | 13.87 | 8 |
| ATOM | 1060 | N | ARG | A | 142 | 16.332 | 59.728 | 23.644 | 1.00 | 15.97 | 7 |
| ATOM | 1061 | CA | ARG | A | 142 | 16.230 | 58.885 | 24.832 | 1.00 | 17.66 | 6 |
| ATOM | 1062 | CB | ARG | A | 142 | 17.620 | 58.497 | 25.319 | 1.00 | 36.74 | 6 |
| ATOM | 1063 | CG | ARG | A | 142 | 17.799 | 57.015 | 25.461 | 1.00 | 35.26 | 6 |
| ATOM | 1064 | CD | ARG | A | 142 | 18.739 | 56.701 | 26.556 | 1.00 | 33.63 | 6 |
| ATOM | 1065 | NE | ARG | A | 142 | 18.991 | 55.274 | 26.633 | 1.00 | 33.56 | 7 |
| ATOM | 1066 | CZ | ARG | A | 142 | 19.798 | 54.715 | 27.530 | 1.00 | 35.05 | 6 |
| ATOM | 1067 | NH1 | ARG | A | 142 | 20.415 | 55.482 | 28.419 | 1.00 | 36.63 | 7 |
| ATOM | 1068 | NH2 | ARG | A | 142 | 20.006 | 53.398 | 27.540 | 1.00 | 35.26 | 7 |
| ATOM | 1069 | C | ARG | A | 142 | 15.469 | 59.522 | 25.983 | 1.00 | 18.25 | 6 |
| ATOM | 1070 | O | ARG | A | 142 | 15.072 | 60.668 | 25.876 | 1.00 | 17.69 | 8 |
| ATOM | 1071 | N | VAL | A | 143 | 15.262 | 58.765 | 27.069 | 1.00 | 52.69 | 7 |
| ATOM | 1072 | CA | VAL | A | 143 | 14.570 | 59.241 | 28.285 | 1.00 | 55.71 | 6 |
| ATOM | 1073 | CB | VAL | A | 143 | 13.054 | 59.520 | 28.052 | 1.00 | 156.86 | 6 |
| ATOM | 1074 | CG1 | VAL | A | 143 | 12.845 | 60.943 | 27.562 | 1.00 | 158.32 | 6 |
| ATOM | 1075 | CG2 | VAL | A | 143 | 12.485 | 58.534 | 27.060 | 1.00 | 159.44 | 6 |
| ATOM | 1076 | C | VAL | A | 143 | 14.678 | 58.266 | 29.463 | 1.00 | 55.82 | 6 |
| ATOM | 1077 | O | VAL | A | 143 | 13.678 | 57.742 | 29.938 | 1.00 | 56.81 | 8 |
| ATOM | 1078 | N | ASP | A | 144 | 15.903 | 58.048 | 29.933 | 1.00 | 35.56 | 7 |
| ATOM | 1079 | CA | ASP | A | 144 | 16.206 | 57.144 | 31.048 | 1.00 | 36.24 | 6 |
| ATOM | 1080 | CB | ASP | A | 144 | 17.717 | 56.993 | 31.235 | 1.00 | 43.33 | 6 |
| ATOM | 1081 | CG | ASP | A | 144 | 18.337 | 56.016 | 30.264 | 1.00 | 44.54 | 6 |
| ATOM | 1082 | OD1 | ASP | A | 144 | 17.875 | 55.965 | 29.096 | 1.00 | 44.64 | 8 |
| ATOM | 1083 | OD2 | ASP | A | 144 | 19.298 | 55.312 | 30.672 | 1.00 | 44.72 | 8 |
| ATOM | 1084 | C | ASP | A | 144 | 15.647 | 57.566 | 32.378 | 1.00 | 35.85 | 6 |
| ATOM | 1085 | O | ASP | A | 144 | 15.210 | 58.701 | 32.568 | 1.00 | 35.46 | 8 |
| ATOM | 1086 | N | ARG | A | 145 | 15.705 | 56.626 | 33.310 | 1.00 | 58.26 | 7 |
| ATOM | 1087 | CA | ARG | A | 145 | 15.223 | 56.844 | 34.659 | 1.00 | 61.18 | 6 |
| ATOM | 1088 | CB | ARG | A | 145 | 14.411 | 55.625 | 35.120 | 1.00 | 90.29 | 6 |
| ATOM | 1089 | CG | ARG | A | 145 | 13.329 | 55.909 | 36.166 | 1.00 | 92.68 | 6 |
| ATOM | 1090 | CD | ARG | A | 145 | 12.117 | 54.997 | 35.952 | 1.00 | 93.22 | 6 |
| ATOM | 1091 | NE | ARG | A | 145 | 10.965 | 55.394 | 36.759 | 1.00 | 95.42 | 7 |
| ATOM | 1092 | CZ | ARG | A | 145 | 9.702 | 55.118 | 36.438 | 1.00 | 96.41 | 6 |
| ATOM | 1093 | NH1 | ARG | A | 145 | 9.433 | 54.447 | 35.325 | 1.00 | 95.91 | 7 |
| ATOM | 1094 | NH2 | ARG | A | 145 | 8.704 | 55.509 | 37.225 | 1.00 | 97.11 | 7 |
| ATOM | 1095 | C | ARG | A | 145 | 16.487 | 56.995 | 35.484 | 1.00 | 61.07 | 6 |
| ATOM | 1096 | O | ARG | A | 145 | 17.301 | 56.077 | 35.540 | 1.00 | 61.52 | 8 |
| ATOM | 1097 | N | GLY | A | 146 | 16.665 | 58.158 | 36.103 | 1.00 | 50.34 | 7 |
| ATOM | 1098 | CA | GLY | A | 146 | 17.858 | 58.386 | 36.897 | 1.00 | 49.87 | 6 |
| ATOM | 1099 | C | GLY | A | 146 | 17.552 | 58.932 | 38.272 | 1.00 | 49.74 | 6 |
| ATOM | 1100 | O | GLY | A | 146 | 16.400 | 58.953 | 38.706 | 1.00 | 49.96 | 8 |
| ATOM | 1101 | N | VAL | A | 147 | 18.598 | 59.380 | 38.956 | 1.00 | 48.38 | 7 |
| ATOM | 1102 | CA | VAL | A | 147 | 18.475 | 59.928 | 40.300 | 1.00 | 47.31 | 6 |
| ATOM | 1103 | CB | VAL | A | 147 | 18.679 | 58.834 | 41.358 | 1.00 | 24.62 | 6 |
| ATOM | 1104 | CG1 | VAL | A | 147 | 18.986 | 59.458 | 42.698 | 1.00 | 25.16 | 6 |
| ATOM | 1105 | CG2 | VAL | A | 147 | 17.441 | 57.975 | 41.450 | 1.00 | 23.97 | 6 |
| ATOM | 1106 | C | VAL | A | 147 | 19.507 | 61.013 | 40.537 | 1.00 | 46.92 | 6 |
| ATOM | 1107 | O | VAL | A | 147 | 20.706 | 60.797 | 40.353 | 1.00 | 47.27 | 8 |
| ATOM | 1108 | N | GLY | A | 148 | 19.038 | 62.179 | 40.951 | 1.00 | 41.92 | 7 |
| ATOM | 1109 | CA | GLY | A | 148 | 19.952 | 63.271 | 41.210 | 1.00 | 42.13 | 6 |
| ATOM | 1110 | C | GLY | A | 148 | 20.387 | 63.969 | 39.941 | 1.00 | 41.88 | 6 |
| ATOM | 1111 | O | GLY | A | 148 | 19.615 | 64.060 | 38.989 | 1.00 | 41.60 | 8 |
| ATOM | 1112 | N | TYR | A | 149 | 21.633 | 64.443 | 39.931 | 1.00 | 56.71 | 7 |
| ATOM | 1113 | CA | TYR | A | 149 | 22.211 | 65.167 | 38.798 | 1.00 | 56.25 | 6 |
| ATOM | 1114 | CB | TYR | A | 149 | 22.611 | 66.564 | 39.267 | 1.00 | 33.62 | 6 |
| ATOM | 1115 | CG | TYR | A | 149 | 23.243 | 67.432 | 38.220 | 1.00 | 29.72 | 6 |
| ATOM | 1116 | CD1 | TYR | A | 149 | 22.743 | 67.479 | 36.925 | 1.00 | 27.89 | 6 |
| ATOM | 1117 | CE1 | TYR | A | 149 | 23.279 | 68.353 | 35.971 | 1.00 | 25.22 | 6 |
| ATOM | 1118 | CD2 | TYR | A | 149 | 24.300 | 68.274 | 38.542 | 1.00 | 28.71 | 6 |
| ATOM | 1119 | CE2 | TYR | A | 149 | 24.842 | 69.153 | 37.600 | 1.00 | 26.48 | 6 |
| ATOM | 1120 | CZ | TYR | A | 149 | 24.323 | 69.188 | 36.318 | 1.00 | 24.38 | 6 |
| ATOM | 1121 | OH | TYR | A | 149 | 24.840 | 70.082 | 35.409 | 1.00 | 20.70 | 8 |
| ATOM | 1122 | C | TYR | A | 149 | 23.429 | 64.449 | 38.216 | 1.00 | 57.82 | 6 |
| ATOM | 1123 | O | TYR | A | 149 | 24.454 | 64.334 | 38.884 | 1.00 | 58.18 | 8 |
| ATOM | 1124 | N | VAL | A | 150 | 23.318 | 63.971 | 36.976 | 1.00 | 25.17 | 7 |
| ATOM | 1125 | CA | VAL | A | 150 | 24.426 | 63.258 | 36.318 | 1.00 | 27.90 | 6 |
| ATOM | 1126 | CB | VAL | A | 150 | 24.146 | 61.733 | 36.207 | 1.00 | 43.10 | 6 |
| ATOM | 1127 | CG1 | VAL | A | 150 | 25.343 | 61.022 | 35.593 | 1.00 | 43.85 | 6 |
| ATOM | 1128 | CG2 | VAL | A | 150 | 23.836 | 61.150 | 37.578 | 1.00 | 43.89 | 6 |
| ATOM | 1129 | C | VAL | A | 150 | 24.683 | 63.794 | 34.915 | 1.00 | 30.48 | 6 |
| ATOM | 1130 | O | VAL | A | 150 | 23.971 | 63.450 | 33.986 | 1.00 | 31.06 | 8 |
| ATOM | 1131 | N | PRO | A | 151 | 25.715 | 64.642 | 34.754 | 1.00 | 54.43 | 7 |
| ATOM | 1132 | CD | PRO | A | 151 | 26.346 | 65.312 | 35.904 | 1.00 | 128.16 | 6 |
| ATOM | 1133 | CA | PRO | A | 151 | 26.146 | 65.283 | 33.507 | 1.00 | 57.01 | 6 |
| ATOM | 1134 | CB | PRO | A | 151 | 27.167 | 66.306 | 33.991 | 1.00 | 129.50 | 6 |
| ATOM | 1135 | CG | PRO | A | 151 | 26.658 | 66.665 | 35.336 | 1.00 | 129.68 | 6 |
| ATOM | 1136 | C | PRO | A | 151 | 26.716 | 64.407 | 32.387 | 1.00 | 59.77 | 6 |
| ATOM | 1137 | O | PRO | A | 151 | 26.354 | 63.243 | 32.226 | 1.00 | 60.40 | 8 |
| ATOM | 1138 | N | ALA | A | 152 | 27.615 | 65.001 | 31.611 | 1.00 | 72.84 | 7 |
| ATOM | 1139 | CA | ALA | A | 152 | 28.259 | 64.345 | 30.478 | 1.00 | 76.09 | 6 |
| ATOM | 1140 | CB | ALA | A | 152 | 29.206 | 65.329 | 29.774 | 1.00 | 20.79 | 6 |
| ATOM | 1141 | C | ALA | A | 152 | 29.007 | 63.063 | 30.811 | 1.00 | 78.69 | 6 |
| ATOM | 1142 | O | ALA | A | 152 | 29.763 | 62.562 | 29.983 | 1.00 | 79.51 | 8 |
| ATOM | 1143 | N | GLU | A | 153 | 28.823 | 62.534 | 32.013 | 1.00 | 107.48 | 7 |
| ATOM | 1144 | CA | GLU | A | 153 | 29.483 | 61.283 | 32.357 | 1.00 | 110.39 | 6 |
| ATOM | 1145 | CB | GLU | A | 153 | 29.558 | 61.104 | 33.869 | 1.00 | 169.79 | 6 |
| ATOM | 1146 | CG | GLU | A | 153 | 29.938 | 59.697 | 34.286 | 1.00 | 173.04 | 6 |
| ATOM | 1147 | CD | GLU | A | 153 | 30.069 | 59.550 | 35.782 | 1.00 | 175.00 | 6 |
| ATOM | 1148 | OE1 | GLU | A | 153 | 29.155 | 60.001 | 36.506 | 1.00 | 175.25 | 8 |
| ATOM | 1149 | OE2 | GLU | A | 153 | 31.082 | 58.977 | 36.237 | 1.00 | 176.25 | 8 |
| ATOM | 1150 | C | GLU | A | 153 | 28.674 | 60.135 | 31.741 | 1.00 | 110.83 | 6 |
| ATOM | 1151 | O | GLU | A | 153 | 29.210 | 59.299 | 31.013 | 1.00 | 110.62 | 8 |
| ATOM | 1152 | N | ARG | A | 154 | 27.378 | 60.107 | 32.037 | 1.00 | 163.36 | 7 |
| ATOM | 1153 | CA | ARG | A | 154 | 26.486 | 59.080 | 31.509 | 1.00 | 164.22 | 6 |
| ATOM | 1154 | CB | ARG | A | 154 | 25.102 | 59.221 | 32.158 | 1.00 | 114.78 | 6 |
| ATOM | 1155 | CG | ARG | A | 154 | 24.054 | 58.215 | 31.684 | 1.00 | 115.49 | 6 |
| ATOM | 1156 | CD | ARG | A | 154 | 22.905 | 58.046 | 32.703 | 1.00 | 115.63 | 6 |
| ATOM | 1157 | NE | ARG | A | 154 | 23.354 | 57.411 | 33.947 | 1.00 | 115.68 | 7 |
| ATOM | 1158 | CZ | ARG | A | 154 | 22.563 | 57.085 | 34.968 | 1.00 | 115.42 | 6 |
| ATOM | 1159 | NH1 | ARG | A | 154 | 21.261 | 57.329 | 34.912 | 1.00 | 114.73 | 7 |
| ATOM | 1160 | NH2 | ARG | A | 154 | 23.080 | 56.515 | 36.051 | 1.00 | 115.47 | 7 |
| ATOM | 1161 | C | ARG | A | 154 | 26.387 | 59.257 | 29.997 | 1.00 | 164.11 | 6 |
| ATOM | 1162 | O | ARG | A | 154 | 25.448 | 59.885 | 29.504 | 1.00 | 164.81 | 8 |
| ATOM | 1163 | N | HIS | A | 155 | 27.353 | 58.698 | 29.267 | 1.00 | 75.45 | 7 |
| ATOM | 1164 | CA | HIS | A | 155 | 27.383 | 58.827 | 27.810 | 1.00 | 74.77 | 6 |
| ATOM | 1165 | CB | HIS | A | 155 | 28.447 | 57.910 | 27.201 | 1.00 | 177.89 | 6 |
| ATOM | 1166 | CG | HIS | A | 155 | 28.748 | 58.216 | 25.767 | 1.00 | 178.95 | 6 |
| ATOM | 1167 | CD2 | HIS | A | 155 | 28.569 | 57.486 | 24.641 | 1.00 | 179.71 | 6 |
| ATOM | 1168 | ND1 | HIS | A | 155 | 29.281 | 59.421 | 25.361 | 1.00 | 179.47 | 7 |
| ATOM | 1169 | CE1 | HIS | A | 155 | 29.416 | 59.420 | 24.047 | 1.00 | 179.70 | 6 |
| ATOM | 1170 | NE2 | HIS | A | 155 | 28.990 | 58.258 | 23.586 | 1.00 | 179.98 | 7 |
| ATOM | 1171 | C | HIS | A | 155 | 26.034 | 58.559 | 27.148 | 1.00 | 73.51 | 6 |
| ATOM | 1172 | O | HIS | A | 155 | 25.253 | 57.708 | 27.594 | 1.00 | 72.88 | 8 |
| ATOM | 1173 | N | GLY | A | 156 | 25.775 | 59.297 | 26.073 | 1.00 | 63.99 | 7 |
| ATOM | 1174 | CA | GLY | A | 156 | 24.516 | 59.171 | 25.361 | 1.00 | 62.57 | 6 |
| ATOM | 1175 | C | GLY | A | 156 | 24.550 | 58.229 | 24.184 | 1.00 | 61.40 | 6 |
| ATOM | 1176 | O | GLY | A | 156 | 23.700 | 58.300 | 23.302 | 1.00 | 60.43 | 8 |
| ATOM | 1177 | N | ILE | A | 157 | 25.542 | 57.349 | 24.182 | 1.00 | 73.01 | 7 |
| ATOM | 1178 | CA | ILE | A | 157 | 25.707 | 56.357 | 23.135 | 1.00 | 72.98 | 6 |
| ATOM | 1179 | CB | ILE | A | 157 | 26.239 | 55.025 | 23.747 | 1.00 | 101.08 | 6 |
| ATOM | 1180 | CG2 | ILE | A | 157 | 25.403 | 54.632 | 24.964 | 1.00 | 101.52 | 6 |
| ATOM | 1181 | CG1 | ILE | A | 157 | 26.255 | 53.924 | 22.688 | 1.00 | 101.27 | 6 |
| ATOM | 1182 | CD1 | ILE | A | 157 | 26.917 | 52.644 | 23.140 | 1.00 | 102.19 | 6 |
| ATOM | 1183 | C | ILE | A | 157 | 24.409 | 56.117 | 22.353 | 1.00 | 72.00 | 6 |
| ATOM | 1184 | O | ILE | A | 157 | 24.148 | 56.808 | 21.376 | 1.00 | 71.90 | 8 |
| ATOM | 1185 | N | LYS | A | 158 | 23.588 | 55.166 | 22.795 | 1.00 | 110.17 | 7 |
| ATOM | 1186 | CA | LYS | A | 158 | 22.333 | 54.851 | 22.113 | 1.00 | 108.68 | 6 |
| ATOM | 1187 | CB | LYS | A | 158 | 21.270 | 55.916 | 22.390 | 1.00 | 97.31 | 6 |
| ATOM | 1188 | CG | LYS | A | 158 | 20.297 | 55.551 | 23.491 | 1.00 | 97.28 | 6 |
| ATOM | 1189 | CD | LYS | A | 158 | 19.567 | 54.257 | 23.177 | 1.00 | 96.09 | 6 |
| ATOM | 1190 | CE | LYS | A | 158 | 18.590 | 53.895 | 24.290 | 1.00 | 94.87 | 6 |
| ATOM | 1191 | NZ | LYS | A | 158 | 18.012 | 52.522 | 24.162 | 1.00 | 93.51 | 7 |
| ATOM | 1192 | C | LYS | A | 158 | 22.520 | 54.727 | 20.609 | 1.00 | 107.55 | 6 |
| ATOM | 1193 | O | LYS | A | 158 | 22.982 | 53.697 | 20.115 | 1.00 | 108.08 | 8 |
| ATOM | 1194 | N | ASP | A | 159 | 22.164 | 55.790 | 19.889 | 1.00 | 59.19 | 7 |
| ATOM | 1195 | CA | ASP | A | 159 | 22.263 | 55.839 | 18.426 | 1.00 | 57.80 | 6 |
| ATOM | 1196 | CB | ASP | A | 159 | 21.589 | 57.125 | 17.907 | 1.00 | 86.33 | 6 |
| ATOM | 1197 | CG | ASP | A | 159 | 21.011 | 56.968 | 16.505 | 1.00 | 85.93 | 6 |
| ATOM | 1198 | OD1 | ASP | A | 159 | 21.796 | 56.858 | 15.544 | 1.00 | 86.75 | 8 |
| ATOM | 1199 | OD2 | ASP | A | 159 | 19.768 | 56.950 | 16.363 | 1.00 | 84.74 | 8 |
| ATOM | 1200 | C | ASP | A | 159 | 23.723 | 55.786 | 17.983 | 1.00 | 56.46 | 6 |
| ATOM | 1201 | O | ASP | A | 159 | 24.054 | 56.164 | 16.866 | 1.00 | 55.59 | 8 |
| ATOM | 1202 | N | ARG | A | 160 | 24.582 | 55.311 | 18.881 | 1.00 | 87.56 | 7 |
| ATOM | 1203 | CA | ARG | A | 160 | 26.014 | 55.182 | 18.631 | 1.00 | 88.42 | 6 |
| ATOM | 1204 | CB | ARG | A | 160 | 26.402 | 53.702 | 18.522 | 1.00 | 102.75 | 6 |
| ATOM | 1205 | CG | ARG | A | 160 | 26.715 | 53.008 | 19.844 | 1.00 | 102.79 | 6 |
| ATOM | 1206 | CD | ARG | A | 160 | 27.249 | 51.598 | 19.603 | 1.00 | 102.12 | 6 |
| ATOM | 1207 | NE | ARG | A | 160 | 27.592 | 50.890 | 20.833 | 1.00 | 100.58 | 7 |
| ATOM | 1208 | CZ | ARG | A | 160 | 28.112 | 49.668 | 20.860 | 1.00 | 100.56 | 6 |
| ATOM | 1209 | NH1 | ARG | A | 160 | 28.347 | 49.025 | 19.725 | 1.00 | 100.25 | 7 |
| ATOM | 1210 | NH2 | ARG | A | 160 | 28.400 | 49.085 | 22.014 | 1.00 | 100.27 | 7 |
| ATOM | 1211 | C | ARG | A | 160 | 26.528 | 55.907 | 17.393 | 1.00 | 87.57 | 6 |
| ATOM | 1212 | O | ARG | A | 160 | 27.015 | 55.271 | 16.456 | 1.00 | 88.01 | 8 |
| ATOM | 1213 | N | ILE | A | 161 | 26.424 | 57.230 | 17.374 | 1.00 | 106.49 | 7 |
| ATOM | 1214 | CA | ILE | A | 161 | 26.924 | 57.961 | 16.222 | 1.00 | 104.57 | 6 |
| ATOM | 1215 | CB | ILE | A | 161 | 26.176 | 57.570 | 14.931 | 1.00 | 48.58 | 6 |
| ATOM | 1216 | CG2 | ILE | A | 161 | 24.709 | 57.935 | 15.052 | 1.00 | 47.75 | 6 |
| ATOM | 1217 | CG1 | ILE | A | 161 | 26.816 | 58.289 | 13.731 | 1.00 | 48.28 | 6 |
| ATOM | 1218 | CD1 | ILE | A | 161 | 26.382 | 57.777 | 12.355 | 1.00 | 48.64 | 6 |
| ATOM | 1219 | C | ILE | A | 161 | 26.851 | 59.464 | 16.347 | 1.00 | 103.31 | 6 |
| ATOM | 1220 | O | ILE | A | 161 | 27.860 | 60.161 | 16.218 | 1.00 | 103.93 | 8 |
| ATOM | 1221 | N | ASN | A | 162 | 25.651 | 59.964 | 16.597 | 1.00 | 73.05 | 7 |
| ATOM | 1222 | CA | ASN | A | 162 | 25.453 | 61.396 | 16.686 | 1.00 | 70.40 | 6 |
| ATOM | 1223 | CB | ASN | A | 162 | 24.800 | 61.873 | 15.392 | 1.00 | 45.57 | 6 |
| ATOM | 1224 | CG | ASN | A | 162 | 25.310 | 61.112 | 14.168 | 1.00 | 45.25 | 6 |
| ATOM | 1225 | OD1 | ASN | A | 162 | 26.494 | 61.185 | 13.807 | 1.00 | 44.13 | 8 |
| ATOM | 1226 | ND2 | ASN | A | 162 | 24.412 | 60.371 | 13.528 | 1.00 | 44.20 | 7 |
| ATOM | 1227 | C | ASN | A | 162 | 24.569 | 61.701 | 17.876 | 1.00 | 68.28 | 6 |
| ATOM | 1228 | O | ASN | A | 162 | 23.898 | 62.728 | 17.924 | 1.00 | 68.79 | 8 |
| ATOM | 1229 | N | ALA | A | 163 | 24.586 | 60.787 | 18.837 | 1.00 | 49.85 | 7 |
| ATOM | 1230 | CA | ALA | A | 163 | 23.784 | 60.911 | 20.039 | 1.00 | 46.49 | 6 |
| ATOM | 1231 | CB | ALA | A | 163 | 24.181 | 59.824 | 21.022 | 1.00 | 113.93 | 6 |
| ATOM | 1232 | C | ALA | A | 163 | 23.876 | 62.279 | 20.709 | 1.00 | 43.84 | 6 |
| ATOM | 1233 | O | ALA | A | 163 | 22.933 | 62.712 | 21.366 | 1.00 | 42.33 | 8 |
| ATOM | 1234 | N | ILE | A | 164 | 25.003 | 62.960 | 20.550 | 1.00 | 44.78 | 7 |
| ATOM | 1235 | CA | ILE | A | 164 | 25.183 | 64.267 | 21.171 | 1.00 | 41.25 | 6 |
| ATOM | 1236 | CB | ILE | A | 164 | 24.538 | 65.390 | 20.309 | 1.00 | 14.84 | 6 |
| ATOM | 1237 | CG2 | ILE | A | 164 | 24.860 | 65.165 | 18.847 | 1.00 | 13.87 | 6 |
| ATOM | 1238 | CG1 | ILE | A | 164 | 23.023 | 65.399 | 20.465 | 1.00 | 13.87 | 6 |
| ATOM | 1239 | CD1 | ILE | A | 164 | 22.358 | 66.472 | 19.666 | 1.00 | 13.87 | 6 |
| ATOM | 1240 | C | ILE | A | 164 | 24.560 | 64.289 | 22.570 | 1.00 | 40.59 | 6 |
| ATOM | 1241 | O | ILE | A | 164 | 23.845 | 65.219 | 22.913 | 1.00 | 41.32 | 8 |
| ATOM | 1242 | N | PRO | A | 165 | 24.867 | 63.282 | 23.407 | 1.00 | 38.68 | 7 |
| ATOM | 1243 | CD | PRO | A | 165 | 26.175 | 62.626 | 23.304 | 1.00 | 32.21 | 6 |
| ATOM | 1244 | CA | PRO | A | 165 | 24.358 | 63.127 | 24.778 | 1.00 | 37.84 | 6 |
| ATOM | 1245 | CB | PRO | A | 165 | 25.469 | 62.357 | 25.478 | 1.00 | 31.90 | 6 |
| ATOM | 1246 | CG | PRO | A | 165 | 26.670 | 62.746 | 24.710 | 1.00 | 31.31 | 6 |
| ATOM | 1247 | C | PRO | A | 165 | 24.057 | 64.433 | 25.473 | 1.00 | 38.07 | 6 |
| ATOM | 1248 | O | PRO | A | 165 | 24.861 | 65.366 | 25.398 | 1.00 | 38.33 | 8 |
| ATOM | 1249 | N | VAL | A | 166 | 22.908 | 64.488 | 26.152 | 1.00 | 28.76 | 7 |
| ATOM | 1250 | CA | VAL | A | 166 | 22.462 | 65.687 | 26.869 | 1.00 | 28.17 | 6 |
| ATOM | 1251 | CB | VAL | A | 166 | 21.018 | 66.014 | 26.538 | 1.00 | 37.29 | 6 |
| ATOM | 1252 | CG1 | VAL | A | 166 | 20.639 | 67.328 | 27.174 | 1.00 | 37.95 | 6 |
| ATOM | 1253 | CG2 | VAL | A | 166 | 20.830 | 66.045 | 25.056 | 1.00 | 37.42 | 6 |
| ATOM | 1254 | C | VAL | A | 166 | 22.532 | 65.548 | 28.384 | 1.00 | 28.88 | 6 |
| ATOM | 1255 | O | VAL | A | 166 | 22.344 | 64.452 | 28.927 | 1.00 | 29.93 | 8 |
| ATOM | 1256 | N | ASP | A | 167 | 22.780 | 66.659 | 29.070 | 1.00 | 27.15 | 7 |
| ATOM | 1257 | CA | ASP | A | 167 | 22.847 | 66.627 | 30.525 | 1.00 | 28.82 | 6 |
| ATOM | 1258 | CB | ASP | A | 167 | 23.131 | 68.019 | 31.079 | 1.00 | 55.43 | 6 |
| ATOM | 1259 | CG | ASP | A | 167 | 24.512 | 68.503 | 30.750 | 1.00 | 56.58 | 6 |
| ATOM | 1260 | OD1 | ASP | A | 167 | 25.452 | 67.682 | 30.781 | 1.00 | 57.79 | 8 |
| ATOM | 1261 | OD2 | ASP | A | 167 | 24.654 | 69.709 | 30.478 | 1.00 | 55.60 | 8 |
| ATOM | 1262 | C | ASP | A | 167 | 21.540 | 66.119 | 31.133 | 1.00 | 28.92 | 6 |
| ATOM | 1263 | O | ASP | A | 167 | 20.459 | 66.350 | 30.589 | 1.00 | 29.75 | 8 |
| ATOM | 1264 | N | ALA | A | 168 | 21.632 | 65.448 | 32.271 | 1.00 | 33.33 | 7 |
| ATOM | 1265 | CA | ALA | A | 168 | 20.445 | 64.928 | 32.920 | 1.00 | 35.59 | 6 |
| ATOM | 1266 | CB | ALA | A | 168 | 20.649 | 63.459 | 33.243 | 1.00 | 46.87 | 6 |
| ATOM | 1267 | C | ALA | A | 168 | 20.098 | 65.700 | 34.193 | 1.00 | 37.01 | 6 |
| ATOM | 1268 | O | ALA | A | 168 | 20.974 | 66.214 | 34.871 | 1.00 | 37.01 | 8 |
| ATOM | 1269 | N | ILE | A | 169 | 18.810 | 65.789 | 34.500 | 1.00 | 41.48 | 7 |
| ATOM | 1270 | CA | ILE | A | 169 | 18.325 | 66.460 | 35.702 | 1.00 | 43.82 | 6 |
| ATOM | 1271 | CB | ILE | A | 169 | 17.784 | 67.873 | 35.411 | 1.00 | 66.79 | 6 |
| ATOM | 1272 | CG2 | ILE | A | 169 | 17.951 | 68.745 | 36.637 | 1.00 | 67.21 | 6 |
| ATOM | 1273 | CG1 | ILE | A | 169 | 18.515 | 68.500 | 34.224 | 1.00 | 68.86 | 6 |
| ATOM | 1274 | CD1 | ILE | A | 169 | 19.952 | 68.843 | 34.495 | 1.00 | 71.35 | 6 |
| ATOM | 1275 | C | ILE | A | 169 | 17.141 | 65.592 | 36.096 | 1.00 | 44.59 | 6 |
| ATOM | 1276 | O | ILE | A | 169 | 16.015 | 65.859 | 35.670 | 1.00 | 45.12 | 8 |
| ATOM | 1277 | N | PHE | A | 170 | 17.395 | 64.559 | 36.895 | 1.00 | 61.53 | 7 |
| ATOM | 1278 | CA | PHE | A | 170 | 16.356 | 63.613 | 37.308 | 1.00 | 63.83 | 6 |
| ATOM | 1279 | CB | PHE | A | 170 | 17.021 | 62.303 | 37.715 | 1.00 | 95.45 | 6 |
| ATOM | 1280 | CG | PHE | A | 170 | 17.810 | 61.673 | 36.614 | 1.00 | 98.05 | 6 |
| ATOM | 1281 | CD1 | PHE | A | 170 | 17.184 | 61.259 | 35.450 | 1.00 | 97.88 | 6 |
| ATOM | 1282 | CD2 | PHE | A | 170 | 19.182 | 61.512 | 36.728 | 1.00 | 99.39 | 6 |
| ATOM | 1283 | CE1 | PHE | A | 170 | 17.914 | 60.693 | 34.418 | 1.00 | 97.26 | 6 |
| ATOM | 1284 | CE2 | PHE | A | 170 | 19.921 | 60.946 | 35.699 | 1.00 | 98.86 | 6 |
| ATOM | 1285 | CZ | PHE | A | 170 | 19.288 | 60.539 | 34.544 | 1.00 | 97.30 | 6 |
| ATOM | 1286 | C | PHE | A | 170 | 15.333 | 64.023 | 38.378 | 1.00 | 64.23 | 6 |
| ATOM | 1287 | O | PHE | A | 170 | 14.134 | 63.787 | 38.207 | 1.00 | 64.72 | 8 |
| ATOM | 1288 | N | SER | A | 171 | 15.785 | 64.620 | 39.476 | 1.00 | 39.73 | 7 |
| ATOM | 1289 | CA | SER | A | 171 | 14.868 | 65.024 | 40.540 | 1.00 | 38.09 | 6 |
| ATOM | 1290 | CB | SER | A | 171 | 15.535 | 66.065 | 41.444 | 1.00 | 78.49 | 6 |
| ATOM | 1291 | OG | SER | A | 171 | 14.693 | 66.419 | 42.525 | 1.00 | 80.28 | 8 |
| ATOM | 1292 | C | SER | A | 171 | 13.556 | 65.592 | 39.987 | 1.00 | 35.93 | 6 |
| ATOM | 1293 | O | SER | A | 171 | 13.555 | 66.540 | 39.201 | 1.00 | 36.51 | 8 |
| ATOM | 1294 | N | PRO | A | 172 | 12.419 | 64.991 | 40.369 | 1.00 | 48.33 | 7 |
| ATOM | 1295 | CD | PRO | A | 172 | 12.324 | 63.681 | 41.039 | 1.00 | 44.87 | 6 |
| ATOM | 1296 | CA | PRO | A | 172 | 11.098 | 65.448 | 39.916 | 1.00 | 47.93 | 6 |
| ATOM | 1297 | CB | PRO | A | 172 | 10.238 | 64.206 | 40.068 | 1.00 | 43.94 | 6 |
| ATOM | 1298 | CG | PRO | A | 172 | 10.838 | 63.550 | 41.291 | 1.00 | 44.29 | 6 |
| ATOM | 1299 | C | PRO | A | 172 | 10.631 | 66.582 | 40.814 | 1.00 | 46.83 | 6 |
| ATOM | 1300 | O | PRO | A | 172 | 9.736 | 67.335 | 40.472 | 1.00 | 47.00 | 8 |
| ATOM | 1301 | N | VAL | A | 173 | 11.252 | 66.679 | 41.982 | 1.00 | 41.35 | 7 |
| ATOM | 1302 | CA | VAL | A | 173 | 10.950 | 67.721 | 42.952 | 1.00 | 40.25 | 6 |
| ATOM | 1303 | CB | VAL | A | 173 | 11.693 | 67.420 | 44.270 | 1.00 | 29.43 | 6 |
| ATOM | 1304 | CG1 | VAL | A | 173 | 11.439 | 68.512 | 45.283 | 1.00 | 30.41 | 6 |
| ATOM | 1305 | CG2 | VAL | A | 173 | 11.245 | 66.063 | 44.815 | 1.00 | 27.70 | 6 |
| ATOM | 1306 | C | VAL | A | 173 | 11.445 | 69.031 | 42.342 | 1.00 | 39.45 | 6 |
| ATOM | 1307 | O | VAL | A | 173 | 12.603 | 69.127 | 41.955 | 1.00 | 39.32 | 8 |
| ATOM | 1308 | N | ARG | A | 174 | 10.589 | 70.038 | 42.237 | 1.00 | 59.70 | 7 |
| ATOM | 1309 | CA | ARG | A | 174 | 11.034 | 71.288 | 41.625 | 1.00 | 62.26 | 6 |
| ATOM | 1310 | CB | ARG | A | 174 | 10.105 | 71.677 | 40.469 | 1.00 | 140.03 | 6 |
| ATOM | 1311 | CG | ARG | A | 174 | 10.243 | 70.821 | 39.208 | 1.00 | 144.79 | 6 |
| ATOM | 1312 | CD | ARG | A | 174 | 11.541 | 71.100 | 38.433 | 1.00 | 149.38 | 6 |
| ATOM | 1313 | NE | ARG | A | 174 | 11.635 | 70.294 | 37.212 | 1.00 | 151.23 | 7 |
| ATOM | 1314 | CZ | ARG | A | 174 | 12.669 | 70.302 | 36.373 | 1.00 | 151.78 | 6 |
| ATOM | 1315 | NH1 | ARG | A | 174 | 13.719 | 71.079 | 36.609 | 1.00 | 152.98 | 7 |
| ATOM | 1316 | NH2 | ARG | A | 174 | 12.658 | 69.523 | 35.298 | 1.00 | 151.43 | 7 |
| ATOM | 1317 | C | ARG | A | 174 | 11.190 | 72.490 | 42.555 | 1.00 | 61.59 | 6 |
| ATOM | 1318 | O | ARG | A | 174 | 11.310 | 73.619 | 42.079 | 1.00 | 60.56 | 8 |
| ATOM | 1319 | N | ARG | A | 175 | 11.207 | 72.252 | 43.865 | 1.00 | 50.49 | 7 |
| ATOM | 1320 | CA | ARG | A | 175 | 11.345 | 73.323 | 44.853 | 1.00 | 48.86 | 6 |
| ATOM | 1321 | CB | ARG | A | 175 | 10.736 | 74.609 | 44.312 | 1.00 | 54.88 | 6 |
| ATOM | 1322 | CG | ARG | A | 175 | 10.585 | 75.698 | 45.317 | 1.00 | 54.77 | 6 |
| ATOM | 1323 | CD | ARG | A | 175 | 10.180 | 76.974 | 44.623 | 1.00 | 53.43 | 6 |
| ATOM | 1324 | NE | ARG | A | 175 | 9.537 | 77.896 | 45.545 | 1.00 | 53.72 | 7 |
| ATOM | 1325 | CZ | ARG | A | 175 | 9.203 | 79.137 | 45.232 | 1.00 | 53.04 | 6 |
| ATOM | 1326 | NH1 | ARG | A | 175 | 9.464 | 79.596 | 44.017 | 1.00 | 53.52 | 7 |
| ATOM | 1327 | NH2 | ARG | A | 175 | 8.591 | 79.908 | 46.122 | 1.00 | 52.20 | 7 |
| ATOM | 1328 | C | ARG | A | 175 | 10.610 | 72.895 | 46.109 | 1.00 | 48.09 | 6 |
| ATOM | 1329 | O | ARG | A | 175 | 9.603 | 72.218 | 46.010 | 1.00 | 49.40 | 8 |
| ATOM | 1330 | N | VAL | A | 176 | 11.104 | 73.282 | 47.282 | 1.00 | 34.91 | 7 |
| ATOM | 1331 | CA | VAL | A | 176 | 10.473 | 72.902 | 48.552 | 1.00 | 35.21 | 6 |
| ATOM | 1332 | CB | VAL | A | 176 | 10.989 | 71.547 | 49.077 | 1.00 | 13.87 | 6 |
| ATOM | 1333 | CG1 | VAL | A | 176 | 10.037 | 71.013 | 50.122 | 1.00 | 13.87 | 6 |
| ATOM | 1334 | CG2 | VAL | A | 176 | 11.208 | 70.567 | 47.947 | 1.00 | 13.87 | 6 |
| ATOM | 1335 | C | VAL | A | 176 | 10.769 | 73.881 | 49.683 | 1.00 | 36.88 | 6 |
| ATOM | 1336 | O | VAL | A | 176 | 11.909 | 74.302 | 49.860 | 1.00 | 37.11 | 8 |
| ATOM | 1337 | N | ALA | A | 177 | 9.752 | 74.213 | 50.468 | 1.00 | 45.24 | 7 |
| ATOM | 1338 | CA | ALA | A | 177 | 9.925 | 75.106 | 51.619 | 1.00 | 47.00 | 6 |
| ATOM | 1339 | CB | ALA | A | 177 | 9.479 | 76.520 | 51.272 | 1.00 | 36.43 | 6 |
| ATOM | 1340 | C | ALA | A | 177 | 9.082 | 74.553 | 52.769 | 1.00 | 48.64 | 6 |
| ATOM | 1341 | O | ALA | A | 177 | 8.533 | 73.459 | 52.661 | 1.00 | 48.85 | 8 |
| ATOM | 1342 | N | PHE | A | 178 | 8.983 | 75.282 | 53.873 | 1.00 | 98.12 | 7 |
| ATOM | 1343 | CA | PHE | A | 178 | 8.156 | 74.790 | 54.961 | 1.00 | 100.59 | 6 |
| ATOM | 1344 | CB | PHE | A | 178 | 8.641 | 73.404 | 55.393 | 1.00 | 40.54 | 6 |
| ATOM | 1345 | CG | PHE | A | 178 | 9.908 | 73.401 | 56.184 | 1.00 | 42.13 | 6 |
| ATOM | 1346 | CD1 | PHE | A | 178 | 9.878 | 73.537 | 57.567 | 1.00 | 42.94 | 6 |
| ATOM | 1347 | CD2 | PHE | A | 178 | 11.126 | 73.173 | 55.563 | 1.00 | 43.85 | 6 |
| ATOM | 1348 | CE1 | PHE | A | 178 | 11.046 | 73.437 | 58.325 | 1.00 | 46.29 | 6 |
| ATOM | 1349 | CE2 | PHE | A | 178 | 12.304 | 73.071 | 56.311 | 1.00 | 46.44 | 6 |
| ATOM | 1350 | CZ | PHE | A | 178 | 12.263 | 73.200 | 57.696 | 1.00 | 47.46 | 6 |
| ATOM | 1351 | C | PHE | A | 178 | 8.020 | 75.719 | 56.154 | 1.00 | 103.23 | 6 |
| ATOM | 1352 | O | PHE | A | 178 | 8.988 | 75.976 | 56.866 | 1.00 | 105.47 | 8 |
| ATOM | 1353 | N | GLN | A | 179 | 6.803 | 76.224 | 56.359 | 1.00 | 36.08 | 7 |
| ATOM | 1354 | CA | GLN | A | 179 | 6.512 | 77.136 | 57.459 | 1.00 | 37.00 | 6 |
| ATOM | 1355 | CB | GLN | A | 179 | 5.137 | 77.766 | 57.254 | 1.00 | 92.61 | 6 |
| ATOM | 1356 | CG | GLN | A | 179 | 4.987 | 78.522 | 55.951 | 1.00 | 96.35 | 6 |
| ATOM | 1357 | CD | GLN | A | 179 | 5.787 | 79.805 | 55.924 | 1.00 | 99.08 | 6 |
| ATOM | 1358 | OE1 | GLN | A | 179 | 7.015 | 79.782 | 55.942 | 1.00 | 101.45 | 8 |
| ATOM | 1359 | NE2 | GLN | A | 179 | 5.090 | 80.938 | 55.889 | 1.00 | 99.95 | 7 |
| ATOM | 1360 | C | GLN | A | 179 | 6.536 | 76.385 | 58.784 | 1.00 | 37.83 | 6 |
| ATOM | 1361 | O | GLN | A | 179 | 6.288 | 75.183 | 58.822 | 1.00 | 38.53 | 8 |
| ATOM | 1362 | N | VAL | A | 180 | 6.852 | 77.085 | 59.869 | 1.00 | 74.13 | 7 |
| ATOM | 1363 | CA | VAL | A | 180 | 6.873 | 76.453 | 61.180 | 1.00 | 76.69 | 6 |
| ATOM | 1364 | CB | VAL | A | 180 | 8.290 | 76.399 | 61.781 | 1.00 | 62.85 | 6 |
| ATOM | 1365 | CG1 | VAL | A | 180 | 8.292 | 75.548 | 63.055 | 1.00 | 62.38 | 6 |
| ATOM | 1366 | CG2 | VAL | A | 180 | 9.256 | 75.814 | 60.773 | 1.00 | 63.79 | 6 |
| ATOM | 1367 | C | VAL | A | 180 | 5.977 | 77.248 | 62.110 | 1.00 | 78.88 | 6 |
| ATOM | 1368 | O | VAL | A | 180 | 6.244 | 77.347 | 63.307 | 1.00 | 79.49 | 8 |
| ATOM | 1369 | N | GLU | A | 181 | 4.912 | 77.809 | 61.539 | 1.00 | 79.29 | 7 |
| ATOM | 1370 | CA | GLU | A | 181 | 3.934 | 78.610 | 62.270 | 1.00 | 81.32 | 6 |
| ATOM | 1371 | CB | GLU | A | 181 | 2.627 | 78.668 | 61.501 | 1.00 | 65.76 | 6 |
| ATOM | 1372 | CG | GLU | A | 181 | 2.766 | 79.155 | 60.093 | 1.00 | 67.56 | 6 |
| ATOM | 1373 | CD | GLU | A | 181 | 1.498 | 78.947 | 59.293 | 1.00 | 68.61 | 6 |
| ATOM | 1374 | OE1 | GLU | A | 181 | 0.434 | 79.440 | 59.728 | 1.00 | 69.63 | 8 |
| ATOM | 1375 | OE2 | GLU | A | 181 | 1.565 | 78.289 | 58.231 | 1.00 | 68.52 | 8 |
| ATOM | 1376 | C | GLU | A | 181 | 3.627 | 78.068 | 63.652 | 1.00 | 83.63 | 6 |
| ATOM | 1377 | O | GLU | A | 181 | 3.874 | 76.899 | 63.955 | 1.00 | 84.41 | 8 |
| ATOM | 1378 | N | ASP | A | 182 | 3.071 | 78.937 | 64.483 | 1.00 | 72.84 | 7 |
| ATOM | 1379 | CA | ASP | A | 182 | 2.684 | 78.576 | 65.833 | 1.00 | 75.42 | 6 |
| ATOM | 1380 | CB | ASP | A | 182 | 3.601 | 79.262 | 66.848 | 1.00 | 117.68 | 6 |
| ATOM | 1381 | CG | ASP | A | 182 | 3.544 | 80.775 | 66.762 | 1.00 | 118.15 | 6 |
| ATOM | 1382 | OD1 | ASP | A | 182 | 3.541 | 81.309 | 65.631 | 1.00 | 117.09 | 8 |
| ATOM | 1383 | OD2 | ASP | A | 182 | 3.514 | 81.427 | 67.828 | 1.00 | 117.92 | 8 |
| ATOM | 1384 | C | ASP | A | 182 | 1.252 | 79.068 | 65.974 | 1.00 | 77.55 | 6 |
| ATOM | 1385 | O | ASP | A | 182 | 1.010 | 80.273 | 66.022 | 1.00 | 78.76 | 8 |
| ATOM | 1386 | N | THR | A | 183 | 0.303 | 78.136 | 65.998 | 1.00 | 102.76 | 7 |
| ATOM | 1387 | CA | THR | A | 183 | −1.110 | 78.488 | 66.117 | 1.00 | 104.69 | 6 |
| ATOM | 1388 | CB | THR | A | 183 | −1.682 | 79.018 | 64.790 | 1.00 | 68.16 | 6 |
| ATOM | 1389 | OG1 | THR | A | 183 | −3.077 | 79.308 | 64.966 | 1.00 | 69.31 | 8 |
| ATOM | 1390 | CG2 | THR | A | 183 | −1.505 | 77.982 | 63.670 | 1.00 | 68.00 | 6 |
| ATOM | 1391 | C | THR | A | 183 | −1.938 | 77.285 | 66.519 | 1.00 | 106.50 | 6 |
| ATOM | 1392 | O | THR | A | 183 | −2.896 | 76.922 | 65.835 | 1.00 | 106.41 | 8 |
| ATOM | 1393 | N | ARG | A | 184 | −1.575 | 76.674 | 67.638 | 1.00 | 139.74 | 7 |
| ATOM | 1394 | CA | ARG | A | 184 | −2.292 | 75.505 | 68.101 | 1.00 | 141.90 | 6 |
| ATOM | 1395 | CB | ARG | A | 184 | −1.407 | 74.268 | 67.905 | 1.00 | 97.52 | 6 |
| ATOM | 1396 | CG | ARG | A | 184 | −0.950 | 74.092 | 66.450 | 1.00 | 98.60 | 6 |
| ATOM | 1397 | CD | ARG | A | 184 | −2.150 | 74.213 | 65.501 | 1.00 | 99.55 | 6 |
| ATOM | 1398 | NE | ARG | A | 184 | −1.814 | 74.104 | 64.082 | 1.00 | 100.02 | 7 |
| ATOM | 1399 | CZ | ARG | A | 184 | −2.697 | 74.265 | 63.100 | 1.00 | 99.96 | 6 |
| ATOM | 1400 | NH1 | ARG | A | 184 | −3.961 | 74.545 | 63.382 | 1.00 | 99.41 | 7 |
| ATOM | 1401 | NH2 | ARG | A | 184 | −2.326 | 74.127 | 61.837 | 1.00 | 100.11 | 7 |
| ATOM | 1402 | C | ARG | A | 184 | −2.717 | 75.663 | 69.554 | 1.00 | 142.96 | 6 |
| ATOM | 1403 | O | ARG | A | 184 | −2.285 | 76.593 | 70.232 | 1.00 | 143.20 | 8 |
| ATOM | 1404 | N | ALA | A | 185 | −3.578 | 74.769 | 70.026 | 1.00 | 187.75 | 7 |
| ATOM | 1405 | CA | ALA | A | 185 | −4.043 | 74.836 | 71.405 | 1.00 | 188.97 | 6 |
| ATOM | 1406 | CB | ALA | A | 185 | −5.543 | 74.605 | 71.462 | 1.00 | 138.79 | 6 |
| ATOM | 1407 | C | ALA | A | 185 | −3.316 | 73.806 | 72.260 | 1.00 | 189.46 | 6 |
| ATOM | 1408 | O | ALA | A | 185 | −3.080 | 74.034 | 73.448 | 1.00 | 190.37 | 8 |
| ATOM | 1409 | N | GLY | A | 186 | −2.968 | 72.680 | 71.639 | 1.00 | 113.15 | 7 |
| ATOM | 1410 | CA | GLY | A | 186 | −2.262 | 71.609 | 72.321 | 1.00 | 111.99 | 6 |
| ATOM | 1411 | C | GLY | A | 186 | −2.071 | 71.790 | 73.815 | 1.00 | 112.66 | 6 |
| ATOM | 1412 | O | GLY | A | 186 | −2.942 | 71.421 | 74.601 | 1.00 | 112.80 | 8 |
| ATOM | 1413 | N | GLN | A | 187 | −0.934 | 72.360 | 74.209 | 1.00 | 115.28 | 7 |
| ATOM | 1414 | CA | GLN | A | 187 | −0.628 | 72.584 | 75.621 | 1.00 | 115.21 | 6 |
| ATOM | 1415 | CB | GLN | A | 187 | −0.019 | 71.318 | 76.236 | 1.00 | 172.74 | 6 |
| ATOM | 1416 | CG | GLN | A | 187 | −0.943 | 70.103 | 76.187 | 1.00 | 174.52 | 6 |
| ATOM | 1417 | CD | GLN | A | 187 | −0.394 | 68.908 | 76.942 | 1.00 | 175.30 | 6 |
| ATOM | 1418 | OE1 | GLN | A | 187 | 0.693 | 68.415 | 76.643 | 1.00 | 176.13 | 8 |
| ATOM | 1419 | NE2 | GLN | A | 187 | −1.149 | 68.431 | 77.924 | 1.00 | 175.29 | 7 |
| ATOM | 1420 | C | GLN | A | 187 | 0.338 | 73.752 | 75.790 | 1.00 | 115.15 | 6 |
| ATOM | 1421 | O | GLN | A | 187 | 1.550 | 73.580 | 75.696 | 1.00 | 115.42 | 8 |
| ATOM | 1422 | N | ARG | A | 188 | −0.207 | 74.936 | 76.049 | 1.00 | 172.13 | 7 |
| ATOM | 1423 | CA | ARG | A | 188 | 0.599 | 76.141 | 76.227 | 1.00 | 170.84 | 6 |
| ATOM | 1424 | CB | ARG | A | 188 | 1.701 | 75.908 | 77.268 | 1.00 | 192.51 | 6 |
| ATOM | 1425 | CG | ARG | A | 188 | 2.709 | 77.045 | 77.374 | 1.00 | 194.75 | 6 |
| ATOM | 1426 | CD | ARG | A | 188 | 3.794 | 76.741 | 78.391 | 1.00 | 197.08 | 6 |
| ATOM | 1427 | NE | ARG | A | 188 | 4.826 | 77.771 | 78.390 | 1.00 | 200.84 | 7 |
| ATOM | 1428 | CZ | ARG | A | 188 | 5.871 | 77.781 | 79.208 | 1.00 | 202.51 | 6 |
| ATOM | 1429 | NH1 | ARG | A | 188 | 6.025 | 76.811 | 80.100 | 1.00 | 204.00 | 7 |
| ATOM | 1430 | NH2 | ARG | A | 188 | 6.765 | 78.759 | 79.134 | 1.00 | 202.86 | 7 |
| ATOM | 1431 | C | ARG | A | 188 | 1.230 | 76.544 | 74.903 | 1.00 | 169.28 | 6 |
| ATOM | 1432 | O | ARG | A | 188 | 2.128 | 75.865 | 74.409 | 1.00 | 168.87 | 8 |
| ATOM | 1433 | N | THR | A | 189 | 0.750 | 77.649 | 74.338 | 1.00 | 187.44 | 7 |
| ATOM | 1434 | CA | THR | A | 189 | 1.259 | 78.160 | 73.069 | 1.00 | 184.73 | 6 |
| ATOM | 1435 | CB | THR | A | 189 | 0.939 | 79.662 | 72.903 | 1.00 | 190.85 | 6 |
| ATOM | 1436 | OG1 | THR | A | 189 | −0.479 | 79.859 | 72.975 | 1.00 | 191.29 | 8 |
| ATOM | 1437 | CG2 | THR | A | 189 | 1.445 | 80.172 | 71.559 | 1.00 | 190.83 | 6 |
| ATOM | 1438 | C | THR | A | 189 | 2.768 | 77.970 | 73.008 | 1.00 | 183.57 | 6 |
| ATOM | 1439 | O | THR | A | 189 | 3.528 | 78.815 | 73.482 | 1.00 | 183.68 | 8 |
| ATOM | 1440 | N | ASP | A | 190 | 3.191 | 76.852 | 72.424 | 1.00 | 96.77 | 7 |
| ATOM | 1441 | CA | ASP | A | 190 | 4.606 | 76.525 | 72.301 | 1.00 | 93.47 | 6 |
| ATOM | 1442 | CB | ASP | A | 190 | 5.046 | 75.638 | 73.464 | 1.00 | 145.73 | 6 |
| ATOM | 1443 | CG | ASP | A | 190 | 5.658 | 76.419 | 74.598 | 1.00 | 147.45 | 6 |
| ATOM | 1444 | OD1 | ASP | A | 190 | 6.643 | 77.144 | 74.345 | 1.00 | 148.52 | 8 |
| ATOM | 1445 | OD2 | ASP | A | 190 | 5.163 | 76.301 | 75.741 | 1.00 | 147.80 | 8 |
| ATOM | 1446 | C | ASP | A | 190 | 4.863 | 75.785 | 71.003 | 1.00 | 92.04 | 6 |
| ATOM | 1447 | O | ASP | A | 190 | 5.592 | 76.265 | 70.139 | 1.00 | 92.19 | 8 |
| ATOM | 1448 | N | LEU | A | 191 | 4.253 | 74.608 | 70.902 | 1.00 | 81.88 | 7 |
| ATOM | 1449 | CA | LEU | A | 191 | 4.351 | 73.704 | 69.753 | 1.00 | 78.93 | 6 |
| ATOM | 1450 | CB | LEU | A | 191 | 3.047 | 72.923 | 69.620 | 1.00 | 86.98 | 6 |
| ATOM | 1451 | CG | LEU | A | 191 | 2.224 | 72.796 | 70.898 | 1.00 | 87.57 | 6 |
| ATOM | 1452 | CD1 | LEU | A | 191 | 0.873 | 72.186 | 70.565 | 1.00 | 88.40 | 6 |
| ATOM | 1453 | CD2 | LEU | A | 191 | 2.981 | 71.956 | 71.911 | 1.00 | 88.35 | 6 |
| ATOM | 1454 | C | LEU | A | 191 | 4.661 | 74.334 | 68.391 | 1.00 | 76.05 | 6 |
| ATOM | 1455 | O | LEU | A | 191 | 4.416 | 75.519 | 68.158 | 1.00 | 75.94 | 8 |
| ATOM | 1456 | N | ASP | A | 192 | 5.198 | 73.520 | 67.487 | 1.00 | 94.98 | 7 |
| ATOM | 1457 | CA | ASP | A | 192 | 5.495 | 73.991 | 66.142 | 1.00 | 90.90 | 6 |
| ATOM | 1458 | CB | ASP | A | 192 | 6.811 | 73.386 | 65.580 | 1.00 | 64.72 | 6 |
| ATOM | 1459 | CG | ASP | A | 192 | 7.920 | 73.233 | 66.627 | 1.00 | 64.42 | 6 |
| ATOM | 1460 | OD1 | ASP | A | 192 | 8.246 | 74.208 | 67.333 | 1.00 | 65.46 | 8 |
| ATOM | 1461 | OD2 | ASP | A | 192 | 8.486 | 72.123 | 66.725 | 1.00 | 62.37 | 8 |
| ATOM | 1462 | C | ASP | A | 192 | 4.328 | 73.486 | 65.289 | 1.00 | 88.07 | 6 |
| ATOM | 1463 | O | ASP | A | 192 | 3.627 | 72.550 | 65.676 | 1.00 | 87.89 | 8 |
| ATOM | 1464 | N | LYS | A | 193 | 4.108 | 74.121 | 64.147 | 1.00 | 18.48 | 7 |
| ATOM | 1465 | CA | LYS | A | 193 | 3.075 | 73.685 | 63.230 | 1.00 | 13.87 | 6 |
| ATOM | 1466 | CB | LYS | A | 193 | 2.039 | 74.782 | 62.995 | 1.00 | 68.35 | 6 |
| ATOM | 1467 | CG | LYS | A | 193 | 1.084 | 74.454 | 61.853 | 1.00 | 70.13 | 6 |
| ATOM | 1468 | CD | LYS | A | 193 | 0.278 | 75.654 | 61.402 | 1.00 | 71.76 | 6 |
| ATOM | 1469 | CE | LYS | A | 193 | −0.453 | 75.341 | 60.102 | 1.00 | 72.69 | 6 |
| ATOM | 1470 | NZ | LYS | A | 193 | −1.416 | 76.411 | 59.712 | 1.00 | 74.05 | 7 |
| ATOM | 1471 | C | LYS | A | 193 | 3.772 | 73.356 | 61.905 | 1.00 | 13.87 | 6 |
| ATOM | 1472 | O | LYS | A | 193 | 3.733 | 74.134 | 60.954 | 1.00 | 13.87 | 8 |
| ATOM | 1473 | N | LEU | A | 194 | 4.411 | 72.194 | 61.837 | 1.00 | 49.50 | 7 |
| ATOM | 1474 | CA | LEU | A | 194 | 5.113 | 71.801 | 60.619 | 1.00 | 47.28 | 6 |
| ATOM | 1475 | CB | LEU | A | 194 | 5.700 | 70.408 | 60.779 | 1.00 | 40.96 | 6 |
| ATOM | 1476 | CG | LEU | A | 194 | 7.002 | 70.187 | 60.018 | 1.00 | 40.77 | 6 |
| ATOM | 1477 | CD1 | LEU | A | 194 | 7.432 | 68.770 | 60.271 | 1.00 | 42.47 | 6 |
| ATOM | 1478 | CD2 | LEU | A | 194 | 6.838 | 70.435 | 58.534 | 1.00 | 40.79 | 6 |
| ATOM | 1479 | C | LEU | A | 194 | 4.164 | 71.819 | 59.436 | 1.00 | 45.66 | 6 |
| ATOM | 1480 | O | LEU | A | 194 | 2.980 | 71.566 | 59.582 | 1.00 | 46.31 | 8 |
| ATOM | 1481 | N | THR | A | 195 | 4.673 | 72.115 | 58.255 | 1.00 | 23.66 | 7 |
| ATOM | 1482 | CA | THR | A | 195 | 3.795 | 72.156 | 57.112 | 1.00 | 23.58 | 6 |
| ATOM | 1483 | CB | THR | A | 195 | 2.764 | 73.333 | 57.272 | 1.00 | 47.51 | 6 |
| ATOM | 1484 | OG1 | THR | A | 195 | 2.457 | 73.916 | 55.999 | 1.00 | 49.59 | 8 |
| ATOM | 1485 | CG2 | THR | A | 195 | 3.307 | 74.398 | 58.183 | 1.00 | 49.19 | 6 |
| ATOM | 1486 | C | THR | A | 195 | 4.558 | 72.275 | 55.797 | 1.00 | 21.65 | 6 |
| ATOM | 1487 | O | THR | A | 195 | 4.413 | 73.270 | 55.077 | 1.00 | 20.96 | 8 |
| ATOM | 1488 | N | LEU | A | 196 | 5.359 | 71.260 | 55.459 | 1.00 | 13.87 | 7 |
| ATOM | 1489 | CA | LEU | A | 196 | 6.118 | 71.336 | 54.215 | 1.00 | 13.87 | 6 |
| ATOM | 1490 | CB | LEU | A | 196 | 7.198 | 70.268 | 54.118 | 1.00 | 13.87 | 6 |
| ATOM | 1491 | CG | LEU | A | 196 | 6.915 | 68.823 | 54.431 | 1.00 | 13.87 | 6 |
| ATOM | 1492 | CD1 | LEU | A | 196 | 8.258 | 68.110 | 54.425 | 1.00 | 13.87 | 6 |
| ATOM | 1493 | CD2 | LEU | A | 196 | 6.243 | 68.678 | 55.787 | 1.00 | 13.87 | 6 |
| ATOM | 1494 | C | LEU | A | 196 | 5.203 | 71.269 | 53.046 | 1.00 | 13.87 | 6 |
| ATOM | 1495 | O | LEU | A | 196 | 4.114 | 70.728 | 53.130 | 1.00 | 13.87 | 8 |
| ATOM | 1496 | N | ARG | A | 197 | 5.661 | 71.821 | 51.941 | 1.00 | 47.25 | 7 |
| ATOM | 1497 | CA | ARG | A | 197 | 4.821 | 71.899 | 50.775 | 1.00 | 51.20 | 6 |
| ATOM | 1498 | CB | ARG | A | 197 | 4.641 | 73.395 | 50.462 | 1.00 | 48.70 | 6 |
| ATOM | 1499 | CG | ARG | A | 197 | 4.293 | 74.237 | 51.717 | 1.00 | 48.34 | 6 |
| ATOM | 1500 | CD | ARG | A | 197 | 3.537 | 75.533 | 51.379 | 1.00 | 47.12 | 6 |
| ATOM | 1501 | NE | ARG | A | 197 | 4.399 | 76.676 | 51.091 | 1.00 | 46.27 | 7 |
| ATOM | 1502 | CZ | ARG | A | 197 | 3.991 | 77.799 | 50.504 | 1.00 | 46.69 | 6 |
| ATOM | 1503 | NH1 | ARG | A | 197 | 2.733 | 77.943 | 50.128 | 1.00 | 46.72 | 7 |
| ATOM | 1504 | NH2 | ARG | A | 197 | 4.842 | 78.793 | 50.303 | 1.00 | 46.95 | 7 |
| ATOM | 1505 | C | ARG | A | 197 | 5.201 | 71.099 | 49.512 | 1.00 | 52.55 | 6 |
| ATOM | 1506 | O | ARG | A | 197 | 4.357 | 70.908 | 48.627 | 1.00 | 52.73 | 8 |
| ATOM | 1507 | N | ILE | A | 198 | 6.433 | 70.595 | 49.435 | 1.00 | 26.26 | 7 |
| ATOM | 1508 | CA | ILE | A | 198 | 6.870 | 69.860 | 48.251 | 1.00 | 27.90 | 6 |
| ATOM | 1509 | CB | ILE | A | 198 | 6.540 | 68.363 | 48.304 | 1.00 | 33.44 | 6 |
| ATOM | 1510 | CG2 | ILE | A | 198 | 6.900 | 67.725 | 46.961 | 1.00 | 33.55 | 6 |
| ATOM | 1511 | CG1 | ILE | A | 198 | 7.342 | 67.658 | 49.403 | 1.00 | 33.58 | 6 |
| ATOM | 1512 | CD1 | ILE | A | 198 | 6.944 | 68.011 | 50.806 | 1.00 | 36.15 | 6 |
| ATOM | 1513 | C | ILE | A | 198 | 6.162 | 70.441 | 47.045 | 1.00 | 29.28 | 6 |
| ATOM | 1514 | O | ILE | A | 198 | 5.218 | 69.867 | 46.534 | 1.00 | 28.51 | 8 |
| ATOM | 1515 | N | TRP | A | 199 | 6.616 | 71.608 | 46.620 | 1.00 | 70.20 | 7 |
| ATOM | 1516 | CA | TRP | A | 199 | 6.053 | 72.300 | 45.476 | 1.00 | 74.84 | 6 |
| ATOM | 1517 | CB | TRP | A | 199 | 6.808 | 73.591 | 45.261 | 1.00 | 66.80 | 6 |
| ATOM | 1518 | CG | TRP | A | 199 | 5.977 | 74.760 | 44.961 | 1.00 | 67.13 | 6 |
| ATOM | 1519 | CD2 | TRP | A | 199 | 6.068 | 76.032 | 45.592 | 1.00 | 67.77 | 6 |
| ATOM | 1520 | CE2 | TRP | A | 199 | 5.173 | 76.891 | 44.927 | 1.00 | 68.77 | 6 |
| ATOM | 1521 | CE3 | TRP | A | 199 | 6.827 | 76.534 | 46.656 | 1.00 | 67.48 | 6 |
| ATOM | 1522 | CD1 | TRP | A | 199 | 5.051 | 74.881 | 43.971 | 1.00 | 67.47 | 6 |
| ATOM | 1523 | NE1 | TRP | A | 199 | 4.563 | 76.162 | 43.938 | 1.00 | 68.79 | 7 |
| ATOM | 1524 | CZ2 | TRP | A | 199 | 5.013 | 78.236 | 45.293 | 1.00 | 69.60 | 6 |
| ATOM | 1525 | CZ3 | TRP | A | 199 | 6.672 | 77.862 | 47.018 | 1.00 | 68.06 | 6 |
| ATOM | 1526 | CH2 | TRP | A | 199 | 5.773 | 78.700 | 46.339 | 1.00 | 69.07 | 6 |
| ATOM | 1527 | C | TRP | A | 199 | 6.181 | 71.445 | 44.222 | 1.00 | 77.11 | 6 |
| ATOM | 1528 | O | TRP | A | 199 | 6.307 | 70.227 | 44.299 | 1.00 | 78.95 | 8 |
| ATOM | 1529 | N | THR | A | 200 | 6.179 | 72.120 | 43.075 | 1.00 | 59.51 | 7 |
| ATOM | 1530 | CA | THR | A | 200 | 6.264 | 71.507 | 41.746 | 1.00 | 61.97 | 6 |
| ATOM | 1531 | CB | THR | A | 200 | 6.925 | 72.484 | 40.735 | 1.00 | 108.21 | 6 |
| ATOM | 1532 | OG1 | THR | A | 200 | 6.202 | 73.720 | 40.724 | 1.00 | 108.86 | 8 |
| ATOM | 1533 | CG2 | THR | A | 200 | 6.921 | 71.898 | 39.330 | 1.00 | 109.30 | 6 |
| ATOM | 1534 | C | THR | A | 200 | 6.990 | 70.163 | 41.638 | 1.00 | 61.64 | 6 |
| ATOM | 1535 | O | THR | A | 200 | 7.902 | 69.851 | 42.413 | 1.00 | 61.28 | 8 |
| ATOM | 1536 | N | ASP | A | 201 | 6.569 | 69.368 | 40.661 | 1.00 | 26.09 | 7 |
| ATOM | 1537 | CA | ASP | A | 201 | 7.185 | 68.082 | 40.413 | 1.00 | 27.98 | 6 |
| ATOM | 1538 | CB | ASP | A | 201 | 6.372 | 66.988 | 41.076 | 1.00 | 68.36 | 6 |
| ATOM | 1539 | CG | ASP | A | 201 | 6.389 | 67.104 | 42.566 | 1.00 | 71.72 | 6 |
| ATOM | 1540 | OD1 | ASP | A | 201 | 5.887 | 68.124 | 43.072 | 1.00 | 73.07 | 8 |
| ATOM | 1541 | OD2 | ASP | A | 201 | 6.903 | 66.181 | 43.232 | 1.00 | 73.40 | 8 |
| ATOM | 1542 | C | ASP | A | 201 | 7.372 | 67.772 | 38.922 | 1.00 | 29.33 | 6 |
| ATOM | 1543 | O | ASP | A | 201 | 6.637 | 68.285 | 38.057 | 1.00 | 29.41 | 8 |
| ATOM | 1544 | N | GLY | A | 202 | 8.379 | 66.952 | 38.621 | 1.00 | 97.53 | 7 |
| ATOM | 1545 | CA | GLY | A | 202 | 8.616 | 66.570 | 37.247 | 1.00 | 98.37 | 6 |
| ATOM | 1546 | C | GLY | A | 202 | 7.383 | 65.788 | 36.850 | 1.00 | 99.53 | 6 |
| ATOM | 1547 | O | GLY | A | 202 | 6.434 | 66.341 | 36.283 | 1.00 | 99.89 | 8 |
| ATOM | 1548 | N | SER | A | 203 | 7.382 | 64.501 | 37.187 | 1.00 | 102.96 | 7 |
| ATOM | 1549 | CA | SER | A | 203 | 6.258 | 63.625 | 36.882 | 1.00 | 101.80 | 6 |
| ATOM | 1550 | CB | SER | A | 203 | 6.750 | 62.365 | 36.168 | 1.00 | 86.92 | 6 |
| ATOM | 1551 | OG | SER | A | 203 | 7.569 | 61.575 | 37.014 | 1.00 | 84.76 | 8 |
| ATOM | 1552 | C | SER | A | 203 | 5.495 | 63.228 | 38.145 | 1.00 | 101.29 | 6 |
| ATOM | 1553 | O | SER | A | 203 | 4.282 | 63.427 | 38.219 | 1.00 | 101.42 | 8 |
| ATOM | 1554 | N | VAL | A | 204 | 6.212 | 62.687 | 39.133 | 1.00 | 55.39 | 7 |
| ATOM | 1555 | CA | VAL | A | 204 | 5.602 | 62.243 | 40.385 | 1.00 | 53.33 | 6 |
| ATOM | 1556 | CB | VAL | A | 204 | 6.618 | 61.599 | 41.316 | 1.00 | 24.32 | 6 |
| ATOM | 1557 | CG1 | VAL | A | 204 | 7.507 | 60.687 | 40.546 | 1.00 | 23.40 | 6 |
| ATOM | 1558 | CG2 | VAL | A | 204 | 7.383 | 62.654 | 42.044 | 1.00 | 23.67 | 6 |
| ATOM | 1559 | C | VAL | A | 204 | 4.858 | 63.288 | 41.210 | 1.00 | 52.68 | 6 |
| ATOM | 1560 | O | VAL | A | 204 | 4.943 | 64.493 | 40.965 | 1.00 | 52.37 | 8 |
| ATOM | 1561 | N | THR | A | 205 | 4.151 | 62.786 | 42.217 | 1.00 | 48.41 | 7 |
| ATOM | 1562 | CA | THR | A | 205 | 3.329 | 63.592 | 43.095 | 1.00 | 48.06 | 6 |
| ATOM | 1563 | CB | THR | A | 205 | 2.014 | 62.892 | 43.336 | 1.00 | 81.95 | 6 |
| ATOM | 1564 | OG1 | THR | A | 205 | 1.553 | 62.352 | 42.094 | 1.00 | 84.75 | 8 |
| ATOM | 1565 | CG2 | THR | A | 205 | 0.983 | 63.861 | 43.882 | 1.00 | 82.63 | 6 |
| ATOM | 1566 | C | THR | A | 205 | 3.979 | 63.857 | 44.434 | 1.00 | 46.17 | 6 |
| ATOM | 1567 | O | THR | A | 205 | 4.649 | 62.995 | 44.989 | 1.00 | 45.62 | 8 |
| ATOM | 1568 | N | PRO | A | 206 | 3.767 | 65.065 | 44.980 | 1.00 | 35.54 | 7 |
| ATOM | 1569 | CD | PRO | A | 206 | 3.005 | 66.193 | 44.404 | 1.00 | 15.27 | 6 |
| ATOM | 1570 | CA | PRO | A | 206 | 4.339 | 65.450 | 46.265 | 1.00 | 36.44 | 6 |
| ATOM | 1571 | CB | PRO | A | 206 | 3.639 | 66.763 | 46.551 | 1.00 | 14.81 | 6 |
| ATOM | 1572 | CG | PRO | A | 206 | 3.532 | 67.360 | 45.175 | 1.00 | 13.87 | 6 |
| ATOM | 1573 | C | PRO | A | 206 | 4.128 | 64.415 | 47.349 | 1.00 | 36.97 | 6 |
| ATOM | 1574 | O | PRO | A | 206 | 5.055 | 64.086 | 48.074 | 1.00 | 36.90 | 8 |
| ATOM | 1575 | N | LEU | A | 207 | 2.912 | 63.898 | 47.461 | 1.00 | 83.40 | 7 |
| ATOM | 1576 | CA | LEU | A | 207 | 2.622 | 62.891 | 48.473 | 1.00 | 85.60 | 6 |
| ATOM | 1577 | CB | LEU | A | 207 | 1.172 | 62.415 | 48.362 | 1.00 | 141.23 | 6 |
| ATOM | 1578 | CG | LEU | A | 207 | 0.790 | 61.243 | 49.272 | 1.00 | 141.75 | 6 |
| ATOM | 1579 | CD1 | LEU | A | 207 | 1.127 | 61.575 | 50.723 | 1.00 | 141.73 | 6 |
| ATOM | 1580 | CD2 | LEU | A | 207 | −0.690 | 60.937 | 49.107 | 1.00 | 142.22 | 6 |
| ATOM | 1581 | C | LEU | A | 207 | 3.566 | 61.712 | 48.284 | 1.00 | 85.87 | 6 |
| ATOM | 1582 | O | LEU | A | 207 | 3.850 | 60.975 | 49.225 | 1.00 | 86.19 | 8 |
| ATOM | 1583 | N | GLU | A | 208 | 4.046 | 61.542 | 47.055 | 1.00 | 68.68 | 7 |
| ATOM | 1584 | CA | GLU | A | 208 | 4.967 | 60.459 | 46.736 | 1.00 | 68.93 | 6 |
| ATOM | 1585 | CB | GLU | A | 208 | 4.865 | 60.109 | 45.242 | 1.00 | 99.32 | 6 |
| ATOM | 1586 | CG | GLU | A | 208 | 3.421 | 59.967 | 44.737 | 1.00 | 99.03 | 6 |
| ATOM | 1587 | CD | GLU | A | 208 | 3.320 | 59.389 | 43.328 | 1.00 | 99.46 | 6 |
| ATOM | 1588 | OE1 | GLU | A | 208 | 3.687 | 58.209 | 43.142 | 1.00 | 99.84 | 8 |
| ATOM | 1589 | OE2 | GLU | A | 208 | 2.872 | 60.111 | 42.406 | 1.00 | 99.14 | 8 |
| ATOM | 1590 | C | GLU | A | 208 | 6.378 | 60.936 | 47.103 | 1.00 | 68.54 | 6 |
| ATOM | 1591 | O | GLU | A | 208 | 7.150 | 60.215 | 47.741 | 1.00 | 67.68 | 8 |
| ATOM | 1592 | N | ALA | A | 209 | 6.710 | 62.159 | 46.701 | 1.00 | 75.76 | 7 |
| ATOM | 1593 | CA | ALA | A | 209 | 8.007 | 62.720 | 47.038 | 1.00 | 75.57 | 6 |
| ATOM | 1594 | CB | ALA | A | 209 | 8.098 | 64.190 | 46.570 | 1.00 | 13.87 | 6 |
| ATOM | 1595 | C | ALA | A | 209 | 8.041 | 62.631 | 48.560 | 1.00 | 75.45 | 6 |
| ATOM | 1596 | O | ALA | A | 209 | 8.779 | 61.831 | 49.128 | 1.00 | 75.66 | 8 |
| ATOM | 1597 | N | LEU | A | 210 | 7.204 | 63.438 | 49.205 | 1.00 | 70.26 | 7 |
| ATOM | 1598 | CA | LEU | A | 210 | 7.089 | 63.467 | 50.658 | 1.00 | 69.79 | 6 |
| ATOM | 1599 | CB | LEU | A | 210 | 5.782 | 64.151 | 51.057 | 1.00 | 48.86 | 6 |
| ATOM | 1600 | CG | LEU | A | 210 | 5.176 | 63.720 | 52.392 | 1.00 | 48.50 | 6 |
| ATOM | 1601 | CD1 | LEU | A | 210 | 6.079 | 64.139 | 53.540 | 1.00 | 48.16 | 6 |
| ATOM | 1602 | CD2 | LEU | A | 210 | 3.789 | 64.321 | 52.526 | 1.00 | 49.32 | 6 |
| ATOM | 1603 | C | LEU | A | 210 | 7.133 | 62.071 | 51.277 | 1.00 | 69.96 | 6 |
| ATOM | 1604 | O | LEU | A | 210 | 7.604 | 61.901 | 52.407 | 1.00 | 69.73 | 8 |
| ATOM | 1605 | N | ASN | A | 211 | 6.627 | 61.080 | 50.544 | 1.00 | 88.32 | 7 |
| ATOM | 1606 | CA | ASN | A | 211 | 6.612 | 59.699 | 51.031 | 1.00 | 88.59 | 6 |
| ATOM | 1607 | CB | ASN | A | 211 | 5.539 | 58.874 | 50.302 | 1.00 | 54.35 | 6 |
| ATOM | 1608 | CG | ASN | A | 211 | 4.257 | 58.734 | 51.111 | 1.00 | 53.18 | 6 |
| ATOM | 1609 | OD1 | ASN | A | 211 | 3.169 | 59.092 | 50.650 | 1.00 | 51.67 | 8 |
| ATOM | 1610 | ND2 | ASN | A | 211 | 4.384 | 58.209 | 52.330 | 1.00 | 53.78 | 7 |
| ATOM | 1611 | C | ASN | A | 211 | 7.974 | 59.063 | 50.826 | 1.00 | 88.51 | 6 |
| ATOM | 1612 | O | ASN | A | 211 | 8.679 | 58.750 | 51.785 | 1.00 | 88.15 | 8 |
| ATOM | 1613 | N | GLN | A | 212 | 8.335 | 58.877 | 49.563 | 1.00 | 102.24 | 7 |
| ATOM | 1614 | CA | GLN | A | 212 | 9.617 | 58.296 | 49.202 | 1.00 | 102.01 | 6 |
| ATOM | 1615 | CB | GLN | A | 212 | 9.923 | 58.634 | 47.745 | 1.00 | 132.93 | 6 |
| ATOM | 1616 | CG | GLN | A | 212 | 10.206 | 57.438 | 46.870 | 1.00 | 135.91 | 6 |
| ATOM | 1617 | CD | GLN | A | 212 | 9.778 | 57.674 | 45.438 | 1.00 | 138.12 | 6 |
| ATOM | 1618 | OE1 | GLN | A | 212 | 10.162 | 58.665 | 44.816 | 1.00 | 138.09 | 8 |
| ATOM | 1619 | NE2 | GLN | A | 212 | 8.976 | 56.762 | 44.904 | 1.00 | 139.89 | 7 |
| ATOM | 1620 | C | GLN | A | 212 | 10.666 | 58.910 | 50.118 | 1.00 | 100.76 | 6 |
| ATOM | 1621 | O | GLN | A | 212 | 11.576 | 58.234 | 50.592 | 1.00 | 100.37 | 8 |
| ATOM | 1622 | N | ALA | A | 213 | 10.507 | 60.204 | 50.373 | 1.00 | 67.79 | 7 |
| ATOM | 1623 | CA | ALA | A | 213 | 11.420 | 60.940 | 51.220 | 1.00 | 65.50 | 6 |
| ATOM | 1624 | CB | ALA | A | 213 | 10.995 | 62.382 | 51.298 | 1.00 | 13.87 | 6 |
| ATOM | 1625 | C | ALA | A | 213 | 11.475 | 60.333 | 52.608 | 1.00 | 64.69 | 6 |
| ATOM | 1626 | O | ALA | A | 213 | 12.525 | 59.856 | 53.030 | 1.00 | 65.22 | 8 |
| ATOM | 1627 | N | VAL | A | 214 | 10.349 | 60.341 | 53.317 | 1.00 | 68.52 | 7 |
| ATOM | 1628 | CA | VAL | A | 214 | 10.311 | 59.790 | 54.673 | 1.00 | 69.21 | 6 |
| ATOM | 1629 | CB | VAL | A | 214 | 8.852 | 59.699 | 55.228 | 1.00 | 100.42 | 6 |
| ATOM | 1630 | CG1 | VAL | A | 214 | 8.155 | 61.040 | 55.090 | 1.00 | 101.75 | 6 |
| ATOM | 1631 | CG2 | VAL | A | 214 | 8.069 | 58.622 | 54.503 | 1.00 | 101.44 | 6 |
| ATOM | 1632 | C | VAL | A | 214 | 10.934 | 58.397 | 54.694 | 1.00 | 68.93 | 6 |
| ATOM | 1633 | O | VAL | A | 214 | 11.367 | 57.906 | 55.738 | 1.00 | 69.47 | 8 |
| ATOM | 1634 | N | ALA | A | 215 | 10.986 | 57.777 | 53.519 | 1.00 | 54.30 | 7 |
| ATOM | 1635 | CA | ALA | A | 215 | 11.531 | 56.438 | 53.371 | 1.00 | 51.91 | 6 |
| ATOM | 1636 | CB | ALA | A | 215 | 11.193 | 55.893 | 51.997 | 1.00 | 65.24 | 6 |
| ATOM | 1637 | C | ALA | A | 215 | 13.023 | 56.442 | 53.567 | 1.00 | 50.60 | 6 |
| ATOM | 1638 | O | ALA | A | 215 | 13.527 | 55.844 | 54.507 | 1.00 | 50.82 | 8 |
| ATOM | 1639 | N | ILE | A | 216 | 13.721 | 57.129 | 52.671 | 1.00 | 43.01 | 7 |
| ATOM | 1640 | CA | ILE | A | 216 | 15.172 | 57.198 | 52.722 | 1.00 | 41.44 | 6 |
| ATOM | 1641 | CB | ILE | A | 216 | 15.730 | 58.209 | 51.708 | 1.00 | 19.95 | 6 |
| ATOM | 1642 | CG2 | ILE | A | 216 | 17.099 | 57.774 | 51.249 | 1.00 | 18.66 | 6 |
| ATOM | 1643 | CG1 | ILE | A | 216 | 14.829 | 58.282 | 50.484 | 1.00 | 19.48 | 6 |
| ATOM | 1644 | CD1 | ILE | A | 216 | 14.631 | 56.966 | 49.787 | 1.00 | 21.24 | 6 |
| ATOM | 1645 | C | ILE | A | 216 | 15.663 | 57.587 | 54.108 | 1.00 | 41.48 | 6 |
| ATOM | 1646 | O | ILE | A | 216 | 16.557 | 56.940 | 54.656 | 1.00 | 41.44 | 8 |
| ATOM | 1647 | N | LEU | A | 217 | 15.072 | 58.635 | 54.679 | 1.00 | 28.89 | 7 |
| ATOM | 1648 | CA | LEU | A | 217 | 15.470 | 59.104 | 56.003 | 1.00 | 28.88 | 6 |
| ATOM | 1649 | CB | LEU | A | 217 | 14.667 | 60.346 | 56.379 | 1.00 | 22.95 | 6 |
| ATOM | 1650 | CG | LEU | A | 217 | 14.900 | 60.934 | 57.770 | 1.00 | 21.45 | 6 |
| ATOM | 1651 | CD1 | LEU | A | 217 | 16.368 | 60.994 | 58.127 | 1.00 | 20.21 | 6 |
| ATOM | 1652 | CD2 | LEU | A | 217 | 14.311 | 62.321 | 57.757 | 1.00 | 21.23 | 6 |
| ATOM | 1653 | C | LEU | A | 217 | 15.320 | 58.037 | 57.082 | 1.00 | 29.41 | 6 |
| ATOM | 1654 | O | LEU | A | 217 | 16.083 | 58.010 | 58.049 | 1.00 | 29.86 | 8 |
| ATOM | 1655 | N | LYS | A | 218 | 14.328 | 57.164 | 56.913 | 1.00 | 50.57 | 7 |
| ATOM | 1656 | CA | LYS | A | 218 | 14.084 | 56.078 | 57.859 | 1.00 | 51.86 | 6 |
| ATOM | 1657 | CB | LYS | A | 218 | 12.685 | 55.496 | 57.679 | 1.00 | 57.70 | 6 |
| ATOM | 1658 | CG | LYS | A | 218 | 11.535 | 56.416 | 58.036 | 1.00 | 59.45 | 6 |
| ATOM | 1659 | CD | LYS | A | 218 | 10.227 | 55.649 | 57.956 | 1.00 | 61.30 | 6 |
| ATOM | 1660 | CE | LYS | A | 218 | 9.056 | 56.466 | 58.459 | 1.00 | 63.03 | 6 |
| ATOM | 1661 | NZ | LYS | A | 218 | 7.845 | 55.607 | 58.644 | 1.00 | 64.61 | 7 |
| ATOM | 1662 | C | LYS | A | 218 | 15.095 | 54.993 | 57.567 | 1.00 | 51.99 | 6 |
| ATOM | 1663 | O | LYS | A | 218 | 15.618 | 54.363 | 58.477 | 1.00 | 51.13 | 8 |
| ATOM | 1664 | N | GLU | A | 219 | 15.345 | 54.792 | 56.275 | 1.00 | 59.27 | 7 |
| ATOM | 1665 | CA | GLU | A | 219 | 16.283 | 53.799 | 55.766 | 1.00 | 61.46 | 6 |
| ATOM | 1666 | CB | GLU | A | 219 | 16.251 | 53.828 | 54.233 | 1.00 | 113.88 | 6 |
| ATOM | 1667 | CG | GLU | A | 219 | 16.891 | 52.640 | 53.528 | 1.00 | 117.59 | 6 |
| ATOM | 1668 | CD | GLU | A | 219 | 16.680 | 52.676 | 52.011 | 1.00 | 120.81 | 6 |
| ATOM | 1669 | OE1 | GLU | A | 219 | 15.515 | 52.611 | 51.550 | 1.00 | 121.78 | 8 |
| ATOM | 1670 | OE2 | GLU | A | 219 | 17.685 | 52.770 | 51.273 | 1.00 | 122.09 | 8 |
| ATOM | 1671 | C | GLU | A | 219 | 17.689 | 54.110 | 56.285 | 1.00 | 60.81 | 6 |
| ATOM | 1672 | O | GLU | A | 219 | 18.427 | 53.215 | 56.706 | 1.00 | 60.38 | 8 |
| ATOM | 1673 | N | HIS | A | 220 | 18.054 | 55.386 | 56.262 | 1.00 | 65.87 | 7 |
| ATOM | 1674 | CA | HIS | A | 220 | 19.359 | 55.800 | 56.741 | 1.00 | 65.62 | 6 |
| ATOM | 1675 | CB | HIS | A | 220 | 19.667 | 57.206 | 56.282 | 1.00 | 39.54 | 6 |
| ATOM | 1676 | CG | HIS | A | 220 | 19.896 | 57.301 | 54.816 | 1.00 | 38.27 | 6 |
| ATOM | 1677 | CD2 | HIS | A | 220 | 19.095 | 57.728 | 53.815 | 1.00 | 37.65 | 6 |
| ATOM | 1678 | ND1 | HIS | A | 220 | 21.047 | 56.838 | 54.218 | 1.00 | 37.62 | 7 |
| ATOM | 1679 | CE1 | HIS | A | 220 | 20.942 | 56.973 | 52.909 | 1.00 | 37.73 | 6 |
| ATOM | 1680 | NE2 | HIS | A | 220 | 19.766 | 57.510 | 52.639 | 1.00 | 37.89 | 7 |
| ATOM | 1681 | C | HIS | A | 220 | 19.377 | 55.752 | 58.237 | 1.00 | 65.89 | 6 |
| ATOM | 1682 | O | HIS | A | 220 | 20.261 | 55.148 | 58.828 | 1.00 | 67.49 | 8 |
| ATOM | 1683 | N | LEU | A | 221 | 18.396 | 56.397 | 58.853 | 1.00 | 48.36 | 7 |
| ATOM | 1684 | CA | LEU | A | 221 | 18.305 | 56.408 | 60.304 | 1.00 | 48.22 | 6 |
| ATOM | 1685 | CB | LEU | A | 221 | 16.867 | 56.693 | 60.721 | 1.00 | 31.54 | 6 |
| ATOM | 1686 | CG | LEU | A | 221 | 16.566 | 57.975 | 61.495 | 1.00 | 30.44 | 6 |
| ATOM | 1687 | CD1 | LEU | A | 221 | 17.607 | 58.127 | 62.603 | 1.00 | 30.22 | 6 |
| ATOM | 1688 | CD2 | LEU | A | 221 | 16.553 | 59.181 | 60.566 | 1.00 | 29.38 | 6 |
| ATOM | 1689 | C | LEU | A | 221 | 18.736 | 55.047 | 60.862 | 1.00 | 48.73 | 6 |
| ATOM | 1690 | O | LEU | A | 221 | 19.479 | 54.960 | 61.849 | 1.00 | 48.06 | 8 |
| ATOM | 1691 | N | ASN | A | 222 | 18.267 | 53.990 | 60.199 | 1.00 | 57.57 | 7 |
| ATOM | 1692 | CA | ASN | A | 222 | 18.561 | 52.618 | 60.594 | 1.00 | 58.23 | 6 |
| ATOM | 1693 | CB | ASN | A | 222 | 18.084 | 51.638 | 59.523 | 1.00 | 62.29 | 6 |
| ATOM | 1694 | CG | ASN | A | 222 | 16.568 | 51.594 | 59.405 | 1.00 | 62.92 | 6 |
| ATOM | 1695 | OD1 | ASN | A | 222 | 15.856 | 51.358 | 60.386 | 1.00 | 63.28 | 8 |
| ATOM | 1696 | ND2 | ASN | A | 222 | 16.067 | 51.816 | 58.199 | 1.00 | 63.76 | 7 |
| ATOM | 1697 | C | ASN | A | 222 | 20.032 | 52.423 | 60.828 | 1.00 | 58.36 | 6 |
| ATOM | 1698 | O | ASN | A | 222 | 20.447 | 52.071 | 61.926 | 1.00 | 58.19 | 8 |
| ATOM | 1699 | N | TYR | A | 223 | 20.810 | 52.667 | 59.782 | 1.00 | 57.73 | 7 |
| ATOM | 1700 | CA | TYR | A | 223 | 22.261 | 52.520 | 59.819 | 1.00 | 58.14 | 6 |
| ATOM | 1701 | CB | TYR | A | 223 | 22.914 | 53.522 | 58.857 | 1.00 | 81.51 | 6 |
| ATOM | 1702 | CG | TYR | A | 223 | 22.622 | 53.233 | 57.395 | 1.00 | 81.37 | 6 |
| ATOM | 1703 | CD1 | TYR | A | 223 | 22.759 | 51.944 | 56.883 | 1.00 | 81.65 | 6 |
| ATOM | 1704 | CE1 | TYR | A | 223 | 22.503 | 51.670 | 55.549 | 1.00 | 82.13 | 6 |
| ATOM | 1705 | CD2 | TYR | A | 223 | 22.221 | 54.245 | 56.525 | 1.00 | 80.64 | 6 |
| ATOM | 1706 | CE2 | TYR | A | 223 | 21.963 | 53.980 | 55.189 | 1.00 | 81.03 | 6 |
| ATOM | 1707 | CZ | TYR | A | 223 | 22.105 | 52.691 | 54.708 | 1.00 | 81.92 | 6 |
| ATOM | 1708 | OH | TYR | A | 223 | 21.842 | 52.412 | 53.386 | 1.00 | 82.77 | 8 |
| ATOM | 1709 | C | TYR | A | 223 | 22.911 | 52.622 | 61.197 | 1.00 | 58.19 | 6 |
| ATOM | 1710 | O | TYR | A | 223 | 23.891 | 51.921 | 61.467 | 1.00 | 58.31 | 8 |
| ATOM | 1711 | N | PHE | A | 224 | 22.388 | 53.482 | 62.067 | 1.00 | 62.07 | 7 |
| ATOM | 1712 | CA | PHE | A | 224 | 22.964 | 53.592 | 63.403 | 1.00 | 63.51 | 6 |
| ATOM | 1713 | CB | PHE | A | 224 | 22.478 | 54.865 | 64.119 | 1.00 | 39.72 | 6 |
| ATOM | 1714 | CG | PHE | A | 224 | 22.643 | 56.148 | 63.319 | 1.00 | 36.56 | 6 |
| ATOM | 1715 | CD1 | PHE | A | 224 | 21.870 | 56.389 | 62.183 | 1.00 | 35.76 | 6 |
| ATOM | 1716 | CD2 | PHE | A | 224 | 23.518 | 57.139 | 63.743 | 1.00 | 34.63 | 6 |
| ATOM | 1717 | CE1 | PHE | A | 224 | 21.967 | 57.602 | 61.490 | 1.00 | 34.55 | 6 |
| ATOM | 1718 | CE2 | PHE | A | 224 | 23.616 | 58.340 | 63.057 | 1.00 | 34.38 | 6 |
| ATOM | 1719 | CZ | PHE | A | 224 | 22.837 | 58.573 | 61.929 | 1.00 | 33.84 | 6 |
| ATOM | 1720 | C | PHE | A | 224 | 22.505 | 52.342 | 64.177 | 1.00 | 64.88 | 6 |
| ATOM | 1721 | O | PHE | A | 224 | 21.931 | 52.437 | 65.266 | 1.00 | 64.71 | 8 |
| ATOM | 1722 | N | ALA | A | 225 | 22.773 | 51.173 | 63.598 | 1.00 | 57.96 | 7 |
| ATOM | 1723 | CA | ALA | A | 225 | 22.370 | 49.899 | 64.179 | 1.00 | 60.31 | 6 |
| ATOM | 1724 | CB | ALA | A | 225 | 21.893 | 48.968 | 63.074 | 1.00 | 89.79 | 6 |
| ATOM | 1725 | C | ALA | A | 225 | 23.424 | 49.192 | 65.022 | 1.00 | 61.98 | 6 |
| ATOM | 1726 | O | ALA | A | 225 | 23.407 | 49.299 | 66.244 | 1.00 | 62.94 | 8 |
| ATOM | 1727 | N | ASN | A | 226 | 24.324 | 48.451 | 64.376 | 1.00 | 79.27 | 7 |
| ATOM | 1728 | CA | ASN | A | 226 | 25.369 | 47.711 | 65.092 | 1.00 | 80.93 | 6 |
| ATOM | 1729 | CB | ASN | A | 226 | 25.399 | 46.255 | 64.621 | 1.00 | 184.67 | 6 |
| ATOM | 1730 | CG | ASN | A | 226 | 24.239 | 45.444 | 65.168 | 1.00 | 186.86 | 6 |
| ATOM | 1731 | OD1 | ASN | A | 226 | 24.046 | 44.287 | 64.793 | 1.00 | 187.92 | 8 |
| ATOM | 1732 | ND2 | ASN | A | 226 | 23.463 | 46.045 | 66.066 | 1.00 | 187.67 | 7 |
| ATOM | 1733 | C | ASN | A | 226 | 26.759 | 48.329 | 64.969 | 1.00 | 80.54 | 6 |
| ATOM | 1734 | O | ASN | A | 226 | 27.355 | 48.365 | 63.892 | 1.00 | 80.25 | 8 |
| ATOM | 1735 | N | PRO | A | 227 | 27.301 | 48.806 | 66.095 | 1.00 | 31.83 | 7 |
| ATOM | 1736 | CD | PRO | A | 227 | 26.576 | 48.977 | 67.370 | 1.00 | 64.99 | 6 |
| ATOM | 1737 | CA | PRO | A | 227 | 28.614 | 49.440 | 66.160 | 1.00 | 32.33 | 6 |
| ATOM | 1738 | CB | PRO | A | 227 | 28.393 | 50.513 | 67.207 | 1.00 | 64.99 | 6 |
| ATOM | 1739 | CG | PRO | A | 227 | 27.572 | 49.753 | 68.225 | 1.00 | 64.42 | 6 |
| ATOM | 1740 | C | PRO | A | 227 | 29.736 | 48.501 | 66.549 | 1.00 | 33.14 | 6 |
| ATOM | 1741 | O | PRO | A | 227 | 29.510 | 47.554 | 67.297 | 1.00 | 32.56 | 8 |
| ATOM | 1742 | N | GLU | A | 228 | 30.944 | 48.779 | 66.054 | 1.00 | 45.58 | 7 |
| ATOM | 1743 | CA | GLU | A | 228 | 32.119 | 47.962 | 66.368 | 1.00 | 47.66 | 6 |
| ATOM | 1744 | CB | GLU | A | 228 | 32.962 | 47.714 | 65.112 | 1.00 | 122.21 | 6 |
| ATOM | 1745 | CG | GLU | A | 228 | 32.226 | 47.022 | 63.975 | 1.00 | 125.27 | 6 |
| ATOM | 1746 | CD | GLU | A | 228 | 33.048 | 46.960 | 62.698 | 1.00 | 126.99 | 6 |
| ATOM | 1747 | OE1 | GLU | A | 228 | 33.469 | 48.027 | 62.204 | 1.00 | 127.85 | 8 |
| ATOM | 1748 | OE2 | GLU | A | 228 | 33.272 | 45.846 | 62.184 | 1.00 | 128.27 | 8 |
| ATOM | 1749 | C | GLU | A | 228 | 32.961 | 48.719 | 67.387 | 1.00 | 47.81 | 6 |
| ATOM | 1750 | O | GLU | A | 228 | 32.563 | 48.761 | 68.572 | 1.00 | 48.48 | 8 |
| ATOM | 1751 | OXT | GLU | A | 228 | 34.003 | 49.278 | 66.984 | 1.00 | 122.18 | 8 |
| ATOM | 1752 | CB | ALA | B | 3 | 36.891 | 46.742 | 48.658 | 1.00 | 177.12 | 6 |
| ATOM | 1753 | C | ALA | B | 3 | 35.150 | 46.906 | 50.443 | 1.00 | 140.19 | 6 |
| ATOM | 1754 | O | ALA | B | 3 | 35.794 | 46.246 | 51.264 | 1.00 | 140.05 | 8 |
| ATOM | 1755 | N | ALA | B | 3 | 34.714 | 47.787 | 48.149 | 1.00 | 139.84 | 7 |
| ATOM | 1756 | CA | ALA | B | 3 | 35.397 | 46.724 | 48.947 | 1.00 | 139.99 | 6 |
| ATOM | 1757 | N | SER | B | 4 | 34.224 | 47.811 | 50.775 | 1.00 | 101.63 | 7 |
| ATOM | 1758 | CA | SER | B | 4 | 33.825 | 48.123 | 52.156 | 1.00 | 100.90 | 6 |
| ATOM | 1759 | CB | SER | B | 4 | 34.990 | 47.939 | 53.138 | 1.00 | 153.10 | 6 |
| ATOM | 1760 | OG | SER | B | 4 | 35.096 | 46.587 | 53.558 | 1.00 | 154.08 | 8 |
| ATOM | 1761 | C | SER | B | 4 | 33.278 | 49.539 | 52.305 | 1.00 | 99.84 | 6 |
| ATOM | 1762 | O | SER | B | 4 | 34.037 | 50.510 | 52.324 | 1.00 | 100.06 | 8 |
| ATOM | 1763 | N | ALA | B | 5 | 31.956 | 49.630 | 52.423 | 1.00 | 120.11 | 7 |
| ATOM | 1764 | CA | ALA | B | 5 | 31.232 | 50.889 | 52.586 | 1.00 | 119.35 | 6 |
| ATOM | 1765 | CB | ALA | B | 5 | 31.983 | 52.033 | 51.925 | 1.00 | 90.01 | 6 |
| ATOM | 1766 | C | ALA | B | 5 | 29.853 | 50.744 | 51.955 | 1.00 | 118.60 | 6 |
| ATOM | 1767 | O | ALA | B | 5 | 28.836 | 50.697 | 52.649 | 1.00 | 118.65 | 8 |
| ATOM | 1768 | N | ALA | B | 6 | 29.845 | 50.667 | 50.627 | 1.00 | 101.80 | 7 |
| ATOM | 1769 | CA | ALA | B | 6 | 28.631 | 50.519 | 49.829 | 1.00 | 101.01 | 6 |
| ATOM | 1770 | CB | ALA | B | 6 | 28.490 | 49.062 | 49.369 | 1.00 | 18.92 | 6 |
| ATOM | 1771 | C | ALA | B | 6 | 27.351 | 50.984 | 50.526 | 1.00 | 100.19 | 6 |
| ATOM | 1772 | O | ALA | B | 6 | 27.349 | 51.981 | 51.249 | 1.00 | 101.16 | 8 |
| ATOM | 1773 | N | LYS | B | 7 | 26.261 | 50.260 | 50.293 | 1.00 | 113.51 | 7 |
| ATOM | 1774 | CA | LYS | B | 7 | 24.971 | 50.601 | 50.878 | 1.00 | 111.85 | 6 |
| ATOM | 1775 | CB | LYS | B | 7 | 23.841 | 50.078 | 49.985 | 1.00 | 206.39 | 6 |
| ATOM | 1776 | CG | LYS | B | 7 | 23.851 | 50.697 | 48.595 | 1.00 | 208.87 | 6 |
| ATOM | 1777 | CD | LYS | B | 7 | 22.788 | 50.116 | 47.670 | 1.00 | 208.87 | 6 |
| ATOM | 1778 | CE | LYS | B | 7 | 22.858 | 50.783 | 46.293 | 1.00 | 208.87 | 6 |
| ATOM | 1779 | NZ | LYS | B | 7 | 21.902 | 50.212 | 45.298 | 1.00 | 208.87 | 7 |
| ATOM | 1780 | C | LYS | B | 7 | 24.821 | 50.048 | 52.285 | 1.00 | 109.93 | 6 |
| ATOM | 1781 | O | LYS | B | 7 | 23.808 | 49.435 | 52.615 | 1.00 | 110.05 | 8 |
| ATOM | 1782 | N | ALA | B | 8 | 25.835 | 50.272 | 53.114 | 1.00 | 61.60 | 7 |
| ATOM | 1783 | CA | ALA | B | 8 | 25.815 | 49.803 | 54.492 | 1.00 | 58.50 | 6 |
| ATOM | 1784 | CB | ALA | B | 8 | 25.895 | 48.298 | 54.521 | 1.00 | 56.84 | 6 |
| ATOM | 1785 | C | ALA | B | 8 | 26.989 | 50.402 | 55.254 | 1.00 | 56.66 | 6 |
| ATOM | 1786 | O | ALA | B | 8 | 28.016 | 49.749 | 55.439 | 1.00 | 56.49 | 8 |
| ATOM | 1787 | N | PRO | B | 9 | 26.847 | 51.654 | 55.717 | 1.00 | 53.70 | 7 |
| ATOM | 1788 | CD | PRO | B | 9 | 25.625 | 52.472 | 55.654 | 1.00 | 64.50 | 6 |
| ATOM | 1789 | CA | PRO | B | 9 | 27.898 | 52.354 | 56.460 | 1.00 | 51.92 | 6 |
| ATOM | 1790 | CB | PRO | B | 9 | 27.319 | 53.753 | 56.635 | 1.00 | 63.49 | 6 |
| ATOM | 1791 | CG | PRO | B | 9 | 25.857 | 53.478 | 56.754 | 1.00 | 64.27 | 6 |
| ATOM | 1792 | C | PRO | B | 9 | 28.239 | 51.697 | 57.784 | 1.00 | 51.30 | 6 |
| ATOM | 1793 | O | PRO | B | 9 | 27.392 | 51.053 | 58.400 | 1.00 | 51.69 | 8 |
| ATOM | 1794 | N | VAL | B | 10 | 29.487 | 51.878 | 58.207 | 1.00 | 51.49 | 7 |
| ATOM | 1795 | CA | VAL | B | 10 | 29.983 | 51.312 | 59.450 | 1.00 | 50.52 | 6 |
| ATOM | 1796 | CB | VAL | B | 10 | 31.460 | 50.948 | 59.346 | 1.00 | 58.34 | 6 |
| ATOM | 1797 | CG1 | VAL | B | 10 | 31.892 | 50.206 | 60.596 | 1.00 | 58.83 | 6 |
| ATOM | 1798 | CG2 | VAL | B | 10 | 31.705 | 50.121 | 58.100 | 1.00 | 58.45 | 6 |
| ATOM | 1799 | C | VAL | B | 10 | 29.846 | 52.297 | 60.590 | 1.00 | 50.72 | 6 |
| ATOM | 1800 | O | VAL | B | 10 | 30.563 | 53.298 | 60.636 | 1.00 | 51.63 | 8 |
| ATOM | 1801 | N | PHE | B | 11 | 28.929 | 51.986 | 61.505 | 1.00 | 53.80 | 7 |
| ATOM | 1802 | CA | PHE | B | 11 | 28.627 | 52.789 | 62.693 | 1.00 | 52.66 | 6 |
| ATOM | 1803 | CB | PHE | B | 11 | 27.231 | 52.403 | 63.167 | 1.00 | 77.05 | 6 |
| ATOM | 1804 | CG | PHE | B | 11 | 26.651 | 53.330 | 64.173 | 1.00 | 77.97 | 6 |
| ATOM | 1805 | CD1 | PHE | B | 11 | 26.463 | 54.671 | 63.869 | 1.00 | 78.45 | 6 |
| ATOM | 1806 | CD2 | PHE | B | 11 | 26.251 | 52.855 | 65.418 | 1.00 | 79.06 | 6 |
| ATOM | 1807 | CE1 | PHE | B | 11 | 25.883 | 55.527 | 64.786 | 1.00 | 78.89 | 6 |
| ATOM | 1808 | CE2 | PHE | B | 11 | 25.667 | 53.704 | 66.346 | 1.00 | 79.40 | 6 |
| ATOM | 1809 | CZ | PHE | B | 11 | 25.482 | 55.044 | 66.029 | 1.00 | 79.32 | 6 |
| ATOM | 1810 | C | PHE | B | 11 | 29.673 | 52.486 | 63.786 | 1.00 | 53.13 | 6 |
| ATOM | 1811 | O | PHE | B | 11 | 29.660 | 51.407 | 64.379 | 1.00 | 53.82 | 8 |
| ATOM | 1812 | N | THR | B | 12 | 30.575 | 53.429 | 64.056 | 1.00 | 66.78 | 7 |
| ATOM | 1813 | CA | THR | B | 12 | 31.636 | 53.207 | 65.047 | 1.00 | 67.68 | 6 |
| ATOM | 1814 | CB | THR | B | 12 | 33.026 | 53.500 | 64.437 | 1.00 | 114.71 | 6 |
| ATOM | 1815 | OG1 | THR | B | 12 | 33.244 | 52.650 | 63.305 | 1.00 | 115.14 | 8 |
| ATOM | 1816 | CG2 | THR | B | 12 | 34.120 | 53.252 | 65.460 | 1.00 | 115.46 | 6 |
| ATOM | 1817 | C | THR | B | 12 | 31.501 | 54.033 | 66.323 | 1.00 | 68.21 | 6 |
| ATOM | 1818 | O | THR | B | 12 | 31.599 | 55.258 | 66.286 | 1.00 | 68.13 | 8 |
| ATOM | 1819 | N | ALA | B | 13 | 31.315 | 53.351 | 67.454 | 1.00 | 70.17 | 7 |
| ATOM | 1820 | CA | ALA | B | 13 | 31.151 | 54.002 | 68.760 | 1.00 | 71.26 | 6 |
| ATOM | 1821 | CB | ALA | B | 13 | 30.227 | 53.173 | 69.630 | 1.00 | 41.04 | 6 |
| ATOM | 1822 | C | ALA | B | 13 | 32.459 | 54.233 | 69.502 | 1.00 | 73.13 | 6 |
| ATOM | 1823 | O | ALA | B | 13 | 33.526 | 53.838 | 69.038 | 1.00 | 74.07 | 8 |
| ATOM | 1824 | N | THR | B | 14 | 32.371 | 54.867 | 70.668 | 1.00 | 89.91 | 7 |
| ATOM | 1825 | CA | THR | B | 14 | 33.561 | 55.127 | 71.473 | 1.00 | 91.34 | 6 |
| ATOM | 1826 | CB | THR | B | 14 | 34.018 | 56.582 | 71.335 | 1.00 | 149.30 | 6 |
| ATOM | 1827 | OG1 | THR | B | 14 | 34.368 | 56.838 | 69.968 | 1.00 | 151.79 | 8 |
| ATOM | 1828 | CG2 | THR | B | 14 | 35.232 | 56.852 | 72.214 | 1.00 | 148.89 | 6 |
| ATOM | 1829 | C | THR | B | 14 | 33.351 | 54.802 | 72.946 | 1.00 | 93.15 | 6 |
| ATOM | 1830 | O | THR | B | 14 | 34.305 | 54.731 | 73.713 | 1.00 | 93.29 | 8 |
| ATOM | 1831 | N | THR | B | 15 | 32.095 | 54.612 | 73.332 | 1.00 | 108.52 | 7 |
| ATOM | 1832 | CA | THR | B | 15 | 31.741 | 54.251 | 74.701 | 1.00 | 109.63 | 6 |
| ATOM | 1833 | CB | THR | B | 15 | 31.501 | 52.723 | 74.807 | 1.00 | 158.82 | 6 |
| ATOM | 1834 | OG1 | THR | B | 15 | 32.586 | 52.022 | 74.187 | 1.00 | 160.26 | 8 |
| ATOM | 1835 | CG2 | THR | B | 15 | 30.196 | 52.331 | 74.119 | 1.00 | 158.82 | 6 |
| ATOM | 1836 | C | THR | B | 15 | 32.739 | 54.657 | 75.791 | 1.00 | 110.48 | 6 |
| ATOM | 1837 | O | THR | B | 15 | 33.354 | 53.804 | 76.432 | 1.00 | 111.01 | 8 |
| ATOM | 1838 | N | GLN | B | 16 | 32.893 | 55.959 | 76.008 | 1.00 | 86.50 | 7 |
| ATOM | 1839 | CA | GLN | B | 16 | 33.806 | 56.463 | 77.034 | 1.00 | 87.15 | 6 |
| ATOM | 1840 | CB | GLN | B | 16 | 33.877 | 57.996 | 76.949 | 1.00 | 90.84 | 6 |
| ATOM | 1841 | CG | GLN | B | 16 | 34.756 | 58.676 | 77.985 | 1.00 | 92.61 | 6 |
| ATOM | 1842 | CD | GLN | B | 16 | 36.183 | 58.158 | 77.984 | 1.00 | 94.30 | 6 |
| ATOM | 1843 | OE1 | GLN | B | 16 | 36.836 | 58.104 | 76.941 | 1.00 | 96.12 | 8 |
| ATOM | 1844 | NE2 | GLN | B | 16 | 36.679 | 57.783 | 79.159 | 1.00 | 95.03 | 7 |
| ATOM | 1845 | C | GLN | B | 16 | 33.307 | 56.022 | 78.414 | 1.00 | 88.26 | 6 |
| ATOM | 1846 | O | GLN | B | 16 | 33.879 | 56.382 | 79.442 | 1.00 | 88.50 | 8 |
| ATOM | 1847 | N | GLY | B | 17 | 32.237 | 55.232 | 78.426 | 1.00 | 144.12 | 7 |
| ATOM | 1848 | CA | GLY | B | 17 | 31.684 | 54.764 | 79.682 | 1.00 | 143.78 | 6 |
| ATOM | 1849 | C | GLY | B | 17 | 30.395 | 55.477 | 80.037 | 1.00 | 144.22 | 6 |
| ATOM | 1850 | O | GLY | B | 17 | 29.395 | 55.356 | 79.332 | 1.00 | 144.77 | 8 |
| ATOM | 1851 | N | ASP | B | 18 | 30.413 | 56.231 | 81.128 | 1.00 | 104.50 | 7 |
| ATOM | 1852 | CA | ASP | B | 18 | 29.223 | 56.951 | 81.561 | 1.00 | 103.73 | 6 |
| ATOM | 1853 | CB | ASP | B | 18 | 29.371 | 57.379 | 83.021 | 1.00 | 208.05 | 6 |
| ATOM | 1854 | CG | ASP | B | 18 | 30.681 | 58.084 | 83.288 | 1.00 | 208.87 | 6 |
| ATOM | 1855 | OD1 | ASP | B | 18 | 31.740 | 57.435 | 83.153 | 1.00 | 208.87 | 8 |
| ATOM | 1856 | OD2 | ASP | B | 18 | 30.651 | 59.284 | 83.632 | 1.00 | 208.87 | 8 |
| ATOM | 1857 | C | ASP | B | 18 | 28.871 | 58.164 | 80.702 | 1.00 | 102.50 | 6 |
| ATOM | 1858 | O | ASP | B | 18 | 27.779 | 58.714 | 80.836 | 1.00 | 102.98 | 8 |
| ATOM | 1859 | N | HIS | B | 19 | 29.792 | 58.590 | 79.836 | 1.00 | 46.05 | 7 |
| ATOM | 1860 | CA | HIS | B | 19 | 29.548 | 59.727 | 78.943 | 1.00 | 44.55 | 6 |
| ATOM | 1861 | CB | HIS | B | 19 | 29.669 | 61.056 | 79.703 | 1.00 | 142.23 | 6 |
| ATOM | 1862 | CG | HIS | B | 19 | 30.981 | 61.256 | 80.394 | 1.00 | 144.41 | 6 |
| ATOM | 1863 | CD2 | HIS | B | 19 | 32.128 | 60.536 | 80.370 | 1.00 | 145.87 | 6 |
| ATOM | 1864 | ND1 | HIS | B | 19 | 31.221 | 62.328 | 81.225 | 1.00 | 145.49 | 7 |
| ATOM | 1865 | CE1 | HIS | B | 19 | 32.457 | 62.262 | 81.684 | 1.00 | 146.38 | 6 |
| ATOM | 1866 | NE2 | HIS | B | 19 | 33.029 | 61.183 | 81.179 | 1.00 | 146.80 | 7 |
| ATOM | 1867 | C | HIS | B | 19 | 30.449 | 59.743 | 77.705 | 1.00 | 42.86 | 6 |
| ATOM | 1868 | O | HIS | B | 19 | 31.104 | 58.758 | 77.389 | 1.00 | 41.54 | 8 |
| ATOM | 1869 | N | TYR | B | 20 | 30.464 | 60.864 | 76.998 | 1.00 | 101.12 | 7 |
| ATOM | 1870 | CA | TYR | B | 20 | 31.275 | 61.004 | 75.793 | 1.00 | 98.97 | 6 |
| ATOM | 1871 | CB | TYR | B | 20 | 32.761 | 61.154 | 76.146 | 1.00 | 129.01 | 6 |
| ATOM | 1872 | CG | TYR | B | 20 | 33.590 | 61.769 | 75.031 | 1.00 | 130.15 | 6 |
| ATOM | 1873 | CD1 | TYR | B | 20 | 34.959 | 61.990 | 75.189 | 1.00 | 130.78 | 6 |
| ATOM | 1874 | CE1 | TYR | B | 20 | 35.718 | 62.591 | 74.171 | 1.00 | 131.57 | 6 |
| ATOM | 1875 | CD2 | TYR | B | 20 | 32.997 | 62.160 | 73.827 | 1.00 | 130.28 | 6 |
| ATOM | 1876 | CE2 | TYR | B | 20 | 33.740 | 62.759 | 72.808 | 1.00 | 130.52 | 6 |
| ATOM | 1877 | CZ | TYR | B | 20 | 35.099 | 62.974 | 72.981 | 1.00 | 130.95 | 6 |
| ATOM | 1878 | OH | TYR | B | 20 | 35.825 | 63.575 | 71.966 | 1.00 | 129.42 | 8 |
| ATOM | 1879 | C | TYR | B | 20 | 31.106 | 59.821 | 74.849 | 1.00 | 96.88 | 6 |
| ATOM | 1880 | O | TYR | B | 20 | 32.029 | 59.035 | 74.654 | 1.00 | 97.42 | 8 |
| ATOM | 1881 | N | GLY | B | 21 | 29.924 | 59.702 | 74.261 | 1.00 | 94.58 | 7 |
| ATOM | 1882 | CA | GLY | B | 21 | 29.669 | 58.615 | 73.339 | 1.00 | 91.23 | 6 |
| ATOM | 1883 | C | GLY | B | 21 | 30.045 | 58.982 | 71.918 | 1.00 | 90.19 | 6 |
| ATOM | 1884 | O | GLY | B | 21 | 29.182 | 59.139 | 71.058 | 1.00 | 90.24 | 8 |
| ATOM | 1885 | N | GLU | B | 22 | 31.342 | 59.124 | 71.676 | 1.00 | 82.21 | 7 |
| ATOM | 1886 | CA | GLU | B | 22 | 31.865 | 59.467 | 70.356 | 1.00 | 79.42 | 6 |
| ATOM | 1887 | CB | GLU | B | 22 | 33.386 | 59.547 | 70.456 | 1.00 | 80.54 | 6 |
| ATOM | 1888 | CG | GLU | B | 22 | 34.110 | 60.342 | 69.407 | 1.00 | 81.39 | 6 |
| ATOM | 1889 | CD | GLU | B | 22 | 35.596 | 60.408 | 69.716 | 1.00 | 82.92 | 6 |
| ATOM | 1890 | OE1 | GLU | B | 22 | 36.274 | 59.369 | 69.576 | 1.00 | 82.42 | 8 |
| ATOM | 1891 | OE2 | GLU | B | 22 | 36.086 | 61.489 | 70.118 | 1.00 | 84.93 | 8 |
| ATOM | 1892 | C | GLU | B | 22 | 31.438 | 58.371 | 69.368 | 1.00 | 77.68 | 6 |
| ATOM | 1893 | O | GLU | B | 22 | 31.832 | 57.211 | 69.502 | 1.00 | 77.30 | 8 |
| ATOM | 1894 | N | PHE | B | 23 | 30.617 | 58.736 | 68.387 | 1.00 | 48.34 | 7 |
| ATOM | 1895 | CA | PHE | B | 23 | 30.125 | 57.766 | 67.405 | 1.00 | 45.62 | 6 |
| ATOM | 1896 | CB | PHE | B | 23 | 28.607 | 57.648 | 67.541 | 1.00 | 54.52 | 6 |
| ATOM | 1897 | CG | PHE | B | 23 | 28.164 | 57.240 | 68.920 | 1.00 | 53.70 | 6 |
| ATOM | 1898 | CD1 | PHE | B | 23 | 26.861 | 57.435 | 69.330 | 1.00 | 54.29 | 6 |
| ATOM | 1899 | CD2 | PHE | B | 23 | 29.065 | 56.674 | 69.816 | 1.00 | 52.65 | 6 |
| ATOM | 1900 | CE1 | PHE | B | 23 | 26.458 | 57.077 | 70.611 | 1.00 | 55.28 | 6 |
| ATOM | 1901 | CE2 | PHE | B | 23 | 28.672 | 56.314 | 71.098 | 1.00 | 53.37 | 6 |
| ATOM | 1902 | CZ | PHE | B | 23 | 27.368 | 56.515 | 71.497 | 1.00 | 54.11 | 6 |
| ATOM | 1903 | C | PHE | B | 23 | 30.538 | 58.118 | 65.978 | 1.00 | 44.10 | 6 |
| ATOM | 1904 | O | PHE | B | 23 | 30.849 | 59.265 | 65.688 | 1.00 | 44.77 | 8 |
| ATOM | 1905 | N | VAL | B | 24 | 30.534 | 57.139 | 65.082 | 1.00 | 44.68 | 7 |
| ATOM | 1906 | CA | VAL | B | 24 | 30.993 | 57.388 | 63.726 | 1.00 | 43.27 | 6 |
| ATOM | 1907 | CB | VAL | B | 24 | 32.529 | 57.187 | 63.625 | 1.00 | 15.18 | 6 |
| ATOM | 1908 | CG1 | VAL | B | 24 | 32.988 | 57.381 | 62.216 | 1.00 | 13.87 | 6 |
| ATOM | 1909 | CG2 | VAL | B | 24 | 33.248 | 58.131 | 64.528 | 1.00 | 15.68 | 6 |
| ATOM | 1910 | C | VAL | B | 24 | 30.395 | 56.492 | 62.656 | 1.00 | 44.28 | 6 |
| ATOM | 1911 | O | VAL | B | 24 | 30.974 | 55.449 | 62.353 | 1.00 | 44.86 | 8 |
| ATOM | 1912 | N | LEU | B | 25 | 29.253 | 56.865 | 62.081 | 1.00 | 60.79 | 7 |
| ATOM | 1913 | CA | LEU | B | 25 | 28.698 | 56.055 | 60.994 | 1.00 | 60.09 | 6 |
| ATOM | 1914 | CB | LEU | B | 25 | 27.172 | 56.238 | 60.872 | 1.00 | 15.01 | 6 |
| ATOM | 1915 | CG | LEU | B | 25 | 26.568 | 57.265 | 59.920 | 1.00 | 13.87 | 6 |
| ATOM | 1916 | CD1 | LEU | B | 25 | 26.903 | 56.857 | 58.514 | 1.00 | 13.87 | 6 |
| ATOM | 1917 | CD2 | LEU | B | 25 | 25.053 | 57.330 | 60.085 | 1.00 | 13.87 | 6 |
| ATOM | 1918 | C | LEU | B | 25 | 29.476 | 56.695 | 59.848 | 1.00 | 61.13 | 6 |
| ATOM | 1919 | O | LEU | B | 25 | 29.722 | 57.903 | 59.889 | 1.00 | 60.97 | 8 |
| ATOM | 1920 | N | GLU | B | 26 | 29.866 | 55.924 | 58.832 | 1.00 | 89.90 | 7 |
| ATOM | 1921 | CA | GLU | B | 26 | 30.705 | 56.519 | 57.794 | 1.00 | 91.26 | 6 |
| ATOM | 1922 | CB | GLU | B | 26 | 32.037 | 55.774 | 57.778 | 1.00 | 102.03 | 6 |
| ATOM | 1923 | CG | GLU | B | 26 | 33.175 | 56.601 | 57.229 | 1.00 | 104.07 | 6 |
| ATOM | 1924 | CD | GLU | B | 26 | 34.422 | 55.782 | 57.030 | 1.00 | 107.14 | 6 |
| ATOM | 1925 | OE1 | GLU | B | 26 | 34.388 | 54.866 | 56.182 | 1.00 | 109.13 | 8 |
| ATOM | 1926 | OE2 | GLU | B | 26 | 35.428 | 56.044 | 57.724 | 1.00 | 107.71 | 8 |
| ATOM | 1927 | C | GLU | B | 26 | 30.263 | 56.838 | 56.348 | 1.00 | 90.05 | 6 |
| ATOM | 1928 | O | GLU | B | 26 | 29.431 | 57.728 | 56.157 | 1.00 | 90.37 | 8 |
| ATOM | 1929 | N | PRO | B | 27 | 30.785 | 56.125 | 55.317 | 1.00 | 56.95 | 7 |
| ATOM | 1930 | CD | PRO | B | 27 | 31.531 | 54.861 | 55.190 | 1.00 | 13.87 | 6 |
| ATOM | 1931 | CA | PRO | B | 27 | 30.309 | 56.550 | 53.993 | 1.00 | 58.52 | 6 |
| ATOM | 1932 | CB | PRO | B | 27 | 30.825 | 55.449 | 53.056 | 1.00 | 14.57 | 6 |
| ATOM | 1933 | CG | PRO | B | 27 | 32.052 | 54.963 | 53.766 | 1.00 | 13.87 | 6 |
| ATOM | 1934 | C | PRO | B | 27 | 28.822 | 56.836 | 53.851 | 1.00 | 58.78 | 6 |
| ATOM | 1935 | O | PRO | B | 27 | 27.965 | 55.972 | 54.047 | 1.00 | 59.53 | 8 |
| ATOM | 1936 | N | LEU | B | 28 | 28.536 | 58.086 | 53.519 | 1.00 | 37.49 | 7 |
| ATOM | 1937 | CA | LEU | B | 28 | 27.174 | 58.550 | 53.347 | 1.00 | 38.26 | 6 |
| ATOM | 1938 | CB | LEU | B | 28 | 26.741 | 59.309 | 54.611 | 1.00 | 21.14 | 6 |
| ATOM | 1939 | CG | LEU | B | 28 | 26.710 | 58.546 | 55.951 | 1.00 | 18.35 | 6 |
| ATOM | 1940 | CD1 | LEU | B | 28 | 26.465 | 59.481 | 57.103 | 1.00 | 18.15 | 6 |
| ATOM | 1941 | CD2 | LEU | B | 28 | 25.622 | 57.519 | 55.898 | 1.00 | 17.94 | 6 |
| ATOM | 1942 | C | LEU | B | 28 | 27.232 | 59.479 | 52.150 | 1.00 | 39.15 | 6 |
| ATOM | 1943 | O | LEU | B | 28 | 28.181 | 60.234 | 52.018 | 1.00 | 39.68 | 8 |
| ATOM | 1944 | N | GLU | B | 29 | 26.246 | 59.418 | 51.267 | 1.00 | 28.57 | 7 |
| ATOM | 1945 | CA | GLU | B | 29 | 26.255 | 60.294 | 50.095 | 1.00 | 30.85 | 6 |
| ATOM | 1946 | CB | GLU | B | 29 | 25.014 | 60.038 | 49.235 | 1.00 | 193.66 | 6 |
| ATOM | 1947 | CG | GLU | B | 29 | 25.037 | 60.724 | 47.871 | 1.00 | 198.20 | 6 |
| ATOM | 1948 | CD | GLU | B | 29 | 26.068 | 60.131 | 46.927 | 1.00 | 200.49 | 6 |
| ATOM | 1949 | OE1 | GLU | B | 29 | 27.268 | 60.137 | 47.276 | 1.00 | 202.11 | 8 |
| ATOM | 1950 | OE2 | GLU | B | 29 | 25.677 | 59.659 | 45.835 | 1.00 | 199.88 | 8 |
| ATOM | 1951 | C | GLU | B | 29 | 26.296 | 61.769 | 50.530 | 1.00 | 29.83 | 6 |
| ATOM | 1952 | O | GLU | B | 29 | 25.524 | 62.200 | 51.405 | 1.00 | 28.53 | 8 |
| ATOM | 1953 | N | ARG | B | 30 | 27.183 | 62.548 | 49.915 | 1.00 | 47.67 | 7 |
| ATOM | 1954 | CA | ARG | B | 30 | 27.313 | 63.950 | 50.295 | 1.00 | 48.20 | 6 |
| ATOM | 1955 | CB | ARG | B | 30 | 28.083 | 64.749 | 49.228 | 1.00 | 85.66 | 6 |
| ATOM | 1956 | CG | ARG | B | 30 | 27.338 | 64.986 | 47.926 | 1.00 | 89.54 | 6 |
| ATOM | 1957 | CD | ARG | B | 30 | 27.587 | 66.396 | 47.389 | 1.00 | 92.43 | 6 |
| ATOM | 1958 | NE | ARG | B | 30 | 26.693 | 66.724 | 46.275 | 1.00 | 95.87 | 7 |
| ATOM | 1959 | CZ | ARG | B | 30 | 26.707 | 67.874 | 45.602 | 1.00 | 96.56 | 6 |
| ATOM | 1960 | NH1 | ARG | B | 30 | 27.571 | 68.830 | 45.922 | 1.00 | 96.82 | 7 |
| ATOM | 1961 | NH2 | ARG | B | 30 | 25.863 | 68.065 | 44.597 | 1.00 | 96.67 | 7 |
| ATOM | 1962 | C | ARG | B | 30 | 25.958 | 64.596 | 50.553 | 1.00 | 45.28 | 6 |
| ATOM | 1963 | O | ARG | B | 30 | 25.218 | 64.905 | 49.629 | 1.00 | 44.63 | 8 |
| ATOM | 1964 | N | GLY | B | 31 | 25.619 | 64.788 | 51.816 | 1.00 | 66.33 | 7 |
| ATOM | 1965 | CA | GLY | B | 31 | 24.349 | 65.420 | 52.101 | 1.00 | 66.30 | 6 |
| ATOM | 1966 | C | GLY | B | 31 | 23.482 | 64.622 | 53.040 | 1.00 | 65.00 | 6 |
| ATOM | 1967 | O | GLY | B | 31 | 22.256 | 64.592 | 52.895 | 1.00 | 65.26 | 8 |
| ATOM | 1968 | N | PHE | B | 32 | 24.120 | 63.962 | 53.996 | 1.00 | 58.11 | 7 |
| ATOM | 1969 | CA | PHE | B | 32 | 23.403 | 63.171 | 54.977 | 1.00 | 55.81 | 6 |
| ATOM | 1970 | CB | PHE | B | 32 | 23.260 | 61.722 | 54.506 | 1.00 | 24.56 | 6 |
| ATOM | 1971 | CG | PHE | B | 32 | 22.180 | 61.538 | 53.483 | 1.00 | 21.18 | 6 |
| ATOM | 1972 | CD1 | PHE | B | 32 | 20.874 | 61.282 | 53.877 | 1.00 | 20.75 | 6 |
| ATOM | 1973 | CD2 | PHE | B | 32 | 22.451 | 61.701 | 52.126 | 1.00 | 19.17 | 6 |
| ATOM | 1974 | CE1 | PHE | B | 32 | 19.853 | 61.199 | 52.946 | 1.00 | 19.26 | 6 |
| ATOM | 1975 | CE2 | PHE | B | 32 | 21.431 | 61.623 | 51.180 | 1.00 | 17.56 | 6 |
| ATOM | 1976 | CZ | PHE | B | 32 | 20.131 | 61.371 | 51.592 | 1.00 | 18.01 | 6 |
| ATOM | 1977 | C | PHE | B | 32 | 24.156 | 63.259 | 56.277 | 1.00 | 55.18 | 6 |
| ATOM | 1978 | O | PHE | B | 32 | 23.590 | 63.048 | 57.347 | 1.00 | 56.33 | 8 |
| ATOM | 1979 | N | GLY | B | 33 | 25.439 | 63.580 | 56.189 | 1.00 | 33.58 | 7 |
| ATOM | 1980 | CA | GLY | B | 33 | 26.199 | 63.727 | 57.408 | 1.00 | 34.02 | 6 |
| ATOM | 1981 | C | GLY | B | 33 | 25.458 | 64.819 | 58.154 | 1.00 | 33.98 | 6 |
| ATOM | 1982 | O | GLY | B | 33 | 25.449 | 64.881 | 59.384 | 1.00 | 33.68 | 8 |
| ATOM | 1983 | N | VAL | B | 34 | 24.820 | 65.690 | 57.376 | 1.00 | 53.79 | 7 |
| ATOM | 1984 | CA | VAL | B | 34 | 24.043 | 66.790 | 57.919 | 1.00 | 53.45 | 6 |
| ATOM | 1985 | CB | VAL | B | 34 | 23.890 | 67.945 | 56.906 | 1.00 | 33.83 | 6 |
| ATOM | 1986 | CG1 | VAL | B | 34 | 23.298 | 69.148 | 57.586 | 1.00 | 35.07 | 6 |
| ATOM | 1987 | CG2 | VAL | B | 34 | 25.219 | 68.294 | 56.294 | 1.00 | 35.07 | 6 |
| ATOM | 1988 | C | VAL | B | 34 | 22.658 | 66.236 | 58.180 | 1.00 | 53.28 | 6 |
| ATOM | 1989 | O | VAL | B | 34 | 22.196 | 66.197 | 59.321 | 1.00 | 53.53 | 8 |
| ATOM | 1990 | N | THR | B | 35 | 22.023 | 65.781 | 57.100 | 1.00 | 54.49 | 7 |
| ATOM | 1991 | CA | THR | B | 35 | 20.665 | 65.249 | 57.137 | 1.00 | 53.19 | 6 |
| ATOM | 1992 | CB | THR | B | 35 | 20.323 | 64.431 | 55.896 | 1.00 | 47.42 | 6 |
| ATOM | 1993 | OG1 | THR | B | 35 | 20.965 | 64.990 | 54.751 | 1.00 | 47.14 | 8 |
| ATOM | 1994 | CG2 | THR | B | 35 | 18.832 | 64.472 | 55.663 | 1.00 | 47.59 | 6 |
| ATOM | 1995 | C | THR | B | 35 | 20.379 | 64.382 | 58.336 | 1.00 | 52.60 | 6 |
| ATOM | 1996 | O | THR | B | 35 | 19.224 | 64.223 | 58.725 | 1.00 | 53.16 | 8 |
| ATOM | 1997 | N | LEU | B | 36 | 21.412 | 63.806 | 58.926 | 1.00 | 20.23 | 7 |
| ATOM | 1998 | CA | LEU | B | 36 | 21.170 | 62.992 | 60.092 | 1.00 | 20.76 | 6 |
| ATOM | 1999 | CB | LEU | B | 36 | 21.763 | 61.602 | 59.919 | 1.00 | 46.64 | 6 |
| ATOM | 2000 | CG | LEU | B | 36 | 21.303 | 60.874 | 58.663 | 1.00 | 46.22 | 6 |
| ATOM | 2001 | CD1 | LEU | B | 36 | 21.674 | 59.399 | 58.803 | 1.00 | 46.91 | 6 |
| ATOM | 2002 | CD2 | LEU | B | 36 | 19.801 | 61.046 | 58.465 | 1.00 | 44.40 | 6 |
| ATOM | 2003 | C | LEU | B | 36 | 21.738 | 63.641 | 61.332 | 1.00 | 21.80 | 6 |
| ATOM | 2004 | O | LEU | B | 36 | 20.991 | 64.098 | 62.189 | 1.00 | 21.82 | 8 |
| ATOM | 2005 | N | GLY | B | 37 | 23.064 | 63.680 | 61.413 | 1.00 | 70.68 | 7 |
| ATOM | 2006 | CA | GLY | B | 37 | 23.743 | 64.256 | 62.558 | 1.00 | 71.90 | 6 |
| ATOM | 2007 | C | GLY | B | 37 | 22.994 | 65.360 | 63.274 | 1.00 | 72.46 | 6 |
| ATOM | 2008 | O | GLY | B | 37 | 22.953 | 65.375 | 64.503 | 1.00 | 72.33 | 8 |
| ATOM | 2009 | N | ASN | B | 38 | 22.386 | 66.277 | 62.524 | 1.00 | 65.39 | 7 |
| ATOM | 2010 | CA | ASN | B | 38 | 21.673 | 67.384 | 63.150 | 1.00 | 65.26 | 6 |
| ATOM | 2011 | CB | ASN | B | 38 | 21.300 | 68.446 | 62.113 | 1.00 | 25.63 | 6 |
| ATOM | 2012 | CG | ASN | B | 38 | 21.229 | 69.838 | 62.718 | 1.00 | 24.58 | 6 |
| ATOM | 2013 | OD1 | ASN | B | 38 | 20.472 | 70.697 | 62.258 | 1.00 | 26.11 | 8 |
| ATOM | 2014 | ND2 | ASN | B | 38 | 22.036 | 70.074 | 63.749 | 1.00 | 22.75 | 7 |
| ATOM | 2015 | C | ASN | B | 38 | 20.431 | 66.983 | 63.955 | 1.00 | 66.04 | 6 |
| ATOM | 2016 | O | ASN | B | 38 | 20.450 | 67.067 | 65.184 | 1.00 | 66.24 | 8 |
| ATOM | 2017 | N | PRO | B | 39 | 19.339 | 66.538 | 63.284 | 1.00 | 86.12 | 7 |
| ATOM | 2018 | CD | PRO | B | 39 | 19.158 | 66.243 | 61.850 | 1.00 | 27.75 | 6 |
| ATOM | 2019 | CA | PRO | B | 39 | 18.132 | 66.150 | 64.023 | 1.00 | 86.08 | 6 |
| ATOM | 2020 | CB | PRO | B | 39 | 17.337 | 65.378 | 62.985 | 1.00 | 27.67 | 6 |
| ATOM | 2021 | CG | PRO | B | 39 | 17.665 | 66.099 | 61.737 | 1.00 | 26.77 | 6 |
| ATOM | 2022 | C | PRO | B | 39 | 18.429 | 65.327 | 65.270 | 1.00 | 87.63 | 6 |
| ATOM | 2023 | O | PRO | B | 39 | 17.793 | 65.515 | 66.305 | 1.00 | 88.84 | 8 |
| ATOM | 2024 | N | LEU | B | 40 | 19.389 | 64.412 | 65.170 | 1.00 | 37.16 | 7 |
| ATOM | 2025 | CA | LEU | B | 40 | 19.771 | 63.610 | 66.327 | 1.00 | 38.01 | 6 |
| ATOM | 2026 | CB | LEU | B | 40 | 20.829 | 62.552 | 65.964 | 1.00 | 30.64 | 6 |
| ATOM | 2027 | CG | LEU | B | 40 | 20.398 | 61.361 | 65.106 | 1.00 | 30.84 | 6 |
| ATOM | 2028 | CD1 | LEU | B | 40 | 19.168 | 60.700 | 65.700 | 1.00 | 30.00 | 6 |
| ATOM | 2029 | CD2 | LEU | B | 40 | 20.101 | 61.836 | 63.705 | 1.00 | 31.53 | 6 |
| ATOM | 2030 | C | LEU | B | 40 | 20.341 | 64.569 | 67.361 | 1.00 | 38.69 | 6 |
| ATOM | 2031 | O | LEU | B | 40 | 19.853 | 64.629 | 68.481 | 1.00 | 38.27 | 8 |
| ATOM | 2032 | N | ARG | B | 41 | 21.369 | 65.321 | 66.971 | 1.00 | 55.38 | 7 |
| ATOM | 2033 | CA | ARG | B | 41 | 22.003 | 66.298 | 67.858 | 1.00 | 56.87 | 6 |
| ATOM | 2034 | CB | ARG | B | 41 | 23.079 | 67.091 | 67.089 | 1.00 | 81.26 | 6 |
| ATOM | 2035 | CG | ARG | B | 41 | 23.262 | 68.555 | 67.505 | 1.00 | 84.64 | 6 |
| ATOM | 2036 | CD | ARG | B | 41 | 22.236 | 69.446 | 66.809 | 1.00 | 87.81 | 6 |
| ATOM | 2037 | NE | ARG | B | 41 | 22.300 | 70.844 | 67.226 | 1.00 | 91.69 | 7 |
| ATOM | 2038 | CZ | ARG | B | 41 | 21.468 | 71.790 | 66.791 | 1.00 | 94.10 | 6 |
| ATOM | 2039 | NH1 | ARG | B | 41 | 20.507 | 71.488 | 65.927 | 1.00 | 95.61 | 7 |
| ATOM | 2040 | NH2 | ARG | B | 41 | 21.591 | 73.040 | 67.220 | 1.00 | 95.48 | 7 |
| ATOM | 2041 | C | ARG | B | 41 | 20.939 | 67.238 | 68.411 | 1.00 | 55.92 | 6 |
| ATOM | 2042 | O | ARG | B | 41 | 20.902 | 67.543 | 69.608 | 1.00 | 56.14 | 8 |
| ATOM | 2043 | N | ARG | B | 42 | 20.069 | 67.692 | 67.523 | 1.00 | 57.87 | 7 |
| ATOM | 2044 | CA | ARG | B | 42 | 19.005 | 68.579 | 67.920 | 1.00 | 57.13 | 6 |
| ATOM | 2045 | CB | ARG | B | 42 | 18.113 | 68.868 | 66.727 | 1.00 | 64.73 | 6 |
| ATOM | 2046 | CG | ARG | B | 42 | 17.240 | 70.072 | 66.892 | 1.00 | 65.29 | 6 |
| ATOM | 2047 | CD | ARG | B | 42 | 16.208 | 70.092 | 65.800 | 1.00 | 66.30 | 6 |
| ATOM | 2048 | NE | ARG | B | 42 | 15.518 | 71.364 | 65.771 | 1.00 | 68.31 | 7 |
| ATOM | 2049 | CZ | ARG | B | 42 | 16.147 | 72.517 | 65.607 | 1.00 | 70.27 | 6 |
| ATOM | 2050 | NH1 | ARG | B | 42 | 17.468 | 72.527 | 65.461 | 1.00 | 70.63 | 7 |
| ATOM | 2051 | NH2 | ARG | B | 42 | 15.462 | 73.651 | 65.588 | 1.00 | 71.14 | 7 |
| ATOM | 2052 | C | ARG | B | 42 | 18.229 | 67.833 | 68.994 | 1.00 | 56.62 | 6 |
| ATOM | 2053 | O | ARG | B | 42 | 18.336 | 68.155 | 70.175 | 1.00 | 57.25 | 8 |
| ATOM | 2054 | N | ILE | B | 43 | 17.476 | 66.817 | 68.576 | 1.00 | 77.76 | 7 |
| ATOM | 2055 | CA | ILE | B | 43 | 16.676 | 65.998 | 69.483 | 1.00 | 76.69 | 6 |
| ATOM | 2056 | CB | ILE | B | 43 | 16.165 | 64.741 | 68.776 | 1.00 | 64.03 | 6 |
| ATOM | 2057 | CG2 | ILE | B | 43 | 15.567 | 63.786 | 69.783 | 1.00 | 62.92 | 6 |
| ATOM | 2058 | CG1 | ILE | B | 43 | 15.147 | 65.138 | 67.707 | 1.00 | 65.81 | 6 |
| ATOM | 2059 | CD1 | ILE | B | 43 | 14.445 | 63.971 | 67.030 | 1.00 | 70.53 | 6 |
| ATOM | 2060 | C | ILE | B | 43 | 17.442 | 65.561 | 70.721 | 1.00 | 76.34 | 6 |
| ATOM | 2061 | O | ILE | B | 43 | 16.889 | 65.539 | 71.818 | 1.00 | 76.73 | 8 |
| ATOM | 2062 | N | LEU | B | 44 | 18.709 | 65.202 | 70.527 | 1.00 | 46.89 | 7 |
| ATOM | 2063 | CA | LEU | B | 44 | 19.601 | 64.771 | 71.604 | 1.00 | 46.25 | 6 |
| ATOM | 2064 | CB | LEU | B | 44 | 21.035 | 64.674 | 71.090 | 1.00 | 38.30 | 6 |
| ATOM | 2065 | CG | LEU | B | 44 | 21.711 | 63.320 | 70.966 | 1.00 | 39.26 | 6 |
| ATOM | 2066 | CD1 | LEU | B | 44 | 20.927 | 62.307 | 71.762 | 1.00 | 39.28 | 6 |
| ATOM | 2067 | CD2 | LEU | B | 44 | 21.826 | 62.931 | 69.505 | 1.00 | 39.06 | 6 |
| ATOM | 2068 | C | LEU | B | 44 | 19.593 | 65.742 | 72.778 | 1.00 | 46.18 | 6 |
| ATOM | 2069 | O | LEU | B | 44 | 19.448 | 65.327 | 73.932 | 1.00 | 45.77 | 8 |
| ATOM | 2070 | N | LEU | B | 45 | 19.768 | 67.030 | 72.467 | 1.00 | 43.02 | 7 |
| ATOM | 2071 | CA | LEU | B | 45 | 19.802 | 68.093 | 73.471 | 1.00 | 42.79 | 6 |
| ATOM | 2072 | CB | LEU | B | 45 | 20.227 | 69.412 | 72.835 | 1.00 | 59.53 | 6 |
| ATOM | 2073 | CG | LEU | B | 45 | 21.718 | 69.572 | 72.551 | 1.00 | 59.66 | 6 |
| ATOM | 2074 | CD1 | LEU | B | 45 | 21.947 | 70.993 | 72.028 | 1.00 | 59.91 | 6 |
| ATOM | 2075 | CD2 | LEU | B | 45 | 22.548 | 69.307 | 73.827 | 1.00 | 59.49 | 6 |
| ATOM | 2076 | C | LEU | B | 45 | 18.485 | 68.302 | 74.182 | 1.00 | 43.51 | 6 |
| ATOM | 2077 | O | LEU | B | 45 | 18.447 | 68.848 | 75.283 | 1.00 | 42.99 | 8 |
| ATOM | 2078 | N | SER | B | 46 | 17.410 | 67.867 | 73.539 | 1.00 | 139.02 | 7 |
| ATOM | 2079 | CA | SER | B | 46 | 16.076 | 68.000 | 74.095 | 1.00 | 140.04 | 6 |
| ATOM | 2080 | CB | SER | B | 46 | 15.203 | 68.840 | 73.160 | 1.00 | 52.11 | 6 |
| ATOM | 2081 | OG | SER | B | 46 | 15.746 | 70.140 | 73.005 | 1.00 | 52.48 | 8 |
| ATOM | 2082 | C | SER | B | 46 | 15.445 | 66.631 | 74.284 | 1.00 | 141.18 | 6 |
| ATOM | 2083 | O | SER | B | 46 | 16.113 | 65.664 | 74.662 | 1.00 | 141.89 | 8 |
| ATOM | 2084 | N | SER | B | 47 | 14.146 | 66.576 | 74.022 | 1.00 | 62.93 | 7 |
| ATOM | 2085 | CA | SER | B | 47 | 13.361 | 65.360 | 74.121 | 1.00 | 62.15 | 6 |
| ATOM | 2086 | CB | SER | B | 47 | 12.688 | 65.132 | 72.773 | 1.00 | 33.15 | 6 |
| ATOM | 2087 | OG | SER | B | 47 | 12.207 | 66.370 | 72.280 | 1.00 | 34.70 | 8 |
| ATOM | 2088 | C | SER | B | 47 | 14.147 | 64.116 | 74.558 | 1.00 | 61.77 | 6 |
| ATOM | 2089 | O | SER | B | 47 | 14.852 | 63.499 | 73.768 | 1.00 | 61.78 | 8 |
| ATOM | 2090 | N | ILE | B | 48 | 14.020 | 63.780 | 75.835 | 1.00 | 37.83 | 7 |
| ATOM | 2091 | CA | ILE | B | 48 | 14.660 | 62.615 | 76.439 | 1.00 | 37.39 | 6 |
| ATOM | 2092 | CB | ILE | B | 48 | 16.191 | 62.619 | 76.259 | 1.00 | 16.95 | 6 |
| ATOM | 2093 | CG2 | ILE | B | 48 | 16.860 | 61.854 | 77.420 | 1.00 | 17.05 | 6 |
| ATOM | 2094 | CG1 | ILE | B | 48 | 16.564 | 61.993 | 74.915 | 1.00 | 14.85 | 6 |
| ATOM | 2095 | CD1 | ILE | B | 48 | 18.001 | 61.528 | 74.848 | 1.00 | 15.56 | 6 |
| ATOM | 2096 | C | ILE | B | 48 | 14.346 | 62.631 | 77.930 | 1.00 | 39.08 | 6 |
| ATOM | 2097 | O | ILE | B | 48 | 14.724 | 63.554 | 78.638 | 1.00 | 39.18 | 8 |
| ATOM | 2098 | N | PRO | B | 49 | 13.663 | 61.595 | 78.430 | 1.00 | 39.29 | 7 |
| ATOM | 2099 | CD | PRO | B | 49 | 13.423 | 60.297 | 77.780 | 1.00 | 99.40 | 6 |
| ATOM | 2100 | CA | PRO | B | 49 | 13.312 | 61.537 | 79.855 | 1.00 | 40.69 | 6 |
| ATOM | 2101 | CB | PRO | B | 49 | 12.806 | 60.104 | 80.025 | 1.00 | 99.95 | 6 |
| ATOM | 2102 | CG | PRO | B | 49 | 13.533 | 59.355 | 78.940 | 1.00 | 100.22 | 6 |
| ATOM | 2103 | C | PRO | B | 49 | 14.463 | 61.872 | 80.803 | 1.00 | 41.75 | 6 |
| ATOM | 2104 | O | PRO | B | 49 | 15.623 | 61.592 | 80.490 | 1.00 | 41.14 | 8 |
| ATOM | 2105 | N | GLY | B | 50 | 14.132 | 62.464 | 81.956 | 1.00 | 70.15 | 7 |
| ATOM | 2106 | CA | GLY | B | 50 | 15.150 | 62.840 | 82.929 | 1.00 | 71.83 | 6 |
| ATOM | 2107 | C | GLY | B | 50 | 14.865 | 62.678 | 84.422 | 1.00 | 74.05 | 6 |
| ATOM | 2108 | O | GLY | B | 50 | 14.013 | 61.892 | 84.842 | 1.00 | 74.59 | 8 |
| ATOM | 2109 | N | THR | B | 51 | 15.615 | 63.435 | 85.222 | 1.00 | 119.01 | 7 |
| ATOM | 2110 | CA | THR | B | 51 | 15.527 | 63.441 | 86.688 | 1.00 | 118.26 | 6 |
| ATOM | 2111 | CB | THR | B | 51 | 16.147 | 62.177 | 87.314 | 1.00 | 46.81 | 6 |
| ATOM | 2112 | OG1 | THR | B | 51 | 16.614 | 62.481 | 88.640 | 1.00 | 46.78 | 8 |
| ATOM | 2113 | CG2 | THR | B | 51 | 17.319 | 61.690 | 86.468 | 1.00 | 46.66 | 6 |
| ATOM | 2114 | C | THR | B | 51 | 16.334 | 64.623 | 87.209 | 1.00 | 119.45 | 6 |
| ATOM | 2115 | O | THR | B | 51 | 17.516 | 64.475 | 87.534 | 1.00 | 119.94 | 8 |
| ATOM | 2116 | N | ALA | B | 52 | 15.706 | 65.790 | 87.303 | 1.00 | 103.75 | 7 |
| ATOM | 2117 | CA | ALA | B | 52 | 16.428 | 66.967 | 87.764 | 1.00 | 102.87 | 6 |
| ATOM | 2118 | CB | ALA | B | 52 | 16.697 | 67.892 | 86.582 | 1.00 | 152.39 | 6 |
| ATOM | 2119 | C | ALA | B | 52 | 15.774 | 67.756 | 88.889 | 1.00 | 102.49 | 6 |
| ATOM | 2120 | O | ALA | B | 52 | 14.593 | 68.104 | 88.815 | 1.00 | 102.50 | 8 |
| ATOM | 2121 | N | VAL | B | 53 | 16.556 | 68.038 | 89.930 | 1.00 | 62.89 | 7 |
| ATOM | 2122 | CA | VAL | B | 53 | 16.071 | 68.827 | 91.054 | 1.00 | 61.42 | 6 |
| ATOM | 2123 | CB | VAL | B | 53 | 17.251 | 69.332 | 91.935 | 1.00 | 38.24 | 6 |
| ATOM | 2124 | CG1 | VAL | B | 53 | 16.826 | 70.536 | 92.761 | 1.00 | 37.03 | 6 |
| ATOM | 2125 | CG2 | VAL | B | 53 | 17.734 | 68.211 | 92.852 | 1.00 | 37.95 | 6 |
| ATOM | 2126 | C | VAL | B | 53 | 15.378 | 70.011 | 90.404 | 1.00 | 62.14 | 6 |
| ATOM | 2127 | O | VAL | B | 53 | 15.778 | 70.444 | 89.323 | 1.00 | 61.57 | 8 |
| ATOM | 2128 | N | THR | B | 54 | 14.336 | 70.534 | 91.031 | 1.00 | 59.37 | 7 |
| ATOM | 2129 | CA | THR | B | 54 | 13.668 | 71.654 | 90.412 | 1.00 | 61.52 | 6 |
| ATOM | 2130 | CB | THR | B | 54 | 12.606 | 71.155 | 89.423 | 1.00 | 86.50 | 6 |
| ATOM | 2131 | OG1 | THR | B | 54 | 11.751 | 72.242 | 89.048 | 1.00 | 87.50 | 8 |
| ATOM | 2132 | CG2 | THR | B | 54 | 11.797 | 70.029 | 90.039 | 1.00 | 88.32 | 6 |
| ATOM | 2133 | C | THR | B | 54 | 13.053 | 72.667 | 91.362 | 1.00 | 62.88 | 6 |
| ATOM | 2134 | O | THR | B | 54 | 12.680 | 73.758 | 90.935 | 1.00 | 62.88 | 8 |
| ATOM | 2135 | N | SER | B | 55 | 12.968 | 72.324 | 92.645 | 1.00 | 68.07 | 7 |
| ATOM | 2136 | CA | SER | B | 55 | 12.390 | 73.228 | 93.641 | 1.00 | 68.95 | 6 |
| ATOM | 2137 | CB | SER | B | 55 | 10.867 | 73.069 | 93.645 | 1.00 | 66.80 | 6 |
| ATOM | 2138 | OG | SER | B | 55 | 10.243 | 74.033 | 94.471 | 1.00 | 68.93 | 8 |
| ATOM | 2139 | C | SER | B | 55 | 12.980 | 72.906 | 95.020 | 1.00 | 69.81 | 6 |
| ATOM | 2140 | O | SER | B | 55 | 12.898 | 71.767 | 95.477 | 1.00 | 70.50 | 8 |
| ATOM | 2141 | N | VAL | B | 56 | 13.567 | 73.900 | 95.688 | 1.00 | 39.05 | 7 |
| ATOM | 2142 | CA | VAL | B | 56 | 14.199 | 73.652 | 96.985 | 1.00 | 39.67 | 6 |
| ATOM | 2143 | CB | VAL | B | 56 | 15.710 | 74.006 | 96.953 | 1.00 | 37.61 | 6 |
| ATOM | 2144 | CG1 | VAL | B | 56 | 16.499 | 73.068 | 97.881 | 1.00 | 37.82 | 6 |
| ATOM | 2145 | CG2 | VAL | B | 56 | 16.228 | 73.935 | 95.537 | 1.00 | 36.61 | 6 |
| ATOM | 2146 | C | VAL | B | 56 | 13.607 | 74.376 | 98.185 | 1.00 | 42.16 | 6 |
| ATOM | 2147 | O | VAL | B | 56 | 12.975 | 75.413 | 98.062 | 1.00 | 41.71 | 8 |
| ATOM | 2148 | N | TYR | B | 57 | 13.835 | 73.801 | 99.360 | 1.00 | 90.24 | 7 |
| ATOM | 2149 | CA | TYR | B | 57 | 13.374 | 74.370 | 100.621 | 1.00 | 91.74 | 6 |
| ATOM | 2150 | CB | TYR | B | 57 | 12.043 | 73.739 | 101.064 | 1.00 | 94.82 | 6 |
| ATOM | 2151 | CG | TYR | B | 57 | 11.673 | 74.012 | 102.515 | 1.00 | 96.56 | 6 |
| ATOM | 2152 | CD1 | TYR | B | 57 | 10.822 | 73.157 | 103.210 | 1.00 | 97.93 | 6 |
| ATOM | 2153 | CE1 | TYR | B | 57 | 10.521 | 73.377 | 104.553 | 1.00 | 99.32 | 6 |
| ATOM | 2154 | CD2 | TYR | B | 57 | 12.204 | 75.101 | 103.204 | 1.00 | 96.87 | 6 |
| ATOM | 2155 | CE2 | TYR | B | 57 | 11.911 | 75.326 | 104.539 | 1.00 | 98.00 | 6 |
| ATOM | 2156 | CZ | TYR | B | 57 | 11.075 | 74.463 | 105.206 | 1.00 | 99.30 | 6 |
| ATOM | 2157 | OH | TYR | B | 57 | 10.812 | 74.681 | 106.533 | 1.00 | 99.95 | 8 |
| ATOM | 2158 | C | TYR | B | 57 | 14.439 | 74.108 | 101.682 | 1.00 | 93.31 | 6 |
| ATOM | 2159 | O | TYR | B | 57 | 14.880 | 72.976 | 101.870 | 1.00 | 93.22 | 8 |
| ATOM | 2160 | N | ILE | B | 58 | 14.853 | 75.167 | 102.364 | 1.00 | 150.74 | 7 |
| ATOM | 2161 | CA | ILE | B | 58 | 15.847 | 75.056 | 103.418 | 1.00 | 152.30 | 6 |
| ATOM | 2162 | CB | ILE | B | 58 | 17.147 | 75.777 | 103.039 | 1.00 | 89.36 | 6 |
| ATOM | 2163 | CG2 | ILE | B | 58 | 18.313 | 75.163 | 103.785 | 1.00 | 89.02 | 6 |
| ATOM | 2164 | CG1 | ILE | B | 58 | 17.395 | 75.661 | 101.538 | 1.00 | 90.55 | 6 |
| ATOM | 2165 | CD1 | ILE | B | 58 | 18.567 | 76.493 | 101.057 | 1.00 | 92.33 | 6 |
| ATOM | 2166 | C | ILE | B | 58 | 15.264 | 75.764 | 104.628 | 1.00 | 153.96 | 6 |
| ATOM | 2167 | O | ILE | B | 58 | 15.192 | 76.991 | 104.637 | 1.00 | 154.94 | 8 |
| ATOM | 2168 | N | GLU | B | 59 | 14.834 | 75.011 | 105.637 | 1.00 | 92.95 | 7 |
| ATOM | 2169 | CA | GLU | B | 59 | 14.267 | 75.642 | 106.820 | 1.00 | 93.68 | 6 |
| ATOM | 2170 | CB | GLU | B | 59 | 14.343 | 74.701 | 108.025 | 1.00 | 208.87 | 6 |
| ATOM | 2171 | CG | GLU | B | 59 | 14.156 | 75.393 | 109.381 | 1.00 | 208.87 | 6 |
| ATOM | 2172 | CD | GLU | B | 59 | 12.790 | 76.045 | 109.550 | 1.00 | 208.87 | 6 |
| ATOM | 2173 | OE1 | GLU | B | 59 | 12.400 | 76.865 | 108.690 | 1.00 | 208.87 | 8 |
| ATOM | 2174 | OE2 | GLU | B | 59 | 12.107 | 75.743 | 110.553 | 1.00 | 208.87 | 8 |
| ATOM | 2175 | C | GLU | B | 59 | 15.024 | 76.932 | 107.126 | 1.00 | 93.98 | 6 |
| ATOM | 2176 | O | GLU | B | 59 | 16.247 | 76.987 | 106.976 | 1.00 | 94.44 | 8 |
| ATOM | 2177 | N | ASP | B | 60 | 14.277 | 77.964 | 107.521 | 1.00 | 148.93 | 7 |
| ATOM | 2178 | CA | ASP | B | 60 | 14.792 | 79.291 | 107.882 | 1.00 | 148.06 | 6 |
| ATOM | 2179 | CB | ASP | B | 60 | 16.301 | 79.267 | 108.164 | 1.00 | 106.25 | 6 |
| ATOM | 2180 | CG | ASP | B | 60 | 16.636 | 78.550 | 109.453 | 1.00 | 106.75 | 6 |
| ATOM | 2181 | OD1 | ASP | B | 60 | 15.907 | 78.758 | 110.449 | 1.00 | 107.45 | 8 |
| ATOM | 2182 | OD2 | ASP | B | 60 | 17.631 | 77.793 | 109.476 | 1.00 | 105.57 | 8 |
| ATOM | 2183 | C | ASP | B | 60 | 14.491 | 80.426 | 106.915 | 1.00 | 148.12 | 6 |
| ATOM | 2184 | O | ASP | B | 60 | 13.353 | 80.887 | 106.839 | 1.00 | 148.37 | 8 |
| ATOM | 2185 | N | VAL | B | 61 | 15.503 | 80.873 | 106.175 | 1.00 | 35.04 | 7 |
| ATOM | 2186 | CA | VAL | B | 61 | 15.327 | 82.001 | 105.258 | 1.00 | 34.64 | 6 |
| ATOM | 2187 | CB | VAL | B | 61 | 16.673 | 82.667 | 104.912 | 1.00 | 65.03 | 6 |
| ATOM | 2188 | CG1 | VAL | B | 61 | 16.416 | 83.941 | 104.101 | 1.00 | 64.61 | 6 |
| ATOM | 2189 | CG2 | VAL | B | 61 | 17.452 | 82.984 | 106.194 | 1.00 | 65.20 | 6 |
| ATOM | 2190 | C | VAL | B | 61 | 14.586 | 81.755 | 103.955 | 1.00 | 35.20 | 6 |
| ATOM | 2191 | O | VAL | B | 61 | 14.997 | 80.944 | 103.119 | 1.00 | 34.12 | 8 |
| ATOM | 2192 | N | LEU | B | 62 | 13.501 | 82.513 | 103.807 | 1.00 | 82.75 | 7 |
| ATOM | 2193 | CA | LEU | B | 62 | 12.609 | 82.476 | 102.656 | 1.00 | 84.93 | 6 |
| ATOM | 2194 | CB | LEU | B | 62 | 11.786 | 83.768 | 102.600 | 1.00 | 180.46 | 6 |
| ATOM | 2195 | CG | LEU | B | 62 | 10.976 | 84.239 | 103.811 | 1.00 | 182.79 | 6 |
| ATOM | 2196 | CD1 | LEU | B | 62 | 10.711 | 85.733 | 103.688 | 1.00 | 183.84 | 6 |
| ATOM | 2197 | CD2 | LEU | B | 62 | 9.670 | 83.473 | 103.903 | 1.00 | 183.75 | 6 |
| ATOM | 2198 | C | LEU | B | 62 | 13.343 | 82.321 | 101.329 | 1.00 | 85.94 | 6 |
| ATOM | 2199 | O | LEU | B | 62 | 13.969 | 81.295 | 101.055 | 1.00 | 86.60 | 8 |
| ATOM | 2200 | N | HIS | B | 63 | 13.240 | 83.375 | 100.521 | 1.00 | 90.72 | 7 |
| ATOM | 2201 | CA | HIS | B | 63 | 13.831 | 83.481 | 99.189 | 1.00 | 91.10 | 6 |
| ATOM | 2202 | CB | HIS | B | 63 | 13.728 | 84.933 | 98.730 | 1.00 | 157.38 | 6 |
| ATOM | 2203 | CG | HIS | B | 63 | 12.415 | 85.571 | 99.067 | 1.00 | 158.97 | 6 |
| ATOM | 2204 | CD2 | HIS | B | 63 | 12.118 | 86.675 | 99.794 | 1.00 | 159.64 | 6 |
| ATOM | 2205 | ND1 | HIS | B | 63 | 11.207 | 85.053 | 98.650 | 1.00 | 160.02 | 7 |
| ATOM | 2206 | CE1 | HIS | B | 63 | 10.224 | 85.810 | 99.104 | 1.00 | 160.35 | 6 |
| ATOM | 2207 | NE2 | HIS | B | 63 | 10.750 | 86.801 | 99.802 | 1.00 | 160.12 | 7 |
| ATOM | 2208 | C | HIS | B | 63 | 15.270 | 82.984 | 99.043 | 1.00 | 91.43 | 6 |
| ATOM | 2209 | O | HIS | B | 63 | 15.876 | 82.494 | 99.995 | 1.00 | 91.89 | 8 |
| ATOM | 2210 | N | GLU | B | 64 | 15.811 | 83.128 | 97.837 | 1.00 | 84.24 | 7 |
| ATOM | 2211 | CA | GLU | B | 64 | 17.157 | 82.657 | 97.525 | 1.00 | 83.49 | 6 |
| ATOM | 2212 | CB | GLU | B | 64 | 17.241 | 82.319 | 96.036 | 1.00 | 208.87 | 6 |
| ATOM | 2213 | CG | GLU | B | 64 | 18.573 | 81.744 | 95.598 | 1.00 | 208.87 | 6 |
| ATOM | 2214 | CD | GLU | B | 64 | 18.638 | 81.523 | 94.101 | 1.00 | 208.87 | 6 |
| ATOM | 2215 | OE1 | GLU | B | 64 | 18.580 | 82.520 | 93.349 | 1.00 | 208.87 | 8 |
| ATOM | 2216 | OE2 | GLU | B | 64 | 18.743 | 80.353 | 93.675 | 1.00 | 208.87 | 8 |
| ATOM | 2217 | C | GLU | B | 64 | 18.304 | 83.597 | 97.885 | 1.00 | 82.97 | 6 |
| ATOM | 2218 | O | GLU | B | 64 | 19.358 | 83.150 | 98.336 | 1.00 | 83.53 | 8 |
| ATOM | 2219 | N | PHE | B | 65 | 18.105 | 84.891 | 97.665 | 1.00 | 95.11 | 7 |
| ATOM | 2220 | CA | PHE | B | 65 | 19.132 | 85.886 | 97.957 | 1.00 | 92.98 | 6 |
| ATOM | 2221 | CB | PHE | B | 65 | 18.695 | 87.244 | 97.399 | 1.00 | 165.17 | 6 |
| ATOM | 2222 | CG | PHE | B | 65 | 17.226 | 87.531 | 97.573 | 1.00 | 168.10 | 6 |
| ATOM | 2223 | CD1 | PHE | B | 65 | 16.702 | 87.847 | 98.824 | 1.00 | 168.98 | 6 |
| ATOM | 2224 | CD2 | PHE | B | 65 | 16.365 | 87.485 | 96.480 | 1.00 | 169.74 | 6 |
| ATOM | 2225 | CE1 | PHE | B | 65 | 15.343 | 88.112 | 98.982 | 1.00 | 170.31 | 6 |
| ATOM | 2226 | CE2 | PHE | B | 65 | 15.004 | 87.748 | 96.627 | 1.00 | 170.48 | 6 |
| ATOM | 2227 | CZ | PHE | B | 65 | 14.493 | 88.063 | 97.880 | 1.00 | 170.73 | 6 |
| ATOM | 2228 | C | PHE | B | 65 | 19.436 | 85.991 | 99.452 | 1.00 | 91.95 | 6 |
| ATOM | 2229 | O | PHE | B | 65 | 19.001 | 86.928 | 100.122 | 1.00 | 91.00 | 8 |
| ATOM | 2230 | N | SER | B | 66 | 20.197 | 85.027 | 99.967 | 1.00 | 48.43 | 7 |
| ATOM | 2231 | CA | SER | B | 66 | 20.549 | 85.003 | 101.381 | 1.00 | 46.27 | 6 |
| ATOM | 2232 | CB | SER | B | 66 | 19.443 | 84.347 | 102.204 | 1.00 | 57.99 | 6 |
| ATOM | 2233 | OG | SER | B | 66 | 19.889 | 84.077 | 103.529 | 1.00 | 58.99 | 8 |
| ATOM | 2234 | C | SER | B | 66 | 21.840 | 84.288 | 101.707 | 1.00 | 45.72 | 6 |
| ATOM | 2235 | O | SER | B | 66 | 22.649 | 83.984 | 100.838 | 1.00 | 45.98 | 8 |
| ATOM | 2236 | N | THR | B | 67 | 21.996 | 84.016 | 102.993 | 1.00 | 94.64 | 7 |
| ATOM | 2237 | CA | THR | B | 67 | 23.150 | 83.345 | 103.537 | 1.00 | 94.16 | 6 |
| ATOM | 2238 | CB | THR | B | 67 | 24.293 | 84.344 | 103.771 | 1.00 | 57.09 | 6 |
| ATOM | 2239 | OG1 | THR | B | 67 | 24.952 | 84.609 | 102.528 | 1.00 | 58.32 | 8 |
| ATOM | 2240 | CG2 | THR | B | 67 | 25.284 | 83.809 | 104.790 | 1.00 | 56.21 | 6 |
| ATOM | 2241 | C | THR | B | 67 | 22.701 | 82.780 | 104.870 | 1.00 | 95.18 | 6 |
| ATOM | 2242 | O | THR | B | 67 | 22.184 | 83.517 | 105.708 | 1.00 | 95.70 | 8 |
| ATOM | 2243 | N | ILE | B | 68 | 22.875 | 81.475 | 105.056 | 1.00 | 83.60 | 7 |
| ATOM | 2244 | CA | ILE | B | 68 | 22.499 | 80.829 | 106.308 | 1.00 | 82.48 | 6 |
| ATOM | 2245 | CB | ILE | B | 68 | 21.843 | 79.447 | 106.069 | 1.00 | 114.37 | 6 |
| ATOM | 2246 | CG2 | ILE | B | 68 | 21.663 | 78.724 | 107.390 | 1.00 | 114.07 | 6 |
| ATOM | 2247 | CG1 | ILE | B | 68 | 20.469 | 79.618 | 105.413 | 1.00 | 115.44 | 6 |
| ATOM | 2248 | CD1 | ILE | B | 68 | 20.500 | 80.251 | 104.039 | 1.00 | 117.44 | 6 |
| ATOM | 2249 | C | ILE | B | 68 | 23.764 | 80.651 | 107.135 | 1.00 | 82.86 | 6 |
| ATOM | 2250 | O | ILE | B | 68 | 24.690 | 79.955 | 106.716 | 1.00 | 82.59 | 8 |
| ATOM | 2251 | N | PRO | B | 69 | 23.824 | 81.301 | 108.312 | 1.00 | 80.58 | 7 |
| ATOM | 2252 | CD | PRO | B | 69 | 22.772 | 82.186 | 108.846 | 1.00 | 122.52 | 6 |
| ATOM | 2253 | CA | PRO | B | 69 | 24.964 | 81.245 | 109.234 | 1.00 | 79.17 | 6 |
| ATOM | 2254 | CB | PRO | B | 69 | 24.443 | 81.980 | 110.465 | 1.00 | 122.46 | 6 |
| ATOM | 2255 | CG | PRO | B | 69 | 23.526 | 82.995 | 109.875 | 1.00 | 122.69 | 6 |
| ATOM | 2256 | C | PRO | B | 69 | 25.420 | 79.822 | 109.557 | 1.00 | 79.52 | 6 |
| ATOM | 2257 | O | PRO | B | 69 | 24.619 | 78.884 | 109.563 | 1.00 | 79.86 | 8 |
| ATOM | 2258 | N | GLY | B | 70 | 26.713 | 79.670 | 109.830 | 1.00 | 87.91 | 7 |
| ATOM | 2259 | CA | GLY | B | 70 | 27.250 | 78.359 | 110.143 | 1.00 | 86.45 | 6 |
| ATOM | 2260 | C | GLY | B | 70 | 27.129 | 77.449 | 108.943 | 1.00 | 86.68 | 6 |
| ATOM | 2261 | O | GLY | B | 70 | 26.694 | 76.305 | 109.069 | 1.00 | 86.14 | 8 |
| ATOM | 2262 | N | VAL | B | 71 | 27.505 | 77.985 | 107.780 | 1.00 | 139.89 | 7 |
| ATOM | 2263 | CA | VAL | B | 71 | 27.471 | 77.287 | 106.491 | 1.00 | 139.35 | 6 |
| ATOM | 2264 | CB | VAL | B | 71 | 26.024 | 77.202 | 105.922 | 1.00 | 72.29 | 6 |
| ATOM | 2265 | CG1 | VAL | B | 71 | 26.024 | 76.399 | 104.645 | 1.00 | 73.35 | 6 |
| ATOM | 2266 | CG2 | VAL | B | 71 | 25.089 | 76.552 | 106.922 | 1.00 | 71.63 | 6 |
| ATOM | 2267 | C | VAL | B | 71 | 28.350 | 78.065 | 105.491 | 1.00 | 139.62 | 6 |
| ATOM | 2268 | O | VAL | B | 71 | 27.965 | 79.138 | 105.023 | 1.00 | 139.95 | 8 |
| ATOM | 2269 | N | LYS | B | 72 | 29.518 | 77.510 | 105.167 | 1.00 | 95.33 | 7 |
| ATOM | 2270 | CA | LYS | B | 72 | 30.485 | 78.134 | 104.259 | 1.00 | 94.09 | 6 |
| ATOM | 2271 | CB | LYS | B | 72 | 31.643 | 77.172 | 103.957 | 1.00 | 76.78 | 6 |
| ATOM | 2272 | CG | LYS | B | 72 | 32.731 | 77.814 | 103.081 | 1.00 | 77.29 | 6 |
| ATOM | 2273 | CD | LYS | B | 72 | 33.940 | 76.909 | 102.803 | 1.00 | 77.12 | 6 |
| ATOM | 2274 | CE | LYS | B | 72 | 34.947 | 77.591 | 101.853 | 1.00 | 75.39 | 6 |
| ATOM | 2275 | NZ | LYS | B | 72 | 36.160 | 76.765 | 101.616 | 1.00 | 73.12 | 7 |
| ATOM | 2276 | C | LYS | B | 72 | 29.966 | 78.685 | 102.934 | 1.00 | 93.33 | 6 |
| ATOM | 2277 | O | LYS | B | 72 | 30.579 | 79.586 | 102.358 | 1.00 | 93.92 | 8 |
| ATOM | 2278 | N | GLU | B | 73 | 28.858 | 78.151 | 102.437 | 1.00 | 48.05 | 7 |
| ATOM | 2279 | CA | GLU | B | 73 | 28.303 | 78.619 | 101.171 | 1.00 | 46.06 | 6 |
| ATOM | 2280 | CB | GLU | B | 73 | 27.736 | 77.444 | 100.379 | 1.00 | 61.85 | 6 |
| ATOM | 2281 | CG | GLU | B | 73 | 28.775 | 76.626 | 99.653 | 1.00 | 62.12 | 6 |
| ATOM | 2282 | CD | GLU | B | 73 | 29.762 | 75.946 | 100.576 | 1.00 | 61.73 | 6 |
| ATOM | 2283 | OE1 | GLU | B | 73 | 30.707 | 75.331 | 100.051 | 1.00 | 60.25 | 8 |
| ATOM | 2284 | OE2 | GLU | B | 73 | 29.602 | 76.016 | 101.812 | 1.00 | 62.43 | 8 |
| ATOM | 2285 | C | GLU | B | 73 | 27.215 | 79.662 | 101.360 | 1.00 | 45.75 | 6 |
| ATOM | 2286 | O | GLU | B | 73 | 27.114 | 80.294 | 102.413 | 1.00 | 46.03 | 8 |
| ATOM | 2287 | N | ASP | B | 74 | 26.413 | 79.843 | 100.317 | 1.00 | 48.39 | 7 |
| ATOM | 2288 | CA | ASP | B | 74 | 25.303 | 80.783 | 100.336 | 1.00 | 48.20 | 6 |
| ATOM | 2289 | CB | ASP | B | 74 | 25.754 | 82.199 | 99.943 | 1.00 | 100.71 | 6 |
| ATOM | 2290 | CG | ASP | B | 74 | 26.104 | 82.330 | 98.459 | 1.00 | 103.97 | 6 |
| ATOM | 2291 | OD1 | ASP | B | 74 | 27.091 | 81.708 | 98.015 | 1.00 | 106.12 | 8 |
| ATOM | 2292 | OD2 | ASP | B | 74 | 25.393 | 83.062 | 97.734 | 1.00 | 104.82 | 8 |
| ATOM | 2293 | C | ASP | B | 74 | 24.294 | 80.277 | 99.333 | 1.00 | 47.37 | 6 |
| ATOM | 2294 | O | ASP | B | 74 | 24.651 | 79.984 | 98.199 | 1.00 | 47.73 | 8 |
| ATOM | 2295 | N | VAL | B | 75 | 23.042 | 80.169 | 99.760 | 1.00 | 64.39 | 7 |
| ATOM | 2296 | CA | VAL | B | 75 | 21.953 | 79.695 | 98.909 | 1.00 | 63.43 | 6 |
| ATOM | 2297 | CB | VAL | B | 75 | 20.794 | 80.729 | 98.850 | 1.00 | 47.93 | 6 |
| ATOM | 2298 | CG1 | VAL | B | 75 | 19.467 | 80.017 | 98.584 | 1.00 | 47.57 | 6 |
| ATOM | 2299 | CG2 | VAL | B | 75 | 20.724 | 81.509 | 100.145 | 1.00 | 48.16 | 6 |
| ATOM | 2300 | C | VAL | B | 75 | 22.367 | 79.352 | 97.472 | 1.00 | 63.96 | 6 |
| ATOM | 2301 | O | VAL | B | 75 | 22.440 | 78.179 | 97.115 | 1.00 | 64.80 | 8 |
| ATOM | 2302 | N | VAL | B | 76 | 22.650 | 80.362 | 96.653 | 1.00 | 56.51 | 7 |
| ATOM | 2303 | CA | VAL | B | 76 | 23.028 | 80.120 | 95.257 | 1.00 | 56.80 | 6 |
| ATOM | 2304 | CB | VAL | B | 76 | 23.573 | 81.398 | 94.585 | 1.00 | 61.33 | 6 |
| ATOM | 2305 | CG1 | VAL | B | 76 | 23.925 | 81.118 | 93.128 | 1.00 | 62.03 | 6 |
| ATOM | 2306 | CG2 | VAL | B | 76 | 22.537 | 82.490 | 94.658 | 1.00 | 60.79 | 6 |
| ATOM | 2307 | C | VAL | B | 76 | 24.036 | 78.993 | 95.028 | 1.00 | 57.25 | 6 |
| ATOM | 2308 | O | VAL | B | 76 | 23.783 | 78.110 | 94.212 | 1.00 | 58.30 | 8 |
| ATOM | 2309 | N | GLU | B | 77 | 25.171 | 79.027 | 95.729 | 1.00 | 35.97 | 7 |
| ATOM | 2310 | CA | GLU | B | 77 | 26.194 | 77.995 | 95.582 | 1.00 | 35.90 | 6 |
| ATOM | 2311 | CB | GLU | B | 77 | 27.321 | 78.183 | 96.582 | 1.00 | 69.87 | 6 |
| ATOM | 2312 | CG | GLU | B | 77 | 28.508 | 77.299 | 96.281 | 1.00 | 73.10 | 6 |
| ATOM | 2313 | CD | GLU | B | 77 | 29.192 | 77.696 | 94.990 | 1.00 | 76.53 | 6 |
| ATOM | 2314 | OE1 | GLU | B | 77 | 28.482 | 77.915 | 93.986 | 1.00 | 78.26 | 8 |
| ATOM | 2315 | OE2 | GLU | B | 77 | 30.439 | 77.788 | 94.972 | 1.00 | 78.38 | 8 |
| ATOM | 2316 | C | GLU | B | 77 | 25.564 | 76.640 | 95.805 | 1.00 | 36.73 | 6 |
| ATOM | 2317 | O | GLU | B | 77 | 25.716 | 75.749 | 94.982 | 1.00 | 38.73 | 8 |
| ATOM | 2318 | N | ILE | B | 78 | 24.863 | 76.479 | 96.926 | 1.00 | 55.17 | 7 |
| ATOM | 2319 | CA | ILE | B | 78 | 24.170 | 75.224 | 97.211 | 1.00 | 53.98 | 6 |
| ATOM | 2320 | CB | ILE | B | 78 | 23.296 | 75.295 | 98.500 | 1.00 | 56.27 | 6 |
| ATOM | 2321 | CG2 | ILE | B | 78 | 22.354 | 74.123 | 98.541 | 1.00 | 57.67 | 6 |
| ATOM | 2322 | CG1 | ILE | B | 78 | 24.160 | 75.269 | 99.767 | 1.00 | 56.37 | 6 |
| ATOM | 2323 | CD1 | ILE | B | 78 | 24.404 | 76.621 | 100.397 | 1.00 | 56.85 | 6 |
| ATOM | 2324 | C | ILE | B | 78 | 23.237 | 75.032 | 96.024 | 1.00 | 53.97 | 6 |
| ATOM | 2325 | O | ILE | B | 78 | 23.360 | 74.061 | 95.281 | 1.00 | 54.16 | 8 |
| ATOM | 2326 | N | ILE | B | 79 | 22.319 | 75.987 | 95.857 | 1.00 | 65.75 | 7 |
| ATOM | 2327 | CA | ILE | B | 79 | 21.336 | 75.997 | 94.768 | 1.00 | 65.99 | 6 |
| ATOM | 2328 | CB | ILE | B | 79 | 20.848 | 77.444 | 94.451 | 1.00 | 69.77 | 6 |
| ATOM | 2329 | CG2 | ILE | B | 79 | 20.368 | 77.526 | 93.008 | 1.00 | 69.39 | 6 |
| ATOM | 2330 | CG1 | ILE | B | 79 | 19.730 | 77.872 | 95.415 | 1.00 | 70.43 | 6 |
| ATOM | 2331 | CD1 | ILE | B | 79 | 18.324 | 77.478 | 94.988 | 1.00 | 73.67 | 6 |
| ATOM | 2332 | C | ILE | B | 79 | 21.931 | 75.424 | 93.496 | 1.00 | 67.00 | 6 |
| ATOM | 2333 | O | ILE | B | 79 | 21.353 | 74.534 | 92.878 | 1.00 | 67.69 | 8 |
| ATOM | 2334 | N | LEU | B | 80 | 23.086 | 75.956 | 93.108 | 1.00 | 70.57 | 7 |
| ATOM | 2335 | CA | LEU | B | 80 | 23.781 | 75.513 | 91.907 | 1.00 | 69.02 | 6 |
| ATOM | 2336 | CB | LEU | B | 80 | 24.942 | 76.467 | 91.597 | 1.00 | 26.59 | 6 |
| ATOM | 2337 | CG | LEU | B | 80 | 24.585 | 77.959 | 91.502 | 1.00 | 24.04 | 6 |
| ATOM | 2338 | CD1 | LEU | B | 80 | 25.744 | 78.700 | 90.914 | 1.00 | 25.12 | 6 |
| ATOM | 2339 | CD2 | LEU | B | 80 | 23.360 | 78.177 | 90.643 | 1.00 | 21.84 | 6 |
| ATOM | 2340 | C | LEU | B | 80 | 24.270 | 74.073 | 92.079 | 1.00 | 69.55 | 6 |
| ATOM | 2341 | O | LEU | B | 80 | 24.059 | 73.241 | 91.203 | 1.00 | 70.67 | 8 |
| ATOM | 2342 | N | ASN | B | 81 | 24.917 | 73.777 | 93.202 | 1.00 | 32.83 | 7 |
| ATOM | 2343 | CA | ASN | B | 81 | 25.371 | 72.416 | 93.465 | 1.00 | 32.74 | 6 |
| ATOM | 2344 | CB | ASN | B | 81 | 25.758 | 72.241 | 94.927 | 1.00 | 53.32 | 6 |
| ATOM | 2345 | CG | ASN | B | 81 | 27.021 | 72.955 | 95.280 | 1.00 | 55.49 | 6 |
| ATOM | 2346 | OD1 | ASN | B | 81 | 28.101 | 72.609 | 94.805 | 1.00 | 55.56 | 8 |
| ATOM | 2347 | ND2 | ASN | B | 81 | 26.902 | 73.964 | 96.123 | 1.00 | 57.98 | 7 |
| ATOM | 2348 | C | ASN | B | 81 | 24.210 | 71.474 | 93.185 | 1.00 | 32.80 | 6 |
| ATOM | 2349 | O | ASN | B | 81 | 24.279 | 70.598 | 92.322 | 1.00 | 32.36 | 8 |
| ATOM | 2350 | N | LEU | B | 82 | 23.142 | 71.656 | 93.943 | 1.00 | 63.41 | 7 |
| ATOM | 2351 | CA | LEU | B | 82 | 21.959 | 70.843 | 93.784 | 1.00 | 63.31 | 6 |
| ATOM | 2352 | CB | LEU | B | 82 | 20.752 | 71.580 | 94.373 | 1.00 | 53.67 | 6 |
| ATOM | 2353 | CG | LEU | B | 82 | 19.575 | 70.783 | 94.940 | 1.00 | 53.30 | 6 |
| ATOM | 2354 | CD1 | LEU | B | 82 | 20.075 | 69.750 | 95.942 | 1.00 | 54.27 | 6 |
| ATOM | 2355 | CD2 | LEU | B | 82 | 18.611 | 71.744 | 95.612 | 1.00 | 52.12 | 6 |
| ATOM | 2356 | C | LEU | B | 82 | 21.759 | 70.585 | 92.293 | 1.00 | 63.65 | 6 |
| ATOM | 2357 | O | LEU | B | 82 | 21.578 | 69.447 | 91.883 | 1.00 | 63.99 | 8 |
| ATOM | 2358 | N | LYS | B | 83 | 21.827 | 71.642 | 91.484 | 1.00 | 50.46 | 7 |
| ATOM | 2359 | CA | LYS | B | 83 | 21.639 | 71.525 | 90.036 | 1.00 | 50.43 | 6 |
| ATOM | 2360 | CB | LYS | B | 83 | 21.912 | 72.862 | 89.345 | 1.00 | 61.05 | 6 |
| ATOM | 2361 | CG | LYS | B | 83 | 20.787 | 73.880 | 89.423 | 1.00 | 61.79 | 6 |
| ATOM | 2362 | CD | LYS | B | 83 | 21.137 | 75.141 | 88.614 | 1.00 | 63.49 | 6 |
| ATOM | 2363 | CE | LYS | B | 83 | 21.394 | 74.820 | 87.123 | 1.00 | 65.14 | 6 |
| ATOM | 2364 | NZ | LYS | B | 83 | 21.635 | 76.001 | 86.224 | 1.00 | 64.59 | 7 |
| ATOM | 2365 | C | LYS | B | 83 | 22.508 | 70.445 | 89.394 | 1.00 | 50.69 | 6 |
| ATOM | 2366 | O | LYS | B | 83 | 22.093 | 69.813 | 88.426 | 1.00 | 50.40 | 8 |
| ATOM | 2367 | N | GLU | B | 84 | 23.710 | 70.238 | 89.922 | 1.00 | 72.59 | 7 |
| ATOM | 2368 | CA | GLU | B | 84 | 24.602 | 69.220 | 89.384 | 1.00 | 73.33 | 6 |
| ATOM | 2369 | CB | GLU | B | 84 | 26.060 | 69.599 | 89.630 | 1.00 | 68.79 | 6 |
| ATOM | 2370 | CG | GLU | B | 84 | 26.556 | 70.775 | 88.821 | 1.00 | 68.46 | 6 |
| ATOM | 2371 | CD | GLU | B | 84 | 28.062 | 70.971 | 88.946 | 1.00 | 67.59 | 6 |
| ATOM | 2372 | OE1 | GLU | B | 84 | 28.553 | 71.199 | 90.078 | 1.00 | 66.17 | 8 |
| ATOM | 2373 | OE2 | GLU | B | 84 | 28.754 | 70.894 | 87.904 | 1.00 | 66.71 | 8 |
| ATOM | 2374 | C | GLU | B | 84 | 24.336 | 67.853 | 90.009 | 1.00 | 74.24 | 6 |
| ATOM | 2375 | O | GLU | B | 84 | 25.118 | 66.920 | 89.839 | 1.00 | 75.23 | 8 |
| ATOM | 2376 | N | LEU | B | 85 | 23.236 | 67.744 | 90.744 | 1.00 | 56.01 | 7 |
| ATOM | 2377 | CA | LEU | B | 85 | 22.853 | 66.495 | 91.398 | 1.00 | 55.89 | 6 |
| ATOM | 2378 | CB | LEU | B | 85 | 21.736 | 66.768 | 92.400 | 1.00 | 82.41 | 6 |
| ATOM | 2379 | CG | LEU | B | 85 | 21.185 | 65.612 | 93.226 | 1.00 | 83.62 | 6 |
| ATOM | 2380 | CD1 | LEU | B | 85 | 22.252 | 65.113 | 94.193 | 1.00 | 84.58 | 6 |
| ATOM | 2381 | CD2 | LEU | B | 85 | 19.956 | 66.085 | 93.988 | 1.00 | 83.69 | 6 |
| ATOM | 2382 | C | LEU | B | 85 | 22.353 | 65.519 | 90.346 | 1.00 | 56.47 | 6 |
| ATOM | 2383 | O | LEU | B | 85 | 21.671 | 65.932 | 89.412 | 1.00 | 56.46 | 8 |
| ATOM | 2384 | N | VAL | B | 86 | 22.686 | 64.235 | 90.482 | 1.00 | 58.30 | 7 |
| ATOM | 2385 | CA | VAL | B | 86 | 22.227 | 63.229 | 89.516 | 1.00 | 58.92 | 6 |
| ATOM | 2386 | CB | VAL | B | 86 | 23.416 | 62.561 | 88.752 | 1.00 | 28.71 | 6 |
| ATOM | 2387 | CG1 | VAL | B | 86 | 22.940 | 62.023 | 87.403 | 1.00 | 28.70 | 6 |
| ATOM | 2388 | CG2 | VAL | B | 86 | 24.543 | 63.547 | 88.545 | 1.00 | 26.79 | 6 |
| ATOM | 2389 | C | VAL | B | 86 | 21.422 | 62.141 | 90.237 | 1.00 | 60.35 | 6 |
| ATOM | 2390 | O | VAL | B | 86 | 21.984 | 61.253 | 90.878 | 1.00 | 59.96 | 8 |
| ATOM | 2391 | N | VAL | B | 87 | 20.101 | 62.229 | 90.135 | 1.00 | 51.71 | 7 |
| ATOM | 2392 | CA | VAL | B | 87 | 19.212 | 61.267 | 90.775 | 1.00 | 53.76 | 6 |
| ATOM | 2393 | CB | VAL | B | 87 | 17.954 | 61.954 | 91.369 | 1.00 | 59.83 | 6 |
| ATOM | 2394 | CG1 | VAL | B | 87 | 17.010 | 60.920 | 91.937 | 1.00 | 60.81 | 6 |
| ATOM | 2395 | CG2 | VAL | B | 87 | 18.352 | 62.914 | 92.461 | 1.00 | 60.06 | 6 |
| ATOM | 2396 | C | VAL | B | 87 | 18.753 | 60.286 | 89.722 | 1.00 | 56.80 | 6 |
| ATOM | 2397 | O | VAL | B | 87 | 18.842 | 60.573 | 88.532 | 1.00 | 56.99 | 8 |
| ATOM | 2398 | N | ARG | B | 88 | 18.263 | 59.133 | 90.167 | 1.00 | 109.23 | 7 |
| ATOM | 2399 | CA | ARG | B | 88 | 17.756 | 58.084 | 89.282 | 1.00 | 111.52 | 6 |
| ATOM | 2400 | CB | ARG | B | 88 | 18.820 | 57.022 | 89.069 | 1.00 | 100.13 | 6 |
| ATOM | 2401 | CG | ARG | B | 88 | 18.348 | 55.818 | 88.316 | 1.00 | 101.90 | 6 |
| ATOM | 2402 | CD | ARG | B | 88 | 19.463 | 54.818 | 88.303 | 1.00 | 103.39 | 6 |
| ATOM | 2403 | NE | ARG | B | 88 | 19.096 | 53.583 | 87.638 | 1.00 | 105.95 | 7 |
| ATOM | 2404 | CZ | ARG | B | 88 | 19.944 | 52.583 | 87.443 | 1.00 | 107.64 | 6 |
| ATOM | 2405 | NH1 | ARG | B | 88 | 21.198 | 52.691 | 87.869 | 1.00 | 108.21 | 7 |
| ATOM | 2406 | NH2 | ARG | B | 88 | 19.543 | 51.483 | 86.820 | 1.00 | 108.48 | 7 |
| ATOM | 2407 | C | ARG | B | 88 | 16.541 | 57.465 | 89.958 | 1.00 | 113.37 | 6 |
| ATOM | 2408 | O | ARG | B | 88 | 16.484 | 57.404 | 91.186 | 1.00 | 113.54 | 8 |
| ATOM | 2409 | N | PHE | B | 89 | 15.578 | 56.985 | 89.177 | 1.00 | 115.12 | 7 |
| ATOM | 2410 | CA | PHE | B | 89 | 14.381 | 56.437 | 89.796 | 1.00 | 116.99 | 6 |
| ATOM | 2411 | CB | PHE | B | 89 | 13.196 | 57.380 | 89.500 | 1.00 | 73.14 | 6 |
| ATOM | 2412 | CG | PHE | B | 89 | 12.686 | 57.336 | 88.074 | 1.00 | 73.17 | 6 |
| ATOM | 2413 | CD1 | PHE | B | 89 | 11.592 | 58.120 | 87.704 | 1.00 | 73.82 | 6 |
| ATOM | 2414 | CD2 | PHE | B | 89 | 13.253 | 56.495 | 87.120 | 1.00 | 73.06 | 6 |
| ATOM | 2415 | CE1 | PHE | B | 89 | 11.064 | 58.069 | 86.418 | 1.00 | 73.82 | 6 |
| ATOM | 2416 | CE2 | PHE | B | 89 | 12.735 | 56.432 | 85.827 | 1.00 | 73.37 | 6 |
| ATOM | 2417 | CZ | PHE | B | 89 | 11.635 | 57.222 | 85.476 | 1.00 | 73.73 | 6 |
| ATOM | 2418 | C | PHE | B | 89 | 14.001 | 54.976 | 89.506 | 1.00 | 119.15 | 6 |
| ATOM | 2419 | O | PHE | B | 89 | 12.822 | 54.619 | 89.549 | 1.00 | 119.44 | 8 |
| ATOM | 2420 | N | LEU | B | 90 | 14.995 | 54.128 | 89.249 | 1.00 | 130.10 | 7 |
| ATOM | 2421 | CA | LEU | B | 90 | 14.734 | 52.720 | 88.949 | 1.00 | 131.31 | 6 |
| ATOM | 2422 | CB | LEU | B | 90 | 13.990 | 52.058 | 90.108 | 1.00 | 161.35 | 6 |
| ATOM | 2423 | CG | LEU | B | 90 | 14.742 | 52.079 | 91.438 | 1.00 | 162.04 | 6 |
| ATOM | 2424 | CD1 | LEU | B | 90 | 13.912 | 51.378 | 92.491 | 1.00 | 162.42 | 6 |
| ATOM | 2425 | CD2 | LEU | B | 90 | 16.090 | 51.392 | 91.286 | 1.00 | 162.37 | 6 |
| ATOM | 2426 | C | LEU | B | 90 | 13.906 | 52.648 | 87.671 | 1.00 | 132.86 | 6 |
| ATOM | 2427 | O | LEU | B | 90 | 14.464 | 52.494 | 86.584 | 1.00 | 132.98 | 8 |
| ATOM | 2428 | N | ASP | B | 91 | 12.582 | 52.752 | 87.803 | 1.00 | 128.55 | 7 |
| ATOM | 2429 | CA | ASP | B | 91 | 11.689 | 52.763 | 86.638 | 1.00 | 128.88 | 6 |
| ATOM | 2430 | CB | ASP | B | 91 | 12.144 | 51.738 | 85.594 | 1.00 | 154.78 | 6 |
| ATOM | 2431 | CG | ASP | B | 91 | 11.860 | 52.197 | 84.172 | 1.00 | 155.70 | 6 |
| ATOM | 2432 | OD1 | ASP | B | 91 | 10.676 | 52.416 | 83.835 | 1.00 | 155.84 | 8 |
| ATOM | 2433 | OD2 | ASP | B | 91 | 12.824 | 52.355 | 83.390 | 1.00 | 156.27 | 8 |
| ATOM | 2434 | C | ASP | B | 91 | 10.191 | 52.576 | 86.878 | 1.00 | 129.05 | 6 |
| ATOM | 2435 | O | ASP | B | 91 | 9.505 | 51.976 | 86.055 | 1.00 | 129.10 | 8 |
| ATOM | 2436 | N | PRO | B | 92 | 9.659 | 53.079 | 88.004 | 1.00 | 52.86 | 7 |
| ATOM | 2437 | CD | PRO | B | 92 | 10.297 | 53.428 | 89.286 | 1.00 | 163.39 | 6 |
| ATOM | 2438 | CA | PRO | B | 92 | 8.217 | 52.880 | 88.180 | 1.00 | 52.64 | 6 |
| ATOM | 2439 | CB | PRO | B | 92 | 8.073 | 52.807 | 89.694 | 1.00 | 163.65 | 6 |
| ATOM | 2440 | CG | PRO | B | 92 | 9.110 | 53.772 | 90.155 | 1.00 | 163.70 | 6 |
| ATOM | 2441 | C | PRO | B | 92 | 7.411 | 54.028 | 87.566 | 1.00 | 52.65 | 6 |
| ATOM | 2442 | O | PRO | B | 92 | 7.342 | 54.172 | 86.345 | 1.00 | 52.29 | 8 |
| ATOM | 2443 | N | ARG | B | 93 | 6.794 | 54.824 | 88.433 | 1.00 | 159.93 | 7 |
| ATOM | 2444 | CA | ARG | B | 93 | 6.006 | 55.993 | 88.051 | 1.00 | 160.97 | 6 |
| ATOM | 2445 | CB | ARG | B | 93 | 4.510 | 55.665 | 88.086 | 1.00 | 102.93 | 6 |
| ATOM | 2446 | CG | ARG | B | 93 | 3.589 | 56.842 | 87.749 | 1.00 | 103.89 | 6 |
| ATOM | 2447 | CD | ARG | B | 93 | 2.114 | 56.512 | 88.042 | 1.00 | 104.42 | 6 |
| ATOM | 2448 | NE | ARG | B | 93 | 1.273 | 57.709 | 88.143 | 1.00 | 103.76 | 7 |
| ATOM | 2449 | CZ | ARG | B | 93 | 0.003 | 57.706 | 88.545 | 1.00 | 103.70 | 6 |
| ATOM | 2450 | NH1 | ARG | B | 93 | −0.586 | 56.568 | 88.885 | 1.00 | 103.43 | 7 |
| ATOM | 2451 | NH2 | ARG | B | 93 | −0.678 | 58.842 | 88.616 | 1.00 | 104.03 | 7 |
| ATOM | 2452 | C | ARG | B | 93 | 6.380 | 56.981 | 89.155 | 1.00 | 160.96 | 6 |
| ATOM | 2453 | O | ARG | B | 93 | 5.556 | 57.381 | 89.974 | 1.00 | 161.51 | 8 |
| ATOM | 2454 | N | TRP | B | 94 | 7.657 | 57.349 | 89.151 | 1.00 | 171.63 | 7 |
| ATOM | 2455 | CA | TRP | B | 94 | 8.276 | 58.230 | 90.139 | 1.00 | 170.47 | 6 |
| ATOM | 2456 | CB | TRP | B | 94 | 9.763 | 57.888 | 90.163 | 1.00 | 167.50 | 6 |
| ATOM | 2457 | CG | TRP | B | 94 | 10.568 | 58.559 | 91.201 | 1.00 | 170.85 | 6 |
| ATOM | 2458 | CD2 | TRP | B | 94 | 11.423 | 59.692 | 91.019 | 1.00 | 171.98 | 6 |
| ATOM | 2459 | CE2 | TRP | B | 94 | 12.024 | 59.963 | 92.262 | 1.00 | 173.23 | 6 |
| ATOM | 2460 | CE3 | TRP | B | 94 | 11.740 | 60.505 | 89.922 | 1.00 | 171.41 | 6 |
| ATOM | 2461 | CD1 | TRP | B | 94 | 10.676 | 58.200 | 92.507 | 1.00 | 172.08 | 6 |
| ATOM | 2462 | NE1 | TRP | B | 94 | 11.550 | 59.036 | 93.153 | 1.00 | 173.59 | 7 |
| ATOM | 2463 | CZ2 | TRP | B | 94 | 12.927 | 61.013 | 92.446 | 1.00 | 174.27 | 6 |
| ATOM | 2464 | CZ3 | TRP | B | 94 | 12.637 | 61.549 | 90.103 | 1.00 | 172.06 | 6 |
| ATOM | 2465 | CH2 | TRP | B | 94 | 13.220 | 61.792 | 91.356 | 1.00 | 173.80 | 6 |
| ATOM | 2466 | C | TRP | B | 94 | 8.086 | 59.748 | 89.972 | 1.00 | 168.43 | 6 |
| ATOM | 2467 | O | TRP | B | 94 | 7.625 | 60.219 | 88.929 | 1.00 | 168.12 | 8 |
| ATOM | 2468 | N | ARG | B | 95 | 8.458 | 60.489 | 91.022 | 1.00 | 108.70 | 7 |
| ATOM | 2469 | CA | ARG | B | 95 | 8.374 | 61.957 | 91.100 | 1.00 | 105.59 | 6 |
| ATOM | 2470 | CB | ARG | B | 95 | 7.074 | 62.460 | 90.462 | 1.00 | 152.49 | 6 |
| ATOM | 2471 | CG | ARG | B | 95 | 5.833 | 61.748 | 90.970 | 1.00 | 153.87 | 6 |
| ATOM | 2472 | CD | ARG | B | 95 | 4.759 | 62.732 | 91.395 | 1.00 | 154.72 | 6 |
| ATOM | 2473 | NE | ARG | B | 95 | 3.626 | 62.056 | 92.019 | 1.00 | 156.37 | 7 |
| ATOM | 2474 | CZ | ARG | B | 95 | 2.569 | 62.682 | 92.525 | 1.00 | 157.56 | 6 |
| ATOM | 2475 | NH1 | ARG | B | 95 | 2.494 | 64.007 | 92.483 | 1.00 | 158.22 | 7 |
| ATOM | 2476 | NH2 | ARG | B | 95 | 1.586 | 61.983 | 93.077 | 1.00 | 158.25 | 7 |
| ATOM | 2477 | C | ARG | B | 95 | 8.405 | 62.356 | 92.583 | 1.00 | 103.14 | 6 |
| ATOM | 2478 | O | ARG | B | 95 | 7.460 | 62.954 | 93.096 | 1.00 | 103.69 | 8 |
| ATOM | 2479 | N | THR | B | 96 | 9.509 | 62.045 | 93.258 | 1.00 | 76.25 | 7 |
| ATOM | 2480 | CA | THR | B | 96 | 9.641 | 62.303 | 94.694 | 1.00 | 73.20 | 6 |
| ATOM | 2481 | CB | THR | B | 96 | 10.349 | 61.171 | 95.379 | 1.00 | 62.79 | 6 |
| ATOM | 2482 | OG1 | THR | B | 96 | 11.738 | 61.215 | 95.027 | 1.00 | 60.49 | 8 |
| ATOM | 2483 | CG2 | THR | B | 96 | 9.727 | 59.850 | 94.975 | 1.00 | 64.35 | 6 |
| ATOM | 2484 | C | THR | B | 96 | 10.340 | 63.545 | 95.211 | 1.00 | 72.96 | 6 |
| ATOM | 2485 | O | THR | B | 96 | 10.643 | 64.476 | 94.470 | 1.00 | 73.64 | 8 |
| ATOM | 2486 | N | THR | B | 97 | 10.602 | 63.512 | 96.519 | 1.00 | 58.24 | 7 |
| ATOM | 2487 | CA | THR | B | 97 | 11.256 | 64.595 | 97.253 | 1.00 | 54.45 | 6 |
| ATOM | 2488 | CB | THR | B | 97 | 10.249 | 65.333 | 98.180 | 1.00 | 57.05 | 6 |
| ATOM | 2489 | OG1 | THR | B | 97 | 9.224 | 65.949 | 97.388 | 1.00 | 57.08 | 8 |
| ATOM | 2490 | CG2 | THR | B | 97 | 10.957 | 66.393 | 99.012 | 1.00 | 57.27 | 6 |
| ATOM | 2491 | C | THR | B | 97 | 12.391 | 64.041 | 98.118 | 1.00 | 53.16 | 6 |
| ATOM | 2492 | O | THR | B | 97 | 12.416 | 62.857 | 98.457 | 1.00 | 51.75 | 8 |
| ATOM | 2493 | N | LEU | B | 98 | 13.321 | 64.918 | 98.473 | 1.00 | 49.32 | 7 |
| ATOM | 2494 | CA | LEU | B | 98 | 14.466 | 64.560 | 99.290 | 1.00 | 48.21 | 6 |
| ATOM | 2495 | CB | LEU | B | 98 | 15.746 | 64.676 | 98.468 | 1.00 | 126.78 | 6 |
| ATOM | 2496 | CG | LEU | B | 98 | 15.672 | 64.157 | 97.032 | 1.00 | 128.47 | 6 |
| ATOM | 2497 | CD1 | LEU | B | 98 | 16.965 | 64.491 | 96.312 | 1.00 | 129.98 | 6 |
| ATOM | 2498 | CD2 | LEU | B | 98 | 15.415 | 62.658 | 97.030 | 1.00 | 129.19 | 6 |
| ATOM | 2499 | C | LEU | B | 98 | 14.526 | 65.543 | 100.440 | 1.00 | 48.83 | 6 |
| ATOM | 2500 | O | LEU | B | 98 | 14.054 | 66.669 | 100.322 | 1.00 | 47.56 | 8 |
| ATOM | 2501 | N | ILE | B | 99 | 15.107 | 65.115 | 101.551 | 1.00 | 81.01 | 7 |
| ATOM | 2502 | CA | ILE | B | 99 | 15.238 | 65.973 | 102.718 | 1.00 | 82.32 | 6 |
| ATOM | 2503 | CB | ILE | B | 99 | 14.221 | 65.573 | 103.785 | 1.00 | 105.79 | 6 |
| ATOM | 2504 | CG2 | ILE | B | 99 | 14.372 | 66.445 | 105.007 | 1.00 | 105.81 | 6 |
| ATOM | 2505 | CG1 | ILE | B | 99 | 12.813 | 65.723 | 103.205 | 1.00 | 105.76 | 6 |
| ATOM | 2506 | CD1 | ILE | B | 99 | 11.713 | 65.364 | 104.160 | 1.00 | 105.01 | 6 |
| ATOM | 2507 | C | ILE | B | 99 | 16.669 | 65.842 | 103.232 | 1.00 | 85.52 | 6 |
| ATOM | 2508 | O | ILE | B | 99 | 17.411 | 64.993 | 102.749 | 1.00 | 86.30 | 8 |
| ATOM | 2509 | N | LEU | B | 100 | 17.076 | 66.659 | 104.199 | 1.00 | 129.42 | 7 |
| ATOM | 2510 | CA | LEU | B | 100 | 18.455 | 66.571 | 104.660 | 1.00 | 130.47 | 6 |
| ATOM | 2511 | CB | LEU | B | 100 | 19.299 | 67.559 | 103.854 | 1.00 | 82.55 | 6 |
| ATOM | 2512 | CG | LEU | B | 100 | 20.786 | 67.238 | 103.702 | 1.00 | 85.11 | 6 |
| ATOM | 2513 | CD1 | LEU | B | 100 | 20.965 | 65.792 | 103.280 | 1.00 | 85.38 | 6 |
| ATOM | 2514 | CD2 | LEU | B | 100 | 21.403 | 68.169 | 102.675 | 1.00 | 86.72 | 6 |
| ATOM | 2515 | C | LEU | B | 100 | 18.701 | 66.769 | 106.159 | 1.00 | 132.38 | 6 |
| ATOM | 2516 | O | LEU | B | 100 | 17.874 | 67.342 | 106.870 | 1.00 | 132.51 | 8 |
| ATOM | 2517 | N | ALA | B | 101 | 19.856 | 66.285 | 106.619 | 1.00 | 98.92 | 7 |
| ATOM | 2518 | CA | ALA | B | 101 | 20.270 | 66.373 | 108.022 | 1.00 | 99.56 | 6 |
| ATOM | 2519 | CB | ALA | B | 101 | 20.205 | 64.989 | 108.662 | 1.00 | 57.99 | 6 |
| ATOM | 2520 | C | ALA | B | 101 | 21.694 | 66.933 | 108.119 | 1.00 | 100.36 | 6 |
| ATOM | 2521 | O | ALA | B | 101 | 22.639 | 66.340 | 107.594 | 1.00 | 100.49 | 8 |
| ATOM | 2522 | N | ALA | B | 102 | 21.852 | 68.064 | 108.803 | 1.00 | 90.68 | 7 |
| ATOM | 2523 | CA | ALA | B | 102 | 23.164 | 68.691 | 108.920 | 1.00 | 91.70 | 6 |
| ATOM | 2524 | CB | ALA | B | 102 | 23.083 | 70.128 | 108.424 | 1.00 | 114.01 | 6 |
| ATOM | 2525 | C | ALA | B | 102 | 23.796 | 68.656 | 110.312 | 1.00 | 94.60 | 6 |
| ATOM | 2526 | O | ALA | B | 102 | 23.135 | 68.904 | 111.321 | 1.00 | 95.30 | 8 |
| ATOM | 2527 | N | ALA | B | 103 | 25.095 | 68.367 | 110.342 | 1.00 | 107.85 | 7 |
| ATOM | 2528 | CA | ALA | B | 103 | 25.858 | 68.287 | 111.581 | 1.00 | 109.82 | 6 |
| ATOM | 2529 | CB | ALA | B | 103 | 26.539 | 66.929 | 111.675 | 1.00 | 162.25 | 6 |
| ATOM | 2530 | C | ALA | B | 103 | 26.900 | 69.401 | 111.696 | 1.00 | 110.77 | 6 |
| ATOM | 2531 | O | ALA | B | 103 | 26.803 | 70.426 | 111.027 | 1.00 | 111.66 | 8 |
| ATOM | 2532 | N | GLY | B | 104 | 27.903 | 69.183 | 112.542 | 1.00 | 119.02 | 7 |
| ATOM | 2533 | CA | GLY | B | 104 | 28.945 | 70.174 | 112.756 | 1.00 | 117.56 | 6 |
| ATOM | 2534 | C | GLY | B | 104 | 29.728 | 70.570 | 111.522 | 1.00 | 118.84 | 6 |
| ATOM | 2535 | O | GLY | B | 104 | 29.191 | 70.570 | 110.414 | 1.00 | 118.81 | 8 |
| ATOM | 2536 | N | PRO | B | 105 | 31.018 | 70.901 | 111.685 | 1.00 | 112.08 | 7 |
| ATOM | 2537 | CD | PRO | B | 105 | 31.762 | 70.769 | 112.949 | 1.00 | 147.49 | 6 |
| ATOM | 2538 | CA | PRO | B | 105 | 31.913 | 71.313 | 110.596 | 1.00 | 111.87 | 6 |
| ATOM | 2539 | CB | PRO | B | 105 | 33.275 | 71.415 | 111.290 | 1.00 | 147.97 | 6 |
| ATOM | 2540 | CG | PRO | B | 105 | 33.148 | 70.476 | 112.457 | 1.00 | 147.73 | 6 |
| ATOM | 2541 | C | PRO | B | 105 | 31.959 | 70.448 | 109.326 | 1.00 | 112.31 | 6 |
| ATOM | 2542 | O | PRO | B | 105 | 32.227 | 70.955 | 108.238 | 1.00 | 112.30 | 8 |
| ATOM | 2543 | N | LYS | B | 106 | 31.690 | 69.155 | 109.466 | 1.00 | 131.56 | 7 |
| ATOM | 2544 | CA | LYS | B | 106 | 31.717 | 68.219 | 108.341 | 1.00 | 130.72 | 6 |
| ATOM | 2545 | CB | LYS | B | 106 | 31.027 | 66.908 | 108.742 | 1.00 | 199.00 | 6 |
| ATOM | 2546 | CG | LYS | B | 106 | 31.550 | 66.288 | 110.037 | 1.00 | 199.67 | 6 |
| ATOM | 2547 | CD | LYS | B | 106 | 33.040 | 65.977 | 109.952 | 1.00 | 200.50 | 6 |
| ATOM | 2548 | CE | LYS | B | 106 | 33.571 | 65.438 | 111.273 | 1.00 | 201.17 | 6 |
| ATOM | 2549 | NZ | LYS | B | 106 | 35.043 | 65.192 | 111.248 | 1.00 | 201.26 | 7 |
| ATOM | 2550 | C | LYS | B | 106 | 31.080 | 68.750 | 107.053 | 1.00 | 130.11 | 6 |
| ATOM | 2551 | O | LYS | B | 106 | 30.327 | 69.728 | 107.072 | 1.00 | 130.08 | 8 |
| ATOM | 2552 | N | GLU | B | 107 | 31.398 | 68.090 | 105.937 | 1.00 | 46.75 | 7 |
| ATOM | 2553 | CA | GLU | B | 107 | 30.868 | 68.449 | 104.625 | 1.00 | 45.13 | 6 |
| ATOM | 2554 | CB | GLU | B | 107 | 31.787 | 67.913 | 103.527 | 1.00 | 152.42 | 6 |
| ATOM | 2555 | CG | GLU | B | 107 | 33.188 | 68.481 | 103.569 | 1.00 | 155.40 | 6 |
| ATOM | 2556 | CD | GLU | B | 107 | 34.072 | 67.928 | 102.471 | 1.00 | 158.04 | 6 |
| ATOM | 2557 | OE1 | GLU | B | 107 | 33.693 | 68.031 | 101.286 | 1.00 | 159.56 | 8 |
| ATOM | 2558 | OE2 | GLU | B | 107 | 35.153 | 67.393 | 102.789 | 1.00 | 159.19 | 8 |
| ATOM | 2559 | C | GLU | B | 107 | 29.460 | 67.877 | 104.430 | 1.00 | 44.32 | 6 |
| ATOM | 2560 | O | GLU | B | 107 | 29.110 | 66.857 | 105.012 | 1.00 | 43.92 | 8 |
| ATOM | 2561 | N | VAL | B | 108 | 28.640 | 68.544 | 103.627 | 1.00 | 79.93 | 7 |
| ATOM | 2562 | CA | VAL | B | 108 | 27.296 | 68.044 | 103.365 | 1.00 | 79.62 | 6 |
| ATOM | 2563 | CB | VAL | B | 108 | 26.192 | 69.146 | 103.527 | 1.00 | 52.82 | 6 |
| ATOM | 2564 | CG1 | VAL | B | 108 | 24.827 | 68.584 | 103.184 | 1.00 | 51.88 | 6 |
| ATOM | 2565 | CG2 | VAL | B | 108 | 26.154 | 69.646 | 104.955 | 1.00 | 54.40 | 6 |
| ATOM | 2566 | C | VAL | B | 108 | 27.309 | 67.539 | 101.933 | 1.00 | 79.72 | 6 |
| ATOM | 2567 | O | VAL | B | 108 | 26.995 | 68.269 | 100.997 | 1.00 | 81.03 | 8 |
| ATOM | 2568 | N | ARG | B | 109 | 27.712 | 66.286 | 101.773 | 1.00 | 72.39 | 7 |
| ATOM | 2569 | CA | ARG | B | 109 | 27.761 | 65.661 | 100.462 | 1.00 | 71.53 | 6 |
| ATOM | 2570 | CB | ARG | B | 109 | 28.515 | 64.336 | 100.543 | 1.00 | 75.22 | 6 |
| ATOM | 2571 | CG | ARG | B | 109 | 29.606 | 64.308 | 101.590 | 1.00 | 74.93 | 6 |
| ATOM | 2572 | CD | ARG | B | 109 | 30.934 | 63.925 | 100.968 | 1.00 | 74.31 | 6 |
| ATOM | 2573 | NE | ARG | B | 109 | 32.020 | 63.961 | 101.939 | 1.00 | 74.49 | 7 |
| ATOM | 2574 | CZ | ARG | B | 109 | 33.268 | 64.308 | 101.646 | 1.00 | 75.44 | 6 |
| ATOM | 2575 | NH1 | ARG | B | 109 | 33.589 | 64.654 | 100.404 | 1.00 | 75.90 | 7 |
| ATOM | 2576 | NH2 | ARG | B | 109 | 34.193 | 64.310 | 102.595 | 1.00 | 76.18 | 7 |
| ATOM | 2577 | C | ARG | B | 109 | 26.331 | 65.381 | 100.012 | 1.00 | 71.79 | 6 |
| ATOM | 2578 | O | ARG | B | 109 | 25.411 | 65.321 | 100.834 | 1.00 | 72.18 | 8 |
| ATOM | 2579 | N | ALA | B | 110 | 26.137 | 65.199 | 98.712 | 1.00 | 60.56 | 7 |
| ATOM | 2580 | CA | ALA | B | 110 | 24.809 | 64.890 | 98.204 | 1.00 | 60.62 | 6 |
| ATOM | 2581 | CB | ALA | B | 110 | 24.823 | 64.883 | 96.680 | 1.00 | 116.89 | 6 |
| ATOM | 2582 | C | ALA | B | 110 | 24.408 | 63.511 | 98.745 | 1.00 | 60.99 | 6 |
| ATOM | 2583 | O | ALA | B | 110 | 23.337 | 62.990 | 98.436 | 1.00 | 60.39 | 8 |
| ATOM | 2584 | N | VAL | B | 111 | 25.287 | 62.928 | 99.556 | 1.00 | 115.68 | 7 |
| ATOM | 2585 | CA | VAL | B | 111 | 25.054 | 61.617 | 100.152 | 1.00 | 116.53 | 6 |
| ATOM | 2586 | CB | VAL | B | 111 | 26.381 | 60.972 | 100.638 | 1.00 | 171.54 | 6 |
| ATOM | 2587 | CG1 | VAL | B | 111 | 26.136 | 59.533 | 101.086 | 1.00 | 171.02 | 6 |
| ATOM | 2588 | CG2 | VAL | B | 111 | 27.422 | 61.017 | 99.534 | 1.00 | 172.39 | 6 |
| ATOM | 2589 | C | VAL | B | 111 | 24.117 | 61.739 | 101.348 | 1.00 | 116.52 | 6 |
| ATOM | 2590 | O | VAL | B | 111 | 23.121 | 61.021 | 101.446 | 1.00 | 116.08 | 8 |
| ATOM | 2591 | N | ASP | B | 112 | 24.446 | 62.653 | 102.257 | 1.00 | 93.32 | 7 |
| ATOM | 2592 | CA | ASP | B | 112 | 23.649 | 62.875 | 103.458 | 1.00 | 93.72 | 6 |
| ATOM | 2593 | CB | ASP | B | 112 | 23.949 | 64.258 | 104.039 | 1.00 | 208.87 | 6 |
| ATOM | 2594 | CG | ASP | B | 112 | 25.386 | 64.397 | 104.498 | 1.00 | 208.87 | 6 |
| ATOM | 2595 | OD1 | ASP | B | 112 | 25.746 | 65.487 | 104.988 | 1.00 | 208.87 | 8 |
| ATOM | 2596 | OD2 | ASP | B | 112 | 26.154 | 63.420 | 104.374 | 1.00 | 208.87 | 8 |
| ATOM | 2597 | C | ASP | B | 112 | 22.161 | 62.754 | 103.174 | 1.00 | 93.88 | 6 |
| ATOM | 2598 | O | ASP | B | 112 | 21.403 | 62.243 | 104.000 | 1.00 | 93.49 | 8 |
| ATOM | 2599 | N | PHE | B | 113 | 21.754 | 63.223 | 101.997 | 1.00 | 140.32 | 7 |
| ATOM | 2600 | CA | PHE | B | 113 | 20.357 | 63.175 | 101.587 | 1.00 | 139.27 | 6 |
| ATOM | 2601 | CB | PHE | B | 113 | 20.239 | 63.102 | 100.059 | 1.00 | 88.94 | 6 |
| ATOM | 2602 | CG | PHE | B | 113 | 20.398 | 64.431 | 99.369 | 1.00 | 88.94 | 6 |
| ATOM | 2603 | CD1 | PHE | B | 113 | 21.643 | 65.049 | 99.284 | 1.00 | 89.09 | 6 |
| ATOM | 2604 | CD2 | PHE | B | 113 | 19.295 | 65.069 | 98.804 | 1.00 | 88.14 | 6 |
| ATOM | 2605 | CE1 | PHE | B | 113 | 21.792 | 66.285 | 98.645 | 1.00 | 89.50 | 6 |
| ATOM | 2606 | CE2 | PHE | B | 113 | 19.429 | 66.305 | 98.164 | 1.00 | 88.22 | 6 |
| ATOM | 2607 | CZ | PHE | B | 113 | 20.685 | 66.915 | 98.084 | 1.00 | 89.00 | 6 |
| ATOM | 2608 | C | PHE | B | 113 | 19.639 | 61.984 | 102.199 | 1.00 | 139.39 | 6 |
| ATOM | 2609 | O | PHE | B | 113 | 20.129 | 60.859 | 102.125 | 1.00 | 139.80 | 8 |
| ATOM | 2610 | N | THR | B | 114 | 18.491 | 62.257 | 102.819 | 1.00 | 89.09 | 7 |
| ATOM | 2611 | CA | THR | B | 114 | 17.644 | 61.240 | 103.444 | 1.00 | 88.38 | 6 |
| ATOM | 2612 | CB | THR | B | 114 | 16.190 | 61.743 | 103.574 | 1.00 | 160.36 | 6 |
| ATOM | 2613 | OG1 | THR | B | 114 | 16.151 | 62.882 | 104.444 | 1.00 | 160.56 | 8 |
| ATOM | 2614 | CG2 | THR | B | 114 | 15.290 | 60.644 | 104.121 | 1.00 | 160.45 | 6 |
| ATOM | 2615 | C | THR | B | 114 | 17.647 | 59.997 | 102.566 | 1.00 | 88.16 | 6 |
| ATOM | 2616 | O | THR | B | 114 | 16.781 | 59.844 | 101.699 | 1.00 | 87.90 | 8 |
| ATOM | 2617 | N | PRO | B | 115 | 18.610 | 59.086 | 102.802 | 1.00 | 71.69 | 7 |
| ATOM | 2618 | CD | PRO | B | 115 | 19.412 | 59.070 | 104.038 | 1.00 | 97.44 | 6 |
| ATOM | 2619 | CA | PRO | B | 115 | 18.791 | 57.832 | 102.060 | 1.00 | 71.19 | 6 |
| ATOM | 2620 | CB | PRO | B | 115 | 19.749 | 57.035 | 102.953 | 1.00 | 97.50 | 6 |
| ATOM | 2621 | CG | PRO | B | 115 | 19.505 | 57.603 | 104.325 | 1.00 | 97.83 | 6 |
| ATOM | 2622 | C | PRO | B | 115 | 17.526 | 57.059 | 101.704 | 1.00 | 71.53 | 6 |
| ATOM | 2623 | O | PRO | B | 115 | 17.328 | 55.931 | 102.147 | 1.00 | 71.33 | 8 |
| ATOM | 2624 | N | SER | B | 116 | 16.683 | 57.677 | 100.884 | 1.00 | 146.16 | 7 |
| ATOM | 2625 | CA | SER | B | 116 | 15.444 | 57.059 | 100.444 | 1.00 | 146.33 | 6 |
| ATOM | 2626 | CB | SER | B | 116 | 14.477 | 58.129 | 99.921 | 1.00 | 82.24 | 6 |
| ATOM | 2627 | OG | SER | B | 116 | 13.264 | 57.556 | 99.459 | 1.00 | 80.53 | 8 |
| ATOM | 2628 | C | SER | B | 116 | 15.770 | 56.058 | 99.341 | 1.00 | 147.50 | 6 |
| ATOM | 2629 | O | SER | B | 116 | 16.562 | 56.347 | 98.442 | 1.00 | 147.96 | 8 |
| ATOM | 2630 | N | ALA | B | 117 | 15.175 | 54.873 | 99.423 | 1.00 | 154.86 | 7 |
| ATOM | 2631 | CA | ALA | B | 117 | 15.407 | 53.841 | 98.422 | 1.00 | 154.61 | 6 |
| ATOM | 2632 | CB | ALA | B | 117 | 14.884 | 52.505 | 98.920 | 1.00 | 152.66 | 6 |
| ATOM | 2633 | C | ALA | B | 117 | 14.695 | 54.240 | 97.141 | 1.00 | 155.24 | 6 |
| ATOM | 2634 | O | ALA | B | 117 | 14.167 | 55.347 | 97.042 | 1.00 | 155.74 | 8 |
| ATOM | 2635 | N | ASP | B | 118 | 14.688 | 53.339 | 96.166 | 1.00 | 88.55 | 7 |
| ATOM | 2636 | CA | ASP | B | 118 | 14.037 | 53.588 | 94.887 | 1.00 | 88.64 | 6 |
| ATOM | 2637 | CB | ASP | B | 118 | 12.510 | 53.457 | 95.026 | 1.00 | 118.28 | 6 |
| ATOM | 2638 | CG | ASP | B | 118 | 11.902 | 54.515 | 95.930 | 1.00 | 119.07 | 6 |
| ATOM | 2639 | OD1 | ASP | B | 118 | 11.985 | 54.381 | 97.170 | 1.00 | 119.87 | 8 |
| ATOM | 2640 | OD2 | ASP | B | 118 | 11.341 | 55.489 | 95.389 | 1.00 | 119.11 | 8 |
| ATOM | 2641 | C | ASP | B | 118 | 14.385 | 54.950 | 94.293 | 1.00 | 88.12 | 6 |
| ATOM | 2642 | O | ASP | B | 118 | 13.772 | 55.391 | 93.323 | 1.00 | 87.96 | 8 |
| ATOM | 2643 | N | VAL | B | 119 | 15.382 | 55.603 | 94.882 | 1.00 | 77.20 | 7 |
| ATOM | 2644 | CA | VAL | B | 119 | 15.845 | 56.922 | 94.460 | 1.00 | 77.42 | 6 |
| ATOM | 2645 | CB | VAL | B | 119 | 14.940 | 58.045 | 95.043 | 1.00 | 51.81 | 6 |
| ATOM | 2646 | CG1 | VAL | B | 119 | 15.413 | 59.407 | 94.585 | 1.00 | 51.41 | 6 |
| ATOM | 2647 | CG2 | VAL | B | 119 | 13.512 | 57.829 | 94.616 | 1.00 | 51.98 | 6 |
| ATOM | 2648 | C | VAL | B | 119 | 17.258 | 57.079 | 95.018 | 1.00 | 77.32 | 6 |
| ATOM | 2649 | O | VAL | B | 119 | 17.451 | 57.666 | 96.085 | 1.00 | 77.65 | 8 |
| ATOM | 2650 | N | GLU | B | 120 | 18.244 | 56.535 | 94.314 | 1.00 | 142.78 | 7 |
| ATOM | 2651 | CA | GLU | B | 120 | 19.624 | 56.634 | 94.775 | 1.00 | 142.83 | 6 |
| ATOM | 2652 | CB | GLU | B | 120 | 20.325 | 55.271 | 94.669 | 1.00 | 114.74 | 6 |
| ATOM | 2653 | CG | GLU | B | 120 | 21.680 | 55.207 | 95.375 | 1.00 | 115.85 | 6 |
| ATOM | 2654 | CD | GLU | B | 120 | 22.377 | 53.868 | 95.205 | 1.00 | 117.05 | 6 |
| ATOM | 2655 | OE1 | GLU | B | 120 | 22.751 | 53.520 | 94.063 | 1.00 | 117.57 | 8 |
| ATOM | 2656 | OE2 | GLU | B | 120 | 22.546 | 53.160 | 96.217 | 1.00 | 117.15 | 8 |
| ATOM | 2657 | C | GLU | B | 120 | 20.398 | 57.684 | 93.979 | 1.00 | 143.11 | 6 |
| ATOM | 2658 | O | GLU | B | 120 | 20.442 | 57.645 | 92.746 | 1.00 | 143.41 | 8 |
| ATOM | 2659 | N | ILE | B | 121 | 20.991 | 58.631 | 94.699 | 1.00 | 89.19 | 7 |
| ATOM | 2660 | CA | ILE | B | 121 | 21.776 | 59.686 | 94.083 | 1.00 | 87.07 | 6 |
| ATOM | 2661 | CB | ILE | B | 121 | 22.254 | 60.715 | 95.143 | 1.00 | 137.58 | 6 |
| ATOM | 2662 | CG2 | ILE | B | 121 | 23.420 | 61.516 | 94.608 | 1.00 | 138.22 | 6 |
| ATOM | 2663 | CG1 | ILE | B | 121 | 21.112 | 61.665 | 95.515 | 1.00 | 137.41 | 6 |
| ATOM | 2664 | CD1 | ILE | B | 121 | 19.941 | 61.000 | 96.191 | 1.00 | 137.99 | 6 |
| ATOM | 2665 | C | ILE | B | 121 | 22.980 | 59.075 | 93.365 | 1.00 | 86.78 | 6 |
| ATOM | 2666 | O | ILE | B | 121 | 23.945 | 58.627 | 93.996 | 1.00 | 85.86 | 8 |
| ATOM | 2667 | N | MET | B | 122 | 22.883 | 59.058 | 92.037 | 1.00 | 88.09 | 7 |
| ATOM | 2668 | CA | MET | B | 122 | 23.902 | 58.529 | 91.131 | 1.00 | 86.88 | 6 |
| ATOM | 2669 | CB | MET | B | 122 | 23.585 | 58.993 | 89.709 | 1.00 | 98.50 | 6 |
| ATOM | 2670 | CG | MET | B | 122 | 23.708 | 57.940 | 88.638 | 1.00 | 100.12 | 6 |
| ATOM | 2671 | SD | MET | B | 122 | 22.126 | 57.147 | 88.335 | 1.00 | 102.51 | 16 |
| ATOM | 2672 | CE | MET | B | 122 | 22.120 | 55.932 | 89.664 | 1.00 | 103.27 | 6 |
| ATOM | 2673 | C | MET | B | 122 | 25.310 | 59.009 | 91.489 | 1.00 | 86.12 | 6 |
| ATOM | 2674 | O | MET | B | 122 | 26.288 | 58.265 | 91.379 | 1.00 | 86.13 | 8 |
| ATOM | 2675 | N | ASN | B | 123 | 25.383 | 60.273 | 91.896 | 1.00 | 102.30 | 7 |
| ATOM | 2676 | CA | ASN | B | 123 | 26.628 | 60.944 | 92.259 | 1.00 | 100.44 | 6 |
| ATOM | 2677 | CB | ASN | B | 123 | 26.719 | 62.251 | 91.479 | 1.00 | 44.03 | 6 |
| ATOM | 2678 | CG | ASN | B | 123 | 25.395 | 62.996 | 91.442 | 1.00 | 43.06 | 6 |
| ATOM | 2679 | OD1 | ASN | B | 123 | 24.329 | 62.407 | 91.626 | 1.00 | 41.66 | 8 |
| ATOM | 2680 | ND2 | ASN | B | 123 | 25.457 | 64.299 | 91.184 | 1.00 | 42.20 | 7 |
| ATOM | 2681 | C | ASN | B | 123 | 26.728 | 61.212 | 93.763 | 1.00 | 100.01 | 6 |
| ATOM | 2682 | O | ASN | B | 123 | 26.158 | 62.172 | 94.278 | 1.00 | 100.97 | 8 |
| ATOM | 2683 | N | PRO | B | 124 | 27.492 | 60.375 | 94.479 | 1.00 | 40.54 | 7 |
| ATOM | 2684 | CD | PRO | B | 124 | 28.486 | 59.467 | 93.880 | 1.00 | 130.63 | 6 |
| ATOM | 2685 | CA | PRO | B | 124 | 27.699 | 60.466 | 95.923 | 1.00 | 38.86 | 6 |
| ATOM | 2686 | CB | PRO | B | 124 | 28.775 | 59.419 | 96.181 | 1.00 | 129.78 | 6 |
| ATOM | 2687 | CG | PRO | B | 124 | 29.578 | 59.461 | 94.923 | 1.00 | 130.60 | 6 |
| ATOM | 2688 | C | PRO | B | 124 | 28.124 | 61.831 | 96.413 | 1.00 | 38.04 | 6 |
| ATOM | 2689 | O | PRO | B | 124 | 27.353 | 62.534 | 97.059 | 1.00 | 37.31 | 8 |
| ATOM | 2690 | N | ASP | B | 125 | 29.361 | 62.191 | 96.100 | 1.00 | 96.40 | 7 |
| ATOM | 2691 | CA | ASP | B | 125 | 29.952 | 63.453 | 96.517 | 1.00 | 97.07 | 6 |
| ATOM | 2692 | CB | ASP | B | 125 | 31.336 | 63.556 | 95.900 | 1.00 | 90.27 | 6 |
| ATOM | 2693 | CG | ASP | B | 125 | 32.054 | 62.228 | 95.911 | 1.00 | 89.52 | 6 |
| ATOM | 2694 | OD1 | ASP | B | 125 | 32.136 | 61.608 | 96.992 | 1.00 | 88.48 | 8 |
| ATOM | 2695 | OD2 | ASP | B | 125 | 32.526 | 61.797 | 94.841 | 1.00 | 90.20 | 8 |
| ATOM | 2696 | C | ASP | B | 125 | 29.129 | 64.698 | 96.201 | 1.00 | 97.77 | 6 |
| ATOM | 2697 | O | ASP | B | 125 | 27.976 | 64.809 | 96.623 | 1.00 | 98.95 | 8 |
| ATOM | 2698 | N | LEU | B | 126 | 29.725 | 65.640 | 95.473 | 1.00 | 65.34 | 7 |
| ATOM | 2699 | CA | LEU | B | 126 | 29.038 | 66.882 | 95.114 | 1.00 | 64.52 | 6 |
| ATOM | 2700 | CB | LEU | B | 126 | 27.659 | 66.584 | 94.511 | 1.00 | 106.19 | 6 |
| ATOM | 2701 | CG | LEU | B | 126 | 26.828 | 67.776 | 94.031 | 1.00 | 106.27 | 6 |
| ATOM | 2702 | CD1 | LEU | B | 126 | 27.577 | 68.543 | 92.956 | 1.00 | 106.51 | 6 |
| ATOM | 2703 | CD2 | LEU | B | 126 | 25.506 | 67.274 | 93.489 | 1.00 | 106.74 | 6 |
| ATOM | 2704 | C | LEU | B | 126 | 28.884 | 67.736 | 96.363 | 1.00 | 64.64 | 6 |
| ATOM | 2705 | O | LEU | B | 126 | 27.928 | 67.584 | 97.121 | 1.00 | 65.05 | 8 |
| ATOM | 2706 | N | HIS | B | 127 | 29.831 | 68.641 | 96.558 | 1.00 | 66.42 | 7 |
| ATOM | 2707 | CA | HIS | B | 127 | 29.862 | 69.514 | 97.719 | 1.00 | 65.85 | 6 |
| ATOM | 2708 | CB | HIS | B | 127 | 30.625 | 70.790 | 97.376 | 1.00 | 101.20 | 6 |
| ATOM | 2709 | CG | HIS | B | 127 | 31.028 | 71.577 | 98.579 | 1.00 | 103.06 | 6 |
| ATOM | 2710 | CD2 | HIS | B | 127 | 32.243 | 71.799 | 99.130 | 1.00 | 104.05 | 6 |
| ATOM | 2711 | ND1 | HIS | B | 127 | 30.111 | 72.177 | 99.413 | 1.00 | 103.81 | 7 |
| ATOM | 2712 | CE1 | HIS | B | 127 | 30.746 | 72.732 | 100.429 | 1.00 | 103.99 | 6 |
| ATOM | 2713 | NE2 | HIS | B | 127 | 32.040 | 72.517 | 100.282 | 1.00 | 104.05 | 7 |
| ATOM | 2714 | C | HIS | B | 127 | 28.545 | 69.893 | 98.417 | 1.00 | 66.02 | 6 |
| ATOM | 2715 | O | HIS | B | 127 | 28.417 | 69.719 | 99.633 | 1.00 | 65.62 | 8 |
| ATOM | 2716 | N | ILE | B | 128 | 27.581 | 70.421 | 97.666 | 1.00 | 79.55 | 7 |
| ATOM | 2717 | CA | ILE | B | 128 | 26.292 | 70.848 | 98.230 | 1.00 | 79.18 | 6 |
| ATOM | 2718 | CB | ILE | B | 128 | 25.625 | 69.750 | 99.095 | 1.00 | 63.78 | 6 |
| ATOM | 2719 | CG2 | ILE | B | 128 | 24.424 | 70.340 | 99.820 | 1.00 | 62.99 | 6 |
| ATOM | 2720 | CG1 | ILE | B | 128 | 25.170 | 68.571 | 98.231 | 1.00 | 65.78 | 6 |
| ATOM | 2721 | CD1 | ILE | B | 128 | 23.850 | 68.808 | 97.493 | 1.00 | 68.57 | 6 |
| ATOM | 2722 | C | ILE | B | 128 | 26.410 | 72.102 | 99.104 | 1.00 | 79.47 | 6 |
| ATOM | 2723 | O | ILE | B | 128 | 25.657 | 73.057 | 98.916 | 1.00 | 80.04 | 8 |
| ATOM | 2724 | N | ALA | B | 129 | 27.339 | 72.084 | 100.062 | 1.00 | 55.47 | 7 |
| ATOM | 2725 | CA | ALA | B | 129 | 27.575 | 73.206 | 100.980 | 1.00 | 54.86 | 6 |
| ATOM | 2726 | CB | ALA | B | 129 | 26.259 | 73.847 | 101.405 | 1.00 | 23.67 | 6 |
| ATOM | 2727 | C | ALA | B | 129 | 28.325 | 72.717 | 102.216 | 1.00 | 55.53 | 6 |
| ATOM | 2728 | O | ALA | B | 129 | 28.192 | 71.562 | 102.614 | 1.00 | 56.09 | 8 |
| ATOM | 2729 | N | THR | B | 130 | 29.114 | 73.607 | 102.814 | 1.00 | 50.17 | 7 |
| ATOM | 2730 | CA | THR | B | 130 | 29.904 | 73.287 | 104.000 | 1.00 | 50.46 | 6 |
| ATOM | 2731 | CB | THR | B | 130 | 31.407 | 73.620 | 103.808 | 1.00 | 75.42 | 6 |
| ATOM | 2732 | OG1 | THR | B | 130 | 32.044 | 72.594 | 103.034 | 1.00 | 75.20 | 8 |
| ATOM | 2733 | CG2 | THR | B | 130 | 32.099 | 73.726 | 105.145 | 1.00 | 75.41 | 6 |
| ATOM | 2734 | C | THR | B | 130 | 29.390 | 74.121 | 105.139 | 1.00 | 51.78 | 6 |
| ATOM | 2735 | O | THR | B | 130 | 29.106 | 75.296 | 104.958 | 1.00 | 50.09 | 8 |
| ATOM | 2736 | N | LEU | B | 131 | 29.293 | 73.503 | 106.312 | 1.00 | 126.03 | 7 |
| ATOM | 2737 | CA | LEU | B | 131 | 28.801 | 74.154 | 107.525 | 1.00 | 127.64 | 6 |
| ATOM | 2738 | CB | LEU | B | 131 | 27.337 | 73.735 | 107.756 | 1.00 | 64.76 | 6 |
| ATOM | 2739 | CG | LEU | B | 131 | 26.947 | 72.261 | 107.965 | 1.00 | 64.71 | 6 |
| ATOM | 2740 | CD1 | LEU | B | 131 | 25.510 | 72.063 | 107.494 | 1.00 | 64.74 | 6 |
| ATOM | 2741 | CD2 | LEU | B | 131 | 27.878 | 71.331 | 107.197 | 1.00 | 65.16 | 6 |
| ATOM | 2742 | C | LEU | B | 131 | 29.693 | 73.740 | 108.704 | 1.00 | 129.43 | 6 |
| ATOM | 2743 | O | LEU | B | 131 | 30.266 | 72.653 | 108.677 | 1.00 | 128.82 | 8 |
| ATOM | 2744 | N | GLU | B | 132 | 29.822 | 74.586 | 109.729 | 1.00 | 97.60 | 7 |
| ATOM | 2745 | CA | GLU | B | 132 | 30.684 | 74.235 | 110.861 | 1.00 | 99.00 | 6 |
| ATOM | 2746 | CB | GLU | B | 132 | 31.951 | 75.105 | 110.828 | 1.00 | 134.43 | 6 |
| ATOM | 2747 | CG | GLU | B | 132 | 33.013 | 74.695 | 111.847 | 1.00 | 136.66 | 6 |
| ATOM | 2748 | CD | GLU | B | 132 | 34.383 | 75.287 | 111.558 | 1.00 | 137.83 | 6 |
| ATOM | 2749 | OE1 | GLU | B | 132 | 34.957 | 74.968 | 110.494 | 1.00 | 137.78 | 8 |
| ATOM | 2750 | OE2 | GLU | B | 132 | 34.884 | 76.069 | 112.394 | 1.00 | 138.17 | 8 |
| ATOM | 2751 | C | GLU | B | 132 | 30.079 | 74.236 | 112.284 | 1.00 | 99.83 | 6 |
| ATOM | 2752 | O | GLU | B | 132 | 29.306 | 73.337 | 112.638 | 1.00 | 99.70 | 8 |
| ATOM | 2753 | N | GLU | B | 133 | 30.453 | 75.226 | 113.099 | 1.00 | 95.71 | 7 |
| ATOM | 2754 | CA | GLU | B | 133 | 29.981 | 75.354 | 114.488 | 1.00 | 95.88 | 6 |
| ATOM | 2755 | CB | GLU | B | 133 | 30.861 | 76.365 | 115.246 | 1.00 | 81.48 | 6 |
| ATOM | 2756 | CG | GLU | B | 133 | 30.730 | 76.360 | 116.784 | 1.00 | 82.68 | 6 |
| ATOM | 2757 | CD | GLU | B | 133 | 31.455 | 77.541 | 117.459 | 1.00 | 82.94 | 6 |
| ATOM | 2758 | OE1 | GLU | B | 133 | 30.925 | 78.675 | 117.431 | 1.00 | 83.01 | 8 |
| ATOM | 2759 | OE2 | GLU | B | 133 | 32.561 | 77.340 | 118.011 | 1.00 | 81.98 | 8 |
| ATOM | 2760 | C | GLU | B | 133 | 28.519 | 75.797 | 114.553 | 1.00 | 96.07 | 6 |
| ATOM | 2761 | O | GLU | B | 133 | 28.080 | 76.390 | 115.537 | 1.00 | 95.61 | 8 |
| ATOM | 2762 | N | GLY | B | 134 | 27.776 | 75.505 | 113.491 | 1.00 | 119.75 | 7 |
| ATOM | 2763 | CA | GLY | B | 134 | 26.371 | 75.863 | 113.416 | 1.00 | 119.61 | 6 |
| ATOM | 2764 | C | GLY | B | 134 | 25.701 | 74.923 | 112.436 | 1.00 | 120.73 | 6 |
| ATOM | 2765 | O | GLY | B | 134 | 25.064 | 75.351 | 111.471 | 1.00 | 120.38 | 8 |
| ATOM | 2766 | N | GLY | B | 135 | 25.865 | 73.629 | 112.703 | 1.00 | 112.87 | 7 |
| ATOM | 2767 | CA | GLY | B | 135 | 25.314 | 72.589 | 111.853 | 1.00 | 112.15 | 6 |
| ATOM | 2768 | C | GLY | B | 135 | 23.805 | 72.511 | 111.756 | 1.00 | 112.82 | 6 |
| ATOM | 2769 | O | GLY | B | 135 | 23.200 | 71.521 | 112.175 | 1.00 | 113.01 | 8 |
| ATOM | 2770 | N | LYS | B | 136 | 23.203 | 73.555 | 111.191 | 1.00 | 208.87 | 7 |
| ATOM | 2771 | CA | LYS | B | 136 | 21.757 | 73.613 | 111.015 | 1.00 | 208.87 | 6 |
| ATOM | 2772 | CB | LYS | B | 136 | 21.260 | 75.057 | 111.134 | 1.00 | 117.57 | 6 |
| ATOM | 2773 | CG | LYS | B | 136 | 19.750 | 75.165 | 111.274 | 1.00 | 118.63 | 6 |
| ATOM | 2774 | CD | LYS | B | 136 | 19.347 | 76.462 | 111.953 | 1.00 | 119.24 | 6 |
| ATOM | 2775 | CE | LYS | B | 136 | 17.892 | 76.431 | 112.384 | 1.00 | 118.87 | 6 |
| ATOM | 2776 | NZ | LYS | B | 136 | 17.576 | 77.595 | 113.252 | 1.00 | 118.88 | 7 |
| ATOM | 2777 | C | LYS | B | 136 | 21.410 | 73.054 | 109.641 | 1.00 | 208.87 | 6 |
| ATOM | 2778 | O | LYS | B | 136 | 21.233 | 71.847 | 109.488 | 1.00 | 208.87 | 8 |
| ATOM | 2779 | N | LEU | B | 137 | 21.312 | 73.935 | 108.650 | 1.00 | 75.67 | 7 |
| ATOM | 2780 | CA | LEU | B | 137 | 21.010 | 73.521 | 107.281 | 1.00 | 72.95 | 6 |
| ATOM | 2781 | CB | LEU | B | 137 | 22.300 | 73.140 | 106.549 | 1.00 | 114.50 | 6 |
| ATOM | 2782 | CG | LEU | B | 137 | 22.145 | 72.433 | 105.196 | 1.00 | 114.45 | 6 |
| ATOM | 2783 | CD1 | LEU | B | 137 | 21.497 | 73.373 | 104.185 | 1.00 | 114.64 | 6 |
| ATOM | 2784 | CD2 | LEU | B | 137 | 23.512 | 71.969 | 104.707 | 1.00 | 114.47 | 6 |
| ATOM | 2785 | C | LEU | B | 137 | 20.047 | 72.353 | 107.172 | 1.00 | 72.27 | 6 |
| ATOM | 2786 | O | LEU | B | 137 | 20.452 | 71.195 | 107.264 | 1.00 | 71.68 | 8 |
| ATOM | 2787 | N | TYR | B | 138 | 18.774 | 72.660 | 106.979 | 1.00 | 99.33 | 7 |
| ATOM | 2788 | CA | TYR | B | 138 | 17.782 | 71.615 | 106.813 | 1.00 | 98.44 | 6 |
| ATOM | 2789 | CB | TYR | B | 138 | 16.705 | 71.682 | 107.883 | 1.00 | 101.24 | 6 |
| ATOM | 2790 | CG | TYR | B | 138 | 15.626 | 70.644 | 107.666 | 1.00 | 103.67 | 6 |
| ATOM | 2791 | CD1 | TYR | B | 138 | 15.901 | 69.283 | 107.810 | 1.00 | 105.20 | 6 |
| ATOM | 2792 | CE1 | TYR | B | 138 | 14.911 | 68.321 | 107.586 | 1.00 | 105.71 | 6 |
| ATOM | 2793 | CD2 | TYR | B | 138 | 14.331 | 71.020 | 107.291 | 1.00 | 104.41 | 6 |
| ATOM | 2794 | CE2 | TYR | B | 138 | 13.334 | 70.065 | 107.063 | 1.00 | 104.53 | 6 |
| ATOM | 2795 | CZ | TYR | B | 138 | 13.632 | 68.720 | 107.213 | 1.00 | 105.23 | 6 |
| ATOM | 2796 | OH | TYR | B | 138 | 12.654 | 67.781 | 106.988 | 1.00 | 105.25 | 8 |
| ATOM | 2797 | C | TYR | B | 138 | 17.140 | 71.823 | 105.461 | 1.00 | 97.46 | 6 |
| ATOM | 2798 | O | TYR | B | 138 | 16.706 | 72.930 | 105.141 | 1.00 | 98.49 | 8 |
| ATOM | 2799 | N | MET | B | 139 | 17.071 | 70.760 | 104.668 | 1.00 | 55.68 | 7 |
| ATOM | 2800 | CA | MET | B | 139 | 16.488 | 70.870 | 103.343 | 1.00 | 53.23 | 6 |
| ATOM | 2801 | CB | MET | B | 139 | 17.575 | 70.922 | 102.276 | 1.00 | 45.97 | 6 |
| ATOM | 2802 | CG | MET | B | 139 | 18.504 | 72.111 | 102.319 | 1.00 | 43.14 | 6 |
| ATOM | 2803 | SD | MET | B | 139 | 19.493 | 72.042 | 100.831 | 1.00 | 40.08 | 16 |
| ATOM | 2804 | CE | MET | B | 139 | 20.758 | 70.861 | 101.301 | 1.00 | 38.50 | 6 |
| ATOM | 2805 | C | MET | B | 139 | 15.573 | 69.719 | 103.008 | 1.00 | 53.66 | 6 |
| ATOM | 2806 | O | MET | B | 139 | 15.803 | 68.589 | 103.416 | 1.00 | 53.85 | 8 |
| ATOM | 2807 | N | GLU | B | 140 | 14.538 | 70.027 | 102.242 | 1.00 | 57.95 | 7 |
| ATOM | 2808 | CA | GLU | B | 140 | 13.577 | 69.038 | 101.801 | 1.00 | 57.39 | 6 |
| ATOM | 2809 | CB | GLU | B | 140 | 12.251 | 69.169 | 102.554 | 1.00 | 79.51 | 6 |
| ATOM | 2810 | CG | GLU | B | 140 | 12.344 | 69.115 | 104.062 | 1.00 | 80.34 | 6 |
| ATOM | 2811 | CD | GLU | B | 140 | 10.978 | 69.016 | 104.705 | 1.00 | 81.16 | 6 |
| ATOM | 2812 | OE1 | GLU | B | 140 | 10.080 | 69.809 | 104.340 | 1.00 | 81.49 | 8 |
| ATOM | 2813 | OE2 | GLU | B | 140 | 10.800 | 68.144 | 105.578 | 1.00 | 80.96 | 8 |
| ATOM | 2814 | C | GLU | B | 140 | 13.351 | 69.381 | 100.345 | 1.00 | 57.83 | 6 |
| ATOM | 2815 | O | GLU | B | 140 | 12.263 | 69.808 | 99.966 | 1.00 | 58.17 | 8 |
| ATOM | 2816 | N | VAL | B | 141 | 14.389 | 69.218 | 99.535 | 1.00 | 69.25 | 7 |
| ATOM | 2817 | CA | VAL | B | 141 | 14.301 | 69.528 | 98.111 | 1.00 | 68.04 | 6 |
| ATOM | 2818 | CB | VAL | B | 141 | 15.729 | 69.592 | 97.478 | 1.00 | 52.16 | 6 |
| ATOM | 2819 | CG1 | VAL | B | 141 | 16.380 | 68.213 | 97.492 | 1.00 | 52.26 | 6 |
| ATOM | 2820 | CG2 | VAL | B | 141 | 15.657 | 70.129 | 96.069 | 1.00 | 52.61 | 6 |
| ATOM | 2821 | C | VAL | B | 141 | 13.435 | 68.510 | 97.352 | 1.00 | 68.81 | 6 |
| ATOM | 2822 | O | VAL | B | 141 | 13.279 | 67.368 | 97.781 | 1.00 | 68.33 | 8 |
| ATOM | 2823 | N | ARG | B | 142 | 12.849 | 68.947 | 96.242 | 1.00 | 88.90 | 7 |
| ATOM | 2824 | CA | ARG | B | 142 | 12.028 | 68.076 | 95.412 | 1.00 | 89.77 | 6 |
| ATOM | 2825 | CB | ARG | B | 142 | 10.783 | 68.817 | 94.911 | 1.00 | 81.59 | 6 |
| ATOM | 2826 | CG | ARG | B | 142 | 10.014 | 68.064 | 93.832 | 1.00 | 85.37 | 6 |
| ATOM | 2827 | CD | ARG | B | 142 | 8.820 | 68.839 | 93.298 | 1.00 | 88.90 | 6 |
| ATOM | 2828 | NE | ARG | B | 142 | 8.096 | 68.065 | 92.287 | 1.00 | 92.35 | 7 |
| ATOM | 2829 | CZ | ARG | B | 142 | 6.875 | 68.355 | 91.835 | 1.00 | 93.50 | 6 |
| ATOM | 2830 | NH1 | ARG | B | 142 | 6.214 | 69.409 | 92.298 | 1.00 | 93.60 | 7 |
| ATOM | 2831 | NH2 | ARG | B | 142 | 6.312 | 67.588 | 90.910 | 1.00 | 94.27 | 7 |
| ATOM | 2832 | C | ARG | B | 142 | 12.882 | 67.669 | 94.219 | 1.00 | 90.42 | 6 |
| ATOM | 2833 | O | ARG | B | 142 | 13.995 | 68.165 | 94.050 | 1.00 | 91.24 | 8 |
| ATOM | 2834 | N | VAL | B | 143 | 12.369 | 66.752 | 93.406 | 1.00 | 89.15 | 7 |
| ATOM | 2835 | CA | VAL | B | 143 | 13.062 | 66.298 | 92.206 | 1.00 | 87.96 | 6 |
| ATOM | 2836 | CB | VAL | B | 143 | 13.926 | 65.042 | 92.491 | 1.00 | 37.59 | 6 |
| ATOM | 2837 | CG1 | VAL | B | 143 | 14.830 | 64.755 | 91.306 | 1.00 | 37.23 | 6 |
| ATOM | 2838 | CG2 | VAL | B | 143 | 14.788 | 65.269 | 93.739 | 1.00 | 36.71 | 6 |
| ATOM | 2839 | C | VAL | B | 143 | 11.955 | 66.020 | 91.182 | 1.00 | 89.07 | 6 |
| ATOM | 2840 | O | VAL | B | 143 | 10.777 | 66.012 | 91.537 | 1.00 | 89.12 | 8 |
| ATOM | 2841 | N | ASP | B | 144 | 12.310 | 65.795 | 89.922 | 1.00 | 83.46 | 7 |
| ATOM | 2842 | CA | ASP | B | 144 | 11.277 | 65.607 | 88.905 | 1.00 | 83.97 | 6 |
| ATOM | 2843 | CB | ASP | B | 144 | 10.793 | 66.988 | 88.454 | 1.00 | 69.07 | 6 |
| ATOM | 2844 | CG | ASP | B | 144 | 9.328 | 67.011 | 88.100 | 1.00 | 70.86 | 6 |
| ATOM | 2845 | OD1 | ASP | B | 144 | 8.506 | 67.270 | 89.006 | 1.00 | 71.25 | 8 |
| ATOM | 2846 | OD2 | ASP | B | 144 | 8.998 | 66.765 | 86.918 | 1.00 | 71.19 | 8 |
| ATOM | 2847 | C | ASP | B | 144 | 11.687 | 64.807 | 87.665 | 1.00 | 85.08 | 6 |
| ATOM | 2848 | O | ASP | B | 144 | 12.858 | 64.501 | 87.448 | 1.00 | 85.87 | 8 |
| ATOM | 2849 | N | ARG | B | 145 | 10.694 | 64.491 | 86.843 | 1.00 | 81.25 | 7 |
| ATOM | 2850 | CA | ARG | B | 145 | 10.897 | 63.752 | 85.606 | 1.00 | 81.48 | 6 |
| ATOM | 2851 | CB | ARG | B | 145 | 9.883 | 62.598 | 85.526 | 1.00 | 116.67 | 6 |
| ATOM | 2852 | CG | ARG | B | 145 | 10.197 | 61.495 | 84.513 | 1.00 | 121.00 | 6 |
| ATOM | 2853 | CD | ARG | B | 145 | 9.541 | 61.742 | 83.154 | 1.00 | 124.57 | 6 |
| ATOM | 2854 | NE | ARG | B | 145 | 9.884 | 60.704 | 82.178 | 1.00 | 127.18 | 7 |
| ATOM | 2855 | CZ | ARG | B | 145 | 9.427 | 60.664 | 80.929 | 1.00 | 128.14 | 6 |
| ATOM | 2856 | NH1 | ARG | B | 145 | 8.598 | 61.604 | 80.485 | 1.00 | 127.70 | 7 |
| ATOM | 2857 | NH2 | ARG | B | 145 | 9.800 | 59.682 | 80.119 | 1.00 | 128.38 | 7 |
| ATOM | 2858 | C | ARG | B | 145 | 10.633 | 64.790 | 84.517 | 1.00 | 80.76 | 6 |
| ATOM | 2859 | O | ARG | B | 145 | 9.554 | 65.390 | 84.488 | 1.00 | 81.40 | 8 |
| ATOM | 2860 | N | GLY | B | 146 | 11.611 | 65.015 | 83.636 | 1.00 | 59.75 | 7 |
| ATOM | 2861 | CA | GLY | B | 146 | 11.434 | 66.010 | 82.586 | 1.00 | 58.12 | 6 |
| ATOM | 2862 | C | GLY | B | 146 | 11.790 | 65.634 | 81.154 | 1.00 | 57.76 | 6 |
| ATOM | 2863 | O | GLY | B | 146 | 12.133 | 64.484 | 80.855 | 1.00 | 57.87 | 8 |
| ATOM | 2864 | N | VAL | B | 147 | 11.711 | 66.630 | 80.271 | 1.00 | 55.35 | 7 |
| ATOM | 2865 | CA | VAL | B | 147 | 11.991 | 66.453 | 78.849 | 1.00 | 54.41 | 6 |
| ATOM | 2866 | CB | VAL | B | 147 | 10.865 | 67.038 | 77.992 | 1.00 | 80.10 | 6 |
| ATOM | 2867 | CG1 | VAL | B | 147 | 9.570 | 66.311 | 78.277 | 1.00 | 81.12 | 6 |
| ATOM | 2868 | CG2 | VAL | B | 147 | 10.715 | 68.523 | 78.281 | 1.00 | 81.16 | 6 |
| ATOM | 2869 | C | VAL | B | 147 | 13.293 | 67.083 | 78.369 | 1.00 | 53.79 | 6 |
| ATOM | 2870 | O | VAL | B | 147 | 14.112 | 66.411 | 77.744 | 1.00 | 54.45 | 8 |
| ATOM | 2871 | N | GLY | B | 148 | 13.477 | 68.373 | 78.635 | 1.00 | 56.94 | 7 |
| ATOM | 2872 | CA | GLY | B | 148 | 14.690 | 69.041 | 78.202 | 1.00 | 56.02 | 6 |
| ATOM | 2873 | C | GLY | B | 148 | 15.307 | 69.932 | 79.266 | 1.00 | 55.80 | 6 |
| ATOM | 2874 | O | GLY | B | 148 | 16.032 | 69.462 | 80.152 | 1.00 | 55.58 | 8 |
| ATOM | 2875 | N | TYR | B | 149 | 15.024 | 71.229 | 79.163 | 1.00 | 62.76 | 7 |
| ATOM | 2876 | CA | TYR | B | 149 | 15.519 | 72.225 | 80.109 | 1.00 | 61.67 | 6 |
| ATOM | 2877 | CB | TYR | B | 149 | 16.755 | 72.942 | 79.544 | 1.00 | 47.58 | 6 |
| ATOM | 2878 | CG | TYR | B | 149 | 17.298 | 74.030 | 80.448 | 1.00 | 44.27 | 6 |
| ATOM | 2879 | CD1 | TYR | B | 149 | 18.358 | 73.778 | 81.314 | 1.00 | 43.41 | 6 |
| ATOM | 2880 | CE1 | TYR | B | 149 | 18.818 | 74.761 | 82.198 | 1.00 | 42.32 | 6 |
| ATOM | 2881 | CD2 | TYR | B | 149 | 16.710 | 75.302 | 80.480 | 1.00 | 42.67 | 6 |
| ATOM | 2882 | CE2 | TYR | B | 149 | 17.159 | 76.292 | 81.360 | 1.00 | 41.68 | 6 |
| ATOM | 2883 | CZ | TYR | B | 149 | 18.212 | 76.010 | 82.219 | 1.00 | 42.28 | 6 |
| ATOM | 2884 | OH | TYR | B | 149 | 18.644 | 76.950 | 83.128 | 1.00 | 41.87 | 8 |
| ATOM | 2885 | C | TYR | B | 149 | 14.405 | 73.240 | 80.331 | 1.00 | 63.16 | 6 |
| ATOM | 2886 | O | TYR | B | 149 | 13.682 | 73.576 | 79.398 | 1.00 | 63.11 | 8 |
| ATOM | 2887 | N | VAL | B | 150 | 14.255 | 73.716 | 81.563 | 1.00 | 82.04 | 7 |
| ATOM | 2888 | CA | VAL | B | 150 | 13.236 | 74.716 | 81.863 | 1.00 | 83.70 | 6 |
| ATOM | 2889 | CB | VAL | B | 150 | 11.893 | 74.089 | 82.226 | 1.00 | 46.67 | 6 |
| ATOM | 2890 | CG1 | VAL | B | 150 | 10.820 | 74.625 | 81.293 | 1.00 | 46.16 | 6 |
| ATOM | 2891 | CG2 | VAL | B | 150 | 11.979 | 72.589 | 82.146 | 1.00 | 46.53 | 6 |
| ATOM | 2892 | C | VAL | B | 150 | 13.656 | 75.634 | 82.997 | 1.00 | 86.08 | 6 |
| ATOM | 2893 | O | VAL | B | 150 | 14.025 | 75.186 | 84.079 | 1.00 | 85.92 | 8 |
| ATOM | 2894 | N | PRO | B | 151 | 13.597 | 76.946 | 82.756 | 1.00 | 71.11 | 7 |
| ATOM | 2895 | CD | PRO | B | 151 | 13.161 | 77.521 | 81.472 | 1.00 | 58.08 | 6 |
| ATOM | 2896 | CA | PRO | B | 151 | 13.962 | 77.999 | 83.708 | 1.00 | 73.95 | 6 |
| ATOM | 2897 | CB | PRO | B | 151 | 13.869 | 79.267 | 82.854 | 1.00 | 60.58 | 6 |
| ATOM | 2898 | CG | PRO | B | 151 | 12.799 | 78.926 | 81.856 | 1.00 | 59.02 | 6 |
| ATOM | 2899 | C | PRO | B | 151 | 13.101 | 78.078 | 84.972 | 1.00 | 76.59 | 6 |
| ATOM | 2900 | O | PRO | B | 151 | 12.677 | 77.055 | 85.511 | 1.00 | 76.74 | 8 |
| ATOM | 2901 | N | ALA | B | 152 | 12.883 | 79.308 | 85.444 | 1.00 | 39.27 | 7 |
| ATOM | 2902 | CA | ALA | B | 152 | 12.057 | 79.598 | 86.616 | 1.00 | 42.47 | 6 |
| ATOM | 2903 | CB | ALA | B | 152 | 12.676 | 80.736 | 87.413 | 1.00 | 107.83 | 6 |
| ATOM | 2904 | C | ALA | B | 152 | 10.635 | 79.979 | 86.157 | 1.00 | 44.62 | 6 |
| ATOM | 2905 | O | ALA | B | 152 | 10.154 | 81.093 | 86.386 | 1.00 | 44.85 | 8 |
| ATOM | 2906 | N | GLU | B | 153 | 9.974 | 79.035 | 85.497 | 1.00 | 173.69 | 7 |
| ATOM | 2907 | CA | GLU | B | 153 | 8.624 | 79.258 | 85.006 | 1.00 | 177.64 | 6 |
| ATOM | 2908 | CB | GLU | B | 153 | 8.640 | 79.422 | 83.487 | 1.00 | 172.29 | 6 |
| ATOM | 2909 | CG | GLU | B | 153 | 7.300 | 79.820 | 82.895 | 1.00 | 175.51 | 6 |
| ATOM | 2910 | CD | GLU | B | 153 | 6.805 | 81.159 | 83.414 | 1.00 | 177.58 | 6 |
| ATOM | 2911 | OE1 | GLU | B | 153 | 7.539 | 82.160 | 83.272 | 1.00 | 179.18 | 8 |
| ATOM | 2912 | OE2 | GLU | B | 153 | 5.681 | 81.214 | 83.959 | 1.00 | 179.05 | 8 |
| ATOM | 2913 | C | GLU | B | 153 | 7.711 | 78.100 | 85.399 | 1.00 | 179.79 | 6 |
| ATOM | 2914 | O | GLU | B | 153 | 6.486 | 78.190 | 85.286 | 1.00 | 180.54 | 8 |
| ATOM | 2915 | N | ARG | B | 154 | 8.313 | 77.010 | 85.863 | 1.00 | 116.41 | 7 |
| ATOM | 2916 | CA | ARG | B | 154 | 7.545 | 75.852 | 86.283 | 1.00 | 117.36 | 6 |
| ATOM | 2917 | CB | ARG | B | 154 | 8.356 | 74.572 | 86.077 | 1.00 | 160.53 | 6 |
| ATOM | 2918 | CG | ARG | B | 154 | 8.513 | 74.185 | 84.614 | 1.00 | 164.64 | 6 |
| ATOM | 2919 | CD | ARG | B | 154 | 7.156 | 73.958 | 83.940 | 1.00 | 168.10 | 6 |
| ATOM | 2920 | NE | ARG | B | 154 | 7.294 | 73.684 | 82.509 | 1.00 | 170.66 | 7 |
| ATOM | 2921 | CZ | ARG | B | 154 | 6.284 | 73.387 | 81.698 | 1.00 | 171.03 | 6 |
| ATOM | 2922 | NH1 | ARG | B | 154 | 5.046 | 73.321 | 82.168 | 1.00 | 171.38 | 7 |
| ATOM | 2923 | NH2 | ARG | B | 154 | 6.513 | 73.154 | 80.412 | 1.00 | 171.09 | 7 |
| ATOM | 2924 | C | ARG | B | 154 | 7.129 | 75.992 | 87.738 | 1.00 | 116.81 | 6 |
| ATOM | 2925 | O | ARG | B | 154 | 6.332 | 75.204 | 88.240 | 1.00 | 117.13 | 8 |
| ATOM | 2926 | N | HIS | B | 155 | 7.678 | 76.997 | 88.413 | 1.00 | 55.66 | 7 |
| ATOM | 2927 | CA | HIS | B | 155 | 7.348 | 77.274 | 89.818 | 1.00 | 55.25 | 6 |
| ATOM | 2928 | CB | HIS | B | 155 | 5.966 | 77.938 | 89.894 | 1.00 | 208.87 | 6 |
| ATOM | 2929 | CG | HIS | B | 155 | 5.783 | 79.078 | 88.934 | 1.00 | 208.87 | 6 |
| ATOM | 2930 | CD2 | HIS | B | 155 | 6.633 | 79.645 | 88.044 | 1.00 | 208.87 | 6 |
| ATOM | 2931 | ND1 | HIS | B | 155 | 4.588 | 79.753 | 88.801 | 1.00 | 208.87 | 7 |
| ATOM | 2932 | CE1 | HIS | B | 155 | 4.708 | 80.682 | 87.870 | 1.00 | 208.87 | 6 |
| ATOM | 2933 | NE2 | HIS | B | 155 | 5.939 | 80.638 | 87.394 | 1.00 | 208.87 | 7 |
| ATOM | 2934 | C | HIS | B | 155 | 7.390 | 76.045 | 90.758 | 1.00 | 53.61 | 6 |
| ATOM | 2935 | O | HIS | B | 155 | 6.705 | 75.033 | 90.546 | 1.00 | 52.58 | 8 |
| ATOM | 2936 | N | GLY | B | 156 | 8.191 | 76.165 | 91.815 | 1.00 | 77.00 | 7 |
| ATOM | 2937 | CA | GLY | B | 156 | 8.347 | 75.084 | 92.773 | 1.00 | 75.17 | 6 |
| ATOM | 2938 | C | GLY | B | 156 | 7.224 | 74.924 | 93.780 | 1.00 | 74.46 | 6 |
| ATOM | 2939 | O | GLY | B | 156 | 7.414 | 75.133 | 94.983 | 1.00 | 73.94 | 8 |
| ATOM | 2940 | N | ILE | B | 157 | 6.056 | 74.540 | 93.273 | 1.00 | 110.69 | 7 |
| ATOM | 2941 | CA | ILE | B | 157 | 4.861 | 74.317 | 94.081 | 1.00 | 109.94 | 6 |
| ATOM | 2942 | CB | ILE | B | 157 | 3.890 | 73.375 | 93.328 | 1.00 | 96.64 | 6 |
| ATOM | 2943 | CG2 | ILE | B | 157 | 2.576 | 73.271 | 94.066 | 1.00 | 97.13 | 6 |
| ATOM | 2944 | CG1 | ILE | B | 157 | 3.673 | 73.884 | 91.899 | 1.00 | 96.85 | 6 |
| ATOM | 2945 | CD1 | ILE | B | 157 | 3.199 | 75.326 | 91.800 | 1.00 | 97.40 | 6 |
| ATOM | 2946 | C | ILE | B | 157 | 5.211 | 73.711 | 95.447 | 1.00 | 109.72 | 6 |
| ATOM | 2947 | O | ILE | B | 157 | 5.476 | 74.440 | 96.403 | 1.00 | 109.75 | 8 |
| ATOM | 2948 | N | LYS | B | 158 | 5.204 | 72.381 | 95.523 | 1.00 | 105.17 | 7 |
| ATOM | 2949 | CA | LYS | B | 158 | 5.531 | 71.639 | 96.742 | 1.00 | 104.52 | 6 |
| ATOM | 2950 | CB | LYS | B | 158 | 7.013 | 71.256 | 96.743 | 1.00 | 126.04 | 6 |
| ATOM | 2951 | CG | LYS | B | 158 | 7.335 | 70.011 | 95.942 | 1.00 | 127.28 | 6 |
| ATOM | 2952 | CD | LYS | B | 158 | 6.666 | 68.780 | 96.535 | 1.00 | 127.58 | 6 |
| ATOM | 2953 | CE | LYS | B | 158 | 7.045 | 67.522 | 95.767 | 1.00 | 128.34 | 6 |
| ATOM | 2954 | NZ | LYS | B | 158 | 6.394 | 66.299 | 96.313 | 1.00 | 128.78 | 7 |
| ATOM | 2955 | C | LYS | B | 158 | 5.211 | 72.330 | 98.060 | 1.00 | 104.50 | 6 |
| ATOM | 2956 | O | LYS | B | 158 | 4.048 | 72.604 | 98.361 | 1.00 | 104.97 | 8 |
| ATOM | 2957 | N | ASP | B | 159 | 6.265 | 72.587 | 98.839 | 1.00 | 70.07 | 7 |
| ATOM | 2958 | CA | ASP | B | 159 | 6.186 | 73.231 | 100.155 | 1.00 | 68.58 | 6 |
| ATOM | 2959 | CB | ASP | B | 159 | 7.601 | 73.320 | 100.749 | 1.00 | 75.47 | 6 |
| ATOM | 2960 | CG | ASP | B | 159 | 7.617 | 73.852 | 102.174 | 1.00 | 76.09 | 6 |
| ATOM | 2961 | OD1 | ASP | B | 159 | 6.953 | 73.253 | 103.054 | 1.00 | 76.19 | 8 |
| ATOM | 2962 | OD2 | ASP | B | 159 | 8.307 | 74.871 | 102.412 | 1.00 | 76.49 | 8 |
| ATOM | 2963 | C | ASP | B | 159 | 5.546 | 74.624 | 100.102 | 1.00 | 68.26 | 6 |
| ATOM | 2964 | O | ASP | B | 159 | 6.104 | 75.593 | 100.612 | 1.00 | 67.90 | 8 |
| ATOM | 2965 | N | ARG | B | 160 | 4.365 | 74.697 | 99.492 | 1.00 | 91.05 | 7 |
| ATOM | 2966 | CA | ARG | B | 160 | 3.605 | 75.932 | 99.333 | 1.00 | 91.10 | 6 |
| ATOM | 2967 | CB | ARG | B | 160 | 2.606 | 76.097 | 100.489 | 1.00 | 196.98 | 6 |
| ATOM | 2968 | CG | ARG | B | 160 | 1.425 | 77.020 | 100.169 | 1.00 | 199.78 | 6 |
| ATOM | 2969 | CD | ARG | B | 160 | 0.318 | 76.898 | 101.215 | 1.00 | 201.81 | 6 |
| ATOM | 2970 | NE | ARG | B | 160 | −0.931 | 77.522 | 100.779 | 1.00 | 202.57 | 7 |
| ATOM | 2971 | CZ | ARG | B | 160 | −2.067 | 77.484 | 101.471 | 1.00 | 202.64 | 6 |
| ATOM | 2972 | NH1 | ARG | B | 160 | −2.119 | 76.853 | 102.637 | 1.00 | 203.08 | 7 |
| ATOM | 2973 | NH2 | ARG | B | 160 | −3.156 | 78.074 | 100.997 | 1.00 | 202.29 | 7 |
| ATOM | 2974 | C | ARG | B | 160 | 4.508 | 77.156 | 99.235 | 1.00 | 90.17 | 6 |
| ATOM | 2975 | O | ARG | B | 160 | 4.862 | 77.576 | 98.131 | 1.00 | 90.77 | 8 |
| ATOM | 2976 | N | ILE | B | 161 | 4.881 | 77.714 | 100.388 | 1.00 | 111.41 | 7 |
| ATOM | 2977 | CA | ILE | B | 161 | 5.742 | 78.897 | 100.453 | 1.00 | 108.90 | 6 |
| ATOM | 2978 | CB | ILE | B | 161 | 5.186 | 79.910 | 101.467 | 1.00 | 155.30 | 6 |
| ATOM | 2979 | CG2 | ILE | B | 161 | 5.920 | 81.240 | 101.329 | 1.00 | 155.26 | 6 |
| ATOM | 2980 | CG1 | ILE | B | 161 | 3.689 | 80.101 | 101.221 | 1.00 | 156.01 | 6 |
| ATOM | 2981 | CD1 | ILE | B | 161 | 2.979 | 80.897 | 102.286 | 1.00 | 157.04 | 6 |
| ATOM | 2982 | C | ILE | B | 161 | 7.184 | 78.537 | 100.831 | 1.00 | 106.94 | 6 |
| ATOM | 2983 | O | ILE | B | 161 | 7.423 | 77.547 | 101.521 | 1.00 | 106.37 | 8 |
| ATOM | 2984 | N | ASN | B | 162 | 8.131 | 79.360 | 100.381 | 1.00 | 157.69 | 7 |
| ATOM | 2985 | CA | ASN | B | 162 | 9.562 | 79.144 | 100.613 | 1.00 | 155.50 | 6 |
| ATOM | 2986 | CB | ASN | B | 162 | 9.848 | 78.754 | 102.065 | 1.00 | 120.56 | 6 |
| ATOM | 2987 | CG | ASN | B | 162 | 9.259 | 79.725 | 103.054 | 1.00 | 120.85 | 6 |
| ATOM | 2988 | OD1 | ASN | B | 162 | 9.299 | 80.936 | 102.847 | 1.00 | 120.63 | 8 |
| ATOM | 2989 | ND2 | ASN | B | 162 | 8.715 | 79.201 | 104.148 | 1.00 | 121.34 | 7 |
| ATOM | 2990 | C | ASN | B | 162 | 9.993 | 78.014 | 99.692 | 1.00 | 154.09 | 6 |
| ATOM | 2991 | O | ASN | B | 162 | 10.002 | 76.847 | 100.086 | 1.00 | 154.31 | 8 |
| ATOM | 2992 | N | ALA | B | 163 | 10.354 | 78.364 | 98.463 | 1.00 | 77.76 | 7 |
| ATOM | 2993 | CA | ALA | B | 163 | 10.735 | 77.354 | 97.496 | 1.00 | 74.56 | 6 |
| ATOM | 2994 | CB | ALA | B | 163 | 9.571 | 77.121 | 96.541 | 1.00 | 55.98 | 6 |
| ATOM | 2995 | C | ALA | B | 163 | 12.007 | 77.639 | 96.703 | 1.00 | 72.69 | 6 |
| ATOM | 2996 | O | ALA | B | 163 | 12.746 | 76.715 | 96.355 | 1.00 | 73.43 | 8 |
| ATOM | 2997 | N | ILE | B | 164 | 12.274 | 78.904 | 96.410 | 1.00 | 59.09 | 7 |
| ATOM | 2998 | CA | ILE | B | 164 | 13.459 | 79.242 | 95.626 | 1.00 | 57.08 | 6 |
| ATOM | 2999 | CB | ILE | B | 164 | 14.715 | 79.360 | 96.542 | 1.00 | 43.29 | 6 |
| ATOM | 3000 | CG2 | ILE | B | 164 | 14.450 | 80.364 | 97.641 | 1.00 | 43.45 | 6 |
| ATOM | 3001 | CG1 | ILE | B | 164 | 15.046 | 78.026 | 97.206 | 1.00 | 42.16 | 6 |
| ATOM | 3002 | CD1 | ILE | B | 164 | 16.244 | 78.112 | 98.133 | 1.00 | 41.64 | 6 |
| ATOM | 3003 | C | ILE | B | 164 | 13.681 | 78.188 | 94.520 | 1.00 | 55.27 | 6 |
| ATOM | 3004 | O | ILE | B | 164 | 14.807 | 77.762 | 94.253 | 1.00 | 55.18 | 8 |
| ATOM | 3005 | N | PRO | B | 165 | 12.590 | 77.792 | 93.835 | 1.00 | 54.05 | 7 |
| ATOM | 3006 | CD | PRO | B | 165 | 11.340 | 78.566 | 93.734 | 1.00 | 45.24 | 6 |
| ATOM | 3007 | CA | PRO | B | 165 | 12.634 | 76.794 | 92.768 | 1.00 | 53.19 | 6 |
| ATOM | 3008 | CB | PRO | B | 165 | 11.277 | 76.955 | 92.083 | 1.00 | 44.74 | 6 |
| ATOM | 3009 | CG | PRO | B | 165 | 10.973 | 78.380 | 92.281 | 1.00 | 44.95 | 6 |
| ATOM | 3010 | C | PRO | B | 165 | 13.781 | 76.948 | 91.798 | 1.00 | 51.81 | 6 |
| ATOM | 3011 | O | PRO | B | 165 | 13.890 | 77.945 | 91.093 | 1.00 | 51.05 | 8 |
| ATOM | 3012 | N | VAL | B | 166 | 14.638 | 75.941 | 91.773 | 1.00 | 53.76 | 7 |
| ATOM | 3013 | CA | VAL | B | 166 | 15.772 | 75.942 | 90.877 | 1.00 | 52.63 | 6 |
| ATOM | 3014 | CB | VAL | B | 166 | 16.795 | 74.880 | 91.303 | 1.00 | 37.36 | 6 |
| ATOM | 3015 | CG1 | VAL | B | 166 | 17.762 | 75.491 | 92.294 | 1.00 | 37.27 | 6 |
| ATOM | 3016 | CG2 | VAL | B | 166 | 16.088 | 73.698 | 91.955 | 1.00 | 37.18 | 6 |
| ATOM | 3017 | C | VAL | B | 166 | 15.295 | 75.691 | 89.452 | 1.00 | 53.14 | 6 |
| ATOM | 3018 | O | VAL | B | 166 | 14.093 | 75.552 | 89.214 | 1.00 | 52.50 | 8 |
| ATOM | 3019 | N | ASP | B | 167 | 16.228 | 75.648 | 88.507 | 1.00 | 51.01 | 7 |
| ATOM | 3020 | CA | ASP | B | 167 | 15.860 | 75.429 | 87.116 | 1.00 | 51.58 | 6 |
| ATOM | 3021 | CB | ASP | B | 167 | 16.843 | 76.149 | 86.193 | 1.00 | 56.28 | 6 |
| ATOM | 3022 | CG | ASP | B | 167 | 16.933 | 77.631 | 86.490 | 1.00 | 56.37 | 6 |
| ATOM | 3023 | OD1 | ASP | B | 167 | 15.857 | 78.266 | 86.604 | 1.00 | 56.07 | 8 |
| ATOM | 3024 | OD2 | ASP | B | 167 | 18.067 | 78.157 | 86.608 | 1.00 | 55.57 | 8 |
| ATOM | 3025 | C | ASP | B | 167 | 15.785 | 73.942 | 86.771 | 1.00 | 52.93 | 6 |
| ATOM | 3026 | O | ASP | B | 167 | 16.523 | 73.117 | 87.316 | 1.00 | 54.06 | 8 |
| ATOM | 3027 | N | ALA | B | 168 | 14.880 | 73.612 | 85.860 | 1.00 | 70.74 | 7 |
| ATOM | 3028 | CA | ALA | B | 168 | 14.660 | 72.237 | 85.446 | 1.00 | 72.04 | 6 |
| ATOM | 3029 | CB | ALA | B | 168 | 13.238 | 72.089 | 84.963 | 1.00 | 93.07 | 6 |
| ATOM | 3030 | C | ALA | B | 168 | 15.621 | 71.764 | 84.368 | 1.00 | 73.25 | 6 |
| ATOM | 3031 | O | ALA | B | 168 | 15.318 | 71.848 | 83.176 | 1.00 | 74.88 | 8 |
| ATOM | 3032 | N | ILE | B | 169 | 16.774 | 71.254 | 84.784 | 1.00 | 40.64 | 7 |
| ATOM | 3033 | CA | ILE | B | 169 | 17.757 | 70.769 | 83.832 | 1.00 | 41.50 | 6 |
| ATOM | 3034 | CB | ILE | B | 169 | 19.213 | 71.068 | 84.313 | 1.00 | 98.30 | 6 |
| ATOM | 3035 | CG2 | ILE | B | 169 | 19.539 | 72.525 | 84.130 | 1.00 | 97.49 | 6 |
| ATOM | 3036 | CG1 | ILE | B | 169 | 19.381 | 70.705 | 85.793 | 1.00 | 100.66 | 6 |
| ATOM | 3037 | CD1 | ILE | B | 169 | 19.738 | 69.249 | 86.070 | 1.00 | 102.42 | 6 |
| ATOM | 3038 | C | ILE | B | 169 | 17.619 | 69.273 | 83.586 | 1.00 | 41.74 | 6 |
| ATOM | 3039 | O | ILE | B | 169 | 18.592 | 68.532 | 83.729 | 1.00 | 42.04 | 8 |
| ATOM | 3040 | N | PHE | B | 170 | 16.432 | 68.802 | 83.211 | 1.00 | 42.29 | 7 |
| ATOM | 3041 | CA | PHE | B | 170 | 16.317 | 67.361 | 82.977 | 1.00 | 43.20 | 6 |
| ATOM | 3042 | CB | PHE | B | 170 | 15.013 | 66.777 | 83.538 | 1.00 | 61.40 | 6 |
| ATOM | 3043 | CG | PHE | B | 170 | 13.954 | 67.786 | 83.836 | 1.00 | 63.03 | 6 |
| ATOM | 3044 | CD1 | PHE | B | 170 | 13.399 | 68.556 | 82.827 | 1.00 | 63.24 | 6 |
| ATOM | 3045 | CD2 | PHE | B | 170 | 13.438 | 67.889 | 85.124 | 1.00 | 63.04 | 6 |
| ATOM | 3046 | CE1 | PHE | B | 170 | 12.338 | 69.403 | 83.094 | 1.00 | 62.61 | 6 |
| ATOM | 3047 | CE2 | PHE | B | 170 | 12.381 | 68.732 | 85.398 | 1.00 | 62.50 | 6 |
| ATOM | 3048 | CZ | PHE | B | 170 | 11.827 | 69.489 | 84.379 | 1.00 | 62.13 | 6 |
| ATOM | 3049 | C | PHE | B | 170 | 16.502 | 66.878 | 81.545 | 1.00 | 42.83 | 6 |
| ATOM | 3050 | O | PHE | B | 170 | 15.619 | 67.031 | 80.700 | 1.00 | 42.08 | 8 |
| ATOM | 3051 | N | SER | B | 171 | 17.672 | 66.276 | 81.318 | 1.00 | 58.08 | 7 |
| ATOM | 3052 | CA | SER | B | 171 | 18.105 | 65.721 | 80.035 | 1.00 | 57.84 | 6 |
| ATOM | 3053 | CB | SER | B | 171 | 18.167 | 66.810 | 78.959 | 1.00 | 64.81 | 6 |
| ATOM | 3054 | OG | SER | B | 171 | 16.915 | 67.437 | 78.764 | 1.00 | 66.33 | 8 |
| ATOM | 3055 | C | SER | B | 171 | 19.519 | 65.173 | 80.243 | 1.00 | 58.17 | 6 |
| ATOM | 3056 | O | SER | B | 171 | 20.482 | 65.929 | 80.186 | 1.00 | 58.94 | 8 |
| ATOM | 3057 | N | PRO | B | 172 | 19.663 | 63.858 | 80.480 | 1.00 | 62.96 | 7 |
| ATOM | 3058 | CD | PRO | B | 172 | 18.555 | 62.888 | 80.478 | 1.00 | 125.39 | 6 |
| ATOM | 3059 | CA | PRO | B | 172 | 20.948 | 63.178 | 80.702 | 1.00 | 62.05 | 6 |
| ATOM | 3060 | CB | PRO | B | 172 | 20.586 | 61.714 | 80.509 | 1.00 | 124.61 | 6 |
| ATOM | 3061 | CG | PRO | B | 172 | 19.206 | 61.658 | 81.068 | 1.00 | 125.56 | 6 |
| ATOM | 3062 | C | PRO | B | 172 | 22.105 | 63.621 | 79.791 | 1.00 | 62.13 | 6 |
| ATOM | 3063 | O | PRO | B | 172 | 23.274 | 63.464 | 80.141 | 1.00 | 62.81 | 8 |
| ATOM | 3064 | N | VAL | B | 173 | 21.774 | 64.151 | 78.617 | 1.00 | 50.25 | 7 |
| ATOM | 3065 | CA | VAL | B | 173 | 22.775 | 64.643 | 77.679 | 1.00 | 49.25 | 6 |
| ATOM | 3066 | CB | VAL | B | 173 | 22.246 | 64.633 | 76.232 | 1.00 | 47.86 | 6 |
| ATOM | 3067 | CG1 | VAL | B | 173 | 21.865 | 63.242 | 75.819 | 1.00 | 46.05 | 6 |
| ATOM | 3068 | CG2 | VAL | B | 173 | 21.033 | 65.531 | 76.123 | 1.00 | 48.20 | 6 |
| ATOM | 3069 | C | VAL | B | 173 | 22.992 | 66.093 | 78.087 | 1.00 | 50.28 | 6 |
| ATOM | 3070 | O | VAL | B | 173 | 22.022 | 66.787 | 78.407 | 1.00 | 50.57 | 8 |
| ATOM | 3071 | N | ARG | B | 174 | 24.238 | 66.565 | 78.080 | 1.00 | 46.53 | 7 |
| ATOM | 3072 | CA | ARG | B | 174 | 24.500 | 67.957 | 78.459 | 1.00 | 45.95 | 6 |
| ATOM | 3073 | CB | ARG | B | 174 | 25.016 | 68.000 | 79.903 | 1.00 | 76.44 | 6 |
| ATOM | 3074 | CG | ARG | B | 174 | 23.959 | 67.598 | 80.932 | 1.00 | 76.70 | 6 |
| ATOM | 3075 | CD | ARG | B | 174 | 24.453 | 67.729 | 82.365 | 1.00 | 77.00 | 6 |
| ATOM | 3076 | NE | ARG | B | 174 | 23.374 | 67.503 | 83.323 | 1.00 | 77.86 | 7 |
| ATOM | 3077 | CZ | ARG | B | 174 | 23.543 | 67.416 | 84.639 | 1.00 | 78.64 | 6 |
| ATOM | 3078 | NH1 | ARG | B | 174 | 24.754 | 67.535 | 85.169 | 1.00 | 79.52 | 7 |
| ATOM | 3079 | NH2 | ARG | B | 174 | 22.497 | 67.208 | 85.429 | 1.00 | 78.53 | 7 |
| ATOM | 3080 | C | ARG | B | 174 | 25.447 | 68.725 | 77.531 | 1.00 | 45.02 | 6 |
| ATOM | 3081 | O | ARG | B | 174 | 26.133 | 69.643 | 77.966 | 1.00 | 45.18 | 8 |
| ATOM | 3082 | N | ARG | B | 175 | 25.449 | 68.359 | 76.251 | 1.00 | 30.81 | 7 |
| ATOM | 3083 | CA | ARG | B | 175 | 26.307 | 68.974 | 75.232 | 1.00 | 30.24 | 6 |
| ATOM | 3084 | CB | ARG | B | 175 | 27.705 | 69.252 | 75.796 | 1.00 | 56.73 | 6 |
| ATOM | 3085 | CG | ARG | B | 175 | 28.609 | 69.963 | 74.814 | 1.00 | 58.11 | 6 |
| ATOM | 3086 | CD | ARG | B | 175 | 30.066 | 69.866 | 75.184 | 1.00 | 58.93 | 6 |
| ATOM | 3087 | NE | ARG | B | 175 | 30.879 | 70.341 | 74.074 | 1.00 | 60.67 | 7 |
| ATOM | 3088 | CZ | ARG | B | 175 | 32.160 | 70.044 | 73.914 | 1.00 | 61.35 | 6 |
| ATOM | 3089 | NH1 | ARG | B | 175 | 32.771 | 69.274 | 74.800 | 1.00 | 62.34 | 7 |
| ATOM | 3090 | NH2 | ARG | B | 175 | 32.821 | 70.495 | 72.859 | 1.00 | 61.62 | 7 |
| ATOM | 3091 | C | ARG | B | 175 | 26.434 | 68.025 | 74.030 | 1.00 | 29.61 | 6 |
| ATOM | 3092 | O | ARG | B | 175 | 26.804 | 66.867 | 74.174 | 1.00 | 28.19 | 8 |
| ATOM | 3093 | N | VAL | B | 176 | 26.163 | 68.521 | 72.837 | 1.00 | 43.00 | 7 |
| ATOM | 3094 | CA | VAL | B | 176 | 26.209 | 67.663 | 71.674 | 1.00 | 42.87 | 6 |
| ATOM | 3095 | CB | VAL | B | 176 | 24.779 | 67.226 | 71.310 | 1.00 | 59.69 | 6 |
| ATOM | 3096 | CG1 | VAL | B | 176 | 24.808 | 66.209 | 70.197 | 1.00 | 61.48 | 6 |
| ATOM | 3097 | CG2 | VAL | B | 176 | 24.080 | 66.679 | 72.535 | 1.00 | 59.65 | 6 |
| ATOM | 3098 | C | VAL | B | 176 | 26.838 | 68.321 | 70.450 | 1.00 | 43.44 | 6 |
| ATOM | 3099 | O | VAL | B | 176 | 26.225 | 69.206 | 69.832 | 1.00 | 43.32 | 8 |
| ATOM | 3100 | N | ALA | B | 177 | 28.047 | 67.883 | 70.093 | 1.00 | 43.93 | 7 |
| ATOM | 3101 | CA | ALA | B | 177 | 28.750 | 68.414 | 68.917 | 1.00 | 44.13 | 6 |
| ATOM | 3102 | CB | ALA | B | 177 | 30.096 | 69.001 | 69.326 | 1.00 | 84.37 | 6 |
| ATOM | 3103 | C | ALA | B | 177 | 28.946 | 67.333 | 67.846 | 1.00 | 43.58 | 6 |
| ATOM | 3104 | O | ALA | B | 177 | 28.846 | 66.139 | 68.127 | 1.00 | 43.19 | 8 |
| ATOM | 3105 | N | PHE | B | 178 | 29.236 | 67.754 | 66.618 | 1.00 | 16.23 | 7 |
| ATOM | 3106 | CA | PHE | B | 178 | 29.413 | 66.806 | 65.523 | 1.00 | 17.34 | 6 |
| ATOM | 3107 | CB | PHE | B | 178 | 28.079 | 66.114 | 65.248 | 1.00 | 28.38 | 6 |
| ATOM | 3108 | CG | PHE | B | 178 | 27.105 | 66.970 | 64.492 | 1.00 | 26.31 | 6 |
| ATOM | 3109 | CD1 | PHE | B | 178 | 27.007 | 66.878 | 63.110 | 1.00 | 25.18 | 6 |
| ATOM | 3110 | CD2 | PHE | B | 178 | 26.323 | 67.910 | 65.151 | 1.00 | 24.98 | 6 |
| ATOM | 3111 | CE1 | PHE | B | 178 | 26.145 | 67.713 | 62.393 | 1.00 | 23.37 | 6 |
| ATOM | 3112 | CE2 | PHE | B | 178 | 25.458 | 68.754 | 64.434 | 1.00 | 23.56 | 6 |
| ATOM | 3113 | CZ | PHE | B | 178 | 25.370 | 68.653 | 63.057 | 1.00 | 22.91 | 6 |
| ATOM | 3114 | C | PHE | B | 178 | 29.912 | 67.458 | 64.227 | 1.00 | 18.91 | 6 |
| ATOM | 3115 | O | PHE | B | 178 | 29.345 | 68.451 | 63.769 | 1.00 | 17.56 | 8 |
| ATOM | 3116 | N | GLN | B | 179 | 30.963 | 66.908 | 63.625 | 1.00 | 62.17 | 7 |
| ATOM | 3117 | CA | GLN | B | 179 | 31.449 | 67.473 | 62.369 | 1.00 | 65.93 | 6 |
| ATOM | 3118 | CB | GLN | B | 179 | 32.773 | 68.234 | 62.560 | 1.00 | 80.10 | 6 |
| ATOM | 3119 | CG | GLN | B | 179 | 33.982 | 67.411 | 62.962 | 1.00 | 81.43 | 6 |
| ATOM | 3120 | CD | GLN | B | 179 | 35.287 | 68.139 | 62.658 | 1.00 | 82.38 | 6 |
| ATOM | 3121 | OE1 | GLN | B | 179 | 35.333 | 69.371 | 62.650 | 1.00 | 82.70 | 8 |
| ATOM | 3122 | NE2 | GLN | B | 179 | 36.355 | 67.379 | 62.417 | 1.00 | 81.97 | 7 |
| ATOM | 3123 | C | GLN | B | 179 | 31.606 | 66.428 | 61.282 | 1.00 | 66.88 | 6 |
| ATOM | 3124 | O | GLN | B | 179 | 32.281 | 65.425 | 61.468 | 1.00 | 67.65 | 8 |
| ATOM | 3125 | N | VAL | B | 180 | 30.979 | 66.674 | 60.140 | 1.00 | 30.90 | 7 |
| ATOM | 3126 | CA | VAL | B | 180 | 31.039 | 65.738 | 59.032 | 1.00 | 33.56 | 6 |
| ATOM | 3127 | CB | VAL | B | 180 | 29.649 | 65.640 | 58.339 | 1.00 | 68.73 | 6 |
| ATOM | 3128 | CG1 | VAL | B | 180 | 29.190 | 67.013 | 57.885 | 1.00 | 69.38 | 6 |
| ATOM | 3129 | CG2 | VAL | B | 180 | 29.706 | 64.663 | 57.172 | 1.00 | 69.19 | 6 |
| ATOM | 3130 | C | VAL | B | 180 | 32.127 | 66.030 | 57.990 | 1.00 | 35.36 | 6 |
| ATOM | 3131 | O | VAL | B | 180 | 31.912 | 66.779 | 57.033 | 1.00 | 35.29 | 8 |
| ATOM | 3132 | N | GLU | B | 181 | 33.296 | 65.428 | 58.168 | 1.00 | 62.66 | 7 |
| ATOM | 3133 | CA | GLU | B | 181 | 34.383 | 65.627 | 57.219 | 1.00 | 65.99 | 6 |
| ATOM | 3134 | CB | GLU | B | 181 | 35.724 | 65.329 | 57.896 | 1.00 | 120.88 | 6 |
| ATOM | 3135 | CG | GLU | B | 181 | 36.953 | 65.566 | 57.025 | 1.00 | 123.47 | 6 |
| ATOM | 3136 | CD | GLU | B | 181 | 38.245 | 65.511 | 57.823 | 1.00 | 124.36 | 6 |
| ATOM | 3137 | OE1 | GLU | B | 181 | 38.399 | 64.565 | 58.628 | 1.00 | 123.76 | 8 |
| ATOM | 3138 | OE2 | GLU | B | 181 | 39.108 | 66.402 | 57.644 | 1.00 | 125.31 | 8 |
| ATOM | 3139 | C | GLU | B | 181 | 34.175 | 64.714 | 56.004 | 1.00 | 67.27 | 6 |
| ATOM | 3140 | O | GLU | B | 181 | 33.316 | 63.842 | 56.019 | 1.00 | 68.13 | 8 |
| ATOM | 3141 | N | ASP | B | 182 | 34.943 | 64.921 | 54.945 | 1.00 | 43.73 | 7 |
| ATOM | 3142 | CA | ASP | B | 182 | 34.808 | 64.091 | 53.763 | 1.00 | 45.68 | 6 |
| ATOM | 3143 | CB | ASP | B | 182 | 35.001 | 64.914 | 52.486 | 1.00 | 97.05 | 6 |
| ATOM | 3144 | CG | ASP | B | 182 | 34.684 | 66.383 | 52.677 | 1.00 | 98.74 | 6 |
| ATOM | 3145 | OD1 | ASP | B | 182 | 33.808 | 66.704 | 53.506 | 1.00 | 100.74 | 8 |
| ATOM | 3146 | OD2 | ASP | B | 182 | 35.302 | 67.218 | 51.983 | 1.00 | 97.37 | 8 |
| ATOM | 3147 | C | ASP | B | 182 | 35.875 | 63.008 | 53.816 | 1.00 | 47.05 | 6 |
| ATOM | 3148 | O | ASP | B | 182 | 36.967 | 63.237 | 54.333 | 1.00 | 47.61 | 8 |
| ATOM | 3149 | N | THR | B | 183 | 35.548 | 61.832 | 53.285 | 1.00 | 51.94 | 7 |
| ATOM | 3150 | CA | THR | B | 183 | 36.461 | 60.686 | 53.225 | 1.00 | 53.53 | 6 |
| ATOM | 3151 | CB | THR | B | 183 | 36.225 | 59.684 | 54.366 | 1.00 | 71.65 | 6 |
| ATOM | 3152 | OG1 | THR | B | 183 | 34.860 | 59.248 | 54.340 | 1.00 | 71.78 | 8 |
| ATOM | 3153 | CG2 | THR | B | 183 | 36.547 | 60.318 | 55.704 | 1.00 | 72.82 | 6 |
| ATOM | 3154 | C | THR | B | 183 | 36.140 | 59.993 | 51.922 | 1.00 | 54.41 | 6 |
| ATOM | 3155 | O | THR | B | 183 | 34.991 | 60.012 | 51.479 | 1.00 | 54.35 | 8 |
| ATOM | 3156 | N | ALA | B | 184 | 37.137 | 59.360 | 51.319 | 1.00 | 129.81 | 7 |
| ATOM | 3157 | CA | ALA | B | 184 | 36.918 | 58.709 | 50.042 | 1.00 | 131.82 | 6 |
| ATOM | 3158 | CB | ALA | B | 184 | 37.776 | 59.367 | 48.990 | 1.00 | 58.25 | 6 |
| ATOM | 3159 | C | ALA | B | 184 | 37.140 | 57.215 | 49.991 | 1.00 | 133.33 | 6 |
| ATOM | 3160 | O | ALA | B | 184 | 37.857 | 56.745 | 49.109 | 1.00 | 134.76 | 8 |
| ATOM | 3161 | N | LEU | B | 185 | 36.553 | 56.454 | 50.911 | 1.00 | 148.14 | 7 |
| ATOM | 3162 | CA | LEU | B | 185 | 36.730 | 55.008 | 50.835 | 1.00 | 148.94 | 6 |
| ATOM | 3163 | CB | LEU | B | 185 | 35.855 | 54.272 | 51.850 | 1.00 | 94.82 | 6 |
| ATOM | 3164 | CG | LEU | B | 185 | 36.328 | 54.260 | 53.306 | 1.00 | 94.69 | 6 |
| ATOM | 3165 | CD1 | LEU | B | 185 | 35.483 | 53.256 | 54.075 | 1.00 | 94.46 | 6 |
| ATOM | 3166 | CD2 | LEU | B | 185 | 37.801 | 53.882 | 53.387 | 1.00 | 93.85 | 6 |
| ATOM | 3167 | C | LEU | B | 185 | 36.304 | 54.652 | 49.419 | 1.00 | 149.86 | 6 |
| ATOM | 3168 | O | LEU | B | 185 | 37.106 | 54.147 | 48.629 | 1.00 | 150.21 | 8 |
| ATOM | 3169 | N | GLY | B | 186 | 35.041 | 54.921 | 49.093 | 1.00 | 67.38 | 7 |
| ATOM | 3170 | CA | GLY | B | 186 | 34.596 | 54.663 | 47.741 | 1.00 | 68.06 | 6 |
| ATOM | 3171 | C | GLY | B | 186 | 35.455 | 55.625 | 46.944 | 1.00 | 68.15 | 6 |
| ATOM | 3172 | O | GLY | B | 186 | 35.579 | 56.778 | 47.356 | 1.00 | 68.60 | 8 |
| ATOM | 3173 | N | GLN | B | 187 | 36.064 | 55.171 | 45.847 | 1.00 | 110.85 | 7 |
| ATOM | 3174 | CA | GLN | B | 187 | 36.927 | 56.015 | 45.008 | 1.00 | 110.89 | 6 |
| ATOM | 3175 | CB | GLN | B | 187 | 36.852 | 55.563 | 43.549 | 1.00 | 71.69 | 6 |
| ATOM | 3176 | CG | GLN | B | 187 | 37.833 | 56.277 | 42.633 | 1.00 | 70.62 | 6 |
| ATOM | 3177 | CD | GLN | B | 187 | 37.348 | 57.617 | 42.111 | 1.00 | 70.79 | 6 |
| ATOM | 3178 | OE1 | GLN | B | 187 | 36.859 | 57.716 | 40.986 | 1.00 | 71.50 | 8 |
| ATOM | 3179 | NE2 | GLN | B | 187 | 37.485 | 58.654 | 42.924 | 1.00 | 70.20 | 7 |
| ATOM | 3180 | C | GLN | B | 187 | 36.619 | 57.510 | 45.069 | 1.00 | 110.45 | 6 |
| ATOM | 3181 | O | GLN | B | 187 | 37.488 | 58.312 | 45.414 | 1.00 | 110.76 | 8 |
| ATOM | 3182 | N | ARG | B | 188 | 35.394 | 57.877 | 44.694 | 1.00 | 83.21 | 7 |
| ATOM | 3183 | CA | ARG | B | 188 | 34.936 | 59.265 | 44.715 | 1.00 | 82.05 | 6 |
| ATOM | 3184 | CB | ARG | B | 188 | 33.412 | 59.298 | 44.738 | 1.00 | 138.62 | 6 |
| ATOM | 3185 | CG | ARG | B | 188 | 32.820 | 60.676 | 44.639 | 1.00 | 139.12 | 6 |
| ATOM | 3186 | CD | ARG | B | 188 | 31.322 | 60.625 | 44.855 | 1.00 | 139.80 | 6 |
| ATOM | 3187 | NE | ARG | B | 188 | 30.722 | 61.944 | 44.704 | 1.00 | 141.03 | 7 |
| ATOM | 3188 | CZ | ARG | B | 188 | 29.450 | 62.221 | 44.971 | 1.00 | 141.96 | 6 |
| ATOM | 3189 | NH1 | ARG | B | 188 | 28.639 | 61.266 | 45.409 | 1.00 | 143.04 | 7 |
| ATOM | 3190 | NH2 | ARG | B | 188 | 28.989 | 63.454 | 44.800 | 1.00 | 141.62 | 7 |
| ATOM | 3191 | C | ARG | B | 188 | 35.491 | 59.892 | 45.985 | 1.00 | 80.56 | 6 |
| ATOM | 3192 | O | ARG | B | 188 | 35.553 | 59.236 | 47.024 | 1.00 | 81.23 | 8 |
| ATOM | 3193 | N | THR | B | 189 | 35.878 | 61.161 | 45.915 | 1.00 | 77.04 | 7 |
| ATOM | 3194 | CA | THR | B | 189 | 36.477 | 61.828 | 47.067 | 1.00 | 74.63 | 6 |
| ATOM | 3195 | CB | THR | B | 189 | 37.717 | 62.551 | 46.638 | 1.00 | 59.96 | 6 |
| ATOM | 3196 | OG1 | THR | B | 189 | 37.354 | 63.515 | 45.645 | 1.00 | 60.93 | 8 |
| ATOM | 3197 | CG2 | THR | B | 189 | 38.713 | 61.575 | 46.050 | 1.00 | 59.93 | 6 |
| ATOM | 3198 | C | THR | B | 189 | 35.638 | 62.826 | 47.854 | 1.00 | 71.92 | 6 |
| ATOM | 3199 | O | THR | B | 189 | 36.171 | 63.526 | 48.716 | 1.00 | 72.50 | 8 |
| ATOM | 3200 | N | ASP | B | 190 | 34.341 | 62.905 | 47.576 | 1.00 | 63.78 | 7 |
| ATOM | 3201 | CA | ASP | B | 190 | 33.491 | 63.841 | 48.297 | 1.00 | 61.55 | 6 |
| ATOM | 3202 | CB | ASP | B | 190 | 32.921 | 64.882 | 47.330 | 1.00 | 77.28 | 6 |
| ATOM | 3203 | CG | ASP | B | 190 | 32.553 | 64.292 | 45.994 | 1.00 | 78.99 | 6 |
| ATOM | 3204 | OD1 | ASP | B | 190 | 33.453 | 63.761 | 45.309 | 1.00 | 79.32 | 8 |
| ATOM | 3205 | OD2 | ASP | B | 190 | 31.365 | 64.365 | 45.625 | 1.00 | 80.25 | 8 |
| ATOM | 3206 | C | ASP | B | 190 | 32.366 | 63.199 | 49.099 | 1.00 | 58.55 | 6 |
| ATOM | 3207 | O | ASP | B | 190 | 31.262 | 63.725 | 49.159 | 1.00 | 59.65 | 8 |
| ATOM | 3208 | N | LEU | B | 191 | 32.649 | 62.076 | 49.741 | 1.00 | 74.59 | 7 |
| ATOM | 3209 | CA | LEU | B | 191 | 31.628 | 61.417 | 50.530 | 1.00 | 71.86 | 6 |
| ATOM | 3210 | CB | LEU | B | 191 | 31.870 | 59.915 | 50.490 | 1.00 | 39.25 | 6 |
| ATOM | 3211 | CG | LEU | B | 191 | 31.700 | 59.458 | 49.027 | 1.00 | 38.40 | 6 |
| ATOM | 3212 | CD1 | LEU | B | 191 | 32.178 | 58.033 | 48.832 | 1.00 | 37.17 | 6 |
| ATOM | 3213 | CD2 | LEU | B | 191 | 30.238 | 59.588 | 48.622 | 1.00 | 37.38 | 6 |
| ATOM | 3214 | C | LEU | B | 191 | 31.610 | 61.981 | 51.953 | 1.00 | 70.45 | 6 |
| ATOM | 3215 | O | LEU | B | 191 | 32.562 | 62.647 | 52.357 | 1.00 | 72.17 | 8 |
| ATOM | 3216 | N | ASP | B | 192 | 30.523 | 61.740 | 52.696 | 1.00 | 39.62 | 7 |
| ATOM | 3217 | CA | ASP | B | 192 | 30.354 | 62.265 | 54.068 | 1.00 | 36.40 | 6 |
| ATOM | 3218 | CB | ASP | B | 192 | 28.898 | 62.701 | 54.282 | 1.00 | 78.82 | 6 |
| ATOM | 3219 | CG | ASP | B | 192 | 28.578 | 64.025 | 53.601 | 1.00 | 79.38 | 6 |
| ATOM | 3220 | OD1 | ASP | B | 192 | 29.164 | 64.299 | 52.532 | 1.00 | 79.92 | 8 |
| ATOM | 3221 | OD2 | ASP | B | 192 | 27.735 | 64.783 | 54.131 | 1.00 | 79.32 | 8 |
| ATOM | 3222 | C | ASP | B | 192 | 30.773 | 61.330 | 55.199 | 1.00 | 32.27 | 6 |
| ATOM | 3223 | O | ASP | B | 192 | 30.658 | 60.115 | 55.081 | 1.00 | 31.34 | 8 |
| ATOM | 3224 | N | LYS | B | 193 | 31.248 | 61.917 | 56.294 | 1.00 | 21.17 | 7 |
| ATOM | 3225 | CA | LYS | B | 193 | 31.718 | 61.163 | 57.450 | 1.00 | 18.63 | 6 |
| ATOM | 3226 | CB | LYS | B | 193 | 33.241 | 61.060 | 57.446 | 1.00 | 54.65 | 6 |
| ATOM | 3227 | CG | LYS | B | 193 | 33.820 | 60.353 | 58.652 | 1.00 | 55.49 | 6 |
| ATOM | 3228 | CD | LYS | B | 193 | 34.597 | 59.149 | 58.199 | 1.00 | 58.46 | 6 |
| ATOM | 3229 | CE | LYS | B | 193 | 35.959 | 59.038 | 58.888 | 1.00 | 60.61 | 6 |
| ATOM | 3230 | NZ | LYS | B | 193 | 36.881 | 58.045 | 58.209 | 1.00 | 62.12 | 7 |
| ATOM | 3231 | C | LYS | B | 193 | 31.264 | 61.796 | 58.757 | 1.00 | 16.86 | 6 |
| ATOM | 3232 | O | LYS | B | 193 | 32.071 | 62.340 | 59.534 | 1.00 | 15.25 | 8 |
| ATOM | 3233 | N | LEU | B | 194 | 29.960 | 61.671 | 58.985 | 1.00 | 56.76 | 7 |
| ATOM | 3234 | CA | LEU | B | 194 | 29.248 | 62.189 | 60.146 | 1.00 | 54.94 | 6 |
| ATOM | 3235 | CB | LEU | B | 194 | 28.004 | 61.332 | 60.376 | 1.00 | 26.43 | 6 |
| ATOM | 3236 | CG | LEU | B | 194 | 26.860 | 61.933 | 61.191 | 1.00 | 23.96 | 6 |
| ATOM | 3237 | CD1 | LEU | B | 194 | 26.960 | 61.449 | 62.612 | 1.00 | 23.82 | 6 |
| ATOM | 3238 | CD2 | LEU | B | 194 | 26.879 | 63.450 | 61.118 | 1.00 | 22.05 | 6 |
| ATOM | 3239 | C | LEU | B | 194 | 29.995 | 62.384 | 61.468 | 1.00 | 54.76 | 6 |
| ATOM | 3240 | O | LEU | B | 194 | 30.366 | 63.506 | 61.799 | 1.00 | 55.92 | 8 |
| ATOM | 3241 | N | THR | B | 195 | 30.211 | 61.316 | 62.227 | 1.00 | 24.80 | 7 |
| ATOM | 3242 | CA | THR | B | 195 | 30.885 | 61.427 | 63.529 | 1.00 | 24.82 | 6 |
| ATOM | 3243 | CB | THR | B | 195 | 32.305 | 62.008 | 63.430 | 1.00 | 13.87 | 6 |
| ATOM | 3244 | OG1 | THR | B | 195 | 33.111 | 61.165 | 62.607 | 1.00 | 13.87 | 8 |
| ATOM | 3245 | CG2 | THR | B | 195 | 32.929 | 62.113 | 64.826 | 1.00 | 13.87 | 6 |
| ATOM | 3246 | C | THR | B | 195 | 30.127 | 62.323 | 64.513 | 1.00 | 27.11 | 6 |
| ATOM | 3247 | O | THR | B | 195 | 29.933 | 63.510 | 64.266 | 1.00 | 27.79 | 8 |
| ATOM | 3248 | N | LEU | B | 196 | 29.710 | 61.748 | 65.634 | 1.00 | 83.35 | 7 |
| ATOM | 3249 | CA | LEU | B | 196 | 28.987 | 62.491 | 66.655 | 1.00 | 85.23 | 6 |
| ATOM | 3250 | CB | LEU | B | 196 | 27.600 | 61.889 | 66.865 | 1.00 | 37.74 | 6 |
| ATOM | 3251 | CG | LEU | B | 196 | 26.427 | 62.775 | 66.443 | 1.00 | 37.19 | 6 |
| ATOM | 3252 | CD1 | LEU | B | 196 | 26.607 | 63.276 | 65.031 | 1.00 | 35.58 | 6 |
| ATOM | 3253 | CD2 | LEU | B | 196 | 25.156 | 61.986 | 66.537 | 1.00 | 38.11 | 6 |
| ATOM | 3254 | C | LEU | B | 196 | 29.779 | 62.409 | 67.941 | 1.00 | 88.34 | 6 |
| ATOM | 3255 | O | LEU | B | 196 | 30.801 | 61.729 | 67.998 | 1.00 | 90.29 | 8 |
| ATOM | 3256 | N | ARG | B | 197 | 29.316 | 63.111 | 68.968 | 1.00 | 85.67 | 7 |
| ATOM | 3257 | CA | ARG | B | 197 | 29.977 | 63.095 | 70.267 | 1.00 | 87.64 | 6 |
| ATOM | 3258 | CB | ARG | B | 197 | 31.277 | 63.895 | 70.208 | 1.00 | 65.93 | 6 |
| ATOM | 3259 | CG | ARG | B | 197 | 31.185 | 65.241 | 69.531 | 1.00 | 68.55 | 6 |
| ATOM | 3260 | CD | ARG | B | 197 | 32.584 | 65.793 | 69.339 | 1.00 | 73.98 | 6 |
| ATOM | 3261 | NE | ARG | B | 197 | 32.618 | 67.245 | 69.442 | 1.00 | 77.56 | 7 |
| ATOM | 3262 | CZ | ARG | B | 197 | 33.720 | 67.948 | 69.682 | 1.00 | 79.42 | 6 |
| ATOM | 3263 | NH1 | ARG | B | 197 | 34.881 | 67.328 | 69.841 | 1.00 | 81.24 | 7 |
| ATOM | 3264 | NH2 | ARG | B | 197 | 33.659 | 69.269 | 69.778 | 1.00 | 79.66 | 7 |
| ATOM | 3265 | C | ARG | B | 197 | 29.020 | 63.642 | 71.314 | 1.00 | 89.08 | 6 |
| ATOM | 3266 | O | ARG | B | 197 | 28.980 | 64.840 | 71.597 | 1.00 | 90.63 | 8 |
| ATOM | 3267 | N | ILE | B | 198 | 28.251 | 62.718 | 71.880 | 1.00 | 28.99 | 7 |
| ATOM | 3268 | CA | ILE | B | 198 | 27.217 | 63.003 | 72.861 | 1.00 | 29.28 | 6 |
| ATOM | 3269 | CB | ILE | B | 198 | 26.045 | 61.980 | 72.665 | 1.00 | 51.66 | 6 |
| ATOM | 3270 | CG2 | ILE | B | 198 | 25.714 | 61.278 | 73.933 | 1.00 | 52.32 | 6 |
| ATOM | 3271 | CG1 | ILE | B | 198 | 24.805 | 62.683 | 72.114 | 1.00 | 52.64 | 6 |
| ATOM | 3272 | CD1 | ILE | B | 198 | 24.873 | 63.007 | 70.646 | 1.00 | 55.60 | 6 |
| ATOM | 3273 | C | ILE | B | 198 | 27.731 | 63.000 | 74.299 | 1.00 | 30.75 | 6 |
| ATOM | 3274 | O | ILE | B | 198 | 28.034 | 61.955 | 74.852 | 1.00 | 30.87 | 8 |
| ATOM | 3275 | N | TRP | B | 199 | 27.847 | 64.179 | 74.901 | 1.00 | 48.14 | 7 |
| ATOM | 3276 | CA | TRP | B | 199 | 28.312 | 64.280 | 76.282 | 1.00 | 50.03 | 6 |
| ATOM | 3277 | CB | TRP | B | 199 | 29.041 | 65.602 | 76.544 | 1.00 | 51.23 | 6 |
| ATOM | 3278 | CG | TRP | B | 199 | 30.115 | 65.946 | 75.560 | 1.00 | 52.27 | 6 |
| ATOM | 3279 | CD2 | TRP | B | 199 | 31.525 | 65.840 | 75.762 | 1.00 | 52.01 | 6 |
| ATOM | 3280 | CE2 | TRP | B | 199 | 32.149 | 66.243 | 74.564 | 1.00 | 51.99 | 6 |
| ATOM | 3281 | CE3 | TRP | B | 199 | 32.324 | 65.440 | 76.837 | 1.00 | 52.21 | 6 |
| ATOM | 3282 | CD1 | TRP | B | 199 | 29.946 | 66.401 | 74.282 | 1.00 | 52.95 | 6 |
| ATOM | 3283 | NE1 | TRP | B | 199 | 31.161 | 66.582 | 73.678 | 1.00 | 52.35 | 7 |
| ATOM | 3284 | CZ2 | TRP | B | 199 | 33.540 | 66.255 | 74.411 | 1.00 | 51.93 | 6 |
| ATOM | 3285 | CZ3 | TRP | B | 199 | 33.710 | 65.452 | 76.683 | 1.00 | 52.41 | 6 |
| ATOM | 3286 | CH2 | TRP | B | 199 | 34.301 | 65.856 | 75.480 | 1.00 | 51.86 | 6 |
| ATOM | 3287 | C | TRP | B | 199 | 27.076 | 64.235 | 77.158 | 1.00 | 51.99 | 6 |
| ATOM | 3288 | O | TRP | B | 199 | 26.265 | 65.164 | 77.145 | 1.00 | 52.37 | 8 |
| ATOM | 3289 | N | THR | B | 200 | 26.928 | 63.155 | 77.920 | 1.00 | 95.11 | 7 |
| ATOM | 3290 | CA | THR | B | 200 | 25.770 | 63.002 | 78.797 | 1.00 | 96.45 | 6 |
| ATOM | 3291 | CB | THR | B | 200 | 24.694 | 62.115 | 78.166 | 1.00 | 148.63 | 6 |
| ATOM | 3292 | OG1 | THR | B | 200 | 24.987 | 60.742 | 78.445 | 1.00 | 149.74 | 8 |
| ATOM | 3293 | CG2 | THR | B | 200 | 24.666 | 62.310 | 76.678 | 1.00 | 147.62 | 6 |
| ATOM | 3294 | C | THR | B | 200 | 26.150 | 62.350 | 80.113 | 1.00 | 96.31 | 6 |
| ATOM | 3295 | O | THR | B | 200 | 26.988 | 61.452 | 80.152 | 1.00 | 96.11 | 8 |
| ATOM | 3296 | N | ASP | B | 201 | 25.522 | 62.810 | 81.189 | 1.00 | 43.75 | 7 |
| ATOM | 3297 | CA | ASP | B | 201 | 25.759 | 62.247 | 82.514 | 1.00 | 44.52 | 6 |
| ATOM | 3298 | CB | ASP | B | 201 | 27.101 | 62.721 | 83.083 | 1.00 | 82.69 | 6 |
| ATOM | 3299 | CG | ASP | B | 201 | 27.651 | 61.776 | 84.145 | 1.00 | 84.02 | 6 |
| ATOM | 3300 | OD1 | ASP | B | 201 | 26.866 | 61.333 | 85.011 | 1.00 | 85.42 | 8 |
| ATOM | 3301 | OD2 | ASP | B | 201 | 28.868 | 61.480 | 84.120 | 1.00 | 84.72 | 8 |
| ATOM | 3302 | C | ASP | B | 201 | 24.615 | 62.631 | 83.446 | 1.00 | 44.46 | 6 |
| ATOM | 3303 | O | ASP | B | 201 | 24.623 | 62.293 | 84.631 | 1.00 | 44.19 | 8 |
| ATOM | 3304 | N | GLY | B | 202 | 23.624 | 63.329 | 82.898 | 1.00 | 75.55 | 7 |
| ATOM | 3305 | CA | GLY | B | 202 | 22.477 | 63.706 | 83.692 | 1.00 | 75.47 | 6 |
| ATOM | 3306 | C | GLY | B | 202 | 21.719 | 62.453 | 84.110 | 1.00 | 76.50 | 6 |
| ATOM | 3307 | O | GLY | B | 202 | 20.547 | 62.549 | 84.483 | 1.00 | 77.97 | 8 |
| ATOM | 3308 | N | SER | B | 203 | 22.392 | 61.292 | 84.035 | 1.00 | 55.59 | 7 |
| ATOM | 3309 | CA | SER | B | 203 | 21.867 | 59.957 | 84.392 | 1.00 | 54.17 | 6 |
| ATOM | 3310 | CB | SER | B | 203 | 20.356 | 59.993 | 84.676 | 1.00 | 91.62 | 6 |
| ATOM | 3311 | OG | SER | B | 203 | 19.605 | 60.381 | 83.538 | 1.00 | 90.27 | 8 |
| ATOM | 3312 | C | SER | B | 203 | 22.136 | 58.886 | 83.325 | 1.00 | 52.93 | 6 |
| ATOM | 3313 | O | SER | B | 203 | 23.059 | 58.092 | 83.461 | 1.00 | 53.08 | 8 |
| ATOM | 3314 | N | VAL | B | 204 | 21.309 | 58.875 | 82.277 | 1.00 | 69.60 | 7 |
| ATOM | 3315 | CA | VAL | B | 204 | 21.390 | 57.927 | 81.154 | 1.00 | 68.03 | 6 |
| ATOM | 3316 | CB | VAL | B | 204 | 20.338 | 58.242 | 80.108 | 1.00 | 35.10 | 6 |
| ATOM | 3317 | CG1 | VAL | B | 204 | 20.400 | 57.231 | 78.991 | 1.00 | 34.10 | 6 |
| ATOM | 3318 | CG2 | VAL | B | 204 | 18.985 | 58.275 | 80.746 | 1.00 | 34.56 | 6 |
| ATOM | 3319 | C | VAL | B | 204 | 22.709 | 57.931 | 80.407 | 1.00 | 67.75 | 6 |
| ATOM | 3320 | O | VAL | B | 204 | 23.357 | 58.962 | 80.309 | 1.00 | 69.06 | 8 |
| ATOM | 3321 | N | THR | B | 205 | 23.082 | 56.793 | 79.829 | 1.00 | 49.09 | 7 |
| ATOM | 3322 | CA | THR | B | 205 | 24.340 | 56.711 | 79.093 | 1.00 | 48.85 | 6 |
| ATOM | 3323 | CB | THR | B | 205 | 24.767 | 55.263 | 78.850 | 1.00 | 77.86 | 6 |
| ATOM | 3324 | OG1 | THR | B | 205 | 24.007 | 54.721 | 77.768 | 1.00 | 79.05 | 8 |
| ATOM | 3325 | CG2 | THR | B | 205 | 24.542 | 54.429 | 80.092 | 1.00 | 78.88 | 6 |
| ATOM | 3326 | C | THR | B | 205 | 24.225 | 57.400 | 77.737 | 1.00 | 49.21 | 6 |
| ATOM | 3327 | O | THR | B | 205 | 23.131 | 57.794 | 77.327 | 1.00 | 49.54 | 8 |
| ATOM | 3328 | N | PRO | B | 206 | 25.361 | 57.556 | 77.023 | 1.00 | 46.13 | 7 |
| ATOM | 3329 | CD | PRO | B | 206 | 26.729 | 57.238 | 77.468 | 1.00 | 48.07 | 6 |
| ATOM | 3330 | CA | PRO | B | 206 | 25.402 | 58.200 | 75.707 | 1.00 | 45.85 | 6 |
| ATOM | 3331 | CB | PRO | B | 206 | 26.904 | 58.299 | 75.415 | 1.00 | 48.63 | 6 |
| ATOM | 3332 | CG | PRO | B | 206 | 27.534 | 58.294 | 76.752 | 1.00 | 48.13 | 6 |
| ATOM | 3333 | C | PRO | B | 206 | 24.674 | 57.407 | 74.626 | 1.00 | 47.15 | 6 |
| ATOM | 3334 | O | PRO | B | 206 | 23.585 | 57.780 | 74.189 | 1.00 | 46.48 | 8 |
| ATOM | 3335 | N | LEU | B | 207 | 25.304 | 56.318 | 74.191 | 1.00 | 76.94 | 7 |
| ATOM | 3336 | CA | LEU | B | 207 | 24.746 | 55.460 | 73.155 | 1.00 | 77.75 | 6 |
| ATOM | 3337 | CB | LEU | B | 207 | 25.444 | 54.099 | 73.169 | 1.00 | 101.16 | 6 |
| ATOM | 3338 | CG | LEU | B | 207 | 25.120 | 53.141 | 72.022 | 1.00 | 101.47 | 6 |
| ATOM | 3339 | CD1 | LEU | B | 207 | 25.360 | 53.807 | 70.671 | 1.00 | 101.74 | 6 |
| ATOM | 3340 | CD2 | LEU | B | 207 | 25.988 | 51.910 | 72.173 | 1.00 | 101.71 | 6 |
| ATOM | 3341 | C | LEU | B | 207 | 23.252 | 55.303 | 73.390 | 1.00 | 78.54 | 6 |
| ATOM | 3342 | O | LEU | B | 207 | 22.470 | 55.176 | 72.446 | 1.00 | 78.25 | 8 |
| ATOM | 3343 | N | GLU | B | 208 | 22.864 | 55.323 | 74.660 | 1.00 | 60.01 | 7 |
| ATOM | 3344 | CA | GLU | B | 208 | 21.461 | 55.218 | 75.023 | 1.00 | 61.02 | 6 |
| ATOM | 3345 | CB | GLU | B | 208 | 21.302 | 55.117 | 76.546 | 1.00 | 128.03 | 6 |
| ATOM | 3346 | CG | GLU | B | 208 | 21.543 | 53.714 | 77.097 | 1.00 | 131.02 | 6 |
| ATOM | 3347 | CD | GLU | B | 208 | 21.287 | 53.608 | 78.591 | 1.00 | 133.34 | 6 |
| ATOM | 3348 | OE1 | GLU | B | 208 | 20.215 | 54.062 | 79.041 | 1.00 | 134.35 | 8 |
| ATOM | 3349 | OE2 | GLU | B | 208 | 22.149 | 53.060 | 79.315 | 1.00 | 134.47 | 8 |
| ATOM | 3350 | C | GLU | B | 208 | 20.742 | 56.458 | 74.497 | 1.00 | 60.26 | 6 |
| ATOM | 3351 | O | GLU | B | 208 | 19.750 | 56.360 | 73.769 | 1.00 | 58.68 | 8 |
| ATOM | 3352 | N | ALA | B | 209 | 21.249 | 57.625 | 74.865 | 1.00 | 65.53 | 7 |
| ATOM | 3353 | CA | ALA | B | 209 | 20.653 | 58.857 | 74.402 | 1.00 | 65.98 | 6 |
| ATOM | 3354 | CB | ALA | B | 209 | 21.552 | 60.033 | 74.735 | 1.00 | 16.71 | 6 |
| ATOM | 3355 | C | ALA | B | 209 | 20.453 | 58.740 | 72.891 | 1.00 | 66.92 | 6 |
| ATOM | 3356 | O | ALA | B | 209 | 19.319 | 58.605 | 72.431 | 1.00 | 67.52 | 8 |
| ATOM | 3357 | N | LEU | B | 210 | 21.543 | 58.756 | 72.123 | 1.00 | 73.15 | 7 |
| ATOM | 3358 | CA | LEU | B | 210 | 21.443 | 58.668 | 70.662 | 1.00 | 73.45 | 6 |
| ATOM | 3359 | CB | LEU | B | 210 | 22.777 | 58.207 | 70.051 | 1.00 | 36.69 | 6 |
| ATOM | 3360 | CG | LEU | B | 210 | 22.693 | 57.718 | 68.591 | 1.00 | 36.68 | 6 |
| ATOM | 3361 | CD1 | LEU | B | 210 | 22.266 | 58.832 | 67.646 | 1.00 | 36.19 | 6 |
| ATOM | 3362 | CD2 | LEU | B | 210 | 24.030 | 57.169 | 68.179 | 1.00 | 37.72 | 6 |
| ATOM | 3363 | C | LEU | B | 210 | 20.312 | 57.752 | 70.178 | 1.00 | 73.95 | 6 |
| ATOM | 3364 | O | LEU | B | 210 | 19.323 | 58.223 | 69.611 | 1.00 | 73.49 | 8 |
| ATOM | 3365 | N | ASN | B | 211 | 20.464 | 56.450 | 70.414 | 1.00 | 95.72 | 7 |
| ATOM | 3366 | CA | ASN | B | 211 | 19.475 | 55.452 | 70.005 | 1.00 | 94.91 | 6 |
| ATOM | 3367 | CB | ASN | B | 211 | 19.789 | 54.099 | 70.646 | 1.00 | 47.61 | 6 |
| ATOM | 3368 | CG | ASN | B | 211 | 20.941 | 53.388 | 69.969 | 1.00 | 48.99 | 6 |
| ATOM | 3369 | OD1 | ASN | B | 211 | 20.855 | 53.047 | 68.782 | 1.00 | 49.54 | 8 |
| ATOM | 3370 | ND2 | ASN | B | 211 | 22.025 | 53.156 | 70.711 | 1.00 | 49.80 | 7 |
| ATOM | 3371 | C | ASN | B | 211 | 18.065 | 55.862 | 70.378 | 1.00 | 94.05 | 6 |
| ATOM | 3372 | O | ASN | B | 211 | 17.183 | 55.935 | 69.524 | 1.00 | 94.10 | 8 |
| ATOM | 3373 | N | GLN | B | 212 | 17.863 | 56.119 | 71.666 | 1.00 | 48.75 | 7 |
| ATOM | 3374 | CA | GLN | B | 212 | 16.566 | 56.525 | 72.181 | 1.00 | 47.49 | 6 |
| ATOM | 3375 | CB | GLN | B | 212 | 16.712 | 56.943 | 73.643 | 1.00 | 74.54 | 6 |
| ATOM | 3376 | CG | GLN | B | 212 | 15.407 | 57.083 | 74.394 | 1.00 | 76.74 | 6 |
| ATOM | 3377 | CD | GLN | B | 212 | 15.620 | 57.235 | 75.889 | 1.00 | 78.20 | 6 |
| ATOM | 3378 | OE1 | GLN | B | 212 | 16.241 | 56.384 | 76.535 | 1.00 | 79.21 | 8 |
| ATOM | 3379 | NE2 | GLN | B | 212 | 15.099 | 58.318 | 76.450 | 1.00 | 78.87 | 7 |
| ATOM | 3380 | C | GLN | B | 212 | 16.011 | 57.678 | 71.342 | 1.00 | 46.38 | 6 |
| ATOM | 3381 | O | GLN | B | 212 | 14.860 | 57.645 | 70.908 | 1.00 | 46.06 | 8 |
| ATOM | 3382 | N | ALA | B | 213 | 16.840 | 58.690 | 71.104 | 1.00 | 58.18 | 7 |
| ATOM | 3383 | CA | ALA | B | 213 | 16.434 | 59.836 | 70.304 | 1.00 | 56.70 | 6 |
| ATOM | 3384 | CB | ALA | B | 213 | 17.506 | 60.900 | 70.344 | 1.00 | 101.95 | 6 |
| ATOM | 3385 | C | ALA | B | 213 | 16.183 | 59.399 | 68.870 | 1.00 | 56.54 | 6 |
| ATOM | 3386 | O | ALA | B | 213 | 15.253 | 59.886 | 68.229 | 1.00 | 56.71 | 8 |
| ATOM | 3387 | N | VAL | B | 214 | 17.020 | 58.478 | 68.380 | 1.00 | 57.27 | 7 |
| ATOM | 3388 | CA | VAL | B | 214 | 16.911 | 57.937 | 67.015 | 1.00 | 55.99 | 6 |
| ATOM | 3389 | CB | VAL | B | 214 | 17.914 | 56.784 | 66.758 | 1.00 | 38.97 | 6 |
| ATOM | 3390 | CG1 | VAL | B | 214 | 17.831 | 56.354 | 65.291 | 1.00 | 40.50 | 6 |
| ATOM | 3391 | CG2 | VAL | B | 214 | 19.336 | 57.210 | 67.130 | 1.00 | 39.20 | 6 |
| ATOM | 3392 | C | VAL | B | 214 | 15.519 | 57.359 | 66.847 | 1.00 | 55.21 | 6 |
| ATOM | 3393 | O | VAL | B | 214 | 14.976 | 57.258 | 65.748 | 1.00 | 54.40 | 8 |
| ATOM | 3394 | N | ALA | B | 215 | 14.963 | 56.955 | 67.972 | 1.00 | 35.62 | 7 |
| ATOM | 3395 | CA | ALA | B | 215 | 13.641 | 56.403 | 68.008 | 1.00 | 34.92 | 6 |
| ATOM | 3396 | CB | ALA | B | 215 | 13.494 | 55.553 | 69.237 | 1.00 | 13.87 | 6 |
| ATOM | 3397 | C | ALA | B | 215 | 12.692 | 57.581 | 68.064 | 1.00 | 34.79 | 6 |
| ATOM | 3398 | O | ALA | B | 215 | 11.673 | 57.605 | 67.394 | 1.00 | 35.53 | 8 |
| ATOM | 3399 | N | ILE | B | 216 | 13.051 | 58.568 | 68.873 | 1.00 | 37.58 | 7 |
| ATOM | 3400 | CA | ILE | B | 216 | 12.229 | 59.760 | 69.043 | 1.00 | 38.65 | 6 |
| ATOM | 3401 | CB | ILE | B | 216 | 12.945 | 60.781 | 69.947 | 1.00 | 58.19 | 6 |
| ATOM | 3402 | CG2 | ILE | B | 216 | 11.991 | 61.919 | 70.298 | 1.00 | 59.13 | 6 |
| ATOM | 3403 | CG1 | ILE | B | 216 | 13.441 | 60.079 | 71.212 | 1.00 | 57.58 | 6 |
| ATOM | 3404 | CD1 | ILE | B | 216 | 14.218 | 60.959 | 72.142 | 1.00 | 58.61 | 6 |
| ATOM | 3405 | C | ILE | B | 216 | 11.891 | 60.414 | 67.699 | 1.00 | 39.48 | 6 |
| ATOM | 3406 | O | ILE | B | 216 | 10.762 | 60.881 | 67.482 | 1.00 | 38.93 | 8 |
| ATOM | 3407 | N | LEU | B | 217 | 12.877 | 60.446 | 66.806 | 1.00 | 55.28 | 7 |
| ATOM | 3408 | CA | LEU | B | 217 | 12.700 | 61.025 | 65.481 | 1.00 | 56.43 | 6 |
| ATOM | 3409 | CB | LEU | B | 217 | 14.066 | 61.305 | 64.855 | 1.00 | 34.79 | 6 |
| ATOM | 3410 | CG | LEU | B | 217 | 14.067 | 61.729 | 63.387 | 1.00 | 34.72 | 6 |
| ATOM | 3411 | CD1 | LEU | B | 217 | 13.484 | 63.118 | 63.280 | 1.00 | 33.77 | 6 |
| ATOM | 3412 | CD2 | LEU | B | 217 | 15.478 | 61.688 | 62.826 | 1.00 | 33.56 | 6 |
| ATOM | 3413 | C | LEU | B | 217 | 11.915 | 60.041 | 64.606 | 1.00 | 58.48 | 6 |
| ATOM | 3414 | O | LEU | B | 217 | 10.955 | 60.420 | 63.931 | 1.00 | 58.68 | 8 |
| ATOM | 3415 | N | LYS | B | 218 | 12.340 | 58.775 | 64.635 | 1.00 | 79.73 | 7 |
| ATOM | 3416 | CA | LYS | B | 218 | 11.715 | 57.689 | 63.875 | 1.00 | 80.04 | 6 |
| ATOM | 3417 | CB | LYS | B | 218 | 12.239 | 56.347 | 64.384 | 1.00 | 60.98 | 6 |
| ATOM | 3418 | CG | LYS | B | 218 | 13.069 | 55.534 | 63.410 | 1.00 | 61.97 | 6 |
| ATOM | 3419 | CD | LYS | B | 218 | 13.516 | 54.221 | 64.104 | 1.00 | 64.17 | 6 |
| ATOM | 3420 | CE | LYS | B | 218 | 14.311 | 53.257 | 63.198 | 1.00 | 65.47 | 6 |
| ATOM | 3421 | NZ | LYS | B | 218 | 14.927 | 52.116 | 63.964 | 1.00 | 65.03 | 7 |
| ATOM | 3422 | C | LYS | B | 218 | 10.191 | 57.722 | 64.026 | 1.00 | 81.14 | 6 |
| ATOM | 3423 | O | LYS | B | 218 | 9.468 | 57.840 | 63.040 | 1.00 | 81.66 | 8 |
| ATOM | 3424 | N | GLU | B | 219 | 9.720 | 57.609 | 65.268 | 1.00 | 75.84 | 7 |
| ATOM | 3425 | CA | GLU | B | 219 | 8.295 | 57.626 | 65.579 | 1.00 | 77.32 | 6 |
| ATOM | 3426 | CB | GLU | B | 219 | 8.078 | 57.631 | 67.092 | 1.00 | 140.14 | 6 |
| ATOM | 3427 | CG | GLU | B | 219 | 6.668 | 58.061 | 67.478 | 1.00 | 143.68 | 6 |
| ATOM | 3428 | CD | GLU | B | 219 | 6.462 | 58.174 | 68.971 | 1.00 | 145.94 | 6 |
| ATOM | 3429 | OE1 | GLU | B | 219 | 7.328 | 58.769 | 69.650 | 1.00 | 146.59 | 8 |
| ATOM | 3430 | OE2 | GLU | B | 219 | 5.423 | 57.679 | 69.462 | 1.00 | 147.42 | 8 |
| ATOM | 3431 | C | GLU | B | 219 | 7.556 | 58.819 | 64.977 | 1.00 | 78.30 | 6 |
| ATOM | 3432 | O | GLU | B | 219 | 6.477 | 58.659 | 64.396 | 1.00 | 78.84 | 8 |
| ATOM | 3433 | N | HIS | B | 220 | 8.121 | 60.014 | 65.138 | 1.00 | 66.20 | 7 |
| ATOM | 3434 | CA | HIS | B | 220 | 7.504 | 61.227 | 64.609 | 1.00 | 66.15 | 6 |
| ATOM | 3435 | CB | HIS | B | 220 | 8.338 | 62.448 | 64.994 | 1.00 | 105.59 | 6 |
| ATOM | 3436 | CG | HIS | B | 220 | 8.015 | 62.991 | 66.349 | 1.00 | 108.40 | 6 |
| ATOM | 3437 | CD2 | HIS | B | 220 | 8.405 | 62.596 | 67.584 | 1.00 | 109.60 | 6 |
| ATOM | 3438 | ND1 | HIS | B | 220 | 7.157 | 64.054 | 66.540 | 1.00 | 109.83 | 7 |
| ATOM | 3439 | CE1 | HIS | B | 220 | 7.034 | 64.291 | 67.834 | 1.00 | 110.63 | 6 |
| ATOM | 3440 | NE2 | HIS | B | 220 | 7.782 | 63.420 | 68.489 | 1.00 | 111.13 | 7 |
| ATOM | 3441 | C | HIS | B | 220 | 7.343 | 61.148 | 63.102 | 1.00 | 65.62 | 6 |
| ATOM | 3442 | O | HIS | B | 220 | 6.311 | 61.534 | 62.561 | 1.00 | 65.51 | 8 |
| ATOM | 3443 | N | LEU | B | 221 | 8.370 | 60.640 | 62.429 | 1.00 | 64.25 | 7 |
| ATOM | 3444 | CA | LEU | B | 221 | 8.337 | 60.490 | 60.983 | 1.00 | 62.89 | 6 |
| ATOM | 3445 | CB | LEU | B | 221 | 9.520 | 59.655 | 60.508 | 1.00 | 40.10 | 6 |
| ATOM | 3446 | CG | LEU | B | 221 | 10.733 | 60.492 | 60.127 | 1.00 | 39.46 | 6 |
| ATOM | 3447 | CD1 | LEU | B | 221 | 12.014 | 59.665 | 60.231 | 1.00 | 38.63 | 6 |
| ATOM | 3448 | CD2 | LEU | B | 221 | 10.499 | 61.061 | 58.725 | 1.00 | 40.35 | 6 |
| ATOM | 3449 | C | LEU | B | 221 | 7.057 | 59.800 | 60.598 | 1.00 | 62.98 | 6 |
| ATOM | 3450 | O | LEU | B | 221 | 6.352 | 60.252 | 59.706 | 1.00 | 63.03 | 8 |
| ATOM | 3451 | N | ASN | B | 222 | 6.767 | 58.698 | 61.281 | 1.00 | 80.11 | 7 |
| ATOM | 3452 | CA | ASN | B | 222 | 5.561 | 57.927 | 61.033 | 1.00 | 80.71 | 6 |
| ATOM | 3453 | CB | ASN | B | 222 | 5.176 | 57.136 | 62.284 | 1.00 | 106.39 | 6 |
| ATOM | 3454 | CG | ASN | B | 222 | 6.127 | 55.993 | 62.568 | 1.00 | 108.39 | 6 |
| ATOM | 3455 | OD1 | ASN | B | 222 | 6.008 | 55.318 | 63.591 | 1.00 | 109.32 | 8 |
| ATOM | 3456 | ND2 | ASN | B | 222 | 7.072 | 55.761 | 61.660 | 1.00 | 108.36 | 7 |
| ATOM | 3457 | C | ASN | B | 222 | 4.411 | 58.842 | 60.642 | 1.00 | 80.66 | 6 |
| ATOM | 3458 | O | ASN | B | 222 | 3.870 | 58.734 | 59.542 | 1.00 | 81.98 | 8 |
| ATOM | 3459 | N | TYR | B | 223 | 4.057 | 59.744 | 61.555 | 1.00 | 63.38 | 7 |
| ATOM | 3460 | CA | TYR | B | 223 | 2.974 | 60.706 | 61.371 | 1.00 | 63.46 | 6 |
| ATOM | 3461 | CB | TYR | B | 223 | 3.332 | 61.991 | 62.111 | 1.00 | 60.86 | 6 |
| ATOM | 3462 | CG | TYR | B | 223 | 3.569 | 61.736 | 63.585 | 1.00 | 60.10 | 6 |
| ATOM | 3463 | CD1 | TYR | B | 223 | 4.374 | 62.577 | 64.344 | 1.00 | 60.05 | 6 |
| ATOM | 3464 | CE1 | TYR | B | 223 | 4.605 | 62.326 | 65.696 | 1.00 | 59.49 | 6 |
| ATOM | 3465 | CD2 | TYR | B | 223 | 2.993 | 60.637 | 64.217 | 1.00 | 60.08 | 6 |
| ATOM | 3466 | CE2 | TYR | B | 223 | 3.214 | 60.379 | 65.560 | 1.00 | 60.21 | 6 |
| ATOM | 3467 | CZ | TYR | B | 223 | 4.022 | 61.224 | 66.297 | 1.00 | 59.74 | 6 |
| ATOM | 3468 | OH | TYR | B | 223 | 4.248 | 60.951 | 67.631 | 1.00 | 59.35 | 8 |
| ATOM | 3469 | C | TYR | B | 223 | 2.646 | 60.987 | 59.913 | 1.00 | 64.43 | 6 |
| ATOM | 3470 | O | TYR | B | 223 | 1.480 | 61.164 | 59.558 | 1.00 | 64.54 | 8 |
| ATOM | 3471 | N | PHE | B | 224 | 3.678 | 61.022 | 59.074 | 1.00 | 73.75 | 7 |
| ATOM | 3472 | CA | PHE | B | 224 | 3.502 | 61.238 | 57.645 | 1.00 | 74.89 | 6 |
| ATOM | 3473 | CB | PHE | B | 224 | 4.815 | 61.699 | 56.983 | 1.00 | 86.72 | 6 |
| ATOM | 3474 | CG | PHE | B | 224 | 5.376 | 62.988 | 57.543 | 1.00 | 87.15 | 6 |
| ATOM | 3475 | CD1 | PHE | B | 224 | 6.108 | 62.992 | 58.728 | 1.00 | 87.88 | 6 |
| ATOM | 3476 | CD2 | PHE | B | 224 | 5.171 | 64.197 | 56.885 | 1.00 | 86.49 | 6 |
| ATOM | 3477 | CE1 | PHE | B | 224 | 6.624 | 64.174 | 59.245 | 1.00 | 87.12 | 6 |
| ATOM | 3478 | CE2 | PHE | B | 224 | 5.684 | 65.385 | 57.398 | 1.00 | 85.69 | 6 |
| ATOM | 3479 | CZ | PHE | B | 224 | 6.410 | 65.373 | 58.578 | 1.00 | 86.13 | 6 |
| ATOM | 3480 | C | PHE | B | 224 | 3.061 | 59.897 | 57.053 | 1.00 | 76.36 | 6 |
| ATOM | 3481 | O | PHE | B | 224 | 3.744 | 59.311 | 56.211 | 1.00 | 76.67 | 8 |
| ATOM | 3482 | N | ALA | B | 225 | 1.916 | 59.413 | 57.523 | 1.00 | 80.05 | 7 |
| ATOM | 3483 | CA | ALA | B | 225 | 1.360 | 58.149 | 57.059 | 1.00 | 81.59 | 6 |
| ATOM | 3484 | CB | ALA | B | 225 | 1.234 | 57.174 | 58.225 | 1.00 | 74.53 | 6 |
| ATOM | 3485 | C | ALA | B | 225 | −0.001 | 58.365 | 56.407 | 1.00 | 83.07 | 6 |
| ATOM | 3486 | O | ALA | B | 225 | −0.103 | 58.491 | 55.188 | 1.00 | 83.49 | 8 |
| ATOM | 3487 | N | ASN | B | 226 | −1.049 | 58.410 | 57.220 | 1.00 | 69.91 | 7 |
| ATOM | 3488 | CA | ASN | B | 226 | −2.390 | 58.608 | 56.689 | 1.00 | 72.09 | 6 |
| ATOM | 3489 | CB | ASN | B | 226 | −3.405 | 57.834 | 57.525 | 1.00 | 91.37 | 6 |
| ATOM | 3490 | CG | ASN | B | 226 | −3.139 | 56.343 | 57.519 | 1.00 | 92.71 | 6 |
| ATOM | 3491 | OD1 | ASN | B | 226 | −3.209 | 55.690 | 56.476 | 1.00 | 91.90 | 8 |
| ATOM | 3492 | ND2 | ASN | B | 226 | −2.828 | 55.796 | 58.687 | 1.00 | 93.91 | 7 |
| ATOM | 3493 | C | ASN | B | 226 | −2.734 | 60.088 | 56.670 | 1.00 | 73.36 | 6 |
| ATOM | 3494 | O | ASN | B | 226 | −2.610 | 60.771 | 57.683 | 1.00 | 74.07 | 8 |
| ATOM | 3495 | N | PRO | B | 227 | −3.157 | 60.602 | 55.502 | 1.00 | 115.46 | 7 |
| ATOM | 3496 | CD | PRO | B | 227 | −2.811 | 59.980 | 54.207 | 1.00 | 39.04 | 6 |
| ATOM | 3497 | CA | PRO | B | 227 | −3.522 | 62.013 | 55.317 | 1.00 | 116.36 | 6 |
| ATOM | 3498 | CB | PRO | B | 227 | −2.727 | 62.390 | 54.077 | 1.00 | 39.80 | 6 |
| ATOM | 3499 | CG | PRO | B | 227 | −2.919 | 61.146 | 53.223 | 1.00 | 39.44 | 6 |
| ATOM | 3500 | C | PRO | B | 227 | −5.008 | 62.361 | 55.141 | 1.00 | 118.18 | 6 |
| ATOM | 3501 | O | PRO | B | 227 | −5.859 | 61.494 | 54.963 | 1.00 | 118.42 | 8 |
| ATOM | 3502 | N | GLU | B | 228 | −5.294 | 63.656 | 55.199 | 1.00 | 106.47 | 7 |
| ATOM | 3503 | CA | GLU | B | 228 | −6.636 | 64.181 | 54.991 | 1.00 | 108.65 | 6 |
| ATOM | 3504 | CB | GLU | B | 228 | −7.302 | 64.555 | 56.319 | 1.00 | 173.49 | 6 |
| ATOM | 3505 | CG | GLU | B | 228 | −8.736 | 65.043 | 56.146 | 1.00 | 177.01 | 6 |
| ATOM | 3506 | CD | GLU | B | 228 | −9.474 | 65.203 | 57.459 | 1.00 | 179.13 | 6 |
| ATOM | 3507 | OE1 | GLU | B | 228 | −9.021 | 65.999 | 58.309 | 1.00 | 181.08 | 8 |
| ATOM | 3508 | OE2 | GLU | B | 228 | −10.514 | 64.534 | 57.635 | 1.00 | 180.01 | 8 |
| ATOM | 3509 | C | GLU | B | 228 | −6.406 | 65.425 | 54.128 | 1.00 | 110.13 | 6 |
| ATOM | 3510 | O | GLU | B | 228 | −6.091 | 66.501 | 54.635 | 1.00 | 110.94 | 8 |
| ATOM | 3511 | N | ALA | B | 229 | −6.561 | 65.265 | 52.818 | 1.00 | 60.59 | 7 |
| ATOM | 3512 | CA | ALA | B | 229 | −6.311 | 66.344 | 51.876 | 1.00 | 61.24 | 6 |
| ATOM | 3513 | CB | ALA | B | 229 | −5.424 | 65.825 | 50.764 | 1.00 | 14.58 | 6 |
| ATOM | 3514 | C | ALA | B | 229 | −7.527 | 67.025 | 51.266 | 1.00 | 62.40 | 6 |
| ATOM | 3515 | O | ALA | B | 229 | −8.670 | 66.677 | 51.551 | 1.00 | 62.49 | 8 |
| ATOM | 3516 | N | SER | B | 230 | −7.236 | 68.005 | 50.413 | 1.00 | 80.99 | 7 |
| ATOM | 3517 | CA | SER | B | 230 | −8.231 | 68.789 | 49.690 | 1.00 | 83.43 | 6 |
| ATOM | 3518 | CB | SER | B | 230 | −8.273 | 70.220 | 50.237 | 1.00 | 134.32 | 6 |
| ATOM | 3519 | OG | SER | B | 230 | −8.505 | 70.230 | 51.635 | 1.00 | 135.82 | 8 |
| ATOM | 3520 | C | SER | B | 230 | −7.766 | 68.805 | 48.234 | 1.00 | 84.59 | 6 |
| ATOM | 3521 | O | SER | B | 230 | −6.569 | 68.843 | 47.973 | 1.00 | 84.28 | 8 |
| ATOM | 3522 | N | SER | B | 231 | −8.703 | 68.782 | 47.291 | 1.00 | 152.80 | 7 |
| ATOM | 3523 | CA | SER | B | 231 | −8.360 | 68.774 | 45.867 | 1.00 | 154.78 | 6 |
| ATOM | 3524 | CB | SER | B | 231 | −9.544 | 68.232 | 45.056 | 1.00 | 208.87 | 6 |
| ATOM | 3525 | OG | SER | B | 231 | −10.734 | 68.948 | 45.338 | 1.00 | 208.87 | 8 |
| ATOM | 3526 | C | SER | B | 231 | −7.933 | 70.141 | 45.320 | 1.00 | 155.53 | 6 |
| ATOM | 3527 | O | SER | B | 231 | −8.412 | 70.526 | 44.231 | 1.00 | 155.94 | 8 |
| ATOM | 3528 | OXT | SER | B | 231 | −7.102 | 70.803 | 45.978 | 1.00 | 208.87 | 8 |
| ATOM | 3529 | CB | ALA | C | 2 | 8.362 | 95.868 | 32.510 | 1.00 | 125.97 | 6 |
| ATOM | 3530 | C | ALA | C | 2 | 10.376 | 97.139 | 31.725 | 1.00 | 93.91 | 6 |
| ATOM | 3531 | O | ALA | C | 2 | 11.120 | 96.421 | 32.394 | 1.00 | 93.90 | 8 |
| ATOM | 3532 | N | ALA | C | 2 | 8.926 | 95.898 | 30.107 | 1.00 | 94.38 | 7 |
| ATOM | 3533 | CA | ALA | C | 2 | 8.955 | 96.695 | 31.369 | 1.00 | 94.22 | 6 |
| ATOM | 3534 | N | ILE | C | 3 | 10.744 | 98.330 | 31.276 | 1.00 | 119.26 | 7 |
| ATOM | 3535 | CA | ILE | C | 3 | 12.067 | 98.868 | 31.547 | 1.00 | 117.83 | 6 |
| ATOM | 3536 | CB | ILE | C | 3 | 12.862 | 99.056 | 30.224 | 1.00 | 58.24 | 6 |
| ATOM | 3537 | CG2 | ILE | C | 3 | 14.219 | 99.663 | 30.496 | 1.00 | 58.03 | 6 |
| ATOM | 3538 | CG1 | ILE | C | 3 | 13.034 | 97.707 | 29.519 | 1.00 | 58.36 | 6 |
| ATOM | 3539 | CD1 | ILE | C | 3 | 14.019 | 96.765 | 30.177 | 1.00 | 56.18 | 6 |
| ATOM | 3540 | C | ILE | C | 3 | 11.892 | 100.215 | 32.237 | 1.00 | 117.16 | 6 |
| ATOM | 3541 | O | ILE | C | 3 | 11.383 | 101.155 | 31.628 | 1.00 | 117.61 | 8 |
| ATOM | 3542 | N | LYS | C | 4 | 12.290 | 100.307 | 33.507 | 1.00 | 25.32 | 7 |
| ATOM | 3543 | CA | LYS | C | 4 | 12.161 | 101.565 | 34.243 | 1.00 | 24.08 | 6 |
| ATOM | 3544 | CB | LYS | C | 4 | 11.855 | 101.287 | 35.718 | 1.00 | 108.61 | 6 |
| ATOM | 3545 | CG | LYS | C | 4 | 11.397 | 102.522 | 36.477 | 1.00 | 111.82 | 6 |
| ATOM | 3546 | CD | LYS | C | 4 | 10.806 | 102.188 | 37.843 | 1.00 | 114.29 | 6 |
| ATOM | 3547 | CE | LYS | C | 4 | 10.245 | 103.444 | 38.512 | 1.00 | 116.05 | 6 |
| ATOM | 3548 | NZ | LYS | C | 4 | 9.624 | 103.185 | 39.844 | 1.00 | 117.65 | 7 |
| ATOM | 3549 | C | LYS | C | 4 | 13.417 | 102.440 | 34.102 | 1.00 | 22.74 | 6 |
| ATOM | 3550 | O | LYS | C | 4 | 14.514 | 102.054 | 34.485 | 1.00 | 21.59 | 8 |
| ATOM | 3551 | N | ARG | C | 5 | 13.258 | 103.622 | 33.527 | 1.00 | 40.10 | 7 |
| ATOM | 3552 | CA | ARG | C | 5 | 14.405 | 104.482 | 33.356 | 1.00 | 41.90 | 6 |
| ATOM | 3553 | CB | ARG | C | 5 | 14.312 | 105.225 | 32.019 | 1.00 | 84.10 | 6 |
| ATOM | 3554 | CG | ARG | C | 5 | 15.613 | 105.922 | 31.616 | 1.00 | 86.67 | 6 |
| ATOM | 3555 | CD | ARG | C | 5 | 15.822 | 105.907 | 30.105 | 1.00 | 88.03 | 6 |
| ATOM | 3556 | NE | ARG | C | 5 | 17.166 | 106.347 | 29.739 | 1.00 | 89.24 | 7 |
| ATOM | 3557 | CZ | ARG | C | 5 | 17.695 | 106.206 | 28.527 | 1.00 | 90.34 | 6 |
| ATOM | 3558 | NH1 | ARG | C | 5 | 16.996 | 105.635 | 27.553 | 1.00 | 91.45 | 7 |
| ATOM | 3559 | NH2 | ARG | C | 5 | 18.927 | 106.633 | 28.286 | 1.00 | 90.51 | 7 |
| ATOM | 3560 | C | ARG | C | 5 | 14.526 | 105.462 | 34.515 | 1.00 | 42.57 | 6 |
| ATOM | 3561 | O | ARG | C | 5 | 13.551 | 105.754 | 35.198 | 1.00 | 42.05 | 8 |
| ATOM | 3562 | N | PHE | C | 6 | 15.743 | 105.946 | 34.739 | 1.00 | 41.22 | 7 |
| ATOM | 3563 | CA | PHE | C | 6 | 16.053 | 106.895 | 35.804 | 1.00 | 42.96 | 6 |
| ATOM | 3564 | CB | PHE | C | 6 | 16.862 | 106.193 | 36.915 | 1.00 | 74.13 | 6 |
| ATOM | 3565 | CG | PHE | C | 6 | 16.116 | 105.090 | 37.642 | 1.00 | 74.47 | 6 |
| ATOM | 3566 | CD1 | PHE | C | 6 | 15.633 | 103.981 | 36.963 | 1.00 | 74.97 | 6 |
| ATOM | 3567 | CD2 | PHE | C | 6 | 15.898 | 105.171 | 39.013 | 1.00 | 74.02 | 6 |
| ATOM | 3568 | CE1 | PHE | C | 6 | 14.942 | 102.975 | 37.643 | 1.00 | 74.17 | 6 |
| ATOM | 3569 | CE2 | PHE | C | 6 | 15.210 | 104.172 | 39.694 | 1.00 | 72.21 | 6 |
| ATOM | 3570 | CZ | PHE | C | 6 | 14.732 | 103.076 | 39.010 | 1.00 | 72.32 | 6 |
| ATOM | 3571 | C | PHE | C | 6 | 16.950 | 107.919 | 35.121 | 1.00 | 44.27 | 6 |
| ATOM | 3572 | O | PHE | C | 6 | 17.774 | 107.508 | 34.310 | 1.00 | 44.21 | 8 |
| ATOM | 3573 | N | GLY | C | 7 | 16.817 | 109.221 | 35.408 | 1.00 | 93.24 | 7 |
| ATOM | 3574 | CA | GLY | C | 7 | 17.711 | 110.177 | 34.750 | 1.00 | 95.08 | 6 |
| ATOM | 3575 | C | GLY | C | 7 | 17.441 | 111.682 | 34.660 | 1.00 | 95.99 | 6 |
| ATOM | 3576 | O | GLY | C | 7 | 16.818 | 112.268 | 35.548 | 1.00 | 95.32 | 8 |
| ATOM | 3577 | N | ARG | C | 8 | 17.956 | 112.298 | 33.583 | 1.00 | 71.19 | 7 |
| ATOM | 3578 | CA | ARG | C | 8 | 17.813 | 113.738 | 33.283 | 1.00 | 72.07 | 6 |
| ATOM | 3579 | CB | ARG | C | 8 | 18.364 | 114.580 | 34.432 | 1.00 | 113.66 | 6 |
| ATOM | 3580 | CG | ARG | C | 8 | 17.325 | 115.062 | 35.414 | 1.00 | 115.61 | 6 |
| ATOM | 3581 | CD | ARG | C | 8 | 17.948 | 116.047 | 36.382 | 1.00 | 118.64 | 6 |
| ATOM | 3582 | NE | ARG | C | 8 | 16.997 | 116.518 | 37.381 | 1.00 | 121.32 | 7 |
| ATOM | 3583 | CZ | ARG | C | 8 | 17.309 | 117.340 | 38.377 | 1.00 | 122.59 | 6 |
| ATOM | 3584 | NH1 | ARG | C | 8 | 18.553 | 117.786 | 38.506 | 1.00 | 121.72 | 7 |
| ATOM | 3585 | NH2 | ARG | C | 8 | 16.377 | 117.713 | 39.245 | 1.00 | 124.20 | 7 |
| ATOM | 3586 | C | ARG | C | 8 | 18.469 | 114.214 | 31.958 | 1.00 | 71.82 | 6 |
| ATOM | 3587 | O | ARG | C | 8 | 19.507 | 113.707 | 31.543 | 1.00 | 71.24 | 8 |
| ATOM | 3588 | N | ILE | C | 9 | 17.844 | 115.204 | 31.319 | 1.00 | 97.66 | 7 |
| ATOM | 3589 | CA | ILE | C | 9 | 18.289 | 115.817 | 30.055 | 1.00 | 99.18 | 6 |
| ATOM | 3590 | CB | ILE | C | 9 | 19.471 | 116.816 | 30.286 | 1.00 | 113.15 | 6 |
| ATOM | 3591 | CG2 | ILE | C | 9 | 19.860 | 117.489 | 28.975 | 1.00 | 113.13 | 6 |
| ATOM | 3592 | CG1 | ILE | C | 9 | 19.043 | 117.954 | 31.221 | 1.00 | 113.50 | 6 |
| ATOM | 3593 | CD1 | ILE | C | 9 | 18.806 | 117.547 | 32.651 | 1.00 | 114.11 | 6 |
| ATOM | 3594 | C | ILE | C | 9 | 18.652 | 114.882 | 28.889 | 1.00 | 99.69 | 6 |
| ATOM | 3595 | O | ILE | C | 9 | 17.787 | 114.176 | 28.371 | 1.00 | 99.98 | 8 |
| ATOM | 3596 | N | ARG | C | 10 | 19.923 | 114.907 | 28.479 | 1.00 | 91.44 | 7 |
| ATOM | 3597 | CA | ARG | C | 10 | 20.469 | 114.115 | 27.365 | 1.00 | 91.76 | 6 |
| ATOM | 3598 | CB | ARG | C | 10 | 19.799 | 112.738 | 27.277 | 1.00 | 175.32 | 6 |
| ATOM | 3599 | CG | ARG | C | 10 | 20.491 | 111.780 | 26.317 | 1.00 | 178.69 | 6 |
| ATOM | 3600 | CD | ARG | C | 10 | 19.809 | 110.423 | 26.287 | 1.00 | 181.18 | 6 |
| ATOM | 3601 | NE | ARG | C | 10 | 20.567 | 109.457 | 25.498 | 1.00 | 183.82 | 7 |
| ATOM | 3602 | CZ | ARG | C | 10 | 20.194 | 108.196 | 25.303 | 1.00 | 185.19 | 6 |
| ATOM | 3603 | NH1 | ARG | C | 10 | 19.067 | 107.745 | 25.838 | 1.00 | 185.55 | 7 |
| ATOM | 3604 | NH2 | ARG | C | 10 | 20.950 | 107.385 | 24.575 | 1.00 | 186.22 | 7 |
| ATOM | 3605 | C | ARG | C | 10 | 20.281 | 114.875 | 26.045 | 1.00 | 91.37 | 6 |
| ATOM | 3606 | O | ARG | C | 10 | 19.738 | 114.343 | 25.074 | 1.00 | 91.78 | 8 |
| ATOM | 3607 | N | GLU | C | 11 | 20.758 | 116.119 | 26.028 | 1.00 | 37.16 | 7 |
| ATOM | 3608 | CA | GLU | C | 11 | 20.654 | 117.028 | 24.883 | 1.00 | 36.54 | 6 |
| ATOM | 3609 | CB | GLU | C | 11 | 21.407 | 118.309 | 25.202 | 1.00 | 82.90 | 6 |
| ATOM | 3610 | CG | GLU | C | 11 | 21.511 | 119.279 | 24.057 | 1.00 | 84.74 | 6 |
| ATOM | 3611 | CD | GLU | C | 11 | 22.173 | 120.560 | 24.489 | 1.00 | 86.15 | 6 |
| ATOM | 3612 | OE1 | GLU | C | 11 | 23.250 | 120.472 | 25.108 | 1.00 | 87.27 | 8 |
| ATOM | 3613 | OE2 | GLU | C | 11 | 21.624 | 121.651 | 24.221 | 1.00 | 87.25 | 8 |
| ATOM | 3614 | C | GLU | C | 11 | 21.087 | 116.547 | 23.509 | 1.00 | 36.03 | 6 |
| ATOM | 3615 | O | GLU | C | 11 | 20.621 | 117.077 | 22.505 | 1.00 | 36.52 | 8 |
| ATOM | 3616 | N | VAL | C | 12 | 21.980 | 115.563 | 23.453 | 1.00 | 63.96 | 7 |
| ATOM | 3617 | CA | VAL | C | 12 | 22.465 | 115.037 | 22.174 | 1.00 | 63.48 | 6 |
| ATOM | 3618 | CB | VAL | C | 12 | 23.871 | 115.645 | 21.828 | 1.00 | 43.42 | 6 |
| ATOM | 3619 | CG1 | VAL | C | 12 | 24.359 | 115.158 | 20.481 | 1.00 | 43.03 | 6 |
| ATOM | 3620 | CG2 | VAL | C | 12 | 23.782 | 117.160 | 21.807 | 1.00 | 41.74 | 6 |
| ATOM | 3621 | C | VAL | C | 12 | 22.525 | 113.498 | 22.181 | 1.00 | 63.52 | 6 |
| ATOM | 3622 | O | VAL | C | 12 | 21.596 | 112.838 | 22.653 | 1.00 | 64.01 | 8 |
| ATOM | 3623 | N | ILE | C | 13 | 23.614 | 112.945 | 21.651 | 1.00 | 82.06 | 7 |
| ATOM | 3624 | CA | ILE | C | 13 | 23.830 | 111.502 | 21.563 | 1.00 | 82.83 | 6 |
| ATOM | 3625 | CB | ILE | C | 13 | 23.440 | 110.967 | 20.150 | 1.00 | 46.79 | 6 |
| ATOM | 3626 | CG2 | ILE | C | 13 | 24.586 | 110.181 | 19.525 | 1.00 | 46.26 | 6 |
| ATOM | 3627 | CG1 | ILE | C | 13 | 22.197 | 110.086 | 20.244 | 1.00 | 46.68 | 6 |
| ATOM | 3628 | CD1 | ILE | C | 13 | 20.964 | 110.830 | 20.703 | 1.00 | 47.84 | 6 |
| ATOM | 3629 | C | ILE | C | 13 | 25.313 | 111.220 | 21.813 | 1.00 | 83.68 | 6 |
| ATOM | 3630 | O | ILE | C | 13 | 26.152 | 112.109 | 21.632 | 1.00 | 84.19 | 8 |
| ATOM | 3631 | N | PRO | C | 14 | 25.658 | 109.979 | 22.226 | 1.00 | 200.26 | 7 |
| ATOM | 3632 | CD | PRO | C | 14 | 24.784 | 108.804 | 22.390 | 1.00 | 143.05 | 6 |
| ATOM | 3633 | CA | PRO | C | 14 | 27.055 | 109.613 | 22.492 | 1.00 | 200.29 | 6 |
| ATOM | 3634 | CB | PRO | C | 14 | 26.958 | 108.144 | 22.909 | 1.00 | 142.05 | 6 |
| ATOM | 3635 | CG | PRO | C | 14 | 25.752 | 107.666 | 22.176 | 1.00 | 143.08 | 6 |
| ATOM | 3636 | C | PRO | C | 14 | 27.968 | 109.827 | 21.289 | 1.00 | 198.70 | 6 |
| ATOM | 3637 | O | PRO | C | 14 | 29.189 | 109.735 | 21.406 | 1.00 | 199.16 | 8 |
| ATOM | 3638 | N | LEU | C | 15 | 27.362 | 110.104 | 20.137 | 1.00 | 90.84 | 7 |
| ATOM | 3639 | CA | LEU | C | 15 | 28.091 | 110.369 | 18.901 | 1.00 | 91.40 | 6 |
| ATOM | 3640 | CB | LEU | C | 15 | 29.307 | 111.242 | 19.178 | 1.00 | 74.19 | 6 |
| ATOM | 3641 | CG | LEU | C | 15 | 30.246 | 111.448 | 17.993 | 1.00 | 73.88 | 6 |
| ATOM | 3642 | CD1 | LEU | C | 15 | 29.550 | 112.193 | 16.853 | 1.00 | 73.64 | 6 |
| ATOM | 3643 | CD2 | LEU | C | 15 | 31.437 | 112.218 | 18.495 | 1.00 | 73.28 | 6 |
| ATOM | 3644 | C | LEU | C | 15 | 28.551 | 109.146 | 18.135 | 1.00 | 91.83 | 6 |
| ATOM | 3645 | O | LEU | C | 15 | 29.665 | 108.659 | 18.333 | 1.00 | 92.24 | 8 |
| ATOM | 3646 | N | PRO | C | 16 | 27.711 | 108.653 | 17.216 | 1.00 | 77.46 | 7 |
| ATOM | 3647 | CD | PRO | C | 16 | 26.444 | 109.243 | 16.749 | 1.00 | 201.81 | 6 |
| ATOM | 3648 | CA | PRO | C | 16 | 28.062 | 107.475 | 16.421 | 1.00 | 76.68 | 6 |
| ATOM | 3649 | CB | PRO | C | 16 | 26.816 | 107.255 | 15.567 | 1.00 | 201.48 | 6 |
| ATOM | 3650 | CG | PRO | C | 16 | 26.309 | 108.651 | 15.365 | 1.00 | 202.73 | 6 |
| ATOM | 3651 | C | PRO | C | 16 | 29.312 | 107.703 | 15.579 | 1.00 | 74.58 | 6 |
| ATOM | 3652 | O | PRO | C | 16 | 29.660 | 108.831 | 15.248 | 1.00 | 73.75 | 8 |
| ATOM | 3653 | N | PRO | C | 17 | 29.999 | 106.622 | 15.222 | 1.00 | 94.18 | 7 |
| ATOM | 3654 | CD | PRO | C | 17 | 30.910 | 106.529 | 14.067 | 1.00 | 208.87 | 6 |
| ATOM | 3655 | CA | PRO | C | 17 | 29.555 | 105.294 | 15.633 | 1.00 | 92.59 | 6 |
| ATOM | 3656 | CB | PRO | C | 17 | 29.755 | 104.479 | 14.370 | 1.00 | 208.87 | 6 |
| ATOM | 3657 | CG | PRO | C | 17 | 31.069 | 105.022 | 13.882 | 1.00 | 208.87 | 6 |
| ATOM | 3658 | C | PRO | C | 17 | 30.410 | 104.778 | 16.767 | 1.00 | 88.70 | 6 |
| ATOM | 3659 | O | PRO | C | 17 | 30.400 | 103.588 | 17.055 | 1.00 | 88.26 | 8 |
| ATOM | 3660 | N | LEU | C | 18 | 31.143 | 105.676 | 17.410 | 1.00 | 55.26 | 7 |
| ATOM | 3661 | CA | LEU | C | 18 | 32.039 | 105.281 | 18.485 | 1.00 | 51.42 | 6 |
| ATOM | 3662 | CB | LEU | C | 18 | 31.421 | 104.184 | 19.356 | 1.00 | 53.94 | 6 |
| ATOM | 3663 | CG | LEU | C | 18 | 30.219 | 104.518 | 20.241 | 1.00 | 53.68 | 6 |
| ATOM | 3664 | CD1 | LEU | C | 18 | 29.655 | 103.219 | 20.819 | 1.00 | 53.23 | 6 |
| ATOM | 3665 | CD2 | LEU | C | 18 | 30.643 | 105.475 | 21.358 | 1.00 | 52.67 | 6 |
| ATOM | 3666 | C | LEU | C | 18 | 33.291 | 104.752 | 17.804 | 1.00 | 49.36 | 6 |
| ATOM | 3667 | O | LEU | C | 18 | 34.399 | 105.087 | 18.201 | 1.00 | 49.27 | 8 |
| ATOM | 3668 | N | THR | C | 19 | 33.109 | 103.920 | 16.780 | 1.00 | 20.15 | 7 |
| ATOM | 3669 | CA | THR | C | 19 | 34.236 | 103.387 | 16.017 | 1.00 | 18.27 | 6 |
| ATOM | 3670 | CB | THR | C | 19 | 33.908 | 102.070 | 15.315 | 1.00 | 40.27 | 6 |
| ATOM | 3671 | OG1 | THR | C | 19 | 32.747 | 102.248 | 14.494 | 1.00 | 40.82 | 8 |
| ATOM | 3672 | CG2 | THR | C | 19 | 33.669 | 100.974 | 16.318 | 1.00 | 40.29 | 6 |
| ATOM | 3673 | C | THR | C | 19 | 34.504 | 104.411 | 14.925 | 1.00 | 17.52 | 6 |
| ATOM | 3674 | O | THR | C | 19 | 34.690 | 104.063 | 13.759 | 1.00 | 16.43 | 8 |
| ATOM | 3675 | N | GLU | C | 20 | 34.503 | 105.678 | 15.314 | 1.00 | 36.78 | 7 |
| ATOM | 3676 | CA | GLU | C | 20 | 34.730 | 106.759 | 14.378 | 1.00 | 38.00 | 6 |
| ATOM | 3677 | CB | GLU | C | 20 | 33.761 | 107.900 | 14.677 | 1.00 | 83.62 | 6 |
| ATOM | 3678 | CG | GLU | C | 20 | 33.772 | 109.033 | 13.669 | 1.00 | 85.64 | 6 |
| ATOM | 3679 | CD | GLU | C | 20 | 33.406 | 110.357 | 14.311 | 1.00 | 87.68 | 6 |
| ATOM | 3680 | OE1 | GLU | C | 20 | 33.203 | 111.351 | 13.579 | 1.00 | 88.74 | 8 |
| ATOM | 3681 | OE2 | GLU | C | 20 | 33.335 | 110.401 | 15.560 | 1.00 | 88.07 | 8 |
| ATOM | 3682 | C | GLU | C | 20 | 36.171 | 107.256 | 14.458 | 1.00 | 37.77 | 6 |
| ATOM | 3683 | O | GLU | C | 20 | 36.635 | 107.952 | 13.547 | 1.00 | 37.06 | 8 |
| ATOM | 3684 | N | ILE | C | 21 | 36.882 | 106.917 | 15.537 | 1.00 | 33.12 | 7 |
| ATOM | 3685 | CA | ILE | C | 21 | 38.271 | 107.361 | 15.648 | 1.00 | 35.17 | 6 |
| ATOM | 3686 | CB | ILE | C | 21 | 39.008 | 106.782 | 16.864 | 1.00 | 70.71 | 6 |
| ATOM | 3687 | CG2 | ILE | C | 21 | 40.388 | 107.421 | 16.972 | 1.00 | 70.79 | 6 |
| ATOM | 3688 | CG1 | ILE | C | 21 | 38.240 | 107.095 | 18.144 | 1.00 | 72.41 | 6 |
| ATOM | 3689 | CD1 | ILE | C | 21 | 38.119 | 108.584 | 18.428 | 1.00 | 73.92 | 6 |
| ATOM | 3690 | C | ILE | C | 21 | 38.911 | 106.808 | 14.402 | 1.00 | 36.07 | 6 |
| ATOM | 3691 | O | ILE | C | 21 | 39.725 | 107.473 | 13.766 | 1.00 | 35.93 | 8 |
| ATOM | 3692 | N | GLN | C | 22 | 38.513 | 105.580 | 14.067 | 1.00 | 69.90 | 7 |
| ATOM | 3693 | CA | GLN | C | 22 | 38.978 | 104.882 | 12.874 | 1.00 | 71.08 | 6 |
| ATOM | 3694 | CB | GLN | C | 22 | 39.300 | 103.429 | 13.206 | 1.00 | 51.83 | 6 |
| ATOM | 3695 | CG | GLN | C | 22 | 40.499 | 103.265 | 14.124 | 1.00 | 51.01 | 6 |
| ATOM | 3696 | CD | GLN | C | 22 | 40.953 | 101.825 | 14.232 | 1.00 | 50.49 | 6 |
| ATOM | 3697 | OE1 | GLN | C | 22 | 40.187 | 100.959 | 14.651 | 1.00 | 51.17 | 8 |
| ATOM | 3698 | NE2 | GLN | C | 22 | 42.202 | 101.561 | 13.854 | 1.00 | 49.03 | 7 |
| ATOM | 3699 | C | GLN | C | 22 | 37.827 | 104.958 | 11.880 | 1.00 | 72.24 | 6 |
| ATOM | 3700 | O | GLN | C | 22 | 37.053 | 105.910 | 11.926 | 1.00 | 72.58 | 8 |
| ATOM | 3701 | N | VAL | C | 23 | 37.698 | 103.979 | 10.989 | 1.00 | 54.37 | 7 |
| ATOM | 3702 | CA | VAL | C | 23 | 36.595 | 104.006 | 10.031 | 1.00 | 55.27 | 6 |
| ATOM | 3703 | CB | VAL | C | 23 | 35.227 | 103.880 | 10.746 | 1.00 | 45.46 | 6 |
| ATOM | 3704 | CG1 | VAL | C | 23 | 34.080 | 104.090 | 9.748 | 1.00 | 45.92 | 6 |
| ATOM | 3705 | CG2 | VAL | C | 23 | 35.117 | 102.528 | 11.423 | 1.00 | 46.45 | 6 |
| ATOM | 3706 | C | VAL | C | 23 | 36.588 | 105.323 | 9.270 | 1.00 | 56.22 | 6 |
| ATOM | 3707 | O | VAL | C | 23 | 37.149 | 105.423 | 8.180 | 1.00 | 57.00 | 8 |
| ATOM | 3708 | N | GLU | C | 24 | 35.933 | 106.324 | 9.862 | 1.00 | 39.91 | 7 |
| ATOM | 3709 | CA | GLU | C | 24 | 35.821 | 107.663 | 9.288 | 1.00 | 41.34 | 6 |
| ATOM | 3710 | CB | GLU | C | 24 | 34.757 | 108.486 | 10.025 | 1.00 | 111.93 | 6 |
| ATOM | 3711 | CG | GLU | C | 24 | 33.323 | 108.053 | 9.752 | 1.00 | 116.16 | 6 |
| ATOM | 3712 | CD | GLU | C | 24 | 32.307 | 108.949 | 10.430 | 1.00 | 118.56 | 6 |
| ATOM | 3713 | OE1 | GLU | C | 24 | 32.302 | 110.163 | 10.148 | 1.00 | 120.21 | 8 |
| ATOM | 3714 | OE2 | GLU | C | 24 | 31.507 | 108.446 | 11.245 | 1.00 | 120.53 | 8 |
| ATOM | 3715 | C | GLU | C | 24 | 37.150 | 108.376 | 9.365 | 1.00 | 40.87 | 6 |
| ATOM | 3716 | O | GLU | C | 24 | 37.227 | 109.589 | 9.188 | 1.00 | 41.48 | 8 |
| ATOM | 3717 | N | SER | C | 25 | 38.194 | 107.609 | 9.643 | 1.00 | 48.64 | 7 |
| ATOM | 3718 | CA | SER | C | 25 | 39.533 | 108.154 | 9.722 | 1.00 | 48.38 | 6 |
| ATOM | 3719 | CB | SER | C | 25 | 40.287 | 107.593 | 10.930 | 1.00 | 73.51 | 6 |
| ATOM | 3720 | OG | SER | C | 25 | 41.621 | 108.083 | 10.983 | 1.00 | 73.78 | 8 |
| ATOM | 3721 | C | SER | C | 25 | 40.255 | 107.750 | 8.461 | 1.00 | 47.83 | 6 |
| ATOM | 3722 | O | SER | C | 25 | 40.710 | 108.593 | 7.698 | 1.00 | 47.42 | 8 |
| ATOM | 3723 | N | TYR | C | 26 | 40.346 | 106.446 | 8.243 | 1.00 | 54.43 | 7 |
| ATOM | 3724 | CA | TYR | C | 26 | 41.034 | 105.924 | 7.077 | 1.00 | 54.68 | 6 |
| ATOM | 3725 | CB | TYR | C | 26 | 40.858 | 104.404 | 7.005 | 1.00 | 49.43 | 6 |
| ATOM | 3726 | CG | TYR | C | 26 | 42.038 | 103.677 | 6.406 | 1.00 | 47.66 | 6 |
| ATOM | 3727 | CD1 | TYR | C | 26 | 43.210 | 103.498 | 7.128 | 1.00 | 46.59 | 6 |
| ATOM | 3728 | CE1 | TYR | C | 26 | 44.299 | 102.857 | 6.572 | 1.00 | 46.24 | 6 |
| ATOM | 3729 | CD2 | TYR | C | 26 | 41.990 | 103.191 | 5.109 | 1.00 | 47.36 | 6 |
| ATOM | 3730 | CE2 | TYR | C | 26 | 43.077 | 102.550 | 4.549 | 1.00 | 47.16 | 6 |
| ATOM | 3731 | CZ | TYR | C | 26 | 44.227 | 102.384 | 5.282 | 1.00 | 46.35 | 6 |
| ATOM | 3732 | OH | TYR | C | 26 | 45.303 | 101.734 | 4.718 | 1.00 | 46.49 | 8 |
| ATOM | 3733 | C | TYR | C | 26 | 40.480 | 106.572 | 5.814 | 1.00 | 55.37 | 6 |
| ATOM | 3734 | O | TYR | C | 26 | 41.221 | 107.148 | 5.014 | 1.00 | 55.48 | 8 |
| ATOM | 3735 | N | LYS | C | 27 | 39.167 | 106.474 | 5.654 | 1.00 | 60.13 | 7 |
| ATOM | 3736 | CA | LYS | C | 27 | 38.474 | 107.031 | 4.503 | 1.00 | 60.39 | 6 |
| ATOM | 3737 | CB | LYS | C | 27 | 36.976 | 107.042 | 4.777 | 1.00 | 79.68 | 6 |
| ATOM | 3738 | CG | LYS | C | 27 | 36.140 | 107.634 | 3.670 | 1.00 | 81.33 | 6 |
| ATOM | 3739 | CD | LYS | C | 27 | 34.664 | 107.567 | 4.037 | 1.00 | 83.89 | 6 |
| ATOM | 3740 | CE | LYS | C | 27 | 33.765 | 108.050 | 2.909 | 1.00 | 85.68 | 6 |
| ATOM | 3741 | NZ | LYS | C | 27 | 32.338 | 107.749 | 3.215 | 1.00 | 86.18 | 7 |
| ATOM | 3742 | C | LYS | C | 27 | 38.947 | 108.442 | 4.188 | 1.00 | 60.15 | 6 |
| ATOM | 3743 | O | LYS | C | 27 | 39.368 | 108.729 | 3.070 | 1.00 | 60.17 | 8 |
| ATOM | 3744 | N | LYS | C | 28 | 38.889 | 109.317 | 5.188 | 1.00 | 50.23 | 7 |
| ATOM | 3745 | CA | LYS | C | 28 | 39.283 | 110.709 | 5.008 | 1.00 | 51.29 | 6 |
| ATOM | 3746 | CB | LYS | C | 28 | 38.838 | 111.557 | 6.205 | 1.00 | 208.87 | 6 |
| ATOM | 3747 | CG | LYS | C | 28 | 39.587 | 111.276 | 7.499 | 1.00 | 208.87 | 6 |
| ATOM | 3748 | CD | LYS | C | 28 | 39.211 | 112.278 | 8.581 | 1.00 | 208.87 | 6 |
| ATOM | 3749 | CE | LYS | C | 28 | 40.027 | 112.062 | 9.843 | 1.00 | 208.87 | 6 |
| ATOM | 3750 | NZ | LYS | C | 28 | 39.705 | 113.078 | 10.880 | 1.00 | 208.87 | 7 |
| ATOM | 3751 | C | LYS | C | 28 | 40.776 | 110.872 | 4.802 | 1.00 | 50.07 | 6 |
| ATOM | 3752 | O | LYS | C | 28 | 41.266 | 111.991 | 4.625 | 1.00 | 49.02 | 8 |
| ATOM | 3753 | N | ALA | C | 29 | 41.491 | 109.753 | 4.841 | 1.00 | 30.72 | 7 |
| ATOM | 3754 | CA | ALA | C | 29 | 42.934 | 109.748 | 4.647 | 1.00 | 30.33 | 6 |
| ATOM | 3755 | CB | ALA | C | 29 | 43.617 | 108.995 | 5.766 | 1.00 | 73.05 | 6 |
| ATOM | 3756 | C | ALA | C | 29 | 43.108 | 109.015 | 3.349 | 1.00 | 30.18 | 6 |
| ATOM | 3757 | O | ALA | C | 29 | 43.954 | 109.363 | 2.528 | 1.00 | 29.71 | 8 |
| ATOM | 3758 | N | LEU | C | 30 | 42.282 | 107.990 | 3.184 | 1.00 | 47.18 | 7 |
| ATOM | 3759 | CA | LEU | C | 30 | 42.264 | 107.170 | 1.988 | 1.00 | 49.80 | 6 |
| ATOM | 3760 | CB | LEU | C | 30 | 41.920 | 105.727 | 2.355 | 1.00 | 77.57 | 6 |
| ATOM | 3761 | CG | LEU | C | 30 | 41.923 | 104.720 | 1.207 | 1.00 | 79.55 | 6 |
| ATOM | 3762 | CD1 | LEU | C | 30 | 43.315 | 104.599 | 0.633 | 1.00 | 80.89 | 6 |
| ATOM | 3763 | CD2 | LEU | C | 30 | 41.438 | 103.384 | 1.708 | 1.00 | 79.00 | 6 |
| ATOM | 3764 | C | LEU | C | 30 | 41.190 | 107.770 | 1.067 | 1.00 | 50.91 | 6 |
| ATOM | 3765 | O | LEU | C | 30 | 40.442 | 107.056 | 0.390 | 1.00 | 50.20 | 8 |
| ATOM | 3766 | N | GLN | C | 31 | 41.126 | 109.100 | 1.071 | 1.00 | 108.17 | 7 |
| ATOM | 3767 | CA | GLN | C | 31 | 40.181 | 109.874 | 0.269 | 1.00 | 108.50 | 6 |
| ATOM | 3768 | CB | GLN | C | 31 | 40.839 | 111.190 | −0.137 | 1.00 | 55.88 | 6 |
| ATOM | 3769 | CG | GLN | C | 31 | 40.877 | 112.184 | 0.993 | 1.00 | 54.19 | 6 |
| ATOM | 3770 | CD | GLN | C | 31 | 41.888 | 113.291 | 0.797 | 1.00 | 54.53 | 6 |
| ATOM | 3771 | OE1 | GLN | C | 31 | 42.243 | 113.639 | −0.321 | 1.00 | 53.64 | 8 |
| ATOM | 3772 | NE2 | GLN | C | 31 | 42.350 | 113.864 | 1.900 | 1.00 | 55.77 | 7 |
| ATOM | 3773 | C | GLN | C | 31 | 39.666 | 109.141 | −0.963 | 1.00 | 110.03 | 6 |
| ATOM | 3774 | O | GLN | C | 31 | 38.491 | 109.245 | −1.312 | 1.00 | 110.78 | 8 |
| ATOM | 3775 | N | ALA | C | 32 | 40.567 | 108.412 | −1.610 | 1.00 | 112.39 | 7 |
| ATOM | 3776 | CA | ALA | C | 32 | 40.277 | 107.621 | −2.801 | 1.00 | 114.14 | 6 |
| ATOM | 3777 | CB | ALA | C | 32 | 40.562 | 106.153 | −2.511 | 1.00 | 162.72 | 6 |
| ATOM | 3778 | C | ALA | C | 32 | 38.888 | 107.755 | −3.409 | 1.00 | 115.03 | 6 |
| ATOM | 3779 | O | ALA | C | 32 | 38.744 | 108.151 | −4.563 | 1.00 | 115.39 | 8 |
| ATOM | 3780 | N | ASP | C | 33 | 37.873 | 107.405 | −2.630 | 1.00 | 75.66 | 7 |
| ATOM | 3781 | CA | ASP | C | 33 | 36.489 | 107.449 | −3.075 | 1.00 | 76.40 | 6 |
| ATOM | 3782 | CB | ASP | C | 33 | 35.594 | 106.892 | −1.967 | 1.00 | 98.63 | 6 |
| ATOM | 3783 | CG | ASP | C | 33 | 34.129 | 106.919 | −2.333 | 1.00 | 99.96 | 6 |
| ATOM | 3784 | OD1 | ASP | C | 33 | 33.366 | 107.650 | −1.662 | 1.00 | 100.46 | 8 |
| ATOM | 3785 | OD2 | ASP | C | 33 | 33.745 | 106.219 | −3.293 | 1.00 | 100.94 | 8 |
| ATOM | 3786 | C | ASP | C | 33 | 35.943 | 108.802 | −3.546 | 1.00 | 76.86 | 6 |
| ATOM | 3787 | O | ASP | C | 33 | 34.732 | 108.996 | −3.594 | 1.00 | 77.28 | 8 |
| ATOM | 3788 | N | VAL | C | 34 | 36.819 | 109.738 | −3.898 | 1.00 | 106.73 | 7 |
| ATOM | 3789 | CA | VAL | C | 34 | 36.361 | 111.039 | −4.392 | 1.00 | 108.50 | 6 |
| ATOM | 3790 | CB | VAL | C | 34 | 36.564 | 112.136 | −3.340 | 1.00 | 44.84 | 6 |
| ATOM | 3791 | CG1 | VAL | C | 34 | 37.139 | 113.387 | −3.962 | 1.00 | 43.21 | 6 |
| ATOM | 3792 | CG2 | VAL | C | 34 | 35.234 | 112.468 | −2.722 | 1.00 | 46.76 | 6 |
| ATOM | 3793 | C | VAL | C | 34 | 37.035 | 111.450 | −5.702 | 1.00 | 110.20 | 6 |
| ATOM | 3794 | O | VAL | C | 34 | 38.263 | 111.561 | −5.772 | 1.00 | 111.68 | 8 |
| ATOM | 3795 | N | PRO | C | 35 | 36.229 | 111.701 | −6.754 | 1.00 | 66.15 | 7 |
| ATOM | 3796 | CD | PRO | C | 35 | 34.774 | 111.914 | −6.644 | 1.00 | 152.93 | 6 |
| ATOM | 3797 | CA | PRO | C | 35 | 36.704 | 112.097 | −8.082 | 1.00 | 66.89 | 6 |
| ATOM | 3798 | CB | PRO | C | 35 | 35.556 | 112.940 | −8.609 | 1.00 | 152.71 | 6 |
| ATOM | 3799 | CG | PRO | C | 35 | 34.376 | 112.198 | −8.084 | 1.00 | 153.29 | 6 |
| ATOM | 3800 | C | PRO | C | 35 | 38.005 | 112.865 | −7.999 | 1.00 | 67.29 | 6 |
| ATOM | 3801 | O | PRO | C | 35 | 38.047 | 113.955 | −7.444 | 1.00 | 68.02 | 8 |
| ATOM | 3802 | N | PRO | C | 36 | 39.090 | 112.284 | −8.534 | 1.00 | 59.92 | 7 |
| ATOM | 3803 | CD | PRO | C | 36 | 39.041 | 110.962 | −9.189 | 1.00 | 60.17 | 6 |
| ATOM | 3804 | CA | PRO | C | 36 | 40.452 | 112.821 | −8.576 | 1.00 | 61.78 | 6 |
| ATOM | 3805 | CB | PRO | C | 36 | 41.056 | 112.088 | −9.763 | 1.00 | 60.22 | 6 |
| ATOM | 3806 | CG | PRO | C | 36 | 40.506 | 110.707 | −9.559 | 1.00 | 60.65 | 6 |
| ATOM | 3807 | C | PRO | C | 36 | 40.592 | 114.331 | −8.683 | 1.00 | 62.61 | 6 |
| ATOM | 3808 | O | PRO | C | 36 | 41.698 | 114.859 | −8.596 | 1.00 | 63.80 | 8 |
| ATOM | 3809 | N | GLU | C | 37 | 39.476 | 115.023 | −8.877 | 1.00 | 97.57 | 7 |
| ATOM | 3810 | CA | GLU | C | 37 | 39.491 | 116.475 | −8.975 | 1.00 | 98.61 | 6 |
| ATOM | 3811 | CB | GLU | C | 37 | 38.189 | 116.969 | −9.615 | 1.00 | 120.72 | 6 |
| ATOM | 3812 | CG | GLU | C | 37 | 37.946 | 116.409 | −11.018 | 1.00 | 122.45 | 6 |
| ATOM | 3813 | CD | GLU | C | 37 | 36.725 | 117.014 | −11.695 | 1.00 | 123.66 | 6 |
| ATOM | 3814 | OE1 | GLU | C | 37 | 36.713 | 118.245 | −11.915 | 1.00 | 124.43 | 8 |
| ATOM | 3815 | OE2 | GLU | C | 37 | 35.775 | 116.265 | −12.011 | 1.00 | 124.87 | 8 |
| ATOM | 3816 | C | GLU | C | 37 | 39.691 | 117.112 | −7.588 | 1.00 | 98.50 | 6 |
| ATOM | 3817 | O | GLU | C | 37 | 40.606 | 117.923 | −7.401 | 1.00 | 98.69 | 8 |
| ATOM | 3818 | N | LYS | C | 38 | 38.856 | 116.740 | −6.614 | 1.00 | 57.30 | 7 |
| ATOM | 3819 | CA | LYS | C | 38 | 38.989 | 117.296 | −5.265 | 1.00 | 57.57 | 6 |
| ATOM | 3820 | CB | LYS | C | 38 | 37.606 | 117.569 | −4.649 | 1.00 | 126.96 | 6 |
| ATOM | 3821 | CG | LYS | C | 38 | 36.600 | 116.430 | −4.700 | 1.00 | 128.66 | 6 |
| ATOM | 3822 | CD | LYS | C | 38 | 35.284 | 116.855 | −4.040 | 1.00 | 129.19 | 6 |
| ATOM | 3823 | CE | LYS | C | 38 | 34.243 | 115.747 | −4.058 | 1.00 | 129.10 | 6 |
| ATOM | 3824 | NZ | LYS | C | 38 | 32.995 | 116.155 | −3.356 | 1.00 | 129.49 | 7 |
| ATOM | 3825 | C | LYS | C | 38 | 39.859 | 116.493 | −4.284 | 1.00 | 57.41 | 6 |
| ATOM | 3826 | O | LYS | C | 38 | 40.545 | 115.538 | −4.665 | 1.00 | 55.85 | 8 |
| ATOM | 3827 | N | ARG | C | 39 | 39.819 | 116.895 | −3.016 | 1.00 | 79.57 | 7 |
| ATOM | 3828 | CA | ARG | C | 39 | 40.634 | 116.273 | −1.977 | 1.00 | 80.58 | 6 |
| ATOM | 3829 | CB | ARG | C | 39 | 40.572 | 114.748 | −2.073 | 1.00 | 131.15 | 6 |
| ATOM | 3830 | CG | ARG | C | 39 | 39.238 | 114.116 | −1.677 | 1.00 | 133.62 | 6 |
| ATOM | 3831 | CD | ARG | C | 39 | 38.881 | 114.351 | −0.209 | 1.00 | 136.29 | 6 |
| ATOM | 3832 | NE | ARG | C | 39 | 37.995 | 113.303 | 0.304 | 1.00 | 139.40 | 7 |
| ATOM | 3833 | CZ | ARG | C | 39 | 37.444 | 113.300 | 1.516 | 1.00 | 140.64 | 6 |
| ATOM | 3834 | NH1 | ARG | C | 39 | 37.675 | 114.296 | 2.363 | 1.00 | 141.73 | 7 |
| ATOM | 3835 | NH2 | ARG | C | 39 | 36.664 | 112.294 | 1.889 | 1.00 | 141.12 | 7 |
| ATOM | 3836 | C | ARG | C | 39 | 42.039 | 116.774 | −2.289 | 1.00 | 80.41 | 6 |
| ATOM | 3837 | O | ARG | C | 39 | 42.501 | 116.635 | −3.422 | 1.00 | 80.59 | 8 |
| ATOM | 3838 | N | GLU | C | 40 | 42.714 | 117.368 | −1.306 | 1.00 | 133.71 | 7 |
| ATOM | 3839 | CA | GLU | C | 40 | 44.045 | 117.921 | −1.554 | 1.00 | 134.19 | 6 |
| ATOM | 3840 | CB | GLU | C | 40 | 44.014 | 119.450 | −1.429 | 1.00 | 153.39 | 6 |
| ATOM | 3841 | CG | GLU | C | 40 | 45.378 | 120.116 | −1.608 | 1.00 | 154.96 | 6 |
| ATOM | 3842 | CD | GLU | C | 40 | 45.289 | 121.626 | −1.736 | 1.00 | 155.83 | 6 |
| ATOM | 3843 | OE1 | GLU | C | 40 | 44.748 | 122.276 | −0.820 | 1.00 | 156.11 | 8 |
| ATOM | 3844 | OE2 | GLU | C | 40 | 45.765 | 122.162 | −2.757 | 1.00 | 156.29 | 8 |
| ATOM | 3845 | C | GLU | C | 40 | 45.210 | 117.396 | −0.731 | 1.00 | 133.67 | 6 |
| ATOM | 3846 | O | GLU | C | 40 | 46.237 | 117.014 | −1.292 | 1.00 | 133.47 | 8 |
| ATOM | 3847 | N | ASN | C | 41 | 45.079 | 117.393 | 0.591 | 1.00 | 48.89 | 7 |
| ATOM | 3848 | CA | ASN | C | 41 | 46.184 | 116.913 | 1.410 | 1.00 | 48.90 | 6 |
| ATOM | 3849 | CB | ASN | C | 41 | 46.763 | 118.069 | 2.228 | 1.00 | 130.74 | 6 |
| ATOM | 3850 | CG | ASN | C | 41 | 47.479 | 119.084 | 1.364 | 1.00 | 132.51 | 6 |
| ATOM | 3851 | OD1 | ASN | C | 41 | 48.284 | 118.723 | 0.502 | 1.00 | 133.26 | 8 |
| ATOM | 3852 | ND2 | ASN | C | 41 | 47.199 | 120.362 | 1.593 | 1.00 | 133.67 | 7 |
| ATOM | 3853 | C | ASN | C | 41 | 45.946 | 115.713 | 2.328 | 1.00 | 47.57 | 6 |
| ATOM | 3854 | O | ASN | C | 41 | 44.923 | 115.032 | 2.263 | 1.00 | 47.19 | 8 |
| ATOM | 3855 | N | VAL | C | 42 | 46.941 | 115.457 | 3.168 | 1.00 | 113.68 | 7 |
| ATOM | 3856 | CA | VAL | C | 42 | 46.908 | 114.361 | 4.123 | 1.00 | 113.62 | 6 |
| ATOM | 3857 | CB | VAL | C | 42 | 45.718 | 114.516 | 5.104 | 1.00 | 120.00 | 6 |
| ATOM | 3858 | CG1 | VAL | C | 42 | 45.857 | 113.533 | 6.264 | 1.00 | 119.44 | 6 |
| ATOM | 3859 | CG2 | VAL | C | 42 | 45.661 | 115.946 | 5.620 | 1.00 | 120.00 | 6 |
| ATOM | 3860 | C | VAL | C | 42 | 46.834 | 112.990 | 3.443 | 1.00 | 113.04 | 6 |
| ATOM | 3861 | O | VAL | C | 42 | 46.163 | 112.811 | 2.418 | 1.00 | 112.64 | 8 |
| ATOM | 3862 | N | GLY | C | 43 | 47.554 | 112.037 | 4.028 | 1.00 | 97.34 | 7 |
| ATOM | 3863 | CA | GLY | C | 43 | 47.580 | 110.680 | 3.526 | 1.00 | 95.90 | 6 |
| ATOM | 3864 | C | GLY | C | 43 | 47.760 | 110.517 | 2.033 | 1.00 | 95.43 | 6 |
| ATOM | 3865 | O | GLY | C | 43 | 48.371 | 111.348 | 1.364 | 1.00 | 95.25 | 8 |
| ATOM | 3866 | N | ILE | C | 44 | 47.216 | 109.411 | 1.532 | 1.00 | 98.61 | 7 |
| ATOM | 3867 | CA | ILE | C | 44 | 47.267 | 109.030 | 0.126 | 1.00 | 97.86 | 6 |
| ATOM | 3868 | CB | ILE | C | 44 | 45.845 | 108.599 | −0.360 | 1.00 | 117.32 | 6 |
| ATOM | 3869 | CG2 | ILE | C | 44 | 45.725 | 108.710 | −1.883 | 1.00 | 118.51 | 6 |
| ATOM | 3870 | CG1 | ILE | C | 44 | 45.567 | 107.156 | 0.076 | 1.00 | 117.91 | 6 |
| ATOM | 3871 | CD1 | ILE | C | 44 | 45.689 | 106.909 | 1.559 | 1.00 | 119.46 | 6 |
| ATOM | 3872 | C | ILE | C | 44 | 47.842 | 110.086 | −0.815 | 1.00 | 96.77 | 6 |
| ATOM | 3873 | O | ILE | C | 44 | 48.942 | 109.927 | −1.344 | 1.00 | 96.62 | 8 |
| ATOM | 3874 | N | GLN | C | 45 | 47.098 | 111.168 | −1.001 | 1.00 | 64.52 | 7 |
| ATOM | 3875 | CA | GLN | C | 45 | 47.503 | 112.235 | −1.899 | 1.00 | 63.24 | 6 |
| ATOM | 3876 | CB | GLN | C | 45 | 46.306 | 113.144 | −2.178 | 1.00 | 134.09 | 6 |
| ATOM | 3877 | CG | GLN | C | 45 | 46.562 | 114.199 | −3.226 | 1.00 | 134.29 | 6 |
| ATOM | 3878 | CD | GLN | C | 45 | 45.286 | 114.852 | −3.692 | 1.00 | 135.13 | 6 |
| ATOM | 3879 | OE1 | GLN | C | 45 | 44.484 | 115.316 | −2.883 | 1.00 | 136.75 | 8 |
| ATOM | 3880 | NE2 | GLN | C | 45 | 45.090 | 114.896 | −5.003 | 1.00 | 135.00 | 7 |
| ATOM | 3881 | C | GLN | C | 45 | 48.677 | 113.053 | −1.386 | 1.00 | 61.15 | 6 |
| ATOM | 3882 | O | GLN | C | 45 | 49.646 | 113.280 | −2.110 | 1.00 | 60.47 | 8 |
| ATOM | 3883 | N | ALA | C | 46 | 48.585 | 113.510 | −0.143 | 1.00 | 62.10 | 7 |
| ATOM | 3884 | CA | ALA | C | 46 | 49.661 | 114.294 | 0.445 | 1.00 | 60.83 | 6 |
| ATOM | 3885 | CB | ALA | C | 46 | 49.509 | 114.356 | 1.955 | 1.00 | 131.32 | 6 |
| ATOM | 3886 | C | ALA | C | 46 | 50.954 | 113.594 | 0.080 | 1.00 | 59.10 | 6 |
| ATOM | 3887 | O | ALA | C | 46 | 51.696 | 114.060 | −0.776 | 1.00 | 58.97 | 8 |
| ATOM | 3888 | N | ALA | C | 47 | 51.191 | 112.452 | 0.716 | 1.00 | 76.48 | 7 |
| ATOM | 3889 | CA | ALA | C | 47 | 52.387 | 111.657 | 0.467 | 1.00 | 75.00 | 6 |
| ATOM | 3890 | CB | ALA | C | 47 | 52.158 | 110.221 | 0.947 | 1.00 | 76.20 | 6 |
| ATOM | 3891 | C | ALA | C | 47 | 52.780 | 111.674 | −1.019 | 1.00 | 74.00 | 6 |
| ATOM | 3892 | O | ALA | C | 47 | 53.961 | 111.600 | −1.368 | 1.00 | 74.34 | 8 |
| ATOM | 3893 | N | PHE | C | 48 | 51.787 | 111.767 | −1.895 | 1.00 | 40.16 | 7 |
| ATOM | 3894 | CA | PHE | C | 48 | 52.065 | 111.811 | −3.313 | 1.00 | 38.41 | 6 |
| ATOM | 3895 | CB | PHE | C | 48 | 50.795 | 111.553 | −4.122 | 1.00 | 63.46 | 6 |
| ATOM | 3896 | CG | PHE | C | 48 | 50.569 | 110.105 | −4.456 | 1.00 | 63.24 | 6 |
| ATOM | 3897 | CD1 | PHE | C | 48 | 51.206 | 109.099 | −3.735 | 1.00 | 62.54 | 6 |
| ATOM | 3898 | CD2 | PHE | C | 48 | 49.701 | 109.747 | −5.481 | 1.00 | 63.01 | 6 |
| ATOM | 3899 | CE1 | PHE | C | 48 | 50.983 | 107.766 | −4.031 | 1.00 | 61.74 | 6 |
| ATOM | 3900 | CE2 | PHE | C | 48 | 49.470 | 108.414 | −5.780 | 1.00 | 61.69 | 6 |
| ATOM | 3901 | CZ | PHE | C | 48 | 50.111 | 107.422 | −5.056 | 1.00 | 61.01 | 6 |
| ATOM | 3902 | C | PHE | C | 48 | 52.612 | 113.185 | −3.618 | 1.00 | 38.13 | 6 |
| ATOM | 3903 | O | PHE | C | 48 | 53.794 | 113.320 | −3.942 | 1.00 | 39.04 | 8 |
| ATOM | 3904 | N | LYS | C | 49 | 51.756 | 114.202 | −3.500 | 1.00 | 31.04 | 7 |
| ATOM | 3905 | CA | LYS | C | 49 | 52.164 | 115.574 | −3.760 | 1.00 | 29.55 | 6 |
| ATOM | 3906 | CB | LYS | C | 49 | 51.042 | 116.541 | −3.420 | 1.00 | 43.45 | 6 |
| ATOM | 3907 | CG | LYS | C | 49 | 49.942 | 116.566 | −4.457 | 1.00 | 43.78 | 6 |
| ATOM | 3908 | CD | LYS | C | 49 | 48.890 | 117.607 | −4.106 | 1.00 | 44.92 | 6 |
| ATOM | 3909 | CE | LYS | C | 49 | 47.993 | 117.952 | −5.295 | 1.00 | 45.14 | 6 |
| ATOM | 3910 | NZ | LYS | C | 49 | 47.157 | 119.174 | −5.031 | 1.00 | 45.76 | 7 |
| ATOM | 3911 | C | LYS | C | 49 | 53.383 | 115.848 | −2.912 | 1.00 | 29.35 | 6 |
| ATOM | 3912 | O | LYS | C | 49 | 54.371 | 116.390 | −3.391 | 1.00 | 28.88 | 8 |
| ATOM | 3913 | N | GLU | C | 50 | 53.325 | 115.451 | −1.652 | 1.00 | 18.42 | 7 |
| ATOM | 3914 | CA | GLU | C | 50 | 54.461 | 115.636 | −0.768 | 1.00 | 20.92 | 6 |
| ATOM | 3915 | CB | GLU | C | 50 | 54.285 | 114.825 | 0.515 | 1.00 | 124.61 | 6 |
| ATOM | 3916 | CG | GLU | C | 50 | 53.448 | 115.494 | 1.583 | 1.00 | 127.58 | 6 |
| ATOM | 3917 | CD | GLU | C | 50 | 53.419 | 114.679 | 2.851 | 1.00 | 129.37 | 6 |
| ATOM | 3918 | OE1 | GLU | C | 50 | 54.507 | 114.257 | 3.299 | 1.00 | 128.96 | 8 |
| ATOM | 3919 | OE2 | GLU | C | 50 | 52.315 | 114.463 | 3.397 | 1.00 | 132.46 | 8 |
| ATOM | 3920 | C | GLU | C | 50 | 55.743 | 115.194 | −1.467 | 1.00 | 21.08 | 6 |
| ATOM | 3921 | O | GLU | C | 50 | 56.631 | 116.000 | −1.719 | 1.00 | 22.13 | 8 |
| ATOM | 3922 | N | THR | C | 51 | 55.852 | 113.911 | −1.777 | 1.00 | 71.11 | 7 |
| ATOM | 3923 | CA | THR | C | 51 | 57.046 | 113.427 | −2.442 | 1.00 | 72.37 | 6 |
| ATOM | 3924 | CB | THR | C | 51 | 56.886 | 111.980 | −2.832 | 1.00 | 31.71 | 6 |
| ATOM | 3925 | OG1 | THR | C | 51 | 56.273 | 111.262 | −1.759 | 1.00 | 30.28 | 8 |
| ATOM | 3926 | CG2 | THR | C | 51 | 58.230 | 111.386 | −3.103 | 1.00 | 31.58 | 6 |
| ATOM | 3927 | C | THR | C | 51 | 57.249 | 114.262 | −3.697 | 1.00 | 74.48 | 6 |
| ATOM | 3928 | O | THR | C | 51 | 56.557 | 114.071 | −4.701 | 1.00 | 74.18 | 8 |
| ATOM | 3929 | N | PHE | C | 52 | 58.194 | 115.195 | −3.641 | 1.00 | 207.23 | 7 |
| ATOM | 3930 | CA | PHE | C | 52 | 58.437 | 116.065 | −4.781 | 1.00 | 208.87 | 6 |
| ATOM | 3931 | CB | PHE | C | 52 | 59.544 | 117.102 | −4.471 | 1.00 | 106.21 | 6 |
| ATOM | 3932 | CG | PHE | C | 52 | 60.822 | 116.525 | −3.893 | 1.00 | 106.76 | 6 |
| ATOM | 3933 | CD1 | PHE | C | 52 | 61.419 | 115.387 | −4.434 | 1.00 | 106.34 | 6 |
| ATOM | 3934 | CD2 | PHE | C | 52 | 61.462 | 117.171 | −2.836 | 1.00 | 106.63 | 6 |
| ATOM | 3935 | CE1 | PHE | C | 52 | 62.635 | 114.907 | −3.930 | 1.00 | 106.88 | 6 |
| ATOM | 3936 | CE2 | PHE | C | 52 | 62.675 | 116.701 | −2.327 | 1.00 | 106.95 | 6 |
| ATOM | 3937 | CZ | PHE | C | 52 | 63.262 | 115.568 | −2.874 | 1.00 | 107.20 | 6 |
| ATOM | 3938 | C | PHE | C | 52 | 58.752 | 115.329 | −6.079 | 1.00 | 208.87 | 6 |
| ATOM | 3939 | O | PHE | C | 52 | 59.429 | 114.299 | −6.075 | 1.00 | 208.87 | 8 |
| ATOM | 3940 | N | PRO | C | 53 | 58.227 | 115.837 | −7.209 | 1.00 | 122.42 | 7 |
| ATOM | 3941 | CD | PRO | C | 53 | 57.338 | 117.005 | −7.345 | 1.00 | 39.07 | 6 |
| ATOM | 3942 | CA | PRO | C | 53 | 58.467 | 115.224 | −8.514 | 1.00 | 122.58 | 6 |
| ATOM | 3943 | CB | PRO | C | 53 | 58.031 | 116.313 | −9.478 | 1.00 | 38.23 | 6 |
| ATOM | 3944 | CG | PRO | C | 53 | 56.863 | 116.884 | −8.778 | 1.00 | 38.12 | 6 |
| ATOM | 3945 | C | PRO | C | 53 | 59.947 | 114.921 | −8.634 | 1.00 | 123.24 | 6 |
| ATOM | 3946 | O | PRO | C | 53 | 60.766 | 115.837 | −8.679 | 1.00 | 124.61 | 8 |
| ATOM | 3947 | N | ILE | C | 54 | 60.293 | 113.641 | −8.676 | 1.00 | 59.25 | 7 |
| ATOM | 3948 | CA | ILE | C | 54 | 61.691 | 113.260 | −8.772 | 1.00 | 59.00 | 6 |
| ATOM | 3949 | CB | ILE | C | 54 | 61.854 | 111.761 | −9.153 | 1.00 | 38.24 | 6 |
| ATOM | 3950 | CG2 | ILE | C | 54 | 60.999 | 111.441 | −10.361 | 1.00 | 38.42 | 6 |
| ATOM | 3951 | CG1 | ILE | C | 54 | 63.335 | 111.442 | −9.418 | 1.00 | 38.13 | 6 |
| ATOM | 3952 | CD1 | ILE | C | 54 | 64.270 | 111.795 | −8.272 | 1.00 | 35.57 | 6 |
| ATOM | 3953 | C | ILE | C | 54 | 62.472 | 114.127 | −9.753 | 1.00 | 59.79 | 6 |
| ATOM | 3954 | O | ILE | C | 54 | 62.206 | 114.152 | −10.955 | 1.00 | 59.16 | 8 |
| ATOM | 3955 | N | GLU | C | 55 | 63.420 | 114.871 | −9.199 | 1.00 | 61.34 | 7 |
| ATOM | 3956 | CA | GLU | C | 55 | 64.285 | 115.720 | −9.988 | 1.00 | 63.19 | 6 |
| ATOM | 3957 | CB | GLU | C | 55 | 65.047 | 116.698 | −9.085 | 1.00 | 88.10 | 6 |
| ATOM | 3958 | CG | GLU | C | 55 | 64.181 | 117.597 | −8.191 | 1.00 | 89.00 | 6 |
| ATOM | 3959 | CD | GLU | C | 55 | 63.485 | 116.848 | −7.068 | 1.00 | 89.60 | 6 |
| ATOM | 3960 | OE1 | GLU | C | 55 | 64.139 | 116.016 | −6.408 | 1.00 | 90.86 | 8 |
| ATOM | 3961 | OE2 | GLU | C | 55 | 62.288 | 117.106 | −6.833 | 1.00 | 88.74 | 8 |
| ATOM | 3962 | C | GLU | C | 55 | 65.256 | 114.720 | −10.603 | 1.00 | 64.85 | 6 |
| ATOM | 3963 | O | GLU | C | 55 | 64.879 | 113.577 | −10.856 | 1.00 | 65.18 | 8 |
| ATOM | 3964 | N | GLU | C | 56 | 66.501 | 115.128 | −10.824 | 1.00 | 154.58 | 7 |
| ATOM | 3965 | CA | GLU | C | 56 | 67.494 | 114.226 | −11.397 | 1.00 | 157.05 | 6 |
| ATOM | 3966 | CB | GLU | C | 56 | 67.211 | 113.985 | −12.881 | 1.00 | 124.00 | 6 |
| ATOM | 3967 | CG | GLU | C | 56 | 67.245 | 115.244 | −13.718 | 1.00 | 125.79 | 6 |
| ATOM | 3968 | CD | GLU | C | 56 | 66.159 | 116.225 | −13.323 | 1.00 | 127.18 | 6 |
| ATOM | 3969 | OE1 | GLU | C | 56 | 64.979 | 115.979 | −13.655 | 1.00 | 127.61 | 8 |
| ATOM | 3970 | OE2 | GLU | C | 56 | 66.484 | 117.239 | −12.667 | 1.00 | 127.56 | 8 |
| ATOM | 3971 | C | GLU | C | 56 | 68.891 | 114.795 | −11.240 | 1.00 | 158.22 | 6 |
| ATOM | 3972 | O | GLU | C | 56 | 69.172 | 115.529 | −10.294 | 1.00 | 158.76 | 8 |
| ATOM | 3973 | N | GLY | C | 57 | 69.765 | 114.445 | −12.176 | 1.00 | 103.07 | 7 |
| ATOM | 3974 | CA | GLY | C | 57 | 71.129 | 114.932 | −12.130 | 1.00 | 104.55 | 6 |
| ATOM | 3975 | C | GLY | C | 57 | 71.238 | 116.354 | −12.641 | 1.00 | 105.62 | 6 |
| ATOM | 3976 | O | GLY | C | 57 | 70.732 | 116.685 | −13.720 | 1.00 | 106.41 | 8 |
| ATOM | 3977 | N | ALA | C | 58 | 71.898 | 117.202 | −11.860 | 1.00 | 132.92 | 7 |
| ATOM | 3978 | CA | ALA | C | 58 | 72.075 | 118.592 | −12.240 | 1.00 | 133.77 | 6 |
| ATOM | 3979 | CB | ALA | C | 58 | 72.757 | 118.657 | −13.590 | 1.00 | 19.77 | 6 |
| ATOM | 3980 | C | ALA | C | 58 | 70.722 | 119.303 | −12.289 | 1.00 | 134.75 | 6 |
| ATOM | 3981 | O | ALA | C | 58 | 69.912 | 119.053 | −13.187 | 1.00 | 134.98 | 8 |
| ATOM | 3982 | N | LYS | C | 59 | 70.492 | 120.188 | −11.318 | 1.00 | 156.20 | 7 |
| ATOM | 3983 | CA | LYS | C | 59 | 69.246 | 120.950 | −11.200 | 1.00 | 157.09 | 6 |
| ATOM | 3984 | CB | LYS | C | 59 | 69.438 | 122.137 | −10.246 | 1.00 | 107.35 | 6 |
| ATOM | 3985 | CG | LYS | C | 59 | 69.663 | 121.752 | −8.784 | 1.00 | 107.22 | 6 |
| ATOM | 3986 | CD | LYS | C | 59 | 69.714 | 122.989 | −7.877 | 1.00 | 106.92 | 6 |
| ATOM | 3987 | CE | LYS | C | 59 | 69.795 | 122.617 | −6.393 | 1.00 | 106.52 | 6 |
| ATOM | 3988 | NZ | LYS | C | 59 | 69.818 | 123.818 | −5.509 | 1.00 | 105.42 | 7 |
| ATOM | 3989 | C | LYS | C | 59 | 68.684 | 121.463 | −12.524 | 1.00 | 157.61 | 6 |
| ATOM | 3990 | O | LYS | C | 59 | 69.074 | 122.530 | −13.006 | 1.00 | 157.43 | 8 |
| ATOM | 3991 | N | GLY | C | 60 | 67.752 | 120.701 | −13.094 | 1.00 | 129.14 | 7 |
| ATOM | 3992 | CA | GLY | C | 60 | 67.137 | 121.081 | −14.353 | 1.00 | 129.03 | 6 |
| ATOM | 3993 | C | GLY | C | 60 | 68.148 | 121.434 | −15.429 | 1.00 | 129.06 | 6 |
| ATOM | 3994 | O | GLY | C | 60 | 68.306 | 122.606 | −15.779 | 1.00 | 129.14 | 8 |
| ATOM | 3995 | N | LYS | C | 61 | 68.832 | 120.419 | −15.952 | 1.00 | 189.07 | 7 |
| ATOM | 3996 | CA | LYS | C | 61 | 69.837 | 120.612 | −16.997 | 1.00 | 188.42 | 6 |
| ATOM | 3997 | CB | LYS | C | 61 | 71.193 | 120.067 | −16.531 | 1.00 | 95.41 | 6 |
| ATOM | 3998 | CG | LYS | C | 61 | 71.836 | 120.846 | −15.397 | 1.00 | 95.06 | 6 |
| ATOM | 3999 | CD | LYS | C | 61 | 72.555 | 122.091 | −15.882 | 1.00 | 94.98 | 6 |
| ATOM | 4000 | CE | LYS | C | 61 | 73.320 | 122.758 | −14.738 | 1.00 | 94.81 | 6 |
| ATOM | 4001 | NZ | LYS | C | 61 | 74.117 | 123.930 | −15.204 | 1.00 | 94.67 | 7 |
| ATOM | 4002 | C | LYS | C | 61 | 69.433 | 119.927 | −18.304 | 1.00 | 187.52 | 6 |
| ATOM | 4003 | O | LYS | C | 61 | 69.906 | 120.301 | −19.380 | 1.00 | 187.21 | 8 |
| ATOM | 4004 | N | GLY | C | 62 | 68.561 | 118.927 | −18.207 | 1.00 | 173.41 | 7 |
| ATOM | 4005 | CA | GLY | C | 62 | 68.121 | 118.212 | −19.394 | 1.00 | 172.55 | 6 |
| ATOM | 4006 | C | GLY | C | 62 | 66.715 | 117.645 | −19.301 | 1.00 | 171.94 | 6 |
| ATOM | 4007 | O | GLY | C | 62 | 65.782 | 118.334 | −18.882 | 1.00 | 172.35 | 8 |
| ATOM | 4008 | N | GLY | C | 63 | 66.562 | 116.382 | −19.692 | 1.00 | 106.71 | 7 |
| ATOM | 4009 | CA | GLY | C | 63 | 65.255 | 115.752 | −19.651 | 1.00 | 104.35 | 6 |
| ATOM | 4010 | C | GLY | C | 63 | 65.002 | 114.888 | −18.430 | 1.00 | 102.47 | 6 |
| ATOM | 4011 | O | GLY | C | 63 | 65.893 | 114.685 | −17.604 | 1.00 | 102.37 | 8 |
| ATOM | 4012 | N | LEU | C | 64 | 63.769 | 114.394 | −18.330 | 1.00 | 153.39 | 7 |
| ATOM | 4013 | CA | LEU | C | 64 | 63.305 | 113.529 | −17.240 | 1.00 | 151.74 | 6 |
| ATOM | 4014 | CB | LEU | C | 64 | 64.362 | 112.473 | −16.911 | 1.00 | 130.45 | 6 |
| ATOM | 4015 | CG | LEU | C | 64 | 64.731 | 111.550 | −18.072 | 1.00 | 129.19 | 6 |
| ATOM | 4016 | CD1 | LEU | C | 64 | 65.827 | 110.609 | −17.627 | 1.00 | 128.90 | 6 |
| ATOM | 4017 | CD2 | LEU | C | 64 | 63.509 | 110.775 | −18.533 | 1.00 | 129.76 | 6 |
| ATOM | 4018 | C | LEU | C | 64 | 62.891 | 114.261 | −15.965 | 1.00 | 150.41 | 6 |
| ATOM | 4019 | O | LEU | C | 64 | 63.715 | 114.536 | −15.097 | 1.00 | 150.11 | 8 |
| ATOM | 4020 | N | VAL | C | 65 | 61.599 | 114.564 | −15.864 | 1.00 | 94.25 | 7 |
| ATOM | 4021 | CA | VAL | C | 65 | 61.043 | 115.254 | −14.703 | 1.00 | 93.03 | 6 |
| ATOM | 4022 | CB | VAL | C | 65 | 60.210 | 116.477 | −15.131 | 1.00 | 49.35 | 6 |
| ATOM | 4023 | CG1 | VAL | C | 65 | 59.347 | 116.972 | −13.990 | 1.00 | 48.78 | 6 |
| ATOM | 4024 | CG2 | VAL | C | 65 | 61.131 | 117.571 | −15.572 | 1.00 | 49.43 | 6 |
| ATOM | 4025 | C | VAL | C | 65 | 60.142 | 114.304 | −13.925 | 1.00 | 93.26 | 6 |
| ATOM | 4026 | O | VAL | C | 65 | 60.385 | 114.015 | −12.747 | 1.00 | 93.73 | 8 |
| ATOM | 4027 | N | LEU | C | 66 | 59.099 | 113.828 | −14.601 | 1.00 | 62.83 | 7 |
| ATOM | 4028 | CA | LEU | C | 66 | 58.137 | 112.902 | −14.017 | 1.00 | 60.91 | 6 |
| ATOM | 4029 | CB | LEU | C | 66 | 58.833 | 111.620 | −13.543 | 1.00 | 55.02 | 6 |
| ATOM | 4030 | CG | LEU | C | 66 | 57.939 | 110.554 | −12.900 | 1.00 | 54.34 | 6 |
| ATOM | 4031 | CD1 | LEU | C | 66 | 56.776 | 110.172 | −13.817 | 1.00 | 53.57 | 6 |
| ATOM | 4032 | CD2 | LEU | C | 66 | 58.798 | 109.350 | −12.578 | 1.00 | 54.77 | 6 |
| ATOM | 4033 | C | LEU | C | 66 | 57.368 | 113.504 | −12.867 | 1.00 | 59.76 | 6 |
| ATOM | 4034 | O | LEU | C | 66 | 57.903 | 113.717 | −11.780 | 1.00 | 58.86 | 8 |
| ATOM | 4035 | N | ASP | C | 67 | 56.101 | 113.786 | −13.125 | 1.00 | 83.71 | 7 |
| ATOM | 4036 | CA | ASP | C | 67 | 55.234 | 114.338 | −12.111 | 1.00 | 84.15 | 6 |
| ATOM | 4037 | CB | ASP | C | 67 | 54.619 | 115.662 | −12.588 | 1.00 | 162.72 | 6 |
| ATOM | 4038 | CG | ASP | C | 67 | 54.463 | 115.733 | −14.100 | 1.00 | 164.62 | 6 |
| ATOM | 4039 | OD1 | ASP | C | 67 | 55.465 | 115.526 | −14.817 | 1.00 | 164.61 | 8 |
| ATOM | 4040 | OD2 | ASP | C | 67 | 53.339 | 116.011 | −14.570 | 1.00 | 166.53 | 8 |
| ATOM | 4041 | C | ASP | C | 67 | 54.164 | 113.299 | −11.820 | 1.00 | 83.51 | 6 |
| ATOM | 4042 | O | ASP | C | 67 | 54.057 | 112.295 | −12.531 | 1.00 | 83.41 | 8 |
| ATOM | 4043 | N | PHE | C | 68 | 53.390 | 113.526 | −10.765 | 1.00 | 28.71 | 7 |
| ATOM | 4044 | CA | PHE | C | 68 | 52.343 | 112.596 | −10.385 | 1.00 | 28.07 | 6 |
| ATOM | 4045 | CB | PHE | C | 68 | 52.508 | 112.183 | −8.912 | 1.00 | 91.06 | 6 |
| ATOM | 4046 | CG | PHE | C | 68 | 53.892 | 111.617 | −8.571 | 1.00 | 92.16 | 6 |
| ATOM | 4047 | CD1 | PHE | C | 68 | 55.010 | 112.452 | −8.483 | 1.00 | 92.24 | 6 |
| ATOM | 4048 | CD2 | PHE | C | 68 | 54.069 | 110.251 | −8.321 | 1.00 | 91.63 | 6 |
| ATOM | 4049 | CE1 | PHE | C | 68 | 56.269 | 111.932 | −8.150 | 1.00 | 91.63 | 6 |
| ATOM | 4050 | CE2 | PHE | C | 68 | 55.327 | 109.731 | −7.989 | 1.00 | 90.24 | 6 |
| ATOM | 4051 | CZ | PHE | C | 68 | 56.422 | 110.571 | −7.904 | 1.00 | 90.55 | 6 |
| ATOM | 4052 | C | PHE | C | 68 | 50.983 | 113.236 | −10.621 | 1.00 | 27.88 | 6 |
| ATOM | 4053 | O | PHE | C | 68 | 50.844 | 114.454 | −10.543 | 1.00 | 26.24 | 8 |
| ATOM | 4054 | N | LEU | C | 69 | 49.993 | 112.405 | −10.941 | 1.00 | 131.88 | 7 |
| ATOM | 4055 | CA | LEU | C | 69 | 48.627 | 112.862 | −11.193 | 1.00 | 132.72 | 6 |
| ATOM | 4056 | CB | LEU | C | 69 | 48.188 | 112.515 | −12.617 | 1.00 | 63.15 | 6 |
| ATOM | 4057 | CG | LEU | C | 69 | 49.022 | 112.904 | −13.834 | 1.00 | 63.27 | 6 |
| ATOM | 4058 | CD1 | LEU | C | 69 | 48.220 | 112.557 | −15.096 | 1.00 | 62.57 | 6 |
| ATOM | 4059 | CD2 | LEU | C | 69 | 49.365 | 114.391 | −13.795 | 1.00 | 63.36 | 6 |
| ATOM | 4060 | C | LEU | C | 69 | 47.668 | 112.176 | −10.221 | 1.00 | 133.30 | 6 |
| ATOM | 4061 | O | LEU | C | 69 | 47.779 | 112.332 | −9.002 | 1.00 | 134.07 | 8 |
| ATOM | 4062 | N | GLU | C | 70 | 46.730 | 111.410 | −10.775 | 1.00 | 73.64 | 7 |
| ATOM | 4063 | CA | GLU | C | 70 | 45.758 | 110.691 | −9.966 | 1.00 | 74.13 | 6 |
| ATOM | 4064 | CB | GLU | C | 70 | 44.628 | 110.115 | −10.825 | 1.00 | 82.92 | 6 |
| ATOM | 4065 | CG | GLU | C | 70 | 44.808 | 110.252 | −12.315 | 1.00 | 84.69 | 6 |
| ATOM | 4066 | CD | GLU | C | 70 | 44.267 | 111.559 | −12.825 | 1.00 | 85.45 | 6 |
| ATOM | 4067 | OE1 | GLU | C | 70 | 44.775 | 112.616 | −12.402 | 1.00 | 85.54 | 8 |
| ATOM | 4068 | OE2 | GLU | C | 70 | 43.328 | 111.525 | −13.643 | 1.00 | 86.32 | 8 |
| ATOM | 4069 | C | GLU | C | 70 | 46.387 | 109.546 | −9.192 | 1.00 | 73.87 | 6 |
| ATOM | 4070 | O | GLU | C | 70 | 47.593 | 109.341 | −9.202 | 1.00 | 74.29 | 8 |
| ATOM | 4071 | N | TYR | C | 71 | 45.525 | 108.794 | −8.534 | 1.00 | 41.60 | 7 |
| ATOM | 4072 | CA | TYR | C | 71 | 45.908 | 107.657 | −7.720 | 1.00 | 41.39 | 6 |
| ATOM | 4073 | CB | TYR | C | 71 | 46.016 | 108.097 | −6.254 | 1.00 | 96.85 | 6 |
| ATOM | 4074 | CG | TYR | C | 71 | 44.711 | 108.649 | −5.736 | 1.00 | 99.37 | 6 |
| ATOM | 4075 | CD1 | TYR | C | 71 | 43.794 | 107.826 | −5.096 | 1.00 | 100.19 | 6 |
| ATOM | 4076 | CE1 | TYR | C | 71 | 42.526 | 108.288 | −4.753 | 1.00 | 101.02 | 6 |
| ATOM | 4077 | CD2 | TYR | C | 71 | 44.332 | 109.963 | −6.011 | 1.00 | 100.76 | 6 |
| ATOM | 4078 | CE2 | TYR | C | 71 | 43.061 | 110.437 | −5.672 | 1.00 | 101.32 | 6 |
| ATOM | 4079 | CZ | TYR | C | 71 | 42.160 | 109.592 | −5.045 | 1.00 | 101.36 | 6 |
| ATOM | 4080 | OH | TYR | C | 71 | 40.894 | 110.046 | −4.732 | 1.00 | 100.65 | 8 |
| ATOM | 4081 | C | TYR | C | 71 | 44.721 | 106.725 | −7.905 | 1.00 | 40.46 | 6 |
| ATOM | 4082 | O | TYR | C | 71 | 43.591 | 107.193 | −8.038 | 1.00 | 39.16 | 8 |
| ATOM | 4083 | N | ARG | C | 72 | 44.944 | 105.419 | −7.911 | 1.00 | 55.34 | 7 |
| ATOM | 4084 | CA | ARG | C | 72 | 43.812 | 104.538 | −8.107 | 1.00 | 55.85 | 6 |
| ATOM | 4085 | CB | ARG | C | 72 | 43.737 | 104.137 | −9.585 | 1.00 | 97.22 | 6 |
| ATOM | 4086 | CG | ARG | C | 72 | 42.499 | 103.325 | −9.949 | 1.00 | 99.67 | 6 |
| ATOM | 4087 | CD | ARG | C | 72 | 42.447 | 102.996 | −11.431 | 1.00 | 100.61 | 6 |
| ATOM | 4088 | NE | ARG | C | 72 | 41.858 | 101.682 | −11.663 | 1.00 | 102.71 | 7 |
| ATOM | 4089 | CZ | ARG | C | 72 | 41.921 | 101.022 | −12.815 | 1.00 | 104.14 | 6 |
| ATOM | 4090 | NH1 | ARG | C | 72 | 42.548 | 101.551 | −13.856 | 1.00 | 103.10 | 7 |
| ATOM | 4091 | NH2 | ARG | C | 72 | 41.366 | 99.820 | −12.921 | 1.00 | 105.69 | 7 |
| ATOM | 4092 | C | ARG | C | 72 | 43.736 | 103.295 | −7.230 | 1.00 | 55.38 | 6 |
| ATOM | 4093 | O | ARG | C | 72 | 44.406 | 102.293 | −7.487 | 1.00 | 55.48 | 8 |
| ATOM | 4094 | N | ILE | C | 73 | 42.924 | 103.368 | −6.184 | 1.00 | 45.89 | 7 |
| ATOM | 4095 | CA | ILE | C | 73 | 42.716 | 102.212 | −5.321 | 1.00 | 46.35 | 6 |
| ATOM | 4096 | CB | ILE | C | 73 | 42.063 | 102.615 | −3.953 | 1.00 | 79.44 | 6 |
| ATOM | 4097 | CG2 | ILE | C | 73 | 41.576 | 101.386 | −3.206 | 1.00 | 79.78 | 6 |
| ATOM | 4098 | CG1 | ILE | C | 73 | 43.061 | 103.371 | −3.082 | 1.00 | 79.96 | 6 |
| ATOM | 4099 | CD1 | ILE | C | 73 | 43.415 | 104.721 | −3.606 | 1.00 | 82.19 | 6 |
| ATOM | 4100 | C | ILE | C | 73 | 41.683 | 101.486 | −6.171 | 1.00 | 45.72 | 6 |
| ATOM | 4101 | O | ILE | C | 73 | 41.190 | 102.072 | −7.124 | 1.00 | 45.98 | 8 |
| ATOM | 4102 | N | GLY | C | 74 | 41.347 | 100.238 | −5.877 | 1.00 | 59.70 | 7 |
| ATOM | 4103 | CA | GLY | C | 74 | 40.325 | 99.618 | −6.699 | 1.00 | 59.89 | 6 |
| ATOM | 4104 | C | GLY | C | 74 | 40.210 | 98.115 | −6.685 | 1.00 | 59.31 | 6 |
| ATOM | 4105 | O | GLY | C | 74 | 40.582 | 97.464 | −5.715 | 1.00 | 59.22 | 8 |
| ATOM | 4106 | N | ASP | C | 75 | 39.676 | 97.580 | −7.780 | 1.00 | 75.15 | 7 |
| ATOM | 4107 | CA | ASP | C | 75 | 39.494 | 96.144 | −7.966 | 1.00 | 74.49 | 6 |
| ATOM | 4108 | CB | ASP | C | 75 | 39.890 | 95.732 | −9.426 | 1.00 | 60.41 | 6 |
| ATOM | 4109 | CG | ASP | C | 75 | 40.785 | 96.777 | −10.166 | 1.00 | 61.12 | 6 |
| ATOM | 4110 | OD1 | ASP | C | 75 | 40.351 | 97.924 | −10.406 | 1.00 | 61.97 | 8 |
| ATOM | 4111 | OD2 | ASP | C | 75 | 41.926 | 96.433 | −10.540 | 1.00 | 60.97 | 8 |
| ATOM | 4112 | C | ASP | C | 75 | 40.230 | 95.254 | −6.930 | 1.00 | 73.41 | 6 |
| ATOM | 4113 | O | ASP | C | 75 | 41.259 | 94.642 | −7.233 | 1.00 | 73.61 | 8 |
| ATOM | 4114 | N | PRO | C | 76 | 39.690 | 95.157 | −5.697 | 1.00 | 53.20 | 7 |
| ATOM | 4115 | CD | PRO | C | 76 | 38.342 | 95.596 | −5.308 | 1.00 | 76.69 | 6 |
| ATOM | 4116 | CA | PRO | C | 76 | 40.293 | 94.349 | −4.626 | 1.00 | 52.08 | 6 |
| ATOM | 4117 | CB | PRO | C | 76 | 39.281 | 94.456 | −3.495 | 1.00 | 75.29 | 6 |
| ATOM | 4118 | CG | PRO | C | 76 | 38.002 | 94.598 | −4.222 | 1.00 | 76.95 | 6 |
| ATOM | 4119 | C | PRO | C | 76 | 40.535 | 92.907 | −5.039 | 1.00 | 50.99 | 6 |
| ATOM | 4120 | O | PRO | C | 76 | 39.833 | 92.378 | −5.887 | 1.00 | 52.01 | 8 |
| ATOM | 4121 | N | PRO | C | 77 | 41.541 | 92.253 | −4.441 | 1.00 | 43.95 | 7 |
| ATOM | 4122 | CD | PRO | C | 77 | 42.534 | 92.871 | −3.546 | 1.00 | 100.31 | 6 |
| ATOM | 4123 | CA | PRO | C | 77 | 41.910 | 90.863 | −4.729 | 1.00 | 42.97 | 6 |
| ATOM | 4124 | CB | PRO | C | 77 | 43.053 | 90.605 | −3.752 | 1.00 | 99.15 | 6 |
| ATOM | 4125 | CG | PRO | C | 77 | 43.722 | 91.936 | −3.691 | 1.00 | 100.25 | 6 |
| ATOM | 4126 | C | PRO | C | 77 | 40.805 | 89.821 | −4.615 | 1.00 | 42.68 | 6 |
| ATOM | 4127 | O | PRO | C | 77 | 40.045 | 89.616 | −5.559 | 1.00 | 42.66 | 8 |
| ATOM | 4128 | N | PHE | C | 78 | 40.726 | 89.162 | −3.460 | 1.00 | 105.15 | 7 |
| ATOM | 4129 | CA | PHE | C | 78 | 39.736 | 88.111 | −3.235 | 1.00 | 104.28 | 6 |
| ATOM | 4130 | CB | PHE | C | 78 | 40.370 | 86.947 | −2.479 | 1.00 | 44.72 | 6 |
| ATOM | 4131 | CG | PHE | C | 78 | 41.528 | 86.341 | −3.185 | 1.00 | 41.61 | 6 |
| ATOM | 4132 | CD1 | PHE | C | 78 | 42.663 | 87.091 | −3.439 | 1.00 | 40.07 | 6 |
| ATOM | 4133 | CD2 | PHE | C | 78 | 41.479 | 85.023 | −3.611 | 1.00 | 40.88 | 6 |
| ATOM | 4134 | CE1 | PHE | C | 78 | 43.735 | 86.542 | −4.111 | 1.00 | 40.12 | 6 |
| ATOM | 4135 | CE2 | PHE | C | 78 | 42.544 | 84.456 | −4.284 | 1.00 | 41.19 | 6 |
| ATOM | 4136 | CZ | PHE | C | 78 | 43.680 | 85.216 | −4.537 | 1.00 | 40.97 | 6 |
| ATOM | 4137 | C | PHE | C | 78 | 38.513 | 88.571 | −2.478 | 1.00 | 104.94 | 6 |
| ATOM | 4138 | O | PHE | C | 78 | 37.586 | 89.119 | −3.068 | 1.00 | 106.80 | 8 |
| ATOM | 4139 | N | SER | C | 79 | 38.510 | 88.331 | −1.170 | 1.00 | 30.96 | 7 |
| ATOM | 4140 | CA | SER | C | 79 | 37.382 | 88.729 | −0.336 | 1.00 | 31.37 | 6 |
| ATOM | 4141 | CB | SER | C | 79 | 36.118 | 88.019 | −0.803 | 1.00 | 97.02 | 6 |
| ATOM | 4142 | OG | SER | C | 79 | 36.158 | 86.651 | −0.430 | 1.00 | 95.83 | 8 |
| ATOM | 4143 | C | SER | C | 79 | 37.556 | 88.437 | 1.145 | 1.00 | 32.21 | 6 |
| ATOM | 4144 | O | SER | C | 79 | 38.066 | 87.393 | 1.514 | 1.00 | 32.04 | 8 |
| ATOM | 4145 | N | GLN | C | 80 | 37.113 | 89.353 | 1.997 | 1.00 | 71.63 | 7 |
| ATOM | 4146 | CA | GLN | C | 80 | 37.202 | 89.121 | 3.429 | 1.00 | 73.84 | 6 |
| ATOM | 4147 | CB | GLN | C | 80 | 36.353 | 90.134 | 4.188 | 1.00 | 75.43 | 6 |
| ATOM | 4148 | CG | GLN | C | 80 | 36.884 | 91.544 | 4.141 | 1.00 | 75.54 | 6 |
| ATOM | 4149 | CD | GLN | C | 80 | 36.155 | 92.451 | 5.104 | 1.00 | 75.38 | 6 |
| ATOM | 4150 | OE1 | GLN | C | 80 | 35.547 | 91.989 | 6.067 | 1.00 | 74.86 | 8 |
| ATOM | 4151 | NE2 | GLN | C | 80 | 36.227 | 93.752 | 4.862 | 1.00 | 75.75 | 7 |
| ATOM | 4152 | C | GLN | C | 80 | 36.653 | 87.715 | 3.660 | 1.00 | 74.89 | 6 |
| ATOM | 4153 | O | GLN | C | 80 | 35.756 | 87.278 | 2.936 | 1.00 | 74.90 | 8 |
| ATOM | 4154 | N | ASP | C | 81 | 37.184 | 87.016 | 4.661 | 1.00 | 54.21 | 7 |
| ATOM | 4155 | CA | ASP | C | 81 | 36.764 | 85.645 | 4.961 | 1.00 | 55.86 | 6 |
| ATOM | 4156 | CB | ASP | C | 81 | 35.256 | 85.457 | 4.708 | 1.00 | 95.24 | 6 |
| ATOM | 4157 | CG | ASP | C | 81 | 34.424 | 85.514 | 5.985 | 1.00 | 96.12 | 6 |
| ATOM | 4158 | OD1 | ASP | C | 81 | 33.181 | 85.604 | 5.875 | 1.00 | 95.54 | 8 |
| ATOM | 4159 | OD2 | ASP | C | 81 | 35.001 | 85.453 | 7.095 | 1.00 | 96.33 | 8 |
| ATOM | 4160 | C | ASP | C | 81 | 37.560 | 84.738 | 4.031 | 1.00 | 56.84 | 6 |
| ATOM | 4161 | O | ASP | C | 81 | 38.147 | 83.743 | 4.462 | 1.00 | 57.38 | 8 |
| ATOM | 4162 | N | GLU | C | 82 | 37.568 | 85.097 | 2.749 | 1.00 | 54.96 | 7 |
| ATOM | 4163 | CA | GLU | C | 82 | 38.301 | 84.354 | 1.729 | 1.00 | 56.17 | 6 |
| ATOM | 4164 | CB | GLU | C | 82 | 37.992 | 84.933 | 0.347 | 1.00 | 93.25 | 6 |
| ATOM | 4165 | CG | GLU | C | 82 | 38.317 | 84.045 | −0.837 | 1.00 | 95.92 | 6 |
| ATOM | 4166 | CD | GLU | C | 82 | 38.033 | 84.733 | −2.169 | 1.00 | 97.60 | 6 |
| ATOM | 4167 | OE1 | GLU | C | 82 | 37.052 | 85.501 | −2.254 | 1.00 | 96.68 | 8 |
| ATOM | 4168 | OE2 | GLU | C | 82 | 38.783 | 84.497 | −3.142 | 1.00 | 99.04 | 8 |
| ATOM | 4169 | C | GLU | C | 82 | 39.773 | 84.567 | 2.062 | 1.00 | 56.43 | 6 |
| ATOM | 4170 | O | GLU | C | 82 | 40.643 | 83.786 | 1.670 | 1.00 | 56.69 | 8 |
| ATOM | 4171 | N | CYS | C | 83 | 40.035 | 85.643 | 2.800 | 1.00 | 46.10 | 7 |
| ATOM | 4172 | CA | CYS | C | 83 | 41.387 | 85.992 | 3.195 | 1.00 | 46.27 | 6 |
| ATOM | 4173 | CB | CYS | C | 83 | 41.488 | 87.491 | 3.473 | 1.00 | 42.68 | 6 |
| ATOM | 4174 | SG | CYS | C | 83 | 42.080 | 88.510 | 2.098 | 1.00 | 46.55 | 16 |
| ATOM | 4175 | C | CYS | C | 83 | 41.767 | 85.219 | 4.435 | 1.00 | 46.32 | 6 |
| ATOM | 4176 | O | CYS | C | 83 | 42.624 | 84.341 | 4.384 | 1.00 | 47.41 | 8 |
| ATOM | 4177 | N | ARG | C | 84 | 41.115 | 85.549 | 5.546 | 1.00 | 84.05 | 7 |
| ATOM | 4178 | CA | ARG | C | 84 | 41.360 | 84.909 | 6.836 | 1.00 | 84.14 | 6 |
| ATOM | 4179 | CB | ARG | C | 84 | 40.213 | 85.248 | 7.788 | 1.00 | 63.56 | 6 |
| ATOM | 4180 | CG | ARG | C | 84 | 39.841 | 86.730 | 7.773 | 1.00 | 65.71 | 6 |
| ATOM | 4181 | CD | ARG | C | 84 | 38.499 | 87.020 | 8.456 | 1.00 | 66.90 | 6 |
| ATOM | 4182 | NE | ARG | C | 84 | 38.042 | 88.390 | 8.205 | 1.00 | 67.15 | 7 |
| ATOM | 4183 | CZ | ARG | C | 84 | 36.914 | 88.897 | 8.684 | 1.00 | 67.18 | 6 |
| ATOM | 4184 | NH1 | ARG | C | 84 | 36.131 | 88.145 | 9.436 | 1.00 | 67.91 | 7 |
| ATOM | 4185 | NH2 | ARG | C | 84 | 36.581 | 90.153 | 8.425 | 1.00 | 66.18 | 7 |
| ATOM | 4186 | C | ARG | C | 84 | 41.471 | 83.395 | 6.668 | 1.00 | 83.47 | 6 |
| ATOM | 4187 | O | ARG | C | 84 | 42.113 | 82.704 | 7.458 | 1.00 | 83.60 | 8 |
| ATOM | 4188 | N | GLU | C | 85 | 40.839 | 82.892 | 5.616 | 1.00 | 51.89 | 7 |
| ATOM | 4189 | CA | GLU | C | 85 | 40.845 | 81.473 | 5.317 | 1.00 | 50.77 | 6 |
| ATOM | 4190 | CB | GLU | C | 85 | 39.711 | 81.162 | 4.337 | 1.00 | 110.83 | 6 |
| ATOM | 4191 | CG | GLU | C | 85 | 39.405 | 79.694 | 4.168 | 1.00 | 114.03 | 6 |
| ATOM | 4192 | CD | GLU | C | 85 | 37.971 | 79.454 | 3.741 | 1.00 | 116.33 | 6 |
| ATOM | 4193 | OE1 | GLU | C | 85 | 37.566 | 79.951 | 2.665 | 1.00 | 117.61 | 8 |
| ATOM | 4194 | OE2 | GLU | C | 85 | 37.243 | 78.768 | 4.491 | 1.00 | 118.24 | 8 |
| ATOM | 4195 | C | GLU | C | 85 | 42.190 | 81.090 | 4.724 | 1.00 | 48.87 | 6 |
| ATOM | 4196 | O | GLU | C | 85 | 42.891 | 80.238 | 5.268 | 1.00 | 48.42 | 8 |
| ATOM | 4197 | N | LYS | C | 86 | 42.542 | 81.755 | 3.623 | 1.00 | 42.41 | 7 |
| ATOM | 4198 | CA | LYS | C | 86 | 43.788 | 81.515 | 2.891 | 1.00 | 41.09 | 6 |
| ATOM | 4199 | CB | LYS | C | 86 | 43.529 | 81.694 | 1.397 | 1.00 | 51.50 | 6 |
| ATOM | 4200 | CG | LYS | C | 86 | 42.664 | 80.608 | 0.806 | 1.00 | 51.79 | 6 |
| ATOM | 4201 | CD | LYS | C | 86 | 42.216 | 80.915 | −0.609 | 1.00 | 52.63 | 6 |
| ATOM | 4202 | CE | LYS | C | 86 | 41.368 | 79.761 | −1.135 | 1.00 | 53.06 | 6 |
| ATOM | 4203 | NZ | LYS | C | 86 | 40.609 | 80.092 | −2.375 | 1.00 | 52.14 | 7 |
| ATOM | 4204 | C | LYS | C | 86 | 44.974 | 82.391 | 3.312 | 1.00 | 40.46 | 6 |
| ATOM | 4205 | O | LYS | C | 86 | 45.927 | 82.576 | 2.541 | 1.00 | 39.36 | 8 |
| ATOM | 4206 | N | ASP | C | 87 | 44.912 | 82.909 | 4.541 | 1.00 | 49.99 | 7 |
| ATOM | 4207 | CA | ASP | C | 87 | 45.959 | 83.770 | 5.094 | 1.00 | 48.35 | 6 |
| ATOM | 4208 | CB | ASP | C | 87 | 47.226 | 82.961 | 5.387 | 1.00 | 56.86 | 6 |
| ATOM | 4209 | CG | ASP | C | 87 | 47.079 | 82.066 | 6.599 | 1.00 | 56.33 | 6 |
| ATOM | 4210 | OD1 | ASP | C | 87 | 46.566 | 82.549 | 7.625 | 1.00 | 56.05 | 8 |
| ATOM | 4211 | OD2 | ASP | C | 87 | 47.490 | 80.887 | 6.533 | 1.00 | 57.01 | 8 |
| ATOM | 4212 | C | ASP | C | 87 | 46.309 | 84.936 | 4.177 | 1.00 | 47.56 | 6 |
| ATOM | 4213 | O | ASP | C | 87 | 47.477 | 85.287 | 4.018 | 1.00 | 47.08 | 8 |
| ATOM | 4214 | N | LEU | C | 88 | 45.293 | 85.541 | 3.578 | 1.00 | 38.58 | 7 |
| ATOM | 4215 | CA | LEU | C | 88 | 45.526 | 86.659 | 2.690 | 1.00 | 37.19 | 6 |
| ATOM | 4216 | CB | LEU | C | 88 | 44.652 | 86.529 | 1.451 | 1.00 | 93.94 | 6 |
| ATOM | 4217 | CG | LEU | C | 88 | 44.885 | 85.243 | 0.658 | 1.00 | 94.32 | 6 |
| ATOM | 4218 | CD1 | LEU | C | 88 | 44.093 | 85.334 | −0.633 | 1.00 | 95.01 | 6 |
| ATOM | 4219 | CD2 | LEU | C | 88 | 46.372 | 85.040 | 0.364 | 1.00 | 94.63 | 6 |
| ATOM | 4220 | C | LEU | C | 88 | 45.277 | 87.994 | 3.376 | 1.00 | 36.02 | 6 |
| ATOM | 4221 | O | LEU | C | 88 | 44.302 | 88.161 | 4.098 | 1.00 | 35.88 | 8 |
| ATOM | 4222 | N | THR | C | 89 | 46.184 | 88.935 | 3.146 | 1.00 | 29.88 | 7 |
| ATOM | 4223 | CA | THR | C | 89 | 46.094 | 90.259 | 3.731 | 1.00 | 29.65 | 6 |
| ATOM | 4224 | CB | THR | C | 89 | 47.479 | 90.955 | 3.737 | 1.00 | 118.66 | 6 |
| ATOM | 4225 | OG1 | THR | C | 89 | 48.448 | 90.092 | 4.345 | 1.00 | 120.67 | 8 |
| ATOM | 4226 | CG2 | THR | C | 89 | 47.422 | 92.265 | 4.515 | 1.00 | 119.01 | 6 |
| ATOM | 4227 | C | THR | C | 89 | 45.129 | 91.101 | 2.906 | 1.00 | 28.47 | 6 |
| ATOM | 4228 | O | THR | C | 89 | 45.539 | 91.743 | 1.946 | 1.00 | 28.45 | 8 |
| ATOM | 4229 | N | TYR | C | 90 | 43.852 | 91.089 | 3.281 | 1.00 | 32.04 | 7 |
| ATOM | 4230 | CA | TYR | C | 90 | 42.807 | 91.852 | 2.595 | 1.00 | 30.45 | 6 |
| ATOM | 4231 | CB | TYR | C | 90 | 41.619 | 92.044 | 3.536 | 1.00 | 59.69 | 6 |
| ATOM | 4232 | CG | TYR | C | 90 | 40.472 | 92.807 | 2.931 | 1.00 | 58.99 | 6 |
| ATOM | 4233 | CD1 | TYR | C | 90 | 39.680 | 92.242 | 1.938 | 1.00 | 58.51 | 6 |
| ATOM | 4234 | CE1 | TYR | C | 90 | 38.638 | 92.949 | 1.364 | 1.00 | 58.19 | 6 |
| ATOM | 4235 | CD2 | TYR | C | 90 | 40.189 | 94.101 | 3.336 | 1.00 | 58.79 | 6 |
| ATOM | 4236 | CE2 | TYR | C | 90 | 39.152 | 94.813 | 2.769 | 1.00 | 58.09 | 6 |
| ATOM | 4237 | CZ | TYR | C | 90 | 38.382 | 94.234 | 1.785 | 1.00 | 57.73 | 6 |
| ATOM | 4238 | OH | TYR | C | 90 | 37.358 | 94.956 | 1.225 | 1.00 | 58.50 | 8 |
| ATOM | 4239 | C | TYR | C | 90 | 43.308 | 93.218 | 2.149 | 1.00 | 30.34 | 6 |
| ATOM | 4240 | O | TYR | C | 90 | 43.250 | 94.183 | 2.917 | 1.00 | 29.90 | 8 |
| ATOM | 4241 | N | GLN | C | 91 | 43.789 | 93.298 | 0.909 | 1.00 | 80.36 | 7 |
| ATOM | 4242 | CA | GLN | C | 91 | 44.322 | 94.540 | 0.357 | 1.00 | 80.21 | 6 |
| ATOM | 4243 | CB | GLN | C | 91 | 45.760 | 94.330 | −0.108 | 1.00 | 69.15 | 6 |
| ATOM | 4244 | CG | GLN | C | 91 | 46.719 | 93.994 | 1.005 | 1.00 | 70.45 | 6 |
| ATOM | 4245 | CD | GLN | C | 91 | 48.044 | 93.506 | 0.481 | 1.00 | 71.04 | 6 |
| ATOM | 4246 | OE1 | GLN | C | 91 | 48.086 | 92.670 | −0.422 | 1.00 | 70.94 | 8 |
| ATOM | 4247 | NE2 | GLN | C | 91 | 49.140 | 94.014 | 1.049 | 1.00 | 71.35 | 7 |
| ATOM | 4248 | C | GLN | C | 91 | 43.506 | 95.051 | −0.812 | 1.00 | 79.27 | 6 |
| ATOM | 4249 | O | GLN | C | 91 | 42.302 | 94.845 | −0.875 | 1.00 | 79.86 | 8 |
| ATOM | 4250 | N | ALA | C | 92 | 44.180 | 95.728 | −1.734 | 1.00 | 69.38 | 7 |
| ATOM | 4251 | CA | ALA | C | 92 | 43.550 | 96.274 | −2.931 | 1.00 | 69.05 | 6 |
| ATOM | 4252 | CB | ALA | C | 92 | 42.455 | 97.270 | −2.552 | 1.00 | 13.87 | 6 |
| ATOM | 4253 | C | ALA | C | 92 | 44.610 | 96.946 | −3.805 | 1.00 | 68.88 | 6 |
| ATOM | 4254 | O | ALA | C | 92 | 45.516 | 97.616 | −3.305 | 1.00 | 68.52 | 8 |
| ATOM | 4255 | N | PRO | C | 93 | 44.507 | 96.771 | −5.128 | 1.00 | 48.77 | 7 |
| ATOM | 4256 | CD | PRO | C | 93 | 43.396 | 96.091 | −5.813 | 1.00 | 110.54 | 6 |
| ATOM | 4257 | CA | PRO | C | 93 | 45.441 | 97.342 | −6.100 | 1.00 | 48.32 | 6 |
| ATOM | 4258 | CB | PRO | C | 93 | 44.984 | 96.721 | −7.413 | 1.00 | 110.46 | 6 |
| ATOM | 4259 | CG | PRO | C | 93 | 43.506 | 96.638 | −7.219 | 1.00 | 111.39 | 6 |
| ATOM | 4260 | C | PRO | C | 93 | 45.443 | 98.869 | −6.162 | 1.00 | 47.83 | 6 |
| ATOM | 4261 | O | PRO | C | 93 | 44.432 | 99.506 | −6.501 | 1.00 | 48.40 | 8 |
| ATOM | 4262 | N | LEU | C | 94 | 46.589 | 99.453 | −5.839 | 1.00 | 48.05 | 7 |
| ATOM | 4263 | CA | LEU | C | 94 | 46.722 | 100.894 | −5.878 | 1.00 | 46.69 | 6 |
| ATOM | 4264 | CB | LEU | C | 94 | 47.059 | 101.446 | −4.485 | 1.00 | 79.69 | 6 |
| ATOM | 4265 | CG | LEU | C | 94 | 47.462 | 102.932 | −4.429 | 1.00 | 79.83 | 6 |
| ATOM | 4266 | CD1 | LEU | C | 94 | 46.313 | 103.800 | −4.898 | 1.00 | 79.99 | 6 |
| ATOM | 4267 | CD2 | LEU | C | 94 | 47.854 | 103.318 | −3.022 | 1.00 | 79.90 | 6 |
| ATOM | 4268 | C | LEU | C | 94 | 47.796 | 101.337 | −6.859 | 1.00 | 45.26 | 6 |
| ATOM | 4269 | O | LEU | C | 94 | 48.969 | 101.386 | −6.503 | 1.00 | 44.54 | 8 |
| ATOM | 4270 | N | TYR | C | 95 | 47.416 | 101.644 | −8.096 | 1.00 | 34.68 | 7 |
| ATOM | 4271 | CA | TYR | C | 95 | 48.411 | 102.135 | −9.042 | 1.00 | 34.95 | 6 |
| ATOM | 4272 | CB | TYR | C | 95 | 48.266 | 101.458 | −10.425 | 1.00 | 51.57 | 6 |
| ATOM | 4273 | CG | TYR | C | 95 | 47.026 | 100.607 | −10.637 | 1.00 | 52.57 | 6 |
| ATOM | 4274 | CD1 | TYR | C | 95 | 45.768 | 101.190 | −10.768 | 1.00 | 53.23 | 6 |
| ATOM | 4275 | CE1 | TYR | C | 95 | 44.624 | 100.414 | −10.987 | 1.00 | 52.69 | 6 |
| ATOM | 4276 | CD2 | TYR | C | 95 | 47.118 | 99.214 | −10.726 | 1.00 | 53.25 | 6 |
| ATOM | 4277 | CE2 | TYR | C | 95 | 45.975 | 98.418 | −10.939 | 1.00 | 53.54 | 6 |
| ATOM | 4278 | CZ | TYR | C | 95 | 44.725 | 99.030 | −11.069 | 1.00 | 52.95 | 6 |
| ATOM | 4279 | OH | TYR | C | 95 | 43.579 | 98.269 | −11.273 | 1.00 | 53.39 | 8 |
| ATOM | 4280 | C | TYR | C | 95 | 48.287 | 103.675 | −9.148 | 1.00 | 34.90 | 6 |
| ATOM | 4281 | O | TYR | C | 95 | 47.259 | 104.244 | −8.759 | 1.00 | 33.96 | 8 |
| ATOM | 4282 | N | ALA | C | 96 | 49.333 | 104.342 | −9.647 | 1.00 | 29.90 | 7 |
| ATOM | 4283 | CA | ALA | C | 96 | 49.324 | 105.802 | −9.780 | 1.00 | 31.71 | 6 |
| ATOM | 4284 | CB | ALA | C | 96 | 50.085 | 106.414 | −8.621 | 1.00 | 108.85 | 6 |
| ATOM | 4285 | C | ALA | C | 96 | 49.881 | 106.341 | −11.109 | 1.00 | 33.12 | 6 |
| ATOM | 4286 | O | ALA | C | 96 | 51.021 | 106.065 | −11.468 | 1.00 | 33.20 | 8 |
| ATOM | 4287 | N | ARG | C | 97 | 49.077 | 107.144 | −11.807 | 1.00 | 56.27 | 7 |
| ATOM | 4288 | CA | ARG | C | 97 | 49.432 | 107.744 | −13.108 | 1.00 | 57.36 | 6 |
| ATOM | 4289 | CB | ARG | C | 97 | 48.184 | 108.368 | −13.735 | 1.00 | 69.99 | 6 |
| ATOM | 4290 | CG | ARG | C | 97 | 46.984 | 107.450 | −13.842 | 1.00 | 71.50 | 6 |
| ATOM | 4291 | CD | ARG | C | 97 | 45.935 | 108.071 | −14.752 | 1.00 | 72.07 | 6 |
| ATOM | 4292 | NE | ARG | C | 97 | 46.567 | 108.672 | −15.922 | 1.00 | 73.73 | 7 |
| ATOM | 4293 | CZ | ARG | C | 97 | 45.930 | 108.991 | −17.041 | 1.00 | 74.74 | 6 |
| ATOM | 4294 | NH1 | ARG | C | 97 | 44.629 | 108.762 | −17.151 | 1.00 | 75.60 | 7 |
| ATOM | 4295 | NH2 | ARG | C | 97 | 46.596 | 109.540 | −18.049 | 1.00 | 74.96 | 7 |
| ATOM | 4296 | C | ARG | C | 97 | 50.528 | 108.823 | −13.116 | 1.00 | 57.68 | 6 |
| ATOM | 4297 | O | ARG | C | 97 | 50.278 | 109.958 | −12.710 | 1.00 | 56.82 | 8 |
| ATOM | 4298 | N | LEU | C | 98 | 51.716 | 108.495 | −13.618 | 1.00 | 102.03 | 7 |
| ATOM | 4299 | CA | LEU | C | 98 | 52.818 | 109.467 | −13.668 | 1.00 | 103.88 | 6 |
| ATOM | 4300 | CB | LEU | C | 98 | 54.054 | 108.888 | −12.963 | 1.00 | 55.34 | 6 |
| ATOM | 4301 | CG | LEU | C | 98 | 53.766 | 107.917 | −11.803 | 1.00 | 55.72 | 6 |
| ATOM | 4302 | CD1 | LEU | C | 98 | 55.067 | 107.471 | −11.165 | 1.00 | 56.45 | 6 |
| ATOM | 4303 | CD2 | LEU | C | 98 | 52.866 | 108.572 | −10.770 | 1.00 | 54.94 | 6 |
| ATOM | 4304 | C | LEU | C | 98 | 53.151 | 109.833 | −15.125 | 1.00 | 104.43 | 6 |
| ATOM | 4305 | O | LEU | C | 98 | 52.603 | 109.236 | −16.050 | 1.00 | 105.22 | 8 |
| ATOM | 4306 | N | GLN | C | 99 | 54.034 | 110.810 | −15.332 | 1.00 | 65.41 | 7 |
| ATOM | 4307 | CA | GLN | C | 99 | 54.407 | 111.243 | −16.688 | 1.00 | 65.55 | 6 |
| ATOM | 4308 | CB | GLN | C | 99 | 53.305 | 112.132 | −17.289 | 1.00 | 54.10 | 6 |
| ATOM | 4309 | CG | GLN | C | 99 | 52.742 | 113.191 | −16.329 | 1.00 | 54.45 | 6 |
| ATOM | 4310 | CD | GLN | C | 99 | 51.745 | 114.173 | −16.975 | 1.00 | 54.22 | 6 |
| ATOM | 4311 | OE1 | GLN | C | 99 | 50.946 | 113.799 | −17.836 | 1.00 | 53.31 | 8 |
| ATOM | 4312 | NE2 | GLN | C | 99 | 51.781 | 115.432 | −16.530 | 1.00 | 53.03 | 7 |
| ATOM | 4313 | C | GLN | C | 99 | 55.724 | 112.009 | −16.719 | 1.00 | 66.35 | 6 |
| ATOM | 4314 | O | GLN | C | 99 | 55.746 | 113.204 | −16.432 | 1.00 | 67.40 | 8 |
| ATOM | 4315 | N | LEU | C | 100 | 56.815 | 111.337 | −17.083 | 1.00 | 57.89 | 7 |
| ATOM | 4316 | CA | LEU | C | 100 | 58.134 | 111.988 | −17.121 | 1.00 | 58.25 | 6 |
| ATOM | 4317 | CB | LEU | C | 100 | 59.253 | 110.949 | −17.182 | 1.00 | 58.65 | 6 |
| ATOM | 4318 | CG | LEU | C | 100 | 58.824 | 109.541 | −17.579 | 1.00 | 59.06 | 6 |
| ATOM | 4319 | CD1 | LEU | C | 100 | 58.250 | 109.558 | −18.983 | 1.00 | 59.44 | 6 |
| ATOM | 4320 | CD2 | LEU | C | 100 | 60.013 | 108.607 | −17.468 | 1.00 | 58.33 | 6 |
| ATOM | 4321 | C | LEU | C | 100 | 58.312 | 112.984 | −18.247 | 1.00 | 59.20 | 6 |
| ATOM | 4322 | O | LEU | C | 100 | 57.903 | 112.741 | −19.382 | 1.00 | 59.43 | 8 |
| ATOM | 4323 | N | ILE | C | 101 | 58.931 | 114.113 | −17.920 | 1.00 | 72.44 | 7 |
| ATOM | 4324 | CA | ILE | C | 101 | 59.157 | 115.174 | −18.891 | 1.00 | 74.12 | 6 |
| ATOM | 4325 | CB | ILE | C | 101 | 58.997 | 116.566 | −18.227 | 1.00 | 59.88 | 6 |
| ATOM | 4326 | CG2 | ILE | C | 101 | 58.873 | 117.647 | −19.292 | 1.00 | 59.71 | 6 |
| ATOM | 4327 | CG1 | ILE | C | 101 | 57.758 | 116.584 | −17.326 | 1.00 | 60.33 | 6 |
| ATOM | 4328 | CD1 | ILE | C | 101 | 56.461 | 116.261 | −18.039 | 1.00 | 61.69 | 6 |
| ATOM | 4329 | C | ILE | C | 101 | 60.563 | 115.050 | −19.479 | 1.00 | 75.54 | 6 |
| ATOM | 4330 | O | ILE | C | 101 | 61.377 | 114.275 | −18.986 | 1.00 | 75.14 | 8 |
| ATOM | 4331 | N | HIS | C | 102 | 60.835 | 115.810 | −20.538 | 1.00 | 74.04 | 7 |
| ATOM | 4332 | CA | HIS | C | 102 | 62.136 | 115.798 | −21.202 | 1.00 | 75.31 | 6 |
| ATOM | 4333 | CB | HIS | C | 102 | 62.010 | 115.223 | −22.619 | 1.00 | 83.57 | 6 |
| ATOM | 4334 | CG | HIS | C | 102 | 61.650 | 113.771 | −22.670 | 1.00 | 84.63 | 6 |
| ATOM | 4335 | CD2 | HIS | C | 102 | 60.613 | 113.131 | −23.260 | 1.00 | 84.82 | 6 |
| ATOM | 4336 | ND1 | HIS | C | 102 | 62.438 | 112.788 | −22.112 | 1.00 | 85.38 | 7 |
| ATOM | 4337 | CE1 | HIS | C | 102 | 61.903 | 111.604 | −22.358 | 1.00 | 85.82 | 6 |
| ATOM | 4338 | NE2 | HIS | C | 102 | 60.795 | 111.784 | −23.054 | 1.00 | 85.53 | 7 |
| ATOM | 4339 | C | HIS | C | 102 | 62.722 | 117.210 | −21.321 | 1.00 | 75.87 | 6 |
| ATOM | 4340 | O | HIS | C | 102 | 62.562 | 118.066 | −20.441 | 1.00 | 75.97 | 8 |
| ATOM | 4341 | N | LYS | C | 103 | 63.403 | 117.417 | −22.443 | 1.00 | 107.67 | 7 |
| ATOM | 4342 | CA | LYS | C | 103 | 64.040 | 118.676 | −22.813 | 1.00 | 108.49 | 6 |
| ATOM | 4343 | CB | LYS | C | 103 | 65.306 | 118.919 | −21.983 | 1.00 | 112.21 | 6 |
| ATOM | 4344 | CG | LYS | C | 103 | 65.185 | 120.085 | −21.008 | 1.00 | 113.26 | 6 |
| ATOM | 4345 | CD | LYS | C | 103 | 64.739 | 121.354 | −21.723 | 1.00 | 113.90 | 6 |
| ATOM | 4346 | CE | LYS | C | 103 | 64.448 | 122.480 | −20.744 | 1.00 | 114.86 | 6 |
| ATOM | 4347 | NZ | LYS | C | 103 | 63.685 | 123.587 | −21.394 | 1.00 | 115.30 | 7 |
| ATOM | 4348 | C | LYS | C | 103 | 64.404 | 118.508 | −24.286 | 1.00 | 108.56 | 6 |
| ATOM | 4349 | O | LYS | C | 103 | 65.258 | 119.216 | −24.830 | 1.00 | 108.10 | 8 |
| ATOM | 4350 | N | ASP | C | 104 | 63.727 | 117.548 | −24.911 | 1.00 | 51.37 | 7 |
| ATOM | 4351 | CA | ASP | C | 104 | 63.928 | 117.196 | −26.307 | 1.00 | 51.13 | 6 |
| ATOM | 4352 | CB | ASP | C | 104 | 65.022 | 116.117 | −26.414 | 1.00 | 80.96 | 6 |
| ATOM | 4353 | CG | ASP | C | 104 | 64.948 | 115.070 | −25.287 | 1.00 | 81.71 | 6 |
| ATOM | 4354 | OD1 | ASP | C | 104 | 64.879 | 115.445 | −24.095 | 1.00 | 81.16 | 8 |
| ATOM | 4355 | OD2 | ASP | C | 104 | 64.979 | 113.858 | −25.586 | 1.00 | 83.61 | 8 |
| ATOM | 4356 | C | ASP | C | 104 | 62.600 | 116.702 | −26.880 | 1.00 | 51.12 | 6 |
| ATOM | 4357 | O | ASP | C | 104 | 62.547 | 115.702 | −27.605 | 1.00 | 50.09 | 8 |
| ATOM | 4358 | N | THR | C | 105 | 61.532 | 117.422 | −26.534 | 1.00 | 153.67 | 7 |
| ATOM | 4359 | CA | THR | C | 105 | 60.174 | 117.115 | −26.984 | 1.00 | 156.13 | 6 |
| ATOM | 4360 | CB | THR | C | 105 | 60.056 | 117.234 | −28.533 | 1.00 | 130.99 | 6 |
| ATOM | 4361 | OG1 | THR | C | 105 | 60.166 | 118.612 | −28.915 | 1.00 | 131.67 | 8 |
| ATOM | 4362 | CG2 | THR | C | 105 | 58.714 | 116.681 | −29.027 | 1.00 | 130.54 | 6 |
| ATOM | 4363 | C | THR | C | 105 | 59.687 | 115.734 | −26.547 | 1.00 | 157.07 | 6 |
| ATOM | 4364 | O | THR | C | 105 | 60.143 | 114.707 | −27.063 | 1.00 | 157.74 | 8 |
| ATOM | 4365 | N | GLY | C | 106 | 58.753 | 115.718 | −25.595 | 1.00 | 118.28 | 7 |
| ATOM | 4366 | CA | GLY | C | 106 | 58.215 | 114.459 | −25.111 | 1.00 | 117.30 | 6 |
| ATOM | 4367 | C | GLY | C | 106 | 57.591 | 114.516 | −23.730 | 1.00 | 116.71 | 6 |
| ATOM | 4368 | O | GLY | C | 106 | 58.187 | 115.024 | −22.778 | 1.00 | 116.63 | 8 |
| ATOM | 4369 | N | LEU | C | 107 | 56.381 | 113.978 | −23.628 | 1.00 | 62.99 | 7 |
| ATOM | 4370 | CA | LEU | C | 107 | 55.641 | 113.940 | −22.373 | 1.00 | 62.42 | 6 |
| ATOM | 4371 | CB | LEU | C | 107 | 54.618 | 115.078 | −22.345 | 1.00 | 116.78 | 6 |
| ATOM | 4372 | CG | LEU | C | 107 | 55.122 | 116.457 | −22.787 | 1.00 | 116.42 | 6 |
| ATOM | 4373 | CD1 | LEU | C | 107 | 53.989 | 117.459 | −22.664 | 1.00 | 116.42 | 6 |
| ATOM | 4374 | CD2 | LEU | C | 107 | 56.303 | 116.890 | −21.940 | 1.00 | 116.89 | 6 |
| ATOM | 4375 | C | LEU | C | 107 | 54.931 | 112.583 | −22.236 | 1.00 | 62.45 | 6 |
| ATOM | 4376 | O | LEU | C | 107 | 53.711 | 112.475 | −22.414 | 1.00 | 61.96 | 8 |
| ATOM | 4377 | N | ILE | C | 108 | 55.717 | 111.557 | −21.919 | 1.00 | 85.98 | 7 |
| ATOM | 4378 | CA | ILE | C | 108 | 55.233 | 110.187 | −21.759 | 1.00 | 86.51 | 6 |
| ATOM | 4379 | CB | ILE | C | 108 | 56.420 | 109.207 | −21.687 | 1.00 | 53.58 | 6 |
| ATOM | 4380 | CG2 | ILE | C | 108 | 55.898 | 107.772 | −21.651 | 1.00 | 53.89 | 6 |
| ATOM | 4381 | CG1 | ILE | C | 108 | 57.363 | 109.437 | −22.882 | 1.00 | 53.03 | 6 |
| ATOM | 4382 | CD1 | ILE | C | 108 | 58.780 | 108.849 | −22.723 | 1.00 | 51.80 | 6 |
| ATOM | 4383 | C | ILE | C | 108 | 54.393 | 110.024 | −20.493 | 1.00 | 87.01 | 6 |
| ATOM | 4384 | O | ILE | C | 108 | 54.701 | 110.625 | −19.460 | 1.00 | 86.91 | 8 |
| ATOM | 4385 | N | LYS | C | 109 | 53.347 | 109.200 | −20.576 | 1.00 | 85.47 | 7 |
| ATOM | 4386 | CA | LYS | C | 109 | 52.447 | 108.964 | −19.443 | 1.00 | 86.15 | 6 |
| ATOM | 4387 | CB | LYS | C | 109 | 51.192 | 109.833 | −19.558 | 1.00 | 111.00 | 6 |
| ATOM | 4388 | CG | LYS | C | 109 | 51.422 | 111.320 | −19.741 | 1.00 | 112.77 | 6 |
| ATOM | 4389 | CD | LYS | C | 109 | 50.091 | 112.067 | −19.869 | 1.00 | 112.61 | 6 |
| ATOM | 4390 | CE | LYS | C | 109 | 49.182 | 111.816 | −18.664 | 1.00 | 113.05 | 6 |
| ATOM | 4391 | NZ | LYS | C | 109 | 47.901 | 112.576 | −18.738 | 1.00 | 112.68 | 7 |
| ATOM | 4392 | C | LYS | C | 109 | 51.967 | 107.523 | −19.336 | 1.00 | 86.43 | 6 |
| ATOM | 4393 | O | LYS | C | 109 | 50.963 | 107.180 | −19.959 | 1.00 | 86.97 | 8 |
| ATOM | 4394 | N | GLU | C | 110 | 52.649 | 106.684 | −18.555 | 1.00 | 106.12 | 7 |
| ATOM | 4395 | CA | GLU | C | 110 | 52.199 | 105.300 | −18.396 | 1.00 | 106.53 | 6 |
| ATOM | 4396 | CB | GLU | C | 110 | 53.252 | 104.422 | −17.717 | 1.00 | 127.12 | 6 |
| ATOM | 4397 | CG | GLU | C | 110 | 54.279 | 103.813 | −18.656 | 1.00 | 129.05 | 6 |
| ATOM | 4398 | CD | GLU | C | 110 | 53.646 | 103.074 | −19.818 | 1.00 | 130.82 | 6 |
| ATOM | 4399 | OE1 | GLU | C | 110 | 52.645 | 102.355 | −19.594 | 1.00 | 131.80 | 8 |
| ATOM | 4400 | OE2 | GLU | C | 110 | 54.156 | 103.203 | −20.955 | 1.00 | 131.20 | 8 |
| ATOM | 4401 | C | GLU | C | 110 | 50.941 | 105.288 | −17.545 | 1.00 | 106.57 | 6 |
| ATOM | 4402 | O | GLU | C | 110 | 50.976 | 105.636 | −16.363 | 1.00 | 106.27 | 8 |
| ATOM | 4403 | N | ASP | C | 111 | 49.829 | 104.889 | −18.153 | 1.00 | 130.67 | 7 |
| ATOM | 4404 | CA | ASP | C | 111 | 48.562 | 104.837 | −17.441 | 1.00 | 130.36 | 6 |
| ATOM | 4405 | CB | ASP | C | 111 | 47.443 | 104.314 | −18.349 | 1.00 | 111.53 | 6 |
| ATOM | 4406 | CG | ASP | C | 111 | 47.143 | 105.246 | −19.500 | 1.00 | 112.76 | 6 |
| ATOM | 4407 | OD1 | ASP | C | 111 | 46.999 | 106.462 | −19.257 | 1.00 | 112.10 | 8 |
| ATOM | 4408 | OD2 | ASP | C | 111 | 47.042 | 104.763 | −20.647 | 1.00 | 114.07 | 8 |
| ATOM | 4409 | C | ASP | C | 111 | 48.671 | 103.945 | −16.218 | 1.00 | 129.01 | 6 |
| ATOM | 4410 | O | ASP | C | 111 | 49.320 | 102.900 | −16.251 | 1.00 | 128.94 | 8 |
| ATOM | 4411 | N | GLU | C | 112 | 48.038 | 104.380 | −15.137 | 1.00 | 65.59 | 7 |
| ATOM | 4412 | CA | GLU | C | 112 | 48.023 | 103.633 | −13.896 | 1.00 | 63.56 | 6 |
| ATOM | 4413 | CB | GLU | C | 112 | 47.309 | 102.295 | −14.101 | 1.00 | 52.11 | 6 |
| ATOM | 4414 | CG | GLU | C | 112 | 45.940 | 102.411 | −14.737 | 1.00 | 51.24 | 6 |
| ATOM | 4415 | CD | GLU | C | 112 | 44.939 | 103.138 | −13.859 | 1.00 | 49.80 | 6 |
| ATOM | 4416 | OE1 | GLU | C | 112 | 44.595 | 102.600 | −12.786 | 1.00 | 48.02 | 8 |
| ATOM | 4417 | OE2 | GLU | C | 112 | 44.494 | 104.242 | −14.247 | 1.00 | 48.94 | 8 |
| ATOM | 4418 | C | GLU | C | 112 | 49.404 | 103.371 | −13.320 | 1.00 | 62.52 | 6 |
| ATOM | 4419 | O | GLU | C | 112 | 49.667 | 103.729 | −12.174 | 1.00 | 63.01 | 8 |
| ATOM | 4420 | N | VAL | C | 113 | 50.288 | 102.757 | −14.106 | 1.00 | 27.09 | 7 |
| ATOM | 4421 | CA | VAL | C | 113 | 51.614 | 102.422 | −13.601 | 1.00 | 26.32 | 6 |
| ATOM | 4422 | CB | VAL | C | 113 | 52.356 | 103.659 | −13.080 | 1.00 | 46.96 | 6 |
| ATOM | 4423 | CG1 | VAL | C | 113 | 53.837 | 103.351 | −12.890 | 1.00 | 47.29 | 6 |
| ATOM | 4424 | CG2 | VAL | C | 113 | 52.129 | 104.821 | −14.026 | 1.00 | 47.74 | 6 |
| ATOM | 4425 | C | VAL | C | 113 | 51.334 | 101.515 | −12.408 | 1.00 | 26.34 | 6 |
| ATOM | 4426 | O | VAL | C | 113 | 51.326 | 101.976 | −11.264 | 1.00 | 26.39 | 8 |
| ATOM | 4427 | N | PHE | C | 114 | 51.078 | 100.236 | −12.682 | 1.00 | 66.07 | 7 |
| ATOM | 4428 | CA | PHE | C | 114 | 50.770 | 99.254 | −11.642 | 1.00 | 67.05 | 6 |
| ATOM | 4429 | CB | PHE | C | 114 | 50.988 | 97.828 | −12.166 | 1.00 | 208.87 | 6 |
| ATOM | 4430 | CG | PHE | C | 114 | 50.006 | 97.420 | −13.235 | 1.00 | 208.87 | 6 |
| ATOM | 4431 | CD1 | PHE | C | 114 | 50.049 | 98.002 | −14.500 | 1.00 | 208.87 | 6 |
| ATOM | 4432 | CD2 | PHE | C | 114 | 49.014 | 96.480 | −12.966 | 1.00 | 208.87 | 6 |
| ATOM | 4433 | CE1 | PHE | C | 114 | 49.119 | 97.658 | −15.478 | 1.00 | 208.87 | 6 |
| ATOM | 4434 | CE2 | PHE | C | 114 | 48.078 | 96.129 | −13.940 | 1.00 | 208.87 | 6 |
| ATOM | 4435 | CZ | PHE | C | 114 | 48.131 | 96.720 | −15.198 | 1.00 | 208.87 | 6 |
| ATOM | 4436 | C | PHE | C | 114 | 51.626 | 99.516 | −10.423 | 1.00 | 66.08 | 6 |
| ATOM | 4437 | O | PHE | C | 114 | 52.782 | 99.913 | −10.552 | 1.00 | 65.65 | 8 |
| ATOM | 4438 | N | LEU | C | 115 | 51.075 | 99.284 | −9.238 | 1.00 | 110.47 | 7 |
| ATOM | 4439 | CA | LEU | C | 115 | 51.837 | 99.586 | −8.042 | 1.00 | 109.25 | 6 |
| ATOM | 4440 | CB | LEU | C | 115 | 51.597 | 101.061 | −7.725 | 1.00 | 37.12 | 6 |
| ATOM | 4441 | CG | LEU | C | 115 | 52.362 | 101.840 | −6.671 | 1.00 | 37.25 | 6 |
| ATOM | 4442 | CD1 | LEU | C | 115 | 53.678 | 101.201 | −6.264 | 1.00 | 37.12 | 6 |
| ATOM | 4443 | CD2 | LEU | C | 115 | 52.597 | 103.196 | −7.286 | 1.00 | 38.33 | 6 |
| ATOM | 4444 | C | LEU | C | 115 | 51.578 | 98.695 | −6.820 | 1.00 | 108.54 | 6 |
| ATOM | 4445 | O | LEU | C | 115 | 51.036 | 97.589 | −6.948 | 1.00 | 108.51 | 8 |
| ATOM | 4446 | N | GLY | C | 116 | 51.991 | 99.185 | −5.647 | 1.00 | 46.71 | 7 |
| ATOM | 4447 | CA | GLY | C | 116 | 51.831 | 98.451 | −4.404 | 1.00 | 44.97 | 6 |
| ATOM | 4448 | C | GLY | C | 116 | 50.410 | 98.185 | −3.926 | 1.00 | 43.86 | 6 |
| ATOM | 4449 | O | GLY | C | 116 | 49.435 | 98.802 | −4.368 | 1.00 | 43.96 | 8 |
| ATOM | 4450 | N | HIS | C | 117 | 50.301 | 97.245 | −2.997 | 1.00 | 38.74 | 7 |
| ATOM | 4451 | CA | HIS | C | 117 | 49.020 | 96.864 | −2.432 | 1.00 | 37.35 | 6 |
| ATOM | 4452 | CB | HIS | C | 117 | 49.010 | 95.360 | −2.145 | 1.00 | 67.60 | 6 |
| ATOM | 4453 | CG | HIS | C | 117 | 48.884 | 94.510 | −3.372 | 1.00 | 68.06 | 6 |
| ATOM | 4454 | CD2 | HIS | C | 117 | 49.711 | 93.579 | −3.899 | 1.00 | 67.77 | 6 |
| ATOM | 4455 | ND1 | HIS | C | 117 | 47.778 | 94.549 | −4.195 | 1.00 | 68.70 | 7 |
| ATOM | 4456 | CE1 | HIS | C | 117 | 47.926 | 93.675 | −5.173 | 1.00 | 67.88 | 6 |
| ATOM | 4457 | NE2 | HIS | C | 117 | 49.091 | 93.074 | −5.017 | 1.00 | 68.90 | 7 |
| ATOM | 4458 | C | HIS | C | 117 | 48.730 | 97.641 | −1.160 | 1.00 | 35.72 | 6 |
| ATOM | 4459 | O | HIS | C | 117 | 49.639 | 97.971 | −0.400 | 1.00 | 34.73 | 8 |
| ATOM | 4460 | N | LEU | C | 118 | 47.456 | 97.928 | −0.931 | 1.00 | 74.84 | 7 |
| ATOM | 4461 | CA | LEU | C | 118 | 47.047 | 98.673 | 0.249 | 1.00 | 74.20 | 6 |
| ATOM | 4462 | CB | LEU | C | 118 | 46.449 | 100.027 | −0.161 | 1.00 | 36.96 | 6 |
| ATOM | 4463 | CG | LEU | C | 118 | 45.850 | 101.004 | 0.861 | 1.00 | 36.31 | 6 |
| ATOM | 4464 | CD1 | LEU | C | 118 | 45.481 | 102.272 | 0.131 | 1.00 | 36.65 | 6 |
| ATOM | 4465 | CD2 | LEU | C | 118 | 44.619 | 100.426 | 1.531 | 1.00 | 35.26 | 6 |
| ATOM | 4466 | C | LEU | C | 118 | 46.017 | 97.889 | 1.037 | 1.00 | 73.30 | 6 |
| ATOM | 4467 | O | LEU | C | 118 | 44.989 | 97.482 | 0.492 | 1.00 | 73.60 | 8 |
| ATOM | 4468 | N | PRO | C | 119 | 46.292 | 97.637 | 2.326 | 1.00 | 28.71 | 7 |
| ATOM | 4469 | CD | PRO | C | 119 | 47.628 | 97.637 | 2.960 | 1.00 | 31.16 | 6 |
| ATOM | 4470 | CA | PRO | C | 119 | 45.306 | 96.892 | 3.119 | 1.00 | 27.70 | 6 |
| ATOM | 4471 | CB | PRO | C | 119 | 46.105 | 96.455 | 4.359 | 1.00 | 31.28 | 6 |
| ATOM | 4472 | CG | PRO | C | 119 | 47.319 | 97.367 | 4.403 | 1.00 | 29.88 | 6 |
| ATOM | 4473 | C | PRO | C | 119 | 44.068 | 97.752 | 3.434 | 1.00 | 27.37 | 6 |
| ATOM | 4474 | O | PRO | C | 119 | 44.170 | 98.906 | 3.855 | 1.00 | 26.16 | 8 |
| ATOM | 4475 | N | LEU | C | 120 | 42.894 | 97.197 | 3.194 | 1.00 | 46.27 | 7 |
| ATOM | 4476 | CA | LEU | C | 120 | 41.687 | 97.952 | 3.437 | 1.00 | 47.57 | 6 |
| ATOM | 4477 | CB | LEU | C | 120 | 40.616 | 97.551 | 2.443 | 1.00 | 61.98 | 6 |
| ATOM | 4478 | CG | LEU | C | 120 | 40.905 | 98.073 | 1.042 | 1.00 | 62.57 | 6 |
| ATOM | 4479 | CD1 | LEU | C | 120 | 40.198 | 97.222 | 0.015 | 1.00 | 64.23 | 6 |
| ATOM | 4480 | CD2 | LEU | C | 120 | 40.470 | 99.524 | 0.947 | 1.00 | 61.87 | 6 |
| ATOM | 4481 | C | LEU | C | 120 | 41.197 | 97.747 | 4.845 | 1.00 | 48.67 | 6 |
| ATOM | 4482 | O | LEU | C | 120 | 41.637 | 96.827 | 5.534 | 1.00 | 48.90 | 8 |
| ATOM | 4483 | N | MET | C | 121 | 40.292 | 98.624 | 5.271 | 1.00 | 42.57 | 7 |
| ATOM | 4484 | CA | MET | C | 121 | 39.718 | 98.581 | 6.614 | 1.00 | 43.86 | 6 |
| ATOM | 4485 | CB | MET | C | 121 | 39.714 | 100.003 | 7.225 | 1.00 | 46.78 | 6 |
| ATOM | 4486 | CG | MET | C | 121 | 39.464 | 100.086 | 8.744 | 1.00 | 49.57 | 6 |
| ATOM | 4487 | SD | MET | C | 121 | 38.990 | 101.748 | 9.383 | 1.00 | 52.53 | 16 |
| ATOM | 4488 | CE | MET | C | 121 | 40.558 | 102.447 | 9.830 | 1.00 | 54.56 | 6 |
| ATOM | 4489 | C | MET | C | 121 | 38.292 | 98.046 | 6.483 | 1.00 | 43.88 | 6 |
| ATOM | 4490 | O | MET | C | 121 | 37.494 | 98.558 | 5.705 | 1.00 | 44.11 | 8 |
| ATOM | 4491 | N | THR | C | 122 | 37.973 | 97.001 | 7.229 | 1.00 | 29.41 | 7 |
| ATOM | 4492 | CA | THR | C | 122 | 36.638 | 96.441 | 7.171 | 1.00 | 30.53 | 6 |
| ATOM | 4493 | CB | THR | C | 122 | 36.553 | 95.186 | 7.993 | 1.00 | 59.28 | 6 |
| ATOM | 4494 | OG1 | THR | C | 122 | 37.695 | 94.376 | 7.717 | 1.00 | 59.40 | 8 |
| ATOM | 4495 | CG2 | THR | C | 122 | 35.290 | 94.423 | 7.643 | 1.00 | 61.33 | 6 |
| ATOM | 4496 | C | THR | C | 122 | 35.622 | 97.438 | 7.723 | 1.00 | 31.12 | 6 |
| ATOM | 4497 | O | THR | C | 122 | 35.999 | 98.406 | 8.386 | 1.00 | 31.55 | 8 |
| ATOM | 4498 | N | GLU | C | 123 | 34.338 | 97.198 | 7.456 | 1.00 | 49.45 | 7 |
| ATOM | 4499 | CA | GLU | C | 123 | 33.262 | 98.074 | 7.934 | 1.00 | 49.84 | 6 |
| ATOM | 4500 | CB | GLU | C | 123 | 31.908 | 97.434 | 7.631 | 1.00 | 90.22 | 6 |
| ATOM | 4501 | CG | GLU | C | 123 | 30.716 | 98.334 | 7.902 | 1.00 | 92.04 | 6 |
| ATOM | 4502 | CD | GLU | C | 123 | 29.398 | 97.686 | 7.518 | 1.00 | 92.73 | 6 |
| ATOM | 4503 | OE1 | GLU | C | 123 | 28.619 | 97.333 | 8.433 | 1.00 | 93.55 | 8 |
| ATOM | 4504 | OE2 | GLU | C | 123 | 29.150 | 97.523 | 6.301 | 1.00 | 93.00 | 8 |
| ATOM | 4505 | C | GLU | C | 123 | 33.417 | 98.265 | 9.439 | 1.00 | 49.89 | 6 |
| ATOM | 4506 | O | GLU | C | 123 | 33.117 | 99.326 | 9.995 | 1.00 | 49.12 | 8 |
| ATOM | 4507 | N | ASP | C | 124 | 33.898 | 97.193 | 10.063 | 1.00 | 52.75 | 7 |
| ATOM | 4508 | CA | ASP | C | 124 | 34.159 | 97.086 | 11.496 | 1.00 | 52.67 | 6 |
| ATOM | 4509 | CB | ASP | C | 124 | 34.775 | 95.708 | 11.784 | 1.00 | 61.98 | 6 |
| ATOM | 4510 | CG | ASP | C | 124 | 34.826 | 95.382 | 13.260 | 1.00 | 64.87 | 6 |
| ATOM | 4511 | OD1 | ASP | C | 124 | 34.951 | 96.323 | 14.071 | 1.00 | 67.98 | 8 |
| ATOM | 4512 | OD2 | ASP | C | 124 | 34.752 | 94.182 | 13.612 | 1.00 | 66.35 | 8 |
| ATOM | 4513 | C | ASP | C | 124 | 35.116 | 98.183 | 11.968 | 1.00 | 50.94 | 6 |
| ATOM | 4514 | O | ASP | C | 124 | 35.043 | 98.633 | 13.118 | 1.00 | 50.05 | 8 |
| ATOM | 4515 | N | GLY | C | 125 | 36.025 | 98.590 | 11.083 | 1.00 | 62.59 | 7 |
| ATOM | 4516 | CA | GLY | C | 125 | 36.986 | 99.628 | 11.420 | 1.00 | 61.39 | 6 |
| ATOM | 4517 | C | GLY | C | 125 | 38.375 | 99.110 | 11.745 | 1.00 | 59.43 | 6 |
| ATOM | 4518 | O | GLY | C | 125 | 39.121 | 99.708 | 12.520 | 1.00 | 59.88 | 8 |
| ATOM | 4519 | N | SER | C | 126 | 38.733 | 97.995 | 11.133 | 1.00 | 77.36 | 7 |
| ATOM | 4520 | CA | SER | C | 126 | 40.030 | 97.394 | 11.372 | 1.00 | 73.95 | 6 |
| ATOM | 4521 | CB | SER | C | 126 | 39.883 | 96.359 | 12.492 | 1.00 | 13.87 | 6 |
| ATOM | 4522 | OG | SER | C | 126 | 38.551 | 95.856 | 12.538 | 1.00 | 13.87 | 8 |
| ATOM | 4523 | C | SER | C | 126 | 40.512 | 96.749 | 10.072 | 1.00 | 73.71 | 6 |
| ATOM | 4524 | O | SER | C | 126 | 39.938 | 96.992 | 9.008 | 1.00 | 75.22 | 8 |
| ATOM | 4525 | N | PHE | C | 127 | 41.570 | 95.948 | 10.138 | 1.00 | 28.88 | 7 |
| ATOM | 4526 | CA | PHE | C | 127 | 42.044 | 95.290 | 8.937 | 1.00 | 27.11 | 6 |
| ATOM | 4527 | CB | PHE | C | 127 | 43.339 | 95.927 | 8.455 | 1.00 | 51.61 | 6 |
| ATOM | 4528 | CG | PHE | C | 127 | 43.347 | 97.420 | 8.516 | 1.00 | 53.45 | 6 |
| ATOM | 4529 | CD1 | PHE | C | 127 | 43.426 | 98.080 | 9.733 | 1.00 | 55.26 | 6 |
| ATOM | 4530 | CD2 | PHE | C | 127 | 43.328 | 98.174 | 7.355 | 1.00 | 54.19 | 6 |
| ATOM | 4531 | CE1 | PHE | C | 127 | 43.498 | 99.471 | 9.792 | 1.00 | 55.29 | 6 |
| ATOM | 4532 | CE2 | PHE | C | 127 | 43.398 | 99.566 | 7.404 | 1.00 | 55.13 | 6 |
| ATOM | 4533 | CZ | PHE | C | 127 | 43.483 | 100.213 | 8.624 | 1.00 | 55.33 | 6 |
| ATOM | 4534 | C | PHE | C | 127 | 42.291 | 93.806 | 9.193 | 1.00 | 26.15 | 6 |
| ATOM | 4535 | O | PHE | C | 127 | 42.443 | 93.387 | 10.348 | 1.00 | 26.14 | 8 |
| ATOM | 4536 | N | ILE | C | 128 | 42.298 | 93.020 | 8.113 | 1.00 | 29.00 | 7 |
| ATOM | 4537 | CA | ILE | C | 128 | 42.588 | 91.583 | 8.164 | 1.00 | 27.66 | 6 |
| ATOM | 4538 | CB | ILE | C | 128 | 41.754 | 90.738 | 7.214 | 1.00 | 15.94 | 6 |
| ATOM | 4539 | CG2 | ILE | C | 128 | 42.249 | 89.318 | 7.278 | 1.00 | 14.69 | 6 |
| ATOM | 4540 | CG1 | ILE | C | 128 | 40.274 | 90.828 | 7.556 | 1.00 | 17.08 | 6 |
| ATOM | 4541 | CD1 | ILE | C | 128 | 39.693 | 92.171 | 7.310 | 1.00 | 20.48 | 6 |
| ATOM | 4542 | C | ILE | C | 128 | 43.968 | 91.516 | 7.566 | 1.00 | 28.11 | 6 |
| ATOM | 4543 | O | ILE | C | 128 | 44.150 | 91.905 | 6.414 | 1.00 | 29.53 | 8 |
| ATOM | 4544 | N | ILE | C | 129 | 44.940 | 91.023 | 8.317 | 1.00 | 53.67 | 7 |
| ATOM | 4545 | CA | ILE | C | 129 | 46.284 | 90.964 | 7.780 | 1.00 | 52.88 | 6 |
| ATOM | 4546 | CB | ILE | C | 129 | 47.187 | 91.997 | 8.468 | 1.00 | 15.23 | 6 |
| ATOM | 4547 | CG2 | ILE | C | 129 | 48.547 | 91.971 | 7.843 | 1.00 | 16.20 | 6 |
| ATOM | 4548 | CG1 | ILE | C | 129 | 46.553 | 93.385 | 8.329 | 1.00 | 13.87 | 6 |
| ATOM | 4549 | CD1 | ILE | C | 129 | 47.506 | 94.531 | 8.013 | 1.00 | 14.82 | 6 |
| ATOM | 4550 | C | ILE | C | 129 | 46.917 | 89.588 | 7.843 | 1.00 | 52.95 | 6 |
| ATOM | 4551 | O | ILE | C | 129 | 47.298 | 89.107 | 8.906 | 1.00 | 52.12 | 8 |
| ATOM | 4552 | N | ASN | C | 130 | 47.023 | 88.967 | 6.676 | 1.00 | 33.54 | 7 |
| ATOM | 4553 | CA | ASN | C | 130 | 47.596 | 87.641 | 6.540 | 1.00 | 34.43 | 6 |
| ATOM | 4554 | CB | ASN | C | 130 | 48.944 | 87.552 | 7.254 | 1.00 | 65.84 | 6 |
| ATOM | 4555 | CG | ASN | C | 130 | 49.953 | 88.539 | 6.706 | 1.00 | 66.87 | 6 |
| ATOM | 4556 | OD1 | ASN | C | 130 | 50.326 | 88.488 | 5.531 | 1.00 | 66.73 | 8 |
| ATOM | 4557 | ND2 | ASN | C | 130 | 50.396 | 89.453 | 7.557 | 1.00 | 66.85 | 7 |
| ATOM | 4558 | C | ASN | C | 130 | 46.640 | 86.613 | 7.098 | 1.00 | 34.18 | 6 |
| ATOM | 4559 | O | ASN | C | 130 | 47.036 | 85.501 | 7.438 | 1.00 | 35.03 | 8 |
| ATOM | 4560 | N | GLY | C | 131 | 45.373 | 86.992 | 7.191 | 1.00 | 27.94 | 7 |
| ATOM | 4561 | CA | GLY | C | 131 | 44.376 | 86.071 | 7.698 | 1.00 | 26.19 | 6 |
| ATOM | 4562 | C | GLY | C | 131 | 43.759 | 86.491 | 9.008 | 1.00 | 24.96 | 6 |
| ATOM | 4563 | O | GLY | C | 131 | 42.551 | 86.411 | 9.164 | 1.00 | 23.55 | 8 |
| ATOM | 4564 | N | ALA | C | 132 | 44.595 | 86.938 | 9.938 | 1.00 | 40.17 | 7 |
| ATOM | 4565 | CA | ALA | C | 132 | 44.150 | 87.381 | 11.256 | 1.00 | 40.65 | 6 |
| ATOM | 4566 | CB | ALA | C | 132 | 45.280 | 87.214 | 12.257 | 1.00 | 77.38 | 6 |
| ATOM | 4567 | C | ALA | C | 132 | 43.641 | 88.832 | 11.283 | 1.00 | 40.65 | 6 |
| ATOM | 4568 | O | ALA | C | 132 | 43.964 | 89.640 | 10.407 | 1.00 | 41.21 | 8 |
| ATOM | 4569 | N | ASP | C | 133 | 42.843 | 89.149 | 12.302 | 1.00 | 15.55 | 7 |
| ATOM | 4570 | CA | ASP | C | 133 | 42.277 | 90.478 | 12.448 | 1.00 | 14.38 | 6 |
| ATOM | 4571 | CB | ASP | C | 133 | 40.892 | 90.404 | 13.073 | 1.00 | 64.17 | 6 |
| ATOM | 4572 | CG | ASP | C | 133 | 39.938 | 89.573 | 12.266 | 1.00 | 67.59 | 6 |
| ATOM | 4573 | OD1 | ASP | C | 133 | 39.457 | 90.068 | 11.226 | 1.00 | 70.82 | 8 |
| ATOM | 4574 | OD2 | ASP | C | 133 | 39.675 | 88.420 | 12.673 | 1.00 | 69.26 | 8 |
| ATOM | 4575 | C | ASP | C | 133 | 43.149 | 91.312 | 13.334 | 1.00 | 13.87 | 6 |
| ATOM | 4576 | O | ASP | C | 133 | 43.591 | 90.867 | 14.393 | 1.00 | 13.87 | 8 |
| ATOM | 4577 | N | ARG | C | 134 | 43.386 | 92.540 | 12.901 | 1.00 | 31.25 | 7 |
| ATOM | 4578 | CA | ARG | C | 134 | 44.204 | 93.457 | 13.677 | 1.00 | 30.63 | 6 |
| ATOM | 4579 | CB | ARG | C | 134 | 45.627 | 93.572 | 13.118 | 1.00 | 47.57 | 6 |
| ATOM | 4580 | CG | ARG | C | 134 | 46.194 | 92.313 | 12.479 | 1.00 | 51.45 | 6 |
| ATOM | 4581 | CD | ARG | C | 134 | 47.698 | 92.335 | 12.518 | 1.00 | 52.73 | 6 |
| ATOM | 4582 | NE | ARG | C | 134 | 48.169 | 91.668 | 13.716 | 1.00 | 54.72 | 7 |
| ATOM | 4583 | CZ | ARG | C | 134 | 49.360 | 91.878 | 14.247 | 1.00 | 57.60 | 6 |
| ATOM | 4584 | NH1 | ARG | C | 134 | 50.173 | 92.749 | 13.668 | 1.00 | 58.08 | 7 |
| ATOM | 4585 | NH2 | ARG | C | 134 | 49.741 | 91.211 | 15.332 | 1.00 | 60.44 | 7 |
| ATOM | 4586 | C | ARG | C | 134 | 43.570 | 94.828 | 13.643 | 1.00 | 28.37 | 6 |
| ATOM | 4587 | O | ARG | C | 134 | 42.817 | 95.160 | 12.719 | 1.00 | 28.20 | 8 |
| ATOM | 4588 | N | VAL | C | 135 | 43.906 | 95.621 | 14.656 | 1.00 | 23.37 | 7 |
| ATOM | 4589 | CA | VAL | C | 135 | 43.402 | 96.976 | 14.800 | 1.00 | 20.65 | 6 |
| ATOM | 4590 | CB | VAL | C | 135 | 42.484 | 97.063 | 16.037 | 1.00 | 13.87 | 6 |
| ATOM | 4591 | CG1 | VAL | C | 135 | 41.236 | 97.848 | 15.700 | 1.00 | 13.87 | 6 |
| ATOM | 4592 | CG2 | VAL | C | 135 | 42.127 | 95.653 | 16.528 | 1.00 | 13.87 | 6 |
| ATOM | 4593 | C | VAL | C | 135 | 44.584 | 97.953 | 14.953 | 1.00 | 20.23 | 6 |
| ATOM | 4594 | O | VAL | C | 135 | 45.553 | 97.664 | 15.641 | 1.00 | 19.42 | 8 |
| ATOM | 4595 | N | ILE | C | 136 | 44.509 | 99.099 | 14.294 | 1.00 | 17.38 | 7 |
| ATOM | 4596 | CA | ILE | C | 136 | 45.566 | 100.095 | 14.392 | 1.00 | 19.94 | 6 |
| ATOM | 4597 | CB | ILE | C | 136 | 45.684 | 100.894 | 13.088 | 1.00 | 37.79 | 6 |
| ATOM | 4598 | CG2 | ILE | C | 136 | 46.695 | 102.030 | 13.252 | 1.00 | 39.14 | 6 |
| ATOM | 4599 | CG1 | ILE | C | 136 | 46.090 | 99.953 | 11.957 | 1.00 | 39.94 | 6 |
| ATOM | 4600 | CD1 | ILE | C | 136 | 47.387 | 99.226 | 12.220 | 1.00 | 42.63 | 6 |
| ATOM | 4601 | C | ILE | C | 136 | 45.308 | 101.071 | 15.548 | 1.00 | 20.26 | 6 |
| ATOM | 4602 | O | ILE | C | 136 | 44.535 | 102.020 | 15.407 | 1.00 | 18.99 | 8 |
| ATOM | 4603 | N | VAL | C | 137 | 45.964 | 100.824 | 16.682 | 1.00 | 25.21 | 7 |
| ATOM | 4604 | CA | VAL | C | 137 | 45.827 | 101.646 | 17.888 | 1.00 | 25.11 | 6 |
| ATOM | 4605 | CB | VAL | C | 137 | 46.507 | 100.985 | 19.088 | 1.00 | 16.86 | 6 |
| ATOM | 4606 | CG1 | VAL | C | 137 | 46.279 | 101.804 | 20.338 | 1.00 | 15.96 | 6 |
| ATOM | 4607 | CG2 | VAL | C | 137 | 45.983 | 99.583 | 19.255 | 1.00 | 16.90 | 6 |
| ATOM | 4608 | C | VAL | C | 137 | 46.454 | 103.003 | 17.684 | 1.00 | 25.80 | 6 |
| ATOM | 4609 | O | VAL | C | 137 | 47.591 | 103.111 | 17.244 | 1.00 | 26.80 | 8 |
| ATOM | 4610 | N | SER | C | 138 | 45.703 | 104.033 | 18.046 | 1.00 | 16.42 | 7 |
| ATOM | 4611 | CA | SER | C | 138 | 46.108 | 105.426 | 17.881 | 1.00 | 18.38 | 6 |
| ATOM | 4612 | CB | SER | C | 138 | 44.825 | 106.233 | 17.718 | 1.00 | 28.33 | 6 |
| ATOM | 4613 | OG | SER | C | 138 | 43.729 | 105.459 | 18.193 | 1.00 | 25.47 | 8 |
| ATOM | 4614 | C | SER | C | 138 | 46.982 | 106.007 | 18.998 | 1.00 | 20.62 | 6 |
| ATOM | 4615 | O | SER | C | 138 | 46.548 | 106.092 | 20.145 | 1.00 | 20.75 | 8 |
| ATOM | 4616 | N | GLN | C | 139 | 48.199 | 106.433 | 18.665 | 1.00 | 29.81 | 7 |
| ATOM | 4617 | CA | GLN | C | 139 | 49.080 | 106.970 | 19.698 | 1.00 | 33.75 | 6 |
| ATOM | 4618 | CB | GLN | C | 139 | 50.535 | 106.576 | 19.443 | 1.00 | 57.86 | 6 |
| ATOM | 4619 | CG | GLN | C | 139 | 51.270 | 107.377 | 18.388 | 1.00 | 59.42 | 6 |
| ATOM | 4620 | CD | GLN | C | 139 | 52.765 | 107.079 | 18.396 | 1.00 | 61.08 | 6 |
| ATOM | 4621 | OE1 | GLN | C | 139 | 53.183 | 105.925 | 18.295 | 1.00 | 60.50 | 8 |
| ATOM | 4622 | NE2 | GLN | C | 139 | 53.574 | 108.122 | 18.515 | 1.00 | 62.00 | 7 |
| ATOM | 4623 | C | GLN | C | 139 | 48.999 | 108.468 | 19.826 | 1.00 | 36.24 | 6 |
| ATOM | 4624 | O | GLN | C | 139 | 48.320 | 109.107 | 19.040 | 1.00 | 37.91 | 8 |
| ATOM | 4625 | N | ILE | C | 140 | 49.678 | 109.023 | 20.831 | 1.00 | 69.32 | 7 |
| ATOM | 4626 | CA | ILE | C | 140 | 49.696 | 110.472 | 21.049 | 1.00 | 71.94 | 6 |
| ATOM | 4627 | CB | ILE | C | 140 | 48.751 | 110.921 | 22.203 | 1.00 | 32.52 | 6 |
| ATOM | 4628 | CG2 | ILE | C | 140 | 47.328 | 110.632 | 21.853 | 1.00 | 32.85 | 6 |
| ATOM | 4629 | CG1 | ILE | C | 140 | 49.099 | 110.211 | 23.502 | 1.00 | 31.17 | 6 |
| ATOM | 4630 | CD1 | ILE | C | 140 | 48.294 | 110.720 | 24.656 | 1.00 | 28.81 | 6 |
| ATOM | 4631 | C | ILE | C | 140 | 51.088 | 111.003 | 21.379 | 1.00 | 74.89 | 6 |
| ATOM | 4632 | O | ILE | C | 140 | 51.830 | 110.392 | 22.151 | 1.00 | 76.12 | 8 |
| ATOM | 4633 | N | HIS | C | 141 | 51.437 | 112.144 | 20.785 | 1.00 | 41.40 | 7 |
| ATOM | 4634 | CA | HIS | C | 141 | 52.731 | 112.792 | 21.026 | 1.00 | 44.28 | 6 |
| ATOM | 4635 | CB | HIS | C | 141 | 53.683 | 112.585 | 19.846 | 1.00 | 100.71 | 6 |
| ATOM | 4636 | CG | HIS | C | 141 | 53.140 | 113.080 | 18.541 | 1.00 | 103.58 | 6 |
| ATOM | 4637 | CD2 | HIS | C | 141 | 53.213 | 114.296 | 17.949 | 1.00 | 105.25 | 6 |
| ATOM | 4638 | ND1 | HIS | C | 141 | 52.387 | 112.290 | 17.700 | 1.00 | 104.95 | 7 |
| ATOM | 4639 | CE1 | HIS | C | 141 | 52.019 | 112.997 | 16.647 | 1.00 | 105.35 | 6 |
| ATOM | 4640 | NE2 | HIS | C | 141 | 52.507 | 114.218 | 16.773 | 1.00 | 105.39 | 7 |
| ATOM | 4641 | C | HIS | C | 141 | 52.497 | 114.284 | 21.222 | 1.00 | 45.08 | 6 |
| ATOM | 4642 | O | HIS | C | 141 | 51.361 | 114.753 | 21.199 | 1.00 | 45.45 | 8 |
| ATOM | 4643 | N | ARG | C | 142 | 53.560 | 115.046 | 21.404 | 1.00 | 45.25 | 7 |
| ATOM | 4644 | CA | ARG | C | 142 | 53.350 | 116.465 | 21.603 | 1.00 | 46.96 | 6 |
| ATOM | 4645 | CB | ARG | C | 142 | 54.602 | 117.101 | 22.211 | 1.00 | 87.26 | 6 |
| ATOM | 4646 | CG | ARG | C | 142 | 54.293 | 118.353 | 23.024 | 1.00 | 88.28 | 6 |
| ATOM | 4647 | CD | ARG | C | 142 | 54.803 | 118.265 | 24.456 | 1.00 | 88.37 | 6 |
| ATOM | 4648 | NE | ARG | C | 142 | 56.001 | 119.070 | 24.671 | 1.00 | 85.96 | 7 |
| ATOM | 4649 | CZ | ARG | C | 142 | 57.179 | 118.808 | 24.125 | 1.00 | 84.10 | 6 |
| ATOM | 4650 | NH1 | ARG | C | 142 | 57.320 | 117.756 | 23.334 | 1.00 | 81.90 | 7 |
| ATOM | 4651 | NH2 | ARG | C | 142 | 58.209 | 119.606 | 24.361 | 1.00 | 83.03 | 7 |
| ATOM | 4652 | C | ARG | C | 142 | 52.988 | 117.131 | 20.276 | 1.00 | 47.16 | 6 |
| ATOM | 4653 | O | ARG | C | 142 | 53.758 | 117.043 | 19.325 | 1.00 | 46.66 | 8 |
| ATOM | 4654 | N | SER | C | 143 | 51.825 | 117.788 | 20.207 | 1.00 | 24.06 | 7 |
| ATOM | 4655 | CA | SER | C | 143 | 51.377 | 118.446 | 18.966 | 1.00 | 24.67 | 6 |
| ATOM | 4656 | CB | SER | C | 143 | 50.112 | 119.266 | 19.219 | 1.00 | 137.30 | 6 |
| ATOM | 4657 | OG | SER | C | 143 | 50.380 | 120.379 | 20.052 | 1.00 | 140.68 | 8 |
| ATOM | 4658 | C | SER | C | 143 | 52.455 | 119.347 | 18.388 | 1.00 | 23.88 | 6 |
| ATOM | 4659 | O | SER | C | 143 | 52.917 | 120.257 | 19.040 | 1.00 | 23.01 | 8 |
| ATOM | 4660 | N | PRO | C | 144 | 52.861 | 119.102 | 17.145 | 1.00 | 54.84 | 7 |
| ATOM | 4661 | CD | PRO | C | 144 | 52.222 | 118.197 | 16.178 | 1.00 | 78.94 | 6 |
| ATOM | 4662 | CA | PRO | C | 144 | 53.898 | 119.904 | 16.500 | 1.00 | 56.24 | 6 |
| ATOM | 4663 | CB | PRO | C | 144 | 53.619 | 119.686 | 15.025 | 1.00 | 79.24 | 6 |
| ATOM | 4664 | CG | PRO | C | 144 | 53.168 | 118.272 | 15.006 | 1.00 | 79.94 | 6 |
| ATOM | 4665 | C | PRO | C | 144 | 53.867 | 121.377 | 16.886 | 1.00 | 57.78 | 6 |
| ATOM | 4666 | O | PRO | C | 144 | 52.795 | 121.989 | 16.943 | 1.00 | 58.39 | 8 |
| ATOM | 4667 | N | GLY | C | 145 | 55.043 | 121.943 | 17.150 | 1.00 | 56.07 | 7 |
| ATOM | 4668 | CA | GLY | C | 145 | 55.116 | 123.345 | 17.516 | 1.00 | 57.15 | 6 |
| ATOM | 4669 | C | GLY | C | 145 | 56.431 | 123.783 | 18.132 | 1.00 | 58.03 | 6 |
| ATOM | 4670 | O | GLY | C | 145 | 57.505 | 123.559 | 17.576 | 1.00 | 58.65 | 8 |
| ATOM | 4671 | N | VAL | C | 146 | 56.328 | 124.409 | 19.301 | 1.00 | 70.55 | 7 |
| ATOM | 4672 | CA | VAL | C | 146 | 57.469 | 124.934 | 20.054 | 1.00 | 71.73 | 6 |
| ATOM | 4673 | CB | VAL | C | 146 | 58.084 | 126.164 | 19.321 | 1.00 | 86.38 | 6 |
| ATOM | 4674 | CG1 | VAL | C | 146 | 58.807 | 127.069 | 20.304 | 1.00 | 87.82 | 6 |
| ATOM | 4675 | CG2 | VAL | C | 146 | 59.045 | 125.699 | 18.241 | 1.00 | 87.33 | 6 |
| ATOM | 4676 | C | VAL | C | 146 | 56.920 | 125.377 | 21.411 | 1.00 | 71.72 | 6 |
| ATOM | 4677 | O | VAL | C | 146 | 55.961 | 126.139 | 21.457 | 1.00 | 71.68 | 8 |
| ATOM | 4678 | N | TYR | C | 147 | 57.503 | 124.911 | 22.512 | 1.00 | 117.29 | 7 |
| ATOM | 4679 | CA | TYR | C | 147 | 56.984 | 125.318 | 23.814 | 1.00 | 117.99 | 6 |
| ATOM | 4680 | CB | TYR | C | 147 | 56.107 | 124.229 | 24.423 | 1.00 | 56.96 | 6 |
| ATOM | 4681 | CG | TYR | C | 147 | 55.592 | 123.190 | 23.463 | 1.00 | 55.38 | 6 |
| ATOM | 4682 | CD1 | TYR | C | 147 | 56.452 | 122.268 | 22.880 | 1.00 | 54.18 | 6 |
| ATOM | 4683 | CE1 | TYR | C | 147 | 55.963 | 121.274 | 22.056 | 1.00 | 53.00 | 6 |
| ATOM | 4684 | CD2 | TYR | C | 147 | 54.230 | 123.090 | 23.186 | 1.00 | 55.16 | 6 |
| ATOM | 4685 | CE2 | TYR | C | 147 | 53.739 | 122.106 | 22.365 | 1.00 | 53.62 | 6 |
| ATOM | 4686 | CZ | TYR | C | 147 | 54.604 | 121.201 | 21.807 | 1.00 | 52.84 | 6 |
| ATOM | 4687 | OH | TYR | C | 147 | 54.104 | 120.196 | 21.022 | 1.00 | 53.09 | 8 |
| ATOM | 4688 | C | TYR | C | 147 | 58.020 | 125.703 | 24.856 | 1.00 | 119.45 | 6 |
| ATOM | 4689 | O | TYR | C | 147 | 57.672 | 126.267 | 25.890 | 1.00 | 121.23 | 8 |
| ATOM | 4690 | N | PHE | C | 148 | 59.282 | 125.385 | 24.599 | 1.00 | 127.73 | 7 |
| ATOM | 4691 | CA | PHE | C | 148 | 60.356 | 125.701 | 25.534 | 1.00 | 127.40 | 6 |
| ATOM | 4692 | CB | PHE | C | 148 | 60.178 | 127.120 | 26.072 | 1.00 | 72.62 | 6 |
| ATOM | 4693 | CG | PHE | C | 148 | 60.496 | 128.170 | 25.051 | 1.00 | 72.89 | 6 |
| ATOM | 4694 | CD1 | PHE | C | 148 | 59.873 | 128.150 | 23.813 | 1.00 | 72.38 | 6 |
| ATOM | 4695 | CD2 | PHE | C | 148 | 61.487 | 129.109 | 25.281 | 1.00 | 73.32 | 6 |
| ATOM | 4696 | CE1 | PHE | C | 148 | 60.239 | 129.032 | 22.820 | 1.00 | 72.68 | 6 |
| ATOM | 4697 | CE2 | PHE | C | 148 | 61.859 | 129.995 | 24.290 | 1.00 | 73.89 | 6 |
| ATOM | 4698 | CZ | PHE | C | 148 | 61.234 | 129.956 | 23.056 | 1.00 | 73.37 | 6 |
| ATOM | 4699 | C | PHE | C | 148 | 60.465 | 124.672 | 26.653 | 1.00 | 127.51 | 6 |
| ATOM | 4700 | O | PHE | C | 148 | 60.867 | 123.547 | 26.365 | 1.00 | 129.30 | 8 |
| ATOM | 4701 | N | THR | C | 149 | 60.129 | 125.027 | 27.899 | 1.00 | 41.88 | 7 |
| ATOM | 4702 | CA | THR | C | 149 | 60.218 | 124.066 | 29.020 | 1.00 | 40.35 | 6 |
| ATOM | 4703 | CB | THR | C | 149 | 60.313 | 122.622 | 28.508 | 1.00 | 45.51 | 6 |
| ATOM | 4704 | OG1 | THR | C | 149 | 59.144 | 122.319 | 27.751 | 1.00 | 45.70 | 8 |
| ATOM | 4705 | CG2 | THR | C | 149 | 60.481 | 121.638 | 29.638 | 1.00 | 45.67 | 6 |
| ATOM | 4706 | C | THR | C | 149 | 61.430 | 124.263 | 29.932 | 1.00 | 39.60 | 6 |
| ATOM | 4707 | O | THR | C | 149 | 62.506 | 124.636 | 29.468 | 1.00 | 39.78 | 8 |
| ATOM | 4708 | N | PRO | C | 150 | 61.274 | 123.994 | 31.244 | 1.00 | 43.39 | 7 |
| ATOM | 4709 | CD | PRO | C | 150 | 60.009 | 123.690 | 31.934 | 1.00 | 74.13 | 6 |
| ATOM | 4710 | CA | PRO | C | 150 | 62.356 | 124.134 | 32.226 | 1.00 | 42.51 | 6 |
| ATOM | 4711 | CB | PRO | C | 150 | 61.624 | 124.041 | 33.559 | 1.00 | 73.28 | 6 |
| ATOM | 4712 | CG | PRO | C | 150 | 60.504 | 123.118 | 33.242 | 1.00 | 74.47 | 6 |
| ATOM | 4713 | C | PRO | C | 150 | 63.445 | 123.072 | 32.100 | 1.00 | 41.93 | 6 |
| ATOM | 4714 | O | PRO | C | 150 | 63.175 | 121.882 | 32.204 | 1.00 | 41.14 | 8 |
| ATOM | 4715 | N | ASP | C | 151 | 64.675 | 123.523 | 31.888 | 1.00 | 60.62 | 7 |
| ATOM | 4716 | CA | ASP | C | 151 | 65.818 | 122.636 | 31.750 | 1.00 | 61.64 | 6 |
| ATOM | 4717 | CB | ASP | C | 151 | 67.095 | 123.459 | 31.661 | 1.00 | 82.47 | 6 |
| ATOM | 4718 | CG | ASP | C | 151 | 68.284 | 122.636 | 31.246 | 1.00 | 83.64 | 6 |
| ATOM | 4719 | OD1 | ASP | C | 151 | 68.498 | 121.571 | 31.861 | 1.00 | 85.08 | 8 |
| ATOM | 4720 | OD2 | ASP | C | 151 | 69.005 | 123.054 | 30.310 | 1.00 | 83.86 | 8 |
| ATOM | 4721 | C | ASP | C | 151 | 65.884 | 121.736 | 32.970 | 1.00 | 62.64 | 6 |
| ATOM | 4722 | O | ASP | C | 151 | 66.220 | 122.190 | 34.060 | 1.00 | 62.88 | 8 |
| ATOM | 4723 | N | PRO | C | 152 | 65.584 | 120.441 | 32.801 | 1.00 | 53.51 | 7 |
| ATOM | 4724 | CD | PRO | C | 152 | 65.413 | 119.745 | 31.515 | 1.00 | 152.73 | 6 |
| ATOM | 4725 | CA | PRO | C | 152 | 65.605 | 119.475 | 33.907 | 1.00 | 53.78 | 6 |
| ATOM | 4726 | CB | PRO | C | 152 | 65.331 | 118.146 | 33.205 | 1.00 | 153.68 | 6 |
| ATOM | 4727 | CG | PRO | C | 152 | 65.910 | 118.364 | 31.837 | 1.00 | 153.85 | 6 |
| ATOM | 4728 | C | PRO | C | 152 | 66.890 | 119.445 | 34.742 | 1.00 | 53.62 | 6 |
| ATOM | 4729 | O | PRO | C | 152 | 66.836 | 119.280 | 35.965 | 1.00 | 52.96 | 8 |
| ATOM | 4730 | N | ALA | C | 153 | 68.034 | 119.590 | 34.077 | 1.00 | 112.64 | 7 |
| ATOM | 4731 | CA | ALA | C | 153 | 69.326 | 119.588 | 34.754 | 1.00 | 113.39 | 6 |
| ATOM | 4732 | CB | ALA | C | 153 | 70.404 | 120.125 | 33.817 | 1.00 | 30.18 | 6 |
| ATOM | 4733 | C | ALA | C | 153 | 69.232 | 120.458 | 36.002 | 1.00 | 113.68 | 6 |
| ATOM | 4734 | O | ALA | C | 153 | 68.842 | 119.992 | 37.075 | 1.00 | 113.62 | 8 |
| ATOM | 4735 | N | ARG | C | 154 | 69.600 | 121.724 | 35.854 | 1.00 | 60.59 | 7 |
| ATOM | 4736 | CA | ARG | C | 154 | 69.532 | 122.675 | 36.953 | 1.00 | 60.51 | 6 |
| ATOM | 4737 | CB | ARG | C | 154 | 70.924 | 122.944 | 37.515 | 1.00 | 102.11 | 6 |
| ATOM | 4738 | CG | ARG | C | 154 | 71.529 | 121.762 | 38.255 | 1.00 | 103.38 | 6 |
| ATOM | 4739 | CD | ARG | C | 154 | 70.707 | 121.387 | 39.481 | 1.00 | 105.00 | 6 |
| ATOM | 4740 | NE | ARG | C | 154 | 71.391 | 120.388 | 40.300 | 1.00 | 106.91 | 7 |
| ATOM | 4741 | CZ | ARG | C | 154 | 70.934 | 119.922 | 41.459 | 1.00 | 107.11 | 6 |
| ATOM | 4742 | NH1 | ARG | C | 154 | 69.782 | 120.362 | 41.946 | 1.00 | 107.07 | 7 |
| ATOM | 4743 | NH2 | ARG | C | 154 | 71.634 | 119.018 | 42.133 | 1.00 | 106.92 | 7 |
| ATOM | 4744 | C | ARG | C | 154 | 68.934 | 123.945 | 36.367 | 1.00 | 60.26 | 6 |
| ATOM | 4745 | O | ARG | C | 154 | 69.385 | 124.409 | 35.317 | 1.00 | 60.90 | 8 |
| ATOM | 4746 | N | PRO | C | 155 | 67.921 | 124.525 | 37.046 | 1.00 | 123.78 | 7 |
| ATOM | 4747 | CD | PRO | C | 155 | 67.773 | 124.324 | 38.492 | 1.00 | 72.44 | 6 |
| ATOM | 4748 | CA | PRO | C | 155 | 67.197 | 125.742 | 36.654 | 1.00 | 123.95 | 6 |
| ATOM | 4749 | CB | PRO | C | 155 | 67.106 | 126.545 | 37.964 | 1.00 | 72.35 | 6 |
| ATOM | 4750 | CG | PRO | C | 155 | 67.916 | 125.733 | 38.984 | 1.00 | 72.06 | 6 |
| ATOM | 4751 | C | PRO | C | 155 | 67.791 | 126.542 | 35.500 | 1.00 | 123.67 | 6 |
| ATOM | 4752 | O | PRO | C | 155 | 68.825 | 127.200 | 35.637 | 1.00 | 123.37 | 8 |
| ATOM | 4753 | N | GLY | C | 156 | 67.118 | 126.474 | 34.356 | 1.00 | 79.22 | 7 |
| ATOM | 4754 | CA | GLY | C | 156 | 67.586 | 127.186 | 33.188 | 1.00 | 78.18 | 6 |
| ATOM | 4755 | C | GLY | C | 156 | 66.950 | 126.674 | 31.918 | 1.00 | 78.05 | 6 |
| ATOM | 4756 | O | GLY | C | 156 | 67.636 | 126.094 | 31.082 | 1.00 | 78.51 | 8 |
| ATOM | 4757 | N | ARG | C | 157 | 65.645 | 126.895 | 31.778 | 1.00 | 53.95 | 7 |
| ATOM | 4758 | CA | ARG | C | 157 | 64.886 | 126.477 | 30.597 | 1.00 | 53.98 | 6 |
| ATOM | 4759 | CB | ARG | C | 157 | 63.700 | 127.418 | 30.360 | 1.00 | 62.64 | 6 |
| ATOM | 4760 | CG | ARG | C | 157 | 63.671 | 128.658 | 31.234 | 1.00 | 62.58 | 6 |
| ATOM | 4761 | CD | ARG | C | 157 | 62.903 | 128.383 | 32.518 | 1.00 | 62.90 | 6 |
| ATOM | 4762 | NE | ARG | C | 157 | 62.815 | 129.558 | 33.379 | 1.00 | 62.14 | 7 |
| ATOM | 4763 | CZ | ARG | C | 157 | 62.214 | 129.572 | 34.565 | 1.00 | 61.89 | 6 |
| ATOM | 4764 | NH1 | ARG | C | 157 | 61.643 | 128.470 | 35.040 | 1.00 | 61.21 | 7 |
| ATOM | 4765 | NH2 | ARG | C | 157 | 62.191 | 130.688 | 35.282 | 1.00 | 61.30 | 7 |
| ATOM | 4766 | C | ARG | C | 157 | 65.648 | 126.362 | 29.269 | 1.00 | 54.44 | 6 |
| ATOM | 4767 | O | ARG | C | 157 | 66.756 | 126.893 | 29.096 | 1.00 | 54.30 | 8 |
| ATOM | 4768 | N | TYR | C | 158 | 65.030 | 125.655 | 28.327 | 1.00 | 121.76 | 7 |
| ATOM | 4769 | CA | TYR | C | 158 | 65.591 | 125.473 | 26.994 | 1.00 | 122.22 | 6 |
| ATOM | 4770 | CB | TYR | C | 158 | 66.222 | 124.088 | 26.830 | 1.00 | 123.23 | 6 |
| ATOM | 4771 | CG | TYR | C | 158 | 65.461 | 122.969 | 27.486 | 1.00 | 124.02 | 6 |
| ATOM | 4772 | CD1 | TYR | C | 158 | 64.074 | 122.998 | 27.575 | 1.00 | 124.12 | 6 |
| ATOM | 4773 | CE1 | TYR | C | 158 | 63.373 | 121.966 | 28.181 | 1.00 | 125.39 | 6 |
| ATOM | 4774 | CD2 | TYR | C | 158 | 66.133 | 121.874 | 28.016 | 1.00 | 125.08 | 6 |
| ATOM | 4775 | CE2 | TYR | C | 158 | 65.443 | 120.833 | 28.621 | 1.00 | 126.19 | 6 |
| ATOM | 4776 | CZ | TYR | C | 158 | 64.059 | 120.885 | 28.703 | 1.00 | 126.37 | 6 |
| ATOM | 4777 | OH | TYR | C | 158 | 63.365 | 119.860 | 29.310 | 1.00 | 126.79 | 8 |
| ATOM | 4778 | C | TYR | C | 158 | 64.490 | 125.649 | 25.967 | 1.00 | 121.49 | 6 |
| ATOM | 4779 | O | TYR | C | 158 | 63.373 | 126.041 | 26.300 | 1.00 | 121.07 | 8 |
| ATOM | 4780 | N | ILE | C | 159 | 64.807 | 125.356 | 24.714 | 1.00 | 29.94 | 7 |
| ATOM | 4781 | CA | ILE | C | 159 | 63.836 | 125.500 | 23.648 | 1.00 | 29.68 | 6 |
| ATOM | 4782 | CB | ILE | C | 159 | 64.154 | 126.715 | 22.764 | 1.00 | 68.63 | 6 |
| ATOM | 4783 | CG2 | ILE | C | 159 | 62.944 | 127.066 | 21.902 | 1.00 | 68.71 | 6 |
| ATOM | 4784 | CG1 | ILE | C | 159 | 64.518 | 127.908 | 23.642 | 1.00 | 69.14 | 6 |
| ATOM | 4785 | CD1 | ILE | C | 159 | 64.982 | 129.105 | 22.855 | 1.00 | 70.48 | 6 |
| ATOM | 4786 | C | ILE | C | 159 | 63.781 | 124.269 | 22.757 | 1.00 | 29.29 | 6 |
| ATOM | 4787 | O | ILE | C | 159 | 64.799 | 123.804 | 22.231 | 1.00 | 28.38 | 8 |
| ATOM | 4788 | N | ALA | C | 160 | 62.574 | 123.746 | 22.596 | 1.00 | 62.13 | 7 |
| ATOM | 4789 | CA | ALA | C | 160 | 62.358 | 122.584 | 21.765 | 1.00 | 62.42 | 6 |
| ATOM | 4790 | CB | ALA | C | 160 | 61.670 | 121.492 | 22.576 | 1.00 | 13.87 | 6 |
| ATOM | 4791 | C | ALA | C | 160 | 61.495 | 123.014 | 20.589 | 1.00 | 63.06 | 6 |
| ATOM | 4792 | O | ALA | C | 160 | 60.536 | 123.767 | 20.749 | 1.00 | 63.00 | 8 |
| ATOM | 4793 | N | SER | C | 161 | 61.847 | 122.554 | 19.399 | 1.00 | 52.02 | 7 |
| ATOM | 4794 | CA | SER | C | 161 | 61.082 | 122.897 | 18.215 | 1.00 | 53.53 | 6 |
| ATOM | 4795 | CB | SER | C | 161 | 61.958 | 123.713 | 17.264 | 1.00 | 35.62 | 6 |
| ATOM | 4796 | OG | SER | C | 161 | 61.206 | 124.209 | 16.171 | 1.00 | 33.38 | 8 |
| ATOM | 4797 | C | SER | C | 161 | 60.604 | 121.603 | 17.542 | 1.00 | 55.90 | 6 |
| ATOM | 4798 | O | SER | C | 161 | 61.330 | 121.009 | 16.744 | 1.00 | 56.86 | 8 |
| ATOM | 4799 | N | ILE | C | 162 | 59.389 | 121.167 | 17.876 | 1.00 | 66.20 | 7 |
| ATOM | 4800 | CA | ILE | C | 162 | 58.800 | 119.942 | 17.321 | 1.00 | 69.04 | 6 |
| ATOM | 4801 | CB | ILE | C | 162 | 57.438 | 119.631 | 18.001 | 1.00 | 110.64 | 6 |
| ATOM | 4802 | CG2 | ILE | C | 162 | 56.653 | 118.604 | 17.196 | 1.00 | 110.49 | 6 |
| ATOM | 4803 | CG1 | ILE | C | 162 | 57.666 | 119.075 | 19.408 | 1.00 | 112.67 | 6 |
| ATOM | 4804 | CD1 | ILE | C | 162 | 58.586 | 119.892 | 20.279 | 1.00 | 115.54 | 6 |
| ATOM | 4805 | C | ILE | C | 162 | 58.595 | 119.997 | 15.802 | 1.00 | 70.21 | 6 |
| ATOM | 4806 | O | ILE | C | 162 | 58.684 | 121.048 | 15.184 | 1.00 | 70.59 | 8 |
| ATOM | 4807 | N | ILE | C | 163 | 58.319 | 118.851 | 15.205 | 1.00 | 55.46 | 7 |
| ATOM | 4808 | CA | ILE | C | 163 | 58.113 | 118.776 | 13.779 | 1.00 | 58.28 | 6 |
| ATOM | 4809 | CB | ILE | C | 163 | 59.297 | 118.055 | 13.117 | 1.00 | 108.27 | 6 |
| ATOM | 4810 | CG2 | ILE | C | 163 | 59.951 | 118.969 | 12.104 | 1.00 | 109.24 | 6 |
| ATOM | 4811 | CG1 | ILE | C | 163 | 60.352 | 117.705 | 14.166 | 1.00 | 109.10 | 6 |
| ATOM | 4812 | CD1 | ILE | C | 163 | 59.941 | 116.607 | 15.120 | 1.00 | 109.92 | 6 |
| ATOM | 4813 | C | ILE | C | 163 | 56.794 | 118.036 | 13.537 | 1.00 | 60.36 | 6 |
| ATOM | 4814 | O | ILE | C | 163 | 56.397 | 117.203 | 14.353 | 1.00 | 60.87 | 8 |
| ATOM | 4815 | N | PRO | C | 164 | 56.116 | 118.314 | 12.400 | 1.00 | 170.37 | 7 |
| ATOM | 4816 | CD | PRO | C | 164 | 56.844 | 118.802 | 11.219 | 1.00 | 206.16 | 6 |
| ATOM | 4817 | CA | PRO | C | 164 | 54.830 | 117.739 | 11.970 | 1.00 | 172.36 | 6 |
| ATOM | 4818 | CB | PRO | C | 164 | 54.944 | 117.723 | 10.440 | 1.00 | 206.05 | 6 |
| ATOM | 4819 | CG | PRO | C | 164 | 56.420 | 117.813 | 10.180 | 1.00 | 206.20 | 6 |
| ATOM | 4820 | C | PRO | C | 164 | 54.465 | 116.377 | 12.542 | 1.00 | 172.85 | 6 |
| ATOM | 4821 | O | PRO | C | 164 | 53.383 | 116.198 | 13.099 | 1.00 | 173.08 | 8 |
| ATOM | 4822 | N | LEU | C | 165 | 55.363 | 115.417 | 12.375 | 1.00 | 84.53 | 7 |
| ATOM | 4823 | CA | LEU | C | 165 | 55.172 | 114.069 | 12.893 | 1.00 | 86.24 | 6 |
| ATOM | 4824 | CB | LEU | C | 165 | 54.306 | 113.221 | 11.951 | 1.00 | 183.33 | 6 |
| ATOM | 4825 | CG | LEU | C | 165 | 52.809 | 113.557 | 11.928 | 1.00 | 185.30 | 6 |
| ATOM | 4826 | CD1 | LEU | C | 165 | 52.093 | 112.712 | 10.891 | 1.00 | 186.01 | 6 |
| ATOM | 4827 | CD2 | LEU | C | 165 | 52.213 | 113.320 | 13.302 | 1.00 | 185.64 | 6 |
| ATOM | 4828 | C | LEU | C | 165 | 56.557 | 113.476 | 13.012 | 1.00 | 86.63 | 6 |
| ATOM | 4829 | O | LEU | C | 165 | 57.412 | 113.713 | 12.158 | 1.00 | 86.89 | 8 |
| ATOM | 4830 | N | PRO | C | 166 | 56.799 | 112.698 | 14.075 | 1.00 | 129.88 | 7 |
| ATOM | 4831 | CD | PRO | C | 166 | 55.740 | 112.149 | 14.942 | 1.00 | 63.26 | 6 |
| ATOM | 4832 | CA | PRO | C | 166 | 58.087 | 112.049 | 14.348 | 1.00 | 130.73 | 6 |
| ATOM | 4833 | CB | PRO | C | 166 | 57.676 | 110.812 | 15.132 | 1.00 | 63.82 | 6 |
| ATOM | 4834 | CG | PRO | C | 166 | 56.530 | 111.347 | 15.955 | 1.00 | 63.96 | 6 |
| ATOM | 4835 | C | PRO | C | 166 | 58.897 | 111.725 | 13.091 | 1.00 | 131.07 | 6 |
| ATOM | 4836 | O | PRO | C | 166 | 59.954 | 112.310 | 12.855 | 1.00 | 131.68 | 8 |
| ATOM | 4837 | N | LYS | C | 167 | 58.399 | 110.795 | 12.285 | 1.00 | 154.09 | 7 |
| ATOM | 4838 | CA | LYS | C | 167 | 59.085 | 110.433 | 11.057 | 1.00 | 154.27 | 6 |
| ATOM | 4839 | CB | LYS | C | 167 | 58.579 | 109.081 | 10.544 | 1.00 | 100.00 | 6 |
| ATOM | 4840 | CG | LYS | C | 167 | 58.792 | 107.916 | 11.512 | 1.00 | 101.09 | 6 |
| ATOM | 4841 | CD | LYS | C | 167 | 58.184 | 106.614 | 10.967 | 1.00 | 101.62 | 6 |
| ATOM | 4842 | CE | LYS | C | 167 | 58.275 | 105.457 | 11.967 | 1.00 | 101.62 | 6 |
| ATOM | 4843 | NZ | LYS | C | 167 | 57.599 | 104.220 | 11.477 | 1.00 | 102.16 | 7 |
| ATOM | 4844 | C | LYS | C | 167 | 58.834 | 111.521 | 10.015 | 1.00 | 153.74 | 6 |
| ATOM | 4845 | O | LYS | C | 167 | 58.104 | 111.312 | 9.046 | 1.00 | 154.66 | 8 |
| ATOM | 4846 | N | ARG | C | 168 | 59.438 | 112.687 | 10.237 | 1.00 | 208.87 | 7 |
| ATOM | 4847 | CA | ARG | C | 168 | 59.309 | 113.829 | 9.335 | 1.00 | 208.87 | 6 |
| ATOM | 4848 | CB | ARG | C | 168 | 57.868 | 113.965 | 8.836 | 1.00 | 146.96 | 6 |
| ATOM | 4849 | CG | ARG | C | 168 | 57.609 | 115.305 | 8.191 | 1.00 | 149.03 | 6 |
| ATOM | 4850 | CD | ARG | C | 168 | 56.342 | 115.325 | 7.376 | 1.00 | 150.54 | 6 |
| ATOM | 4851 | NE | ARG | C | 168 | 56.202 | 116.607 | 6.692 | 1.00 | 151.71 | 7 |
| ATOM | 4852 | CZ | ARG | C | 168 | 55.323 | 116.851 | 5.727 | 1.00 | 153.30 | 6 |
| ATOM | 4853 | NH1 | ARG | C | 168 | 54.495 | 115.898 | 5.325 | 1.00 | 155.10 | 7 |
| ATOM | 4854 | NH2 | ARG | C | 168 | 55.272 | 118.049 | 5.161 | 1.00 | 153.41 | 7 |
| ATOM | 4855 | C | ARG | C | 168 | 59.736 | 115.157 | 9.967 | 1.00 | 208.87 | 6 |
| ATOM | 4856 | O | ARG | C | 168 | 59.030 | 115.699 | 10.818 | 1.00 | 208.87 | 8 |
| ATOM | 4857 | N | GLY | C | 169 | 60.880 | 115.685 | 9.534 | 1.00 | 101.34 | 7 |
| ATOM | 4858 | CA | GLY | C | 169 | 61.369 | 116.945 | 10.067 | 1.00 | 96.06 | 6 |
| ATOM | 4859 | C | GLY | C | 169 | 62.264 | 116.774 | 11.280 | 1.00 | 92.86 | 6 |
| ATOM | 4860 | O | GLY | C | 169 | 62.176 | 115.768 | 11.978 | 1.00 | 93.47 | 8 |
| ATOM | 4861 | N | PRO | C | 170 | 63.155 | 117.733 | 11.549 | 1.00 | 79.40 | 7 |
| ATOM | 4862 | CD | PRO | C | 170 | 63.788 | 118.589 | 10.539 | 1.00 | 58.08 | 6 |
| ATOM | 4863 | CA | PRO | C | 170 | 64.032 | 117.597 | 12.716 | 1.00 | 75.94 | 6 |
| ATOM | 4864 | CB | PRO | C | 170 | 65.343 | 118.234 | 12.253 | 1.00 | 55.31 | 6 |
| ATOM | 4865 | CG | PRO | C | 170 | 65.232 | 118.248 | 10.746 | 1.00 | 55.85 | 6 |
| ATOM | 4866 | C | PRO | C | 170 | 63.506 | 118.257 | 13.990 | 1.00 | 73.58 | 6 |
| ATOM | 4867 | O | PRO | C | 170 | 62.975 | 119.372 | 13.972 | 1.00 | 73.71 | 8 |
| ATOM | 4868 | N | TRP | C | 171 | 63.667 | 117.536 | 15.091 | 1.00 | 62.07 | 7 |
| ATOM | 4869 | CA | TRP | C | 171 | 63.265 | 117.978 | 16.415 | 1.00 | 58.42 | 6 |
| ATOM | 4870 | CB | TRP | C | 171 | 62.937 | 116.743 | 17.265 | 1.00 | 73.02 | 6 |
| ATOM | 4871 | CG | TRP | C | 171 | 62.826 | 116.966 | 18.741 | 1.00 | 71.24 | 6 |
| ATOM | 4872 | CD2 | TRP | C | 171 | 61.630 | 116.935 | 19.519 | 1.00 | 70.57 | 6 |
| ATOM | 4873 | CE2 | TRP | C | 171 | 61.996 | 117.177 | 20.858 | 1.00 | 69.79 | 6 |
| ATOM | 4874 | CE3 | TRP | C | 171 | 60.282 | 116.726 | 19.214 | 1.00 | 69.74 | 6 |
| ATOM | 4875 | CD1 | TRP | C | 171 | 63.844 | 117.218 | 19.616 | 1.00 | 70.80 | 6 |
| ATOM | 4876 | NE1 | TRP | C | 171 | 63.353 | 117.345 | 20.891 | 1.00 | 70.14 | 7 |
| ATOM | 4877 | CZ2 | TRP | C | 171 | 61.063 | 117.216 | 21.886 | 1.00 | 68.62 | 6 |
| ATOM | 4878 | CZ3 | TRP | C | 171 | 59.360 | 116.762 | 20.236 | 1.00 | 68.21 | 6 |
| ATOM | 4879 | CH2 | TRP | C | 171 | 59.752 | 117.007 | 21.556 | 1.00 | 68.40 | 6 |
| ATOM | 4880 | C | TRP | C | 171 | 64.474 | 118.728 | 16.964 | 1.00 | 57.22 | 6 |
| ATOM | 4881 | O | TRP | C | 171 | 65.439 | 118.116 | 17.436 | 1.00 | 57.05 | 8 |
| ATOM | 4882 | N | ILE | C | 172 | 64.422 | 120.054 | 16.869 | 1.00 | 46.00 | 7 |
| ATOM | 4883 | CA | ILE | C | 172 | 65.502 | 120.922 | 17.330 | 1.00 | 43.86 | 6 |
| ATOM | 4884 | CB | ILE | C | 172 | 65.619 | 122.181 | 16.417 | 1.00 | 53.44 | 6 |
| ATOM | 4885 | CG2 | ILE | C | 172 | 66.528 | 123.230 | 17.047 | 1.00 | 52.58 | 6 |
| ATOM | 4886 | CG1 | ILE | C | 172 | 66.152 | 121.776 | 15.044 | 1.00 | 53.18 | 6 |
| ATOM | 4887 | CD1 | ILE | C | 172 | 66.485 | 122.952 | 14.143 | 1.00 | 53.90 | 6 |
| ATOM | 4888 | C | ILE | C | 172 | 65.339 | 121.382 | 18.777 | 1.00 | 43.80 | 6 |
| ATOM | 4889 | O | ILE | C | 172 | 64.232 | 121.420 | 19.312 | 1.00 | 43.38 | 8 |
| ATOM | 4890 | N | ASP | C | 173 | 66.456 | 121.716 | 19.410 | 1.00 | 27.91 | 7 |
| ATOM | 4891 | CA | ASP | C | 173 | 66.447 | 122.216 | 20.773 | 1.00 | 28.66 | 6 |
| ATOM | 4892 | CB | ASP | C | 173 | 66.740 | 121.095 | 21.765 | 1.00 | 75.82 | 6 |
| ATOM | 4893 | CG | ASP | C | 173 | 65.505 | 120.346 | 22.177 | 1.00 | 77.50 | 6 |
| ATOM | 4894 | OD1 | ASP | C | 173 | 64.542 | 120.998 | 22.616 | 1.00 | 79.36 | 8 |
| ATOM | 4895 | OD2 | ASP | C | 173 | 65.498 | 119.105 | 22.074 | 1.00 | 78.29 | 8 |
| ATOM | 4896 | C | ASP | C | 173 | 67.500 | 123.312 | 20.918 | 1.00 | 29.71 | 6 |
| ATOM | 4897 | O | ASP | C | 173 | 68.668 | 123.104 | 20.580 | 1.00 | 29.08 | 8 |
| ATOM | 4898 | N | LEU | C | 174 | 67.092 | 124.485 | 21.393 | 1.00 | 51.31 | 7 |
| ATOM | 4899 | CA | LEU | C | 174 | 68.032 | 125.587 | 21.590 | 1.00 | 52.02 | 6 |
| ATOM | 4900 | CB | LEU | C | 174 | 67.456 | 126.917 | 21.099 | 1.00 | 30.46 | 6 |
| ATOM | 4901 | CG | LEU | C | 174 | 66.724 | 127.041 | 19.762 | 1.00 | 27.68 | 6 |
| ATOM | 4902 | CD1 | LEU | C | 174 | 66.242 | 128.477 | 19.614 | 1.00 | 27.71 | 6 |
| ATOM | 4903 | CD2 | LEU | C | 174 | 67.632 | 126.655 | 18.604 | 1.00 | 27.25 | 6 |
| ATOM | 4904 | C | LEU | C | 174 | 68.232 | 125.674 | 23.089 | 1.00 | 53.81 | 6 |
| ATOM | 4905 | O | LEU | C | 174 | 67.301 | 126.000 | 23.820 | 1.00 | 52.87 | 8 |
| ATOM | 4906 | N | GLU | C | 175 | 69.440 | 125.393 | 23.554 | 1.00 | 91.87 | 7 |
| ATOM | 4907 | CA | GLU | C | 175 | 69.693 | 125.428 | 24.985 | 1.00 | 95.65 | 6 |
| ATOM | 4908 | CB | GLU | C | 175 | 70.099 | 124.031 | 25.465 | 1.00 | 184.07 | 6 |
| ATOM | 4909 | CG | GLU | C | 175 | 70.397 | 123.942 | 26.955 | 1.00 | 187.25 | 6 |
| ATOM | 4910 | CD | GLU | C | 175 | 70.953 | 122.590 | 27.358 | 1.00 | 189.17 | 6 |
| ATOM | 4911 | OE1 | GLU | C | 175 | 72.020 | 122.204 | 26.832 | 1.00 | 189.79 | 8 |
| ATOM | 4912 | OE2 | GLU | C | 175 | 70.325 | 121.912 | 28.200 | 1.00 | 190.45 | 8 |
| ATOM | 4913 | C | GLU | C | 175 | 70.750 | 126.434 | 25.419 | 1.00 | 97.00 | 6 |
| ATOM | 4914 | O | GLU | C | 175 | 71.828 | 126.502 | 24.831 | 1.00 | 97.41 | 8 |
| ATOM | 4915 | N | VAL | C | 176 | 70.425 | 127.220 | 26.445 | 1.00 | 109.36 | 7 |
| ATOM | 4916 | CA | VAL | C | 176 | 71.362 | 128.192 | 27.008 | 1.00 | 111.86 | 6 |
| ATOM | 4917 | CB | VAL | C | 176 | 70.631 | 129.383 | 27.649 | 1.00 | 99.51 | 6 |
| ATOM | 4918 | CG1 | VAL | C | 176 | 70.510 | 130.519 | 26.653 | 1.00 | 99.77 | 6 |
| ATOM | 4919 | CG2 | VAL | C | 176 | 69.251 | 128.941 | 28.118 | 1.00 | 99.99 | 6 |
| ATOM | 4920 | C | VAL | C | 176 | 72.099 | 127.418 | 28.096 | 1.00 | 113.46 | 6 |
| ATOM | 4921 | O | VAL | C | 176 | 71.473 | 126.934 | 29.041 | 1.00 | 113.99 | 8 |
| ATOM | 4922 | N | GLU | C | 177 | 73.418 | 127.301 | 27.963 | 1.00 | 114.43 | 7 |
| ATOM | 4923 | CA | GLU | C | 177 | 74.213 | 126.533 | 28.916 | 1.00 | 115.97 | 6 |
| ATOM | 4924 | CB | GLU | C | 177 | 74.934 | 125.409 | 28.174 | 1.00 | 69.53 | 6 |
| ATOM | 4925 | CG | GLU | C | 177 | 75.954 | 125.905 | 27.171 | 1.00 | 69.55 | 6 |
| ATOM | 4926 | CD | GLU | C | 177 | 75.801 | 125.258 | 25.812 | 1.00 | 70.88 | 6 |
| ATOM | 4927 | OE1 | GLU | C | 177 | 74.720 | 125.413 | 25.211 | 1.00 | 72.04 | 8 |
| ATOM | 4928 | OE2 | GLU | C | 177 | 76.757 | 124.598 | 25.341 | 1.00 | 72.21 | 8 |
| ATOM | 4929 | C | GLU | C | 177 | 75.235 | 127.319 | 29.726 | 1.00 | 117.14 | 6 |
| ATOM | 4930 | O | GLU | C | 177 | 75.312 | 128.547 | 29.652 | 1.00 | 118.00 | 8 |
| ATOM | 4931 | N | ALA | C | 178 | 76.021 | 126.584 | 30.505 | 1.00 | 86.35 | 7 |
| ATOM | 4932 | CA | ALA | C | 178 | 77.059 | 127.180 | 31.330 | 1.00 | 86.34 | 6 |
| ATOM | 4933 | CB | ALA | C | 178 | 77.411 | 126.249 | 32.471 | 1.00 | 60.35 | 6 |
| ATOM | 4934 | C | ALA | C | 178 | 78.278 | 127.404 | 30.461 | 1.00 | 86.27 | 6 |
| ATOM | 4935 | O | ALA | C | 178 | 78.648 | 128.541 | 30.163 | 1.00 | 86.17 | 8 |
| ATOM | 4936 | N | SER | C | 179 | 78.888 | 126.297 | 30.051 | 1.00 | 124.62 | 7 |
| ATOM | 4937 | CA | SER | C | 179 | 80.078 | 126.325 | 29.215 | 1.00 | 124.26 | 6 |
| ATOM | 4938 | CB | SER | C | 179 | 81.081 | 125.283 | 29.720 | 1.00 | 83.67 | 6 |
| ATOM | 4939 | OG | SER | C | 179 | 80.429 | 124.068 | 30.043 | 1.00 | 83.11 | 8 |
| ATOM | 4940 | C | SER | C | 179 | 79.753 | 126.077 | 27.744 | 1.00 | 124.15 | 6 |
| ATOM | 4941 | O | SER | C | 179 | 79.622 | 124.932 | 27.308 | 1.00 | 124.27 | 8 |
| ATOM | 4942 | N | GLY | C | 180 | 79.628 | 127.164 | 26.986 | 1.00 | 190.01 | 7 |
| ATOM | 4943 | CA | GLY | C | 180 | 79.316 | 127.065 | 25.570 | 1.00 | 188.94 | 6 |
| ATOM | 4944 | C | GLY | C | 180 | 78.424 | 128.207 | 25.118 | 1.00 | 187.48 | 6 |
| ATOM | 4945 | O | GLY | C | 180 | 78.811 | 129.018 | 24.277 | 1.00 | 187.77 | 8 |
| ATOM | 4946 | N | VAL | C | 181 | 77.222 | 128.255 | 25.684 | 1.00 | 101.50 | 7 |
| ATOM | 4947 | CA | VAL | C | 181 | 76.236 | 129.290 | 25.391 | 1.00 | 98.68 | 6 |
| ATOM | 4948 | CB | VAL | C | 181 | 76.881 | 130.682 | 25.309 | 1.00 | 85.33 | 6 |
| ATOM | 4949 | CG1 | VAL | C | 181 | 75.817 | 131.729 | 25.005 | 1.00 | 86.11 | 6 |
| ATOM | 4950 | CG2 | VAL | C | 181 | 77.590 | 130.999 | 26.616 | 1.00 | 85.64 | 6 |
| ATOM | 4951 | C | VAL | C | 181 | 75.447 | 129.066 | 24.115 | 1.00 | 97.12 | 6 |
| ATOM | 4952 | O | VAL | C | 181 | 75.970 | 129.208 | 23.016 | 1.00 | 98.03 | 8 |
| ATOM | 4953 | N | VAL | C | 182 | 74.174 | 128.728 | 24.283 | 1.00 | 49.17 | 7 |
| ATOM | 4954 | CA | VAL | C | 182 | 73.254 | 128.492 | 23.176 | 1.00 | 47.30 | 6 |
| ATOM | 4955 | CB | VAL | C | 182 | 72.965 | 129.801 | 22.412 | 1.00 | 88.90 | 6 |
| ATOM | 4956 | CG1 | VAL | C | 182 | 71.625 | 129.697 | 21.703 | 1.00 | 88.65 | 6 |
| ATOM | 4957 | CG2 | VAL | C | 182 | 72.958 | 130.978 | 23.380 | 1.00 | 89.47 | 6 |
| ATOM | 4958 | C | VAL | C | 182 | 73.693 | 127.414 | 22.184 | 1.00 | 45.25 | 6 |
| ATOM | 4959 | O | VAL | C | 182 | 74.519 | 127.638 | 21.296 | 1.00 | 44.44 | 8 |
| ATOM | 4960 | N | THR | C | 183 | 73.108 | 126.235 | 22.351 | 1.00 | 54.04 | 7 |
| ATOM | 4961 | CA | THR | C | 183 | 73.398 | 125.098 | 21.499 | 1.00 | 52.78 | 6 |
| ATOM | 4962 | CB | THR | C | 183 | 73.811 | 123.873 | 22.351 | 1.00 | 57.05 | 6 |
| ATOM | 4963 | OG1 | THR | C | 183 | 74.944 | 124.217 | 23.156 | 1.00 | 57.23 | 8 |
| ATOM | 4964 | CG2 | THR | C | 183 | 74.183 | 122.689 | 21.460 | 1.00 | 56.91 | 6 |
| ATOM | 4965 | C | THR | C | 183 | 72.166 | 124.752 | 20.658 | 1.00 | 52.66 | 6 |
| ATOM | 4966 | O | THR | C | 183 | 71.052 | 124.630 | 21.174 | 1.00 | 52.53 | 8 |
| ATOM | 4967 | N | MET | C | 184 | 72.382 | 124.621 | 19.354 | 1.00 | 119.00 | 7 |
| ATOM | 4968 | CA | MET | C | 184 | 71.328 | 124.274 | 18.409 | 1.00 | 118.73 | 6 |
| ATOM | 4969 | CB | MET | C | 184 | 71.469 | 125.124 | 17.139 | 1.00 | 40.44 | 6 |
| ATOM | 4970 | CG | MET | C | 184 | 70.168 | 125.682 | 16.555 | 1.00 | 36.98 | 6 |
| ATOM | 4971 | SD | MET | C | 184 | 69.194 | 124.596 | 15.491 | 1.00 | 33.84 | 16 |
| ATOM | 4972 | CE | MET | C | 184 | 69.676 | 125.144 | 13.911 | 1.00 | 34.18 | 6 |
| ATOM | 4973 | C | MET | C | 184 | 71.586 | 122.805 | 18.088 | 1.00 | 120.26 | 6 |
| ATOM | 4974 | O | MET | C | 184 | 72.524 | 122.489 | 17.353 | 1.00 | 121.69 | 8 |
| ATOM | 4975 | N | LYS | C | 185 | 70.780 | 121.908 | 18.651 | 1.00 | 65.73 | 7 |
| ATOM | 4976 | CA | LYS | C | 185 | 70.981 | 120.488 | 18.397 | 1.00 | 66.80 | 6 |
| ATOM | 4977 | CB | LYS | C | 185 | 71.015 | 119.713 | 19.718 | 1.00 | 112.77 | 6 |
| ATOM | 4978 | CG | LYS | C | 185 | 70.001 | 120.169 | 20.737 | 1.00 | 113.93 | 6 |
| ATOM | 4979 | CD | LYS | C | 185 | 70.298 | 119.579 | 22.108 | 1.00 | 114.95 | 6 |
| ATOM | 4980 | CE | LYS | C | 185 | 69.505 | 120.306 | 23.185 | 1.00 | 115.35 | 6 |
| ATOM | 4981 | NZ | LYS | C | 185 | 69.807 | 119.831 | 24.558 | 1.00 | 116.49 | 7 |
| ATOM | 4982 | C | LYS | C | 185 | 69.997 | 119.834 | 17.435 | 1.00 | 67.41 | 6 |
| ATOM | 4983 | O | LYS | C | 185 | 69.037 | 119.193 | 17.848 | 1.00 | 67.37 | 8 |
| ATOM | 4984 | N | VAL | C | 186 | 70.244 | 119.999 | 16.144 | 1.00 | 63.54 | 7 |
| ATOM | 4985 | CA | VAL | C | 186 | 69.397 | 119.387 | 15.146 | 1.00 | 65.54 | 6 |
| ATOM | 4986 | CB | VAL | C | 186 | 69.662 | 119.980 | 13.767 | 1.00 | 44.28 | 6 |
| ATOM | 4987 | CG1 | VAL | C | 186 | 68.491 | 119.700 | 12.862 | 1.00 | 44.54 | 6 |
| ATOM | 4988 | CG2 | VAL | C | 186 | 69.889 | 121.475 | 13.879 | 1.00 | 43.86 | 6 |
| ATOM | 4989 | C | VAL | C | 186 | 69.828 | 117.928 | 15.183 | 1.00 | 68.57 | 6 |
| ATOM | 4990 | O | VAL | C | 186 | 71.011 | 117.645 | 15.366 | 1.00 | 70.21 | 8 |
| ATOM | 4991 | N | ASN | C | 187 | 68.875 | 117.010 | 15.013 | 1.00 | 73.26 | 7 |
| ATOM | 4992 | CA | ASN | C | 187 | 69.146 | 115.573 | 15.082 | 1.00 | 74.74 | 6 |
| ATOM | 4993 | CB | ASN | C | 187 | 69.404 | 114.964 | 13.684 | 1.00 | 88.07 | 6 |
| ATOM | 4994 | CG | ASN | C | 187 | 70.368 | 115.777 | 12.845 | 1.00 | 89.73 | 6 |
| ATOM | 4995 | OD1 | ASN | C | 187 | 71.370 | 116.274 | 13.345 | 1.00 | 89.09 | 8 |
| ATOM | 4996 | ND2 | ASN | C | 187 | 70.076 | 115.899 | 11.553 | 1.00 | 90.27 | 7 |
| ATOM | 4997 | C | ASN | C | 187 | 70.304 | 115.281 | 16.035 | 1.00 | 75.73 | 6 |
| ATOM | 4998 | O | ASN | C | 187 | 70.241 | 115.632 | 17.212 | 1.00 | 76.73 | 8 |
| ATOM | 4999 | N | LYS | C | 188 | 71.362 | 114.657 | 15.539 | 1.00 | 89.99 | 7 |
| ATOM | 5000 | CA | LYS | C | 188 | 72.506 | 114.332 | 16.388 | 1.00 | 91.65 | 6 |
| ATOM | 5001 | CB | LYS | C | 188 | 72.981 | 112.904 | 16.084 | 1.00 | 132.01 | 6 |
| ATOM | 5002 | CG | LYS | C | 188 | 74.157 | 112.423 | 16.935 | 1.00 | 133.24 | 6 |
| ATOM | 5003 | CD | LYS | C | 188 | 74.602 | 111.012 | 16.538 | 1.00 | 134.93 | 6 |
| ATOM | 5004 | CE | LYS | C | 188 | 75.708 | 110.479 | 17.449 | 1.00 | 136.59 | 6 |
| ATOM | 5005 | NZ | LYS | C | 188 | 76.105 | 109.084 | 17.104 | 1.00 | 137.85 | 7 |
| ATOM | 5006 | C | LYS | C | 188 | 73.668 | 115.309 | 16.212 | 1.00 | 91.77 | 6 |
| ATOM | 5007 | O | LYS | C | 188 | 74.685 | 114.964 | 15.611 | 1.00 | 91.94 | 8 |
| ATOM | 5008 | N | ALA | C | 189 | 73.531 | 116.521 | 16.737 | 1.00 | 100.63 | 7 |
| ATOM | 5009 | CA | ALA | C | 189 | 74.601 | 117.500 | 16.599 | 1.00 | 100.67 | 6 |
| ATOM | 5010 | CB | ALA | C | 189 | 74.513 | 118.170 | 15.240 | 1.00 | 100.93 | 6 |
| ATOM | 5011 | C | ALA | C | 189 | 74.581 | 118.554 | 17.696 | 1.00 | 100.94 | 6 |
| ATOM | 5012 | O | ALA | C | 189 | 73.597 | 118.700 | 18.416 | 1.00 | 101.43 | 8 |
| ATOM | 5013 | N | LYS | C | 190 | 75.682 | 119.285 | 17.817 | 1.00 | 124.83 | 7 |
| ATOM | 5014 | CA | LYS | C | 190 | 75.814 | 120.340 | 18.812 | 1.00 | 123.69 | 6 |
| ATOM | 5015 | CB | LYS | C | 190 | 76.478 | 119.807 | 20.083 | 1.00 | 205.82 | 6 |
| ATOM | 5016 | CG | LYS | C | 190 | 76.921 | 120.904 | 21.045 | 1.00 | 208.87 | 6 |
| ATOM | 5017 | CD | LYS | C | 190 | 77.932 | 120.398 | 22.065 | 1.00 | 208.87 | 6 |
| ATOM | 5018 | CE | LYS | C | 190 | 78.485 | 121.547 | 22.902 | 1.00 | 208.87 | 6 |
| ATOM | 5019 | NZ | LYS | C | 190 | 79.555 | 121.110 | 23.844 | 1.00 | 208.87 | 7 |
| ATOM | 5020 | C | LYS | C | 190 | 76.690 | 121.426 | 18.217 | 1.00 | 122.07 | 6 |
| ATOM | 5021 | O | LYS | C | 190 | 77.913 | 121.319 | 18.244 | 1.00 | 122.53 | 8 |
| ATOM | 5022 | N | PHE | C | 191 | 76.074 | 122.467 | 17.673 | 1.00 | 55.07 | 7 |
| ATOM | 5023 | CA | PHE | C | 191 | 76.843 | 123.553 | 17.083 | 1.00 | 52.94 | 6 |
| ATOM | 5024 | CB | PHE | C | 191 | 76.650 | 123.570 | 15.568 | 1.00 | 54.01 | 6 |
| ATOM | 5025 | CG | PHE | C | 191 | 75.211 | 123.720 | 15.134 | 1.00 | 53.37 | 6 |
| ATOM | 5026 | CD1 | PHE | C | 191 | 74.629 | 124.976 | 15.004 | 1.00 | 53.59 | 6 |
| ATOM | 5027 | CD2 | PHE | C | 191 | 74.444 | 122.606 | 14.836 | 1.00 | 53.02 | 6 |
| ATOM | 5028 | CE1 | PHE | C | 191 | 73.309 | 125.114 | 14.580 | 1.00 | 52.15 | 6 |
| ATOM | 5029 | CE2 | PHE | C | 191 | 73.130 | 122.743 | 14.415 | 1.00 | 52.41 | 6 |
| ATOM | 5030 | CZ | PHE | C | 191 | 72.567 | 123.997 | 14.287 | 1.00 | 51.68 | 6 |
| ATOM | 5031 | C | PHE | C | 191 | 76.433 | 124.890 | 17.663 | 1.00 | 51.32 | 6 |
| ATOM | 5032 | O | PHE | C | 191 | 75.267 | 125.113 | 17.971 | 1.00 | 52.15 | 8 |
| ATOM | 5033 | N | PRO | C | 192 | 77.392 | 125.798 | 17.840 | 1.00 | 38.88 | 7 |
| ATOM | 5034 | CD | PRO | C | 192 | 78.832 | 125.719 | 17.536 | 1.00 | 182.54 | 6 |
| ATOM | 5035 | CA | PRO | C | 192 | 77.037 | 127.108 | 18.392 | 1.00 | 37.55 | 6 |
| ATOM | 5036 | CB | PRO | C | 192 | 78.294 | 127.929 | 18.138 | 1.00 | 182.33 | 6 |
| ATOM | 5037 | CG | PRO | C | 192 | 79.385 | 126.904 | 18.294 | 1.00 | 183.06 | 6 |
| ATOM | 5038 | C | PRO | C | 192 | 75.819 | 127.660 | 17.652 | 1.00 | 35.77 | 6 |
| ATOM | 5039 | O | PRO | C | 192 | 75.675 | 127.450 | 16.446 | 1.00 | 35.21 | 8 |
| ATOM | 5040 | N | LEU | C | 193 | 74.955 | 128.378 | 18.362 | 1.00 | 53.82 | 7 |
| ATOM | 5041 | CA | LEU | C | 193 | 73.741 | 128.907 | 17.743 | 1.00 | 51.81 | 6 |
| ATOM | 5042 | CB | LEU | C | 193 | 72.665 | 129.118 | 18.815 | 1.00 | 143.95 | 6 |
| ATOM | 5043 | CG | LEU | C | 193 | 71.231 | 129.532 | 18.442 | 1.00 | 143.18 | 6 |
| ATOM | 5044 | CD1 | LEU | C | 193 | 71.195 | 131.005 | 18.125 | 1.00 | 143.77 | 6 |
| ATOM | 5045 | CD2 | LEU | C | 193 | 70.715 | 128.716 | 17.275 | 1.00 | 143.01 | 6 |
| ATOM | 5046 | C | LEU | C | 193 | 73.911 | 130.171 | 16.917 | 1.00 | 50.43 | 6 |
| ATOM | 5047 | O | LEU | C | 193 | 73.050 | 130.499 | 16.108 | 1.00 | 50.27 | 8 |
| ATOM | 5048 | N | VAL | C | 194 | 75.012 | 130.882 | 17.108 | 1.00 | 107.98 | 7 |
| ATOM | 5049 | CA | VAL | C | 194 | 75.244 | 132.103 | 16.342 | 1.00 | 107.22 | 6 |
| ATOM | 5050 | CB | VAL | C | 194 | 76.411 | 132.910 | 16.942 | 1.00 | 95.52 | 6 |
| ATOM | 5051 | CG1 | VAL | C | 194 | 76.099 | 134.399 | 16.883 | 1.00 | 94.22 | 6 |
| ATOM | 5052 | CG2 | VAL | C | 194 | 76.679 | 132.452 | 18.373 | 1.00 | 96.45 | 6 |
| ATOM | 5053 | C | VAL | C | 194 | 75.591 | 131.726 | 14.897 | 1.00 | 105.88 | 6 |
| ATOM | 5054 | O | VAL | C | 194 | 75.346 | 132.489 | 13.958 | 1.00 | 104.99 | 8 |
| ATOM | 5055 | N | LEU | C | 195 | 76.150 | 130.527 | 14.743 | 1.00 | 69.97 | 7 |
| ATOM | 5056 | CA | LEU | C | 195 | 76.571 | 129.995 | 13.454 | 1.00 | 67.27 | 6 |
| ATOM | 5057 | CB | LEU | C | 195 | 77.160 | 128.603 | 13.636 | 1.00 | 20.86 | 6 |
| ATOM | 5058 | CG | LEU | C | 195 | 77.876 | 128.058 | 12.409 | 1.00 | 19.45 | 6 |
| ATOM | 5059 | CD1 | LEU | C | 195 | 79.092 | 128.899 | 12.116 | 1.00 | 19.59 | 6 |
| ATOM | 5060 | CD2 | LEU | C | 195 | 78.288 | 126.624 | 12.668 | 1.00 | 19.54 | 6 |
| ATOM | 5061 | C | LEU | C | 195 | 75.461 | 129.928 | 12.427 | 1.00 | 66.47 | 6 |
| ATOM | 5062 | O | LEU | C | 195 | 75.594 | 130.463 | 11.332 | 1.00 | 66.59 | 8 |
| ATOM | 5063 | N | LEU | C | 196 | 74.370 | 129.252 | 12.770 | 1.00 | 54.77 | 7 |
| ATOM | 5064 | CA | LEU | C | 196 | 73.248 | 129.139 | 11.838 | 1.00 | 54.99 | 6 |
| ATOM | 5065 | CB | LEU | C | 196 | 72.178 | 128.167 | 12.353 | 1.00 | 44.73 | 6 |
| ATOM | 5066 | CG | LEU | C | 196 | 70.962 | 128.063 | 11.418 | 1.00 | 44.26 | 6 |
| ATOM | 5067 | CD1 | LEU | C | 196 | 71.049 | 126.752 | 10.631 | 1.00 | 43.98 | 6 |
| ATOM | 5068 | CD2 | LEU | C | 196 | 69.660 | 128.128 | 12.217 | 1.00 | 44.34 | 6 |
| ATOM | 5069 | C | LEU | C | 196 | 72.613 | 130.506 | 11.660 | 1.00 | 55.65 | 6 |
| ATOM | 5070 | O | LEU | C | 196 | 72.135 | 130.849 | 10.571 | 1.00 | 55.48 | 8 |
| ATOM | 5071 | N | LEU | C | 197 | 72.604 | 131.275 | 12.744 | 1.00 | 52.15 | 7 |
| ATOM | 5072 | CA | LEU | C | 197 | 72.028 | 132.600 | 12.711 | 1.00 | 52.47 | 6 |
| ATOM | 5073 | CB | LEU | C | 197 | 72.115 | 133.264 | 14.077 | 1.00 | 16.86 | 6 |
| ATOM | 5074 | CG | LEU | C | 197 | 71.039 | 132.812 | 15.054 | 1.00 | 14.03 | 6 |
| ATOM | 5075 | CD1 | LEU | C | 197 | 71.142 | 133.690 | 16.257 | 1.00 | 14.44 | 6 |
| ATOM | 5076 | CD2 | LEU | C | 197 | 69.645 | 132.920 | 14.450 | 1.00 | 13.87 | 6 |
| ATOM | 5077 | C | LEU | C | 197 | 72.786 | 133.408 | 11.701 | 1.00 | 53.89 | 6 |
| ATOM | 5078 | O | LEU | C | 197 | 72.196 | 134.052 | 10.834 | 1.00 | 54.03 | 8 |
| ATOM | 5079 | N | ARG | C | 198 | 74.106 | 133.357 | 11.809 | 1.00 | 85.09 | 7 |
| ATOM | 5080 | CA | ARG | C | 198 | 74.954 | 134.099 | 10.897 | 1.00 | 87.06 | 6 |
| ATOM | 5081 | CB | ARG | C | 198 | 76.405 | 133.646 | 11.025 | 1.00 | 58.75 | 6 |
| ATOM | 5082 | CG | ARG | C | 198 | 77.378 | 134.771 | 10.830 | 1.00 | 60.92 | 6 |
| ATOM | 5083 | CD | ARG | C | 198 | 76.964 | 135.625 | 9.662 | 1.00 | 63.11 | 6 |
| ATOM | 5084 | NE | ARG | C | 198 | 77.448 | 136.990 | 9.797 | 1.00 | 66.36 | 7 |
| ATOM | 5085 | CZ | ARG | C | 198 | 77.167 | 137.966 | 8.940 | 1.00 | 68.73 | 6 |
| ATOM | 5086 | NH1 | ARG | C | 198 | 76.403 | 137.727 | 7.883 | 1.00 | 69.89 | 7 |
| ATOM | 5087 | NH2 | ARG | C | 198 | 77.649 | 139.185 | 9.143 | 1.00 | 71.74 | 7 |
| ATOM | 5088 | C | ARG | C | 198 | 74.462 | 133.854 | 9.482 | 1.00 | 88.21 | 6 |
| ATOM | 5089 | O | ARG | C | 198 | 74.060 | 134.787 | 8.788 | 1.00 | 88.90 | 8 |
| ATOM | 5090 | N | VAL | C | 199 | 74.491 | 132.591 | 9.067 | 1.00 | 65.98 | 7 |
| ATOM | 5091 | CA | VAL | C | 199 | 74.039 | 132.222 | 7.736 | 1.00 | 66.39 | 6 |
| ATOM | 5092 | CB | VAL | C | 199 | 73.850 | 130.711 | 7.578 | 1.00 | 50.32 | 6 |
| ATOM | 5093 | CG1 | VAL | C | 199 | 73.030 | 130.435 | 6.322 | 1.00 | 50.25 | 6 |
| ATOM | 5094 | CG2 | VAL | C | 199 | 75.209 | 130.015 | 7.489 | 1.00 | 50.24 | 6 |
| ATOM | 5095 | C | VAL | C | 199 | 72.702 | 132.865 | 7.504 | 1.00 | 67.56 | 6 |
| ATOM | 5096 | O | VAL | C | 199 | 72.542 | 133.669 | 6.597 | 1.00 | 69.04 | 8 |
| ATOM | 5097 | N | LEU | C | 200 | 71.738 | 132.503 | 8.338 | 1.00 | 64.55 | 7 |
| ATOM | 5098 | CA | LEU | C | 200 | 70.397 | 133.053 | 8.217 | 1.00 | 66.04 | 6 |
| ATOM | 5099 | CB | LEU | C | 200 | 69.554 | 132.642 | 9.442 | 1.00 | 77.26 | 6 |
| ATOM | 5100 | CG | LEU | C | 200 | 69.452 | 131.141 | 9.778 | 1.00 | 76.80 | 6 |
| ATOM | 5101 | CD1 | LEU | C | 200 | 68.393 | 130.920 | 10.841 | 1.00 | 76.04 | 6 |
| ATOM | 5102 | CD2 | LEU | C | 200 | 69.101 | 130.350 | 8.541 | 1.00 | 76.36 | 6 |
| ATOM | 5103 | C | LEU | C | 200 | 70.447 | 134.588 | 8.055 | 1.00 | 66.96 | 6 |
| ATOM | 5104 | O | LEU | C | 200 | 69.625 | 135.178 | 7.338 | 1.00 | 67.17 | 8 |
| ATOM | 5105 | N | GLY | C | 201 | 71.423 | 135.226 | 8.704 | 1.00 | 58.18 | 7 |
| ATOM | 5106 | CA | GLY | C | 201 | 71.554 | 136.671 | 8.593 | 1.00 | 59.18 | 6 |
| ATOM | 5107 | C | GLY | C | 201 | 71.935 | 137.417 | 9.861 | 1.00 | 58.83 | 6 |
| ATOM | 5108 | O | GLY | C | 201 | 72.865 | 138.220 | 9.857 | 1.00 | 58.22 | 8 |
| ATOM | 5109 | N | TYR | C | 202 | 71.197 | 137.172 | 10.939 | 1.00 | 74.72 | 7 |
| ATOM | 5110 | CA | TYR | C | 202 | 71.468 | 137.827 | 12.212 | 1.00 | 75.90 | 6 |
| ATOM | 5111 | CB | TYR | C | 202 | 70.633 | 137.206 | 13.331 | 1.00 | 67.76 | 6 |
| ATOM | 5112 | CG | TYR | C | 202 | 69.168 | 137.072 | 13.016 | 1.00 | 67.04 | 6 |
| ATOM | 5113 | CD1 | TYR | C | 202 | 68.442 | 135.991 | 13.487 | 1.00 | 66.74 | 6 |
| ATOM | 5114 | CE1 | TYR | C | 202 | 67.096 | 135.862 | 13.214 | 1.00 | 66.84 | 6 |
| ATOM | 5115 | CD2 | TYR | C | 202 | 68.504 | 138.029 | 12.257 | 1.00 | 67.11 | 6 |
| ATOM | 5116 | CE2 | TYR | C | 202 | 67.149 | 137.908 | 11.978 | 1.00 | 66.94 | 6 |
| ATOM | 5117 | CZ | TYR | C | 202 | 66.454 | 136.819 | 12.465 | 1.00 | 66.78 | 6 |
| ATOM | 5118 | OH | TYR | C | 202 | 65.109 | 136.678 | 12.225 | 1.00 | 67.58 | 8 |
| ATOM | 5119 | C | TYR | C | 202 | 72.927 | 137.597 | 12.517 | 1.00 | 77.83 | 6 |
| ATOM | 5120 | O | TYR | C | 202 | 73.511 | 136.618 | 12.059 | 1.00 | 77.94 | 8 |
| ATOM | 5121 | N | ASP | C | 203 | 73.511 | 138.507 | 13.283 | 1.00 | 101.22 | 7 |
| ATOM | 5122 | CA | ASP | C | 203 | 74.908 | 138.415 | 13.673 | 1.00 | 103.17 | 6 |
| ATOM | 5123 | CB | ASP | C | 203 | 75.796 | 138.016 | 12.476 | 1.00 | 128.66 | 6 |
| ATOM | 5124 | CG | ASP | C | 203 | 75.174 | 138.364 | 11.130 | 1.00 | 128.95 | 6 |
| ATOM | 5125 | OD1 | ASP | C | 203 | 74.906 | 139.558 | 10.881 | 1.00 | 131.25 | 8 |
| ATOM | 5126 | OD2 | ASP | C | 203 | 74.948 | 137.432 | 10.324 | 1.00 | 126.78 | 8 |
| ATOM | 5127 | C | ASP | C | 203 | 75.408 | 139.719 | 14.283 | 1.00 | 104.06 | 6 |
| ATOM | 5128 | O | ASP | C | 203 | 75.044 | 140.806 | 13.841 | 1.00 | 103.50 | 8 |
| ATOM | 5129 | N | GLN | C | 204 | 76.235 | 139.580 | 15.314 | 1.00 | 145.69 | 7 |
| ATOM | 5130 | CA | GLN | C | 204 | 76.832 | 140.703 | 16.027 | 1.00 | 146.33 | 6 |
| ATOM | 5131 | CB | GLN | C | 204 | 78.328 | 140.777 | 15.705 | 1.00 | 107.48 | 6 |
| ATOM | 5132 | CG | GLN | C | 204 | 79.041 | 141.959 | 16.342 | 1.00 | 107.70 | 6 |
| ATOM | 5133 | CD | GLN | C | 204 | 78.924 | 141.968 | 17.855 | 1.00 | 108.06 | 6 |
| ATOM | 5134 | OE1 | GLN | C | 204 | 77.823 | 141.894 | 18.405 | 1.00 | 108.82 | 8 |
| ATOM | 5135 | NE2 | GLN | C | 204 | 80.061 | 142.066 | 18.536 | 1.00 | 108.42 | 7 |
| ATOM | 5136 | C | GLN | C | 204 | 76.196 | 142.060 | 15.756 | 1.00 | 146.63 | 6 |
| ATOM | 5137 | O | GLN | C | 204 | 76.219 | 142.549 | 14.628 | 1.00 | 147.43 | 8 |
| ATOM | 5138 | N | GLU | C | 205 | 75.641 | 142.669 | 16.799 | 1.00 | 47.05 | 7 |
| ATOM | 5139 | CA | GLU | C | 205 | 75.018 | 143.986 | 16.687 | 1.00 | 47.71 | 6 |
| ATOM | 5140 | CB | GLU | C | 205 | 75.975 | 144.989 | 16.023 | 1.00 | 151.99 | 6 |
| ATOM | 5141 | CG | GLU | C | 205 | 77.376 | 145.056 | 16.634 | 1.00 | 154.19 | 6 |
| ATOM | 5142 | CD | GLU | C | 205 | 77.376 | 145.395 | 18.116 | 1.00 | 155.63 | 6 |
| ATOM | 5143 | OE1 | GLU | C | 205 | 76.805 | 146.440 | 18.493 | 1.00 | 156.68 | 8 |
| ATOM | 5144 | OE2 | GLU | C | 205 | 77.958 | 144.620 | 18.905 | 1.00 | 155.58 | 8 |
| ATOM | 5145 | C | GLU | C | 205 | 73.708 | 143.954 | 15.910 | 1.00 | 47.75 | 6 |
| ATOM | 5146 | O | GLU | C | 205 | 72.690 | 144.452 | 16.393 | 1.00 | 47.03 | 8 |
| ATOM | 5147 | N | THR | C | 206 | 73.725 | 143.385 | 14.706 | 1.00 | 142.36 | 7 |
| ATOM | 5148 | CA | THR | C | 206 | 72.500 | 143.295 | 13.913 | 1.00 | 143.43 | 6 |
| ATOM | 5149 | CB | THR | C | 206 | 72.783 | 142.845 | 12.447 | 1.00 | 106.22 | 6 |
| ATOM | 5150 | OG1 | THR | C | 206 | 71.713 | 143.282 | 11.600 | 1.00 | 106.37 | 8 |
| ATOM | 5151 | CG2 | THR | C | 206 | 72.885 | 141.331 | 12.346 | 1.00 | 105.31 | 6 |
| ATOM | 5152 | C | THR | C | 206 | 71.636 | 142.255 | 14.621 | 1.00 | 143.64 | 6 |
| ATOM | 5153 | O | THR | C | 206 | 70.408 | 142.250 | 14.505 | 1.00 | 143.65 | 8 |
| ATOM | 5154 | N | LEU | C | 207 | 72.313 | 141.381 | 15.362 | 1.00 | 99.99 | 7 |
| ATOM | 5155 | CA | LEU | C | 207 | 71.677 | 140.325 | 16.139 | 1.00 | 100.36 | 6 |
| ATOM | 5156 | CB | LEU | C | 207 | 72.574 | 139.084 | 16.179 | 1.00 | 70.78 | 6 |
| ATOM | 5157 | CG | LEU | C | 207 | 72.173 | 137.930 | 17.102 | 1.00 | 70.90 | 6 |
| ATOM | 5158 | CD1 | LEU | C | 207 | 70.832 | 137.349 | 16.672 | 1.00 | 70.57 | 6 |
| ATOM | 5159 | CD2 | LEU | C | 207 | 73.257 | 136.865 | 17.058 | 1.00 | 71.36 | 6 |
| ATOM | 5160 | C | LEU | C | 207 | 71.510 | 140.866 | 17.548 | 1.00 | 100.14 | 6 |
| ATOM | 5161 | O | LEU | C | 207 | 70.521 | 141.525 | 17.868 | 1.00 | 100.30 | 8 |
| ATOM | 5162 | N | VAL | C | 208 | 72.499 | 140.584 | 18.384 | 1.00 | 68.98 | 7 |
| ATOM | 5163 | CA | VAL | C | 208 | 72.482 | 141.050 | 19.756 | 1.00 | 69.54 | 6 |
| ATOM | 5164 | CB | VAL | C | 208 | 73.693 | 140.514 | 20.533 | 1.00 | 156.59 | 6 |
| ATOM | 5165 | CG1 | VAL | C | 208 | 73.675 | 141.048 | 21.954 | 1.00 | 157.26 | 6 |
| ATOM | 5166 | CG2 | VAL | C | 208 | 73.673 | 138.994 | 20.528 | 1.00 | 157.20 | 6 |
| ATOM | 5167 | C | VAL | C | 208 | 72.516 | 142.574 | 19.767 | 1.00 | 69.61 | 6 |
| ATOM | 5168 | O | VAL | C | 208 | 72.927 | 143.204 | 18.789 | 1.00 | 69.37 | 8 |
| ATOM | 5169 | N | ARG | C | 209 | 72.086 | 143.161 | 20.879 | 1.00 | 102.05 | 7 |
| ATOM | 5170 | CA | ARG | C | 209 | 72.062 | 144.607 | 21.000 | 1.00 | 102.46 | 6 |
| ATOM | 5171 | CB | ARG | C | 209 | 73.443 | 145.164 | 20.650 | 1.00 | 80.09 | 6 |
| ATOM | 5172 | CG | ARG | C | 209 | 74.536 | 144.483 | 21.457 | 1.00 | 80.04 | 6 |
| ATOM | 5173 | CD | ARG | C | 209 | 75.935 | 145.015 | 21.198 | 1.00 | 80.98 | 6 |
| ATOM | 5174 | NE | ARG | C | 209 | 76.830 | 144.580 | 22.271 | 1.00 | 82.65 | 7 |
| ATOM | 5175 | CZ | ARG | C | 209 | 78.128 | 144.861 | 22.351 | 1.00 | 83.21 | 6 |
| ATOM | 5176 | NH1 | ARG | C | 209 | 78.720 | 145.588 | 21.408 | 1.00 | 83.30 | 7 |
| ATOM | 5177 | NH2 | ARG | C | 209 | 78.831 | 144.424 | 23.391 | 1.00 | 82.93 | 7 |
| ATOM | 5178 | C | ARG | C | 209 | 70.998 | 145.061 | 20.014 | 1.00 | 103.27 | 6 |
| ATOM | 5179 | O | ARG | C | 209 | 70.996 | 146.191 | 19.535 | 1.00 | 103.27 | 8 |
| ATOM | 5180 | N | GLU | C | 210 | 70.089 | 144.136 | 19.727 | 1.00 | 118.85 | 7 |
| ATOM | 5181 | CA | GLU | C | 210 | 68.984 | 144.358 | 18.810 | 1.00 | 120.27 | 6 |
| ATOM | 5182 | CB | GLU | C | 210 | 69.503 | 144.441 | 17.372 | 1.00 | 180.96 | 6 |
| ATOM | 5183 | CG | GLU | C | 210 | 68.659 | 145.317 | 16.459 | 1.00 | 181.78 | 6 |
| ATOM | 5184 | CD | GLU | C | 210 | 68.723 | 146.784 | 16.840 | 1.00 | 182.17 | 6 |
| ATOM | 5185 | OE1 | GLU | C | 210 | 69.801 | 147.394 | 16.677 | 1.00 | 182.81 | 8 |
| ATOM | 5186 | OE2 | GLU | C | 210 | 67.699 | 147.325 | 17.308 | 1.00 | 182.12 | 8 |
| ATOM | 5187 | C | GLU | C | 210 | 68.034 | 143.163 | 18.970 | 1.00 | 120.32 | 6 |
| ATOM | 5188 | O | GLU | C | 210 | 66.988 | 143.079 | 18.319 | 1.00 | 120.10 | 8 |
| ATOM | 5189 | N | LEU | C | 211 | 68.427 | 142.237 | 19.843 | 1.00 | 116.71 | 7 |
| ATOM | 5190 | CA | LEU | C | 211 | 67.643 | 141.041 | 20.155 | 1.00 | 115.98 | 6 |
| ATOM | 5191 | CB | LEU | C | 211 | 68.133 | 139.830 | 19.357 | 1.00 | 135.44 | 6 |
| ATOM | 5192 | CG | LEU | C | 211 | 67.869 | 138.488 | 20.064 | 1.00 | 136.01 | 6 |
| ATOM | 5193 | CD1 | LEU | C | 211 | 66.374 | 138.278 | 20.222 | 1.00 | 137.05 | 6 |
| ATOM | 5194 | CD2 | LEU | C | 211 | 68.488 | 137.344 | 19.284 | 1.00 | 136.63 | 6 |
| ATOM | 5195 | C | LEU | C | 211 | 67.794 | 140.719 | 21.633 | 1.00 | 114.77 | 6 |
| ATOM | 5196 | O | LEU | C | 211 | 66.827 | 140.715 | 22.398 | 1.00 | 114.10 | 8 |
| ATOM | 5197 | N | SER | C | 212 | 69.033 | 140.437 | 22.010 | 1.00 | 109.57 | 7 |
| ATOM | 5198 | CA | SER | C | 212 | 69.373 | 140.091 | 23.375 | 1.00 | 109.17 | 6 |
| ATOM | 5199 | CB | SER | C | 212 | 70.315 | 138.897 | 23.368 | 1.00 | 83.30 | 6 |
| ATOM | 5200 | OG | SER | C | 212 | 71.413 | 139.150 | 22.509 | 1.00 | 83.23 | 8 |
| ATOM | 5201 | C | SER | C | 212 | 70.036 | 141.259 | 24.084 | 1.00 | 109.05 | 6 |
| ATOM | 5202 | O | SER | C | 212 | 71.153 | 141.650 | 23.747 | 1.00 | 109.05 | 8 |
| ATOM | 5203 | N | ALA | C | 213 | 69.342 | 141.814 | 25.070 | 1.00 | 89.80 | 7 |
| ATOM | 5204 | CA | ALA | C | 213 | 69.866 | 142.935 | 25.834 | 1.00 | 88.67 | 6 |
| ATOM | 5205 | CB | ALA | C | 213 | 69.444 | 144.247 | 25.191 | 1.00 | 119.25 | 6 |
| ATOM | 5206 | C | ALA | C | 213 | 69.365 | 142.870 | 27.268 | 1.00 | 88.05 | 6 |
| ATOM | 5207 | O | ALA | C | 213 | 70.162 | 142.791 | 28.203 | 1.00 | 87.84 | 8 |
| ATOM | 5208 | N | TYR | C | 214 | 68.046 | 142.888 | 27.438 | 1.00 | 129.04 | 7 |
| ATOM | 5209 | CA | TYR | C | 214 | 67.468 | 142.842 | 28.771 | 1.00 | 128.86 | 6 |
| ATOM | 5210 | CB | TYR | C | 214 | 65.933 | 142.921 | 28.712 | 1.00 | 81.89 | 6 |
| ATOM | 5211 | CG | TYR | C | 214 | 65.215 | 141.897 | 27.840 | 1.00 | 80.30 | 6 |
| ATOM | 5212 | CD1 | TYR | C | 214 | 65.777 | 140.642 | 27.553 | 1.00 | 79.07 | 6 |
| ATOM | 5213 | CE1 | TYR | C | 214 | 65.081 | 139.705 | 26.781 | 1.00 | 77.16 | 6 |
| ATOM | 5214 | CD2 | TYR | C | 214 | 63.939 | 142.175 | 27.332 | 1.00 | 79.27 | 6 |
| ATOM | 5215 | CE2 | TYR | C | 214 | 63.242 | 141.247 | 26.569 | 1.00 | 77.29 | 6 |
| ATOM | 5216 | CZ | TYR | C | 214 | 63.815 | 140.022 | 26.294 | 1.00 | 76.39 | 6 |
| ATOM | 5217 | OH | TYR | C | 214 | 63.124 | 139.140 | 25.501 | 1.00 | 75.89 | 8 |
| ATOM | 5218 | C | TYR | C | 214 | 67.901 | 141.630 | 29.592 | 1.00 | 128.94 | 6 |
| ATOM | 5219 | O | TYR | C | 214 | 67.635 | 141.554 | 30.793 | 1.00 | 129.75 | 8 |
| ATOM | 5220 | N | GLY | C | 215 | 68.573 | 140.686 | 28.948 | 1.00 | 75.08 | 7 |
| ATOM | 5221 | CA | GLY | C | 215 | 69.038 | 139.515 | 29.660 | 1.00 | 75.27 | 6 |
| ATOM | 5222 | C | GLY | C | 215 | 70.350 | 139.026 | 29.079 | 1.00 | 75.58 | 6 |
| ATOM | 5223 | O | GLY | C | 215 | 70.350 | 138.096 | 28.268 | 1.00 | 76.28 | 8 |
| ATOM | 5224 | N | ASP | C | 216 | 71.458 | 139.648 | 29.489 | 1.00 | 85.58 | 7 |
| ATOM | 5225 | CA | ASP | C | 216 | 72.804 | 139.300 | 29.007 | 1.00 | 85.27 | 6 |
| ATOM | 5226 | CB | ASP | C | 216 | 73.791 | 139.261 | 30.174 | 1.00 | 131.92 | 6 |
| ATOM | 5227 | CG | ASP | C | 216 | 73.854 | 140.569 | 30.922 | 1.00 | 132.48 | 6 |
| ATOM | 5228 | OD1 | ASP | C | 216 | 72.816 | 140.969 | 31.485 | 1.00 | 132.94 | 8 |
| ATOM | 5229 | OD2 | ASP | C | 216 | 74.937 | 141.197 | 30.945 | 1.00 | 132.30 | 8 |
| ATOM | 5230 | C | ASP | C | 216 | 72.876 | 137.972 | 28.266 | 1.00 | 84.97 | 6 |
| ATOM | 5231 | O | ASP | C | 216 | 72.381 | 137.829 | 27.146 | 1.00 | 84.91 | 8 |
| ATOM | 5232 | N | LEU | C | 217 | 73.514 | 136.998 | 28.894 | 1.00 | 77.58 | 7 |
| ATOM | 5233 | CA | LEU | C | 217 | 73.623 | 135.687 | 28.289 | 1.00 | 77.89 | 6 |
| ATOM | 5234 | CB | LEU | C | 217 | 72.237 | 135.044 | 28.190 | 1.00 | 80.27 | 6 |
| ATOM | 5235 | CG | LEU | C | 217 | 71.155 | 135.447 | 29.201 | 1.00 | 79.36 | 6 |
| ATOM | 5236 | CD1 | LEU | C | 217 | 69.855 | 134.737 | 28.857 | 1.00 | 78.34 | 6 |
| ATOM | 5237 | CD2 | LEU | C | 217 | 71.590 | 135.106 | 30.617 | 1.00 | 78.99 | 6 |
| ATOM | 5238 | C | LEU | C | 217 | 74.189 | 135.836 | 26.887 | 1.00 | 78.60 | 6 |
| ATOM | 5239 | O | LEU | C | 217 | 74.174 | 134.882 | 26.111 | 1.00 | 79.02 | 8 |
| ATOM | 5240 | N | VAL | C | 218 | 74.698 | 137.022 | 26.562 | 1.00 | 130.42 | 7 |
| ATOM | 5241 | CA | VAL | C | 218 | 75.226 | 137.263 | 25.225 | 1.00 | 131.09 | 6 |
| ATOM | 5242 | CB | VAL | C | 218 | 74.759 | 138.612 | 24.678 | 1.00 | 90.38 | 6 |
| ATOM | 5243 | CG1 | VAL | C | 218 | 75.311 | 138.810 | 23.279 | 1.00 | 90.04 | 6 |
| ATOM | 5244 | CG2 | VAL | C | 218 | 73.247 | 138.664 | 24.660 | 1.00 | 89.65 | 6 |
| ATOM | 5245 | C | VAL | C | 218 | 76.730 | 137.185 | 25.009 | 1.00 | 131.97 | 6 |
| ATOM | 5246 | O | VAL | C | 218 | 77.164 | 136.633 | 24.000 | 1.00 | 132.21 | 8 |
| ATOM | 5247 | N | GLN | C | 219 | 77.527 | 137.744 | 25.916 | 1.00 | 152.90 | 7 |
| ATOM | 5248 | CA | GLN | C | 219 | 78.980 | 137.695 | 25.747 | 1.00 | 153.25 | 6 |
| ATOM | 5249 | CB | GLN | C | 219 | 79.700 | 137.907 | 27.080 | 1.00 | 136.00 | 6 |
| ATOM | 5250 | CG | GLN | C | 219 | 79.550 | 139.285 | 27.687 | 1.00 | 135.38 | 6 |
| ATOM | 5251 | CD | GLN | C | 219 | 80.500 | 139.495 | 28.856 | 1.00 | 136.08 | 6 |
| ATOM | 5252 | OE1 | GLN | C | 219 | 81.719 | 139.563 | 28.675 | 1.00 | 136.00 | 8 |
| ATOM | 5253 | NE2 | GLN | C | 219 | 79.948 | 139.586 | 30.063 | 1.00 | 136.33 | 7 |
| ATOM | 5254 | C | GLN | C | 219 | 79.402 | 136.345 | 25.168 | 1.00 | 153.29 | 6 |
| ATOM | 5255 | O | GLN | C | 219 | 80.380 | 136.250 | 24.424 | 1.00 | 153.73 | 8 |
| ATOM | 5256 | N | GLY | C | 220 | 78.649 | 135.305 | 25.516 | 1.00 | 75.93 | 7 |
| ATOM | 5257 | CA | GLY | C | 220 | 78.941 | 133.974 | 25.027 | 1.00 | 77.21 | 6 |
| ATOM | 5258 | C | GLY | C | 220 | 78.353 | 133.704 | 23.653 | 1.00 | 78.18 | 6 |
| ATOM | 5259 | O | GLY | C | 220 | 78.902 | 132.907 | 22.886 | 1.00 | 78.07 | 8 |
| ATOM | 5260 | N | LEU | C | 221 | 77.229 | 134.342 | 23.338 | 1.00 | 83.09 | 7 |
| ATOM | 5261 | CA | LEU | C | 221 | 76.611 | 134.157 | 22.029 | 1.00 | 84.37 | 6 |
| ATOM | 5262 | CB | LEU | C | 221 | 75.195 | 134.734 | 22.011 | 1.00 | 57.92 | 6 |
| ATOM | 5263 | CG | LEU | C | 221 | 74.439 | 134.511 | 20.695 | 1.00 | 57.02 | 6 |
| ATOM | 5264 | CD1 | LEU | C | 221 | 73.967 | 133.054 | 20.628 | 1.00 | 56.56 | 6 |
| ATOM | 5265 | CD2 | LEU | C | 221 | 73.259 | 135.474 | 20.585 | 1.00 | 56.14 | 6 |
| ATOM | 5266 | C | LEU | C | 221 | 77.476 | 134.892 | 21.003 | 1.00 | 85.81 | 6 |
| ATOM | 5267 | O | LEU | C | 221 | 77.161 | 134.943 | 19.812 | 1.00 | 86.07 | 8 |
| ATOM | 5268 | N | LEU | C | 222 | 78.578 | 135.460 | 21.479 | 1.00 | 56.83 | 7 |
| ATOM | 5269 | CA | LEU | C | 222 | 79.482 | 136.193 | 20.615 | 1.00 | 58.88 | 6 |
| ATOM | 5270 | CB | LEU | C | 222 | 79.536 | 137.662 | 21.056 | 1.00 | 71.27 | 6 |
| ATOM | 5271 | CG | LEU | C | 222 | 78.229 | 138.468 | 20.951 | 1.00 | 70.06 | 6 |
| ATOM | 5272 | CD1 | LEU | C | 222 | 78.407 | 139.815 | 21.614 | 1.00 | 69.38 | 6 |
| ATOM | 5273 | CD2 | LEU | C | 222 | 77.833 | 138.649 | 19.497 | 1.00 | 68.39 | 6 |
| ATOM | 5274 | C | LEU | C | 222 | 80.888 | 135.593 | 20.583 | 1.00 | 61.05 | 6 |
| ATOM | 5275 | O | LEU | C | 222 | 81.616 | 135.797 | 19.620 | 1.00 | 62.44 | 8 |
| ATOM | 5276 | N | ASP | C | 223 | 81.270 | 134.848 | 21.617 | 1.00 | 149.38 | 7 |
| ATOM | 5277 | CA | ASP | C | 223 | 82.604 | 134.236 | 21.662 | 1.00 | 151.90 | 6 |
| ATOM | 5278 | CB | ASP | C | 223 | 82.637 | 133.082 | 22.671 | 1.00 | 208.87 | 6 |
| ATOM | 5279 | CG | ASP | C | 223 | 82.824 | 133.561 | 24.099 | 1.00 | 208.87 | 6 |
| ATOM | 5280 | OD1 | ASP | C | 223 | 83.824 | 134.261 | 24.364 | 1.00 | 208.87 | 8 |
| ATOM | 5281 | OD2 | ASP | C | 223 | 81.975 | 133.236 | 24.956 | 1.00 | 208.87 | 8 |
| ATOM | 5282 | C | ASP | C | 223 | 83.092 | 133.731 | 20.303 | 1.00 | 152.75 | 6 |
| ATOM | 5283 | O | ASP | C | 223 | 84.259 | 133.916 | 19.950 | 1.00 | 152.57 | 8 |
| ATOM | 5284 | N | GLU | C | 224 | 82.202 | 133.089 | 19.549 | 1.00 | 84.62 | 7 |
| ATOM | 5285 | CA | GLU | C | 224 | 82.549 | 132.576 | 18.228 | 1.00 | 86.27 | 6 |
| ATOM | 5286 | CB | GLU | C | 224 | 81.940 | 131.186 | 18.019 | 1.00 | 208.87 | 6 |
| ATOM | 5287 | CG | GLU | C | 224 | 82.704 | 130.066 | 18.706 | 1.00 | 208.87 | 6 |
| ATOM | 5288 | CD | GLU | C | 224 | 84.113 | 129.907 | 18.162 | 1.00 | 208.87 | 6 |
| ATOM | 5289 | OE1 | GLU | C | 224 | 84.265 | 129.623 | 16.955 | 1.00 | 208.87 | 8 |
| ATOM | 5290 | OE2 | GLU | C | 224 | 85.073 | 130.069 | 18.943 | 1.00 | 208.87 | 8 |
| ATOM | 5291 | C | GLU | C | 224 | 82.085 | 133.520 | 17.117 | 1.00 | 86.52 | 6 |
| ATOM | 5292 | O | GLU | C | 224 | 81.729 | 133.075 | 16.020 | 1.00 | 85.93 | 8 |
| ATOM | 5293 | N | ALA | C | 225 | 82.091 | 134.822 | 17.406 | 1.00 | 175.86 | 7 |
| ATOM | 5294 | CA | ALA | C | 225 | 81.689 | 135.831 | 16.426 | 1.00 | 176.34 | 6 |
| ATOM | 5295 | CB | ALA | C | 225 | 81.941 | 137.230 | 16.961 | 1.00 | 78.67 | 6 |
| ATOM | 5296 | C | ALA | C | 225 | 82.538 | 135.586 | 15.199 | 1.00 | 176.29 | 6 |
| ATOM | 5297 | O | ALA | C | 225 | 82.205 | 136.013 | 14.095 | 1.00 | 176.67 | 8 |
| ATOM | 5298 | N | VAL | C | 226 | 83.657 | 134.904 | 15.419 | 1.00 | 98.39 | 7 |
| ATOM | 5299 | CA | VAL | C | 226 | 84.548 | 134.549 | 14.341 | 1.00 | 97.87 | 6 |
| ATOM | 5300 | CB | VAL | C | 226 | 85.628 | 133.561 | 14.831 | 1.00 | 88.32 | 6 |
| ATOM | 5301 | CG1 | VAL | C | 226 | 86.463 | 134.211 | 15.916 | 1.00 | 88.30 | 6 |
| ATOM | 5302 | CG2 | VAL | C | 226 | 84.985 | 132.302 | 15.377 | 1.00 | 88.42 | 6 |
| ATOM | 5303 | C | VAL | C | 226 | 83.635 | 133.881 | 13.319 | 1.00 | 97.91 | 6 |
| ATOM | 5304 | O | VAL | C | 226 | 83.751 | 134.119 | 12.119 | 1.00 | 98.63 | 8 |
| ATOM | 5305 | N | LEU | C | 227 | 82.709 | 133.060 | 13.814 | 1.00 | 138.81 | 7 |
| ATOM | 5306 | CA | LEU | C | 227 | 81.743 | 132.375 | 12.964 | 1.00 | 139.05 | 6 |
| ATOM | 5307 | CB | LEU | C | 227 | 80.978 | 131.303 | 13.755 | 1.00 | 116.19 | 6 |
| ATOM | 5308 | CG | LEU | C | 227 | 81.749 | 130.059 | 14.213 | 1.00 | 117.20 | 6 |
| ATOM | 5309 | CD1 | LEU | C | 227 | 80.814 | 129.071 | 14.911 | 1.00 | 116.00 | 6 |
| ATOM | 5310 | CD2 | LEU | C | 227 | 82.392 | 129.404 | 13.005 | 1.00 | 117.61 | 6 |
| ATOM | 5311 | C | LEU | C | 227 | 80.763 | 133.415 | 12.437 | 1.00 | 138.64 | 6 |
| ATOM | 5312 | O | LEU | C | 227 | 80.368 | 133.379 | 11.275 | 1.00 | 138.91 | 8 |
| ATOM | 5313 | N | ALA | C | 228 | 80.384 | 134.346 | 13.306 | 1.00 | 65.37 | 7 |
| ATOM | 5314 | CA | ALA | C | 228 | 79.458 | 135.424 | 12.952 | 1.00 | 65.33 | 6 |
| ATOM | 5315 | CB | ALA | C | 228 | 78.777 | 135.959 | 14.211 | 1.00 | 126.31 | 6 |
| ATOM | 5316 | C | ALA | C | 228 | 80.143 | 136.578 | 12.213 | 1.00 | 65.44 | 6 |
| ATOM | 5317 | O | ALA | C | 228 | 79.798 | 137.742 | 12.416 | 1.00 | 65.01 | 8 |
| ATOM | 5318 | N | MET | C | 229 | 81.103 | 136.245 | 11.353 | 1.00 | 105.03 | 7 |
| ATOM | 5319 | CA | MET | C | 229 | 81.842 | 137.245 | 10.588 | 1.00 | 104.42 | 6 |
| ATOM | 5320 | CB | MET | C | 229 | 83.326 | 137.179 | 10.950 | 1.00 | 208.87 | 6 |
| ATOM | 5321 | CG | MET | C | 229 | 83.622 | 137.635 | 12.361 | 1.00 | 208.87 | 6 |
| ATOM | 5322 | SD | MET | C | 229 | 82.935 | 139.271 | 12.666 | 1.00 | 208.87 | 16 |
| ATOM | 5323 | CE | MET | C | 229 | 84.185 | 140.325 | 11.847 | 1.00 | 208.87 | 6 |
| ATOM | 5324 | C | MET | C | 229 | 81.686 | 137.148 | 9.068 | 1.00 | 102.40 | 6 |
| ATOM | 5325 | O | MET | C | 229 | 82.146 | 138.029 | 8.341 | 1.00 | 101.26 | 8 |
| ATOM | 5326 | N | ARG | C | 230 | 81.046 | 136.085 | 8.590 | 1.00 | 66.08 | 7 |
| ATOM | 5327 | CA | ARG | C | 230 | 80.836 | 135.900 | 7.157 | 1.00 | 65.23 | 6 |
| ATOM | 5328 | CB | ARG | C | 230 | 82.188 | 135.731 | 6.454 | 1.00 | 105.16 | 6 |
| ATOM | 5329 | CG | ARG | C | 230 | 83.196 | 134.900 | 7.239 | 1.00 | 106.81 | 6 |
| ATOM | 5330 | CD | ARG | C | 230 | 84.492 | 134.702 | 6.457 | 1.00 | 107.45 | 6 |
| ATOM | 5331 | NE | ARG | C | 230 | 85.550 | 134.118 | 7.281 | 1.00 | 108.11 | 7 |
| ATOM | 5332 | CZ | ARG | C | 230 | 86.117 | 134.732 | 8.316 | 1.00 | 108.21 | 6 |
| ATOM | 5333 | NH1 | ARG | C | 230 | 85.732 | 135.953 | 8.659 | 1.00 | 107.49 | 7 |
| ATOM | 5334 | NH2 | ARG | C | 230 | 87.069 | 134.123 | 9.010 | 1.00 | 109.17 | 7 |
| ATOM | 5335 | C | ARG | C | 230 | 79.937 | 134.708 | 6.824 | 1.00 | 63.26 | 6 |
| ATOM | 5336 | O | ARG | C | 230 | 80.314 | 133.558 | 7.054 | 1.00 | 63.36 | 8 |
| ATOM | 5337 | N | PRO | C | 231 | 78.733 | 134.962 | 6.275 | 1.00 | 140.24 | 7 |
| ATOM | 5338 | CD | PRO | C | 231 | 78.185 | 136.252 | 5.826 | 1.00 | 42.06 | 6 |
| ATOM | 5339 | CA | PRO | C | 231 | 77.822 | 133.864 | 5.926 | 1.00 | 138.58 | 6 |
| ATOM | 5340 | CB | PRO | C | 231 | 76.584 | 134.590 | 5.399 | 1.00 | 40.00 | 6 |
| ATOM | 5341 | CG | PRO | C | 231 | 77.154 | 135.818 | 4.804 | 1.00 | 41.60 | 6 |
| ATOM | 5342 | C | PRO | C | 231 | 78.474 | 132.999 | 4.866 | 1.00 | 137.86 | 6 |
| ATOM | 5343 | O | PRO | C | 231 | 79.656 | 133.162 | 4.589 | 1.00 | 139.31 | 8 |
| ATOM | 5344 | N | GLU | C | 232 | 77.715 | 132.091 | 4.267 | 1.00 | 77.90 | 7 |
| ATOM | 5345 | CA | GLU | C | 232 | 78.268 | 131.224 | 3.231 | 1.00 | 75.34 | 6 |
| ATOM | 5346 | CB | GLU | C | 232 | 78.744 | 132.063 | 2.036 | 1.00 | 99.87 | 6 |
| ATOM | 5347 | CG | GLU | C | 232 | 79.165 | 131.268 | 0.789 | 1.00 | 100.05 | 6 |
| ATOM | 5348 | CD | GLU | C | 232 | 80.578 | 130.706 | 0.863 | 1.00 | 99.76 | 6 |
| ATOM | 5349 | OE1 | GLU | C | 232 | 80.817 | 129.769 | 1.652 | 1.00 | 99.37 | 8 |
| ATOM | 5350 | OE2 | GLU | C | 232 | 81.451 | 131.208 | 0.122 | 1.00 | 100.08 | 8 |
| ATOM | 5351 | C | GLU | C | 232 | 79.435 | 130.438 | 3.810 | 1.00 | 73.40 | 6 |
| ATOM | 5352 | O | GLU | C | 232 | 79.314 | 129.245 | 4.083 | 1.00 | 73.09 | 8 |
| ATOM | 5353 | N | GLU | C | 233 | 80.569 | 131.110 | 3.985 | 1.00 | 73.71 | 7 |
| ATOM | 5354 | CA | GLU | C | 233 | 81.753 | 130.488 | 4.561 | 1.00 | 71.34 | 6 |
| ATOM | 5355 | CB | GLU | C | 233 | 82.771 | 131.564 | 4.953 | 1.00 | 69.36 | 6 |
| ATOM | 5356 | CG | GLU | C | 233 | 84.231 | 131.127 | 4.910 | 1.00 | 67.28 | 6 |
| ATOM | 5357 | CD | GLU | C | 233 | 84.578 | 130.039 | 5.918 | 1.00 | 66.29 | 6 |
| ATOM | 5358 | OE1 | GLU | C | 233 | 84.483 | 130.293 | 7.137 | 1.00 | 65.59 | 8 |
| ATOM | 5359 | OE2 | GLU | C | 233 | 84.957 | 128.927 | 5.493 | 1.00 | 66.13 | 8 |
| ATOM | 5360 | C | GLU | C | 233 | 81.224 | 129.791 | 5.803 | 1.00 | 70.23 | 6 |
| ATOM | 5361 | O | GLU | C | 233 | 81.790 | 128.802 | 6.274 | 1.00 | 70.93 | 8 |
| ATOM | 5362 | N | ALA | C | 234 | 80.107 | 130.323 | 6.302 | 1.00 | 99.90 | 7 |
| ATOM | 5363 | CA | ALA | C | 234 | 79.418 | 129.814 | 7.483 | 1.00 | 97.62 | 6 |
| ATOM | 5364 | CB | ALA | C | 234 | 78.408 | 130.846 | 7.971 | 1.00 | 76.06 | 6 |
| ATOM | 5365 | C | ALA | C | 234 | 78.719 | 128.473 | 7.229 | 1.00 | 95.90 | 6 |
| ATOM | 5366 | O | ALA | C | 234 | 79.022 | 127.481 | 7.899 | 1.00 | 95.75 | 8 |
| ATOM | 5367 | N | MET | C | 235 | 77.783 | 128.442 | 6.278 | 1.00 | 65.06 | 7 |
| ATOM | 5368 | CA | MET | C | 235 | 77.074 | 127.203 | 5.954 | 1.00 | 62.59 | 6 |
| ATOM | 5369 | CB | MET | C | 235 | 76.397 | 127.298 | 4.594 | 1.00 | 48.98 | 6 |
| ATOM | 5370 | CG | MET | C | 235 | 75.708 | 128.597 | 4.319 | 1.00 | 48.48 | 6 |
| ATOM | 5371 | SD | MET | C | 235 | 74.890 | 128.508 | 2.715 | 1.00 | 48.43 | 16 |
| ATOM | 5372 | CE | MET | C | 235 | 73.125 | 128.459 | 3.250 | 1.00 | 47.57 | 6 |
| ATOM | 5373 | C | MET | C | 235 | 78.097 | 126.075 | 5.879 | 1.00 | 62.17 | 6 |
| ATOM | 5374 | O | MET | C | 235 | 77.866 | 124.969 | 6.374 | 1.00 | 61.98 | 8 |
| ATOM | 5375 | N | VAL | C | 236 | 79.230 | 126.374 | 5.247 | 1.00 | 38.65 | 7 |
| ATOM | 5376 | CA | VAL | C | 236 | 80.299 | 125.405 | 5.082 | 1.00 | 36.92 | 6 |
| ATOM | 5377 | CB | VAL | C | 236 | 81.394 | 125.946 | 4.140 | 1.00 | 13.87 | 6 |
| ATOM | 5378 | CG1 | VAL | C | 236 | 82.629 | 125.036 | 4.205 | 1.00 | 13.87 | 6 |
| ATOM | 5379 | CG2 | VAL | C | 236 | 80.836 | 126.039 | 2.695 | 1.00 | 13.87 | 6 |
| ATOM | 5380 | C | VAL | C | 236 | 80.909 | 124.982 | 6.409 | 1.00 | 37.57 | 6 |
| ATOM | 5381 | O | VAL | C | 236 | 81.212 | 123.811 | 6.596 | 1.00 | 37.03 | 8 |
| ATOM | 5382 | N | ARG | C | 237 | 81.097 | 125.917 | 7.331 | 1.00 | 101.36 | 7 |
| ATOM | 5383 | CA | ARG | C | 237 | 81.646 | 125.545 | 8.625 | 1.00 | 103.24 | 6 |
| ATOM | 5384 | CB | ARG | C | 237 | 81.927 | 126.786 | 9.464 | 1.00 | 117.69 | 6 |
| ATOM | 5385 | CG | ARG | C | 237 | 82.957 | 127.707 | 8.842 | 1.00 | 119.98 | 6 |
| ATOM | 5386 | CD | ARG | C | 237 | 84.320 | 127.050 | 8.754 | 1.00 | 121.93 | 6 |
| ATOM | 5387 | NE | ARG | C | 237 | 85.291 | 127.934 | 8.115 | 1.00 | 125.29 | 7 |
| ATOM | 5388 | CZ | ARG | C | 237 | 86.586 | 127.662 | 7.993 | 1.00 | 126.39 | 6 |
| ATOM | 5389 | NH1 | ARG | C | 237 | 87.075 | 126.526 | 8.471 | 1.00 | 127.88 | 7 |
| ATOM | 5390 | NH2 | ARG | C | 237 | 87.393 | 128.525 | 7.390 | 1.00 | 126.26 | 7 |
| ATOM | 5391 | C | ARG | C | 237 | 80.608 | 124.657 | 9.305 | 1.00 | 103.63 | 6 |
| ATOM | 5392 | O | ARG | C | 237 | 80.951 | 123.662 | 9.943 | 1.00 | 104.18 | 8 |
| ATOM | 5393 | N | LEU | C | 238 | 79.337 | 125.022 | 9.157 | 1.00 | 56.15 | 7 |
| ATOM | 5394 | CA | LEU | C | 238 | 78.240 | 124.239 | 9.723 | 1.00 | 56.26 | 6 |
| ATOM | 5395 | CB | LEU | C | 238 | 76.903 | 124.969 | 9.511 | 1.00 | 20.59 | 6 |
| ATOM | 5396 | CG | LEU | C | 238 | 75.548 | 124.426 | 9.996 | 1.00 | 18.61 | 6 |
| ATOM | 5397 | CD1 | LEU | C | 238 | 75.416 | 124.515 | 11.505 | 1.00 | 17.53 | 6 |
| ATOM | 5398 | CD2 | LEU | C | 238 | 74.454 | 125.252 | 9.356 | 1.00 | 17.41 | 6 |
| ATOM | 5399 | C | LEU | C | 238 | 78.239 | 122.899 | 8.977 | 1.00 | 57.78 | 6 |
| ATOM | 5400 | O | LEU | C | 238 | 78.290 | 121.838 | 9.594 | 1.00 | 57.62 | 8 |
| ATOM | 5401 | N | PHE | C | 239 | 78.194 | 122.951 | 7.648 | 1.00 | 50.91 | 7 |
| ATOM | 5402 | CA | PHE | C | 239 | 78.203 | 121.734 | 6.844 | 1.00 | 52.69 | 6 |
| ATOM | 5403 | CB | PHE | C | 239 | 78.419 | 122.071 | 5.367 | 1.00 | 84.65 | 6 |
| ATOM | 5404 | CG | PHE | C | 239 | 77.904 | 121.022 | 4.414 | 1.00 | 84.64 | 6 |
| ATOM | 5405 | CD1 | PHE | C | 239 | 76.800 | 121.287 | 3.594 | 1.00 | 85.15 | 6 |
| ATOM | 5406 | CD2 | PHE | C | 239 | 78.509 | 119.773 | 4.333 | 1.00 | 83.76 | 6 |
| ATOM | 5407 | CE1 | PHE | C | 239 | 76.307 | 120.323 | 2.707 | 1.00 | 85.25 | 6 |
| ATOM | 5408 | CE2 | PHE | C | 239 | 78.025 | 118.799 | 3.450 | 1.00 | 83.75 | 6 |
| ATOM | 5409 | CZ | PHE | C | 239 | 76.920 | 119.076 | 2.636 | 1.00 | 84.76 | 6 |
| ATOM | 5410 | C | PHE | C | 239 | 79.358 | 120.871 | 7.342 | 1.00 | 54.23 | 6 |
| ATOM | 5411 | O | PHE | C | 239 | 79.191 | 119.690 | 7.643 | 1.00 | 53.69 | 8 |
| ATOM | 5412 | N | THR | C | 240 | 80.531 | 121.486 | 7.441 | 1.00 | 49.83 | 7 |
| ATOM | 5413 | CA | THR | C | 240 | 81.729 | 120.801 | 7.891 | 1.00 | 53.38 | 6 |
| ATOM | 5414 | CB | THR | C | 240 | 82.966 | 121.696 | 7.735 | 1.00 | 75.67 | 6 |
| ATOM | 5415 | OG1 | THR | C | 240 | 83.025 | 122.197 | 6.392 | 1.00 | 76.24 | 8 |
| ATOM | 5416 | CG2 | THR | C | 240 | 84.234 | 120.906 | 8.016 | 1.00 | 75.21 | 6 |
| ATOM | 5417 | C | THR | C | 240 | 81.583 | 120.389 | 9.344 | 1.00 | 56.09 | 6 |
| ATOM | 5418 | O | THR | C | 240 | 82.509 | 119.860 | 9.945 | 1.00 | 56.86 | 8 |
| ATOM | 5419 | N | LEU | C | 241 | 80.412 | 120.642 | 9.912 | 1.00 | 88.04 | 7 |
| ATOM | 5420 | CA | LEU | C | 241 | 80.148 | 120.260 | 11.290 | 1.00 | 92.43 | 6 |
| ATOM | 5421 | CB | LEU | C | 241 | 79.587 | 121.432 | 12.087 | 1.00 | 69.60 | 6 |
| ATOM | 5422 | CG | LEU | C | 241 | 80.587 | 122.299 | 12.847 | 1.00 | 68.57 | 6 |
| ATOM | 5423 | CD1 | LEU | C | 241 | 79.844 | 123.466 | 13.468 | 1.00 | 68.16 | 6 |
| ATOM | 5424 | CD2 | LEU | C | 241 | 81.283 | 121.469 | 13.917 | 1.00 | 68.18 | 6 |
| ATOM | 5425 | C | LEU | C | 241 | 79.156 | 119.113 | 11.286 | 1.00 | 96.01 | 6 |
| ATOM | 5426 | O | LEU | C | 241 | 78.653 | 118.698 | 12.333 | 1.00 | 96.09 | 8 |
| ATOM | 5427 | N | LEU | C | 242 | 78.873 | 118.623 | 10.083 | 1.00 | 88.50 | 7 |
| ATOM | 5428 | CA | LEU | C | 242 | 77.969 | 117.499 | 9.881 | 1.00 | 92.67 | 6 |
| ATOM | 5429 | CB | LEU | C | 242 | 76.785 | 117.901 | 8.990 | 1.00 | 161.63 | 6 |
| ATOM | 5430 | CG | LEU | C | 242 | 75.503 | 118.336 | 9.717 | 1.00 | 162.36 | 6 |
| ATOM | 5431 | CD1 | LEU | C | 242 | 75.769 | 119.543 | 10.604 | 1.00 | 162.25 | 6 |
| ATOM | 5432 | CD2 | LEU | C | 242 | 74.425 | 118.650 | 8.692 | 1.00 | 162.82 | 6 |
| ATOM | 5433 | C | LEU | C | 242 | 78.739 | 116.336 | 9.255 | 1.00 | 95.23 | 6 |
| ATOM | 5434 | O | LEU | C | 242 | 79.088 | 116.353 | 8.070 | 1.00 | 96.06 | 8 |
| ATOM | 5435 | N | ARG | C | 243 | 79.011 | 115.340 | 10.090 | 1.00 | 208.87 | 7 |
| ATOM | 5436 | CA | ARG | C | 243 | 79.737 | 114.140 | 9.700 | 1.00 | 208.87 | 6 |
| ATOM | 5437 | CB | ARG | C | 243 | 79.738 | 113.152 | 10.874 | 1.00 | 203.59 | 6 |
| ATOM | 5438 | CG | ARG | C | 243 | 80.592 | 111.913 | 10.686 | 1.00 | 206.59 | 6 |
| ATOM | 5439 | CD | ARG | C | 243 | 80.534 | 111.041 | 11.922 | 1.00 | 208.78 | 6 |
| ATOM | 5440 | NE | ARG | C | 243 | 81.468 | 109.924 | 11.856 | 1.00 | 208.87 | 7 |
| ATOM | 5441 | CZ | ARG | C | 243 | 81.642 | 109.043 | 12.836 | 1.00 | 208.87 | 6 |
| ATOM | 5442 | NH1 | ARG | C | 243 | 80.942 | 109.152 | 13.958 | 1.00 | 208.87 | 7 |
| ATOM | 5443 | NH2 | ARG | C | 243 | 82.519 | 108.057 | 12.699 | 1.00 | 208.87 | 7 |
| ATOM | 5444 | C | ARG | C | 243 | 79.086 | 113.497 | 8.477 | 1.00 | 208.87 | 6 |
| ATOM | 5445 | O | ARG | C | 243 | 77.873 | 113.592 | 8.289 | 1.00 | 208.87 | 8 |
| ATOM | 5446 | N | PRO | C | 244 | 79.890 | 112.842 | 7.621 | 1.00 | 168.13 | 7 |
| ATOM | 5447 | CD | PRO | C | 244 | 79.366 | 111.947 | 6.570 | 1.00 | 208.87 | 6 |
| ATOM | 5448 | CA | PRO | C | 244 | 81.341 | 112.685 | 7.754 | 1.00 | 167.55 | 6 |
| ATOM | 5449 | CB | PRO | C | 244 | 81.594 | 111.349 | 7.073 | 1.00 | 208.87 | 6 |
| ATOM | 5450 | CG | PRO | C | 244 | 80.636 | 111.410 | 5.925 | 1.00 | 208.87 | 6 |
| ATOM | 5451 | C | PRO | C | 244 | 82.105 | 113.829 | 7.092 | 1.00 | 167.24 | 6 |
| ATOM | 5452 | O | PRO | C | 244 | 81.603 | 114.949 | 6.984 | 1.00 | 167.12 | 8 |
| ATOM | 5453 | N | GLY | C | 245 | 83.321 | 113.533 | 6.649 | 1.00 | 141.10 | 7 |
| ATOM | 5454 | CA | GLY | C | 245 | 84.130 | 114.541 | 5.997 | 1.00 | 140.05 | 6 |
| ATOM | 5455 | C | GLY | C | 245 | 83.762 | 114.705 | 4.536 | 1.00 | 138.99 | 6 |
| ATOM | 5456 | O | GLY | C | 245 | 84.517 | 114.311 | 3.649 | 1.00 | 139.36 | 8 |
| ATOM | 5457 | N | ASP | C | 246 | 82.587 | 115.270 | 4.282 | 1.00 | 91.31 | 7 |
| ATOM | 5458 | CA | ASP | C | 246 | 82.136 | 115.501 | 2.915 | 1.00 | 90.57 | 6 |
| ATOM | 5459 | CB | ASP | C | 246 | 80.625 | 115.749 | 2.900 | 1.00 | 180.76 | 6 |
| ATOM | 5460 | CG | ASP | C | 246 | 79.827 | 114.481 | 3.112 | 1.00 | 181.48 | 6 |
| ATOM | 5461 | OD1 | ASP | C | 246 | 80.029 | 113.518 | 2.343 | 1.00 | 181.22 | 8 |
| ATOM | 5462 | OD2 | ASP | C | 246 | 78.993 | 114.449 | 4.041 | 1.00 | 181.84 | 8 |
| ATOM | 5463 | C | ASP | C | 246 | 82.883 | 116.710 | 2.339 | 1.00 | 89.74 | 6 |
| ATOM | 5464 | O | ASP | C | 246 | 83.858 | 117.180 | 2.927 | 1.00 | 90.14 | 8 |
| ATOM | 5465 | N | PRO | C | 247 | 82.446 | 117.227 | 1.182 | 1.00 | 114.04 | 7 |
| ATOM | 5466 | CD | PRO | C | 247 | 81.519 | 116.670 | 0.181 | 1.00 | 194.03 | 6 |
| ATOM | 5467 | CA | PRO | C | 247 | 83.163 | 118.382 | 0.634 | 1.00 | 113.17 | 6 |
| ATOM | 5468 | CB | PRO | C | 247 | 82.840 | 118.302 | −0.852 | 1.00 | 194.32 | 6 |
| ATOM | 5469 | CG | PRO | C | 247 | 81.437 | 117.785 | −0.839 | 1.00 | 194.34 | 6 |
| ATOM | 5470 | C | PRO | C | 247 | 82.747 | 119.722 | 1.238 | 1.00 | 111.52 | 6 |
| ATOM | 5471 | O | PRO | C | 247 | 81.607 | 120.157 | 1.066 | 1.00 | 111.58 | 8 |
| ATOM | 5472 | N | PRO | C | 248 | 83.668 | 120.397 | 1.955 | 1.00 | 92.49 | 7 |
| ATOM | 5473 | CD | PRO | C | 248 | 84.997 | 119.930 | 2.389 | 1.00 | 76.77 | 6 |
| ATOM | 5474 | CA | PRO | C | 248 | 83.357 | 121.692 | 2.566 | 1.00 | 90.62 | 6 |
| ATOM | 5475 | CB | PRO | C | 248 | 84.686 | 122.105 | 3.185 | 1.00 | 75.12 | 6 |
| ATOM | 5476 | CG | PRO | C | 248 | 85.268 | 120.801 | 3.596 | 1.00 | 75.76 | 6 |
| ATOM | 5477 | C | PRO | C | 248 | 82.867 | 122.693 | 1.530 | 1.00 | 88.92 | 6 |
| ATOM | 5478 | O | PRO | C | 248 | 82.450 | 123.794 | 1.862 | 1.00 | 89.53 | 8 |
| ATOM | 5479 | N | LYS | C | 249 | 82.917 | 122.301 | 0.267 | 1.00 | 61.79 | 7 |
| ATOM | 5480 | CA | LYS | C | 249 | 82.472 | 123.171 | −0.805 | 1.00 | 59.51 | 6 |
| ATOM | 5481 | CB | LYS | C | 249 | 82.371 | 122.373 | −2.107 | 1.00 | 157.18 | 6 |
| ATOM | 5482 | CG | LYS | C | 249 | 82.144 | 123.214 | −3.355 | 1.00 | 158.15 | 6 |
| ATOM | 5483 | CD | LYS | C | 249 | 82.300 | 122.371 | −4.617 | 1.00 | 157.76 | 6 |
| ATOM | 5484 | CE | LYS | C | 249 | 82.232 | 123.225 | −5.873 | 1.00 | 157.27 | 6 |
| ATOM | 5485 | NZ | LYS | C | 249 | 82.505 | 122.427 | −7.101 | 1.00 | 156.24 | 7 |
| ATOM | 5486 | C | LYS | C | 249 | 81.124 | 123.798 | −0.468 | 1.00 | 58.08 | 6 |
| ATOM | 5487 | O | LYS | C | 249 | 80.211 | 123.108 | −0.023 | 1.00 | 57.68 | 8 |
| ATOM | 5488 | N | LYS | C | 250 | 81.028 | 125.112 | −0.661 | 1.00 | 52.07 | 7 |
| ATOM | 5489 | CA | LYS | C | 250 | 79.811 | 125.902 | −0.424 | 1.00 | 51.13 | 6 |
| ATOM | 5490 | CB | LYS | C | 250 | 80.003 | 127.283 | −1.044 | 1.00 | 84.19 | 6 |
| ATOM | 5491 | CG | LYS | C | 250 | 80.498 | 127.165 | −2.489 | 1.00 | 84.65 | 6 |
| ATOM | 5492 | CD | LYS | C | 250 | 80.484 | 128.462 | −3.273 | 1.00 | 84.80 | 6 |
| ATOM | 5493 | CE | LYS | C | 250 | 80.930 | 128.190 | −4.711 | 1.00 | 84.35 | 6 |
| ATOM | 5494 | NZ | LYS | C | 250 | 80.889 | 129.393 | −5.590 | 1.00 | 85.51 | 7 |
| ATOM | 5495 | C | LYS | C | 250 | 78.621 | 125.230 | −1.113 | 1.00 | 49.81 | 6 |
| ATOM | 5496 | O | LYS | C | 250 | 78.672 | 124.996 | −2.319 | 1.00 | 49.67 | 8 |
| ATOM | 5497 | N | ASP | C | 251 | 77.544 | 124.940 | −0.384 | 1.00 | 208.87 | 7 |
| ATOM | 5498 | CA | ASP | C | 251 | 76.402 | 124.280 | −1.018 | 1.00 | 208.87 | 6 |
| ATOM | 5499 | CB | ASP | C | 251 | 76.330 | 122.816 | −0.574 | 1.00 | 84.58 | 6 |
| ATOM | 5500 | CG | ASP | C | 251 | 76.918 | 121.878 | −1.606 | 1.00 | 84.74 | 6 |
| ATOM | 5501 | OD1 | ASP | C | 251 | 76.399 | 121.850 | −2.741 | 1.00 | 83.90 | 8 |
| ATOM | 5502 | OD2 | ASP | C | 251 | 77.902 | 121.177 | −1.293 | 1.00 | 85.22 | 8 |
| ATOM | 5503 | C | ASP | C | 251 | 75.005 | 124.887 | −0.930 | 1.00 | 208.87 | 6 |
| ATOM | 5504 | O | ASP | C | 251 | 74.339 | 125.020 | −1.956 | 1.00 | 208.87 | 8 |
| ATOM | 5505 | N | LYS | C | 252 | 74.553 | 125.244 | 0.271 | 1.00 | 89.65 | 7 |
| ATOM | 5506 | CA | LYS | C | 252 | 73.211 | 125.805 | 0.443 | 1.00 | 86.82 | 6 |
| ATOM | 5507 | CB | LYS | C | 252 | 72.848 | 126.755 | −0.715 | 1.00 | 69.45 | 6 |
| ATOM | 5508 | CG | LYS | C | 252 | 71.372 | 127.168 | −0.758 | 1.00 | 69.86 | 6 |
| ATOM | 5509 | CD | LYS | C | 252 | 71.070 | 128.283 | −1.777 | 1.00 | 69.45 | 6 |
| ATOM | 5510 | CE | LYS | C | 252 | 69.607 | 128.763 | −1.668 | 1.00 | 69.19 | 6 |
| ATOM | 5511 | NZ | LYS | C | 252 | 69.233 | 129.903 | −2.562 | 1.00 | 67.70 | 7 |
| ATOM | 5512 | C | LYS | C | 252 | 72.217 | 124.648 | 0.495 | 1.00 | 85.53 | 6 |
| ATOM | 5513 | O | LYS | C | 252 | 71.167 | 124.741 | 1.127 | 1.00 | 85.56 | 8 |
| ATOM | 5514 | N | ALA | C | 253 | 72.562 | 123.557 | −0.179 | 1.00 | 104.71 | 7 |
| ATOM | 5515 | CA | ALA | C | 253 | 71.717 | 122.373 | −0.200 | 1.00 | 102.89 | 6 |
| ATOM | 5516 | CB | ALA | C | 253 | 72.163 | 121.442 | −1.294 | 1.00 | 66.25 | 6 |
| ATOM | 5517 | C | ALA | C | 253 | 71.848 | 121.695 | 1.156 | 1.00 | 101.99 | 6 |
| ATOM | 5518 | O | ALA | C | 253 | 72.812 | 121.965 | 1.881 | 1.00 | 103.16 | 8 |
| ATOM | 5519 | N | LEU | C | 254 | 70.898 | 120.814 | 1.486 | 1.00 | 67.47 | 7 |
| ATOM | 5520 | CA | LEU | C | 254 | 70.883 | 120.112 | 2.779 | 1.00 | 64.85 | 6 |
| ATOM | 5521 | CB | LEU | C | 254 | 72.302 | 119.723 | 3.209 | 1.00 | 30.25 | 6 |
| ATOM | 5522 | CG | LEU | C | 254 | 72.776 | 119.888 | 4.649 | 1.00 | 28.08 | 6 |
| ATOM | 5523 | CD1 | LEU | C | 254 | 71.826 | 119.234 | 5.637 | 1.00 | 27.43 | 6 |
| ATOM | 5524 | CD2 | LEU | C | 254 | 74.148 | 119.271 | 4.739 | 1.00 | 27.06 | 6 |
| ATOM | 5525 | C | LEU | C | 254 | 70.292 | 121.095 | 3.760 | 1.00 | 64.57 | 6 |
| ATOM | 5526 | O | LEU | C | 254 | 69.409 | 120.749 | 4.542 | 1.00 | 64.88 | 8 |
| ATOM | 5527 | N | ALA | C | 255 | 70.823 | 122.318 | 3.710 | 1.00 | 46.43 | 7 |
| ATOM | 5528 | CA | ALA | C | 255 | 70.357 | 123.440 | 4.510 | 1.00 | 45.16 | 6 |
| ATOM | 5529 | CB | ALA | C | 255 | 71.494 | 124.433 | 4.786 | 1.00 | 13.87 | 6 |
| ATOM | 5530 | C | ALA | C | 255 | 69.338 | 124.024 | 3.539 | 1.00 | 45.33 | 6 |
| ATOM | 5531 | O | ALA | C | 255 | 68.816 | 125.113 | 3.713 | 1.00 | 46.26 | 8 |
| ATOM | 5532 | N | TYR | C | 256 | 69.089 | 123.253 | 2.490 | 1.00 | 49.19 | 7 |
| ATOM | 5533 | CA | TYR | C | 256 | 68.123 | 123.574 | 1.455 | 1.00 | 49.69 | 6 |
| ATOM | 5534 | CB | TYR | C | 256 | 68.525 | 122.884 | 0.152 | 1.00 | 113.70 | 6 |
| ATOM | 5535 | CG | TYR | C | 256 | 68.211 | 123.616 | −1.137 | 1.00 | 116.52 | 6 |
| ATOM | 5536 | CD1 | TYR | C | 256 | 69.006 | 124.680 | −1.570 | 1.00 | 117.43 | 6 |
| ATOM | 5537 | CE1 | TYR | C | 256 | 68.790 | 125.288 | −2.814 | 1.00 | 117.60 | 6 |
| ATOM | 5538 | CD2 | TYR | C | 256 | 67.179 | 123.183 | −1.974 | 1.00 | 117.49 | 6 |
| ATOM | 5539 | CE2 | TYR | C | 256 | 66.952 | 123.783 | −3.219 | 1.00 | 117.99 | 6 |
| ATOM | 5540 | CZ | TYR | C | 256 | 67.764 | 124.832 | −3.635 | 1.00 | 117.79 | 6 |
| ATOM | 5541 | OH | TYR | C | 256 | 67.572 | 125.402 | −4.878 | 1.00 | 116.97 | 8 |
| ATOM | 5542 | C | TYR | C | 256 | 66.885 | 122.887 | 2.013 | 1.00 | 49.09 | 6 |
| ATOM | 5543 | O | TYR | C | 256 | 65.848 | 122.828 | 1.369 | 1.00 | 48.84 | 8 |
| ATOM | 5544 | N | LEU | C | 257 | 67.043 | 122.339 | 3.216 | 1.00 | 77.53 | 7 |
| ATOM | 5545 | CA | LEU | C | 257 | 66.002 | 121.617 | 3.947 | 1.00 | 77.28 | 6 |
| ATOM | 5546 | CB | LEU | C | 257 | 66.437 | 120.174 | 4.204 | 1.00 | 116.17 | 6 |
| ATOM | 5547 | CG | LEU | C | 257 | 66.891 | 119.307 | 3.028 | 1.00 | 117.61 | 6 |
| ATOM | 5548 | CD1 | LEU | C | 257 | 67.935 | 118.302 | 3.512 | 1.00 | 118.96 | 6 |
| ATOM | 5549 | CD2 | LEU | C | 257 | 65.683 | 118.612 | 2.400 | 1.00 | 116.94 | 6 |
| ATOM | 5550 | C | LEU | C | 257 | 65.889 | 122.323 | 5.281 | 1.00 | 76.11 | 6 |
| ATOM | 5551 | O | LEU | C | 257 | 65.799 | 123.544 | 5.314 | 1.00 | 76.74 | 8 |
| ATOM | 5552 | N | PHE | C | 258 | 65.934 | 121.543 | 6.366 | 1.00 | 66.70 | 7 |
| ATOM | 5553 | CA | PHE | C | 258 | 65.856 | 122.057 | 7.739 | 1.00 | 65.52 | 6 |
| ATOM | 5554 | CB | PHE | C | 258 | 67.227 | 122.053 | 8.407 | 1.00 | 60.51 | 6 |
| ATOM | 5555 | CG | PHE | C | 258 | 67.903 | 120.721 | 8.405 | 1.00 | 61.13 | 6 |
| ATOM | 5556 | CD1 | PHE | C | 258 | 68.146 | 120.052 | 7.216 | 1.00 | 60.44 | 6 |
| ATOM | 5557 | CD2 | PHE | C | 258 | 68.352 | 120.159 | 9.594 | 1.00 | 61.98 | 6 |
| ATOM | 5558 | CE1 | PHE | C | 258 | 68.829 | 118.853 | 7.213 | 1.00 | 60.93 | 6 |
| ATOM | 5559 | CE2 | PHE | C | 258 | 69.041 | 118.954 | 9.601 | 1.00 | 61.63 | 6 |
| ATOM | 5560 | CZ | PHE | C | 258 | 69.280 | 118.302 | 8.413 | 1.00 | 61.42 | 6 |
| ATOM | 5561 | C | PHE | C | 258 | 65.349 | 123.479 | 7.735 | 1.00 | 65.21 | 6 |
| ATOM | 5562 | O | PHE | C | 258 | 65.923 | 124.359 | 8.385 | 1.00 | 65.27 | 8 |
| ATOM | 5563 | N | GLY | C | 259 | 64.269 | 123.685 | 6.990 | 1.00 | 42.12 | 7 |
| ATOM | 5564 | CA | GLY | C | 259 | 63.679 | 124.995 | 6.850 | 1.00 | 40.93 | 6 |
| ATOM | 5565 | C | GLY | C | 259 | 63.238 | 125.119 | 5.408 | 1.00 | 40.90 | 6 |
| ATOM | 5566 | O | GLY | C | 259 | 62.037 | 125.170 | 5.129 | 1.00 | 40.65 | 8 |
| ATOM | 5567 | N | LEU | C | 260 | 64.200 | 125.147 | 4.485 | 1.00 | 31.09 | 7 |
| ATOM | 5568 | CA | LEU | C | 260 | 63.864 | 125.273 | 3.067 | 1.00 | 32.91 | 6 |
| ATOM | 5569 | CB | LEU | C | 260 | 65.137 | 125.288 | 2.195 | 1.00 | 54.75 | 6 |
| ATOM | 5570 | CG | LEU | C | 260 | 65.062 | 125.650 | 0.688 | 1.00 | 53.93 | 6 |
| ATOM | 5571 | CD1 | LEU | C | 260 | 66.449 | 125.979 | 0.197 | 1.00 | 54.88 | 6 |
| ATOM | 5572 | CD2 | LEU | C | 260 | 64.503 | 124.532 | −0.167 | 1.00 | 53.12 | 6 |
| ATOM | 5573 | C | LEU | C | 260 | 62.942 | 124.139 | 2.627 | 1.00 | 33.56 | 6 |
| ATOM | 5574 | O | LEU | C | 260 | 62.855 | 123.107 | 3.286 | 1.00 | 32.20 | 8 |
| ATOM | 5575 | N | LEU | C | 261 | 62.252 | 124.360 | 1.514 | 1.00 | 74.26 | 7 |
| ATOM | 5576 | CA | LEU | C | 261 | 61.329 | 123.396 | 0.923 | 1.00 | 76.02 | 6 |
| ATOM | 5577 | CB | LEU | C | 261 | 62.083 | 122.408 | 0.015 | 1.00 | 80.87 | 6 |
| ATOM | 5578 | CG | LEU | C | 261 | 62.573 | 121.041 | 0.505 | 1.00 | 80.48 | 6 |
| ATOM | 5579 | CD1 | LEU | C | 261 | 63.112 | 120.264 | −0.688 | 1.00 | 80.79 | 6 |
| ATOM | 5580 | CD2 | LEU | C | 261 | 63.641 | 121.187 | 1.559 | 1.00 | 80.51 | 6 |
| ATOM | 5581 | C | LEU | C | 261 | 60.452 | 122.621 | 1.894 | 1.00 | 77.26 | 6 |
| ATOM | 5582 | O | LEU | C | 261 | 60.928 | 121.854 | 2.729 | 1.00 | 76.99 | 8 |
| ATOM | 5583 | N | ALA | C | 262 | 59.150 | 122.829 | 1.751 | 1.00 | 44.61 | 7 |
| ATOM | 5584 | CA | ALA | C | 262 | 58.168 | 122.171 | 2.580 | 1.00 | 45.54 | 6 |
| ATOM | 5585 | CB | ALA | C | 262 | 56.803 | 122.666 | 2.190 | 1.00 | 13.87 | 6 |
| ATOM | 5586 | C | ALA | C | 262 | 58.260 | 120.654 | 2.404 | 1.00 | 47.51 | 6 |
| ATOM | 5587 | O | ALA | C | 262 | 57.279 | 119.934 | 2.626 | 1.00 | 48.30 | 8 |
| ATOM | 5588 | N | ASP | C | 263 | 59.439 | 120.171 | 2.009 | 1.00 | 48.36 | 7 |
| ATOM | 5589 | CA | ASP | C | 263 | 59.652 | 118.743 | 1.785 | 1.00 | 51.39 | 6 |
| ATOM | 5590 | CB | ASP | C | 263 | 60.790 | 118.525 | 0.783 | 1.00 | 112.70 | 6 |
| ATOM | 5591 | CG | ASP | C | 263 | 61.786 | 117.462 | 1.243 | 1.00 | 114.08 | 6 |
| ATOM | 5592 | OD1 | ASP | C | 263 | 61.369 | 116.302 | 1.432 | 1.00 | 115.21 | 8 |
| ATOM | 5593 | OD2 | ASP | C | 263 | 62.982 | 117.786 | 1.422 | 1.00 | 113.92 | 8 |
| ATOM | 5594 | C | ASP | C | 263 | 59.941 | 117.941 | 3.043 | 1.00 | 52.97 | 6 |
| ATOM | 5595 | O | ASP | C | 263 | 59.361 | 116.875 | 3.240 | 1.00 | 52.61 | 8 |
| ATOM | 5596 | N | PRO | C | 264 | 60.864 | 118.421 | 3.893 | 1.00 | 61.10 | 7 |
| ATOM | 5597 | CD | PRO | C | 264 | 61.992 | 119.306 | 3.555 | 1.00 | 121.93 | 6 |
| ATOM | 5598 | CA | PRO | C | 264 | 61.175 | 117.681 | 5.114 | 1.00 | 63.87 | 6 |
| ATOM | 5599 | CB | PRO | C | 264 | 62.656 | 117.405 | 4.942 | 1.00 | 123.85 | 6 |
| ATOM | 5600 | CG | PRO | C | 264 | 63.142 | 118.742 | 4.415 | 1.00 | 123.04 | 6 |
| ATOM | 5601 | C | PRO | C | 264 | 60.885 | 118.496 | 6.377 | 1.00 | 65.94 | 6 |
| ATOM | 5602 | O | PRO | C | 264 | 61.380 | 118.184 | 7.465 | 1.00 | 65.59 | 8 |
| ATOM | 5603 | N | LYS | C | 265 | 60.082 | 119.542 | 6.232 | 1.00 | 114.70 | 7 |
| ATOM | 5604 | CA | LYS | C | 265 | 59.762 | 120.389 | 7.370 | 1.00 | 117.68 | 6 |
| ATOM | 5605 | CB | LYS | C | 265 | 60.964 | 121.290 | 7.703 | 1.00 | 69.15 | 6 |
| ATOM | 5606 | CG | LYS | C | 265 | 60.841 | 122.110 | 8.987 | 1.00 | 68.89 | 6 |
| ATOM | 5607 | CD | LYS | C | 265 | 61.275 | 121.307 | 10.208 | 1.00 | 69.00 | 6 |
| ATOM | 5608 | CE | LYS | C | 265 | 61.219 | 122.141 | 11.486 | 1.00 | 69.27 | 6 |
| ATOM | 5609 | NZ | LYS | C | 265 | 61.710 | 121.388 | 12.683 | 1.00 | 69.17 | 7 |
| ATOM | 5610 | C | LYS | C | 265 | 58.551 | 121.253 | 7.062 | 1.00 | 120.01 | 6 |
| ATOM | 5611 | O | LYS | C | 265 | 58.690 | 122.360 | 6.555 | 1.00 | 119.67 | 8 |
| ATOM | 5612 | N | ARG | C | 266 | 57.361 | 120.744 | 7.345 | 1.00 | 80.91 | 7 |
| ATOM | 5613 | CA | ARG | C | 266 | 56.165 | 121.534 | 7.118 | 1.00 | 84.78 | 6 |
| ATOM | 5614 | CB | ARG | C | 266 | 55.942 | 121.783 | 5.618 | 1.00 | 126.80 | 6 |
| ATOM | 5615 | CG | ARG | C | 266 | 54.875 | 120.918 | 4.943 | 1.00 | 129.36 | 6 |
| ATOM | 5616 | CD | ARG | C | 266 | 54.358 | 121.611 | 3.679 | 1.00 | 131.03 | 6 |
| ATOM | 5617 | NE | ARG | C | 266 | 53.368 | 120.833 | 2.937 | 1.00 | 132.53 | 7 |
| ATOM | 5618 | CZ | ARG | C | 266 | 52.662 | 121.309 | 1.913 | 1.00 | 133.01 | 6 |
| ATOM | 5619 | NH1 | ARG | C | 266 | 52.830 | 122.562 | 1.506 | 1.00 | 132.55 | 7 |
| ATOM | 5620 | NH2 | ARG | C | 266 | 51.783 | 120.534 | 1.295 | 1.00 | 134.02 | 7 |
| ATOM | 5621 | C | ARG | C | 266 | 54.921 | 120.928 | 7.740 | 1.00 | 86.59 | 6 |
| ATOM | 5622 | O | ARG | C | 266 | 54.568 | 119.779 | 7.482 | 1.00 | 86.90 | 8 |
| ATOM | 5623 | N | TYR | C | 267 | 54.265 | 121.726 | 8.574 | 1.00 | 163.69 | 7 |
| ATOM | 5624 | CA | TYR | C | 267 | 53.044 | 121.312 | 9.244 | 1.00 | 165.06 | 6 |
| ATOM | 5625 | CB | TYR | C | 267 | 52.944 | 121.975 | 10.629 | 1.00 | 86.31 | 6 |
| ATOM | 5626 | CG | TYR | C | 267 | 54.289 | 122.359 | 11.228 | 1.00 | 86.21 | 6 |
| ATOM | 5627 | CD1 | TYR | C | 267 | 54.387 | 123.415 | 12.128 | 1.00 | 87.05 | 6 |
| ATOM | 5628 | CE1 | TYR | C | 267 | 55.630 | 123.863 | 12.590 | 1.00 | 86.89 | 6 |
| ATOM | 5629 | CD2 | TYR | C | 267 | 55.472 | 121.743 | 10.815 | 1.00 | 85.28 | 6 |
| ATOM | 5630 | CE2 | TYR | C | 267 | 56.713 | 122.182 | 11.267 | 1.00 | 85.79 | 6 |
| ATOM | 5631 | CZ | TYR | C | 267 | 56.785 | 123.245 | 12.151 | 1.00 | 86.10 | 6 |
| ATOM | 5632 | OH | TYR | C | 267 | 58.007 | 123.714 | 12.572 | 1.00 | 85.99 | 8 |
| ATOM | 5633 | C | TYR | C | 267 | 51.975 | 121.834 | 8.286 | 1.00 | 165.93 | 6 |
| ATOM | 5634 | O | TYR | C | 267 | 50.838 | 122.116 | 8.669 | 1.00 | 166.25 | 8 |
| ATOM | 5635 | N | ASP | C | 268 | 52.406 | 121.959 | 7.030 | 1.00 | 129.89 | 7 |
| ATOM | 5636 | CA | ASP | C | 268 | 51.617 | 122.416 | 5.888 | 1.00 | 131.45 | 6 |
| ATOM | 5637 | CB | ASP | C | 268 | 51.233 | 121.210 | 5.003 | 1.00 | 182.27 | 6 |
| ATOM | 5638 | CG | ASP | C | 268 | 50.760 | 119.998 | 5.806 | 1.00 | 183.34 | 6 |
| ATOM | 5639 | OD1 | ASP | C | 268 | 51.580 | 119.405 | 6.540 | 1.00 | 183.74 | 8 |
| ATOM | 5640 | OD2 | ASP | C | 268 | 49.568 | 119.633 | 5.694 | 1.00 | 184.62 | 8 |
| ATOM | 5641 | C | ASP | C | 268 | 50.382 | 123.275 | 6.142 | 1.00 | 131.54 | 6 |
| ATOM | 5642 | O | ASP | C | 268 | 50.344 | 124.063 | 7.089 | 1.00 | 131.27 | 8 |
| ATOM | 5643 | N | ALA | C | 269 | 49.399 | 123.121 | 5.247 | 1.00 | 198.05 | 7 |
| ATOM | 5644 | CA | ALA | C | 269 | 48.109 | 123.819 | 5.275 | 1.00 | 197.81 | 6 |
| ATOM | 5645 | CB | ALA | C | 269 | 47.081 | 122.971 | 6.053 | 1.00 | 84.50 | 6 |
| ATOM | 5646 | C | ALA | C | 269 | 48.179 | 125.228 | 5.856 | 1.00 | 197.19 | 6 |
| ATOM | 5647 | O | ALA | C | 269 | 48.035 | 126.228 | 5.150 | 1.00 | 197.51 | 8 |
| ATOM | 5648 | N | GLY | C | 270 | 48.391 | 125.284 | 7.160 | 1.00 | 123.16 | 7 |
| ATOM | 5649 | CA | GLY | C | 270 | 48.487 | 126.544 | 7.854 | 1.00 | 121.84 | 6 |
| ATOM | 5650 | C | GLY | C | 270 | 48.242 | 126.229 | 9.311 | 1.00 | 121.58 | 6 |
| ATOM | 5651 | O | GLY | C | 270 | 48.893 | 126.770 | 10.200 | 1.00 | 121.86 | 8 |
| ATOM | 5652 | N | GLU | C | 271 | 47.312 | 125.314 | 9.548 | 1.00 | 56.86 | 7 |
| ATOM | 5653 | CA | GLU | C | 271 | 46.955 | 124.928 | 10.900 | 1.00 | 56.02 | 6 |
| ATOM | 5654 | CB | GLU | C | 271 | 45.659 | 124.117 | 10.870 | 1.00 | 103.22 | 6 |
| ATOM | 5655 | CG | GLU | C | 271 | 44.493 | 124.851 | 10.224 | 1.00 | 102.91 | 6 |
| ATOM | 5656 | CD | GLU | C | 271 | 44.210 | 126.195 | 10.872 | 1.00 | 103.24 | 6 |
| ATOM | 5657 | OE1 | GLU | C | 271 | 43.798 | 126.221 | 12.052 | 1.00 | 103.46 | 8 |
| ATOM | 5658 | OE2 | GLU | C | 271 | 44.399 | 127.226 | 10.196 | 1.00 | 103.93 | 8 |
| ATOM | 5659 | C | GLU | C | 271 | 48.040 | 124.144 | 11.635 | 1.00 | 55.94 | 6 |
| ATOM | 5660 | O | GLU | C | 271 | 48.949 | 123.584 | 11.020 | 1.00 | 55.00 | 8 |
| ATOM | 5661 | N | ALA | C | 272 | 47.925 | 124.145 | 12.963 | 1.00 | 136.30 | 7 |
| ATOM | 5662 | CA | ALA | C | 272 | 48.822 | 123.445 | 13.883 | 1.00 | 137.12 | 6 |
| ATOM | 5663 | CB | ALA | C | 272 | 48.819 | 121.943 | 13.563 | 1.00 | 70.85 | 6 |
| ATOM | 5664 | C | ALA | C | 272 | 50.265 | 123.957 | 13.996 | 1.00 | 138.00 | 6 |
| ATOM | 5665 | O | ALA | C | 272 | 51.048 | 123.432 | 14.792 | 1.00 | 138.36 | 8 |
| ATOM | 5666 | N | GLY | C | 273 | 50.622 | 124.980 | 13.225 | 1.00 | 107.29 | 7 |
| ATOM | 5667 | CA | GLY | C | 273 | 51.981 | 125.498 | 13.293 | 1.00 | 107.57 | 6 |
| ATOM | 5668 | C | GLY | C | 273 | 52.072 | 127.006 | 13.427 | 1.00 | 107.38 | 6 |
| ATOM | 5669 | O | GLY | C | 273 | 52.775 | 127.532 | 14.290 | 1.00 | 106.84 | 8 |
| ATOM | 5670 | N | ARG | C | 274 | 51.371 | 127.708 | 12.548 | 1.00 | 163.77 | 7 |
| ATOM | 5671 | CA | ARG | C | 274 | 51.368 | 129.155 | 12.597 | 1.00 | 164.91 | 6 |
| ATOM | 5672 | CB | ARG | C | 274 | 50.629 | 129.729 | 11.382 | 1.00 | 130.45 | 6 |
| ATOM | 5673 | CG | ARG | C | 274 | 49.139 | 129.418 | 11.330 | 1.00 | 130.94 | 6 |
| ATOM | 5674 | CD | ARG | C | 274 | 48.559 | 129.727 | 9.960 | 1.00 | 131.16 | 6 |
| ATOM | 5675 | NE | ARG | C | 274 | 47.121 | 129.490 | 9.909 | 1.00 | 132.12 | 7 |
| ATOM | 5676 | CZ | ARG | C | 274 | 46.406 | 129.467 | 8.788 | 1.00 | 132.41 | 6 |
| ATOM | 5677 | NH1 | ARG | C | 274 | 46.998 | 129.664 | 7.614 | 1.00 | 131.64 | 7 |
| ATOM | 5678 | NH2 | ARG | C | 274 | 45.098 | 129.252 | 8.842 | 1.00 | 133.00 | 7 |
| ATOM | 5679 | C | ARG | C | 274 | 50.648 | 129.516 | 13.886 | 1.00 | 165.08 | 6 |
| ATOM | 5680 | O | ARG | C | 274 | 51.175 | 130.255 | 14.717 | 1.00 | 165.31 | 8 |
| ATOM | 5681 | N | TYR | C | 275 | 49.450 | 128.965 | 14.055 | 1.00 | 126.35 | 7 |
| ATOM | 5682 | CA | TYR | C | 275 | 48.652 | 129.214 | 15.243 | 1.00 | 127.24 | 6 |
| ATOM | 5683 | CB | TYR | C | 275 | 47.471 | 128.246 | 15.304 | 1.00 | 208.45 | 6 |
| ATOM | 5684 | CG | TYR | C | 275 | 46.313 | 128.627 | 14.412 | 1.00 | 208.87 | 6 |
| ATOM | 5685 | CD1 | TYR | C | 275 | 46.488 | 128.794 | 13.040 | 1.00 | 208.87 | 6 |
| ATOM | 5686 | CE1 | TYR | C | 275 | 45.421 | 129.137 | 12.213 | 1.00 | 208.87 | 6 |
| ATOM | 5687 | CD2 | TYR | C | 275 | 45.036 | 128.812 | 14.940 | 1.00 | 208.87 | 6 |
| ATOM | 5688 | CE2 | TYR | C | 275 | 43.960 | 129.154 | 14.122 | 1.00 | 208.87 | 6 |
| ATOM | 5689 | CZ | TYR | C | 275 | 44.160 | 129.315 | 12.759 | 1.00 | 208.87 | 6 |
| ATOM | 5690 | OH | TYR | C | 275 | 43.099 | 129.642 | 11.942 | 1.00 | 208.87 | 8 |
| ATOM | 5691 | C | TYR | C | 275 | 49.509 | 129.044 | 16.482 | 1.00 | 127.57 | 6 |
| ATOM | 5692 | O | TYR | C | 275 | 49.534 | 129.906 | 17.358 | 1.00 | 128.16 | 8 |
| ATOM | 5693 | N | ALA | C | 276 | 50.225 | 127.932 | 16.550 | 1.00 | 62.61 | 7 |
| ATOM | 5694 | CA | ALA | C | 276 | 51.067 | 127.683 | 17.703 | 1.00 | 61.43 | 6 |
| ATOM | 5695 | CB | ALA | C | 276 | 51.898 | 126.435 | 17.470 | 1.00 | 160.70 | 6 |
| ATOM | 5696 | C | ALA | C | 276 | 51.977 | 128.872 | 18.019 | 1.00 | 60.68 | 6 |
| ATOM | 5697 | O | ALA | C | 276 | 51.614 | 129.755 | 18.804 | 1.00 | 60.70 | 8 |
| ATOM | 5698 | N | ALA | C | 277 | 53.155 | 128.882 | 17.395 | 1.00 | 41.08 | 7 |
| ATOM | 5699 | CA | ALA | C | 277 | 54.162 | 129.925 | 17.587 | 1.00 | 40.08 | 6 |
| ATOM | 5700 | CB | ALA | C | 277 | 55.221 | 129.831 | 16.495 | 1.00 | 110.85 | 6 |
| ATOM | 5701 | C | ALA | C | 277 | 53.583 | 131.328 | 17.613 | 1.00 | 39.89 | 6 |
| ATOM | 5702 | O | ALA | C | 277 | 53.900 | 132.111 | 18.506 | 1.00 | 38.90 | 8 |
| ATOM | 5703 | N | GLU | C | 278 | 52.739 | 131.644 | 16.633 | 1.00 | 158.16 | 7 |
| ATOM | 5704 | CA | GLU | C | 278 | 52.138 | 132.970 | 16.553 | 1.00 | 159.25 | 6 |
| ATOM | 5705 | CB | GLU | C | 278 | 51.176 | 133.072 | 15.363 | 1.00 | 206.63 | 6 |
| ATOM | 5706 | CG | GLU | C | 278 | 50.658 | 134.491 | 15.119 | 1.00 | 208.87 | 6 |
| ATOM | 5707 | CD | GLU | C | 278 | 49.767 | 134.604 | 13.893 | 1.00 | 208.87 | 6 |
| ATOM | 5708 | OE1 | GLU | C | 278 | 48.678 | 133.994 | 13.882 | 1.00 | 208.87 | 8 |
| ATOM | 5709 | OE2 | GLU | C | 278 | 50.158 | 135.306 | 12.936 | 1.00 | 208.87 | 8 |
| ATOM | 5710 | C | GLU | C | 278 | 51.398 | 133.302 | 17.834 | 1.00 | 158.42 | 6 |
| ATOM | 5711 | O | GLU | C | 278 | 51.175 | 134.468 | 18.147 | 1.00 | 158.85 | 8 |
| ATOM | 5712 | N | GLU | C | 279 | 51.007 | 132.278 | 18.576 | 1.00 | 95.46 | 7 |
| ATOM | 5713 | CA | GLU | C | 279 | 50.313 | 132.511 | 19.825 | 1.00 | 93.85 | 6 |
| ATOM | 5714 | CB | GLU | C | 279 | 48.905 | 131.927 | 19.770 | 1.00 | 94.99 | 6 |
| ATOM | 5715 | CG | GLU | C | 279 | 47.952 | 132.709 | 18.864 | 1.00 | 95.71 | 6 |
| ATOM | 5716 | CD | GLU | C | 279 | 47.892 | 134.192 | 19.207 | 1.00 | 96.88 | 6 |
| ATOM | 5717 | OE1 | GLU | C | 279 | 47.698 | 134.525 | 20.394 | 1.00 | 97.18 | 8 |
| ATOM | 5718 | OE2 | GLU | C | 279 | 48.031 | 135.033 | 18.292 | 1.00 | 98.81 | 8 |
| ATOM | 5719 | C | GLU | C | 279 | 51.115 | 131.910 | 20.960 | 1.00 | 92.63 | 6 |
| ATOM | 5720 | O | GLU | C | 279 | 50.578 | 131.279 | 21.865 | 1.00 | 93.45 | 8 |
| ATOM | 5721 | N | LYS | C | 280 | 52.420 | 132.124 | 20.885 | 1.00 | 48.25 | 7 |
| ATOM | 5722 | CA | LYS | C | 280 | 53.374 | 131.649 | 21.877 | 1.00 | 47.22 | 6 |
| ATOM | 5723 | CB | LYS | C | 280 | 53.812 | 130.213 | 21.571 | 1.00 | 149.55 | 6 |
| ATOM | 5724 | CG | LYS | C | 280 | 52.707 | 129.162 | 21.590 | 1.00 | 151.23 | 6 |
| ATOM | 5725 | CD | LYS | C | 280 | 52.316 | 128.745 | 23.004 | 1.00 | 151.98 | 6 |
| ATOM | 5726 | CE | LYS | C | 280 | 51.303 | 127.596 | 22.974 | 1.00 | 152.43 | 6 |
| ATOM | 5727 | NZ | LYS | C | 280 | 50.930 | 127.093 | 24.331 | 1.00 | 151.40 | 7 |
| ATOM | 5728 | C | LYS | C | 280 | 54.570 | 132.581 | 21.718 | 1.00 | 46.41 | 6 |
| ATOM | 5729 | O | LYS | C | 280 | 55.041 | 133.204 | 22.673 | 1.00 | 45.83 | 8 |
| ATOM | 5730 | N | LEU | C | 281 | 55.052 | 132.666 | 20.484 | 1.00 | 94.79 | 7 |
| ATOM | 5731 | CA | LEU | C | 281 | 56.181 | 133.514 | 20.143 | 1.00 | 94.93 | 6 |
| ATOM | 5732 | CB | LEU | C | 281 | 56.928 | 132.932 | 18.942 | 1.00 | 69.65 | 6 |
| ATOM | 5733 | CG | LEU | C | 281 | 57.586 | 131.565 | 19.112 | 1.00 | 70.36 | 6 |
| ATOM | 5734 | CD1 | LEU | C | 281 | 58.050 | 131.071 | 17.764 | 1.00 | 69.69 | 6 |
| ATOM | 5735 | CD2 | LEU | C | 281 | 58.744 | 131.665 | 20.082 | 1.00 | 70.93 | 6 |
| ATOM | 5736 | C | LEU | C | 281 | 55.652 | 134.898 | 19.794 | 1.00 | 95.02 | 6 |
| ATOM | 5737 | O | LEU | C | 281 | 54.462 | 135.169 | 19.946 | 1.00 | 94.94 | 8 |
| ATOM | 5738 | N | GLY | C | 282 | 56.543 | 135.764 | 19.322 | 1.00 | 208.87 | 7 |
| ATOM | 5739 | CA | GLY | C | 282 | 56.153 | 137.113 | 18.954 | 1.00 | 208.87 | 6 |
| ATOM | 5740 | C | GLY | C | 282 | 54.832 | 137.162 | 18.213 | 1.00 | 208.87 | 6 |
| ATOM | 5741 | O | GLY | C | 282 | 53.907 | 137.837 | 18.658 | 1.00 | 208.87 | 8 |
| ATOM | 5742 | N | VAL | C | 283 | 54.756 | 136.438 | 17.096 | 1.00 | 107.38 | 7 |
| ATOM | 5743 | CA | VAL | C | 283 | 53.568 | 136.359 | 16.239 | 1.00 | 106.04 | 6 |
| ATOM | 5744 | CB | VAL | C | 283 | 52.587 | 137.562 | 16.457 | 1.00 | 159.21 | 6 |
| ATOM | 5745 | CG1 | VAL | C | 283 | 51.944 | 137.990 | 15.142 | 1.00 | 159.40 | 6 |
| ATOM | 5746 | CG2 | VAL | C | 283 | 51.490 | 137.167 | 17.433 | 1.00 | 158.58 | 6 |
| ATOM | 5747 | C | VAL | C | 283 | 54.040 | 136.358 | 14.795 | 1.00 | 105.26 | 6 |
| ATOM | 5748 | O | VAL | C | 283 | 55.190 | 136.684 | 14.520 | 1.00 | 106.40 | 8 |
| ATOM | 5749 | N | GLY | C | 284 | 53.152 | 135.980 | 13.882 | 1.00 | 79.40 | 7 |
| ATOM | 5750 | CA | GLY | C | 284 | 53.495 | 135.955 | 12.473 | 1.00 | 78.56 | 6 |
| ATOM | 5751 | C | GLY | C | 284 | 54.516 | 134.892 | 12.116 | 1.00 | 78.47 | 6 |
| ATOM | 5752 | O | GLY | C | 284 | 55.423 | 134.604 | 12.898 | 1.00 | 78.70 | 8 |
| ATOM | 5753 | N | LEU | C | 285 | 54.376 | 134.312 | 10.928 | 1.00 | 96.55 | 7 |
| ATOM | 5754 | CA | LEU | C | 285 | 55.292 | 133.272 | 10.475 | 1.00 | 95.68 | 6 |
| ATOM | 5755 | CB | LEU | C | 285 | 55.217 | 132.086 | 11.426 | 1.00 | 67.63 | 6 |
| ATOM | 5756 | CG | LEU | C | 285 | 56.501 | 131.361 | 11.808 | 1.00 | 66.94 | 6 |
| ATOM | 5757 | CD1 | LEU | C | 285 | 56.192 | 130.527 | 13.029 | 1.00 | 66.94 | 6 |
| ATOM | 5758 | CD2 | LEU | C | 285 | 57.019 | 130.489 | 10.669 | 1.00 | 66.84 | 6 |
| ATOM | 5759 | C | LEU | C | 285 | 54.866 | 132.852 | 9.077 | 1.00 | 95.70 | 6 |
| ATOM | 5760 | O | LEU | C | 285 | 54.099 | 133.559 | 8.434 | 1.00 | 95.72 | 8 |
| ATOM | 5761 | N | SER | C | 286 | 55.351 | 131.705 | 8.609 | 1.00 | 140.80 | 7 |
| ATOM | 5762 | CA | SER | C | 286 | 55.001 | 131.229 | 7.274 | 1.00 | 140.03 | 6 |
| ATOM | 5763 | CB | SER | C | 286 | 55.741 | 132.051 | 6.207 | 1.00 | 102.60 | 6 |
| ATOM | 5764 | OG | SER | C | 286 | 55.555 | 133.446 | 6.381 | 1.00 | 101.88 | 8 |
| ATOM | 5765 | C | SER | C | 286 | 55.343 | 129.753 | 7.072 | 1.00 | 139.39 | 6 |
| ATOM | 5766 | O | SER | C | 286 | 56.515 | 129.401 | 6.961 | 1.00 | 139.47 | 8 |
| ATOM | 5767 | N | GLY | C | 287 | 54.322 | 128.900 | 7.028 | 1.00 | 95.77 | 7 |
| ATOM | 5768 | CA | GLY | C | 287 | 54.532 | 127.474 | 6.805 | 1.00 | 95.78 | 6 |
| ATOM | 5769 | C | GLY | C | 287 | 55.666 | 126.736 | 7.510 | 1.00 | 96.06 | 6 |
| ATOM | 5770 | O | GLY | C | 287 | 55.409 | 125.965 | 8.437 | 1.00 | 95.73 | 8 |
| ATOM | 5771 | N | ALA | C | 288 | 56.912 | 126.945 | 7.071 | 1.00 | 94.57 | 7 |
| ATOM | 5772 | CA | ALA | C | 288 | 58.071 | 126.268 | 7.671 | 1.00 | 94.17 | 6 |
| ATOM | 5773 | CB | ALA | C | 288 | 58.667 | 125.298 | 6.676 | 1.00 | 50.77 | 6 |
| ATOM | 5774 | C | ALA | C | 288 | 59.189 | 127.159 | 8.212 | 1.00 | 93.74 | 6 |
| ATOM | 5775 | O | ALA | C | 288 | 59.444 | 128.249 | 7.703 | 1.00 | 93.76 | 8 |
| ATOM | 5776 | N | THR | C | 289 | 59.862 | 126.661 | 9.245 | 1.00 | 94.67 | 7 |
| ATOM | 5777 | CA | THR | C | 289 | 60.980 | 127.362 | 9.869 | 1.00 | 95.18 | 6 |
| ATOM | 5778 | CB | THR | C | 289 | 61.803 | 126.430 | 10.805 | 1.00 | 89.75 | 6 |
| ATOM | 5779 | OG1 | THR | C | 289 | 60.986 | 125.955 | 11.885 | 1.00 | 89.80 | 8 |
| ATOM | 5780 | CG2 | THR | C | 289 | 62.991 | 127.173 | 11.365 | 1.00 | 89.32 | 6 |
| ATOM | 5781 | C | THR | C | 289 | 61.898 | 127.792 | 8.740 | 1.00 | 95.31 | 6 |
| ATOM | 5782 | O | THR | C | 289 | 62.015 | 127.087 | 7.741 | 1.00 | 95.64 | 8 |
| ATOM | 5783 | N | LEU | C | 290 | 62.548 | 128.939 | 8.902 | 1.00 | 55.18 | 7 |
| ATOM | 5784 | CA | LEU | C | 290 | 63.465 | 129.465 | 7.891 | 1.00 | 56.06 | 6 |
| ATOM | 5785 | CB | LEU | C | 290 | 64.782 | 128.695 | 7.932 | 1.00 | 88.40 | 6 |
| ATOM | 5786 | CG | LEU | C | 290 | 65.811 | 129.042 | 6.857 | 1.00 | 88.84 | 6 |
| ATOM | 5787 | CD1 | LEU | C | 290 | 66.107 | 130.527 | 6.877 | 1.00 | 89.92 | 6 |
| ATOM | 5788 | CD2 | LEU | C | 290 | 67.075 | 128.243 | 7.104 | 1.00 | 88.80 | 6 |
| ATOM | 5789 | C | LEU | C | 290 | 62.903 | 129.441 | 6.469 | 1.00 | 56.25 | 6 |
| ATOM | 5790 | O | LEU | C | 290 | 61.992 | 128.682 | 6.146 | 1.00 | 55.02 | 8 |
| ATOM | 5791 | N | VAL | C | 291 | 63.445 | 130.298 | 5.619 | 1.00 | 81.17 | 7 |
| ATOM | 5792 | CA | VAL | C | 291 | 62.994 | 130.365 | 4.243 | 1.00 | 83.23 | 6 |
| ATOM | 5793 | CB | VAL | C | 291 | 61.892 | 131.437 | 4.046 | 1.00 | 129.94 | 6 |
| ATOM | 5794 | CG1 | VAL | C | 291 | 61.529 | 131.544 | 2.574 | 1.00 | 129.79 | 6 |
| ATOM | 5795 | CG2 | VAL | C | 291 | 60.659 | 131.080 | 4.862 | 1.00 | 130.39 | 6 |
| ATOM | 5796 | C | VAL | C | 291 | 64.176 | 130.704 | 3.359 | 1.00 | 84.36 | 6 |
| ATOM | 5797 | O | VAL | C | 291 | 64.829 | 131.734 | 3.536 | 1.00 | 83.65 | 8 |
| ATOM | 5798 | N | ALA | C | 292 | 64.449 | 129.814 | 2.412 | 1.00 | 196.81 | 7 |
| ATOM | 5799 | CA | ALA | C | 292 | 65.545 | 129.991 | 1.475 | 1.00 | 198.79 | 6 |
| ATOM | 5800 | CB | ALA | C | 292 | 66.540 | 128.861 | 1.626 | 1.00 | 125.71 | 6 |
| ATOM | 5801 | C | ALA | C | 292 | 64.990 | 130.017 | 0.058 | 1.00 | 200.14 | 6 |
| ATOM | 5802 | O | ALA | C | 292 | 63.800 | 130.267 | −0.148 | 1.00 | 200.89 | 8 |
| ATOM | 5803 | N | PHE | C | 293 | 65.854 | 129.754 | −0.914 | 1.00 | 97.14 | 7 |
| ATOM | 5804 | CA | PHE | C | 293 | 65.458 | 129.752 | −2.316 | 1.00 | 98.47 | 6 |
| ATOM | 5805 | CB | PHE | C | 293 | 64.614 | 128.511 | −2.634 | 1.00 | 208.87 | 6 |
| ATOM | 5806 | CG | PHE | C | 293 | 64.578 | 128.162 | −4.098 | 1.00 | 208.87 | 6 |
| ATOM | 5807 | CD1 | PHE | C | 293 | 65.752 | 127.851 | −4.779 | 1.00 | 208.87 | 6 |
| ATOM | 5808 | CD2 | PHE | C | 293 | 63.376 | 128.151 | −4.798 | 1.00 | 208.87 | 6 |
| ATOM | 5809 | CE1 | PHE | C | 293 | 65.732 | 127.534 | −6.135 | 1.00 | 208.87 | 6 |
| ATOM | 5810 | CE2 | PHE | C | 293 | 63.344 | 127.835 | −6.156 | 1.00 | 208.87 | 6 |
| ATOM | 5811 | CZ | PHE | C | 293 | 64.526 | 127.527 | −6.824 | 1.00 | 208.87 | 6 |
| ATOM | 5812 | C | PHE | C | 293 | 64.679 | 131.019 | −2.675 | 1.00 | 98.97 | 6 |
| ATOM | 5813 | O | PHE | C | 293 | 64.020 | 131.083 | −3.715 | 1.00 | 99.42 | 8 |
| ATOM | 5814 | N | GLU | C | 294 | 64.748 | 132.020 | −1.802 | 1.00 | 165.72 | 7 |
| ATOM | 5815 | CA | GLU | C | 294 | 64.078 | 133.294 | −2.039 | 1.00 | 166.50 | 6 |
| ATOM | 5816 | CB | GLU | C | 294 | 64.121 | 134.151 | −0.775 | 1.00 | 133.84 | 6 |
| ATOM | 5817 | CG | GLU | C | 294 | 63.130 | 133.742 | 0.283 | 1.00 | 135.17 | 6 |
| ATOM | 5818 | CD | GLU | C | 294 | 61.717 | 134.128 | −0.086 | 1.00 | 136.82 | 6 |
| ATOM | 5819 | OE1 | GLU | C | 294 | 61.464 | 135.338 | −0.270 | 1.00 | 137.55 | 8 |
| ATOM | 5820 | OE2 | GLU | C | 294 | 60.859 | 133.226 | −0.192 | 1.00 | 138.64 | 8 |
| ATOM | 5821 | C | GLU | C | 294 | 64.857 | 133.987 | −3.144 | 1.00 | 166.56 | 6 |
| ATOM | 5822 | O | GLU | C | 294 | 64.294 | 134.476 | −4.126 | 1.00 | 166.41 | 8 |
| ATOM | 5823 | N | ASP | C | 295 | 66.170 | 134.003 | −2.953 | 1.00 | 135.96 | 7 |
| ATOM | 5824 | CA | ASP | C | 295 | 67.117 | 134.607 | −3.874 | 1.00 | 135.93 | 6 |
| ATOM | 5825 | CB | ASP | C | 295 | 66.697 | 136.045 | −4.201 | 1.00 | 86.41 | 6 |
| ATOM | 5826 | CG | ASP | C | 295 | 66.565 | 136.913 | −2.967 | 1.00 | 85.80 | 6 |
| ATOM | 5827 | OD1 | ASP | C | 295 | 65.831 | 136.526 | −2.041 | 1.00 | 84.79 | 8 |
| ATOM | 5828 | OD2 | ASP | C | 295 | 67.200 | 137.985 | −2.927 | 1.00 | 86.23 | 8 |
| ATOM | 5829 | C | ASP | C | 295 | 68.477 | 134.582 | −3.177 | 1.00 | 136.47 | 6 |
| ATOM | 5830 | O | ASP | C | 295 | 68.875 | 135.553 | −2.530 | 1.00 | 136.80 | 8 |
| ATOM | 5831 | N | GLY | C | 296 | 69.173 | 133.455 | −3.293 | 1.00 | 110.37 | 7 |
| ATOM | 5832 | CA | GLY | C | 296 | 70.470 | 133.326 | −2.655 | 1.00 | 110.99 | 6 |
| ATOM | 5833 | C | GLY | C | 296 | 70.378 | 133.109 | −1.157 | 1.00 | 111.59 | 6 |
| ATOM | 5834 | O | GLY | C | 296 | 70.265 | 131.972 | −0.698 | 1.00 | 112.40 | 8 |
| ATOM | 5835 | N | GLU | C | 297 | 70.414 | 134.197 | −0.391 | 1.00 | 170.55 | 7 |
| ATOM | 5836 | CA | GLU | C | 297 | 70.353 | 134.110 | 1.069 | 1.00 | 170.60 | 6 |
| ATOM | 5837 | CB | GLU | C | 297 | 70.454 | 135.504 | 1.701 | 1.00 | 186.63 | 6 |
| ATOM | 5838 | CG | GLU | C | 297 | 69.344 | 136.466 | 1.302 | 1.00 | 188.22 | 6 |
| ATOM | 5839 | CD | GLU | C | 297 | 69.261 | 137.676 | 2.219 | 1.00 | 188.95 | 6 |
| ATOM | 5840 | OE1 | GLU | C | 297 | 70.310 | 138.305 | 2.478 | 1.00 | 189.46 | 8 |
| ATOM | 5841 | OE2 | GLU | C | 297 | 68.143 | 138.004 | 2.673 | 1.00 | 189.15 | 8 |
| ATOM | 5842 | C | GLU | C | 297 | 69.095 | 133.426 | 1.586 | 1.00 | 170.34 | 6 |
| ATOM | 5843 | O | GLU | C | 297 | 68.225 | 133.022 | 0.818 | 1.00 | 170.10 | 8 |
| ATOM | 5844 | N | PHE | C | 298 | 69.019 | 133.309 | 2.906 | 1.00 | 140.37 | 7 |
| ATOM | 5845 | CA | PHE | C | 298 | 67.893 | 132.682 | 3.577 | 1.00 | 140.50 | 6 |
| ATOM | 5846 | CB | PHE | C | 298 | 68.384 | 131.530 | 4.457 | 1.00 | 139.59 | 6 |
| ATOM | 5847 | CG | PHE | C | 298 | 68.795 | 130.305 | 3.697 | 1.00 | 140.10 | 6 |
| ATOM | 5848 | CD1 | PHE | C | 298 | 69.308 | 130.399 | 2.411 | 1.00 | 140.80 | 6 |
| ATOM | 5849 | CD2 | PHE | C | 298 | 68.701 | 129.053 | 4.289 | 1.00 | 139.57 | 6 |
| ATOM | 5850 | CE1 | PHE | C | 298 | 69.714 | 129.262 | 1.721 | 1.00 | 140.82 | 6 |
| ATOM | 5851 | CE2 | PHE | C | 298 | 69.103 | 127.913 | 3.610 | 1.00 | 139.07 | 6 |
| ATOM | 5852 | CZ | PHE | C | 298 | 69.613 | 128.015 | 2.327 | 1.00 | 139.85 | 6 |
| ATOM | 5853 | C | PHE | C | 298 | 67.176 | 133.687 | 4.462 | 1.00 | 140.52 | 6 |
| ATOM | 5854 | O | PHE | C | 298 | 67.815 | 134.447 | 5.185 | 1.00 | 141.28 | 8 |
| ATOM | 5855 | N | LYS | C | 299 | 65.850 | 133.695 | 4.400 | 1.00 | 97.69 | 7 |
| ATOM | 5856 | CA | LYS | C | 299 | 65.072 | 134.590 | 5.240 | 1.00 | 97.41 | 6 |
| ATOM | 5857 | CB | LYS | C | 299 | 64.040 | 135.377 | 4.417 | 1.00 | 101.03 | 6 |
| ATOM | 5858 | CG | LYS | C | 299 | 62.904 | 134.549 | 3.816 | 1.00 | 101.43 | 6 |
| ATOM | 5859 | CD | LYS | C | 299 | 61.770 | 135.448 | 3.323 | 1.00 | 102.35 | 6 |
| ATOM | 5860 | CE | LYS | C | 299 | 60.566 | 134.637 | 2.870 | 1.00 | 103.16 | 6 |
| ATOM | 5861 | NZ | LYS | C | 299 | 59.472 | 135.510 | 2.363 | 1.00 | 103.26 | 7 |
| ATOM | 5862 | C | LYS | C | 299 | 64.374 | 133.736 | 6.296 | 1.00 | 96.80 | 6 |
| ATOM | 5863 | O | LYS | C | 299 | 63.288 | 133.208 | 6.066 | 1.00 | 96.71 | 8 |
| ATOM | 5864 | N | ASP | C | 300 | 65.010 | 133.591 | 7.454 | 1.00 | 125.77 | 7 |
| ATOM | 5865 | CA | ASP | C | 300 | 64.447 | 132.780 | 8.526 | 1.00 | 124.41 | 6 |
| ATOM | 5866 | CB | ASP | C | 300 | 65.450 | 132.619 | 9.667 | 1.00 | 119.73 | 6 |
| ATOM | 5867 | CG | ASP | C | 300 | 65.503 | 133.835 | 10.567 | 1.00 | 120.65 | 6 |
| ATOM | 5868 | OD1 | ASP | C | 300 | 65.894 | 134.916 | 10.083 | 1.00 | 121.00 | 8 |
| ATOM | 5869 | OD2 | ASP | C | 300 | 65.144 | 133.708 | 11.757 | 1.00 | 120.80 | 8 |
| ATOM | 5870 | C | ASP | C | 300 | 63.174 | 133.384 | 9.081 | 1.00 | 123.28 | 6 |
| ATOM | 5871 | O | ASP | C | 300 | 62.922 | 134.580 | 8.948 | 1.00 | 123.66 | 8 |
| ATOM | 5872 | N | GLU | C | 301 | 62.376 | 132.538 | 9.713 | 1.00 | 134.29 | 7 |
| ATOM | 5873 | CA | GLU | C | 301 | 61.129 | 132.969 | 10.312 | 1.00 | 132.48 | 6 |
| ATOM | 5874 | CB | GLU | C | 301 | 59.957 | 132.201 | 9.694 | 1.00 | 85.74 | 6 |
| ATOM | 5875 | CG | GLU | C | 301 | 59.865 | 132.246 | 8.153 | 1.00 | 86.85 | 6 |
| ATOM | 5876 | CD | GLU | C | 301 | 59.436 | 133.597 | 7.591 | 1.00 | 87.33 | 6 |
| ATOM | 5877 | OE1 | GLU | C | 301 | 58.561 | 134.248 | 8.198 | 1.00 | 88.03 | 8 |
| ATOM | 5878 | OE2 | GLU | C | 301 | 59.957 | 133.999 | 6.529 | 1.00 | 87.91 | 8 |
| ATOM | 5879 | C | GLU | C | 301 | 61.224 | 132.687 | 11.816 | 1.00 | 130.86 | 6 |
| ATOM | 5880 | O | GLU | C | 301 | 61.342 | 133.612 | 12.621 | 1.00 | 131.67 | 8 |
| ATOM | 5881 | N | VAL | C | 302 | 61.194 | 131.407 | 12.183 | 1.00 | 150.57 | 7 |
| ATOM | 5882 | CA | VAL | C | 302 | 61.283 | 130.989 | 13.583 | 1.00 | 147.82 | 6 |
| ATOM | 5883 | CB | VAL | C | 302 | 60.219 | 129.905 | 13.907 | 1.00 | 130.83 | 6 |
| ATOM | 5884 | CG1 | VAL | C | 302 | 60.690 | 129.001 | 15.047 | 1.00 | 131.76 | 6 |
| ATOM | 5885 | CG2 | VAL | C | 302 | 58.934 | 130.574 | 14.308 | 1.00 | 131.06 | 6 |
| ATOM | 5886 | C | VAL | C | 302 | 62.662 | 130.445 | 13.945 | 1.00 | 145.09 | 6 |
| ATOM | 5887 | O | VAL | C | 302 | 63.113 | 129.464 | 13.361 | 1.00 | 145.82 | 8 |
| ATOM | 5888 | N | PHE | C | 303 | 63.320 | 131.081 | 14.911 | 1.00 | 103.76 | 7 |
| ATOM | 5889 | CA | PHE | C | 303 | 64.638 | 130.642 | 15.351 | 1.00 | 99.80 | 6 |
| ATOM | 5890 | CB | PHE | C | 303 | 65.582 | 130.499 | 14.166 | 1.00 | 43.70 | 6 |
| ATOM | 5891 | CG | PHE | C | 303 | 65.991 | 129.084 | 13.898 | 1.00 | 41.90 | 6 |
| ATOM | 5892 | CD1 | PHE | C | 303 | 65.617 | 128.442 | 12.728 | 1.00 | 41.03 | 6 |
| ATOM | 5893 | CD2 | PHE | C | 303 | 66.751 | 128.386 | 14.819 | 1.00 | 41.62 | 6 |
| ATOM | 5894 | CE1 | PHE | C | 303 | 65.992 | 127.125 | 12.478 | 1.00 | 40.27 | 6 |
| ATOM | 5895 | CE2 | PHE | C | 303 | 67.131 | 127.066 | 14.574 | 1.00 | 42.22 | 6 |
| ATOM | 5896 | CZ | PHE | C | 303 | 66.749 | 126.437 | 13.399 | 1.00 | 41.12 | 6 |
| ATOM | 5897 | C | PHE | C | 303 | 65.257 | 131.580 | 16.377 | 1.00 | 98.71 | 6 |
| ATOM | 5898 | O | PHE | C | 303 | 65.676 | 131.146 | 17.452 | 1.00 | 99.30 | 8 |
| ATOM | 5899 | N | LEU | C | 304 | 65.342 | 132.861 | 16.034 | 1.00 | 121.07 | 7 |
| ATOM | 5900 | CA | LEU | C | 304 | 65.881 | 133.866 | 16.947 | 1.00 | 117.89 | 6 |
| ATOM | 5901 | CB | LEU | C | 304 | 66.549 | 135.010 | 16.172 | 1.00 | 58.33 | 6 |
| ATOM | 5902 | CG | LEU | C | 304 | 66.257 | 136.475 | 16.535 | 1.00 | 57.60 | 6 |
| ATOM | 5903 | CD1 | LEU | C | 304 | 67.456 | 137.347 | 16.186 | 1.00 | 57.31 | 6 |
| ATOM | 5904 | CD2 | LEU | C | 304 | 65.012 | 136.958 | 15.801 | 1.00 | 57.48 | 6 |
| ATOM | 5905 | C | LEU | C | 304 | 64.746 | 134.406 | 17.801 | 1.00 | 116.09 | 6 |
| ATOM | 5906 | O | LEU | C | 304 | 64.939 | 134.703 | 18.976 | 1.00 | 116.63 | 8 |
| ATOM | 5907 | N | PRO | C | 305 | 63.542 | 134.543 | 17.215 | 1.00 | 79.40 | 7 |
| ATOM | 5908 | CD | PRO | C | 305 | 63.180 | 134.343 | 15.802 | 1.00 | 52.04 | 6 |
| ATOM | 5909 | CA | PRO | C | 305 | 62.390 | 135.052 | 17.962 | 1.00 | 77.80 | 6 |
| ATOM | 5910 | CB | PRO | C | 305 | 61.261 | 134.996 | 16.934 | 1.00 | 51.78 | 6 |
| ATOM | 5911 | CG | PRO | C | 305 | 61.974 | 135.230 | 15.662 | 1.00 | 52.39 | 6 |
| ATOM | 5912 | C | PRO | C | 305 | 62.115 | 134.171 | 19.175 | 1.00 | 76.42 | 6 |
| ATOM | 5913 | O | PRO | C | 305 | 61.688 | 134.652 | 20.230 | 1.00 | 75.90 | 8 |
| ATOM | 5914 | N | THR | C | 306 | 62.353 | 132.875 | 19.013 | 1.00 | 64.70 | 7 |
| ATOM | 5915 | CA | THR | C | 306 | 62.153 | 131.944 | 20.103 | 1.00 | 62.16 | 6 |
| ATOM | 5916 | CB | THR | C | 306 | 62.114 | 130.496 | 19.588 | 1.00 | 37.62 | 6 |
| ATOM | 5917 | OG1 | THR | C | 306 | 60.961 | 129.845 | 20.128 | 1.00 | 37.49 | 8 |
| ATOM | 5918 | CG2 | THR | C | 306 | 63.355 | 129.717 | 20.003 | 1.00 | 36.49 | 6 |
| ATOM | 5919 | C | THR | C | 306 | 63.321 | 132.149 | 21.062 | 1.00 | 61.79 | 6 |
| ATOM | 5920 | O | THR | C | 306 | 63.137 | 132.197 | 22.279 | 1.00 | 62.74 | 8 |
| ATOM | 5921 | N | LEU | C | 307 | 64.520 | 132.283 | 20.498 | 1.00 | 68.15 | 7 |
| ATOM | 5922 | CA | LEU | C | 307 | 65.735 | 132.514 | 21.276 | 1.00 | 66.39 | 6 |
| ATOM | 5923 | CB | LEU | C | 307 | 66.900 | 132.800 | 20.322 | 1.00 | 49.86 | 6 |
| ATOM | 5924 | CG | LEU | C | 307 | 68.341 | 132.691 | 20.827 | 1.00 | 48.12 | 6 |
| ATOM | 5925 | CD1 | LEU | C | 307 | 68.652 | 131.276 | 21.309 | 1.00 | 45.23 | 6 |
| ATOM | 5926 | CD2 | LEU | C | 307 | 69.268 | 133.098 | 19.693 | 1.00 | 47.43 | 6 |
| ATOM | 5927 | C | LEU | C | 307 | 65.405 | 133.739 | 22.121 | 1.00 | 66.43 | 6 |
| ATOM | 5928 | O | LEU | C | 307 | 65.789 | 133.850 | 23.285 | 1.00 | 66.27 | 8 |
| ATOM | 5929 | N | ARG | C | 308 | 64.663 | 134.647 | 21.499 | 1.00 | 50.53 | 7 |
| ATOM | 5930 | CA | ARG | C | 308 | 64.186 | 135.871 | 22.129 | 1.00 | 49.79 | 6 |
| ATOM | 5931 | CB | ARG | C | 308 | 63.410 | 136.697 | 21.090 | 1.00 | 71.35 | 6 |
| ATOM | 5932 | CG | ARG | C | 308 | 62.901 | 138.065 | 21.532 | 1.00 | 73.99 | 6 |
| ATOM | 5933 | CD | ARG | C | 308 | 62.560 | 138.925 | 20.304 | 1.00 | 75.25 | 6 |
| ATOM | 5934 | NE | ARG | C | 308 | 61.647 | 138.249 | 19.382 | 1.00 | 75.78 | 7 |
| ATOM | 5935 | CZ | ARG | C | 308 | 61.333 | 138.696 | 18.169 | 1.00 | 75.70 | 6 |
| ATOM | 5936 | NH1 | ARG | C | 308 | 61.855 | 139.825 | 17.713 | 1.00 | 75.04 | 7 |
| ATOM | 5937 | NH2 | ARG | C | 308 | 60.493 | 138.011 | 17.408 | 1.00 | 76.65 | 7 |
| ATOM | 5938 | C | ARG | C | 308 | 63.260 | 135.391 | 23.240 | 1.00 | 48.91 | 6 |
| ATOM | 5939 | O | ARG | C | 308 | 63.627 | 135.416 | 24.420 | 1.00 | 48.85 | 8 |
| ATOM | 5940 | N | TYR | C | 309 | 62.074 | 134.923 | 22.841 | 1.00 | 59.73 | 7 |
| ATOM | 5941 | CA | TYR | C | 309 | 61.063 | 134.401 | 23.767 | 1.00 | 57.75 | 6 |
| ATOM | 5942 | CB | TYR | C | 309 | 59.986 | 133.625 | 22.997 | 1.00 | 46.74 | 6 |
| ATOM | 5943 | CG | TYR | C | 309 | 58.894 | 132.982 | 23.847 | 1.00 | 44.05 | 6 |
| ATOM | 5944 | CD1 | TYR | C | 309 | 57.800 | 133.719 | 24.291 | 1.00 | 44.13 | 6 |
| ATOM | 5945 | CE1 | TYR | C | 309 | 56.776 | 133.114 | 25.015 | 1.00 | 44.22 | 6 |
| ATOM | 5946 | CD2 | TYR | C | 309 | 58.938 | 131.622 | 24.161 | 1.00 | 42.45 | 6 |
| ATOM | 5947 | CE2 | TYR | C | 309 | 57.932 | 131.011 | 24.880 | 1.00 | 42.72 | 6 |
| ATOM | 5948 | CZ | TYR | C | 309 | 56.849 | 131.759 | 25.301 | 1.00 | 43.96 | 6 |
| ATOM | 5949 | OH | TYR | C | 309 | 55.813 | 131.147 | 25.968 | 1.00 | 44.56 | 8 |
| ATOM | 5950 | C | TYR | C | 309 | 61.698 | 133.487 | 24.805 | 1.00 | 56.45 | 6 |
| ATOM | 5951 | O | TYR | C | 309 | 61.065 | 133.123 | 25.785 | 1.00 | 55.94 | 8 |
| ATOM | 5952 | N | LEU | C | 310 | 62.943 | 133.095 | 24.585 | 1.00 | 18.61 | 7 |
| ATOM | 5953 | CA | LEU | C | 310 | 63.594 | 132.257 | 25.554 | 1.00 | 19.45 | 6 |
| ATOM | 5954 | CB | LEU | C | 310 | 64.806 | 131.545 | 24.969 | 1.00 | 22.17 | 6 |
| ATOM | 5955 | CG | LEU | C | 310 | 65.500 | 130.689 | 26.043 | 1.00 | 20.12 | 6 |
| ATOM | 5956 | CD1 | LEU | C | 310 | 64.601 | 129.509 | 26.355 | 1.00 | 17.82 | 6 |
| ATOM | 5957 | CD2 | LEU | C | 310 | 66.891 | 130.218 | 25.601 | 1.00 | 18.38 | 6 |
| ATOM | 5958 | C | LEU | C | 310 | 64.056 | 133.133 | 26.699 | 1.00 | 21.90 | 6 |
| ATOM | 5959 | O | LEU | C | 310 | 63.604 | 132.979 | 27.837 | 1.00 | 22.91 | 8 |
| ATOM | 5960 | N | PHE | C | 311 | 64.966 | 134.057 | 26.404 | 1.00 | 63.18 | 7 |
| ATOM | 5961 | CA | PHE | C | 311 | 65.502 | 134.952 | 27.430 | 1.00 | 64.52 | 6 |
| ATOM | 5962 | CB | PHE | C | 311 | 66.446 | 135.981 | 26.784 | 1.00 | 56.38 | 6 |
| ATOM | 5963 | CG | PHE | C | 311 | 67.695 | 135.373 | 26.181 | 1.00 | 56.52 | 6 |
| ATOM | 5964 | CD1 | PHE | C | 311 | 68.669 | 136.180 | 25.608 | 1.00 | 56.80 | 6 |
| ATOM | 5965 | CD2 | PHE | C | 311 | 67.897 | 133.998 | 26.186 | 1.00 | 56.67 | 6 |
| ATOM | 5966 | CE1 | PHE | C | 311 | 69.825 | 135.631 | 25.049 | 1.00 | 56.17 | 6 |
| ATOM | 5967 | CE2 | PHE | C | 311 | 69.046 | 133.446 | 25.630 | 1.00 | 56.99 | 6 |
| ATOM | 5968 | CZ | PHE | C | 311 | 70.011 | 134.268 | 25.060 | 1.00 | 56.37 | 6 |
| ATOM | 5969 | C | PHE | C | 311 | 64.378 | 135.643 | 28.208 | 1.00 | 65.71 | 6 |
| ATOM | 5970 | O | PHE | C | 311 | 64.494 | 135.889 | 29.408 | 1.00 | 65.06 | 8 |
| ATOM | 5971 | N | ALA | C | 312 | 63.287 | 135.938 | 27.509 | 1.00 | 109.29 | 7 |
| ATOM | 5972 | CA | ALA | C | 312 | 62.127 | 136.570 | 28.119 | 1.00 | 110.75 | 6 |
| ATOM | 5973 | CB | ALA | C | 312 | 61.064 | 136.820 | 27.067 | 1.00 | 51.75 | 6 |
| ATOM | 5974 | C | ALA | C | 312 | 61.599 | 135.606 | 29.167 | 1.00 | 111.67 | 6 |
| ATOM | 5975 | O | ALA | C | 312 | 60.890 | 135.989 | 30.093 | 1.00 | 112.90 | 8 |
| ATOM | 5976 | N | LEU | C | 313 | 61.958 | 134.342 | 28.993 | 1.00 | 56.14 | 7 |
| ATOM | 5977 | CA | LEU | C | 313 | 61.558 | 133.269 | 29.883 | 1.00 | 55.56 | 6 |
| ATOM | 5978 | CB | LEU | C | 313 | 61.211 | 132.031 | 29.054 | 1.00 | 15.69 | 6 |
| ATOM | 5979 | CG | LEU | C | 313 | 61.013 | 130.634 | 29.656 | 1.00 | 14.79 | 6 |
| ATOM | 5980 | CD1 | LEU | C | 313 | 60.006 | 130.649 | 30.788 | 1.00 | 15.15 | 6 |
| ATOM | 5981 | CD2 | LEU | C | 313 | 60.543 | 129.704 | 28.560 | 1.00 | 13.87 | 6 |
| ATOM | 5982 | C | LEU | C | 313 | 62.729 | 132.989 | 30.800 | 1.00 | 56.68 | 6 |
| ATOM | 5983 | O | LEU | C | 313 | 62.718 | 132.033 | 31.571 | 1.00 | 57.42 | 8 |
| ATOM | 5984 | N | THR | C | 314 | 63.750 | 133.833 | 30.717 | 1.00 | 59.39 | 7 |
| ATOM | 5985 | CA | THR | C | 314 | 64.920 | 133.656 | 31.559 | 1.00 | 61.60 | 6 |
| ATOM | 5986 | CB | THR | C | 314 | 66.096 | 133.099 | 30.733 | 1.00 | 48.58 | 6 |
| ATOM | 5987 | OG1 | THR | C | 314 | 65.637 | 132.004 | 29.927 | 1.00 | 47.77 | 8 |
| ATOM | 5988 | CG2 | THR | C | 314 | 67.196 | 132.590 | 31.656 | 1.00 | 48.80 | 6 |
| ATOM | 5989 | C | THR | C | 314 | 65.315 | 134.972 | 32.246 | 1.00 | 63.97 | 6 |
| ATOM | 5990 | O | THR | C | 314 | 66.455 | 135.439 | 32.132 | 1.00 | 63.96 | 8 |
| ATOM | 5991 | N | ALA | C | 315 | 64.339 | 135.543 | 32.958 | 1.00 | 73.83 | 7 |
| ATOM | 5992 | CA | ALA | C | 315 | 64.444 | 136.793 | 33.722 | 1.00 | 74.62 | 6 |
| ATOM | 5993 | CB | ALA | C | 315 | 65.837 | 137.419 | 33.583 | 1.00 | 65.98 | 6 |
| ATOM | 5994 | C | ALA | C | 315 | 63.372 | 137.792 | 33.284 | 1.00 | 75.31 | 6 |
| ATOM | 5995 | O | ALA | C | 315 | 63.142 | 138.798 | 33.951 | 1.00 | 75.44 | 8 |
| ATOM | 5996 | N | GLY | C | 316 | 62.704 | 137.502 | 32.171 | 1.00 | 61.65 | 7 |
| ATOM | 5997 | CA | GLY | C | 316 | 61.675 | 138.396 | 31.672 | 1.00 | 63.94 | 6 |
| ATOM | 5998 | C | GLY | C | 316 | 60.316 | 138.208 | 32.318 | 1.00 | 65.80 | 6 |
| ATOM | 5999 | O | GLY | C | 316 | 60.215 | 137.699 | 33.440 | 1.00 | 65.97 | 8 |
| ATOM | 6000 | N | VAL | C | 317 | 59.270 | 138.625 | 31.604 | 1.00 | 71.99 | 7 |
| ATOM | 6001 | CA | VAL | C | 317 | 57.897 | 138.515 | 32.094 | 1.00 | 73.19 | 6 |
| ATOM | 6002 | CB | VAL | C | 317 | 57.187 | 139.884 | 32.089 | 1.00 | 50.58 | 6 |
| ATOM | 6003 | CG1 | VAL | C | 317 | 55.684 | 139.701 | 32.287 | 1.00 | 50.11 | 6 |
| ATOM | 6004 | CG2 | VAL | C | 317 | 57.753 | 140.758 | 33.199 | 1.00 | 50.53 | 6 |
| ATOM | 6005 | C | VAL | C | 317 | 57.003 | 137.513 | 31.356 | 1.00 | 75.00 | 6 |
| ATOM | 6006 | O | VAL | C | 317 | 56.214 | 136.816 | 31.988 | 1.00 | 75.45 | 8 |
| ATOM | 6007 | N | PRO | C | 318 | 57.107 | 137.421 | 30.016 | 1.00 | 149.53 | 7 |
| ATOM | 6008 | CD | PRO | C | 318 | 58.077 | 138.026 | 29.086 | 1.00 | 68.35 | 6 |
| ATOM | 6009 | CA | PRO | C | 318 | 56.246 | 136.457 | 29.315 | 1.00 | 150.81 | 6 |
| ATOM | 6010 | CB | PRO | C | 318 | 56.628 | 136.657 | 27.849 | 1.00 | 68.99 | 6 |
| ATOM | 6011 | CG | PRO | C | 318 | 58.067 | 137.042 | 27.932 | 1.00 | 68.35 | 6 |
| ATOM | 6012 | C | PRO | C | 318 | 56.483 | 135.027 | 29.801 | 1.00 | 152.05 | 6 |
| ATOM | 6013 | O | PRO | C | 318 | 55.666 | 134.136 | 29.583 | 1.00 | 151.58 | 8 |
| ATOM | 6014 | N | GLY | C | 319 | 57.620 | 134.840 | 30.465 | 1.00 | 73.22 | 7 |
| ATOM | 6015 | CA | GLY | C | 319 | 58.015 | 133.559 | 31.023 | 1.00 | 74.96 | 6 |
| ATOM | 6016 | C | GLY | C | 319 | 57.383 | 132.312 | 30.452 | 1.00 | 76.68 | 6 |
| ATOM | 6017 | O | GLY | C | 319 | 57.790 | 131.826 | 29.400 | 1.00 | 76.07 | 8 |
| ATOM | 6018 | N | HIS | C | 320 | 56.382 | 131.794 | 31.152 | 1.00 | 84.00 | 7 |
| ATOM | 6019 | CA | HIS | C | 320 | 55.699 | 130.575 | 30.734 | 1.00 | 86.17 | 6 |
| ATOM | 6020 | CB | HIS | C | 320 | 54.877 | 130.812 | 29.460 | 1.00 | 158.67 | 6 |
| ATOM | 6021 | CG | HIS | C | 320 | 53.751 | 131.785 | 29.638 | 1.00 | 159.50 | 6 |
| ATOM | 6022 | CD2 | HIS | C | 320 | 53.332 | 132.816 | 28.866 | 1.00 | 159.65 | 6 |
| ATOM | 6023 | ND1 | HIS | C | 320 | 52.900 | 131.749 | 30.722 | 1.00 | 159.24 | 7 |
| ATOM | 6024 | CE1 | HIS | C | 320 | 52.008 | 132.716 | 30.611 | 1.00 | 159.88 | 6 |
| ATOM | 6025 | NE2 | HIS | C | 320 | 52.248 | 133.379 | 29.494 | 1.00 | 159.94 | 7 |
| ATOM | 6026 | C | HIS | C | 320 | 56.719 | 129.458 | 30.510 | 1.00 | 86.95 | 6 |
| ATOM | 6027 | O | HIS | C | 320 | 57.403 | 129.405 | 29.488 | 1.00 | 86.53 | 8 |
| ATOM | 6028 | N | GLU | C | 321 | 56.829 | 128.577 | 31.495 | 1.00 | 135.10 | 7 |
| ATOM | 6029 | CA | GLU | C | 321 | 57.757 | 127.466 | 31.414 | 1.00 | 136.56 | 6 |
| ATOM | 6030 | CB | GLU | C | 321 | 58.179 | 127.028 | 32.817 | 1.00 | 165.69 | 6 |
| ATOM | 6031 | CG | GLU | C | 321 | 58.904 | 128.108 | 33.613 | 1.00 | 167.45 | 6 |
| ATOM | 6032 | CD | GLU | C | 321 | 58.115 | 128.592 | 34.822 | 1.00 | 168.27 | 6 |
| ATOM | 6033 | OE1 | GLU | C | 321 | 56.993 | 129.110 | 34.642 | 1.00 | 169.04 | 8 |
| ATOM | 6034 | OE2 | GLU | C | 321 | 58.621 | 128.457 | 35.956 | 1.00 | 167.77 | 8 |
| ATOM | 6035 | C | GLU | C | 321 | 57.072 | 126.320 | 30.692 | 1.00 | 137.60 | 6 |
| ATOM | 6036 | O | GLU | C | 321 | 57.513 | 125.177 | 30.783 | 1.00 | 138.15 | 8 |
| ATOM | 6037 | N | VAL | C | 322 | 55.993 | 126.635 | 29.977 | 1.00 | 99.38 | 7 |
| ATOM | 6038 | CA | VAL | C | 322 | 55.236 | 125.628 | 29.233 | 1.00 | 100.56 | 6 |
| ATOM | 6039 | CB | VAL | C | 322 | 54.463 | 126.252 | 28.026 | 1.00 | 100.02 | 6 |
| ATOM | 6040 | CG1 | VAL | C | 322 | 55.386 | 127.099 | 27.186 | 1.00 | 100.63 | 6 |
| ATOM | 6041 | CG2 | VAL | C | 322 | 53.849 | 125.155 | 27.168 | 1.00 | 99.26 | 6 |
| ATOM | 6042 | C | VAL | C | 322 | 56.139 | 124.512 | 28.726 | 1.00 | 101.41 | 6 |
| ATOM | 6043 | O | VAL | C | 322 | 57.015 | 124.730 | 27.890 | 1.00 | 101.84 | 8 |
| ATOM | 6044 | N | ALA | C | 323 | 55.922 | 123.317 | 29.260 | 1.00 | 115.83 | 7 |
| ATOM | 6045 | CA | ALA | C | 323 | 56.704 | 122.151 | 28.881 | 1.00 | 116.94 | 6 |
| ATOM | 6046 | CB | ALA | C | 323 | 57.288 | 121.498 | 30.132 | 1.00 | 101.48 | 6 |
| ATOM | 6047 | C | ALA | C | 323 | 55.798 | 121.169 | 28.143 | 1.00 | 117.29 | 6 |
| ATOM | 6048 | O | ALA | C | 323 | 54.783 | 121.571 | 27.572 | 1.00 | 117.57 | 8 |
| ATOM | 6049 | N | ASP | C | 324 | 56.164 | 119.890 | 28.133 | 1.00 | 90.38 | 7 |
| ATOM | 6050 | CA | ASP | C | 324 | 55.321 | 118.898 | 27.498 | 1.00 | 90.03 | 6 |
| ATOM | 6051 | CB | ASP | C | 324 | 56.020 | 117.535 | 27.468 | 1.00 | 208.87 | 6 |
| ATOM | 6052 | CG | ASP | C | 324 | 56.492 | 117.085 | 28.837 | 1.00 | 208.87 | 6 |
| ATOM | 6053 | OD1 | ASP | C | 324 | 57.291 | 117.813 | 29.465 | 1.00 | 208.87 | 8 |
| ATOM | 6054 | OD2 | ASP | C | 324 | 56.065 | 115.997 | 29.279 | 1.00 | 208.87 | 8 |
| ATOM | 6055 | C | ASP | C | 324 | 54.108 | 118.875 | 28.414 | 1.00 | 88.37 | 6 |
| ATOM | 6056 | O | ASP | C | 324 | 53.586 | 117.822 | 28.765 | 1.00 | 87.10 | 8 |
| ATOM | 6057 | N | ALA | C | 325 | 53.698 | 120.081 | 28.799 | 1.00 | 129.49 | 7 |
| ATOM | 6058 | CA | ALA | C | 325 | 52.588 | 120.367 | 29.689 | 1.00 | 128.97 | 6 |
| ATOM | 6059 | CB | ALA | C | 325 | 52.037 | 121.746 | 29.362 | 1.00 | 37.90 | 6 |
| ATOM | 6060 | C | ALA | C | 325 | 51.474 | 119.329 | 29.703 | 1.00 | 128.58 | 6 |
| ATOM | 6061 | O | ALA | C | 325 | 50.558 | 119.403 | 30.520 | 1.00 | 129.16 | 8 |
| ATOM | 6062 | N | ASP | C | 326 | 51.550 | 118.364 | 28.798 | 1.00 | 84.98 | 7 |
| ATOM | 6063 | CA | ASP | C | 326 | 50.574 | 117.296 | 28.732 | 1.00 | 83.82 | 6 |
| ATOM | 6064 | CB | ASP | C | 326 | 50.641 | 116.453 | 30.004 | 1.00 | 99.04 | 6 |
| ATOM | 6065 | CG | ASP | C | 326 | 52.050 | 115.992 | 30.321 | 1.00 | 99.14 | 6 |
| ATOM | 6066 | OD1 | ASP | C | 326 | 52.692 | 115.374 | 29.444 | 1.00 | 98.26 | 8 |
| ATOM | 6067 | OD2 | ASP | C | 326 | 52.516 | 116.244 | 31.451 | 1.00 | 99.68 | 8 |
| ATOM | 6068 | C | ASP | C | 326 | 49.179 | 117.850 | 28.542 | 1.00 | 82.28 | 6 |
| ATOM | 6069 | O | ASP | C | 326 | 48.217 | 117.099 | 28.405 | 1.00 | 83.20 | 8 |
| ATOM | 6070 | N | HIS | C | 327 | 49.065 | 119.170 | 28.552 | 1.00 | 131.67 | 7 |
| ATOM | 6071 | CA | HIS | C | 327 | 47.772 | 119.780 | 28.340 | 1.00 | 129.72 | 6 |
| ATOM | 6072 | CB | HIS | C | 327 | 47.902 | 121.302 | 28.263 | 1.00 | 67.84 | 6 |
| ATOM | 6073 | CG | HIS | C | 327 | 46.595 | 122.014 | 28.079 | 1.00 | 66.29 | 6 |
| ATOM | 6074 | CD2 | HIS | C | 327 | 46.013 | 122.538 | 26.974 | 1.00 | 66.15 | 6 |
| ATOM | 6075 | ND1 | HIS | C | 327 | 45.706 | 122.222 | 29.111 | 1.00 | 65.92 | 7 |
| ATOM | 6076 | CE1 | HIS | C | 327 | 44.633 | 122.839 | 28.651 | 1.00 | 66.32 | 6 |
| ATOM | 6077 | NE2 | HIS | C | 327 | 44.794 | 123.043 | 27.357 | 1.00 | 66.58 | 7 |
| ATOM | 6078 | C | HIS | C | 327 | 47.406 | 119.229 | 26.978 | 1.00 | 127.76 | 6 |
| ATOM | 6079 | O | HIS | C | 327 | 48.203 | 119.330 | 26.050 | 1.00 | 127.72 | 8 |
| ATOM | 6080 | N | LEU | C | 328 | 46.235 | 118.614 | 26.850 | 1.00 | 89.73 | 7 |
| ATOM | 6081 | CA | LEU | C | 328 | 45.844 | 118.086 | 25.553 | 1.00 | 85.71 | 6 |
| ATOM | 6082 | CB | LEU | C | 328 | 44.593 | 117.232 | 25.656 | 1.00 | 33.75 | 6 |
| ATOM | 6083 | CG | LEU | C | 328 | 44.896 | 115.768 | 25.924 | 1.00 | 32.84 | 6 |
| ATOM | 6084 | CD1 | LEU | C | 328 | 45.556 | 115.634 | 27.290 | 1.00 | 30.73 | 6 |
| ATOM | 6085 | CD2 | LEU | C | 328 | 43.613 | 114.956 | 25.846 | 1.00 | 34.53 | 6 |
| ATOM | 6086 | C | LEU | C | 328 | 45.628 | 119.200 | 24.547 | 1.00 | 83.13 | 6 |
| ATOM | 6087 | O | LEU | C | 328 | 44.682 | 119.185 | 23.766 | 1.00 | 83.74 | 8 |
| ATOM | 6088 | N | GLY | C | 329 | 46.511 | 120.185 | 24.611 | 1.00 | 95.19 | 7 |
| ATOM | 6089 | CA | GLY | C | 329 | 46.493 | 121.295 | 23.690 | 1.00 | 91.47 | 6 |
| ATOM | 6090 | C | GLY | C | 329 | 47.828 | 121.088 | 23.015 | 1.00 | 88.66 | 6 |
| ATOM | 6091 | O | GLY | C | 329 | 48.204 | 121.800 | 22.087 | 1.00 | 90.13 | 8 |
| ATOM | 6092 | N | ASN | C | 330 | 48.546 | 120.081 | 23.511 | 1.00 | 70.19 | 7 |
| ATOM | 6093 | CA | ASN | C | 330 | 49.861 | 119.709 | 22.999 | 1.00 | 65.88 | 6 |
| ATOM | 6094 | CB | ASN | C | 330 | 50.958 | 120.221 | 23.938 | 1.00 | 42.25 | 6 |
| ATOM | 6095 | CG | ASN | C | 330 | 50.787 | 121.686 | 24.286 | 1.00 | 39.89 | 6 |
| ATOM | 6096 | OD1 | ASN | C | 330 | 50.329 | 122.468 | 23.460 | 1.00 | 37.44 | 8 |
| ATOM | 6097 | ND2 | ASN | C | 330 | 51.167 | 122.065 | 25.508 | 1.00 | 37.85 | 7 |
| ATOM | 6098 | C | ASN | C | 330 | 49.995 | 118.190 | 22.841 | 1.00 | 63.95 | 6 |
| ATOM | 6099 | O | ASN | C | 330 | 51.087 | 117.680 | 22.605 | 1.00 | 65.25 | 8 |
| ATOM | 6100 | N | ARG | C | 331 | 48.879 | 117.476 | 22.977 | 1.00 | 31.86 | 7 |
| ATOM | 6101 | CA | ARG | C | 331 | 48.865 | 116.024 | 22.846 | 1.00 | 28.53 | 6 |
| ATOM | 6102 | CB | ARG | C | 331 | 48.153 | 115.381 | 24.030 | 1.00 | 42.69 | 6 |
| ATOM | 6103 | CG | ARG | C | 331 | 49.024 | 115.241 | 25.250 | 1.00 | 42.49 | 6 |
| ATOM | 6104 | CD | ARG | C | 331 | 49.702 | 113.896 | 25.297 | 1.00 | 41.46 | 6 |
| ATOM | 6105 | NE | ARG | C | 331 | 50.969 | 113.996 | 26.005 | 1.00 | 41.16 | 7 |
| ATOM | 6106 | CZ | ARG | C | 331 | 51.525 | 113.016 | 26.714 | 1.00 | 41.94 | 6 |
| ATOM | 6107 | NH1 | ARG | C | 331 | 50.928 | 111.829 | 26.831 | 1.00 | 41.98 | 7 |
| ATOM | 6108 | NH2 | ARG | C | 331 | 52.689 | 113.234 | 27.317 | 1.00 | 42.25 | 7 |
| ATOM | 6109 | C | ARG | C | 331 | 48.168 | 115.619 | 21.579 | 1.00 | 27.55 | 6 |
| ATOM | 6110 | O | ARG | C | 331 | 46.963 | 115.441 | 21.547 | 1.00 | 26.44 | 8 |
| ATOM | 6111 | N | ARG | C | 332 | 48.952 | 115.471 | 20.530 | 1.00 | 63.72 | 7 |
| ATOM | 6112 | CA | ARG | C | 332 | 48.440 | 115.079 | 19.235 | 1.00 | 63.94 | 6 |
| ATOM | 6113 | CB | ARG | C | 332 | 49.574 | 115.223 | 18.210 | 1.00 | 73.34 | 6 |
| ATOM | 6114 | CG | ARG | C | 332 | 49.251 | 114.856 | 16.779 | 1.00 | 75.34 | 6 |
| ATOM | 6115 | CD | ARG | C | 332 | 48.450 | 115.921 | 16.106 | 1.00 | 78.35 | 6 |
| ATOM | 6116 | NE | ARG | C | 332 | 48.485 | 115.736 | 14.666 | 1.00 | 83.95 | 7 |
| ATOM | 6117 | CZ | ARG | C | 332 | 47.961 | 116.591 | 13.796 | 1.00 | 87.69 | 6 |
| ATOM | 6118 | NH1 | ARG | C | 332 | 47.355 | 117.694 | 14.222 | 1.00 | 88.64 | 7 |
| ATOM | 6119 | NH2 | ARG | C | 332 | 48.058 | 116.352 | 12.494 | 1.00 | 90.07 | 7 |
| ATOM | 6120 | C | ARG | C | 332 | 47.948 | 113.628 | 19.296 | 1.00 | 62.87 | 6 |
| ATOM | 6121 | O | ARG | C | 332 | 48.369 | 112.851 | 20.157 | 1.00 | 61.84 | 8 |
| ATOM | 6122 | N | ILE | C | 333 | 47.040 | 113.279 | 18.386 | 1.00 | 62.90 | 7 |
| ATOM | 6123 | CA | ILE | C | 333 | 46.515 | 111.922 | 18.289 | 1.00 | 60.22 | 6 |
| ATOM | 6124 | CB | ILE | C | 333 | 45.088 | 111.824 | 18.812 | 1.00 | 18.02 | 6 |
| ATOM | 6125 | CG2 | ILE | C | 333 | 44.509 | 110.467 | 18.476 | 1.00 | 16.60 | 6 |
| ATOM | 6126 | CG1 | ILE | C | 333 | 45.096 | 112.057 | 20.326 | 1.00 | 18.58 | 6 |
| ATOM | 6127 | CD1 | ILE | C | 333 | 43.788 | 111.693 | 21.050 | 1.00 | 20.84 | 6 |
| ATOM | 6128 | C | ILE | C | 333 | 46.541 | 111.423 | 16.848 | 1.00 | 60.00 | 6 |
| ATOM | 6129 | O | ILE | C | 333 | 45.592 | 111.616 | 16.089 | 1.00 | 60.88 | 8 |
| ATOM | 6130 | N | ARG | C | 334 | 47.645 | 110.788 | 16.476 | 1.00 | 40.82 | 7 |
| ATOM | 6131 | CA | ARG | C | 334 | 47.804 | 110.249 | 15.140 | 1.00 | 39.79 | 6 |
| ATOM | 6132 | CB | ARG | C | 334 | 49.236 | 109.762 | 14.940 | 1.00 | 48.95 | 6 |
| ATOM | 6133 | CG | ARG | C | 334 | 50.248 | 110.869 | 14.856 | 1.00 | 50.53 | 6 |
| ATOM | 6134 | CD | ARG | C | 334 | 51.576 | 110.327 | 14.389 | 1.00 | 53.11 | 6 |
| ATOM | 6135 | NE | ARG | C | 334 | 52.108 | 109.330 | 15.310 | 1.00 | 55.57 | 7 |
| ATOM | 6136 | CZ | ARG | C | 334 | 53.243 | 108.669 | 15.104 | 1.00 | 58.22 | 6 |
| ATOM | 6137 | NH1 | ARG | C | 334 | 53.954 | 108.905 | 14.007 | 1.00 | 59.53 | 7 |
| ATOM | 6138 | NH2 | ARG | C | 334 | 53.676 | 107.776 | 15.986 | 1.00 | 59.09 | 7 |
| ATOM | 6139 | C | ARG | C | 334 | 46.833 | 109.104 | 14.857 | 1.00 | 38.72 | 6 |
| ATOM | 6140 | O | ARG | C | 334 | 47.088 | 107.950 | 15.206 | 1.00 | 38.77 | 8 |
| ATOM | 6141 | N | THR | C | 335 | 45.719 | 109.436 | 14.220 | 1.00 | 27.72 | 7 |
| ATOM | 6142 | CA | THR | C | 335 | 44.711 | 108.451 | 13.858 | 1.00 | 26.86 | 6 |
| ATOM | 6143 | CB | THR | C | 335 | 43.524 | 109.147 | 13.142 | 1.00 | 52.92 | 6 |
| ATOM | 6144 | OG1 | THR | C | 335 | 43.737 | 110.568 | 13.137 | 1.00 | 52.44 | 8 |
| ATOM | 6145 | CG2 | THR | C | 335 | 42.214 | 108.858 | 13.862 | 1.00 | 53.20 | 6 |
| ATOM | 6146 | C | THR | C | 335 | 45.374 | 107.445 | 12.909 | 1.00 | 26.26 | 6 |
| ATOM | 6147 | O | THR | C | 335 | 46.487 | 107.680 | 12.439 | 1.00 | 26.38 | 8 |
| ATOM | 6148 | N | VAL | C | 336 | 44.708 | 106.328 | 12.625 | 1.00 | 19.30 | 7 |
| ATOM | 6149 | CA | VAL | C | 336 | 45.295 | 105.333 | 11.724 | 1.00 | 19.05 | 6 |
| ATOM | 6150 | CB | VAL | C | 336 | 44.296 | 104.234 | 11.295 | 1.00 | 19.08 | 6 |
| ATOM | 6151 | CG1 | VAL | C | 336 | 45.051 | 103.070 | 10.675 | 1.00 | 17.32 | 6 |
| ATOM | 6152 | CG2 | VAL | C | 336 | 43.466 | 103.781 | 12.471 | 1.00 | 21.04 | 6 |
| ATOM | 6153 | C | VAL | C | 336 | 45.732 | 106.037 | 10.454 | 1.00 | 19.76 | 6 |
| ATOM | 6154 | O | VAL | C | 336 | 46.918 | 106.156 | 10.190 | 1.00 | 19.67 | 8 |
| ATOM | 6155 | N | GLY | C | 337 | 44.758 | 106.511 | 9.681 | 1.00 | 39.74 | 7 |
| ATOM | 6156 | CA | GLY | C | 337 | 45.048 | 107.181 | 8.424 | 1.00 | 41.07 | 6 |
| ATOM | 6157 | C | GLY | C | 337 | 46.305 | 108.021 | 8.456 | 1.00 | 41.88 | 6 |
| ATOM | 6158 | O | GLY | C | 337 | 46.993 | 108.169 | 7.441 | 1.00 | 41.06 | 8 |
| ATOM | 6159 | N | GLU | C | 338 | 46.589 | 108.580 | 9.629 | 1.00 | 53.46 | 7 |
| ATOM | 6160 | CA | GLU | C | 338 | 47.764 | 109.408 | 9.828 | 1.00 | 55.83 | 6 |
| ATOM | 6161 | CB | GLU | C | 338 | 47.595 | 110.228 | 11.117 | 1.00 | 55.93 | 6 |
| ATOM | 6162 | CG | GLU | C | 338 | 48.522 | 111.446 | 11.269 | 1.00 | 57.68 | 6 |
| ATOM | 6163 | CD | GLU | C | 338 | 48.070 | 112.416 | 12.375 | 1.00 | 59.12 | 6 |
| ATOM | 6164 | OE1 | GLU | C | 338 | 48.862 | 113.313 | 12.738 | 1.00 | 59.71 | 8 |
| ATOM | 6165 | OE2 | GLU | C | 338 | 46.927 | 112.293 | 12.877 | 1.00 | 58.33 | 8 |
| ATOM | 6166 | C | GLU | C | 338 | 48.970 | 108.466 | 9.901 | 1.00 | 56.52 | 6 |
| ATOM | 6167 | O | GLU | C | 338 | 49.940 | 108.642 | 9.164 | 1.00 | 57.30 | 8 |
| ATOM | 6168 | N | LEU | C | 339 | 48.896 | 107.456 | 10.768 | 1.00 | 46.17 | 7 |
| ATOM | 6169 | CA | LEU | C | 339 | 49.972 | 106.470 | 10.919 | 1.00 | 45.76 | 6 |
| ATOM | 6170 | CB | LEU | C | 339 | 49.568 | 105.398 | 11.912 | 1.00 | 29.77 | 6 |
| ATOM | 6171 | CG | LEU | C | 339 | 49.259 | 105.826 | 13.334 | 1.00 | 28.34 | 6 |
| ATOM | 6172 | CD1 | LEU | C | 339 | 48.193 | 104.901 | 13.876 | 1.00 | 27.80 | 6 |
| ATOM | 6173 | CD2 | LEU | C | 339 | 50.520 | 105.804 | 14.183 | 1.00 | 26.92 | 6 |
| ATOM | 6174 | C | LEU | C | 339 | 50.247 | 105.785 | 9.593 | 1.00 | 46.77 | 6 |
| ATOM | 6175 | O | LEU | C | 339 | 51.394 | 105.660 | 9.172 | 1.00 | 47.55 | 8 |
| ATOM | 6176 | N | MET | C | 340 | 49.170 | 105.319 | 8.966 | 1.00 | 40.41 | 7 |
| ATOM | 6177 | CA | MET | C | 340 | 49.213 | 104.642 | 7.675 | 1.00 | 41.73 | 6 |
| ATOM | 6178 | CB | MET | C | 340 | 47.815 | 104.102 | 7.357 | 1.00 | 33.54 | 6 |
| ATOM | 6179 | CG | MET | C | 340 | 47.726 | 103.281 | 6.098 | 1.00 | 34.95 | 6 |
| ATOM | 6180 | SD | MET | C | 340 | 47.982 | 104.255 | 4.606 | 1.00 | 37.58 | 16 |
| ATOM | 6181 | CE | MET | C | 340 | 46.303 | 104.575 | 4.124 | 1.00 | 37.44 | 6 |
| ATOM | 6182 | C | MET | C | 340 | 49.685 | 105.624 | 6.585 | 1.00 | 42.70 | 6 |
| ATOM | 6183 | O | MET | C | 340 | 50.313 | 105.237 | 5.590 | 1.00 | 41.52 | 8 |
| ATOM | 6184 | N | ALA | C | 341 | 49.379 | 106.902 | 6.782 | 1.00 | 58.39 | 7 |
| ATOM | 6185 | CA | ALA | C | 341 | 49.783 | 107.922 | 5.832 | 1.00 | 59.26 | 6 |
| ATOM | 6186 | CB | ALA | C | 341 | 49.214 | 109.275 | 6.234 | 1.00 | 54.63 | 6 |
| ATOM | 6187 | C | ALA | C | 341 | 51.302 | 107.975 | 5.808 | 1.00 | 59.78 | 6 |
| ATOM | 6188 | O | ALA | C | 341 | 51.918 | 107.600 | 4.819 | 1.00 | 60.61 | 8 |
| ATOM | 6189 | N | ASP | C | 342 | 51.903 | 108.425 | 6.905 | 1.00 | 54.65 | 7 |
| ATOM | 6190 | CA | ASP | C | 342 | 53.360 | 108.530 | 7.002 | 1.00 | 55.34 | 6 |
| ATOM | 6191 | CB | ASP | C | 342 | 53.785 | 108.750 | 8.459 | 1.00 | 64.26 | 6 |
| ATOM | 6192 | CG | ASP | C | 342 | 53.416 | 110.124 | 8.978 | 1.00 | 65.24 | 6 |
| ATOM | 6193 | OD1 | ASP | C | 342 | 53.659 | 111.125 | 8.266 | 1.00 | 66.05 | 8 |
| ATOM | 6194 | OD2 | ASP | C | 342 | 52.896 | 110.201 | 10.112 | 1.00 | 65.46 | 8 |
| ATOM | 6195 | C | ASP | C | 342 | 54.121 | 107.320 | 6.447 | 1.00 | 55.56 | 6 |
| ATOM | 6196 | O | ASP | C | 342 | 55.191 | 107.467 | 5.850 | 1.00 | 54.83 | 8 |
| ATOM | 6197 | N | GLN | C | 343 | 53.573 | 106.128 | 6.656 | 1.00 | 49.93 | 7 |
| ATOM | 6198 | CA | GLN | C | 343 | 54.217 | 104.916 | 6.183 | 1.00 | 49.77 | 6 |
| ATOM | 6199 | CB | GLN | C | 343 | 53.488 | 103.694 | 6.731 | 1.00 | 55.92 | 6 |
| ATOM | 6200 | CG | GLN | C | 343 | 54.392 | 102.489 | 6.863 | 1.00 | 56.26 | 6 |
| ATOM | 6201 | CD | GLN | C | 343 | 55.600 | 102.773 | 7.735 | 1.00 | 55.71 | 6 |
| ATOM | 6202 | OE1 | GLN | C | 343 | 55.526 | 102.743 | 8.965 | 1.00 | 54.57 | 8 |
| ATOM | 6203 | NE2 | GLN | C | 343 | 56.722 | 103.072 | 7.094 | 1.00 | 55.01 | 7 |
| ATOM | 6204 | C | GLN | C | 343 | 54.201 | 104.903 | 4.663 | 1.00 | 50.19 | 6 |
| ATOM | 6205 | O | GLN | C | 343 | 55.062 | 104.299 | 4.019 | 1.00 | 50.28 | 8 |
| ATOM | 6206 | N | PHE | C | 344 | 53.201 | 105.575 | 4.102 | 1.00 | 68.25 | 7 |
| ATOM | 6207 | CA | PHE | C | 344 | 53.042 | 105.695 | 2.657 | 1.00 | 68.99 | 6 |
| ATOM | 6208 | CB | PHE | C | 344 | 51.628 | 106.199 | 2.331 | 1.00 | 53.22 | 6 |
| ATOM | 6209 | CG | PHE | C | 344 | 51.092 | 105.758 | 0.986 | 1.00 | 52.79 | 6 |
| ATOM | 6210 | CD1 | PHE | C | 344 | 49.781 | 106.061 | 0.620 | 1.00 | 53.28 | 6 |
| ATOM | 6211 | CD2 | PHE | C | 344 | 51.870 | 105.031 | 0.106 | 1.00 | 52.38 | 6 |
| ATOM | 6212 | CE1 | PHE | C | 344 | 49.257 | 105.648 | −0.589 | 1.00 | 53.18 | 6 |
| ATOM | 6213 | CE2 | PHE | C | 344 | 51.350 | 104.614 | −1.107 | 1.00 | 52.97 | 6 |
| ATOM | 6214 | CZ | PHE | C | 344 | 50.039 | 104.925 | −1.452 | 1.00 | 53.08 | 6 |
| ATOM | 6215 | C | PHE | C | 344 | 54.088 | 106.735 | 2.263 | 1.00 | 69.69 | 6 |
| ATOM | 6216 | O | PHE | C | 344 | 54.635 | 106.701 | 1.165 | 1.00 | 70.88 | 8 |
| ATOM | 6217 | N | ARG | C | 345 | 54.359 | 107.663 | 3.177 | 1.00 | 46.04 | 7 |
| ATOM | 6218 | CA | ARG | C | 345 | 55.365 | 108.684 | 2.937 | 1.00 | 46.35 | 6 |
| ATOM | 6219 | CB | ARG | C | 345 | 55.367 | 109.716 | 4.068 | 1.00 | 60.38 | 6 |
| ATOM | 6220 | CG | ARG | C | 345 | 54.080 | 110.523 | 4.148 | 1.00 | 61.90 | 6 |
| ATOM | 6221 | CD | ARG | C | 345 | 54.147 | 111.609 | 5.213 | 1.00 | 62.12 | 6 |
| ATOM | 6222 | NE | ARG | C | 345 | 52.979 | 112.495 | 5.186 | 1.00 | 61.97 | 7 |
| ATOM | 6223 | CZ | ARG | C | 345 | 51.801 | 112.224 | 5.747 | 1.00 | 61.97 | 6 |
| ATOM | 6224 | NH1 | ARG | C | 345 | 51.617 | 111.081 | 6.396 | 1.00 | 61.31 | 7 |
| ATOM | 6225 | NH2 | ARG | C | 345 | 50.800 | 113.096 | 5.656 | 1.00 | 62.32 | 7 |
| ATOM | 6226 | C | ARG | C | 345 | 56.701 | 107.954 | 2.879 | 1.00 | 46.29 | 6 |
| ATOM | 6227 | O | ARG | C | 345 | 57.502 | 108.172 | 1.974 | 1.00 | 45.82 | 8 |
| ATOM | 6228 | N | VAL | C | 346 | 56.917 | 107.059 | 3.837 | 1.00 | 59.02 | 7 |
| ATOM | 6229 | CA | VAL | C | 346 | 58.148 | 106.272 | 3.909 | 1.00 | 59.39 | 6 |
| ATOM | 6230 | CB | VAL | C | 346 | 58.072 | 105.227 | 5.042 | 1.00 | 106.13 | 6 |
| ATOM | 6231 | CG1 | VAL | C | 346 | 59.400 | 104.491 | 5.167 | 1.00 | 106.98 | 6 |
| ATOM | 6232 | CG2 | VAL | C | 346 | 57.705 | 105.908 | 6.348 | 1.00 | 106.03 | 6 |
| ATOM | 6233 | C | VAL | C | 346 | 58.418 | 105.533 | 2.603 | 1.00 | 59.04 | 6 |
| ATOM | 6234 | O | VAL | C | 346 | 59.521 | 105.588 | 2.059 | 1.00 | 58.47 | 8 |
| ATOM | 6235 | N | GLY | C | 347 | 57.402 | 104.830 | 2.113 | 1.00 | 36.64 | 7 |
| ATOM | 6236 | CA | GLY | C | 347 | 57.550 | 104.086 | 0.880 | 1.00 | 38.67 | 6 |
| ATOM | 6237 | C | GLY | C | 347 | 58.151 | 104.955 | −0.197 | 1.00 | 40.04 | 6 |
| ATOM | 6238 | O | GLY | C | 347 | 59.142 | 104.591 | −0.818 | 1.00 | 38.54 | 8 |
| ATOM | 6239 | N | LEU | C | 348 | 57.550 | 106.116 | −0.410 | 1.00 | 81.14 | 7 |
| ATOM | 6240 | CA | LEU | C | 348 | 58.032 | 107.041 | −1.415 | 1.00 | 83.72 | 6 |
| ATOM | 6241 | CB | LEU | C | 348 | 57.045 | 108.198 | −1.557 | 1.00 | 23.40 | 6 |
| ATOM | 6242 | CG | LEU | C | 348 | 55.937 | 107.959 | −2.591 | 1.00 | 21.23 | 6 |
| ATOM | 6243 | CD1 | LEU | C | 348 | 55.523 | 106.507 | −2.605 | 1.00 | 21.40 | 6 |
| ATOM | 6244 | CD2 | LEU | C | 348 | 54.750 | 108.847 | −2.277 | 1.00 | 20.29 | 6 |
| ATOM | 6245 | C | LEU | C | 348 | 59.430 | 107.551 | −1.088 | 1.00 | 87.02 | 6 |
| ATOM | 6246 | O | LEU | C | 348 | 60.176 | 107.943 | −1.982 | 1.00 | 87.49 | 8 |
| ATOM | 6247 | N | ALA | C | 349 | 59.794 | 107.536 | 0.190 | 1.00 | 205.10 | 7 |
| ATOM | 6248 | CA | ALA | C | 349 | 61.122 | 107.987 | 0.597 | 1.00 | 208.87 | 6 |
| ATOM | 6249 | CB | ALA | C | 349 | 61.181 | 108.163 | 2.107 | 1.00 | 160.69 | 6 |
| ATOM | 6250 | C | ALA | C | 349 | 62.125 | 106.933 | 0.157 | 1.00 | 208.87 | 6 |
| ATOM | 6251 | O | ALA | C | 349 | 63.265 | 106.903 | 0.622 | 1.00 | 208.87 | 8 |
| ATOM | 6252 | N | ARG | C | 350 | 61.680 | 106.069 | −0.751 | 1.00 | 62.51 | 7 |
| ATOM | 6253 | CA | ARG | C | 350 | 62.495 | 104.983 | −1.264 | 1.00 | 63.53 | 6 |
| ATOM | 6254 | CB | ARG | C | 350 | 62.248 | 103.770 | −0.392 | 1.00 | 74.24 | 6 |
| ATOM | 6255 | CG | ARG | C | 350 | 62.658 | 104.075 | 1.026 | 1.00 | 75.44 | 6 |
| ATOM | 6256 | CD | ARG | C | 350 | 61.948 | 103.257 | 2.077 | 1.00 | 75.95 | 6 |
| ATOM | 6257 | NE | ARG | C | 350 | 62.617 | 103.445 | 3.360 | 1.00 | 76.40 | 7 |
| ATOM | 6258 | CZ | ARG | C | 350 | 62.274 | 102.835 | 4.485 | 1.00 | 77.84 | 6 |
| ATOM | 6259 | NH1 | ARG | C | 350 | 61.251 | 101.987 | 4.500 | 1.00 | 78.67 | 7 |
| ATOM | 6260 | NH2 | ARG | C | 350 | 62.971 | 103.061 | 5.589 | 1.00 | 79.33 | 7 |
| ATOM | 6261 | C | ARG | C | 350 | 62.158 | 104.712 | −2.727 | 1.00 | 64.82 | 6 |
| ATOM | 6262 | O | ARG | C | 350 | 63.020 | 104.347 | −3.525 | 1.00 | 65.28 | 8 |
| ATOM | 6263 | N | LEU | C | 351 | 60.891 | 104.890 | −3.072 | 1.00 | 63.44 | 7 |
| ATOM | 6264 | CA | LEU | C | 351 | 60.462 | 104.716 | −4.444 | 1.00 | 64.60 | 6 |
| ATOM | 6265 | CB | LEU | C | 351 | 59.025 | 105.189 | −4.612 | 1.00 | 69.32 | 6 |
| ATOM | 6266 | CG | LEU | C | 351 | 58.740 | 105.890 | −5.937 | 1.00 | 69.81 | 6 |
| ATOM | 6267 | CD1 | LEU | C | 351 | 58.831 | 104.902 | −7.077 | 1.00 | 70.58 | 6 |
| ATOM | 6268 | CD2 | LEU | C | 351 | 57.374 | 106.517 | −5.887 | 1.00 | 69.70 | 6 |
| ATOM | 6269 | C | LEU | C | 351 | 61.379 | 105.643 | −5.196 | 1.00 | 65.69 | 6 |
| ATOM | 6270 | O | LEU | C | 351 | 62.157 | 105.214 | −6.032 | 1.00 | 66.02 | 8 |
| ATOM | 6271 | N | ALA | C | 352 | 61.290 | 106.924 | −4.856 | 1.00 | 97.21 | 7 |
| ATOM | 6272 | CA | ALA | C | 352 | 62.096 | 107.968 | −5.477 | 1.00 | 98.34 | 6 |
| ATOM | 6273 | CB | ALA | C | 352 | 61.961 | 109.260 | −4.693 | 1.00 | 107.71 | 6 |
| ATOM | 6274 | C | ALA | C | 352 | 63.566 | 107.581 | −5.598 | 1.00 | 98.88 | 6 |
| ATOM | 6275 | O | ALA | C | 352 | 64.119 | 107.579 | −6.698 | 1.00 | 99.53 | 8 |
| ATOM | 6276 | N | ARG | C | 353 | 64.208 | 107.271 | −4.473 | 1.00 | 45.94 | 7 |
| ATOM | 6277 | CA | ARG | C | 353 | 65.609 | 106.871 | −4.514 | 1.00 | 47.38 | 6 |
| ATOM | 6278 | CB | ARG | C | 353 | 65.973 | 106.085 | −3.254 | 1.00 | 115.76 | 6 |
| ATOM | 6279 | CG | ARG | C | 353 | 65.775 | 106.841 | −1.955 | 1.00 | 117.15 | 6 |
| ATOM | 6280 | CD | ARG | C | 353 | 66.825 | 107.914 | −1.782 | 1.00 | 118.82 | 6 |
| ATOM | 6281 | NE | ARG | C | 353 | 66.625 | 108.662 | −0.546 | 1.00 | 120.11 | 7 |
| ATOM | 6282 | CZ | ARG | C | 353 | 67.444 | 109.613 | −0.108 | 1.00 | 121.05 | 6 |
| ATOM | 6283 | NH1 | ARG | C | 353 | 68.529 | 109.935 | −0.805 | 1.00 | 120.67 | 7 |
| ATOM | 6284 | NH2 | ARG | C | 353 | 67.177 | 110.245 | 1.028 | 1.00 | 121.41 | 7 |
| ATOM | 6285 | C | ARG | C | 353 | 65.763 | 105.972 | −5.742 | 1.00 | 48.30 | 6 |
| ATOM | 6286 | O | ARG | C | 353 | 66.610 | 106.199 | −6.605 | 1.00 | 49.16 | 8 |
| ATOM | 6287 | N | GLY | C | 354 | 64.908 | 104.959 | −5.820 | 1.00 | 160.51 | 7 |
| ATOM | 6288 | CA | GLY | C | 354 | 64.955 | 104.049 | −6.944 | 1.00 | 160.56 | 6 |
| ATOM | 6289 | C | GLY | C | 354 | 64.848 | 104.787 | −8.263 | 1.00 | 160.66 | 6 |
| ATOM | 6290 | O | GLY | C | 354 | 65.679 | 104.601 | −9.152 | 1.00 | 161.53 | 8 |
| ATOM | 6291 | N | VAL | C | 355 | 63.835 | 105.638 | −8.395 | 1.00 | 80.66 | 7 |
| ATOM | 6292 | CA | VAL | C | 355 | 63.653 | 106.371 | −9.633 | 1.00 | 80.30 | 6 |
| ATOM | 6293 | CB | VAL | C | 355 | 62.332 | 107.204 | −9.622 | 1.00 | 45.69 | 6 |
| ATOM | 6294 | CG1 | VAL | C | 355 | 62.416 | 108.360 | −8.651 | 1.00 | 45.97 | 6 |
| ATOM | 6295 | CG2 | VAL | C | 355 | 62.024 | 107.704 | −11.027 | 1.00 | 45.21 | 6 |
| ATOM | 6296 | C | VAL | C | 355 | 64.861 | 107.258 | −9.939 | 1.00 | 80.64 | 6 |
| ATOM | 6297 | O | VAL | C | 355 | 65.468 | 107.114 | −10.998 | 1.00 | 81.25 | 8 |
| ATOM | 6298 | N | ARG | C | 356 | 65.226 | 108.152 | −9.022 | 1.00 | 80.64 | 7 |
| ATOM | 6299 | CA | ARG | C | 356 | 66.383 | 109.022 | −9.238 | 1.00 | 80.31 | 6 |
| ATOM | 6300 | CB | ARG | C | 356 | 66.842 | 109.659 | −7.923 | 1.00 | 101.69 | 6 |
| ATOM | 6301 | CG | ARG | C | 356 | 68.368 | 109.649 | −7.714 | 1.00 | 101.76 | 6 |
| ATOM | 6302 | CD | ARG | C | 356 | 69.130 | 110.592 | −8.662 | 1.00 | 102.00 | 6 |
| ATOM | 6303 | NE | ARG | C | 356 | 70.579 | 110.374 | −8.601 | 1.00 | 101.48 | 7 |
| ATOM | 6304 | CZ | ARG | C | 356 | 71.496 | 111.201 | −9.105 | 1.00 | 101.01 | 6 |
| ATOM | 6305 | NH1 | ARG | C | 356 | 71.127 | 112.321 | −9.715 | 1.00 | 100.84 | 7 |
| ATOM | 6306 | NH2 | ARG | C | 356 | 72.787 | 110.906 | −8.997 | 1.00 | 101.02 | 7 |
| ATOM | 6307 | C | ARG | C | 356 | 67.522 | 108.191 | −9.800 | 1.00 | 80.38 | 6 |
| ATOM | 6308 | O | ARG | C | 356 | 68.235 | 108.631 | −10.702 | 1.00 | 79.87 | 8 |
| ATOM | 6309 | N | GLU | C | 357 | 67.685 | 106.993 | −9.241 | 1.00 | 70.79 | 7 |
| ATOM | 6310 | CA | GLU | C | 357 | 68.723 | 106.060 | −9.654 | 1.00 | 71.20 | 6 |
| ATOM | 6311 | CB | GLU | C | 357 | 68.626 | 104.772 | −8.836 | 1.00 | 197.19 | 6 |
| ATOM | 6312 | CG | GLU | C | 357 | 69.548 | 103.661 | −9.312 | 1.00 | 199.52 | 6 |
| ATOM | 6313 | CD | GLU | C | 357 | 71.008 | 103.958 | −9.048 | 1.00 | 200.44 | 6 |
| ATOM | 6314 | OE1 | GLU | C | 357 | 71.479 | 105.043 | −9.451 | 1.00 | 202.08 | 8 |
| ATOM | 6315 | OE2 | GLU | C | 357 | 71.687 | 103.101 | −8.441 | 1.00 | 200.31 | 8 |
| ATOM | 6316 | C | GLU | C | 357 | 68.588 | 105.724 | −11.128 | 1.00 | 70.90 | 6 |
| ATOM | 6317 | O | GLU | C | 357 | 69.535 | 105.887 | −11.891 | 1.00 | 70.88 | 8 |
| ATOM | 6318 | N | ARG | C | 358 | 67.405 | 105.257 | −11.521 | 1.00 | 98.92 | 7 |
| ATOM | 6319 | CA | ARG | C | 358 | 67.143 | 104.884 | −12.911 | 1.00 | 99.88 | 6 |
| ATOM | 6320 | CB | ARG | C | 358 | 65.758 | 104.240 | −13.040 | 1.00 | 90.72 | 6 |
| ATOM | 6321 | CG | ARG | C | 358 | 65.413 | 103.224 | −11.957 | 1.00 | 90.05 | 6 |
| ATOM | 6322 | CD | ARG | C | 358 | 66.334 | 102.006 | −11.935 | 1.00 | 88.23 | 6 |
| ATOM | 6323 | NE | ARG | C | 358 | 65.958 | 101.101 | −10.849 | 1.00 | 86.36 | 7 |
| ATOM | 6324 | CZ | ARG | C | 358 | 66.613 | 99.992 | −10.520 | 1.00 | 85.41 | 6 |
| ATOM | 6325 | NH1 | ARG | C | 358 | 67.696 | 99.623 | −11.189 | 1.00 | 84.88 | 7 |
| ATOM | 6326 | NH2 | ARG | C | 358 | 66.181 | 99.250 | −9.513 | 1.00 | 85.19 | 7 |
| ATOM | 6327 | C | ARG | C | 358 | 67.229 | 106.086 | −13.855 | 1.00 | 101.13 | 6 |
| ATOM | 6328 | O | ARG | C | 358 | 67.926 | 106.033 | −14.871 | 1.00 | 101.33 | 8 |
| ATOM | 6329 | N | MET | C | 359 | 66.508 | 107.155 | −13.521 | 1.00 | 78.58 | 7 |
| ATOM | 6330 | CA | MET | C | 359 | 66.510 | 108.377 | −14.326 | 1.00 | 80.42 | 6 |
| ATOM | 6331 | CB | MET | C | 359 | 66.186 | 109.607 | −13.457 | 1.00 | 98.08 | 6 |
| ATOM | 6332 | CG | MET | C | 359 | 64.756 | 109.687 | −12.898 | 1.00 | 100.34 | 6 |
| ATOM | 6333 | SD | MET | C | 359 | 63.524 | 110.434 | −14.000 | 1.00 | 101.54 | 16 |
| ATOM | 6334 | CE | MET | C | 359 | 63.522 | 112.080 | −13.421 | 1.00 | 101.52 | 6 |
| ATOM | 6335 | C | MET | C | 359 | 67.886 | 108.561 | −14.951 | 1.00 | 81.29 | 6 |
| ATOM | 6336 | O | MET | C | 359 | 68.002 | 108.834 | −16.141 | 1.00 | 81.42 | 8 |
| ATOM | 6337 | N | VAL | C | 360 | 68.926 | 108.397 | −14.138 | 1.00 | 137.54 | 7 |
| ATOM | 6338 | CA | VAL | C | 360 | 70.300 | 108.547 | −14.600 | 1.00 | 139.21 | 6 |
| ATOM | 6339 | CB | VAL | C | 360 | 71.267 | 108.621 | −13.408 | 1.00 | 61.42 | 6 |
| ATOM | 6340 | CG1 | VAL | C | 360 | 72.711 | 108.678 | −13.883 | 1.00 | 61.29 | 6 |
| ATOM | 6341 | CG2 | VAL | C | 360 | 70.942 | 109.837 | −12.591 | 1.00 | 61.94 | 6 |
| ATOM | 6342 | C | VAL | C | 360 | 70.726 | 107.419 | −15.527 | 1.00 | 140.74 | 6 |
| ATOM | 6343 | O | VAL | C | 360 | 70.462 | 107.475 | −16.725 | 1.00 | 141.23 | 8 |
| ATOM | 6344 | N | MET | C | 361 | 71.380 | 106.402 | −14.968 | 1.00 | 105.12 | 7 |
| ATOM | 6345 | CA | MET | C | 361 | 71.857 | 105.263 | −15.743 | 1.00 | 106.42 | 6 |
| ATOM | 6346 | CB | MET | C | 361 | 71.439 | 103.927 | −15.114 | 1.00 | 110.40 | 6 |
| ATOM | 6347 | CG | MET | C | 361 | 71.938 | 103.629 | −13.716 | 1.00 | 112.79 | 6 |
| ATOM | 6348 | SD | MET | C | 361 | 71.479 | 101.941 | −13.236 | 1.00 | 113.05 | 16 |
| ATOM | 6349 | CE | MET | C | 361 | 69.714 | 102.024 | −13.313 | 1.00 | 113.30 | 6 |
| ATOM | 6350 | C | MET | C | 361 | 71.259 | 105.295 | −17.128 | 1.00 | 106.56 | 6 |
| ATOM | 6351 | O | MET | C | 361 | 71.868 | 105.790 | −18.076 | 1.00 | 107.71 | 8 |
| ATOM | 6352 | N | GLY | C | 362 | 70.036 | 104.776 | −17.205 | 1.00 | 77.49 | 7 |
| ATOM | 6353 | CA | GLY | C | 362 | 69.305 | 104.676 | −18.453 | 1.00 | 77.57 | 6 |
| ATOM | 6354 | C | GLY | C | 362 | 69.388 | 105.847 | −19.402 | 1.00 | 77.61 | 6 |
| ATOM | 6355 | O | GLY | C | 362 | 69.930 | 106.897 | −19.070 | 1.00 | 78.30 | 8 |
| ATOM | 6356 | N | SER | C | 363 | 68.842 | 105.650 | −20.597 | 1.00 | 117.73 | 7 |
| ATOM | 6357 | CA | SER | C | 363 | 68.822 | 106.685 | −21.620 | 1.00 | 118.18 | 6 |
| ATOM | 6358 | CB | SER | C | 363 | 68.919 | 106.057 | −23.015 | 1.00 | 165.58 | 6 |
| ATOM | 6359 | OG | SER | C | 363 | 68.963 | 107.047 | −24.028 | 1.00 | 165.87 | 8 |
| ATOM | 6360 | C | SER | C | 363 | 67.511 | 107.450 | −21.487 | 1.00 | 118.23 | 6 |
| ATOM | 6361 | O | SER | C | 363 | 66.501 | 106.897 | −21.058 | 1.00 | 118.26 | 8 |
| ATOM | 6362 | N | PRO | C | 364 | 67.511 | 108.735 | −21.861 | 1.00 | 137.80 | 7 |
| ATOM | 6363 | CD | PRO | C | 364 | 68.608 | 109.457 | −22.525 | 1.00 | 159.94 | 6 |
| ATOM | 6364 | CA | PRO | C | 364 | 66.316 | 109.575 | −21.780 | 1.00 | 137.10 | 6 |
| ATOM | 6365 | CB | PRO | C | 364 | 66.791 | 110.902 | −22.367 | 1.00 | 160.15 | 6 |
| ATOM | 6366 | CG | PRO | C | 364 | 67.859 | 110.479 | −23.332 | 1.00 | 160.69 | 6 |
| ATOM | 6367 | C | PRO | C | 364 | 65.081 | 109.033 | −22.499 | 1.00 | 136.05 | 6 |
| ATOM | 6368 | O | PRO | C | 364 | 63.962 | 109.211 | −22.013 | 1.00 | 136.06 | 8 |
| ATOM | 6369 | N | ASP | C | 365 | 65.275 | 108.375 | −23.643 | 1.00 | 91.02 | 7 |
| ATOM | 6370 | CA | ASP | C | 365 | 64.146 | 107.837 | −24.415 | 1.00 | 89.06 | 6 |
| ATOM | 6371 | CB | ASP | C | 365 | 64.462 | 107.865 | −25.918 | 1.00 | 162.10 | 6 |
| ATOM | 6372 | CG | ASP | C | 365 | 63.205 | 107.890 | −26.783 | 1.00 | 163.67 | 6 |
| ATOM | 6373 | OD1 | ASP | C | 365 | 62.364 | 106.980 | −26.647 | 1.00 | 165.13 | 8 |
| ATOM | 6374 | OD2 | ASP | C | 365 | 63.058 | 108.823 | −27.601 | 1.00 | 164.24 | 8 |
| ATOM | 6375 | C | ASP | C | 365 | 63.768 | 106.416 | −23.994 | 1.00 | 87.01 | 6 |
| ATOM | 6376 | O | ASP | C | 365 | 62.605 | 106.016 | −24.084 | 1.00 | 86.49 | 8 |
| ATOM | 6377 | N | THR | C | 366 | 64.753 | 105.655 | −23.535 | 1.00 | 177.30 | 7 |
| ATOM | 6378 | CA | THR | C | 366 | 64.495 | 104.298 | −23.090 | 1.00 | 175.40 | 6 |
| ATOM | 6379 | CB | THR | C | 366 | 65.800 | 103.518 | −22.886 | 1.00 | 130.83 | 6 |
| ATOM | 6380 | OG1 | THR | C | 366 | 66.573 | 103.560 | −24.091 | 1.00 | 131.14 | 8 |
| ATOM | 6381 | CG2 | THR | C | 366 | 65.504 | 102.069 | −22.527 | 1.00 | 131.24 | 6 |
| ATOM | 6382 | C | THR | C | 366 | 63.766 | 104.385 | −21.759 | 1.00 | 173.92 | 6 |
| ATOM | 6383 | O | THR | C | 366 | 63.205 | 103.402 | −21.282 | 1.00 | 175.00 | 8 |
| ATOM | 6384 | N | LEU | C | 367 | 63.773 | 105.578 | −21.170 | 1.00 | 79.11 | 7 |
| ATOM | 6385 | CA | LEU | C | 367 | 63.127 | 105.812 | −19.884 | 1.00 | 75.66 | 6 |
| ATOM | 6386 | CB | LEU | C | 367 | 63.909 | 106.844 | −19.080 | 1.00 | 69.38 | 6 |
| ATOM | 6387 | CG | LEU | C | 367 | 65.295 | 106.413 | −18.623 | 1.00 | 68.94 | 6 |
| ATOM | 6388 | CD1 | LEU | C | 367 | 65.850 | 107.463 | −17.690 | 1.00 | 68.92 | 6 |
| ATOM | 6389 | CD2 | LEU | C | 367 | 65.214 | 105.068 | −17.918 | 1.00 | 69.19 | 6 |
| ATOM | 6390 | C | LEU | C | 367 | 61.692 | 106.281 | −19.989 | 1.00 | 73.59 | 6 |
| ATOM | 6391 | O | LEU | C | 367 | 61.388 | 107.221 | −20.713 | 1.00 | 73.53 | 8 |
| ATOM | 6392 | N | THR | C | 368 | 60.813 | 105.620 | −19.249 | 1.00 | 96.65 | 7 |
| ATOM | 6393 | CA | THR | C | 368 | 59.402 | 105.973 | −19.224 | 1.00 | 94.95 | 6 |
| ATOM | 6394 | CB | THR | C | 368 | 58.624 | 105.319 | −20.382 | 1.00 | 107.98 | 6 |
| ATOM | 6395 | OG1 | THR | C | 368 | 58.611 | 103.899 | −20.207 | 1.00 | 107.29 | 8 |
| ATOM | 6396 | CG2 | THR | C | 368 | 59.261 | 105.655 | −21.717 | 1.00 | 108.58 | 6 |
| ATOM | 6397 | C | THR | C | 368 | 58.837 | 105.450 | −17.910 | 1.00 | 93.31 | 6 |
| ATOM | 6398 | O | THR | C | 368 | 59.460 | 104.619 | −17.245 | 1.00 | 94.32 | 8 |
| ATOM | 6399 | N | PRO | C | 369 | 57.655 | 105.935 | −17.508 | 1.00 | 69.12 | 7 |
| ATOM | 6400 | CD | PRO | C | 369 | 56.695 | 106.779 | −18.229 | 1.00 | 28.46 | 6 |
| ATOM | 6401 | CA | PRO | C | 369 | 57.075 | 105.451 | −16.255 | 1.00 | 67.49 | 6 |
| ATOM | 6402 | CB | PRO | C | 369 | 55.753 | 106.202 | −16.178 | 1.00 | 28.00 | 6 |
| ATOM | 6403 | CG | PRO | C | 369 | 55.401 | 106.382 | −17.583 | 1.00 | 27.23 | 6 |
| ATOM | 6404 | C | PRO | C | 369 | 56.882 | 103.953 | −16.385 | 1.00 | 66.12 | 6 |
| ATOM | 6405 | O | PRO | C | 369 | 57.017 | 103.401 | −17.476 | 1.00 | 66.14 | 8 |
| ATOM | 6406 | N | ALA | C | 370 | 56.564 | 103.292 | −15.283 | 1.00 | 93.49 | 7 |
| ATOM | 6407 | CA | ALA | C | 370 | 56.382 | 101.851 | −15.314 | 1.00 | 91.62 | 6 |
| ATOM | 6408 | CB | ALA | C | 370 | 55.515 | 101.446 | −16.497 | 1.00 | 18.84 | 6 |
| ATOM | 6409 | C | ALA | C | 370 | 57.754 | 101.223 | −15.444 | 1.00 | 90.43 | 6 |
| ATOM | 6410 | O | ALA | C | 370 | 57.928 | 100.042 | −15.154 | 1.00 | 89.81 | 8 |
| ATOM | 6411 | N | LYS | C | 371 | 58.729 | 102.023 | −15.874 | 1.00 | 47.06 | 7 |
| ATOM | 6412 | CA | LYS | C | 371 | 60.089 | 101.532 | −16.046 | 1.00 | 46.49 | 6 |
| ATOM | 6413 | CB | LYS | C | 371 | 60.581 | 101.857 | −17.458 | 1.00 | 103.72 | 6 |
| ATOM | 6414 | CG | LYS | C | 371 | 59.697 | 101.217 | −18.521 | 1.00 | 104.63 | 6 |
| ATOM | 6415 | CD | LYS | C | 371 | 60.192 | 101.434 | −19.935 | 1.00 | 104.61 | 6 |
| ATOM | 6416 | CE | LYS | C | 371 | 59.220 | 100.810 | −20.932 | 1.00 | 105.32 | 6 |
| ATOM | 6417 | NZ | LYS | C | 371 | 57.824 | 101.341 | −20.797 | 1.00 | 103.82 | 7 |
| ATOM | 6418 | C | LYS | C | 371 | 61.057 | 102.057 | −14.995 | 1.00 | 45.54 | 6 |
| ATOM | 6419 | O | LYS | C | 371 | 61.917 | 101.306 | −14.512 | 1.00 | 44.46 | 8 |
| ATOM | 6420 | N | LEU | C | 372 | 60.909 | 103.338 | −14.645 | 1.00 | 69.08 | 7 |
| ATOM | 6421 | CA | LEU | C | 372 | 61.749 | 103.989 | −13.624 | 1.00 | 68.75 | 6 |
| ATOM | 6422 | CB | LEU | C | 372 | 61.683 | 105.522 | −13.739 | 1.00 | 76.05 | 6 |
| ATOM | 6423 | CG | LEU | C | 372 | 62.355 | 106.304 | −14.874 | 1.00 | 75.32 | 6 |
| ATOM | 6424 | CD1 | LEU | C | 372 | 61.856 | 107.738 | −14.834 | 1.00 | 74.70 | 6 |
| ATOM | 6425 | CD2 | LEU | C | 372 | 63.874 | 106.257 | −14.740 | 1.00 | 76.45 | 6 |
| ATOM | 6426 | C | LEU | C | 372 | 61.180 | 103.592 | −12.274 | 1.00 | 67.85 | 6 |
| ATOM | 6427 | O | LEU | C | 372 | 61.875 | 103.610 | −11.253 | 1.00 | 66.75 | 8 |
| ATOM | 6428 | N | VAL | C | 373 | 59.894 | 103.248 | −12.307 | 1.00 | 58.10 | 7 |
| ATOM | 6429 | CA | VAL | C | 373 | 59.124 | 102.836 | −11.146 | 1.00 | 57.39 | 6 |
| ATOM | 6430 | CB | VAL | C | 373 | 57.690 | 103.234 | −11.324 | 1.00 | 57.10 | 6 |
| ATOM | 6431 | CG1 | VAL | C | 373 | 56.922 | 102.957 | −10.059 | 1.00 | 57.34 | 6 |
| ATOM | 6432 | CG2 | VAL | C | 373 | 57.621 | 104.691 | −11.740 | 1.00 | 56.99 | 6 |
| ATOM | 6433 | C | VAL | C | 373 | 59.174 | 101.329 | −11.000 | 1.00 | 58.00 | 6 |
| ATOM | 6434 | O | VAL | C | 373 | 59.004 | 100.599 | −11.978 | 1.00 | 58.26 | 8 |
| ATOM | 6435 | N | ASN | C | 374 | 59.390 | 100.862 | −9.774 | 1.00 | 33.15 | 7 |
| ATOM | 6436 | CA | ASN | C | 374 | 59.493 | 99.425 | −9.516 | 1.00 | 34.29 | 6 |
| ATOM | 6437 | CB | ASN | C | 374 | 60.964 | 99.025 | −9.321 | 1.00 | 128.77 | 6 |
| ATOM | 6438 | CG | ASN | C | 374 | 61.844 | 99.414 | −10.501 | 1.00 | 130.53 | 6 |
| ATOM | 6439 | OD1 | ASN | C | 374 | 61.560 | 99.063 | −11.648 | 1.00 | 132.45 | 8 |
| ATOM | 6440 | ND2 | ASN | C | 374 | 62.927 | 100.132 | −10.219 | 1.00 | 130.55 | 7 |
| ATOM | 6441 | C | ASN | C | 374 | 58.695 | 98.997 | −8.295 | 1.00 | 34.29 | 6 |
| ATOM | 6442 | O | ASN | C | 374 | 59.224 | 98.306 | −7.424 | 1.00 | 33.45 | 8 |
| ATOM | 6443 | N | SER | C | 375 | 57.427 | 99.416 | −8.266 | 1.00 | 44.69 | 7 |
| ATOM | 6444 | CA | SER | C | 375 | 56.444 | 99.141 | −7.210 | 1.00 | 46.27 | 6 |
| ATOM | 6445 | CB | SER | C | 375 | 55.100 | 98.822 | −7.863 | 1.00 | 208.87 | 6 |
| ATOM | 6446 | OG | SER | C | 375 | 54.709 | 99.864 | −8.744 | 1.00 | 208.87 | 8 |
| ATOM | 6447 | C | SER | C | 375 | 56.778 | 98.049 | −6.196 | 1.00 | 46.12 | 6 |
| ATOM | 6448 | O | SER | C | 375 | 56.040 | 97.066 | −6.062 | 1.00 | 45.87 | 8 |
| ATOM | 6449 | N | ARG | C | 376 | 57.879 | 98.255 | −5.475 | 1.00 | 32.74 | 7 |
| ATOM | 6450 | CA | ARG | C | 376 | 58.375 | 97.334 | −4.458 | 1.00 | 32.98 | 6 |
| ATOM | 6451 | CB | ARG | C | 376 | 59.766 | 96.821 | −4.840 | 1.00 | 142.46 | 6 |
| ATOM | 6452 | CG | ARG | C | 376 | 59.794 | 95.829 | −5.999 | 1.00 | 146.04 | 6 |
| ATOM | 6453 | CD | ARG | C | 376 | 59.085 | 94.530 | −5.637 | 1.00 | 147.74 | 6 |
| ATOM | 6454 | NE | ARG | C | 376 | 59.292 | 93.491 | −6.642 | 1.00 | 149.33 | 7 |
| ATOM | 6455 | CZ | ARG | C | 376 | 58.718 | 92.292 | −6.604 | 1.00 | 149.85 | 6 |
| ATOM | 6456 | NH1 | ARG | C | 376 | 57.895 | 91.977 | −5.612 | 1.00 | 150.47 | 7 |
| ATOM | 6457 | NH2 | ARG | C | 376 | 58.967 | 91.406 | −7.558 | 1.00 | 149.73 | 7 |
| ATOM | 6458 | C | ARG | C | 376 | 58.453 | 98.018 | −3.101 | 1.00 | 32.39 | 6 |
| ATOM | 6459 | O | ARG | C | 376 | 58.211 | 97.395 | −2.076 | 1.00 | 31.97 | 8 |
| ATOM | 6460 | N | PRO | C | 377 | 58.804 | 99.314 | −3.074 | 1.00 | 46.43 | 7 |
| ATOM | 6461 | CD | PRO | C | 377 | 59.159 | 100.179 | −4.213 | 1.00 | 162.38 | 6 |
| ATOM | 6462 | CA | PRO | C | 377 | 58.906 | 100.066 | −1.819 | 1.00 | 45.85 | 6 |
| ATOM | 6463 | CB | PRO | C | 377 | 59.516 | 101.392 | −2.261 | 1.00 | 161.93 | 6 |
| ATOM | 6464 | CG | PRO | C | 377 | 58.963 | 101.562 | −3.635 | 1.00 | 162.91 | 6 |
| ATOM | 6465 | C | PRO | C | 377 | 57.576 | 100.259 | −1.103 | 1.00 | 44.99 | 6 |
| ATOM | 6466 | O | PRO | C | 377 | 57.491 | 100.043 | 0.101 | 1.00 | 44.13 | 8 |
| ATOM | 6467 | N | LEU | C | 378 | 56.544 | 100.675 | −1.830 | 1.00 | 64.77 | 7 |
| ATOM | 6468 | CA | LEU | C | 378 | 55.242 | 100.876 | −1.203 | 1.00 | 64.97 | 6 |
| ATOM | 6469 | CB | LEU | C | 378 | 54.240 | 101.449 | −2.199 | 1.00 | 83.43 | 6 |
| ATOM | 6470 | CG | LEU | C | 378 | 54.400 | 102.963 | −2.337 | 1.00 | 84.69 | 6 |
| ATOM | 6471 | CD1 | LEU | C | 378 | 55.849 | 103.294 | −2.676 | 1.00 | 85.16 | 6 |
| ATOM | 6472 | CD2 | LEU | C | 378 | 53.455 | 103.489 | −3.402 | 1.00 | 84.91 | 6 |
| ATOM | 6473 | C | LEU | C | 378 | 54.722 | 99.581 | −0.605 | 1.00 | 64.76 | 6 |
| ATOM | 6474 | O | LEU | C | 378 | 53.809 | 99.588 | 0.214 | 1.00 | 65.34 | 8 |
| ATOM | 6475 | N | GLU | C | 379 | 55.296 | 98.466 | −1.038 | 1.00 | 42.46 | 7 |
| ATOM | 6476 | CA | GLU | C | 379 | 54.943 | 97.166 | −0.497 | 1.00 | 41.70 | 6 |
| ATOM | 6477 | CB | GLU | C | 379 | 54.995 | 96.091 | −1.580 | 1.00 | 100.82 | 6 |
| ATOM | 6478 | CG | GLU | C | 379 | 54.363 | 94.776 | −1.173 | 1.00 | 103.75 | 6 |
| ATOM | 6479 | CD | GLU | C | 379 | 52.895 | 94.920 | −0.812 | 1.00 | 105.94 | 6 |
| ATOM | 6480 | OE1 | GLU | C | 379 | 52.151 | 95.538 | −1.601 | 1.00 | 107.50 | 8 |
| ATOM | 6481 | OE2 | GLU | C | 379 | 52.481 | 94.409 | 0.254 | 1.00 | 107.21 | 8 |
| ATOM | 6482 | C | GLU | C | 379 | 56.117 | 97.033 | 0.457 | 1.00 | 40.89 | 6 |
| ATOM | 6483 | O | GLU | C | 379 | 57.064 | 97.811 | 0.365 | 1.00 | 40.81 | 8 |
| ATOM | 6484 | N | ALA | C | 380 | 56.085 | 96.072 | 1.365 | 1.00 | 100.07 | 7 |
| ATOM | 6485 | CA | ALA | C | 380 | 57.179 | 95.951 | 2.319 | 1.00 | 98.75 | 6 |
| ATOM | 6486 | CB | ALA | C | 380 | 58.546 | 96.036 | 1.603 | 1.00 | 32.13 | 6 |
| ATOM | 6487 | C | ALA | C | 380 | 57.030 | 97.111 | 3.299 | 1.00 | 97.64 | 6 |
| ATOM | 6488 | O | ALA | C | 380 | 56.416 | 96.973 | 4.352 | 1.00 | 97.70 | 8 |
| ATOM | 6489 | N | ALA | C | 381 | 57.568 | 98.267 | 2.929 | 1.00 | 64.15 | 7 |
| ATOM | 6490 | CA | ALA | C | 381 | 57.505 | 99.443 | 3.788 | 1.00 | 63.00 | 6 |
| ATOM | 6491 | CB | ALA | C | 381 | 58.055 | 100.662 | 3.053 | 1.00 | 95.29 | 6 |
| ATOM | 6492 | C | ALA | C | 381 | 56.089 | 99.711 | 4.259 | 1.00 | 62.40 | 6 |
| ATOM | 6493 | O | ALA | C | 381 | 55.882 | 100.389 | 5.260 | 1.00 | 62.60 | 8 |
| ATOM | 6494 | N | LEU | C | 382 | 55.110 | 99.174 | 3.543 | 1.00 | 43.72 | 7 |
| ATOM | 6495 | CA | LEU | C | 382 | 53.730 | 99.385 | 3.939 | 1.00 | 43.03 | 6 |
| ATOM | 6496 | CB | LEU | C | 382 | 52.910 | 99.874 | 2.755 | 1.00 | 18.57 | 6 |
| ATOM | 6497 | CG | LEU | C | 382 | 51.678 | 100.682 | 3.149 | 1.00 | 17.35 | 6 |
| ATOM | 6498 | CD1 | LEU | C | 382 | 52.011 | 101.661 | 4.281 | 1.00 | 16.89 | 6 |
| ATOM | 6499 | CD2 | LEU | C | 382 | 51.174 | 101.432 | 1.931 | 1.00 | 18.60 | 6 |
| ATOM | 6500 | C | LEU | C | 382 | 53.094 | 98.137 | 4.533 | 1.00 | 44.12 | 6 |
| ATOM | 6501 | O | LEU | C | 382 | 52.279 | 98.223 | 5.453 | 1.00 | 43.72 | 8 |
| ATOM | 6502 | N | ARG | C | 383 | 53.457 | 96.972 | 4.013 | 1.00 | 111.91 | 7 |
| ATOM | 6503 | CA | ARG | C | 383 | 52.903 | 95.743 | 4.554 | 1.00 | 113.91 | 6 |
| ATOM | 6504 | CB | ARG | C | 383 | 53.065 | 94.581 | 3.566 | 1.00 | 196.78 | 6 |
| ATOM | 6505 | CG | ARG | C | 383 | 52.286 | 93.329 | 3.965 | 1.00 | 200.48 | 6 |
| ATOM | 6506 | CD | ARG | C | 383 | 52.355 | 92.239 | 2.902 | 1.00 | 203.04 | 6 |
| ATOM | 6507 | NE | ARG | C | 383 | 51.615 | 91.043 | 3.305 | 1.00 | 203.93 | 7 |
| ATOM | 6508 | CZ | ARG | C | 383 | 51.507 | 89.943 | 2.565 | 1.00 | 204.05 | 6 |
| ATOM | 6509 | NH1 | ARG | C | 383 | 52.091 | 89.879 | 1.374 | 1.00 | 205.14 | 7 |
| ATOM | 6510 | NH2 | ARG | C | 383 | 50.816 | 88.904 | 3.015 | 1.00 | 203.29 | 7 |
| ATOM | 6511 | C | ARG | C | 383 | 53.667 | 95.462 | 5.840 | 1.00 | 113.54 | 6 |
| ATOM | 6512 | O | ARG | C | 383 | 53.136 | 94.859 | 6.774 | 1.00 | 114.76 | 8 |
| ATOM | 6513 | N | GLU | C | 384 | 54.914 | 95.924 | 5.882 | 1.00 | 34.91 | 7 |
| ATOM | 6514 | CA | GLU | C | 384 | 55.758 | 95.738 | 7.052 | 1.00 | 32.88 | 6 |
| ATOM | 6515 | CB | GLU | C | 384 | 57.159 | 96.280 | 6.797 | 1.00 | 64.39 | 6 |
| ATOM | 6516 | CG | GLU | C | 384 | 58.111 | 96.040 | 7.938 | 1.00 | 65.02 | 6 |
| ATOM | 6517 | CD | GLU | C | 384 | 59.470 | 95.580 | 7.467 | 1.00 | 65.71 | 6 |
| ATOM | 6518 | OE1 | GLU | C | 384 | 60.072 | 96.266 | 6.612 | 1.00 | 66.11 | 8 |
| ATOM | 6519 | OE2 | GLU | C | 384 | 59.936 | 94.529 | 7.956 | 1.00 | 66.58 | 8 |
| ATOM | 6520 | C | GLU | C | 384 | 55.133 | 96.458 | 8.230 | 1.00 | 31.68 | 6 |
| ATOM | 6521 | O | GLU | C | 384 | 55.093 | 95.928 | 9.337 | 1.00 | 32.24 | 8 |
| ATOM | 6522 | N | PHE | C | 385 | 54.636 | 97.669 | 7.996 | 1.00 | 42.85 | 7 |
| ATOM | 6523 | CA | PHE | C | 385 | 53.993 | 98.427 | 9.067 | 1.00 | 42.02 | 6 |
| ATOM | 6524 | CB | PHE | C | 385 | 53.570 | 99.817 | 8.589 | 1.00 | 39.62 | 6 |
| ATOM | 6525 | CG | PHE | C | 385 | 52.555 | 100.480 | 9.484 | 1.00 | 39.09 | 6 |
| ATOM | 6526 | CD1 | PHE | C | 385 | 52.914 | 100.936 | 10.746 | 1.00 | 38.59 | 6 |
| ATOM | 6527 | CD2 | PHE | C | 385 | 51.236 | 100.607 | 9.082 | 1.00 | 39.56 | 6 |
| ATOM | 6528 | CE1 | PHE | C | 385 | 51.977 | 101.504 | 11.594 | 1.00 | 37.75 | 6 |
| ATOM | 6529 | CE2 | PHE | C | 385 | 50.293 | 101.171 | 9.922 | 1.00 | 39.49 | 6 |
| ATOM | 6530 | CZ | PHE | C | 385 | 50.662 | 101.620 | 11.179 | 1.00 | 38.51 | 6 |
| ATOM | 6531 | C | PHE | C | 385 | 52.756 | 97.691 | 9.555 | 1.00 | 40.74 | 6 |
| ATOM | 6532 | O | PHE | C | 385 | 52.773 | 97.085 | 10.621 | 1.00 | 40.68 | 8 |
| ATOM | 6533 | N | PHE | C | 386 | 51.688 | 97.740 | 8.765 | 1.00 | 28.00 | 7 |
| ATOM | 6534 | CA | PHE | C | 386 | 50.450 | 97.090 | 9.142 | 1.00 | 27.86 | 6 |
| ATOM | 6535 | CB | PHE | C | 386 | 49.583 | 96.816 | 7.916 | 1.00 | 48.14 | 6 |
| ATOM | 6536 | CG | PHE | C | 386 | 48.604 | 97.905 | 7.641 | 1.00 | 47.19 | 6 |
| ATOM | 6537 | CD1 | PHE | C | 386 | 48.912 | 98.915 | 6.739 | 1.00 | 46.13 | 6 |
| ATOM | 6538 | CD2 | PHE | C | 386 | 47.418 | 97.989 | 8.375 | 1.00 | 46.56 | 6 |
| ATOM | 6539 | CE1 | PHE | C | 386 | 48.060 | 100.004 | 6.570 | 1.00 | 44.96 | 6 |
| ATOM | 6540 | CE2 | PHE | C | 386 | 46.557 | 99.075 | 8.217 | 1.00 | 44.71 | 6 |
| ATOM | 6541 | CZ | PHE | C | 386 | 46.880 | 100.085 | 7.314 | 1.00 | 44.87 | 6 |
| ATOM | 6542 | C | PHE | C | 386 | 50.594 | 95.824 | 9.966 | 1.00 | 28.13 | 6 |
| ATOM | 6543 | O | PHE | C | 386 | 49.966 | 95.712 | 11.009 | 1.00 | 27.05 | 8 |
| ATOM | 6544 | N | SER | C | 387 | 51.401 | 94.862 | 9.533 | 1.00 | 26.30 | 7 |
| ATOM | 6545 | CA | SER | C | 387 | 51.539 | 93.658 | 10.342 | 1.00 | 26.99 | 6 |
| ATOM | 6546 | CB | SER | C | 387 | 51.007 | 92.444 | 9.588 | 1.00 | 123.05 | 6 |
| ATOM | 6547 | OG | SER | C | 387 | 51.207 | 91.255 | 10.333 | 1.00 | 126.81 | 8 |
| ATOM | 6548 | C | SER | C | 387 | 52.967 | 93.399 | 10.753 | 1.00 | 26.19 | 6 |
| ATOM | 6549 | O | SER | C | 387 | 53.561 | 92.448 | 10.290 | 1.00 | 25.05 | 8 |
| ATOM | 6550 | N | ARG | C | 388 | 53.502 | 94.244 | 11.625 | 1.00 | 37.99 | 7 |
| ATOM | 6551 | CA | ARG | C | 388 | 54.870 | 94.131 | 12.114 | 1.00 | 38.08 | 6 |
| ATOM | 6552 | CB | ARG | C | 388 | 55.883 | 94.089 | 10.960 | 1.00 | 112.87 | 6 |
| ATOM | 6553 | CG | ARG | C | 388 | 56.209 | 92.692 | 10.435 | 1.00 | 116.98 | 6 |
| ATOM | 6554 | CD | ARG | C | 388 | 56.968 | 92.739 | 9.115 | 1.00 | 119.48 | 6 |
| ATOM | 6555 | NE | ARG | C | 388 | 57.168 | 91.404 | 8.555 | 1.00 | 121.55 | 7 |
| ATOM | 6556 | CZ | ARG | C | 388 | 57.689 | 91.169 | 7.355 | 1.00 | 122.29 | 6 |
| ATOM | 6557 | NH1 | ARG | C | 388 | 58.063 | 92.179 | 6.585 | 1.00 | 121.93 | 7 |
| ATOM | 6558 | NH2 | ARG | C | 388 | 57.838 | 89.925 | 6.925 | 1.00 | 123.26 | 7 |
| ATOM | 6559 | C | ARG | C | 388 | 55.160 | 95.331 | 12.992 | 1.00 | 35.86 | 6 |
| ATOM | 6560 | O | ARG | C | 388 | 56.278 | 95.533 | 13.454 | 1.00 | 36.12 | 8 |
| ATOM | 6561 | N | SER | C | 389 | 54.144 | 96.150 | 13.197 | 1.00 | 45.39 | 7 |
| ATOM | 6562 | CA | SER | C | 389 | 54.284 | 97.320 | 14.046 | 1.00 | 43.00 | 6 |
| ATOM | 6563 | CB | SER | C | 389 | 53.325 | 98.432 | 13.611 | 1.00 | 39.12 | 6 |
| ATOM | 6564 | OG | SER | C | 389 | 53.341 | 99.510 | 14.528 | 1.00 | 35.94 | 8 |
| ATOM | 6565 | C | SER | C | 389 | 53.889 | 96.839 | 15.419 | 1.00 | 42.79 | 6 |
| ATOM | 6566 | O | SER | C | 389 | 53.447 | 95.696 | 15.573 | 1.00 | 42.20 | 8 |
| ATOM | 6567 | N | GLN | C | 390 | 54.084 | 97.693 | 16.419 | 1.00 | 43.33 | 7 |
| ATOM | 6568 | CA | GLN | C | 390 | 53.683 | 97.352 | 17.770 | 1.00 | 41.60 | 6 |
| ATOM | 6569 | CB | GLN | C | 390 | 54.782 | 97.640 | 18.811 | 1.00 | 54.20 | 6 |
| ATOM | 6570 | CG | GLN | C | 390 | 55.759 | 98.776 | 18.515 | 1.00 | 55.42 | 6 |
| ATOM | 6571 | CD | GLN | C | 390 | 56.733 | 99.036 | 19.681 | 1.00 | 57.75 | 6 |
| ATOM | 6572 | OE1 | GLN | C | 390 | 57.351 | 98.112 | 20.216 | 1.00 | 57.83 | 8 |
| ATOM | 6573 | NE2 | GLN | C | 390 | 56.871 | 100.304 | 20.066 | 1.00 | 59.66 | 7 |
| ATOM | 6574 | C | GLN | C | 390 | 52.462 | 98.195 | 18.012 | 1.00 | 39.24 | 6 |
| ATOM | 6575 | O | GLN | C | 390 | 51.910 | 98.210 | 19.096 | 1.00 | 39.78 | 8 |
| ATOM | 6576 | N | LEU | C | 391 | 52.048 | 98.904 | 16.973 | 1.00 | 22.08 | 7 |
| ATOM | 6577 | CA | LEU | C | 391 | 50.861 | 99.723 | 17.069 | 1.00 | 21.29 | 6 |
| ATOM | 6578 | CB | LEU | C | 391 | 51.148 | 101.173 | 16.652 | 1.00 | 15.77 | 6 |
| ATOM | 6579 | CG | LEU | C | 391 | 50.698 | 102.369 | 17.508 | 1.00 | 13.87 | 6 |
| ATOM | 6580 | CD1 | LEU | C | 391 | 51.031 | 102.192 | 18.960 | 1.00 | 13.87 | 6 |
| ATOM | 6581 | CD2 | LEU | C | 391 | 51.411 | 103.590 | 17.014 | 1.00 | 13.87 | 6 |
| ATOM | 6582 | C | LEU | C | 391 | 49.820 | 99.089 | 16.193 | 1.00 | 22.68 | 6 |
| ATOM | 6583 | O | LEU | C | 391 | 48.774 | 99.678 | 15.942 | 1.00 | 22.06 | 8 |
| ATOM | 6584 | N | SER | C | 392 | 50.139 | 97.909 | 15.671 | 1.00 | 43.95 | 7 |
| ATOM | 6585 | CA | SER | C | 392 | 49.164 | 97.169 | 14.890 | 1.00 | 46.21 | 6 |
| ATOM | 6586 | CB | SER | C | 392 | 49.715 | 96.792 | 13.557 | 1.00 | 85.53 | 6 |
| ATOM | 6587 | OG | SER | C | 392 | 48.836 | 95.907 | 12.903 | 1.00 | 90.30 | 8 |
| ATOM | 6588 | C | SER | C | 392 | 48.967 | 95.890 | 15.691 | 1.00 | 46.84 | 6 |
| ATOM | 6589 | O | SER | C | 392 | 49.482 | 94.831 | 15.306 | 1.00 | 45.92 | 8 |
| ATOM | 6590 | N | GLN | C | 393 | 48.147 | 95.990 | 16.740 | 1.00 | 47.13 | 7 |
| ATOM | 6591 | CA | GLN | C | 393 | 47.832 | 94.925 | 17.667 | 1.00 | 47.20 | 6 |
| ATOM | 6592 | CB | GLN | C | 393 | 47.217 | 95.540 | 18.898 | 1.00 | 30.87 | 6 |
| ATOM | 6593 | CG | GLN | C | 393 | 47.925 | 96.720 | 19.395 | 1.00 | 28.44 | 6 |
| ATOM | 6594 | CD | GLN | C | 393 | 47.360 | 97.211 | 20.675 | 1.00 | 25.75 | 6 |
| ATOM | 6595 | OE1 | GLN | C | 393 | 46.354 | 96.725 | 21.166 | 1.00 | 24.51 | 8 |
| ATOM | 6596 | NE2 | GLN | C | 393 | 48.006 | 98.187 | 21.235 | 1.00 | 23.12 | 7 |
| ATOM | 6597 | C | GLN | C | 393 | 46.929 | 93.850 | 17.118 | 1.00 | 47.76 | 6 |
| ATOM | 6598 | O | GLN | C | 393 | 46.520 | 93.884 | 15.954 | 1.00 | 47.04 | 8 |
| ATOM | 6599 | N | PHE | C | 394 | 46.632 | 92.891 | 17.987 | 1.00 | 100.33 | 7 |
| ATOM | 6600 | CA | PHE | C | 394 | 45.819 | 91.735 | 17.611 | 1.00 | 101.57 | 6 |
| ATOM | 6601 | CB | PHE | C | 394 | 46.088 | 90.558 | 18.496 | 1.00 | 55.89 | 6 |
| ATOM | 6602 | CG | PHE | C | 394 | 45.937 | 89.199 | 17.814 | 1.00 | 56.43 | 6 |
| ATOM | 6603 | CD1 | PHE | C | 394 | 47.022 | 88.610 | 17.163 | 1.00 | 56.75 | 6 |
| ATOM | 6604 | CD2 | PHE | C | 394 | 44.729 | 88.501 | 17.847 | 1.00 | 56.42 | 6 |
| ATOM | 6605 | CE1 | PHE | C | 394 | 46.903 | 87.378 | 16.571 | 1.00 | 55.89 | 6 |
| ATOM | 6606 | CE2 | PHE | C | 394 | 44.614 | 87.263 | 17.251 | 1.00 | 56.09 | 6 |
| ATOM | 6607 | CZ | PHE | C | 394 | 45.701 | 86.706 | 16.613 | 1.00 | 56.21 | 6 |
| ATOM | 6608 | C | PHE | C | 394 | 44.386 | 92.136 | 17.908 | 1.00 | 102.12 | 6 |
| ATOM | 6609 | O | PHE | C | 394 | 44.148 | 93.111 | 18.590 | 1.00 | 105.06 | 8 |
| ATOM | 6610 | N | LYS | C | 395 | 43.398 | 91.433 | 17.396 | 1.00 | 43.81 | 7 |
| ATOM | 6611 | CA | LYS | C | 395 | 42.047 | 91.882 | 17.648 | 1.00 | 42.16 | 6 |
| ATOM | 6612 | CB | LYS | C | 395 | 41.163 | 91.329 | 16.587 | 1.00 | 29.19 | 6 |
| ATOM | 6613 | CG | LYS | C | 395 | 39.781 | 91.752 | 16.820 | 1.00 | 28.08 | 6 |
| ATOM | 6614 | CD | LYS | C | 395 | 39.421 | 92.968 | 15.985 | 1.00 | 27.79 | 6 |
| ATOM | 6615 | CE | LYS | C | 395 | 37.907 | 93.165 | 15.857 | 1.00 | 28.90 | 6 |
| ATOM | 6616 | NZ | LYS | C | 395 | 37.246 | 92.603 | 14.623 | 1.00 | 30.18 | 7 |
| ATOM | 6617 | C | LYS | C | 395 | 41.661 | 91.354 | 18.981 | 1.00 | 42.33 | 6 |
| ATOM | 6618 | O | LYS | C | 395 | 41.706 | 90.197 | 19.193 | 1.00 | 44.26 | 8 |
| ATOM | 6619 | N | ASP | C | 396 | 41.363 | 92.120 | 19.918 | 1.00 | 50.17 | 7 |
| ATOM | 6620 | CA | ASP | C | 396 | 40.920 | 91.730 | 21.255 | 1.00 | 49.83 | 6 |
| ATOM | 6621 | CB | ASP | C | 396 | 40.968 | 92.942 | 22.177 | 1.00 | 62.41 | 6 |
| ATOM | 6622 | CG | ASP | C | 396 | 40.953 | 92.600 | 23.650 | 1.00 | 63.51 | 6 |
| ATOM | 6623 | OD1 | ASP | C | 396 | 39.859 | 92.509 | 24.213 | 1.00 | 64.56 | 8 |
| ATOM | 6624 | OD2 | ASP | C | 396 | 42.030 | 92.530 | 24.284 | 1.00 | 62.94 | 8 |
| ATOM | 6625 | C | ASP | C | 396 | 39.518 | 91.320 | 20.926 | 1.00 | 49.88 | 6 |
| ATOM | 6626 | O | ASP | C | 396 | 38.765 | 92.120 | 20.429 | 1.00 | 50.51 | 8 |
| ATOM | 6627 | N | GLU | C | 397 | 39.080 | 90.123 | 21.021 | 1.00 | 56.43 | 7 |
| ATOM | 6628 | CA | GLU | C | 397 | 37.715 | 89.814 | 20.676 | 1.00 | 55.07 | 6 |
| ATOM | 6629 | CB | GLU | C | 397 | 37.666 | 89.323 | 19.251 | 1.00 | 49.17 | 6 |
| ATOM | 6630 | CG | GLU | C | 397 | 37.220 | 90.347 | 18.256 | 1.00 | 54.30 | 6 |
| ATOM | 6631 | CD | GLU | C | 397 | 37.118 | 89.754 | 16.863 | 1.00 | 58.33 | 6 |
| ATOM | 6632 | OE1 | GLU | C | 397 | 36.404 | 90.376 | 16.030 | 1.00 | 60.41 | 8 |
| ATOM | 6633 | OE2 | GLU | C | 397 | 37.749 | 88.677 | 16.650 | 1.00 | 60.82 | 8 |
| ATOM | 6634 | C | GLU | C | 397 | 37.006 | 88.814 | 21.516 | 1.00 | 52.66 | 6 |
| ATOM | 6635 | O | GLU | C | 397 | 36.171 | 88.083 | 20.991 | 1.00 | 53.22 | 8 |
| ATOM | 6636 | N | THR | C | 398 | 37.320 | 88.765 | 22.800 | 1.00 | 28.42 | 7 |
| ATOM | 6637 | CA | THR | C | 398 | 36.665 | 87.811 | 23.652 | 1.00 | 25.27 | 6 |
| ATOM | 6638 | CB | THR | C | 398 | 37.247 | 87.863 | 25.038 | 1.00 | 19.82 | 6 |
| ATOM | 6639 | OG1 | THR | C | 398 | 38.607 | 88.268 | 24.933 | 1.00 | 19.57 | 8 |
| ATOM | 6640 | CG2 | THR | C | 398 | 37.222 | 86.489 | 25.695 | 1.00 | 18.63 | 6 |
| ATOM | 6641 | C | THR | C | 398 | 35.177 | 88.132 | 23.682 | 1.00 | 25.57 | 6 |
| ATOM | 6642 | O | THR | C | 398 | 34.360 | 87.249 | 23.430 | 1.00 | 26.94 | 8 |
| ATOM | 6643 | N | ASN | C | 399 | 34.811 | 89.379 | 23.988 | 1.00 | 37.48 | 7 |
| ATOM | 6644 | CA | ASN | C | 399 | 33.392 | 89.759 | 23.996 | 1.00 | 35.58 | 6 |
| ATOM | 6645 | CB | ASN | C | 399 | 32.902 | 90.052 | 25.448 | 1.00 | 21.35 | 6 |
| ATOM | 6646 | CG | ASN | C | 399 | 32.983 | 91.536 | 25.855 | 1.00 | 19.01 | 6 |
| ATOM | 6647 | OD1 | ASN | C | 399 | 32.951 | 91.854 | 27.043 | 1.00 | 17.99 | 8 |
| ATOM | 6648 | ND2 | ASN | C | 399 | 33.059 | 92.437 | 24.884 | 1.00 | 17.39 | 7 |
| ATOM | 6649 | C | ASN | C | 399 | 33.212 | 90.945 | 23.040 | 1.00 | 35.20 | 6 |
| ATOM | 6650 | O | ASN | C | 399 | 34.146 | 91.715 | 22.825 | 1.00 | 35.32 | 8 |
| ATOM | 6651 | N | PRO | C | 400 | 32.015 | 91.103 | 22.442 | 1.00 | 48.40 | 7 |
| ATOM | 6652 | CD | PRO | C | 400 | 30.737 | 90.377 | 22.567 | 1.00 | 16.55 | 6 |
| ATOM | 6653 | CA | PRO | C | 400 | 31.877 | 92.238 | 21.531 | 1.00 | 47.22 | 6 |
| ATOM | 6654 | CB | PRO | C | 400 | 30.374 | 92.226 | 21.168 | 1.00 | 14.25 | 6 |
| ATOM | 6655 | CG | PRO | C | 400 | 29.724 | 91.456 | 22.247 | 1.00 | 13.87 | 6 |
| ATOM | 6656 | C | PRO | C | 400 | 32.393 | 93.590 | 22.040 | 1.00 | 46.64 | 6 |
| ATOM | 6657 | O | PRO | C | 400 | 33.057 | 94.295 | 21.290 | 1.00 | 46.70 | 8 |
| ATOM | 6658 | N | LEU | C | 401 | 32.126 | 93.962 | 23.290 | 1.00 | 28.24 | 7 |
| ATOM | 6659 | CA | LEU | C | 401 | 32.618 | 95.259 | 23.764 | 1.00 | 27.85 | 6 |
| ATOM | 6660 | CB | LEU | C | 401 | 32.372 | 95.455 | 25.262 | 1.00 | 13.87 | 6 |
| ATOM | 6661 | CG | LEU | C | 401 | 33.105 | 96.654 | 25.889 | 1.00 | 13.87 | 6 |
| ATOM | 6662 | CD1 | LEU | C | 401 | 32.619 | 97.953 | 25.329 | 1.00 | 13.87 | 6 |
| ATOM | 6663 | CD2 | LEU | C | 401 | 32.886 | 96.633 | 27.359 | 1.00 | 13.87 | 6 |
| ATOM | 6664 | C | LEU | C | 401 | 34.109 | 95.417 | 23.486 | 1.00 | 28.34 | 6 |
| ATOM | 6665 | O | LEU | C | 401 | 34.541 | 96.405 | 22.876 | 1.00 | 28.00 | 8 |
| ATOM | 6666 | N | SER | C | 402 | 34.879 | 94.434 | 23.946 | 1.00 | 13.95 | 7 |
| ATOM | 6667 | CA | SER | C | 402 | 36.327 | 94.383 | 23.779 | 1.00 | 15.21 | 6 |
| ATOM | 6668 | CB | SER | C | 402 | 36.784 | 92.924 | 23.812 | 1.00 | 38.02 | 6 |
| ATOM | 6669 | OG | SER | C | 402 | 36.100 | 92.173 | 24.805 | 1.00 | 39.19 | 8 |
| ATOM | 6670 | C | SER | C | 402 | 36.778 | 95.020 | 22.465 | 1.00 | 16.19 | 6 |
| ATOM | 6671 | O | SER | C | 402 | 37.133 | 96.182 | 22.439 | 1.00 | 16.43 | 8 |
| ATOM | 6672 | N | SER | C | 403 | 36.760 | 94.251 | 21.380 | 1.00 | 15.62 | 7 |
| ATOM | 6673 | CA | SER | C | 403 | 37.176 | 94.747 | 20.079 | 1.00 | 17.34 | 6 |
| ATOM | 6674 | CB | SER | C | 403 | 36.604 | 93.877 | 18.963 | 1.00 | 62.46 | 6 |
| ATOM | 6675 | OG | SER | C | 403 | 35.382 | 94.419 | 18.498 | 1.00 | 67.82 | 8 |
| ATOM | 6676 | C | SER | C | 403 | 36.755 | 96.199 | 19.860 | 1.00 | 17.00 | 6 |
| ATOM | 6677 | O | SER | C | 403 | 37.539 | 97.002 | 19.346 | 1.00 | 15.83 | 8 |
| ATOM | 6678 | N | LEU | C | 404 | 35.521 | 96.540 | 20.234 | 1.00 | 27.97 | 7 |
| ATOM | 6679 | CA | LEU | C | 404 | 35.026 | 97.916 | 20.092 | 1.00 | 28.89 | 6 |
| ATOM | 6680 | CB | LEU | C | 404 | 33.603 | 98.034 | 20.672 | 1.00 | 19.74 | 6 |
| ATOM | 6681 | CG | LEU | C | 404 | 32.946 | 99.424 | 20.794 | 1.00 | 19.85 | 6 |
| ATOM | 6682 | CD1 | LEU | C | 404 | 33.091 | 100.242 | 19.523 | 1.00 | 19.19 | 6 |
| ATOM | 6683 | CD2 | LEU | C | 404 | 31.480 | 99.249 | 21.115 | 1.00 | 19.89 | 6 |
| ATOM | 6684 | C | LEU | C | 404 | 36.000 | 98.822 | 20.852 | 1.00 | 29.54 | 6 |
| ATOM | 6685 | O | LEU | C | 404 | 36.624 | 99.719 | 20.285 | 1.00 | 28.10 | 8 |
| ATOM | 6686 | N | ARG | C | 405 | 36.136 | 98.552 | 22.141 | 1.00 | 35.76 | 7 |
| ATOM | 6687 | CA | ARG | C | 405 | 37.039 | 99.292 | 23.000 | 1.00 | 38.91 | 6 |
| ATOM | 6688 | CB | ARG | C | 405 | 37.145 | 98.572 | 24.342 | 1.00 | 66.63 | 6 |
| ATOM | 6689 | CG | ARG | C | 405 | 38.233 | 99.090 | 25.252 | 1.00 | 71.12 | 6 |
| ATOM | 6690 | CD | ARG | C | 405 | 38.340 | 98.252 | 26.518 | 1.00 | 73.97 | 6 |
| ATOM | 6691 | NE | ARG | C | 405 | 39.437 | 98.713 | 27.360 | 1.00 | 76.85 | 7 |
| ATOM | 6692 | CZ | ARG | C | 405 | 39.900 | 98.054 | 28.416 | 1.00 | 78.23 | 6 |
| ATOM | 6693 | NH1 | ARG | C | 405 | 39.362 | 96.894 | 28.771 | 1.00 | 78.51 | 7 |
| ATOM | 6694 | NH2 | ARG | C | 405 | 40.913 | 98.555 | 29.111 | 1.00 | 77.42 | 7 |
| ATOM | 6695 | C | ARG | C | 405 | 38.427 | 99.411 | 22.381 | 1.00 | 40.01 | 6 |
| ATOM | 6696 | O | ARG | C | 405 | 39.073 | 100.452 | 22.447 | 1.00 | 40.60 | 8 |
| ATOM | 6697 | N | HIS | C | 406 | 38.886 | 98.338 | 21.761 | 1.00 | 71.68 | 7 |
| ATOM | 6698 | CA | HIS | C | 406 | 40.214 | 98.343 | 21.181 | 1.00 | 71.27 | 6 |
| ATOM | 6699 | CB | HIS | C | 406 | 40.476 | 97.032 | 20.471 | 1.00 | 45.63 | 6 |
| ATOM | 6700 | CG | HIS | C | 406 | 41.925 | 96.763 | 20.246 | 1.00 | 45.60 | 6 |
| ATOM | 6701 | CD2 | HIS | C | 406 | 42.673 | 95.666 | 20.502 | 1.00 | 45.37 | 6 |
| ATOM | 6702 | ND1 | HIS | C | 406 | 42.777 | 97.691 | 19.694 | 1.00 | 45.64 | 7 |
| ATOM | 6703 | CE1 | HIS | C | 406 | 43.990 | 97.177 | 19.620 | 1.00 | 46.13 | 6 |
| ATOM | 6704 | NE2 | HIS | C | 406 | 43.954 | 95.949 | 20.104 | 1.00 | 45.14 | 7 |
| ATOM | 6705 | C | HIS | C | 406 | 40.507 | 99.480 | 20.220 | 1.00 | 70.89 | 6 |
| ATOM | 6706 | O | HIS | C | 406 | 41.665 | 99.834 | 20.013 | 1.00 | 71.72 | 8 |
| ATOM | 6707 | N | LYS | C | 407 | 39.474 | 100.052 | 19.621 | 1.00 | 32.66 | 7 |
| ATOM | 6708 | CA | LYS | C | 407 | 39.699 | 101.145 | 18.682 | 1.00 | 32.24 | 6 |
| ATOM | 6709 | CB | LYS | C | 407 | 38.658 | 101.092 | 17.572 | 1.00 | 49.47 | 6 |
| ATOM | 6710 | CG | LYS | C | 407 | 37.803 | 99.846 | 17.643 | 1.00 | 50.89 | 6 |
| ATOM | 6711 | CD | LYS | C | 407 | 37.825 | 99.073 | 16.360 | 1.00 | 51.05 | 6 |
| ATOM | 6712 | CE | LYS | C | 407 | 36.856 | 97.930 | 16.448 | 1.00 | 50.82 | 6 |
| ATOM | 6713 | NZ | LYS | C | 407 | 36.828 | 97.249 | 15.154 | 1.00 | 54.18 | 7 |
| ATOM | 6714 | C | LYS | C | 407 | 39.613 | 102.453 | 19.435 | 1.00 | 31.38 | 6 |
| ATOM | 6715 | O | LYS | C | 407 | 40.315 | 103.415 | 19.135 | 1.00 | 30.71 | 8 |
| ATOM | 6716 | N | ARG | C | 408 | 38.742 | 102.463 | 20.430 | 1.00 | 37.35 | 7 |
| ATOM | 6717 | CA | ARG | C | 408 | 38.541 | 103.623 | 21.267 | 1.00 | 37.07 | 6 |
| ATOM | 6718 | CB | ARG | C | 408 | 37.306 | 103.398 | 22.149 | 1.00 | 46.72 | 6 |
| ATOM | 6719 | CG | ARG | C | 408 | 36.014 | 103.190 | 21.383 | 1.00 | 46.51 | 6 |
| ATOM | 6720 | CD | ARG | C | 408 | 35.442 | 104.515 | 20.974 | 1.00 | 48.61 | 6 |
| ATOM | 6721 | NE | ARG | C | 408 | 35.078 | 105.309 | 22.145 | 1.00 | 50.98 | 7 |
| ATOM | 6722 | CZ | ARG | C | 408 | 34.973 | 106.638 | 22.156 | 1.00 | 52.08 | 6 |
| ATOM | 6723 | NH1 | ARG | C | 408 | 35.204 | 107.354 | 21.058 | 1.00 | 52.11 | 7 |
| ATOM | 6724 | NH2 | ARG | C | 408 | 34.643 | 107.259 | 23.273 | 1.00 | 51.65 | 7 |
| ATOM | 6725 | C | ARG | C | 408 | 39.796 | 103.807 | 22.132 | 1.00 | 36.51 | 6 |
| ATOM | 6726 | O | ARG | C | 408 | 39.803 | 104.612 | 23.066 | 1.00 | 36.16 | 8 |
| ATOM | 6727 | N | ARG | C | 409 | 40.862 | 103.070 | 21.813 | 1.00 | 35.92 | 7 |
| ATOM | 6728 | CA | ARG | C | 409 | 42.100 | 103.162 | 22.593 | 1.00 | 35.68 | 6 |
| ATOM | 6729 | CB | ARG | C | 409 | 42.737 | 101.794 | 22.782 | 1.00 | 37.02 | 6 |
| ATOM | 6730 | CG | ARG | C | 409 | 44.122 | 101.843 | 23.419 | 1.00 | 35.78 | 6 |
| ATOM | 6731 | CD | ARG | C | 409 | 44.655 | 100.438 | 23.564 | 1.00 | 36.76 | 6 |
| ATOM | 6732 | NE | ARG | C | 409 | 43.710 | 99.638 | 24.333 | 1.00 | 38.64 | 7 |
| ATOM | 6733 | CZ | ARG | C | 409 | 43.783 | 98.323 | 24.497 | 1.00 | 39.22 | 6 |
| ATOM | 6734 | NH1 | ARG | C | 409 | 44.767 | 97.629 | 23.948 | 1.00 | 39.62 | 7 |
| ATOM | 6735 | NH2 | ARG | C | 409 | 42.857 | 97.701 | 25.208 | 1.00 | 41.22 | 7 |
| ATOM | 6736 | C | ARG | C | 409 | 43.178 | 104.088 | 22.086 | 1.00 | 35.88 | 6 |
| ATOM | 6737 | O | ARG | C | 409 | 43.510 | 104.107 | 20.896 | 1.00 | 35.91 | 8 |
| ATOM | 6738 | N | ILE | C | 410 | 43.729 | 104.842 | 23.030 | 1.00 | 35.02 | 7 |
| ATOM | 6739 | CA | ILE | C | 410 | 44.799 | 105.785 | 22.765 | 1.00 | 36.04 | 6 |
| ATOM | 6740 | CB | ILE | C | 410 | 44.591 | 107.153 | 23.528 | 1.00 | 32.78 | 6 |
| ATOM | 6741 | CG2 | ILE | C | 410 | 45.939 | 107.795 | 23.826 | 1.00 | 34.25 | 6 |
| ATOM | 6742 | CG1 | ILE | C | 410 | 43.759 | 108.137 | 22.696 | 1.00 | 31.74 | 6 |
| ATOM | 6743 | CD1 | ILE | C | 410 | 42.278 | 107.862 | 22.649 | 1.00 | 30.33 | 6 |
| ATOM | 6744 | C | ILE | C | 410 | 46.034 | 105.089 | 23.318 | 1.00 | 37.94 | 6 |
| ATOM | 6745 | O | ILE | C | 410 | 46.056 | 104.717 | 24.493 | 1.00 | 38.10 | 8 |
| ATOM | 6746 | N | SER | C | 411 | 47.038 | 104.885 | 22.471 | 1.00 | 24.18 | 7 |
| ATOM | 6747 | CA | SER | C | 411 | 48.268 | 104.250 | 22.909 | 1.00 | 27.82 | 6 |
| ATOM | 6748 | CB | SER | C | 411 | 48.783 | 103.266 | 21.861 | 1.00 | 157.88 | 6 |
| ATOM | 6749 | OG | SER | C | 411 | 48.934 | 101.957 | 22.435 | 1.00 | 162.92 | 8 |
| ATOM | 6750 | C | SER | C | 411 | 49.328 | 105.306 | 23.163 | 1.00 | 28.22 | 6 |
| ATOM | 6751 | O | SER | C | 411 | 49.157 | 106.472 | 22.810 | 1.00 | 27.60 | 8 |
| ATOM | 6752 | N | ALA | C | 412 | 50.424 | 104.904 | 23.783 | 1.00 | 55.73 | 7 |
| ATOM | 6753 | CA | ALA | C | 412 | 51.484 | 105.844 | 24.063 | 1.00 | 58.06 | 6 |
| ATOM | 6754 | CB | ALA | C | 412 | 51.304 | 106.428 | 25.444 | 1.00 | 70.88 | 6 |
| ATOM | 6755 | C | ALA | C | 412 | 52.792 | 105.094 | 23.969 | 1.00 | 59.54 | 6 |
| ATOM | 6756 | O | ALA | C | 412 | 53.835 | 105.674 | 23.677 | 1.00 | 61.33 | 8 |
| ATOM | 6757 | N | LEU | C | 413 | 52.763 | 103.845 | 23.911 | 1.00 | 56.28 | 7 |
| ATOM | 6758 | CA | LEU | C | 413 | 53.917 | 102.970 | 24.143 | 1.00 | 56.99 | 6 |
| ATOM | 6759 | CB | LEU | C | 413 | 53.486 | 101.686 | 24.859 | 1.00 | 33.97 | 6 |
| ATOM | 6760 | CG | LEU | C | 413 | 53.462 | 101.732 | 26.389 | 1.00 | 33.75 | 6 |
| ATOM | 6761 | CD1 | LEU | C | 413 | 53.010 | 100.396 | 26.960 | 1.00 | 34.03 | 6 |
| ATOM | 6762 | CD2 | LEU | C | 413 | 54.829 | 102.111 | 26.937 | 1.00 | 33.01 | 6 |
| ATOM | 6763 | C | LEU | C | 413 | 54.633 | 102.619 | 22.836 | 1.00 | 58.26 | 6 |
| ATOM | 6764 | O | LEU | C | 413 | 55.800 | 102.271 | 22.908 | 1.00 | 57.86 | 8 |
| ATOM | 6765 | N | GLY | C | 414 | 54.461 | 102.472 | 21.897 | 1.00 | 55.40 | 7 |
| ATOM | 6766 | CA | GLY | C | 414 | 54.429 | 102.388 | 20.433 | 1.00 | 58.04 | 6 |
| ATOM | 6767 | C | GLY | C | 414 | 55.480 | 103.322 | 19.847 | 1.00 | 60.19 | 6 |
| ATOM | 6768 | O | GLY | C | 414 | 55.907 | 104.249 | 20.529 | 1.00 | 59.87 | 8 |
| ATOM | 6769 | N | PRO | C | 415 | 55.778 | 103.592 | 18.679 | 1.00 | 67.48 | 7 |
| ATOM | 6770 | CD | PRO | C | 415 | 54.565 | 103.100 | 17.851 | 1.00 | 42.19 | 6 |
| ATOM | 6771 | CA | PRO | C | 415 | 56.957 | 103.776 | 17.822 | 1.00 | 69.41 | 6 |
| ATOM | 6772 | CB | PRO | C | 415 | 56.489 | 103.238 | 16.456 | 1.00 | 43.88 | 6 |
| ATOM | 6773 | CG | PRO | C | 415 | 55.322 | 102.382 | 16.781 | 1.00 | 42.95 | 6 |
| ATOM | 6774 | C | PRO | C | 415 | 57.383 | 105.244 | 17.719 | 1.00 | 71.76 | 6 |
| ATOM | 6775 | O | PRO | C | 415 | 58.644 | 105.425 | 17.279 | 1.00 | 72.81 | 8 |
| ATOM | 6776 | N | GLY | C | 416 | 57.069 | 106.150 | 17.236 | 1.00 | 73.40 | 7 |
| ATOM | 6777 | CA | GLY | C | 416 | 56.907 | 107.595 | 17.251 | 1.00 | 76.08 | 6 |
| ATOM | 6778 | C | GLY | C | 416 | 58.056 | 108.227 | 18.015 | 1.00 | 78.75 | 6 |
| ATOM | 6779 | O | GLY | C | 416 | 58.600 | 107.574 | 18.941 | 1.00 | 79.56 | 8 |
| ATOM | 6780 | N | GLY | C | 417 | 58.491 | 109.387 | 17.553 | 1.00 | 208.87 | 7 |
| ATOM | 6781 | CA | GLY | C | 417 | 59.641 | 110.073 | 18.153 | 1.00 | 208.87 | 6 |
| ATOM | 6782 | C | GLY | C | 417 | 59.492 | 110.309 | 19.660 | 1.00 | 208.87 | 6 |
| ATOM | 6783 | O | GLY | C | 417 | 58.525 | 110.979 | 20.105 | 1.00 | 208.87 | 8 |
| ATOM | 6784 | N | LEU | C | 418 | 60.613 | 109.829 | 20.407 | 1.00 | 178.48 | 7 |
| ATOM | 6785 | CA | LEU | C | 418 | 60.715 | 110.086 | 21.845 | 1.00 | 178.98 | 6 |
| ATOM | 6786 | CB | LEU | C | 418 | 60.914 | 111.587 | 22.111 | 1.00 | 115.94 | 6 |
| ATOM | 6787 | CG | LEU | C | 418 | 61.291 | 112.004 | 23.540 | 1.00 | 116.07 | 6 |
| ATOM | 6788 | CD1 | LEU | C | 418 | 62.642 | 111.406 | 23.903 | 1.00 | 115.88 | 6 |
| ATOM | 6789 | CD2 | LEU | C | 418 | 61.336 | 113.523 | 23.650 | 1.00 | 115.50 | 6 |
| ATOM | 6790 | C | LEU | C | 418 | 59.450 | 109.578 | 22.554 | 1.00 | 179.32 | 6 |
| ATOM | 6791 | O | LEU | C | 418 | 59.110 | 110.018 | 23.654 | 1.00 | 179.90 | 8 |
| ATOM | 6792 | N | THR | C | 419 | 58.770 | 108.637 | 21.901 | 1.00 | 96.26 | 7 |
| ATOM | 6793 | CA | THR | C | 419 | 57.544 | 108.025 | 22.404 | 1.00 | 95.44 | 6 |
| ATOM | 6794 | CB | THR | C | 419 | 56.928 | 107.117 | 21.340 | 1.00 | 56.35 | 6 |
| ATOM | 6795 | OG1 | THR | C | 419 | 56.537 | 107.908 | 20.215 | 1.00 | 56.39 | 8 |
| ATOM | 6796 | CG2 | THR | C | 419 | 55.727 | 106.383 | 21.889 | 1.00 | 56.02 | 6 |
| ATOM | 6797 | C | THR | C | 419 | 57.804 | 107.176 | 23.635 | 1.00 | 96.03 | 6 |
| ATOM | 6798 | O | THR | C | 419 | 58.207 | 106.020 | 23.508 | 1.00 | 96.11 | 8 |
| ATOM | 6799 | N | ARG | C | 420 | 57.558 | 107.745 | 24.814 | 1.00 | 77.71 | 7 |
| ATOM | 6800 | CA | ARG | C | 420 | 57.770 | 107.055 | 26.086 | 1.00 | 77.96 | 6 |
| ATOM | 6801 | CB | ARG | C | 420 | 56.605 | 107.326 | 27.046 | 1.00 | 104.02 | 6 |
| ATOM | 6802 | CG | ARG | C | 420 | 55.915 | 108.674 | 26.853 | 1.00 | 105.07 | 6 |
| ATOM | 6803 | CD | ARG | C | 420 | 56.881 | 109.843 | 27.005 | 1.00 | 104.95 | 6 |
| ATOM | 6804 | NE | ARG | C | 420 | 56.216 | 111.139 | 26.869 | 1.00 | 105.26 | 7 |
| ATOM | 6805 | CZ | ARG | C | 420 | 56.834 | 112.314 | 26.984 | 1.00 | 106.46 | 6 |
| ATOM | 6806 | NH1 | ARG | C | 420 | 58.138 | 112.366 | 27.234 | 1.00 | 106.73 | 7 |
| ATOM | 6807 | NH2 | ARG | C | 420 | 56.146 | 113.442 | 26.857 | 1.00 | 106.90 | 7 |
| ATOM | 6808 | C | ARG | C | 420 | 57.879 | 105.554 | 25.854 | 1.00 | 78.23 | 6 |
| ATOM | 6809 | O | ARG | C | 420 | 56.919 | 104.928 | 25.390 | 1.00 | 77.77 | 8 |
| ATOM | 6810 | N | GLU | C | 421 | 59.052 | 104.981 | 26.127 | 1.00 | 66.20 | 7 |
| ATOM | 6811 | CA | GLU | C | 421 | 59.214 | 103.543 | 25.959 | 1.00 | 67.97 | 6 |
| ATOM | 6812 | CB | GLU | C | 421 | 60.691 | 103.151 | 25.841 | 1.00 | 129.07 | 6 |
| ATOM | 6813 | CG | GLU | C | 421 | 61.453 | 103.081 | 27.157 | 1.00 | 131.33 | 6 |
| ATOM | 6814 | CD | GLU | C | 421 | 62.674 | 102.170 | 27.080 | 1.00 | 133.12 | 6 |
| ATOM | 6815 | OE1 | GLU | C | 421 | 63.577 | 102.442 | 26.260 | 1.00 | 134.20 | 8 |
| ATOM | 6816 | OE2 | GLU | C | 421 | 62.731 | 101.177 | 27.842 | 1.00 | 134.22 | 8 |
| ATOM | 6817 | C | GLU | C | 421 | 58.609 | 102.964 | 27.233 | 1.00 | 68.71 | 6 |
| ATOM | 6818 | O | GLU | C | 421 | 58.182 | 103.724 | 28.104 | 1.00 | 68.46 | 8 |
| ATOM | 6819 | N | ARG | C | 422 | 58.562 | 101.641 | 27.353 | 1.00 | 76.71 | 7 |
| ATOM | 6820 | CA | ARG | C | 422 | 57.993 | 101.021 | 28.546 | 1.00 | 78.22 | 6 |
| ATOM | 6821 | CB | ARG | C | 422 | 58.513 | 99.586 | 28.707 | 1.00 | 169.99 | 6 |
| ATOM | 6822 | CG | ARG | C | 422 | 60.001 | 99.471 | 29.013 | 1.00 | 172.23 | 6 |
| ATOM | 6823 | CD | ARG | C | 422 | 60.409 | 98.015 | 29.221 | 1.00 | 174.06 | 6 |
| ATOM | 6824 | NE | ARG | C | 422 | 59.588 | 97.355 | 30.236 | 1.00 | 175.15 | 7 |
| ATOM | 6825 | CZ | ARC | C | 422 | 59.774 | 96.108 | 30.662 | 1.00 | 175.32 | 6 |
| ATOM | 6826 | NH1 | ARG | C | 422 | 60.759 | 95.373 | 30.166 | 1.00 | 175.51 | 7 |
| ATOM | 6827 | NH2 | ARC | C | 422 | 58.973 | 95.594 | 31.586 | 1.00 | 175.57 | 7 |
| ATOM | 6828 | C | ARG | C | 422 | 58.365 | 101.853 | 29.766 | 1.00 | 78.41 | 6 |
| ATOM | 6829 | O | ARG | C | 422 | 57.577 | 101.999 | 30.691 | 1.00 | 77.73 | 8 |
| ATOM | 6830 | N | ALA | C | 423 | 59.572 | 102.411 | 29.742 | 1.00 | 83.10 | 7 |
| ATOM | 6831 | CA | ALA | C | 423 | 60.073 | 103.253 | 30.823 | 1.00 | 83.57 | 6 |
| ATOM | 6832 | CB | ALA | C | 423 | 61.008 | 102.442 | 31.717 | 1.00 | 150.52 | 6 |
| ATOM | 6833 | C | ALA | C | 423 | 60.799 | 104.507 | 30.293 | 1.00 | 83.97 | 6 |
| ATOM | 6834 | O | ALA | C | 423 | 61.761 | 104.982 | 30.903 | 1.00 | 83.60 | 8 |
| ATOM | 6835 | N | GLY | C | 424 | 60.318 | 105.042 | 29.170 | 1.00 | 195.59 | 7 |
| ATOM | 6836 | CA | CLY | C | 424 | 60.923 | 106.225 | 28.569 | 1.00 | 196.13 | 6 |
| ATOM | 6837 | C | GLY | C | 424 | 61.202 | 107.343 | 29.559 | 1.00 | 196.47 | 6 |
| ATOM | 6838 | O | GLY | C | 424 | 60.606 | 107.373 | 30.636 | 1.00 | 196.41 | 8 |
| ATOM | 6839 | N | PHE | C | 425 | 62.092 | 108.271 | 29.200 | 1.00 | 134.14 | 7 |
| ATOM | 6840 | CA | PHE | C | 425 | 62.436 | 109.370 | 30.102 | 1.00 | 133.29 | 6 |
| ATOM | 6841 | CB | PHE | C | 425 | 63.622 | 110.182 | 29.559 | 1.00 | 106.82 | 6 |
| ATOM | 6842 | CG | PHE | C | 425 | 64.887 | 109.996 | 30.360 | 1.00 | 107.16 | 6 |
| ATOM | 6843 | CD1 | PHE | C | 425 | 65.712 | 108.894 | 30.144 | 1.00 | 106.92 | 6 |
| ATOM | 6844 | CD2 | PHE | C | 425 | 65.210 | 110.877 | 31.393 | 1.00 | 107.62 | 6 |
| ATOM | 6845 | CE1 | PHE | C | 425 | 66.838 | 108.665 | 30.950 | 1.00 | 107.04 | 6 |
| ATOM | 6846 | CE2 | PHE | C | 425 | 66.335 | 110.656 | 32.205 | 1.00 | 108.30 | 6 |
| ATOM | 6847 | CZ | PHE | C | 425 | 67.147 | 109.546 | 31.981 | 1.00 | 107.74 | 6 |
| ATOM | 6848 | C | PHE | C | 425 | 61.284 | 110.307 | 30.458 | 1.00 | 132.93 | 6 |
| ATOM | 6849 | O | PHE | C | 425 | 61.031 | 111.294 | 29.764 | 1.00 | 133.62 | 8 |
| ATOM | 6850 | N | ASP | C | 426 | 60.618 | 109.960 | 31.564 | 1.00 | 158.69 | 7 |
| ATOM | 6851 | CA | ASP | C | 426 | 59.477 | 110.654 | 32.177 | 1.00 | 157.54 | 6 |
| ATOM | 6852 | CB | ASP | C | 426 | 59.177 | 112.010 | 31.500 | 1.00 | 82.48 | 6 |
| ATOM | 6853 | CG | ASP | C | 426 | 58.398 | 111.878 | 30.205 | 1.00 | 81.86 | 6 |
| ATOM | 6854 | OD1 | ASP | C | 426 | 58.453 | 110.801 | 29.573 | 1.00 | 81.36 | 8 |
| ATOM | 6855 | OD2 | ASP | C | 426 | 57.740 | 112.872 | 29.817 | 1.00 | 80.99 | 8 |
| ATOM | 6856 | C | ASP | C | 426 | 58.237 | 109.757 | 32.193 | 1.00 | 156.95 | 6 |
| ATOM | 6857 | O | ASP | C | 426 | 57.144 | 110.175 | 31.827 | 1.00 | 157.14 | 8 |
| ATOM | 6858 | N | VAL | C | 427 | 58.433 | 108.515 | 32.631 | 1.00 | 169.51 | 7 |
| ATOM | 6859 | CA | VAL | C | 427 | 57.359 | 107.528 | 32.730 | 1.00 | 168.75 | 6 |
| ATOM | 6860 | CB | VAL | C | 427 | 57.885 | 106.173 | 33.236 | 1.00 | 80.98 | 6 |
| ATOM | 6861 | CG1 | VAL | C | 427 | 56.744 | 105.174 | 33.375 | 1.00 | 80.25 | 6 |
| ATOM | 6862 | CG2 | VAL | C | 427 | 58.921 | 105.655 | 32.294 | 1.00 | 81.07 | 6 |
| ATOM | 6863 | C | VAL | C | 427 | 56.307 | 108.007 | 33.714 | 1.00 | 168.67 | 6 |
| ATOM | 6864 | O | VAL | C | 427 | 55.355 | 108.690 | 33.335 | 1.00 | 169.83 | 8 |
| ATOM | 6865 | N | ARG | C | 428 | 56.479 | 107.643 | 34.982 | 1.00 | 89.40 | 7 |
| ATOM | 6866 | CA | ARG | C | 428 | 55.540 | 108.053 | 36.013 | 1.00 | 88.38 | 6 |
| ATOM | 6867 | CB | ARC | C | 428 | 56.003 | 107.524 | 37.372 | 1.00 | 92.50 | 6 |
| ATOM | 6868 | CG | ARG | C | 428 | 56.396 | 106.053 | 37.278 | 1.00 | 93.10 | 6 |
| ATOM | 6869 | CD | ARC | C | 428 | 56.368 | 105.316 | 38.606 | 1.00 | 93.12 | 6 |
| ATOM | 6870 | NE | ARG | C | 428 | 56.960 | 103.984 | 38.474 | 1.00 | 93.43 | 7 |
| ATOM | 6871 | CZ | ARG | C | 428 | 56.875 | 103.024 | 39.390 | 1.00 | 93.85 | 6 |
| ATOM | 6872 | NH1 | ARG | C | 428 | 56.214 | 103.235 | 40.518 | 1.00 | 94.45 | 7 |
| ATOM | 6873 | NH2 | ARC | C | 428 | 57.460 | 101.855 | 39.184 | 1.00 | 94.29 | 7 |
| ATOM | 6874 | C | ARC | C | 428 | 55.473 | 109.575 | 35.974 | 1.00 | 87.64 | 6 |
| ATOM | 6875 | O | ARC | C | 428 | 56.251 | 110.206 | 35.256 | 1.00 | 87.70 | 8 |
| ATOM | 6876 | N | ASP | C | 429 | 54.552 | 110.161 | 36.731 | 1.00 | 110.57 | 7 |
| ATOM | 6877 | CA | ASP | C | 429 | 54.346 | 111.612 | 36.735 | 1.00 | 110.32 | 6 |
| ATOM | 6878 | CB | ASP | C | 429 | 55.643 | 112.413 | 36.476 | 1.00 | 112.49 | 6 |
| ATOM | 6879 | CG | ASP | C | 429 | 56.819 | 111.957 | 37.332 | 1.00 | 113.10 | 6 |
| ATOM | 6880 | OD1 | ASP | C | 429 | 56.609 | 111.595 | 38.508 | 1.00 | 113.98 | 8 |
| ATOM | 6881 | OD2 | ASP | C | 429 | 57.963 | 111.983 | 36.823 | 1.00 | 112.70 | 8 |
| ATOM | 6882 | C | ASP | C | 429 | 53.393 | 111.861 | 35.566 | 1.00 | 109.53 | 6 |
| ATOM | 6883 | O | ASP | C | 429 | 52.361 | 112.516 | 35.712 | 1.00 | 109.81 | 8 |
| ATOM | 6884 | N | VAL | C | 430 | 53.762 | 111.329 | 34.402 | 1.00 | 111.98 | 7 |
| ATOM | 6885 | CA | VAL | C | 430 | 52.964 | 111.459 | 33.187 | 1.00 | 110.45 | 6 |
| ATOM | 6886 | CB | VAL | C | 430 | 53.655 | 110.792 | 31.979 | 1.00 | 68.68 | 6 |
| ATOM | 6887 | CG1 | VAL | C | 430 | 52.653 | 110.543 | 30.862 | 1.00 | 67.72 | 6 |
| ATOM | 6888 | CG2 | VAL | C | 430 | 54.761 | 111.689 | 31.478 | 1.00 | 69.29 | 6 |
| ATOM | 6889 | C | VAL | C | 430 | 51.627 | 110.789 | 33.389 | 1.00 | 109.91 | 6 |
| ATOM | 6890 | O | VAL | C | 430 | 50.587 | 111.449 | 33.363 | 1.00 | 110.52 | 8 |
| ATOM | 6891 | N | HIS | C | 431 | 51.671 | 109.472 | 33.579 | 1.00 | 80.56 | 7 |
| ATOM | 6892 | CA | HIS | C | 431 | 50.471 | 108.672 | 33.797 | 1.00 | 78.57 | 6 |
| ATOM | 6893 | CB | HIS | C | 431 | 50.774 | 107.547 | 34.797 | 1.00 | 58.67 | 6 |
| ATOM | 6894 | CG | HIS | C | 431 | 51.914 | 106.654 | 34.394 | 1.00 | 55.89 | 6 |
| ATOM | 6895 | CD2 | HIS | C | 431 | 52.648 | 105.771 | 35.113 | 1.00 | 55.43 | 6 |
| ATOM | 6896 | ND1 | HIS | C | 431 | 52.351 | 106.538 | 33.092 | 1.00 | 54.61 | 7 |
| ATOM | 6897 | CE1 | HIS | C | 431 | 53.300 | 105.619 | 33.027 | 1.00 | 53.93 | 6 |
| ATOM | 6898 | NE2 | HIS | C | 431 | 53.499 | 105.138 | 34.239 | 1.00 | 53.95 | 7 |
| ATOM | 6899 | C | HIS | C | 431 | 49.320 | 109.545 | 34.328 | 1.00 | 78.25 | 6 |
| ATOM | 6900 | O | HIS | C | 431 | 48.240 | 109.602 | 33.731 | 1.00 | 78.17 | 8 |
| ATOM | 6901 | N | ARG | C | 432 | 49.589 | 110.232 | 35.441 | 1.00 | 46.08 | 7 |
| ATOM | 6902 | CA | ARG | C | 432 | 48.640 | 111.120 | 36.121 | 1.00 | 46.27 | 6 |
| ATOM | 6903 | CB | ARG | C | 432 | 49.373 | 112.334 | 36.700 | 1.00 | 180.77 | 6 |
| ATOM | 6904 | CG | ARG | C | 432 | 50.103 | 112.065 | 38.008 | 1.00 | 183.68 | 6 |
| ATOM | 6905 | CD | ARG | C | 432 | 50.796 | 113.321 | 38.522 | 1.00 | 186.25 | 6 |
| ATOM | 6906 | NE | ARG | C | 432 | 51.412 | 113.112 | 39.830 | 1.00 | 189.10 | 7 |
| ATOM | 6907 | CZ | ARG | C | 432 | 52.165 | 114.013 | 40.453 | 1.00 | 191.25 | 6 |
| ATOM | 6908 | NH1 | ARG | C | 432 | 52.398 | 115.190 | 39.883 | 1.00 | 191.83 | 7 |
| ATOM | 6909 | NH2 | ARG | C | 432 | 52.683 | 113.745 | 41.646 | 1.00 | 192.15 | 7 |
| ATOM | 6910 | C | ARG | C | 432 | 47.473 | 111.614 | 35.294 | 1.00 | 45.55 | 6 |
| ATOM | 6911 | O | ARG | C | 432 | 47.605 | 111.897 | 34.105 | 1.00 | 45.00 | 8 |
| ATOM | 6912 | N | THR | C | 433 | 46.324 | 111.707 | 35.951 | 1.00 | 44.22 | 7 |
| ATOM | 6913 | CA | THR | C | 433 | 45.118 | 112.186 | 35.312 | 1.00 | 44.25 | 6 |
| ATOM | 6914 | CB | THR | C | 433 | 43.924 | 112.264 | 36.301 | 1.00 | 50.03 | 6 |
| ATOM | 6915 | OG1 | THR | C | 433 | 43.121 | 111.092 | 36.165 | 1.00 | 49.50 | 8 |
| ATOM | 6916 | CG2 | TER | C | 433 | 43.054 | 113.474 | 36.021 | 1.00 | 49.59 | 6 |
| ATOM | 6917 | C | THR | C | 433 | 45.404 | 113.574 | 34.776 | 1.00 | 45.40 | 6 |
| ATOM | 6918 | O | THR | C | 433 | 45.919 | 114.447 | 35.486 | 1.00 | 45.43 | 8 |
| ATOM | 6919 | N | HIS | C | 434 | 45.075 | 113.756 | 33.504 | 1.00 | 156.27 | 7 |
| ATOM | 6920 | CA | HIS | C | 434 | 45.254 | 115.025 | 32.826 | 1.00 | 157.07 | 6 |
| ATOM | 6921 | CB | HIS | C | 434 | 45.573 | 114.800 | 31.341 | 1.00 | 144.78 | 6 |
| ATOM | 6922 | CG | HIS | C | 434 | 44.764 | 113.716 | 30.691 | 0.00 | 144.14 | 6 |
| ATOM | 6923 | CD2 | HIS | C | 434 | 43.760 | 112.938 | 31.158 | 0.00 | 143.69 | 6 |
| ATOM | 6924 | ND1 | HIS | C | 434 | 44.982 | 113.319 | 29.389 | 0.00 | 143.69 | 7 |
| ATOM | 6925 | CE1 | HIS | C | 434 | 44.147 | 112.342 | 29.084 | 0.00 | 143.54 | 6 |
| ATOM | 6926 | NE2 | HIS | C | 434 | 43.394 | 112.091 | 30.139 | 0.00 | 143.54 | 7 |
| ATOM | 6927 | C | HIS | C | 434 | 43.980 | 115.830 | 32.970 | 1.00 | 157.44 | 6 |
| ATOM | 6928 | O | HIS | C | 434 | 42.935 | 115.443 | 32.444 | 1.00 | 158.31 | 8 |
| ATOM | 6929 | N | TYR | C | 435 | 44.071 | 116.941 | 33.697 | 1.00 | 67.96 | 7 |
| ATOM | 6930 | CA | TYR | C | 435 | 42.922 | 117.811 | 33.915 | 1.00 | 67.85 | 6 |
| ATOM | 6931 | CB | TYR | C | 435 | 43.379 | 119.169 | 34.456 | 1.00 | 79.02 | 6 |
| ATOM | 6932 | CG | TYR | C | 435 | 44.815 | 119.516 | 34.117 | 0.00 | 78.21 | 6 |
| ATOM | 6933 | CD1 | TYR | C | 435 | 45.238 | 119.612 | 32.792 | 0.00 | 77.88 | 6 |
| ATOM | 6934 | CE1 | TYR | C | 435 | 46.565 | 119.907 | 32.477 | 0.00 | 77.84 | 6 |
| ATOM | 6935 | CD2 | TYR | C | 435 | 45.755 | 119.726 | 35.125 | 0.00 | 77.88 | 6 |
| ATOM | 6936 | CE2 | TYR | C | 435 | 47.084 | 120.022 | 34.823 | 0.00 | 77.84 | 6 |
| ATOM | 6937 | CZ | TYR | C | 435 | 47.482 | 120.109 | 33.498 | 0.00 | 77.79 | 6 |
| ATOM | 6938 | OH | TYR | C | 435 | 48.796 | 120.390 | 33.199 | 0.00 | 78.17 | 8 |
| ATOM | 6939 | C | TYR | C | 435 | 42.102 | 117.978 | 32.636 | 1.00 | 68.74 | 6 |
| ATOM | 6940 | O | TYR | C | 435 | 40.948 | 118.405 | 32.685 | 1.00 | 69.03 | 8 |
| ATOM | 6941 | N | GLY | C | 436 | 42.712 | 117.650 | 31.495 | 1.00 | 67.62 | 7 |
| ATOM | 6942 | CA | GLY | C | 436 | 42.007 | 117.692 | 30.226 | 1.00 | 68.62 | 6 |
| ATOM | 6943 | C | GLY | C | 436 | 41.179 | 116.407 | 30.171 | 1.00 | 69.36 | 6 |
| ATOM | 6944 | O | GLY | C | 436 | 41.598 | 115.412 | 29.585 | 1.00 | 72.11 | 8 |
| ATOM | 6945 | N | ARG | C | 437 | 40.007 | 116.424 | 30.803 | 1.00 | 55.47 | 7 |
| ATOM | 6946 | CA | ARG | C | 437 | 39.119 | 115.263 | 30.855 | 1.00 | 52.65 | 6 |
| ATOM | 6947 | CB | ARG | C | 437 | 38.202 | 115.375 | 32.070 | 1.00 | 51.14 | 6 |
| ATOM | 6948 | CG | ARG | C | 437 | 37.287 | 116.577 | 32.054 | 1.00 | 50.84 | 6 |
| ATOM | 6949 | CD | ARG | C | 437 | 36.395 | 116.602 | 33.275 | 1.00 | 51.08 | 6 |
| ATOM | 6950 | NE | ARG | C | 437 | 35.064 | 117.071 | 32.919 | 1.00 | 52.43 | 7 |
| ATOM | 6951 | CZ | ARG | C | 437 | 34.135 | 117.411 | 33.800 | 1.00 | 53.50 | 6 |
| ATOM | 6952 | NH1 | ARG | C | 437 | 34.389 | 117.337 | 35.096 | 1.00 | 53.97 | 7 |
| ATOM | 6953 | NH2 | ARG | C | 437 | 32.953 | 117.832 | 33.385 | 1.00 | 54.15 | 7 |
| ATOM | 6954 | C | ARG | C | 437 | 38.269 | 115.004 | 29.602 | 1.00 | 52.18 | 6 |
| ATOM | 6955 | O | ARG | C | 437 | 37.055 | 115.243 | 29.574 | 1.00 | 52.84 | 8 |
| ATOM | 6956 | N | ILE | C | 438 | 38.928 | 114.486 | 28.574 | 1.00 | 74.85 | 7 |
| ATOM | 6957 | CA | ILE | C | 438 | 38.304 | 114.161 | 27.298 | 1.00 | 73.00 | 6 |
| ATOM | 6958 | CB | ILE | C | 438 | 38.672 | 115.195 | 26.233 | 1.00 | 47.80 | 6 |
| ATOM | 6959 | CG2 | ILE | C | 438 | 37.485 | 115.435 | 25.387 | 1.00 | 49.03 | 6 |
| ATOM | 6960 | CG1 | ILE | C | 438 | 39.127 | 116.519 | 26.868 | 1.00 | 47.22 | 6 |
| ATOM | 6961 | CD1 | ILE | C | 438 | 39.368 | 117.667 | 25.873 | 1.00 | 44.57 | 6 |
| ATOM | 6962 | C | ILE | C | 438 | 38.913 | 112.820 | 26.900 | 1.00 | 72.42 | 6 |
| ATOM | 6963 | O | ILE | C | 438 | 39.014 | 112.458 | 25.730 | 1.00 | 73.30 | 8 |
| ATOM | 6964 | N | CYS | C | 439 | 39.323 | 112.102 | 27.931 | 1.00 | 77.21 | 7 |
| ATOM | 6965 | CA | CYS | C | 439 | 39.978 | 110.817 | 27.830 | 1.00 | 75.33 | 6 |
| ATOM | 6966 | CB | CYS | C | 439 | 41.477 | 111.086 | 27.736 | 1.00 | 47.34 | 6 |
| ATOM | 6967 | SG | CYS | C | 439 | 42.633 | 109.723 | 27.743 | 1.00 | 37.84 | 16 |
| ATOM | 6968 | C | CYS | C | 439 | 39.591 | 110.191 | 29.165 | 1.00 | 76.68 | 6 |
| ATOM | 6969 | O | CYS | C | 439 | 39.559 | 110.873 | 30.183 | 1.00 | 77.75 | 8 |
| ATOM | 6970 | N | PRO | C | 440 | 39.304 | 108.885 | 29.180 | 1.00 | 34.22 | 7 |
| ATOM | 6971 | CD | PRO | C | 440 | 39.890 | 107.984 | 28.176 | 1.00 | 105.10 | 6 |
| ATOM | 6972 | CA | PRO | C | 440 | 38.896 | 108.117 | 30.369 | 1.00 | 34.23 | 6 |
| ATOM | 6973 | CB | PRO | C | 440 | 39.057 | 106.678 | 29.912 | 1.00 | 105.80 | 6 |
| ATOM | 6974 | CG | PRO | C | 440 | 40.237 | 106.770 | 29.007 | 1.00 | 107.40 | 6 |
| ATOM | 6975 | C | PRO | C | 440 | 39.708 | 108.401 | 31.610 | 1.00 | 34.11 | 6 |
| ATOM | 6976 | O | PRO | C | 440 | 40.929 | 108.486 | 31.545 | 1.00 | 33.92 | 8 |
| ATOM | 6977 | N | VAL | C | 441 | 39.034 | 108.522 | 32.744 | 1.00 | 55.14 | 7 |
| ATOM | 6978 | CA | VAL | C | 441 | 39.731 | 108.816 | 33.981 | 1.00 | 57.66 | 6 |
| ATOM | 6979 | CB | VAL | C | 441 | 38.906 | 109.788 | 34.840 | 1.00 | 86.60 | 6 |
| ATOM | 6980 | CG1 | VAL | C | 441 | 38.932 | 111.162 | 34.223 | 1.00 | 86.91 | 6 |
| ATOM | 6981 | CG2 | VAL | C | 441 | 37.473 | 109.310 | 34.929 | 1.00 | 87.90 | 6 |
| ATOM | 6982 | C | VAL | C | 441 | 40.103 | 107.588 | 34.816 | 1.00 | 59.20 | 6 |
| ATOM | 6983 | O | VAL | C | 441 | 40.921 | 107.690 | 35.736 | 1.00 | 59.36 | 8 |
| ATOM | 6984 | N | GLU | C | 442 | 39.525 | 106.430 | 34.491 | 1.00 | 107.21 | 7 |
| ATOM | 6985 | CA | GLU | C | 442 | 39.809 | 105.199 | 35.235 | 1.00 | 108.59 | 6 |
| ATOM | 6986 | CB | GLU | C | 442 | 38.748 | 104.996 | 36.311 | 1.00 | 86.62 | 6 |
| ATOM | 6987 | CG | GLU | C | 442 | 37.343 | 104.879 | 35.746 | 1.00 | 87.79 | 6 |
| ATOM | 6988 | CD | GLU | C | 442 | 36.302 | 104.513 | 36.795 | 1.00 | 89.95 | 6 |
| ATOM | 6989 | OE1 | GLU | C | 442 | 36.533 | 103.548 | 37.551 | 1.00 | 92.77 | 8 |
| ATOM | 6990 | OE2 | GLU | C | 442 | 35.245 | 105.178 | 36.861 | 1.00 | 89.95 | 8 |
| ATOM | 6991 | C | GLU | C | 442 | 39.860 | 103.948 | 34.353 | 1.00 | 110.16 | 6 |
| ATOM | 6992 | O | GLU | C | 442 | 39.150 | 102.981 | 34.617 | 1.00 | 111.15 | 8 |
| ATOM | 6993 | N | THR | C | 443 | 40.713 | 103.957 | 33.328 | 1.00 | 128.30 | 7 |
| ATOM | 6994 | CA | THR | C | 443 | 40.832 | 102.830 | 32.394 | 1.00 | 129.11 | 6 |
| ATOM | 6995 | CB | THR | C | 443 | 40.634 | 103.300 | 30.929 | 1.00 | 139.96 | 6 |
| ATOM | 6996 | OG1 | THR | C | 443 | 39.325 | 103.854 | 30.775 | 1.00 | 139.87 | 8 |
| ATOM | 6997 | CG2 | THR | C | 443 | 40.777 | 102.138 | 29.969 | 1.00 | 141.01 | 6 |
| ATOM | 6998 | C | THR | C | 443 | 42.125 | 101.999 | 32.422 | 1.00 | 129.49 | 6 |
| ATOM | 6999 | O | THR | C | 443 | 42.080 | 100.783 | 32.247 | 1.00 | 130.53 | 8 |
| ATOM | 7000 | N | PRO | C | 444 | 43.288 | 102.639 | 32.637 | 1.00 | 77.51 | 7 |
| ATOM | 7001 | CD | PRO | C | 444 | 43.423 | 104.024 | 33.120 | 1.00 | 66.72 | 6 |
| ATOM | 7002 | CA | PRO | C | 444 | 44.590 | 101.959 | 32.675 | 1.00 | 78.03 | 6 |
| ATOM | 7003 | CB | PRO | C | 444 | 45.446 | 102.908 | 33.507 | 1.00 | 67.09 | 6 |
| ATOM | 7004 | CG | PRO | C | 444 | 44.922 | 104.228 | 33.138 | 1.00 | 66.86 | 6 |
| ATOM | 7005 | C | PRO | C | 444 | 44.581 | 100.560 | 33.270 | 1.00 | 78.64 | 6 |
| ATOM | 7006 | O | PRO | C | 444 | 43.617 | 100.162 | 33.918 | 1.00 | 80.74 | 8 |
| ATOM | 7007 | N | GLU | C | 445 | 45.657 | 99.812 | 33.053 | 1.00 | 35.81 | 7 |
| ATOM | 7008 | CA | GLU | C | 445 | 45.733 | 98.482 | 33.634 | 1.00 | 35.36 | 6 |
| ATOM | 7009 | CB | GLU | C | 445 | 45.944 | 97.440 | 32.537 | 1.00 | 77.62 | 6 |
| ATOM | 7010 | CG | GLU | C | 445 | 44.692 | 96.612 | 32.246 | 1.00 | 79.75 | 6 |
| ATOM | 7011 | CD | GLU | C | 445 | 44.799 | 95.175 | 32.736 | 1.00 | 80.05 | 6 |
| ATOM | 7012 | OE1 | GLU | C | 445 | 45.667 | 94.434 | 32.235 | 1.00 | 80.64 | 8 |
| ATOM | 7013 | OE2 | GLU | C | 445 | 44.015 | 94.784 | 33.619 | 1.00 | 81.30 | 8 |
| ATOM | 7014 | C | GLU | C | 445 | 46.796 | 98.339 | 34.751 | 1.00 | 34.05 | 6 |
| ATOM | 7015 | O | GLU | C | 445 | 46.697 | 98.974 | 35.797 | 1.00 | 34.45 | 8 |
| ATOM | 7016 | N | GLY | C | 446 | 47.799 | 97.493 | 34.554 | 1.00 | 107.56 | 7 |
| ATOM | 7017 | CA | GLY | C | 446 | 48.804 | 97.328 | 35.593 | 1.00 | 107.12 | 6 |
| ATOM | 7018 | C | GLY | C | 446 | 50.242 | 97.318 | 35.103 | 1.00 | 106.84 | 6 |
| ATOM | 7019 | O | GLY | C | 446 | 50.555 | 96.730 | 34.062 | 1.00 | 108.10 | 8 |
| ATOM | 7020 | N | ALA | C | 447 | 51.122 | 97.972 | 35.857 | 1.00 | 30.72 | 7 |
| ATOM | 7021 | CA | ALA | C | 447 | 52.540 | 98.037 | 35.513 | 1.00 | 28.70 | 6 |
| ATOM | 7022 | CB | ALA | C | 447 | 53.145 | 96.629 | 35.534 | 1.00 | 121.82 | 6 |
| ATOM | 7023 | C | ALA | C | 447 | 52.753 | 98.689 | 34.145 | 1.00 | 27.10 | 6 |
| ATOM | 7024 | O | ALA | C | 447 | 53.863 | 98.708 | 33.607 | 1.00 | 26.16 | 8 |
| ATOM | 7025 | N | ASN | C | 448 | 51.670 | 99.231 | 33.605 | 1.00 | 30.81 | 7 |
| ATOM | 7026 | CA | ASN | C | 448 | 51.650 | 99.891 | 32.305 | 1.00 | 29.38 | 6 |
| ATOM | 7027 | CB | ASN | C | 448 | 51.067 | 98.926 | 31.261 | 1.00 | 61.60 | 6 |
| ATOM | 7028 | CG | ASN | C | 448 | 51.307 | 99.381 | 29.845 | 1.00 | 64.54 | 6 |
| ATOM | 7029 | OD1 | ASN | C | 448 | 51.317 | 100.567 | 29.561 | 1.00 | 68.29 | 8 |
| ATOM | 7030 | ND2 | ASN | C | 448 | 51.487 | 98.435 | 28.944 | 1.00 | 65.27 | 7 |
| ATOM | 7031 | C | ASN | C | 448 | 50.719 | 101.102 | 32.466 | 1.00 | 27.57 | 6 |
| ATOM | 7032 | O | ASN | C | 448 | 50.221 | 101.644 | 31.487 | 1.00 | 26.46 | 8 |
| ATOM | 7033 | N | ILE | C | 449 | 50.486 | 101.505 | 33.716 | 1.00 | 71.41 | 7 |
| ATOM | 7034 | CA | ILE | C | 449 | 49.587 | 102.615 | 34.059 | 1.00 | 71.54 | 6 |
| ATOM | 7035 | CB | ILE | C | 449 | 49.902 | 103.160 | 35.467 | 1.00 | 138.02 | 6 |
| ATOM | 7036 | CG2 | ILE | C | 449 | 48.723 | 103.956 | 35.986 | 1.00 | 138.57 | 6 |
| ATOM | 7037 | CG1 | ILE | C | 449 | 50.209 | 102.004 | 36.422 | 1.00 | 138.34 | 6 |
| ATOM | 7038 | CD1 | ILE | C | 449 | 49.134 | 100.939 | 36.475 | 1.00 | 139.38 | 6 |
| ATOM | 7039 | C | ILE | C | 449 | 49.627 | 103.792 | 33.087 | 1.00 | 70.75 | 6 |
| ATOM | 7040 | O | ILE | C | 449 | 50.579 | 103.946 | 32.329 | 1.00 | 71.98 | 8 |
| ATOM | 7041 | N | GLY | C | 450 | 48.579 | 104.614 | 33.116 | 1.00 | 82.97 | 7 |
| ATOM | 7042 | CA | GLY | C | 450 | 48.502 | 105.798 | 32.268 | 1.00 | 80.93 | 6 |
| ATOM | 7043 | C | GLY | C | 450 | 48.743 | 105.648 | 30.778 | 1.00 | 79.00 | 6 |
| ATOM | 7044 | O | GLY | C | 450 | 47.830 | 105.815 | 29.976 | 1.00 | 79.47 | 8 |
| ATOM | 7045 | N | LEU | C | 451 | 49.982 | 105.359 | 30.405 | 1.00 | 53.93 | 7 |
| ATOM | 7046 | CA | LEU | C | 451 | 50.367 | 105.186 | 29.007 | 1.00 | 52.47 | 6 |
| ATOM | 7047 | CB | LEU | C | 451 | 51.494 | 104.173 | 28.908 | 1.00 | 30.07 | 6 |
| ATOM | 7048 | CG | LEU | C | 451 | 52.689 | 104.410 | 29.809 | 1.00 | 29.20 | 6 |
| ATOM | 7049 | CD1 | LEU | C | 451 | 53.644 | 103.265 | 29.605 | 1.00 | 28.64 | 6 |
| ATOM | 7050 | CD2 | LEU | C | 451 | 53.342 | 105.743 | 29.498 | 1.00 | 28.84 | 6 |
| ATOM | 7051 | C | LEU | C | 451 | 49.237 | 104.724 | 28.093 | 1.00 | 52.53 | 6 |
| ATOM | 7052 | O | LEU | C | 451 | 49.004 | 105.303 | 27.033 | 1.00 | 52.57 | 8 |
| ATOM | 7053 | N | ILE | C | 452 | 48.547 | 103.667 | 28.505 | 1.00 | 37.93 | 7 |
| ATOM | 7054 | CA | ILE | C | 452 | 47.466 | 103.115 | 27.713 | 1.00 | 39.02 | 6 |
| ATOM | 7055 | CB | ILE | C | 452 | 47.519 | 101.621 | 27.717 | 1.00 | 49.63 | 6 |
| ATOM | 7056 | CG2 | ILE | C | 452 | 46.469 | 101.088 | 26.781 | 1.00 | 51.01 | 6 |
| ATOM | 7057 | CG1 | ILE | C | 452 | 48.910 | 101.167 | 27.300 | 1.00 | 51.02 | 6 |
| ATOM | 7058 | CD1 | ILE | C | 452 | 49.150 | 99.705 | 27.517 | 1.00 | 52.72 | 6 |
| ATOM | 7059 | C | ILE | C | 452 | 46.094 | 103.515 | 28.200 | 1.00 | 39.38 | 6 |
| ATOM | 7060 | O | ILE | C | 452 | 45.488 | 102.826 | 29.012 | 1.00 | 40.50 | 8 |
| ATOM | 7061 | N | THR | C | 453 | 45.591 | 104.621 | 27.680 | 1.00 | 42.99 | 7 |
| ATOM | 7062 | CA | THR | C | 453 | 44.286 | 105.104 | 28.083 | 1.00 | 43.18 | 6 |
| ATOM | 7063 | CB | THR | C | 453 | 44.306 | 106.628 | 28.190 | 1.00 | 78.48 | 6 |
| ATOM | 7064 | OG1 | THR | C | 453 | 42.988 | 107.100 | 28.474 | 1.00 | 82.01 | 8 |
| ATOM | 7065 | CG2 | THR | C | 453 | 44.820 | 107.248 | 26.898 | 1.00 | 77.30 | 6 |
| ATOM | 7066 | C | THR | C | 453 | 43.214 | 104.675 | 27.093 | 1.00 | 43.15 | 6 |
| ATOM | 7067 | O | THR | C | 453 | 43.518 | 104.039 | 26.077 | 1.00 | 43.49 | 8 |
| ATOM | 7068 | N | SER | C | 454 | 41.963 | 105.017 | 27.397 | 1.00 | 45.38 | 7 |
| ATOM | 7069 | CA | SER | C | 454 | 40.850 | 104.674 | 26.524 | 1.00 | 45.94 | 6 |
| ATOM | 7070 | CB | SER | C | 454 | 39.728 | 104.016 | 27.325 | 1.00 | 81.44 | 6 |
| ATOM | 7071 | OG | SER | C | 454 | 38.829 | 103.337 | 26.460 | 1.00 | 86.20 | 8 |
| ATOM | 7072 | C | SER | C | 454 | 40.330 | 105.916 | 25.801 | 1.00 | 45.54 | 6 |
| ATOM | 7073 | O | SER | C | 454 | 41.045 | 106.497 | 24.995 | 1.00 | 46.42 | 8 |
| ATOM | 7074 | N | LEU | C | 455 | 39.099 | 106.339 | 26.076 | 1.00 | 48.01 | 7 |
| ATOM | 7075 | CA | LEU | C | 455 | 38.560 | 107.513 | 25.396 | 1.00 | 47.92 | 6 |
| ATOM | 7076 | CB | LEU | C | 455 | 38.567 | 107.277 | 23.889 | 1.00 | 34.19 | 6 |
| ATOM | 7077 | CG | LEU | C | 455 | 38.176 | 108.510 | 23.095 | 1.00 | 35.88 | 6 |
| ATOM | 7078 | CD1 | LEU | C | 455 | 39.200 | 109.570 | 23.404 | 1.00 | 36.69 | 6 |
| ATOM | 7079 | CD2 | LEU | C | 455 | 38.119 | 108.232 | 21.613 | 1.00 | 35.89 | 6 |
| ATOM | 7080 | C | LEU | C | 455 | 37.146 | 107.889 | 25.835 | 1.00 | 47.75 | 6 |
| ATOM | 7081 | O | LEU | C | 455 | 36.266 | 107.033 | 25.900 | 1.00 | 47.58 | 8 |
| ATOM | 7082 | N | ALA | C | 456 | 36.938 | 109.177 | 26.118 | 1.00 | 47.48 | 7 |
| ATOM | 7083 | CA | ALA | C | 456 | 35.633 | 109.702 | 26.551 | 1.00 | 46.84 | 6 |
| ATOM | 7084 | CB | ALA | C | 456 | 35.653 | 111.226 | 26.601 | 1.00 | 16.72 | 6 |
| ATOM | 7085 | C | ALA | C | 456 | 34.543 | 109.262 | 25.616 | 1.00 | 46.95 | 6 |
| ATOM | 7086 | O | ALA | C | 456 | 34.716 | 109.289 | 24.402 | 1.00 | 47.71 | 8 |
| ATOM | 7087 | N | ALA | C | 457 | 33.408 | 108.883 | 26.177 | 1.00 | 39.38 | 7 |
| ATOM | 7088 | CA | ALA | C | 457 | 32.315 | 108.433 | 25.350 | 1.00 | 39.45 | 6 |
| ATOM | 7089 | CB | ALA | C | 457 | 31.103 | 108.211 | 26.187 | 1.00 | 13.87 | 6 |
| ATOM | 7090 | C | ALA | C | 457 | 32.023 | 109.441 | 24.263 | 1.00 | 39.11 | 6 |
| ATOM | 7091 | O | ALA | C | 457 | 32.367 | 109.250 | 23.105 | 1.00 | 39.87 | 8 |
| ATOM | 7092 | N | TYR | C | 458 | 31.403 | 110.535 | 24.648 | 1.00 | 36.08 | 7 |
| ATOM | 7093 | CA | TYR | C | 458 | 31.044 | 111.565 | 23.701 | 1.00 | 37.92 | 6 |
| ATOM | 7094 | CB | TYR | C | 458 | 30.203 | 112.617 | 24.412 | 1.00 | 33.09 | 6 |
| ATOM | 7095 | CG | TYR | C | 458 | 28.740 | 112.305 | 24.598 | 1.00 | 31.68 | 6 |
| ATOM | 7096 | CD1 | TYR | C | 458 | 28.315 | 111.161 | 25.264 | 1.00 | 31.59 | 6 |
| ATOM | 7097 | CE1 | TYR | C | 458 | 26.974 | 110.917 | 25.476 | 1.00 | 30.71 | 6 |
| ATOM | 7098 | CD2 | TYR | C | 458 | 27.782 | 113.191 | 24.150 | 1.00 | 31.70 | 6 |
| ATOM | 7099 | CE2 | TYR | C | 458 | 26.458 | 112.963 | 24.354 | 1.00 | 31.63 | 6 |
| ATOM | 7100 | CZ | TYR | C | 458 | 26.052 | 111.832 | 25.013 | 1.00 | 31.31 | 6 |
| ATOM | 7101 | OH | TYR | C | 458 | 24.705 | 111.627 | 25.183 | 1.00 | 32.83 | 8 |
| ATOM | 7102 | C | TYR | C | 458 | 32.224 | 112.273 | 23.031 | 1.00 | 40.86 | 6 |
| ATOM | 7103 | O | TYR | C | 458 | 31.997 | 113.158 | 22.206 | 1.00 | 41.46 | 8 |
| ATOM | 7104 | N | ALA | C | 459 | 33.460 | 111.894 | 23.372 | 1.00 | 61.61 | 7 |
| ATOM | 7105 | CA | ALA | C | 459 | 34.681 | 112.541 | 22.852 | 1.00 | 63.60 | 6 |
| ATOM | 7106 | CB | ALA | C | 459 | 35.901 | 111.926 | 23.525 | 1.00 | 181.11 | 6 |
| ATOM | 7107 | C | ALA | C | 459 | 34.948 | 112.643 | 21.349 | 1.00 | 64.59 | 6 |
| ATOM | 7108 | O | ALA | C | 459 | 34.663 | 111.733 | 20.572 | 1.00 | 65.49 | 8 |
| ATOM | 7109 | N | ARG | C | 460 | 35.532 | 113.772 | 20.967 | 1.00 | 66.70 | 7 |
| ATOM | 7110 | CA | ARG | C | 460 | 35.904 | 114.078 | 19.588 | 1.00 | 68.41 | 6 |
| ATOM | 7111 | CB | ARG | C | 460 | 37.184 | 113.342 | 19.205 | 1.00 | 52.09 | 6 |
| ATOM | 7112 | CG | ARG | C | 460 | 37.435 | 113.406 | 17.721 | 1.00 | 50.99 | 6 |
| ATOM | 7113 | CD | ARG | C | 460 | 38.894 | 113.455 | 17.377 | 1.00 | 50.98 | 6 |
| ATOM | 7114 | NE | ARG | C | 460 | 39.647 | 112.360 | 17.975 | 1.00 | 52.48 | 7 |
| ATOM | 7115 | CZ | ARG | C | 460 | 40.920 | 112.086 | 17.692 | 1.00 | 52.62 | 6 |
| ATOM | 7116 | NH1 | ARG | C | 460 | 41.574 | 112.830 | 16.811 | 1.00 | 51.72 | 7 |
| ATOM | 7117 | NH2 | ARG | C | 460 | 41.547 | 111.082 | 18.302 | 1.00 | 53.37 | 7 |
| ATOM | 7118 | C | ARG | C | 460 | 34.921 | 113.891 | 18.446 | 1.00 | 70.46 | 6 |
| ATOM | 7119 | O | ARG | C | 460 | 34.249 | 112.865 | 18.349 | 1.00 | 70.85 | 8 |
| ATOM | 7120 | N | VAL | C | 461 | 34.905 | 114.898 | 17.565 | 1.00 | 174.70 | 7 |
| ATOM | 7121 | CA | VAL | C | 461 | 34.087 | 114.968 | 16.346 | 1.00 | 175.02 | 6 |
| ATOM | 7122 | CB | VAL | C | 461 | 32.671 | 114.371 | 16.535 | 1.00 | 46.89 | 6 |
| ATOM | 7123 | CG1 | VAL | C | 461 | 31.623 | 115.263 | 15.908 | 1.00 | 47.41 | 6 |
| ATOM | 7124 | CG2 | VAL | C | 461 | 32.607 | 113.039 | 15.853 | 1.00 | 45.72 | 6 |
| ATOM | 7125 | C | VAL | C | 461 | 33.947 | 116.401 | 15.824 | 1.00 | 175.88 | 6 |
| ATOM | 7126 | O | VAL | C | 461 | 33.629 | 117.314 | 16.586 | 1.00 | 176.25 | 8 |
| ATOM | 7127 | N | ALA | C | 462 | 34.181 | 116.564 | 14.519 | 1.00 | 122.51 | 7 |
| ATOM | 7128 | CA | ALA | C | 462 | 34.112 | 117.845 | 13.796 | 1.00 | 123.73 | 6 |
| ATOM | 7129 | CB | ALA | C | 462 | 33.877 | 119.005 | 14.752 | 1.00 | 28.29 | 6 |
| ATOM | 7130 | C | ALA | C | 462 | 35.426 | 118.052 | 13.046 | 1.00 | 125.07 | 6 |
| ATOM | 7131 | O | ALA | C | 462 | 36.335 | 117.228 | 13.165 | 1.00 | 125.53 | 8 |
| ATOM | 7132 | N | ALA | C | 463 | 35.527 | 119.137 | 12.273 | 1.00 | 128.67 | 7 |
| ATOM | 7133 | CA | ALA | C | 463 | 36.752 | 119.423 | 11.522 | 1.00 | 128.13 | 6 |
| ATOM | 7134 | CB | ALA | C | 463 | 36.757 | 120.887 | 11.040 | 1.00 | 50.08 | 6 |
| ATOM | 7135 | C | ALA | C | 463 | 37.923 | 119.147 | 12.469 | 1.00 | 127.82 | 6 |
| ATOM | 7136 | O | ALA | C | 463 | 38.264 | 119.978 | 13.318 | 1.00 | 128.71 | 8 |
| ATOM | 7137 | N | LEU | C | 464 | 38.525 | 117.967 | 12.313 | 1.00 | 54.63 | 7 |
| ATOM | 7138 | CA | LEU | C | 464 | 39.616 | 117.507 | 13.185 | 1.00 | 54.40 | 6 |
| ATOM | 7139 | CB | LEU | C | 464 | 39.453 | 116.008 | 13.435 | 1.00 | 112.52 | 6 |
| ATOM | 7140 | CG | LEU | C | 464 | 40.430 | 115.419 | 14.447 | 1.00 | 113.36 | 6 |
| ATOM | 7141 | CD1 | LEU | C | 464 | 40.184 | 116.035 | 15.817 | 1.00 | 114.51 | 6 |
| ATOM | 7142 | CD2 | LEU | C | 464 | 40.258 | 113.913 | 14.487 | 1.00 | 114.04 | 6 |
| ATOM | 7143 | C | LEU | C | 464 | 41.089 | 117.786 | 12.838 | 1.00 | 53.28 | 6 |
| ATOM | 7144 | O | LEU | C | 464 | 41.760 | 117.000 | 12.154 | 1.00 | 51.53 | 8 |
| ATOM | 7145 | N | GLY | C | 465 | 41.581 | 118.912 | 13.348 | 1.00 | 98.23 | 7 |
| ATOM | 7146 | CA | GLY | C | 465 | 42.966 | 119.278 | 13.148 | 1.00 | 96.83 | 6 |
| ATOM | 7147 | C | GLY | C | 465 | 43.717 | 118.426 | 14.147 | 1.00 | 96.13 | 6 |
| ATOM | 7148 | O | GLY | C | 465 | 44.832 | 117.980 | 13.879 | 1.00 | 95.71 | 8 |
| ATOM | 7149 | N | PHE | C | 466 | 43.074 | 118.196 | 15.298 | 1.00 | 84.96 | 7 |
| ATOM | 7150 | CA | PHE | C | 466 | 43.628 | 117.378 | 16.383 | 1.00 | 82.91 | 6 |
| ATOM | 7151 | CB | PHE | C | 466 | 45.070 | 117.811 | 16.710 | 1.00 | 75.66 | 6 |
| ATOM | 7152 | CG | PHE | C | 466 | 45.199 | 119.201 | 17.341 | 1.00 | 75.74 | 6 |
| ATOM | 7153 | CD1 | PHE | C | 466 | 44.847 | 119.421 | 18.669 | 1.00 | 75.04 | 6 |
| ATOM | 7154 | CD2 | PHE | C | 466 | 45.756 | 120.265 | 16.619 | 1.00 | 75.64 | 6 |
| ATOM | 7155 | CE1 | PHE | C | 466 | 45.052 | 120.656 | 19.272 | 1.00 | 75.12 | 6 |
| ATOM | 7156 | CE2 | PHE | C | 466 | 45.962 | 121.511 | 17.220 | 1.00 | 74.81 | 6 |
| ATOM | 7157 | CZ | PHE | C | 466 | 45.612 | 121.702 | 18.547 | 1.00 | 75.38 | 6 |
| ATOM | 7158 | C | PHE | C | 466 | 42.810 | 117.348 | 17.677 | 1.00 | 81.00 | 6 |
| ATOM | 7159 | O | PHE | C | 466 | 41.709 | 117.890 | 17.756 | 1.00 | 80.79 | 8 |
| ATOM | 7160 | N | ILE | C | 467 | 43.395 | 116.681 | 18.667 | 1.00 | 42.94 | 7 |
| ATOM | 7161 | CA | ILE | C | 467 | 42.888 | 116.482 | 20.033 | 1.00 | 41.29 | 6 |
| ATOM | 7162 | CB | ILE | C | 467 | 43.575 | 117.476 | 20.973 | 1.00 | 110.42 | 6 |
| ATOM | 7163 | CG2 | ILE | C | 467 | 43.261 | 117.123 | 22.413 | 1.00 | 109.50 | 6 |
| ATOM | 7164 | CG1 | ILE | C | 467 | 45.090 | 117.367 | 20.775 | 1.00 | 112.81 | 6 |
| ATOM | 7165 | CD1 | ILE | C | 467 | 45.907 | 118.427 | 21.464 | 1.00 | 116.18 | 6 |
| ATOM | 7166 | C | ILE | C | 467 | 41.403 | 116.366 | 20.449 | 1.00 | 38.46 | 6 |
| ATOM | 7167 | O | ILE | C | 467 | 40.613 | 117.291 | 20.366 | 1.00 | 37.83 | 8 |
| ATOM | 7168 | N | ARG | C | 468 | 41.089 | 115.184 | 20.960 | 1.00 | 64.17 | 7 |
| ATOM | 7169 | CA | ARG | C | 468 | 39.781 | 114.749 | 21.437 | 1.00 | 61.14 | 6 |
| ATOM | 7170 | CB | ARG | C | 468 | 39.993 | 113.970 | 22.723 | 1.00 | 65.30 | 6 |
| ATOM | 7171 | CG | ARG | C | 468 | 41.068 | 112.935 | 22.573 | 1.00 | 63.34 | 6 |
| ATOM | 7172 | CD | ARG | C | 468 | 41.819 | 112.761 | 23.857 | 1.00 | 62.63 | 6 |
| ATOM | 7173 | NE | ARG | C | 468 | 42.908 | 111.817 | 23.679 | 1.00 | 62.42 | 7 |
| ATOM | 7174 | CZ | ARG | C | 468 | 43.681 | 111.375 | 24.662 | 1.00 | 62.87 | 6 |
| ATOM | 7175 | NH1 | ARG | C | 468 | 43.487 | 111.796 | 25.906 | 1.00 | 62.17 | 7 |
| ATOM | 7176 | NH2 | ARG | C | 468 | 44.647 | 110.505 | 24.398 | 1.00 | 62.03 | 7 |
| ATOM | 7177 | C | ARG | C | 468 | 38.578 | 115.666 | 21.618 | 1.00 | 59.37 | 6 |
| ATOM | 7178 | O | ARG | C | 468 | 37.467 | 115.218 | 21.389 | 1.00 | 59.52 | 8 |
| ATOM | 7179 | N | THR | C | 469 | 38.762 | 116.911 | 22.046 | 1.00 | 28.37 | 7 |
| ATOM | 7180 | CA | THR | C | 469 | 37.618 | 117.830 | 22.247 | 1.00 | 27.33 | 6 |
| ATOM | 7181 | CB | THR | C | 469 | 37.348 | 118.700 | 20.995 | 1.00 | 77.07 | 6 |
| ATOM | 7182 | OG1 | THR | C | 469 | 36.958 | 117.865 | 19.905 | 1.00 | 77.37 | 8 |
| ATOM | 7183 | CG2 | THR | C | 469 | 38.578 | 119.489 | 20.600 | 1.00 | 77.50 | 6 |
| ATOM | 7184 | C | THR | C | 469 | 36.261 | 117.182 | 22.627 | 1.00 | 25.69 | 6 |
| ATOM | 7185 | O | THR | C | 469 | 35.666 | 116.455 | 21.828 | 1.00 | 25.93 | 8 |
| ATOM | 7186 | N | PRO | C | 470 | 35.737 | 117.473 | 23.842 | 1.00 | 39.55 | 7 |
| ATOM | 7187 | CD | PRO | C | 470 | 36.260 | 118.385 | 24.884 | 1.00 | 24.20 | 6 |
| ATOM | 7188 | CA | PRO | C | 470 | 34.457 | 116.892 | 24.268 | 1.00 | 38.89 | 6 |
| ATOM | 7189 | CB | PRO | C | 470 | 34.534 | 116.963 | 25.779 | 1.00 | 22.75 | 6 |
| ATOM | 7190 | CG | PRO | C | 470 | 35.172 | 118.308 | 25.980 | 1.00 | 22.38 | 6 |
| ATOM | 7191 | C | PRO | C | 470 | 33.304 | 117.716 | 23.741 | 1.00 | 39.28 | 6 |
| ATOM | 7192 | O | PRO | C | 470 | 33.425 | 118.405 | 22.726 | 1.00 | 41.11 | 8 |
| ATOM | 7193 | N | TYR | C | 471 | 32.182 | 117.654 | 24.437 | 1.00 | 32.55 | 7 |
| ATOM | 7194 | CA | TYR | C | 471 | 31.016 | 118.403 | 24.025 | 1.00 | 34.52 | 6 |
| ATOM | 7195 | CB | TYR | C | 471 | 29.879 | 117.433 | 23.683 | 1.00 | 75.03 | 6 |
| ATOM | 7196 | CG | TYR | C | 471 | 30.055 | 116.756 | 22.333 | 1.00 | 77.73 | 6 |
| ATOM | 7197 | CD1 | TYR | C | 471 | 31.199 | 116.012 | 22.041 | 1.00 | 77.86 | 6 |
| ATOM | 7198 | CE1 | TYR | C | 471 | 31.398 | 115.467 | 20.777 | 1.00 | 78.14 | 6 |
| ATOM | 7199 | CD2 | TYR | C | 471 | 29.109 | 116.923 | 21.319 | 1.00 | 78.93 | 6 |
| ATOM | 7200 | CE2 | TYR | C | 471 | 29.305 | 116.376 | 20.052 | 1.00 | 78.86 | 6 |
| ATOM | 7201 | CZ | TYR | C | 471 | 30.449 | 115.656 | 19.794 | 1.00 | 78.86 | 6 |
| ATOM | 7202 | OH | TYR | C | 471 | 30.646 | 115.138 | 18.543 | 1.00 | 80.15 | 8 |
| ATOM | 7203 | C | TYR | C | 471 | 30.626 | 119.316 | 25.162 | 1.00 | 35.18 | 6 |
| ATOM | 7204 | O | TYR | C | 471 | 30.895 | 119.013 | 26.321 | 1.00 | 35.81 | 8 |
| ATOM | 7205 | N | ARG | C | 472 | 30.017 | 120.450 | 24.841 | 1.00 | 59.06 | 7 |
| ATOM | 7206 | CA | ARG | C | 472 | 29.589 | 121.380 | 25.876 | 1.00 | 59.21 | 6 |
| ATOM | 7207 | CB | ARG | C | 472 | 30.739 | 122.306 | 26.280 | 1.00 | 47.34 | 6 |
| ATOM | 7208 | CG | ARG | C | 472 | 32.014 | 121.578 | 26.692 | 1.00 | 46.47 | 6 |
| ATOM | 7209 | CD | ARG | C | 472 | 32.857 | 122.380 | 27.671 | 1.00 | 46.11 | 6 |
| ATOM | 7210 | NE | ARG | C | 472 | 34.206 | 121.839 | 27.775 | 1.00 | 47.37 | 7 |
| ATOM | 7211 | CZ | ARG | C | 472 | 34.486 | 120.594 | 28.139 | 1.00 | 47.44 | 6 |
| ATOM | 7212 | NH1 | ARG | C | 472 | 33.499 | 119.766 | 28.436 | 1.00 | 46.95 | 7 |
| ATOM | 7213 | NH2 | ARG | C | 472 | 35.748 | 120.175 | 28.196 | 1.00 | 47.86 | 7 |
| ATOM | 7214 | C | ARG | C | 472 | 28.403 | 122.210 | 25.414 | 1.00 | 60.34 | 6 |
| ATOM | 7215 | O | ARG | C | 472 | 28.386 | 122.726 | 24.301 | 1.00 | 61.28 | 8 |
| ATOM | 7216 | N | ARG | C | 473 | 27.403 | 122.330 | 26.272 | 1.00 | 55.07 | 7 |
| ATOM | 7217 | CA | ARC | C | 473 | 26.227 | 123.115 | 25.943 | 1.00 | 56.77 | 6 |
| ATOM | 7218 | CB | ARG | C | 473 | 25.078 | 122.723 | 26.869 | 1.00 | 63.20 | 6 |
| ATOM | 7219 | CG | ARG | C | 473 | 23.718 | 122.857 | 26.235 | 1.00 | 63.69 | 6 |
| ATOM | 7220 | CD | ARG | C | 473 | 22.651 | 122.179 | 27.073 | 1.00 | 64.69 | 6 |
| ATOM | 7221 | NE | ARG | C | 473 | 21.331 | 122.309 | 26.459 | 1.00 | 66.59 | 7 |
| ATOM | 7222 | CZ | ARG | C | 473 | 20.197 | 121.877 | 27.007 | 1.00 | 67.61 | 6 |
| ATOM | 7223 | NH1 | ARG | C | 473 | 20.222 | 121.278 | 28.194 | 1.00 | 68.38 | 7 |
| ATOM | 7224 | NH2 | ARG | C | 473 | 19.035 | 122.055 | 26.378 | 1.00 | 66.88 | 7 |
| ATOM | 7225 | C | ARG | C | 473 | 26.575 | 124.604 | 26.091 | 1.00 | 58.13 | 6 |
| ATOM | 7226 | O | ARG | C | 473 | 27.644 | 124.956 | 26.584 | 1.00 | 58.42 | 8 |
| ATOM | 7227 | N | VAL | C | 474 | 25.678 | 125.474 | 25.651 | 1.00 | 43.60 | 7 |
| ATOM | 7228 | CA | VAL | C | 474 | 25.880 | 126.924 | 25.700 | 1.00 | 45.65 | 6 |
| ATOM | 7229 | CB | VAL | C | 474 | 26.764 | 127.410 | 24.510 | 1.00 | 105.12 | 6 |
| ATOM | 7230 | CG1 | VAL | C | 474 | 26.961 | 128.919 | 24.573 | 1.00 | 107.36 | 6 |
| ATOM | 7231 | CG2 | VAL | C | 474 | 28.103 | 126.694 | 24.511 | 1.00 | 104.39 | 6 |
| ATOM | 7232 | C | VAL | C | 474 | 24.482 | 127.505 | 25.517 | 1.00 | 46.42 | 6 |
| ATOM | 7233 | O | VAL | C | 474 | 24.228 | 128.288 | 24.591 | 1.00 | 46.13 | 8 |
| ATOM | 7234 | N | ALA | C | 475 | 23.572 | 127.085 | 26.390 | 1.00 | 68.26 | 7 |
| ATOM | 7235 | CA | ALA | C | 475 | 22.171 | 127.507 | 26.346 | 1.00 | 69.34 | 6 |
| ATOM | 7236 | CB | ALA | C | 475 | 22.074 | 129.017 | 26.224 | 1.00 | 168.36 | 6 |
| ATOM | 7237 | C | ALA | C | 475 | 21.429 | 126.837 | 25.188 | 1.00 | 69.26 | 6 |
| ATOM | 7238 | O | ALA | C | 475 | 21.237 | 127.431 | 24.121 | 1.00 | 67.34 | 8 |
| ATOM | 7239 | N | ALA | C | 476 | 21.031 | 125.590 | 25.422 | 1.00 | 62.24 | 7 |
| ATOM | 7240 | CA | ALA | C | 476 | 20.295 | 124.784 | 24.455 | 1.00 | 64.22 | 6 |
| ATOM | 7241 | CB | ALA | C | 476 | 18.887 | 125.337 | 24.294 | 1.00 | 146.64 | 6 |
| ATOM | 7242 | C | ALA | C | 476 | 20.969 | 124.690 | 23.098 | 1.00 | 65.26 | 6 |
| ATOM | 7243 | O | ALA | C | 476 | 20.937 | 123.640 | 22.453 | 1.00 | 64.43 | 8 |
| ATOM | 7244 | N | GLY | C | 477 | 21.561 | 125.802 | 22.677 | 1.00 | 116.21 | 7 |
| ATOM | 7245 | CA | GLY | C | 477 | 22.239 | 125.890 | 21.399 | 1.00 | 118.42 | 6 |
| ATOM | 7246 | C | GLY | C | 477 | 22.437 | 124.574 | 20.684 | 1.00 | 118.96 | 6 |
| ATOM | 7247 | O | GLY | C | 477 | 21.511 | 124.034 | 20.075 | 1.00 | 119.12 | 8 |
| ATOM | 7248 | N | ALA | C | 478 | 23.654 | 124.057 | 20.761 | 1.00 | 84.50 | 7 |
| ATOM | 7249 | CA | ALA | C | 478 | 23.970 | 122.803 | 20.109 | 1.00 | 85.41 | 6 |
| ATOM | 7250 | CB | ALA | C | 478 | 24.664 | 123.065 | 18.782 | 1.00 | 170.39 | 6 |
| ATOM | 7251 | C | ALA | C | 478 | 24.859 | 121.962 | 21.001 | 1.00 | 85.57 | 6 |
| ATOM | 7252 | O | ALA | C | 478 | 24.391 | 121.074 | 21.704 | 1.00 | 84.87 | 8 |
| ATOM | 7253 | N | VAL | C | 479 | 26.149 | 122.247 | 20.966 | 1.00 | 65.43 | 7 |
| ATOM | 7254 | CA | VAL | C | 479 | 27.096 | 121.505 | 21.766 | 1.00 | 66.60 | 6 |
| ATOM | 7255 | CB | VAL | C | 479 | 27.087 | 120.012 | 21.413 | 1.00 | 80.06 | 6 |
| ATOM | 7256 | CG1 | VAL | C | 479 | 26.146 | 119.259 | 22.325 | 1.00 | 80.61 | 6 |
| ATOM | 7257 | CG2 | VAL | C | 479 | 26.686 | 119.835 | 19.963 | 1.00 | 80.04 | 6 |
| ATOM | 7258 | C | VAL | C | 479 | 28.484 | 122.036 | 21.507 | 1.00 | 67.45 | 6 |
| ATOM | 7259 | O | VAL | C | 479 | 29.404 | 121.748 | 22.266 | 1.00 | 67.56 | 8 |
| ATOM | 7260 | N | THR | C | 480 | 28.633 | 122.814 | 20.439 | 1.00 | 38.12 | 7 |
| ATOM | 7261 | CA | THR | C | 480 | 29.930 | 123.373 | 20.091 | 1.00 | 37.95 | 6 |
| ATOM | 7262 | CB | THR | C | 480 | 30.187 | 124.735 | 20.803 | 1.00 | 59.69 | 6 |
| ATOM | 7263 | OG1 | THR | C | 480 | 31.528 | 125.179 | 20.545 | 1.00 | 60.79 | 8 |
| ATOM | 7264 | CG2 | THR | C | 480 | 30.001 | 124.605 | 22.300 | 1.00 | 59.28 | 6 |
| ATOM | 7265 | C | THR | C | 480 | 31.027 | 122.407 | 20.495 | 1.00 | 38.63 | 6 |
| ATOM | 7266 | O | THR | C | 480 | 31.440 | 122.378 | 21.653 | 1.00 | 37.92 | 8 |
| ATOM | 7267 | N | GLU | C | 481 | 31.485 | 121.601 | 19.546 | 1.00 | 70.47 | 7 |
| ATOM | 7268 | CA | GLU | C | 481 | 32.553 | 120.649 | 19.819 | 1.00 | 72.47 | 6 |
| ATOM | 7269 | CB | GLU | C | 481 | 33.082 | 120.056 | 18.512 | 1.00 | 208.87 | 6 |
| ATOM | 7270 | CG | GLU | C | 481 | 32.010 | 119.706 | 17.478 | 1.00 | 208.87 | 6 |
| ATOM | 7271 | CD | GLU | C | 481 | 31.548 | 120.907 | 16.661 | 1.00 | 208.87 | 6 |
| ATOM | 7272 | OE1 | GLU | C | 481 | 30.845 | 121.784 | 17.209 | 1.00 | 208.87 | 8 |
| ATOM | 7273 | OE2 | GLU | C | 481 | 31.893 | 120.978 | 15.461 | 1.00 | 208.87 | 8 |
| ATOM | 7274 | C | GLU | C | 481 | 33.655 | 121.449 | 20.506 | 1.00 | 71.91 | 6 |
| ATOM | 7275 | O | GLU | C | 481 | 34.351 | 122.237 | 19.868 | 1.00 | 72.01 | 8 |
| ATOM | 7276 | N | ALA | C | 482 | 33.796 | 121.258 | 21.812 | 1.00 | 73.53 | 7 |
| ATOM | 7277 | CA | ALA | C | 482 | 34.796 | 121.982 | 22.580 | 1.00 | 74.20 | 6 |
| ATOM | 7278 | CB | ALA | C | 482 | 34.829 | 121.473 | 24.006 | 1.00 | 70.38 | 6 |
| ATOM | 7279 | C | ALA | C | 482 | 36.173 | 121.870 | 21.959 | 1.00 | 75.30 | 6 |
| ATOM | 7280 | O | ALA | C | 482 | 36.348 | 121.224 | 20.929 | 1.00 | 75.99 | 8 |
| ATOM | 7281 | N | ALA | C | 483 | 37.144 | 122.521 | 22.593 | 1.00 | 132.12 | 7 |
| ATOM | 7282 | CA | ALA | C | 483 | 38.531 | 122.523 | 22.136 | 1.00 | 132.18 | 6 |
| ATOM | 7283 | CB | ALA | C | 483 | 38.787 | 123.725 | 21.228 | 1.00 | 145.75 | 6 |
| ATOM | 7284 | C | ALA | C | 483 | 39.430 | 122.585 | 23.366 | 1.00 | 131.50 | 6 |
| ATOM | 7285 | O | ALA | C | 483 | 39.509 | 123.609 | 24.032 | 1.00 | 131.95 | 8 |
| ATOM | 7286 | N | VAL | C | 484 | 40.106 | 121.477 | 23.645 | 1.00 | 42.28 | 7 |
| ATOM | 7287 | CA | VAL | C | 484 | 40.988 | 121.318 | 24.801 | 1.00 | 42.41 | 6 |
| ATOM | 7288 | CB | VAL | C | 484 | 42.096 | 120.318 | 24.498 | 1.00 | 164.56 | 6 |
| ATOM | 7289 | CG1 | VAL | C | 484 | 42.856 | 120.000 | 25.774 | 1.00 | 165.45 | 6 |
| ATOM | 7290 | CG2 | VAL | C | 484 | 41.492 | 119.061 | 23.901 | 1.00 | 165.59 | 6 |
| ATOM | 7291 | C | VAL | C | 484 | 41.615 | 122.563 | 25.397 | 1.00 | 41.56 | 6 |
| ATOM | 7292 | O | VAL | C | 484 | 41.859 | 122.607 | 26.597 | 1.00 | 42.17 | 8 |
| ATOM | 7293 | N | ALA | C | 485 | 41.913 | 123.558 | 24.574 | 1.00 | 71.27 | 7 |
| ATOM | 7294 | CA | ALA | C | 485 | 42.455 | 124.804 | 25.097 | 1.00 | 70.95 | 6 |
| ATOM | 7295 | CB | ALA | C | 485 | 43.592 | 125.307 | 24.224 | 1.00 | 13.87 | 6 |
| ATOM | 7296 | C | ALA | C | 485 | 41.266 | 125.766 | 25.064 | 1.00 | 71.12 | 6 |
| ATOM | 7297 | O | ALA | C | 485 | 41.348 | 126.883 | 24.565 | 1.00 | 71.61 | 8 |
| ATOM | 7298 | N | MET | C | 486 | 40.143 | 125.290 | 25.587 | 1.00 | 78.07 | 7 |
| ATOM | 7299 | CA | MET | C | 486 | 38.918 | 126.067 | 25.625 | 1.00 | 77.99 | 6 |
| ATOM | 7300 | CB | MET | C | 486 | 37.701 | 125.149 | 25.473 | 1.00 | 78.50 | 6 |
| ATOM | 7301 | CG | MET | C | 486 | 36.382 | 125.870 | 25.255 | 1.00 | 78.76 | 6 |
| ATOM | 7302 | SD | MET | C | 486 | 35.061 | 124.774 | 24.651 | 1.00 | 77.29 | 16 |
| ATOM | 7303 | CE | MET | C | 486 | 35.149 | 125.096 | 22.898 | 1.00 | 77.76 | 6 |
| ATOM | 7304 | C | MET | C | 486 | 38.860 | 126.791 | 26.956 | 1.00 | 78.61 | 6 |
| ATOM | 7305 | O | MET | C | 486 | 39.085 | 126.197 | 28.017 | 1.00 | 78.63 | 8 |
| ATOM | 7306 | N | ALA | C | 487 | 38.568 | 128.086 | 26.872 | 1.00 | 157.96 | 7 |
| ATOM | 7307 | CA | ALA | C | 487 | 38.464 | 128.966 | 28.027 | 1.00 | 157.73 | 6 |
| ATOM | 7308 | CB | ALA | C | 487 | 39.405 | 130.159 | 27.856 | 1.00 | 13.87 | 6 |
| ATOM | 7309 | C | ALA | C | 487 | 37.030 | 129.460 | 28.164 | 1.00 | 157.88 | 6 |
| ATOM | 7310 | O | ALA | C | 487 | 36.659 | 130.007 | 29.202 | 1.00 | 158.59 | 8 |
| ATOM | 7311 | N | ALA | C | 488 | 36.238 | 129.255 | 27.109 | 1.00 | 193.55 | 7 |
| ATOM | 7312 | CA | ALA | C | 488 | 34.841 | 129.689 | 27.065 | 1.00 | 194.23 | 6 |
| ATOM | 7313 | CB | ALA | C | 488 | 33.970 | 128.658 | 26.290 | 1.00 | 20.13 | 6 |
| ATOM | 7314 | C | ALA | C | 488 | 34.273 | 129.936 | 28.462 | 1.00 | 194.39 | 6 |
| ATOM | 7315 | O | ALA | C | 488 | 33.604 | 129.083 | 29.042 | 1.00 | 195.34 | 8 |
| ATOM | 7316 | N | ALA | C | 489 | 34.579 | 131.124 | 28.981 | 1.00 | 108.29 | 7 |
| ATOM | 7317 | CA | ALA | C | 489 | 34.156 | 131.609 | 30.292 | 1.00 | 107.01 | 6 |
| ATOM | 7318 | CB | ALA | C | 489 | 33.173 | 132.745 | 30.111 | 1.00 | 21.82 | 6 |
| ATOM | 7319 | C | ALA | C | 489 | 33.582 | 130.606 | 31.281 | 1.00 | 106.31 | 6 |
| ATOM | 7320 | O | ALA | C | 489 | 32.551 | 130.878 | 31.894 | 1.00 | 106.68 | 8 |
| ATOM | 7321 | N | ALA | C | 490 | 34.243 | 129.463 | 31.447 | 1.00 | 94.59 | 7 |
| ATOM | 7322 | CA | ALA | C | 490 | 33.788 | 128.438 | 32.389 | 1.00 | 93.90 | 6 |
| ATOM | 7323 | CB | ALA | C | 490 | 33.302 | 129.098 | 33.676 | 1.00 | 13.87 | 6 |
| ATOM | 7324 | C | ALA | C | 490 | 32.699 | 127.506 | 31.861 | 1.00 | 93.34 | 6 |
| ATOM | 7325 | O | ALA | C | 490 | 31.927 | 127.859 | 30.972 | 1.00 | 95.08 | 8 |
| ATOM | 7326 | N | GLU | C | 491 | 32.652 | 126.307 | 32.427 | 1.00 | 45.09 | 7 |
| ATOM | 7327 | CA | GLU | C | 491 | 31.659 | 125.310 | 32.067 | 1.00 | 43.70 | 6 |
| ATOM | 7328 | CB | GLU | C | 491 | 32.324 | 124.068 | 31.460 | 1.00 | 101.46 | 6 |
| ATOM | 7329 | CG | GLU | C | 491 | 33.573 | 123.594 | 32.195 | 1.00 | 103.12 | 6 |
| ATOM | 7330 | CD | GLU | C | 491 | 34.161 | 122.314 | 31.612 | 1.00 | 103.44 | 6 |
| ATOM | 7331 | OE1 | GLU | C | 491 | 34.108 | 122.151 | 30.374 | 1.00 | 103.66 | 8 |
| ATOM | 7332 | OE2 | GLU | C | 491 | 34.688 | 121.481 | 32.388 | 1.00 | 102.72 | 8 |
| ATOM | 7333 | C | GLU | C | 491 | 30.978 | 124.972 | 33.381 | 1.00 | 43.30 | 6 |
| ATOM | 7334 | O | GLU | C | 491 | 31.634 | 124.938 | 34.422 | 1.00 | 43.64 | 8 |
| ATOM | 7335 | N | ALA | C | 492 | 29.666 | 124.749 | 33.342 | 1.00 | 48.24 | 7 |
| ATOM | 7336 | CA | ALA | C | 492 | 28.905 | 124.442 | 34.553 | 1.00 | 47.20 | 6 |
| ATOM | 7337 | CB | ALA | C | 492 | 27.734 | 125.398 | 34.685 | 1.00 | 126.11 | 6 |
| ATOM | 7338 | C | ALA | C | 492 | 28.410 | 123.009 | 34.563 | 1.00 | 47.18 | 6 |
| ATOM | 7339 | O | ALA | C | 492 | 27.500 | 122.647 | 33.813 | 1.00 | 47.47 | 8 |
| ATOM | 7340 | N | ARG | C | 493 | 29.016 | 122.210 | 35.441 | 1.00 | 93.61 | 7 |
| ATOM | 7341 | CA | ARG | C | 493 | 28.694 | 120.795 | 35.582 | 1.00 | 93.11 | 6 |
| ATOM | 7342 | CB | ARG | C | 493 | 29.323 | 120.229 | 36.863 | 1.00 | 61.11 | 6 |
| ATOM | 7343 | CG | ARG | C | 493 | 30.851 | 120.303 | 36.838 | 1.00 | 58.90 | 6 |
| ATOM | 7344 | CD | ARG | C | 493 | 31.522 | 119.616 | 38.026 | 1.00 | 57.23 | 6 |
| ATOM | 7345 | NE | ARG | C | 493 | 32.980 | 119.785 | 37.999 | 1.00 | 54.51 | 7 |
| ATOM | 7346 | CZ | ARG | C | 493 | 33.820 | 119.257 | 38.887 | 1.00 | 52.88 | 6 |
| ATOM | 7347 | NH1 | ARG | C | 493 | 33.363 | 118.521 | 39.885 | 1.00 | 51.83 | 7 |
| ATOM | 7348 | NH2 | ARG | C | 493 | 35.123 | 119.456 | 38.772 | 1.00 | 51.80 | 7 |
| ATOM | 7349 | C | ARG | C | 493 | 27.208 | 120.505 | 35.547 | 1.00 | 93.57 | 6 |
| ATOM | 7350 | O | ARG | C | 493 | 26.394 | 121.260 | 36.077 | 1.00 | 93.81 | 8 |
| ATOM | 7351 | N | TYR | C | 494 | 26.880 | 119.396 | 34.895 | 1.00 | 57.29 | 7 |
| ATOM | 7352 | CA | TYR | C | 494 | 25.515 | 118.926 | 34.713 | 1.00 | 58.47 | 6 |
| ATOM | 7353 | CB | TYR | C | 494 | 25.564 | 117.505 | 34.177 | 1.00 | 80.31 | 6 |
| ATOM | 7354 | CG | TYR | C | 494 | 24.221 | 116.860 | 33.938 | 1.00 | 81.49 | 6 |
| ATOM | 7355 | CD1 | TYR | C | 494 | 23.343 | 117.368 | 32.982 | 1.00 | 81.70 | 6 |
| ATOM | 7356 | CE1 | TYR | C | 494 | 22.156 | 116.705 | 32.680 | 1.00 | 82.02 | 6 |
| ATOM | 7357 | CD2 | TYR | C | 494 | 23.870 | 115.677 | 34.599 | 1.00 | 82.19 | 6 |
| ATOM | 7358 | CE2 | TYR | C | 494 | 22.689 | 115.009 | 34.304 | 1.00 | 82.33 | 6 |
| ATOM | 7359 | CZ | TYR | C | 494 | 21.844 | 115.526 | 33.342 | 1.00 | 82.56 | 6 |
| ATOM | 7360 | OH | TYR | C | 494 | 20.709 | 114.845 | 33.016 | 1.00 | 83.98 | 8 |
| ATOM | 7361 | C | TYR | C | 494 | 24.619 | 118.975 | 35.950 | 1.00 | 59.88 | 6 |
| ATOM | 7362 | O | TYR | C | 494 | 25.062 | 118.690 | 37.073 | 1.00 | 60.16 | 8 |
| ATOM | 7363 | N | THR | C | 495 | 23.355 | 119.334 | 35.706 | 1.00 | 111.91 | 7 |
| ATOM | 7364 | CA | THR | C | 495 | 22.294 | 119.446 | 36.711 | 1.00 | 112.57 | 6 |
| ATOM | 7365 | CB | THR | C | 495 | 22.618 | 118.610 | 37.978 | 1.00 | 98.93 | 6 |
| ATOM | 7366 | OG1 | THR | C | 495 | 23.125 | 117.329 | 37.586 | 1.00 | 98.83 | 8 |
| ATOM | 7367 | CG2 | THR | C | 495 | 21.362 | 118.385 | 38.807 | 1.00 | 99.57 | 6 |
| ATOM | 7368 | C | THR | C | 495 | 21.986 | 120.904 | 37.107 | 1.00 | 113.56 | 6 |
| ATOM | 7369 | O | THR | C | 495 | 22.722 | 121.829 | 36.743 | 1.00 | 113.52 | 8 |
| ATOM | 7370 | N | ALA | C | 496 | 20.884 | 121.075 | 37.845 | 1.00 | 85.32 | 7 |
| ATOM | 7371 | CA | ALA | C | 496 | 20.354 | 122.358 | 38.333 | 1.00 | 85.17 | 6 |
| ATOM | 7372 | CB | ALA | C | 496 | 21.405 | 123.443 | 38.301 | 1.00 | 64.23 | 6 |
| ATOM | 7373 | C | ALA | C | 496 | 19.213 | 122.722 | 37.408 | 1.00 | 85.77 | 6 |
| ATOM | 7374 | O | ALA | C | 496 | 18.605 | 123.780 | 37.529 | 1.00 | 86.30 | 8 |
| ATOM | 7375 | N | ALA | C | 497 | 18.938 | 121.810 | 36.483 | 1.00 | 105.36 | 7 |
| ATOM | 7376 | CA | ALA | C | 497 | 17.895 | 121.962 | 35.481 | 1.00 | 106.23 | 6 |
| ATOM | 7377 | CB | ALA | C | 497 | 17.616 | 120.611 | 34.831 | 1.00 | 140.99 | 6 |
| ATOM | 7378 | C | ALA | C | 497 | 16.589 | 122.579 | 35.969 | 1.00 | 106.61 | 6 |
| ATOM | 7379 | O | ALA | C | 497 | 16.060 | 122.223 | 37.024 | 1.00 | 106.73 | 8 |
| ATOM | 7380 | N | ALA | C | 498 | 16.082 | 123.516 | 35.178 | 1.00 | 105.43 | 7 |
| ATOM | 7381 | CA | ALA | C | 498 | 14.827 | 124.200 | 35.455 | 1.00 | 106.36 | 6 |
| ATOM | 7382 | CB | ALA | C | 498 | 15.074 | 125.547 | 36.146 | 1.00 | 32.90 | 6 |
| ATOM | 7383 | C | ALA | C | 498 | 14.207 | 124.407 | 34.082 | 1.00 | 106.88 | 6 |
| ATOM | 7384 | O | ALA | C | 498 | 14.027 | 125.539 | 33.622 | 1.00 | 107.64 | 8 |
| ATOM | 7385 | N | ALA | C | 499 | 13.907 | 123.283 | 33.433 | 1.00 | 111.52 | 7 |
| ATOM | 7386 | CA | ALA | C | 499 | 13.316 | 123.259 | 32.101 | 1.00 | 111.25 | 6 |
| ATOM | 7387 | CB | ALA | C | 499 | 12.043 | 124.108 | 32.060 | 1.00 | 120.72 | 6 |
| ATOM | 7388 | C | ALA | C | 499 | 14.327 | 123.779 | 31.097 | 1.00 | 110.44 | 6 |
| ATOM | 7389 | O | ALA | C | 499 | 14.990 | 123.010 | 30.403 | 1.00 | 110.80 | 8 |
| ATOM | 7390 | N | ASN | C | 500 | 14.451 | 125.095 | 31.036 | 1.00 | 43.61 | 7 |
| ATOM | 7391 | CA | ASN | C | 500 | 15.379 | 125.717 | 30.115 | 1.00 | 43.08 | 6 |
| ATOM | 7392 | CB | ASN | C | 500 | 14.598 | 126.438 | 29.010 | 1.00 | 114.29 | 6 |
| ATOM | 7393 | CG | ASN | C | 500 | 13.934 | 125.480 | 28.037 | 1.00 | 115.71 | 6 |
| ATOM | 7394 | OD1 | ASN | C | 500 | 14.560 | 125.013 | 27.087 | 1.00 | 117.56 | 8 |
| ATOM | 7395 | ND2 | ASN | C | 500 | 12.662 | 125.179 | 28.274 | 1.00 | 115.42 | 7 |
| ATOM | 7396 | C | ASN | C | 500 | 16.288 | 126.710 | 30.834 | 1.00 | 42.38 | 6 |
| ATOM | 7397 | O | ASN | C | 500 | 17.383 | 127.003 | 30.362 | 1.00 | 41.72 | 8 |
| ATOM | 7398 | N | THR | C | 501 | 15.850 | 127.206 | 31.988 | 1.00 | 58.14 | 7 |
| ATOM | 7399 | CA | THR | C | 501 | 16.627 | 128.206 | 32.725 | 1.00 | 58.92 | 6 |
| ATOM | 7400 | CB | THR | C | 501 | 15.698 | 129.360 | 33.207 | 1.00 | 174.72 | 6 |
| ATOM | 7401 | OG1 | THR | C | 501 | 14.803 | 129.737 | 32.152 | 1.00 | 176.53 | 8 |
| ATOM | 7402 | CG2 | THR | C | 501 | 16.517 | 130.580 | 33.606 | 1.00 | 175.70 | 6 |
| ATOM | 7403 | C | THR | C | 501 | 17.426 | 127.713 | 33.940 | 1.00 | 57.69 | 6 |
| ATOM | 7404 | O | THR | C | 501 | 18.193 | 128.476 | 34.537 | 1.00 | 56.54 | 8 |
| ATOM | 7405 | N | PRO | C | 502 | 17.325 | 126.418 | 34.267 | 1.00 | 51.85 | 7 |
| ATOM | 7406 | CD | PRO | C | 502 | 17.400 | 125.367 | 33.236 | 1.00 | 194.50 | 6 |
| ATOM | 7407 | CA | PRO | C | 502 | 18.070 | 125.961 | 35.434 | 1.00 | 51.22 | 6 |
| ATOM | 7408 | CB | PRO | C | 502 | 19.315 | 125.371 | 34.802 | 1.00 | 194.09 | 6 |
| ATOM | 7409 | CG | PRO | C | 502 | 18.696 | 124.601 | 33.620 | 1.00 | 194.75 | 6 |
| ATOM | 7410 | C | PRO | C | 502 | 18.339 | 127.023 | 36.485 | 1.00 | 50.18 | 6 |
| ATOM | 7411 | O | PRO | C | 502 | 19.440 | 127.549 | 36.603 | 1.00 | 49.51 | 8 |
| ATOM | 7412 | N | ALA | C | 503 | 17.302 | 127.346 | 37.242 | 1.00 | 83.31 | 7 |
| ATOM | 7413 | CA | ALA | C | 503 | 17.443 | 128.326 | 38.299 | 1.00 | 82.70 | 6 |
| ATOM | 7414 | CB | ALA | C | 503 | 16.079 | 128.700 | 38.858 | 1.00 | 157.97 | 6 |
| ATOM | 7415 | C | ALA | C | 503 | 18.287 | 127.646 | 39.366 | 1.00 | 81.70 | 6 |
| ATOM | 7416 | O | ALA | C | 503 | 17.863 | 126.653 | 39.968 | 1.00 | 80.59 | 8 |
| ATOM | 7417 | N | ALA | C | 504 | 19.486 | 128.176 | 39.587 | 1.00 | 61.52 | 7 |
| ATOM | 7418 | CA | ALA | C | 504 | 20.387 | 127.599 | 40.565 | 1.00 | 61.35 | 6 |
| ATOM | 7419 | CB | ALA | C | 504 | 21.666 | 127.154 | 39.877 | 1.00 | 150.60 | 6 |
| ATOM | 7420 | C | ALA | C | 504 | 20.723 | 128.532 | 41.710 | 1.00 | 60.28 | 6 |
| ATOM | 7421 | O | ALA | C | 504 | 20.310 | 128.309 | 42.847 | 1.00 | 59.15 | 8 |
| ATOM | 7422 | N | GLY | C | 505 | 21.464 | 129.586 | 41.391 | 1.00 | 89.86 | 7 |
| ATOM | 7423 | CA | GLY | C | 505 | 21.901 | 130.528 | 42.403 | 1.00 | 90.42 | 6 |
| ATOM | 7424 | C | GLY | C | 505 | 23.341 | 130.117 | 42.636 | 1.00 | 90.60 | 6 |
| ATOM | 7425 | O | GLY | C | 505 | 24.243 | 130.940 | 42.799 | 1.00 | 91.07 | 8 |
| ATOM | 7426 | N | ASP | C | 506 | 23.544 | 128.807 | 42.646 | 1.00 | 82.76 | 7 |
| ATOM | 7427 | CA | ASP | C | 506 | 24.864 | 128.229 | 42.806 | 1.00 | 81.90 | 6 |
| ATOM | 7428 | CB | ASP | C | 506 | 25.011 | 127.538 | 44.159 | 1.00 | 67.53 | 6 |
| ATOM | 7429 | CG | ASP | C | 506 | 25.609 | 128.442 | 45.207 | 1.00 | 66.91 | 6 |
| ATOM | 7430 | OD1 | ASP | C | 506 | 26.570 | 129.162 | 44.876 | 1.00 | 66.91 | 8 |
| ATOM | 7431 | OD2 | ASP | C | 506 | 25.132 | 128.427 | 46.361 | 1.00 | 66.05 | 8 |
| ATOM | 7432 | C | ASP | C | 506 | 25.026 | 127.210 | 41.694 | 1.00 | 81.36 | 6 |
| ATOM | 7433 | O | ASP | C | 506 | 24.542 | 126.082 | 41.796 | 1.00 | 81.62 | 8 |
| ATOM | 7434 | N | ARG | C | 507 | 25.683 | 127.624 | 40.617 | 1.00 | 58.94 | 7 |
| ATOM | 7435 | CA | ARG | C | 507 | 25.907 | 126.735 | 39.493 | 1.00 | 57.88 | 6 |
| ATOM | 7436 | CB | ARG | C | 507 | 25.913 | 127.528 | 38.176 | 1.00 | 68.18 | 6 |
| ATOM | 7437 | CG | ARG | C | 507 | 24.603 | 128.259 | 37.838 | 1.00 | 67.43 | 6 |
| ATOM | 7438 | CD | ARG | C | 507 | 23.667 | 127.447 | 36.925 | 1.00 | 66.82 | 6 |
| ATOM | 7439 | NE | ARG | C | 507 | 22.497 | 128.224 | 36.484 | 1.00 | 66.76 | 7 |
| ATOM | 7440 | CZ | ARG | C | 507 | 21.711 | 127.902 | 35.452 | 1.00 | 66.55 | 6 |
| ATOM | 7441 | NH1 | ARG | C | 507 | 21.953 | 126.812 | 34.740 | 1.00 | 67.25 | 7 |
| ATOM | 7442 | NH2 | ARG | C | 507 | 20.686 | 128.679 | 35.114 | 1.00 | 66.00 | 7 |
| ATOM | 7443 | C | ARG | C | 507 | 27.267 | 126.094 | 39.726 | 1.00 | 57.63 | 6 |
| ATOM | 7444 | O | ARG | C | 507 | 28.287 | 126.763 | 39.609 | 1.00 | 57.50 | 8 |
| ATOM | 7445 | N | ILE | C | 508 | 27.276 | 124.810 | 40.084 | 1.00 | 61.89 | 7 |
| ATOM | 7446 | CA | ILE | C | 508 | 28.528 | 124.088 | 40.314 | 1.00 | 62.24 | 6 |
| ATOM | 7447 | CB | ILE | C | 508 | 28.281 | 122.634 | 40.777 | 1.00 | 88.29 | 6 |
| ATOM | 7448 | CG2 | ILE | C | 508 | 29.423 | 122.182 | 41.678 | 1.00 | 87.84 | 6 |
| ATOM | 7449 | CG1 | ILE | C | 508 | 26.928 | 122.516 | 41.492 | 1.00 | 89.46 | 6 |
| ATOM | 7450 | CD1 | ILE | C | 508 | 26.782 | 123.365 | 42.747 | 1.00 | 92.22 | 6 |
| ATOM | 7451 | C | ILE | C | 508 | 29.201 | 124.047 | 38.950 | 1.00 | 62.15 | 6 |
| ATOM | 7452 | O | ILE | C | 508 | 28.743 | 123.347 | 38.046 | 1.00 | 62.11 | 8 |
| ATOM | 7453 | N | ALA | C | 509 | 30.287 | 124.791 | 38.789 | 1.00 | 52.75 | 7 |
| ATOM | 7454 | CA | ALA | C | 509 | 30.936 | 124.835 | 37.490 | 1.00 | 53.21 | 6 |
| ATOM | 7455 | CB | ALA | C | 509 | 30.547 | 126.118 | 36.771 | 1.00 | 107.64 | 6 |
| ATOM | 7456 | C | ALA | C | 509 | 32.441 | 124.699 | 37.515 | 1.00 | 53.71 | 6 |
| ATOM | 7457 | O | ALA | C | 509 | 33.103 | 124.973 | 38.522 | 1.00 | 53.78 | 8 |
| ATOM | 7458 | N | THR | C | 510 | 32.963 | 124.276 | 36.370 | 1.00 | 30.63 | 7 |
| ATOM | 7459 | CA | THR | C | 510 | 34.387 | 124.060 | 36.170 | 1.00 | 32.20 | 6 |
| ATOM | 7460 | CB | THR | C | 510 | 34.670 | 122.720 | 35.398 | 1.00 | 79.27 | 6 |
| ATOM | 7461 | OG1 | THR | C | 510 | 33.839 | 121.663 | 35.897 | 1.00 | 79.86 | 8 |
| ATOM | 7462 | CG2 | THR | C | 510 | 36.116 | 122.308 | 35.568 | 1.00 | 80.57 | 6 |
| ATOM | 7463 | C | THR | C | 510 | 34.898 | 125.202 | 35.304 | 1.00 | 33.62 | 6 |
| ATOM | 7464 | O | THR | C | 510 | 34.344 | 125.480 | 34.241 | 1.00 | 33.24 | 8 |
| ATOM | 7465 | N | ASP | C | 511 | 35.938 | 125.879 | 35.769 | 1.00 | 82.32 | 7 |
| ATOM | 7466 | CA | ASP | C | 511 | 36.535 | 126.947 | 34.991 | 1.00 | 85.09 | 6 |
| ATOM | 7467 | CB | ASP | C | 511 | 36.118 | 128.327 | 35.514 | 1.00 | 169.52 | 6 |
| ATOM | 7468 | CG | ASP | C | 511 | 36.831 | 128.715 | 36.783 | 1.00 | 172.77 | 6 |
| ATOM | 7469 | OD1 | ASP | C | 511 | 36.832 | 127.907 | 37.733 | 1.00 | 174.27 | 8 |
| ATOM | 7470 | OD2 | ASP | C | 511 | 37.384 | 129.834 | 36.830 | 1.00 | 173.99 | 8 |
| ATOM | 7471 | C | ASP | C | 511 | 38.033 | 126.725 | 35.116 | 1.00 | 86.28 | 6 |
| ATOM | 7472 | O | ASP | C | 511 | 38.574 | 126.629 | 36.218 | 1.00 | 86.46 | 8 |
| ATOM | 7473 | N | ARG | C | 512 | 38.688 | 126.604 | 33.967 | 1.00 | 86.90 | 7 |
| ATOM | 7474 | CA | ARG | C | 512 | 40.122 | 126.357 | 33.889 | 1.00 | 87.82 | 6 |
| ATOM | 7475 | CB | ARG | C | 512 | 40.927 | 127.487 | 34.541 | 1.00 | 96.06 | 6 |
| ATOM | 7476 | CG | ARG | C | 512 | 42.398 | 127.386 | 34.195 | 1.00 | 96.74 | 6 |
| ATOM | 7477 | CD | ARG | C | 512 | 43.316 | 128.011 | 35.217 | 1.00 | 96.67 | 6 |
| ATOM | 7478 | NE | ARG | C | 512 | 44.680 | 127.551 | 34.979 | 1.00 | 96.37 | 7 |
| ATOM | 7479 | CZ | ARG | C | 512 | 45.720 | 127.823 | 35.757 | 1.00 | 96.51 | 6 |
| ATOM | 7480 | NH1 | ARG | C | 512 | 45.570 | 128.568 | 36.843 | 1.00 | 97.43 | 7 |
| ATOM | 7481 | NH2 | ARG | C | 512 | 46.912 | 127.332 | 35.451 | 1.00 | 95.66 | 7 |
| ATOM | 7482 | C | ARG | C | 512 | 40.482 | 125.039 | 34.561 | 1.00 | 88.16 | 6 |
| ATOM | 7483 | O | ARG | C | 512 | 40.539 | 123.993 | 33.915 | 1.00 | 88.76 | 8 |
| ATOM | 7484 | N | ALA | C | 513 | 40.722 | 125.096 | 35.865 | 1.00 | 141.03 | 7 |
| ATOM | 7485 | CA | ALA | C | 513 | 41.086 | 123.908 | 36.616 | 1.00 | 141.99 | 6 |
| ATOM | 7486 | CB | ALA | C | 513 | 42.563 | 123.963 | 36.978 | 1.00 | 124.59 | 6 |
| ATOM | 7487 | C | ALA | C | 513 | 40.242 | 123.771 | 37.874 | 1.00 | 141.81 | 6 |
| ATOM | 7488 | O | ALA | C | 513 | 39.400 | 122.873 | 37.972 | 1.00 | 141.21 | 8 |
| ATOM | 7489 | N | ALA | C | 514 | 40.478 | 124.663 | 38.834 | 1.00 | 56.61 | 7 |
| ATOM | 7490 | CA | ALA | C | 514 | 39.745 | 124.653 | 40.094 | 1.00 | 56.03 | 6 |
| ATOM | 7491 | CB | ALA | C | 514 | 39.916 | 125.989 | 40.800 | 1.00 | 86.46 | 6 |
| ATOM | 7492 | C | ALA | C | 514 | 38.260 | 124.367 | 39.871 | 1.00 | 56.39 | 6 |
| ATOM | 7493 | O | ALA | C | 514 | 37.675 | 124.770 | 38.859 | 1.00 | 56.05 | 8 |
| ATOM | 7494 | N | ALA | C | 515 | 37.662 | 123.655 | 40.819 | 1.00 | 79.23 | 7 |
| ATOM | 7495 | CA | ALA | C | 515 | 36.248 | 123.320 | 40.753 | 1.00 | 80.00 | 6 |
| ATOM | 7496 | CB | ALA | C | 515 | 35.969 | 122.106 | 41.635 | 1.00 | 13.87 | 6 |
| ATOM | 7497 | C | ALA | C | 515 | 35.475 | 124.530 | 41.263 | 1.00 | 80.71 | 6 |
| ATOM | 7498 | O | ALA | C | 515 | 34.437 | 124.386 | 41.905 | 1.00 | 81.84 | 8 |
| ATOM | 7499 | N | ARG | C | 516 | 35.989 | 125.720 | 40.960 | 1.00 | 51.67 | 7 |
| ATOM | 7500 | CA | ARG | C | 516 | 35.402 | 126.977 | 41.414 | 1.00 | 52.83 | 6 |
| ATOM | 7501 | CB | ARG | C | 516 | 35.864 | 128.127 | 40.508 | 1.00 | 184.59 | 6 |
| ATOM | 7502 | CG | ARG | C | 516 | 37.372 | 128.365 | 40.645 | 1.00 | 186.25 | 6 |
| ATOM | 7503 | CD | ARG | C | 516 | 37.860 | 129.694 | 40.076 | 1.00 | 188.19 | 6 |
| ATOM | 7504 | NE | ARG | C | 516 | 39.271 | 129.915 | 40.405 | 1.00 | 188.67 | 7 |
| ATOM | 7505 | CZ | ARG | C | 516 | 39.960 | 131.008 | 40.087 | 1.00 | 188.92 | 6 |
| ATOM | 7506 | NH1 | ARG | C | 516 | 39.377 | 131.999 | 39.425 | 1.00 | 188.66 | 7 |
| ATOM | 7507 | NH2 | ARG | C | 516 | 41.237 | 131.113 | 40.435 | 1.00 | 187.95 | 7 |
| ATOM | 7508 | C | ARG | C | 516 | 33.892 | 127.000 | 41.620 | 1.00 | 53.89 | 6 |
| ATOM | 7509 | O | ARG | C | 516 | 33.109 | 126.812 | 40.684 | 1.00 | 53.00 | 8 |
| ATOM | 7510 | N | ARG | C | 517 | 33.538 | 127.218 | 42.894 | 1.00 | 97.04 | 7 |
| ATOM | 7511 | CA | ARG | C | 517 | 32.179 | 127.293 | 43.460 | 1.00 | 99.09 | 6 |
| ATOM | 7512 | CB | ARG | C | 517 | 32.102 | 128.467 | 44.452 | 1.00 | 153.60 | 6 |
| ATOM | 7513 | CG | ARG | C | 517 | 30.689 | 128.839 | 44.899 | 1.00 | 154.88 | 6 |
| ATOM | 7514 | CD | ARG | C | 517 | 30.408 | 128.483 | 46.359 | 1.00 | 156.22 | 6 |
| ATOM | 7515 | NE | ARG | C | 517 | 31.052 | 129.399 | 47.300 | 1.00 | 156.32 | 7 |
| ATOM | 7516 | CZ | ARG | C | 517 | 30.759 | 129.473 | 48.598 | 1.00 | 156.14 | 6 |
| ATOM | 7517 | NH1 | ARG | C | 517 | 29.827 | 128.685 | 49.126 | 1.00 | 155.63 | 7 |
| ATOM | 7518 | NH2 | ARG | C | 517 | 31.397 | 130.338 | 49.373 | 1.00 | 156.15 | 7 |
| ATOM | 7519 | C | ARG | C | 517 | 31.014 | 127.388 | 42.501 | 1.00 | 99.92 | 6 |
| ATOM | 7520 | O | ARG | C | 517 | 30.392 | 126.388 | 42.151 | 1.00 | 99.19 | 8 |
| ATOM | 7521 | N | ALA | C | 518 | 30.710 | 128.610 | 42.096 | 1.00 | 151.09 | 7 |
| ATOM | 7522 | CA | ALA | C | 518 | 29.613 | 128.841 | 41.187 | 1.00 | 154.07 | 6 |
| ATOM | 7523 | CB | ALA | C | 518 | 28.288 | 128.715 | 41.932 | 1.00 | 16.95 | 6 |
| ATOM | 7524 | C | ALA | C | 518 | 29.757 | 130.227 | 40.597 | 1.00 | 156.60 | 6 |
| ATOM | 7525 | O | ALA | C | 518 | 29.198 | 131.186 | 41.124 | 1.00 | 158.28 | 8 |
| ATOM | 7526 | N | GLY | C | 519 | 30.521 | 130.336 | 39.513 | 1.00 | 153.30 | 7 |
| ATOM | 7527 | CA | GLY | C | 519 | 30.706 | 131.628 | 38.875 | 1.00 | 155.29 | 6 |
| ATOM | 7528 | C | GLY | C | 519 | 29.359 | 132.307 | 38.758 | 1.00 | 156.47 | 6 |
| ATOM | 7529 | O | GLY | C | 519 | 29.251 | 133.535 | 38.738 | 1.00 | 156.88 | 8 |
| ATOM | 7530 | N | ALA | C | 520 | 28.327 | 131.473 | 38.677 | 1.00 | 125.80 | 7 |
| ATOM | 7531 | CA | ALA | C | 520 | 26.946 | 131.917 | 38.582 | 1.00 | 127.15 | 6 |
| ATOM | 7532 | CB | ALA | C | 520 | 26.489 | 132.455 | 39.933 | 1.00 | 108.64 | 6 |
| ATOM | 7533 | C | ALA | C | 520 | 26.659 | 132.950 | 37.494 | 1.00 | 128.57 | 6 |
| ATOM | 7534 | O | ALA | C | 520 | 25.733 | 133.753 | 37.644 | 1.00 | 129.26 | 8 |
| ATOM | 7535 | N | PRO | C | 521 | 27.425 | 132.938 | 36.378 | 1.00 | 208.87 | 7 |
| ATOM | 7536 | CD | PRO | C | 521 | 28.431 | 131.966 | 35.911 | 1.00 | 138.40 | 6 |
| ATOM | 7537 | CA | PRO | C | 521 | 27.154 | 133.928 | 35.330 | 1.00 | 208.87 | 6 |
| ATOM | 7538 | CB | PRO | C | 521 | 27.887 | 133.359 | 34.120 | 1.00 | 137.39 | 6 |
| ATOM | 7539 | CG | PRO | C | 521 | 29.061 | 132.689 | 34.740 | 1.00 | 137.82 | 6 |
| ATOM | 7540 | C | PRO | C | 521 | 25.657 | 134.027 | 35.098 | 1.00 | 208.87 | 6 |
| ATOM | 7541 | O | PRO | C | 521 | 25.151 | 135.052 | 34.643 | 1.00 | 208.87 | 8 |
| ATOM | 7542 | N | ALA | C | 522 | 24.963 | 132.944 | 35.428 | 1.00 | 126.01 | 7 |
| ATOM | 7543 | CA | ALA | C | 522 | 23.522 | 132.869 | 35.290 | 1.00 | 125.89 | 6 |
| ATOM | 7544 | CB | ALA | C | 522 | 22.857 | 133.341 | 36.580 | 1.00 | 182.72 | 6 |
| ATOM | 7545 | C | ALA | C | 522 | 23.029 | 133.693 | 34.100 | 1.00 | 125.77 | 6 |
| ATOM | 7546 | O | ALA | C | 522 | 22.082 | 134.471 | 34.219 | 1.00 | 125.75 | 8 |
| ATOM | 7547 | N | ALA | C | 523 | 23.689 | 133.530 | 32.954 | 1.00 | 135.59 | 7 |
| ATOM | 7548 | CA | ALA | C | 523 | 23.308 | 134.246 | 31.737 | 1.00 | 135.13 | 6 |
| ATOM | 7549 | CB | ALA | C | 523 | 24.499 | 134.348 | 30.776 | 1.00 | 81.38 | 6 |
| ATOM | 7550 | C | ALA | C | 523 | 22.161 | 133.503 | 31.070 | 1.00 | 135.17 | 6 |
| ATOM | 7551 | O | ALA | C | 523 | 21.694 | 133.912 | 30.009 | 1.00 | 135.05 | 8 |
| ATOM | 7552 | N | ALA | C | 524 | 21.728 | 132.410 | 31.702 | 1.00 | 120.70 | 7 |
| ATOM | 7553 | CA | ALA | C | 524 | 20.631 | 131.582 | 31.208 | 1.00 | 120.84 | 6 |
| ATOM | 7554 | CB | ALA | C | 524 | 19.303 | 132.271 | 31.483 | 1.00 | 194.26 | 6 |
| ATOM | 7555 | C | ALA | C | 524 | 20.788 | 131.299 | 29.713 | 1.00 | 120.68 | 6 |
| ATOM | 7556 | O | ALA | C | 524 | 21.040 | 130.160 | 29.309 | 1.00 | 120.75 | 8 |
| ATOM | 7557 | N | ALA | C | 525 | 20.609 | 132.336 | 28.898 | 1.00 | 141.28 | 7 |
| ATOM | 7558 | CA | ALA | C | 525 | 20.779 | 132.240 | 27.452 | 1.00 | 140.31 | 6 |
| ATOM | 7559 | CB | ALA | C | 525 | 20.321 | 133.532 | 26.791 | 1.00 | 135.97 | 6 |
| ATOM | 7560 | C | ALA | C | 525 | 22.287 | 132.059 | 27.307 | 1.00 | 139.91 | 6 |
| ATOM | 7561 | O | ALA | C | 525 | 23.009 | 132.243 | 28.287 | 1.00 | 139.53 | 8 |
| ATOM | 7562 | N | PRO | C | 526 | 22.788 | 131.771 | 26.089 | 1.00 | 159.42 | 7 |
| ATOM | 7563 | CD | PRO | C | 526 | 22.311 | 132.434 | 24.863 | 1.00 | 208.87 | 6 |
| ATOM | 7564 | CA | PRO | C | 526 | 24.231 | 131.581 | 25.970 | 1.00 | 159.07 | 6 |
| ATOM | 7565 | CB | PRO | C | 526 | 24.677 | 132.935 | 25.461 | 1.00 | 208.87 | 6 |
| ATOM | 7566 | CG | PRO | C | 526 | 23.575 | 133.212 | 24.394 | 1.00 | 208.87 | 6 |
| ATOM | 7567 | C | PRO | C | 526 | 24.881 | 131.136 | 27.273 | 1.00 | 158.57 | 6 |
| ATOM | 7568 | O | PRO | C | 526 | 25.861 | 131.715 | 27.732 | 1.00 | 159.08 | 8 |
| ATOM | 7569 | N | ALA | C | 527 | 24.287 | 130.095 | 27.854 | 1.00 | 119.34 | 7 |
| ATOM | 7570 | CA | ALA | C | 527 | 24.705 | 129.485 | 29.113 | 1.00 | 118.20 | 6 |
| ATOM | 7571 | CB | ALA | C | 527 | 24.608 | 127.986 | 28.990 | 1.00 | 51.31 | 6 |
| ATOM | 7572 | C | ALA | C | 527 | 26.107 | 129.872 | 29.563 | 1.00 | 117.87 | 6 |
| ATOM | 7573 | O | ALA | C | 527 | 27.074 | 129.546 | 28.881 | 1.00 | 118.65 | 8 |
| ATOM | 7574 | N | ALA | C | 528 | 26.206 | 130.545 | 30.714 | 1.00 | 187.90 | 7 |
| ATOM | 7575 | CA | ALA | C | 528 | 27.488 | 130.992 | 31.273 | 1.00 | 186.63 | 6 |
| ATOM | 7576 | CB | ALA | C | 528 | 27.687 | 130.398 | 32.677 | 1.00 | 25.37 | 6 |
| ATOM | 7577 | C | ALA | C | 528 | 28.638 | 130.583 | 30.358 | 1.00 | 186.26 | 6 |
| ATOM | 7578 | O | ALA | C | 528 | 29.355 | 129.626 | 30.646 | 1.00 | 186.75 | 8 |
| ATOM | 7579 | N | ALA | C | 529 | 28.798 | 131.312 | 29.254 | 1.00 | 208.87 | 7 |
| ATOM | 7580 | CA | ALA | C | 529 | 29.829 | 131.018 | 28.259 | 1.00 | 208.87 | 6 |
| ATOM | 7581 | CB | ALA | C | 529 | 31.159 | 130.779 | 28.936 | 1.00 | 108.91 | 6 |
| ATOM | 7582 | C | ALA | C | 529 | 29.386 | 129.768 | 27.498 | 1.00 | 208.87 | 6 |
| ATOM | 7583 | O | ALA | C | 529 | 28.406 | 129.810 | 26.756 | 1.00 | 208.87 | 8 |
| ATOM | 7584 | N | ALA | C | 530 | 30.106 | 128.663 | 27.676 | 1.00 | 84.61 | 7 |
| ATOM | 7585 | CA | ALA | C | 530 | 29.747 | 127.402 | 27.033 | 1.00 | 80.94 | 6 |
| ATOM | 7586 | CB | ALA | C | 530 | 30.852 | 126.936 | 26.058 | 1.00 | 32.73 | 6 |
| ATOM | 7587 | C | ALA | C | 530 | 29.536 | 126.378 | 28.146 | 1.00 | 78.57 | 6 |
| ATOM | 7588 | O | ALA | C | 530 | 29.626 | 125.177 | 27.918 | 1.00 | 78.56 | 8 |
| ATOM | 7589 | N | ALA | C | 531 | 29.252 | 126.877 | 29.351 | 1.00 | 55.15 | 7 |
| ATOM | 7590 | CA | ALA | C | 531 | 29.011 | 126.049 | 30.535 | 1.00 | 52.08 | 6 |
| ATOM | 7591 | CB | ALA | C | 531 | 28.402 | 126.892 | 31.633 | 1.00 | 68.22 | 6 |
| ATOM | 7592 | C | ALA | C | 531 | 28.098 | 124.878 | 30.236 | 1.00 | 50.47 | 6 |
| ATOM | 7593 | O | ALA | C | 531 | 27.437 | 124.863 | 29.208 | 1.00 | 50.11 | 8 |
| ATOM | 7594 | N | MET | C | 532 | 28.050 | 123.911 | 31.147 | 1.00 | 83.34 | 7 |
| ATOM | 7595 | CA | MET | C | 532 | 27.218 | 122.716 | 30.980 | 1.00 | 82.40 | 6 |
| ATOM | 7596 | CB | MET | C | 532 | 25.903 | 123.054 | 30.258 | 1.00 | 107.38 | 6 |
| ATOM | 7597 | CG | MET | C | 532 | 24.800 | 123.637 | 31.124 | 1.00 | 108.62 | 6 |
| ATOM | 7598 | SD | MET | C | 532 | 23.914 | 122.374 | 32.061 | 1.00 | 111.01 | 16 |
| ATOM | 7599 | CE | MET | C | 532 | 22.767 | 121.732 | 30.820 | 1.00 | 108.74 | 6 |
| ATOM | 7600 | C | MET | C | 532 | 27.920 | 121.596 | 30.205 | 1.00 | 80.11 | 6 |
| ATOM | 7601 | O | MET | C | 532 | 28.729 | 121.845 | 29.304 | 1.00 | 79.97 | 8 |
| ATOM | 7602 | N | ASP | C | 533 | 27.597 | 120.358 | 30.561 | 1.00 | 45.22 | 7 |
| ATOM | 7603 | CA | ASP | C | 533 | 28.167 | 119.201 | 29.894 | 1.00 | 42.94 | 6 |
| ATOM | 7604 | CB | ASP | C | 533 | 29.153 | 118.488 | 30.815 | 1.00 | 118.04 | 6 |
| ATOM | 7605 | CG | ASP | C | 533 | 30.377 | 119.314 | 31.092 | 1.00 | 119.82 | 6 |
| ATOM | 7606 | OD1 | ASP | C | 533 | 30.962 | 119.851 | 30.126 | 1.00 | 120.83 | 8 |
| ATOM | 7607 | OD2 | ASP | C | 533 | 30.758 | 119.421 | 32.274 | 1.00 | 120.99 | 8 |
| ATOM | 7608 | C | ASP | C | 533 | 27.080 | 118.227 | 29.470 | 1.00 | 40.69 | 6 |
| ATOM | 7609 | O | ASP | C | 533 | 26.354 | 117.706 | 30.304 | 1.00 | 39.20 | 8 |
| ATOM | 7610 | N | VAL | C | 534 | 26.965 | 117.987 | 28.170 | 1.00 | 45.56 | 7 |
| ATOM | 7611 | CA | VAL | C | 534 | 25.972 | 117.052 | 27.673 | 1.00 | 43.30 | 6 |
| ATOM | 7612 | CB | VAL | C | 534 | 26.208 | 116.733 | 26.180 | 1.00 | 56.00 | 6 |
| ATOM | 7613 | CG1 | VAL | C | 534 | 24.884 | 116.584 | 25.472 | 1.00 | 57.40 | 6 |
| ATOM | 7614 | CG2 | VAL | C | 534 | 27.060 | 117.801 | 25.536 | 1.00 | 55.70 | 6 |
| ATOM | 7615 | C | VAL | C | 534 | 26.176 | 115.768 | 28.479 | 1.00 | 41.63 | 6 |
| ATOM | 7616 | O | VAL | C | 534 | 27.102 | 115.012 | 28.200 | 1.00 | 42.30 | 8 |
| ATOM | 7617 | N | SER | C | 535 | 25.336 | 115.516 | 29.480 | 1.00 | 31.85 | 7 |
| ATOM | 7618 | CA | SER | C | 535 | 25.480 | 114.314 | 30.315 | 1.00 | 30.44 | 6 |
| ATOM | 7619 | CB | SER | C | 535 | 25.198 | 113.049 | 29.505 | 1.00 | 52.78 | 6 |
| ATOM | 7620 | OG | SER | C | 535 | 23.802 | 112.782 | 29.432 | 1.00 | 53.48 | 8 |
| ATOM | 7621 | C | SER | C | 535 | 26.837 | 114.172 | 30.991 | 1.00 | 29.50 | 6 |
| ATOM | 7622 | O | SER | C | 535 | 27.797 | 114.846 | 30.651 | 1.00 | 29.54 | 8 |
| ATOM | 7623 | N | PRO | C | 536 | 26.920 | 113.298 | 31.985 | 1.00 | 57.81 | 7 |
| ATOM | 7624 | CD | PRO | C | 536 | 25.784 | 112.903 | 32.819 | 1.00 | 35.93 | 6 |
| ATOM | 7625 | CA | PRO | C | 536 | 28.163 | 113.074 | 32.709 | 1.00 | 57.22 | 6 |
| ATOM | 7626 | CB | PRO | C | 536 | 27.685 | 112.636 | 34.082 | 1.00 | 35.52 | 6 |
| ATOM | 7627 | CG | PRO | C | 536 | 26.313 | 113.204 | 34.175 | 1.00 | 35.98 | 6 |
| ATOM | 7628 | C | PRO | C | 536 | 29.005 | 112.003 | 32.058 | 1.00 | 57.09 | 6 |
| ATOM | 7629 | O | PRO | C | 536 | 30.212 | 111.962 | 32.257 | 1.00 | 58.55 | 8 |
| ATOM | 7630 | N | LYS | C | 537 | 28.365 | 111.127 | 31.294 | 1.00 | 66.52 | 7 |
| ATOM | 7631 | CA | LYS | C | 537 | 29.072 | 110.045 | 30.620 | 1.00 | 64.77 | 6 |
| ATOM | 7632 | CB | LYS | C | 537 | 28.204 | 109.472 | 29.504 | 1.00 | 67.06 | 6 |
| ATOM | 7633 | CG | LYS | C | 537 | 26.778 | 109.233 | 29.915 | 1.00 | 69.21 | 6 |
| ATOM | 7634 | CD | LYS | C | 537 | 25.935 | 108.782 | 28.744 | 1.00 | 71.51 | 6 |
| ATOM | 7635 | CE | LYS | C | 537 | 24.456 | 108.743 | 29.123 | 1.00 | 73.76 | 6 |
| ATOM | 7636 | NZ | LYS | C | 537 | 24.158 | 107.832 | 30.274 | 1.00 | 76.52 | 7 |
| ATOM | 7637 | C | LYS | C | 537 | 30.382 | 110.539 | 30.018 | 1.00 | 63.46 | 6 |
| ATOM | 7638 | O | LYS | C | 537 | 31.406 | 109.853 | 30.085 | 1.00 | 65.23 | 8 |
| ATOM | 7639 | N | GLN | C | 538 | 30.340 | 111.738 | 29.443 | 1.00 | 14.62 | 7 |
| ATOM | 7640 | CA | GLN | C | 538 | 31.497 | 112.314 | 28.794 | 1.00 | 13.87 | 6 |
| ATOM | 7641 | CB | GLN | C | 538 | 31.140 | 113.679 | 28.237 | 1.00 | 29.72 | 6 |
| ATOM | 7642 | CG | GLN | C | 538 | 30.595 | 114.627 | 29.268 | 1.00 | 28.74 | 6 |
| ATOM | 7643 | CD | GLN | C | 538 | 30.412 | 116.020 | 28.710 | 1.00 | 26.62 | 6 |
| ATOM | 7644 | OE1 | GLN | C | 538 | 31.361 | 116.639 | 28.232 | 1.00 | 26.40 | 8 |
| ATOM | 7645 | NE2 | GLN | C | 538 | 29.189 | 116.522 | 28.766 | 1.00 | 25.10 | 7 |
| ATOM | 7646 | C | GLN | C | 538 | 32.741 | 112.413 | 29.669 | 1.00 | 13.87 | 6 |
| ATOM | 7647 | O | GLN | C | 538 | 33.836 | 112.658 | 29.175 | 1.00 | 14.40 | 8 |
| ATOM | 7648 | N | VAL | C | 539 | 32.598 | 112.238 | 30.969 | 1.00 | 61.78 | 7 |
| ATOM | 7649 | CA | VAL | C | 539 | 33.766 | 112.294 | 31.827 | 1.00 | 62.62 | 6 |
| ATOM | 7650 | CB | VAL | C | 539 | 33.397 | 112.686 | 33.274 | 1.00 | 42.82 | 6 |
| ATOM | 7651 | CG1 | VAL | C | 539 | 34.640 | 112.726 | 34.142 | 1.00 | 43.04 | 6 |
| ATOM | 7652 | CG2 | VAL | C | 539 | 32.720 | 114.012 | 33.289 | 1.00 | 43.33 | 6 |
| ATOM | 7653 | C | VAL | C | 539 | 34.295 | 110.878 | 31.857 | 1.00 | 64.28 | 6 |
| ATOM | 7654 | O | VAL | C | 539 | 35.237 | 110.578 | 32.583 | 1.00 | 67.03 | 8 |
| ATOM | 7655 | N | PHE | C | 540 | 33.697 | 109.997 | 31.064 | 1.00 | 55.62 | 7 |
| ATOM | 7656 | CA | PHE | C | 540 | 34.122 | 108.617 | 31.128 | 1.00 | 55.60 | 6 |
| ATOM | 7657 | CB | PHE | C | 540 | 33.207 | 107.879 | 32.120 | 1.00 | 28.30 | 6 |
| ATOM | 7658 | CG | PHE | C | 540 | 33.315 | 108.391 | 33.538 | 1.00 | 24.65 | 6 |
| ATOM | 7659 | CD1 | PHE | C | 540 | 32.633 | 109.518 | 33.935 | 1.00 | 22.13 | 6 |
| ATOM | 7660 | CD2 | PHE | C | 540 | 34.169 | 107.797 | 34.439 | 1.00 | 25.34 | 6 |
| ATOM | 7661 | CE1 | PHE | C | 540 | 32.809 | 110.044 | 35.188 | 1.00 | 22.70 | 6 |
| ATOM | 7662 | CE2 | PHE | C | 540 | 34.344 | 108.325 | 35.697 | 1.00 | 26.61 | 6 |
| ATOM | 7663 | CZ | PHE | C | 540 | 33.665 | 109.448 | 36.067 | 1.00 | 25.32 | 6 |
| ATOM | 7664 | C | PHE | C | 540 | 34.271 | 107.803 | 29.846 | 1.00 | 56.85 | 6 |
| ATOM | 7665 | O | PHE | C | 540 | 33.837 | 108.214 | 28.764 | 1.00 | 57.37 | 8 |
| ATOM | 7666 | N | SER | C | 541 | 34.913 | 106.641 | 30.009 | 1.00 | 50.86 | 7 |
| ATOM | 7667 | CA | SER | C | 541 | 35.186 | 105.669 | 28.952 | 1.00 | 49.99 | 6 |
| ATOM | 7668 | CB | SER | C | 541 | 36.327 | 104.781 | 29.393 | 1.00 | 61.30 | 6 |
| ATOM | 7669 | OG | SER | C | 541 | 36.199 | 104.522 | 30.780 | 1.00 | 65.14 | 8 |
| ATOM | 7670 | C | SER | C | 541 | 33.971 | 104.800 | 28.664 | 1.00 | 48.71 | 6 |
| ATOM | 7671 | O | SER | C | 541 | 33.198 | 104.473 | 29.563 | 1.00 | 48.33 | 8 |
| ATOM | 7672 | N | LEU | C | 542 | 33.835 | 104.389 | 27.409 | 1.00 | 55.24 | 7 |
| ATOM | 7673 | CA | LEU | C | 542 | 32.697 | 103.590 | 26.976 | 1.00 | 53.98 | 6 |
| ATOM | 7674 | CB | LEU | C | 542 | 32.870 | 103.132 | 25.533 | 1.00 | 17.92 | 6 |
| ATOM | 7675 | CG | LEU | C | 542 | 31.622 | 102.457 | 24.973 | 1.00 | 15.59 | 6 |
| ATOM | 7676 | CD1 | LEU | C | 542 | 30.413 | 103.338 | 25.206 | 1.00 | 15.21 | 6 |
| ATOM | 7677 | CD2 | LEU | C | 542 | 31.804 | 102.196 | 23.501 | 1.00 | 15.84 | 6 |
| ATOM | 7678 | C | LEU | C | 542 | 32.334 | 102.386 | 27.811 | 1.00 | 54.34 | 6 |
| ATOM | 7679 | O | LEU | C | 542 | 31.156 | 102.131 | 28.010 | 1.00 | 55.12 | 8 |
| ATOM | 7680 | N | ASN | C | 543 | 33.309 | 101.622 | 28.291 | 1.00 | 39.01 | 7 |
| ATOM | 7681 | CA | ASN | C | 543 | 32.926 | 100.467 | 29.086 | 1.00 | 39.30 | 6 |
| ATOM | 7682 | CB | ASN | C | 543 | 33.774 | 99.207 | 28.743 | 1.00 | 35.95 | 6 |
| ATOM | 7683 | CG | ASN | C | 543 | 35.259 | 99.387 | 28.936 | 1.00 | 36.82 | 6 |
| ATOM | 7684 | OD1 | ASN | C | 543 | 35.756 | 100.492 | 29.145 | 1.00 | 37.92 | 8 |
| ATOM | 7685 | ND2 | ASN | C | 543 | 35.985 | 98.284 | 28.844 | 1.00 | 35.25 | 7 |
| ATOM | 7686 | C | ASN | C | 543 | 32.826 | 100.718 | 30.583 | 1.00 | 38.44 | 6 |
| ATOM | 7687 | O | ASN | C | 543 | 32.380 | 99.850 | 31.331 | 1.00 | 39.50 | 8 |
| ATOM | 7688 | N | THR | C | 544 | 33.211 | 101.905 | 31.034 | 1.00 | 57.02 | 7 |
| ATOM | 7689 | CA | THR | C | 544 | 33.058 | 102.184 | 32.451 | 1.00 | 56.20 | 6 |
| ATOM | 7690 | CB | THR | C | 544 | 33.889 | 103.384 | 32.944 | 1.00 | 23.10 | 6 |
| ATOM | 7691 | OG1 | THR | C | 544 | 35.242 | 102.966 | 33.159 | 1.00 | 21.43 | 8 |
| ATOM | 7692 | CG2 | THR | C | 544 | 33.332 | 103.924 | 34.261 | 1.00 | 20.88 | 6 |
| ATOM | 7693 | C | THR | C | 544 | 31.601 | 102.529 | 32.580 | 1.00 | 57.34 | 6 |
| ATOM | 7694 | O | THR | C | 544 | 30.917 | 102.020 | 33.457 | 1.00 | 57.84 | 8 |
| ATOM | 7695 | N | ASN | C | 545 | 31.117 | 103.374 | 31.678 | 1.00 | 42.11 | 7 |
| ATOM | 7696 | CA | ASN | C | 545 | 29.720 | 103.776 | 31.713 | 1.00 | 43.29 | 6 |
| ATOM | 7697 | CB | ASN | C | 545 | 29.273 | 104.314 | 30.353 | 1.00 | 52.15 | 6 |
| ATOM | 7698 | CG | ASN | C | 545 | 29.957 | 105.617 | 29.977 | 1.00 | 53.97 | 6 |
| ATOM | 7699 | OD1 | ASN | C | 545 | 29.700 | 106.662 | 30.566 | 1.00 | 54.69 | 8 |
| ATOM | 7700 | ND2 | ASN | C | 545 | 30.835 | 105.556 | 28.988 | 1.00 | 54.19 | 7 |
| ATOM | 7701 | C | ASN | C | 545 | 28.845 | 102.592 | 32.091 | 1.00 | 44.50 | 6 |
| ATOM | 7702 | O | ASN | C | 545 | 27.907 | 102.731 | 32.870 | 1.00 | 46.05 | 8 |
| ATOM | 7703 | N | LEU | C | 546 | 29.179 | 101.420 | 31.561 | 1.00 | 60.59 | 7 |
| ATOM | 7704 | CA | LEU | C | 546 | 28.403 | 100.213 | 31.812 | 1.00 | 60.11 | 6 |
| ATOM | 7705 | CB | LEU | C | 546 | 28.992 | 99.035 | 31.020 | 1.00 | 37.83 | 6 |
| ATOM | 7706 | CG | LEU | C | 546 | 29.088 | 99.200 | 29.493 | 1.00 | 37.99 | 6 |
| ATOM | 7707 | CD1 | LEU | C | 546 | 29.306 | 97.862 | 28.830 | 1.00 | 37.92 | 6 |
| ATOM | 7708 | CD2 | LEU | C | 546 | 27.821 | 99.807 | 28.958 | 1.00 | 37.51 | 6 |
| ATOM | 7709 | C | LEU | C | 546 | 28.232 | 99.818 | 33.274 | 1.00 | 59.65 | 6 |
| ATOM | 7710 | O | LEU | C | 546 | 27.836 | 98.693 | 33.558 | 1.00 | 61.57 | 8 |
| ATOM | 7711 | N | ILE | C | 547 | 28.512 | 100.738 | 34.195 | 1.00 | 28.92 | 7 |
| ATOM | 7712 | CA | ILE | C | 547 | 28.377 | 100.475 | 35.637 | 1.00 | 27.88 | 6 |
| ATOM | 7713 | CB | ILE | C | 547 | 29.623 | 100.935 | 36.422 | 1.00 | 54.96 | 6 |
| ATOM | 7714 | CG2 | ILE | C | 547 | 29.344 | 100.847 | 37.900 | 1.00 | 56.07 | 6 |
| ATOM | 7715 | CG1 | ILE | C | 547 | 30.831 | 100.062 | 36.074 | 1.00 | 54.53 | 6 |
| ATOM | 7716 | CD1 | ILE | C | 547 | 32.127 | 100.537 | 36.711 | 1.00 | 53.36 | 6 |
| ATOM | 7717 | C | ILE | C | 547 | 27.160 | 101.205 | 36.211 | 1.00 | 26.96 | 6 |
| ATOM | 7718 | O | ILE | C | 547 | 27.216 | 102.399 | 36.485 | 1.00 | 25.95 | 8 |
| ATOM | 7719 | N | PRO | C | 548 | 26.065 | 100.473 | 36.451 | 1.00 | 55.58 | 7 |
| ATOM | 7720 | CD | PRO | C | 548 | 26.037 | 99.021 | 36.671 | 1.00 | 35.60 | 6 |
| ATOM | 7721 | CA | PRO | C | 548 | 24.846 | 101.063 | 36.977 | 1.00 | 55.04 | 6 |
| ATOM | 7722 | CB | PRO | C | 548 | 23.954 | 99.849 | 37.190 | 1.00 | 33.94 | 6 |
| ATOM | 7723 | CG | PRO | C | 548 | 24.900 | 98.866 | 37.658 | 1.00 | 34.54 | 6 |
| ATOM | 7724 | C | PRO | C | 548 | 25.049 | 101.897 | 38.222 | 1.00 | 55.38 | 6 |
| ATOM | 7725 | O | PRO | C | 548 | 24.279 | 102.819 | 38.473 | 1.00 | 57.14 | 8 |
| ATOM | 7726 | N | PHE | C | 549 | 26.061 | 101.611 | 39.022 | 1.00 | 32.51 | 7 |
| ATOM | 7727 | CA | PHE | C | 549 | 26.220 | 102.467 | 40.191 | 1.00 | 33.20 | 6 |
| ATOM | 7728 | CB | PHE | C | 549 | 25.645 | 101.798 | 41.446 | 1.00 | 27.42 | 6 |
| ATOM | 7729 | CG | PHE | C | 549 | 24.130 | 101.793 | 41.503 | 1.00 | 24.30 | 6 |
| ATOM | 7730 | CD1 | PHE | C | 549 | 23.387 | 100.840 | 40.798 | 1.00 | 23.85 | 6 |
| ATOM | 7731 | CD2 | PHE | C | 549 | 23.449 | 102.708 | 42.295 | 1.00 | 22.95 | 6 |
| ATOM | 7732 | CE1 | PHE | C | 549 | 21.989 | 100.795 | 40.889 | 1.00 | 22.47 | 6 |
| ATOM | 7733 | CE2 | PHE | C | 549 | 22.058 | 102.665 | 42.387 | 1.00 | 23.27 | 6 |
| ATOM | 7734 | CZ | PHE | C | 549 | 21.331 | 101.704 | 41.682 | 1.00 | 22.31 | 6 |
| ATOM | 7735 | C | PHE | C | 549 | 27.650 | 102.941 | 40.445 | 1.00 | 33.28 | 6 |
| ATOM | 7736 | O | PHE | C | 549 | 28.181 | 102.811 | 41.549 | 1.00 | 33.61 | 8 |
| ATOM | 7737 | N | LEU | C | 550 | 28.252 | 103.525 | 39.412 | 1.00 | 29.32 | 7 |
| ATOM | 7738 | CA | LEU | C | 550 | 29.619 | 104.025 | 39.485 | 1.00 | 29.00 | 6 |
| ATOM | 7739 | CB | LEU | C | 550 | 30.009 | 104.696 | 38.156 | 1.00 | 46.50 | 6 |
| ATOM | 7740 | CG | LEU | C | 550 | 31.458 | 105.140 | 37.930 | 1.00 | 45.08 | 6 |
| ATOM | 7741 | CD1 | LEU | C | 550 | 32.372 | 103.940 | 37.982 | 1.00 | 46.72 | 6 |
| ATOM | 7742 | CD2 | LEU | C | 550 | 31.594 | 105.833 | 36.586 | 1.00 | 43.39 | 6 |
| ATOM | 7743 | C | LEU | C | 550 | 29.723 | 105.018 | 40.621 | 1.00 | 29.29 | 6 |
| ATOM | 7744 | O | LEU | C | 550 | 30.572 | 104.864 | 41.492 | 1.00 | 27.19 | 8 |
| ATOM | 7745 | N | GLU | C | 551 | 28.842 | 106.019 | 40.613 | 1.00 | 28.00 | 7 |
| ATOM | 7746 | CA | GLU | C | 551 | 28.824 | 107.078 | 41.627 | 1.00 | 30.78 | 6 |
| ATOM | 7747 | CB | GLU | C | 551 | 27.403 | 107.611 | 41.844 | 1.00 | 103.26 | 6 |
| ATOM | 7748 | CG | GLU | C | 551 | 26.502 | 107.570 | 40.629 | 1.00 | 106.00 | 6 |
| ATOM | 7749 | CD | GLU | C | 551 | 25.687 | 106.302 | 40.575 | 1.00 | 108.97 | 6 |
| ATOM | 7750 | OE1 | GLU | C | 551 | 26.287 | 105.213 | 40.571 | 1.00 | 108.75 | 8 |
| ATOM | 7751 | OE2 | GLU | C | 551 | 24.443 | 106.392 | 40.544 | 1.00 | 110.84 | 8 |
| ATOM | 7752 | C | GLU | C | 551 | 29.395 | 106.666 | 42.978 | 1.00 | 31.42 | 6 |
| ATOM | 7753 | O | GLU | C | 551 | 30.171 | 107.403 | 43.564 | 1.00 | 31.29 | 8 |
| ATOM | 7754 | N | HIS | C | 552 | 29.010 | 105.491 | 43.466 | 1.00 | 50.43 | 7 |
| ATOM | 7755 | CA | HIS | C | 552 | 29.490 | 105.001 | 44.754 | 1.00 | 51.46 | 6 |
| ATOM | 7756 | CB | HIS | C | 552 | 28.470 | 104.047 | 45.341 | 1.00 | 54.12 | 6 |
| ATOM | 7757 | CG | HIS | C | 552 | 27.067 | 104.544 | 45.220 | 1.00 | 54.38 | 6 |
| ATOM | 7758 | CD2 | HIS | C | 552 | 26.123 | 104.312 | 44.276 | 1.00 | 55.28 | 6 |
| ATOM | 7759 | ND1 | HIS | C | 552 | 26.505 | 105.428 | 46.115 | 1.00 | 54.20 | 7 |
| ATOM | 7760 | CE1 | HIS | C | 552 | 25.274 | 105.715 | 45.728 | 1.00 | 55.46 | 6 |
| ATOM | 7761 | NE2 | HIS | C | 552 | 25.019 | 105.054 | 44.614 | 1.00 | 55.94 | 7 |
| ATOM | 7762 | C | HIS | C | 552 | 30.830 | 104.306 | 44.599 | 1.00 | 51.10 | 6 |
| ATOM | 7763 | O | HIS | C | 552 | 31.788 | 104.632 | 45.288 | 1.00 | 53.17 | 8 |
| ATOM | 7764 | N | ASP | C | 553 | 30.893 | 103.337 | 43.701 | 1.00 | 52.36 | 7 |
| ATOM | 7765 | CA | ASP | C | 553 | 32.134 | 102.629 | 43.435 | 1.00 | 51.21 | 6 |
| ATOM | 7766 | CB | ASP | C | 553 | 32.087 | 102.026 | 42.036 | 1.00 | 32.32 | 6 |
| ATOM | 7767 | CG | ASP | C | 553 | 31.310 | 100.750 | 41.982 | 1.00 | 32.47 | 6 |
| ATOM | 7768 | OD1 | ASP | C | 553 | 30.513 | 100.478 | 42.896 | 1.00 | 33.28 | 8 |
| ATOM | 7769 | OD2 | ASP | C | 553 | 31.498 | 100.017 | 41.006 | 1.00 | 31.58 | 8 |
| ATOM | 7770 | C | ASP | C | 553 | 33.331 | 103.576 | 43.504 | 1.00 | 50.90 | 6 |
| ATOM | 7771 | O | ASP | C | 553 | 33.383 | 104.580 | 42.786 | 1.00 | 51.83 | 8 |
| ATOM | 7772 | N | ASP | C | 554 | 34.290 | 103.251 | 44.367 | 1.00 | 33.65 | 7 |
| ATOM | 7773 | CA | ASP | C | 554 | 35.501 | 104.050 | 44.509 | 1.00 | 31.93 | 6 |
| ATOM | 7774 | CB | ASP | C | 554 | 36.383 | 103.475 | 45.612 | 1.00 | 68.27 | 6 |
| ATOM | 7775 | CG | ASP | C | 554 | 37.684 | 104.218 | 45.756 | 1.00 | 72.84 | 6 |
| ATOM | 7776 | OD1 | ASP | C | 554 | 38.411 | 104.320 | 44.749 | 1.00 | 73.81 | 8 |
| ATOM | 7777 | OD2 | ASP | C | 554 | 37.984 | 104.697 | 46.872 | 1.00 | 76.46 | 8 |
| ATOM | 7778 | C | ASP | C | 554 | 36.224 | 103.971 | 43.181 | 1.00 | 30.26 | 6 |
| ATOM | 7779 | O | ASP | C | 554 | 36.417 | 102.881 | 42.639 | 1.00 | 29.04 | 8 |
| ATOM | 7780 | N | ALA | C | 555 | 36.608 | 105.130 | 42.653 | 1.00 | 44.51 | 7 |
| ATOM | 7781 | CA | ALA | C | 555 | 37.301 | 105.204 | 41.370 | 1.00 | 42.66 | 6 |
| ATOM | 7782 | CB | ALA | C | 555 | 38.172 | 106.459 | 41.315 | 1.00 | 29.64 | 6 |
| ATOM | 7783 | C | ALA | C | 555 | 38.146 | 103.956 | 41.128 | 1.00 | 41.50 | 6 |
| ATOM | 7784 | O | ALA | C | 555 | 37.787 | 103.107 | 40.318 | 1.00 | 41.76 | 8 |
| ATOM | 7785 | N | ASN | C | 556 | 39.254 | 103.842 | 41.853 | 1.00 | 29.75 | 7 |
| ATOM | 7786 | CA | ASN | C | 556 | 40.163 | 102.705 | 41.735 | 1.00 | 29.63 | 6 |
| ATOM | 7787 | CB | ASN | C | 556 | 41.039 | 102.614 | 42.984 | 1.00 | 56.62 | 6 |
| ATOM | 7788 | CG | ASN | C | 556 | 42.286 | 101.789 | 42.758 | 1.00 | 57.90 | 6 |
| ATOM | 7789 | OD1 | ASN | C | 556 | 42.215 | 100.622 | 42.368 | 1.00 | 58.43 | 8 |
| ATOM | 7790 | ND2 | ASN | C | 556 | 43.443 | 102.395 | 43.000 | 1.00 | 57.43 | 7 |
| ATOM | 7791 | C | ASN | C | 556 | 39.482 | 101.351 | 41.500 | 1.00 | 27.87 | 6 |
| ATOM | 7792 | O | ASN | C | 556 | 39.574 | 100.778 | 40.417 | 1.00 | 26.94 | 8 |
| ATOM | 7793 | N | ARG | C | 557 | 38.807 | 100.831 | 42.515 | 1.00 | 65.96 | 7 |
| ATOM | 7794 | CA | ARG | C | 557 | 38.147 | 99.544 | 42.367 | 1.00 | 64.43 | 6 |
| ATOM | 7795 | CB | ARG | C | 557 | 37.297 | 99.222 | 43.582 | 1.00 | 26.99 | 6 |
| ATOM | 7796 | CG | ARG | C | 557 | 37.075 | 97.758 | 43.729 | 1.00 | 24.27 | 6 |
| ATOM | 7797 | CD | ARG | C | 557 | 38.387 | 97.118 | 44.108 | 1.00 | 22.59 | 6 |
| ATOM | 7798 | NE | ARG | C | 557 | 38.365 | 95.665 | 44.016 | 1.00 | 22.25 | 7 |
| ATOM | 7799 | CZ | ARG | C | 557 | 39.348 | 94.890 | 44.455 | 1.00 | 22.39 | 6 |
| ATOM | 7800 | NH1 | ARG | C | 557 | 40.414 | 95.442 | 45.019 | 1.00 | 22.25 | 7 |
| ATOM | 7801 | NH2 | ARG | C | 557 | 39.283 | 93.573 | 44.309 | 1.00 | 22.91 | 7 |
| ATOM | 7802 | C | ARG | C | 557 | 37.253 | 99.524 | 41.144 | 1.00 | 64.64 | 6 |
| ATOM | 7803 | O | ARG | C | 557 | 37.111 | 98.495 | 40.491 | 1.00 | 66.31 | 8 |
| ATOM | 7804 | N | ALA | C | 558 | 36.634 | 100.661 | 40.847 | 1.00 | 19.66 | 7 |
| ATOM | 7805 | CA | ALA | C | 558 | 35.756 | 100.756 | 39.690 | 1.00 | 18.12 | 6 |
| ATOM | 7806 | CB | ALA | C | 558 | 35.153 | 102.159 | 39.615 | 1.00 | 46.30 | 6 |
| ATOM | 7807 | C | ALA | C | 558 | 36.490 | 100.413 | 38.380 | 1.00 | 17.77 | 6 |
| ATOM | 7808 | O | ALA | C | 558 | 35.847 | 100.003 | 37.411 | 1.00 | 15.92 | 8 |
| ATOM | 7809 | N | LEU | C | 559 | 37.824 | 100.579 | 38.358 | 1.00 | 13.87 | 7 |
| ATOM | 7810 | CA | LEU | C | 559 | 38.633 | 100.284 | 37.167 | 1.00 | 13.87 | 6 |
| ATOM | 7811 | CB | LEU | C | 559 | 40.013 | 100.986 | 37.209 | 1.00 | 13.87 | 6 |
| ATOM | 7812 | CG | LEU | C | 559 | 41.269 | 100.503 | 37.978 | 1.00 | 14.77 | 6 |
| ATOM | 7813 | CD1 | LEU | C | 559 | 41.522 | 99.032 | 37.759 | 1.00 | 14.05 | 6 |
| ATOM | 7814 | CD2 | LEU | C | 559 | 42.504 | 101.289 | 37.513 | 1.00 | 13.87 | 6 |
| ATOM | 7815 | C | LEU | C | 559 | 38.822 | 98.781 | 37.046 | 1.00 | 13.87 | 6 |
| ATOM | 7816 | O | LEU | C | 559 | 39.047 | 98.256 | 35.960 | 1.00 | 15.26 | 8 |
| ATOM | 7817 | N | MET | C | 560 | 38.754 | 98.082 | 38.171 | 1.00 | 13.87 | 7 |
| ATOM | 7818 | CA | MET | C | 560 | 38.890 | 96.637 | 38.140 | 1.00 | 13.87 | 6 |
| ATOM | 7819 | CB | MET | C | 560 | 39.149 | 96.092 | 39.532 | 1.00 | 24.02 | 6 |
| ATOM | 7820 | CG | MET | C | 560 | 40.602 | 95.859 | 39.795 | 1.00 | 24.05 | 6 |
| ATOM | 7821 | SD | MET | C | 560 | 40.804 | 95.282 | 41.440 | 1.00 | 24.44 | 16 |
| ATOM | 7822 | CE | MET | C | 560 | 41.402 | 96.799 | 42.230 | 1.00 | 24.05 | 6 |
| ATOM | 7823 | C | MET | C | 560 | 37.625 | 96.021 | 37.576 | 1.00 | 13.87 | 6 |
| ATOM | 7824 | O | MET | C | 560 | 37.547 | 94.817 | 37.365 | 1.00 | 13.87 | 8 |
| ATOM | 7825 | N | GLY | C | 561 | 36.627 | 96.862 | 37.350 | 1.00 | 28.68 | 7 |
| ATOM | 7826 | CA | GLY | C | 561 | 35.384 | 96.393 | 36.781 | 1.00 | 29.22 | 6 |
| ATOM | 7827 | C | GLY | C | 561 | 35.362 | 96.946 | 35.375 | 1.00 | 30.05 | 6 |
| ATOM | 7828 | O | GLY | C | 561 | 35.040 | 96.258 | 34.413 | 1.00 | 30.74 | 8 |
| ATOM | 7829 | N | SER | C | 562 | 35.710 | 98.216 | 35.258 | 1.00 | 21.52 | 7 |
| ATOM | 7830 | CA | SER | C | 562 | 35.746 | 98.842 | 33.965 | 1.00 | 23.76 | 6 |
| ATOM | 7831 | CB | SER | C | 562 | 36.439 | 100.197 | 34.076 | 1.00 | 50.05 | 6 |
| ATOM | 7832 | OG | SER | C | 562 | 36.642 | 100.776 | 32.800 | 1.00 | 54.47 | 8 |
| ATOM | 7833 | C | SER | C | 562 | 36.522 | 97.901 | 33.041 | 1.00 | 23.13 | 6 |
| ATOM | 7834 | O | SER | C | 562 | 36.001 | 97.465 | 32.014 | 1.00 | 23.33 | 8 |
| ATOM | 7835 | N | ASN | C | 563 | 37.753 | 97.563 | 33.423 | 1.00 | 14.06 | 7 |
| ATOM | 7836 | CA | ASN | C | 563 | 38.593 | 96.674 | 32.617 | 1.00 | 14.75 | 6 |
| ATOM | 7837 | CB | ASN | C | 563 | 40.015 | 96.557 | 33.186 | 1.00 | 59.25 | 6 |
| ATOM | 7838 | CG | ASN | C | 563 | 40.754 | 97.873 | 33.222 | 1.00 | 63.28 | 6 |
| ATOM | 7839 | OD1 | ASN | C | 563 | 40.354 | 98.848 | 32.588 | 1.00 | 67.18 | 8 |
| ATOM | 7840 | ND2 | ASN | C | 563 | 41.857 | 97.902 | 33.959 | 1.00 | 63.33 | 7 |
| ATOM | 7841 | C | ASN | C | 563 | 38.006 | 95.285 | 32.598 | 1.00 | 13.87 | 6 |
| ATOM | 7842 | O | ASN | C | 563 | 37.452 | 94.842 | 31.617 | 1.00 | 13.87 | 8 |
| ATOM | 7843 | N | MET | C | 564 | 38.161 | 94.606 | 33.716 | 1.00 | 17.31 | 7 |
| ATOM | 7844 | CA | MET | C | 564 | 37.688 | 93.262 | 33.895 | 1.00 | 16.52 | 6 |
| ATOM | 7845 | CB | MET | C | 564 | 37.319 | 93.087 | 35.333 | 1.00 | 26.95 | 6 |
| ATOM | 7846 | CG | MET | C | 564 | 36.910 | 91.729 | 35.687 | 1.00 | 28.06 | 6 |
| ATOM | 7847 | SD | MET | C | 564 | 36.957 | 91.733 | 37.424 | 1.00 | 31.38 | 16 |
| ATOM | 7848 | CE | MET | C | 564 | 38.697 | 91.983 | 37.652 | 1.00 | 32.35 | 6 |
| ATOM | 7849 | C | MET | C | 564 | 36.507 | 92.920 | 33.032 | 1.00 | 16.05 | 6 |
| ATOM | 7850 | O | MET | C | 564 | 36.483 | 91.892 | 32.381 | 1.00 | 16.44 | 8 |
| ATOM | 7851 | N | GLN | C | 565 | 35.509 | 93.778 | 33.045 | 1.00 | 26.07 | 7 |
| ATOM | 7852 | CA | GLN | C | 565 | 34.311 | 93.569 | 32.253 | 1.00 | 26.11 | 6 |
| ATOM | 7853 | CB | GLN | C | 565 | 33.591 | 94.916 | 32.178 | 1.00 | 18.22 | 6 |
| ATOM | 7854 | CG | GLN | C | 565 | 32.342 | 94.971 | 31.365 | 1.00 | 19.82 | 6 |
| ATOM | 7855 | CD | GLN | C | 565 | 31.359 | 95.923 | 31.975 | 1.00 | 20.65 | 6 |
| ATOM | 7856 | OE1 | GLN | C | 565 | 31.736 | 96.987 | 32.462 | 1.00 | 19.74 | 8 |
| ATOM | 7857 | NE2 | GLN | C | 565 | 30.086 | 95.551 | 31.956 | 1.00 | 20.72 | 7 |
| ATOM | 7858 | C | GLN | C | 565 | 34.670 | 93.011 | 30.859 | 1.00 | 25.51 | 6 |
| ATOM | 7859 | O | GLN | C | 565 | 34.077 | 92.049 | 30.383 | 1.00 | 24.62 | 8 |
| ATOM | 7860 | N | THR | C | 566 | 35.677 | 93.612 | 30.235 | 1.00 | 24.10 | 7 |
| ATOM | 7861 | CA | THR | C | 566 | 36.160 | 93.219 | 28.909 | 1.00 | 23.98 | 6 |
| ATOM | 7862 | CB | THR | C | 566 | 37.506 | 93.906 | 28.548 | 1.00 | 20.36 | 6 |
| ATOM | 7863 | OG1 | THR | C | 566 | 37.422 | 95.326 | 28.720 | 1.00 | 20.36 | 8 |
| ATOM | 7864 | CG2 | THR | C | 566 | 37.856 | 93.608 | 27.122 | 1.00 | 20.36 | 6 |
| ATOM | 7865 | C | THR | C | 566 | 36.435 | 91.731 | 28.805 | 1.00 | 24.17 | 6 |
| ATOM | 7866 | O | THR | C | 566 | 36.010 | 91.071 | 27.870 | 1.00 | 24.04 | 8 |
| ATOM | 7867 | N | GLN | C | 567 | 37.187 | 91.226 | 29.769 | 1.00 | 13.87 | 7 |
| ATOM | 7868 | CA | GLN | C | 567 | 37.573 | 89.834 | 29.809 | 1.00 | 13.87 | 6 |
| ATOM | 7869 | CB | GLN | C | 567 | 38.576 | 89.630 | 30.941 | 1.00 | 44.07 | 6 |
| ATOM | 7870 | CG | GLN | C | 567 | 39.702 | 90.652 | 30.918 | 1.00 | 47.21 | 6 |
| ATOM | 7871 | CD | GLN | C | 567 | 40.555 | 90.643 | 32.173 | 1.00 | 49.95 | 6 |
| ATOM | 7872 | OE1 | GLN | C | 567 | 40.059 | 90.818 | 33.285 | 1.00 | 50.29 | 8 |
| ATOM | 7873 | NE2 | GLN | C | 567 | 41.852 | 90.451 | 31.996 | 1.00 | 51.77 | 7 |
| ATOM | 7874 | C | GLN | C | 567 | 36.394 | 88.894 | 29.967 | 1.00 | 13.87 | 6 |
| ATOM | 7875 | O | GLN | C | 567 | 36.579 | 87.756 | 30.369 | 1.00 | 13.87 | 8 |
| ATOM | 7876 | N | ALA | C | 568 | 35.194 | 89.381 | 29.644 | 1.00 | 28.36 | 7 |
| ATOM | 7877 | CA | ALA | C | 568 | 33.937 | 88.620 | 29.728 | 1.00 | 28.85 | 6 |
| ATOM | 7878 | CB | ALA | C | 568 | 32.827 | 89.392 | 29.043 | 1.00 | 75.57 | 6 |
| ATOM | 7879 | C | ALA | C | 568 | 34.004 | 87.207 | 29.148 | 1.00 | 31.00 | 6 |
| ATOM | 7880 | O | ALA | C | 568 | 34.664 | 86.351 | 29.715 | 1.00 | 32.02 | 8 |
| ATOM | 7881 | N | VAL | C | 569 | 33.305 | 86.974 | 28.033 | 1.00 | 22.44 | 7 |
| ATOM | 7882 | CA | VAL | C | 569 | 33.259 | 85.672 | 27.332 | 1.00 | 24.38 | 6 |
| ATOM | 7883 | CB | VAL | C | 569 | 34.054 | 84.547 | 28.049 | 1.00 | 38.29 | 6 |
| ATOM | 7884 | CG1 | VAL | C | 569 | 33.295 | 84.039 | 29.253 | 1.00 | 39.66 | 6 |
| ATOM | 7885 | CG2 | VAL | C | 569 | 34.321 | 83.413 | 27.085 | 1.00 | 39.89 | 6 |
| ATOM | 7886 | C | VAL | C | 569 | 31.831 | 85.164 | 27.129 | 1.00 | 25.75 | 6 |
| ATOM | 7887 | O | VAL | C | 569 | 31.252 | 84.512 | 27.996 | 1.00 | 25.33 | 8 |
| ATOM | 7888 | N | PRO | C | 570 | 31.265 | 85.427 | 25.948 | 1.00 | 29.95 | 7 |
| ATOM | 7889 | CD | PRO | C | 570 | 32.055 | 85.698 | 24.740 | 1.00 | 50.59 | 6 |
| ATOM | 7890 | CA | PRO | C | 570 | 29.908 | 85.027 | 25.579 | 1.00 | 31.13 | 6 |
| ATOM | 7891 | CB | PRO | C | 570 | 29.953 | 85.013 | 24.055 | 1.00 | 50.91 | 6 |
| ATOM | 7892 | CG | PRO | C | 570 | 31.394 | 84.798 | 23.750 | 1.00 | 50.20 | 6 |
| ATOM | 7893 | C | PRO | C | 570 | 29.480 | 83.694 | 26.138 | 1.00 | 31.83 | 6 |
| ATOM | 7894 | O | PRO | C | 570 | 30.207 | 82.707 | 26.024 | 1.00 | 31.32 | 8 |
| ATOM | 7895 | N | LEU | C | 571 | 28.292 | 83.686 | 26.736 | 1.00 | 31.25 | 7 |
| ATOM | 7896 | CA | LEU | C | 571 | 27.708 | 82.487 | 27.320 | 1.00 | 32.79 | 6 |
| ATOM | 7897 | CB | LEU | C | 571 | 27.247 | 82.729 | 28.764 | 1.00 | 13.87 | 6 |
| ATOM | 7898 | CG | LEU | C | 571 | 28.097 | 83.181 | 29.952 | 1.00 | 13.87 | 6 |
| ATOM | 7899 | CD1 | LEU | C | 571 | 28.730 | 84.518 | 29.670 | 1.00 | 13.87 | 6 |
| ATOM | 7900 | CD2 | LEU | C | 571 | 27.205 | 83.294 | 31.181 | 1.00 | 13.87 | 6 |
| ATOM | 7901 | C | LEU | C | 571 | 26.469 | 82.104 | 26.510 | 1.00 | 36.27 | 6 |
| ATOM | 7902 | O | LEU | C | 571 | 26.038 | 82.839 | 25.613 | 1.00 | 35.73 | 8 |
| ATOM | 7903 | N | ILE | C | 572 | 25.907 | 80.947 | 26.851 | 1.00 | 37.31 | 7 |
| ATOM | 7904 | CA | ILE | C | 572 | 24.691 | 80.430 | 26.233 | 1.00 | 40.16 | 6 |
| ATOM | 7905 | CB | ILE | C | 572 | 24.666 | 78.898 | 26.248 | 1.00 | 48.31 | 6 |
| ATOM | 7906 | CG2 | ILE | C | 572 | 23.847 | 78.371 | 25.105 | 1.00 | 49.64 | 6 |
| ATOM | 7907 | CG1 | ILE | C | 572 | 26.078 | 78.357 | 26.099 | 1.00 | 50.85 | 6 |
| ATOM | 7908 | CD1 | ILE | C | 572 | 26.167 | 76.917 | 26.439 | 1.00 | 53.55 | 6 |
| ATOM | 7909 | C | ILE | C | 572 | 23.626 | 80.906 | 27.206 | 1.00 | 42.12 | 6 |
| ATOM | 7910 | O | ILE | C | 572 | 23.315 | 80.207 | 28.175 | 1.00 | 44.93 | 8 |
| ATOM | 7911 | N | ARG | C | 573 | 23.090 | 82.097 | 26.952 | 1.00 | 47.21 | 7 |
| ATOM | 7912 | CA | ARG | C | 573 | 22.064 | 82.732 | 27.791 | 1.00 | 48.00 | 6 |
| ATOM | 7913 | CB | ARG | C | 573 | 21.563 | 81.804 | 28.904 | 1.00 | 177.52 | 6 |
| ATOM | 7914 | CG | ARG | C | 573 | 20.346 | 80.973 | 28.541 | 1.00 | 182.92 | 6 |
| ATOM | 7915 | CD | ARG | C | 573 | 20.107 | 79.866 | 29.558 | 1.00 | 188.88 | 6 |
| ATOM | 7916 | NE | ARG | C | 573 | 18.780 | 79.269 | 29.433 | 1.00 | 196.42 | 7 |
| ATOM | 7917 | CZ | ARG | C | 573 | 18.253 | 78.823 | 28.295 | 1.00 | 201.00 | 6 |
| ATOM | 7918 | NH1 | ARG | C | 573 | 18.936 | 78.901 | 27.160 | 1.00 | 202.35 | 7 |
| ATOM | 7919 | NH2 | ARG | C | 573 | 17.036 | 78.293 | 28.292 | 1.00 | 203.74 | 7 |
| ATOM | 7920 | C | ARG | C | 573 | 22.711 | 83.940 | 28.410 | 1.00 | 45.92 | 6 |
| ATOM | 7921 | O | ARG | C | 573 | 22.941 | 83.986 | 29.612 | 1.00 | 44.53 | 8 |
| ATOM | 7922 | N | ALA | C | 574 | 22.990 | 84.926 | 27.575 | 1.00 | 36.56 | 7 |
| ATOM | 7923 | CA | ALA | C | 574 | 23.655 | 86.123 | 28.026 | 1.00 | 36.57 | 6 |
| ATOM | 7924 | CB | ALA | C | 574 | 24.060 | 86.932 | 26.848 | 1.00 | 28.73 | 6 |
| ATOM | 7925 | C | ALA | C | 574 | 22.884 | 86.994 | 29.000 | 1.00 | 36.13 | 6 |
| ATOM | 7926 | O | ALA | C | 574 | 23.401 | 87.345 | 30.057 | 1.00 | 37.99 | 8 |
| ATOM | 7927 | N | GLN | C | 575 | 21.652 | 87.350 | 28.663 | 1.00 | 22.89 | 7 |
| ATOM | 7928 | CA | GLN | C | 575 | 20.853 | 88.218 | 29.535 | 1.00 | 20.60 | 6 |
| ATOM | 7929 | CB | GLN | C | 575 | 20.633 | 87.599 | 30.921 | 1.00 | 41.70 | 6 |
| ATOM | 7930 | CG | GLN | C | 575 | 20.953 | 86.125 | 31.064 | 1.00 | 41.79 | 6 |
| ATOM | 7931 | CD | GLN | C | 575 | 20.832 | 85.685 | 32.491 | 1.00 | 41.32 | 6 |
| ATOM | 7932 | OE1 | GLN | C | 575 | 21.412 | 86.300 | 33.374 | 1.00 | 42.31 | 8 |
| ATOM | 7933 | NE2 | GLN | C | 575 | 20.074 | 84.627 | 32.732 | 1.00 | 40.07 | 7 |
| ATOM | 7934 | C | GLN | C | 575 | 21.542 | 89.557 | 29.756 | 1.00 | 19.44 | 6 |
| ATOM | 7935 | O | GLN | C | 575 | 22.702 | 89.609 | 30.144 | 1.00 | 19.35 | 8 |
| ATOM | 7936 | N | ALA | C | 576 | 20.836 | 90.648 | 29.514 | 1.00 | 19.52 | 7 |
| ATOM | 7937 | CA | ALA | C | 576 | 21.432 | 91.947 | 29.749 | 1.00 | 19.48 | 6 |
| ATOM | 7938 | CB | ALA | C | 576 | 20.508 | 93.042 | 29.267 | 1.00 | 59.31 | 6 |
| ATOM | 7939 | C | ALA | C | 576 | 21.554 | 92.006 | 31.252 | 1.00 | 19.91 | 6 |
| ATOM | 7940 | O | ALA | C | 576 | 21.283 | 91.029 | 31.937 | 1.00 | 19.51 | 8 |
| ATOM | 7941 | N | PRO | C | 577 | 22.007 | 93.128 | 31.791 | 1.00 | 59.79 | 7 |
| ATOM | 7942 | CD | PRO | C | 577 | 22.962 | 94.057 | 31.168 | 1.00 | 54.76 | 6 |
| ATOM | 7943 | CA | PRO | C | 577 | 22.100 | 93.167 | 33.255 | 1.00 | 61.36 | 6 |
| ATOM | 7944 | CB | PRO | C | 577 | 23.353 | 93.996 | 33.493 | 1.00 | 55.62 | 6 |
| ATOM | 7945 | CG | PRO | C | 577 | 23.350 | 94.932 | 32.323 | 1.00 | 56.36 | 6 |
| ATOM | 7946 | C | PRO | C | 577 | 20.824 | 93.813 | 33.827 | 1.00 | 62.40 | 6 |
| ATOM | 7947 | O | PRO | C | 577 | 20.307 | 94.765 | 33.242 | 1.00 | 62.98 | 8 |
| ATOM | 7948 | N | VAL | C | 578 | 20.315 | 93.302 | 34.953 | 1.00 | 42.71 | 7 |
| ATOM | 7949 | CA | VAL | C | 578 | 19.086 | 93.839 | 35.547 | 1.00 | 41.26 | 6 |
| ATOM | 7950 | CB | VAL | C | 578 | 18.812 | 93.265 | 36.946 | 1.00 | 30.71 | 6 |
| ATOM | 7951 | CG1 | VAL | C | 578 | 17.396 | 93.623 | 37.368 | 1.00 | 30.48 | 6 |
| ATOM | 7952 | CG2 | VAL | C | 578 | 18.969 | 91.759 | 36.936 | 1.00 | 29.50 | 6 |
| ATOM | 7953 | C | VAL | C | 578 | 19.117 | 95.351 | 35.653 | 1.00 | 40.82 | 6 |
| ATOM | 7954 | O | VAL | C | 578 | 18.285 | 96.033 | 35.074 | 1.00 | 41.24 | 8 |
| ATOM | 7955 | N | VAL | C | 579 | 20.068 | 95.872 | 36.408 | 1.00 | 29.07 | 7 |
| ATOM | 7956 | CA | VAL | C | 579 | 20.219 | 97.314 | 36.557 | 1.00 | 28.65 | 6 |
| ATOM | 7957 | CB | VAL | C | 579 | 20.898 | 97.592 | 37.860 | 1.00 | 24.77 | 6 |
| ATOM | 7958 | CG1 | VAL | C | 579 | 20.596 | 98.993 | 38.326 | 1.00 | 24.40 | 6 |
| ATOM | 7959 | CG2 | VAL | C | 579 | 20.453 | 96.540 | 38.867 | 1.00 | 26.25 | 6 |
| ATOM | 7960 | C | VAL | C | 579 | 21.170 | 97.595 | 35.420 | 1.00 | 28.84 | 6 |
| ATOM | 7961 | O | VAL | C | 579 | 22.019 | 96.754 | 35.178 | 1.00 | 29.03 | 8 |
| ATOM | 7962 | N | MET | C | 580 | 21.098 | 98.724 | 34.716 | 1.00 | 36.94 | 7 |
| ATOM | 7963 | CA | MET | C | 580 | 22.044 | 98.816 | 33.609 | 1.00 | 37.38 | 6 |
| ATOM | 7964 | CB | MET | C | 580 | 21.596 | 97.821 | 32.538 | 1.00 | 40.20 | 6 |
| ATOM | 7965 | CG | MET | C | 580 | 20.188 | 97.982 | 32.025 | 1.00 | 40.57 | 6 |
| ATOM | 7966 | SD | MET | C | 580 | 19.791 | 96.643 | 30.875 | 1.00 | 42.09 | 16 |
| ATOM | 7967 | CE | MET | C | 580 | 19.292 | 97.526 | 29.441 | 1.00 | 42.42 | 6 |
| ATOM | 7968 | C | MET | C | 580 | 22.586 | 100.046 | 32.870 | 1.00 | 38.01 | 6 |
| ATOM | 7969 | O | MET | C | 580 | 23.401 | 99.856 | 31.971 | 1.00 | 38.73 | 8 |
| ATOM | 7970 | N | THR | C | 581 | 22.205 | 101.277 | 33.195 | 1.00 | 41.27 | 7 |
| ATOM | 7971 | CA | THR | C | 581 | 22.759 | 102.406 | 32.428 | 1.00 | 40.63 | 6 |
| ATOM | 7972 | CB | THR | C | 581 | 24.294 | 102.411 | 32.477 | 1.00 | 23.18 | 6 |
| ATOM | 7973 | OG1 | THR | C | 581 | 24.735 | 102.856 | 33.762 | 1.00 | 22.74 | 8 |
| ATOM | 7974 | CG2 | THR | C | 581 | 24.854 | 103.303 | 31.386 | 1.00 | 23.31 | 6 |
| ATOM | 7975 | C | THR | C | 581 | 22.356 | 102.267 | 30.958 | 1.00 | 40.22 | 6 |
| ATOM | 7976 | O | THR | C | 581 | 22.575 | 101.222 | 30.359 | 1.00 | 41.67 | 8 |
| ATOM | 7977 | N | GLY | C | 582 | 21.814 | 103.319 | 30.356 | 1.00 | 61.44 | 7 |
| ATOM | 7978 | CA | GLY | C | 582 | 21.370 | 103.218 | 28.969 | 1.00 | 61.60 | 6 |
| ATOM | 7979 | C | GLY | C | 582 | 22.265 | 102.711 | 27.832 | 1.00 | 61.20 | 6 |
| ATOM | 7980 | O | GLY | C | 582 | 21.784 | 102.051 | 26.911 | 1.00 | 61.97 | 8 |
| ATOM | 7981 | N | LEU | C | 583 | 23.560 | 103.004 | 27.895 | 1.00 | 33.10 | 7 |
| ATOM | 7982 | CA | LEU | C | 583 | 24.509 | 102.643 | 26.841 | 1.00 | 29.69 | 6 |
| ATOM | 7983 | CB | LEU | C | 583 | 25.930 | 102.960 | 27.315 | 1.00 | 14.70 | 6 |
| ATOM | 7984 | CG | LEU | C | 583 | 26.222 | 104.458 | 27.262 | 1.00 | 13.87 | 6 |
| ATOM | 7985 | CD1 | LEU | C | 583 | 27.584 | 104.769 | 27.812 | 1.00 | 13.87 | 6 |
| ATOM | 7986 | CD2 | LEU | C | 583 | 26.118 | 104.910 | 25.826 | 1.00 | 13.87 | 6 |
| ATOM | 7987 | C | LEU | C | 583 | 24.499 | 101.267 | 26.203 | 1.00 | 29.36 | 6 |
| ATOM | 7988 | O | LEU | C | 583 | 24.790 | 101.137 | 25.019 | 1.00 | 29.00 | 8 |
| ATOM | 7989 | N | GLU | C | 584 | 24.164 | 100.244 | 26.973 | 1.00 | 32.86 | 7 |
| ATOM | 7990 | CA | GLU | C | 584 | 24.166 | 98.870 | 26.479 | 1.00 | 35.09 | 6 |
| ATOM | 7991 | CB | GLU | C | 584 | 23.655 | 97.992 | 27.584 | 1.00 | 21.12 | 6 |
| ATOM | 7992 | CG | GLU | C | 584 | 24.063 | 98.634 | 28.854 | 1.00 | 20.91 | 6 |
| ATOM | 7993 | CD | GLU | C | 584 | 23.532 | 97.953 | 30.050 | 1.00 | 21.97 | 6 |
| ATOM | 7994 | OE1 | GLU | C | 584 | 22.324 | 97.628 | 30.031 | 1.00 | 20.71 | 8 |
| ATOM | 7995 | OE2 | GLU | C | 584 | 24.323 | 97.760 | 31.011 | 1.00 | 23.30 | 8 |
| ATOM | 7996 | C | GLU | C | 584 | 23.360 | 98.679 | 25.209 | 1.00 | 38.29 | 6 |
| ATOM | 7997 | O | GLU | C | 584 | 23.690 | 97.829 | 24.376 | 1.00 | 39.52 | 8 |
| ATOM | 7998 | N | GLU | C | 585 | 22.314 | 99.487 | 25.051 | 1.00 | 55.36 | 7 |
| ATOM | 7999 | CA | GLU | C | 585 | 21.450 | 99.423 | 23.874 | 1.00 | 58.23 | 6 |
| ATOM | 8000 | CB | GLU | C | 585 | 20.259 | 100.389 | 24.024 | 1.00 | 85.92 | 6 |
| ATOM | 8001 | CG | GLU | C | 585 | 19.548 | 100.353 | 25.382 | 1.00 | 89.81 | 6 |
| ATOM | 8002 | CD | GLU | C | 585 | 18.416 | 101.375 | 25.507 | 1.00 | 92.09 | 6 |
| ATOM | 8003 | OE1 | GLU | C | 585 | 18.688 | 102.594 | 25.505 | 1.00 | 94.24 | 8 |
| ATOM | 8004 | OE2 | GLU | C | 585 | 17.247 | 100.956 | 25.612 | 1.00 | 93.62 | 8 |
| ATOM | 8005 | C | GLU | C | 585 | 22.265 | 99.842 | 22.659 | 1.00 | 58.83 | 6 |
| ATOM | 8006 | O | GLU | C | 585 | 21.726 | 99.937 | 21.561 | 1.00 | 58.39 | 8 |
| ATOM | 8007 | N | ARG | C | 586 | 23.560 | 100.082 | 22.860 | 1.00 | 62.56 | 7 |
| ATOM | 8008 | CA | ARG | C | 586 | 24.427 | 100.544 | 21.784 | 1.00 | 63.44 | 6 |
| ATOM | 8009 | CB | ARG | C | 586 | 24.666 | 102.041 | 21.947 | 1.00 | 43.28 | 6 |
| ATOM | 8010 | CG | ARG | C | 586 | 25.247 | 102.691 | 20.730 | 1.00 | 45.77 | 6 |
| ATOM | 8011 | CD | ARG | C | 586 | 25.462 | 104.173 | 20.924 | 1.00 | 49.63 | 6 |
| ATOM | 8012 | NE | ARG | C | 586 | 25.643 | 104.814 | 19.629 | 1.00 | 54.25 | 7 |
| ATOM | 8013 | CZ | ARG | C | 586 | 24.647 | 105.069 | 18.789 | 1.00 | 57.52 | 6 |
| ATOM | 8014 | NH1 | ARG | C | 586 | 23.405 | 104.751 | 19.122 | 1.00 | 58.30 | 7 |
| ATOM | 8015 | NH2 | ARG | C | 586 | 24.895 | 105.604 | 17.601 | 1.00 | 59.21 | 7 |
| ATOM | 8016 | C | ARG | C | 586 | 25.778 | 99.854 | 21.619 | 1.00 | 64.38 | 6 |
| ATOM | 8017 | O | ARG | C | 586 | 26.085 | 99.318 | 20.557 | 1.00 | 65.33 | 8 |
| ATOM | 8018 | N | VAL | C | 587 | 26.605 | 99.895 | 22.654 | 1.00 | 33.31 | 7 |
| ATOM | 8019 | CA | VAL | C | 587 | 27.922 | 99.274 | 22.584 | 1.00 | 33.17 | 6 |
| ATOM | 8020 | CB | VAL | C | 587 | 28.479 | 99.026 | 23.991 | 1.00 | 27.55 | 6 |
| ATOM | 8021 | CG1 | VAL | C | 587 | 29.711 | 98.138 | 23.930 | 1.00 | 26.75 | 6 |
| ATOM | 8022 | CG2 | VAL | C | 587 | 28.800 | 100.379 | 24.643 | 1.00 | 26.75 | 6 |
| ATOM | 8023 | C | VAL | C | 587 | 27.919 | 97.968 | 21.801 | 1.00 | 34.49 | 6 |
| ATOM | 8024 | O | VAL | C | 587 | 28.942 | 97.568 | 21.268 | 1.00 | 34.65 | 8 |
| ATOM | 8025 | N | VAL | C | 588 | 26.771 | 97.308 | 21.725 | 1.00 | 43.57 | 7 |
| ATOM | 8026 | CA | VAL | C | 588 | 26.665 | 96.056 | 20.988 | 1.00 | 45.37 | 6 |
| ATOM | 8027 | CB | VAL | C | 588 | 26.054 | 94.956 | 21.871 | 1.00 | 42.64 | 6 |
| ATOM | 8028 | CG1 | VAL | C | 588 | 25.582 | 93.762 | 21.025 | 1.00 | 42.85 | 6 |
| ATOM | 8029 | CG2 | VAL | C | 588 | 27.112 | 94.492 | 22.862 | 1.00 | 45.43 | 6 |
| ATOM | 8030 | C | VAL | C | 588 | 25.810 | 96.294 | 19.770 | 1.00 | 47.23 | 6 |
| ATOM | 8031 | O | VAL | C | 588 | 24.911 | 95.522 | 19.434 | 1.00 | 49.80 | 8 |
| ATOM | 8032 | N | ARG | C | 589 | 26.098 | 97.389 | 19.097 | 1.00 | 27.94 | 7 |
| ATOM | 8033 | CA | ARG | C | 589 | 25.343 | 97.725 | 17.922 | 1.00 | 27.95 | 6 |
| ATOM | 8034 | CB | ARG | C | 589 | 24.034 | 98.400 | 18.315 | 1.00 | 84.19 | 6 |
| ATOM | 8035 | CG | ARG | C | 589 | 22.853 | 97.457 | 18.223 | 1.00 | 86.48 | 6 |
| ATOM | 8036 | CD | ARG | C | 589 | 22.514 | 97.279 | 16.771 | 1.00 | 88.53 | 6 |
| ATOM | 8037 | NE | ARG | C | 589 | 21.700 | 96.107 | 16.502 | 1.00 | 90.71 | 7 |
| ATOM | 8038 | CZ | ARG | C | 589 | 21.102 | 95.897 | 15.336 | 1.00 | 91.23 | 6 |
| ATOM | 8039 | NH1 | ARG | C | 589 | 21.233 | 96.785 | 14.360 | 1.00 | 90.22 | 7 |
| ATOM | 8040 | NH2 | ARG | C | 589 | 20.384 | 94.802 | 15.140 | 1.00 | 92.10 | 7 |
| ATOM | 8041 | C | ARG | C | 589 | 26.193 | 98.605 | 17.073 | 1.00 | 26.89 | 6 |
| ATOM | 8042 | O | ARG | C | 589 | 25.901 | 98.817 | 15.912 | 1.00 | 26.93 | 8 |
| ATOM | 8043 | N | ASP | C | 590 | 27.249 | 99.139 | 17.662 | 1.00 | 56.02 | 7 |
| ATOM | 8044 | CA | ASP | C | 590 | 28.183 | 99.930 | 16.888 | 1.00 | 56.65 | 6 |
| ATOM | 8045 | CB | ASP | C | 590 | 28.612 | 101.177 | 17.658 | 1.00 | 44.75 | 6 |
| ATOM | 8046 | CG | ASP | C | 590 | 27.608 | 102.311 | 17.526 | 1.00 | 44.37 | 6 |
| ATOM | 8047 | OD1 | ASP | C | 590 | 27.206 | 102.624 | 16.389 | 1.00 | 44.16 | 8 |
| ATOM | 8048 | OD2 | ASP | C | 590 | 27.221 | 102.896 | 18.552 | 1.00 | 43.91 | 8 |
| ATOM | 8049 | C | ASP | C | 590 | 29.341 | 98.947 | 16.689 | 1.00 | 57.18 | 6 |
| ATOM | 8050 | O | ASP | C | 590 | 30.247 | 99.150 | 15.876 | 1.00 | 58.77 | 8 |
| ATOM | 8051 | N | SER | C | 591 | 29.245 | 97.846 | 17.428 | 1.00 | 57.47 | 7 |
| ATOM | 8052 | CA | SER | C | 591 | 30.215 | 96.760 | 17.403 | 1.00 | 55.46 | 6 |
| ATOM | 8053 | CB | SER | C | 591 | 30.044 | 95.898 | 18.651 | 1.00 | 35.19 | 6 |
| ATOM | 8054 | OG | SER | C | 591 | 28.749 | 95.324 | 18.702 | 1.00 | 33.80 | 8 |
| ATOM | 8055 | C | SER | C | 591 | 29.982 | 95.893 | 16.182 | 1.00 | 54.43 | 6 |
| ATOM | 8056 | O | SER | C | 591 | 30.718 | 94.940 | 15.924 | 1.00 | 54.69 | 8 |
| ATOM | 8057 | N | LEU | C | 592 | 28.932 | 96.235 | 15.450 | 1.00 | 45.12 | 7 |
| ATOM | 8058 | CA | LEU | C | 592 | 28.528 | 95.507 | 14.265 | 1.00 | 43.08 | 6 |
| ATOM | 8059 | CB | LEU | C | 592 | 29.379 | 95.919 | 13.068 | 1.00 | 67.56 | 6 |
| ATOM | 8060 | CG | LEU | C | 592 | 28.960 | 97.287 | 12.526 | 1.00 | 68.37 | 6 |
| ATOM | 8061 | CD1 | LEU | C | 592 | 29.673 | 97.540 | 11.217 | 1.00 | 68.27 | 6 |
| ATOM | 8062 | CD2 | LEU | C | 592 | 27.441 | 97.336 | 12.314 | 1.00 | 67.42 | 6 |
| ATOM | 8063 | C | LEU | C | 592 | 28.586 | 94.011 | 14.474 | 1.00 | 42.04 | 6 |
| ATOM | 8064 | O | LEU | C | 592 | 28.633 | 93.255 | 13.519 | 1.00 | 41.72 | 8 |
| ATOM | 8065 | N | ALA | C | 593 | 28.562 | 93.589 | 15.732 | 1.00 | 25.75 | 7 |
| ATOM | 8066 | CA | ALA | C | 593 | 28.621 | 92.169 | 16.053 | 1.00 | 26.87 | 6 |
| ATOM | 8067 | CB | ALA | C | 593 | 29.038 | 91.982 | 17.504 | 1.00 | 31.71 | 6 |
| ATOM | 8068 | C | ALA | C | 593 | 27.277 | 91.481 | 15.792 | 1.00 | 28.06 | 6 |
| ATOM | 8069 | O | ALA | C | 593 | 27.219 | 90.258 | 15.622 | 1.00 | 28.12 | 8 |
| ATOM | 8070 | N | ALA | C | 594 | 26.206 | 92.275 | 15.762 | 1.00 | 41.56 | 7 |
| ATOM | 8071 | CA | ALA | C | 594 | 24.861 | 91.768 | 15.520 | 1.00 | 43.85 | 6 |
| ATOM | 8072 | CB | ALA | C | 594 | 23.942 | 92.183 | 16.625 | 1.00 | 21.34 | 6 |
| ATOM | 8073 | C | ALA | C | 594 | 24.383 | 92.336 | 14.200 | 1.00 | 46.63 | 6 |
| ATOM | 8074 | O | ALA | C | 594 | 24.893 | 93.355 | 13.735 | 1.00 | 47.16 | 8 |
| ATOM | 8075 | N | LEU | C | 595 | 23.389 | 91.680 | 13.613 | 1.00 | 67.64 | 7 |
| ATOM | 8076 | CA | LEU | C | 595 | 22.848 | 92.063 | 12.312 | 1.00 | 71.12 | 6 |
| ATOM | 8077 | CB | LEU | C | 595 | 22.225 | 90.828 | 11.667 | 1.00 | 45.48 | 6 |
| ATOM | 8078 | CG | LEU | C | 595 | 21.863 | 90.819 | 10.187 | 1.00 | 45.09 | 6 |
| ATOM | 8079 | CD1 | LEU | C | 595 | 23.041 | 91.241 | 9.342 | 1.00 | 45.90 | 6 |
| ATOM | 8080 | CD2 | LEU | C | 595 | 21.422 | 89.416 | 9.817 | 1.00 | 45.26 | 6 |
| ATOM | 8081 | C | LEU | C | 595 | 21.837 | 93.202 | 12.363 | 1.00 | 73.93 | 6 |
| ATOM | 8082 | O | LEU | C | 595 | 20.920 | 93.194 | 13.179 | 1.00 | 74.05 | 8 |
| ATOM | 8083 | N | TYR | C | 596 | 22.008 | 94.177 | 11.474 | 1.00 | 99.78 | 7 |
| ATOM | 8084 | CA | TYR | C | 596 | 21.118 | 95.335 | 11.426 | 1.00 | 103.58 | 6 |
| ATOM | 8085 | CB | TYR | C | 596 | 21.930 | 96.622 | 11.654 | 1.00 | 208.87 | 6 |
| ATOM | 8086 | CG | TYR | C | 596 | 22.964 | 96.938 | 10.591 | 1.00 | 208.87 | 6 |
| ATOM | 8087 | CD1 | TYR | C | 596 | 22.606 | 97.595 | 9.413 | 1.00 | 208.87 | 6 |
| ATOM | 8088 | CE1 | TYR | C | 596 | 23.549 | 97.880 | 8.433 | 1.00 | 208.87 | 6 |
| ATOM | 8089 | CD2 | TYR | C | 596 | 24.298 | 96.572 | 10.761 | 1.00 | 208.87 | 6 |
| ATOM | 8090 | CE2 | TYR | C | 596 | 25.250 | 96.852 | 9.786 | 1.00 | 208.87 | 6 |
| ATOM | 8091 | CZ | TYR | C | 596 | 24.868 | 97.505 | 8.626 | 1.00 | 208.87 | 6 |
| ATOM | 8092 | OH | TYR | C | 596 | 25.808 | 97.779 | 7.662 | 1.00 | 208.87 | 8 |
| ATOM | 8093 | C | TYR | C | 596 | 20.301 | 95.450 | 10.135 | 1.00 | 103.76 | 6 |
| ATOM | 8094 | O | TYR | C | 596 | 20.854 | 95.557 | 9.038 | 1.00 | 104.74 | 8 |
| ATOM | 8095 | N | ALA | C | 597 | 18.979 | 95.433 | 10.277 | 1.00 | 56.72 | 7 |
| ATOM | 8096 | CA | ALA | C | 597 | 18.088 | 95.542 | 9.131 | 1.00 | 57.00 | 6 |
| ATOM | 8097 | CB | ALA | C | 597 | 16.657 | 95.428 | 9.581 | 1.00 | 13.87 | 6 |
| ATOM | 8098 | C | ALA | C | 597 | 18.308 | 96.873 | 8.431 | 1.00 | 57.65 | 6 |
| ATOM | 8099 | O | ALA | C | 597 | 18.686 | 97.850 | 9.067 | 1.00 | 56.88 | 8 |
| ATOM | 8100 | N | GLU | C | 598 | 18.066 | 96.902 | 7.122 | 1.00 | 73.43 | 7 |
| ATOM | 8101 | CA | GLU | C | 598 | 18.236 | 98.111 | 6.312 | 1.00 | 74.93 | 6 |
| ATOM | 8102 | CB | GLU | C | 598 | 18.650 | 97.742 | 4.880 | 1.00 | 128.78 | 6 |
| ATOM | 8103 | CG | GLU | C | 598 | 20.145 | 97.538 | 4.657 | 1.00 | 132.24 | 6 |
| ATOM | 8104 | CD | GLU | C | 598 | 20.466 | 97.165 | 3.217 | 1.00 | 133.80 | 6 |
| ATOM | 8105 | OE1 | GLU | C | 598 | 20.025 | 97.894 | 2.302 | 1.00 | 134.77 | 8 |
| ATOM | 8106 | OE2 | GLU | C | 598 | 21.160 | 96.148 | 2.994 | 1.00 | 134.91 | 8 |
| ATOM | 8107 | C | GLU | C | 598 | 16.972 | 98.968 | 6.251 | 1.00 | 74.63 | 6 |
| ATOM | 8108 | O | GLU | C | 598 | 16.477 | 99.454 | 7.270 | 1.00 | 74.33 | 8 |
| ATOM | 8109 | N | GLU | C | 599 | 16.458 | 99.155 | 5.042 | 1.00 | 63.28 | 7 |
| ATOM | 8110 | CA | GLU | C | 599 | 15.258 | 99.952 | 4.852 | 1.00 | 64.05 | 6 |
| ATOM | 8111 | CB | GLU | C | 599 | 15.089 | 100.333 | 3.372 | 1.00 | 85.98 | 6 |
| ATOM | 8112 | CG | GLU | C | 599 | 16.244 | 101.120 | 2.754 | 1.00 | 86.67 | 6 |
| ATOM | 8113 | CD | GLU | C | 599 | 17.399 | 100.235 | 2.307 | 1.00 | 87.36 | 6 |
| ATOM | 8114 | OE1 | GLU | C | 599 | 17.186 | 99.349 | 1.447 | 1.00 | 87.49 | 8 |
| ATOM | 8115 | OE2 | GLU | C | 599 | 18.525 | 100.430 | 2.812 | 1.00 | 87.70 | 8 |
| ATOM | 8116 | C | GLU | C | 599 | 14.011 | 99.205 | 5.331 | 1.00 | 64.02 | 6 |
| ATOM | 8117 | O | GLU | C | 599 | 13.984 | 97.973 | 5.380 | 1.00 | 62.83 | 8 |
| ATOM | 8118 | N | ASP | C | 600 | 12.989 | 99.981 | 5.681 | 1.00 | 122.82 | 7 |
| ATOM | 8119 | CA | ASP | C | 600 | 11.707 | 99.471 | 6.150 | 1.00 | 123.27 | 6 |
| ATOM | 8120 | CB | ASP | C | 600 | 10.675 | 99.549 | 5.019 | 1.00 | 143.28 | 6 |
| ATOM | 8121 | CG | ASP | C | 600 | 9.803 | 100.795 | 5.105 | 1.00 | 144.39 | 6 |
| ATOM | 8122 | OD1 | ASP | C | 600 | 9.093 | 100.952 | 6.121 | 1.00 | 144.36 | 8 |
| ATOM | 8123 | OD2 | ASP | C | 600 | 9.822 | 101.612 | 4.159 | 1.00 | 145.27 | 8 |
| ATOM | 8124 | C | ASP | C | 600 | 11.710 | 98.067 | 6.743 | 1.00 | 123.01 | 6 |
| ATOM | 8125 | O | ASP | C | 600 | 12.702 | 97.622 | 7.319 | 1.00 | 123.81 | 8 |
| ATOM | 8126 | N | GLY | C | 601 | 10.584 | 97.375 | 6.596 | 1.00 | 71.19 | 7 |
| ATOM | 8127 | CA | GLY | C | 601 | 10.450 | 96.037 | 7.143 | 1.00 | 70.09 | 6 |
| ATOM | 8128 | C | GLY | C | 601 | 10.145 | 94.930 | 6.159 | 1.00 | 69.28 | 6 |
| ATOM | 8129 | O | GLY | C | 601 | 10.481 | 95.033 | 4.984 | 1.00 | 68.71 | 8 |
| ATOM | 8130 | N | ALA | C | 602 | 9.496 | 93.880 | 6.664 | 1.00 | 152.74 | 7 |
| ATOM | 8131 | CA | ALA | C | 602 | 9.135 | 92.674 | 5.913 | 1.00 | 152.02 | 6 |
| ATOM | 8132 | CB | ALA | C | 602 | 9.206 | 92.910 | 4.404 | 1.00 | 75.08 | 6 |
| ATOM | 8133 | C | ALA | C | 602 | 10.147 | 91.612 | 6.334 | 1.00 | 151.47 | 6 |
| ATOM | 8134 | O | ALA | C | 602 | 10.923 | 91.839 | 7.256 | 1.00 | 152.79 | 8 |
| ATOM | 8135 | N | VAL | C | 603 | 10.142 | 90.457 | 5.676 | 1.00 | 85.64 | 7 |
| ATOM | 8136 | CA | VAL | C | 603 | 11.086 | 89.397 | 6.023 | 1.00 | 83.57 | 6 |
| ATOM | 8137 | CB | VAL | C | 603 | 10.783 | 88.811 | 7.427 | 1.00 | 62.32 | 6 |
| ATOM | 8138 | CG1 | VAL | C | 603 | 11.673 | 89.475 | 8.471 | 1.00 | 61.84 | 6 |
| ATOM | 8139 | CG2 | VAL | C | 603 | 9.318 | 89.026 | 7.779 | 1.00 | 62.26 | 6 |
| ATOM | 8140 | C | VAL | C | 603 | 11.139 | 88.258 | 5.001 | 1.00 | 82.75 | 6 |
| ATOM | 8141 | O | VAL | C | 603 | 11.578 | 88.473 | 3.869 | 1.00 | 83.33 | 8 |
| ATOM | 8142 | N | VAL | C | 604 | 10.707 | 87.061 | 5.418 | 1.00 | 52.44 | 7 |
| ATOM | 8143 | CA | VAL | C | 604 | 10.677 | 85.827 | 4.600 | 1.00 | 51.13 | 6 |
| ATOM | 8144 | CB | VAL | C | 604 | 11.256 | 86.016 | 3.177 | 1.00 | 79.65 | 6 |
| ATOM | 8145 | CG1 | VAL | C | 604 | 11.554 | 84.655 | 2.573 | 1.00 | 81.30 | 6 |
| ATOM | 8146 | CG2 | VAL | C | 604 | 10.267 | 86.763 | 2.289 | 1.00 | 79.72 | 6 |
| ATOM | 8147 | C | VAL | C | 604 | 11.465 | 84.687 | 5.254 | 1.00 | 50.52 | 6 |
| ATOM | 8148 | O | VAL | C | 604 | 10.923 | 83.620 | 5.533 | 1.00 | 49.82 | 8 |
| ATOM | 8149 | N | LYS | C | 605 | 12.755 | 84.923 | 5.450 | 1.00 | 60.13 | 7 |
| ATOM | 8150 | CA | LYS | C | 605 | 13.659 | 83.973 | 6.088 | 1.00 | 59.49 | 6 |
| ATOM | 8151 | CB | LYS | C | 605 | 13.505 | 84.065 | 7.604 | 1.00 | 56.16 | 6 |
| ATOM | 8152 | CG | LYS | C | 605 | 14.711 | 83.573 | 8.371 | 1.00 | 54.72 | 6 |
| ATOM | 8153 | CD | LYS | C | 605 | 14.389 | 83.349 | 9.825 | 1.00 | 53.41 | 6 |
| ATOM | 8154 | CE | LYS | C | 605 | 13.736 | 84.540 | 10.464 | 1.00 | 53.12 | 6 |
| ATOM | 8155 | NZ | LYS | C | 605 | 13.411 | 84.236 | 11.885 | 1.00 | 54.21 | 7 |
| ATOM | 8156 | C | LYS | C | 605 | 13.539 | 82.508 | 5.679 | 1.00 | 60.19 | 6 |
| ATOM | 8157 | O | LYS | C | 605 | 13.682 | 82.149 | 4.506 | 1.00 | 60.29 | 8 |
| ATOM | 8158 | N | VAL | C | 606 | 13.298 | 81.686 | 6.700 | 1.00 | 103.81 | 7 |
| ATOM | 8159 | CA | VAL | C | 606 | 13.147 | 80.231 | 6.641 | 1.00 | 104.50 | 6 |
| ATOM | 8160 | CB | VAL | C | 606 | 13.940 | 79.568 | 5.496 | 1.00 | 64.26 | 6 |
| ATOM | 8161 | CG1 | VAL | C | 606 | 15.419 | 79.955 | 5.579 | 1.00 | 64.08 | 6 |
| ATOM | 8162 | CG2 | VAL | C | 606 | 13.787 | 78.043 | 5.584 | 1.00 | 62.80 | 6 |
| ATOM | 8163 | C | VAL | C | 606 | 13.767 | 79.762 | 7.941 | 1.00 | 105.07 | 6 |
| ATOM | 8164 | O | VAL | C | 606 | 13.624 | 78.610 | 8.342 | 1.00 | 105.81 | 8 |
| ATOM | 8165 | N | ASP | C | 607 | 14.472 | 80.690 | 8.579 | 1.00 | 52.06 | 7 |
| ATOM | 8166 | CA | ASP | C | 607 | 15.158 | 80.455 | 9.843 | 1.00 | 53.06 | 6 |
| ATOM | 8167 | CB | ASP | C | 607 | 14.166 | 79.941 | 10.877 | 1.00 | 136.87 | 6 |
| ATOM | 8168 | CG | ASP | C | 607 | 13.510 | 81.063 | 11.652 | 1.00 | 139.08 | 6 |
| ATOM | 8169 | OD1 | ASP | C | 607 | 14.252 | 81.866 | 12.259 | 1.00 | 139.35 | 8 |
| ATOM | 8170 | OD2 | ASP | C | 607 | 12.264 | 81.142 | 11.660 | 1.00 | 140.44 | 8 |
| ATOM | 8171 | C | ASP | C | 607 | 16.382 | 79.547 | 9.799 | 1.00 | 52.92 | 6 |
| ATOM | 8172 | O | ASP | C | 607 | 16.487 | 78.641 | 8.962 | 1.00 | 53.19 | 8 |
| ATOM | 8173 | N | GLY | C | 608 | 17.322 | 79.816 | 10.698 | 1.00 | 114.79 | 7 |
| ATOM | 8174 | CA | GLY | C | 608 | 18.521 | 79.007 | 10.762 | 1.00 | 114.98 | 6 |
| ATOM | 8175 | C | GLY | C | 608 | 19.850 | 79.714 | 10.576 | 1.00 | 114.21 | 6 |
| ATOM | 8176 | O | GLY | C | 608 | 20.257 | 80.544 | 11.393 | 1.00 | 113.43 | 8 |
| ATOM | 8177 | N | THR | C | 609 | 20.524 | 79.373 | 9.484 | 1.00 | 77.25 | 7 |
| ATOM | 8178 | CA | THR | C | 609 | 21.831 | 79.922 | 9.176 | 1.00 | 77.03 | 6 |
| ATOM | 8179 | CB | THR | C | 609 | 22.696 | 78.855 | 8.476 | 1.00 | 40.34 | 6 |
| ATOM | 8180 | OG1 | THR | C | 609 | 24.089 | 79.185 | 8.604 | 1.00 | 38.50 | 8 |
| ATOM | 8181 | CG2 | THR | C | 609 | 22.318 | 78.774 | 7.004 | 1.00 | 39.60 | 6 |
| ATOM | 8182 | C | THR | C | 609 | 21.780 | 81.178 | 8.305 | 1.00 | 78.19 | 6 |
| ATOM | 8183 | O | THR | C | 609 | 22.743 | 81.485 | 7.599 | 1.00 | 78.53 | 8 |
| ATOM | 8184 | N | ALA | C | 610 | 20.665 | 81.903 | 8.351 | 1.00 | 102.56 | 7 |
| ATOM | 8185 | CA | ALA | C | 610 | 20.543 | 83.133 | 7.573 | 1.00 | 103.01 | 6 |
| ATOM | 8186 | CB | ALA | C | 610 | 20.996 | 82.888 | 6.137 | 1.00 | 98.76 | 6 |
| ATOM | 8187 | C | ALA | C | 610 | 19.150 | 83.751 | 7.560 | 1.00 | 103.30 | 6 |
| ATOM | 8188 | O | ALA | C | 610 | 18.204 | 83.121 | 7.097 | 1.00 | 104.01 | 8 |
| ATOM | 8189 | N | ILE | C | 611 | 19.016 | 84.975 | 8.073 | 1.00 | 78.66 | 7 |
| ATOM | 8190 | CA | ILE | C | 611 | 17.719 | 85.644 | 8.038 | 1.00 | 78.67 | 6 |
| ATOM | 8191 | CB | ILE | C | 611 | 17.710 | 87.019 | 8.778 | 1.00 | 48.67 | 6 |
| ATOM | 8192 | CG2 | ILE | C | 611 | 16.859 | 87.998 | 8.017 | 1.00 | 49.38 | 6 |
| ATOM | 8193 | CG1 | ILE | C | 611 | 17.076 | 86.907 | 10.168 | 1.00 | 47.17 | 6 |
| ATOM | 8194 | CD1 | ILE | C | 611 | 17.863 | 86.119 | 11.149 | 1.00 | 46.07 | 6 |
| ATOM | 8195 | C | ILE | C | 611 | 17.587 | 85.898 | 6.549 | 1.00 | 79.77 | 6 |
| ATOM | 8196 | O | ILE | C | 611 | 18.400 | 86.615 | 5.970 | 1.00 | 79.64 | 8 |
| ATOM | 8197 | N | ALA | C | 612 | 16.588 | 85.291 | 5.920 | 1.00 | 65.93 | 7 |
| ATOM | 8198 | CA | ALA | C | 612 | 16.411 | 85.466 | 4.489 | 1.00 | 67.35 | 6 |
| ATOM | 8199 | CB | ALA | C | 612 | 15.664 | 84.279 | 3.886 | 1.00 | 127.77 | 6 |
| ATOM | 8200 | C | ALA | C | 612 | 15.697 | 86.754 | 4.164 | 1.00 | 68.82 | 6 |
| ATOM | 8201 | O | ALA | C | 612 | 14.474 | 86.852 | 4.230 | 1.00 | 69.13 | 8 |
| ATOM | 8202 | N | VAL | C | 613 | 16.500 | 87.752 | 3.839 | 1.00 | 87.03 | 7 |
| ATOM | 8203 | CA | VAL | C | 613 | 16.011 | 89.058 | 3.455 | 1.00 | 88.75 | 6 |
| ATOM | 8204 | CB | VAL | C | 613 | 15.435 | 89.009 | 2.035 | 1.00 | 191.61 | 6 |
| ATOM | 8205 | CG1 | VAL | C | 613 | 15.242 | 90.419 | 1.505 | 1.00 | 192.00 | 6 |
| ATOM | 8206 | CG2 | VAL | C | 613 | 16.348 | 88.188 | 1.136 | 1.00 | 192.66 | 6 |
| ATOM | 8207 | C | VAL | C | 613 | 14.974 | 89.743 | 4.336 | 1.00 | 89.29 | 6 |
| ATOM | 8208 | O | VAL | C | 613 | 14.395 | 89.173 | 5.260 | 1.00 | 89.25 | 8 |
| ATOM | 8209 | N | ARG | C | 614 | 14.778 | 91.005 | 3.999 | 1.00 | 112.69 | 7 |
| ATOM | 8210 | CA | ARG | C | 614 | 13.839 | 91.907 | 4.618 | 1.00 | 113.97 | 6 |
| ATOM | 8211 | CB | ARG | C | 614 | 14.631 | 93.031 | 5.297 | 1.00 | 66.66 | 6 |
| ATOM | 8212 | CG | ARG | C | 614 | 15.895 | 93.427 | 4.519 | 1.00 | 65.25 | 6 |
| ATOM | 8213 | CD | ARG | C | 614 | 16.886 | 94.240 | 5.340 | 1.00 | 64.64 | 6 |
| ATOM | 8214 | NE | ARG | C | 614 | 18.182 | 94.315 | 4.666 | 1.00 | 63.93 | 7 |
| ATOM | 8215 | CZ | ARG | C | 614 | 19.336 | 94.590 | 5.272 | 1.00 | 64.57 | 6 |
| ATOM | 8216 | NH1 | ARG | C | 614 | 19.369 | 94.822 | 6.573 | 1.00 | 65.21 | 7 |
| ATOM | 8217 | NH2 | ARG | C | 614 | 20.468 | 94.612 | 4.581 | 1.00 | 65.16 | 7 |
| ATOM | 8218 | C | ARG | C | 614 | 13.101 | 92.380 | 3.358 | 1.00 | 115.71 | 6 |
| ATOM | 8219 | O | ARG | C | 614 | 13.391 | 93.438 | 2.809 | 1.00 | 117.13 | 8 |
| ATOM | 8220 | N | ALA | C | 615 | 12.175 | 91.546 | 2.896 | 1.00 | 94.59 | 7 |
| ATOM | 8221 | CA | ALA | C | 615 | 11.384 | 91.755 | 1.677 | 1.00 | 94.60 | 6 |
| ATOM | 8222 | CB | ALA | C | 615 | 10.156 | 90.844 | 1.730 | 1.00 | 126.60 | 6 |
| ATOM | 8223 | C | ALA | C | 615 | 10.949 | 93.147 | 1.187 | 1.00 | 94.78 | 6 |
| ATOM | 8224 | O | ALA | C | 615 | 10.038 | 93.243 | 0.355 | 1.00 | 95.34 | 8 |
| ATOM | 8225 | N | ALA | C | 616 | 11.575 | 94.216 | 1.669 | 1.00 | 105.19 | 7 |
| ATOM | 8226 | CA | ALA | C | 616 | 11.209 | 95.562 | 1.227 | 1.00 | 103.90 | 6 |
| ATOM | 8227 | CB | ALA | C | 616 | 11.425 | 96.566 | 2.354 | 1.00 | 115.14 | 6 |
| ATOM | 8228 | C | ALA | C | 616 | 12.055 | 95.942 | 0.016 | 1.00 | 103.39 | 6 |
| ATOM | 8229 | O | ALA | C | 616 | 13.279 | 96.033 | 0.113 | 1.00 | 103.74 | 8 |
| ATOM | 8230 | N | ALA | C | 617 | 11.394 | 96.154 | −1.122 | 1.00 | 152.23 | 7 |
| ATOM | 8231 | CA | ALA | C | 617 | 12.063 | 96.517 | −2.375 | 1.00 | 150.98 | 6 |
| ATOM | 8232 | CB | ALA | C | 617 | 12.968 | 97.740 | −2.159 | 1.00 | 105.32 | 6 |
| ATOM | 8233 | C | ALA | C | 617 | 12.871 | 95.345 | −2.949 | 1.00 | 149.51 | 6 |
| ATOM | 8234 | O | ALA | C | 617 | 12.300 | 94.397 | −3.490 | 1.00 | 150.02 | 8 |
| ATOM | 8235 | N | GLY | C | 618 | 14.195 | 95.418 | −2.840 | 1.00 | 105.94 | 7 |
| ATOM | 8236 | CA | GLY | C | 618 | 15.042 | 94.352 | −3.348 | 1.00 | 103.81 | 6 |
| ATOM | 8237 | C | GLY | C | 618 | 15.018 | 93.170 | −2.401 | 1.00 | 102.70 | 6 |
| ATOM | 8238 | O | GLY | C | 618 | 14.029 | 92.962 | −1.701 | 1.00 | 103.42 | 8 |
| ATOM | 8239 | N | ARG | C | 619 | 16.094 | 92.390 | −2.368 | 1.00 | 105.88 | 7 |
| ATOM | 8240 | CA | ARG | C | 619 | 16.151 | 91.238 | −1.470 | 1.00 | 103.75 | 6 |
| ATOM | 8241 | CB | ARG | C | 619 | 15.511 | 90.017 | −2.137 | 1.00 | 80.95 | 6 |
| ATOM | 8242 | CG | ARG | C | 619 | 14.646 | 89.240 | −1.196 | 1.00 | 82.19 | 6 |
| ATOM | 8243 | CD | ARG | C | 619 | 14.285 | 87.878 | −1.705 | 1.00 | 83.29 | 6 |
| ATOM | 8244 | NE | ARG | C | 619 | 14.236 | 86.920 | −0.604 | 1.00 | 84.96 | 7 |
| ATOM | 8245 | CZ | ARG | C | 619 | 13.425 | 85.865 | −0.543 | 1.00 | 86.74 | 6 |
| ATOM | 8246 | NH1 | ARG | C | 619 | 12.562 | 85.614 | −1.521 | 1.00 | 88.12 | 7 |
| ATOM | 8247 | NH2 | ARG | C | 619 | 13.494 | 85.040 | 0.494 | 1.00 | 88.66 | 7 |
| ATOM | 8248 | C | ARG | C | 619 | 17.592 | 90.916 | −1.052 | 1.00 | 102.23 | 6 |
| ATOM | 8249 | O | ARG | C | 619 | 18.514 | 91.018 | −1.865 | 1.00 | 103.00 | 8 |
| ATOM | 8250 | N | ALA | C | 620 | 17.792 | 90.529 | 0.209 | 1.00 | 46.28 | 7 |
| ATOM | 8251 | CA | ALA | C | 620 | 19.137 | 90.224 | 0.683 | 1.00 | 43.89 | 6 |
| ATOM | 8252 | CB | ALA | C | 620 | 19.802 | 91.498 | 1.151 | 1.00 | 55.43 | 6 |
| ATOM | 8253 | C | ALA | C | 620 | 19.216 | 89.184 | 1.781 | 1.00 | 42.30 | 6 |
| ATOM | 8254 | O | ALA | C | 620 | 19.118 | 89.516 | 2.947 | 1.00 | 41.57 | 8 |
| ATOM | 8255 | N | VAL | C | 621 | 19.402 | 87.925 | 1.409 | 1.00 | 53.94 | 7 |
| ATOM | 8256 | CA | VAL | C | 621 | 19.525 | 86.843 | 2.388 | 1.00 | 52.46 | 6 |
| ATOM | 8257 | CB | VAL | C | 621 | 19.751 | 85.469 | 1.685 | 1.00 | 104.37 | 6 |
| ATOM | 8258 | CG1 | VAL | C | 621 | 20.054 | 84.389 | 2.714 | 1.00 | 105.44 | 6 |
| ATOM | 8259 | CG2 | VAL | C | 621 | 18.521 | 85.086 | 0.877 | 1.00 | 104.85 | 6 |
| ATOM | 8260 | C | VAL | C | 621 | 20.730 | 87.155 | 3.284 | 1.00 | 51.08 | 6 |
| ATOM | 8261 | O | VAL | C | 621 | 21.841 | 87.383 | 2.799 | 1.00 | 51.01 | 8 |
| ATOM | 8262 | N | HIS | C | 623 | 20.511 | 87.165 | 4.590 | 1.00 | 53.04 | 7 |
| ATOM | 8263 | CA | HIS | C | 623 | 21.586 | 87.472 | 5.514 | 1.00 | 51.62 | 6 |
| ATOM | 8264 | CB | HIS | C | 623 | 21.097 | 88.456 | 6.565 | 1.00 | 49.20 | 6 |
| ATOM | 8265 | CG | HIS | C | 623 | 21.163 | 89.879 | 6.125 | 1.00 | 48.97 | 6 |
| ATOM | 8266 | CD2 | HIS | C | 623 | 21.846 | 90.468 | 5.117 | 1.00 | 48.83 | 6 |
| ATOM | 8267 | ND1 | HIS | C | 623 | 20.476 | 90.888 | 6.765 | 1.00 | 49.45 | 7 |
| ATOM | 8268 | CE1 | HIS | C | 623 | 20.731 | 92.037 | 6.169 | 1.00 | 49.14 | 6 |
| ATOM | 8269 | NE2 | HIS | C | 623 | 21.560 | 91.809 | 5.166 | 1.00 | 49.93 | 7 |
| ATOM | 8270 | C | HIS | C | 623 | 22.218 | 86.280 | 6.208 | 1.00 | 51.02 | 6 |
| ATOM | 8271 | O | HIS | C | 623 | 21.585 | 85.619 | 7.036 | 1.00 | 50.48 | 8 |
| ATOM | 8272 | N | PRO | C | 624 | 23.490 | 85.994 | 5.874 | 1.00 | 47.27 | 7 |
| ATOM | 8273 | CD | PRO | C | 624 | 24.227 | 86.607 | 4.753 | 1.00 | 73.09 | 6 |
| ATOM | 8274 | CA | PRO | C | 624 | 24.252 | 84.885 | 6.452 | 1.00 | 47.26 | 6 |
| ATOM | 8275 | CB | PRO | C | 624 | 25.545 | 84.888 | 5.636 | 1.00 | 72.75 | 6 |
| ATOM | 8276 | CG | PRO | C | 624 | 25.126 | 85.476 | 4.313 | 1.00 | 72.76 | 6 |
| ATOM | 8277 | C | PRO | C | 624 | 24.510 | 85.230 | 7.896 | 1.00 | 47.63 | 6 |
| ATOM | 8278 | O | PRO | C | 624 | 24.880 | 86.353 | 8.201 | 1.00 | 47.78 | 8 |
| ATOM | 8279 | N | LEU | C | 625 | 24.313 | 84.285 | 8.796 | 1.00 | 61.90 | 7 |
| ATOM | 8280 | CA | LEU | C | 625 | 24.561 | 84.590 | 10.191 | 1.00 | 62.50 | 6 |
| ATOM | 8281 | CB | LEU | C | 625 | 23.341 | 84.190 | 11.036 | 1.00 | 34.04 | 6 |
| ATOM | 8282 | CG | LEU | C | 625 | 22.009 | 84.938 | 10.871 | 1.00 | 32.77 | 6 |
| ATOM | 8283 | CD1 | LEU | C | 625 | 21.034 | 84.408 | 11.903 | 1.00 | 33.14 | 6 |
| ATOM | 8284 | CD2 | LEU | C | 625 | 22.180 | 86.435 | 11.069 | 1.00 | 33.40 | 6 |
| ATOM | 8285 | C | LEU | C | 625 | 25.833 | 83.914 | 10.719 | 1.00 | 62.74 | 6 |
| ATOM | 8286 | O | LEU | C | 625 | 25.827 | 82.711 | 10.981 | 1.00 | 63.85 | 8 |
| ATOM | 8287 | N | ARG | C | 626 | 26.916 | 84.686 | 10.865 | 1.00 | 42.15 | 7 |
| ATOM | 8288 | CA | ARG | C | 626 | 28.189 | 84.158 | 11.373 | 1.00 | 42.91 | 6 |
| ATOM | 8289 | CB | ARG | C | 626 | 29.079 | 85.300 | 11.884 | 1.00 | 116.16 | 6 |
| ATOM | 8290 | CG | ARG | C | 626 | 29.688 | 86.150 | 10.767 | 1.00 | 120.08 | 6 |
| ATOM | 8291 | CD | ARG | C | 626 | 30.320 | 87.465 | 11.266 | 1.00 | 122.23 | 6 |
| ATOM | 8292 | NE | ARG | C | 626 | 31.633 | 87.310 | 11.901 | 1.00 | 123.23 | 7 |
| ATOM | 8293 | CZ | ARG | C | 626 | 32.401 | 88.329 | 12.294 | 1.00 | 122.68 | 6 |
| ATOM | 8294 | NH1 | ARG | C | 626 | 31.995 | 89.581 | 12.120 | 1.00 | 123.21 | 7 |
| ATOM | 8295 | NH2 | ARG | C | 626 | 33.573 | 88.100 | 12.872 | 1.00 | 122.47 | 7 |
| ATOM | 8296 | C | ARG | C | 626 | 27.912 | 83.160 | 12.494 | 1.00 | 41.63 | 6 |
| ATOM | 8297 | O | ARG | C | 626 | 27.615 | 83.550 | 13.623 | 1.00 | 41.46 | 8 |
| ATOM | 8298 | N | ARG | C | 627 | 28.015 | 81.868 | 12.177 | 1.00 | 46.57 | 7 |
| ATOM | 8299 | CA | ARG | C | 627 | 27.712 | 80.817 | 13.149 | 1.00 | 45.53 | 6 |
| ATOM | 8300 | CB | ARG | C | 627 | 26.744 | 79.817 | 12.529 | 1.00 | 76.33 | 6 |
| ATOM | 8301 | CG | ARG | C | 627 | 25.840 | 79.169 | 13.559 | 1.00 | 77.92 | 6 |
| ATOM | 8302 | CD | ARG | C | 627 | 24.679 | 80.089 | 13.915 | 1.00 | 79.04 | 6 |
| ATOM | 8303 | NE | ARG | C | 627 | 24.070 | 79.737 | 15.193 | 1.00 | 79.30 | 7 |
| ATOM | 8304 | CZ | ARG | C | 627 | 22.786 | 79.914 | 15.474 | 1.00 | 79.43 | 6 |
| ATOM | 8305 | NH1 | ARG | C | 627 | 21.976 | 80.433 | 14.561 | 1.00 | 79.88 | 7 |
| ATOM | 8306 | NH2 | ARG | C | 627 | 22.314 | 79.579 | 16.667 | 1.00 | 78.53 | 7 |
| ATOM | 8307 | C | ARG | C | 627 | 28.865 | 80.040 | 13.786 | 1.00 | 44.52 | 6 |
| ATOM | 8308 | O | ARG | C | 627 | 29.764 | 79.547 | 13.101 | 1.00 | 44.13 | 8 |
| ATOM | 8309 | N | TYR | C | 628 | 28.786 | 79.908 | 15.110 | 1.00 | 37.59 | 7 |
| ATOM | 8310 | CA | TYR | C | 628 | 29.780 | 79.212 | 15.910 | 1.00 | 36.77 | 6 |
| ATOM | 8311 | CB | TYR | C | 628 | 29.414 | 77.740 | 16.056 | 1.00 | 63.07 | 6 |
| ATOM | 8312 | CG | TYR | C | 628 | 28.380 | 77.553 | 17.129 | 1.00 | 65.11 | 6 |
| ATOM | 8313 | CD1 | TYR | C | 628 | 27.139 | 78.197 | 17.047 | 1.00 | 65.45 | 6 |
| ATOM | 8314 | CE1 | TYR | C | 628 | 26.224 | 78.134 | 18.090 | 1.00 | 64.71 | 6 |
| ATOM | 8315 | CD2 | TYR | C | 628 | 28.673 | 76.829 | 18.281 | 1.00 | 65.64 | 6 |
| ATOM | 8316 | CE2 | TYR | C | 628 | 27.764 | 76.759 | 19.330 | 1.00 | 65.07 | 6 |
| ATOM | 8317 | CZ | TYR | C | 628 | 26.545 | 77.419 | 19.232 | 1.00 | 64.58 | 6 |
| ATOM | 8318 | OH | TYR | C | 628 | 25.678 | 77.393 | 20.301 | 1.00 | 64.00 | 8 |
| ATOM | 8319 | C | TYR | C | 628 | 31.151 | 79.370 | 15.334 | 1.00 | 36.13 | 6 |
| ATOM | 8320 | O | TYR | C | 628 | 31.915 | 78.418 | 15.229 | 1.00 | 35.88 | 8 |
| ATOM | 8321 | N | ALA | C | 629 | 31.448 | 80.609 | 14.972 | 1.00 | 40.49 | 7 |
| ATOM | 8322 | CA | ALA | C | 629 | 32.725 | 80.969 | 14.401 | 1.00 | 40.08 | 6 |
| ATOM | 8323 | CB | ALA | C | 629 | 32.791 | 82.459 | 14.204 | 1.00 | 56.04 | 6 |
| ATOM | 8324 | C | ALA | C | 629 | 33.851 | 80.530 | 15.303 | 1.00 | 40.02 | 6 |
| ATOM | 8325 | O | ALA | C | 629 | 33.881 | 79.400 | 15.802 | 1.00 | 39.39 | 8 |
| ATOM | 8326 | N | ARG | C | 630 | 34.789 | 81.447 | 15.496 | 1.00 | 24.76 | 7 |
| ATOM | 8327 | CA | ARG | C | 630 | 35.963 | 81.214 | 16.321 | 1.00 | 26.91 | 6 |
| ATOM | 8328 | CB | ARG | C | 630 | 37.053 | 80.508 | 15.504 | 1.00 | 70.75 | 6 |
| ATOM | 8329 | CG | ARG | C | 630 | 36.685 | 80.242 | 14.028 | 1.00 | 73.28 | 6 |
| ATOM | 8330 | CD | ARG | C | 630 | 35.829 | 78.982 | 13.841 | 1.00 | 74.43 | 6 |
| ATOM | 8331 | NE | ARG | C | 630 | 35.175 | 78.949 | 12.533 | 1.00 | 74.10 | 7 |
| ATOM | 8332 | CZ | ARG | C | 630 | 34.364 | 77.978 | 12.124 | 1.00 | 73.71 | 6 |
| ATOM | 8333 | NH1 | ARG | C | 630 | 34.102 | 76.947 | 12.920 | 1.00 | 72.93 | 7 |
| ATOM | 8334 | NH2 | ARG | C | 630 | 33.812 | 78.045 | 10.921 | 1.00 | 72.98 | 7 |
| ATOM | 8335 | C | ARG | C | 630 | 36.432 | 82.593 | 16.741 | 1.00 | 27.84 | 6 |
| ATOM | 8336 | O | ARG | C | 630 | 36.295 | 83.549 | 15.974 | 1.00 | 27.83 | 8 |
| ATOM | 8337 | N | SER | C | 631 | 36.981 | 82.719 | 17.943 | 1.00 | 75.23 | 7 |
| ATOM | 8338 | CA | SER | C | 631 | 37.426 | 84.034 | 18.402 | 1.00 | 77.41 | 6 |
| ATOM | 8339 | CB | SER | C | 631 | 36.795 | 84.345 | 19.750 | 1.00 | 102.85 | 6 |
| ATOM | 8340 | OG | SER | C | 631 | 35.385 | 84.196 | 19.684 | 1.00 | 104.52 | 8 |
| ATOM | 8341 | C | SER | C | 631 | 38.932 | 84.156 | 18.515 | 1.00 | 78.51 | 6 |
| ATOM | 8342 | O | SER | C | 631 | 39.631 | 83.163 | 18.662 | 1.00 | 79.56 | 8 |
| ATOM | 8343 | N | ASN | C | 632 | 39.430 | 85.382 | 18.434 | 1.00 | 67.55 | 7 |
| ATOM | 8344 | CA | ASN | C | 632 | 40.862 | 85.613 | 18.541 | 1.00 | 68.58 | 6 |
| ATOM | 8345 | CB | ASN | C | 632 | 41.176 | 87.101 | 18.501 | 1.00 | 55.31 | 6 |
| ATOM | 8346 | CG | ASN | C | 632 | 41.131 | 87.653 | 17.115 | 1.00 | 55.62 | 6 |
| ATOM | 8347 | OD1 | ASN | C | 632 | 42.019 | 87.403 | 16.312 | 1.00 | 51.87 | 8 |
| ATOM | 8348 | ND2 | ASN | C | 632 | 40.084 | 88.395 | 16.812 | 1.00 | 57.62 | 7 |
| ATOM | 8349 | C | ASN | C | 632 | 41.287 | 85.055 | 19.865 | 1.00 | 69.32 | 6 |
| ATOM | 8350 | O | ASN | C | 632 | 42.328 | 84.418 | 19.982 | 1.00 | 70.85 | 8 |
| ATOM | 8351 | N | GLN | C | 633 | 40.461 | 85.330 | 20.849 | 1.00 | 37.41 | 7 |
| ATOM | 8352 | CA | GLN | C | 633 | 40.687 | 84.815 | 22.193 | 1.00 | 38.28 | 6 |
| ATOM | 8353 | CB | GLN | C | 633 | 40.022 | 85.718 | 23.232 | 1.00 | 166.95 | 6 |
| ATOM | 8354 | CG | GLN | C | 633 | 39.552 | 87.056 | 22.683 | 1.00 | 169.97 | 6 |
| ATOM | 8355 | CD | GLN | C | 633 | 40.598 | 87.729 | 21.818 | 1.00 | 172.13 | 6 |
| ATOM | 8356 | OE1 | GLN | C | 633 | 40.302 | 88.683 | 21.097 | 1.00 | 172.88 | 8 |
| ATOM | 8357 | NE2 | GLN | C | 633 | 41.881 | 87.397 | 21.731 | 1.00 | 174.33 | 7 |
| ATOM | 8358 | C | GLN | C | 633 | 40.177 | 83.384 | 22.327 | 1.00 | 38.17 | 6 |
| ATOM | 8359 | O | GLN | C | 633 | 40.321 | 82.752 | 23.334 | 1.00 | 39.26 | 8 |
| ATOM | 8360 | N | GLY | C | 634 | 39.583 | 82.897 | 21.232 | 1.00 | 74.61 | 7 |
| ATOM | 8361 | CA | GLY | C | 634 | 39.065 | 81.542 | 21.268 | 1.00 | 74.34 | 6 |
| ATOM | 8362 | C | GLY | C | 634 | 37.733 | 81.524 | 21.979 | 1.00 | 74.02 | 6 |
| ATOM | 8363 | O | GLY | C | 634 | 37.279 | 80.497 | 22.474 | 1.00 | 74.35 | 8 |
| ATOM | 8364 | N | THR | C | 635 | 37.107 | 82.691 | 22.020 | 1.00 | 78.24 | 7 |
| ATOM | 8365 | CA | THR | C | 635 | 35.822 | 82.864 | 22.664 | 1.00 | 78.54 | 6 |
| ATOM | 8366 | CB | THR | C | 635 | 35.444 | 84.367 | 22.724 | 1.00 | 104.98 | 6 |
| ATOM | 8367 | OG1 | THR | C | 635 | 35.430 | 84.788 | 24.091 | 1.00 | 107.03 | 8 |
| ATOM | 8368 | CG2 | THR | C | 635 | 34.080 | 84.636 | 22.093 | 1.00 | 105.64 | 6 |
| ATOM | 8369 | C | THR | C | 635 | 34.706 | 82.069 | 22.005 | 1.00 | 78.33 | 6 |
| ATOM | 8370 | O | THR | C | 635 | 34.102 | 81.231 | 22.663 | 1.00 | 80.40 | 8 |
| ATOM | 8371 | N | ALA | C | 636 | 34.443 | 82.320 | 20.721 | 1.00 | 25.53 | 7 |
| ATOM | 8372 | CA | ALA | C | 636 | 33.374 | 81.644 | 19.986 | 1.00 | 23.68 | 6 |
| ATOM | 8373 | CB | ALA | C | 636 | 33.003 | 80.308 | 20.642 | 1.00 | 52.56 | 6 |
| ATOM | 8374 | C | ALA | C | 636 | 32.129 | 82.514 | 19.886 | 1.00 | 23.17 | 6 |
| ATOM | 8375 | O | ALA | C | 636 | 31.054 | 82.152 | 20.367 | 1.00 | 21.83 | 8 |
| ATOM | 8376 | N | PHE | C | 637 | 32.274 | 83.667 | 19.254 | 1.00 | 20.92 | 7 |
| ATOM | 8377 | CA | PHE | C | 637 | 31.150 | 84.576 | 19.078 | 1.00 | 20.85 | 6 |
| ATOM | 8378 | CB | PHE | C | 637 | 31.671 | 85.973 | 18.703 | 1.00 | 76.68 | 6 |
| ATOM | 8379 | CG | PHE | C | 637 | 31.455 | 86.331 | 17.268 | 1.00 | 76.26 | 6 |
| ATOM | 8380 | CD1 | PHE | C | 637 | 30.307 | 86.993 | 16.877 | 1.00 | 76.05 | 6 |
| ATOM | 8381 | CD2 | PHE | C | 637 | 32.365 | 85.950 | 16.298 | 1.00 | 76.17 | 6 |
| ATOM | 8382 | CE1 | PHE | C | 637 | 30.065 | 87.268 | 15.541 | 1.00 | 74.70 | 6 |
| ATOM | 8383 | CE2 | PHE | C | 637 | 32.128 | 86.221 | 14.963 | 1.00 | 75.81 | 6 |
| ATOM | 8384 | CZ | PHE | C | 637 | 30.974 | 86.880 | 14.586 | 1.00 | 74.99 | 6 |
| ATOM | 8385 | C | PHE | C | 637 | 30.256 | 84.026 | 17.964 | 1.00 | 20.74 | 6 |
| ATOM | 8386 | O | PHE | C | 637 | 30.760 | 83.636 | 16.902 | 1.00 | 19.02 | 8 |
| ATOM | 8387 | N | ALA | C | 638 | 28.944 | 83.994 | 18.223 | 1.00 | 75.28 | 7 |
| ATOM | 8388 | CA | ALA | C | 638 | 27.937 | 83.501 | 17.267 | 1.00 | 77.77 | 6 |
| ATOM | 8389 | CB | ALA | C | 638 | 27.460 | 82.101 | 17.667 | 1.00 | 78.00 | 6 |
| ATOM | 8390 | C | ALA | C | 638 | 26.736 | 84.452 | 17.159 | 1.00 | 78.88 | 6 |
| ATOM | 8391 | O | ALA | C | 638 | 26.675 | 85.460 | 17.868 | 1.00 | 78.96 | 8 |
| ATOM | 8392 | N | GLN | C | 1639 | 25.776 | 84.124 | 16.291 | 1.00 | 42.30 | 7 |
| ATOM | 8393 | CA | GLN | C | 1639 | 24.609 | 84.988 | 16.082 | 1.00 | 44.59 | 6 |
| ATOM | 8394 | CB | GLN | C | 1639 | 24.843 | 85.849 | 14.841 | 1.00 | 96.59 | 6 |
| ATOM | 8395 | CG | GLN | C | 1639 | 25.848 | 86.957 | 15.050 | 1.00 | 99.50 | 6 |
| ATOM | 8396 | CD | GLN | C | 1639 | 26.278 | 87.593 | 13.753 | 1.00 | 102.01 | 6 |
| ATOM | 8397 | OE1 | GLN | C | 1639 | 25.450 | 87.942 | 12.920 | 1.00 | 105.55 | 8 |
| ATOM | 8398 | NE2 | GLN | C | 1639 | 27.582 | 87.757 | 13.576 | 1.00 | 102.38 | 7 |
| ATOM | 8399 | C | GLN | C | 1639 | 23.229 | 84.326 | 15.969 | 1.00 | 45.30 | 6 |
| ATOM | 8400 | O | GLN | C | 1639 | 22.943 | 83.634 | 15.003 | 1.00 | 44.35 | 8 |
| ATOM | 8401 | N | ARG | C | 640 | 22.373 | 84.580 | 16.956 | 1.00 | 59.63 | 7 |
| ATOM | 8402 | CA | ARG | C | 640 | 21.009 | 84.051 | 16.990 | 1.00 | 61.89 | 6 |
| ATOM | 8403 | CB | ARG | C | 640 | 20.521 | 83.936 | 18.437 | 1.00 | 153.83 | 6 |
| ATOM | 8404 | CG | ARG | C | 640 | 19.029 | 83.679 | 18.568 | 1.00 | 156.43 | 6 |
| ATOM | 8405 | CD | ARG | C | 640 | 18.627 | 83.400 | 20.003 | 1.00 | 158.29 | 6 |
| ATOM | 8406 | NE | ARG | C | 640 | 17.216 | 83.041 | 20.101 | 1.00 | 160.47 | 7 |
| ATOM | 8407 | CZ | ARG | C | 640 | 16.638 | 82.066 | 19.403 | 1.00 | 161.22 | 6 |
| ATOM | 8408 | NH1 | ARG | C | 640 | 17.347 | 81.342 | 18.545 | 1.00 | 161.40 | 7 |
| ATOM | 8409 | NH2 | ARG | C | 640 | 15.345 | 81.811 | 19.563 | 1.00 | 161.77 | 7 |
| ATOM | 8410 | C | ARG | C | 640 | 20.085 | 84.987 | 16.216 | 1.00 | 63.50 | 6 |
| ATOM | 8411 | O | ARG | C | 640 | 20.331 | 86.186 | 16.144 | 1.00 | 63.90 | 8 |
| ATOM | 8412 | N | PRO | C | 641 | 19.006 | 84.450 | 15.629 | 1.00 | 62.03 | 7 |
| ATOM | 8413 | CD | PRO | C | 641 | 18.787 | 83.012 | 15.379 | 1.00 | 46.62 | 6 |
| ATOM | 8414 | CA | PRO | C | 641 | 18.054 | 85.250 | 14.855 | 1.00 | 63.40 | 6 |
| ATOM | 8415 | CB | PRO | C | 641 | 17.718 | 84.318 | 13.716 | 1.00 | 47.20 | 6 |
| ATOM | 8416 | CG | PRO | C | 641 | 17.567 | 83.015 | 14.462 | 1.00 | 46.99 | 6 |
| ATOM | 8417 | C | PRO | C | 641 | 16.802 | 85.628 | 15.643 | 1.00 | 65.41 | 6 |
| ATOM | 8418 | O | PRO | C | 641 | 16.645 | 85.220 | 16.797 | 1.00 | 65.47 | 8 |
| ATOM | 8419 | N | ARG | C | 642 | 15.913 | 86.392 | 15.002 | 1.00 | 70.26 | 7 |
| ATOM | 8420 | CA | ARG | C | 642 | 14.645 | 86.816 | 15.610 | 1.00 | 72.74 | 6 |
| ATOM | 8421 | CB | ARG | C | 642 | 14.877 | 87.954 | 16.618 | 1.00 | 116.38 | 6 |
| ATOM | 8422 | CG | ARG | C | 642 | 13.580 | 88.464 | 17.257 | 1.00 | 117.46 | 6 |
| ATOM | 8423 | CD | ARG | C | 642 | 13.809 | 89.516 | 18.344 | 1.00 | 118.32 | 6 |
| ATOM | 8424 | NE | ARG | C | 642 | 12.544 | 89.986 | 18.920 | 1.00 | 118.81 | 7 |
| ATOM | 8425 | CZ | ARG | C | 642 | 12.436 | 90.914 | 19.873 | 1.00 | 118.86 | 6 |
| ATOM | 8426 | NH1 | ARG | C | 642 | 13.519 | 91.493 | 20.382 | 1.00 | 117.93 | 7 |
| ATOM | 8427 | NH2 | ARG | C | 642 | 11.235 | 91.271 | 20.315 | 1.00 | 118.51 | 7 |
| ATOM | 8428 | C | ARG | C | 642 | 13.540 | 87.254 | 14.632 | 1.00 | 73.67 | 6 |
| ATOM | 8429 | O | ARG | C | 642 | 13.766 | 88.075 | 13.738 | 1.00 | 73.21 | 8 |
| ATOM | 8430 | N | VAL | C | 643 | 12.351 | 86.680 | 14.819 | 1.00 | 54.77 | 7 |
| ATOM | 8431 | CA | VAL | C | 643 | 11.147 | 87.009 | 14.044 | 1.00 | 57.83 | 6 |
| ATOM | 8432 | CB | VAL | C | 643 | 11.115 | 88.525 | 13.688 | 1.00 | 183.07 | 6 |
| ATOM | 8433 | CG1 | VAL | C | 643 | 9.861 | 88.856 | 12.896 | 1.00 | 184.54 | 6 |
| ATOM | 8434 | CG2 | VAL | C | 643 | 11.170 | 89.362 | 14.958 | 1.00 | 183.92 | 6 |
| ATOM | 8435 | C | VAL | C | 643 | 10.852 | 86.219 | 12.774 | 1.00 | 58.95 | 6 |
| ATOM | 8436 | O | VAL | C | 643 | 11.675 | 86.159 | 11.871 | 1.00 | 59.02 | 8 |
| ATOM | 8437 | N | ARG | C | 644 | 9.652 | 85.649 | 12.709 | 1.00 | 69.80 | 7 |
| ATOM | 8438 | CA | ARG | C | 644 | 9.210 | 84.865 | 11.557 | 1.00 | 72.46 | 6 |
| ATOM | 8439 | CB | ARG | C | 644 | 8.083 | 83.917 | 11.952 | 1.00 | 202.67 | 6 |
| ATOM | 8440 | CG | ARG | C | 644 | 8.467 | 82.802 | 12.888 | 1.00 | 204.91 | 6 |
| ATOM | 8441 | CD | ARG | C | 644 | 7.279 | 81.877 | 13.050 | 1.00 | 206.63 | 6 |
| ATOM | 8442 | NE | ARG | C | 644 | 7.601 | 80.669 | 13.796 | 1.00 | 208.87 | 7 |
| ATOM | 8443 | CZ | ARG | C | 644 | 6.779 | 79.632 | 13.909 | 1.00 | 208.87 | 6 |
| ATOM | 8444 | NH1 | ARG | C | 644 | 5.593 | 79.668 | 13.322 | 1.00 | 208.87 | 7 |
| ATOM | 8445 | NH2 | ARG | C | 644 | 7.139 | 78.561 | 14.598 | 1.00 | 208.87 | 7 |
| ATOM | 8446 | C | ARG | C | 644 | 8.692 | 85.742 | 10.422 | 1.00 | 73.10 | 6 |
| ATOM | 8447 | O | ARG | C | 644 | 9.176 | 85.667 | 9.287 | 1.00 | 72.87 | 8 |
| ATOM | 8448 | N | VAL | C | 645 | 7.683 | 86.552 | 10.734 | 1.00 | 127.85 | 7 |
| ATOM | 8449 | CA | VAL | C | 645 | 7.063 | 87.444 | 9.759 | 1.00 | 128.53 | 6 |
| ATOM | 8450 | CB | VAL | C | 645 | 5.737 | 86.859 | 9.258 | 1.00 | 109.79 | 6 |
| ATOM | 8451 | CG1 | VAL | C | 645 | 5.129 | 87.777 | 8.208 | 1.00 | 109.80 | 6 |
| ATOM | 8452 | CG2 | VAL | C | 645 | 5.966 | 85.463 | 8.703 | 1.00 | 109.41 | 6 |
| ATOM | 8453 | C | VAL | C | 645 | 6.779 | 88.813 | 10.365 | 1.00 | 128.82 | 6 |
| ATOM | 8454 | O | VAL | C | 645 | 5.784 | 88.988 | 11.072 | 1.00 | 129.18 | 8 |
| ATOM | 8455 | N | GLY | C | 646 | 7.645 | 89.782 | 10.078 | 1.00 | 94.58 | 7 |
| ATOM | 8456 | CA | GLY | C | 646 | 7.461 | 91.115 | 10.630 | 1.00 | 94.93 | 6 |
| ATOM | 8457 | C | GLY | C | 646 | 7.932 | 92.247 | 9.738 | 1.00 | 95.18 | 6 |
| ATOM | 8458 | O | GLY | C | 646 | 8.204 | 92.038 | 8.553 | 1.00 | 95.22 | 8 |
| ATOM | 8459 | N | ALA | C | 647 | 8.040 | 93.445 | 10.311 | 1.00 | 89.09 | 7 |
| ATOM | 8460 | CA | ALA | C | 647 | 8.461 | 94.615 | 9.554 | 1.00 | 88.98 | 6 |
| ATOM | 8461 | CB | ALA | C | 647 | 7.434 | 95.716 | 9.711 | 1.00 | 122.12 | 6 |
| ATOM | 8462 | C | ALA | C | 647 | 9.851 | 95.141 | 9.906 | 1.00 | 89.28 | 6 |
| ATOM | 8463 | O | ALA | C | 647 | 10.805 | 94.377 | 10.027 | 1.00 | 89.89 | 8 |
| ATOM | 8464 | N | ARG | C | 648 | 9.969 | 96.453 | 10.066 | 1.00 | 89.53 | 7 |
| ATOM | 8465 | CA | ARG | C | 648 | 11.267 | 97.029 | 10.359 | 1.00 | 90.09 | 6 |
| ATOM | 8466 | CB | ARG | C | 648 | 11.365 | 98.462 | 9.853 | 1.00 | 95.01 | 6 |
| ATOM | 8467 | CG | ARG | C | 648 | 12.733 | 99.008 | 10.169 | 1.00 | 95.96 | 6 |
| ATOM | 8468 | CD | ARG | C | 648 | 13.113 | 100.253 | 9.425 | 1.00 | 95.74 | 6 |
| ATOM | 8469 | NE | ARG | C | 648 | 14.569 | 100.324 | 9.347 | 1.00 | 96.24 | 7 |
| ATOM | 8470 | CZ | ARG | C | 648 | 15.381 | 100.194 | 10.391 | 1.00 | 97.70 | 6 |
| ATOM | 8471 | NH1 | ARG | C | 648 | 14.885 | 99.992 | 11.603 | 1.00 | 98.66 | 7 |
| ATOM | 8472 | NH2 | ARG | C | 648 | 16.692 | 100.245 | 10.222 | 1.00 | 98.84 | 7 |
| ATOM | 8473 | C | ARG | C | 648 | 11.747 | 97.013 | 11.798 | 1.00 | 90.53 | 6 |
| ATOM | 8474 | O | ARG | C | 648 | 12.850 | 96.549 | 12.076 | 1.00 | 90.84 | 8 |
| ATOM | 8475 | N | ALA | C | 649 | 10.950 | 97.535 | 12.720 | 1.00 | 185.27 | 7 |
| ATOM | 8476 | CA | ALA | C | 649 | 11.401 | 97.552 | 14.099 | 1.00 | 185.25 | 6 |
| ATOM | 8477 | CB | ALA | C | 649 | 12.353 | 98.725 | 14.309 | 1.00 | 92.17 | 6 |
| ATOM | 8478 | C | ALA | C | 649 | 10.292 | 97.598 | 15.132 | 1.00 | 185.02 | 6 |
| ATOM | 8479 | O | ALA | C | 649 | 9.108 | 97.674 | 14.805 | 1.00 | 185.90 | 8 |
| ATOM | 8480 | N | ALA | C | 650 | 10.714 | 97.542 | 16.389 | 1.00 | 156.11 | 7 |
| ATOM | 8481 | CA | ALA | C | 650 | 9.834 | 97.581 | 17.549 | 1.00 | 155.18 | 6 |
| ATOM | 8482 | CB | ALA | C | 650 | 9.242 | 96.201 | 17.806 | 1.00 | 159.77 | 6 |
| ATOM | 8483 | C | ALA | C | 650 | 10.744 | 97.991 | 18.699 | 1.00 | 154.07 | 6 |
| ATOM | 8484 | O | ALA | C | 650 | 11.183 | 97.151 | 19.484 | 1.00 | 153.68 | 8 |
| ATOM | 8485 | N | ALA | C | 651 | 11.034 | 99.285 | 18.784 | 1.00 | 198.58 | 7 |
| ATOM | 8486 | CA | ALA | C | 651 | 11.928 | 99.803 | 19.812 | 1.00 | 197.30 | 6 |
| ATOM | 8487 | CB | ALA | C | 651 | 11.470 | 99.352 | 21.193 | 1.00 | 113.54 | 6 |
| ATOM | 8488 | C | ALA | C | 651 | 13.319 | 99.255 | 19.501 | 1.00 | 195.92 | 6 |
| ATOM | 8489 | O | ALA | C | 651 | 14.216 | 99.263 | 20.346 | 1.00 | 197.13 | 8 |
| ATOM | 8490 | N | GLY | C | 652 | 13.476 | 98.778 | 18.269 | 1.00 | 157.04 | 7 |
| ATOM | 8491 | CA | GLY | C | 652 | 14.741 | 98.227 | 17.818 | 1.00 | 152.46 | 6 |
| ATOM | 8492 | C | GLY | C | 652 | 14.562 | 97.304 | 16.623 | 1.00 | 149.73 | 6 |
| ATOM | 8493 | O | GLY | C | 652 | 13.796 | 96.341 | 16.693 | 1.00 | 150.26 | 8 |
| ATOM | 8494 | N | ASP | C | 653 | 15.254 | 97.600 | 15.523 | 1.00 | 75.23 | 7 |
| ATOM | 8495 | CA | ASP | C | 653 | 15.172 | 96.778 | 14.315 | 1.00 | 70.01 | 6 |
| ATOM | 8496 | CB | ASP | C | 653 | 15.286 | 97.659 | 13.068 | 1.00 | 49.97 | 6 |
| ATOM | 8497 | CG | ASP | C | 653 | 16.723 | 97.983 | 12.711 | 1.00 | 49.50 | 6 |
| ATOM | 8498 | OD1 | ASP | C | 653 | 17.467 | 98.489 | 13.582 | 1.00 | 49.55 | 8 |
| ATOM | 8499 | OD2 | ASP | C | 653 | 17.109 | 97.725 | 11.551 | 1.00 | 49.99 | 8 |
| ATOM | 8500 | C | ASP | C | 653 | 16.320 | 95.763 | 14.359 | 1.00 | 66.17 | 6 |
| ATOM | 8501 | O | ASP | C | 653 | 17.014 | 95.529 | 13.366 | 1.00 | 64.12 | 8 |
| ATOM | 8502 | N | LEU | C | 654 | 16.512 | 95.170 | 15.533 | 1.00 | 78.22 | 7 |
| ATOM | 8503 | CA | LEU | C | 654 | 17.566 | 94.195 | 15.721 | 1.00 | 75.71 | 6 |
| ATOM | 8504 | CB | LEU | C | 654 | 17.319 | 93.346 | 16.968 | 1.00 | 44.26 | 6 |
| ATOM | 8505 | CG | LEU | C | 654 | 18.210 | 92.105 | 17.131 | 1.00 | 43.81 | 6 |
| ATOM | 8506 | CD1 | LEU | C | 654 | 19.657 | 92.490 | 16.975 | 1.00 | 43.96 | 6 |
| ATOM | 8507 | CD2 | LEU | C | 654 | 17.978 | 91.472 | 18.485 | 1.00 | 43.72 | 6 |
| ATOM | 8508 | C | LEU | C | 654 | 17.693 | 93.285 | 14.524 | 1.00 | 73.51 | 6 |
| ATOM | 8509 | O | LEU | C | 654 | 18.497 | 93.545 | 13.630 | 1.00 | 74.58 | 8 |
| ATOM | 8510 | N | LEU | C | 655 | 16.890 | 92.229 | 14.494 | 1.00 | 56.02 | 7 |
| ATOM | 8511 | CA | LEU | C | 655 | 16.978 | 91.285 | 13.399 | 1.00 | 52.20 | 6 |
| ATOM | 8512 | CB | LEU | C | 655 | 17.115 | 92.045 | 12.060 | 1.00 | 25.83 | 6 |
| ATOM | 8513 | CG | LEU | C | 655 | 17.371 | 91.406 | 10.685 | 1.00 | 22.71 | 6 |
| ATOM | 8514 | CD1 | LEU | C | 655 | 16.407 | 91.979 | 9.681 | 1.00 | 22.40 | 6 |
| ATOM | 8515 | CD2 | LEU | C | 655 | 18.773 | 91.694 | 10.207 | 1.00 | 19.92 | 6 |
| ATOM | 8516 | C | LEU | C | 655 | 18.233 | 90.473 | 13.713 | 1.00 | 51.40 | 6 |
| ATOM | 8517 | O | LEU | C | 655 | 19.317 | 90.811 | 13.256 | 1.00 | 52.89 | 8 |
| ATOM | 8518 | N | ALA | C | 656 | 18.084 | 89.435 | 14.538 | 1.00 | 38.13 | 7 |
| ATOM | 8519 | CA | ALA | C | 656 | 19.194 | 88.547 | 14.917 | 1.00 | 36.10 | 6 |
| ATOM | 8520 | CB | ALA | C | 656 | 19.857 | 87.985 | 13.654 | 1.00 | 122.83 | 6 |
| ATOM | 8521 | C | ALA | C | 656 | 20.266 | 89.151 | 15.842 | 1.00 | 34.39 | 6 |
| ATOM | 8522 | O | ALA | C | 656 | 20.908 | 90.151 | 15.507 | 1.00 | 33.57 | 8 |
| ATOM | 8523 | N | ASP | C | 657 | 20.466 | 88.518 | 16.998 | 1.00 | 38.96 | 7 |
| ATOM | 8524 | CA | ASP | C | 657 | 21.441 | 88.981 | 17.980 | 1.00 | 37.30 | 6 |
| ATOM | 8525 | CB | ASP | C | 657 | 21.193 | 88.353 | 19.355 | 1.00 | 27.97 | 6 |
| ATOM | 8526 | CG | ASP | C | 657 | 20.259 | 89.188 | 20.226 | 1.00 | 27.28 | 6 |
| ATOM | 8527 | OD1 | ASP | C | 657 | 20.411 | 90.432 | 20.260 | 1.00 | 24.24 | 8 |
| ATOM | 8528 | OD2 | ASP | C | 657 | 19.379 | 88.596 | 20.889 | 1.00 | 27.26 | 8 |
| ATOM | 8529 | C | ASP | C | 657 | 22.881 | 88.732 | 17.602 | 1.00 | 37.25 | 6 |
| ATOM | 8530 | O | ASP | C | 657 | 23.247 | 88.787 | 16.439 | 1.00 | 37.78 | 8 |
| ATOM | 8531 | N | GLY | C | 658 | 23.701 | 88.475 | 18.608 | 1.00 | 64.11 | 7 |
| ATOM | 8532 | CA | GLY | C | 658 | 25.104 | 88.240 | 18.365 | 1.00 | 65.39 | 6 |
| ATOM | 8533 | C | GLY | C | 658 | 25.714 | 87.456 | 19.502 | 1.00 | 65.46 | 6 |
| ATOM | 8534 | O | GLY | C | 658 | 25.029 | 86.666 | 20.150 | 1.00 | 65.70 | 8 |
| ATOM | 8535 | N | PRO | C | 659 | 27.007 | 87.663 | 19.781 | 1.00 | 30.43 | 7 |
| ATOM | 8536 | CD | PRO | C | 659 | 27.972 | 88.389 | 18.941 | 1.00 | 54.09 | 6 |
| ATOM | 8537 | CA | PRO | C | 659 | 27.702 | 86.958 | 20.853 | 1.00 | 30.48 | 6 |
| ATOM | 8538 | CB | PRO | C | 659 | 29.132 | 86.917 | 20.335 | 1.00 | 52.79 | 6 |
| ATOM | 8539 | CG | PRO | C | 659 | 29.266 | 88.245 | 19.723 | 1.00 | 54.10 | 6 |
| ATOM | 8540 | C | PRO | C | 659 | 27.607 | 87.532 | 22.276 | 1.00 | 31.00 | 6 |
| ATOM | 8541 | O | PRO | C | 659 | 28.504 | 87.335 | 23.090 | 1.00 | 29.62 | 8 |
| ATOM | 8542 | N | ALA | C | 660 | 26.536 | 88.230 | 22.602 | 1.00 | 77.18 | 7 |
| ATOM | 8543 | CA | ALA | C | 660 | 26.448 | 88.742 | 23.954 | 1.00 | 78.37 | 6 |
| ATOM | 8544 | CB | ALA | C | 660 | 27.750 | 89.379 | 24.366 | 1.00 | 13.87 | 6 |
| ATOM | 8545 | C | ALA | C | 660 | 25.331 | 89.730 | 24.102 | 1.00 | 78.71 | 6 |
| ATOM | 8546 | O | ALA | C | 660 | 25.219 | 90.387 | 25.126 | 1.00 | 79.59 | 8 |
| ATOM | 8547 | N | SER | C | 661 | 24.504 | 89.849 | 23.079 | 1.00 | 42.03 | 7 |
| ATOM | 8548 | CA | SER | C | 661 | 23.391 | 90.763 | 23.172 | 1.00 | 42.57 | 6 |
| ATOM | 8549 | CB | SER | C | 661 | 23.083 | 91.354 | 21.808 | 1.00 | 54.64 | 6 |
| ATOM | 8550 | OG | SER | C | 661 | 22.727 | 90.321 | 20.919 | 1.00 | 57.11 | 8 |
| ATOM | 8551 | C | SER | C | 661 | 22.188 | 89.979 | 23.675 | 1.00 | 41.98 | 6 |
| ATOM | 8552 | O | SER | C | 661 | 21.934 | 88.861 | 23.231 | 1.00 | 41.17 | 8 |
| ATOM | 8553 | N | GLU | C | 662 | 21.477 | 90.557 | 24.633 | 1.00 | 66.98 | 7 |
| ATOM | 8554 | CA | GLU | C | 662 | 20.268 | 89.950 | 25.182 | 1.00 | 66.51 | 6 |
| ATOM | 8555 | CB | GLU | C | 662 | 20.123 | 90.309 | 26.672 | 1.00 | 53.91 | 6 |
| ATOM | 8556 | CG | GLU | C | 662 | 18.766 | 90.005 | 27.356 | 1.00 | 53.80 | 6 |
| ATOM | 8557 | CD | GLU | C | 662 | 18.369 | 88.542 | 27.318 | 1.00 | 53.96 | 6 |
| ATOM | 8558 | OE1 | GLU | C | 662 | 17.677 | 88.087 | 28.258 | 1.00 | 54.83 | 8 |
| ATOM | 8559 | OE2 | GLU | C | 662 | 18.733 | 87.852 | 26.343 | 1.00 | 53.54 | 8 |
| ATOM | 8560 | C | GLU | C | 662 | 19.181 | 90.611 | 24.355 | 1.00 | 65.72 | 6 |
| ATOM | 8561 | O | GLU | C | 662 | 18.189 | 91.112 | 24.875 | 1.00 | 65.71 | 8 |
| ATOM | 8562 | N | GLU | C | 663 | 19.409 | 90.616 | 23.051 | 1.00 | 39.28 | 7 |
| ATOM | 8563 | CA | GLU | C | 663 | 18.510 | 91.226 | 22.100 | 1.00 | 38.54 | 6 |
| ATOM | 8564 | CB | GLU | C | 663 | 17.065 | 91.104 | 22.557 | 1.00 | 54.30 | 6 |
| ATOM | 8565 | CG | GLU | C | 663 | 16.650 | 89.694 | 22.870 | 1.00 | 55.06 | 6 |
| ATOM | 8566 | CD | GLU | C | 663 | 15.207 | 89.626 | 23.282 | 1.00 | 54.67 | 6 |
| ATOM | 8567 | OE1 | GLU | C | 663 | 14.359 | 90.093 | 22.495 | 1.00 | 55.92 | 8 |
| ATOM | 8568 | OE2 | GLU | C | 663 | 14.920 | 89.118 | 24.387 | 1.00 | 53.70 | 8 |
| ATOM | 8569 | C | GLU | C | 663 | 18.891 | 92.686 | 21.983 | 1.00 | 38.12 | 6 |
| ATOM | 8570 | O | GLU | C | 663 | 18.158 | 93.564 | 22.431 | 1.00 | 38.14 | 8 |
| ATOM | 8571 | N | GLY | C | 664 | 20.051 | 92.939 | 21.385 | 1.00 | 75.46 | 7 |
| ATOM | 8572 | CA | GLY | C | 664 | 20.519 | 94.304 | 21.209 | 1.00 | 74.14 | 6 |
| ATOM | 8573 | C | GLY | C | 664 | 21.054 | 94.929 | 22.487 | 1.00 | 73.09 | 6 |
| ATOM | 8574 | O | GLY | C | 664 | 21.554 | 96.057 | 22.474 | 1.00 | 73.85 | 8 |
| ATOM | 8575 | N | PHE | C | 665 | 20.950 | 94.195 | 23.591 | 1.00 | 37.20 | 7 |
| ATOM | 8576 | CA | PHE | C | 665 | 21.421 | 94.676 | 24.883 | 1.00 | 34.13 | 6 |
| ATOM | 8577 | CB | PHE | C | 665 | 20.357 | 94.427 | 25.940 | 1.00 | 42.35 | 6 |
| ATOM | 8578 | CG | PHE | C | 665 | 19.212 | 95.380 | 25.881 | 1.00 | 43.12 | 6 |
| ATOM | 8579 | CD1 | PHE | C | 665 | 17.948 | 94.980 | 26.299 | 1.00 | 44.68 | 6 |
| ATOM | 8580 | CD2 | PHE | C | 665 | 19.400 | 96.688 | 25.427 | 1.00 | 42.37 | 6 |
| ATOM | 8581 | CE1 | PHE | C | 665 | 16.882 | 95.867 | 26.266 | 1.00 | 45.21 | 6 |
| ATOM | 8582 | CE2 | PHE | C | 665 | 18.352 | 97.585 | 25.388 | 1.00 | 41.86 | 6 |
| ATOM | 8583 | CZ | PHE | C | 665 | 17.085 | 97.184 | 25.805 | 1.00 | 44.49 | 6 |
| ATOM | 8584 | C | PHE | C | 665 | 22.685 | 93.970 | 25.301 | 1.00 | 31.94 | 6 |
| ATOM | 8585 | O | PHE | C | 665 | 22.614 | 92.854 | 25.796 | 1.00 | 31.91 | 8 |
| ATOM | 8586 | N | LEU | C | 666 | 23.837 | 94.604 | 25.108 | 1.00 | 26.18 | 7 |
| ATOM | 8587 | CA | LEU | C | 666 | 25.097 | 93.970 | 25.491 | 1.00 | 25.71 | 6 |
| ATOM | 8588 | CB | LEU | C | 666 | 26.209 | 94.998 | 25.727 | 1.00 | 31.13 | 6 |
| ATOM | 8589 | CG | LEU | C | 666 | 27.517 | 94.552 | 26.401 | 1.00 | 29.22 | 6 |
| ATOM | 8590 | CD1 | LEU | C | 666 | 28.022 | 93.288 | 25.774 | 1.00 | 29.99 | 6 |
| ATOM | 8591 | CD2 | LEU | C | 666 | 28.543 | 95.671 | 26.276 | 1.00 | 27.86 | 6 |
| ATOM | 8592 | C | LEU | C | 666 | 24.825 | 93.259 | 26.781 | 1.00 | 24.91 | 6 |
| ATOM | 8593 | O | LEU | C | 666 | 24.372 | 93.879 | 27.742 | 1.00 | 23.84 | 8 |
| ATOM | 8594 | N | ALA | C | 667 | 25.085 | 91.955 | 26.775 | 1.00 | 60.95 | 7 |
| ATOM | 8595 | CA | ALA | C | 667 | 24.881 | 91.069 | 27.916 | 1.00 | 60.19 | 6 |
| ATOM | 8596 | CB | ALA | C | 667 | 23.666 | 90.197 | 27.670 | 1.00 | 13.87 | 6 |
| ATOM | 8597 | C | ALA | C | 667 | 26.131 | 90.212 | 28.047 | 1.00 | 59.25 | 6 |
| ATOM | 8598 | O | ALA | C | 667 | 26.208 | 89.109 | 27.512 | 1.00 | 59.69 | 8 |
| ATOM | 8599 | N | LEU | C | 668 | 27.114 | 90.739 | 28.758 | 1.00 | 28.60 | 7 |
| ATOM | 8600 | CA | LEU | C | 668 | 28.371 | 90.056 | 28.944 | 1.00 | 25.65 | 6 |
| ATOM | 8601 | CB | LEU | C | 668 | 29.358 | 91.001 | 29.571 | 1.00 | 13.87 | 6 |
| ATOM | 8602 | CG | LEU | C | 668 | 29.496 | 92.156 | 28.596 | 1.00 | 13.87 | 6 |
| ATOM | 8603 | CD1 | LEU | C | 668 | 30.270 | 93.297 | 29.228 | 1.00 | 13.87 | 6 |
| ATOM | 8604 | CD2 | LEU | C | 668 | 30.164 | 91.636 | 27.337 | 1.00 | 13.87 | 6 |
| ATOM | 8605 | C | LEU | C | 668 | 28.238 | 88.824 | 29.780 | 1.00 | 26.32 | 6 |
| ATOM | 8606 | O | LEU | C | 668 | 28.695 | 87.762 | 29.392 | 1.00 | 26.84 | 8 |
| ATOM | 8607 | N | GLY | C | 669 | 27.614 | 88.951 | 30.937 | 1.00 | 22.74 | 7 |
| ATOM | 8608 | CA | GLY | C | 669 | 27.442 | 87.785 | 31.780 | 1.00 | 23.73 | 6 |
| ATOM | 8609 | C | GLY | C | 669 | 25.982 | 87.575 | 32.103 | 1.00 | 23.58 | 6 |
| ATOM | 8610 | O | GLY | C | 669 | 25.115 | 88.218 | 31.525 | 1.00 | 21.58 | 8 |
| ATOM | 8611 | N | GLN | C | 670 | 25.703 | 86.669 | 33.024 | 1.00 | 20.32 | 7 |
| ATOM | 8612 | CA | GLN | C | 670 | 24.336 | 86.407 | 33.418 | 1.00 | 22.32 | 6 |
| ATOM | 8613 | CB | GLN | C | 670 | 24.067 | 84.927 | 33.456 | 1.00 | 46.85 | 6 |
| ATOM | 8614 | CG | GLN | C | 670 | 24.178 | 84.247 | 32.149 | 1.00 | 49.88 | 6 |
| ATOM | 8615 | CD | GLN | C | 670 | 23.795 | 82.812 | 32.291 | 1.00 | 51.68 | 6 |
| ATOM | 8616 | OE1 | GLN | C | 670 | 24.560 | 82.000 | 32.824 | 1.00 | 52.07 | 8 |
| ATOM | 8617 | NE2 | GLN | C | 670 | 22.587 | 82.483 | 31.850 | 1.00 | 53.08 | 7 |
| ATOM | 8618 | C | GLN | C | 670 | 24.157 | 86.928 | 34.810 | 1.00 | 22.85 | 6 |
| ATOM | 8619 | O | GLN | C | 670 | 25.033 | 86.728 | 35.646 | 1.00 | 23.26 | 8 |
| ATOM | 8620 | N | ASN | C | 671 | 23.028 | 87.587 | 35.062 | 1.00 | 32.84 | 7 |
| ATOM | 8621 | CA | ASN | C | 671 | 22.739 | 88.117 | 36.394 | 1.00 | 33.83 | 6 |
| ATOM | 8622 | CB | ASN | C | 671 | 21.971 | 89.454 | 36.329 | 1.00 | 36.11 | 6 |
| ATOM | 8623 | CG | ASN | C | 671 | 20.866 | 89.458 | 35.293 | 1.00 | 37.42 | 6 |
| ATOM | 8624 | OD1 | ASN | C | 671 | 20.053 | 88.538 | 35.225 | 1.00 | 38.52 | 8 |
| ATOM | 8625 | ND2 | ASN | C | 671 | 20.823 | 90.512 | 34.489 | 1.00 | 36.92 | 7 |
| ATOM | 8626 | C | ASN | C | 671 | 21.959 | 87.093 | 37.197 | 1.00 | 32.30 | 6 |
| ATOM | 8627 | O | ASN | C | 671 | 21.011 | 86.479 | 36.713 | 1.00 | 30.24 | 8 |
| ATOM | 8628 | N | VAL | C | 672 | 22.377 | 86.897 | 38.435 | 1.00 | 40.81 | 7 |
| ATOM | 8629 | CA | VAL | C | 672 | 21.708 | 85.924 | 39.253 | 1.00 | 40.63 | 6 |
| ATOM | 8630 | CB | VAL | C | 672 | 22.535 | 84.636 | 39.314 | 1.00 | 34.74 | 6 |
| ATOM | 8631 | CG1 | VAL | C | 672 | 23.565 | 84.694 | 40.436 | 1.00 | 34.96 | 6 |
| ATOM | 8632 | CG2 | VAL | C | 672 | 21.609 | 83.474 | 39.443 | 1.00 | 35.10 | 6 |
| ATOM | 8633 | C | VAL | C | 672 | 21.419 | 86.452 | 40.646 | 1.00 | 40.17 | 6 |
| ATOM | 8634 | O | VAL | C | 672 | 21.634 | 87.631 | 40.928 | 1.00 | 41.23 | 8 |
| ATOM | 8635 | N | LEU | C | 673 | 20.924 | 85.579 | 41.514 | 1.00 | 22.87 | 7 |
| ATOM | 8636 | CA | LEU | C | 673 | 20.576 | 85.979 | 42.868 | 1.00 | 22.05 | 6 |
| ATOM | 8637 | CB | LEU | C | 673 | 19.217 | 85.357 | 43.238 | 1.00 | 34.54 | 6 |
| ATOM | 8638 | CG | LEU | C | 673 | 18.323 | 85.858 | 44.377 | 1.00 | 32.29 | 6 |
| ATOM | 8639 | CD1 | LEU | C | 673 | 16.967 | 86.182 | 43.789 | 1.00 | 29.25 | 6 |
| ATOM | 8640 | CD2 | LEU | C | 673 | 18.189 | 84.800 | 45.481 | 1.00 | 32.17 | 6 |
| ATOM | 8641 | C | LEU | C | 673 | 21.660 | 85.572 | 43.865 | 1.00 | 21.61 | 6 |
| ATOM | 8642 | O | LEU | C | 673 | 22.020 | 84.399 | 43.978 | 1.00 | 21.08 | 8 |
| ATOM | 8643 | N | VAL | C | 674 | 22.180 | 86.568 | 44.572 | 1.00 | 13.87 | 7 |
| ATOM | 8644 | CA | VAL | C | 674 | 23.220 | 86.383 | 45.572 | 1.00 | 13.87 | 6 |
| ATOM | 8645 | CB | VAL | C | 674 | 24.458 | 87.262 | 45.262 | 1.00 | 30.66 | 6 |
| ATOM | 8646 | CG1 | VAL | C | 674 | 24.017 | 88.640 | 44.823 | 1.00 | 33.54 | 6 |
| ATOM | 8647 | CG2 | VAL | C | 674 | 25.342 | 87.393 | 46.491 | 1.00 | 30.32 | 6 |
| ATOM | 8648 | C | VAL | C | 674 | 22.686 | 86.782 | 46.925 | 1.00 | 13.87 | 6 |
| ATOM | 8649 | O | VAL | C | 674 | 21.602 | 87.325 | 47.043 | 1.00 | 13.87 | 8 |
| ATOM | 8650 | N | ALA | C | 675 | 23.455 | 86.498 | 47.954 | 1.00 | 21.92 | 7 |
| ATOM | 8651 | CA | ALA | C | 675 | 23.078 | 86.855 | 49.301 | 1.00 | 22.72 | 6 |
| ATOM | 8652 | CB | ALA | C | 675 | 22.341 | 85.711 | 49.971 | 1.00 | 135.95 | 6 |
| ATOM | 8653 | C | ALA | C | 675 | 24.433 | 87.040 | 49.915 | 1.00 | 22.35 | 6 |
| ATOM | 8654 | O | ALA | C | 675 | 25.244 | 86.122 | 49.885 | 1.00 | 23.65 | 8 |
| ATOM | 8655 | N | ILE | C | 676 | 24.699 | 88.233 | 50.433 | 1.00 | 18.20 | 7 |
| ATOM | 8656 | CA | ILE | C | 676 | 25.986 | 88.502 | 51.025 | 1.00 | 16.01 | 6 |
| ATOM | 8657 | CB | ILE | C | 676 | 26.191 | 89.988 | 51.264 | 1.00 | 13.87 | 6 |
| ATOM | 8658 | CG2 | ILE | C | 676 | 27.531 | 90.209 | 51.895 | 1.00 | 13.87 | 6 |
| ATOM | 8659 | CG1 | ILE | C | 676 | 26.176 | 90.749 | 49.937 | 1.00 | 13.87 | 6 |
| ATOM | 8660 | CD1 | ILE | C | 676 | 24.804 | 91.136 | 49.467 | 1.00 | 13.87 | 6 |
| ATOM | 8661 | C | ILE | C | 676 | 26.031 | 87.764 | 52.330 | 1.00 | 15.94 | 6 |
| ATOM | 8662 | O | ILE | C | 676 | 24.997 | 87.431 | 52.860 | 1.00 | 16.67 | 8 |
| ATOM | 8663 | N | MET | C | 677 | 27.232 | 87.475 | 52.812 | 1.00 | 23.08 | 7 |
| ATOM | 8664 | CA | MET | C | 677 | 27.442 | 86.777 | 54.073 | 1.00 | 23.84 | 6 |
| ATOM | 8665 | CB | MET | C | 677 | 26.183 | 86.042 | 54.536 | 1.00 | 21.85 | 6 |
| ATOM | 8666 | CG | MET | C | 677 | 25.858 | 84.812 | 53.737 | 1.00 | 20.37 | 6 |
| ATOM | 8667 | SD | MET | C | 677 | 24.471 | 83.914 | 54.420 | 1.00 | 21.77 | 16 |
| ATOM | 8668 | CE | MET | C | 677 | 25.130 | 83.561 | 56.108 | 1.00 | 20.08 | 6 |
| ATOM | 8669 | C | MET | C | 677 | 28.548 | 85.762 | 53.918 | 1.00 | 26.58 | 6 |
| ATOM | 8670 | O | MET | C | 677 | 28.632 | 85.079 | 52.905 | 1.00 | 28.10 | 8 |
| ATOM | 8671 | N | PRO | C | 678 | 29.425 | 85.650 | 54.918 | 1.00 | 51.11 | 7 |
| ATOM | 8672 | CD | PRO | C | 678 | 29.440 | 86.280 | 56.251 | 1.00 | 13.87 | 6 |
| ATOM | 8673 | CA | PRO | C | 678 | 30.496 | 84.658 | 54.772 | 1.00 | 53.91 | 6 |
| ATOM | 8674 | CB | PRO | C | 678 | 31.322 | 84.861 | 56.035 | 1.00 | 14.90 | 6 |
| ATOM | 8675 | CG | PRO | C | 678 | 30.264 | 85.309 | 57.055 | 1.00 | 13.87 | 6 |
| ATOM | 8676 | C | PRO | C | 678 | 29.846 | 83.287 | 54.740 | 1.00 | 56.46 | 6 |
| ATOM | 8677 | O | PRO | C | 678 | 28.678 | 83.171 | 55.113 | 1.00 | 59.26 | 8 |
| ATOM | 8678 | N | PHE | C | 679 | 30.565 | 82.260 | 54.288 | 1.00 | 30.38 | 7 |
| ATOM | 8679 | CA | PHE | C | 679 | 29.992 | 80.909 | 54.297 | 1.00 | 29.12 | 6 |
| ATOM | 8680 | CB | PHE | C | 679 | 29.492 | 80.494 | 52.920 | 1.00 | 41.93 | 6 |
| ATOM | 8681 | CG | PHE | C | 679 | 28.438 | 79.436 | 52.977 | 1.00 | 43.64 | 6 |
| ATOM | 8682 | CD1 | PHE | C | 679 | 27.099 | 79.782 | 53.094 | 1.00 | 44.45 | 6 |
| ATOM | 8683 | CD2 | PHE | C | 679 | 28.789 | 78.092 | 52.995 | 1.00 | 44.04 | 6 |
| ATOM | 8684 | CE1 | PHE | C | 679 | 26.130 | 78.807 | 53.233 | 1.00 | 44.58 | 6 |
| ATOM | 8685 | CE2 | PHE | C | 679 | 27.827 | 77.107 | 53.133 | 1.00 | 42.72 | 6 |
| ATOM | 8686 | CZ | PHE | C | 679 | 26.494 | 77.462 | 53.254 | 1.00 | 43.82 | 6 |
| ATOM | 8687 | C | PHE | C | 679 | 30.986 | 79.868 | 54.794 | 1.00 | 28.07 | 6 |
| ATOM | 8688 | O | PHE | C | 679 | 31.831 | 79.403 | 54.042 | 1.00 | 28.71 | 8 |
| ATOM | 8689 | N | ASP | C | 680 | 30.865 | 79.499 | 56.064 | 1.00 | 26.45 | 7 |
| ATOM | 8690 | CA | ASP | C | 680 | 31.767 | 78.537 | 56.698 | 1.00 | 27.76 | 6 |
| ATOM | 8691 | CB | ASP | C | 680 | 31.149 | 77.129 | 56.716 | 1.00 | 62.51 | 6 |
| ATOM | 8692 | CG | ASP | C | 680 | 30.244 | 76.894 | 57.930 | 1.00 | 66.52 | 6 |
| ATOM | 8693 | OD1 | ASP | C | 680 | 29.046 | 77.257 | 57.886 | 1.00 | 69.84 | 8 |
| ATOM | 8694 | OD2 | ASP | C | 680 | 30.738 | 76.349 | 58.940 | 1.00 | 67.51 | 8 |
| ATOM | 8695 | C | ASP | C | 680 | 33.158 | 78.489 | 56.073 | 1.00 | 26.14 | 6 |
| ATOM | 8696 | O | ASP | C | 680 | 33.789 | 77.442 | 56.040 | 1.00 | 25.67 | 8 |
| ATOM | 8697 | N | GLY | C | 681 | 33.630 | 79.626 | 55.577 | 1.00 | 47.60 | 7 |
| ATOM | 8698 | CA | GLY | C | 681 | 34.952 | 79.673 | 54.986 | 1.00 | 46.00 | 6 |
| ATOM | 8699 | C | GLY | C | 681 | 35.135 | 79.423 | 53.501 | 1.00 | 44.37 | 6 |
| ATOM | 8700 | O | GLY | C | 681 | 36.270 | 79.390 | 53.012 | 1.00 | 44.60 | 8 |
| ATOM | 8701 | N | TYR | C | 682 | 34.054 | 79.251 | 52.757 | 1.00 | 27.30 | 7 |
| ATOM | 8702 | CA | TYR | C | 682 | 34.228 | 79.007 | 51.341 | 1.00 | 24.29 | 6 |
| ATOM | 8703 | CB | TYR | C | 682 | 33.100 | 78.138 | 50.812 | 1.00 | 20.76 | 6 |
| ATOM | 8704 | CG | TYR | C | 682 | 33.253 | 76.754 | 51.382 | 1.00 | 18.89 | 6 |
| ATOM | 8705 | CD1 | TYR | C | 682 | 34.437 | 76.051 | 51.207 | 1.00 | 19.01 | 6 |
| ATOM | 8706 | CE1 | TYR | C | 682 | 34.647 | 74.844 | 51.820 | 1.00 | 18.22 | 6 |
| ATOM | 8707 | CD2 | TYR | C | 682 | 32.274 | 76.193 | 52.188 | 1.00 | 19.31 | 6 |
| ATOM | 8708 | CE2 | TYR | C | 682 | 32.478 | 74.975 | 52.804 | 1.00 | 18.87 | 6 |
| ATOM | 8709 | CZ | TYR | C | 682 | 33.669 | 74.306 | 52.614 | 1.00 | 18.22 | 6 |
| ATOM | 8710 | OH | TYR | C | 682 | 33.881 | 73.082 | 53.198 | 1.00 | 18.76 | 8 |
| ATOM | 8711 | C | TYR | C | 682 | 34.397 | 80.260 | 50.546 | 1.00 | 23.01 | 6 |
| ATOM | 8712 | O | TYR | C | 682 | 34.791 | 80.205 | 49.395 | 1.00 | 24.02 | 8 |
| ATOM | 8713 | N | ASN | C | 683 | 34.103 | 81.394 | 51.169 | 1.00 | 13.87 | 7 |
| ATOM | 8714 | CA | ASN | C | 683 | 34.280 | 82.686 | 50.528 | 1.00 | 13.87 | 6 |
| ATOM | 8715 | CB | ASN | C | 683 | 32.940 | 83.456 | 50.347 | 1.00 | 18.87 | 6 |
| ATOM | 8716 | CG | ASN | C | 683 | 31.927 | 83.243 | 51.489 | 1.00 | 18.87 | 6 |
| ATOM | 8717 | OD1 | ASN | C | 683 | 32.142 | 82.466 | 52.412 | 1.00 | 19.81 | 8 |
| ATOM | 8718 | ND2 | ASN | C | 683 | 30.805 | 83.949 | 51.405 | 1.00 | 18.87 | 7 |
| ATOM | 8719 | C | ASN | C | 683 | 35.278 | 83.448 | 51.385 | 1.00 | 14.52 | 6 |
| ATOM | 8720 | O | ASN | C | 683 | 34.929 | 84.346 | 52.139 | 1.00 | 16.41 | 8 |
| ATOM | 8721 | N | PHE | C | 684 | 36.535 | 83.036 | 51.282 | 1.00 | 13.87 | 7 |
| ATOM | 8722 | CA | PHE | C | 684 | 37.633 | 83.645 | 52.024 | 1.00 | 13.87 | 6 |
| ATOM | 8723 | CB | PHE | C | 684 | 38.592 | 82.577 | 52.510 | 1.00 | 19.24 | 6 |
| ATOM | 8724 | CG | PHE | C | 684 | 39.629 | 83.067 | 53.478 | 1.00 | 18.28 | 6 |
| ATOM | 8725 | CD1 | PHE | C | 684 | 39.368 | 83.077 | 54.846 | 1.00 | 17.56 | 6 |
| ATOM | 8726 | CD2 | PHE | C | 684 | 40.898 | 83.407 | 53.035 | 1.00 | 17.56 | 6 |
| ATOM | 8727 | CE1 | PHE | C | 684 | 40.355 | 83.402 | 55.755 | 1.00 | 17.56 | 6 |
| ATOM | 8728 | CE2 | PHE | C | 684 | 41.888 | 83.733 | 53.937 | 1.00 | 17.92 | 6 |
| ATOM | 8729 | CZ | PHE | C | 684 | 41.618 | 83.726 | 55.303 | 1.00 | 17.56 | 6 |
| ATOM | 8730 | C | PHE | C | 684 | 38.375 | 84.586 | 51.105 | 1.00 | 13.87 | 6 |
| ATOM | 8731 | O | PHE | C | 684 | 38.571 | 84.314 | 49.931 | 1.00 | 13.87 | 8 |
| ATOM | 8732 | N | GLU | C | 685 | 38.801 | 85.701 | 51.657 | 1.00 | 69.69 | 7 |
| ATOM | 8733 | CA | GLU | C | 685 | 39.490 | 86.678 | 50.855 | 1.00 | 72.85 | 6 |
| ATOM | 8734 | CB | GLU | C | 685 | 40.741 | 86.065 | 50.227 | 1.00 | 47.62 | 6 |
| ATOM | 8735 | CG | GLU | C | 685 | 41.888 | 85.969 | 51.222 | 1.00 | 48.90 | 6 |
| ATOM | 8736 | CD | GLU | C | 685 | 42.390 | 87.340 | 51.685 | 1.00 | 50.71 | 6 |
| ATOM | 8737 | OE1 | GLU | C | 685 | 41.562 | 88.250 | 51.920 | 1.00 | 50.93 | 8 |
| ATOM | 8738 | OE2 | GLU | C | 685 | 43.623 | 87.508 | 51.830 | 1.00 | 52.91 | 8 |
| ATOM | 8739 | C | GLU | C | 685 | 38.545 | 87.217 | 49.794 | 1.00 | 74.22 | 6 |
| ATOM | 8740 | O | GLU | C | 685 | 37.999 | 88.311 | 49.957 | 1.00 | 76.00 | 8 |
| ATOM | 8741 | N | ASP | C | 686 | 38.310 | 86.462 | 48.728 | 1.00 | 55.73 | 7 |
| ATOM | 8742 | CA | ASP | C | 686 | 37.428 | 86.974 | 47.681 | 1.00 | 56.10 | 6 |
| ATOM | 8743 | CB | ASP | C | 686 | 38.239 | 87.893 | 46.766 | 1.00 | 52.07 | 6 |
| ATOM | 8744 | CG | ASP | C | 686 | 39.725 | 87.933 | 47.142 | 1.00 | 52.04 | 6 |
| ATOM | 8745 | OD1 | ASP | C | 686 | 40.357 | 86.856 | 47.286 | 1.00 | 50.57 | 8 |
| ATOM | 8746 | OD2 | ASP | C | 686 | 40.257 | 89.052 | 47.293 | 1.00 | 51.95 | 8 |
| ATOM | 8747 | C | ASP | C | 686 | 36.725 | 85.893 | 46.858 | 1.00 | 56.69 | 6 |
| ATOM | 8748 | O | ASP | C | 686 | 35.956 | 86.199 | 45.932 | 1.00 | 57.76 | 8 |
| ATOM | 8749 | N | ALA | C | 687 | 36.989 | 84.635 | 47.211 | 1.00 | 81.74 | 7 |
| ATOM | 8750 | CA | ALA | C | 687 | 36.405 | 83.483 | 46.531 | 1.00 | 79.22 | 6 |
| ATOM | 8751 | CB | ALA | C | 687 | 37.021 | 82.221 | 47.071 | 1.00 | 13.87 | 6 |
| ATOM | 8752 | C | ALA | C | 687 | 34.884 | 83.443 | 46.680 | 1.00 | 77.60 | 6 |
| ATOM | 8753 | O | ALA | C | 687 | 34.343 | 83.847 | 47.709 | 1.00 | 77.71 | 8 |
| ATOM | 8754 | N | ILE | C | 688 | 34.202 | 82.951 | 45.648 | 1.00 | 32.24 | 7 |
| ATOM | 8755 | CA | ILE | C | 688 | 32.743 | 82.882 | 45.646 | 1.00 | 29.34 | 6 |
| ATOM | 8756 | CB | ILE | C | 688 | 32.141 | 83.381 | 44.325 | 1.00 | 20.69 | 6 |
| ATOM | 8757 | CG2 | ILE | C | 688 | 30.903 | 84.139 | 44.633 | 1.00 | 21.41 | 6 |
| ATOM | 8758 | CG1 | ILE | C | 688 | 33.135 | 84.247 | 43.528 | 1.00 | 20.26 | 6 |
| ATOM | 8759 | CD1 | ILE | C | 688 | 33.379 | 85.668 | 44.055 | 1.00 | 19.57 | 6 |
| ATOM | 8760 | C | ILE | C | 688 | 32.247 | 81.460 | 45.804 | 1.00 | 28.39 | 6 |
| ATOM | 8761 | O | ILE | C | 688 | 32.988 | 80.523 | 45.531 | 1.00 | 29.02 | 8 |
| ATOM | 8762 | N | VAL | C | 689 | 30.993 | 81.301 | 46.226 | 1.00 | 28.34 | 7 |
| ATOM | 8763 | CA | VAL | C | 689 | 30.396 | 79.972 | 46.380 | 1.00 | 26.56 | 6 |
| ATOM | 8764 | CB | VAL | C | 689 | 29.970 | 79.721 | 47.826 | 1.00 | 13.87 | 6 |
| ATOM | 8765 | CG1 | VAL | C | 689 | 29.369 | 78.330 | 47.957 | 1.00 | 13.87 | 6 |
| ATOM | 8766 | CG2 | VAL | C | 689 | 31.178 | 79.845 | 48.744 | 1.00 | 13.87 | 6 |
| ATOM | 8767 | C | VAL | C | 689 | 29.193 | 79.776 | 45.446 | 1.00 | 27.03 | 6 |
| ATOM | 8768 | O | VAL | C | 689 | 28.212 | 80.493 | 45.520 | 1.00 | 27.57 | 8 |
| ATOM | 8769 | N | ILE | C | 690 | 29.279 | 78.794 | 44.563 | 1.00 | 13.87 | 7 |
| ATOM | 8770 | CA | ILE | C | 690 | 28.229 | 78.541 | 43.595 | 1.00 | 13.87 | 6 |
| ATOM | 8771 | CB | ILE | C | 690 | 28.802 | 77.930 | 42.270 | 1.00 | 31.31 | 6 |
| ATOM | 8772 | CG2 | ILE | C | 690 | 27.739 | 77.912 | 41.192 | 1.00 | 33.95 | 6 |
| ATOM | 8773 | CG1 | ILE | C | 690 | 30.008 | 78.717 | 41.757 | 1.00 | 32.30 | 6 |
| ATOM | 8774 | CD1 | ILE | C | 690 | 29.702 | 80.126 | 41.285 | 1.00 | 34.94 | 6 |
| ATOM | 8775 | C | ILE | C | 690 | 27.260 | 77.523 | 44.144 | 1.00 | 13.87 | 6 |
| ATOM | 8776 | O | ILE | C | 690 | 27.636 | 76.695 | 44.955 | 1.00 | 13.87 | 8 |
| ATOM | 8777 | N | SER | C | 691 | 26.008 | 77.601 | 43.707 | 1.00 | 16.77 | 7 |
| ATOM | 8778 | CA | SER | C | 691 | 24.981 | 76.627 | 44.072 | 1.00 | 17.83 | 6 |
| ATOM | 8779 | CB | SER | C | 691 | 23.642 | 77.276 | 44.352 | 1.00 | 58.34 | 6 |
| ATOM | 8780 | OG | SER | C | 691 | 22.614 | 76.429 | 43.841 | 1.00 | 60.63 | 8 |
| ATOM | 8781 | C | SER | C | 691 | 24.816 | 75.815 | 42.805 | 1.00 | 17.19 | 6 |
| ATOM | 8782 | O | SER | C | 691 | 24.414 | 76.347 | 41.770 | 1.00 | 15.71 | 8 |
| ATOM | 8783 | N | GLU | C | 692 | 25.129 | 74.534 | 42.885 | 1.00 | 13.87 | 7 |
| ATOM | 8784 | CA | GLU | C | 692 | 25.048 | 73.667 | 41.729 | 1.00 | 15.29 | 6 |
| ATOM | 8785 | CB | GLU | C | 692 | 24.766 | 72.244 | 42.184 | 1.00 | 40.04 | 6 |
| ATOM | 8786 | CG | GLU | C | 692 | 24.620 | 71.239 | 41.067 | 1.00 | 42.87 | 6 |
| ATOM | 8787 | CD | GLU | C | 692 | 24.552 | 69.838 | 41.610 | 1.00 | 44.48 | 6 |
| ATOM | 8788 | OE1 | GLU | C | 692 | 23.552 | 69.515 | 42.291 | 1.00 | 44.52 | 8 |
| ATOM | 8789 | OE2 | GLU | C | 692 | 25.511 | 69.070 | 41.376 | 1.00 | 44.97 | 8 |
| ATOM | 8790 | C | GLU | C | 692 | 23.985 | 74.103 | 40.733 | 1.00 | 17.14 | 6 |
| ATOM | 8791 | O | GLU | C | 692 | 24.214 | 74.076 | 39.521 | 1.00 | 15.79 | 8 |
| ATOM | 8792 | N | GLU | C | 693 | 22.831 | 74.518 | 41.256 | 1.00 | 14.72 | 7 |
| ATOM | 8793 | CA | GLU | C | 693 | 21.709 | 74.923 | 40.422 | 1.00 | 17.95 | 6 |
| ATOM | 8794 | CB | GLU | C | 693 | 20.794 | 75.886 | 41.174 | 1.00 | 63.29 | 6 |
| ATOM | 8795 | CG | GLU | C | 693 | 19.366 | 75.867 | 40.666 | 1.00 | 67.04 | 6 |
| ATOM | 8796 | CD | GLU | C | 693 | 18.700 | 74.534 | 40.937 | 1.00 | 69.46 | 6 |
| ATOM | 8797 | OE1 | GLU | C | 693 | 19.240 | 73.494 | 40.511 | 1.00 | 70.03 | 8 |
| ATOM | 8798 | OE2 | GLU | C | 693 | 17.638 | 74.516 | 41.586 | 1.00 | 70.28 | 8 |
| ATOM | 8799 | C | GLU | C | 693 | 22.221 | 75.584 | 39.163 | 1.00 | 19.53 | 6 |
| ATOM | 8800 | O | GLU | C | 693 | 21.937 | 75.137 | 38.057 | 1.00 | 19.06 | 8 |
| ATOM | 8801 | N | LEU | C | 694 | 23.000 | 76.644 | 39.340 | 1.00 | 62.57 | 7 |
| ATOM | 8802 | CA | LEU | C | 694 | 23.547 | 77.362 | 38.206 | 1.00 | 65.09 | 6 |
| ATOM | 8803 | CB | LEU | C | 694 | 24.760 | 78.181 | 38.638 | 1.00 | 42.25 | 6 |
| ATOM | 8804 | CG | LEU | C | 694 | 24.343 | 79.563 | 39.117 | 1.00 | 42.71 | 6 |
| ATOM | 8805 | CD1 | LEU | C | 694 | 25.570 | 80.411 | 39.420 | 1.00 | 42.10 | 6 |
| ATOM | 8806 | CD2 | LEU | C | 694 | 23.490 | 80.205 | 38.027 | 1.00 | 41.62 | 6 |
| ATOM | 8807 | C | LEU | C | 694 | 23.927 | 76.394 | 37.108 | 1.00 | 66.05 | 6 |
| ATOM | 8808 | O | LEU | C | 694 | 23.478 | 76.515 | 35.966 | 1.00 | 67.07 | 8 |
| ATOM | 8809 | N | LEU | C | 695 | 24.744 | 75.415 | 37.462 | 1.00 | 16.69 | 7 |
| ATOM | 8810 | CA | LEU | C | 695 | 25.160 | 74.434 | 36.497 | 1.00 | 17.08 | 6 |
| ATOM | 8811 | CB | LEU | C | 695 | 26.129 | 73.458 | 37.146 | 1.00 | 22.44 | 6 |
| ATOM | 8812 | CG | LEU | C | 695 | 27.368 | 74.219 | 37.611 | 1.00 | 21.41 | 6 |
| ATOM | 8813 | CD1 | LEU | C | 695 | 28.391 | 73.268 | 38.217 | 1.00 | 20.97 | 6 |
| ATOM | 8814 | CD2 | LEU | C | 695 | 27.953 | 74.960 | 36.421 | 1.00 | 21.28 | 6 |
| ATOM | 8815 | C | LEU | C | 695 | 23.944 | 73.710 | 35.943 | 1.00 | 19.27 | 6 |
| ATOM | 8816 | O | LEU | C | 695 | 23.657 | 73.802 | 34.752 | 1.00 | 19.69 | 8 |
| ATOM | 8817 | N | LYS | C | 696 | 23.215 | 73.008 | 36.805 | 1.00 | 49.24 | 7 |
| ATOM | 8818 | CA | LYS | C | 696 | 22.035 | 72.270 | 36.367 | 1.00 | 50.88 | 6 |
| ATOM | 8819 | CB | LYS | C | 696 | 21.125 | 71.970 | 37.552 | 1.00 | 68.54 | 6 |
| ATOM | 8820 | CG | LYS | C | 696 | 21.725 | 71.037 | 38.581 | 1.00 | 70.86 | 6 |
| ATOM | 8821 | CD | LYS | C | 696 | 20.687 | 70.659 | 39.632 | 1.00 | 72.79 | 6 |
| ATOM | 8822 | CE | LYS | C | 696 | 21.212 | 69.583 | 40.572 | 1.00 | 74.27 | 6 |
| ATOM | 8823 | NZ | LYS | C | 696 | 20.177 | 69.068 | 41.512 | 1.00 | 74.29 | 7 |
| ATOM | 8824 | C | LYS | C | 696 | 21.244 | 73.007 | 35.288 | 1.00 | 51.81 | 6 |
| ATOM | 8825 | O | LYS | C | 696 | 20.695 | 72.375 | 34.388 | 1.00 | 51.36 | 8 |
| ATOM | 8826 | N | ARG | C | 697 | 21.194 | 74.337 | 35.362 | 1.00 | 55.92 | 7 |
| ATOM | 8827 | CA | ARG | C | 697 | 20.452 | 75.116 | 34.369 | 1.00 | 57.84 | 6 |
| ATOM | 8828 | CB | ARG | C | 697 | 19.665 | 76.260 | 35.031 | 1.00 | 98.96 | 6 |
| ATOM | 8829 | CG | ARG | C | 697 | 18.404 | 76.647 | 34.243 | 1.00 | 104.05 | 6 |
| ATOM | 8830 | CD | ARG | C | 697 | 17.797 | 77.996 | 34.653 | 1.00 | 107.95 | 6 |
| ATOM | 8831 | NE | ARG | C | 697 | 16.557 | 78.280 | 33.919 | 1.00 | 109.57 | 7 |
| ATOM | 8832 | CZ | ARG | C | 697 | 15.961 | 79.469 | 33.870 | 1.00 | 108.72 | 6 |
| ATOM | 8833 | NH1 | ARG | C | 697 | 16.486 | 80.508 | 34.505 | 1.00 | 109.06 | 7 |
| ATOM | 8834 | NH2 | ARG | C | 697 | 14.829 | 79.618 | 33.197 | 1.00 | 107.13 | 7 |
| ATOM | 8835 | C | ARG | C | 697 | 21.367 | 75.685 | 33.293 | 1.00 | 56.05 | 6 |
| ATOM | 8836 | O | ARG | C | 697 | 21.037 | 76.676 | 32.634 | 1.00 | 56.84 | 8 |
| ATOM | 8837 | N | ASP | C | 698 | 22.510 | 75.031 | 33.112 | 1.00 | 28.31 | 7 |
| ATOM | 8838 | CA | ASP | C | 698 | 23.501 | 75.437 | 32.123 | 1.00 | 25.94 | 6 |
| ATOM | 8839 | CB | ASP | C | 698 | 23.192 | 74.808 | 30.754 | 1.00 | 44.98 | 6 |
| ATOM | 8840 | CG | ASP | C | 698 | 24.071 | 73.593 | 30.449 | 1.00 | 46.56 | 6 |
| ATOM | 8841 | OD1 | ASP | C | 698 | 24.147 | 72.672 | 31.296 | 1.00 | 48.08 | 8 |
| ATOM | 8842 | OD2 | ASP | C | 698 | 24.682 | 73.556 | 29.359 | 1.00 | 46.15 | 8 |
| ATOM | 8843 | C | ASP | C | 698 | 23.577 | 76.948 | 32.004 | 1.00 | 24.33 | 6 |
| ATOM | 8844 | O | ASP | C | 698 | 23.360 | 77.512 | 30.930 | 1.00 | 24.07 | 8 |
| ATOM | 8845 | N | PHE | C | 699 | 23.889 | 77.598 | 33.121 | 1.00 | 75.64 | 7 |
| ATOM | 8846 | CA | PHE | C | 699 | 23.998 | 79.044 | 33.144 | 1.00 | 73.24 | 6 |
| ATOM | 8847 | CB | PHE | C | 699 | 23.801 | 79.581 | 34.557 | 1.00 | 40.01 | 6 |
| ATOM | 8848 | CG | PHE | C | 699 | 22.685 | 80.587 | 34.670 | 1.00 | 39.55 | 6 |
| ATOM | 8849 | CD1 | PHE | C | 699 | 21.392 | 80.260 | 34.279 | 1.00 | 38.54 | 6 |
| ATOM | 8850 | CD2 | PHE | C | 699 | 22.918 | 81.851 | 35.186 | 1.00 | 39.69 | 6 |
| ATOM | 8851 | CE1 | PHE | C | 699 | 20.358 | 81.166 | 34.403 | 1.00 | 36.47 | 6 |
| ATOM | 8852 | CE2 | PHE | C | 699 | 21.884 | 82.759 | 35.310 | 1.00 | 38.35 | 6 |
| ATOM | 8853 | CZ | PHE | C | 699 | 20.602 | 82.410 | 34.916 | 1.00 | 37.08 | 6 |
| ATOM | 8854 | C | PHE | C | 699 | 25.339 | 79.500 | 32.617 | 1.00 | 72.56 | 6 |
| ATOM | 8855 | O | PHE | C | 699 | 25.542 | 79.560 | 31.407 | 1.00 | 73.46 | 8 |
| ATOM | 8856 | N | TYR | C | 700 | 26.258 | 79.810 | 33.524 | 1.00 | 56.10 | 7 |
| ATOM | 8857 | CA | TYR | C | 700 | 27.577 | 80.289 | 33.140 | 1.00 | 54.08 | 6 |
| ATOM | 8858 | CB | TYR | C | 700 | 28.442 | 80.485 | 34.381 | 1.00 | 33.38 | 6 |
| ATOM | 8859 | CG | TYR | C | 700 | 27.977 | 81.655 | 35.201 | 1.00 | 31.13 | 6 |
| ATOM | 8860 | CD1 | TYR | C | 700 | 27.599 | 81.502 | 36.537 | 1.00 | 30.78 | 6 |
| ATOM | 8861 | CE1 | TYR | C | 700 | 27.068 | 82.577 | 37.255 | 1.00 | 28.77 | 6 |
| ATOM | 8862 | CD2 | TYR | C | 700 | 27.824 | 82.909 | 34.615 | 1.00 | 30.51 | 6 |
| ATOM | 8863 | CE2 | TYR | C | 700 | 27.297 | 83.979 | 35.324 | 1.00 | 28.57 | 6 |
| ATOM | 8864 | CZ | TYR | C | 700 | 26.920 | 83.803 | 36.628 | 1.00 | 27.48 | 6 |
| ATOM | 8865 | OH | TYR | C | 700 | 26.357 | 84.849 | 37.283 | 1.00 | 27.27 | 8 |
| ATOM | 8866 | C | TYR | C | 700 | 28.342 | 79.471 | 32.115 | 1.00 | 53.82 | 6 |
| ATOM | 8867 | O | TYR | C | 700 | 29.516 | 79.732 | 31.890 | 1.00 | 54.29 | 8 |
| ATOM | 8868 | N | THR | C | 701 | 27.694 | 78.497 | 31.484 | 1.00 | 39.84 | 7 |
| ATOM | 8869 | CA | THR | C | 701 | 28.366 | 77.689 | 30.472 | 1.00 | 39.95 | 6 |
| ATOM | 8870 | CB | THR | C | 701 | 27.471 | 76.562 | 29.956 | 1.00 | 20.05 | 6 |
| ATOM | 8871 | OG1 | THR | C | 701 | 26.281 | 77.124 | 29.393 | 1.00 | 19.30 | 8 |
| ATOM | 8872 | CG2 | THR | C | 701 | 27.117 | 75.610 | 31.086 | 1.00 | 19.72 | 6 |
| ATOM | 8873 | C | THR | C | 701 | 28.799 | 78.533 | 29.276 | 1.00 | 39.81 | 6 |
| ATOM | 8874 | O | THR | C | 701 | 28.035 | 79.360 | 28.760 | 1.00 | 37.90 | 8 |
| ATOM | 8875 | N | SER | C | 702 | 30.036 | 78.304 | 28.841 | 1.00 | 65.25 | 7 |
| ATOM | 8876 | CA | SER | C | 702 | 30.621 | 79.026 | 27.718 | 1.00 | 65.71 | 6 |
| ATOM | 8877 | CB | SER | C | 702 | 31.654 | 80.023 | 28.236 | 1.00 | 87.67 | 6 |
| ATOM | 8878 | OG | SER | C | 702 | 32.562 | 79.384 | 29.119 | 1.00 | 85.70 | 8 |
| ATOM | 8879 | C | SER | C | 702 | 31.285 | 78.059 | 26.748 | 1.00 | 66.12 | 6 |
| ATOM | 8880 | O | SER | C | 702 | 31.926 | 77.098 | 27.178 | 1.00 | 67.78 | 8 |
| ATOM | 8881 | N | ILE | C | 703 | 31.131 | 78.313 | 25.447 | 1.00 | 20.82 | 7 |
| ATOM | 8882 | CA | ILE | C | 703 | 31.718 | 77.448 | 24.419 | 1.00 | 20.77 | 6 |
| ATOM | 8883 | CB | ILE | C | 703 | 30.787 | 77.250 | 23.211 | 1.00 | 38.44 | 6 |
| ATOM | 8884 | CG2 | ILE | C | 703 | 31.537 | 76.534 | 22.109 | 1.00 | 39.40 | 6 |
| ATOM | 8885 | CG1 | ILE | C | 703 | 29.557 | 76.440 | 23.601 | 1.00 | 40.33 | 6 |
| ATOM | 8886 | CD1 | ILE | C | 703 | 28.480 | 76.440 | 22.529 | 1.00 | 40.31 | 6 |
| ATOM | 8887 | C | ILE | C | 703 | 32.999 | 78.042 | 23.877 | 1.00 | 19.96 | 6 |
| ATOM | 8888 | O | ILE | C | 703 | 33.104 | 79.246 | 23.693 | 1.00 | 19.42 | 8 |
| ATOM | 8889 | N | HIS | C | 704 | 33.965 | 77.182 | 23.605 | 1.00 | 38.89 | 7 |
| ATOM | 8890 | CA | HIS | C | 704 | 35.232 | 77.625 | 23.073 | 1.00 | 40.71 | 6 |
| ATOM | 8891 | CB | HIS | C | 704 | 36.270 | 77.734 | 24.188 | 1.00 | 79.40 | 6 |
| ATOM | 8892 | CG | HIS | C | 704 | 35.882 | 78.673 | 25.288 | 1.00 | 83.37 | 6 |
| ATOM | 8893 | CD2 | HIS | C | 704 | 36.288 | 79.934 | 25.570 | 1.00 | 85.44 | 6 |
| ATOM | 8894 | ND1 | HIS | C | 704 | 34.977 | 78.334 | 26.271 | 1.00 | 84.92 | 7 |
| ATOM | 8895 | CE1 | HIS | C | 704 | 34.847 | 79.344 | 27.113 | 1.00 | 85.50 | 6 |
| ATOM | 8896 | NE2 | HIS | C | 704 | 35.632 | 80.328 | 26.711 | 1.00 | 85.87 | 7 |
| ATOM | 8897 | C | HIS | C | 704 | 35.716 | 76.653 | 22.007 | 1.00 | 39.98 | 6 |
| ATOM | 8898 | O | HIS | C | 704 | 35.773 | 75.438 | 22.233 | 1.00 | 39.43 | 8 |
| ATOM | 8899 | N | ILE | C | 705 | 36.060 | 77.197 | 20.844 | 1.00 | 13.87 | 7 |
| ATOM | 8900 | CA | ILE | C | 705 | 36.543 | 76.389 | 19.746 | 1.00 | 13.87 | 6 |
| ATOM | 8901 | CB | ILE | C | 705 | 35.701 | 76.608 | 18.475 | 1.00 | 39.43 | 6 |
| ATOM | 8902 | CG2 | ILE | C | 705 | 35.011 | 75.330 | 18.084 | 1.00 | 40.68 | 6 |
| ATOM | 8903 | CG1 | ILE | C | 705 | 34.654 | 77.684 | 18.716 | 1.00 | 39.92 | 6 |
| ATOM | 8904 | CD1 | ILE | C | 705 | 35.261 | 79.040 | 18.825 | 1.00 | 43.09 | 6 |
| ATOM | 8905 | C | ILE | C | 705 | 37.986 | 76.723 | 19.439 | 1.00 | 13.87 | 6 |
| ATOM | 8906 | O | ILE | C | 705 | 38.247 | 77.786 | 18.903 | 1.00 | 13.87 | 8 |
| ATOM | 8907 | N | GLU | C | 706 | 38.905 | 75.811 | 19.785 | 1.00 | 32.47 | 7 |
| ATOM | 8908 | CA | GLU | C | 706 | 40.352 | 75.951 | 19.552 | 1.00 | 35.06 | 6 |
| ATOM | 8909 | CB | GLU | C | 706 | 41.130 | 75.086 | 20.545 | 1.00 | 76.65 | 6 |
| ATOM | 8910 | CG | GLU | C | 706 | 42.651 | 75.117 | 20.368 | 1.00 | 81.21 | 6 |
| ATOM | 8911 | CD | GLU | C | 706 | 43.317 | 76.323 | 21.016 | 1.00 | 83.25 | 6 |
| ATOM | 8912 | OE1 | GLU | C | 706 | 43.281 | 76.437 | 22.262 | 1.00 | 84.09 | 8 |
| ATOM | 8913 | OE2 | GLU | C | 706 | 43.885 | 77.157 | 20.279 | 1.00 | 83.41 | 8 |
| ATOM | 8914 | C | GLU | C | 706 | 40.718 | 75.524 | 18.121 | 1.00 | 35.08 | 6 |
| ATOM | 8915 | O | GLU | C | 706 | 39.916 | 74.867 | 17.446 | 1.00 | 35.64 | 8 |
| ATOM | 8916 | N | ARG | C | 707 | 41.923 | 75.896 | 17.672 | 1.00 | 30.19 | 7 |
| ATOM | 8917 | CA | ARG | C | 707 | 42.413 | 75.584 | 16.321 | 1.00 | 30.93 | 6 |
| ATOM | 8918 | CB | ARG | C | 707 | 42.520 | 76.849 | 15.482 | 1.00 | 53.21 | 6 |
| ATOM | 8919 | CG | ARG | C | 707 | 43.605 | 77.795 | 15.963 | 1.00 | 56.91 | 6 |
| ATOM | 8920 | CD | ARG | C | 707 | 43.655 | 79.070 | 15.149 | 1.00 | 61.47 | 6 |
| ATOM | 8921 | NE | ARG | C | 707 | 44.493 | 78.952 | 13.961 | 1.00 | 65.82 | 7 |
| ATOM | 8922 | CZ | ARG | C | 707 | 44.377 | 79.742 | 12.896 | 1.00 | 68.23 | 6 |
| ATOM | 8923 | NH1 | ARG | C | 707 | 43.455 | 80.697 | 12.875 | 1.00 | 68.81 | 7 |
| ATOM | 8924 | NH2 | ARG | C | 707 | 45.182 | 79.586 | 11.853 | 1.00 | 69.19 | 7 |
| ATOM | 8925 | C | ARG | C | 707 | 43.796 | 74.991 | 16.393 | 1.00 | 30.73 | 6 |
| ATOM | 8926 | O | ARG | C | 707 | 44.642 | 75.525 | 17.097 | 1.00 | 30.61 | 8 |
| ATOM | 8927 | N | TYR | C | 708 | 44.040 | 73.916 | 15.648 | 1.00 | 41.08 | 7 |
| ATOM | 8928 | CA | TYR | C | 708 | 45.353 | 73.271 | 15.649 | 1.00 | 42.17 | 6 |
| ATOM | 8929 | CB | TYR | C | 708 | 45.212 | 71.843 | 16.148 | 1.00 | 27.21 | 6 |
| ATOM | 8930 | CG | TYR | C | 708 | 44.688 | 71.803 | 17.549 | 1.00 | 24.62 | 6 |
| ATOM | 8931 | CD1 | TYR | C | 708 | 43.339 | 71.616 | 17.811 | 1.00 | 24.56 | 6 |
| ATOM | 8932 | CE1 | TYR | C | 708 | 42.867 | 71.642 | 19.108 | 1.00 | 25.28 | 6 |
| ATOM | 8933 | CD2 | TYR | C | 708 | 45.542 | 72.014 | 18.614 | 1.00 | 23.38 | 6 |
| ATOM | 8934 | CE2 | TYR | C | 708 | 45.085 | 72.046 | 19.906 | 1.00 | 23.76 | 6 |
| ATOM | 8935 | CZ | TYR | C | 708 | 43.758 | 71.858 | 20.156 | 1.00 | 25.23 | 6 |
| ATOM | 8936 | OH | TYR | C | 708 | 43.348 | 71.865 | 21.474 | 1.00 | 27.05 | 8 |
| ATOM | 8937 | C | TYR | C | 708 | 45.989 | 73.306 | 14.266 | 1.00 | 44.32 | 6 |
| ATOM | 8938 | O | TYR | C | 708 | 45.282 | 73.429 | 13.271 | 1.00 | 45.03 | 8 |
| ATOM | 8939 | N | GLU | C | 709 | 47.308 | 73.173 | 14.180 | 1.00 | 59.72 | 7 |
| ATOM | 8940 | CA | GLU | C | 709 | 47.932 | 73.269 | 12.870 | 1.00 | 62.76 | 6 |
| ATOM | 8941 | CB | GLU | C | 709 | 48.237 | 74.740 | 12.613 | 1.00 | 68.42 | 6 |
| ATOM | 8942 | CG | GLU | C | 709 | 48.738 | 75.053 | 11.231 | 1.00 | 71.85 | 6 |
| ATOM | 8943 | CD | GLU | C | 709 | 49.304 | 76.451 | 11.139 | 1.00 | 73.03 | 6 |
| ATOM | 8944 | OE1 | GLU | C | 709 | 50.381 | 76.692 | 11.733 | 1.00 | 74.70 | 8 |
| ATOM | 8945 | OE2 | GLU | C | 709 | 48.672 | 77.308 | 10.480 | 1.00 | 73.65 | 8 |
| ATOM | 8946 | C | GLU | C | 709 | 49.199 | 72.448 | 12.619 | 1.00 | 64.91 | 6 |
| ATOM | 8947 | O | GLU | C | 709 | 50.171 | 72.572 | 13.360 | 1.00 | 67.17 | 8 |
| ATOM | 8948 | N | ILE | C | 710 | 49.202 | 71.623 | 11.570 | 1.00 | 28.62 | 7 |
| ATOM | 8949 | CA | ILE | C | 710 | 50.392 | 70.833 | 11.233 | 1.00 | 29.55 | 6 |
| ATOM | 8950 | CB | ILE | C | 710 | 50.149 | 69.317 | 11.163 | 1.00 | 52.72 | 6 |
| ATOM | 8951 | CG2 | ILE | C | 710 | 51.454 | 68.583 | 11.256 | 1.00 | 50.81 | 6 |
| ATOM | 8952 | CG1 | ILE | C | 710 | 49.253 | 68.869 | 12.298 | 1.00 | 55.07 | 6 |
| ATOM | 8953 | CD1 | ILE | C | 710 | 47.826 | 69.266 | 12.079 | 1.00 | 58.93 | 6 |
| ATOM | 8954 | C | ILE | C | 710 | 50.744 | 71.246 | 9.833 | 1.00 | 30.82 | 6 |
| ATOM | 8955 | O | ILE | C | 710 | 49.868 | 71.319 | 8.973 | 1.00 | 30.33 | 8 |
| ATOM | 8956 | N | GLU | C | 711 | 52.024 | 71.515 | 9.605 | 1.00 | 72.44 | 7 |
| ATOM | 8957 | CA | GLU | C | 711 | 52.501 | 71.913 | 8.288 | 1.00 | 74.96 | 6 |
| ATOM | 8958 | CB | GLU | C | 711 | 53.389 | 73.162 | 8.387 | 1.00 | 90.20 | 6 |
| ATOM | 8959 | CG | GLU | C | 711 | 52.666 | 74.462 | 8.725 | 1.00 | 94.40 | 6 |
| ATOM | 8960 | CD | GLU | C | 711 | 53.627 | 75.600 | 9.069 | 1.00 | 97.08 | 6 |
| ATOM | 8961 | OE1 | GLU | C | 711 | 54.455 | 75.974 | 8.207 | 1.00 | 98.13 | 8 |
| ATOM | 8962 | OE2 | GLU | C | 711 | 53.553 | 76.120 | 10.208 | 1.00 | 97.93 | 8 |
| ATOM | 8963 | C | GLU | C | 711 | 53.310 | 70.774 | 7.676 | 1.00 | 75.37 | 6 |
| ATOM | 8964 | O | GLU | C | 711 | 54.193 | 70.211 | 8.325 | 1.00 | 75.49 | 8 |
| ATOM | 8965 | N | ALA | C | 712 | 52.991 | 70.433 | 6.430 | 1.00 | 25.69 | 7 |
| ATOM | 8966 | CA | ALA | C | 712 | 53.703 | 69.393 | 5.702 | 1.00 | 26.17 | 6 |
| ATOM | 8967 | CB | ALA | C | 712 | 52.773 | 68.741 | 4.709 | 1.00 | 74.02 | 6 |
| ATOM | 8968 | C | ALA | C | 712 | 54.897 | 70.036 | 4.978 | 1.00 | 27.04 | 6 |
| ATOM | 8969 | O | ALA | C | 712 | 54.767 | 70.553 | 3.866 | 1.00 | 26.68 | 8 |
| ATOM | 8970 | N | ARG | C | 713 | 56.053 | 70.003 | 5.636 | 1.00 | 51.20 | 7 |
| ATOM | 8971 | CA | ARG | C | 713 | 57.290 | 70.579 | 5.117 | 1.00 | 53.11 | 6 |
| ATOM | 8972 | CB | ARG | C | 713 | 58.366 | 70.539 | 6.207 | 1.00 | 108.41 | 6 |
| ATOM | 8973 | CG | ARG | C | 713 | 58.098 | 71.496 | 7.366 | 1.00 | 111.69 | 6 |
| ATOM | 8974 | CD | ARG | C | 713 | 58.397 | 72.927 | 6.942 | 1.00 | 114.40 | 6 |
| ATOM | 8975 | NE | ARG | C | 713 | 57.790 | 73.928 | 7.817 | 1.00 | 116.47 | 7 |
| ATOM | 8976 | CZ | ARG | C | 713 | 57.986 | 75.241 | 7.699 | 1.00 | 116.60 | 6 |
| ATOM | 8977 | NH1 | ARG | C | 713 | 58.780 | 75.715 | 6.744 | 1.00 | 116.86 | 7 |
| ATOM | 8978 | NH2 | ARG | C | 713 | 57.372 | 76.085 | 8.520 | 1.00 | 115.66 | 7 |
| ATOM | 8979 | C | ARG | C | 713 | 57.788 | 69.890 | 3.851 | 1.00 | 53.27 | 6 |
| ATOM | 8980 | O | ARG | C | 713 | 56.987 | 69.472 | 3.020 | 1.00 | 52.90 | 8 |
| ATOM | 8981 | N | ASP | C | 714 | 59.106 | 69.771 | 3.699 | 1.00 | 87.14 | 7 |
| ATOM | 8982 | CA | ASP | C | 714 | 59.674 | 69.145 | 2.502 | 1.00 | 87.63 | 6 |
| ATOM | 8983 | CB | ASP | C | 714 | 59.626 | 70.149 | 1.338 | 1.00 | 103.53 | 6 |
| ATOM | 8984 | CG | ASP | C | 714 | 59.136 | 69.529 | 0.035 | 1.00 | 104.91 | 6 |
| ATOM | 8985 | OD1 | ASP | C | 714 | 59.789 | 68.587 | −0.462 | 1.00 | 107.02 | 8 |
| ATOM | 8986 | OD2 | ASP | C | 714 | 58.099 | 69.989 | −0.497 | 1.00 | 105.51 | 8 |
| ATOM | 8987 | C | ASP | C | 714 | 61.118 | 68.643 | 2.695 | 1.00 | 86.74 | 6 |
| ATOM | 8988 | O | ASP | C | 714 | 61.704 | 68.070 | 1.780 | 1.00 | 86.18 | 8 |
| ATOM | 8989 | N | THR | C | 715 | 61.672 | 68.869 | 3.885 | 1.00 | 55.98 | 7 |
| ATOM | 8990 | CA | THR | C | 715 | 63.033 | 68.463 | 4.256 | 1.00 | 55.80 | 6 |
| ATOM | 8991 | CB | THR | C | 715 | 63.066 | 67.954 | 5.709 | 1.00 | 148.26 | 6 |
| ATOM | 8992 | OG1 | THR | C | 715 | 62.642 | 69.000 | 6.591 | 1.00 | 151.31 | 8 |
| ATOM | 8993 | CG2 | THR | C | 715 | 64.470 | 67.518 | 6.091 | 1.00 | 148.86 | 6 |
| ATOM | 8994 | C | THR | C | 715 | 63.689 | 67.403 | 3.371 | 1.00 | 54.30 | 6 |
| ATOM | 8995 | O | THR | C | 715 | 63.036 | 66.464 | 2.911 | 1.00 | 53.89 | 8 |
| ATOM | 8996 | N | LYS | C | 716 | 65.000 | 67.553 | 3.175 | 1.00 | 77.81 | 7 |
| ATOM | 8997 | CA | LYS | C | 716 | 65.806 | 66.660 | 2.331 | 1.00 | 76.91 | 6 |
| ATOM | 8998 | CB | LYS | C | 716 | 67.181 | 67.290 | 2.094 | 1.00 | 101.38 | 6 |
| ATOM | 8999 | CG | LYS | C | 716 | 67.121 | 68.775 | 1.775 | 1.00 | 103.28 | 6 |
| ATOM | 9000 | CD | LYS | C | 716 | 66.210 | 69.057 | 0.586 | 1.00 | 104.17 | 6 |
| ATOM | 9001 | CE | LYS | C | 716 | 66.006 | 70.551 | 0.378 | 1.00 | 104.81 | 6 |
| ATOM | 9002 | NZ | LYS | C | 716 | 65.012 | 70.813 | −0.697 | 1.00 | 104.29 | 7 |
| ATOM | 9003 | C | LYS | C | 716 | 65.996 | 65.230 | 2.837 | 1.00 | 75.08 | 6 |
| ATOM | 9004 | O | LYS | C | 716 | 67.114 | 64.726 | 2.906 | 1.00 | 75.31 | 8 |
| ATOM | 9005 | N | LEU | C | 717 | 64.896 | 64.574 | 3.171 | 1.00 | 36.58 | 7 |
| ATOM | 9006 | CA | LEU | C | 717 | 64.943 | 63.208 | 3.661 | 1.00 | 34.45 | 6 |
| ATOM | 9007 | CB | LEU | C | 717 | 64.889 | 63.195 | 5.197 | 1.00 | 47.94 | 6 |
| ATOM | 9008 | CG | LEU | C | 717 | 65.909 | 63.963 | 6.051 | 1.00 | 47.94 | 6 |
| ATOM | 9009 | CD1 | LEU | C | 717 | 65.231 | 64.441 | 7.322 | 1.00 | 47.23 | 6 |
| ATOM | 9010 | CD2 | LEU | C | 717 | 67.111 | 63.086 | 6.388 | 1.00 | 48.12 | 6 |
| ATOM | 9011 | C | LEU | C | 717 | 63.702 | 62.506 | 3.093 | 1.00 | 33.29 | 6 |
| ATOM | 9012 | O | LEU | C | 717 | 63.535 | 61.293 | 3.222 | 1.00 | 33.04 | 8 |
| ATOM | 9013 | N | GLY | C | 718 | 62.840 | 63.288 | 2.453 | 1.00 | 78.89 | 7 |
| ATOM | 9014 | CA | GLY | C | 718 | 61.616 | 62.750 | 1.890 | 1.00 | 77.32 | 6 |
| ATOM | 9015 | C | GLY | C | 718 | 60.473 | 63.676 | 2.266 | 1.00 | 76.02 | 6 |
| ATOM | 9016 | O | GLY | C | 718 | 60.290 | 63.967 | 3.451 | 1.00 | 77.03 | 8 |
| ATOM | 9017 | N | PRO | C | 719 | 59.677 | 64.143 | 1.289 | 1.00 | 28.75 | 7 |
| ATOM | 9018 | CD | PRO | C | 719 | 59.532 | 63.487 | −0.017 | 1.00 | 61.22 | 6 |
| ATOM | 9019 | CA | PRO | C | 719 | 58.551 | 65.047 | 1.517 | 1.00 | 26.52 | 6 |
| ATOM | 9020 | CB | PRO | C | 719 | 57.773 | 64.939 | 0.218 | 1.00 | 60.27 | 6 |
| ATOM | 9021 | CG | PRO | C | 719 | 58.052 | 63.544 | −0.211 | 1.00 | 61.08 | 6 |
| ATOM | 9022 | C | PRO | C | 719 | 57.705 | 64.679 | 2.730 | 1.00 | 24.65 | 6 |
| ATOM | 9023 | O | PRO | C | 719 | 57.291 | 63.528 | 2.888 | 1.00 | 23.98 | 8 |
| ATOM | 9024 | N | GLU | C | 720 | 57.449 | 65.675 | 3.575 | 1.00 | 74.17 | 7 |
| ATOM | 9025 | CA | GLU | C | 720 | 56.654 | 65.515 | 4.791 | 1.00 | 72.87 | 6 |
| ATOM | 9026 | CB | GLU | C | 720 | 56.977 | 66.683 | 5.731 | 1.00 | 33.81 | 6 |
| ATOM | 9027 | CG | GLU | C | 720 | 56.510 | 66.482 | 7.150 | 1.00 | 34.95 | 6 |
| ATOM | 9028 | CD | GLU | C | 720 | 57.247 | 67.350 | 8.159 | 1.00 | 35.85 | 6 |
| ATOM | 9029 | OE1 | GLU | C | 720 | 57.315 | 68.583 | 7.975 | 1.00 | 37.31 | 8 |
| ATOM | 9030 | OE2 | GLU | C | 720 | 57.753 | 66.797 | 9.158 | 1.00 | 37.14 | 8 |
| ATOM | 9031 | C | GLU | C | 720 | 55.172 | 65.519 | 4.395 | 1.00 | 72.23 | 6 |
| ATOM | 9032 | O | GLU | C | 720 | 54.634 | 66.578 | 4.100 | 1.00 | 73.55 | 8 |
| ATOM | 9033 | N | ARG | C | 721 | 54.508 | 64.359 | 4.402 | 1.00 | 13.87 | 7 |
| ATOM | 9034 | CA | ARG | C | 721 | 53.105 | 64.293 | 3.964 | 1.00 | 13.87 | 6 |
| ATOM | 9035 | CB | ARG | C | 721 | 53.058 | 63.421 | 2.712 | 1.00 | 78.27 | 6 |
| ATOM | 9036 | CG | ARG | C | 721 | 54.048 | 62.262 | 2.741 | 1.00 | 82.81 | 6 |
| ATOM | 9037 | CD | ARG | C | 721 | 54.002 | 61.436 | 1.459 | 1.00 | 85.00 | 6 |
| ATOM | 9038 | NE | ARG | C | 721 | 54.864 | 60.258 | 1.533 | 1.00 | 87.32 | 7 |
| ATOM | 9039 | CZ | ARG | C | 721 | 54.912 | 59.309 | 0.603 | 1.00 | 88.92 | 6 |
| ATOM | 9040 | NH1 | ARG | C | 721 | 54.143 | 59.402 | −0.473 | 1.00 | 90.34 | 7 |
| ATOM | 9041 | NH2 | ARG | C | 721 | 55.727 | 58.270 | 0.743 | 1.00 | 89.06 | 7 |
| ATOM | 9042 | C | ARG | C | 721 | 51.944 | 63.891 | 4.917 | 1.00 | 13.87 | 6 |
| ATOM | 9043 | O | ARG | C | 721 | 52.069 | 62.980 | 5.723 | 1.00 | 13.87 | 8 |
| ATOM | 9044 | N | ILE | C | 722 | 50.807 | 64.589 | 4.782 | 1.00 | 55.68 | 7 |
| ATOM | 9045 | CA | ILE | C | 722 | 49.577 | 64.366 | 5.574 | 1.00 | 55.53 | 6 |
| ATOM | 9046 | CB | ILE | C | 722 | 48.592 | 65.591 | 5.476 | 1.00 | 24.95 | 6 |
| ATOM | 9047 | CG2 | ILE | C | 722 | 47.231 | 65.222 | 5.956 | 1.00 | 24.08 | 6 |
| ATOM | 9048 | CG1 | ILE | C | 722 | 49.086 | 66.757 | 6.315 | 1.00 | 22.72 | 6 |
| ATOM | 9049 | CD1 | ILE | C | 722 | 49.644 | 67.910 | 5.495 | 1.00 | 22.52 | 6 |
| ATOM | 9050 | C | ILE | C | 722 | 48.833 | 63.141 | 5.037 | 1.00 | 56.81 | 6 |
| ATOM | 9051 | O | ILE | C | 722 | 47.989 | 63.269 | 4.147 | 1.00 | 58.37 | 8 |
| ATOM | 9052 | N | THR | C | 723 | 49.129 | 61.966 | 5.589 | 1.00 | 28.07 | 7 |
| ATOM | 9053 | CA | THR | C | 723 | 48.520 | 60.704 | 5.142 | 1.00 | 28.92 | 6 |
| ATOM | 9054 | CB | THR | C | 723 | 49.590 | 59.663 | 4.793 | 1.00 | 55.19 | 6 |
| ATOM | 9055 | OG1 | THR | C | 723 | 49.707 | 58.734 | 5.878 | 1.00 | 54.66 | 8 |
| ATOM | 9056 | CG2 | THR | C | 723 | 50.943 | 60.329 | 4.592 | 1.00 | 56.20 | 6 |
| ATOM | 9057 | C | THR | C | 723 | 47.687 | 60.061 | 6.227 | 1.00 | 29.52 | 6 |
| ATOM | 9058 | O | THR | C | 723 | 47.771 | 60.438 | 7.392 | 1.00 | 29.63 | 8 |
| ATOM | 9059 | N | ALA | C | 724 | 46.893 | 59.069 | 5.856 | 1.00 | 33.12 | 7 |
| ATOM | 9060 | CA | ALA | C | 724 | 46.100 | 58.364 | 6.857 | 1.00 | 35.05 | 6 |
| ATOM | 9061 | CB | ALA | C | 724 | 44.668 | 58.166 | 6.377 | 1.00 | 81.81 | 6 |
| ATOM | 9062 | C | ALA | C | 724 | 46.776 | 57.012 | 7.110 | 1.00 | 35.95 | 6 |
| ATOM | 9063 | O | ALA | C | 724 | 46.376 | 56.265 | 8.003 | 1.00 | 35.14 | 8 |
| ATOM | 9064 | N | ASP | C | 725 | 47.813 | 56.713 | 6.325 | 1.00 | 48.22 | 7 |
| ATOM | 9065 | CA | ASP | C | 725 | 48.562 | 55.462 | 6.465 | 1.00 | 50.41 | 6 |
| ATOM | 9066 | CB | ASP | C | 725 | 49.056 | 54.969 | 5.096 | 1.00 | 135.83 | 6 |
| ATOM | 9067 | CG | ASP | C | 725 | 49.925 | 55.994 | 4.368 | 1.00 | 140.29 | 6 |
| ATOM | 9068 | OD1 | ASP | C | 725 | 50.925 | 56.472 | 4.945 | 1.00 | 142.55 | 8 |
| ATOM | 9069 | OD2 | ASP | C | 725 | 49.612 | 56.313 | 3.202 | 1.00 | 142.25 | 8 |
| ATOM | 9070 | C | ASP | C | 725 | 49.753 | 55.649 | 7.401 | 1.00 | 50.45 | 6 |
| ATOM | 9071 | O | ASP | C | 725 | 50.905 | 55.434 | 7.018 | 1.00 | 49.56 | 8 |
| ATOM | 9072 | N | ILE | C | 726 | 49.485 | 56.040 | 8.639 | 1.00 | 18.04 | 7 |
| ATOM | 9073 | CA | ILE | C | 726 | 50.586 | 56.249 | 9.564 | 1.00 | 19.68 | 6 |
| ATOM | 9074 | CB | ILE | C | 726 | 50.171 | 57.123 | 10.759 | 1.00 | 99.69 | 6 |
| ATOM | 9075 | CG2 | ILE | C | 726 | 51.412 | 57.681 | 11.436 | 1.00 | 100.55 | 6 |
| ATOM | 9076 | CG1 | ILE | C | 726 | 49.311 | 58.296 | 10.282 | 1.00 | 100.68 | 6 |
| ATOM | 9077 | CD1 | ILE | C | 726 | 47.898 | 57.913 | 9.923 | 1.00 | 103.55 | 6 |
| ATOM | 9078 | C | ILE | C | 726 | 51.108 | 54.911 | 10.068 | 1.00 | 20.10 | 6 |
| ATOM | 9079 | O | ILE | C | 726 | 50.345 | 54.066 | 10.531 | 1.00 | 18.78 | 8 |
| ATOM | 9080 | N | PRO | C | 727 | 52.427 | 54.708 | 9.983 | 1.00 | 49.83 | 7 |
| ATOM | 9081 | CD | PRO | C | 727 | 53.409 | 55.723 | 9.573 | 1.00 | 39.86 | 6 |
| ATOM | 9082 | CA | PRO | C | 727 | 53.111 | 53.489 | 10.409 | 1.00 | 52.26 | 6 |
| ATOM | 9083 | CB | PRO | C | 727 | 54.585 | 53.839 | 10.191 | 1.00 | 41.13 | 6 |
| ATOM | 9084 | CG | PRO | C | 727 | 54.619 | 55.306 | 10.355 | 1.00 | 40.83 | 6 |
| ATOM | 9085 | C | PRO | C | 727 | 52.833 | 52.945 | 11.819 | 1.00 | 54.67 | 6 |
| ATOM | 9086 | O | PRO | C | 727 | 52.907 | 53.671 | 12.813 | 1.00 | 54.95 | 8 |
| ATOM | 9087 | N | HIS | C | 728 | 52.523 | 51.650 | 11.877 | 1.00 | 103.53 | 7 |
| ATOM | 9088 | CA | HIS | C | 728 | 52.253 | 50.922 | 13.119 | 1.00 | 106.42 | 6 |
| ATOM | 9089 | CB | HIS | C | 728 | 53.554 | 50.696 | 13.892 | 1.00 | 81.73 | 6 |
| ATOM | 9090 | CG | HIS | C | 728 | 54.724 | 50.396 | 13.011 | 1.00 | 82.69 | 6 |
| ATOM | 9091 | CD2 | HIS | C | 728 | 56.024 | 50.764 | 13.100 | 1.00 | 82.73 | 6 |
| ATOM | 9092 | ND1 | HIS | C | 728 | 54.613 | 49.654 | 11.854 | 1.00 | 83.03 | 7 |
| ATOM | 9093 | CE1 | HIS | C | 728 | 55.794 | 49.581 | 11.266 | 1.00 | 83.77 | 6 |
| ATOM | 9094 | NE2 | HIS | C | 728 | 56.668 | 50.246 | 12.001 | 1.00 | 83.57 | 7 |
| ATOM | 9095 | C | HIS | C | 728 | 51.229 | 51.570 | 14.034 | 1.00 | 107.81 | 6 |
| ATOM | 9096 | O | HIS | C | 728 | 50.869 | 51.013 | 15.072 | 1.00 | 107.76 | 8 |
| ATOM | 9097 | N | LEU | C | 729 | 50.770 | 52.754 | 13.652 | 1.00 | 67.78 | 7 |
| ATOM | 9098 | CA | LEU | C | 729 | 49.761 | 53.460 | 14.422 | 1.00 | 68.12 | 6 |
| ATOM | 9099 | CB | LEU | C | 729 | 49.593 | 54.869 | 13.860 | 1.00 | 89.16 | 6 |
| ATOM | 9100 | CG | LEU | C | 729 | 48.724 | 55.863 | 14.621 | 1.00 | 90.78 | 6 |
| ATOM | 9101 | CD1 | LEU | C | 729 | 49.227 | 55.984 | 16.045 | 1.00 | 92.34 | 6 |
| ATOM | 9102 | CD2 | LEU | C | 729 | 48.759 | 57.211 | 13.917 | 1.00 | 91.03 | 6 |
| ATOM | 9103 | C | LEU | C | 729 | 48.500 | 52.632 | 14.191 | 1.00 | 68.16 | 6 |
| ATOM | 9104 | O | LEU | C | 729 | 47.477 | 52.824 | 14.839 | 1.00 | 67.48 | 8 |
| ATOM | 9105 | N | SER | C | 730 | 48.626 | 51.699 | 13.250 | 1.00 | 37.40 | 7 |
| ATOM | 9106 | CA | SER | C | 730 | 47.581 | 50.780 | 12.829 | 1.00 | 39.19 | 6 |
| ATOM | 9107 | CB | SER | C | 730 | 48.182 | 49.397 | 12.573 | 1.00 | 171.68 | 6 |
| ATOM | 9108 | OG | SER | C | 730 | 48.748 | 48.864 | 13.757 | 1.00 | 174.99 | 8 |
| ATOM | 9109 | C | SER | C | 730 | 46.403 | 50.643 | 13.769 | 1.00 | 39.37 | 6 |
| ATOM | 9110 | O | SER | C | 730 | 45.267 | 50.490 | 13.311 | 1.00 | 38.41 | 8 |
| ATOM | 9111 | N | GLU | C | 731 | 46.662 | 50.666 | 15.073 | 1.00 | 87.98 | 7 |
| ATOM | 9112 | CA | GLU | C | 731 | 45.581 | 50.559 | 16.043 | 1.00 | 89.62 | 6 |
| ATOM | 9113 | CB | GLU | C | 731 | 45.897 | 51.360 | 17.311 | 1.00 | 85.62 | 6 |
| ATOM | 9114 | CG | GLU | C | 731 | 46.841 | 50.716 | 18.314 | 1.00 | 86.23 | 6 |
| ATOM | 9115 | CD | GLU | C | 731 | 46.972 | 51.563 | 19.565 | 1.00 | 86.43 | 6 |
| ATOM | 9116 | OE1 | GLU | C | 731 | 45.922 | 51.873 | 20.167 | 1.00 | 86.77 | 8 |
| ATOM | 9117 | OE2 | GLU | C | 731 | 48.111 | 51.924 | 19.939 | 1.00 | 86.84 | 8 |
| ATOM | 9118 | C | GLU | C | 731 | 44.335 | 51.150 | 15.407 | 1.00 | 90.42 | 6 |
| ATOM | 9119 | O | GLU | C | 731 | 44.418 | 52.135 | 14.680 | 1.00 | 91.43 | 8 |
| ATOM | 9120 | N | ALA | C | 732 | 43.181 | 50.552 | 15.651 | 1.00 | 65.70 | 7 |
| ATOM | 9121 | CA | ALA | C | 732 | 41.965 | 51.102 | 15.085 | 1.00 | 66.50 | 6 |
| ATOM | 9122 | CB | ALA | C | 732 | 40.777 | 50.219 | 15.428 | 1.00 | 161.43 | 6 |
| ATOM | 9123 | C | ALA | C | 732 | 41.793 | 52.484 | 15.705 | 1.00 | 66.79 | 6 |
| ATOM | 9124 | O | ALA | C | 732 | 41.086 | 52.641 | 16.700 | 1.00 | 67.04 | 8 |
| ATOM | 9125 | N | ALA | C | 733 | 42.481 | 53.467 | 15.126 | 1.00 | 50.23 | 7 |
| ATOM | 9126 | CA | ALA | C | 733 | 42.444 | 54.865 | 15.557 | 1.00 | 49.45 | 6 |
| ATOM | 9127 | CB | ALA | C | 733 | 43.731 | 55.252 | 16.247 | 1.00 | 13.87 | 6 |
| ATOM | 9128 | C | ALA | C | 733 | 42.352 | 55.588 | 14.248 | 1.00 | 49.60 | 6 |
| ATOM | 9129 | O | ALA | C | 733 | 41.327 | 56.170 | 13.900 | 1.00 | 49.80 | 8 |
| ATOM | 9130 | N | LEU | C | 734 | 43.459 | 55.514 | 13.525 | 1.00 | 61.31 | 7 |
| ATOM | 9131 | CA | LEU | C | 734 | 43.614 | 56.108 | 12.206 | 1.00 | 62.40 | 6 |
| ATOM | 9132 | CB | LEU | C | 734 | 44.379 | 55.138 | 11.312 | 1.00 | 67.95 | 6 |
| ATOM | 9133 | CG | LEU | C | 734 | 45.512 | 54.422 | 12.034 | 1.00 | 68.37 | 6 |
| ATOM | 9134 | CD1 | LEU | C | 734 | 45.858 | 53.168 | 11.259 | 1.00 | 69.37 | 6 |
| ATOM | 9135 | CD2 | LEU | C | 734 | 46.704 | 55.357 | 12.199 | 1.00 | 67.78 | 6 |
| ATOM | 9136 | C | LEU | C | 734 | 42.274 | 56.402 | 11.560 | 1.00 | 62.65 | 6 |
| ATOM | 9137 | O | LEU | C | 734 | 41.699 | 57.478 | 11.743 | 1.00 | 62.31 | 8 |
| ATOM | 9138 | N | ARG | C | 735 | 41.800 | 55.415 | 10.805 | 1.00 | 146.73 | 7 |
| ATOM | 9139 | CA | ARG | C | 735 | 40.539 | 55.495 | 10.091 | 1.00 | 146.90 | 6 |
| ATOM | 9140 | CB | ARG | C | 735 | 40.562 | 54.540 | 8.892 | 1.00 | 160.15 | 6 |
| ATOM | 9141 | CG | ARG | C | 735 | 41.615 | 54.913 | 7.849 | 1.00 | 162.75 | 6 |
| ATOM | 9142 | CD | ARG | C | 735 | 41.786 | 53.827 | 6.801 | 1.00 | 166.69 | 6 |
| ATOM | 9143 | NE | ARG | C | 735 | 42.920 | 54.096 | 5.918 | 1.00 | 169.50 | 7 |
| ATOM | 9144 | CZ | ARG | C | 735 | 43.435 | 53.210 | 5.067 | 1.00 | 170.43 | 6 |
| ATOM | 9145 | NH1 | ARG | C | 735 | 42.919 | 51.989 | 4.981 | 1.00 | 171.49 | 7 |
| ATOM | 9146 | NH2 | ARG | C | 735 | 44.467 | 53.542 | 4.302 | 1.00 | 170.03 | 7 |
| ATOM | 9147 | C | ARG | C | 735 | 39.349 | 55.203 | 11.002 | 1.00 | 145.75 | 6 |
| ATOM | 9148 | O | ARG | C | 735 | 38.202 | 55.181 | 10.549 | 1.00 | 146.80 | 8 |
| ATOM | 9149 | N | ASP | C | 736 | 39.621 | 54.972 | 12.286 | 1.00 | 94.65 | 7 |
| ATOM | 9150 | CA | ASP | C | 736 | 38.541 | 54.738 | 13.242 | 1.00 | 92.16 | 6 |
| ATOM | 9151 | CB | ASP | C | 736 | 39.091 | 54.274 | 14.598 | 1.00 | 181.71 | 6 |
| ATOM | 9152 | CG | ASP | C | 736 | 38.028 | 53.604 | 15.467 | 1.00 | 184.26 | 6 |
| ATOM | 9153 | OD1 | ASP | C | 736 | 37.045 | 54.276 | 15.841 | 1.00 | 185.06 | 8 |
| ATOM | 9154 | OD2 | ASP | C | 736 | 38.177 | 52.401 | 15.775 | 1.00 | 184.92 | 8 |
| ATOM | 9155 | C | ASP | C | 736 | 37.965 | 56.145 | 13.334 | 1.00 | 88.95 | 6 |
| ATOM | 9156 | O | ASP | C | 736 | 36.883 | 56.382 | 13.860 | 1.00 | 88.81 | 8 |
| ATOM | 9157 | N | LEU | C | 737 | 38.742 | 57.067 | 12.785 | 1.00 | 49.70 | 7 |
| ATOM | 9158 | CA | LEU | C | 737 | 38.427 | 58.479 | 12.688 | 1.00 | 46.17 | 6 |
| ATOM | 9159 | CB | LEU | C | 737 | 38.811 | 59.197 | 13.971 | 1.00 | 66.95 | 6 |
| ATOM | 9160 | CG | LEU | C | 737 | 38.021 | 58.770 | 15.203 | 1.00 | 67.44 | 6 |
| ATOM | 9161 | CD1 | LEU | C | 737 | 38.541 | 59.520 | 16.433 | 1.00 | 67.94 | 6 |
| ATOM | 9162 | CD2 | LEU | C | 737 | 36.539 | 59.037 | 14.962 | 1.00 | 67.08 | 6 |
| ATOM | 9163 | C | LEU | C | 737 | 39.361 | 58.876 | 11.556 | 1.00 | 43.59 | 6 |
| ATOM | 9164 | O | LEU | C | 737 | 39.724 | 58.036 | 10.745 | 1.00 | 43.12 | 8 |
| ATOM | 9165 | N | ALA | C | 738 | 39.747 | 60.136 | 11.478 | 1.00 | 51.25 | 7 |
| ATOM | 9166 | CA | ALA | C | 738 | 40.673 | 60.534 | 10.434 | 1.00 | 48.98 | 6 |
| ATOM | 9167 | CB | ALA | C | 738 | 42.075 | 60.088 | 10.823 | 1.00 | 13.87 | 6 |
| ATOM | 9168 | C | ALA | C | 738 | 40.307 | 59.926 | 9.090 | 1.00 | 47.86 | 6 |
| ATOM | 9169 | O | ALA | C | 738 | 40.893 | 58.938 | 8.704 | 1.00 | 47.89 | 8 |
| ATOM | 9170 | N | GLU | C | 739 | 39.348 | 60.496 | 8.376 | 1.00 | 27.31 | 7 |
| ATOM | 9171 | CA | GLU | C | 739 | 38.975 | 59.959 | 7.072 | 1.00 | 27.39 | 6 |
| ATOM | 9172 | CB | GLU | C | 739 | 37.475 | 60.158 | 6.801 | 1.00 | 132.71 | 6 |
| ATOM | 9173 | CG | GLU | C | 739 | 37.062 | 61.524 | 6.264 | 1.00 | 136.99 | 6 |
| ATOM | 9174 | CD | GLU | C | 739 | 36.706 | 61.491 | 4.785 | 1.00 | 139.07 | 6 |
| ATOM | 9175 | OE1 | GLU | C | 739 | 35.918 | 60.610 | 4.376 | 1.00 | 139.76 | 8 |
| ATOM | 9176 | OE2 | GLU | C | 739 | 37.204 | 62.355 | 4.033 | 1.00 | 140.52 | 8 |
| ATOM | 9177 | C | GLU | C | 739 | 39.815 | 60.646 | 6.002 | 1.00 | 26.59 | 6 |
| ATOM | 9178 | O | GLU | C | 739 | 39.869 | 61.886 | 5.938 | 1.00 | 24.52 | 8 |
| ATOM | 9179 | N | GLU | C | 740 | 40.476 | 59.813 | 5.189 | 1.00 | 87.32 | 7 |
| ATOM | 9180 | CA | GLU | C | 740 | 41.383 | 60.209 | 4.098 | 1.00 | 87.44 | 6 |
| ATOM | 9181 | CB | GLU | C | 740 | 40.601 | 60.823 | 2.923 | 1.00 | 93.01 | 6 |
| ATOM | 9182 | CG | GLU | C | 740 | 39.903 | 59.786 | 2.032 | 1.00 | 95.24 | 6 |
| ATOM | 9183 | CD | GLU | C | 740 | 39.841 | 60.197 | 0.555 | 1.00 | 97.00 | 6 |
| ATOM | 9184 | OE1 | GLU | C | 740 | 40.909 | 60.453 | −0.046 | 1.00 | 96.19 | 8 |
| ATOM | 9185 | OE2 | GLU | C | 740 | 38.727 | 60.255 | −0.014 | 1.00 | 99.01 | 8 |
| ATOM | 9186 | C | GLU | C | 740 | 42.541 | 61.138 | 4.513 | 1.00 | 86.01 | 6 |
| ATOM | 9187 | O | GLU | C | 740 | 43.079 | 61.899 | 3.698 | 1.00 | 85.82 | 8 |
| ATOM | 9188 | N | GLY | C | 741 | 42.932 | 61.046 | 5.784 | 1.00 | 68.95 | 7 |
| ATOM | 9189 | CA | GLY | C | 741 | 44.013 | 61.865 | 6.305 | 1.00 | 65.05 | 6 |
| ATOM | 9190 | C | GLY | C | 741 | 43.694 | 62.646 | 7.577 | 1.00 | 62.52 | 6 |
| ATOM | 9191 | O | GLY | C | 741 | 44.281 | 62.406 | 8.632 | 1.00 | 62.09 | 8 |
| ATOM | 9192 | N | ILE | C | 742 | 42.750 | 63.573 | 7.480 | 1.00 | 68.13 | 7 |
| ATOM | 9193 | CA | ILE | C | 742 | 42.373 | 64.422 | 8.604 | 1.00 | 67.23 | 6 |
| ATOM | 9194 | CB | ILE | C | 742 | 42.153 | 65.846 | 8.094 | 1.00 | 44.50 | 6 |
| ATOM | 9195 | CG2 | ILE | C | 742 | 41.758 | 66.753 | 9.243 | 1.00 | 44.68 | 6 |
| ATOM | 9196 | CG1 | ILE | C | 742 | 43.417 | 66.337 | 7.379 | 1.00 | 44.07 | 6 |
| ATOM | 9197 | CD1 | ILE | C | 742 | 43.210 | 67.589 | 6.540 | 1.00 | 46.03 | 6 |
| ATOM | 9198 | C | ILE | C | 742 | 41.113 | 63.967 | 9.352 | 1.00 | 66.56 | 6 |
| ATOM | 9199 | O | ILE | C | 742 | 40.197 | 63.423 | 8.737 | 1.00 | 67.84 | 8 |
| ATOM | 9200 | N | VAL | C | 743 | 41.075 | 64.201 | 10.669 | 1.00 | 34.97 | 7 |
| ATOM | 9201 | CA | VAL | C | 743 | 39.929 | 63.838 | 11.517 | 1.00 | 31.92 | 6 |
| ATOM | 9202 | CB | VAL | C | 743 | 40.014 | 64.506 | 12.923 | 1.00 | 31.40 | 6 |
| ATOM | 9203 | CG1 | VAL | C | 743 | 38.798 | 65.361 | 13.184 | 1.00 | 30.37 | 6 |
| ATOM | 9204 | CG2 | VAL | C | 743 | 40.110 | 63.468 | 13.999 | 1.00 | 30.39 | 6 |
| ATOM | 9205 | C | VAL | C | 743 | 38.657 | 64.322 | 10.841 | 1.00 | 31.20 | 6 |
| ATOM | 9206 | O | VAL | C | 743 | 38.699 | 65.219 | 9.997 | 1.00 | 30.47 | 8 |
| ATOM | 9207 | N | ALA | C | 744 | 37.523 | 63.744 | 11.210 | 1.00 | 53.96 | 7 |
| ATOM | 9208 | CA | ALA | C | 744 | 36.256 | 64.150 | 10.607 | 1.00 | 54.41 | 6 |
| ATOM | 9209 | CB | ALA | C | 744 | 35.362 | 62.927 | 10.407 | 1.00 | 103.76 | 6 |
| ATOM | 9210 | C | ALA | C | 744 | 35.534 | 65.197 | 11.454 | 1.00 | 53.29 | 6 |
| ATOM | 9211 | O | ALA | C | 744 | 36.020 | 65.581 | 12.519 | 1.00 | 53.58 | 8 |
| ATOM | 9212 | N | ILE | C | 745 | 34.381 | 65.663 | 10.978 | 1.00 | 16.77 | 7 |
| ATOM | 9213 | CA | ILE | C | 745 | 33.620 | 66.653 | 11.728 | 1.00 | 13.90 | 6 |
| ATOM | 9214 | CB | ILE | C | 745 | 32.634 | 67.422 | 10.852 | 1.00 | 35.23 | 6 |
| ATOM | 9215 | CG2 | ILE | C | 745 | 32.013 | 68.549 | 11.667 | 1.00 | 33.55 | 6 |
| ATOM | 9216 | CG1 | ILE | C | 745 | 33.354 | 67.995 | 9.634 | 1.00 | 35.74 | 6 |
| ATOM | 9217 | CD1 | ILE | C | 745 | 34.431 | 68.987 | 9.992 | 1.00 | 36.65 | 6 |
| ATOM | 9218 | C | ILE | C | 745 | 32.836 | 65.918 | 12.792 | 1.00 | 13.87 | 6 |
| ATOM | 9219 | O | ILE | C | 745 | 32.599 | 64.723 | 12.672 | 1.00 | 13.87 | 8 |
| ATOM | 9220 | N | GLY | C | 746 | 32.448 | 66.639 | 13.836 | 1.00 | 42.04 | 7 |
| ATOM | 9221 | CA | GLY | C | 746 | 31.702 | 66.034 | 14.925 | 1.00 | 43.25 | 6 |
| ATOM | 9222 | C | GLY | C | 746 | 32.507 | 64.934 | 15.577 | 1.00 | 43.26 | 6 |
| ATOM | 9223 | O | GLY | C | 746 | 32.074 | 64.275 | 16.523 | 1.00 | 42.12 | 8 |
| ATOM | 9224 | N | ALA | C | 747 | 33.709 | 64.746 | 15.066 | 1.00 | 32.22 | 7 |
| ATOM | 9225 | CA | ALA | C | 747 | 34.560 | 63.709 | 15.587 | 1.00 | 33.91 | 6 |
| ATOM | 9226 | CB | ALA | C | 747 | 35.754 | 63.542 | 14.682 | 1.00 | 37.39 | 6 |
| ATOM | 9227 | C | ALA | C | 747 | 35.012 | 64.005 | 17.012 | 1.00 | 35.07 | 6 |
| ATOM | 9228 | O | ALA | C | 747 | 36.157 | 64.414 | 17.226 | 1.00 | 35.92 | 8 |
| ATOM | 9229 | N | GLU | C | 748 | 34.135 | 63.800 | 17.994 | 1.00 | 22.58 | 7 |
| ATOM | 9230 | CA | GLU | C | 748 | 34.531 | 64.064 | 19.376 | 1.00 | 23.16 | 6 |
| ATOM | 9231 | CB | GLU | C | 748 | 33.567 | 63.386 | 20.343 | 1.00 | 71.83 | 6 |
| ATOM | 9232 | CG | GLU | C | 748 | 33.923 | 63.604 | 21.789 | 1.00 | 74.78 | 6 |
| ATOM | 9233 | CD | GLU | C | 748 | 35.057 | 62.711 | 22.235 | 1.00 | 75.97 | 6 |
| ATOM | 9234 | OE1 | GLU | C | 748 | 35.635 | 62.969 | 23.317 | 1.00 | 76.61 | 8 |
| ATOM | 9235 | OE2 | GLU | C | 748 | 35.360 | 61.744 | 21.503 | 1.00 | 76.18 | 8 |
| ATOM | 9236 | C | GLU | C | 748 | 35.975 | 63.585 | 19.611 | 1.00 | 22.18 | 6 |
| ATOM | 9237 | O | GLU | C | 748 | 36.345 | 62.466 | 19.273 | 1.00 | 21.12 | 8 |
| ATOM | 9238 | N | VAL | C | 749 | 36.789 | 64.462 | 20.176 | 1.00 | 36.56 | 7 |
| ATOM | 9239 | CA | VAL | C | 749 | 38.190 | 64.166 | 20.427 | 1.00 | 38.14 | 6 |
| ATOM | 9240 | CB | VAL | C | 749 | 39.072 | 65.147 | 19.673 | 1.00 | 13.87 | 6 |
| ATOM | 9241 | CG1 | VAL | C | 749 | 40.491 | 65.057 | 20.173 | 1.00 | 13.87 | 6 |
| ATOM | 9242 | CG2 | VAL | C | 749 | 38.982 | 64.870 | 18.201 | 1.00 | 13.87 | 6 |
| ATOM | 9243 | C | VAL | C | 749 | 38.608 | 64.206 | 21.894 | 1.00 | 40.54 | 6 |
| ATOM | 9244 | O | VAL | C | 749 | 38.192 | 65.072 | 22.660 | 1.00 | 41.94 | 8 |
| ATOM | 9245 | N | LYS | C | 750 | 39.456 | 63.261 | 22.271 | 1.00 | 37.16 | 7 |
| ATOM | 9246 | CA | LYS | C | 750 | 39.967 | 63.170 | 23.627 | 1.00 | 37.78 | 6 |
| ATOM | 9247 | CB | LYS | C | 750 | 39.821 | 61.736 | 24.137 | 1.00 | 72.44 | 6 |
| ATOM | 9248 | CG | LYS | C | 750 | 40.427 | 60.691 | 23.202 | 1.00 | 74.47 | 6 |
| ATOM | 9249 | CD | LYS | C | 750 | 39.798 | 59.302 | 23.376 | 1.00 | 75.51 | 6 |
| ATOM | 9250 | CE | LYS | C | 750 | 40.386 | 58.286 | 22.391 | 1.00 | 74.34 | 6 |
| ATOM | 9251 | NZ | LYS | C | 750 | 39.770 | 56.934 | 22.485 | 1.00 | 74.01 | 7 |
| ATOM | 9252 | C | LYS | C | 750 | 41.431 | 63.569 | 23.544 | 1.00 | 37.97 | 6 |
| ATOM | 9253 | O | LYS | C | 750 | 41.969 | 63.794 | 22.460 | 1.00 | 36.77 | 8 |
| ATOM | 9254 | N | PRO | C | 751 | 42.094 | 63.700 | 24.685 | 1.00 | 24.59 | 7 |
| ATOM | 9255 | CD | PRO | C | 751 | 41.687 | 63.718 | 26.100 | 1.00 | 48.62 | 6 |
| ATOM | 9256 | CA | PRO | C | 751 | 43.490 | 64.078 | 24.532 | 1.00 | 26.26 | 6 |
| ATOM | 9257 | CB | PRO | C | 751 | 43.838 | 64.642 | 25.902 | 1.00 | 48.60 | 6 |
| ATOM | 9258 | CG | PRO | C | 751 | 43.022 | 63.780 | 26.814 | 1.00 | 49.06 | 6 |
| ATOM | 9259 | C | PRO | C | 751 | 44.277 | 62.824 | 24.178 | 1.00 | 27.98 | 6 |
| ATOM | 9260 | O | PRO | C | 751 | 44.156 | 61.791 | 24.850 | 1.00 | 27.80 | 8 |
| ATOM | 9261 | N | GLY | C | 752 | 45.054 | 62.918 | 23.099 | 1.00 | 97.48 | 7 |
| ATOM | 9262 | CA | GLY | C | 752 | 45.865 | 61.800 | 22.653 | 1.00 | 96.93 | 6 |
| ATOM | 9263 | C | GLY | C | 752 | 45.563 | 61.310 | 21.247 | 1.00 | 96.17 | 6 |
| ATOM | 9264 | O | GLY | C | 752 | 46.444 | 60.777 | 20.571 | 1.00 | 96.71 | 8 |
| ATOM | 9265 | N | ASP | C | 753 | 44.322 | 61.498 | 20.801 | 1.00 | 39.63 | 7 |
| ATOM | 9266 | CA | ASP | C | 753 | 43.893 | 61.050 | 19.478 | 1.00 | 38.92 | 6 |
| ATOM | 9267 | CB | ASP | C | 753 | 42.410 | 61.355 | 19.292 | 1.00 | 59.87 | 6 |
| ATOM | 9268 | CG | ASP | C | 753 | 41.535 | 60.535 | 20.214 | 1.00 | 61.42 | 6 |
| ATOM | 9269 | OD1 | ASP | C | 753 | 40.289 | 60.633 | 20.108 | 1.00 | 61.59 | 8 |
| ATOM | 9270 | OD2 | ASP | C | 753 | 42.104 | 59.793 | 21.045 | 1.00 | 62.12 | 8 |
| ATOM | 9271 | C | ASP | C | 753 | 44.688 | 61.630 | 18.318 | 1.00 | 37.17 | 6 |
| ATOM | 9272 | O | ASP | C | 753 | 45.423 | 62.592 | 18.486 | 1.00 | 37.59 | 8 |
| ATOM | 9273 | N | ILE | C | 754 | 44.531 | 61.040 | 17.137 | 1.00 | 26.11 | 7 |
| ATOM | 9274 | CA | ILE | C | 754 | 45.260 | 61.499 | 15.961 | 1.00 | 23.46 | 6 |
| ATOM | 9275 | CB | ILE | C | 754 | 45.497 | 60.352 | 14.948 | 1.00 | 16.19 | 6 |
| ATOM | 9276 | CG2 | ILE | C | 754 | 46.279 | 60.872 | 13.756 | 1.00 | 15.99 | 6 |
| ATOM | 9277 | CG1 | ILE | C | 754 | 46.196 | 59.160 | 15.631 | 1.00 | 16.51 | 6 |
| ATOM | 9278 | CD1 | ILE | C | 754 | 47.629 | 59.389 | 16.079 | 1.00 | 14.91 | 6 |
| ATOM | 9279 | C | ILE | C | 754 | 44.516 | 62.613 | 15.237 | 1.00 | 20.74 | 6 |
| ATOM | 9280 | O | ILE | C | 754 | 43.981 | 62.411 | 14.157 | 1.00 | 20.04 | 8 |
| ATOM | 9281 | N | LEU | C | 755 | 44.496 | 63.791 | 15.846 | 1.00 | 26.14 | 7 |
| ATOM | 9282 | CA | LEU | C | 755 | 43.852 | 64.976 | 15.294 | 1.00 | 22.56 | 6 |
| ATOM | 9283 | CB | LEU | C | 755 | 44.432 | 66.181 | 16.005 | 1.00 | 20.32 | 6 |
| ATOM | 9284 | CG | LEU | C | 755 | 43.540 | 67.393 | 15.999 | 1.00 | 20.63 | 6 |
| ATOM | 9285 | CD1 | LEU | C | 755 | 43.437 | 67.869 | 14.556 | 1.00 | 22.66 | 6 |
| ATOM | 9286 | CD2 | LEU | C | 755 | 42.172 | 67.036 | 16.595 | 1.00 | 19.01 | 6 |
| ATOM | 9287 | C | LEU | C | 755 | 44.049 | 65.095 | 13.786 | 1.00 | 21.40 | 6 |
| ATOM | 9288 | O | LEU | C | 755 | 43.126 | 65.382 | 13.055 | 1.00 | 16.16 | 8 |
| ATOM | 9289 | N | VAL | C | 756 | 45.283 | 64.894 | 13.345 | 1.00 | 66.98 | 7 |
| ATOM | 9290 | CA | VAL | C | 756 | 45.666 | 64.907 | 11.926 | 1.00 | 69.54 | 6 |
| ATOM | 9291 | CB | VAL | C | 756 | 45.933 | 66.312 | 11.350 | 1.00 | 18.63 | 6 |
| ATOM | 9292 | CG1 | VAL | C | 756 | 46.505 | 66.172 | 9.947 | 1.00 | 15.76 | 6 |
| ATOM | 9293 | CG2 | VAL | C | 756 | 44.657 | 67.146 | 11.336 | 1.00 | 18.49 | 6 |
| ATOM | 9294 | C | VAL | C | 756 | 46.988 | 64.167 | 11.865 | 1.00 | 71.56 | 6 |
| ATOM | 9295 | O | VAL | C | 756 | 47.870 | 64.414 | 12.680 | 1.00 | 74.23 | 8 |
| ATOM | 9296 | N | GLY | C | 757 | 47.149 | 63.274 | 10.905 | 1.00 | 23.06 | 7 |
| ATOM | 9297 | CA | GLY | C | 757 | 48.395 | 62.544 | 10.863 | 1.00 | 24.27 | 6 |
| ATOM | 9298 | C | GLY | C | 757 | 49.137 | 62.674 | 9.566 | 1.00 | 25.32 | 6 |
| ATOM | 9299 | O | GLY | C | 757 | 48.529 | 62.731 | 8.496 | 1.00 | 26.18 | 8 |
| ATOM | 9300 | N | ARG | C | 758 | 50.460 | 62.754 | 9.660 | 1.00 | 28.46 | 7 |
| ATOM | 9301 | CA | ARG | C | 758 | 51.281 | 62.834 | 8.463 | 1.00 | 27.52 | 6 |
| ATOM | 9302 | CB | ARG | C | 758 | 51.556 | 64.302 | 8.053 | 1.00 | 31.61 | 6 |
| ATOM | 9303 | CG | ARG | C | 758 | 52.750 | 65.005 | 8.673 | 1.00 | 27.68 | 6 |
| ATOM | 9304 | CD | ARG | C | 758 | 52.462 | 65.585 | 10.051 | 1.00 | 24.98 | 6 |
| ATOM | 9305 | NE | ARG | C | 758 | 53.632 | 66.283 | 10.573 | 1.00 | 22.57 | 7 |
| ATOM | 9306 | CZ | ARG | C | 758 | 54.214 | 67.298 | 9.951 | 1.00 | 20.92 | 6 |
| ATOM | 9307 | NH1 | ARG | C | 758 | 53.731 | 67.731 | 8.793 | 1.00 | 21.14 | 7 |
| ATOM | 9308 | NH2 | ARG | C | 758 | 55.283 | 67.868 | 10.476 | 1.00 | 18.96 | 7 |
| ATOM | 9309 | C | ARG | C | 758 | 52.558 | 62.021 | 8.675 | 1.00 | 28.41 | 6 |
| ATOM | 9310 | O | ARG | C | 758 | 52.877 | 61.608 | 9.792 | 1.00 | 27.52 | 8 |
| ATOM | 9311 | N | THR | C | 759 | 53.276 | 61.774 | 7.593 | 1.00 | 38.73 | 7 |
| ATOM | 9312 | CA | THR | C | 759 | 54.470 | 60.955 | 7.657 | 1.00 | 42.64 | 6 |
| ATOM | 9313 | CB | THR | C | 759 | 54.193 | 59.641 | 6.910 | 1.00 | 66.71 | 6 |
| ATOM | 9314 | OG1 | THR | C | 759 | 53.724 | 59.924 | 5.579 | 1.00 | 70.05 | 8 |
| ATOM | 9315 | CG2 | THR | C | 759 | 53.119 | 58.841 | 7.649 | 1.00 | 66.40 | 6 |
| ATOM | 9316 | C | THR | C | 759 | 55.782 | 61.568 | 7.144 | 1.00 | 44.26 | 6 |
| ATOM | 9317 | O | THR | C | 759 | 55.884 | 62.058 | 6.016 | 1.00 | 43.54 | 8 |
| ATOM | 9318 | N | SER | C | 760 | 56.794 | 61.542 | 7.992 | 1.00 | 92.34 | 7 |
| ATOM | 9319 | CA | SER | C | 760 | 58.090 | 62.050 | 7.602 | 1.00 | 96.05 | 6 |
| ATOM | 9320 | CB | SER | C | 760 | 58.802 | 62.664 | 8.804 | 1.00 | 59.96 | 6 |
| ATOM | 9321 | OG | SER | C | 760 | 60.156 | 62.940 | 8.492 | 1.00 | 60.72 | 8 |
| ATOM | 9322 | C | SER | C | 760 | 58.876 | 60.851 | 7.079 | 1.00 | 98.27 | 6 |
| ATOM | 9323 | O | SER | C | 760 | 58.360 | 59.731 | 7.049 | 1.00 | 100.07 | 8 |
| ATOM | 9324 | N | PHE | C | 761 | 60.116 | 61.083 | 6.666 | 1.00 | 30.80 | 7 |
| ATOM | 9325 | CA | PHE | C | 761 | 60.957 | 60.008 | 6.154 | 1.00 | 32.27 | 6 |
| ATOM | 9326 | CB | PHE | C | 761 | 61.508 | 60.383 | 4.769 | 1.00 | 31.26 | 6 |
| ATOM | 9327 | CG | PHE | C | 761 | 60.546 | 60.138 | 3.634 | 1.00 | 29.23 | 6 |
| ATOM | 9328 | CD1 | PHE | C | 761 | 59.374 | 60.875 | 3.517 | 1.00 | 28.64 | 6 |
| ATOM | 9329 | CD2 | PHE | C | 761 | 60.804 | 59.155 | 2.695 | 1.00 | 27.90 | 6 |
| ATOM | 9330 | CE1 | PHE | C | 761 | 58.473 | 60.632 | 2.479 | 1.00 | 27.46 | 6 |
| ATOM | 9331 | CE2 | PHE | C | 761 | 59.909 | 58.908 | 1.660 | 1.00 | 26.39 | 6 |
| ATOM | 9332 | CZ | PHE | C | 761 | 58.741 | 59.648 | 1.553 | 1.00 | 26.21 | 6 |
| ATOM | 9333 | C | PHE | C | 761 | 62.113 | 59.749 | 7.123 | 1.00 | 34.39 | 6 |
| ATOM | 9334 | O | PHE | C | 761 | 62.434 | 58.597 | 7.443 | 1.00 | 32.45 | 8 |
| ATOM | 9335 | N | LYS | C | 762 | 62.719 | 60.846 | 7.574 | 1.00 | 75.69 | 7 |
| ATOM | 9336 | CA | LYS | C | 762 | 63.852 | 60.837 | 8.497 | 1.00 | 80.19 | 6 |
| ATOM | 9337 | CB | LYS | C | 762 | 63.389 | 61.236 | 9.902 | 1.00 | 104.57 | 6 |
| ATOM | 9338 | CG | LYS | C | 762 | 62.855 | 62.659 | 9.997 | 1.00 | 105.65 | 6 |
| ATOM | 9339 | CD | LYS | C | 762 | 62.386 | 62.983 | 11.407 | 1.00 | 106.46 | 6 |
| ATOM | 9340 | CE | LYS | C | 762 | 61.668 | 64.323 | 11.467 | 1.00 | 107.14 | 6 |
| ATOM | 9341 | NZ | LYS | C | 762 | 61.006 | 64.553 | 12.784 | 1.00 | 107.16 | 7 |
| ATOM | 9342 | C | LYS | C | 762 | 64.619 | 59.520 | 8.560 | 1.00 | 82.20 | 6 |
| ATOM | 9343 | O | LYS | C | 762 | 64.104 | 58.507 | 9.033 | 1.00 | 82.18 | 8 |
| ATOM | 9344 | N | GLY | C | 763 | 65.858 | 59.552 | 8.078 | 1.00 | 98.12 | 7 |
| ATOM | 9345 | CA | GLY | C | 763 | 66.694 | 58.368 | 8.091 | 1.00 | 100.72 | 6 |
| ATOM | 9346 | C | GLY | C | 763 | 67.747 | 58.497 | 9.169 | 1.00 | 102.72 | 6 |
| ATOM | 9347 | O | GLY | C | 763 | 68.768 | 59.149 | 8.966 | 1.00 | 102.66 | 8 |
| ATOM | 9348 | N | GLU | C | 764 | 67.496 | 57.875 | 10.317 | 1.00 | 155.30 | 7 |
| ATOM | 9349 | CA | GLU | C | 764 | 68.412 | 57.927 | 11.453 | 1.00 | 157.88 | 6 |
| ATOM | 9350 | CB | GLU | C | 764 | 67.652 | 57.620 | 12.746 | 1.00 | 99.28 | 6 |
| ATOM | 9351 | CG | GLU | C | 764 | 66.577 | 58.642 | 13.111 | 1.00 | 98.82 | 6 |
| ATOM | 9352 | CD | GLU | C | 764 | 65.591 | 58.895 | 11.987 | 1.00 | 98.23 | 6 |
| ATOM | 9353 | OE1 | GLU | C | 764 | 65.826 | 59.815 | 11.173 | 1.00 | 97.44 | 8 |
| ATOM | 9354 | OE2 | GLU | C | 764 | 64.587 | 58.160 | 11.913 | 1.00 | 97.72 | 8 |
| ATOM | 9355 | C | GLU | C | 764 | 69.590 | 56.965 | 11.316 | 1.00 | 159.07 | 6 |
| ATOM | 9356 | O | GLU | C | 764 | 70.739 | 57.339 | 11.558 | 1.00 | 159.14 | 8 |
| ATOM | 9357 | N | GLN | C | 765 | 69.297 | 55.726 | 10.935 | 1.00 | 78.66 | 7 |
| ATOM | 9358 | CA | GLN | C | 765 | 70.331 | 54.709 | 10.774 | 1.00 | 79.59 | 6 |
| ATOM | 9359 | CB | GLN | C | 765 | 70.469 | 53.906 | 12.070 | 1.00 | 174.48 | 6 |
| ATOM | 9360 | CG | GLN | C | 765 | 70.760 | 54.750 | 13.303 | 1.00 | 175.53 | 6 |
| ATOM | 9361 | CD | GLN | C | 765 | 70.693 | 53.951 | 14.593 | 1.00 | 176.86 | 6 |
| ATOM | 9362 | OE1 | GLN | C | 765 | 71.366 | 52.931 | 14.740 | 1.00 | 176.70 | 8 |
| ATOM | 9363 | NE2 | GLN | C | 765 | 69.879 | 54.416 | 15.537 | 1.00 | 177.18 | 7 |
| ATOM | 9364 | C | GLN | C | 765 | 69.999 | 53.752 | 9.629 | 1.00 | 79.76 | 6 |
| ATOM | 9365 | O | GLN | C | 765 | 68.836 | 53.594 | 9.254 | 1.00 | 79.42 | 8 |
| ATOM | 9366 | N | GLU | C | 766 | 71.027 | 53.123 | 9.072 | 1.00 | 83.53 | 7 |
| ATOM | 9367 | CA | GLU | C | 766 | 70.842 | 52.155 | 7.996 | 1.00 | 84.28 | 6 |
| ATOM | 9368 | CB | GLU | C | 766 | 71.962 | 52.291 | 6.963 | 1.00 | 112.35 | 6 |
| ATOM | 9369 | CG | GLU | C | 766 | 71.891 | 53.564 | 6.134 | 1.00 | 114.45 | 6 |
| ATOM | 9370 | CD | GLU | C | 766 | 70.773 | 53.524 | 5.105 | 1.00 | 115.73 | 6 |
| ATOM | 9371 | OE1 | GLU | C | 766 | 70.506 | 54.562 | 4.457 | 1.00 | 116.02 | 8 |
| ATOM | 9372 | OE2 | GLU | C | 766 | 70.166 | 52.446 | 4.938 | 1.00 | 116.55 | 8 |
| ATOM | 9373 | C | GLU | C | 766 | 70.891 | 50.774 | 8.649 | 1.00 | 84.41 | 6 |
| ATOM | 9374 | O | GLU | C | 766 | 71.949 | 50.337 | 9.101 | 1.00 | 84.45 | 8 |
| ATOM | 9375 | N | PRO | C | 767 | 69.746 | 50.069 | 8.710 | 1.00 | 84.90 | 7 |
| ATOM | 9376 | CD | PRO | C | 767 | 68.476 | 50.357 | 8.019 | 1.00 | 102.93 | 6 |
| ATOM | 9377 | CA | PRO | C | 767 | 69.696 | 48.740 | 9.325 | 1.00 | 85.00 | 6 |
| ATOM | 9378 | CB | PRO | C | 767 | 68.218 | 48.371 | 9.223 | 1.00 | 102.04 | 6 |
| ATOM | 9379 | CG | PRO | C | 767 | 67.835 | 48.974 | 7.919 | 1.00 | 102.18 | 6 |
| ATOM | 9380 | C | PRO | C | 767 | 70.585 | 47.725 | 8.625 | 1.00 | 85.38 | 6 |
| ATOM | 9381 | O | PRO | C | 767 | 70.357 | 47.389 | 7.461 | 1.00 | 85.98 | 8 |
| ATOM | 9382 | N | SER | C | 768 | 71.600 | 47.242 | 9.335 | 1.00 | 120.88 | 7 |
| ATOM | 9383 | CA | SER | C | 768 | 72.503 | 46.245 | 8.777 | 1.00 | 121.38 | 6 |
| ATOM | 9384 | CB | SER | C | 768 | 73.617 | 45.907 | 9.765 | 1.00 | 93.71 | 6 |
| ATOM | 9385 | OG | SER | C | 768 | 73.150 | 44.975 | 10.726 | 1.00 | 93.67 | 8 |
| ATOM | 9386 | C | SER | C | 768 | 71.630 | 45.024 | 8.602 | 1.00 | 121.74 | 6 |
| ATOM | 9387 | O | SER | C | 768 | 70.459 | 45.044 | 8.973 | 1.00 | 121.95 | 8 |
| ATOM | 9388 | N | PRO | C | 769 | 72.179 | 43.938 | 8.045 | 1.00 | 102.93 | 7 |
| ATOM | 9389 | CD | PRO | C | 769 | 73.580 | 43.580 | 7.766 | 1.00 | 89.35 | 6 |
| ATOM | 9390 | CA | PRO | C | 769 | 71.308 | 42.773 | 7.898 | 1.00 | 103.50 | 6 |
| ATOM | 9391 | CB | PRO | C | 769 | 72.210 | 41.762 | 7.207 | 1.00 | 90.02 | 6 |
| ATOM | 9392 | CG | PRO | C | 769 | 73.538 | 42.063 | 7.836 | 1.00 | 90.05 | 6 |
| ATOM | 9393 | C | PRO | C | 769 | 70.904 | 42.325 | 9.298 | 1.00 | 104.29 | 6 |
| ATOM | 9394 | O | PRO | C | 769 | 70.813 | 43.147 | 10.216 | 1.00 | 104.46 | 8 |
| ATOM | 9395 | N | GLU | C | 770 | 70.690 | 41.027 | 9.471 | 1.00 | 155.25 | 7 |
| ATOM | 9396 | CA | GLU | C | 770 | 70.296 | 40.510 | 10.772 | 1.00 | 155.10 | 6 |
| ATOM | 9397 | CB | GLU | C | 770 | 71.214 | 41.065 | 11.867 | 1.00 | 64.96 | 6 |
| ATOM | 9398 | CG | GLU | C | 770 | 72.702 | 41.012 | 11.532 | 1.00 | 63.46 | 6 |
| ATOM | 9399 | CD | GLU | C | 770 | 73.562 | 41.751 | 12.540 | 1.00 | 61.50 | 6 |
| ATOM | 9400 | OE1 | GLU | C | 770 | 73.340 | 42.962 | 12.737 | 1.00 | 60.08 | 8 |
| ATOM | 9401 | OE2 | GLU | C | 770 | 74.463 | 41.125 | 13.136 | 1.00 | 61.22 | 8 |
| ATOM | 9402 | C | GLU | C | 770 | 68.872 | 40.986 | 11.008 | 1.00 | 155.66 | 6 |
| ATOM | 9403 | O | GLU | C | 770 | 67.985 | 40.196 | 11.325 | 1.00 | 156.68 | 8 |
| ATOM | 9404 | N | GLU | C | 771 | 68.662 | 42.290 | 10.851 | 1.00 | 47.62 | 7 |
| ATOM | 9405 | CA | GLU | C | 771 | 67.345 | 42.874 | 11.026 | 1.00 | 47.75 | 6 |
| ATOM | 9406 | CB | GLU | C | 771 | 67.452 | 44.363 | 11.354 | 1.00 | 183.49 | 6 |
| ATOM | 9407 | CG | GLU | C | 771 | 67.730 | 44.639 | 12.822 | 1.00 | 184.01 | 6 |
| ATOM | 9408 | CD | GLU | C | 771 | 67.770 | 46.118 | 13.139 | 1.00 | 184.75 | 6 |
| ATOM | 9409 | OE1 | GLU | C | 771 | 66.836 | 46.841 | 12.733 | 1.00 | 185.21 | 8 |
| ATOM | 9410 | OE2 | GLU | C | 771 | 68.732 | 46.559 | 13.801 | 1.00 | 185.18 | 8 |
| ATOM | 9411 | C | GLU | C | 771 | 66.548 | 42.670 | 9.752 | 1.00 | 47.79 | 6 |
| ATOM | 9412 | O | GLU | C | 771 | 65.551 | 41.960 | 9.757 | 1.00 | 46.93 | 8 |
| ATOM | 9413 | N | ARG | C | 772 | 66.989 | 43.275 | 8.655 | 1.00 | 97.85 | 7 |
| ATOM | 9414 | CA | ARG | C | 772 | 66.276 | 43.119 | 7.393 | 1.00 | 99.27 | 6 |
| ATOM | 9415 | CB | ARG | C | 772 | 67.123 | 43.650 | 6.232 | 1.00 | 195.76 | 6 |
| ATOM | 9416 | CG | ARG | C | 772 | 66.331 | 44.148 | 5.011 | 1.00 | 198.00 | 6 |
| ATOM | 9417 | CD | ARG | C | 772 | 65.582 | 43.039 | 4.275 | 1.00 | 198.91 | 6 |
| ATOM | 9418 | NE | ARG | C | 772 | 65.100 | 43.490 | 2.970 | 1.00 | 198.07 | 7 |
| ATOM | 9419 | CZ | ARG | C | 772 | 64.426 | 42.726 | 2.116 | 1.00 | 197.88 | 6 |
| ATOM | 9420 | NH1 | ARG | C | 772 | 64.148 | 41.468 | 2.429 | 1.00 | 198.35 | 7 |
| ATOM | 9421 | NH2 | ARG | C | 772 | 64.040 | 43.214 | 0.945 | 1.00 | 197.11 | 7 |
| ATOM | 9422 | C | ARG | C | 772 | 65.996 | 41.629 | 7.212 | 1.00 | 98.44 | 6 |
| ATOM | 9423 | O | ARG | C | 772 | 65.008 | 41.244 | 6.586 | 1.00 | 97.98 | 8 |
| ATOM | 9424 | N | LEU | C | 773 | 66.873 | 40.796 | 7.770 | 1.00 | 84.36 | 7 |
| ATOM | 9425 | CA | LEU | C | 773 | 66.703 | 39.353 | 7.685 | 1.00 | 83.03 | 6 |
| ATOM | 9426 | CB | LEU | C | 773 | 67.942 | 38.613 | 8.189 | 1.00 | 67.30 | 6 |
| ATOM | 9427 | CG | LEU | C | 773 | 67.775 | 37.087 | 8.242 | 1.00 | 66.86 | 6 |
| ATOM | 9428 | CD1 | LEU | C | 773 | 67.151 | 36.597 | 6.940 | 1.00 | 66.68 | 6 |
| ATOM | 9429 | CD2 | LEU | C | 773 | 69.126 | 36.418 | 8.494 | 1.00 | 67.40 | 6 |
| ATOM | 9430 | C | LEU | C | 773 | 65.517 | 38.983 | 8.543 | 1.00 | 82.47 | 6 |
| ATOM | 9431 | O | LEU | C | 773 | 64.527 | 38.458 | 8.046 | 1.00 | 82.83 | 8 |
| ATOM | 9432 | N | LEU | C | 774 | 65.616 | 39.240 | 9.841 | 1.00 | 127.21 | 7 |
| ATOM | 9433 | CA | LEU | C | 774 | 64.493 | 38.937 | 10.705 | 1.00 | 126.02 | 6 |
| ATOM | 9434 | CB | LEU | C | 774 | 64.682 | 39.538 | 12.100 | 1.00 | 90.56 | 6 |
| ATOM | 9435 | CG | LEU | C | 774 | 65.899 | 39.062 | 12.892 | 1.00 | 90.26 | 6 |
| ATOM | 9436 | CD1 | LEU | C | 774 | 65.866 | 39.621 | 14.300 | 1.00 | 90.04 | 6 |
| ATOM | 9437 | CD2 | LEU | C | 774 | 65.901 | 37.557 | 12.932 | 1.00 | 89.38 | 6 |
| ATOM | 9438 | C | LEU | C | 774 | 63.335 | 39.617 | 9.999 | 1.00 | 125.72 | 6 |
| ATOM | 9439 | O | LEU | C | 774 | 62.316 | 38.988 | 9.712 | 1.00 | 127.26 | 8 |
| ATOM | 9440 | N | ALA | C | 775 | 63.530 | 40.899 | 9.686 | 1.00 | 31.75 | 7 |
| ATOM | 9441 | CA | ALA | C | 775 | 62.523 | 41.699 | 9.000 | 1.00 | 30.14 | 6 |
| ATOM | 9442 | CB | ALA | C | 775 | 63.152 | 42.977 | 8.445 | 1.00 | 97.68 | 6 |
| ATOM | 9443 | C | ALA | C | 775 | 61.918 | 40.862 | 7.875 | 1.00 | 29.04 | 6 |
| ATOM | 9444 | O | ALA | C | 775 | 60.707 | 40.584 | 7.846 | 1.00 | 27.53 | 8 |
| ATOM | 9445 | N | SER | C | 776 | 62.775 | 40.443 | 6.954 | 1.00 | 69.63 | 7 |
| ATOM | 9446 | CA | SER | C | 776 | 62.313 | 39.626 | 5.857 | 1.00 | 70.01 | 6 |
| ATOM | 9447 | CB | SER | C | 776 | 63.491 | 39.198 | 4.970 | 1.00 | 208.87 | 6 |
| ATOM | 9448 | OG | SER | C | 776 | 64.495 | 38.524 | 5.708 | 1.00 | 208.87 | 8 |
| ATOM | 9449 | C | SER | C | 776 | 61.636 | 38.419 | 6.478 | 1.00 | 68.77 | 6 |
| ATOM | 9450 | O | SER | C | 776 | 60.417 | 38.273 | 6.397 | 1.00 | 68.62 | 8 |
| ATOM | 9451 | N | ILE | C | 777 | 62.437 | 37.585 | 7.134 | 1.00 | 54.34 | 7 |
| ATOM | 9452 | CA | ILE | C | 777 | 61.944 | 36.376 | 7.766 | 1.00 | 54.13 | 6 |
| ATOM | 9453 | CB | ILE | C | 777 | 62.918 | 35.857 | 8.808 | 1.00 | 88.03 | 6 |
| ATOM | 9454 | CG2 | ILE | C | 777 | 62.315 | 34.659 | 9.513 | 1.00 | 87.22 | 6 |
| ATOM | 9455 | CG1 | ILE | C | 777 | 64.228 | 35.474 | 8.124 | 1.00 | 89.30 | 6 |
| ATOM | 9456 | CD1 | ILE | C | 777 | 65.262 | 34.896 | 9.054 | 1.00 | 91.23 | 6 |
| ATOM | 9457 | C | ILE | C | 777 | 60.602 | 36.587 | 8.424 | 1.00 | 53.63 | 6 |
| ATOM | 9458 | O | ILE | C | 777 | 59.571 | 36.209 | 7.874 | 1.00 | 54.38 | 8 |
| ATOM | 9459 | N | PHE | C | 778 | 60.612 | 37.191 | 9.600 | 1.00 | 80.03 | 7 |
| ATOM | 9460 | CA | PHE | C | 778 | 59.379 | 37.447 | 10.317 | 1.00 | 80.08 | 6 |
| ATOM | 9461 | CB | PHE | C | 778 | 59.698 | 37.795 | 11.767 | 1.00 | 79.43 | 6 |
| ATOM | 9462 | CG | PHE | C | 778 | 60.573 | 36.779 | 12.428 | 1.00 | 79.06 | 6 |
| ATOM | 9463 | CD1 | PHE | C | 778 | 61.896 | 36.612 | 12.026 | 1.00 | 78.85 | 6 |
| ATOM | 9464 | CD2 | PHE | C | 778 | 60.059 | 35.923 | 13.386 | 1.00 | 78.93 | 6 |
| ATOM | 9465 | CE1 | PHE | C | 778 | 62.682 | 35.605 | 12.562 | 1.00 | 78.25 | 6 |
| ATOM | 9466 | CE2 | PHE | C | 778 | 60.844 | 34.912 | 13.927 | 1.00 | 78.31 | 6 |
| ATOM | 9467 | CZ | PHE | C | 778 | 62.153 | 34.753 | 13.511 | 1.00 | 77.66 | 6 |
| ATOM | 9468 | C | PHE | C | 778 | 58.620 | 38.571 | 9.637 | 1.00 | 81.48 | 6 |
| ATOM | 9469 | O | PHE | C | 778 | 58.211 | 39.530 | 10.287 | 1.00 | 82.69 | 8 |
| ATOM | 9470 | N | GLY | C | 779 | 58.451 | 38.428 | 8.319 | 1.00 | 83.84 | 7 |
| ATOM | 9471 | CA | GLY | C | 779 | 57.738 | 39.394 | 7.496 | 1.00 | 85.02 | 6 |
| ATOM | 9472 | C | GLY | C | 779 | 57.593 | 40.754 | 8.133 | 1.00 | 86.05 | 6 |
| ATOM | 9473 | O | GLY | C | 779 | 56.496 | 41.308 | 8.233 | 1.00 | 86.31 | 8 |
| ATOM | 9474 | N | ALA | C | 780 | 58.721 | 41.295 | 8.563 | 1.00 | 58.08 | 7 |
| ATOM | 9475 | CA | ALA | C | 780 | 58.741 | 42.580 | 9.218 | 1.00 | 59.13 | 6 |
| ATOM | 9476 | CB | ALA | C | 780 | 59.834 | 42.595 | 10.254 | 1.00 | 88.73 | 6 |
| ATOM | 9477 | C | ALA | C | 780 | 58.934 | 43.708 | 8.224 | 1.00 | 60.39 | 6 |
| ATOM | 9478 | O | ALA | C | 780 | 59.800 | 44.565 | 8.403 | 1.00 | 61.00 | 8 |
| ATOM | 9479 | N | ALA | C | 781 | 58.114 | 43.694 | 7.177 | 1.00 | 105.56 | 7 |
| ATOM | 9480 | CA | ALA | C | 781 | 58.147 | 44.706 | 6.126 | 1.00 | 106.36 | 6 |
| ATOM | 9481 | CB | ALA | C | 781 | 56.728 | 45.016 | 5.670 | 1.00 | 152.25 | 6 |
| ATOM | 9482 | C | ALA | C | 781 | 58.829 | 45.983 | 6.597 | 1.00 | 106.65 | 6 |
| ATOM | 9483 | O | ALA | C | 781 | 58.381 | 46.615 | 7.554 | 1.00 | 106.54 | 8 |
| ATOM | 9484 | N | ALA | C | 782 | 59.913 | 46.354 | 5.922 | 1.00 | 90.59 | 7 |
| ATOM | 9485 | CA | ALA | C | 782 | 60.665 | 47.552 | 6.276 | 1.00 | 91.46 | 6 |
| ATOM | 9486 | CB | ALA | C | 782 | 61.874 | 47.695 | 5.358 | 1.00 | 46.35 | 6 |
| ATOM | 9487 | C | ALA | C | 782 | 59.811 | 48.822 | 6.231 | 1.00 | 92.22 | 6 |
| ATOM | 9488 | O | ALA | C | 782 | 58.659 | 48.805 | 5.783 | 1.00 | 92.80 | 8 |
| ATOM | 9489 | N | ARG | C | 783 | 60.390 | 49.925 | 6.695 | 1.00 | 127.32 | 7 |
| ATOM | 9490 | CA | ARG | C | 783 | 59.688 | 51.201 | 6.735 | 1.00 | 127.42 | 6 |
| ATOM | 9491 | CB | ARG | C | 783 | 59.027 | 51.374 | 8.103 | 1.00 | 132.46 | 6 |
| ATOM | 9492 | CG | ARG | C | 783 | 60.036 | 51.446 | 9.247 | 1.00 | 133.87 | 6 |
| ATOM | 9493 | CD | ARG | C | 783 | 59.374 | 51.400 | 10.617 | 1.00 | 134.71 | 6 |
| ATOM | 9494 | NE | ARG | C | 783 | 60.363 | 51.483 | 11.688 | 1.00 | 135.25 | 7 |
| ATOM | 9495 | CZ | ARG | C | 783 | 60.092 | 51.290 | 12.974 | 1.00 | 135.83 | 6 |
| ATOM | 9496 | NH1 | ARG | C | 783 | 58.860 | 51.001 | 13.358 | 1.00 | 135.68 | 7 |
| ATOM | 9497 | NH2 | ARG | C | 783 | 61.055 | 51.387 | 13.879 | 1.00 | 136.98 | 7 |
| ATOM | 9498 | C | ARG | C | 783 | 60.638 | 52.370 | 6.487 | 1.00 | 126.33 | 6 |
| ATOM | 9499 | O | ARG | C | 783 | 61.831 | 52.280 | 6.768 | 1.00 | 125.68 | 8 |
| ATOM | 9500 | N | ASP | C | 784 | 60.094 | 53.462 | 5.961 | 1.00 | 96.81 | 7 |
| ATOM | 9501 | CA | ASP | C | 784 | 60.858 | 54.677 | 5.686 | 1.00 | 96.62 | 6 |
| ATOM | 9502 | CB | ASP | C | 784 | 61.078 | 54.858 | 4.179 | 1.00 | 165.71 | 6 |
| ATOM | 9503 | CG | ASP | C | 784 | 62.224 | 54.027 | 3.642 | 1.00 | 167.54 | 6 |
| ATOM | 9504 | OD1 | ASP | C | 784 | 63.387 | 54.298 | 4.011 | 1.00 | 168.36 | 8 |
| ATOM | 9505 | OD2 | ASP | C | 784 | 61.961 | 53.106 | 2.843 | 1.00 | 168.28 | 8 |
| ATOM | 9506 | C | ASP | C | 784 | 60.048 | 55.864 | 6.198 | 1.00 | 95.97 | 6 |
| ATOM | 9507 | O | ASP | C | 784 | 60.163 | 56.973 | 5.671 | 1.00 | 96.30 | 8 |
| ATOM | 9508 | N | VAL | C | 785 | 59.242 | 55.640 | 7.233 | 1.00 | 84.48 | 7 |
| ATOM | 9509 | CA | VAL | C | 785 | 58.395 | 56.707 | 7.740 | 1.00 | 81.77 | 6 |
| ATOM | 9510 | CB | VAL | C | 785 | 57.065 | 56.674 | 6.992 | 1.00 | 76.38 | 6 |
| ATOM | 9511 | CG1 | VAL | C | 785 | 55.991 | 57.305 | 7.824 | 1.00 | 78.53 | 6 |
| ATOM | 9512 | CG2 | VAL | C | 785 | 57.208 | 57.395 | 5.674 | 1.00 | 75.81 | 6 |
| ATOM | 9513 | C | VAL | C | 785 | 58.108 | 56.759 | 9.242 | 1.00 | 80.52 | 6 |
| ATOM | 9514 | O | VAL | C | 785 | 57.859 | 55.729 | 9.880 | 1.00 | 80.39 | 8 |
| ATOM | 9515 | N | LYS | C | 786 | 58.127 | 57.983 | 9.780 | 1.00 | 65.91 | 7 |
| ATOM | 9516 | CA | LYS | C | 786 | 57.851 | 58.248 | 11.196 | 1.00 | 64.98 | 6 |
| ATOM | 9517 | CB | LYS | C | 786 | 58.651 | 59.457 | 11.694 | 1.00 | 87.58 | 6 |
| ATOM | 9518 | CG | LYS | C | 786 | 60.119 | 59.203 | 11.974 | 1.00 | 88.11 | 6 |
| ATOM | 9519 | CD | LYS | C | 786 | 60.747 | 60.411 | 12.669 | 1.00 | 89.06 | 6 |
| ATOM | 9520 | CE | LYS | C | 786 | 62.174 | 60.132 | 13.132 | 1.00 | 89.59 | 6 |
| ATOM | 9521 | NZ | LYS | C | 786 | 62.786 | 61.311 | 13.811 | 1.00 | 90.44 | 7 |
| ATOM | 9522 | C | LYS | C | 786 | 56.364 | 58.542 | 11.394 | 1.00 | 64.50 | 6 |
| ATOM | 9523 | O | LYS | C | 786 | 55.696 | 59.045 | 10.485 | 1.00 | 64.82 | 8 |
| ATOM | 9524 | N | ASP | C | 787 | 55.859 | 58.258 | 12.592 | 1.00 | 78.90 | 7 |
| ATOM | 9525 | CA | ASP | C | 787 | 54.447 | 58.472 | 12.904 | 1.00 | 77.08 | 6 |
| ATOM | 9526 | CB | ASP | C | 787 | 54.040 | 57.595 | 14.095 | 1.00 | 91.21 | 6 |
| ATOM | 9527 | CG | ASP | C | 787 | 52.530 | 57.548 | 14.312 | 1.00 | 90.98 | 6 |
| ATOM | 9528 | OD1 | ASP | C | 787 | 51.926 | 58.575 | 14.689 | 1.00 | 90.77 | 8 |
| ATOM | 9529 | OD2 | ASP | C | 787 | 51.942 | 56.467 | 14.105 | 1.00 | 91.64 | 8 |
| ATOM | 9530 | C | ASP | C | 787 | 54.098 | 59.924 | 13.206 | 1.00 | 76.50 | 6 |
| ATOM | 9531 | O | ASP | C | 787 | 53.005 | 60.204 | 13.686 | 1.00 | 77.65 | 8 |
| ATOM | 9532 | N | THR | C | 788 | 55.012 | 60.847 | 12.927 | 1.00 | 93.42 | 7 |
| ATOM | 9533 | CA | THR | C | 788 | 54.758 | 62.264 | 13.191 | 1.00 | 91.67 | 6 |
| ATOM | 9534 | CB | THR | C | 788 | 55.735 | 63.169 | 12.432 | 1.00 | 95.91 | 6 |
| ATOM | 9535 | OG1 | THR | C | 788 | 55.235 | 64.511 | 12.437 | 1.00 | 96.03 | 8 |
| ATOM | 9536 | CG2 | THR | C | 788 | 55.890 | 62.704 | 10.994 | 1.00 | 96.48 | 6 |
| ATOM | 9537 | C | THR | C | 788 | 53.349 | 62.692 | 12.798 | 1.00 | 91.65 | 6 |
| ATOM | 9538 | O | THR | C | 788 | 53.109 | 63.059 | 11.651 | 1.00 | 92.72 | 8 |
| ATOM | 9539 | N | SER | C | 789 | 52.422 | 62.653 | 13.753 | 1.00 | 56.46 | 7 |
| ATOM | 9540 | CA | SER | C | 789 | 51.044 | 63.045 | 13.489 | 1.00 | 54.25 | 6 |
| ATOM | 9541 | CB | SER | C | 789 | 50.082 | 62.041 | 14.120 | 1.00 | 115.65 | 6 |
| ATOM | 9542 | OG | SER | C | 789 | 50.305 | 60.739 | 13.613 | 1.00 | 119.05 | 8 |
| ATOM | 9543 | C | SER | C | 789 | 50.805 | 64.429 | 14.074 | 1.00 | 52.20 | 6 |
| ATOM | 9544 | O | SER | C | 789 | 51.716 | 65.255 | 14.114 | 1.00 | 51.55 | 8 |
| ATOM | 9545 | N | LEU | C | 790 | 49.575 | 64.683 | 14.510 | 1.00 | 68.48 | 7 |
| ATOM | 9546 | CA | LEU | C | 790 | 49.218 | 65.962 | 15.107 | 1.00 | 66.87 | 6 |
| ATOM | 9547 | CB | LEU | C | 790 | 48.061 | 66.615 | 14.381 | 1.00 | 35.44 | 6 |
| ATOM | 9548 | CG | LEU | C | 790 | 47.605 | 67.806 | 15.206 | 1.00 | 34.54 | 6 |
| ATOM | 9549 | CD1 | LEU | C | 790 | 48.772 | 68.750 | 15.359 | 1.00 | 33.71 | 6 |
| ATOM | 9550 | CD2 | LEU | C | 790 | 46.421 | 68.493 | 14.570 | 1.00 | 34.41 | 6 |
| ATOM | 9551 | C | LEU | C | 790 | 48.801 | 65.764 | 16.543 | 1.00 | 67.04 | 6 |
| ATOM | 9552 | O | LEU | C | 790 | 49.280 | 66.458 | 17.438 | 1.00 | 67.36 | 8 |
| ATOM | 9553 | N | ARG | C | 791 | 47.880 | 64.824 | 16.737 | 1.00 | 86.02 | 7 |
| ATOM | 9554 | CA | ARG | C | 791 | 47.346 | 64.460 | 18.049 | 1.00 | 84.48 | 6 |
| ATOM | 9555 | CB | ARG | C | 791 | 48.308 | 63.509 | 18.758 | 1.00 | 62.56 | 6 |
| ATOM | 9556 | CG | ARG | C | 791 | 48.452 | 62.201 | 18.048 | 1.00 | 63.59 | 6 |
| ATOM | 9557 | CD | ARG | C | 791 | 49.277 | 61.219 | 18.835 | 1.00 | 65.39 | 6 |
| ATOM | 9558 | NE | ARG | C | 791 | 49.596 | 60.070 | 17.997 | 1.00 | 67.02 | 7 |
| ATOM | 9559 | CZ | ARG | C | 791 | 50.304 | 59.025 | 18.395 | 1.00 | 68.43 | 6 |
| ATOM | 9560 | NH1 | ARG | C | 791 | 50.773 | 58.972 | 19.633 | 1.00 | 69.44 | 7 |
| ATOM | 9561 | NH2 | ARG | C | 791 | 50.552 | 58.044 | 17.546 | 1.00 | 69.24 | 7 |
| ATOM | 9562 | C | ARG | C | 791 | 46.983 | 65.597 | 18.991 | 1.00 | 82.74 | 6 |
| ATOM | 9563 | O | ARG | C | 791 | 47.699 | 66.590 | 19.105 | 1.00 | 84.11 | 8 |
| ATOM | 9564 | N | VAL | C | 792 | 45.865 | 65.437 | 19.683 | 1.00 | 13.87 | 7 |
| ATOM | 9565 | CA | VAL | C | 792 | 45.439 | 66.461 | 20.602 | 1.00 | 13.87 | 6 |
| ATOM | 9566 | CB | VAL | C | 792 | 43.941 | 66.366 | 20.889 | 1.00 | 38.63 | 6 |
| ATOM | 9567 | CG1 | VAL | C | 792 | 43.550 | 67.385 | 21.922 | 1.00 | 39.04 | 6 |
| ATOM | 9568 | CG2 | VAL | C | 792 | 43.178 | 66.652 | 19.617 | 1.00 | 38.69 | 6 |
| ATOM | 9569 | C | VAL | C | 792 | 46.238 | 66.392 | 21.889 | 1.00 | 13.87 | 6 |
| ATOM | 9570 | O | VAL | C | 792 | 46.291 | 65.360 | 22.550 | 1.00 | 13.87 | 8 |
| ATOM | 9571 | N | PRO | C | 793 | 46.883 | 67.512 | 22.246 | 1.00 | 54.22 | 7 |
| ATOM | 9572 | CD | PRO | C | 793 | 46.682 | 68.792 | 21.547 | 1.00 | 30.05 | 6 |
| ATOM | 9573 | CA | PRO | C | 793 | 47.720 | 67.718 | 23.425 | 1.00 | 55.90 | 6 |
| ATOM | 9574 | CB | PRO | C | 793 | 48.136 | 69.176 | 23.294 | 1.00 | 31.06 | 6 |
| ATOM | 9575 | CG | PRO | C | 793 | 46.986 | 69.784 | 22.623 | 1.00 | 29.13 | 6 |
| ATOM | 9576 | C | PRO | C | 793 | 46.960 | 67.457 | 24.694 | 1.00 | 58.23 | 6 |
| ATOM | 9577 | O | PRO | C | 793 | 45.821 | 67.016 | 24.658 | 1.00 | 60.69 | 8 |
| ATOM | 9578 | N | PRO | C | 794 | 47.585 | 67.717 | 25.842 | 1.00 | 54.15 | 7 |
| ATOM | 9579 | CD | PRO | C | 794 | 49.024 | 67.961 | 26.043 | 1.00 | 99.87 | 6 |
| ATOM | 9580 | CA | PRO | C | 794 | 46.913 | 67.493 | 27.121 | 1.00 | 54.81 | 6 |
| ATOM | 9581 | CB | PRO | C | 794 | 47.929 | 68.014 | 28.123 | 1.00 | 101.03 | 6 |
| ATOM | 9582 | CG | PRO | C | 794 | 49.220 | 67.584 | 27.492 | 1.00 | 99.96 | 6 |
| ATOM | 9583 | C | PRO | C | 794 | 45.539 | 68.146 | 27.274 | 1.00 | 55.50 | 6 |
| ATOM | 9584 | O | PRO | C | 794 | 44.783 | 67.769 | 28.161 | 1.00 | 56.42 | 8 |
| ATOM | 9585 | N | GLY | C | 795 | 45.214 | 69.116 | 26.424 | 1.00 | 89.83 | 7 |
| ATOM | 9586 | CA | GLY | C | 795 | 43.911 | 69.759 | 26.515 | 1.00 | 90.80 | 6 |
| ATOM | 9587 | C | GLY | C | 795 | 42.792 | 68.770 | 26.223 | 1.00 | 91.15 | 6 |
| ATOM | 9588 | O | GLY | C | 795 | 42.706 | 68.252 | 25.115 | 1.00 | 90.96 | 8 |
| ATOM | 9589 | N | GLU | C | 796 | 41.933 | 68.522 | 27.213 | 1.00 | 162.13 | 7 |
| ATOM | 9590 | CA | GLU | C | 796 | 40.812 | 67.573 | 27.114 | 1.00 | 160.60 | 6 |
| ATOM | 9591 | CB | GLU | C | 796 | 39.703 | 67.977 | 28.102 | 1.00 | 68.33 | 6 |
| ATOM | 9592 | CG | GLU | C | 796 | 38.591 | 66.940 | 28.304 | 1.00 | 68.24 | 6 |
| ATOM | 9593 | CD | GLU | C | 796 | 37.365 | 67.177 | 27.435 | 1.00 | 69.47 | 6 |
| ATOM | 9594 | OE1 | GLU | C | 796 | 36.639 | 68.173 | 27.662 | 1.00 | 69.13 | 8 |
| ATOM | 9595 | OE2 | GLU | C | 796 | 37.123 | 66.359 | 26.523 | 1.00 | 72.01 | 8 |
| ATOM | 9596 | C | GLU | C | 796 | 40.192 | 67.320 | 25.732 | 1.00 | 159.42 | 6 |
| ATOM | 9597 | O | GLU | C | 796 | 39.453 | 66.353 | 25.559 | 1.00 | 159.93 | 8 |
| ATOM | 9598 | N | GLY | C | 797 | 40.481 | 68.174 | 24.755 | 1.00 | 54.40 | 7 |
| ATOM | 9599 | CA | GLY | C | 797 | 39.932 | 67.974 | 23.429 | 1.00 | 51.86 | 6 |
| ATOM | 9600 | C | GLY | C | 797 | 38.481 | 68.379 | 23.354 | 1.00 | 51.20 | 6 |
| ATOM | 9601 | O | GLY | C | 797 | 38.108 | 69.381 | 23.935 | 1.00 | 53.71 | 8 |
| ATOM | 9602 | N | GLY | C | 798 | 37.664 | 67.606 | 22.646 | 1.00 | 30.90 | 7 |
| ATOM | 9603 | CA | GLY | C | 798 | 36.249 | 67.919 | 22.510 | 1.00 | 26.75 | 6 |
| ATOM | 9604 | C | GLY | C | 798 | 35.797 | 67.791 | 21.063 | 1.00 | 24.13 | 6 |
| ATOM | 9605 | O | GLY | C | 798 | 36.615 | 67.829 | 20.148 | 1.00 | 24.00 | 8 |
| ATOM | 9606 | N | ILE | C | 799 | 34.496 | 67.640 | 20.849 | 1.00 | 52.65 | 7 |
| ATOM | 9607 | CA | ILE | C | 799 | 33.932 | 67.500 | 19.508 | 1.00 | 51.38 | 6 |
| ATOM | 9608 | CB | ILE | C | 799 | 32.432 | 67.843 | 19.510 | 1.00 | 37.82 | 6 |
| ATOM | 9609 | CG2 | ILE | C | 799 | 31.860 | 67.640 | 18.135 | 1.00 | 38.21 | 6 |
| ATOM | 9610 | CG1 | ILE | C | 799 | 31.685 | 66.932 | 20.482 | 1.00 | 37.62 | 6 |
| ATOM | 9611 | CD1 | ILE | C | 799 | 30.228 | 67.298 | 20.656 | 1.00 | 38.08 | 6 |
| ATOM | 9612 | C | ILE | C | 799 | 34.631 | 68.369 | 18.464 | 1.00 | 51.09 | 6 |
| ATOM | 9613 | O | ILE | C | 799 | 34.909 | 69.542 | 18.708 | 1.00 | 52.47 | 8 |
| ATOM | 9614 | N | VAL | C | 800 | 34.891 | 67.777 | 17.298 | 1.00 | 24.03 | 7 |
| ATOM | 9615 | CA | VAL | C | 800 | 35.576 | 68.433 | 16.189 | 1.00 | 22.71 | 6 |
| ATOM | 9616 | CB | VAL | C | 800 | 36.247 | 67.384 | 15.296 | 1.00 | 41.32 | 6 |
| ATOM | 9617 | CG1 | VAL | C | 800 | 36.777 | 68.022 | 14.036 | 1.00 | 41.89 | 6 |
| ATOM | 9618 | CG2 | VAL | C | 800 | 37.387 | 66.740 | 16.050 | 1.00 | 43.40 | 6 |
| ATOM | 9619 | C | VAL | C | 800 | 34.703 | 69.317 | 15.317 | 1.00 | 21.31 | 6 |
| ATOM | 9620 | O | VAL | C | 800 | 34.143 | 68.858 | 14.332 | 1.00 | 21.71 | 8 |
| ATOM | 9621 | N | VAL | C | 801 | 34.635 | 70.599 | 15.658 | 1.00 | 31.00 | 7 |
| ATOM | 9622 | CA | VAL | C | 801 | 33.813 | 71.567 | 14.934 | 1.00 | 31.22 | 6 |
| ATOM | 9623 | CB | VAL | C | 801 | 33.840 | 72.913 | 15.645 | 1.00 | 24.39 | 6 |
| ATOM | 9624 | CG1 | VAL | C | 801 | 33.430 | 74.043 | 14.697 | 1.00 | 23.36 | 6 |
| ATOM | 9625 | CG2 | VAL | C | 801 | 32.909 | 72.843 | 16.824 | 1.00 | 22.94 | 6 |
| ATOM | 9626 | C | VAL | C | 801 | 34.071 | 71.802 | 13.455 | 1.00 | 33.09 | 6 |
| ATOM | 9627 | O | VAL | C | 801 | 33.157 | 72.207 | 12.736 | 1.00 | 33.79 | 8 |
| ATOM | 9628 | N | GLY | C | 802 | 35.295 | 71.566 | 12.992 | 1.00 | 57.16 | 7 |
| ATOM | 9629 | CA | GLY | C | 802 | 35.581 | 71.771 | 11.579 | 1.00 | 59.11 | 6 |
| ATOM | 9630 | C | GLY | C | 802 | 37.051 | 71.803 | 11.216 | 1.00 | 60.54 | 6 |
| ATOM | 9631 | O | GLY | C | 802 | 37.899 | 72.121 | 12.050 | 1.00 | 61.00 | 8 |
| ATOM | 9632 | N | ARG | C | 803 | 37.352 | 71.471 | 9.966 | 1.00 | 44.92 | 7 |
| ATOM | 9633 | CA | ARG | C | 803 | 38.727 | 71.469 | 9.486 | 1.00 | 46.07 | 6 |
| ATOM | 9634 | CB | ARG | C | 803 | 39.163 | 70.040 | 9.156 | 1.00 | 68.65 | 6 |
| ATOM | 9635 | CG | ARG | C | 803 | 38.034 | 69.122 | 8.717 | 1.00 | 70.00 | 6 |
| ATOM | 9636 | CD | ARG | C | 803 | 37.239 | 69.702 | 7.543 | 1.00 | 72.16 | 6 |
| ATOM | 9637 | NE | ARG | C | 803 | 36.271 | 68.746 | 6.991 | 1.00 | 74.41 | 7 |
| ATOM | 9638 | CZ | ARG | C | 803 | 35.474 | 68.984 | 5.946 | 1.00 | 74.85 | 6 |
| ATOM | 9639 | NH1 | ARG | C | 803 | 35.510 | 70.155 | 5.320 | 1.00 | 75.42 | 7 |
| ATOM | 9640 | NH2 | ARG | C | 803 | 34.645 | 68.041 | 5.516 | 1.00 | 75.93 | 7 |
| ATOM | 9641 | C | ARG | C | 803 | 38.921 | 72.382 | 8.268 | 1.00 | 46.28 | 6 |
| ATOM | 9642 | O | ARG | C | 803 | 37.956 | 72.907 | 7.710 | 1.00 | 45.62 | 8 |
| ATOM | 9643 | N | LEU | C | 804 | 40.174 | 72.576 | 7.870 | 1.00 | 22.15 | 7 |
| ATOM | 9644 | CA | LEU | C | 804 | 40.498 | 73.426 | 6.734 | 1.00 | 25.08 | 6 |
| ATOM | 9645 | CB | LEU | C | 804 | 40.581 | 74.866 | 7.183 | 1.00 | 42.19 | 6 |
| ATOM | 9646 | CG | LEU | C | 804 | 40.918 | 75.795 | 6.034 | 1.00 | 43.18 | 6 |
| ATOM | 9647 | CD1 | LEU | C | 804 | 39.626 | 76.070 | 5.291 | 1.00 | 42.77 | 6 |
| ATOM | 9648 | CD2 | LEU | C | 804 | 41.565 | 77.089 | 6.544 | 1.00 | 44.40 | 6 |
| ATOM | 9649 | C | LEU | C | 804 | 41.847 | 73.009 | 6.153 | 1.00 | 27.39 | 6 |
| ATOM | 9650 | O | LEU | C | 804 | 42.805 | 72.771 | 6.905 | 1.00 | 27.47 | 8 |
| ATOM | 9651 | N | ARG | C | 805 | 41.922 | 72.955 | 4.820 | 1.00 | 65.05 | 7 |
| ATOM | 9652 | CA | ARG | C | 805 | 43.128 | 72.523 | 4.108 | 1.00 | 67.96 | 6 |
| ATOM | 9653 | CB | ARG | C | 805 | 42.804 | 71.215 | 3.374 | 1.00 | 164.73 | 6 |
| ATOM | 9654 | CG | ARG | C | 805 | 43.967 | 70.269 | 3.112 | 1.00 | 168.40 | 6 |
| ATOM | 9655 | CD | ARG | C | 805 | 43.439 | 68.982 | 2.469 | 1.00 | 171.64 | 6 |
| ATOM | 9656 | NE | ARG | C | 805 | 44.487 | 68.007 | 2.178 | 1.00 | 173.63 | 7 |
| ATOM | 9657 | CZ | ARG | C | 805 | 44.283 | 66.838 | 1.574 | 1.00 | 174.68 | 6 |
| ATOM | 9658 | NH1 | ARG | C | 805 | 43.063 | 66.482 | 1.187 | 1.00 | 175.14 | 7 |
| ATOM | 9659 | NH2 | ARG | C | 805 | 45.302 | 66.018 | 1.354 | 1.00 | 173.89 | 7 |
| ATOM | 9660 | C | ARG | C | 805 | 43.670 | 73.568 | 3.115 | 1.00 | 68.14 | 6 |
| ATOM | 9661 | O | ARG | C | 805 | 42.946 | 74.033 | 2.232 | 1.00 | 68.35 | 8 |
| ATOM | 9662 | N | LEU | C | 806 | 44.950 | 73.919 | 3.262 | 1.00 | 73.86 | 7 |
| ATOM | 9663 | CA | LEU | C | 806 | 45.622 | 74.908 | 2.402 | 1.00 | 75.14 | 6 |
| ATOM | 9664 | CB | LEU | C | 806 | 45.871 | 76.201 | 3.183 | 1.00 | 128.92 | 6 |
| ATOM | 9665 | CG | LEU | C | 806 | 44.675 | 77.043 | 3.619 | 1.00 | 132.18 | 6 |
| ATOM | 9666 | CD1 | LEU | C | 806 | 45.147 | 78.165 | 4.533 | 1.00 | 132.84 | 6 |
| ATOM | 9667 | CD2 | LEU | C | 806 | 43.977 | 77.604 | 2.390 | 1.00 | 133.73 | 6 |
| ATOM | 9668 | C | LEU | C | 806 | 46.972 | 74.417 | 1.858 | 1.00 | 74.67 | 6 |
| ATOM | 9669 | O | LEU | C | 806 | 47.906 | 74.199 | 2.625 | 1.00 | 75.32 | 8 |
| ATOM | 9670 | N | ARG | C | 807 | 47.092 | 74.273 | 0.542 | 1.00 | 48.38 | 7 |
| ATOM | 9671 | CA | ARG | C | 807 | 48.343 | 73.814 | −0.042 | 1.00 | 46.74 | 6 |
| ATOM | 9672 | CB | ARG | C | 807 | 48.237 | 72.331 | −0.397 | 1.00 | 182.04 | 6 |
| ATOM | 9673 | CG | ARG | C | 807 | 47.154 | 72.037 | −1.415 | 1.00 | 186.27 | 6 |
| ATOM | 9674 | CD | ARG | C | 807 | 47.084 | 70.565 | −1.774 | 1.00 | 191.33 | 6 |
| ATOM | 9675 | NE | ARG | C | 807 | 46.168 | 70.349 | −2.890 | 1.00 | 197.20 | 7 |
| ATOM | 9676 | CZ | ARG | C | 807 | 45.906 | 69.164 | −3.434 | 1.00 | 198.77 | 6 |
| ATOM | 9677 | NH1 | ARG | C | 807 | 46.489 | 68.067 | −2.967 | 1.00 | 200.22 | 7 |
| ATOM | 9678 | NH2 | ARG | C | 807 | 45.063 | 69.081 | −4.456 | 1.00 | 199.20 | 7 |
| ATOM | 9679 | C | ARG | C | 807 | 48.715 | 74.609 | −1.290 | 1.00 | 43.90 | 6 |
| ATOM | 9680 | O | ARG | C | 807 | 47.857 | 74.899 | −2.119 | 1.00 | 43.25 | 8 |
| ATOM | 9681 | N | ARG | C | 808 | 49.998 | 74.947 | −1.423 | 1.00 | 21.30 | 7 |
| ATOM | 9682 | CA | ARG | C | 808 | 50.489 | 75.698 | −2.575 | 1.00 | 18.77 | 6 |
| ATOM | 9683 | CB | ARG | C | 808 | 52.018 | 75.756 | −2.596 | 1.00 | 43.57 | 6 |
| ATOM | 9684 | CG | ARG | C | 808 | 52.680 | 74.427 | −2.915 | 1.00 | 42.55 | 6 |
| ATOM | 9685 | CD | ARG | C | 808 | 54.204 | 74.528 | −3.006 | 1.00 | 42.16 | 6 |
| ATOM | 9686 | NE | ARG | C | 808 | 54.838 | 73.290 | −2.541 | 1.00 | 43.97 | 7 |
| ATOM | 9687 | CZ | ARG | C | 808 | 56.149 | 73.063 | −2.530 | 1.00 | 44.91 | 6 |
| ATOM | 9688 | NH1 | ARG | C | 808 | 56.993 | 73.991 | −2.969 | 1.00 | 45.22 | 7 |
| ATOM | 9689 | NH2 | ARG | C | 808 | 56.616 | 71.908 | −2.060 | 1.00 | 45.87 | 7 |
| ATOM | 9690 | C | ARG | C | 808 | 50.011 | 75.034 | −3.847 | 1.00 | 18.28 | 6 |
| ATOM | 9691 | O | ARG | C | 808 | 49.821 | 73.821 | −3.895 | 1.00 | 16.07 | 8 |
| ATOM | 9692 | N | GLY | C | 809 | 49.856 | 75.838 | −4.889 | 1.00 | 68.74 | 7 |
| ATOM | 9693 | CA | GLY | C | 809 | 49.364 | 75.329 | −6.149 | 1.00 | 70.46 | 6 |
| ATOM | 9694 | C | GLY | C | 809 | 47.946 | 75.832 | −6.122 | 1.00 | 71.55 | 6 |
| ATOM | 9695 | O | GLY | C | 809 | 47.315 | 76.039 | −7.154 | 1.00 | 73.19 | 8 |
| ATOM | 9696 | N | ASP | C | 810 | 47.461 | 76.032 | −4.900 | 1.00 | 57.25 | 7 |
| ATOM | 9697 | CA | ASP | C | 810 | 46.123 | 76.549 | −4.644 | 1.00 | 57.46 | 6 |
| ATOM | 9698 | CB | ASP | C | 810 | 45.809 | 76.450 | −3.145 | 1.00 | 46.40 | 6 |
| ATOM | 9699 | CG | ASP | C | 810 | 44.543 | 75.679 | −2.850 | 1.00 | 46.28 | 6 |
| ATOM | 9700 | OD1 | ASP | C | 810 | 43.542 | 75.868 | −3.567 | 1.00 | 47.68 | 8 |
| ATOM | 9701 | OD2 | ASP | C | 810 | 44.538 | 74.894 | −1.881 | 1.00 | 45.83 | 8 |
| ATOM | 9702 | C | ASP | C | 810 | 46.220 | 78.022 | −5.040 | 1.00 | 58.13 | 6 |
| ATOM | 9703 | O | ASP | C | 810 | 47.312 | 78.501 | −5.352 | 1.00 | 58.06 | 8 |
| ATOM | 9704 | N | PRO | C | 811 | 45.093 | 78.757 | −5.028 | 1.00 | 83.43 | 7 |
| ATOM | 9705 | CD | PRO | C | 811 | 43.732 | 78.324 | −4.698 | 1.00 | 44.68 | 6 |
| ATOM | 9706 | CA | PRO | C | 811 | 45.070 | 80.177 | −5.382 | 1.00 | 85.22 | 6 |
| ATOM | 9707 | CB | PRO | C | 811 | 43.594 | 80.511 | −5.337 | 1.00 | 45.44 | 6 |
| ATOM | 9708 | CG | PRO | C | 811 | 43.115 | 79.628 | −4.272 | 1.00 | 44.85 | 6 |
| ATOM | 9709 | C | PRO | C | 811 | 45.862 | 81.063 | −4.429 | 1.00 | 87.07 | 6 |
| ATOM | 9710 | O | PRO | C | 811 | 46.932 | 80.684 | −3.956 | 1.00 | 88.35 | 8 |
| ATOM | 9711 | N | GLY | C | 812 | 45.311 | 82.243 | −4.153 | 1.00 | 91.85 | 7 |
| ATOM | 9712 | CA | GLY | C | 812 | 45.960 | 83.213 | −3.286 | 1.00 | 92.81 | 6 |
| ATOM | 9713 | C | GLY | C | 812 | 47.067 | 82.698 | −2.386 | 1.00 | 91.54 | 6 |
| ATOM | 9714 | O | GLY | C | 812 | 48.159 | 83.259 | −2.334 | 1.00 | 92.22 | 8 |
| ATOM | 9715 | N | VAL | C | 813 | 46.775 | 81.612 | −1.686 | 1.00 | 43.41 | 7 |
| ATOM | 9716 | CA | VAL | C | 813 | 47.690 | 80.985 | −0.745 | 1.00 | 41.37 | 6 |
| ATOM | 9717 | CB | VAL | C | 813 | 47.395 | 79.458 | −0.647 | 1.00 | 26.32 | 6 |
| ATOM | 9718 | CG1 | VAL | C | 813 | 47.752 | 78.952 | 0.745 | 1.00 | 25.82 | 6 |
| ATOM | 9719 | CG2 | VAL | C | 813 | 45.907 | 79.178 | −0.947 | 1.00 | 25.69 | 6 |
| ATOM | 9720 | C | VAL | C | 813 | 49.189 | 81.201 | −0.995 | 1.00 | 41.50 | 6 |
| ATOM | 9721 | O | VAL | C | 813 | 49.753 | 80.756 | −1.992 | 1.00 | 40.43 | 8 |
| ATOM | 9722 | N | GLU | C | 814 | 49.824 | 81.919 | −0.080 | 1.00 | 34.98 | 7 |
| ATOM | 9723 | CA | GLU | C | 814 | 51.252 | 82.166 | −0.149 | 1.00 | 37.19 | 6 |
| ATOM | 9724 | CB | GLU | C | 814 | 51.558 | 83.654 | 0.097 | 1.00 | 115.90 | 6 |
| ATOM | 9725 | CG | CLU | C | 814 | 51.185 | 84.161 | 1.496 | 1.00 | 119.46 | 6 |
| ATOM | 9726 | CD | GLU | C | 814 | 51.402 | 85.658 | 1.686 | 1.00 | 121.07 | 6 |
| ATOM | 9727 | OE1 | GLU | C | 814 | 50.693 | 86.450 | 1.033 | 1.00 | 122.38 | 8 |
| ATOM | 9728 | OE2 | GLU | C | 814 | 52.277 | 86.040 | 2.494 | 1.00 | 122.43 | 8 |
| ATOM | 9729 | C | GLU | C | 814 | 51.717 | 81.319 | 1.026 | 1.00 | 37.29 | 6 |
| ATOM | 9730 | O | GLU | C | 814 | 51.220 | 81.483 | 2.140 | 1.00 | 37.09 | 8 |
| ATOM | 9731 | N | LEU | C | 815 | 52.637 | 80.390 | 0.806 | 1.00 | 28.62 | 7 |
| ATOM | 9732 | CA | LEU | C | 815 | 53.052 | 79.565 | 1.931 | 1.00 | 29.31 | 6 |
| ATOM | 9733 | CB | LEU | C | 815 | 52.386 | 78.192 | 1.855 | 1.00 | 36.74 | 6 |
| ATOM | 9734 | CG | LEU | C | 815 | 50.882 | 78.194 | 1.590 | 1.00 | 35.82 | 6 |
| ATOM | 9735 | CD1 | LEU | C | 815 | 50.660 | 78.503 | 0.126 | 1.00 | 36.39 | 6 |
| ATOM | 9736 | CD2 | LEU | C | 815 | 50.276 | 76.848 | 1.918 | 1.00 | 35.80 | 6 |
| ATOM | 9737 | C | LEU | C | 815 | 54.548 | 79.392 | 2.070 | 1.00 | 30.73 | 6 |
| ATOM | 9738 | O | LEU | C | 815 | 55.253 | 79.147 | 1.092 | 1.00 | 29.93 | 8 |
| ATOM | 9739 | N | LYS | C | 816 | 55.000 | 79.526 | 3.317 | 1.00 | 99.29 | 7 |
| ATOM | 9740 | CA | LYS | C | 816 | 56.399 | 79.400 | 3.723 | 1.00 | 101.20 | 6 |
| ATOM | 9741 | CB | LYS | C | 816 | 56.472 | 78.895 | 5.170 | 1.00 | 129.01 | 6 |
| ATOM | 9742 | CG | LYS | C | 816 | 57.793 | 78.249 | 5.580 | 1.00 | 130.38 | 6 |
| ATOM | 9743 | CD | LYS | C | 816 | 58.960 | 79.214 | 5.587 | 1.00 | 131.78 | 6 |
| ATOM | 9744 | CE | LYS | C | 816 | 60.218 | 78.526 | 6.102 | 1.00 | 132.53 | 6 |
| ATOM | 9745 | NZ | LYS | C | 816 | 60.544 | 77.289 | 5.335 | 1.00 | 132.71 | 7 |
| ATOM | 9746 | C | LYS | C | 816 | 57.173 | 78.474 | 2.814 | 1.00 | 101.91 | 6 |
| ATOM | 9747 | O | LYS | C | 816 | 56.805 | 77.311 | 2.639 | 1.00 | 102.19 | 8 |
| ATOM | 9748 | N | PRO | C | 817 | 58.267 | 78.983 | 2.226 | 1.00 | 85.14 | 7 |
| ATOM | 9749 | CD | PRO | C | 817 | 58.920 | 80.268 | 2.522 | 1.00 | 123.43 | 6 |
| ATOM | 9750 | CA | PRO | C | 817 | 59.094 | 78.180 | 1.322 | 1.00 | 85.29 | 6 |
| ATOM | 9751 | CB | PRO | C | 817 | 60.424 | 78.942 | 1.291 | 1.00 | 124.09 | 6 |
| ATOM | 9752 | CG | PRO | C | 817 | 60.355 | 79.875 | 2.498 | 1.00 | 124.53 | 6 |
| ATOM | 9753 | C | PRO | C | 817 | 59.232 | 76.731 | 1.774 | 1.00 | 85.00 | 6 |
| ATOM | 9754 | O | PRO | C | 817 | 59.646 | 76.447 | 2.905 | 1.00 | 84.81 | 8 |
| ATOM | 9755 | N | GLY | C | 818 | 58.858 | 75.821 | 0.881 | 1.00 | 111.88 | 7 |
| ATOM | 9756 | CA | GLY | C | 818 | 58.929 | 74.409 | 1.189 | 1.00 | 111.02 | 6 |
| ATOM | 9757 | C | GLY | C | 818 | 57.614 | 73.916 | 1.752 | 1.00 | 109.73 | 6 |
| ATOM | 9758 | O | GLY | C | 818 | 57.097 | 72.879 | 1.336 | 1.00 | 110.63 | 8 |
| ATOM | 9759 | N | VAL | C | 819 | 57.068 | 74.661 | 2.704 | 1.00 | 88.15 | 7 |
| ATOM | 9760 | CA | VAL | C | 819 | 55.807 | 74.284 | 3.311 | 1.00 | 85.63 | 6 |
| ATOM | 9761 | CB | VAL | C | 819 | 55.234 | 75.421 | 4.159 | 1.00 | 96.98 | 6 |
| ATOM | 9762 | CG1 | VAL | C | 819 | 53.832 | 75.072 | 4.619 | 1.00 | 98.99 | 6 |
| ATOM | 9763 | CG2 | VAL | C | 819 | 56.118 | 75.665 | 5.344 | 1.00 | 96.50 | 6 |
| ATOM | 9764 | C | VAL | C | 819 | 54.798 | 73.947 | 2.232 | 1.00 | 83.05 | 6 |
| ATOM | 9765 | O | VAL | C | 819 | 54.316 | 74.836 | 1.536 | 1.00 | 82.90 | 8 |
| ATOM | 9766 | N | ARG | C | 820 | 54.498 | 72.662 | 2.080 | 1.00 | 43.94 | 7 |
| ATOM | 9767 | CA | ARG | C | 820 | 53.518 | 72.215 | 1.097 | 1.00 | 41.90 | 6 |
| ATOM | 9768 | CB | ARG | C | 820 | 54.029 | 70.970 | 0.360 | 1.00 | 120.94 | 6 |
| ATOM | 9769 | CG | ARG | C | 820 | 53.111 | 70.480 | −0.758 | 1.00 | 126.07 | 6 |
| ATOM | 9770 | CD | ARG | C | 820 | 53.519 | 69.097 | −1.285 | 1.00 | 129.75 | 6 |
| ATOM | 9771 | NE | ARG | C | 820 | 52.627 | 68.640 | −2.354 | 1.00 | 133.10 | 7 |
| ATOM | 9772 | CZ | ARG | C | 820 | 52.639 | 67.417 | −2.881 | 1.00 | 134.05 | 6 |
| ATOM | 9773 | NH1 | ARG | C | 820 | 53.497 | 66.506 | −2.442 | 1.00 | 134.60 | 7 |
| ATOM | 9774 | NH2 | ARG | C | 820 | 51.793 | 67.102 | −3.851 | 1.00 | 133.74 | 7 |
| ATOM | 9775 | C | ARG | C | 820 | 52.333 | 71.859 | 1.966 | 1.00 | 38.35 | 6 |
| ATOM | 9776 | O | ARG | C | 820 | 52.510 | 71.210 | 2.987 | 1.00 | 37.36 | 8 |
| ATOM | 9777 | N | GLU | C | 821 | 51.137 | 72.293 | 1.597 | 1.00 | 46.48 | 7 |
| ATOM | 9778 | CA | GLU | C | 821 | 49.968 | 71.984 | 2.408 | 1.00 | 43.14 | 6 |
| ATOM | 9779 | CB | GLU | C | 821 | 49.680 | 70.491 | 2.335 | 1.00 | 22.16 | 6 |
| ATOM | 9780 | CG | GLU | C | 821 | 48.402 | 70.105 | 3.011 | 1.00 | 23.51 | 6 |
| ATOM | 9781 | CD | GLU | C | 821 | 47.944 | 68.704 | 2.652 | 1.00 | 25.58 | 6 |
| ATOM | 9782 | OE1 | GLU | C | 821 | 48.733 | 67.751 | 2.836 | 1.00 | 27.22 | 8 |
| ATOM | 9783 | OE2 | GLU | C | 821 | 46.790 | 68.550 | 2.190 | 1.00 | 25.44 | 8 |
| ATOM | 9784 | C | GLU | C | 821 | 50.133 | 72.417 | 3.879 | 1.00 | 41.31 | 6 |
| ATOM | 9785 | O | GLU | C | 821 | 51.230 | 72.381 | 4.422 | 1.00 | 41.76 | 8 |
| ATOM | 9786 | N | VAL | C | 822 | 49.030 | 72.825 | 4.511 | 1.00 | 32.88 | 7 |
| ATOM | 9787 | CA | VAL | C | 822 | 49.010 | 73.290 | 5.909 | 1.00 | 28.33 | 6 |
| ATOM | 9788 | CB | VAL | C | 822 | 49.221 | 74.816 | 5.983 | 1.00 | 17.02 | 6 |
| ATOM | 9789 | CG1 | VAL | C | 822 | 49.036 | 75.311 | 7.409 | 1.00 | 15.41 | 6 |
| ATOM | 9790 | CG2 | VAL | C | 822 | 50.609 | 75.162 | 5.471 | 1.00 | 15.95 | 6 |
| ATOM | 9791 | C | VAL | C | 822 | 47.664 | 72.979 | 6.559 | 1.00 | 26.88 | 6 |
| ATOM | 9792 | O | VAL | C | 822 | 46.785 | 73.817 | 6.583 | 1.00 | 28.37 | 8 |
| ATOM | 9793 | N | VAL | C | 823 | 47.507 | 71.780 | 7.095 | 1.00 | 24.67 | 7 |
| ATOM | 9794 | CA | VAL | C | 823 | 46.249 | 71.378 | 7.708 | 1.00 | 24.23 | 6 |
| ATOM | 9795 | CB | VAL | C | 823 | 46.308 | 69.878 | 8.123 | 1.00 | 15.35 | 6 |
| ATOM | 9796 | CG1 | VAL | C | 823 | 45.004 | 69.435 | 8.776 | 1.00 | 15.07 | 6 |
| ATOM | 9797 | CG2 | VAL | C | 823 | 46.581 | 69.035 | 6.912 | 1.00 | 16.46 | 6 |
| ATOM | 9798 | C | VAL | C | 823 | 45.880 | 72.211 | 8.937 | 1.00 | 25.40 | 6 |
| ATOM | 9799 | O | VAL | C | 823 | 46.754 | 72.594 | 9.729 | 1.00 | 26.38 | 8 |
| ATOM | 9800 | N | ARG | C | 824 | 44.581 | 72.488 | 9.081 | 1.00 | 37.23 | 7 |
| ATOM | 9801 | CA | ARG | C | 824 | 44.048 | 73.217 | 10.232 | 1.00 | 36.42 | 6 |
| ATOM | 9802 | CB | ARG | C | 824 | 43.706 | 74.656 | 9.876 | 1.00 | 41.29 | 6 |
| ATOM | 9803 | CG | ARG | C | 824 | 44.900 | 75.561 | 9.769 | 1.00 | 41.99 | 6 |
| ATOM | 9804 | CD | ARG | C | 824 | 44.451 | 76.955 | 9.424 | 1.00 | 44.29 | 6 |
| ATOM | 9805 | NE | ARG | C | 824 | 45.567 | 77.790 | 9.001 | 1.00 | 46.91 | 7 |
| ATOM | 9806 | CZ | ARG | C | 824 | 45.444 | 79.061 | 8.635 | 1.00 | 47.62 | 6 |
| ATOM | 9807 | NH1 | ARG | C | 824 | 44.245 | 79.643 | 8.642 | 1.00 | 48.05 | 7 |
| ATOM | 9808 | NH2 | ARG | C | 824 | 46.520 | 79.747 | 8.269 | 1.00 | 48.13 | 7 |
| ATOM | 9809 | C | ARG | C | 824 | 42.784 | 72.525 | 10.704 | 1.00 | 35.88 | 6 |
| ATOM | 9810 | O | ARG | C | 824 | 41.932 | 72.168 | 9.888 | 1.00 | 35.49 | 8 |
| ATOM | 9811 | N | VAL | C | 825 | 42.664 | 72.337 | 12.016 | 1.00 | 38.85 | 7 |
| ATOM | 9812 | CA | VAL | C | 825 | 41.490 | 71.691 | 12.593 | 1.00 | 36.68 | 6 |
| ATOM | 9813 | CB | VAL | C | 825 | 41.779 | 70.208 | 12.898 | 1.00 | 13.87 | 6 |
| ATOM | 9814 | CG1 | VAL | C | 825 | 40.641 | 69.584 | 13.710 | 1.00 | 13.87 | 6 |
| ATOM | 9815 | CG2 | VAL | C | 825 | 41.939 | 69.463 | 11.610 | 1.00 | 13.87 | 6 |
| ATOM | 9816 | C | VAL | C | 825 | 41.003 | 72.386 | 13.868 | 1.00 | 37.58 | 6 |
| ATOM | 9817 | O | VAL | C | 825 | 41.771 | 72.516 | 14.827 | 1.00 | 37.01 | 8 |
| ATOM | 9818 | N | PHE | C | 826 | 39.734 | 72.828 | 13.856 | 1.00 | 25.93 | 7 |
| ATOM | 9819 | CA | PHE | C | 826 | 39.096 | 73.502 | 14.994 | 1.00 | 24.67 | 6 |
| ATOM | 9820 | CB | PHE | C | 826 | 38.096 | 74.543 | 14.535 | 1.00 | 16.41 | 6 |
| ATOM | 9821 | CG | PHE | C | 826 | 38.639 | 75.545 | 13.590 | 1.00 | 16.49 | 6 |
| ATOM | 9822 | CD1 | PHE | C | 826 | 39.872 | 76.125 | 13.802 | 1.00 | 17.42 | 6 |
| ATOM | 9823 | CD2 | PHE | C | 826 | 37.877 | 75.970 | 12.515 | 1.00 | 16.81 | 6 |
| ATOM | 9824 | CE1 | PHE | C | 826 | 40.345 | 77.122 | 12.958 | 1.00 | 16.74 | 6 |
| ATOM | 9825 | CE2 | PHE | C | 826 | 38.337 | 76.962 | 11.669 | 1.00 | 16.62 | 6 |
| ATOM | 9826 | CZ | PHE | C | 826 | 39.582 | 77.541 | 11.897 | 1.00 | 17.57 | 6 |
| ATOM | 9827 | C | PHE | C | 826 | 38.316 | 72.520 | 15.854 | 1.00 | 25.21 | 6 |
| ATOM | 9828 | O | PHE | C | 826 | 37.588 | 71.685 | 15.325 | 1.00 | 24.96 | 8 |
| ATOM | 9829 | N | VAL | C | 827 | 38.439 | 72.655 | 17.174 | 1.00 | 27.15 | 7 |
| ATOM | 9830 | CA | VAL | C | 827 | 37.754 | 71.785 | 18.134 | 1.00 | 28.02 | 6 |
| ATOM | 9831 | CB | VAL | C | 827 | 38.748 | 71.002 | 18.998 | 1.00 | 20.05 | 6 |
| ATOM | 9832 | CG1 | VAL | C | 827 | 38.042 | 70.373 | 20.152 | 1.00 | 21.05 | 6 |
| ATOM | 9833 | CG2 | VAL | C | 827 | 39.393 | 69.929 | 18.192 | 1.00 | 22.40 | 6 |
| ATOM | 9834 | C | VAL | C | 827 | 36.936 | 72.635 | 19.073 | 1.00 | 29.38 | 6 |
| ATOM | 9835 | O | VAL | C | 827 | 37.424 | 73.643 | 19.548 | 1.00 | 32.32 | 8 |
| ATOM | 9836 | N | ALA | C | 828 | 35.706 | 72.231 | 19.358 | 1.00 | 23.27 | 7 |
| ATOM | 9837 | CA | ALA | C | 828 | 34.872 | 73.011 | 20.253 | 1.00 | 24.00 | 6 |
| ATOM | 9838 | CB | ALA | C | 828 | 33.423 | 72.947 | 19.810 | 1.00 | 13.87 | 6 |
| ATOM | 9839 | C | ALA | C | 828 | 35.015 | 72.477 | 21.660 | 1.00 | 25.87 | 6 |
| ATOM | 9840 | O | ALA | C | 828 | 35.602 | 71.422 | 21.855 | 1.00 | 26.88 | 8 |
| ATOM | 9841 | N | GLN | C | 829 | 34.468 | 73.206 | 22.634 | 1.00 | 44.45 | 7 |
| ATOM | 9842 | CA | GLN | C | 829 | 34.525 | 72.806 | 24.042 | 1.00 | 46.16 | 6 |
| ATOM | 9843 | CB | GLN | C | 829 | 35.925 | 73.080 | 24.591 | 1.00 | 25.13 | 6 |
| ATOM | 9844 | CG | GLN | C | 829 | 36.639 | 71.870 | 25.163 | 1.00 | 26.51 | 6 |
| ATOM | 9845 | CD | GLN | C | 829 | 38.022 | 72.218 | 25.698 | 1.00 | 27.53 | 6 |
| ATOM | 9846 | OE1 | GLN | C | 829 | 38.787 | 72.934 | 25.048 | 1.00 | 27.91 | 8 |
| ATOM | 9847 | NE2 | GLN | C | 829 | 38.353 | 71.700 | 26.880 | 1.00 | 28.79 | 7 |
| ATOM | 9848 | C | GLN | C | 829 | 33.489 | 73.524 | 24.934 | 1.00 | 47.54 | 6 |
| ATOM | 9849 | O | GLN | C | 829 | 33.752 | 74.611 | 25.437 | 1.00 | 48.43 | 8 |
| ATOM | 9850 | N | LYS | C | 830 | 32.313 | 72.930 | 25.124 | 1.00 | 55.30 | 7 |
| ATOM | 9851 | CA | LYS | C | 830 | 31.291 | 73.524 | 25.992 | 1.00 | 55.79 | 6 |
| ATOM | 9852 | CB | LYS | C | 830 | 30.049 | 72.645 | 26.046 | 1.00 | 44.08 | 6 |
| ATOM | 9853 | CG | LYS | C | 830 | 28.730 | 73.330 | 25.813 | 1.00 | 44.08 | 6 |
| ATOM | 9854 | CD | LYS | C | 830 | 27.638 | 72.319 | 26.135 | 1.00 | 44.95 | 6 |
| ATOM | 9855 | CE | LYS | C | 830 | 26.243 | 72.782 | 25.733 | 1.00 | 44.96 | 6 |
| ATOM | 9856 | NZ | LYS | C | 830 | 25.185 | 71.813 | 26.191 | 1.00 | 46.02 | 7 |
| ATOM | 9857 | C | LYS | C | 830 | 31.990 | 73.449 | 27.333 | 1.00 | 56.00 | 6 |
| ATOM | 9858 | O | LYS | C | 830 | 32.352 | 72.356 | 27.776 | 1.00 | 57.65 | 8 |
| ATOM | 9859 | N | ARG | C | 831 | 32.164 | 74.586 | 27.995 | 1.00 | 37.74 | 7 |
| ATOM | 9860 | CA | ARG | C | 831 | 32.890 | 74.584 | 29.247 | 1.00 | 38.23 | 6 |
| ATOM | 9861 | CB | ARG | C | 831 | 34.073 | 75.539 | 29.095 | 1.00 | 32.53 | 6 |
| ATOM | 9862 | CG | ARG | C | 831 | 35.225 | 74.934 | 28.313 | 1.00 | 32.88 | 6 |
| ATOM | 9863 | CD | ARG | C | 831 | 36.136 | 75.977 | 27.686 | 1.00 | 32.47 | 6 |
| ATOM | 9864 | NE | ARG | C | 831 | 37.389 | 75.382 | 27.231 | 1.00 | 31.19 | 7 |
| ATOM | 9865 | CZ | ARG | C | 831 | 38.425 | 75.146 | 28.028 | 1.00 | 30.93 | 6 |
| ATOM | 9866 | NH1 | ARG | C | 831 | 38.354 | 75.466 | 29.310 | 1.00 | 28.35 | 7 |
| ATOM | 9867 | NH2 | ARG | C | 831 | 39.522 | 74.579 | 27.549 | 1.00 | 32.06 | 7 |
| ATOM | 9868 | C | ARG | C | 831 | 32.207 | 74.804 | 30.600 | 1.00 | 38.44 | 6 |
| ATOM | 9869 | O | ARG | C | 831 | 32.888 | 75.107 | 31.564 | 1.00 | 40.82 | 8 |
| ATOM | 9870 | N | LYS | C | 832 | 30.897 | 74.621 | 30.697 | 1.00 | 54.72 | 7 |
| ATOM | 9871 | CA | LYS | C | 832 | 30.190 | 74.803 | 31.971 | 1.00 | 53.39 | 6 |
| ATOM | 9872 | CB | LYS | C | 832 | 29.877 | 73.451 | 32.614 | 1.00 | 53.99 | 6 |
| ATOM | 9873 | CG | LYS | C | 832 | 29.311 | 72.425 | 31.643 | 1.00 | 58.08 | 6 |
| ATOM | 9874 | CD | LYS | C | 832 | 28.366 | 71.424 | 32.304 | 1.00 | 59.20 | 6 |
| ATOM | 9875 | CE | LYS | C | 832 | 27.080 | 72.113 | 32.715 | 1.00 | 59.55 | 6 |
| ATOM | 9876 | NZ | LYS | C | 832 | 26.026 | 71.157 | 33.141 | 1.00 | 61.57 | 7 |
| ATOM | 9877 | C | LYS | C | 832 | 30.942 | 75.668 | 32.987 | 1.00 | 52.37 | 6 |
| ATOM | 9878 | O | LYS | C | 832 | 31.604 | 76.647 | 32.620 | 1.00 | 53.67 | 8 |
| ATOM | 9879 | N | LEU | C | 833 | 30.842 | 75.314 | 34.266 | 1.00 | 22.03 | 7 |
| ATOM | 9880 | CA | LEU | C | 833 | 31.512 | 76.088 | 35.306 | 1.00 | 19.43 | 6 |
| ATOM | 9881 | CB | LEU | C | 833 | 30.513 | 77.081 | 35.920 | 1.00 | 13.87 | 6 |
| ATOM | 9882 | CG | LEU | C | 833 | 31.006 | 78.195 | 36.844 | 1.00 | 13.87 | 6 |
| ATOM | 9883 | CD1 | LEU | C | 833 | 32.109 | 78.938 | 36.145 | 1.00 | 13.87 | 6 |
| ATOM | 9884 | CD2 | LEU | C | 833 | 29.872 | 79.142 | 37.230 | 1.00 | 13.87 | 6 |
| ATOM | 9885 | C | LEU | C | 833 | 32.138 | 75.182 | 36.381 | 1.00 | 18.99 | 6 |
| ATOM | 9886 | O | LEU | C | 833 | 31.473 | 74.316 | 36.965 | 1.00 | 18.69 | 8 |
| ATOM | 9887 | N | GLN | C | 834 | 33.430 | 75.389 | 36.638 | 1.00 | 28.50 | 7 |
| ATOM | 9888 | CA | GLN | C | 834 | 34.149 | 74.585 | 37.620 | 1.00 | 28.53 | 6 |
| ATOM | 9889 | CB | GLN | C | 834 | 35.226 | 73.770 | 36.917 | 1.00 | 68.17 | 6 |
| ATOM | 9890 | CG | GLN | C | 834 | 34.823 | 73.247 | 35.564 | 1.00 | 72.95 | 6 |
| ATOM | 9891 | CD | GLN | C | 834 | 35.890 | 72.354 | 34.980 | 1.00 | 75.96 | 6 |
| ATOM | 9892 | OE1 | GLN | C | 834 | 37.039 | 72.767 | 34.815 | 1.00 | 76.56 | 8 |
| ATOM | 9893 | NE2 | GLN | C | 834 | 35.520 | 71.115 | 34.666 | 1.00 | 77.61 | 7 |
| ATOM | 9894 | C | GLN | C | 834 | 34.805 | 75.371 | 38.754 | 1.00 | 25.06 | 6 |
| ATOM | 9895 | O | GLN | C | 834 | 34.796 | 76.597 | 38.781 | 1.00 | 24.26 | 8 |
| ATOM | 9896 | N | VAL | C | 835 | 35.392 | 74.623 | 39.680 | 1.00 | 13.87 | 7 |
| ATOM | 9897 | CA | VAL | C | 835 | 36.079 | 75.172 | 40.827 | 1.00 | 13.87 | 6 |
| ATOM | 9898 | CB | VAL | C | 835 | 36.346 | 74.104 | 41.828 | 1.00 | 14.75 | 6 |
| ATOM | 9899 | CG1 | VAL | C | 835 | 37.114 | 74.666 | 42.981 | 1.00 | 14.75 | 6 |
| ATOM | 9900 | CG2 | VAL | C | 835 | 35.053 | 73.524 | 42.271 | 1.00 | 16.67 | 6 |
| ATOM | 9901 | C | VAL | C | 835 | 37.407 | 75.791 | 40.449 | 1.00 | 13.87 | 6 |
| ATOM | 9902 | O | VAL | C | 835 | 38.408 | 75.104 | 40.255 | 1.00 | 13.87 | 8 |
| ATOM | 9903 | N | GLY | C | 836 | 37.398 | 77.112 | 40.382 | 1.00 | 38.13 | 7 |
| ATOM | 9904 | CA | GLY | C | 836 | 38.561 | 77.895 | 40.014 | 1.00 | 38.87 | 6 |
| ATOM | 9905 | C | GLY | C | 836 | 37.859 | 79.068 | 39.388 | 1.00 | 39.59 | 6 |
| ATOM | 9906 | O | GLY | C | 836 | 36.947 | 79.625 | 39.999 | 1.00 | 40.37 | 8 |
| ATOM | 9907 | N | ASP | C | 837 | 38.246 | 79.440 | 38.180 | 1.00 | 15.05 | 7 |
| ATOM | 9908 | CA | ASP | C | 837 | 37.571 | 80.543 | 37.470 | 1.00 | 17.20 | 6 |
| ATOM | 9909 | CB | ASP | C | 837 | 36.283 | 80.050 | 36.758 | 1.00 | 30.43 | 6 |
| ATOM | 9910 | CG | ASP | C | 837 | 36.204 | 78.534 | 36.625 | 1.00 | 30.90 | 6 |
| ATOM | 9911 | OD1 | ASP | C | 837 | 35.425 | 78.057 | 35.779 | 1.00 | 31.21 | 8 |
| ATOM | 9912 | OD2 | ASP | C | 837 | 36.896 | 77.810 | 37.365 | 1.00 | 31.70 | 8 |
| ATOM | 9913 | C | ASP | C | 837 | 37.195 | 81.840 | 38.229 | 1.00 | 17.18 | 6 |
| ATOM | 9914 | O | ASP | C | 837 | 36.721 | 81.837 | 39.379 | 1.00 | 15.72 | 8 |
| ATOM | 9915 | N | LYS | C | 838 | 37.393 | 82.956 | 37.537 | 1.00 | 51.34 | 7 |
| ATOM | 9916 | CA | LYS | C | 838 | 37.074 | 84.250 | 38.094 | 1.00 | 50.43 | 6 |
| ATOM | 9917 | CB | LYS | C | 838 | 38.029 | 85.307 | 37.536 | 1.00 | 41.17 | 6 |
| ATOM | 9918 | CG | LYS | C | 838 | 39.457 | 85.176 | 38.068 | 1.00 | 43.09 | 6 |
| ATOM | 9919 | CD | LYS | C | 838 | 40.381 | 86.221 | 37.470 | 1.00 | 44.30 | 6 |
| ATOM | 9920 | CE | LYS | C | 838 | 39.827 | 87.606 | 37.648 | 1.00 | 45.38 | 6 |
| ATOM | 9921 | NZ | LYS | C | 838 | 40.582 | 88.498 | 36.754 | 1.00 | 47.80 | 7 |
| ATOM | 9922 | C | LYS | C | 838 | 35.630 | 84.584 | 37.755 | 1.00 | 49.10 | 6 |
| ATOM | 9923 | O | LYS | C | 838 | 35.193 | 84.431 | 36.615 | 1.00 | 50.63 | 8 |
| ATOM | 9924 | N | LEU | C | 839 | 34.880 | 85.022 | 38.754 | 1.00 | 21.50 | 7 |
| ATOM | 9925 | CA | LEU | C | 839 | 33.500 | 85.352 | 38.523 | 1.00 | 18.88 | 6 |
| ATOM | 9926 | CB | LEU | C | 839 | 32.654 | 84.948 | 39.721 | 1.00 | 13.87 | 6 |
| ATOM | 9927 | CG | LEU | C | 839 | 31.429 | 84.085 | 39.391 | 1.00 | 13.87 | 6 |
| ATOM | 9928 | CD1 | LEU | C | 839 | 30.827 | 84.533 | 38.074 | 1.00 | 13.87 | 6 |
| ATOM | 9929 | CD2 | LEU | C | 839 | 31.826 | 82.633 | 39.302 | 1.00 | 13.87 | 6 |
| ATOM | 9930 | C | LEU | C | 839 | 33.403 | 86.837 | 38.258 | 1.00 | 18.99 | 6 |
| ATOM | 9931 | O | LEU | C | 839 | 32.684 | 87.258 | 37.372 | 1.00 | 19.95 | 8 |
| ATOM | 9932 | N | ALA | C | 840 | 34.109 | 87.634 | 39.038 | 1.00 | 13.87 | 7 |
| ATOM | 9933 | CA | ALA | C | 840 | 34.136 | 89.074 | 38.821 | 1.00 | 13.87 | 6 |
| ATOM | 9934 | CB | ALA | C | 840 | 35.382 | 89.406 | 38.064 | 1.00 | 95.91 | 6 |
| ATOM | 9935 | C | ALA | C | 840 | 32.946 | 89.759 | 38.133 | 1.00 | 13.87 | 6 |
| ATOM | 9936 | O | ALA | C | 840 | 32.643 | 89.504 | 36.970 | 1.00 | 13.87 | 8 |
| ATOM | 9937 | N | ASN | C | 841 | 32.308 | 90.676 | 38.858 | 1.00 | 49.24 | 7 |
| ATOM | 9938 | CA | ASN | C | 841 | 31.163 | 91.419 | 38.341 | 1.00 | 50.01 | 6 |
| ATOM | 9939 | CB | ASN | C | 841 | 30.166 | 91.723 | 39.451 | 1.00 | 20.39 | 6 |
| ATOM | 9940 | CG | ASN | C | 841 | 30.715 | 92.659 | 40.486 | 1.00 | 18.55 | 6 |
| ATOM | 9941 | OD1 | ASN | C | 841 | 30.110 | 92.836 | 41.543 | 1.00 | 18.43 | 8 |
| ATOM | 9942 | ND2 | ASN | C | 841 | 31.860 | 93.269 | 40.199 | 1.00 | 18.43 | 7 |
| ATOM | 9943 | C | ASN | C | 841 | 31.571 | 92.721 | 37.683 | 1.00 | 49.72 | 6 |
| ATOM | 9944 | O | ASN | C | 841 | 32.631 | 92.800 | 37.068 | 1.00 | 52.22 | 8 |
| ATOM | 9945 | N | ARG | C | 842 | 30.731 | 93.745 | 37.841 | 1.00 | 27.48 | 7 |
| ATOM | 9946 | CA | ARG | C | 842 | 30.977 | 95.047 | 37.221 | 1.00 | 27.33 | 6 |
| ATOM | 9947 | CB | ARG | C | 842 | 29.660 | 95.649 | 36.717 | 1.00 | 23.83 | 6 |
| ATOM | 9948 | CG | ARG | C | 842 | 29.075 | 94.961 | 35.490 | 1.00 | 23.93 | 6 |
| ATOM | 9949 | CD | ARG | C | 842 | 27.774 | 95.599 | 35.065 | 1.00 | 23.24 | 6 |
| ATOM | 9950 | NE | ARG | C | 842 | 26.792 | 95.541 | 36.140 | 1.00 | 22.83 | 7 |
| ATOM | 9951 | CZ | ARG | C | 842 | 25.509 | 95.839 | 35.985 | 1.00 | 23.87 | 6 |
| ATOM | 9952 | NH1 | ARG | C | 842 | 25.070 | 96.213 | 34.795 | 1.00 | 23.14 | 7 |
| ATOM | 9953 | NH2 | ARG | C | 842 | 24.672 | 95.759 | 37.010 | 1.00 | 23.58 | 7 |
| ATOM | 9954 | C | ARG | C | 842 | 31.680 | 96.063 | 38.095 | 1.00 | 27.01 | 6 |
| ATOM | 9955 | O | ARG | C | 842 | 32.464 | 96.857 | 37.603 | 1.00 | 26.37 | 8 |
| ATOM | 9956 | N | HIS | C | 843 | 31.393 | 96.047 | 39.389 | 1.00 | 19.36 | 7 |
| ATOM | 9957 | CA | HIS | C | 843 | 32.021 | 96.982 | 40.296 | 1.00 | 18.78 | 6 |
| ATOM | 9958 | CB | HIS | C | 843 | 31.155 | 97.173 | 41.514 | 1.00 | 18.72 | 6 |
| ATOM | 9959 | CG | HIS | C | 843 | 29.806 | 97.700 | 41.179 | 1.00 | 18.72 | 6 |
| ATOM | 9960 | CD2 | HIS | C | 843 | 28.572 | 97.195 | 41.390 | 1.00 | 19.21 | 6 |
| ATOM | 9961 | ND1 | HIS | C | 843 | 29.624 | 98.880 | 40.496 | 1.00 | 19.38 | 7 |
| ATOM | 9962 | CE1 | HIS | C | 843 | 28.335 | 99.082 | 40.301 | 1.00 | 19.92 | 6 |
| ATOM | 9963 | NE2 | HIS | C | 843 | 27.674 | 98.072 | 40.835 | 1.00 | 20.05 | 7 |
| ATOM | 9964 | C | HIS | C | 843 | 33.391 | 96.510 | 40.698 | 1.00 | 19.82 | 6 |
| ATOM | 9965 | O | HIS | C | 843 | 33.963 | 96.997 | 41.661 | 1.00 | 20.83 | 8 |
| ATOM | 9966 | N | GLY | C | 844 | 33.921 | 95.557 | 39.947 | 1.00 | 25.94 | 7 |
| ATOM | 9967 | CA | GLY | C | 844 | 35.243 | 95.051 | 40.242 | 1.00 | 25.22 | 6 |
| ATOM | 9968 | C | GLY | C | 844 | 35.308 | 94.143 | 41.446 | 1.00 | 25.40 | 6 |
| ATOM | 9969 | O | GLY | C | 844 | 36.258 | 94.234 | 42.219 | 1.00 | 26.35 | 8 |
| ATOM | 9970 | N | ASN | C | 845 | 34.313 | 93.275 | 41.620 | 1.00 | 22.56 | 7 |
| ATOM | 9971 | CA | ASN | C | 845 | 34.328 | 92.345 | 42.745 | 1.00 | 22.56 | 6 |
| ATOM | 9972 | CB | ASN | C | 845 | 32.914 | 92.095 | 43.267 | 1.00 | 42.57 | 6 |
| ATOM | 9973 | CG | ASN | C | 845 | 32.909 | 91.366 | 44.604 | 1.00 | 43.63 | 6 |
| ATOM | 9974 | OD1 | ASN | C | 845 | 33.444 | 90.267 | 44.727 | 1.00 | 42.96 | 8 |
| ATOM | 9975 | ND2 | ASN | C | 845 | 32.306 | 91.983 | 45.615 | 1.00 | 43.19 | 7 |
| ATOM | 9976 | C | ASN | C | 845 | 34.943 | 91.013 | 42.312 | 1.00 | 22.81 | 6 |
| ATOM | 9977 | O | ASN | C | 845 | 34.487 | 89.958 | 42.713 | 1.00 | 22.56 | 8 |
| ATOM | 9978 | N | LYS | C | 846 | 35.989 | 91.074 | 41.497 | 1.00 | 14.03 | 7 |
| ATOM | 9979 | CA | LYS | C | 846 | 36.652 | 89.882 | 40.995 | 1.00 | 13.87 | 6 |
| ATOM | 9980 | CB | LYS | C | 846 | 37.830 | 90.263 | 40.102 | 1.00 | 28.49 | 6 |
| ATOM | 9981 | CG | LYS | C | 846 | 38.811 | 91.249 | 40.704 | 1.00 | 31.68 | 6 |
| ATOM | 9982 | CD | LYS | C | 846 | 40.003 | 90.554 | 41.335 | 1.00 | 34.63 | 6 |
| ATOM | 9983 | CE | LYS | C | 846 | 41.150 | 91.540 | 41.528 | 1.00 | 35.89 | 6 |
| ATOM | 9984 | NZ | LYS | C | 846 | 41.629 | 92.107 | 40.224 | 1.00 | 34.76 | 7 |
| ATOM | 9985 | C | LYS | C | 846 | 37.135 | 88.932 | 42.048 | 1.00 | 13.87 | 6 |
| ATOM | 9986 | O | LYS | C | 846 | 37.985 | 89.281 | 42.850 | 1.00 | 13.87 | 8 |
| ATOM | 9987 | N | GLY | C | 847 | 36.588 | 87.722 | 42.017 | 1.00 | 27.87 | 7 |
| ATOM | 9988 | CA | GLY | C | 847 | 36.951 | 86.676 | 42.956 | 1.00 | 29.18 | 6 |
| ATOM | 9989 | C | GLY | C | 847 | 36.860 | 85.351 | 42.227 | 1.00 | 30.07 | 6 |
| ATOM | 9990 | O | GLY | C | 847 | 36.105 | 85.240 | 41.259 | 1.00 | 33.23 | 8 |
| ATOM | 9991 | N | VAL | C | 848 | 37.614 | 84.348 | 42.673 | 1.00 | 17.28 | 7 |
| ATOM | 9992 | CA | VAL | C | 848 | 37.610 | 83.027 | 42.031 | 1.00 | 16.07 | 6 |
| ATOM | 9993 | CB | VAL | C | 848 | 39.046 | 82.449 | 42.007 | 1.00 | 15.08 | 6 |
| ATOM | 9994 | CG1 | VAL | C | 848 | 39.440 | 81.913 | 43.381 | 1.00 | 14.66 | 6 |
| ATOM | 9995 | CG2 | VAL | C | 848 | 39.146 | 81.393 | 40.962 | 1.00 | 14.79 | 6 |
| ATOM | 9996 | C | VAL | C | 848 | 36.657 | 82.042 | 42.731 | 1.00 | 15.51 | 6 |
| ATOM | 9997 | O | VAL | C | 848 | 36.561 | 82.034 | 43.957 | 1.00 | 15.51 | 8 |
| ATOM | 9998 | N | VAL | C | 849 | 35.949 | 81.211 | 41.969 | 1.00 | 34.35 | 7 |
| ATOM | 9999 | CA | VAL | C | 849 | 35.014 | 80.278 | 42.602 | 1.00 | 35.20 | 6 |
| ATOM | 10000 | CB | VAL | C | 849 | 34.169 | 79.467 | 41.599 | 1.00 | 15.91 | 6 |
| ATOM | 10001 | CG1 | VAL | C | 849 | 33.191 | 78.610 | 42.375 | 1.00 | 15.91 | 6 |
| ATOM | 10002 | CG2 | VAL | C | 849 | 33.424 | 80.376 | 40.629 | 1.00 | 15.91 | 6 |
| ATOM | 10003 | C | VAL | C | 849 | 35.706 | 79.257 | 43.484 | 1.00 | 36.06 | 6 |
| ATOM | 10004 | O | VAL | C | 849 | 36.728 | 78.678 | 43.111 | 1.00 | 36.56 | 8 |
| ATOM | 10005 | N | ALA | C | 850 | 35.130 | 79.030 | 44.656 | 1.00 | 13.87 | 7 |
| ATOM | 10006 | CA | ALA | C | 850 | 35.682 | 78.075 | 45.598 | 1.00 | 13.87 | 6 |
| ATOM | 10007 | CB | ALA | C | 850 | 35.644 | 78.637 | 47.005 | 1.00 | 30.43 | 6 |
| ATOM | 10008 | C | ALA | C | 850 | 34.888 | 76.792 | 45.528 | 1.00 | 13.87 | 6 |
| ATOM | 10009 | O | ALA | C | 850 | 35.348 | 75.807 | 44.980 | 1.00 | 13.87 | 8 |
| ATOM | 10010 | N | LYS | C | 851 | 33.682 | 76.803 | 46.072 | 1.00 | 15.04 | 7 |
| ATOM | 10011 | CA | LYS | C | 851 | 32.852 | 75.615 | 46.058 | 1.00 | 16.33 | 6 |
| ATOM | 10012 | CB | LYS | C | 851 | 32.548 | 75.177 | 47.483 | 1.00 | 47.89 | 6 |
| ATOM | 10013 | CG | LYS | C | 851 | 33.670 | 74.453 | 48.154 | 1.00 | 51.90 | 6 |
| ATOM | 10014 | CD | LYS | C | 851 | 33.103 | 73.697 | 49.331 | 1.00 | 55.82 | 6 |
| ATOM | 10015 | CE | LYS | C | 851 | 34.049 | 72.604 | 49.804 | 1.00 | 58.16 | 6 |
| ATOM | 10016 | NZ | LYS | C | 851 | 33.488 | 71.855 | 50.970 | 1.00 | 61.84 | 7 |
| ATOM | 10017 | C | LYS | C | 851 | 31.545 | 75.811 | 45.314 | 1.00 | 17.23 | 6 |
| ATOM | 10018 | O | LYS | C | 851 | 31.112 | 76.932 | 45.075 | 1.00 | 16.53 | 8 |
| ATOM | 10019 | N | ILE | C | 852 | 30.927 | 74.705 | 44.930 | 1.00 | 30.21 | 7 |
| ATOM | 10020 | CA | ILE | C | 852 | 29.638 | 74.742 | 44.269 | 1.00 | 31.21 | 6 |
| ATOM | 10021 | CB | ILE | C | 852 | 29.695 | 74.259 | 42.809 | 1.00 | 45.44 | 6 |
| ATOM | 10022 | CG2 | ILE | C | 852 | 30.195 | 75.359 | 41.907 | 1.00 | 46.68 | 6 |
| ATOM | 10023 | CG1 | ILE | C | 852 | 30.589 | 73.025 | 42.702 | 1.00 | 47.88 | 6 |
| ATOM | 10024 | CD1 | ILE | C | 852 | 32.054 | 73.276 | 43.035 | 1.00 | 47.76 | 6 |
| ATOM | 10025 | C | ILE | C | 852 | 28.839 | 73.756 | 45.091 | 1.00 | 31.45 | 6 |
| ATOM | 10026 | O | ILE | C | 852 | 29.029 | 72.554 | 44.981 | 1.00 | 33.97 | 8 |
| ATOM | 10027 | N | LEU | C | 853 | 27.974 | 74.277 | 45.950 | 1.00 | 32.11 | 7 |
| ATOM | 10028 | CA | LEU | C | 853 | 27.147 | 73.456 | 46.818 | 1.00 | 32.54 | 6 |
| ATOM | 10029 | CB | LEU | C | 853 | 26.733 | 74.256 | 48.053 | 1.00 | 33.03 | 6 |
| ATOM | 10030 | CG | LEU | C | 853 | 27.652 | 74.338 | 49.272 | 1.00 | 34.33 | 6 |
| ATOM | 10031 | CD1 | LEU | C | 853 | 29.102 | 74.497 | 48.862 | 1.00 | 33.26 | 6 |
| ATOM | 10032 | CD2 | LEU | C | 853 | 27.192 | 75.506 | 50.142 | 1.00 | 33.78 | 6 |
| ATOM | 10033 | C | LEU | C | 853 | 25.892 | 72.950 | 46.112 | 1.00 | 32.54 | 6 |
| ATOM | 10034 | O | LEU | C | 853 | 25.429 | 73.538 | 45.129 | 1.00 | 33.26 | 8 |
| ATOM | 10035 | N | PRO | C | 854 | 25.324 | 71.842 | 46.614 | 1.00 | 15.91 | 7 |
| ATOM | 10036 | CD | PRO | C | 854 | 25.876 | 71.064 | 47.741 | 1.00 | 21.01 | 6 |
| ATOM | 10037 | CA | PRO | C | 854 | 24.114 | 71.211 | 46.081 | 1.00 | 15.76 | 6 |
| ATOM | 10038 | CB | PRO | C | 854 | 24.149 | 69.845 | 46.730 | 1.00 | 21.46 | 6 |
| ATOM | 10039 | CG | PRO | C | 854 | 24.742 | 70.151 | 48.093 | 1.00 | 21.49 | 6 |
| ATOM | 10040 | C | PRO | C | 854 | 22.939 | 72.036 | 46.563 | 1.00 | 15.96 | 6 |
| ATOM | 10041 | O | PRO | C | 854 | 22.963 | 72.518 | 47.693 | 1.00 | 16.82 | 8 |
| ATOM | 10042 | N | VAL | C | 855 | 21.913 | 72.208 | 45.736 | 1.00 | 13.99 | 7 |
| ATOM | 10043 | CA | VAL | C | 855 | 20.772 | 73.019 | 46.158 | 1.00 | 14.16 | 6 |
| ATOM | 10044 | CB | VAL | C | 855 | 19.572 | 72.873 | 45.191 | 1.00 | 15.96 | 6 |
| ATOM | 10045 | CG1 | VAL | C | 855 | 20.021 | 73.048 | 43.759 | 1.00 | 15.79 | 6 |
| ATOM | 10046 | CG2 | VAL | C | 855 | 18.933 | 71.538 | 45.370 | 1.00 | 16.37 | 6 |
| ATOM | 10047 | C | VAL | C | 855 | 20.328 | 72.636 | 47.577 | 1.00 | 15.92 | 6 |
| ATOM | 10048 | O | VAL | C | 855 | 19.685 | 73.444 | 48.261 | 1.00 | 14.68 | 8 |
| ATOM | 10049 | N | GLU | C | 856 | 20.684 | 71.406 | 47.994 | 1.00 | 47.18 | 7 |
| ATOM | 10050 | CA | GLU | C | 856 | 20.367 | 70.852 | 49.326 | 1.00 | 49.10 | 6 |
| ATOM | 10051 | CB | GLU | C | 856 | 21.216 | 69.613 | 49.641 | 1.00 | 76.95 | 6 |
| ATOM | 10052 | CG | GLU | C | 856 | 21.317 | 68.541 | 48.575 | 1.00 | 79.55 | 6 |
| ATOM | 10053 | CD | GLU | C | 856 | 22.169 | 67.352 | 49.041 | 1.00 | 81.76 | 6 |
| ATOM | 10054 | OE1 | GLU | C | 856 | 21.821 | 66.728 | 50.067 | 1.00 | 82.70 | 8 |
| ATOM | 10055 | OE2 | GLU | C | 856 | 23.188 | 67.042 | 48.386 | 1.00 | 82.38 | 8 |
| ATOM | 10056 | C | GLU | C | 856 | 20.706 | 71.896 | 50.380 | 1.00 | 49.17 | 6 |
| ATOM | 10057 | O | GLU | C | 856 | 19.829 | 72.501 | 51.003 | 1.00 | 48.86 | 8 |
| ATOM | 10058 | N | ASP | C | 857 | 22.008 | 72.060 | 50.590 | 1.00 | 29.03 | 7 |
| ATOM | 10059 | CA | ASP | C | 857 | 22.543 | 73.043 | 51.510 | 1.00 | 30.39 | 6 |
| ATOM | 10060 | CB | ASP | C | 857 | 24.056 | 72.915 | 51.579 | 1.00 | 31.58 | 6 |
| ATOM | 10061 | CG | ASP | C | 857 | 24.514 | 71.558 | 52.070 | 1.00 | 32.45 | 6 |
| ATOM | 10062 | OD1 | ASP | C | 857 | 24.547 | 71.347 | 53.310 | 1.00 | 32.14 | 8 |
| ATOM | 10063 | OD2 | ASP | C | 857 | 24.847 | 70.707 | 51.206 | 1.00 | 32.10 | 8 |
| ATOM | 10064 | C | ASP | C | 857 | 22.193 | 74.366 | 50.834 | 1.00 | 32.77 | 6 |
| ATOM | 10065 | O | ASP | C | 857 | 21.254 | 74.422 | 50.031 | 1.00 | 33.91 | 8 |
| ATOM | 10066 | N | MET | C | 858 | 22.945 | 75.423 | 51.130 | 1.00 | 44.36 | 7 |
| ATOM | 10067 | CA | MET | C | 858 | 22.690 | 76.724 | 50.512 | 1.00 | 46.64 | 6 |
| ATOM | 10068 | CB | MET | C | 858 | 22.422 | 76.583 | 49.005 | 1.00 | 52.89 | 6 |
| ATOM | 10069 | CG | MET | C | 858 | 23.611 | 76.178 | 48.158 | 1.00 | 53.88 | 6 |
| ATOM | 10070 | SD | MET | C | 858 | 24.764 | 77.516 | 47.938 | 1.00 | 53.56 | 16 |
| ATOM | 10071 | CE | MET | C | 858 | 23.670 | 78.836 | 47.358 | 1.00 | 51.17 | 6 |
| ATOM | 10072 | C | MET | C | 858 | 21.515 | 77.479 | 51.103 | 1.00 | 47.41 | 6 |
| ATOM | 10073 | O | MET | C | 858 | 20.402 | 76.966 | 51.163 | 1.00 | 47.52 | 8 |
| ATOM | 10074 | N | PRO | C | 859 | 21.756 | 78.716 | 51.548 | 1.00 | 45.14 | 7 |
| ATOM | 10075 | CD | PRO | C | 859 | 23.102 | 79.260 | 51.784 | 1.00 | 35.63 | 6 |
| ATOM | 10076 | CA | PRO | C | 859 | 20.753 | 79.599 | 52.131 | 1.00 | 46.72 | 6 |
| ATOM | 10077 | CB | PRO | C | 859 | 21.414 | 80.952 | 51.997 | 1.00 | 36.25 | 6 |
| ATOM | 10078 | CG | PRO | C | 859 | 22.817 | 80.627 | 52.396 | 1.00 | 36.31 | 6 |
| ATOM | 10079 | C | PRO | C | 859 | 19.393 | 79.528 | 51.421 | 1.00 | 47.95 | 6 |
| ATOM | 10080 | O | PRO | C | 859 | 19.257 | 79.935 | 50.260 | 1.00 | 47.86 | 8 |
| ATOM | 10081 | N | HIS | C | 860 | 18.407 | 78.985 | 52.138 | 1.00 | 63.92 | 7 |
| ATOM | 10082 | CA | HIS | C | 860 | 17.036 | 78.822 | 51.659 | 1.00 | 63.67 | 6 |
| ATOM | 10083 | CB | HIS | C | 860 | 16.332 | 77.716 | 52.477 | 1.00 | 52.57 | 6 |
| ATOM | 10084 | CG | HIS | C | 860 | 16.073 | 76.455 | 51.709 | 1.00 | 54.47 | 6 |
| ATOM | 10085 | CD2 | HIS | C | 860 | 16.692 | 75.252 | 51.733 | 1.00 | 55.68 | 6 |
| ATOM | 10086 | ND1 | HIS | C | 860 | 15.086 | 76.352 | 50.752 | 1.00 | 55.79 | 7 |
| ATOM | 10087 | CE1 | HIS | C | 860 | 15.109 | 75.143 | 50.218 | 1.00 | 55.08 | 6 |
| ATOM | 10088 | NE2 | HIS | C | 860 | 16.076 | 74.454 | 50.796 | 1.00 | 55.90 | 7 |
| ATOM | 10089 | C | HIS | C | 860 | 16.322 | 80.148 | 51.877 | 1.00 | 62.95 | 6 |
| ATOM | 10090 | O | HIS | C | 860 | 16.423 | 80.720 | 52.953 | 1.00 | 63.85 | 8 |
| ATOM | 10091 | N | LEU | C | 861 | 15.638 | 80.670 | 50.869 | 1.00 | 65.05 | 7 |
| ATOM | 10092 | CA | LEU | C | 861 | 14.901 | 81.910 | 51.091 | 1.00 | 65.21 | 6 |
| ATOM | 10093 | CB | LEU | C | 861 | 14.490 | 82.567 | 49.771 | 1.00 | 25.82 | 6 |
| ATOM | 10094 | CG | LEU | C | 861 | 14.298 | 84.089 | 49.799 | 1.00 | 24.82 | 6 |
| ATOM | 10095 | CD1 | LEU | C | 861 | 15.665 | 84.764 | 49.811 | 1.00 | 23.70 | 6 |
| ATOM | 10096 | CD2 | LEU | C | 861 | 13.492 | 84.550 | 48.600 | 1.00 | 23.90 | 6 |
| ATOM | 10097 | C | LEU | C | 861 | 13.680 | 81.331 | 51.795 | 1.00 | 66.71 | 6 |
| ATOM | 10098 | O | LEU | C | 861 | 13.516 | 80.110 | 51.809 | 1.00 | 68.75 | 8 |
| ATOM | 10099 | N | PRO | C | 862 | 12.803 | 82.170 | 52.375 | 1.00 | 74.55 | 7 |
| ATOM | 10100 | CD | PRO | C | 862 | 12.667 | 83.631 | 52.284 | 1.00 | 91.58 | 6 |
| ATOM | 10101 | CA | PRO | C | 862 | 11.638 | 81.592 | 53.053 | 1.00 | 72.97 | 6 |
| ATOM | 10102 | CB | PRO | C | 862 | 10.854 | 82.823 | 53.489 | 1.00 | 90.68 | 6 |
| ATOM | 10103 | CG | PRO | C | 862 | 11.177 | 83.808 | 52.414 | 1.00 | 92.13 | 6 |
| ATOM | 10104 | C | PRO | C | 862 | 10.802 | 80.634 | 52.207 | 1.00 | 71.31 | 6 |
| ATOM | 10105 | O | PRO | C | 862 | 10.680 | 79.451 | 52.537 | 1.00 | 71.81 | 8 |
| ATOM | 10106 | N | ASP | C | 863 | 10.238 | 81.137 | 51.114 | 1.00 | 34.21 | 7 |
| ATOM | 10107 | CA | ASP | C | 863 | 9.403 | 80.312 | 50.252 | 1.00 | 32.54 | 6 |
| ATOM | 10108 | CB | ASP | C | 863 | 8.863 | 81.146 | 49.096 | 1.00 | 51.46 | 6 |
| ATOM | 10109 | CG | ASP | C | 863 | 9.958 | 81.642 | 48.191 | 1.00 | 52.55 | 6 |
| ATOM | 10110 | OD1 | ASP | C | 863 | 9.676 | 82.477 | 47.305 | 1.00 | 52.96 | 8 |
| ATOM | 10111 | OD2 | ASP | C | 863 | 11.109 | 81.188 | 48.365 | 1.00 | 53.95 | 8 |
| ATOM | 10112 | C | ASP | C | 863 | 10.133 | 79.090 | 49.707 | 1.00 | 30.63 | 6 |
| ATOM | 10113 | O | ASP | C | 863 | 9.537 | 78.254 | 49.037 | 1.00 | 29.45 | 8 |
| ATOM | 10114 | N | GLY | C | 864 | 11.426 | 78.984 | 49.975 | 1.00 | 66.70 | 7 |
| ATOM | 10115 | CA | GLY | C | 864 | 12.153 | 77.824 | 49.497 | 1.00 | 65.94 | 6 |
| ATOM | 10116 | C | GLY | C | 864 | 13.085 | 78.016 | 48.317 | 1.00 | 64.47 | 6 |
| ATOM | 10117 | O | GLY | C | 864 | 13.931 | 77.151 | 48.051 | 1.00 | 65.00 | 8 |
| ATOM | 10118 | N | THR | C | 865 | 12.937 | 79.117 | 47.587 | 1.00 | 24.19 | 7 |
| ATOM | 10119 | CA | THR | C | 865 | 13.830 | 79.342 | 46.460 | 1.00 | 22.01 | 6 |
| ATOM | 10120 | CB | THR | C | 865 | 13.563 | 80.693 | 45.730 | 1.00 | 25.34 | 6 |
| ATOM | 10121 | OG1 | THR | C | 865 | 13.624 | 81.776 | 46.668 | 1.00 | 24.62 | 8 |
| ATOM | 10122 | CG2 | THR | C | 865 | 12.207 | 80.675 | 45.029 | 1.00 | 25.49 | 6 |
| ATOM | 10123 | C | THR | C | 865 | 15.213 | 79.381 | 47.079 | 1.00 | 20.25 | 6 |
| ATOM | 10124 | O | THR | C | 865 | 15.359 | 79.776 | 48.234 | 1.00 | 20.27 | 8 |
| ATOM | 10125 | N | PRO | C | 866 | 16.239 | 78.929 | 46.341 | 1.00 | 19.38 | 7 |
| ATOM | 10126 | CD | PRO | C | 866 | 16.176 | 78.105 | 45.118 | 1.00 | 30.86 | 6 |
| ATOM | 10127 | CA | PRO | C | 866 | 17.602 | 78.938 | 46.865 | 1.00 | 17.70 | 6 |
| ATOM | 10128 | CB | PRO | C | 866 | 18.203 | 77.699 | 46.240 | 1.00 | 29.62 | 6 |
| ATOM | 10129 | CG | PRO | C | 866 | 17.659 | 77.798 | 44.838 | 1.00 | 29.65 | 6 |
| ATOM | 10130 | C | PRO | C | 866 | 18.298 | 80.173 | 46.351 | 1.00 | 16.75 | 6 |
| ATOM | 10131 | O | PRO | C | 866 | 17.875 | 80.759 | 45.347 | 1.00 | 15.08 | 8 |
| ATOM | 10132 | N | VAL | C | 867 | 19.364 | 80.571 | 47.027 | 1.00 | 24.38 | 7 |
| ATOM | 10133 | CA | VAL | C | 867 | 20.107 | 81.718 | 46.555 | 1.00 | 24.88 | 6 |
| ATOM | 10134 | CB | VAL | C | 867 | 20.968 | 82.302 | 47.672 | 1.00 | 40.48 | 6 |
| ATOM | 10135 | CG1 | VAL | C | 867 | 21.400 | 83.712 | 47.311 | 1.00 | 40.84 | 6 |
| ATOM | 10136 | CG2 | VAL | C | 867 | 20.187 | 82.301 | 48.957 | 1.00 | 39.45 | 6 |
| ATOM | 10137 | C | VAL | C | 867 | 20.978 | 81.152 | 45.420 | 1.00 | 25.01 | 6 |
| ATOM | 10138 | O | VAL | C | 867 | 20.871 | 79.969 | 45.099 | 1.00 | 24.57 | 8 |
| ATOM | 10139 | N | ASP | C | 868 | 21.813 | 81.969 | 44.792 | 1.00 | 25.79 | 7 |
| ATOM | 10140 | CA | ASP | C | 868 | 22.639 | 81.447 | 43.717 | 1.00 | 28.08 | 6 |
| ATOM | 10141 | CB | ASP | C | 868 | 22.328 | 82.177 | 42.407 | 1.00 | 45.92 | 6 |
| ATOM | 10142 | CG | ASP | C | 868 | 20.984 | 81.759 | 41.807 | 1.00 | 50.99 | 6 |
| ATOM | 10143 | OD1 | ASP | C | 868 | 20.740 | 80.539 | 41.679 | 1.00 | 54.02 | 8 |
| ATOM | 10144 | OD2 | ASP | C | 868 | 20.175 | 82.641 | 41.445 | 1.00 | 51.57 | 8 |
| ATOM | 10145 | C | ASP | C | 868 | 24.127 | 81.522 | 44.041 | 1.00 | 27.89 | 6 |
| ATOM | 10146 | O | ASP | C | 868 | 24.825 | 80.505 | 44.014 | 1.00 | 29.18 | 8 |
| ATOM | 10147 | N | VAL | C | 869 | 24.610 | 82.722 | 44.354 | 1.00 | 13.87 | 7 |
| ATOM | 10148 | CA | VAL | C | 869 | 26.009 | 82.922 | 44.691 | 1.00 | 13.87 | 6 |
| ATOM | 10149 | CB | VAL | C | 869 | 26.691 | 83.807 | 43.643 | 1.00 | 13.87 | 6 |
| ATOM | 10150 | CG1 | VAL | C | 869 | 25.818 | 84.943 | 43.268 | 1.00 | 13.87 | 6 |
| ATOM | 10151 | CG2 | VAL | C | 869 | 27.936 | 84.339 | 44.177 | 1.00 | 13.87 | 6 |
| ATOM | 10152 | C | VAL | C | 869 | 26.119 | 83.539 | 46.080 | 1.00 | 13.87 | 6 |
| ATOM | 10153 | O | VAL | C | 869 | 25.347 | 84.412 | 46.428 | 1.00 | 13.87 | 8 |
| ATOM | 10154 | N | ILE | C | 870 | 27.062 | 83.059 | 46.883 | 1.00 | 20.02 | 7 |
| ATOM | 10155 | CA | ILE | C | 870 | 27.264 | 83.566 | 48.237 | 1.00 | 19.94 | 6 |
| ATOM | 10156 | CB | ILE | C | 870 | 27.458 | 82.415 | 49.248 | 1.00 | 16.17 | 6 |
| ATOM | 10157 | CG2 | ILE | C | 870 | 27.485 | 82.960 | 50.665 | 1.00 | 15.37 | 6 |
| ATOM | 10158 | CG1 | ILE | C | 870 | 26.313 | 81.415 | 49.139 | 1.00 | 15.26 | 6 |
| ATOM | 10159 | CD1 | ILE | C | 870 | 24.961 | 82.086 | 49.153 | 1.00 | 17.33 | 6 |
| ATOM | 10160 | C | ILE | C | 870 | 28.480 | 84.484 | 48.333 | 1.00 | 21.12 | 6 |
| ATOM | 10161 | O | ILE | C | 870 | 29.466 | 84.149 | 48.987 | 1.00 | 19.61 | 8 |
| ATOM | 10162 | N | LEU | C | 871 | 28.396 | 85.634 | 47.666 | 1.00 | 29.23 | 7 |
| ATOM | 10163 | CA | LEU | C | 871 | 29.450 | 86.652 | 47.649 | 1.00 | 30.67 | 6 |
| ATOM | 10164 | CB | LEU | C | 871 | 28.987 | 87.862 | 46.831 | 1.00 | 25.22 | 6 |
| ATOM | 10165 | CG | LEU | C | 871 | 29.698 | 88.283 | 45.537 | 1.00 | 27.31 | 6 |
| ATOM | 10166 | CD1 | LEU | C | 871 | 29.835 | 87.116 | 44.576 | 1.00 | 26.76 | 6 |
| ATOM | 10167 | CD2 | LEU | C | 871 | 28.911 | 89.422 | 44.894 | 1.00 | 26.18 | 6 |
| ATOM | 10168 | C | LEU | C | 871 | 29.755 | 87.102 | 49.078 | 1.00 | 31.91 | 6 |
| ATOM | 10169 | O | LEU | C | 871 | 28.861 | 87.514 | 49.816 | 1.00 | 32.07 | 8 |
| ATOM | 10170 | N | ASN | C | 872 | 31.024 | 87.033 | 49.461 | 1.00 | 63.95 | 7 |
| ATOM | 10171 | CA | ASN | C | 872 | 31.436 | 87.419 | 50.803 | 1.00 | 65.14 | 6 |
| ATOM | 10172 | CB | ASN | C | 872 | 32.814 | 86.861 | 51.117 | 1.00 | 62.52 | 6 |
| ATOM | 10173 | CG | ASN | C | 872 | 33.335 | 87.352 | 52.439 | 1.00 | 63.43 | 6 |
| ATOM | 10174 | OD1 | ASN | C | 872 | 32.982 | 86.825 | 53.488 | 1.00 | 62.80 | 8 |
| ATOM | 10175 | ND2 | ASN | C | 872 | 34.165 | 88.384 | 52.400 | 1.00 | 65.14 | 7 |
| ATOM | 10176 | C | ASN | C | 872 | 31.487 | 88.923 | 50.981 | 1.00 | 65.36 | 6 |
| ATOM | 10177 | O | ASN | C | 872 | 31.863 | 89.651 | 50.065 | 1.00 | 65.44 | 8 |
| ATOM | 10178 | N | PRO | C | 873 | 31.115 | 89.408 | 52.175 | 1.00 | 24.18 | 7 |
| ATOM | 10179 | CD | PRO | C | 873 | 30.480 | 88.651 | 53.270 | 1.00 | 49.41 | 6 |
| ATOM | 10180 | CA | PRO | C | 873 | 31.124 | 90.837 | 52.474 | 1.00 | 22.58 | 6 |
| ATOM | 10181 | CB | PRO | C | 873 | 30.085 | 90.960 | 53.573 | 1.00 | 47.18 | 6 |
| ATOM | 10182 | CG | PRO | C | 873 | 30.298 | 89.707 | 54.349 | 1.00 | 49.17 | 6 |
| ATOM | 10183 | C | PRO | C | 873 | 32.501 | 91.226 | 52.952 | 1.00 | 21.55 | 6 |
| ATOM | 10184 | O | PRO | C | 873 | 33.189 | 92.005 | 52.322 | 1.00 | 21.51 | 8 |
| ATOM | 10185 | N | LEU | C | 874 | 32.905 | 90.646 | 54.066 | 1.00 | 22.64 | 7 |
| ATOM | 10186 | CA | LEU | C | 874 | 34.197 | 90.935 | 54.660 | 1.00 | 24.20 | 6 |
| ATOM | 10187 | CB | LEU | C | 874 | 34.802 | 89.675 | 55.245 | 1.00 | 19.69 | 6 |
| ATOM | 10188 | CG | LEU | C | 874 | 35.431 | 89.954 | 56.589 | 1.00 | 17.49 | 6 |
| ATOM | 10189 | CD1 | LEU | C | 874 | 34.488 | 90.845 | 57.436 | 1.00 | 19.96 | 6 |
| ATOM | 10190 | CD2 | LEU | C | 874 | 35.711 | 88.636 | 57.248 | 1.00 | 15.59 | 6 |
| ATOM | 10191 | C | LEU | C | 874 | 35.171 | 91.560 | 53.694 | 1.00 | 26.40 | 6 |
| ATOM | 10192 | O | LEU | C | 874 | 35.351 | 91.094 | 52.554 | 1.00 | 24.93 | 8 |
| ATOM | 10193 | N | GLY | C | 875 | 35.820 | 92.614 | 54.167 | 1.00 | 35.44 | 7 |
| ATOM | 10194 | CA | GLY | C | 875 | 36.736 | 93.329 | 53.314 | 1.00 | 39.71 | 6 |
| ATOM | 10195 | C | GLY | C | 875 | 35.945 | 93.793 | 52.098 | 1.00 | 42.49 | 6 |
| ATOM | 10196 | O | GLY | C | 875 | 35.373 | 94.890 | 52.082 | 1.00 | 41.40 | 8 |
| ATOM | 10197 | N | VAL | C | 876 | 35.889 | 92.921 | 51.096 | 1.00 | 88.65 | 7 |
| ATOM | 10198 | CA | VAL | C | 876 | 35.203 | 93.188 | 49.846 | 1.00 | 90.76 | 6 |
| ATOM | 10199 | CB | VAL | C | 876 | 34.167 | 92.101 | 49.540 | 1.00 | 138.75 | 6 |
| ATOM | 10200 | CG1 | VAL | C | 876 | 33.611 | 92.305 | 48.141 | 1.00 | 138.36 | 6 |
| ATOM | 10201 | CG2 | VAL | C | 876 | 34.803 | 90.733 | 49.678 | 1.00 | 140.60 | 6 |
| ATOM | 10202 | C | VAL | C | 876 | 34.549 | 94.564 | 49.756 | 1.00 | 90.90 | 6 |
| ATOM | 10203 | O | VAL | C | 876 | 35.105 | 95.459 | 49.119 | 1.00 | 92.43 | 8 |
| ATOM | 10204 | N | PRO | C | 877 | 33.374 | 94.773 | 50.392 | 1.00 | 41.75 | 7 |
| ATOM | 10205 | CD | PRO | C | 877 | 32.308 | 93.935 | 50.975 | 1.00 | 30.43 | 6 |
| ATOM | 10206 | CA | PRO | C | 877 | 32.895 | 96.143 | 50.194 | 1.00 | 40.40 | 6 |
| ATOM | 10207 | CB | PRO | C | 877 | 31.393 | 96.032 | 50.524 | 1.00 | 28.90 | 6 |
| ATOM | 10208 | CG | PRO | C | 877 | 31.339 | 94.945 | 51.520 | 1.00 | 30.32 | 6 |
| ATOM | 10209 | C | PRO | C | 877 | 33.665 | 97.161 | 51.033 | 1.00 | 38.59 | 6 |
| ATOM | 10210 | O | PRO | C | 877 | 34.267 | 98.086 | 50.502 | 1.00 | 38.67 | 8 |
| ATOM | 10211 | N | SER | C | 878 | 33.660 | 96.965 | 52.340 | 1.00 | 16.22 | 7 |
| ATOM | 10212 | CA | SER | C | 878 | 34.359 | 97.848 | 53.251 | 1.00 | 15.21 | 6 |
| ATOM | 10213 | CB | SER | C | 878 | 34.472 | 97.149 | 54.578 | 1.00 | 28.46 | 6 |
| ATOM | 10214 | OG | SER | C | 878 | 34.790 | 95.801 | 54.305 | 1.00 | 25.61 | 8 |
| ATOM | 10215 | C | SER | C | 878 | 35.750 | 98.168 | 52.719 | 1.00 | 15.74 | 6 |
| ATOM | 10216 | O | SER | C | 878 | 36.062 | 99.322 | 52.438 | 1.00 | 14.86 | 8 |
| ATOM | 10217 | N | ARG | C | 879 | 36.581 | 97.136 | 52.586 | 1.00 | 46.60 | 7 |
| ATOM | 10218 | CA | ARG | C | 879 | 37.943 | 97.292 | 52.078 | 1.00 | 47.67 | 6 |
| ATOM | 10219 | CB | ARG | C | 879 | 38.572 | 95.926 | 51.784 | 1.00 | 65.31 | 6 |
| ATOM | 10220 | CG | ARG | C | 879 | 39.124 | 95.170 | 52.990 | 1.00 | 69.39 | 6 |
| ATOM | 10221 | CD | ARG | C | 879 | 39.713 | 93.827 | 52.557 | 1.00 | 71.49 | 6 |
| ATOM | 10222 | NE | ARG | C | 879 | 40.361 | 93.109 | 53.653 | 1.00 | 75.91 | 7 |
| ATOM | 10223 | CZ | ARG | C | 879 | 40.935 | 91.911 | 53.526 | 1.00 | 78.34 | 6 |
| ATOM | 10224 | NH1 | ARG | C | 879 | 40.936 | 91.299 | 52.345 | 1.00 | 79.75 | 7 |
| ATOM | 10225 | NH2 | ARG | C | 879 | 41.510 | 91.321 | 54.573 | 1.00 | 77.37 | 7 |
| ATOM | 10226 | C | ARG | C | 879 | 37.987 | 98.123 | 50.804 | 1.00 | 46.80 | 6 |
| ATOM | 10227 | O | ARG | C | 879 | 38.732 | 99.094 | 50.728 | 1.00 | 47.93 | 8 |
| ATOM | 10228 | N | MET | C | 880 | 37.183 | 97.741 | 49.810 | 1.00 | 24.76 | 7 |
| ATOM | 10229 | CA | MET | C | 880 | 37.147 | 98.435 | 48.517 | 1.00 | 22.28 | 6 |
| ATOM | 10230 | CB | MET | C | 880 | 36.880 | 97.427 | 47.395 | 1.00 | 60.39 | 6 |
| ATOM | 10231 | CG | MET | C | 880 | 37.912 | 96.309 | 47.273 | 1.00 | 62.40 | 6 |
| ATOM | 10232 | SD | MET | C | 880 | 37.903 | 95.102 | 48.629 | 1.00 | 63.95 | 16 |
| ATOM | 10233 | CE | MET | C | 880 | 38.835 | 93.688 | 47.883 | 1.00 | 60.88 | 6 |
| ATOM | 10234 | C | MET | C | 880 | 36.145 | 99.588 | 48.375 | 1.00 | 20.22 | 6 |
| ATOM | 10235 | O | MET | C | 880 | 36.201 | 100.333 | 47.403 | 1.00 | 19.81 | 8 |
| ATOM | 10236 | N | ASN | C | 881 | 35.255 | 99.748 | 49.349 | 1.00 | 17.01 | 7 |
| ATOM | 10237 | CA | ASN | C | 881 | 34.230 | 100.790 | 49.308 | 1.00 | 14.46 | 6 |
| ATOM | 10238 | CB | ASN | C | 881 | 34.720 | 102.131 | 49.864 | 1.00 | 19.58 | 6 |
| ATOM | 10239 | CG | ASN | C | 881 | 36.210 | 102.274 | 49.838 | 1.00 | 20.04 | 6 |
| ATOM | 10240 | OD1 | ASN | C | 881 | 36.741 | 103.305 | 49.422 | 1.00 | 21.91 | 8 |
| ATOM | 10241 | ND2 | ASN | C | 881 | 36.904 | 101.257 | 50.315 | 1.00 | 20.68 | 7 |
| ATOM | 10242 | C | ASN | C | 881 | 33.692 | 101.002 | 47.920 | 1.00 | 13.87 | 6 |
| ATOM | 10243 | O | ASN | C | 881 | 34.295 | 101.684 | 47.116 | 1.00 | 13.87 | 8 |
| ATOM | 10244 | N | LEU | C | 882 | 32.524 | 100.420 | 47.670 | 1.00 | 51.63 | 7 |
| ATOM | 10245 | CA | LEU | C | 882 | 31.876 | 100.495 | 46.374 | 1.00 | 53.27 | 6 |
| ATOM | 10246 | CB | LEU | C | 882 | 32.490 | 99.424 | 45.468 | 1.00 | 19.17 | 6 |
| ATOM | 10247 | CG | LEU | C | 882 | 32.657 | 98.023 | 46.080 | 1.00 | 17.88 | 6 |
| ATOM | 10248 | CD1 | LEU | C | 882 | 33.303 | 97.111 | 45.059 | 1.00 | 18.68 | 6 |
| ATOM | 10249 | CD2 | LEU | C | 882 | 33.529 | 98.062 | 47.321 | 1.00 | 17.17 | 6 |
| ATOM | 10250 | C | LEU | C | 882 | 30.335 | 100.361 | 46.436 | 1.00 | 55.80 | 6 |
| ATOM | 10251 | O | LEU | C | 882 | 29.734 | 100.224 | 47.515 | 1.00 | 56.16 | 8 |
| ATOM | 10252 | N | GLY | C | 883 | 29.706 | 100.428 | 45.265 | 1.00 | 66.41 | 7 |
| ATOM | 10253 | CA | GLY | C | 883 | 28.259 | 100.328 | 45.178 | 1.00 | 68.46 | 6 |
| ATOM | 10254 | C | GLY | C | 883 | 27.731 | 99.081 | 44.479 | 1.00 | 71.24 | 6 |
| ATOM | 10255 | O | GLY | C | 883 | 26.817 | 99.171 | 43.653 | 1.00 | 73.20 | 8 |
| ATOM | 10256 | N | GLN | C | 884 | 28.310 | 97.921 | 44.799 | 1.00 | 62.65 | 7 |
| ATOM | 10257 | CA | GLN | C | 884 | 27.874 | 96.647 | 44.234 | 1.00 | 58.56 | 6 |
| ATOM | 10258 | CB | GLN | C | 884 | 28.962 | 95.574 | 44.378 | 1.00 | 21.91 | 6 |
| ATOM | 10259 | CG | GLN | C | 884 | 29.676 | 95.550 | 45.734 | 1.00 | 18.03 | 6 |
| ATOM | 10260 | CD | GLN | C | 884 | 30.662 | 94.400 | 45.858 | 1.00 | 15.22 | 6 |
| ATOM | 10261 | OE1 | GLN | C | 884 | 30.296 | 93.246 | 45.649 | 1.00 | 13.87 | 8 |
| ATOM | 10262 | NE2 | GLN | C | 884 | 31.914 | 94.708 | 46.203 | 1.00 | 13.87 | 7 |
| ATOM | 10263 | C | GLN | C | 884 | 26.639 | 96.229 | 44.996 | 1.00 | 57.37 | 6 |
| ATOM | 10264 | O | GLN | C | 884 | 26.055 | 95.188 | 44.733 | 1.00 | 58.54 | 8 |
| ATOM | 10265 | N | ILE | C | 885 | 26.257 | 97.059 | 45.959 | 1.00 | 69.69 | 7 |
| ATOM | 10266 | CA | ILE | C | 885 | 25.083 | 96.806 | 46.768 | 1.00 | 68.35 | 6 |
| ATOM | 10267 | CB | ILE | C | 885 | 25.244 | 97.392 | 48.181 | 1.00 | 13.87 | 6 |
| ATOM | 10268 | CG2 | ILE | C | 885 | 23.906 | 97.486 | 48.854 | 1.00 | 13.87 | 6 |
| ATOM | 10269 | CG1 | ILE | C | 885 | 26.111 | 96.480 | 49.032 | 1.00 | 13.87 | 6 |
| ATOM | 10270 | CD1 | ILE | C | 885 | 27.491 | 96.388 | 48.562 | 1.00 | 13.87 | 6 |
| ATOM | 10271 | C | ILE | C | 885 | 23.857 | 97.413 | 46.081 | 1.00 | 70.41 | 6 |
| ATOM | 10272 | O | ILE | C | 885 | 22.909 | 96.697 | 45.752 | 1.00 | 70.98 | 8 |
| ATOM | 10273 | N | LEU | C | 886 | 23.860 | 98.724 | 45.868 | 1.00 | 50.09 | 7 |
| ATOM | 10274 | CA | LEU | C | 886 | 22.731 | 99.338 | 45.187 | 1.00 | 50.40 | 6 |
| ATOM | 10275 | CB | LEU | C | 886 | 22.964 | 100.831 | 44.949 | 1.00 | 31.30 | 6 |
| ATOM | 10276 | CG | LEU | C | 886 | 23.251 | 101.847 | 46.053 | 1.00 | 29.77 | 6 |
| ATOM | 10277 | CD1 | LEU | C | 886 | 22.945 | 103.171 | 45.433 | 1.00 | 28.98 | 6 |
| ATOM | 10278 | CD2 | LEU | C | 886 | 22.402 | 101.678 | 47.315 | 1.00 | 29.77 | 6 |
| ATOM | 10279 | C | LEU | C | 886 | 22.642 | 98.622 | 43.840 | 1.00 | 50.97 | 6 |
| ATOM | 10280 | O | LEU | C | 886 | 23.254 | 99.035 | 42.858 | 1.00 | 51.31 | 8 |
| ATOM | 10281 | N | GLU | C | 887 | 21.887 | 97.536 | 43.813 | 1.00 | 73.13 | 7 |
| ATOM | 10282 | CA | GLU | C | 887 | 21.705 | 96.714 | 42.621 | 1.00 | 73.48 | 6 |
| ATOM | 10283 | CB | GLU | C | 887 | 23.031 | 96.444 | 41.890 | 1.00 | 45.79 | 6 |
| ATOM | 10284 | CG | GLU | C | 887 | 22.897 | 95.396 | 40.756 | 1.00 | 46.01 | 6 |
| ATOM | 10285 | CD | GLU | C | 887 | 24.226 | 94.969 | 40.135 | 1.00 | 45.65 | 6 |
| ATOM | 10286 | OE1 | GLU | C | 887 | 24.961 | 95.823 | 39.583 | 1.00 | 44.07 | 8 |
| ATOM | 10287 | OE2 | GLU | C | 887 | 24.525 | 93.759 | 40.199 | 1.00 | 46.39 | 8 |
| ATOM | 10288 | C | GLU | C | 887 | 21.210 | 95.418 | 43.194 | 1.00 | 72.66 | 6 |
| ATOM | 10289 | O | GLU | C | 887 | 20.327 | 94.772 | 42.652 | 1.00 | 73.29 | 8 |
| ATOM | 10290 | N | THR | C | 888 | 21.828 | 95.047 | 44.303 | 1.00 | 18.17 | 7 |
| ATOM | 10291 | CA | THR | C | 888 | 21.487 | 93.851 | 45.023 | 1.00 | 15.33 | 6 |
| ATOM | 10292 | CB | THR | C | 888 | 22.585 | 93.541 | 45.959 | 1.00 | 13.87 | 6 |
| ATOM | 10293 | OG1 | THR | C | 888 | 23.802 | 93.930 | 45.324 | 1.00 | 13.87 | 8 |
| ATOM | 10294 | CG2 | THR | C | 888 | 22.614 | 92.051 | 46.293 | 1.00 | 13.87 | 6 |
| ATOM | 10295 | C | THR | C | 888 | 20.234 | 94.213 | 45.792 | 1.00 | 16.41 | 6 |
| ATOM | 10296 | O | THR | C | 888 | 19.386 | 93.361 | 46.071 | 1.00 | 16.99 | 8 |
| ATOM | 10297 | N | HIS | C | 889 | 20.137 | 95.497 | 46.137 | 1.00 | 32.23 | 7 |
| ATOM | 10298 | CA | HIS | C | 889 | 18.979 | 96.033 | 46.831 | 1.00 | 32.75 | 6 |
| ATOM | 10299 | CB | HIS | C | 889 | 19.329 | 97.333 | 47.539 | 1.00 | 39.22 | 6 |
| ATOM | 10300 | CG | HIS | C | 889 | 20.145 | 97.137 | 48.777 | 1.00 | 39.27 | 6 |
| ATOM | 10301 | CD2 | HIS | C | 889 | 20.992 | 96.145 | 49.142 | 1.00 | 38.72 | 6 |
| ATOM | 10302 | ND1 | HIS | C | 889 | 20.135 | 98.035 | 49.821 | 1.00 | 40.02 | 7 |
| ATOM | 10303 | CE1 | HIS | C | 889 | 20.939 | 97.604 | 50.777 | 1.00 | 39.04 | 6 |
| ATOM | 10304 | NE2 | HIS | C | 889 | 21.474 | 96.459 | 50.391 | 1.00 | 38.17 | 7 |
| ATOM | 10305 | C | HIS | C | 889 | 17.952 | 96.281 | 45.740 | 1.00 | 33.38 | 6 |
| ATOM | 10306 | O | HIS | C | 889 | 16.850 | 95.732 | 45.788 | 1.00 | 34.33 | 8 |
| ATOM | 10307 | N | LEU | C | 890 | 18.296 | 97.091 | 44.739 | 1.00 | 18.78 | 7 |
| ATOM | 10308 | CA | LEU | C | 890 | 17.352 | 97.293 | 43.645 | 1.00 | 18.34 | 6 |
| ATOM | 10309 | CB | LEU | C | 890 | 17.837 | 98.355 | 42.667 | 1.00 | 13.87 | 6 |
| ATOM | 10310 | CG | LEU | C | 890 | 16.915 | 98.456 | 41.452 | 1.00 | 13.87 | 6 |
| ATOM | 10311 | CD1 | LEU | C | 890 | 15.643 | 99.165 | 41.840 | 1.00 | 13.87 | 6 |
| ATOM | 10312 | CD2 | LEU | C | 890 | 17.604 | 99.158 | 40.334 | 1.00 | 13.87 | 6 |
| ATOM | 10313 | C | LEU | C | 890 | 17.285 | 95.946 | 42.920 | 1.00 | 19.41 | 6 |
| ATOM | 10314 | O | LEU | C | 890 | 16.892 | 95.861 | 41.762 | 1.00 | 19.66 | 8 |
| ATOM | 10315 | N | GLY | C | 891 | 17.676 | 94.890 | 43.623 | 1.00 | 60.57 | 7 |
| ATOM | 10316 | CA | GLY | C | 891 | 17.682 | 93.560 | 43.042 | 1.00 | 63.51 | 6 |
| ATOM | 10317 | C | GLY | C | 891 | 16.382 | 92.814 | 43.186 | 1.00 | 64.40 | 6 |
| ATOM | 10318 | O | GLY | C | 891 | 15.733 | 92.497 | 42.190 | 1.00 | 65.49 | 8 |
| ATOM | 10319 | N | LEU | C | 892 | 16.001 | 92.510 | 44.420 | 1.00 | 34.08 | 7 |
| ATOM | 10320 | CA | LEU | C | 892 | 14.749 | 91.804 | 44.620 | 1.00 | 33.79 | 6 |
| ATOM | 10321 | CB | LEU | C | 892 | 14.633 | 91.282 | 46.049 | 1.00 | 28.52 | 6 |
| ATOM | 10322 | CG | LEU | C | 892 | 14.848 | 92.212 | 47.212 | 1.00 | 27.72 | 6 |
| ATOM | 10323 | CD1 | LEU | C | 892 | 15.482 | 91.388 | 48.297 | 1.00 | 27.36 | 6 |
| ATOM | 10324 | CD2 | LEU | C | 892 | 15.738 | 93.357 | 46.826 | 1.00 | 25.52 | 6 |
| ATOM | 10325 | C | LEU | C | 892 | 13.663 | 92.792 | 44.284 | 1.00 | 33.60 | 6 |
| ATOM | 10326 | O | LEU | C | 892 | 12.543 | 92.421 | 43.918 | 1.00 | 33.48 | 8 |
| ATOM | 10327 | N | ALA | C | 893 | 14.026 | 94.064 | 44.383 | 1.00 | 62.28 | 7 |
| ATOM | 10328 | CA | ALA | C | 893 | 13.115 | 95.140 | 44.039 | 1.00 | 63.02 | 6 |
| ATOM | 10329 | CB | ALA | C | 893 | 13.581 | 96.448 | 44.660 | 1.00 | 117.47 | 6 |
| ATOM | 10330 | C | ALA | C | 893 | 13.211 | 95.196 | 42.519 | 1.00 | 62.12 | 6 |
| ATOM | 10331 | O | ALA | C | 893 | 13.429 | 96.246 | 41.919 | 1.00 | 62.53 | 8 |
| ATOM | 10332 | N | GLY | C | 894 | 13.060 | 94.028 | 41.913 | 1.00 | 23.96 | 7 |
| ATOM | 10333 | CA | GLY | C | 894 | 13.148 | 93.915 | 40.479 | 1.00 | 22.93 | 6 |
| ATOM | 10334 | C | GLY | C | 894 | 13.160 | 92.432 | 40.213 | 1.00 | 22.79 | 6 |
| ATOM | 10335 | O | GLY | C | 894 | 13.377 | 91.959 | 39.095 | 1.00 | 23.57 | 8 |
| ATOM | 10336 | N | TYR | C | 895 | 12.957 | 91.678 | 41.275 | 1.00 | 25.96 | 7 |
| ATOM | 10337 | CA | TYR | C | 895 | 12.896 | 90.244 | 41.146 | 1.00 | 25.66 | 6 |
| ATOM | 10338 | CB | TYR | C | 895 | 13.561 | 89.614 | 42.356 | 1.00 | 23.90 | 6 |
| ATOM | 10339 | CG | TYR | C | 895 | 13.542 | 88.111 | 42.429 | 1.00 | 22.18 | 6 |
| ATOM | 10340 | CD1 | TYR | C | 895 | 13.895 | 87.331 | 41.338 | 1.00 | 21.01 | 6 |
| ATOM | 10341 | CE1 | TYR | C | 895 | 14.002 | 85.946 | 41.460 | 1.00 | 21.27 | 6 |
| ATOM | 10342 | CD2 | TYR | C | 895 | 13.283 | 87.471 | 43.642 | 1.00 | 21.87 | 6 |
| ATOM | 10343 | CE2 | TYR | C | 895 | 13.392 | 86.095 | 43.777 | 1.00 | 20.71 | 6 |
| ATOM | 10344 | CZ | TYR | C | 895 | 13.756 | 85.339 | 42.687 | 1.00 | 20.90 | 6 |
| ATOM | 10345 | OH | TYR | C | 895 | 13.916 | 83.983 | 42.839 | 1.00 | 21.62 | 8 |
| ATOM | 10346 | C | TYR | C | 895 | 11.389 | 90.020 | 41.147 | 1.00 | 26.84 | 6 |
| ATOM | 10347 | O | TYR | C | 895 | 10.783 | 89.748 | 40.097 | 1.00 | 26.90 | 8 |
| ATOM | 10348 | N | PHE | C | 896 | 10.785 | 90.204 | 42.321 | 1.00 | 34.06 | 7 |
| ATOM | 10349 | CA | PHE | C | 896 | 9.347 | 90.041 | 42.504 | 1.00 | 35.20 | 6 |
| ATOM | 10350 | CB | PHE | C | 896 | 9.009 | 90.241 | 43.968 | 1.00 | 27.57 | 6 |
| ATOM | 10351 | CG | PHE | C | 896 | 9.814 | 89.398 | 44.874 | 1.00 | 26.38 | 6 |
| ATOM | 10352 | CD1 | PHE | C | 896 | 11.089 | 89.785 | 45.240 | 1.00 | 25.96 | 6 |
| ATOM | 10353 | CD2 | PHE | C | 896 | 9.294 | 88.215 | 45.375 | 1.00 | 27.46 | 6 |
| ATOM | 10354 | CE1 | PHE | C | 896 | 11.843 | 89.002 | 46.104 | 1.00 | 27.11 | 6 |
| ATOM | 10355 | CE2 | PHE | C | 896 | 10.028 | 87.421 | 46.233 | 1.00 | 28.63 | 6 |
| ATOM | 10356 | CZ | PHE | C | 896 | 11.309 | 87.813 | 46.605 | 1.00 | 28.80 | 6 |
| ATOM | 10357 | C | PHE | C | 896 | 8.510 | 91.000 | 41.652 | 1.00 | 36.70 | 6 |
| ATOM | 10358 | O | PHE | C | 896 | 7.360 | 90.716 | 41.305 | 1.00 | 36.27 | 8 |
| ATOM | 10359 | N | LEU | C | 897 | 9.089 | 92.142 | 41.320 | 1.00 | 58.51 | 7 |
| ATOM | 10360 | CA | LEU | C | 897 | 8.384 | 93.114 | 40.514 | 1.00 | 60.85 | 6 |
| ATOM | 10361 | CB | LEU | C | 897 | 8.817 | 94.517 | 40.923 | 1.00 | 42.83 | 6 |
| ATOM | 10362 | CG | LEU | C | 897 | 7.724 | 95.579 | 41.013 | 1.00 | 42.87 | 6 |
| ATOM | 10363 | CD1 | LEU | C | 897 | 6.707 | 95.182 | 42.071 | 1.00 | 43.38 | 6 |
| ATOM | 10364 | CD2 | LEU | C | 897 | 8.354 | 96.907 | 41.369 | 1.00 | 43.44 | 6 |
| ATOM | 10365 | C | LEU | C | 897 | 8.671 | 92.884 | 39.025 | 1.00 | 61.83 | 6 |
| ATOM | 10366 | O | LEU | C | 897 | 8.295 | 93.706 | 38.189 | 1.00 | 62.46 | 8 |
| ATOM | 10367 | N | GLY | C | 898 | 9.324 | 91.763 | 38.704 | 1.00 | 38.99 | 7 |
| ATOM | 10368 | CA | GLY | C | 898 | 9.661 | 91.456 | 37.322 | 1.00 | 39.51 | 6 |
| ATOM | 10369 | C | GLY | C | 898 | 10.033 | 92.716 | 36.563 | 1.00 | 39.35 | 6 |
| ATOM | 10370 | O | GLY | C | 898 | 9.219 | 93.252 | 35.819 | 1.00 | 38.82 | 8 |
| ATOM | 10371 | N | GLN | C | 899 | 11.261 | 93.193 | 36.749 | 1.00 | 40.96 | 7 |
| ATOM | 10372 | CA | GLN | C | 899 | 11.712 | 94.416 | 36.097 | 1.00 | 42.62 | 6 |
| ATOM | 10373 | CB | GLN | C | 899 | 11.332 | 95.630 | 36.950 | 1.00 | 41.92 | 6 |
| ATOM | 10374 | CG | GLN | C | 899 | 9.883 | 96.045 | 36.921 | 1.00 | 46.22 | 6 |
| ATOM | 10375 | CD | GLN | C | 899 | 9.617 | 97.280 | 37.768 | 1.00 | 48.59 | 6 |
| ATOM | 10376 | OE1 | GLN | C | 899 | 9.692 | 97.234 | 38.992 | 1.00 | 49.74 | 8 |
| ATOM | 10377 | NE2 | GLN | C | 899 | 9.311 | 98.395 | 37.111 | 1.00 | 50.31 | 7 |
| ATOM | 10378 | C | GLN | C | 899 | 13.211 | 94.523 | 35.837 | 1.00 | 41.70 | 6 |
| ATOM | 10379 | O | GLN | C | 899 | 14.030 | 94.068 | 36.633 | 1.00 | 40.67 | 8 |
| ATOM | 10380 | N | ARG | C | 900 | 13.564 | 95.135 | 34.714 | 1.00 | 34.30 | 7 |
| ATOM | 10381 | CA | ARG | C | 900 | 14.963 | 95.406 | 34.410 | 1.00 | 34.45 | 6 |
| ATOM | 10382 | CB | ARG | C | 900 | 15.275 | 95.093 | 32.936 | 1.00 | 69.44 | 6 |
| ATOM | 10383 | CG | ARG | C | 900 | 15.347 | 93.590 | 32.616 | 1.00 | 72.22 | 6 |
| ATOM | 10384 | CD | ARG | C | 900 | 16.080 | 93.265 | 31.293 | 1.00 | 74.51 | 6 |
| ATOM | 10385 | NE | ARG | C | 900 | 15.222 | 93.302 | 30.102 | 1.00 | 77.19 | 7 |
| ATOM | 10386 | CZ | ARG | C | 900 | 15.629 | 92.999 | 28.867 | 1.00 | 79.00 | 6 |
| ATOM | 10387 | NH1 | ARG | C | 900 | 16.886 | 92.636 | 28.640 | 1.00 | 79.20 | 7 |
| ATOM | 10388 | NH2 | ARG | C | 900 | 14.779 | 93.043 | 27.850 | 1.00 | 80.28 | 7 |
| ATOM | 10389 | C | ARG | C | 900 | 15.064 | 96.927 | 34.729 | 1.00 | 32.73 | 6 |
| ATOM | 10390 | O | ARG | C | 900 | 14.042 | 97.616 | 34.797 | 1.00 | 31.77 | 8 |
| ATOM | 10391 | N | TYR | C | 901 | 16.256 | 97.467 | 34.959 | 1.00 | 28.86 | 7 |
| ATOM | 10392 | CA | TYR | C | 901 | 16.333 | 98.884 | 35.299 | 1.00 | 27.06 | 6 |
| ATOM | 10393 | CB | TYR | C | 901 | 16.461 | 99.057 | 36.816 | 1.00 | 51.17 | 6 |
| ATOM | 10394 | CG | TYR | C | 901 | 15.344 | 98.445 | 37.637 | 1.00 | 54.33 | 6 |
| ATOM | 10395 | CD1 | TYR | C | 901 | 15.087 | 97.082 | 37.587 | 1.00 | 56.21 | 6 |
| ATOM | 10396 | CE1 | TYR | C | 901 | 14.067 | 96.511 | 38.332 | 1.00 | 56.58 | 6 |
| ATOM | 10397 | CD2 | TYR | C | 901 | 14.546 | 99.232 | 38.469 | 1.00 | 54.81 | 6 |
| ATOM | 10398 | CE2 | TYR | C | 901 | 13.519 | 98.667 | 39.227 | 1.00 | 55.85 | 6 |
| ATOM | 10399 | CZ | TYR | C | 901 | 13.287 | 97.303 | 39.146 | 1.00 | 56.44 | 6 |
| ATOM | 10400 | OH | TYR | C | 901 | 12.266 | 96.719 | 39.850 | 1.00 | 57.80 | 8 |
| ATOM | 10401 | C | TYR | C | 901 | 17.463 | 99.650 | 34.627 | 1.00 | 25.11 | 6 |
| ATOM | 10402 | O | TYR | C | 901 | 18.541 | 99.111 | 34.388 | 1.00 | 23.97 | 8 |
| ATOM | 10403 | N | ILE | C | 902 | 17.194 | 100.922 | 34.337 | 1.00 | 36.72 | 7 |
| ATOM | 10404 | CA | ILE | C | 902 | 18.157 | 101.815 | 33.702 | 1.00 | 35.73 | 6 |
| ATOM | 10405 | CB | ILE | C | 902 | 17.614 | 102.405 | 32.381 | 1.00 | 35.77 | 6 |
| ATOM | 10406 | CG2 | ILE | C | 902 | 18.461 | 103.585 | 31.962 | 1.00 | 36.26 | 6 |
| ATOM | 10407 | CG1 | ILE | C | 902 | 17.681 | 101.365 | 31.266 | 1.00 | 37.30 | 6 |
| ATOM | 10408 | CD1 | ILE | C | 902 | 17.032 | 100.043 | 31.613 | 1.00 | 40.87 | 6 |
| ATOM | 10409 | C | ILE | C | 902 | 18.524 | 102.968 | 34.631 | 1.00 | 35.26 | 6 |
| ATOM | 10410 | O | ILE | C | 902 | 17.849 | 104.000 | 34.676 | 1.00 | 35.12 | 8 |
| ATOM | 10411 | N | SER | C | 903 | 19.607 | 102.772 | 35.372 | 1.00 | 31.96 | 7 |
| ATOM | 10412 | CA | SER | C | 903 | 20.119 | 103.754 | 36.311 | 1.00 | 30.93 | 6 |
| ATOM | 10413 | CB | SER | C | 903 | 20.228 | 103.127 | 37.698 | 1.00 | 34.74 | 6 |
| ATOM | 10414 | OG | SER | C | 903 | 21.452 | 103.468 | 38.322 | 1.00 | 35.86 | 8 |
| ATOM | 10415 | C | SER | C | 903 | 21.491 | 104.161 | 35.820 | 1.00 | 29.29 | 6 |
| ATOM | 10416 | O | SER | C | 903 | 22.489 | 103.590 | 36.226 | 1.00 | 28.82 | 8 |
| ATOM | 10417 | N | PRO | C | 904 | 21.552 | 105.151 | 34.922 | 1.00 | 60.94 | 7 |
| ATOM | 10418 | CD | PRO | C | 904 | 20.412 | 105.862 | 34.321 | 1.00 | 13.87 | 6 |
| ATOM | 10419 | CA | PRO | C | 904 | 22.822 | 105.630 | 34.366 | 1.00 | 60.49 | 6 |
| ATOM | 10420 | CB | PRO | C | 904 | 22.376 | 106.639 | 33.302 | 1.00 | 14.23 | 6 |
| ATOM | 10421 | CG | PRO | C | 904 | 21.065 | 107.114 | 33.816 | 1.00 | 14.25 | 6 |
| ATOM | 10422 | C | PRO | C | 904 | 23.842 | 106.206 | 35.352 | 1.00 | 59.70 | 6 |
| ATOM | 10423 | O | PRO | C | 904 | 23.516 | 106.510 | 36.505 | 1.00 | 60.46 | 8 |
| ATOM | 10424 | N | VAL | C | 905 | 25.077 | 106.340 | 34.863 | 1.00 | 15.62 | 7 |
| ATOM | 10425 | CA | VAL | C | 905 | 26.218 | 106.848 | 35.622 | 1.00 | 14.36 | 6 |
| ATOM | 10426 | CB | VAL | C | 905 | 27.500 | 106.820 | 34.790 | 1.00 | 29.83 | 6 |
| ATOM | 10427 | CG1 | VAL | C | 905 | 28.694 | 106.940 | 35.705 | 1.00 | 29.87 | 6 |
| ATOM | 10428 | CG2 | VAL | C | 905 | 27.565 | 105.557 | 33.962 | 1.00 | 31.57 | 6 |
| ATOM | 10429 | C | VAL | C | 905 | 26.055 | 108.269 | 36.119 | 1.00 | 13.92 | 6 |
| ATOM | 10430 | O | VAL | C | 905 | 26.104 | 109.228 | 35.344 | 1.00 | 13.87 | 8 |
| ATOM | 10431 | N | PHE | C | 906 | 25.879 | 108.382 | 37.431 | 1.00 | 30.04 | 7 |
| ATOM | 10432 | CA | PHE | C | 906 | 25.711 | 109.659 | 38.099 | 1.00 | 30.51 | 6 |
| ATOM | 10433 | CB | PHE | C | 906 | 26.810 | 110.618 | 37.657 | 1.00 | 19.82 | 6 |
| ATOM | 10434 | CG | PHE | C | 906 | 28.186 | 110.161 | 38.018 | 1.00 | 19.64 | 6 |
| ATOM | 10435 | CD1 | PHE | C | 906 | 29.235 | 110.307 | 37.127 | 1.00 | 18.44 | 6 |
| ATOM | 10436 | CD2 | PHE | C | 906 | 28.445 | 109.597 | 39.266 | 1.00 | 20.87 | 6 |
| ATOM | 10437 | CE1 | PHE | C | 906 | 30.525 | 109.901 | 37.471 | 1.00 | 18.44 | 6 |
| ATOM | 10438 | CE2 | PHE | C | 906 | 29.742 | 109.186 | 39.618 | 1.00 | 19.51 | 6 |
| ATOM | 10439 | CZ | PHE | C | 906 | 30.778 | 109.341 | 38.717 | 1.00 | 18.93 | 6 |
| ATOM | 10440 | C | PHE | C | 906 | 24.355 | 110.283 | 37.815 | 1.00 | 32.71 | 6 |
| ATOM | 10441 | O | PHE | C | 906 | 24.227 | 111.508 | 37.867 | 1.00 | 32.97 | 8 |
| ATOM | 10442 | N | ASP | C | 907 | 23.338 | 109.467 | 37.527 | 1.00 | 29.06 | 7 |
| ATOM | 10443 | CA | ASP | C | 907 | 22.032 | 110.044 | 37.230 | 1.00 | 30.90 | 6 |
| ATOM | 10444 | CB | ASP | C | 907 | 21.959 | 110.455 | 35.759 | 1.00 | 61.65 | 6 |
| ATOM | 10445 | CG | ASP | C | 907 | 20.898 | 111.511 | 35.497 | 1.00 | 62.92 | 6 |
| ATOM | 10446 | OD1 | ASP | C | 907 | 19.837 | 111.485 | 36.160 | 1.00 | 61.39 | 8 |
| ATOM | 10447 | OD2 | ASP | C | 907 | 21.131 | 112.365 | 34.614 | 1.00 | 63.98 | 8 |
| ATOM | 10448 | C | ASP | C | 907 | 20.816 | 109.192 | 37.537 | 1.00 | 32.34 | 6 |
| ATOM | 10449 | O | ASP | C | 907 | 20.277 | 108.536 | 36.646 | 1.00 | 32.53 | 8 |
| ATOM | 10450 | N | GLY | C | 908 | 20.373 | 109.224 | 38.793 | 1.00 | 45.67 | 7 |
| ATOM | 10451 | CA | GLY | C | 908 | 19.183 | 108.485 | 39.181 | 1.00 | 45.28 | 6 |
| ATOM | 10452 | C | GLY | C | 908 | 19.376 | 107.184 | 39.927 | 1.00 | 44.33 | 6 |
| ATOM | 10453 | O | GLY | C | 908 | 20.496 | 106.739 | 40.165 | 1.00 | 44.93 | 8 |
| ATOM | 10454 | N | ALA | C | 909 | 18.256 | 106.561 | 40.270 | 1.00 | 13.87 | 7 |
| ATOM | 10455 | CA | ALA | C | 909 | 18.273 | 105.320 | 41.004 | 1.00 | 13.87 | 6 |
| ATOM | 10456 | CB | ALA | C | 909 | 19.234 | 104.359 | 40.380 | 1.00 | 19.25 | 6 |
| ATOM | 10457 | C | ALA | C | 909 | 18.725 | 105.684 | 42.396 | 1.00 | 13.87 | 6 |
| ATOM | 10458 | O | ALA | C | 909 | 19.777 | 105.228 | 42.844 | 1.00 | 13.87 | 8 |
| ATOM | 10459 | N | THR | C | 910 | 17.930 | 106.516 | 43.072 | 1.00 | 22.51 | 7 |
| ATOM | 10460 | CA | THR | C | 910 | 18.258 | 106.966 | 44.419 | 1.00 | 25.35 | 6 |
| ATOM | 10461 | CB | THR | C | 910 | 17.766 | 108.374 | 44.686 | 1.00 | 54.99 | 6 |
| ATOM | 10462 | OG1 | THR | C | 910 | 17.878 | 109.145 | 43.488 | 1.00 | 58.09 | 8 |
| ATOM | 10463 | CG2 | THR | C | 910 | 18.621 | 109.021 | 45.769 | 1.00 | 57.49 | 6 |
| ATOM | 10464 | C | THR | C | 910 | 17.705 | 106.089 | 45.507 | 1.00 | 26.11 | 6 |
| ATOM | 10465 | O | THR | C | 910 | 16.551 | 105.674 | 45.485 | 1.00 | 26.89 | 8 |
| ATOM | 10466 | N | GLU | C | 911 | 18.553 | 105.856 | 46.490 | 1.00 | 42.54 | 7 |
| ATOM | 10467 | CA | GLU | C | 911 | 18.234 | 105.012 | 47.615 | 1.00 | 45.45 | 6 |
| ATOM | 10468 | CB | GLU | C | 911 | 19.106 | 105.406 | 48.794 | 1.00 | 56.31 | 6 |
| ATOM | 10469 | CG | GLU | C | 911 | 19.172 | 104.352 | 49.839 | 1.00 | 62.32 | 6 |
| ATOM | 10470 | CD | GLU | C | 911 | 20.208 | 104.664 | 50.857 | 1.00 | 65.43 | 6 |
| ATOM | 10471 | OE1 | GLU | C | 911 | 21.356 | 104.926 | 50.442 | 1.00 | 66.82 | 8 |
| ATOM | 10472 | OE2 | GLU | C | 911 | 19.874 | 104.646 | 52.062 | 1.00 | 68.96 | 8 |
| ATOM | 10473 | C | GLU | C | 911 | 16.768 | 104.981 | 48.025 | 1.00 | 45.59 | 6 |
| ATOM | 10474 | O | GLU | C | 911 | 16.223 | 103.909 | 48.275 | 1.00 | 47.49 | 8 |
| ATOM | 10475 | N | PRO | C | 912 | 16.109 | 106.148 | 48.099 | 1.00 | 16.94 | 7 |
| ATOM | 10476 | CD | PRO | C | 912 | 16.627 | 107.502 | 47.853 | 1.00 | 32.60 | 6 |
| ATOM | 10477 | CA | PRO | C | 912 | 14.699 | 106.219 | 48.484 | 1.00 | 17.33 | 6 |
| ATOM | 10478 | CB | PRO | C | 912 | 14.478 | 107.710 | 48.677 | 1.00 | 32.27 | 6 |
| ATOM | 10479 | CG | PRO | C | 912 | 15.356 | 108.298 | 47.643 | 1.00 | 32.01 | 6 |
| ATOM | 10480 | C | PRO | C | 912 | 13.778 | 105.631 | 47.423 | 1.00 | 18.58 | 6 |
| ATOM | 10481 | O | PRO | C | 912 | 12.792 | 104.965 | 47.755 | 1.00 | 18.06 | 8 |
| ATOM | 10482 | N | GLU | C | 913 | 14.099 | 105.895 | 46.156 | 1.00 | 40.96 | 7 |
| ATOM | 10483 | CA | GLU | C | 913 | 13.328 | 105.373 | 45.035 | 1.00 | 42.81 | 6 |
| ATOM | 10484 | CB | GLU | C | 913 | 13.897 | 105.898 | 43.725 | 1.00 | 69.38 | 6 |
| ATOM | 10485 | CG | GLU | C | 913 | 13.359 | 107.257 | 43.353 | 1.00 | 74.36 | 6 |
| ATOM | 10486 | CD | GLU | C | 913 | 14.261 | 108.011 | 42.391 | 1.00 | 77.79 | 6 |
| ATOM | 10487 | OE1 | GLU | C | 913 | 14.766 | 107.397 | 41.420 | 1.00 | 79.14 | 8 |
| ATOM | 10488 | OE2 | GLU | C | 913 | 14.450 | 109.232 | 42.603 | 1.00 | 79.35 | 8 |
| ATOM | 10489 | C | GLU | C | 913 | 13.431 | 103.854 | 45.083 | 1.00 | 42.98 | 6 |
| ATOM | 10490 | O | GLU | C | 913 | 12.441 | 103.133 | 44.903 | 1.00 | 43.19 | 8 |
| ATOM | 10491 | N | ILE | C | 914 | 14.643 | 103.371 | 45.335 | 1.00 | 37.29 | 7 |
| ATOM | 10492 | CA | ILE | C | 914 | 14.879 | 101.940 | 45.443 | 1.00 | 35.51 | 6 |
| ATOM | 10493 | CB | ILE | C | 914 | 16.361 | 101.663 | 45.775 | 1.00 | 14.77 | 6 |
| ATOM | 10494 | CG2 | ILE | C | 914 | 16.643 | 100.161 | 45.721 | 1.00 | 13.87 | 6 |
| ATOM | 10495 | CG1 | ILE | C | 914 | 17.248 | 102.406 | 44.776 | 1.00 | 14.08 | 6 |
| ATOM | 10496 | CD1 | ILE | C | 914 | 18.680 | 102.473 | 45.189 | 1.00 | 15.40 | 6 |
| ATOM | 10497 | C | ILE | C | 914 | 13.967 | 101.447 | 46.570 | 1.00 | 36.23 | 6 |
| ATOM | 10498 | O | ILE | C | 914 | 13.289 | 100.437 | 46.440 | 1.00 | 35.85 | 8 |
| ATOM | 10499 | N | LYS | C | 915 | 13.947 | 102.182 | 47.673 | 1.00 | 21.63 | 7 |
| ATOM | 10500 | CA | LYS | C | 915 | 13.097 | 101.833 | 48.788 | 1.00 | 23.86 | 6 |
| ATOM | 10501 | CB | LYS | C | 915 | 13.162 | 102.919 | 49.859 | 1.00 | 45.95 | 6 |
| ATOM | 10502 | CG | LYS | C | 915 | 14.537 | 103.087 | 50.501 | 1.00 | 45.43 | 6 |
| ATOM | 10503 | CD | LYS | C | 915 | 14.996 | 101.819 | 51.235 | 1.00 | 43.48 | 6 |
| ATOM | 10504 | CE | LYS | C | 915 | 16.163 | 102.067 | 52.227 | 1.00 | 42.64 | 6 |
| ATOM | 10505 | NZ | LYS | C | 915 | 17.487 | 102.423 | 51.632 | 1.00 | 40.26 | 7 |
| ATOM | 10506 | C | LYS | C | 915 | 11.658 | 101.680 | 48.277 | 1.00 | 26.64 | 6 |
| ATOM | 10507 | O | LYS | C | 915 | 11.008 | 100.654 | 48.523 | 1.00 | 26.46 | 8 |
| ATOM | 10508 | N | GLU | C | 916 | 11.149 | 102.689 | 47.568 | 1.00 | 71.98 | 7 |
| ATOM | 10509 | CA | GLU | C | 916 | 9.787 | 102.597 | 47.038 | 1.00 | 73.89 | 6 |
| ATOM | 10510 | CB | GLU | C | 916 | 9.456 | 103.776 | 46.114 | 1.00 | 95.83 | 6 |
| ATOM | 10511 | CG | GLU | C | 916 | 8.055 | 103.667 | 45.494 | 1.00 | 99.06 | 6 |
| ATOM | 10512 | CD | GLU | C | 916 | 7.796 | 104.649 | 44.351 | 1.00 | 101.48 | 6 |
| ATOM | 10513 | OE1 | GLU | C | 916 | 8.606 | 104.700 | 43.395 | 1.00 | 102.44 | 8 |
| ATOM | 10514 | OE2 | GLU | C | 916 | 6.765 | 105.359 | 44.399 | 1.00 | 103.28 | 8 |
| ATOM | 10515 | C | GLU | C | 916 | 9.697 | 101.301 | 46.243 | 1.00 | 74.32 | 6 |
| ATOM | 10516 | O | GLU | C | 916 | 8.837 | 100.458 | 46.500 | 1.00 | 74.55 | 8 |
| ATOM | 10517 | N | LEU | C | 917 | 10.599 | 101.151 | 45.277 | 1.00 | 76.75 | 7 |
| ATOM | 10518 | CA | LEU | C | 917 | 10.654 | 99.960 | 44.444 | 1.00 | 76.02 | 6 |
| ATOM | 10519 | CB | LEU | C | 917 | 11.857 | 100.059 | 43.497 | 1.00 | 31.71 | 6 |
| ATOM | 10520 | CG | LEU | C | 917 | 11.583 | 100.671 | 42.120 | 1.00 | 30.77 | 6 |
| ATOM | 10521 | CD1 | LEU | C | 917 | 12.875 | 101.020 | 41.411 | 1.00 | 30.00 | 6 |
| ATOM | 10522 | CD2 | LEU | C | 917 | 10.772 | 99.672 | 41.302 | 1.00 | 30.45 | 6 |
| ATOM | 10523 | C | LEU | C | 917 | 10.743 | 98.700 | 45.328 | 1.00 | 75.62 | 6 |
| ATOM | 10524 | O | LEU | C | 917 | 10.494 | 97.588 | 44.880 | 1.00 | 75.68 | 8 |
| ATOM | 10525 | N | LEU | C | 918 | 11.100 | 98.919 | 46.590 | 1.00 | 28.41 | 7 |
| ATOM | 10526 | CA | LEU | C | 918 | 11.201 | 97.849 | 47.570 | 1.00 | 28.59 | 6 |
| ATOM | 10527 | CB | LEU | C | 918 | 12.400 | 98.081 | 48.483 | 1.00 | 37.23 | 6 |
| ATOM | 10528 | CG | LEU | C | 918 | 13.752 | 97.539 | 48.051 | 1.00 | 35.84 | 6 |
| ATOM | 10529 | CD1 | LEU | C | 918 | 14.831 | 98.022 | 49.006 | 1.00 | 36.16 | 6 |
| ATOM | 10530 | CD2 | LEU | C | 918 | 13.676 | 96.032 | 48.045 | 1.00 | 36.60 | 6 |
| ATOM | 10531 | C | LEU | C | 918 | 9.918 | 97.819 | 48.404 | 1.00 | 28.80 | 6 |
| ATOM | 10532 | O | LEU | C | 918 | 9.526 | 96.772 | 48.940 | 1.00 | 27.28 | 8 |
| ATOM | 10533 | N | ALA | C | 919 | 9.279 | 98.976 | 48.541 | 1.00 | 53.35 | 7 |
| ATOM | 10534 | CA | ALA | C | 919 | 8.024 | 99.028 | 49.263 | 1.00 | 54.87 | 6 |
| ATOM | 10535 | CB | ALA | C | 919 | 7.435 | 100.411 | 49.194 | 1.00 | 26.84 | 6 |
| ATOM | 10536 | C | ALA | C | 919 | 7.197 | 98.042 | 48.446 | 1.00 | 56.11 | 6 |
| ATOM | 10537 | O | ALA | C | 919 | 6.677 | 97.066 | 48.987 | 1.00 | 56.33 | 8 |
| ATOM | 10538 | N | GLU | C | 920 | 7.108 | 98.291 | 47.135 | 1.00 | 51.97 | 7 |
| ATOM | 10539 | CA | GLU | C | 920 | 6.401 | 97.383 | 46.229 | 1.00 | 52.59 | 6 |
| ATOM | 10540 | CB | GLU | C | 920 | 6.403 | 97.892 | 44.780 | 1.00 | 76.15 | 6 |
| ATOM | 10541 | CG | GLU | C | 920 | 5.676 | 99.214 | 44.537 | 1.00 | 78.37 | 6 |
| ATOM | 10542 | CD | GLU | C | 920 | 5.596 | 99.579 | 43.054 | 1.00 | 80.29 | 6 |
| ATOM | 10543 | OE1 | GLU | C | 920 | 4.929 | 98.841 | 42.299 | 1.00 | 79.99 | 8 |
| ATOM | 10544 | OE2 | GLU | C | 920 | 6.198 | 100.596 | 42.640 | 1.00 | 80.59 | 8 |
| ATOM | 10545 | C | GLU | C | 920 | 7.252 | 96.125 | 46.320 | 1.00 | 51.92 | 6 |
| ATOM | 10546 | O | GLU | C | 920 | 8.288 | 96.138 | 46.979 | 1.00 | 51.86 | 8 |
| ATOM | 10547 | N | ALA | C | 921 | 6.850 | 95.050 | 45.654 | 1.00 | 69.88 | 7 |
| ATOM | 10548 | CA | ALA | C | 921 | 7.603 | 93.802 | 45.750 | 1.00 | 68.97 | 6 |
| ATOM | 10549 | CB | ALA | C | 921 | 8.953 | 93.935 | 45.064 | 1.00 | 58.25 | 6 |
| ATOM | 10550 | C | ALA | C | 921 | 7.776 | 93.554 | 47.248 | 1.00 | 68.54 | 6 |
| ATOM | 10551 | O | ALA | C | 921 | 8.758 | 93.961 | 47.864 | 1.00 | 69.84 | 8 |
| ATOM | 10552 | N | PHE | C | 922 | 6.770 | 92.914 | 47.821 | 1.00 | 60.62 | 7 |
| ATOM | 10553 | CA | PHE | C | 922 | 6.698 | 92.583 | 49.238 | 1.00 | 59.45 | 6 |
| ATOM | 10554 | CB | PHE | C | 922 | 7.273 | 93.670 | 50.152 | 1.00 | 33.67 | 6 |
| ATOM | 10555 | CG | PHE | C | 922 | 7.014 | 93.408 | 51.625 | 1.00 | 31.30 | 6 |
| ATOM | 10556 | CD1 | PHE | C | 922 | 7.656 | 92.364 | 52.285 | 1.00 | 30.29 | 6 |
| ATOM | 10557 | CD2 | PHE | C | 922 | 6.074 | 94.149 | 52.326 | 1.00 | 30.37 | 6 |
| ATOM | 10558 | CE1 | PHE | C | 922 | 7.356 | 92.062 | 53.613 | 1.00 | 29.50 | 6 |
| ATOM | 10559 | CE2 | PHE | C | 922 | 5.775 | 93.847 | 53.651 | 1.00 | 29.79 | 6 |
| ATOM | 10560 | CZ | PHE | C | 922 | 6.414 | 92.805 | 54.294 | 1.00 | 29.06 | 6 |
| ATOM | 10561 | C | PHE | C | 922 | 5.212 | 92.526 | 49.462 | 1.00 | 60.82 | 6 |
| ATOM | 10562 | O | PHE | C | 922 | 4.690 | 91.560 | 50.013 | 1.00 | 61.90 | 8 |
| ATOM | 10563 | N | ASN | C | 923 | 4.527 | 93.579 | 49.029 | 1.00 | 97.16 | 7 |
| ATOM | 10564 | CA | ASN | C | 923 | 3.083 | 93.620 | 49.151 | 1.00 | 97.30 | 6 |
| ATOM | 10565 | CB | ASN | C | 923 | 2.534 | 94.965 | 48.671 | 1.00 | 91.91 | 6 |
| ATOM | 10566 | CG | ASN | C | 923 | 2.884 | 96.109 | 49.618 | 1.00 | 92.86 | 6 |
| ATOM | 10567 | OD1 | ASN | C | 923 | 2.640 | 96.034 | 50.831 | 1.00 | 93.08 | 8 |
| ATOM | 10568 | ND2 | ASN | C | 923 | 3.453 | 97.175 | 49.069 | 1.00 | 91.46 | 7 |
| ATOM | 10569 | C | ASN | C | 923 | 2.631 | 92.488 | 48.251 | 1.00 | 97.23 | 6 |
| ATOM | 10570 | O | ASN | C | 923 | 1.440 | 92.234 | 48.081 | 1.00 | 98.02 | 8 |
| ATOM | 10571 | N | LEU | C | 924 | 3.630 | 91.817 | 47.683 | 1.00 | 47.77 | 7 |
| ATOM | 10572 | CA | LEU | C | 924 | 3.447 | 90.677 | 46.809 | 1.00 | 46.89 | 6 |
| ATOM | 10573 | CB | LEU | C | 924 | 3.761 | 91.044 | 45.361 | 1.00 | 40.81 | 6 |
| ATOM | 10574 | CG | LEU | C | 924 | 4.750 | 92.156 | 45.053 | 1.00 | 39.42 | 6 |
| ATOM | 10575 | CD1 | LEU | C | 924 | 4.741 | 92.348 | 43.557 | 1.00 | 40.38 | 6 |
| ATOM | 10576 | CD2 | LEU | C | 924 | 4.358 | 93.454 | 45.718 | 1.00 | 38.41 | 6 |
| ATOM | 10577 | C | LEU | C | 924 | 4.357 | 89.567 | 47.293 | 1.00 | 47.08 | 6 |
| ATOM | 10578 | O | LEU | C | 924 | 3.906 | 88.442 | 47.496 | 1.00 | 46.92 | 8 |
| ATOM | 10579 | N | TYR | C | 925 | 5.637 | 89.877 | 47.489 | 1.00 | 48.13 | 7 |
| ATOM | 10580 | CA | TYR | C | 925 | 6.569 | 88.867 | 47.994 | 1.00 | 48.95 | 6 |
| ATOM | 10581 | CB | TYR | C | 925 | 7.946 | 89.476 | 48.305 | 1.00 | 49.10 | 6 |
| ATOM | 10582 | CG | TYR | C | 925 | 8.714 | 88.723 | 49.374 | 1.00 | 49.73 | 6 |
| ATOM | 10583 | CD1 | TYR | C | 925 | 8.926 | 87.343 | 49.275 | 1.00 | 50.22 | 6 |
| ATOM | 10584 | CE1 | TYR | C | 925 | 9.569 | 86.624 | 50.300 | 1.00 | 50.15 | 6 |
| ATOM | 10585 | CD2 | TYR | C | 925 | 9.171 | 89.372 | 50.517 | 1.00 | 50.11 | 6 |
| ATOM | 10586 | CE2 | TYR | C | 925 | 9.813 | 88.665 | 51.545 | 1.00 | 50.38 | 6 |
| ATOM | 10587 | CZ | TYR | C | 925 | 10.004 | 87.291 | 51.432 | 1.00 | 50.35 | 6 |
| ATOM | 10588 | OH | TYR | C | 925 | 10.588 | 86.589 | 52.462 | 1.00 | 49.43 | 8 |
| ATOM | 10589 | C | TYR | C | 925 | 5.945 | 88.327 | 49.269 | 1.00 | 49.87 | 6 |
| ATOM | 10590 | O | TYR | C | 925 | 5.804 | 87.121 | 49.442 | 1.00 | 49.62 | 8 |
| ATOM | 10591 | N | PHE | C | 926 | 5.571 | 89.243 | 50.154 | 1.00 | 70.22 | 7 |
| ATOM | 10592 | CA | PHE | C | 926 | 4.935 | 88.883 | 51.407 | 1.00 | 73.05 | 6 |
| ATOM | 10593 | CB | PHE | C | 926 | 5.209 | 89.934 | 52.482 | 1.00 | 49.65 | 6 |
| ATOM | 10594 | CG | PHE | C | 926 | 4.766 | 89.513 | 53.844 | 1.00 | 49.63 | 6 |
| ATOM | 10595 | CD1 | PHE | C | 926 | 5.479 | 88.542 | 54.555 | 1.00 | 49.72 | 6 |
| ATOM | 10596 | CD2 | PHE | C | 926 | 3.604 | 90.031 | 54.400 | 1.00 | 50.08 | 6 |
| ATOM | 10597 | CE1 | PHE | C | 926 | 5.035 | 88.090 | 55.806 | 1.00 | 50.09 | 6 |
| ATOM | 10598 | CE2 | PHE | C | 926 | 3.149 | 89.587 | 55.649 | 1.00 | 50.98 | 6 |
| ATOM | 10599 | CZ | PHE | C | 926 | 3.867 | 88.613 | 56.354 | 1.00 | 50.94 | 6 |
| ATOM | 10600 | C | PHE | C | 926 | 3.435 | 88.805 | 51.157 | 1.00 | 75.68 | 6 |
| ATOM | 10601 | O | PHE | C | 926 | 2.691 | 88.224 | 51.950 | 1.00 | 76.44 | 8 |
| ATOM | 10602 | N | GLY | C | 927 | 2.997 | 89.400 | 50.050 | 1.00 | 51.62 | 7 |
| ATOM | 10603 | CA | GLY | C | 927 | 1.585 | 89.386 | 49.712 | 1.00 | 53.05 | 6 |
| ATOM | 10604 | C | GLY | C | 927 | 1.163 | 87.959 | 49.467 | 1.00 | 54.38 | 6 |
| ATOM | 10605 | O | GLY | C | 927 | 0.029 | 87.571 | 49.738 | 1.00 | 53.67 | 8 |
| ATOM | 10606 | N | LYS | C | 928 | 2.105 | 87.174 | 48.963 | 1.00 | 59.07 | 7 |
| ATOM | 10607 | CA | LYS | C | 928 | 1.858 | 85.777 | 48.666 | 1.00 | 62.02 | 6 |
| ATOM | 10608 | CB | LYS | C | 928 | 3.032 | 85.207 | 47.865 | 1.00 | 171.68 | 6 |
| ATOM | 10609 | CG | LYS | C | 928 | 3.184 | 85.856 | 46.498 | 1.00 | 173.79 | 6 |
| ATOM | 10610 | CD | LYS | C | 928 | 4.420 | 85.380 | 45.759 | 1.00 | 175.33 | 6 |
| ATOM | 10611 | CE | LYS | C | 928 | 4.557 | 86.110 | 44.428 | 1.00 | 175.69 | 6 |
| ATOM | 10612 | NZ | LYS | C | 928 | 5.766 | 85.701 | 43.661 | 1.00 | 176.89 | 7 |
| ATOM | 10613 | C | LYS | C | 928 | 1.594 | 84.932 | 49.915 | 1.00 | 62.87 | 6 |
| ATOM | 10614 | O | LYS | C | 928 | 0.695 | 84.093 | 49.905 | 1.00 | 62.54 | 8 |
| ATOM | 10615 | N | ARG | C | 929 | 2.361 | 85.139 | 50.983 | 1.00 | 75.41 | 7 |
| ATOM | 10616 | CA | ARG | C | 929 | 2.149 | 84.365 | 52.204 | 1.00 | 77.55 | 6 |
| ATOM | 10617 | CB | ARG | C | 929 | 2.950 | 84.936 | 53.367 | 1.00 | 98.13 | 6 |
| ATOM | 10618 | CG | ARC | C | 929 | 4.445 | 84.810 | 53.252 | 1.00 | 100.19 | 6 |
| ATOM | 10619 | CD | ARG | C | 929 | 5.047 | 85.113 | 54.600 | 1.00 | 101.93 | 6 |
| ATOM | 10620 | NE | ARG | C | 929 | 6.473 | 85.380 | 54.531 | 1.00 | 104.27 | 7 |
| ATOM | 10621 | CZ | ARG | C | 929 | 7.211 | 85.688 | 55.589 | 1.00 | 105.59 | 6 |
| ATOM | 10622 | NH1 | ARG | C | 929 | 6.645 | 85.762 | 56.786 | 1.00 | 106.10 | 7 |
| ATOM | 10623 | NH2 | ARG | C | 929 | 8.508 | 85.927 | 55.451 | 1.00 | 106.46 | 7 |
| ATOM | 10624 | C | ARG | C | 929 | 0.679 | 84.422 | 52.577 | 1.00 | 78.07 | 6 |
| ATOM | 10625 | O | ARG | C | 929 | 0.012 | 83.402 | 52.746 | 1.00 | 77.62 | 8 |
| ATOM | 10626 | N | GLN | C | 930 | 0.182 | 85.639 | 52.713 | 1.00 | 121.34 | 7 |
| ATOM | 10627 | CA | GLN | C | 930 | −1.207 | 85.851 | 53.055 | 1.00 | 122.36 | 6 |
| ATOM | 10628 | CB | GLN | C | 930 | −1.381 | 87.300 | 53.500 | 1.00 | 77.50 | 6 |
| ATOM | 10629 | CG | GLN | C | 930 | −0.426 | 87.669 | 54.638 | 1.00 | 78.01 | 6 |
| ATOM | 10630 | CD | GLN | C | 930 | −0.246 | 89.163 | 54.808 | 1.00 | 77.89 | 6 |
| ATOM | 10631 | OE1 | GLN | C | 930 | 0.218 | 89.850 | 53.899 | 1.00 | 78.54 | 8 |
| ATOM | 10632 | NE2 | GLN | C | 930 | −0.608 | 89.674 | 55.979 | 1.00 | 77.85 | 7 |
| ATOM | 10633 | C | GLN | C | 930 | −2.028 | 85.528 | 51.808 | 1.00 | 122.93 | 6 |
| ATOM | 10634 | O | GLN | C | 930 | −3.102 | 86.080 | 51.578 | 1.00 | 124.12 | 8 |
| ATOM | 10635 | N | GLY | C | 931 | −1.483 | 84.621 | 51.005 | 1.00 | 137.15 | 7 |
| ATOM | 10636 | CA | GLY | C | 931 | −2.126 | 84.178 | 49.784 | 1.00 | 136.42 | 6 |
| ATOM | 10637 | C | GLY | C | 931 | −2.051 | 82.665 | 49.784 | 1.00 | 136.00 | 6 |
| ATOM | 10638 | O | GLY | C | 931 | −3.078 | 81.992 | 49.850 | 1.00 | 136.17 | 8 |
| ATOM | 10639 | N | GLU | C | 932 | −0.835 | 82.126 | 49.717 | 1.00 | 96.18 | 7 |
| ATOM | 10640 | CA | GLU | C | 932 | −0.652 | 80.679 | 49.748 | 1.00 | 94.52 | 6 |
| ATOM | 10641 | CB | GLU | C | 932 | 0.832 | 80.308 | 49.755 | 1.00 | 76.46 | 6 |
| ATOM | 10642 | CG | GLU | C | 932 | 1.612 | 80.799 | 48.551 | 1.00 | 76.62 | 6 |
| ATOM | 10643 | CD | GLU | C | 932 | 3.082 | 80.389 | 48.588 | 1.00 | 76.70 | 6 |
| ATOM | 10644 | OE1 | GLU | C | 932 | 3.776 | 80.708 | 49.581 | 1.00 | 76.25 | 8 |
| ATOM | 10645 | OE2 | GLU | C | 932 | 3.543 | 79.752 | 47.618 | 1.00 | 76.53 | 8 |
| ATOM | 10646 | C | GLU | C | 932 | −1.292 | 80.203 | 51.043 | 1.00 | 93.61 | 6 |
| ATOM | 10647 | O | GLU | C | 932 | −1.656 | 79.035 | 51.177 | 1.00 | 93.06 | 8 |
| ATOM | 10648 | N | GLY | C | 933 | −1.421 | 81.131 | 51.990 | 1.00 | 182.25 | 7 |
| ATOM | 10649 | CA | GLY | C | 933 | −2.022 | 80.828 | 53.275 | 1.00 | 180.72 | 6 |
| ATOM | 10650 | C | GLY | C | 933 | −1.011 | 80.659 | 54.392 | 1.00 | 179.07 | 6 |
| ATOM | 10651 | O | GLY | C | 933 | −1.071 | 79.679 | 55.136 | 1.00 | 179.86 | 8 |
| ATOM | 10652 | N | PHE | C | 934 | −0.082 | 81.605 | 54.518 | 1.00 | 81.73 | 7 |
| ATOM | 10653 | CA | PHE | C | 934 | 0.939 | 81.529 | 55.563 | 1.00 | 78.85 | 6 |
| ATOM | 10654 | CB | PHE | C | 934 | 2.186 | 80.805 | 55.044 | 1.00 | 70.78 | 6 |
| ATOM | 10655 | CG | PHE | C | 934 | 1.906 | 79.438 | 54.477 | 1.00 | 69.68 | 6 |
| ATOM | 10656 | CD1 | PHE | C | 934 | 1.658 | 79.268 | 53.119 | 1.00 | 69.14 | 6 |
| ATOM | 10657 | CD2 | PHE | C | 934 | 1.875 | 78.319 | 55.306 | 1.00 | 69.73 | 6 |
| ATOM | 10658 | CE1 | PHE | C | 934 | 1.385 | 78.006 | 52.596 | 1.00 | 68.85 | 6 |
| ATOM | 10659 | CE2 | PHE | C | 934 | 1.603 | 77.052 | 54.790 | 1.00 | 69.66 | 6 |
| ATOM | 10660 | CZ | PHE | C | 934 | 1.358 | 76.898 | 53.432 | 1.00 | 68.78 | 6 |
| ATOM | 10661 | C | PHE | C | 934 | 1.344 | 82.894 | 56.115 | 1.00 | 77.20 | 6 |
| ATOM | 10662 | O | PHE | C | 934 | 0.860 | 83.933 | 55.658 | 1.00 | 77.60 | 8 |
| ATOM | 10663 | N | GLY | C | 935 | 2.239 | 82.877 | 57.102 | 1.00 | 94.36 | 7 |
| ATOM | 10664 | CA | GLY | C | 935 | 2.701 | 84.116 | 57.706 | 1.00 | 91.73 | 6 |
| ATOM | 10665 | C | GLY | C | 935 | 3.684 | 83.935 | 58.851 | 1.00 | 89.40 | 6 |
| ATOM | 10666 | O | GLY | C | 935 | 3.509 | 83.044 | 59.683 | 1.00 | 89.89 | 8 |
| ATOM | 10667 | N | VAL | C | 936 | 4.709 | 84.789 | 58.883 | 1.00 | 34.92 | 7 |
| ATOM | 10668 | CA | VAL | C | 936 | 5.756 | 84.769 | 59.904 | 1.00 | 31.78 | 6 |
| ATOM | 10669 | CB | VAL | C | 936 | 5.643 | 85.989 | 60.820 | 1.00 | 33.96 | 6 |
| ATOM | 10670 | CG1 | VAL | C | 936 | 6.439 | 85.766 | 62.093 | 1.00 | 34.87 | 6 |
| ATOM | 10671 | CG2 | VAL | C | 936 | 6.152 | 87.218 | 60.098 | 1.00 | 34.76 | 6 |
| ATOM | 10672 | C | VAL | C | 936 | 5.805 | 83.533 | 60.789 | 1.00 | 30.62 | 6 |
| ATOM | 10673 | O | VAL | C | 936 | 4.878 | 83.279 | 61.560 | 1.00 | 29.99 | 8 |
| ATOM | 10674 | N | ASP | C | 937 | 6.889 | 82.765 | 60.700 | 1.00 | 37.82 | 7 |
| ATOM | 10675 | CA | ASP | C | 937 | 6.982 | 81.570 | 61.532 | 1.00 | 37.19 | 6 |
| ATOM | 10676 | CB | ASP | C | 937 | 7.300 | 80.298 | 60.698 | 1.00 | 68.96 | 6 |
| ATOM | 10677 | CG | ASP | C | 937 | 8.745 | 80.205 | 60.246 | 1.00 | 69.34 | 6 |
| ATOM | 10678 | OD1 | ASP | C | 937 | 9.644 | 80.352 | 61.093 | 1.00 | 70.00 | 8 |
| ATOM | 10679 | OD2 | ASP | C | 937 | 8.977 | 79.952 | 59.045 | 1.00 | 69.42 | 8 |
| ATOM | 10680 | C | ASP | C | 937 | 7.950 | 81.726 | 62.683 | 1.00 | 36.14 | 6 |
| ATOM | 10681 | O | ASP | C | 937 | 8.763 | 82.647 | 62.704 | 1.00 | 34.12 | 8 |
| ATOM | 10682 | N | LYS | C | 938 | 7.831 | 80.817 | 63.643 | 1.00 | 49.63 | 7 |
| ATOM | 10683 | CA | LYS | C | 938 | 8.638 | 80.826 | 64.848 | 1.00 | 51.96 | 6 |
| ATOM | 10684 | CB | LYS | C | 938 | 8.492 | 79.480 | 65.567 | 1.00 | 78.51 | 6 |
| ATOM | 10685 | CG | LYS | C | 938 | 7.035 | 79.154 | 65.921 | 1.00 | 79.62 | 6 |
| ATOM | 10686 | CD | LYS | C | 938 | 6.884 | 77.942 | 66.850 | 1.00 | 80.06 | 6 |
| ATOM | 10687 | CE | LYS | C | 938 | 7.147 | 78.286 | 68.328 | 1.00 | 80.47 | 6 |
| ATOM | 10688 | NZ | LYS | C | 938 | 6.106 | 79.166 | 68.954 | 1.00 | 81.04 | 7 |
| ATOM | 10689 | C | LYS | C | 938 | 10.112 | 81.196 | 64.660 | 1.00 | 53.10 | 6 |
| ATOM | 10690 | O | LYS | C | 938 | 10.799 | 81.510 | 65.637 | 1.00 | 53.64 | 8 |
| ATOM | 10691 | N | ARG | C | 939 | 10.595 | 81.186 | 63.419 | 1.00 | 54.14 | 7 |
| ATOM | 10692 | CA | ARG | C | 939 | 11.984 | 81.546 | 63.135 | 1.00 | 54.30 | 6 |
| ATOM | 10693 | CB | ARG | C | 939 | 12.458 | 80.847 | 61.873 | 1.00 | 51.97 | 6 |
| ATOM | 10694 | CG | ARG | C | 939 | 12.390 | 79.357 | 61.949 | 1.00 | 53.03 | 6 |
| ATOM | 10695 | CD | ARG | C | 939 | 11.810 | 78.850 | 60.674 | 1.00 | 54.13 | 6 |
| ATOM | 10696 | NE | ARG | C | 939 | 12.529 | 77.699 | 60.181 | 1.00 | 54.99 | 7 |
| ATOM | 10697 | CZ | ARG | C | 939 | 12.297 | 77.165 | 58.996 | 1.00 | 57.20 | 6 |
| ATOM | 10698 | NH1 | ARG | C | 939 | 11.370 | 77.695 | 58.213 | 1.00 | 57.62 | 7 |
| ATOM | 10699 | NH2 | ARG | C | 939 | 12.983 | 76.110 | 58.596 | 1.00 | 59.96 | 7 |
| ATOM | 10700 | C | ARG | C | 939 | 12.118 | 83.054 | 62.941 | 1.00 | 54.06 | 6 |
| ATOM | 10701 | O | ARG | C | 939 | 12.950 | 83.706 | 63.576 | 1.00 | 55.43 | 8 |
| ATOM | 10702 | N | GLU | C | 940 | 11.305 | 83.596 | 62.042 | 1.00 | 20.90 | 7 |
| ATOM | 10703 | CA | GLU | C | 940 | 11.314 | 85.022 | 61.775 | 1.00 | 20.56 | 6 |
| ATOM | 10704 | CB | GLU | C | 940 | 10.301 | 85.363 | 60.676 | 1.00 | 46.34 | 6 |
| ATOM | 10705 | CG | GLU | C | 940 | 10.352 | 84.402 | 59.500 | 1.00 | 44.16 | 6 |
| ATOM | 10706 | CD | GLU | C | 940 | 9.423 | 84.787 | 58.372 | 1.00 | 41.54 | 6 |
| ATOM | 10707 | OE1 | GLU | C | 940 | 9.785 | 85.691 | 57.599 | 1.00 | 41.51 | 8 |
| ATOM | 10708 | OE2 | GLU | C | 940 | 8.335 | 84.186 | 58.258 | 1.00 | 40.41 | 8 |
| ATOM | 10709 | C | GLU | C | 940 | 10.950 | 85.745 | 63.076 | 1.00 | 21.44 | 6 |
| ATOM | 10710 | O | GLU | C | 940 | 11.182 | 86.942 | 63.226 | 1.00 | 20.60 | 8 |
| ATOM | 10711 | N | LYS | C | 941 | 10.357 | 85.027 | 64.021 | 1.00 | 85.59 | 7 |
| ATOM | 10712 | CA | LYS | C | 941 | 10.042 | 85.669 | 65.282 | 1.00 | 87.93 | 6 |
| ATOM | 10713 | CB | LYS | C | 941 | 9.091 | 84.824 | 66.129 | 1.00 | 88.47 | 6 |
| ATOM | 10714 | CG | LYS | C | 941 | 7.633 | 85.103 | 65.821 | 1.00 | 89.73 | 6 |
| ATOM | 10715 | CD | LYS | C | 941 | 6.770 | 85.026 | 67.065 | 1.00 | 91.30 | 6 |
| ATOM | 10716 | CE | LYS | C | 941 | 5.392 | 85.615 | 66.795 | 1.00 | 92.39 | 6 |
| ATOM | 10717 | NZ | LYS | C | 941 | 4.610 | 85.825 | 68.048 | 1.00 | 94.10 | 7 |
| ATOM | 10718 | C | LYS | C | 941 | 11.368 | 85.836 | 65.988 | 1.00 | 88.95 | 6 |
| ATOM | 10719 | O | LYS | C | 941 | 11.709 | 86.940 | 66.410 | 1.00 | 90.48 | 8 |
| ATOM | 10720 | N | GLU | C | 942 | 12.123 | 84.743 | 66.097 | 1.00 | 54.45 | 7 |
| ATOM | 10721 | CA | GLU | C | 942 | 13.425 | 84.797 | 66.742 | 1.00 | 53.66 | 6 |
| ATOM | 10722 | CB | GLU | C | 942 | 14.197 | 83.497 | 66.536 | 1.00 | 113.49 | 6 |
| ATOM | 10723 | CG | GLU | C | 942 | 13.642 | 82.318 | 67.296 | 1.00 | 117.32 | 6 |
| ATOM | 10724 | CD | GLU | C | 942 | 14.718 | 81.323 | 67.680 | 1.00 | 119.34 | 6 |
| ATOM | 10725 | OE1 | GLU | C | 942 | 15.474 | 80.878 | 66.790 | 1.00 | 120.23 | 8 |
| ATOM | 10726 | OE2 | GLU | C | 942 | 14.808 | 80.987 | 68.878 | 1.00 | 120.12 | 8 |
| ATOM | 10727 | C | GLU | C | 942 | 14.229 | 85.942 | 66.154 | 1.00 | 52.80 | 6 |
| ATOM | 10728 | O | GLU | C | 942 | 14.663 | 86.846 | 66.871 | 1.00 | 53.14 | 8 |
| ATOM | 10729 | N | VAL | C | 943 | 14.416 | 85.903 | 64.839 | 1.00 | 65.20 | 7 |
| ATOM | 10730 | CA | VAL | C | 943 | 15.179 | 86.935 | 64.162 | 1.00 | 63.66 | 6 |
| ATOM | 10731 | CB | VAL | C | 943 | 15.104 | 86.765 | 62.640 | 1.00 | 61.57 | 6 |
| ATOM | 10732 | CG1 | VAL | C | 943 | 15.758 | 87.945 | 61.941 | 1.00 | 61.54 | 6 |
| ATOM | 10733 | CG2 | VAL | C | 943 | 15.810 | 85.478 | 62.252 | 1.00 | 61.38 | 6 |
| ATOM | 10734 | C | VAL | C | 943 | 14.724 | 88.328 | 64.567 | 1.00 | 62.96 | 6 |
| ATOM | 10735 | O | VAL | C | 943 | 15.293 | 88.893 | 65.480 | 1.00 | 63.98 | 8 |
| ATOM | 10736 | N | LEU | C | 944 | 13.708 | 88.873 | 63.906 | 1.00 | 13.88 | 7 |
| ATOM | 10737 | CA | LEU | C | 944 | 13.207 | 90.211 | 64.224 | 1.00 | 13.87 | 6 |
| ATOM | 10738 | CB | LEU | C | 944 | 11.698 | 90.287 | 64.018 | 1.00 | 46.25 | 6 |
| ATOM | 10739 | CG | LEU | C | 944 | 11.102 | 90.011 | 62.642 | 1.00 | 47.00 | 6 |
| ATOM | 10740 | CD1 | LEU | C | 944 | 9.596 | 89.914 | 62.772 | 1.00 | 48.47 | 6 |
| ATOM | 10741 | CD2 | LEU | C | 944 | 11.493 | 91.105 | 61.674 | 1.00 | 47.73 | 6 |
| ATOM | 10742 | C | LEU | C | 944 | 13.501 | 90.618 | 65.657 | 1.00 | 13.87 | 6 |
| ATOM | 10743 | O | LEU | C | 944 | 14.149 | 91.625 | 65.909 | 1.00 | 13.87 | 8 |
| ATOM | 10744 | N | ALA | C | 945 | 13.018 | 89.825 | 66.600 | 1.00 | 31.01 | 7 |
| ATOM | 10745 | CA | ALA | C | 945 | 13.224 | 90.105 | 68.012 | 1.00 | 31.66 | 6 |
| ATOM | 10746 | CB | ALA | C | 945 | 12.505 | 89.050 | 68.857 | 1.00 | 40.26 | 6 |
| ATOM | 10747 | C | ALA | C | 945 | 14.703 | 90.139 | 68.371 | 1.00 | 32.39 | 6 |
| ATOM | 10748 | O | ALA | C | 945 | 15.067 | 90.102 | 69.542 | 1.00 | 31.76 | 8 |
| ATOM | 10749 | N | ARG | C | 946 | 15.550 | 90.209 | 67.356 | 1.00 | 27.49 | 7 |
| ATOM | 10750 | CA | ARG | C | 946 | 17.002 | 90.232 | 67.528 | 1.00 | 29.39 | 6 |
| ATOM | 10751 | CB | ARG | C | 946 | 17.605 | 88.875 | 67.147 | 1.00 | 67.26 | 6 |
| ATOM | 10752 | CG | ARG | C | 946 | 19.133 | 88.814 | 67.154 | 1.00 | 69.40 | 6 |
| ATOM | 10753 | CD | ARG | C | 946 | 19.618 | 87.450 | 66.666 | 1.00 | 70.98 | 6 |
| ATOM | 10754 | NE | ARG | C | 946 | 21.073 | 87.322 | 66.608 | 1.00 | 73.54 | 7 |
| ATOM | 10755 | CZ | ARG | C | 946 | 21.887 | 88.201 | 66.030 | 1.00 | 75.59 | 6 |
| ATOM | 10756 | NH1 | ARG | C | 946 | 21.398 | 89.292 | 65.464 | 1.00 | 78.24 | 7 |
| ATOM | 10757 | NH2 | ARG | C | 946 | 23.193 | 87.972 | 65.986 | 1.00 | 76.01 | 7 |
| ATOM | 10758 | C | ARG | C | 946 | 17.558 | 91.298 | 66.613 | 1.00 | 30.19 | 6 |
| ATOM | 10759 | O | ARG | C | 946 | 18.556 | 91.915 | 66.914 | 1.00 | 30.17 | 8 |
| ATOM | 10760 | N | ALA | C | 947 | 16.903 | 91.491 | 65.480 | 1.00 | 46.59 | 7 |
| ATOM | 10761 | CA | ALA | C | 947 | 17.327 | 92.494 | 64.533 | 1.00 | 49.44 | 6 |
| ATOM | 10762 | CB | ALA | C | 947 | 16.593 | 92.324 | 63.218 | 1.00 | 95.40 | 6 |
| ATOM | 10763 | C | ALA | C | 947 | 16.963 | 93.811 | 65.167 | 1.00 | 51.92 | 6 |
| ATOM | 10764 | O | ALA | C | 947 | 17.796 | 94.709 | 65.275 | 1.00 | 53.44 | 8 |
| ATOM | 10765 | N | GLU | C | 948 | 15.714 | 93.922 | 65.608 | 1.00 | 60.56 | 7 |
| ATOM | 10766 | CA | GLU | C | 948 | 15.259 | 95.158 | 66.241 | 1.00 | 61.87 | 6 |
| ATOM | 10767 | CB | GLU | C | 948 | 13.788 | 95.059 | 66.681 | 1.00 | 79.09 | 6 |
| ATOM | 10768 | CG | GLU | C | 948 | 13.535 | 94.385 | 68.026 | 1.00 | 79.19 | 6 |
| ATOM | 10769 | CD | GLU | C | 948 | 12.111 | 94.612 | 68.520 | 1.00 | 79.55 | 6 |
| ATOM | 10770 | OE1 | GLU | C | 948 | 11.166 | 94.043 | 67.933 | 1.00 | 78.09 | 8 |
| ATOM | 10771 | OE2 | GLU | C | 948 | 11.939 | 95.375 | 69.492 | 1.00 | 80.91 | 8 |
| ATOM | 10772 | C | GLU | C | 948 | 16.152 | 95.437 | 67.441 | 1.00 | 62.71 | 6 |
| ATOM | 10773 | O | GLU | C | 948 | 16.448 | 96.593 | 67.750 | 1.00 | 63.17 | 8 |
| ATOM | 10774 | N | LYS | C | 949 | 16.582 | 94.370 | 68.111 | 1.00 | 43.00 | 7 |
| ATOM | 10775 | CA | LYS | C | 949 | 17.469 | 94.516 | 69.250 | 1.00 | 44.20 | 6 |
| ATOM | 10776 | CB | LYS | C | 949 | 17.738 | 93.164 | 69.915 | 1.00 | 150.66 | 6 |
| ATOM | 10777 | CG | LYS | C | 949 | 16.517 | 92.557 | 70.571 | 1.00 | 153.51 | 6 |
| ATOM | 10778 | CD | LYS | C | 949 | 16.856 | 91.962 | 71.925 | 1.00 | 155.35 | 6 |
| ATOM | 10779 | CE | LYS | C | 949 | 15.590 | 91.583 | 72.675 | 1.00 | 157.45 | 6 |
| ATOM | 10780 | NZ | LYS | C | 949 | 15.878 | 91.193 | 74.081 | 1.00 | 158.39 | 7 |
| ATOM | 10781 | C | LYS | C | 949 | 18.763 | 95.109 | 68.724 | 1.00 | 44.37 | 6 |
| ATOM | 10782 | O | LYS | C | 949 | 19.415 | 95.884 | 69.404 | 1.00 | 45.20 | 8 |
| ATOM | 10783 | N | LEU | C | 950 | 19.134 | 94.737 | 67.503 | 1.00 | 48.12 | 7 |
| ATOM | 10784 | CA | LEU | C | 950 | 20.341 | 95.261 | 66.889 | 1.00 | 48.09 | 6 |
| ATOM | 10785 | CB | LEU | C | 950 | 20.821 | 94.377 | 65.734 | 1.00 | 62.51 | 6 |
| ATOM | 10786 | CG | LEU | C | 950 | 21.093 | 92.870 | 65.817 | 1.00 | 64.28 | 6 |
| ATOM | 10787 | CD1 | LEU | C | 950 | 21.011 | 92.297 | 64.415 | 1.00 | 64.42 | 6 |
| ATOM | 10788 | CD2 | LEU | C | 950 | 22.451 | 92.568 | 66.436 | 1.00 | 64.51 | 6 |
| ATOM | 10789 | C | LEU | C | 950 | 19.855 | 96.554 | 66.304 | 1.00 | 47.97 | 6 |
| ATOM | 10790 | O | LEU | C | 950 | 20.254 | 96.917 | 65.224 | 1.00 | 49.44 | 8 |
| ATOM | 10791 | N | GLY | C | 951 | 18.948 | 97.230 | 66.994 | 1.00 | 44.24 | 7 |
| ATOM | 10792 | CA | GLY | C | 951 | 18.428 | 98.490 | 66.486 | 1.00 | 44.50 | 6 |
| ATOM | 10793 | C | GLY | C | 951 | 18.345 | 98.522 | 64.973 | 1.00 | 44.20 | 6 |
| ATOM | 10794 | O | GLY | C | 951 | 18.700 | 99.498 | 64.316 | 1.00 | 45.36 | 8 |
| ATOM | 10795 | N | LEU | C | 952 | 17.860 | 97.433 | 64.412 | 1.00 | 70.80 | 7 |
| ATOM | 10796 | CA | LEU | C | 952 | 17.760 | 97.328 | 62.976 | 1.00 | 70.81 | 6 |
| ATOM | 10797 | CB | LEU | C | 952 | 18.164 | 95.925 | 62.559 | 1.00 | 36.46 | 6 |
| ATOM | 10798 | CG | LEU | C | 952 | 18.891 | 95.807 | 61.242 | 1.00 | 36.82 | 6 |
| ATOM | 10799 | CD1 | LEU | C | 952 | 20.214 | 96.528 | 61.332 | 1.00 | 37.99 | 6 |
| ATOM | 10800 | CD2 | LEU | C | 952 | 19.089 | 94.354 | 60.949 | 1.00 | 36.94 | 6 |
| ATOM | 10801 | C | LEU | C | 952 | 16.336 | 97.600 | 62.535 | 1.00 | 70.39 | 6 |
| ATOM | 10802 | O | LEU | C | 952 | 16.099 | 98.135 | 61.456 | 1.00 | 70.61 | 8 |
| ATOM | 10803 | N | VAL | C | 953 | 15.392 | 97.223 | 63.385 | 1.00 | 62.37 | 7 |
| ATOM | 10804 | CA | VAL | C | 953 | 13.988 | 97.393 | 63.091 | 1.00 | 62.11 | 6 |
| ATOM | 10805 | CB | VAL | C | 953 | 13.307 | 96.031 | 63.028 | 1.00 | 71.67 | 6 |
| ATOM | 10806 | CG1 | VAL | C | 953 | 11.856 | 96.192 | 62.713 | 1.00 | 71.05 | 6 |
| ATOM | 10807 | CG2 | VAL | C | 953 | 13.974 | 95.179 | 61.985 | 1.00 | 73.42 | 6 |
| ATOM | 10808 | C | VAL | C | 953 | 13.346 | 98.221 | 64.185 | 1.00 | 62.31 | 6 |
| ATOM | 10809 | O | VAL | C | 953 | 13.832 | 98.240 | 65.317 | 1.00 | 62.52 | 8 |
| ATOM | 10810 | N | SER | C | 954 | 12.254 | 98.902 | 63.839 | 1.00 | 75.89 | 7 |
| ATOM | 10811 | CA | SER | C | 954 | 11.515 | 99.740 | 64.784 | 1.00 | 76.21 | 6 |
| ATOM | 10812 | CB | SER | C | 954 | 10.534 | 100.647 | 64.038 | 1.00 | 121.45 | 6 |
| ATOM | 10813 | OG | SER | C | 954 | 11.206 | 101.489 | 63.118 | 1.00 | 122.82 | 8 |
| ATOM | 10814 | C | SER | C | 954 | 10.742 | 98.880 | 65.778 | 1.00 | 76.00 | 6 |
| ATOM | 10815 | O | SER | C | 954 | 9.750 | 98.238 | 65.422 | 1.00 | 76.67 | 8 |
| ATOM | 10816 | N | PRO | C | 955 | 11.181 | 98.867 | 67.046 | 1.00 | 47.70 | 7 |
| ATOM | 10817 | CD | PRO | C | 955 | 12.156 | 99.785 | 67.661 | 1.00 | 36.08 | 6 |
| ATOM | 10818 | CA | PRO | C | 955 | 10.509 | 98.069 | 68.070 | 1.00 | 47.53 | 6 |
| ATOM | 10819 | CB | PRO | C | 955 | 11.396 | 98.275 | 69.285 | 1.00 | 35.83 | 6 |
| ATOM | 10820 | CG | PRO | C | 955 | 11.806 | 99.689 | 69.128 | 1.00 | 36.13 | 6 |
| ATOM | 10821 | C | PRO | C | 955 | 9.131 | 98.648 | 68.247 | 1.00 | 48.20 | 6 |
| ATOM | 10822 | O | PRO | C | 955 | 8.917 | 99.813 | 67.942 | 1.00 | 48.98 | 8 |
| ATOM | 10823 | N | GLY | C | 956 | 8.192 | 97.852 | 68.720 | 1.00 | 86.50 | 7 |
| ATOM | 10824 | CA | GLY | C | 956 | 6.860 | 98.386 | 68.889 | 1.00 | 87.54 | 6 |
| ATOM | 10825 | C | GLY | C | 956 | 5.995 | 98.058 | 67.694 | 1.00 | 87.54 | 6 |
| ATOM | 10826 | O | GLY | C | 956 | 4.778 | 97.956 | 67.818 | 1.00 | 88.81 | 8 |
| ATOM | 10827 | N | LYS | C | 957 | 6.604 | 97.914 | 66.525 | 1.00 | 49.31 | 7 |
| ATOM | 10828 | CA | LYS | C | 957 | 5.821 | 97.554 | 65.352 | 1.00 | 49.18 | 6 |
| ATOM | 10829 | CB | LYS | C | 957 | 6.596 | 97.823 | 64.057 | 1.00 | 111.66 | 6 |
| ATOM | 10830 | CG | LYS | C | 957 | 6.761 | 99.307 | 63.767 | 1.00 | 114.51 | 6 |
| ATOM | 10831 | CD | LYS | C | 957 | 7.102 | 99.596 | 62.310 | 1.00 | 115.70 | 6 |
| ATOM | 10832 | CE | LYS | C | 957 | 7.133 | 101.105 | 62.052 | 1.00 | 116.34 | 6 |
| ATOM | 10833 | NZ | LYS | C | 957 | 7.318 | 101.456 | 60.615 | 1.00 | 116.73 | 7 |
| ATOM | 10834 | C | LYS | C | 957 | 5.515 | 96.071 | 65.495 | 1.00 | 48.39 | 6 |
| ATOM | 10835 | O | LYS | C | 957 | 6.041 | 95.411 | 66.397 | 1.00 | 48.18 | 8 |
| ATOM | 10836 | N | SER | C | 958 | 4.662 | 95.548 | 64.624 | 1.00 | 49.36 | 7 |
| ATOM | 10837 | CA | SER | C | 958 | 4.292 | 94.140 | 64.682 | 1.00 | 48.83 | 6 |
| ATOM | 10838 | CB | SER | C | 958 | 3.021 | 93.918 | 63.894 | 1.00 | 39.20 | 6 |
| ATOM | 10839 | OG | SER | C | 958 | 3.321 | 93.957 | 62.515 | 1.00 | 39.36 | 8 |
| ATOM | 10840 | C | SER | C | 958 | 5.388 | 93.311 | 64.047 | 1.00 | 47.48 | 6 |
| ATOM | 10841 | O | SER | C | 958 | 6.341 | 93.854 | 63.509 | 1.00 | 48.45 | 8 |
| ATOM | 10842 | N | PRO | C | 959 | 5.278 | 91.978 | 64.099 | 1.00 | 33.17 | 7 |
| ATOM | 10843 | CD | PRO | C | 959 | 4.193 | 91.077 | 64.519 | 1.00 | 60.72 | 6 |
| ATOM | 10844 | CA | PRO | C | 959 | 6.351 | 91.224 | 63.460 | 1.00 | 32.55 | 6 |
| ATOM | 10845 | CB | PRO | C | 959 | 5.967 | 89.785 | 63.756 | 1.00 | 60.49 | 6 |
| ATOM | 10846 | CG | PRO | C | 959 | 4.470 | 89.844 | 63.691 | 1.00 | 60.58 | 6 |
| ATOM | 10847 | C | PRO | C | 959 | 6.273 | 91.549 | 61.969 | 1.00 | 32.42 | 6 |
| ATOM | 10848 | O | PRO | C | 959 | 7.193 | 92.108 | 61.382 | 1.00 | 33.33 | 8 |
| ATOM | 10849 | N | GLU | C | 960 | 5.142 | 91.211 | 61.371 | 1.00 | 28.89 | 7 |
| ATOM | 10850 | CA | GLU | C | 960 | 4.928 | 91.476 | 59.967 | 1.00 | 28.90 | 6 |
| ATOM | 10851 | CB | GLU | C | 960 | 3.441 | 91.334 | 59.651 | 1.00 | 76.30 | 6 |
| ATOM | 10852 | CG | GLU | C | 960 | 3.049 | 91.816 | 58.276 | 1.00 | 80.08 | 6 |
| ATOM | 10853 | CD | GLU | C | 960 | 1.625 | 91.448 | 57.930 | 1.00 | 82.04 | 6 |
| ATOM | 10854 | OE1 | GLU | C | 960 | 1.090 | 91.992 | 56.939 | 1.00 | 83.71 | 8 |
| ATOM | 10855 | OE2 | GLU | C | 960 | 1.042 | 90.609 | 58.649 | 1.00 | 83.11 | 8 |
| ATOM | 10856 | C | GLU | C | 960 | 5.432 | 92.859 | 59.558 | 1.00 | 27.63 | 6 |
| ATOM | 10857 | O | GLU | C | 960 | 6.114 | 92.999 | 58.551 | 1.00 | 27.27 | 8 |
| ATOM | 10858 | N | GLU | C | 961 | 5.098 | 93.880 | 60.338 | 1.00 | 43.98 | 7 |
| ATOM | 10859 | CA | GLU | C | 961 | 5.534 | 95.246 | 60.032 | 1.00 | 43.39 | 6 |
| ATOM | 10860 | CB | GLU | C | 961 | 4.912 | 96.257 | 61.009 | 1.00 | 128.06 | 6 |
| ATOM | 10861 | CG | GLU | C | 961 | 3.405 | 96.441 | 60.885 | 1.00 | 131.38 | 6 |
| ATOM | 10862 | CD | GLU | C | 961 | 2.877 | 97.558 | 61.773 | 1.00 | 132.41 | 6 |
| ATOM | 10863 | OE1 | GLU | C | 961 | 3.203 | 98.736 | 61.515 | 1.00 | 132.88 | 8 |
| ATOM | 10864 | OE2 | GLU | C | 961 | 2.136 | 97.261 | 62.732 | 1.00 | 132.88 | 8 |
| ATOM | 10865 | C | GLU | C | 961 | 7.051 | 95.391 | 60.088 | 1.00 | 41.36 | 6 |
| ATOM | 10866 | O | GLU | C | 961 | 7.615 | 96.258 | 59.422 | 1.00 | 40.70 | 8 |
| ATOM | 10867 | N | GLN | C | 962 | 7.697 | 94.547 | 60.892 | 1.00 | 54.22 | 7 |
| ATOM | 10868 | CA | GLN | C | 962 | 9.148 | 94.570 | 61.057 | 1.00 | 52.66 | 6 |
| ATOM | 10869 | CB | GLN | C | 962 | 9.525 | 93.854 | 62.358 | 1.00 | 76.65 | 6 |
| ATOM | 10870 | CG | GLN | C | 962 | 8.944 | 94.523 | 63.605 | 1.00 | 77.86 | 6 |
| ATOM | 10871 | CD | GLN | C | 962 | 9.128 | 93.703 | 64.870 | 1.00 | 79.19 | 6 |
| ATOM | 10872 | OE1 | GLN | C | 962 | 10.219 | 93.645 | 65.436 | 1.00 | 79.81 | 8 |
| ATOM | 10873 | NE2 | GLN | C | 962 | 8.056 | 93.055 | 65.315 | 1.00 | 79.30 | 7 |
| ATOM | 10874 | C | GLN | C | 962 | 9.840 | 93.922 | 59.859 | 1.00 | 51.03 | 6 |
| ATOM | 10875 | O | GLN | C | 962 | 11.014 | 94.180 | 59.572 | 1.00 | 50.46 | 8 |
| ATOM | 10876 | N | LEU | C | 963 | 9.095 | 93.078 | 59.157 | 1.00 | 50.36 | 7 |
| ATOM | 10877 | CA | LEU | C | 963 | 9.622 | 92.421 | 57.980 | 1.00 | 47.74 | 6 |
| ATOM | 10878 | CB | LEU | C | 963 | 8.699 | 91.281 | 57.533 | 1.00 | 29.07 | 6 |
| ATOM | 10879 | CG | LEU | C | 963 | 9.082 | 89.853 | 57.947 | 1.00 | 27.38 | 6 |
| ATOM | 10880 | CD1 | LEU | C | 963 | 10.346 | 89.418 | 57.215 | 1.00 | 27.54 | 6 |
| ATOM | 10881 | CD2 | LEU | C | 963 | 9.284 | 89.793 | 59.437 | 1.00 | 25.57 | 6 |
| ATOM | 10882 | C | LEU | C | 963 | 9.779 | 93.453 | 56.872 | 1.00 | 46.95 | 6 |
| ATOM | 10883 | O | LEU | C | 963 | 10.871 | 93.603 | 56.344 | 1.00 | 47.63 | 8 |
| ATOM | 10884 | N | LYS | C | 964 | 8.712 | 94.176 | 56.528 | 1.00 | 53.49 | 7 |
| ATOM | 10885 | CA | LYS | C | 964 | 8.823 | 95.183 | 55.469 | 1.00 | 52.34 | 6 |
| ATOM | 10886 | CB | LYS | C | 964 | 7.543 | 95.973 | 55.284 | 1.00 | 35.19 | 6 |
| ATOM | 10887 | CG | LYS | C | 964 | 7.530 | 96.706 | 53.961 | 1.00 | 34.10 | 6 |
| ATOM | 10888 | CD | LYS | C | 964 | 6.712 | 97.960 | 54.027 | 1.00 | 34.27 | 6 |
| ATOM | 10889 | CE | LYS | C | 964 | 5.797 | 98.098 | 52.817 | 1.00 | 34.44 | 6 |
| ATOM | 10890 | NZ | LYS | C | 964 | 4.535 | 97.281 | 52.888 | 1.00 | 32.98 | 7 |
| ATOM | 10891 | C | LYS | C | 964 | 9.911 | 96.161 | 55.840 | 1.00 | 52.66 | 6 |
| ATOM | 10892 | O | LYS | C | 964 | 10.628 | 96.682 | 54.987 | 1.00 | 52.76 | 8 |
| ATOM | 10893 | N | GLU | C | 965 | 10.013 | 96.431 | 57.129 | 1.00 | 54.32 | 7 |
| ATOM | 10894 | CA | GLU | C | 965 | 11.044 | 97.320 | 57.599 | 1.00 | 54.17 | 6 |
| ATOM | 10895 | CB | GLU | C | 965 | 10.964 | 97.413 | 59.126 | 1.00 | 111.33 | 6 |
| ATOM | 10896 | CG | GLU | C | 965 | 12.015 | 98.289 | 59.773 | 1.00 | 115.20 | 6 |
| ATOM | 10897 | CD | GLU | C | 965 | 11.470 | 99.102 | 60.928 | 1.00 | 118.03 | 6 |
| ATOM | 10898 | OE1 | GLU | C | 965 | 10.725 | 98.545 | 61.757 | 1.00 | 119.25 | 8 |
| ATOM | 10899 | OE2 | GLU | C | 965 | 11.797 | 100.303 | 61.010 | 1.00 | 120.17 | 8 |
| ATOM | 10900 | C | GLU | C | 965 | 12.357 | 96.676 | 57.128 | 1.00 | 52.71 | 6 |
| ATOM | 10901 | O | GLU | C | 965 | 13.047 | 97.203 | 56.247 | 1.00 | 52.60 | 8 |
| ATOM | 10902 | N | LEU | C | 966 | 12.654 | 95.503 | 57.682 | 1.00 | 37.42 | 7 |
| ATOM | 10903 | CA | LEU | C | 966 | 13.869 | 94.749 | 57.370 | 1.00 | 35.38 | 6 |
| ATOM | 10904 | CB | LEU | C | 966 | 13.952 | 93.571 | 58.358 | 1.00 | 17.23 | 6 |
| ATOM | 10905 | CG | LEU | C | 966 | 15.251 | 92.975 | 58.927 | 1.00 | 13.87 | 6 |
| ATOM | 10906 | CD1 | LEU | C | 966 | 15.997 | 93.982 | 59.793 | 1.00 | 13.87 | 6 |
| ATOM | 10907 | CD2 | LEU | C | 966 | 14.897 | 91.746 | 59.751 | 1.00 | 13.87 | 6 |
| ATOM | 10908 | C | LEU | C | 966 | 13.965 | 94.238 | 55.904 | 1.00 | 36.67 | 6 |
| ATOM | 10909 | O | LEU | C | 966 | 14.823 | 93.408 | 55.600 | 1.00 | 37.76 | 8 |
| ATOM | 10910 | N | PHE | C | 967 | 13.108 | 94.730 | 55.000 | 1.00 | 58.31 | 7 |
| ATOM | 10911 | CA | PHE | C | 967 | 13.112 | 94.285 | 53.589 | 1.00 | 58.86 | 6 |
| ATOM | 10912 | CB | PHE | C | 967 | 11.735 | 93.705 | 53.178 | 1.00 | 27.21 | 6 |
| ATOM | 10913 | CG | PHE | C | 967 | 11.721 | 93.011 | 51.815 | 1.00 | 21.93 | 6 |
| ATOM | 10914 | CD1 | PHE | C | 967 | 12.201 | 91.708 | 51.664 | 1.00 | 19.84 | 6 |
| ATOM | 10915 | CD2 | PHE | C | 967 | 11.231 | 93.656 | 50.683 | 1.00 | 19.36 | 6 |
| ATOM | 10916 | CE1 | PHE | C | 967 | 12.188 | 91.065 | 50.406 | 1.00 | 17.42 | 6 |
| ATOM | 10917 | CE2 | PHE | C | 967 | 11.225 | 93.007 | 49.432 | 1.00 | 17.33 | 6 |
| ATOM | 10918 | CZ | PHE | C | 967 | 11.703 | 91.714 | 49.302 | 1.00 | 14.58 | 6 |
| ATOM | 10919 | C | PHE | C | 967 | 13.476 | 95.399 | 52.614 | 1.00 | 61.43 | 6 |
| ATOM | 10920 | O | PHE | C | 967 | 13.977 | 95.129 | 51.528 | 1.00 | 62.22 | 8 |
| ATOM | 10921 | N | ASP | C | 968 | 13.200 | 96.646 | 52.979 | 1.00 | 48.15 | 7 |
| ATOM | 10922 | CA | ASP | C | 968 | 13.536 | 97.769 | 52.101 | 1.00 | 50.92 | 6 |
| ATOM | 10923 | CB | ASP | C | 968 | 12.656 | 98.972 | 52.406 | 1.00 | 71.89 | 6 |
| ATOM | 10924 | CG | ASP | C | 968 | 11.206 | 98.690 | 52.154 | 1.00 | 73.56 | 6 |
| ATOM | 10925 | OD1 | ASP | C | 968 | 10.783 | 98.696 | 50.979 | 1.00 | 73.95 | 8 |
| ATOM | 10926 | OD2 | ASP | C | 968 | 10.485 | 98.443 | 53.137 | 1.00 | 75.60 | 8 |
| ATOM | 10927 | C | ASP | C | 968 | 14.982 | 98.113 | 52.372 | 1.00 | 52.13 | 6 |
| ATOM | 10928 | O | ASP | C | 968 | 15.369 | 99.275 | 52.420 | 1.00 | 52.55 | 8 |
| ATOM | 10929 | N | LEU | C | 969 | 15.775 | 97.066 | 52.549 | 1.00 | 45.64 | 7 |
| ATOM | 10930 | CA | LEU | C | 969 | 17.186 | 97.185 | 52.853 | 1.00 | 48.17 | 6 |
| ATOM | 10931 | CB | LEU | C | 969 | 17.338 | 97.756 | 54.265 | 1.00 | 83.58 | 6 |
| ATOM | 10932 | CG | LEU | C | 969 | 18.722 | 97.909 | 54.885 | 1.00 | 86.40 | 6 |
| ATOM | 10933 | CD1 | LEU | C | 969 | 19.554 | 98.799 | 53.988 | 1.00 | 86.57 | 6 |
| ATOM | 10934 | CD2 | LEU | C | 969 | 18.604 | 98.481 | 56.301 | 1.00 | 86.42 | 6 |
| ATOM | 10935 | C | LEU | C | 969 | 17.725 | 95.762 | 52.786 | 1.00 | 49.26 | 6 |
| ATOM | 10936 | O | LEU | C | 969 | 17.904 | 95.218 | 51.701 | 1.00 | 50.41 | 8 |
| ATOM | 10937 | N | GLY | C | 970 | 17.943 | 95.159 | 53.953 | 1.00 | 138.99 | 7 |
| ATOM | 10938 | CA | GLY | C | 970 | 18.459 | 93.803 | 54.027 | 1.00 | 139.96 | 6 |
| ATOM | 10939 | C | GLY | C | 970 | 17.603 | 92.776 | 53.314 | 1.00 | 141.08 | 6 |
| ATOM | 10940 | O | GLY | C | 970 | 17.399 | 92.865 | 52.104 | 1.00 | 142.08 | 8 |
| ATOM | 10941 | N | ALA | C | 971 | 17.108 | 91.791 | 54.058 | 1.00 | 39.12 | 7 |
| ATOM | 10942 | CA | ALA | C | 971 | 16.278 | 90.750 | 53.465 | 1.00 | 39.29 | 6 |
| ATOM | 10943 | CB | ALA | C | 971 | 17.041 | 90.047 | 52.347 | 1.00 | 136.93 | 6 |
| ATOM | 10944 | C | ALA | C | 971 | 15.788 | 89.727 | 54.473 | 1.00 | 39.12 | 6 |
| ATOM | 10945 | O | ALA | C | 971 | 16.510 | 89.333 | 55.388 | 1.00 | 38.04 | 8 |
| ATOM | 10946 | N | VAL | C | 972 | 14.551 | 89.287 | 54.269 | 1.00 | 89.84 | 7 |
| ATOM | 10947 | CA | VAL | C | 972 | 13.889 | 88.321 | 55.145 | 1.00 | 90.03 | 6 |
| ATOM | 10948 | CB | VAL | C | 972 | 12.694 | 87.637 | 54.415 | 1.00 | 120.54 | 6 |
| ATOM | 10949 | CG1 | VAL | C | 972 | 12.163 | 86.465 | 55.235 | 1.00 | 121.75 | 6 |
| ATOM | 10950 | CG2 | VAL | C | 972 | 11.580 | 88.651 | 54.198 | 1.00 | 120.73 | 6 |
| ATOM | 10951 | C | VAL | C | 972 | 14.776 | 87.242 | 55.751 | 1.00 | 88.80 | 6 |
| ATOM | 10952 | O | VAL | C | 972 | 15.556 | 86.602 | 55.054 | 1.00 | 89.74 | 8 |
| ATOM | 10953 | N | VAL | C | 973 | 14.624 | 87.074 | 57.064 | 1.00 | 44.14 | 7 |
| ATOM | 10954 | CA | VAL | C | 973 | 15.336 | 86.095 | 57.867 | 1.00 | 42.44 | 6 |
| ATOM | 10955 | CB | VAL | C | 973 | 14.360 | 85.040 | 58.409 | 1.00 | 117.04 | 6 |
| ATOM | 10956 | CG1 | VAL | C | 973 | 15.125 | 83.926 | 59.105 | 1.00 | 118.69 | 6 |
| ATOM | 10957 | CG2 | VAL | C | 973 | 13.382 | 85.691 | 59.363 | 1.00 | 117.10 | 6 |
| ATOM | 10958 | C | VAL | C | 973 | 16.472 | 85.380 | 57.162 | 1.00 | 40.58 | 6 |
| ATOM | 10959 | O | VAL | C | 973 | 17.628 | 85.511 | 57.568 | 1.00 | 41.57 | 8 |
| ATOM | 10960 | N | LEU | C | 974 | 16.132 | 84.611 | 56.128 | 1.00 | 35.09 | 7 |
| ATOM | 10961 | CA | LEU | C | 974 | 17.099 | 83.863 | 55.316 | 1.00 | 33.19 | 6 |
| ATOM | 10962 | CB | LEU | C | 974 | 18.260 | 84.739 | 54.844 | 1.00 | 46.09 | 6 |
| ATOM | 10963 | CG | LEU | C | 974 | 19.219 | 83.956 | 53.951 | 1.00 | 46.48 | 6 |
| ATOM | 10964 | CD1 | LEU | C | 974 | 18.463 | 83.464 | 52.734 | 1.00 | 45.62 | 6 |
| ATOM | 10965 | CD2 | LEU | C | 974 | 20.377 | 84.821 | 53.546 | 1.00 | 47.11 | 6 |
| ATOM | 10966 | C | LEU | C | 974 | 17.677 | 82.652 | 56.000 | 1.00 | 32.57 | 6 |
| ATOM | 10967 | O | LEU | C | 974 | 18.799 | 82.667 | 56.516 | 1.00 | 32.45 | 8 |
| ATOM | 10968 | N | TYR | C | 975 | 16.890 | 81.592 | 55.965 | 1.00 | 70.40 | 7 |
| ATOM | 10969 | CA | TYR | C | 975 | 17.238 | 80.315 | 56.548 | 1.00 | 68.80 | 6 |
| ATOM | 10970 | CB | TYR | C | 975 | 16.149 | 79.325 | 56.198 | 1.00 | 27.41 | 6 |
| ATOM | 10971 | CG | TYR | C | 975 | 14.769 | 79.820 | 56.532 | 1.00 | 25.12 | 6 |
| ATOM | 10972 | CD1 | TYR | C | 975 | 13.834 | 80.059 | 55.538 | 1.00 | 24.63 | 6 |
| ATOM | 10973 | CE1 | TYR | C | 975 | 12.547 | 80.421 | 55.854 | 1.00 | 22.23 | 6 |
| ATOM | 10974 | CD2 | TYR | C | 975 | 14.378 | 79.974 | 57.852 | 1.00 | 23.45 | 6 |
| ATOM | 10975 | CE2 | TYR | C | 975 | 13.101 | 80.338 | 58.179 | 1.00 | 21.24 | 6 |
| ATOM | 10976 | CZ | TYR | C | 975 | 12.188 | 80.550 | 57.183 | 1.00 | 21.50 | 6 |
| ATOM | 10977 | OH | TYR | C | 975 | 10.893 | 80.819 | 57.539 | 1.00 | 22.82 | 8 |
| ATOM | 10978 | C | TYR | C | 975 | 18.566 | 79.796 | 56.036 | 1.00 | 69.49 | 6 |
| ATOM | 10979 | O | TYR | C | 975 | 19.305 | 80.501 | 55.352 | 1.00 | 70.73 | 8 |
| ATOM | 10980 | N | ASP | C | 976 | 18.854 | 78.546 | 56.374 | 1.00 | 19.13 | 7 |
| ATOM | 10981 | CA | ASP | C | 976 | 20.084 | 77.886 | 55.955 | 1.00 | 19.58 | 6 |
| ATOM | 10982 | CB | ASP | C | 976 | 21.166 | 78.029 | 57.039 | 1.00 | 77.17 | 6 |
| ATOM | 10983 | CG | ASP | C | 976 | 22.472 | 77.321 | 56.680 | 1.00 | 79.95 | 6 |
| ATOM | 10984 | OD1 | ASP | C | 976 | 22.873 | 77.346 | 55.495 | 1.00 | 82.04 | 8 |
| ATOM | 10985 | OD2 | ASP | C | 976 | 23.110 | 76.752 | 57.593 | 1.00 | 80.48 | 8 |
| ATOM | 10986 | C | ASP | C | 976 | 19.751 | 76.426 | 55.717 | 1.00 | 19.50 | 6 |
| ATOM | 10987 | O | ASP | C | 976 | 19.877 | 75.596 | 56.622 | 1.00 | 19.09 | 8 |
| ATOM | 10988 | N | GLY | C | 977 | 19.318 | 76.134 | 54.490 | 1.00 | 14.07 | 7 |
| ATOM | 10989 | CA | GLY | C | 977 | 18.939 | 74.784 | 54.087 | 1.00 | 14.99 | 6 |
| ATOM | 10990 | C | GLY | C | 977 | 19.825 | 73.665 | 54.582 | 1.00 | 16.05 | 6 |
| ATOM | 10991 | O | GLY | C | 977 | 19.477 | 72.492 | 54.505 | 1.00 | 13.87 | 8 |
| ATOM | 10992 | N | ARG | C | 978 | 20.969 | 74.063 | 55.105 | 1.00 | 40.37 | 7 |
| ATOM | 10993 | CA | ARG | C | 978 | 21.967 | 73.164 | 55.629 | 1.00 | 44.82 | 6 |
| ATOM | 10994 | CB | ARG | C | 978 | 23.320 | 73.874 | 55.566 | 1.00 | 103.56 | 6 |
| ATOM | 10995 | CG | ARG | C | 978 | 24.531 | 73.079 | 56.011 | 1.00 | 107.10 | 6 |
| ATOM | 10996 | CD | ARG | C | 978 | 25.659 | 74.056 | 56.305 | 1.00 | 109.68 | 6 |
| ATOM | 10997 | NE | ARG | C | 978 | 26.825 | 73.873 | 55.452 | 1.00 | 112.29 | 7 |
| ATOM | 10998 | CZ | ARG | C | 978 | 27.796 | 74.771 | 55.336 | 1.00 | 114.08 | 6 |
| ATOM | 10999 | NH1 | ARG | C | 978 | 27.726 | 75.908 | 56.014 | 1.00 | 113.40 | 7 |
| ATOM | 11000 | NH2 | ARG | C | 978 | 28.838 | 74.531 | 54.552 | 1.00 | 115.71 | 7 |
| ATOM | 11001 | C | ARG | C | 978 | 21.645 | 72.756 | 57.064 | 1.00 | 45.91 | 6 |
| ATOM | 11002 | O | ARG | C | 978 | 21.592 | 71.569 | 57.379 | 1.00 | 46.71 | 8 |
| ATOM | 11003 | N | THR | C | 979 | 21.428 | 73.743 | 57.929 | 1.00 | 85.10 | 7 |
| ATOM | 11004 | CA | THR | C | 979 | 21.145 | 73.486 | 59.340 | 1.00 | 87.17 | 6 |
| ATOM | 11005 | CB | THR | C | 979 | 21.722 | 74.600 | 60.241 | 1.00 | 202.64 | 6 |
| ATOM | 11006 | OG1 | THR | C | 979 | 23.112 | 74.788 | 59.949 | 1.00 | 204.66 | 8 |
| ATOM | 11007 | CG2 | THR | C | 979 | 21.574 | 74.228 | 61.710 | 1.00 | 203.31 | 6 |
| ATOM | 11008 | C | THR | C | 979 | 19.661 | 73.387 | 59.622 | 1.00 | 86.86 | 6 |
| ATOM | 11009 | O | THR | C | 979 | 19.244 | 72.777 | 60.603 | 1.00 | 88.03 | 8 |
| ATOM | 11010 | N | GLY | C | 980 | 18.862 | 73.991 | 58.757 | 1.00 | 45.86 | 7 |
| ATOM | 11011 | CA | GLY | C | 980 | 17.434 | 73.959 | 58.960 | 1.00 | 45.10 | 6 |
| ATOM | 11012 | C | GLY | C | 980 | 17.004 | 75.168 | 59.759 | 1.00 | 44.34 | 6 |
| ATOM | 11013 | O | GLY | C | 980 | 15.905 | 75.688 | 59.575 | 1.00 | 45.72 | 8 |
| ATOM | 11014 | N | GLU | C | 981 | 17.880 | 75.619 | 60.651 | 1.00 | 57.88 | 7 |
| ATOM | 11015 | CA | GLU | C | 981 | 17.603 | 76.777 | 61.489 | 1.00 | 56.67 | 6 |
| ATOM | 11016 | CB | GLU | C | 981 | 18.519 | 76.731 | 62.713 | 1.00 | 99.07 | 6 |
| ATOM | 11017 | CG | GLU | C | 981 | 18.151 | 75.600 | 63.676 | 1.00 | 105.10 | 6 |
| ATOM | 11018 | CD | GLU | C | 981 | 19.305 | 74.649 | 63.981 | 1.00 | 109.31 | 6 |
| ATOM | 11019 | OE1 | GLU | C | 981 | 20.321 | 75.095 | 64.560 | 1.00 | 112.69 | 8 |
| ATOM | 11020 | OE2 | GLU | C | 981 | 19.192 | 73.448 | 63.649 | 1.00 | 110.76 | 8 |
| ATOM | 11021 | C | GLU | C | 981 | 17.778 | 78.079 | 60.697 | 1.00 | 54.05 | 6 |
| ATOM | 11022 | O | GLU | C | 981 | 17.895 | 78.055 | 59.470 | 1.00 | 55.73 | 8 |
| ATOM | 11023 | N | PRO | C | 982 | 17.749 | 79.236 | 61.377 | 1.00 | 32.50 | 7 |
| ATOM | 11024 | CD | PRO | C | 982 | 17.006 | 79.485 | 62.627 | 1.00 | 44.67 | 6 |
| ATOM | 11025 | CA | PRO | C | 982 | 17.918 | 80.492 | 60.636 | 1.00 | 29.63 | 6 |
| ATOM | 11026 | CB | PRO | C | 982 | 16.764 | 81.331 | 61.147 | 1.00 | 43.45 | 6 |
| ATOM | 11027 | CG | PRO | C | 982 | 16.787 | 80.998 | 62.594 | 1.00 | 43.65 | 6 |
| ATOM | 11028 | C | PRO | C | 982 | 19.254 | 81.201 | 60.851 | 1.00 | 25.71 | 6 |
| ATOM | 11029 | O | PRO | C | 982 | 20.107 | 80.765 | 61.624 | 1.00 | 25.17 | 8 |
| ATOM | 11030 | N | PHE | C | 983 | 19.423 | 82.308 | 60.147 | 1.00 | 31.10 | 7 |
| ATOM | 11031 | CA | PHE | C | 983 | 20.628 | 83.107 | 60.277 | 1.00 | 28.85 | 6 |
| ATOM | 11032 | CB | PHE | C | 983 | 21.142 | 83.524 | 58.901 | 1.00 | 32.18 | 6 |
| ATOM | 11033 | CG | PHE | C | 983 | 22.035 | 82.513 | 58.259 | 1.00 | 31.04 | 6 |
| ATOM | 11034 | CD1 | PHE | C | 983 | 22.059 | 82.370 | 56.878 | 1.00 | 29.66 | 6 |
| ATOM | 11035 | CD2 | PHE | C | 983 | 22.845 | 81.697 | 59.034 | 1.00 | 31.27 | 6 |
| ATOM | 11036 | CE1 | PHE | C | 983 | 22.868 | 81.431 | 56.281 | 1.00 | 29.81 | 6 |
| ATOM | 11037 | CE2 | PHE | C | 983 | 23.664 | 80.749 | 58.439 | 1.00 | 31.41 | 6 |
| ATOM | 11038 | CZ | PHE | C | 983 | 23.673 | 80.616 | 57.058 | 1.00 | 31.06 | 6 |
| ATOM | 11039 | C | PHE | C | 983 | 20.301 | 84.349 | 61.100 | 1.00 | 27.01 | 6 |
| ATOM | 11040 | O | PHE | C | 983 | 19.869 | 85.368 | 60.555 | 1.00 | 25.65 | 8 |
| ATOM | 11041 | N | GLU | C | 984 | 20.502 | 84.247 | 62.411 | 1.00 | 37.40 | 7 |
| ATOM | 11042 | CA | GLU | C | 984 | 20.237 | 85.346 | 63.323 | 1.00 | 36.01 | 6 |
| ATOM | 11043 | CB | GLU | C | 984 | 21.034 | 85.162 | 64.605 | 1.00 | 55.68 | 6 |
| ATOM | 11044 | CG | GLU | C | 984 | 22.491 | 84.885 | 64.350 | 1.00 | 59.64 | 6 |
| ATOM | 11045 | CD | GLU | C | 984 | 22.957 | 83.626 | 65.038 | 1.00 | 62.38 | 6 |
| ATOM | 11046 | OE1 | GLU | C | 984 | 22.263 | 82.599 | 64.897 | 1.00 | 62.80 | 8 |
| ATOM | 11047 | OE2 | GLU | C | 984 | 24.012 | 83.655 | 65.713 | 1.00 | 64.81 | 8 |
| ATOM | 11048 | C | GLU | C | 984 | 20.664 | 86.617 | 62.638 | 1.00 | 33.82 | 6 |
| ATOM | 11049 | O | GLU | C | 984 | 21.764 | 86.684 | 62.100 | 1.00 | 34.00 | 8 |
| ATOM | 11050 | N | GLY | C | 985 | 19.791 | 87.617 | 62.643 | 1.00 | 30.99 | 7 |
| ATOM | 11051 | CA | GLY | C | 985 | 20.117 | 88.876 | 61.997 | 1.00 | 28.15 | 6 |
| ATOM | 11052 | C | GLY | C | 985 | 19.962 | 88.772 | 60.495 | 1.00 | 26.16 | 6 |
| ATOM | 11053 | O | GLY | C | 985 | 20.641 | 87.974 | 59.849 | 1.00 | 26.05 | 8 |
| ATOM | 11054 | N | PRO | C | 986 | 19.074 | 89.581 | 59.907 | 1.00 | 29.03 | 7 |
| ATOM | 11055 | CD | PRO | C | 986 | 18.449 | 90.779 | 60.484 | 1.00 | 15.25 | 6 |
| ATOM | 11056 | CA | PRO | C | 986 | 18.867 | 89.531 | 58.460 | 1.00 | 27.60 | 6 |
| ATOM | 11057 | CB | PRO | C | 986 | 18.174 | 90.857 | 58.156 | 1.00 | 14.69 | 6 |
| ATOM | 11058 | CG | PRO | C | 986 | 18.516 | 91.723 | 59.336 | 1.00 | 14.93 | 6 |
| ATOM | 11059 | C | PRO | C | 986 | 20.165 | 89.354 | 57.700 | 1.00 | 26.72 | 6 |
| ATOM | 11060 | O | PRO | C | 986 | 21.248 | 89.466 | 58.281 | 1.00 | 27.37 | 8 |
| ATOM | 11061 | N | ILE | C | 987 | 20.038 | 89.077 | 56.400 | 1.00 | 18.42 | 7 |
| ATOM | 11062 | CA | ILE | C | 987 | 21.171 | 88.845 | 55.501 | 1.00 | 16.80 | 6 |
| ATOM | 11063 | CB | ILE | C | 987 | 21.441 | 87.323 | 55.385 | 1.00 | 13.87 | 6 |
| ATOM | 11064 | CG2 | ILE | C | 987 | 22.381 | 87.047 | 54.266 | 1.00 | 13.94 | 6 |
| ATOM | 11065 | CG1 | ILE | C | 987 | 22.057 | 86.786 | 56.676 | 1.00 | 13.87 | 6 |
| ATOM | 11066 | CD1 | ILE | C | 987 | 23.462 | 87.322 | 56.970 | 1.00 | 16.12 | 6 |
| ATOM | 11067 | C | ILE | C | 987 | 20.831 | 89.385 | 54.124 | 1.00 | 16.80 | 6 |
| ATOM | 11068 | O | ILE | C | 987 | 20.304 | 88.650 | 53.322 | 1.00 | 16.80 | 8 |
| ATOM | 11069 | N | VAL | C | 988 | 21.139 | 90.645 | 53.832 | 1.00 | 47.22 | 7 |
| ATOM | 11070 | CA | VAL | C | 988 | 20.786 | 91.234 | 52.525 | 1.00 | 49.47 | 6 |
| ATOM | 11071 | CB | VAL | C | 988 | 21.558 | 92.571 | 52.279 | 1.00 | 16.80 | 6 |
| ATOM | 11072 | CG1 | VAL | C | 988 | 23.000 | 92.417 | 52.664 | 1.00 | 17.34 | 6 |
| ATOM | 11073 | CG2 | VAL | C | 988 | 21.424 | 92.994 | 50.839 | 1.00 | 15.58 | 6 |
| ATOM | 11074 | C | VAL | C | 988 | 20.930 | 90.312 | 51.302 | 1.00 | 50.17 | 6 |
| ATOM | 11075 | O | VAL | C | 988 | 21.991 | 89.747 | 51.053 | 1.00 | 52.27 | 8 |
| ATOM | 11076 | N | VAL | C | 989 | 19.845 | 90.175 | 50.544 | 1.00 | 24.68 | 7 |
| ATOM | 11077 | CA | VAL | C | 989 | 19.807 | 89.308 | 49.368 | 1.00 | 25.07 | 6 |
| ATOM | 11078 | CB | VAL | C | 989 | 18.705 | 88.229 | 49.554 | 1.00 | 14.32 | 6 |
| ATOM | 11079 | CG1 | VAL | C | 989 | 18.403 | 87.536 | 48.255 | 1.00 | 17.13 | 6 |
| ATOM | 11080 | CG2 | VAL | C | 989 | 19.147 | 87.216 | 50.570 | 1.00 | 15.03 | 6 |
| ATOM | 11081 | C | VAL | C | 989 | 19.526 | 90.130 | 48.102 | 1.00 | 26.60 | 6 |
| ATOM | 11082 | O | VAL | C | 989 | 19.168 | 91.304 | 48.200 | 1.00 | 28.55 | 8 |
| ATOM | 11083 | N | GLY | C | 990 | 19.697 | 89.526 | 46.924 | 1.00 | 30.67 | 7 |
| ATOM | 11084 | CA | GLY | C | 990 | 19.438 | 90.234 | 45.677 | 1.00 | 31.43 | 6 |
| ATOM | 11085 | C | GLY | C | 990 | 20.007 | 89.601 | 44.409 | 1.00 | 31.89 | 6 |
| ATOM | 11086 | O | GLY | C | 990 | 20.667 | 88.568 | 44.455 | 1.00 | 31.21 | 8 |
| ATOM | 11087 | N | GLN | C | 991 | 19.737 | 90.216 | 43.261 | 1.00 | 17.81 | 7 |
| ATOM | 11088 | CA | GLN | C | 991 | 20.248 | 89.709 | 41.993 | 1.00 | 18.72 | 6 |
| ATOM | 11089 | CB | GLN | C | 991 | 19.194 | 89.772 | 40.896 | 1.00 | 44.96 | 6 |
| ATOM | 11090 | CG | GLN | C | 991 | 17.821 | 89.294 | 41.246 | 1.00 | 48.05 | 6 |
| ATOM | 11091 | CD | GLN | C | 991 | 17.051 | 88.956 | 39.991 | 1.00 | 49.93 | 6 |
| ATOM | 11092 | OE1 | GLN | C | 991 | 16.981 | 89.759 | 39.054 | 1.00 | 49.34 | 8 |
| ATOM | 11093 | NE2 | GLN | C | 991 | 16.479 | 87.761 | 39.955 | 1.00 | 50.17 | 7 |
| ATOM | 11094 | C | GLN | C | 991 | 21.416 | 90.553 | 41.518 | 1.00 | 18.07 | 6 |
| ATOM | 11095 | O | GLN | C | 991 | 21.221 | 91.573 | 40.866 | 1.00 | 17.99 | 8 |
| ATOM | 11096 | N | MET | C | 992 | 22.638 | 90.156 | 41.824 | 1.00 | 39.09 | 7 |
| ATOM | 11097 | CA | MET | C | 992 | 23.745 | 90.963 | 41.339 | 1.00 | 38.59 | 6 |
| ATOM | 11098 | CB | MET | C | 992 | 25.077 | 90.544 | 41.976 | 1.00 | 20.30 | 6 |
| ATOM | 11099 | CG | MET | C | 992 | 26.120 | 91.636 | 41.928 | 1.00 | 17.71 | 6 |
| ATOM | 11100 | SD | MET | C | 992 | 27.194 | 91.687 | 43.389 | 1.00 | 20.68 | 16 |
| ATOM | 11101 | CE | MET | C | 992 | 26.016 | 91.680 | 44.760 | 1.00 | 15.97 | 6 |
| ATOM | 11102 | C | MET | C | 992 | 23.791 | 90.771 | 39.831 | 1.00 | 39.94 | 6 |
| ATOM | 11103 | O | MET | C | 992 | 22.813 | 91.031 | 39.134 | 1.00 | 40.95 | 8 |
| ATOM | 11104 | N | PHE | C | 993 | 24.921 | 90.283 | 39.341 | 1.00 | 44.24 | 7 |
| ATOM | 11105 | CA | PHE | C | 993 | 25.129 | 90.067 | 37.914 | 1.00 | 44.14 | 6 |
| ATOM | 11106 | CB | PHE | C | 993 | 24.714 | 91.328 | 37.144 | 1.00 | 27.79 | 6 |
| ATOM | 11107 | CG | PHE | C | 993 | 25.320 | 91.456 | 35.774 | 1.00 | 27.42 | 6 |
| ATOM | 11108 | CD1 | PHE | C | 993 | 26.553 | 92.070 | 35.601 | 1.00 | 27.20 | 6 |
| ATOM | 11109 | CD2 | PHE | C | 993 | 24.628 | 91.025 | 34.651 | 1.00 | 27.04 | 6 |
| ATOM | 11110 | CE1 | PHE | C | 993 | 27.081 | 92.258 | 34.330 | 1.00 | 26.20 | 6 |
| ATOM | 11111 | CE2 | PHE | C | 993 | 25.150 | 91.210 | 33.378 | 1.00 | 26.64 | 6 |
| ATOM | 11112 | CZ | PHE | C | 993 | 26.375 | 91.828 | 33.217 | 1.00 | 26.08 | 6 |
| ATOM | 11113 | C | PHE | C | 993 | 26.617 | 89.809 | 37.789 | 1.00 | 44.72 | 6 |
| ATOM | 11114 | O | PHE | C | 993 | 27.413 | 90.742 | 37.730 | 1.00 | 45.45 | 8 |
| ATOM | 11115 | N | ILE | C | 994 | 26.995 | 88.541 | 37.780 | 1.00 | 44.61 | 7 |
| ATOM | 11116 | CA | ILE | C | 994 | 28.399 | 88.204 | 37.697 | 1.00 | 43.80 | 6 |
| ATOM | 11117 | CB | ILE | C | 994 | 28.784 | 87.142 | 38.737 | 1.00 | 19.13 | 6 |
| ATOM | 11118 | CG2 | ILE | C | 994 | 30.207 | 87.334 | 39.090 | 1.00 | 20.66 | 6 |
| ATOM | 11119 | CG1 | ILE | C | 994 | 27.911 | 87.231 | 40.000 | 1.00 | 17.49 | 6 |
| ATOM | 11120 | CD1 | ILE | C | 994 | 27.914 | 88.565 | 40.693 | 1.00 | 17.51 | 6 |
| ATOM | 11121 | C | ILE | C | 994 | 28.784 | 87.677 | 36.322 | 1.00 | 43.75 | 6 |
| ATOM | 11122 | O | ILE | C | 994 | 28.221 | 86.688 | 35.846 | 1.00 | 43.06 | 8 |
| ATOM | 11123 | N | MET | C | 995 | 29.746 | 88.337 | 35.686 | 1.00 | 37.58 | 7 |
| ATOM | 11124 | CA | MET | C | 995 | 30.210 | 87.924 | 34.370 | 1.00 | 35.68 | 6 |
| ATOM | 11125 | CB | MET | C | 995 | 30.945 | 89.076 | 33.705 | 1.00 | 20.97 | 6 |
| ATOM | 11126 | CG | MET | C | 995 | 30.322 | 90.428 | 33.981 | 1.00 | 20.58 | 6 |
| ATOM | 11127 | SD | MET | C | 995 | 31.466 | 91.803 | 33.727 | 1.00 | 21.18 | 16 |
| ATOM | 11128 | CE | MET | C | 995 | 30.986 | 92.311 | 32.174 | 1.00 | 22.42 | 6 |
| ATOM | 11129 | C | MET | C | 995 | 31.171 | 86.784 | 34.648 | 1.00 | 36.47 | 6 |
| ATOM | 11130 | O | MET | C | 995 | 31.370 | 86.430 | 35.801 | 1.00 | 36.94 | 8 |
| ATOM | 11131 | N | LYS | C | 996 | 31.753 | 86.198 | 33.609 | 1.00 | 46.96 | 7 |
| ATOM | 11132 | CA | LYS | C | 996 | 32.717 | 85.108 | 33.790 | 1.00 | 47.84 | 6 |
| ATOM | 11133 | CB | LYS | C | 996 | 32.073 | 83.757 | 33.477 | 1.00 | 17.54 | 6 |
| ATOM | 11134 | CG | LYS | C | 996 | 33.056 | 82.621 | 33.376 | 1.00 | 13.87 | 6 |
| ATOM | 11135 | CD | LYS | C | 996 | 32.376 | 81.391 | 32.847 | 1.00 | 13.87 | 6 |
| ATOM | 11136 | CE | LYS | C | 996 | 33.385 | 80.444 | 32.215 | 1.00 | 13.87 | 6 |
| ATOM | 11137 | NZ | LYS | C | 996 | 32.800 | 79.143 | 31.773 | 1.00 | 13.87 | 7 |
| ATOM | 11138 | C | LYS | C | 996 | 33.904 | 85.345 | 32.860 | 1.00 | 51.11 | 6 |
| ATOM | 11139 | O | LYS | C | 996 | 33.804 | 85.125 | 31.658 | 1.00 | 53.25 | 8 |
| ATOM | 11140 | N | LEU | C | 997 | 35.025 | 85.784 | 33.426 | 1.00 | 15.42 | 7 |
| ATOM | 11141 | CA | LEU | C | 997 | 36.225 | 86.095 | 32.664 | 1.00 | 15.70 | 6 |
| ATOM | 11142 | CB | LEU | C | 997 | 37.174 | 86.840 | 33.569 | 1.00 | 13.87 | 6 |
| ATOM | 11143 | CG | LEU | C | 997 | 36.487 | 88.046 | 34.168 | 1.00 | 13.87 | 6 |
| ATOM | 11144 | CD1 | LEU | C | 997 | 37.351 | 88.663 | 35.226 | 1.00 | 13.87 | 6 |
| ATOM | 11145 | CD2 | LEU | C | 997 | 36.187 | 89.013 | 33.090 | 1.00 | 13.87 | 6 |
| ATOM | 11146 | C | LEU | C | 997 | 36.968 | 84.958 | 31.953 | 1.00 | 17.74 | 6 |
| ATOM | 11147 | O | LEU | C | 997 | 36.949 | 83.808 | 32.368 | 1.00 | 18.03 | 8 |
| ATOM | 11148 | N | TYR | C | 998 | 37.645 | 85.322 | 30.873 | 1.00 | 20.72 | 7 |
| ATOM | 11149 | CA | TYR | C | 998 | 38.408 | 84.407 | 30.035 | 1.00 | 24.37 | 6 |
| ATOM | 11150 | CB | TYR | C | 998 | 39.214 | 85.242 | 29.023 | 1.00 | 92.26 | 6 |
| ATOM | 11151 | CG | TYR | C | 998 | 40.008 | 84.460 | 27.990 | 1.00 | 96.60 | 6 |
| ATOM | 11152 | CD1 | TYR | C | 998 | 39.480 | 83.321 | 27.372 | 1.00 | 98.30 | 6 |
| ATOM | 11153 | CE1 | TYR | C | 998 | 40.209 | 82.619 | 26.397 | 1.00 | 98.92 | 6 |
| ATOM | 11154 | CD2 | TYR | C | 998 | 41.283 | 84.880 | 27.607 | 1.00 | 98.51 | 6 |
| ATOM | 11155 | CE2 | TYR | C | 998 | 42.016 | 84.190 | 26.633 | 1.00 | 98.97 | 6 |
| ATOM | 11156 | CZ | TYR | C | 998 | 41.476 | 83.064 | 26.035 | 1.00 | 98.33 | 6 |
| ATOM | 11157 | OH | TYR | C | 998 | 42.207 | 82.396 | 25.077 | 1.00 | 98.89 | 8 |
| ATOM | 11158 | C | TYR | C | 998 | 39.314 | 83.378 | 30.727 | 1.00 | 25.75 | 6 |
| ATOM | 11159 | O | TYR | C | 998 | 39.751 | 82.435 | 30.071 | 1.00 | 27.14 | 8 |
| ATOM | 11160 | N | HIS | C | 999 | 39.584 | 83.507 | 32.028 | 1.00 | 41.06 | 7 |
| ATOM | 11161 | CA | HIS | C | 999 | 40.466 | 82.532 | 32.694 | 1.00 | 43.13 | 6 |
| ATOM | 11162 | CB | HIS | C | 999 | 40.904 | 83.059 | 34.055 | 1.00 | 74.02 | 6 |
| ATOM | 11163 | CG | HIS | C | 999 | 42.100 | 83.956 | 33.975 | 1.00 | 78.41 | 6 |
| ATOM | 11164 | CD2 | HIS | C | 999 | 42.389 | 84.978 | 33.136 | 1.00 | 79.36 | 6 |
| ATOM | 11165 | ND1 | HIS | C | 999 | 43.190 | 83.824 | 34.812 | 1.00 | 80.62 | 7 |
| ATOM | 11166 | CE1 | HIS | C | 999 | 44.100 | 84.727 | 34.488 | 1.00 | 80.65 | 6 |
| ATOM | 11167 | NE2 | HIS | C | 999 | 43.640 | 85.440 | 33.475 | 1.00 | 80.99 | 7 |
| ATOM | 11168 | C | HIS | C | 999 | 40.014 | 81.061 | 32.827 | 1.00 | 42.37 | 6 |
| ATOM | 11169 | O | HIS | C | 999 | 40.634 | 80.165 | 32.249 | 1.00 | 42.56 | 8 |
| ATOM | 11170 | N | MET | C | 1000 | 38.956 | 80.808 | 33.590 | 1.00 | 61.43 | 7 |
| ATOM | 11171 | CA | MET | C | 1000 | 38.420 | 79.451 | 33.775 | 1.00 | 60.85 | 6 |
| ATOM | 11172 | CB | MET | C | 1000 | 37.354 | 79.165 | 32.710 | 1.00 | 57.57 | 6 |
| ATOM | 11173 | CG | MET | C | 1000 | 37.804 | 79.398 | 31.273 | 1.00 | 61.63 | 6 |
| ATOM | 11174 | SD | MET | C | 1000 | 36.405 | 79.619 | 30.147 | 1.00 | 64.55 | 16 |
| ATOM | 11175 | CE | MET | C | 1000 | 35.399 | 78.247 | 30.678 | 1.00 | 65.80 | 6 |
| ATOM | 11176 | C | MET | C | 1000 | 39.415 | 78.287 | 33.832 | 1.00 | 59.77 | 6 |
| ATOM | 11177 | O | MET | C | 1000 | 39.571 | 77.541 | 32.875 | 1.00 | 60.85 | 8 |
| ATOM | 11178 | N | VAL | C | 1001 | 40.055 | 78.153 | 34.990 | 1.00 | 16.94 | 7 |
| ATOM | 11179 | CA | VAL | C | 1001 | 41.032 | 77.119 | 35.336 | 1.00 | 14.11 | 6 |
| ATOM | 11180 | CB | VAL | C | 1001 | 40.466 | 76.118 | 36.340 | 1.00 | 38.93 | 6 |
| ATOM | 11181 | CG1 | VAL | C | 1001 | 41.562 | 75.158 | 36.736 | 1.00 | 39.27 | 6 |
| ATOM | 11182 | CG2 | VAL | C | 1001 | 39.887 | 76.836 | 37.553 | 1.00 | 38.97 | 6 |
| ATOM | 11183 | C | VAL | C | 1001 | 41.623 | 76.283 | 34.242 | 1.00 | 14.44 | 6 |
| ATOM | 11184 | O | VAL | C | 1001 | 42.806 | 76.408 | 33.954 | 1.00 | 14.38 | 8 |
| ATOM | 11185 | N | GLU | C | 1002 | 40.794 | 75.397 | 33.683 | 1.00 | 60.65 | 7 |
| ATOM | 11186 | CA | GLU | C | 1002 | 41.168 | 74.466 | 32.614 | 1.00 | 62.16 | 6 |
| ATOM | 11187 | CB | GLU | C | 1002 | 40.051 | 74.334 | 31.573 | 1.00 | 72.07 | 6 |
| ATOM | 11188 | CG | GLU | C | 1002 | 38.731 | 73.790 | 32.072 | 1.00 | 75.81 | 6 |
| ATOM | 11189 | CD | GLU | C | 1002 | 37.791 | 73.451 | 30.931 | 1.00 | 79.01 | 6 |
| ATOM | 11190 | OE1 | GLU | C | 1002 | 38.059 | 72.470 | 30.208 | 1.00 | 81.12 | 8 |
| ATOM | 11191 | OE2 | GLU | C | 1002 | 36.792 | 74.176 | 30.743 | 1.00 | 80.37 | 8 |
| ATOM | 11192 | C | GLU | C | 1002 | 42.427 | 74.840 | 31.870 | 1.00 | 60.50 | 6 |
| ATOM | 11193 | O | GLU | C | 1002 | 43.363 | 74.050 | 31.789 | 1.00 | 60.82 | 8 |
| ATOM | 11194 | N | ASP | C | 1003 | 42.432 | 76.046 | 31.314 | 1.00 | 26.50 | 7 |
| ATOM | 11195 | CA | ASP | C | 1003 | 43.557 | 76.539 | 30.544 | 1.00 | 24.70 | 6 |
| ATOM | 11196 | CB | ASP | C | 1003 | 43.171 | 77.863 | 29.923 | 1.00 | 50.89 | 6 |
| ATOM | 11197 | CG | ASP | C | 1003 | 41.850 | 77.778 | 29.203 | 1.00 | 52.83 | 6 |
| ATOM | 11198 | OD1 | ASP | C | 1003 | 41.358 | 78.817 | 28.722 | 1.00 | 54.56 | 8 |
| ATOM | 11199 | OD2 | ASP | C | 1003 | 41.298 | 76.661 | 29.120 | 1.00 | 53.96 | 8 |
| ATOM | 11200 | C | ASP | C | 1003 | 44.828 | 76.657 | 31.369 | 1.00 | 23.02 | 6 |
| ATOM | 11201 | O | ASP | C | 1003 | 45.929 | 76.641 | 30.828 | 1.00 | 22.80 | 8 |
| ATOM | 11202 | N | LYS | C | 1004 | 44.665 | 76.773 | 32.680 | 1.00 | 19.96 | 7 |
| ATOM | 11203 | CA | LYS | C | 1004 | 45.784 | 76.827 | 33.599 | 1.00 | 19.04 | 6 |
| ATOM | 11204 | CB | LYS | C | 1004 | 45.507 | 77.803 | 34.730 | 1.00 | 20.56 | 6 |
| ATOM | 11205 | CG | LYS | C | 1004 | 44.694 | 79.018 | 34.353 | 1.00 | 23.05 | 6 |
| ATOM | 11206 | CD | LYS | C | 1004 | 45.453 | 79.942 | 33.422 | 1.00 | 24.51 | 6 |
| ATOM | 11207 | CE | LYS | C | 1004 | 44.811 | 81.320 | 33.408 | 1.00 | 25.76 | 6 |
| ATOM | 11208 | NZ | LYS | C | 1004 | 45.465 | 82.237 | 32.435 | 1.00 | 28.59 | 7 |
| ATOM | 11209 | C | LYS | C | 1004 | 45.798 | 75.409 | 34.176 | 1.00 | 19.56 | 6 |
| ATOM | 11210 | O | LYS | C | 1004 | 44.875 | 74.628 | 33.931 | 1.00 | 20.55 | 8 |
| ATOM | 11211 | N | MET | C | 1005 | 46.826 | 75.077 | 34.953 | 1.00 | 42.15 | 7 |
| ATOM | 11212 | CA | MET | C | 1005 | 46.944 | 73.753 | 35.570 | 1.00 | 41.06 | 6 |
| ATOM | 11213 | CB | MET | C | 1005 | 45.607 | 73.299 | 36.187 | 1.00 | 20.69 | 6 |
| ATOM | 11214 | CG | MET | C | 1005 | 45.116 | 71.887 | 35.804 | 1.00 | 20.20 | 6 |
| ATOM | 11215 | SD | MET | C | 1005 | 45.128 | 70.556 | 37.078 | 1.00 | 20.38 | 16 |
| ATOM | 11216 | CE | MET | C | 1005 | 46.148 | 69.418 | 36.337 | 1.00 | 19.70 | 6 |
| ATOM | 11217 | C | MET | C | 1005 | 47.436 | 72.676 | 34.632 | 1.00 | 42.58 | 6 |
| ATOM | 11218 | O | MET | C | 1005 | 46.715 | 72.223 | 33.749 | 1.00 | 44.78 | 8 |
| ATOM | 11219 | N | HIS | C | 1006 | 48.689 | 72.293 | 34.816 | 1.00 | 13.87 | 7 |
| ATOM | 11220 | CA | HIS | C | 1006 | 49.280 | 71.215 | 34.065 | 1.00 | 13.87 | 6 |
| ATOM | 11221 | CB | HIS | C | 1006 | 50.369 | 71.735 | 33.126 | 1.00 | 93.10 | 6 |
| ATOM | 11222 | CG | HIS | C | 1006 | 50.334 | 71.102 | 31.766 | 1.00 | 99.57 | 6 |
| ATOM | 11223 | CD2 | HIS | C | 1006 | 49.583 | 70.085 | 31.277 | 1.00 | 100.85 | 6 |
| ATOM | 11224 | ND1 | HIS | C | 1006 | 51.140 | 71.516 | 30.725 | 1.00 | 102.52 | 7 |
| ATOM | 11225 | CE1 | HIS | C | 1006 | 50.885 | 70.782 | 29.655 | 1.00 | 102.71 | 6 |
| ATOM | 11226 | NE2 | HIS | C | 1006 | 49.945 | 69.907 | 29.963 | 1.00 | 102.00 | 7 |
| ATOM | 11227 | C | HIS | C | 1006 | 49.844 | 70.404 | 35.224 | 1.00 | 13.87 | 6 |
| ATOM | 11228 | O | HIS | C | 1006 | 49.484 | 70.650 | 36.358 | 1.00 | 13.87 | 8 |
| ATOM | 11229 | N | ALA | C | 1007 | 50.698 | 69.433 | 34.976 | 1.00 | 33.72 | 7 |
| ATOM | 11230 | CA | ALA | C | 1007 | 51.249 | 68.662 | 36.079 | 1.00 | 34.73 | 6 |
| ATOM | 11231 | CB | ALA | C | 1007 | 50.153 | 67.947 | 36.820 | 1.00 | 13.87 | 6 |
| ATOM | 11232 | C | ALA | C | 1007 | 52.241 | 67.668 | 35.524 | 1.00 | 36.05 | 6 |
| ATOM | 11233 | O | ALA | C | 1007 | 51.867 | 66.701 | 34.866 | 1.00 | 36.02 | 8 |
| ATOM | 11234 | N | ARG | C | 1008 | 53.517 | 67.916 | 35.780 | 1.00 | 41.26 | 7 |
| ATOM | 11235 | CA | ARG | C | 1008 | 54.565 | 67.043 | 35.281 | 1.00 | 43.17 | 6 |
| ATOM | 11236 | CB | ARG | C | 1008 | 55.689 | 67.878 | 34.668 | 1.00 | 33.91 | 6 |
| ATOM | 11237 | CG | ARG | C | 1008 | 56.866 | 67.078 | 34.181 | 1.00 | 32.25 | 6 |
| ATOM | 11238 | CD | ARG | C | 1008 | 57.486 | 67.733 | 32.969 | 1.00 | 30.96 | 6 |
| ATOM | 11239 | NE | ARG | C | 1008 | 58.791 | 67.162 | 32.682 | 1.00 | 30.64 | 7 |
| ATOM | 11240 | CZ | ARG | C | 1008 | 59.493 | 67.393 | 31.583 | 1.00 | 29.97 | 6 |
| ATOM | 11241 | NH1 | ARG | C | 1008 | 59.026 | 68.193 | 30.635 | 1.00 | 29.22 | 7 |
| ATOM | 11242 | NH2 | ARG | C | 1008 | 60.676 | 66.818 | 31.440 | 1.00 | 30.67 | 7 |
| ATOM | 11243 | C | ARG | C | 1008 | 55.128 | 66.132 | 36.347 | 1.00 | 45.10 | 6 |
| ATOM | 11244 | O | ARG | C | 1008 | 55.051 | 66.416 | 37.541 | 1.00 | 45.89 | 8 |
| ATOM | 11245 | N | SER | C | 1009 | 55.687 | 65.022 | 35.895 | 1.00 | 49.92 | 7 |
| ATOM | 11246 | CA | SER | C | 1009 | 56.292 | 64.056 | 36.784 | 1.00 | 51.93 | 6 |
| ATOM | 11247 | CB | SER | C | 1009 | 55.375 | 62.861 | 36.983 | 1.00 | 57.98 | 6 |
| ATOM | 11248 | OG | SER | C | 1009 | 55.872 | 62.041 | 38.021 | 1.00 | 59.37 | 8 |
| ATOM | 11249 | C | SER | C | 1009 | 57.547 | 63.617 | 36.080 | 1.00 | 52.84 | 6 |
| ATOM | 11250 | O | SER | C | 1009 | 58.581 | 63.409 | 36.706 | 1.00 | 53.28 | 8 |
| ATOM | 11251 | N | THR | C | 1010 | 57.439 | 63.477 | 34.763 | 1.00 | 27.93 | 7 |
| ATOM | 11252 | CA | THR | C | 1010 | 58.573 | 63.087 | 33.948 | 1.00 | 29.04 | 6 |
| ATOM | 11253 | CB | THR | C | 1010 | 58.727 | 61.561 | 33.885 | 1.00 | 37.56 | 6 |
| ATOM | 11254 | OG1 | THR | C | 1010 | 58.409 | 60.985 | 35.157 | 1.00 | 36.45 | 8 |
| ATOM | 11255 | CG2 | THR | C | 1010 | 60.165 | 61.205 | 33.559 | 1.00 | 37.90 | 6 |
| ATOM | 11256 | C | THR | C | 1010 | 58.391 | 63.637 | 32.540 | 1.00 | 30.66 | 6 |
| ATOM | 11257 | O | THR | C | 1010 | 58.680 | 64.800 | 32.282 | 1.00 | 30.57 | 8 |
| ATOM | 11258 | N | GLY | C | 1011 | 57.928 | 62.799 | 31.623 | 1.00 | 42.65 | 7 |
| ATOM | 11259 | CA | GLY | C | 1011 | 57.694 | 63.247 | 30.255 | 1.00 | 45.25 | 6 |
| ATOM | 11260 | C | GLY | C | 1011 | 58.796 | 63.964 | 29.489 | 1.00 | 46.79 | 6 |
| ATOM | 11261 | O | GLY | C | 1011 | 59.914 | 64.085 | 29.967 | 1.00 | 47.00 | 8 |
| ATOM | 11262 | N | PRO | C | 1012 | 58.479 | 64.479 | 28.291 | 1.00 | 48.83 | 7 |
| ATOM | 11263 | CD | PRO | C | 1012 | 57.064 | 64.689 | 27.931 | 1.00 | 114.49 | 6 |
| ATOM | 11264 | CA | PRO | C | 1012 | 59.342 | 65.201 | 27.352 | 1.00 | 49.93 | 6 |
| ATOM | 11265 | CB | PRO | C | 1012 | 58.376 | 66.173 | 26.700 | 1.00 | 114.23 | 6 |
| ATOM | 11266 | CG | PRO | C | 1012 | 57.158 | 65.353 | 26.572 | 1.00 | 114.37 | 6 |
| ATOM | 11267 | C | PRO | C | 1012 | 60.568 | 65.914 | 27.901 | 1.00 | 51.27 | 6 |
| ATOM | 11268 | O | PRO | C | 1012 | 60.460 | 66.788 | 28.759 | 1.00 | 51.89 | 8 |
| ATOM | 11269 | N | TYR | C | 1013 | 61.728 | 65.554 | 27.363 | 1.00 | 86.75 | 7 |
| ATOM | 11270 | CA | TYR | C | 1013 | 63.019 | 66.141 | 27.745 | 1.00 | 87.75 | 6 |
| ATOM | 11271 | CB | TYR | C | 1013 | 63.976 | 65.016 | 28.111 | 1.00 | 53.54 | 6 |
| ATOM | 11272 | CG | TYR | C | 1013 | 64.625 | 65.034 | 29.482 | 1.00 | 53.45 | 6 |
| ATOM | 11273 | CD1 | TYR | C | 1013 | 63.866 | 64.966 | 30.650 | 1.00 | 52.95 | 6 |
| ATOM | 11274 | CE1 | TYR | C | 1013 | 64.482 | 64.779 | 31.886 | 1.00 | 52.43 | 6 |
| ATOM | 11275 | CD2 | TYR | C | 1013 | 66.020 | 64.937 | 29.594 | 1.00 | 53.96 | 6 |
| ATOM | 11276 | CE2 | TYR | C | 1013 | 66.640 | 64.753 | 30.808 | 1.00 | 53.24 | 6 |
| ATOM | 11277 | CZ | TYR | C | 1013 | 65.875 | 64.667 | 31.946 | 1.00 | 52.45 | 6 |
| ATOM | 11278 | OH | TYR | C | 1013 | 66.521 | 64.425 | 33.131 | 1.00 | 52.61 | 8 |
| ATOM | 11279 | C | TYR | C | 1013 | 63.537 | 66.857 | 26.478 | 1.00 | 88.24 | 6 |
| ATOM | 11280 | O | TYR | C | 1013 | 63.463 | 66.291 | 25.378 | 1.00 | 89.30 | 8 |
| ATOM | 11281 | N | SER | C | 1014 | 64.069 | 68.072 | 26.610 | 1.00 | 47.08 | 7 |
| ATOM | 11282 | CA | SER | C | 1014 | 64.545 | 68.794 | 25.427 | 1.00 | 47.39 | 6 |
| ATOM | 11283 | CB | SER | C | 1014 | 64.378 | 70.292 | 25.598 | 1.00 | 60.42 | 6 |
| ATOM | 11284 | OG | SER | C | 1014 | 65.170 | 70.966 | 24.627 | 1.00 | 63.02 | 8 |
| ATOM | 11285 | C | SER | C | 1014 | 65.983 | 68.546 | 25.002 | 1.00 | 47.75 | 6 |
| ATOM | 11286 | O | SER | C | 1014 | 66.906 | 68.585 | 25.820 | 1.00 | 47.83 | 8 |
| ATOM | 11287 | N | LEU | C | 1015 | 66.170 | 68.342 | 23.699 | 1.00 | 70.61 | 7 |
| ATOM | 11288 | CA | LEU | C | 1015 | 67.491 | 68.071 | 23.150 | 1.00 | 69.98 | 6 |
| ATOM | 11289 | CB | LEU | C | 1015 | 67.386 | 67.105 | 21.969 | 1.00 | 114.54 | 6 |
| ATOM | 11290 | CG | LEU | C | 1015 | 66.451 | 67.451 | 20.811 | 1.00 | 115.93 | 6 |
| ATOM | 11291 | CD1 | LEU | C | 1015 | 66.940 | 68.690 | 20.084 | 1.00 | 116.00 | 6 |
| ATOM | 11292 | CD2 | LEU | C | 1015 | 66.392 | 66.264 | 19.860 | 1.00 | 116.40 | 6 |
| ATOM | 11293 | C | LEU | C | 1015 | 68.296 | 69.287 | 22.738 | 1.00 | 68.72 | 6 |
| ATOM | 11294 | O | LEU | C | 1015 | 69.349 | 69.139 | 22.133 | 1.00 | 68.77 | 8 |
| ATOM | 11295 | N | ILE | C | 1016 | 67.803 | 70.484 | 23.039 | 1.00 | 37.53 | 7 |
| ATOM | 11296 | CA | ILE | C | 1016 | 68.548 | 71.699 | 22.718 | 1.00 | 36.23 | 6 |
| ATOM | 11297 | CB | ILE | C | 1016 | 67.667 | 72.807 | 22.170 | 1.00 | 50.00 | 6 |
| ATOM | 11298 | CG2 | ILE | C | 1016 | 68.508 | 74.065 | 21.950 | 1.00 | 48.92 | 6 |
| ATOM | 11299 | CG1 | ILE | C | 1016 | 67.011 | 72.346 | 20.875 | 1.00 | 49.95 | 6 |
| ATOM | 11300 | CD1 | ILE | C | 1016 | 67.986 | 72.089 | 19.768 | 1.00 | 50.95 | 6 |
| ATOM | 11301 | C | ILE | C | 1016 | 69.152 | 72.224 | 24.001 | 1.00 | 35.96 | 6 |
| ATOM | 11302 | O | ILE | C | 1016 | 70.359 | 72.441 | 24.083 | 1.00 | 35.90 | 8 |
| ATOM | 11303 | N | THR | C | 1017 | 68.292 | 72.443 | 24.994 | 1.00 | 39.57 | 7 |
| ATOM | 11304 | CA | THR | C | 1017 | 68.722 | 72.929 | 26.300 | 1.00 | 39.57 | 6 |
| ATOM | 11305 | CB | THR | C | 1017 | 67.583 | 73.655 | 27.052 | 1.00 | 88.65 | 6 |
| ATOM | 11306 | OG1 | THR | C | 1017 | 66.860 | 74.501 | 26.146 | 1.00 | 88.92 | 8 |
| ATOM | 11307 | CG2 | THR | C | 1017 | 68.162 | 74.509 | 28.171 | 1.00 | 89.88 | 6 |
| ATOM | 11308 | C | THR | C | 1017 | 69.097 | 71.676 | 27.068 | 1.00 | 39.20 | 6 |
| ATOM | 11309 | O | THR | C | 1017 | 70.265 | 71.333 | 27.164 | 1.00 | 38.71 | 8 |
| ATOM | 11310 | N | GLN | C | 1018 | 68.085 | 71.002 | 27.600 | 1.00 | 17.95 | 7 |
| ATOM | 11311 | CA | GLN | C | 1018 | 68.233 | 69.745 | 28.331 | 1.00 | 18.99 | 6 |
| ATOM | 11312 | CB | GLN | C | 1018 | 69.688 | 69.452 | 28.670 | 1.00 | 56.47 | 6 |
| ATOM | 11313 | CG | GLN | C | 1018 | 70.209 | 68.246 | 27.920 | 1.00 | 58.39 | 6 |
| ATOM | 11314 | CD | GLN | C | 1018 | 69.214 | 67.107 | 27.933 | 1.00 | 58.71 | 6 |
| ATOM | 11315 | OE1 | GLN | C | 1018 | 68.140 | 67.206 | 27.351 | 1.00 | 58.37 | 8 |
| ATOM | 11316 | NE2 | GLN | C | 1018 | 69.565 | 66.020 | 28.601 | 1.00 | 59.16 | 7 |
| ATOM | 11317 | C | GLN | C | 1018 | 67.398 | 69.628 | 29.591 | 1.00 | 19.88 | 6 |
| ATOM | 11318 | O | GLN | C | 1018 | 67.473 | 68.624 | 30.312 | 1.00 | 18.83 | 8 |
| ATOM | 11319 | N | GLN | C | 1019 | 66.603 | 70.658 | 29.847 | 1.00 | 47.41 | 7 |
| ATOM | 11320 | CA | GLN | C | 1019 | 65.716 | 70.670 | 30.990 | 1.00 | 50.89 | 6 |
| ATOM | 11321 | CB | GLN | C | 1019 | 65.710 | 72.060 | 31.623 | 1.00 | 49.09 | 6 |
| ATOM | 11322 | CG | GLN | C | 1019 | 66.269 | 73.139 | 30.730 | 1.00 | 48.74 | 6 |
| ATOM | 11323 | CD | GLN | C | 1019 | 66.348 | 74.477 | 31.428 | 1.00 | 48.54 | 6 |
| ATOM | 11324 | OE1 | GLN | C | 1019 | 66.829 | 74.574 | 32.554 | 1.00 | 49.01 | 8 |
| ATOM | 11325 | NE2 | GLN | C | 1019 | 65.889 | 75.522 | 30.757 | 1.00 | 48.79 | 7 |
| ATOM | 11326 | C | GLN | C | 1019 | 64.308 | 70.280 | 30.513 | 1.00 | 53.88 | 6 |
| ATOM | 11327 | O | GLN | C | 1019 | 64.002 | 70.346 | 29.323 | 1.00 | 54.77 | 8 |
| ATOM | 11328 | N | PRO | C | 1020 | 63.443 | 69.843 | 31.438 | 1.00 | 44.35 | 7 |
| ATOM | 11329 | CD | PRO | C | 1020 | 63.750 | 69.666 | 32.869 | 1.00 | 98.24 | 6 |
| ATOM | 11330 | CA | PRO | C | 1020 | 62.066 | 69.434 | 31.157 | 1.00 | 47.24 | 6 |
| ATOM | 11331 | CB | PRO | C | 1020 | 61.458 | 69.356 | 32.551 | 1.00 | 99.76 | 6 |
| ATOM | 11332 | CG | PRO | C | 1020 | 62.584 | 68.832 | 33.353 | 1.00 | 99.02 | 6 |
| ATOM | 11333 | C | PRO | C | 1020 | 61.314 | 70.419 | 30.265 | 1.00 | 50.54 | 6 |
| ATOM | 11334 | O | PRO | C | 1020 | 61.434 | 71.626 | 30.451 | 1.00 | 49.81 | 8 |
| ATOM | 11335 | N | LEU | C | 1021 | 60.545 | 69.896 | 29.310 | 1.00 | 94.66 | 7 |
| ATOM | 11336 | CA | LEU | C | 1021 | 59.742 | 70.709 | 28.392 | 1.00 | 99.47 | 6 |
| ATOM | 11337 | CB | LEU | C | 1021 | 60.335 | 72.115 | 28.205 | 1.00 | 184.73 | 6 |
| ATOM | 11338 | CG | LEU | C | 1021 | 59.764 | 73.255 | 29.060 | 1.00 | 186.62 | 6 |
| ATOM | 11339 | CD1 | LEU | C | 1021 | 60.683 | 74.459 | 28.957 | 1.00 | 186.97 | 6 |
| ATOM | 11340 | CD2 | LEU | C | 1021 | 58.350 | 73.609 | 28.609 | 1.00 | 188.57 | 6 |
| ATOM | 11341 | C | LEU | C | 1021 | 59.570 | 70.069 | 27.025 | 1.00 | 102.04 | 6 |
| ATOM | 11342 | O | LEU | C | 1021 | 59.666 | 68.854 | 26.884 | 1.00 | 102.75 | 8 |
| ATOM | 11343 | N | GLY | C | 1022 | 59.305 | 70.916 | 26.033 | 1.00 | 87.07 | 7 |
| ATOM | 11344 | CA | GLY | C | 1022 | 59.107 | 70.488 | 24.657 | 1.00 | 90.28 | 6 |
| ATOM | 11345 | C | GLY | C | 1022 | 59.074 | 68.998 | 24.379 | 1.00 | 92.33 | 6 |
| ATOM | 11346 | O | GLY | C | 1022 | 60.107 | 68.329 | 24.406 | 1.00 | 92.40 | 8 |
| ATOM | 11347 | N | GLY | C | 1023 | 57.882 | 68.480 | 24.094 | 1.00 | 208.87 | 7 |
| ATOM | 11348 | CA | GLY | C | 1023 | 57.740 | 67.063 | 23.806 | 1.00 | 208.87 | 6 |
| ATOM | 11349 | C | GLY | C | 1023 | 56.633 | 66.740 | 22.822 | 1.00 | 208.87 | 6 |
| ATOM | 11350 | O | GLY | C | 1023 | 56.423 | 67.473 | 21.853 | 1.00 | 208.87 | 8 |
| ATOM | 11351 | N | LYS | C | 1024 | 55.919 | 65.643 | 23.069 | 1.00 | 154.54 | 7 |
| ATOM | 11352 | CA | LYS | C | 1024 | 54.839 | 65.221 | 22.186 | 1.00 | 155.10 | 6 |
| ATOM | 11353 | CB | LYS | C | 1024 | 55.418 | 64.465 | 20.983 | 1.00 | 208.87 | 6 |
| ATOM | 11354 | CG | LYS | C | 1024 | 56.416 | 65.266 | 20.150 | 1.00 | 208.87 | 6 |
| ATOM | 11355 | CD | LYS | C | 1024 | 57.177 | 64.393 | 19.159 | 1.00 | 208.87 | 6 |
| ATOM | 11356 | CE | LYS | C | 1024 | 58.222 | 65.204 | 18.400 | 1.00 | 208.87 | 6 |
| ATOM | 11357 | NZ | LYS | C | 1024 | 59.015 | 64.369 | 17.456 | 1.00 | 208.87 | 7 |
| ATOM | 11358 | C | LYS | C | 1024 | 53.819 | 64.332 | 22.895 | 1.00 | 154.31 | 6 |
| ATOM | 11359 | O | LYS | C | 1024 | 52.613 | 64.492 | 22.705 | 1.00 | 154.61 | 8 |
| ATOM | 11360 | N | ALA | C | 1025 | 54.310 | 63.400 | 23.710 | 1.00 | 98.15 | 7 |
| ATOM | 11361 | CA | ALA | C | 1025 | 53.453 | 62.453 | 24.432 | 1.00 | 96.34 | 6 |
| ATOM | 11362 | CB | ALA | C | 1025 | 54.306 | 61.318 | 24.994 | 1.00 | 167.23 | 6 |
| ATOM | 11363 | C | ALA | C | 1025 | 52.634 | 63.091 | 25.550 | 1.00 | 94.94 | 6 |
| ATOM | 11364 | O | ALA | C | 1025 | 51.509 | 63.544 | 25.335 | 1.00 | 93.58 | 8 |
| ATOM | 11365 | N | GLN | C | 1026 | 53.190 | 63.079 | 26.755 | 1.00 | 132.87 | 7 |
| ATOM | 11366 | CA | GLN | C | 1026 | 52.532 | 63.691 | 27.897 | 1.00 | 132.40 | 6 |
| ATOM | 11367 | CB | GLN | C | 1026 | 53.036 | 63.076 | 29.204 | 1.00 | 208.77 | 6 |
| ATOM | 11368 | CG | GLN | C | 1026 | 52.752 | 61.586 | 29.357 | 1.00 | 208.87 | 6 |
| ATOM | 11369 | CD | GLN | C | 1026 | 53.571 | 60.719 | 28.414 | 1.00 | 208.87 | 6 |
| ATOM | 11370 | OE1 | GLN | C | 1026 | 54.801 | 60.698 | 28.479 | 1.00 | 208.87 | 8 |
| ATOM | 11371 | NE2 | GLN | C | 1026 | 52.889 | 59.995 | 27.534 | 1.00 | 208.87 | 7 |
| ATOM | 11372 | C | GLN | C | 1026 | 52.975 | 65.138 | 27.800 | 1.00 | 131.14 | 6 |
| ATOM | 11373 | O | GLN | C | 1026 | 53.337 | 65.595 | 26.716 | 1.00 | 131.60 | 8 |
| ATOM | 11374 | N | PHE | C | 1027 | 52.966 | 65.860 | 28.913 | 1.00 | 82.92 | 7 |
| ATOM | 11375 | CA | PHE | C | 1027 | 53.403 | 67.250 | 28.877 | 1.00 | 80.71 | 6 |
| ATOM | 11376 | CB | PHE | C | 1027 | 52.459 | 68.074 | 27.998 | 1.00 | 208.87 | 6 |
| ATOM | 11377 | CG | PHE | C | 1027 | 53.164 | 68.885 | 26.947 | 1.00 | 208.87 | 6 |
| ATOM | 11378 | CD1 | PHE | C | 1027 | 54.015 | 68.271 | 26.032 | 1.00 | 208.87 | 6 |
| ATOM | 11379 | CD2 | PHE | C | 1027 | 52.973 | 70.261 | 26.864 | 1.00 | 208.87 | 6 |
| ATOM | 11380 | CE1 | PHE | C | 1027 | 54.665 | 69.013 | 25.052 | 1.00 | 208.87 | 6 |
| ATOM | 11381 | CE2 | PHE | C | 1027 | 53.620 | 71.014 | 25.884 | 1.00 | 208.87 | 6 |
| ATOM | 11382 | CZ | PHE | C | 1027 | 54.467 | 70.387 | 24.976 | 1.00 | 208.87 | 6 |
| ATOM | 11383 | C | PHE | C | 1027 | 53.508 | 67.887 | 30.255 | 1.00 | 77.67 | 6 |
| ATOM | 11384 | O | PHE | C | 1027 | 52.813 | 67.496 | 31.195 | 1.00 | 77.36 | 8 |
| ATOM | 11385 | N | GLY | C | 1028 | 54.389 | 68.873 | 30.369 | 1.00 | 49.95 | 7 |
| ATOM | 11386 | CA | GLY | C | 1028 | 54.552 | 69.539 | 31.642 | 1.00 | 46.68 | 6 |
| ATOM | 11387 | C | GLY | C | 1028 | 55.682 | 70.545 | 31.700 | 1.00 | 43.63 | 6 |
| ATOM | 11388 | O | GLY | C | 1028 | 55.707 | 71.511 | 30.943 | 1.00 | 44.95 | 8 |
| ATOM | 11389 | N | GLY | C | 1029 | 56.620 | 70.313 | 32.608 | 1.00 | 66.31 | 7 |
| ATOM | 11390 | CA | GLY | C | 1029 | 57.730 | 71.223 | 32.762 | 1.00 | 63.47 | 6 |
| ATOM | 11391 | C | GLY | C | 1029 | 57.277 | 72.286 | 33.730 | 1.00 | 61.22 | 6 |
| ATOM | 11392 | O | GLY | C | 1029 | 56.098 | 72.607 | 33.764 | 1.00 | 62.75 | 8 |
| ATOM | 11393 | N | GLN | C | 1030 | 58.193 | 72.824 | 34.525 | 1.00 | 81.20 | 7 |
| ATOM | 11394 | CA | GLN | C | 1030 | 57.850 | 73.854 | 35.496 | 1.00 | 78.10 | 6 |
| ATOM | 11395 | CB | GLN | C | 1030 | 57.485 | 73.210 | 36.816 | 1.00 | 31.57 | 6 |
| ATOM | 11396 | CG | GLN | C | 1030 | 56.815 | 74.167 | 37.728 | 1.00 | 28.64 | 6 |
| ATOM | 11397 | CD | GLN | C | 1030 | 55.739 | 74.943 | 37.028 | 1.00 | 25.08 | 6 |
| ATOM | 11398 | OE1 | GLN | C | 1030 | 54.896 | 74.372 | 36.352 | 1.00 | 21.41 | 8 |
| ATOM | 11399 | NE2 | GLN | C | 1030 | 55.756 | 76.257 | 37.189 | 1.00 | 26.95 | 7 |
| ATOM | 11400 | C | GLN | C | 1030 | 59.050 | 74.755 | 35.689 | 1.00 | 77.82 | 6 |
| ATOM | 11401 | O | GLN | C | 1030 | 60.161 | 74.351 | 35.381 | 1.00 | 79.19 | 8 |
| ATOM | 11402 | N | ARG | C | 1031 | 58.856 | 75.963 | 36.205 | 1.00 | 45.25 | 7 |
| ATOM | 11403 | CA | ARG | C | 1031 | 60.001 | 76.849 | 36.373 | 1.00 | 44.23 | 6 |
| ATOM | 11404 | CB | ARG | C | 1031 | 59.735 | 78.199 | 35.702 | 1.00 | 110.67 | 6 |
| ATOM | 11405 | CG | ARG | C | 1031 | 59.607 | 79.394 | 36.643 | 1.00 | 113.90 | 6 |
| ATOM | 11406 | CD | ARG | C | 1031 | 58.163 | 79.634 | 37.099 | 1.00 | 115.04 | 6 |
| ATOM | 11407 | NE | ARG | C | 1031 | 57.921 | 81.056 | 37.355 | 1.00 | 115.65 | 7 |
| ATOM | 11408 | CZ | ARG | C | 1031 | 56.733 | 81.584 | 37.635 | 1.00 | 116.14 | 6 |
| ATOM | 11409 | NH1 | ARG | C | 1031 | 55.653 | 80.814 | 37.705 | 1.00 | 116.14 | 7 |
| ATOM | 11410 | NH2 | ARG | C | 1031 | 56.628 | 82.890 | 37.833 | 1.00 | 116.64 | 7 |
| ATOM | 11411 | C | ARG | C | 1031 | 60.443 | 77.055 | 37.809 | 1.00 | 41.69 | 6 |
| ATOM | 11412 | O | ARG | C | 1031 | 59.641 | 76.984 | 38.736 | 1.00 | 42.41 | 8 |
| ATOM | 11413 | N | PHE | C | 1032 | 61.734 | 77.327 | 37.981 | 1.00 | 40.03 | 7 |
| ATOM | 11414 | CA | PHE | C | 1032 | 62.296 | 77.530 | 39.308 | 1.00 | 37.15 | 6 |
| ATOM | 11415 | CB | PHE | C | 1032 | 63.197 | 76.363 | 39.645 | 1.00 | 20.08 | 6 |
| ATOM | 11416 | CG | PHE | C | 1032 | 63.291 | 76.088 | 41.105 | 1.00 | 15.12 | 6 |
| ATOM | 11417 | CD1 | PHE | C | 1032 | 62.133 | 75.992 | 41.875 | 1.00 | 13.87 | 6 |
| ATOM | 11418 | CD2 | PHE | C | 1032 | 64.528 | 75.902 | 41.709 | 1.00 | 13.87 | 6 |
| ATOM | 11419 | CE1 | PHE | C | 1032 | 62.200 | 75.714 | 43.217 | 1.00 | 13.87 | 6 |
| ATOM | 11420 | CE2 | PHE | C | 1032 | 64.608 | 75.626 | 43.041 | 1.00 | 13.87 | 6 |
| ATOM | 11421 | CZ | PHE | C | 1032 | 63.435 | 75.530 | 43.806 | 1.00 | 13.87 | 6 |
| ATOM | 11422 | C | PHE | C | 1032 | 63.076 | 78.835 | 39.467 | 1.00 | 37.93 | 6 |
| ATOM | 11423 | O | PHE | C | 1032 | 64.089 | 78.882 | 40.163 | 1.00 | 38.84 | 8 |
| ATOM | 11424 | N | GLY | C | 1033 | 62.577 | 79.895 | 38.830 | 1.00 | 61.10 | 7 |
| ATOM | 11425 | CA | GLY | C | 1033 | 63.213 | 81.203 | 38.884 | 1.00 | 59.25 | 6 |
| ATOM | 11426 | C | GLY | C | 1033 | 63.779 | 81.651 | 40.218 | 1.00 | 57.87 | 6 |
| ATOM | 11427 | O | GLY | C | 1033 | 63.054 | 81.901 | 41.170 | 1.00 | 58.37 | 8 |
| ATOM | 11428 | N | GLU | C | 1034 | 65.097 | 81.752 | 40.260 | 1.00 | 35.29 | 7 |
| ATOM | 11429 | CA | GLU | C | 1034 | 65.857 | 82.182 | 41.424 | 1.00 | 33.00 | 6 |
| ATOM | 11430 | CB | GLU | C | 1034 | 66.677 | 83.414 | 41.060 | 1.00 | 27.87 | 6 |
| ATOM | 11431 | CG | GLU | C | 1034 | 65.836 | 84.585 | 40.637 | 1.00 | 26.20 | 6 |
| ATOM | 11432 | CD | GLU | C | 1034 | 66.580 | 85.890 | 40.726 | 1.00 | 24.84 | 6 |
| ATOM | 11433 | OE1 | GLU | C | 1034 | 67.141 | 86.180 | 41.807 | 1.00 | 25.48 | 8 |
| ATOM | 11434 | OE2 | GLU | C | 1034 | 66.593 | 86.629 | 39.720 | 1.00 | 22.96 | 8 |
| ATOM | 11435 | C | GLU | C | 1034 | 65.140 | 82.462 | 42.741 | 1.00 | 32.41 | 6 |
| ATOM | 11436 | O | GLU | C | 1034 | 65.570 | 81.964 | 43.774 | 1.00 | 33.50 | 8 |
| ATOM | 11437 | N | MET | C | 1035 | 64.091 | 83.285 | 42.724 | 1.00 | 33.04 | 7 |
| ATOM | 11438 | CA | MET | C | 1035 | 63.328 | 83.639 | 43.942 | 1.00 | 32.68 | 6 |
| ATOM | 11439 | CB | MET | C | 1035 | 61.990 | 84.263 | 43.553 | 1.00 | 45.39 | 6 |
| ATOM | 11440 | CG | MET | C | 1035 | 62.119 | 85.470 | 42.667 | 1.00 | 47.77 | 6 |
| ATOM | 11441 | SD | MET | C | 1035 | 62.423 | 86.954 | 43.583 | 1.00 | 49.69 | 16 |
| ATOM | 11442 | CE | MET | C | 1035 | 60.892 | 87.829 | 43.198 | 1.00 | 49.06 | 6 |
| ATOM | 11443 | C | MET | C | 1035 | 63.048 | 82.441 | 44.853 | 1.00 | 31.66 | 6 |
| ATOM | 11444 | O | MET | C | 1035 | 63.073 | 82.546 | 46.086 | 1.00 | 30.23 | 8 |
| ATOM | 11445 | N | GLU | C | 1036 | 62.747 | 81.317 | 44.214 | 1.00 | 43.75 | 7 |
| ATOM | 11446 | CA | GLU | C | 1036 | 62.471 | 80.068 | 44.891 | 1.00 | 42.00 | 6 |
| ATOM | 11447 | CB | GLU | C | 1036 | 61.799 | 79.098 | 43.919 | 1.00 | 70.48 | 6 |
| ATOM | 11448 | CG | GLU | C | 1036 | 60.411 | 79.552 | 43.437 | 1.00 | 73.46 | 6 |
| ATOM | 11449 | CD | GLU | C | 1036 | 60.437 | 80.813 | 42.572 | 1.00 | 74.77 | 6 |
| ATOM | 11450 | OE1 | GLU | C | 1036 | 60.961 | 80.756 | 41.442 | 1.00 | 74.81 | 8 |
| ATOM | 11451 | OE2 | GLU | C | 1036 | 59.927 | 81.860 | 43.021 | 1.00 | 76.12 | 8 |
| ATOM | 11452 | C | GLU | C | 1036 | 63.801 | 79.505 | 45.381 | 1.00 | 40.42 | 6 |
| ATOM | 11453 | O | GLU | C | 1036 | 63.846 | 78.777 | 46.369 | 1.00 | 40.09 | 8 |
| ATOM | 11454 | N | VAL | C | 1037 | 64.886 | 79.852 | 44.687 | 1.00 | 51.83 | 7 |
| ATOM | 11455 | CA | VAL | C | 1037 | 66.232 | 79.413 | 45.070 | 1.00 | 50.11 | 6 |
| ATOM | 11456 | CB | VAL | C | 1037 | 67.305 | 79.845 | 44.058 | 1.00 | 31.64 | 6 |
| ATOM | 11457 | CG1 | VAL | C | 1037 | 68.654 | 79.316 | 44.500 | 1.00 | 30.33 | 6 |
| ATOM | 11458 | CG2 | VAL | C | 1037 | 66.957 | 79.341 | 42.684 | 1.00 | 33.01 | 6 |
| ATOM | 11459 | C | VAL | C | 1037 | 66.594 | 80.054 | 46.405 | 1.00 | 50.34 | 6 |
| ATOM | 11460 | O | VAL | C | 1037 | 67.065 | 79.373 | 47.318 | 1.00 | 51.42 | 8 |
| ATOM | 11461 | N | TRP | C | 1038 | 66.406 | 81.371 | 46.504 | 1.00 | 31.86 | 7 |
| ATOM | 11462 | CA | TRP | C | 1038 | 66.680 | 82.053 | 47.752 | 1.00 | 30.02 | 6 |
| ATOM | 11463 | CB | TRP | C | 1038 | 66.274 | 83.524 | 47.691 | 1.00 | 29.88 | 6 |
| ATOM | 11464 | CG | TRP | C | 1038 | 67.065 | 84.403 | 46.768 | 1.00 | 30.43 | 6 |
| ATOM | 11465 | CD2 | TRP | C | 1038 | 66.622 | 85.636 | 46.170 | 1.00 | 30.63 | 6 |
| ATOM | 11466 | CE2 | TRP | C | 1038 | 67.688 | 86.135 | 45.405 | 1.00 | 30.04 | 6 |
| ATOM | 11467 | CE3 | TRP | C | 1038 | 65.422 | 86.360 | 46.207 | 1.00 | 30.95 | 6 |
| ATOM | 11468 | CD1 | TRP | C | 1038 | 68.342 | 84.219 | 46.357 | 1.00 | 30.76 | 6 |
| ATOM | 11469 | NE1 | TRP | C | 1038 | 68.730 | 85.256 | 45.536 | 1.00 | 30.48 | 7 |
| ATOM | 11470 | CZ2 | TRP | C | 1038 | 67.596 | 87.317 | 44.681 | 1.00 | 29.69 | 6 |
| ATOM | 11471 | CZ3 | TRP | C | 1038 | 65.333 | 87.531 | 45.489 | 1.00 | 29.39 | 6 |
| ATOM | 11472 | CH2 | TRP | C | 1038 | 66.411 | 87.998 | 44.736 | 1.00 | 28.83 | 6 |
| ATOM | 11473 | C | TRP | C | 1038 | 65.806 | 81.353 | 48.796 | 1.00 | 28.68 | 6 |
| ATOM | 11474 | O | TRP | C | 1038 | 66.289 | 80.948 | 49.844 | 1.00 | 28.13 | 8 |
| ATOM | 11475 | N | ALA | C | 1039 | 64.515 | 81.215 | 48.499 | 1.00 | 18.31 | 7 |
| ATOM | 11476 | CA | ALA | C | 1039 | 63.565 | 80.558 | 49.399 | 1.00 | 17.79 | 6 |
| ATOM | 11477 | CB | ALA | C | 1039 | 62.329 | 80.121 | 48.633 | 1.00 | 62.41 | 6 |
| ATOM | 11478 | C | ALA | C | 1039 | 64.200 | 79.360 | 50.054 | 1.00 | 17.24 | 6 |
| ATOM | 11479 | O | ALA | C | 1039 | 64.203 | 79.259 | 51.270 | 1.00 | 16.36 | 8 |
| ATOM | 11480 | N | LEU | C | 1040 | 64.740 | 78.452 | 49.244 | 1.00 | 14.18 | 7 |
| ATOM | 11481 | CA | LEU | C | 1040 | 65.391 | 77.254 | 49.776 | 1.00 | 13.87 | 6 |
| ATOM | 11482 | CB | LEU | C | 1040 | 65.697 | 76.246 | 48.657 | 1.00 | 40.62 | 6 |
| ATOM | 11483 | CG | LEU | C | 1040 | 64.594 | 75.316 | 48.107 | 1.00 | 42.47 | 6 |
| ATOM | 11484 | CD1 | LEU | C | 1040 | 63.908 | 74.560 | 49.245 | 1.00 | 42.97 | 6 |
| ATOM | 11485 | CD2 | LEU | C | 1040 | 63.579 | 76.119 | 47.341 | 1.00 | 42.23 | 6 |
| ATOM | 11486 | C | LEU | C | 1040 | 66.669 | 77.590 | 50.543 | 1.00 | 13.87 | 6 |
| ATOM | 11487 | O | LEU | C | 1040 | 66.893 | 77.069 | 51.630 | 1.00 | 13.87 | 8 |
| ATOM | 11488 | N | GLU | C | 1041 | 67.506 | 78.457 | 49.976 | 1.00 | 29.26 | 7 |
| ATOM | 11489 | CA | GLU | C | 1041 | 68.733 | 78.857 | 50.655 | 1.00 | 28.23 | 6 |
| ATOM | 11490 | CB | GLU | C | 1041 | 69.457 | 80.000 | 49.901 | 1.00 | 19.32 | 6 |
| ATOM | 11491 | CG | GLU | C | 1041 | 69.804 | 79.664 | 48.436 | 1.00 | 19.62 | 6 |
| ATOM | 11492 | CD | GLU | C | 1041 | 71.042 | 80.396 | 47.870 | 1.00 | 20.88 | 6 |
| ATOM | 11493 | OE1 | GLU | C | 1041 | 71.017 | 81.622 | 47.594 | 1.00 | 18.20 | 8 |
| ATOM | 11494 | OE2 | GLU | C | 1041 | 72.064 | 79.713 | 47.684 | 1.00 | 22.50 | 8 |
| ATOM | 11495 | C | GLU | C | 1041 | 68.280 | 79.319 | 52.040 | 1.00 | 27.64 | 6 |
| ATOM | 11496 | O | GLU | C | 1041 | 68.595 | 78.686 | 53.043 | 1.00 | 27.24 | 8 |
| ATOM | 11497 | N | ALA | C | 1042 | 67.494 | 80.391 | 52.078 | 1.00 | 15.12 | 7 |
| ATOM | 11498 | CA | ALA | C | 1042 | 66.985 | 80.964 | 53.320 | 1.00 | 15.86 | 6 |
| ATOM | 11499 | CB | ALA | C | 1042 | 65.865 | 81.938 | 53.021 | 1.00 | 42.81 | 6 |
| ATOM | 11500 | C | ALA | C | 1042 | 66.496 | 79.894 | 54.274 | 1.00 | 17.96 | 6 |
| ATOM | 11501 | O | ALA | C | 1042 | 66.644 | 80.020 | 55.495 | 1.00 | 18.10 | 8 |
| ATOM | 11502 | N | TYR | C | 1043 | 65.897 | 78.846 | 53.713 | 1.00 | 45.50 | 7 |
| ATOM | 11503 | CA | TYR | C | 1043 | 65.404 | 77.735 | 54.516 | 1.00 | 47.36 | 6 |
| ATOM | 11504 | CB | TYR | C | 1043 | 64.442 | 76.872 | 53.702 | 1.00 | 32.82 | 6 |
| ATOM | 11505 | CG | TYR | C | 1043 | 62.985 | 77.130 | 53.990 | 1.00 | 33.23 | 6 |
| ATOM | 11506 | CD1 | TYR | C | 1043 | 62.043 | 77.093 | 52.977 | 1.00 | 33.36 | 6 |
| ATOM | 11507 | CE1 | TYR | C | 1043 | 60.688 | 77.285 | 53.239 | 1.00 | 35.07 | 6 |
| ATOM | 11508 | CD2 | TYR | C | 1043 | 62.543 | 77.366 | 55.279 | 1.00 | 34.22 | 6 |
| ATOM | 11509 | CE2 | TYR | C | 1043 | 61.191 | 77.556 | 55.555 | 1.00 | 34.94 | 6 |
| ATOM | 11510 | CZ | TYR | C | 1043 | 60.271 | 77.513 | 54.530 | 1.00 | 35.90 | 6 |
| ATOM | 11511 | OH | TYR | C | 1043 | 58.938 | 77.694 | 54.796 | 1.00 | 37.46 | 8 |
| ATOM | 11512 | C | TYR | C | 1043 | 66.609 | 76.918 | 54.949 | 1.00 | 48.24 | 6 |
| ATOM | 11513 | O | TYR | C | 1043 | 67.292 | 77.269 | 55.899 | 1.00 | 50.24 | 8 |
| ATOM | 11514 | N | GLY | C | 1044 | 66.890 | 75.841 | 54.240 | 1.00 | 34.49 | 7 |
| ATOM | 11515 | CA | GLY | C | 1044 | 68.029 | 75.026 | 54.602 | 1.00 | 36.05 | 6 |
| ATOM | 11516 | C | GLY | C | 1044 | 67.912 | 73.702 | 53.899 | 1.00 | 37.06 | 6 |
| ATOM | 11517 | O | GLY | C | 1044 | 68.639 | 72.755 | 54.185 | 1.00 | 38.33 | 8 |
| ATOM | 11518 | N | ALA | C | 1045 | 66.976 | 73.652 | 52.965 | 1.00 | 27.69 | 7 |
| ATOM | 11519 | CA | ALA | C | 1045 | 66.715 | 72.462 | 52.185 | 1.00 | 28.56 | 6 |
| ATOM | 11520 | CB | ALA | C | 1045 | 65.376 | 72.599 | 51.506 | 1.00 | 48.75 | 6 |
| ATOM | 11521 | C | ALA | C | 1045 | 67.802 | 72.241 | 51.146 | 1.00 | 31.05 | 6 |
| ATOM | 11522 | O | ALA | C | 1045 | 67.505 | 71.962 | 49.982 | 1.00 | 31.56 | 8 |
| ATOM | 11523 | N | ALA | C | 1046 | 69.058 | 72.358 | 51.565 | 1.00 | 59.97 | 7 |
| ATOM | 11524 | CA | ALA | C | 1046 | 70.181 | 72.176 | 50.655 | 1.00 | 62.18 | 6 |
| ATOM | 11525 | CB | ALA | C | 1046 | 71.446 | 71.892 | 51.432 | 1.00 | 35.83 | 6 |
| ATOM | 11526 | C | ALA | C | 1046 | 69.911 | 71.053 | 49.664 | 1.00 | 63.92 | 6 |
| ATOM | 11527 | O | ALA | C | 1046 | 69.908 | 71.282 | 48.458 | 1.00 | 65.76 | 8 |
| ATOM | 11528 | N | HIS | C | 1047 | 69.661 | 69.848 | 50.167 | 1.00 | 17.97 | 7 |
| ATOM | 11529 | CA | HIS | C | 1047 | 69.401 | 68.702 | 49.289 | 1.00 | 18.62 | 6 |
| ATOM | 11530 | CB | HIS | C | 1047 | 69.140 | 67.441 | 50.111 | 1.00 | 82.51 | 6 |
| ATOM | 11531 | CG | HIS | C | 1047 | 70.311 | 67.004 | 50.928 | 1.00 | 85.62 | 6 |
| ATOM | 11532 | CD2 | HIS | C | 1047 | 70.498 | 66.961 | 52.267 | 1.00 | 86.23 | 6 |
| ATOM | 11533 | ND1 | HIS | C | 1047 | 71.488 | 66.569 | 50.362 | 1.00 | 87.32 | 7 |
| ATOM | 11534 | CE1 | HIS | C | 1047 | 72.350 | 66.276 | 51.318 | 1.00 | 87.47 | 6 |
| ATOM | 11535 | NE2 | HIS | C | 1047 | 71.773 | 66.505 | 52.484 | 1.00 | 87.30 | 7 |
| ATOM | 11536 | C | HIS | C | 1047 | 68.205 | 68.969 | 48.406 | 1.00 | 18.10 | 6 |
| ATOM | 11537 | O | HIS | C | 1047 | 68.251 | 68.774 | 47.192 | 1.00 | 16.18 | 8 |
| ATOM | 11538 | N | THR | C | 1048 | 67.128 | 69.410 | 49.042 | 1.00 | 41.71 | 7 |
| ATOM | 11539 | CA | THR | C | 1048 | 65.890 | 69.716 | 48.352 | 1.00 | 42.95 | 6 |
| ATOM | 11540 | CB | THR | C | 1048 | 64.840 | 70.267 | 49.349 | 1.00 | 25.95 | 6 |
| ATOM | 11541 | OG1 | THR | C | 1048 | 64.529 | 69.265 | 50.326 | 1.00 | 24.53 | 8 |
| ATOM | 11542 | CG2 | THR | C | 1048 | 63.574 | 70.664 | 48.636 | 1.00 | 25.54 | 6 |
| ATOM | 11543 | C | THR | C | 1048 | 66.194 | 70.736 | 47.258 | 1.00 | 44.10 | 6 |
| ATOM | 11544 | O | THR | C | 1048 | 65.448 | 70.859 | 46.285 | 1.00 | 45.27 | 8 |
| ATOM | 11545 | N | LEU | C | 1049 | 67.300 | 71.457 | 47.414 | 1.00 | 20.73 | 7 |
| ATOM | 11546 | CA | LEU | C | 1049 | 67.689 | 72.436 | 46.416 | 1.00 | 22.41 | 6 |
| ATOM | 11547 | CB | LEU | C | 1049 | 68.462 | 73.580 | 47.046 | 1.00 | 14.67 | 6 |
| ATOM | 11548 | CG | LEU | C | 1049 | 69.024 | 74.548 | 46.008 | 1.00 | 13.87 | 6 |
| ATOM | 11549 | CD1 | LEU | C | 1049 | 67.920 | 75.428 | 45.470 | 1.00 | 13.87 | 6 |
| ATOM | 11550 | CD2 | LEU | C | 1049 | 70.105 | 75.393 | 46.635 | 1.00 | 13.87 | 6 |
| ATOM | 11551 | C | LEU | C | 1049 | 68.532 | 71.800 | 45.307 | 1.00 | 25.25 | 6 |
| ATOM | 11552 | O | LEU | C | 1049 | 68.120 | 71.802 | 44.153 | 1.00 | 26.31 | 8 |
| ATOM | 11553 | N | GLN | C | 1050 | 69.699 | 71.253 | 45.643 | 1.00 | 68.14 | 7 |
| ATOM | 11554 | CA | GLN | C | 1050 | 70.576 | 70.610 | 44.646 | 1.00 | 70.78 | 6 |
| ATOM | 11555 | CB | GLN | C | 1050 | 71.694 | 69.829 | 45.351 | 1.00 | 70.95 | 6 |
| ATOM | 11556 | CG | GLN | C | 1050 | 72.576 | 68.969 | 44.445 | 1.00 | 71.54 | 6 |
| ATOM | 11557 | CD | GLN | C | 1050 | 73.733 | 68.324 | 45.201 | 1.00 | 73.07 | 6 |
| ATOM | 11558 | OE1 | GLN | C | 1050 | 73.534 | 67.546 | 46.138 | 1.00 | 73.69 | 8 |
| ATOM | 11559 | NE2 | GLN | C | 1050 | 74.951 | 68.652 | 44.795 | 1.00 | 73.59 | 7 |
| ATOM | 11560 | C | GLN | C | 1050 | 69.808 | 69.660 | 43.728 | 1.00 | 72.51 | 6 |
| ATOM | 11561 | O | GLN | C | 1050 | 70.227 | 69.362 | 42.612 | 1.00 | 74.13 | 8 |
| ATOM | 11562 | N | GLU | C | 1051 | 68.677 | 69.190 | 44.227 | 1.00 | 47.00 | 7 |
| ATOM | 11563 | CA | GLU | C | 1051 | 67.801 | 68.269 | 43.517 | 1.00 | 47.80 | 6 |
| ATOM | 11564 | CB | GLU | C | 1051 | 66.749 | 67.772 | 44.516 | 1.00 | 87.07 | 6 |
| ATOM | 11565 | CG | GLU | C | 1051 | 65.719 | 66.820 | 43.979 | 1.00 | 89.43 | 6 |
| ATOM | 11566 | CD | GLU | C | 1051 | 64.757 | 66.371 | 45.059 | 1.00 | 91.00 | 6 |
| ATOM | 11567 | OE1 | GLU | C | 1051 | 64.244 | 67.239 | 45.798 | 1.00 | 91.45 | 8 |
| ATOM | 11568 | OE2 | GLU | C | 1051 | 64.508 | 65.154 | 45.163 | 1.00 | 91.93 | 8 |
| ATOM | 11569 | C | GLU | C | 1051 | 67.142 | 68.955 | 42.320 | 1.00 | 46.49 | 6 |
| ATOM | 11570 | O | GLU | C | 1051 | 66.878 | 68.343 | 41.283 | 1.00 | 46.46 | 8 |
| ATOM | 11571 | N | MET | C | 1052 | 66.894 | 70.244 | 42.479 | 1.00 | 33.53 | 7 |
| ATOM | 11572 | CA | MET | C | 1052 | 66.252 | 71.033 | 41.452 | 1.00 | 32.50 | 6 |
| ATOM | 11573 | CB | MET | C | 1052 | 65.346 | 72.086 | 42.112 | 1.00 | 52.50 | 6 |
| ATOM | 11574 | CG | MET | C | 1052 | 63.843 | 71.788 | 41.962 | 1.00 | 54.28 | 6 |
| ATOM | 11575 | SD | MET | C | 1052 | 62.727 | 72.440 | 43.248 | 1.00 | 55.26 | 16 |
| ATOM | 11576 | CE | MET | C | 1052 | 61.470 | 71.163 | 43.275 | 1.00 | 53.55 | 6 |
| ATOM | 11577 | C | MET | C | 1052 | 67.248 | 71.682 | 40.501 | 1.00 | 30.92 | 6 |
| ATOM | 11578 | O | MET | C | 1052 | 66.957 | 72.690 | 39.876 | 1.00 | 30.06 | 8 |
| ATOM | 11579 | N | LEU | C | 1053 | 68.432 | 71.105 | 40.395 | 1.00 | 68.89 | 7 |
| ATOM | 11580 | CA | LEU | C | 1053 | 69.443 | 71.627 | 39.490 | 1.00 | 68.99 | 6 |
| ATOM | 11581 | CB | LEU | C | 1053 | 70.306 | 72.684 | 40.179 | 1.00 | 67.82 | 6 |
| ATOM | 11582 | CG | LEU | C | 1053 | 69.706 | 73.932 | 40.839 | 1.00 | 69.43 | 6 |
| ATOM | 11583 | CD1 | LEU | C | 1053 | 68.706 | 74.629 | 39.927 | 1.00 | 68.76 | 6 |
| ATOM | 11584 | CD2 | LEU | C | 1053 | 69.050 | 73.528 | 42.126 | 1.00 | 70.12 | 6 |
| ATOM | 11585 | C | LEU | C | 1053 | 70.295 | 70.423 | 39.138 | 1.00 | 68.46 | 6 |
| ATOM | 11586 | O | LEU | C | 1053 | 71.409 | 70.545 | 38.637 | 1.00 | 69.90 | 8 |
| ATOM | 11587 | N | THR | C | 1054 | 69.736 | 69.250 | 39.405 | 1.00 | 39.11 | 7 |
| ATOM | 11588 | CA | THR | C | 1054 | 70.414 | 67.983 | 39.177 | 1.00 | 35.96 | 6 |
| ATOM | 11589 | CB | THR | C | 1054 | 71.110 | 67.521 | 40.479 | 1.00 | 59.10 | 6 |
| ATOM | 11590 | OG1 | THR | C | 1054 | 71.969 | 68.562 | 40.951 | 1.00 | 60.89 | 8 |
| ATOM | 11591 | CG2 | THR | C | 1054 | 71.927 | 66.274 | 40.244 | 1.00 | 60.72 | 6 |
| ATOM | 11592 | C | THR | C | 1054 | 69.382 | 66.932 | 38.774 | 1.00 | 33.47 | 6 |
| ATOM | 11593 | O | THR | C | 1054 | 68.821 | 66.966 | 37.677 | 1.00 | 32.96 | 8 |
| ATOM | 11594 | N | ILE | C | 1055 | 69.147 | 66.007 | 39.696 | 1.00 | 24.25 | 7 |
| ATOM | 11595 | CA | ILE | C | 1055 | 68.202 | 64.917 | 39.547 | 1.00 | 22.76 | 6 |
| ATOM | 11596 | CB | ILE | C | 1055 | 67.863 | 64.323 | 40.945 | 1.00 | 71.16 | 6 |
| ATOM | 11597 | CG2 | ILE | C | 1055 | 67.064 | 63.050 | 40.814 | 1.00 | 73.15 | 6 |
| ATOM | 11598 | CG1 | ILE | C | 1055 | 69.152 | 64.015 | 41.708 | 1.00 | 73.03 | 6 |
| ATOM | 11599 | CD1 | ILE | C | 1055 | 69.823 | 65.230 | 42.326 | 1.00 | 74.82 | 6 |
| ATOM | 11600 | C | ILE | C | 1055 | 66.901 | 65.350 | 38.861 | 1.00 | 20.58 | 6 |
| ATOM | 11601 | O | ILE | C | 1055 | 66.015 | 64.530 | 38.636 | 1.00 | 19.60 | 8 |
| ATOM | 11602 | N | LYS | C | 1056 | 66.759 | 66.620 | 38.509 | 1.00 | 30.64 | 7 |
| ATOM | 11603 | CA | LYS | C | 1056 | 65.512 | 67.010 | 37.867 | 1.00 | 29.46 | 6 |
| ATOM | 11604 | CB | LYS | C | 1056 | 64.611 | 67.710 | 38.898 | 1.00 | 37.57 | 6 |
| ATOM | 11605 | CG | LYS | C | 1056 | 64.300 | 66.874 | 40.146 | 1.00 | 36.56 | 6 |
| ATOM | 11606 | CD | LYS | C | 1056 | 62.971 | 67.288 | 40.790 | 1.00 | 36.77 | 6 |
| ATOM | 11607 | CE | LYS | C | 1056 | 63.164 | 67.898 | 42.175 | 1.00 | 36.68 | 6 |
| ATOM | 11608 | NZ | LYS | C | 1056 | 61.899 | 68.327 | 42.849 | 1.00 | 36.50 | 7 |
| ATOM | 11609 | C | LYS | C | 1056 | 65.588 | 67.850 | 36.581 | 1.00 | 28.70 | 6 |
| ATOM | 11610 | O | LYS | C | 1056 | 64.594 | 68.448 | 36.175 | 1.00 | 27.32 | 8 |
| ATOM | 11611 | N | SER | C | 1057 | 66.728 | 67.889 | 35.907 | 1.00 | 33.53 | 7 |
| ATOM | 11612 | CA | SER | C | 1057 | 66.763 | 68.714 | 34.711 | 1.00 | 34.79 | 6 |
| ATOM | 11613 | CB | SER | C | 1057 | 66.781 | 70.191 | 35.143 | 1.00 | 41.35 | 6 |
| ATOM | 11614 | OG | SER | C | 1057 | 66.640 | 71.087 | 34.055 | 1.00 | 40.46 | 8 |
| ATOM | 11615 | C | SER | C | 1057 | 67.892 | 68.432 | 33.709 | 1.00 | 36.44 | 6 |
| ATOM | 11616 | O | SER | C | 1057 | 67.652 | 67.918 | 32.609 | 1.00 | 36.41 | 8 |
| ATOM | 11617 | N | ASP | C | 1058 | 69.123 | 68.756 | 34.086 | 1.00 | 41.70 | 7 |
| ATOM | 11618 | CA | ASP | C | 1058 | 70.250 | 68.572 | 33.189 | 1.00 | 42.89 | 6 |
| ATOM | 11619 | CB | ASP | C | 1058 | 71.175 | 69.768 | 33.307 | 1.00 | 72.96 | 6 |
| ATOM | 11620 | CG | ASP | C | 1058 | 70.419 | 71.041 | 33.543 | 1.00 | 75.70 | 6 |
| ATOM | 11621 | OD1 | ASP | C | 1058 | 69.565 | 71.384 | 32.697 | 1.00 | 76.87 | 8 |
| ATOM | 11622 | OD2 | ASP | C | 1058 | 70.670 | 71.691 | 34.580 | 1.00 | 76.41 | 8 |
| ATOM | 11623 | C | ASP | C | 1058 | 71.037 | 67.315 | 33.453 | 1.00 | 43.10 | 6 |
| ATOM | 11624 | O | ASP | C | 1058 | 71.460 | 66.637 | 32.514 | 1.00 | 44.09 | 8 |
| ATOM | 11625 | N | ASP | C | 1059 | 71.240 | 67.022 | 34.734 | 1.00 | 40.94 | 7 |
| ATOM | 11626 | CA | ASP | C | 1059 | 72.009 | 65.860 | 35.169 | 1.00 | 40.89 | 6 |
| ATOM | 11627 | CB | ASP | C | 1059 | 72.100 | 65.851 | 36.693 | 1.00 | 59.93 | 6 |
| ATOM | 11628 | CG | ASP | C | 1059 | 73.229 | 64.985 | 37.209 | 1.00 | 59.94 | 6 |
| ATOM | 11629 | OD1 | ASP | C | 1059 | 73.206 | 63.754 | 36.993 | 1.00 | 60.63 | 8 |
| ATOM | 11630 | OD2 | ASP | C | 1059 | 74.145 | 65.550 | 37.840 | 1.00 | 59.46 | 8 |
| ATOM | 11631 | C | ASP | C | 1059 | 71.392 | 64.564 | 34.677 | 1.00 | 41.14 | 6 |
| ATOM | 11632 | O | ASP | C | 1059 | 70.647 | 63.904 | 35.404 | 1.00 | 41.24 | 8 |
| ATOM | 11633 | N | ILE | C | 1060 | 71.723 | 64.200 | 33.439 | 1.00 | 29.51 | 7 |
| ATOM | 11634 | CA | ILE | C | 1060 | 71.194 | 62.996 | 32.809 | 1.00 | 29.42 | 6 |
| ATOM | 11635 | CB | ILE | C | 1060 | 71.833 | 62.799 | 31.440 | 1.00 | 33.65 | 6 |
| ATOM | 11636 | CG2 | ILE | C | 1060 | 71.690 | 61.376 | 30.984 | 1.00 | 33.67 | 6 |
| ATOM | 11637 | CG1 | ILE | C | 1060 | 71.161 | 63.768 | 30.469 | 1.00 | 35.61 | 6 |
| ATOM | 11638 | CD1 | ILE | C | 1060 | 71.539 | 63.619 | 29.014 | 1.00 | 39.90 | 6 |
| ATOM | 11639 | C | ILE | C | 1060 | 71.348 | 61.755 | 33.667 | 1.00 | 29.48 | 6 |
| ATOM | 11640 | O | ILE | C | 1060 | 70.389 | 60.999 | 33.844 | 1.00 | 28.33 | 8 |
| ATOM | 11641 | N | GLU | C | 1061 | 72.554 | 61.552 | 34.192 | 1.00 | 42.18 | 7 |
| ATOM | 11642 | CA | GLU | C | 1061 | 72.825 | 60.425 | 35.070 | 1.00 | 42.34 | 6 |
| ATOM | 11643 | CB | GLU | C | 1061 | 74.230 | 60.533 | 35.672 | 1.00 | 93.65 | 6 |
| ATOM | 11644 | CG | GLU | C | 1061 | 75.328 | 59.915 | 34.818 | 1.00 | 98.94 | 6 |
| ATOM | 11645 | CD | GLU | C | 1061 | 75.348 | 58.392 | 34.889 | 1.00 | 101.92 | 6 |
| ATOM | 11646 | OE1 | GLU | C | 1061 | 74.277 | 57.771 | 34.706 | 1.00 | 102.54 | 8 |
| ATOM | 11647 | OE2 | GLU | C | 1061 | 76.437 | 57.815 | 35.118 | 1.00 | 104.01 | 8 |
| ATOM | 11648 | C | GLU | C | 1061 | 71.792 | 60.529 | 36.166 | 1.00 | 41.28 | 6 |
| ATOM | 11649 | O | GLU | C | 1061 | 70.786 | 59.832 | 36.139 | 1.00 | 41.16 | 8 |
| ATOM | 11650 | N | GLY | C | 1062 | 72.043 | 61.430 | 37.109 | 1.00 | 39.43 | 7 |
| ATOM | 11651 | CA | GLY | C | 1062 | 71.135 | 61.637 | 38.223 | 1.00 | 39.42 | 6 |
| ATOM | 11652 | C | GLY | C | 1062 | 69.658 | 61.633 | 37.873 | 1.00 | 39.41 | 6 |
| ATOM | 11653 | O | GLY | C | 1062 | 68.844 | 61.044 | 38.593 | 1.00 | 38.93 | 8 |
| ATOM | 11654 | N | ALA | C | 1063 | 69.299 | 62.298 | 36.781 | 1.00 | 30.84 | 7 |
| ATOM | 11655 | CA | ALA | C | 1063 | 67.903 | 62.332 | 36.359 | 1.00 | 30.59 | 6 |
| ATOM | 11656 | CB | ALA | C | 1063 | 67.787 | 62.893 | 34.957 | 1.00 | 25.00 | 6 |
| ATOM | 11657 | C | ALA | C | 1063 | 67.402 | 60.909 | 36.379 | 1.00 | 31.03 | 6 |
| ATOM | 11658 | O | ALA | C | 1063 | 66.524 | 60.565 | 37.159 | 1.00 | 30.00 | 8 |
| ATOM | 11659 | N | ASN | C | 1064 | 68.006 | 60.089 | 35.522 | 1.00 | 47.03 | 7 |
| ATOM | 11660 | CA | ASN | C | 1064 | 67.670 | 58.676 | 35.380 | 1.00 | 47.79 | 6 |
| ATOM | 11661 | CB | ASN | C | 1064 | 68.636 | 58.011 | 34.409 | 1.00 | 61.15 | 6 |
| ATOM | 11662 | CG | ASN | C | 1064 | 68.922 | 58.873 | 33.197 | 1.00 | 62.05 | 6 |
| ATOM | 11663 | OD1 | ASN | C | 1064 | 68.104 | 59.695 | 32.793 | 1.00 | 61.27 | 8 |
| ATOM | 11664 | ND2 | ASN | C | 1064 | 70.089 | 58.678 | 32.602 | 1.00 | 65.22 | 7 |
| ATOM | 11665 | C | ASN | C | 1064 | 67.684 | 57.930 | 36.707 | 1.00 | 48.16 | 6 |
| ATOM | 11666 | O | ASN | C | 1064 | 66.871 | 57.033 | 36.932 | 1.00 | 47.80 | 8 |
| ATOM | 11667 | N | ALA | C | 1065 | 68.614 | 58.293 | 37.582 | 1.00 | 40.42 | 7 |
| ATOM | 11668 | CA | ALA | C | 1065 | 68.692 | 57.667 | 38.894 | 1.00 | 42.32 | 6 |
| ATOM | 11669 | CB | ALA | C | 1065 | 69.823 | 58.272 | 39.703 | 1.00 | 116.17 | 6 |
| ATOM | 11670 | C | ALA | C | 1065 | 67.366 | 57.943 | 39.570 | 1.00 | 43.55 | 6 |
| ATOM | 11671 | O | ALA | C | 1065 | 66.959 | 57.215 | 40.457 | 1.00 | 43.00 | 8 |
| ATOM | 11672 | N | ALA | C | 1066 | 66.693 | 59.003 | 39.134 | 1.00 | 53.27 | 7 |
| ATOM | 11673 | CA | ALA | C | 1066 | 65.398 | 59.367 | 39.695 | 1.00 | 55.91 | 6 |
| ATOM | 11674 | CB | ALA | C | 1066 | 65.098 | 60.833 | 39.421 | 1.00 | 50.08 | 6 |
| ATOM | 11675 | C | ALA | C | 1066 | 64.279 | 58.496 | 39.134 | 1.00 | 57.12 | 6 |
| ATOM | 11676 | O | ALA | C | 1066 | 63.570 | 57.829 | 39.880 | 1.00 | 57.12 | 8 |
| ATOM | 11677 | N | TYR | C | 1067 | 64.116 | 58.505 | 37.818 | 1.00 | 66.31 | 7 |
| ATOM | 11678 | CA | TYR | C | 1067 | 63.069 | 57.710 | 37.207 | 1.00 | 67.30 | 6 |
| ATOM | 11679 | CB | TYR | C | 1067 | 62.896 | 58.062 | 35.732 | 1.00 | 78.92 | 6 |
| ATOM | 11680 | CG | TYR | C | 1067 | 61.901 | 57.150 | 35.083 | 1.00 | 79.83 | 6 |
| ATOM | 11681 | CD1 | TYR | C | 1067 | 60.603 | 57.066 | 35.571 | 1.00 | 80.62 | 6 |
| ATOM | 11682 | CE1 | TYR | C | 1067 | 59.710 | 56.122 | 35.083 | 1.00 | 81.65 | 6 |
| ATOM | 11683 | CD2 | TYR | C | 1067 | 62.282 | 56.275 | 34.076 | 1.00 | 79.94 | 6 |
| ATOM | 11684 | CE2 | TYR | C | 1067 | 61.397 | 55.325 | 33.578 | 1.00 | 80.93 | 6 |
| ATOM | 11685 | CZ | TYR | C | 1067 | 60.113 | 55.252 | 34.092 | 1.00 | 81.31 | 6 |
| ATOM | 11686 | OH | TYR | C | 1067 | 59.242 | 54.286 | 33.643 | 1.00 | 81.64 | 8 |
| ATOM | 11687 | C | TYR | C | 1067 | 63.332 | 56.218 | 37.337 | 1.00 | 68.80 | 6 |
| ATOM | 11688 | O | TYR | C | 1067 | 62.598 | 55.402 | 36.781 | 1.00 | 67.61 | 8 |
| ATOM | 11689 | N | GLN | C | 1068 | 64.379 | 55.851 | 38.065 | 1.00 | 43.61 | 7 |
| ATOM | 11690 | CA | GLN | C | 1068 | 64.671 | 54.435 | 38.240 | 1.00 | 46.88 | 6 |
| ATOM | 11691 | CB | GLN | C | 1068 | 65.991 | 54.064 | 37.565 | 1.00 | 108.79 | 6 |
| ATOM | 11692 | CG | GLN | C | 1068 | 66.158 | 52.564 | 37.355 | 1.00 | 110.39 | 6 |
| ATOM | 11693 | CD | GLN | C | 1068 | 67.334 | 52.221 | 36.458 | 1.00 | 112.30 | 6 |
| ATOM | 11694 | OE1 | GLN | C | 1068 | 68.490 | 52.474 | 36.806 | 1.00 | 113.10 | 8 |
| ATOM | 11695 | NE2 | GLN | C | 1068 | 67.044 | 51.648 | 35.292 | 1.00 | 112.89 | 7 |
| ATOM | 11696 | C | GLN | C | 1068 | 64.710 | 54.046 | 39.708 | 1.00 | 48.56 | 6 |
| ATOM | 11697 | O | GLN | C | 1068 | 64.021 | 53.124 | 40.126 | 1.00 | 48.70 | 8 |
| ATOM | 11698 | N | ALA | C | 1069 | 65.508 | 54.750 | 40.498 | 1.00 | 65.14 | 7 |
| ATOM | 11699 | CA | ALA | C | 1069 | 65.600 | 54.444 | 41.918 | 1.00 | 67.74 | 6 |
| ATOM | 11700 | CB | ALA | C | 1069 | 66.426 | 55.497 | 42.624 | 1.00 | 132.45 | 6 |
| ATOM | 11701 | C | ALA | C | 1069 | 64.220 | 54.356 | 42.562 | 1.00 | 69.46 | 6 |
| ATOM | 11702 | O | ALA | C | 1069 | 64.072 | 53.772 | 43.640 | 1.00 | 70.12 | 8 |
| ATOM | 11703 | N | ILE | C | 1070 | 63.219 | 54.944 | 41.901 | 1.00 | 60.17 | 7 |
| ATOM | 11704 | CA | ILE | C | 1070 | 61.837 | 54.940 | 42.391 | 1.00 | 61.46 | 6 |
| ATOM | 11705 | CB | ILE | C | 1070 | 61.097 | 56.264 | 42.055 | 1.00 | 86.63 | 6 |
| ATOM | 11706 | CG2 | ILE | C | 1070 | 59.612 | 56.117 | 42.328 | 1.00 | 86.27 | 6 |
| ATOM | 11707 | CG1 | ILE | C | 1070 | 61.634 | 57.413 | 42.915 | 1.00 | 86.82 | 6 |
| ATOM | 11708 | CD1 | ILE | C | 1070 | 63.080 | 57.786 | 42.663 | 1.00 | 87.50 | 6 |
| ATOM | 11709 | C | ILE | C | 1070 | 61.101 | 53.788 | 41.734 | 1.00 | 62.66 | 6 |
| ATOM | 11710 | O | ILE | C | 1070 | 60.550 | 52.927 | 42.414 | 1.00 | 62.18 | 8 |
| ATOM | 11711 | N | ILE | C | 1071 | 61.104 | 53.779 | 40.405 | 1.00 | 97.42 | 7 |
| ATOM | 11712 | CA | ILE | C | 1071 | 60.455 | 52.722 | 39.645 | 1.00 | 100.40 | 6 |
| ATOM | 11713 | CB | ILE | C | 1071 | 60.830 | 52.798 | 38.144 | 1.00 | 191.02 | 6 |
| ATOM | 11714 | CG2 | ILE | C | 1071 | 60.548 | 51.477 | 37.457 | 1.00 | 190.29 | 6 |
| ATOM | 11715 | CG1 | ILE | C | 1071 | 60.015 | 53.897 | 37.464 | 1.00 | 192.69 | 6 |
| ATOM | 11716 | CD1 | ILE | C | 1071 | 60.073 | 55.238 | 38.168 | 1.00 | 195.25 | 6 |
| ATOM | 11717 | C | ILE | C | 1071 | 60.932 | 51.415 | 40.241 | 1.00 | 101.61 | 6 |
| ATOM | 11718 | O | ILE | C | 1071 | 60.220 | 50.412 | 40.227 | 1.00 | 102.61 | 8 |
| ATOM | 11719 | N | LYS | C | 1072 | 62.147 | 51.443 | 40.779 | 1.00 | 94.80 | 7 |
| ATOM | 11720 | CA | LYS | C | 1072 | 62.712 | 50.269 | 41.410 | 1.00 | 96.43 | 6 |
| ATOM | 11721 | CB | LYS | C | 1072 | 64.188 | 50.479 | 41.747 | 1.00 | 112.40 | 6 |
| ATOM | 11722 | CG | LYS | C | 1072 | 65.154 | 50.269 | 40.588 | 1.00 | 113.75 | 6 |
| ATOM | 11723 | CD | LYS | C | 1072 | 66.592 | 50.345 | 41.091 | 1.00 | 113.38 | 6 |
| ATOM | 11724 | CE | LYS | C | 1072 | 67.602 | 50.149 | 39.977 | 1.00 | 113.27 | 6 |
| ATOM | 11725 | NZ | LYS | C | 1072 | 68.993 | 50.337 | 40.481 | 1.00 | 112.40 | 7 |
| ATOM | 11726 | C | LYS | C | 1072 | 61.933 | 50.021 | 42.686 | 1.00 | 97.49 | 6 |
| ATOM | 11727 | O | LYS | C | 1072 | 60.939 | 49.295 | 42.678 | 1.00 | 98.64 | 8 |
| ATOM | 11728 | N | GLY | C | 1073 | 62.368 | 50.631 | 43.782 | 1.00 | 108.77 | 7 |
| ATOM | 11729 | CA | GLY | C | 1073 | 61.663 | 50.415 | 45.029 | 1.00 | 110.58 | 6 |
| ATOM | 11730 | C | GLY | C | 1073 | 62.126 | 51.185 | 46.246 | 1.00 | 111.42 | 6 |
| ATOM | 11731 | O | GLY | C | 1073 | 62.131 | 52.413 | 46.261 | 1.00 | 111.19 | 8 |
| ATOM | 11732 | N | ALA | C | 1074 | 62.521 | 50.443 | 47.272 | 1.00 | 197.22 | 7 |
| ATOM | 11733 | CA | ALA | C | 1074 | 62.950 | 51.018 | 48.538 | 1.00 | 199.58 | 6 |
| ATOM | 11734 | CB | ALA | C | 1074 | 63.233 | 49.893 | 49.535 | 1.00 | 161.27 | 6 |
| ATOM | 11735 | C | ALA | C | 1074 | 64.122 | 51.999 | 48.536 | 1.00 | 200.96 | 6 |
| ATOM | 11736 | O | ALA | C | 1074 | 64.683 | 52.260 | 49.599 | 1.00 | 201.96 | 8 |
| ATOM | 11737 | N | ASP | C | 1075 | 64.504 | 52.558 | 47.388 | 1.00 | 106.63 | 7 |
| ATOM | 11738 | CA | ASP | C | 1075 | 65.621 | 53.504 | 47.413 | 1.00 | 106.82 | 6 |
| ATOM | 11739 | CB | ASP | C | 1075 | 66.910 | 52.832 | 46.930 | 1.00 | 182.53 | 6 |
| ATOM | 11740 | CG | ASP | C | 1075 | 68.151 | 53.417 | 47.594 | 1.00 | 183.78 | 6 |
| ATOM | 11741 | OD1 | ASP | C | 1075 | 68.332 | 54.653 | 47.540 | 1.00 | 184.11 | 8 |
| ATOM | 11742 | OD2 | ASP | C | 1075 | 68.944 | 52.642 | 48.173 | 1.00 | 184.82 | 8 |
| ATOM | 11743 | C | ASP | C | 1075 | 65.397 | 54.805 | 46.649 | 1.00 | 105.85 | 6 |
| ATOM | 11744 | O | ASP | C | 1075 | 64.748 | 54.828 | 45.607 | 1.00 | 104.76 | 8 |
| ATOM | 11745 | N | VAL | C | 1076 | 65.954 | 55.888 | 47.186 | 1.00 | 64.03 | 7 |
| ATOM | 11746 | CA | VAL | C | 1076 | 65.817 | 57.213 | 46.593 | 1.00 | 65.28 | 6 |
| ATOM | 11747 | CB | VAL | C | 1076 | 65.431 | 58.225 | 47.671 | 1.00 | 191.41 | 6 |
| ATOM | 11748 | CG1 | VAL | C | 1076 | 63.923 | 58.256 | 47.825 | 1.00 | 192.81 | 6 |
| ATOM | 11749 | CG2 | VAL | C | 1076 | 66.087 | 57.840 | 48.994 | 1.00 | 192.40 | 6 |
| ATOM | 11750 | C | VAL | C | 1076 | 67.070 | 57.714 | 45.872 | 1.00 | 65.50 | 6 |
| ATOM | 11751 | O | VAL | C | 1076 | 68.178 | 57.284 | 46.176 | 1.00 | 65.51 | 8 |
| ATOM | 11752 | N | PRO | C | 1077 | 66.902 | 58.647 | 44.917 | 1.00 | 111.04 | 7 |
| ATOM | 11753 | CD | PRO | C | 1077 | 65.573 | 59.247 | 44.689 | 1.00 | 70.63 | 6 |
| ATOM | 11754 | CA | PRO | C | 1077 | 67.913 | 59.298 | 44.070 | 1.00 | 111.78 | 6 |
| ATOM | 11755 | CB | PRO | C | 1077 | 67.199 | 60.568 | 43.629 | 1.00 | 70.35 | 6 |
| ATOM | 11756 | CG | PRO | C | 1077 | 65.788 | 60.092 | 43.455 | 1.00 | 70.28 | 6 |
| ATOM | 11757 | C | PRO | C | 1077 | 69.266 | 59.600 | 44.720 | 1.00 | 112.57 | 6 |
| ATOM | 11758 | O | PRO | C | 1077 | 69.401 | 60.583 | 45.438 | 1.00 | 113.94 | 8 |
| ATOM | 11759 | N | GLU | C | 1078 | 70.271 | 58.774 | 44.447 | 1.00 | 187.01 | 7 |
| ATOM | 11760 | CA | GLU | C | 1078 | 71.597 | 58.981 | 45.029 | 1.00 | 187.30 | 6 |
| ATOM | 11761 | CB | GLU | C | 1078 | 72.207 | 57.636 | 45.458 | 1.00 | 208.87 | 6 |
| ATOM | 11762 | CG | GLU | C | 1078 | 71.522 | 56.942 | 46.631 | 1.00 | 208.87 | 6 |
| ATOM | 11763 | CD | GLU | C | 1078 | 72.300 | 55.732 | 47.123 | 1.00 | 208.87 | 6 |
| ATOM | 11764 | OE1 | GLU | C | 1078 | 73.466 | 55.905 | 47.538 | 1.00 | 208.87 | 8 |
| ATOM | 11765 | OE2 | GLU | C | 1078 | 71.751 | 54.609 | 47.090 | 1.00 | 208.87 | 8 |
| ATOM | 11766 | C | GLU | C | 1078 | 72.582 | 59.693 | 44.093 | 1.00 | 186.18 | 6 |
| ATOM | 11767 | O | GLU | C | 1078 | 73.208 | 59.052 | 43.249 | 1.00 | 187.10 | 8 |
| ATOM | 11768 | N | PRO | C | 1079 | 72.731 | 61.026 | 44.225 | 1.00 | 130.53 | 7 |
| ATOM | 11769 | CD | PRO | C | 1079 | 71.867 | 61.981 | 44.936 | 1.00 | 103.91 | 6 |
| ATOM | 11770 | CA | PRO | C | 1079 | 73.673 | 61.737 | 43.347 | 1.00 | 129.47 | 6 |
| ATOM | 11771 | CB | PRO | C | 1079 | 73.234 | 63.197 | 43.451 | 1.00 | 103.50 | 6 |
| ATOM | 11772 | CG | PRO | C | 1079 | 71.819 | 63.118 | 43.956 | 1.00 | 103.40 | 6 |
| ATOM | 11773 | C | PRO | C | 1079 | 75.096 | 61.557 | 43.851 | 1.00 | 128.55 | 6 |
| ATOM | 11774 | O | PRO | C | 1079 | 75.384 | 60.626 | 44.603 | 1.00 | 128.85 | 8 |
| ATOM | 11775 | N | SER | C | 1080 | 75.977 | 62.463 | 43.442 | 1.00 | 111.85 | 7 |
| ATOM | 11776 | CA | SER | C | 1080 | 77.379 | 62.436 | 43.853 | 1.00 | 110.74 | 6 |
| ATOM | 11777 | CB | SER | C | 1080 | 78.015 | 61.065 | 43.547 | 1.00 | 111.30 | 6 |
| ATOM | 11778 | OG | SER | C | 1080 | 77.944 | 60.727 | 42.169 | 1.00 | 111.57 | 8 |
| ATOM | 11779 | C | SER | C | 1080 | 78.114 | 63.548 | 43.113 | 1.00 | 109.52 | 6 |
| ATOM | 11780 | O | SER | C | 1080 | 78.454 | 64.585 | 43.688 | 1.00 | 109.21 | 8 |
| ATOM | 11781 | N | VAL | C | 1081 | 78.352 | 63.325 | 41.828 | 1.00 | 103.22 | 7 |
| ATOM | 11782 | CA | VAL | C | 1081 | 79.011 | 64.310 | 40.986 | 1.00 | 101.81 | 6 |
| ATOM | 11783 | CB | VAL | C | 1081 | 79.813 | 63.628 | 39.853 | 1.00 | 208.87 | 6 |
| ATOM | 11784 | CG1 | VAL | C | 1081 | 80.636 | 64.657 | 39.101 | 1.00 | 208.87 | 6 |
| ATOM | 11785 | CG2 | VAL | C | 1081 | 80.704 | 62.540 | 40.424 | 1.00 | 208.87 | 6 |
| ATOM | 11786 | C | VAL | C | 1081 | 77.879 | 65.129 | 40.364 | 1.00 | 99.68 | 6 |
| ATOM | 11787 | O | VAL | C | 1081 | 76.917 | 64.577 | 39.835 | 1.00 | 99.10 | 8 |
| ATOM | 11788 | N | PRO | C | 1082 | 77.951 | 66.456 | 40.458 | 1.00 | 54.33 | 7 |
| ATOM | 11789 | CD | PRO | C | 1082 | 78.776 | 67.272 | 41.363 | 1.00 | 200.29 | 6 |
| ATOM | 11790 | CA | PRO | C | 1082 | 76.878 | 67.244 | 39.861 | 1.00 | 52.51 | 6 |
| ATOM | 11791 | CB | PRO | C | 1082 | 76.885 | 68.513 | 40.699 | 1.00 | 198.94 | 6 |
| ATOM | 11792 | CG | PRO | C | 1082 | 78.331 | 68.671 | 41.026 | 1.00 | 200.17 | 6 |
| ATOM | 11793 | C | PRO | C | 1082 | 77.158 | 67.503 | 38.382 | 1.00 | 51.25 | 6 |
| ATOM | 11794 | O | PRO | C | 1082 | 78.189 | 68.066 | 38.014 | 1.00 | 51.33 | 8 |
| ATOM | 11795 | N | GLU | C | 1083 | 76.230 | 67.072 | 37.540 | 1.00 | 56.19 | 7 |
| ATOM | 11796 | CA | GLU | C | 1083 | 76.328 | 67.242 | 36.097 | 1.00 | 55.63 | 6 |
| ATOM | 11797 | CB | GLU | C | 1083 | 74.957 | 66.993 | 35.465 | 1.00 | 190.05 | 6 |
| ATOM | 11798 | CG | GLU | C | 1083 | 74.875 | 67.163 | 33.954 | 1.00 | 192.19 | 6 |
| ATOM | 11799 | CD | GLU | C | 1083 | 74.970 | 65.847 | 33.206 | 1.00 | 193.81 | 6 |
| ATOM | 11800 | OE1 | GLU | C | 1083 | 74.245 | 64.896 | 33.570 | 1.00 | 194.43 | 8 |
| ATOM | 11801 | OE2 | GLU | C | 1083 | 75.762 | 65.764 | 32.244 | 1.00 | 194.82 | 8 |
| ATOM | 11802 | C | GLU | C | 1083 | 76.809 | 68.639 | 35.719 | 1.00 | 53.58 | 6 |
| ATOM | 11803 | O | GLU | C | 1083 | 77.360 | 68.841 | 34.643 | 1.00 | 53.37 | 8 |
| ATOM | 11804 | N | SER | C | 1084 | 76.588 | 69.605 | 36.599 | 1.00 | 91.83 | 7 |
| ATOM | 11805 | CA | SER | C | 1084 | 77.005 | 70.963 | 36.313 | 1.00 | 91.20 | 6 |
| ATOM | 11806 | CB | SER | C | 1084 | 76.183 | 71.953 | 37.129 | 1.00 | 63.68 | 6 |
| ATOM | 11807 | OG | SER | C | 1084 | 74.810 | 71.853 | 36.795 | 1.00 | 65.09 | 8 |
| ATOM | 11808 | C | SER | C | 1084 | 78.479 | 71.168 | 36.593 | 1.00 | 90.21 | 6 |
| ATOM | 11809 | O | SER | C | 1084 | 79.224 | 71.583 | 35.706 | 1.00 | 90.86 | 8 |
| ATOM | 11810 | N | PHE | C | 1085 | 78.903 | 70.873 | 37.819 | 1.00 | 103.08 | 7 |
| ATOM | 11811 | CA | PHE | C | 1085 | 80.304 | 71.045 | 38.209 | 1.00 | 100.39 | 6 |
| ATOM | 11812 | CB | PHE | C | 1085 | 80.544 | 70.506 | 39.623 | 1.00 | 70.97 | 6 |
| ATOM | 11813 | CG | PHE | C | 1085 | 81.844 | 70.954 | 40.245 | 1.00 | 71.55 | 6 |
| ATOM | 11814 | CD1 | PHE | C | 1085 | 82.457 | 72.134 | 39.849 | 1.00 | 71.72 | 6 |
| ATOM | 11815 | CD2 | PHE | C | 1085 | 82.427 | 70.213 | 41.270 | 1.00 | 72.44 | 6 |
| ATOM | 11816 | CE1 | PHE | C | 1085 | 83.629 | 72.563 | 40.466 | 1.00 | 71.44 | 6 |
| ATOM | 11817 | CE2 | PHE | C | 1085 | 83.597 | 70.637 | 41.890 | 1.00 | 71.86 | 6 |
| ATOM | 11818 | CZ | PHE | C | 1085 | 84.197 | 71.811 | 41.489 | 1.00 | 71.39 | 6 |
| ATOM | 11819 | C | PHE | C | 1085 | 81.195 | 70.314 | 37.234 | 1.00 | 98.75 | 6 |
| ATOM | 11820 | O | PHE | C | 1085 | 82.272 | 70.796 | 36.894 | 1.00 | 100.33 | 8 |
| ATOM | 11821 | N | ALA | C | 1086 | 80.729 | 69.154 | 36.782 | 1.00 | 37.35 | 7 |
| ATOM | 11822 | CA | ALA | C | 1086 | 81.484 | 68.344 | 35.838 | 1.00 | 34.90 | 6 |
| ATOM | 11823 | CB | ALA | C | 1086 | 80.845 | 66.966 | 35.714 | 1.00 | 13.87 | 6 |
| ATOM | 11824 | C | ALA | C | 1086 | 81.615 | 69.002 | 34.454 | 1.00 | 33.37 | 6 |
| ATOM | 11825 | O | ALA | C | 1086 | 82.555 | 68.729 | 33.708 | 1.00 | 34.08 | 8 |
| ATOM | 11826 | N | VAL | C | 1087 | 80.690 | 69.885 | 34.108 | 1.00 | 36.24 | 7 |
| ATOM | 11827 | CA | VAL | C | 1087 | 80.763 | 70.535 | 32.813 | 1.00 | 35.38 | 6 |
| ATOM | 11828 | CB | VAL | C | 1087 | 79.392 | 70.536 | 32.122 | 1.00 | 46.83 | 6 |
| ATOM | 11829 | CG1 | VAL | C | 1087 | 79.498 | 71.144 | 30.741 | 1.00 | 46.97 | 6 |
| ATOM | 11830 | CG2 | VAL | C | 1087 | 78.877 | 69.122 | 32.026 | 1.00 | 47.66 | 6 |
| ATOM | 11831 | C | VAL | C | 1087 | 81.282 | 71.958 | 32.930 | 1.00 | 34.55 | 6 |
| ATOM | 11832 | O | VAL | C | 1087 | 81.422 | 72.652 | 31.929 | 1.00 | 33.81 | 8 |
| ATOM | 11833 | N | LEU | C | 1088 | 81.570 | 72.406 | 34.145 | 1.00 | 98.32 | 7 |
| ATOM | 11834 | CA | LEU | C | 1088 | 82.097 | 73.753 | 34.290 | 1.00 | 98.34 | 6 |
| ATOM | 11835 | CB | LEU | C | 1088 | 81.884 | 74.307 | 35.699 | 1.00 | 20.30 | 6 |
| ATOM | 11836 | CG | LEU | C | 1088 | 82.332 | 75.769 | 35.855 | 1.00 | 17.77 | 6 |
| ATOM | 11837 | CD1 | LEU | C | 1088 | 81.606 | 76.670 | 34.873 | 1.00 | 17.28 | 6 |
| ATOM | 11838 | CD2 | LEU | C | 1088 | 82.065 | 76.227 | 37.262 | 1.00 | 18.02 | 6 |
| ATOM | 11839 | C | LEU | C | 1088 | 83.582 | 73.689 | 34.009 | 1.00 | 99.79 | 6 |
| ATOM | 11840 | O | LEU | C | 1088 | 84.134 | 74.546 | 33.323 | 1.00 | 101.96 | 8 |
| ATOM | 11841 | N | VAL | C | 1089 | 84.229 | 72.659 | 34.539 | 1.00 | 79.95 | 7 |
| ATOM | 11842 | CA | VAL | C | 1089 | 85.655 | 72.507 | 34.336 | 1.00 | 80.12 | 6 |
| ATOM | 11843 | CB | VAL | C | 1089 | 86.213 | 71.335 | 35.175 | 1.00 | 62.80 | 6 |
| ATOM | 11844 | CG1 | VAL | C | 1089 | 87.723 | 71.294 | 35.082 | 1.00 | 65.03 | 6 |
| ATOM | 11845 | CG2 | VAL | C | 1089 | 85.806 | 71.500 | 36.616 | 1.00 | 62.52 | 6 |
| ATOM | 11846 | C | VAL | C | 1089 | 85.951 | 72.278 | 32.853 | 1.00 | 79.71 | 6 |
| ATOM | 11847 | O | VAL | C | 1089 | 86.784 | 72.976 | 32.272 | 1.00 | 79.69 | 8 |
| ATOM | 11848 | N | ALA | C | 1090 | 85.250 | 71.327 | 32.236 | 1.00 | 80.39 | 7 |
| ATOM | 11849 | CA | ALA | C | 1090 | 85.479 | 71.010 | 30.827 | 1.00 | 80.44 | 6 |
| ATOM | 11850 | CB | ALA | C | 1090 | 84.991 | 69.592 | 30.515 | 1.00 | 13.87 | 6 |
| ATOM | 11851 | C | ALA | C | 1090 | 84.870 | 72.005 | 29.850 | 1.00 | 81.27 | 6 |
| ATOM | 11852 | O | ALA | C | 1090 | 84.588 | 71.668 | 28.704 | 1.00 | 82.52 | 8 |
| ATOM | 11853 | N | GLU | C | 1091 | 84.666 | 73.232 | 30.311 | 1.00 | 26.75 | 7 |
| ATOM | 11854 | CA | GLU | C | 1091 | 84.126 | 74.305 | 29.469 | 1.00 | 28.33 | 6 |
| ATOM | 11855 | CB | GLU | C | 1091 | 82.728 | 74.712 | 29.921 | 1.00 | 76.31 | 6 |
| ATOM | 11856 | CG | GLU | C | 1091 | 81.608 | 74.061 | 29.146 | 1.00 | 77.14 | 6 |
| ATOM | 11857 | CD | GLU | C | 1091 | 80.259 | 74.579 | 29.576 | 1.00 | 77.62 | 6 |
| ATOM | 11858 | OE1 | GLU | C | 1091 | 80.114 | 75.815 | 29.672 | 1.00 | 77.82 | 8 |
| ATOM | 11859 | OE2 | GLU | C | 1091 | 79.351 | 73.758 | 29.813 | 1.00 | 78.87 | 8 |
| ATOM | 11860 | C | GLU | C | 1091 | 85.046 | 75.508 | 29.601 | 1.00 | 28.72 | 6 |
| ATOM | 11861 | O | GLU | C | 1091 | 84.984 | 76.452 | 28.810 | 1.00 | 27.52 | 8 |
| ATOM | 11862 | N | LEU | C | 1092 | 85.873 | 75.455 | 30.643 | 1.00 | 80.09 | 7 |
| ATOM | 11863 | CA | LEU | C | 1092 | 86.862 | 76.477 | 30.961 | 1.00 | 82.54 | 6 |
| ATOM | 11864 | CB | LEU | C | 1092 | 87.006 | 76.608 | 32.486 | 1.00 | 54.50 | 6 |
| ATOM | 11865 | CG | LEU | C | 1092 | 85.982 | 77.409 | 33.314 | 1.00 | 53.94 | 6 |
| ATOM | 11866 | CD1 | LEU | C | 1092 | 85.982 | 76.931 | 34.770 | 1.00 | 52.07 | 6 |
| ATOM | 11867 | CD2 | LEU | C | 1092 | 86.313 | 78.902 | 33.235 | 1.00 | 54.02 | 6 |
| ATOM | 11868 | C | LEU | C | 1092 | 88.173 | 75.989 | 30.342 | 1.00 | 84.64 | 6 |
| ATOM | 11869 | O | LEU | C | 1092 | 89.037 | 76.784 | 29.962 | 1.00 | 85.41 | 8 |
| ATOM | 11870 | N | GLN | C | 1093 | 88.300 | 74.666 | 30.244 | 1.00 | 69.69 | 7 |
| ATOM | 11871 | CA | GLN | C | 1093 | 89.478 | 74.027 | 29.659 | 1.00 | 70.75 | 6 |
| ATOM | 11872 | CB | GLN | C | 1093 | 89.419 | 72.507 | 29.858 | 1.00 | 73.27 | 6 |
| ATOM | 11873 | CG | GLN | C | 1093 | 89.389 | 72.057 | 31.307 | 1.00 | 75.10 | 6 |
| ATOM | 11874 | CD | GLN | C | 1093 | 89.788 | 70.603 | 31.471 | 1.00 | 76.28 | 6 |
| ATOM | 11875 | OE1 | GLN | C | 1093 | 89.255 | 69.723 | 30.801 | 1.00 | 78.19 | 8 |
| ATOM | 11876 | NE2 | GLN | C | 1093 | 90.721 | 70.343 | 32.375 | 1.00 | 77.68 | 7 |
| ATOM | 11877 | C | GLN | C | 1093 | 89.535 | 74.336 | 28.164 | 1.00 | 71.47 | 6 |
| ATOM | 11878 | O | GLN | C | 1093 | 90.607 | 74.397 | 27.555 | 1.00 | 71.73 | 8 |
| ATOM | 11879 | N | ALA | C | 1094 | 88.359 | 74.514 | 27.579 | 1.00 | 80.43 | 7 |
| ATOM | 11880 | CA | ALA | C | 1094 | 88.272 | 74.821 | 26.168 | 1.00 | 81.50 | 6 |
| ATOM | 11881 | CB | ALA | C | 1094 | 86.815 | 74.888 | 25.734 | 1.00 | 133.59 | 6 |
| ATOM | 11882 | C | ALA | C | 1094 | 88.956 | 76.156 | 25.937 | 1.00 | 81.84 | 6 |
| ATOM | 11883 | O | ALA | C | 1094 | 89.576 | 76.371 | 24.901 | 1.00 | 81.71 | 8 |
| ATOM | 11884 | N | LEU | C | 1095 | 88.843 | 77.052 | 26.910 | 1.00 | 68.21 | 7 |
| ATOM | 11885 | CA | LEU | C | 1095 | 89.458 | 78.362 | 26.795 | 1.00 | 69.70 | 6 |
| ATOM | 11886 | CB | LEU | C | 1095 | 88.677 | 79.389 | 27.608 | 1.00 | 115.57 | 6 |
| ATOM | 11887 | CG | LEU | C | 1095 | 87.746 | 80.297 | 26.801 | 1.00 | 115.82 | 6 |
| ATOM | 11888 | CD1 | LEU | C | 1095 | 86.714 | 79.464 | 26.081 | 1.00 | 115.43 | 6 |
| ATOM | 11889 | CD2 | LEU | C | 1095 | 87.074 | 81.305 | 27.729 | 1.00 | 117.08 | 6 |
| ATOM | 11890 | C | LEU | C | 1095 | 90.904 | 78.307 | 27.265 | 1.00 | 70.83 | 6 |
| ATOM | 11891 | O | LEU | C | 1095 | 91.561 | 79.335 | 27.443 | 1.00 | 71.86 | 8 |
| ATOM | 11892 | N | ALA | C | 1096 | 91.394 | 77.090 | 27.460 | 1.00 | 118.71 | 7 |
| ATOM | 11893 | CA | ALA | C | 1096 | 92.768 | 76.880 | 27.887 | 1.00 | 119.87 | 6 |
| ATOM | 11894 | CB | ALA | C | 1096 | 93.721 | 77.602 | 26.936 | 1.00 | 94.68 | 6 |
| ATOM | 11895 | C | ALA | C | 1096 | 93.011 | 77.340 | 29.319 | 1.00 | 120.32 | 6 |
| ATOM | 11896 | O | ALA | C | 1096 | 93.966 | 78.068 | 29.589 | 1.00 | 120.38 | 8 |
| ATOM | 11897 | N | LEU | C | 1097 | 92.153 | 76.911 | 30.237 | 1.00 | 50.53 | 7 |
| ATOM | 11898 | CA | LEU | C | 1097 | 92.308 | 77.281 | 31.634 | 1.00 | 51.98 | 6 |
| ATOM | 11899 | CB | LEU | C | 1097 | 91.129 | 78.148 | 32.082 | 1.00 | 68.25 | 6 |
| ATOM | 11900 | CG | LEU | C | 1097 | 90.549 | 79.187 | 31.113 | 1.00 | 70.05 | 6 |
| ATOM | 11901 | CD1 | LEU | C | 1097 | 89.371 | 79.874 | 31.765 | 1.00 | 70.81 | 6 |
| ATOM | 11902 | CD2 | LEU | C | 1097 | 91.586 | 80.214 | 30.738 | 1.00 | 70.47 | 6 |
| ATOM | 11903 | C | LEU | C | 1097 | 92.333 | 75.991 | 32.434 | 1.00 | 52.35 | 6 |
| ATOM | 11904 | O | LEU | C | 1097 | 91.460 | 75.151 | 32.259 | 1.00 | 53.22 | 8 |
| ATOM | 11905 | N | ASP | C | 1098 | 93.330 | 75.806 | 33.291 | 1.00 | 110.12 | 7 |
| ATOM | 11906 | CA | ASP | C | 1098 | 93.369 | 74.591 | 34.097 | 1.00 | 110.77 | 6 |
| ATOM | 11907 | CB | ASP | C | 1098 | 94.808 | 74.174 | 34.418 | 1.00 | 134.82 | 6 |
| ATOM | 11908 | CG | ASP | C | 1098 | 94.885 | 72.805 | 35.100 | 1.00 | 136.59 | 6 |
| ATOM | 11909 | OD1 | ASP | C | 1098 | 94.194 | 72.603 | 36.120 | 1.00 | 137.99 | 8 |
| ATOM | 11910 | OD2 | ASP | C | 1098 | 95.637 | 71.929 | 34.618 | 1.00 | 137.11 | 8 |
| ATOM | 11911 | C | ASP | C | 1098 | 92.618 | 74.887 | 35.389 | 1.00 | 109.91 | 6 |
| ATOM | 11912 | O | ASP | C | 1098 | 92.992 | 75.794 | 36.137 | 1.00 | 110.36 | 8 |
| ATOM | 11913 | N | VAL | C | 1099 | 91.554 | 74.132 | 35.647 | 1.00 | 34.21 | 7 |
| ATOM | 11914 | CA | VAL | C | 1099 | 90.763 | 74.347 | 36.848 | 1.00 | 33.94 | 6 |
| ATOM | 11915 | CB | VAL | C | 1099 | 89.252 | 74.325 | 36.530 | 1.00 | 110.48 | 6 |
| ATOM | 11916 | CG1 | VAL | C | 1099 | 88.458 | 74.607 | 37.780 | 1.00 | 111.53 | 6 |
| ATOM | 11917 | CG2 | VAL | C | 1099 | 88.925 | 75.361 | 35.470 | 1.00 | 110.23 | 6 |
| ATOM | 11918 | C | VAL | C | 1099 | 91.083 | 73.258 | 37.853 | 1.00 | 34.64 | 6 |
| ATOM | 11919 | O | VAL | C | 1099 | 90.225 | 72.460 | 38.225 | 1.00 | 35.09 | 8 |
| ATOM | 11920 | N | GLN | C | 1100 | 92.332 | 73.227 | 38.293 | 1.00 | 47.29 | 7 |
| ATOM | 11921 | CA | GLN | C | 1100 | 92.741 | 72.219 | 39.253 | 1.00 | 48.51 | 6 |
| ATOM | 11922 | CB | GLN | C | 1100 | 94.199 | 72.437 | 39.681 | 1.00 | 208.87 | 6 |
| ATOM | 11923 | CG | GLN | C | 1100 | 95.220 | 71.846 | 38.706 | 1.00 | 208.87 | 6 |
| ATOM | 11924 | CD | GLN | C | 1100 | 96.653 | 71.976 | 39.195 | 1.00 | 208.87 | 6 |
| ATOM | 11925 | OE1 | GLN | C | 1100 | 96.973 | 71.603 | 40.325 | 1.00 | 208.87 | 8 |
| ATOM | 11926 | NE2 | GLN | C | 1100 | 97.527 | 72.493 | 38.339 | 1.00 | 208.87 | 7 |
| ATOM | 11927 | C | GLN | C | 1100 | 91.823 | 72.217 | 40.462 | 1.00 | 47.63 | 6 |
| ATOM | 11928 | O | GLN | C | 1100 | 91.685 | 73.213 | 41.158 | 1.00 | 47.48 | 8 |
| ATOM | 11929 | N | THR | C | 1101 | 91.190 | 71.082 | 40.700 | 1.00 | 68.00 | 7 |
| ATOM | 11930 | CA | THR | C | 1101 | 90.286 | 70.942 | 41.820 | 1.00 | 67.53 | 6 |
| ATOM | 11931 | CB | THR | C | 1101 | 89.096 | 70.076 | 41.412 | 1.00 | 37.08 | 6 |
| ATOM | 11932 | OG1 | THR | C | 1101 | 88.482 | 70.651 | 40.250 | 1.00 | 37.01 | 8 |
| ATOM | 11933 | CG2 | THR | C | 1101 | 88.076 | 69.995 | 42.539 | 1.00 | 36.67 | 6 |
| ATOM | 11934 | C | THR | C | 1101 | 91.028 | 70.307 | 42.991 | 1.00 | 67.60 | 6 |
| ATOM | 11935 | O | THR | C | 1101 | 91.610 | 69.240 | 42.850 | 1.00 | 68.10 | 8 |
| ATOM | 11936 | N | ALA | C | 1102 | 91.016 | 70.972 | 44.143 | 1.00 | 69.32 | 7 |
| ATOM | 11937 | CA | ALA | C | 1102 | 91.705 | 70.460 | 45.326 | 1.00 | 69.08 | 6 |
| ATOM | 11938 | CB | ALA | C | 1102 | 92.779 | 71.461 | 45.768 | 1.00 | 31.96 | 6 |
| ATOM | 11939 | C | ALA | C | 1102 | 90.766 | 70.132 | 46.500 | 1.00 | 68.74 | 6 |
| ATOM | 11940 | O | ALA | C | 1102 | 89.731 | 70.779 | 46.698 | 1.00 | 67.72 | 8 |
| ATOM | 11941 | N | ASP | C | 1103 | 91.150 | 69.115 | 47.270 | 1.00 | 77.27 | 7 |
| ATOM | 11942 | CA | ASP | C | 1103 | 90.382 | 68.660 | 48.421 | 1.00 | 77.46 | 6 |
| ATOM | 11943 | CB | ASP | C | 1103 | 90.421 | 67.136 | 48.507 | 1.00 | 132.50 | 6 |
| ATOM | 11944 | CG | ASP | C | 1103 | 90.062 | 66.474 | 47.196 | 1.00 | 133.78 | 6 |
| ATOM | 11945 | OD1 | ASP | C | 1103 | 88.950 | 66.734 | 46.690 | 1.00 | 133.76 | 8 |
| ATOM | 11946 | OD2 | ASP | C | 1103 | 90.892 | 65.697 | 46.671 | 1.00 | 135.67 | 8 |
| ATOM | 11947 | C | ASP | C | 1103 | 90.928 | 69.243 | 49.715 | 1.00 | 77.10 | 6 |
| ATOM | 11948 | O | ASP | C | 1103 | 92.027 | 69.783 | 49.747 | 1.00 | 77.24 | 8 |
| ATOM | 11949 | N | GLU | C | 1104 | 90.140 | 69.120 | 50.776 | 1.00 | 42.97 | 7 |
| ATOM | 11950 | CA | GLU | C | 1104 | 90.485 | 69.617 | 52.102 | 1.00 | 43.55 | 6 |
| ATOM | 11951 | CB | GLU | C | 1104 | 90.324 | 68.490 | 53.128 | 1.00 | 98.33 | 6 |
| ATOM | 11952 | CG | GLU | C | 1104 | 88.952 | 67.824 | 53.117 | 1.00 | 99.43 | 6 |
| ATOM | 11953 | CD | GLU | C | 1104 | 89.004 | 66.358 | 53.521 | 1.00 | 99.94 | 6 |
| ATOM | 11954 | OE1 | GLU | C | 1104 | 89.445 | 66.060 | 54.652 | 1.00 | 99.93 | 8 |
| ATOM | 11955 | OE2 | GLU | C | 1104 | 88.602 | 65.502 | 52.699 | 1.00 | 100.38 | 8 |
| ATOM | 11956 | C | GLU | C | 1104 | 91.900 | 70.158 | 52.174 | 1.00 | 44.00 | 6 |
| ATOM | 11957 | O | GLU | C | 1104 | 92.115 | 71.361 | 52.119 | 1.00 | 43.19 | 8 |
| ATOM | 11958 | N | ALA | C | 1105 | 92.858 | 69.242 | 52.267 | 1.00 | 86.07 | 7 |
| ATOM | 11959 | CA | ALA | C | 1105 | 94.272 | 69.570 | 52.388 | 1.00 | 88.23 | 6 |
| ATOM | 11960 | CB | ALA | C | 1105 | 95.068 | 68.291 | 52.644 | 1.00 | 96.16 | 6 |
| ATOM | 11961 | C | ALA | C | 1105 | 94.911 | 70.352 | 51.242 | 1.00 | 89.73 | 6 |
| ATOM | 11962 | O | ALA | C | 1105 | 96.135 | 70.408 | 51.153 | 1.00 | 90.32 | 8 |
| ATOM | 11963 | N | ASP | C | 1106 | 94.099 | 70.947 | 50.373 | 1.00 | 86.54 | 7 |
| ATOM | 11964 | CA | ASP | C | 1106 | 94.603 | 71.743 | 49.246 | 1.00 | 88.53 | 6 |
| ATOM | 11965 | CB | ASP | C | 1106 | 95.490 | 72.881 | 49.763 | 1.00 | 102.54 | 6 |
| ATOM | 11966 | CG | ASP | C | 1106 | 95.689 | 73.985 | 48.738 | 1.00 | 104.21 | 6 |
| ATOM | 11967 | OD1 | ASP | C | 1106 | 95.933 | 73.683 | 47.548 | 1.00 | 104.19 | 8 |
| ATOM | 11968 | OD2 | ASP | C | 1106 | 95.608 | 75.165 | 49.135 | 1.00 | 105.52 | 8 |
| ATOM | 11969 | C | ASP | C | 1106 | 95.393 | 70.930 | 48.226 | 1.00 | 89.27 | 6 |
| ATOM | 11970 | O | ASP | C | 1106 | 95.259 | 71.115 | 47.022 | 1.00 | 89.36 | 8 |
| ATOM | 11971 | N | ASN | C | 1107 | 96.223 | 70.029 | 48.725 | 1.00 | 70.86 | 7 |
| ATOM | 11972 | CA | ASN | C | 1107 | 97.051 | 69.192 | 47.875 | 1.00 | 72.87 | 6 |
| ATOM | 11973 | CB | ASN | C | 1107 | 98.479 | 69.145 | 48.443 | 1.00 | 208.73 | 6 |
| ATOM | 11974 | CG | ASN | C | 1107 | 99.510 | 68.690 | 47.421 | 1.00 | 208.87 | 6 |
| ATOM | 11975 | OD1 | ASN | C | 1107 | 99.637 | 67.500 | 47.131 | 1.00 | 208.87 | 8 |
| ATOM | 11976 | ND2 | ASN | C | 1107 | 100.252 | 69.644 | 46.867 | 1.00 | 208.87 | 7 |
| ATOM | 11977 | C | ASN | C | 1107 | 96.506 | 67.770 | 47.684 | 1.00 | 74.17 | 6 |
| ATOM | 11978 | O | ASN | C | 1107 | 97.271 | 66.835 | 47.437 | 1.00 | 73.99 | 8 |
| ATOM | 11979 | N | PRO | C | 1108 | 95.185 | 67.574 | 47.852 | 1.00 | 193.02 | 7 |
| ATOM | 11980 | CD | PRO | C | 1108 | 94.241 | 68.298 | 48.713 | 1.00 | 135.94 | 6 |
| ATOM | 11981 | CA | PRO | C | 1108 | 94.694 | 66.211 | 47.643 | 1.00 | 193.97 | 6 |
| ATOM | 11982 | CB | PRO | C | 1108 | 93.555 | 66.082 | 48.658 | 1.00 | 135.66 | 6 |
| ATOM | 11983 | CG | PRO | C | 1108 | 93.788 | 67.206 | 49.627 | 1.00 | 136.16 | 6 |
| ATOM | 11984 | C | PRO | C | 1108 | 94.168 | 66.195 | 46.215 | 1.00 | 194.79 | 6 |
| ATOM | 11985 | O | PRO | C | 1108 | 93.570 | 65.218 | 45.772 | 1.00 | 196.01 | 8 |
| ATOM | 11986 | N | VAL | C | 1109 | 94.404 | 67.310 | 45.523 | 1.00 | 53.75 | 7 |
| ATOM | 11987 | CA | VAL | C | 1109 | 93.978 | 67.531 | 44.149 | 1.00 | 53.98 | 6 |
| ATOM | 11988 | CB | VAL | C | 1109 | 95.182 | 67.519 | 43.174 | 1.00 | 138.02 | 6 |
| ATOM | 11989 | CG1 | VAL | C | 1109 | 95.920 | 66.201 | 43.266 | 1.00 | 138.58 | 6 |
| ATOM | 11990 | CG2 | VAL | C | 1109 | 94.704 | 67.771 | 41.751 | 1.00 | 138.25 | 6 |
| ATOM | 11991 | C | VAL | C | 1109 | 92.928 | 66.530 | 43.689 | 1.00 | 54.50 | 6 |
| ATOM | 11992 | O | VAL | C | 1109 | 93.217 | 65.357 | 43.465 | 1.00 | 54.78 | 8 |
| ATOM | 11993 | N | ALA | C | 1110 | 91.698 | 67.016 | 43.561 | 1.00 | 156.36 | 7 |
| ATOM | 11994 | CA | ALA | C | 1110 | 90.553 | 66.221 | 43.135 | 1.00 | 156.93 | 6 |
| ATOM | 11995 | CB | ALA | C | 1110 | 89.468 | 67.132 | 42.642 | 1.00 | 33.55 | 6 |
| ATOM | 11996 | C | ALA | C | 1110 | 90.858 | 65.197 | 42.061 | 1.00 | 157.67 | 6 |
| ATOM | 11997 | O | ALA | C | 1110 | 91.844 | 65.307 | 41.334 | 1.00 | 158.25 | 8 |
| ATOM | 11998 | N | VAL | C | 1111 | 89.983 | 64.206 | 41.959 | 1.00 | 134.81 | 7 |
| ATOM | 11999 | CA | VAL | C | 1111 | 90.130 | 63.154 | 40.967 | 1.00 | 135.66 | 6 |
| ATOM | 12000 | CB | VAL | C | 1111 | 90.197 | 61.762 | 41.639 | 1.00 | 184.71 | 6 |
| ATOM | 12001 | CG1 | VAL | C | 1111 | 90.488 | 60.684 | 40.600 | 1.00 | 185.47 | 6 |
| ATOM | 12002 | CG2 | VAL | C | 1111 | 91.278 | 61.765 | 42.712 | 1.00 | 184.30 | 6 |
| ATOM | 12003 | C | VAL | C | 1111 | 88.936 | 63.219 | 40.021 | 1.00 | 136.00 | 6 |
| ATOM | 12004 | O | VAL | C | 1111 | 88.652 | 62.273 | 39.286 | 1.00 | 136.25 | 8 |
| ATOM | 12005 | N | PHE | C | 1112 | 88.248 | 64.357 | 40.042 | 1.00 | 208.87 | 7 |
| ATOM | 12006 | CA | PHE | C | 1112 | 87.077 | 64.584 | 39.198 | 1.00 | 208.87 | 6 |
| ATOM | 12007 | CB | PHE | C | 1112 | 86.715 | 66.066 | 39.199 | 1.00 | 108.43 | 6 |
| ATOM | 12008 | CG | PHE | C | 1112 | 85.570 | 66.397 | 40.084 | 1.00 | 109.00 | 6 |
| ATOM | 12009 | CD1 | PHE | C | 1112 | 85.651 | 66.184 | 41.452 | 1.00 | 108.81 | 6 |
| ATOM | 12010 | CD2 | PHE | C | 1112 | 84.395 | 66.901 | 39.548 | 1.00 | 109.81 | 6 |
| ATOM | 12011 | CE1 | PHE | C | 1112 | 84.569 | 66.465 | 42.281 | 1.00 | 109.57 | 6 |
| ATOM | 12012 | CE2 | PHE | C | 1112 | 83.306 | 67.185 | 40.364 | 1.00 | 110.53 | 6 |
| ATOM | 12013 | CZ | PHE | C | 1112 | 83.392 | 66.967 | 41.736 | 1.00 | 110.35 | 6 |
| ATOM | 12014 | C | PHE | C | 1112 | 87.214 | 64.119 | 37.758 | 1.00 | 208.87 | 6 |
| ATOM | 12015 | O | PHE | C | 1112 | 88.323 | 63.986 | 37.232 | 1.00 | 208.87 | 8 |
| ATOM | 12016 | N | GLU | C | 1113 | 86.066 | 63.890 | 37.126 | 1.00 | 148.57 | 7 |
| ATOM | 12017 | CA | GLU | C | 1113 | 86.011 | 63.437 | 35.744 | 1.00 | 147.45 | 6 |
| ATOM | 12018 | CB | GLU | C | 1113 | 86.270 | 64.612 | 34.795 | 1.00 | 68.95 | 6 |
| ATOM | 12019 | CG | GLU | C | 1113 | 85.067 | 65.521 | 34.634 | 1.00 | 66.74 | 6 |
| ATOM | 12020 | CD | GLU | C | 1113 | 85.388 | 66.774 | 33.864 | 1.00 | 67.07 | 6 |
| ATOM | 12021 | OE1 | GLU | C | 1113 | 85.761 | 66.675 | 32.674 | 1.00 | 68.90 | 8 |
| ATOM | 12022 | OE2 | GLU | C | 1113 | 85.272 | 67.865 | 34.458 | 1.00 | 66.89 | 8 |
| ATOM | 12023 | C | GLU | C | 1113 | 86.966 | 62.283 | 35.459 | 1.00 | 147.59 | 6 |
| ATOM | 12024 | O | GLU | C | 1113 | 87.214 | 61.933 | 34.306 | 1.00 | 147.97 | 8 |
| ATOM | 12025 | N | GLY | C | 1114 | 87.502 | 61.696 | 36.523 | 1.00 | 180.49 | 7 |
| ATOM | 12026 | CA | GLY | C | 1114 | 88.386 | 60.561 | 36.364 | 1.00 | 179.91 | 6 |
| ATOM | 12027 | C | GLY | C | 1114 | 87.451 | 59.414 | 36.050 | 1.00 | 179.79 | 6 |
| ATOM | 12028 | O | GLY | C | 1114 | 87.791 | 58.242 | 36.202 | 1.00 | 180.67 | 8 |
| ATOM | 12029 | N | LEU | C | 1115 | 86.251 | 59.788 | 35.613 | 1.00 | 166.38 | 7 |
| ATOM | 12030 | CA | LEU | C | 1115 | 85.181 | 58.864 | 35.260 | 1.00 | 165.01 | 6 |
| ATOM | 12031 | CB | LEU | C | 1115 | 85.577 | 58.003 | 34.054 | 1.00 | 69.73 | 6 |
| ATOM | 12032 | CG | LEU | C | 1115 | 86.075 | 58.719 | 32.790 | 1.00 | 68.63 | 6 |
| ATOM | 12033 | CD1 | LEU | C | 1115 | 85.550 | 57.976 | 31.573 | 1.00 | 67.50 | 6 |
| ATOM | 12034 | CD2 | LEU | C | 1115 | 85.604 | 60.162 | 32.749 | 1.00 | 68.11 | 6 |
| ATOM | 12035 | C | LEU | C | 1115 | 84.822 | 57.971 | 36.438 | 1.00 | 165.21 | 6 |
| ATOM | 12036 | O | LEU | C | 1115 | 83.697 | 58.111 | 36.959 | 1.00 | 165.67 | 8 |
| ATOM | 12037 | OXT | LEU | C | 1115 | 85.673 | 57.151 | 36.834 | 1.00 | 71.04 | 8 |
| ATOM | 12038 | CB | LYS | D | 3 | 88.513 | 62.331 | 48.194 | 1.00 | 118.47 | 6 |
| ATOM | 12039 | CG | LYS | D | 3 | 89.024 | 63.117 | 49.388 | 1.00 | 119.73 | 6 |
| ATOM | 12040 | CD | LYS | D | 3 | 90.540 | 63.004 | 49.475 | 1.00 | 120.29 | 6 |
| ATOM | 12041 | CE | LYS | D | 3 | 91.089 | 63.665 | 50.730 | 1.00 | 121.57 | 6 |
| ATOM | 12042 | NZ | LYS | D | 3 | 92.578 | 63.555 | 50.820 | 1.00 | 121.53 | 7 |
| ATOM | 12043 | C | LYS | D | 3 | 86.420 | 63.574 | 47.574 | 1.00 | 65.05 | 6 |
| ATOM | 12044 | O | LYS | D | 3 | 86.688 | 63.995 | 46.452 | 1.00 | 65.91 | 8 |
| ATOM | 12045 | N | LYS | D | 3 | 86.624 | 61.180 | 47.108 | 1.00 | 63.59 | 7 |
| ATOM | 12046 | CA | LYS | D | 3 | 86.990 | 62.249 | 48.074 | 1.00 | 64.77 | 6 |
| ATOM | 12047 | N | ALA | D | 4 | 85.627 | 64.228 | 48.411 | 1.00 | 63.09 | 7 |
| ATOM | 12048 | CA | ALA | D | 4 | 84.999 | 65.494 | 48.043 | 1.00 | 62.71 | 6 |
| ATOM | 12049 | CB | ALA | D | 4 | 83.970 | 65.899 | 49.098 | 1.00 | 171.20 | 6 |
| ATOM | 12050 | C | ALA | D | 4 | 86.019 | 66.588 | 47.887 | 1.00 | 62.16 | 6 |
| ATOM | 12051 | O | ALA | D | 4 | 87.126 | 66.492 | 48.405 | 1.00 | 60.59 | 8 |
| ATOM | 12052 | N | VAL | D | 5 | 85.630 | 67.635 | 47.174 | 1.00 | 88.37 | 7 |
| ATOM | 12053 | CA | VAL | D | 5 | 86.504 | 68.771 | 46.958 | 1.00 | 88.36 | 6 |
| ATOM | 12054 | CB | VAL | D | 5 | 86.353 | 69.327 | 45.523 | 1.00 | 97.05 | 6 |
| ATOM | 12055 | CG1 | VAL | D | 5 | 86.047 | 68.195 | 44.564 | 1.00 | 98.63 | 6 |
| ATOM | 12056 | CG2 | VAL | D | 5 | 85.267 | 70.376 | 45.467 | 1.00 | 96.87 | 6 |
| ATOM | 12057 | C | VAL | D | 5 | 86.123 | 69.854 | 47.967 | 1.00 | 87.93 | 6 |
| ATOM | 12058 | O | VAL | D | 5 | 85.135 | 69.723 | 48.693 | 1.00 | 89.18 | 8 |
| ATOM | 12059 | N | ARG | D | 6 | 86.916 | 70.919 | 48.016 | 1.00 | 73.00 | 7 |
| ATOM | 12060 | CA | ARG | D | 6 | 86.656 | 72.029 | 48.927 | 1.00 | 72.03 | 6 |
| ATOM | 12061 | CB | ARG | D | 6 | 87.532 | 71.919 | 50.170 | 1.00 | 108.72 | 6 |
| ATOM | 12062 | CG | ARG | D | 6 | 87.374 | 70.629 | 50.944 | 1.00 | 110.80 | 6 |
| ATOM | 12063 | CD | ARG | D | 6 | 86.008 | 70.481 | 51.594 | 1.00 | 111.86 | 6 |
| ATOM | 12064 | NE | ARG | D | 6 | 86.035 | 69.364 | 52.530 | 1.00 | 113.64 | 7 |
| ATOM | 12065 | CZ | ARG | D | 6 | 85.008 | 68.984 | 53.275 | 1.00 | 115.19 | 6 |
| ATOM | 12066 | NH1 | ARG | D | 6 | 83.856 | 69.633 | 53.194 | 1.00 | 116.42 | 7 |
| ATOM | 12067 | NH2 | ARG | D | 6 | 85.141 | 67.961 | 54.109 | 1.00 | 115.84 | 7 |
| ATOM | 12068 | C | ARG | D | 6 | 86.971 | 73.331 | 48.216 | 1.00 | 70.72 | 6 |
| ATOM | 12069 | O | ARG | D | 6 | 86.745 | 74.416 | 48.751 | 1.00 | 70.74 | 8 |
| ATOM | 12070 | N | ALA | D | 7 | 87.499 | 73.211 | 47.006 | 1.00 | 72.80 | 7 |
| ATOM | 12071 | CA | ALA | D | 7 | 87.850 | 74.375 | 46.221 | 1.00 | 71.37 | 6 |
| ATOM | 12072 | CB | ALA | D | 7 | 89.278 | 74.779 | 46.512 | 1.00 | 49.57 | 6 |
| ATOM | 12073 | C | ALA | D | 7 | 87.687 | 74.065 | 44.748 | 1.00 | 70.55 | 6 |
| ATOM | 12074 | O | ALA | D | 7 | 87.147 | 73.022 | 44.379 | 1.00 | 71.42 | 8 |
| ATOM | 12075 | N | VAL | D | 8 | 88.147 | 74.988 | 43.913 | 1.00 | 41.62 | 7 |
| ATOM | 12076 | CA | VAL | D | 8 | 88.076 | 74.824 | 42.473 | 1.00 | 39.89 | 6 |
| ATOM | 12077 | CB | VAL | D | 8 | 86.912 | 75.617 | 41.870 | 1.00 | 59.38 | 6 |
| ATOM | 12078 | CG1 | VAL | D | 8 | 86.998 | 75.615 | 40.369 | 1.00 | 58.63 | 6 |
| ATOM | 12079 | CG2 | VAL | D | 8 | 85.607 | 74.980 | 42.279 | 1.00 | 60.49 | 6 |
| ATOM | 12080 | C | VAL | D | 8 | 89.383 | 75.323 | 41.908 | 1.00 | 39.57 | 6 |
| ATOM | 12081 | O | VAL | D | 8 | 90.024 | 74.633 | 41.119 | 1.00 | 39.83 | 8 |
| ATOM | 12082 | N | ALA | D | 9 | 89.773 | 76.527 | 42.317 | 1.00 | 41.52 | 7 |
| ATOM | 12083 | CA | ALA | D | 9 | 91.031 | 77.126 | 41.880 | 1.00 | 41.38 | 6 |
| ATOM | 12084 | CB | ALA | D | 9 | 92.188 | 76.489 | 42.640 | 1.00 | 116.73 | 6 |
| ATOM | 12085 | C | ALA | D | 9 | 91.325 | 77.086 | 40.376 | 1.00 | 41.12 | 6 |
| ATOM | 12086 | O | ALA | D | 9 | 91.858 | 76.100 | 39.852 | 1.00 | 40.67 | 8 |
| ATOM | 12087 | N | ILE | D | 10 | 90.979 | 78.175 | 39.695 | 1.00 | 55.36 | 7 |
| ATOM | 12088 | CA | ILE | D | 10 | 91.232 | 78.315 | 38.265 | 1.00 | 55.98 | 6 |
| ATOM | 12089 | CB | ILE | D | 10 | 90.593 | 79.618 | 37.711 | 1.00 | 52.97 | 6 |
| ATOM | 12090 | CG2 | ILE | D | 10 | 91.315 | 80.825 | 38.284 | 1.00 | 53.12 | 6 |
| ATOM | 12091 | CG1 | ILE | D | 10 | 90.667 | 79.658 | 36.182 | 1.00 | 52.23 | 6 |
| ATOM | 12092 | CD1 | ILE | D | 10 | 89.941 | 78.530 | 35.503 | 1.00 | 52.81 | 6 |
| ATOM | 12093 | C | ILE | D | 10 | 92.752 | 78.410 | 38.148 | 1.00 | 57.26 | 6 |
| ATOM | 12094 | O | ILE | D | 10 | 93.466 | 78.331 | 39.147 | 1.00 | 58.01 | 8 |
| ATOM | 12095 | N | ALA | D | 11 | 93.259 | 78.597 | 36.942 | 1.00 | 59.14 | 7 |
| ATOM | 12096 | CA | ALA | D | 11 | 94.696 | 78.681 | 36.782 | 1.00 | 60.37 | 6 |
| ATOM | 12097 | CB | ALA | D | 11 | 95.339 | 77.364 | 37.208 | 1.00 | 149.08 | 6 |
| ATOM | 12098 | C | ALA | D | 11 | 95.075 | 79.000 | 35.351 | 1.00 | 61.39 | 6 |
| ATOM | 12099 | O | ALA | D | 11 | 94.424 | 79.818 | 34.693 | 1.00 | 60.91 | 8 |
| ATOM | 12100 | N | LEU | D | 12 | 96.134 | 78.343 | 34.881 | 1.00 | 99.18 | 7 |
| ATOM | 12101 | CA | LEU | D | 12 | 96.629 | 78.540 | 33.530 | 1.00 | 99.22 | 6 |
| ATOM | 12102 | CB | LEU | D | 12 | 97.747 | 79.569 | 33.546 | 1.00 | 45.78 | 6 |
| ATOM | 12103 | CG | LEU | D | 12 | 97.829 | 80.343 | 32.238 | 1.00 | 45.71 | 6 |
| ATOM | 12104 | CD1 | LEU | D | 12 | 96.456 | 80.845 | 31.783 | 1.00 | 45.35 | 6 |
| ATOM | 12105 | CD2 | LEU | D | 12 | 98.767 | 81.492 | 32.463 | 1.00 | 46.63 | 6 |
| ATOM | 12106 | C | LEU | D | 12 | 97.119 | 77.221 | 32.939 | 1.00 | 99.44 | 6 |
| ATOM | 12107 | O | LEU | D | 12 | 97.801 | 76.445 | 33.614 | 1.00 | 99.97 | 8 |
| ATOM | 12108 | N | ALA | D | 13 | 96.773 | 76.984 | 31.673 | 1.00 | 88.25 | 7 |
| ATOM | 12109 | CA | ALA | D | 13 | 97.125 | 75.740 | 30.989 | 1.00 | 88.08 | 6 |
| ATOM | 12110 | CB | ALA | D | 13 | 95.862 | 75.096 | 30.431 | 1.00 | 145.76 | 6 |
| ATOM | 12111 | C | ALA | D | 13 | 98.177 | 75.838 | 29.886 | 1.00 | 87.96 | 6 |
| ATOM | 12112 | O | ALA | D | 13 | 97.905 | 76.324 | 28.785 | 1.00 | 87.79 | 8 |
| ATOM | 12113 | N | SER | D | 14 | 99.373 | 75.341 | 30.194 | 1.00 | 80.98 | 7 |
| ATOM | 12114 | CA | SER | D | 14 | 100.496 | 75.339 | 29.266 | 1.00 | 81.06 | 6 |
| ATOM | 12115 | CB | SER | D | 14 | 101.688 | 74.592 | 29.883 | 1.00 | 83.26 | 6 |
| ATOM | 12116 | OG | SER | D | 14 | 101.903 | 74.936 | 31.246 | 1.00 | 85.20 | 8 |
| ATOM | 12117 | C | SER | D | 14 | 100.054 | 74.615 | 28.002 | 1.00 | 81.18 | 6 |
| ATOM | 12118 | O | SER | D | 14 | 99.109 | 73.829 | 28.032 | 1.00 | 81.33 | 8 |
| ATOM | 12119 | N | PRO | D | 15 | 100.731 | 74.865 | 26.869 | 1.00 | 82.84 | 7 |
| ATOM | 12120 | CD | PRO | D | 15 | 102.017 | 75.557 | 26.673 | 1.00 | 117.32 | 6 |
| ATOM | 12121 | CA | PRO | D | 15 | 100.323 | 74.172 | 25.645 | 1.00 | 82.29 | 6 |
| ATOM | 12122 | CB | PRO | D | 15 | 101.270 | 74.739 | 24.600 | 1.00 | 116.72 | 6 |
| ATOM | 12123 | CG | PRO | D | 15 | 102.531 | 74.920 | 25.395 | 1.00 | 117.28 | 6 |
| ATOM | 12124 | C | PRO | D | 15 | 100.555 | 72.695 | 25.893 | 1.00 | 81.74 | 6 |
| ATOM | 12125 | O | PRO | D | 15 | 99.982 | 71.840 | 25.232 | 1.00 | 82.23 | 8 |
| ATOM | 12126 | N | ALA | D | 16 | 101.416 | 72.420 | 26.865 | 1.00 | 74.76 | 7 |
| ATOM | 12127 | CA | ALA | D | 16 | 101.736 | 71.060 | 27.251 | 1.00 | 74.09 | 6 |
| ATOM | 12128 | CB | ALA | D | 16 | 103.043 | 71.029 | 28.043 | 1.00 | 76.33 | 6 |
| ATOM | 12129 | C | ALA | D | 16 | 100.587 | 70.539 | 28.106 | 1.00 | 73.29 | 6 |
| ATOM | 12130 | O | ALA | D | 16 | 100.286 | 69.342 | 28.101 | 1.00 | 73.99 | 8 |
| ATOM | 12131 | N | ALA | D | 17 | 99.943 | 71.442 | 28.843 | 1.00 | 45.49 | 7 |
| ATOM | 12132 | CA | ALA | D | 17 | 98.827 | 71.043 | 29.693 | 1.00 | 43.80 | 6 |
| ATOM | 12133 | CB | ALA | D | 17 | 98.356 | 72.206 | 30.574 | 1.00 | 27.07 | 6 |
| ATOM | 12134 | C | ALA | D | 17 | 97.710 | 70.588 | 28.786 | 1.00 | 43.56 | 6 |
| ATOM | 12135 | O | ALA | D | 17 | 97.199 | 69.487 | 28.951 | 1.00 | 43.42 | 8 |
| ATOM | 12136 | N | ILE | D | 18 | 97.347 | 71.433 | 27.819 | 1.00 | 41.01 | 7 |
| ATOM | 12137 | CA | ILE | D | 18 | 96.284 | 71.117 | 26.865 | 1.00 | 42.69 | 6 |
| ATOM | 12138 | CB | ILE | D | 18 | 96.308 | 72.111 | 25.663 | 1.00 | 45.88 | 6 |
| ATOM | 12139 | CG2 | ILE | D | 18 | 95.676 | 71.494 | 24.440 | 1.00 | 46.21 | 6 |
| ATOM | 12140 | CG1 | ILE | D | 18 | 95.582 | 73.411 | 26.033 | 1.00 | 45.53 | 6 |
| ATOM | 12141 | CD1 | ILE | D | 18 | 96.333 | 74.321 | 26.988 | 1.00 | 43.91 | 6 |
| ATOM | 12142 | C | ILE | D | 18 | 96.440 | 69.674 | 26.368 | 1.00 | 44.77 | 6 |
| ATOM | 12143 | O | ILE | D | 18 | 95.489 | 68.885 | 26.413 | 1.00 | 44.47 | 8 |
| ATOM | 12144 | N | ARG | D | 19 | 97.651 | 69.337 | 25.923 | 1.00 | 69.82 | 7 |
| ATOM | 12145 | CA | ARG | D | 19 | 97.964 | 68.002 | 25.417 | 1.00 | 72.40 | 6 |
| ATOM | 12146 | CB | ARG | D | 19 | 99.464 | 67.866 | 25.129 | 1.00 | 92.42 | 6 |
| ATOM | 12147 | CG | ARG | D | 19 | 100.022 | 68.906 | 24.162 | 1.00 | 91.60 | 6 |
| ATOM | 12148 | CD | ARG | D | 19 | 101.551 | 68.848 | 24.057 | 1.00 | 90.18 | 6 |
| ATOM | 12149 | NE | ARG | D | 19 | 102.100 | 70.047 | 23.422 | 1.00 | 89.74 | 7 |
| ATOM | 12150 | CZ | ARG | D | 19 | 101.877 | 70.402 | 22.159 | 1.00 | 90.69 | 6 |
| ATOM | 12151 | NH1 | ARG | D | 19 | 101.116 | 69.647 | 21.377 | 1.00 | 91.13 | 7 |
| ATOM | 12152 | NH2 | ARG | D | 19 | 102.400 | 71.526 | 21.678 | 1.00 | 91.04 | 7 |
| ATOM | 12153 | C | ARG | D | 19 | 97.573 | 66.973 | 26.452 | 1.00 | 75.35 | 6 |
| ATOM | 12154 | O | ARG | D | 19 | 97.132 | 65.881 | 26.106 | 1.00 | 76.51 | 8 |
| ATOM | 12155 | N | ALA | D | 20 | 97.735 | 67.333 | 27.723 | 1.00 | 82.12 | 7 |
| ATOM | 12156 | CA | ALA | D | 20 | 97.413 | 66.438 | 28.832 | 1.00 | 86.47 | 6 |
| ATOM | 12157 | CB | ALA | D | 20 | 97.652 | 67.145 | 30.167 | 1.00 | 86.23 | 6 |
| ATOM | 12158 | C | ALA | D | 20 | 95.988 | 65.896 | 28.771 | 1.00 | 89.87 | 6 |
| ATOM | 12159 | O | ALA | D | 20 | 95.790 | 64.681 | 28.654 | 1.00 | 89.90 | 8 |
| ATOM | 12160 | N | TRP | D | 21 | 95.001 | 66.790 | 28.849 | 1.00 | 96.75 | 7 |
| ATOM | 12161 | CA | TRP | D | 21 | 93.604 | 66.367 | 28.805 | 1.00 | 100.95 | 6 |
| ATOM | 12162 | CB | TRP | D | 21 | 92.665 | 67.406 | 29.431 | 1.00 | 153.88 | 6 |
| ATOM | 12163 | CG | TRP | D | 21 | 93.301 | 68.494 | 30.227 | 1.00 | 155.88 | 6 |
| ATOM | 12164 | CD2 | TRP | D | 21 | 93.258 | 69.891 | 29.931 | 1.00 | 157.07 | 6 |
| ATOM | 12165 | CE2 | TRP | D | 21 | 93.935 | 70.561 | 30.971 | 1.00 | 158.45 | 6 |
| ATOM | 12166 | CE3 | TRP | D | 21 | 92.710 | 70.644 | 28.883 | 1.00 | 156.76 | 6 |
| ATOM | 12167 | CD1 | TRP | D | 21 | 93.986 | 68.371 | 31.401 | 1.00 | 157.18 | 6 |
| ATOM | 12168 | NE1 | TRP | D | 21 | 94.369 | 69.610 | 31.857 | 1.00 | 158.44 | 7 |
| ATOM | 12169 | CZ2 | TRP | D | 21 | 94.078 | 71.952 | 30.996 | 1.00 | 159.35 | 6 |
| ATOM | 12170 | CZ3 | TRP | D | 21 | 92.853 | 72.027 | 28.908 | 1.00 | 157.43 | 6 |
| ATOM | 12171 | CH2 | TRP | D | 21 | 93.532 | 72.666 | 29.958 | 1.00 | 158.88 | 6 |
| ATOM | 12172 | C | TRP | D | 21 | 93.140 | 66.123 | 27.379 | 1.00 | 103.62 | 6 |
| ATOM | 12173 | O | TRP | D | 21 | 92.388 | 65.187 | 27.115 | 1.00 | 104.42 | 8 |
| ATOM | 12174 | N | ALA | D | 22 | 93.585 | 66.980 | 26.467 | 1.00 | 208.87 | 7 |
| ATOM | 12175 | CA | ALA | D | 22 | 93.211 | 66.885 | 25.063 | 1.00 | 208.87 | 6 |
| ATOM | 12176 | CB | ALA | D | 22 | 94.115 | 67.773 | 24.242 | 1.00 | 126.20 | 6 |
| ATOM | 12177 | C | ALA | D | 22 | 93.233 | 65.456 | 24.518 | 1.00 | 208.87 | 6 |
| ATOM | 12178 | O | ALA | D | 22 | 92.762 | 65.197 | 23.409 | 1.00 | 208.87 | 8 |
| ATOM | 12179 | N | ALA | D | 23 | 93.781 | 64.531 | 25.297 | 1.00 | 191.58 | 7 |
| ATOM | 12180 | CA | ALA | D | 23 | 93.847 | 63.132 | 24.897 | 1.00 | 193.09 | 6 |
| ATOM | 12181 | CB | ALA | D | 23 | 92.431 | 62.549 | 24.795 | 1.00 | 74.47 | 6 |
| ATOM | 12182 | C | ALA | D | 23 | 94.586 | 62.946 | 23.578 | 1.00 | 194.46 | 6 |
| ATOM | 12183 | O | ALA | D | 23 | 94.602 | 61.846 | 23.029 | 1.00 | 195.08 | 8 |
| ATOM | 12184 | N | GLY | D | 24 | 95.193 | 64.021 | 23.076 | 1.00 | 208.87 | 7 |
| ATOM | 12185 | CA | GLY | D | 24 | 95.935 | 63.960 | 21.824 | 1.00 | 208.87 | 6 |
| ATOM | 12186 | C | GLY | D | 24 | 95.422 | 62.924 | 20.842 | 1.00 | 208.87 | 6 |
| ATOM | 12187 | O | GLY | D | 24 | 96.177 | 62.076 | 20.355 | 1.00 | 208.87 | 8 |
| ATOM | 12188 | N | ALA | D | 25 | 94.129 | 62.997 | 20.546 | 1.00 | 208.87 | 7 |
| ATOM | 12189 | CA | ALA | D | 25 | 93.505 | 62.053 | 19.634 | 1.00 | 208.87 | 6 |
| ATOM | 12190 | CB | ALA | D | 25 | 92.338 | 61.368 | 20.326 | 1.00 | 128.06 | 6 |
| ATOM | 12191 | C | ALA | D | 25 | 93.025 | 62.712 | 18.352 | 1.00 | 208.87 | 6 |
| ATOM | 12192 | O | ALA | D | 25 | 92.908 | 62.054 | 17.318 | 1.00 | 208.87 | 8 |
| ATOM | 12193 | N | ALA | D | 26 | 92.747 | 64.009 | 18.419 | 1.00 | 180.25 | 7 |
| ATOM | 12194 | CA | ALA | D | 26 | 92.260 | 64.726 | 17.249 | 1.00 | 180.10 | 6 |
| ATOM | 12195 | CB | ALA | D | 26 | 90.986 | 65.473 | 17.587 | 1.00 | 89.62 | 6 |
| ATOM | 12196 | C | ALA | D | 26 | 93.264 | 65.694 | 16.664 | 1.00 | 180.34 | 6 |
| ATOM | 12197 | O | ALA | D | 26 | 93.477 | 66.777 | 17.206 | 1.00 | 180.69 | 8 |
| ATOM | 12198 | N | GLU | D | 27 | 93.877 | 65.305 | 15.551 | 1.00 | 208.87 | 7 |
| ATOM | 12199 | CA | GLU | D | 27 | 94.825 | 66.180 | 14.879 | 1.00 | 208.87 | 6 |
| ATOM | 12200 | CB | GLU | D | 27 | 95.582 | 65.432 | 13.777 | 1.00 | 183.77 | 6 |
| ATOM | 12201 | CG | GLU | D | 27 | 96.682 | 66.265 | 13.127 | 1.00 | 184.01 | 6 |
| ATOM | 12202 | CD | GLU | D | 27 | 97.155 | 65.703 | 11.798 | 1.00 | 184.16 | 6 |
| ATOM | 12203 | OE1 | GLU | D | 27 | 97.663 | 64.563 | 11.769 | 1.00 | 184.40 | 8 |
| ATOM | 12204 | OE2 | GLU | D | 27 | 97.016 | 66.411 | 10.777 | 1.00 | 184.01 | 8 |
| ATOM | 12205 | C | GLU | D | 27 | 93.926 | 67.241 | 14.260 | 1.00 | 208.87 | 6 |
| ATOM | 12206 | O | GLU | D | 27 | 94.363 | 68.064 | 13.452 | 1.00 | 208.87 | 8 |
| ATOM | 12207 | N | ALA | D | 28 | 92.656 | 67.187 | 14.661 | 1.00 | 139.10 | 7 |
| ATOM | 12208 | CA | ALA | D | 28 | 91.616 | 68.098 | 14.206 | 1.00 | 138.27 | 6 |
| ATOM | 12209 | CB | ALA | D | 28 | 92.159 | 69.519 | 14.113 | 1.00 | 192.29 | 6 |
| ATOM | 12210 | C | ALA | D | 28 | 91.076 | 67.649 | 12.859 | 1.00 | 137.57 | 6 |
| ATOM | 12211 | O | ALA | D | 28 | 90.008 | 68.088 | 12.438 | 1.00 | 137.17 | 8 |
| ATOM | 12212 | N | ALA | D | 29 | 91.822 | 66.767 | 12.197 | 1.00 | 141.79 | 7 |
| ATOM | 12213 | CA | ALA | D | 29 | 91.445 | 66.241 | 10.889 | 1.00 | 140.97 | 6 |
| ATOM | 12214 | CB | ALA | D | 29 | 92.200 | 64.940 | 10.619 | 1.00 | 83.12 | 6 |
| ATOM | 12215 | C | ALA | D | 29 | 89.939 | 66.014 | 10.771 | 1.00 | 140.41 | 6 |
| ATOM | 12216 | O | ALA | D | 29 | 89.433 | 64.938 | 11.100 | 1.00 | 140.48 | 8 |
| ATOM | 12217 | N | ALA | D | 30 | 89.228 | 67.038 | 10.301 | 1.00 | 111.24 | 7 |
| ATOM | 12218 | CA | ALA | D | 30 | 87.780 | 66.963 | 10.136 | 1.00 | 110.84 | 6 |
| ATOM | 12219 | CB | ALA | D | 30 | 87.103 | 66.857 | 11.496 | 1.00 | 123.74 | 6 |
| ATOM | 12220 | C | ALA | D | 30 | 87.212 | 68.153 | 9.370 | 1.00 | 110.87 | 6 |
| ATOM | 12221 | O | ALA | D | 30 | 87.906 | 69.136 | 9.108 | 1.00 | 110.89 | 8 |
| ATOM | 12222 | N | ALA | D | 31 | 85.936 | 68.051 | 9.018 | 1.00 | 177.41 | 7 |
| ATOM | 12223 | CA | ALA | D | 31 | 85.248 | 69.102 | 8.283 | 1.00 | 177.79 | 6 |
| ATOM | 12224 | CB | ALA | D | 31 | 85.386 | 68.870 | 6.785 | 1.00 | 208.87 | 6 |
| ATOM | 12225 | C | ALA | D | 31 | 83.779 | 69.110 | 8.677 | 1.00 | 178.04 | 6 |
| ATOM | 12226 | O | ALA | D | 31 | 83.409 | 68.308 | 9.560 | 1.00 | 177.79 | 8 |
| ATOM | 12227 | OXT | ALA | D | 31 | 83.019 | 69.916 | 8.102 | 1.00 | 208.87 | 8 |
| ATOM | 12228 | CB | ALA | E | 69 | 80.302 | 61.345 | 2.995 | 1.00 | 182.41 | 6 |
| ATOM | 12229 | C | ALA | E | 69 | 79.572 | 61.683 | 5.364 | 1.00 | 208.52 | 6 |
| ATOM | 12230 | O | ALA | E | 69 | 78.367 | 61.590 | 5.600 | 1.00 | 208.79 | 8 |
| ATOM | 12231 | N | ALA | E | 69 | 79.514 | 59.422 | 4.332 | 1.00 | 208.62 | 7 |
| ATOM | 12232 | CA | ALA | E | 69 | 80.245 | 60.721 | 4.389 | 1.00 | 208.78 | 6 |
| ATOM | 12233 | N | ALA | E | 70 | 80.368 | 62.594 | 5.927 | 1.00 | 208.87 | 7 |
| ATOM | 12234 | CA | ALA | E | 70 | 79.908 | 63.616 | 6.875 | 1.00 | 208.87 | 6 |
| ATOM | 12235 | CB | ALA | E | 70 | 78.485 | 64.075 | 6.521 | 1.00 | 107.45 | 6 |
| ATOM | 12236 | C | ALA | E | 70 | 79.978 | 63.222 | 8.354 | 1.00 | 208.87 | 6 |
| ATOM | 12237 | O | ALA | E | 70 | 80.018 | 64.093 | 9.224 | 1.00 | 208.87 | 8 |
| ATOM | 12238 | N | ALA | E | 71 | 79.995 | 61.923 | 8.646 | 1.00 | 143.28 | 7 |
| ATOM | 12239 | CA | ALA | E | 71 | 80.070 | 61.471 | 10.035 | 1.00 | 141.93 | 6 |
| ATOM | 12240 | CB | ALA | E | 71 | 79.653 | 60.015 | 10.143 | 1.00 | 107.52 | 6 |
| ATOM | 12241 | C | ALA | E | 71 | 81.487 | 61.646 | 10.553 | 1.00 | 141.34 | 6 |
| ATOM | 12242 | O | ALA | E | 71 | 81.786 | 61.303 | 11.695 | 1.00 | 141.79 | 8 |
| ATOM | 12243 | N | ALA | E | 72 | 82.353 | 62.164 | 9.685 | 1.00 | 64.01 | 7 |
| ATOM | 12244 | CA | ALA | E | 72 | 83.751 | 62.429 | 10.004 | 1.00 | 63.37 | 6 |
| ATOM | 12245 | CB | ALA | E | 72 | 83.839 | 63.257 | 11.280 | 1.00 | 143.16 | 6 |
| ATOM | 12246 | C | ALA | E | 72 | 84.698 | 61.229 | 10.108 | 1.00 | 63.18 | 6 |
| ATOM | 12247 | O | ALA | E | 72 | 84.348 | 60.154 | 10.601 | 1.00 | 62.80 | 8 |
| ATOM | 12248 | N | ALA | E | 73 | 85.915 | 61.453 | 9.628 | 1.00 | 142.28 | 7 |
| ATOM | 12249 | CA | ALA | E | 73 | 86.979 | 60.463 | 9.659 | 1.00 | 142.92 | 6 |
| ATOM | 12250 | CB | ALA | E | 73 | 87.570 | 60.284 | 8.267 | 1.00 | 109.50 | 6 |
| ATOM | 12251 | C | ALA | E | 73 | 88.011 | 61.064 | 10.603 | 1.00 | 143.50 | 6 |
| ATOM | 12252 | O | ALA | E | 73 | 88.364 | 62.238 | 10.468 | 1.00 | 144.20 | 8 |
| ATOM | 12253 | N | ALA | E | 74 | 88.497 | 60.275 | 11.557 | 1.00 | 97.91 | 7 |
| ATOM | 12254 | CA | ALA | E | 74 | 89.461 | 60.793 | 12.523 | 1.00 | 98.24 | 6 |
| ATOM | 12255 | CB | ALA | E | 74 | 90.786 | 61.098 | 11.839 | 1.00 | 175.06 | 6 |
| ATOM | 12256 | C | ALA | E | 74 | 88.848 | 62.070 | 13.101 | 1.00 | 98.48 | 6 |
| ATOM | 12257 | O | ALA | E | 74 | 89.434 | 63.153 | 13.029 | 1.00 | 98.61 | 8 |
| ATOM | 12258 | N | ALA | E | 75 | 87.643 | 61.923 | 13.653 | 1.00 | 172.01 | 7 |
| ATOM | 12259 | CA | ALA | E | 75 | 86.902 | 63.032 | 14.249 | 1.00 | 172.44 | 6 |
| ATOM | 12260 | CB | ALA | E | 75 | 86.783 | 64.175 | 13.246 | 1.00 | 96.09 | 6 |
| ATOM | 12261 | C | ALA | E | 75 | 85.509 | 62.579 | 14.685 | 1.00 | 172.83 | 6 |
| ATOM | 12262 | O | ALA | E | 75 | 85.037 | 61.516 | 14.281 | 1.00 | 172.95 | 8 |
| ATOM | 12263 | N | ALA | E | 76 | 84.866 | 63.397 | 15.515 | 1.00 | 85.70 | 7 |
| ATOM | 12264 | CA | ALA | E | 76 | 83.520 | 63.136 | 16.019 | 1.00 | 85.45 | 6 |
| ATOM | 12265 | CB | ALA | E | 76 | 82.505 | 63.323 | 14.902 | 1.00 | 203.01 | 6 |
| ATOM | 12266 | C | ALA | E | 76 | 83.344 | 61.763 | 16.652 | 1.00 | 85.07 | 6 |
| ATOM | 12267 | O | ALA | E | 76 | 82.236 | 61.400 | 17.041 | 1.00 | 84.31 | 8 |
| ATOM | 12268 | N | ALA | E | 77 | 84.433 | 61.004 | 16.755 | 1.00 | 121.96 | 7 |
| ATOM | 12269 | CA | ALA | E | 77 | 84.394 | 59.664 | 17.341 | 1.00 | 122.45 | 6 |
| ATOM | 12270 | CB | ALA | E | 77 | 83.399 | 58.797 | 16.587 | 1.00 | 143.70 | 6 |
| ATOM | 12271 | C | ALA | E | 77 | 85.770 | 59.011 | 17.317 | 1.00 | 122.59 | 6 |
| ATOM | 12272 | O | ALA | E | 77 | 86.629 | 59.392 | 16.522 | 1.00 | 122.76 | 8 |
| ATOM | 12273 | N | ALA | E | 78 | 85.957 | 58.019 | 18.187 | 1.00 | 143.65 | 7 |
| ATOM | 12274 | CA | ALA | E | 78 | 87.214 | 57.286 | 18.314 | 1.00 | 143.63 | 6 |
| ATOM | 12275 | CB | ALA | E | 78 | 87.736 | 56.879 | 16.938 | 1.00 | 174.88 | 6 |
| ATOM | 12276 | C | ALA | E | 78 | 88.255 | 58.129 | 19.053 | 1.00 | 143.46 | 6 |
| ATOM | 12277 | O | ALA | E | 78 | 89.155 | 57.594 | 19.704 | 1.00 | 143.75 | 8 |
| ATOM | 12278 | N | ALA | E | 79 | 88.115 | 59.450 | 18.949 | 1.00 | 120.63 | 7 |
| ATOM | 12279 | CA | ALA | E | 79 | 89.011 | 60.405 | 19.607 | 1.00 | 120.05 | 6 |
| ATOM | 12280 | CB | ALA | E | 79 | 89.515 | 61.440 | 18.592 | 1.00 | 107.86 | 6 |
| ATOM | 12281 | C | ALA | E | 79 | 88.253 | 61.106 | 20.738 | 1.00 | 119.78 | 6 |
| ATOM | 12282 | O | ALA | E | 79 | 88.852 | 61.741 | 21.604 | 1.00 | 119.41 | 8 |
| ATOM | 12283 | N | ALA | E | 80 | 86.930 | 60.978 | 20.703 | 1.00 | 149.30 | 7 |
| ATOM | 12284 | CA | ALA | E | 80 | 86.030 | 61.572 | 21.685 | 1.00 | 149.38 | 6 |
| ATOM | 12285 | CB | ALA | E | 80 | 85.506 | 62.924 | 21.152 | 1.00 | 48.26 | 6 |
| ATOM | 12286 | C | ALA | E | 80 | 84.870 | 60.587 | 21.875 | 1.00 | 149.79 | 6 |
| ATOM | 12287 | O | ALA | E | 80 | 83.705 | 60.961 | 21.751 | 1.00 | 150.37 | 8 |
| ATOM | 12288 | N | ALA | E | 81 | 85.210 | 59.333 | 22.173 | 1.00 | 175.59 | 7 |
| ATOM | 12289 | CA | ALA | E | 81 | 84.242 | 58.248 | 22.355 | 1.00 | 174.88 | 6 |
| ATOM | 12290 | CB | ALA | E | 81 | 83.090 | 58.682 | 23.232 | 1.00 | 102.84 | 6 |
| ATOM | 12291 | C | ALA | E | 81 | 83.710 | 57.826 | 21.002 | 1.00 | 174.79 | 6 |
| ATOM | 12292 | O | ALA | E | 81 | 84.461 | 57.772 | 20.030 | 1.00 | 175.14 | 8 |
| ATOM | 12293 | N | ALA | E | 82 | 82.411 | 57.541 | 20.932 | 1.00 | 208.39 | 7 |
| ATOM | 12294 | CA | ALA | E | 82 | 81.819 | 57.143 | 19.662 | 1.00 | 208.06 | 6 |
| ATOM | 12295 | CB | ALA | E | 82 | 82.598 | 55.974 | 19.083 | 1.00 | 140.64 | 6 |
| ATOM | 12296 | C | ALA | E | 82 | 80.336 | 56.795 | 19.683 | 1.00 | 207.58 | 6 |
| ATOM | 12297 | O | ALA | E | 82 | 79.872 | 56.028 | 20.522 | 1.00 | 206.90 | 8 |
| ATOM | 12298 | N | ALA | E | 83 | 79.609 | 57.366 | 18.727 | 1.00 | 128.47 | 7 |
| ATOM | 12299 | CA | ALA | E | 83 | 78.181 | 57.123 | 18.526 | 1.00 | 128.25 | 6 |
| ATOM | 12300 | CB | ALA | E | 83 | 78.002 | 55.788 | 17.802 | 1.00 | 139.10 | 6 |
| ATOM | 12301 | C | ALA | E | 83 | 77.237 | 57.183 | 19.734 | 1.00 | 128.40 | 6 |
| ATOM | 12302 | O | ALA | E | 83 | 76.021 | 57.207 | 19.553 | 1.00 | 128.17 | 8 |
| ATOM | 12303 | N | ALA | E | 84 | 77.774 | 57.199 | 20.951 | 1.00 | 208.87 | 7 |
| ATOM | 12304 | CA | ALA | E | 84 | 76.942 | 57.266 | 22.152 | 1.00 | 208.87 | 6 |
| ATOM | 12305 | CB | ALA | E | 84 | 75.840 | 56.175 | 22.099 | 1.00 | 40.04 | 6 |
| ATOM | 12306 | C | ALA | E | 84 | 77.761 | 57.143 | 23.446 | 1.00 | 208.87 | 6 |
| ATOM | 12307 | O | ALA | E | 84 | 79.000 | 57.130 | 23.411 | 1.00 | 208.87 | 8 |
| ATOM | 12308 | N | ALA | E | 85 | 77.035 | 57.048 | 24.567 | 1.00 | 144.51 | 7 |
| ATOM | 12309 | CA | ALA | E | 85 | 77.561 | 56.942 | 25.941 | 1.00 | 143.76 | 6 |
| ATOM | 12310 | CB | ALA | E | 85 | 78.902 | 56.191 | 25.964 | 1.00 | 135.85 | 6 |
| ATOM | 12311 | C | ALA | E | 85 | 77.726 | 58.355 | 26.502 | 1.00 | 143.45 | 6 |
| ATOM | 12312 | O | ALA | E | 85 | 77.879 | 59.302 | 25.732 | 1.00 | 143.42 | 8 |
| ATOM | 12313 | N | ALA | E | 86 | 77.675 | 58.495 | 27.827 | 1.00 | 208.87 | 7 |
| ATOM | 12314 | CA | ALA | E | 86 | 77.817 | 59.792 | 28.498 | 1.00 | 208.87 | 6 |
| ATOM | 12315 | CB | ALA | E | 86 | 78.391 | 59.596 | 29.899 | 1.00 | 116.43 | 6 |
| ATOM | 12316 | C | ALA | E | 86 | 78.679 | 60.793 | 27.736 | 1.00 | 208.87 | 6 |
| ATOM | 12317 | O | ALA | E | 86 | 78.210 | 61.463 | 26.816 | 1.00 | 208.87 | 8 |
| ATOM | 12318 | N | ALA | E | 87 | 79.947 | 60.901 | 28.115 | 1.00 | 117.66 | 7 |
| ATOM | 12319 | CA | ALA | E | 87 | 80.820 | 61.845 | 27.434 | 1.00 | 115.28 | 6 |
| ATOM | 12320 | CB | ALA | E | 87 | 80.333 | 63.261 | 27.681 | 1.00 | 199.23 | 6 |
| ATOM | 12321 | C | ALA | E | 87 | 82.277 | 61.719 | 27.842 | 1.00 | 113.56 | 6 |
| ATOM | 12322 | O | ALA | E | 87 | 82.892 | 60.678 | 27.633 | 1.00 | 113.09 | 8 |
| ATOM | 12323 | N | ALA | E | 88 | 82.820 | 62.781 | 28.432 | 1.00 | 77.12 | 7 |
| ATOM | 12324 | CA | ALA | E | 88 | 84.219 | 62.806 | 28.849 | 1.00 | 74.14 | 6 |
| ATOM | 12325 | CB | ALA | E | 88 | 84.552 | 61.595 | 29.713 | 1.00 | 49.92 | 6 |
| ATOM | 12326 | C | ALA | E | 88 | 85.060 | 62.795 | 27.584 | 1.00 | 72.75 | 6 |
| ATOM | 12327 | O | ALA | E | 88 | 84.512 | 62.787 | 26.486 | 1.00 | 72.56 | 8 |
| ATOM | 12328 | N | ALA | E | 89 | 86.380 | 62.807 | 27.741 | 1.00 | 101.01 | 7 |
| ATOM | 12329 | CA | ALA | E | 89 | 87.295 | 62.813 | 26.603 | 1.00 | 99.71 | 6 |
| ATOM | 12330 | CB | ALA | E | 89 | 86.644 | 62.084 | 25.421 | 1.00 | 13.87 | 6 |
| ATOM | 12331 | C | ALA | E | 89 | 87.612 | 64.276 | 26.247 | 1.00 | 98.29 | 6 |
| ATOM | 12332 | O | ALA | E | 89 | 87.189 | 65.177 | 26.963 | 1.00 | 99.51 | 8 |
| ATOM | 12333 | N | ALA | E | 90 | 88.352 | 64.526 | 25.165 | 1.00 | 50.46 | 7 |
| ATOM | 12334 | CA | ALA | E | 90 | 88.661 | 65.904 | 24.771 | 1.00 | 46.80 | 6 |
| ATOM | 12335 | CB | ALA | E | 90 | 89.499 | 66.568 | 25.856 | 1.00 | 36.40 | 6 |
| ATOM | 12336 | C | ALA | E | 90 | 89.344 | 66.039 | 23.403 | 1.00 | 44.15 | 6 |
| ATOM | 12337 | O | ALA | E | 90 | 89.836 | 65.066 | 22.854 | 1.00 | 43.39 | 8 |
| ATOM | 12338 | N | ALA | E | 91 | 89.370 | 67.251 | 22.854 | 1.00 | 67.98 | 7 |
| ATOM | 12339 | CA | ALA | E | 91 | 89.980 | 67.489 | 21.544 | 1.00 | 66.57 | 6 |
| ATOM | 12340 | CB | ALA | E | 91 | 89.032 | 68.300 | 20.698 | 1.00 | 35.47 | 6 |
| ATOM | 12341 | C | ALA | E | 91 | 91.343 | 68.191 | 21.645 | 1.00 | 65.25 | 6 |
| ATOM | 12342 | O | ALA | E | 91 | 91.499 | 69.115 | 22.440 | 1.00 | 64.94 | 8 |
| ATOM | 12343 | N | ALA | E | 92 | 92.315 | 67.777 | 20.825 | 1.00 | 58.67 | 7 |
| ATOM | 12344 | CA | ALA | E | 92 | 93.670 | 68.348 | 20.887 | 1.00 | 57.66 | 6 |
| ATOM | 12345 | CB | ALA | E | 92 | 94.676 | 67.247 | 21.312 | 1.00 | 13.87 | 6 |
| ATOM | 12346 | C | ALA | E | 92 | 94.239 | 69.071 | 19.669 | 1.00 | 57.31 | 6 |
| ATOM | 12347 | O | ALA | E | 92 | 95.452 | 68.999 | 19.460 | 1.00 | 57.49 | 8 |
| ATOM | 12348 | N | ALA | E | 93 | 93.415 | 69.774 | 18.889 | 1.00 | 47.40 | 7 |
| ATOM | 12349 | CA | ALA | E | 93 | 93.925 | 70.479 | 17.697 | 1.00 | 46.65 | 6 |
| ATOM | 12350 | CB | ALA | E | 93 | 92.923 | 71.557 | 17.234 | 1.00 | 13.87 | 6 |
| ATOM | 12351 | C | ALA | E | 93 | 95.302 | 71.101 | 17.980 | 1.00 | 46.65 | 6 |
| ATOM | 12352 | O | ALA | E | 93 | 95.393 | 72.209 | 18.513 | 1.00 | 46.91 | 8 |
| ATOM | 12353 | N | ALA | E | 94 | 96.359 | 70.367 | 17.611 | 1.00 | 34.26 | 7 |
| ATOM | 12354 | CA | ALA | E | 94 | 97.763 | 70.756 | 17.844 | 1.00 | 35.14 | 6 |
| ATOM | 12355 | CB | ALA | E | 94 | 98.699 | 69.539 | 17.598 | 1.00 | 36.50 | 6 |
| ATOM | 12356 | C | ALA | E | 94 | 98.287 | 71.979 | 17.089 | 1.00 | 36.41 | 6 |
| ATOM | 12357 | O | ALA | E | 94 | 99.498 | 72.190 | 17.012 | 1.00 | 34.77 | 8 |
| ATOM | 12358 | N | ALA | E | 95 | 97.367 | 72.788 | 16.566 | 1.00 | 101.08 | 7 |
| ATOM | 12359 | CA | ALA | E | 95 | 97.703 | 74.011 | 15.838 | 1.00 | 104.48 | 6 |
| ATOM | 12360 | CB | ALA | E | 95 | 98.002 | 75.154 | 16.844 | 1.00 | 54.02 | 6 |
| ATOM | 12361 | C | ALA | E | 95 | 98.897 | 73.782 | 14.917 | 1.00 | 106.91 | 6 |
| ATOM | 12362 | O | ALA | E | 95 | 99.141 | 72.653 | 14.491 | 1.00 | 108.92 | 8 |
| ATOM | 12363 | N | ALA | E | 96 | 99.623 | 74.854 | 14.601 | 1.00 | 105.52 | 7 |
| ATOM | 12364 | CA | ALA | E | 96 | 100.809 | 74.764 | 13.748 | 1.00 | 107.06 | 6 |
| ATOM | 12365 | CB | ALA | E | 96 | 100.450 | 74.181 | 12.398 | 1.00 | 13.87 | 6 |
| ATOM | 12366 | C | ALA | E | 96 | 101.514 | 76.099 | 13.553 | 1.00 | 108.62 | 6 |
| ATOM | 12367 | O | ALA | E | 96 | 101.116 | 76.910 | 12.715 | 1.00 | 108.93 | 8 |
| ATOM | 12368 | N | ALA | E | 97 | 102.567 | 76.309 | 14.336 | 1.00 | 128.48 | 7 |
| ATOM | 12369 | CA | ALA | E | 97 | 103.372 | 77.521 | 14.266 | 1.00 | 129.82 | 6 |
| ATOM | 12370 | CB | ALA | E | 97 | 104.147 | 77.543 | 12.958 | 1.00 | 134.57 | 6 |
| ATOM | 12371 | C | ALA | E | 97 | 102.559 | 78.802 | 14.406 | 1.00 | 130.98 | 6 |
| ATOM | 12372 | O | ALA | E | 97 | 102.624 | 79.685 | 13.551 | 1.00 | 130.80 | 8 |
| ATOM | 12373 | N | ALA | E | 98 | 101.802 | 78.907 | 15.493 | 1.00 | 116.28 | 7 |
| ATOM | 12374 | CA | ALA | E | 98 | 100.989 | 80.091 | 15.727 | 1.00 | 117.99 | 6 |
| ATOM | 12375 | CB | ALA | E | 98 | 99.826 | 80.113 | 14.752 | 1.00 | 87.41 | 6 |
| ATOM | 12376 | C | ALA | E | 98 | 100.481 | 80.186 | 17.164 | 1.00 | 119.35 | 6 |
| ATOM | 12377 | O | ALA | E | 98 | 100.957 | 79.471 | 18.050 | 1.00 | 119.68 | 8 |
| ATOM | 12378 | N | ALA | E | 99 | 99.517 | 81.081 | 17.381 | 1.00 | 92.06 | 7 |
| ATOM | 12379 | CA | ALA | E | 99 | 98.921 | 81.312 | 18.700 | 1.00 | 93.57 | 6 |
| ATOM | 12380 | CB | ALA | E | 99 | 99.965 | 81.858 | 19.655 | 1.00 | 165.12 | 6 |
| ATOM | 12381 | C | ALA | E | 99 | 97.754 | 82.294 | 18.600 | 1.00 | 94.85 | 6 |
| ATOM | 12382 | O | ALA | E | 99 | 97.259 | 82.568 | 17.510 | 1.00 | 95.31 | 8 |
| ATOM | 12383 | N | ALA | E | 100 | 97.323 | 82.839 | 19.734 | 1.00 | 113.85 | 7 |
| ATOM | 12384 | CA | ALA | E | 100 | 96.203 | 83.772 | 19.730 | 1.00 | 114.80 | 6 |
| ATOM | 12385 | CB | ALA | E | 100 | 94.925 | 83.021 | 19.433 | 1.00 | 55.44 | 6 |
| ATOM | 12386 | C | ALA | E | 100 | 96.073 | 84.519 | 21.048 | 1.00 | 115.99 | 6 |
| ATOM | 12387 | O | ALA | E | 100 | 95.838 | 83.916 | 22.095 | 1.00 | 116.63 | 8 |
| ATOM | 12388 | N | ALA | E | 101 | 96.197 | 85.840 | 20.985 | 1.00 | 208.87 | 7 |
| ATOM | 12389 | CA | ALA | E | 101 | 96.120 | 86.677 | 22.178 | 1.00 | 208.87 | 6 |
| ATOM | 12390 | CB | ALA | E | 101 | 97.103 | 87.837 | 22.044 | 1.00 | 152.73 | 6 |
| ATOM | 12391 | C | ALA | E | 101 | 94.726 | 87.215 | 22.520 | 1.00 | 208.87 | 6 |
| ATOM | 12392 | O | ALA | E | 101 | 94.597 | 88.366 | 22.943 | 1.00 | 208.87 | 8 |
| ATOM | 12393 | N | ALA | E | 102 | 93.690 | 86.394 | 22.352 | 1.00 | 125.03 | 7 |
| ATOM | 12394 | CA | ALA | E | 102 | 92.326 | 86.833 | 22.657 | 1.00 | 124.04 | 6 |
| ATOM | 12395 | CB | ALA | E | 102 | 91.348 | 86.225 | 21.670 | 1.00 | 79.18 | 6 |
| ATOM | 12396 | C | ALA | E | 102 | 91.963 | 86.424 | 24.076 | 1.00 | 123.79 | 6 |
| ATOM | 12397 | O | ALA | E | 102 | 91.284 | 87.154 | 24.803 | 1.00 | 123.54 | 8 |
| ATOM | 12398 | N | ALA | E | 103 | 92.427 | 85.242 | 24.459 | 1.00 | 154.47 | 7 |
| ATOM | 12399 | CA | ALA | E | 103 | 92.178 | 84.717 | 25.787 | 1.00 | 153.98 | 6 |
| ATOM | 12400 | CB | ALA | E | 103 | 92.455 | 83.211 | 25.802 | 1.00 | 111.96 | 6 |
| ATOM | 12401 | C | ALA | E | 103 | 93.085 | 85.435 | 26.788 | 1.00 | 153.34 | 6 |
| ATOM | 12402 | O | ALA | E | 103 | 93.701 | 84.783 | 27.634 | 1.00 | 153.16 | 8 |
| ATOM | 12403 | N | ALA | E | 104 | 93.159 | 86.768 | 26.679 | 1.00 | 56.57 | 7 |
| ATOM | 12404 | CA | ALA | E | 104 | 93.991 | 87.622 | 27.547 | 1.00 | 55.34 | 6 |
| ATOM | 12405 | CB | ALA | E | 104 | 94.660 | 86.778 | 28.641 | 1.00 | 22.37 | 6 |
| ATOM | 12406 | C | ALA | E | 104 | 95.065 | 88.363 | 26.740 | 1.00 | 55.07 | 6 |
| ATOM | 12407 | O | ALA | E | 104 | 96.090 | 87.788 | 26.422 | 1.00 | 55.23 | 8 |
| ATOM | 12408 | N | ALA | E | 105 | 94.841 | 89.630 | 26.400 | 1.00 | 115.74 | 7 |
| ATOM | 12409 | CA | ALA | E | 105 | 95.837 | 90.379 | 25.624 | 1.00 | 114.78 | 6 |
| ATOM | 12410 | CB | ALA | E | 105 | 95.210 | 90.924 | 24.313 | 1.00 | 29.18 | 6 |
| ATOM | 12411 | C | ALA | E | 105 | 96.480 | 91.522 | 26.416 | 1.00 | 113.87 | 6 |
| ATOM | 12412 | O | ALA | E | 105 | 97.668 | 91.810 | 26.256 | 1.00 | 113.50 | 8 |
| ATOM | 12413 | N | ALA | E | 106 | 95.695 | 92.170 | 27.270 | 1.00 | 136.29 | 7 |
| ATOM | 12414 | CA | ALA | E | 106 | 96.192 | 93.281 | 28.078 | 1.00 | 135.70 | 6 |
| ATOM | 12415 | CB | ALA | E | 106 | 95.059 | 93.916 | 28.866 | 1.00 | 139.76 | 6 |
| ATOM | 12416 | C | ALA | E | 106 | 97.267 | 92.804 | 29.028 | 1.00 | 134.84 | 6 |
| ATOM | 12417 | O | ALA | E | 106 | 98.326 | 93.412 | 29.113 | 1.00 | 135.36 | 8 |
| ATOM | 12418 | N | ALA | E | 107 | 96.989 | 91.727 | 29.754 | 1.00 | 121.92 | 7 |
| ATOM | 12419 | CA | ALA | E | 107 | 97.972 | 91.191 | 30.681 | 1.00 | 121.18 | 6 |
| ATOM | 12420 | CB | ALA | E | 107 | 97.578 | 89.775 | 31.122 | 1.00 | 103.17 | 6 |
| ATOM | 12421 | C | ALA | E | 107 | 99.296 | 91.180 | 29.921 | 1.00 | 120.64 | 6 |
| ATOM | 12422 | O | ALA | E | 107 | 100.343 | 91.551 | 30.457 | 1.00 | 120.71 | 8 |
| ATOM | 12423 | N | ALA | E | 108 | 99.235 | 90.781 | 28.654 | 1.00 | 66.90 | 7 |
| ATOM | 12424 | CA | ALA | E | 108 | 100.422 | 90.746 | 27.811 | 1.00 | 66.00 | 6 |
| ATOM | 12425 | CB | ALA | E | 108 | 100.098 | 90.085 | 26.459 | 1.00 | 29.40 | 6 |
| ATOM | 12426 | C | ALA | E | 108 | 100.864 | 92.192 | 27.617 | 1.00 | 65.63 | 6 |
| ATOM | 12427 | O | ALA | E | 108 | 102.018 | 92.534 | 27.857 | 1.00 | 65.91 | 8 |
| ATOM | 12428 | N | ALA | E | 109 | 99.923 | 93.037 | 27.208 | 1.00 | 94.14 | 7 |
| ATOM | 12429 | CA | ALA | E | 109 | 100.189 | 94.455 | 26.982 | 1.00 | 94.04 | 6 |
| ATOM | 12430 | CB | ALA | E | 109 | 98.916 | 95.142 | 26.493 | 1.00 | 157.23 | 6 |
| ATOM | 12431 | C | ALA | E | 109 | 100.733 | 95.176 | 28.227 | 1.00 | 93.66 | 6 |
| ATOM | 12432 | O | ALA | E | 109 | 101.295 | 96.271 | 28.126 | 1.00 | 93.28 | 8 |
| ATOM | 12433 | N | ALA | E | 110 | 100.557 | 94.563 | 29.396 | 1.00 | 88.93 | 7 |
| ATOM | 12434 | CA | ALA | E | 110 | 101.036 | 95.142 | 30.647 | 1.00 | 87.68 | 6 |
| ATOM | 12435 | CB | ALA | E | 110 | 100.083 | 94.792 | 31.804 | 1.00 | 13.87 | 6 |
| ATOM | 12436 | C | ALA | E | 110 | 102.438 | 94.617 | 30.935 | 1.00 | 87.36 | 6 |
| ATOM | 12437 | O | ALA | E | 110 | 103.388 | 95.395 | 31.003 | 1.00 | 87.91 | 8 |
| ATOM | 12438 | N | ALA | E | 111 | 102.565 | 93.298 | 31.082 | 1.00 | 71.71 | 7 |
| ATOM | 12439 | CA | ALA | E | 111 | 103.855 | 92.667 | 31.363 | 1.00 | 72.01 | 6 |
| ATOM | 12440 | CB | ALA | E | 111 | 103.666 | 91.140 | 31.585 | 1.00 | 13.87 | 6 |
| ATOM | 12441 | C | ALA | E | 111 | 104.903 | 92.917 | 30.264 | 1.00 | 72.29 | 6 |
| ATOM | 12442 | O | ALA | E | 111 | 105.839 | 92.128 | 30.108 | 1.00 | 73.03 | 8 |
| ATOM | 12443 | N | ALA | E | 112 | 104.756 | 94.012 | 29.515 | 1.00 | 96.42 | 7 |
| ATOM | 12444 | CA | ALA | E | 112 | 105.695 | 94.347 | 28.440 | 1.00 | 95.42 | 6 |
| ATOM | 12445 | CB | ALA | E | 112 | 105.906 | 93.120 | 27.534 | 1.00 | 41.44 | 6 |
| ATOM | 12446 | C | ALA | E | 112 | 105.256 | 95.549 | 27.593 | 1.00 | 94.77 | 6 |
| ATOM | 12447 | O | ALA | E | 112 | 105.085 | 95.423 | 26.381 | 1.00 | 94.39 | 8 |
| ATOM | 12448 | N | ALA | E | 113 | 105.098 | 96.701 | 28.247 | 1.00 | 98.10 | 7 |
| ATOM | 12449 | CA | ALA | E | 113 | 104.679 | 97.981 | 27.644 | 1.00 | 98.34 | 6 |
| ATOM | 12450 | CB | ALA | E | 113 | 105.786 | 99.051 | 27.876 | 1.00 | 13.87 | 6 |
| ATOM | 12451 | C | ALA | E | 113 | 104.238 | 98.006 | 26.169 | 1.00 | 99.05 | 6 |
| ATOM | 12452 | O | ALA | E | 113 | 104.471 | 98.995 | 25.469 | 1.00 | 99.32 | 8 |
| ATOM | 12453 | N | ALA | E | 114 | 103.576 | 96.944 | 25.711 | 1.00 | 146.06 | 7 |
| ATOM | 12454 | CA | ALA | E | 114 | 103.117 | 96.853 | 24.323 | 1.00 | 146.52 | 6 |
| ATOM | 12455 | CB | ALA | E | 114 | 103.658 | 95.554 | 23.671 | 1.00 | 49.17 | 6 |
| ATOM | 12456 | C | ALA | E | 114 | 101.587 | 96.904 | 24.229 | 1.00 | 146.76 | 6 |
| ATOM | 12457 | O | ALA | E | 114 | 100.913 | 95.875 | 24.305 | 1.00 | 147.41 | 8 |
| ATOM | 12458 | N | ALA | E | 115 | 101.048 | 98.108 | 24.057 | 1.00 | 57.90 | 7 |
| ATOM | 12459 | CA | ALA | E | 115 | 99.601 | 98.305 | 23.959 | 1.00 | 56.95 | 6 |
| ATOM | 12460 | CB | ALA | E | 115 | 99.114 | 99.139 | 25.143 | 1.00 | 151.32 | 6 |
| ATOM | 12461 | C | ALA | E | 115 | 99.193 | 98.976 | 22.646 | 1.00 | 56.60 | 6 |
| ATOM | 12462 | O | ALA | E | 115 | 99.868 | 99.892 | 22.170 | 1.00 | 55.48 | 8 |
| ATOM | 12463 | N | ALA | E | 116 | 98.078 | 98.532 | 22.071 | 1.00 | 112.70 | 7 |
| ATOM | 12464 | CA | ALA | E | 116 | 97.609 | 99.083 | 20.803 | 1.00 | 113.68 | 6 |
| ATOM | 12465 | CB | ALA | E | 116 | 97.489 | 100.606 | 20.886 | 1.00 | 45.97 | 6 |
| ATOM | 12466 | C | ALA | E | 116 | 98.645 | 98.690 | 19.755 | 1.00 | 114.23 | 6 |
| ATOM | 12467 | O | ALA | E | 116 | 98.450 | 98.875 | 18.551 | 1.00 | 114.46 | 8 |
| ATOM | 12468 | N | ALA | E | 117 | 99.753 | 98.141 | 20.244 | 1.00 | 118.77 | 7 |
| ATOM | 12469 | CA | ALA | E | 117 | 100.852 | 97.691 | 19.408 | 1.00 | 118.20 | 6 |
| ATOM | 12470 | CB | ALA | E | 117 | 102.183 | 98.081 | 20.044 | 1.00 | 100.22 | 6 |
| ATOM | 12471 | C | ALA | E | 117 | 100.751 | 96.177 | 19.274 | 1.00 | 117.69 | 6 |
| ATOM | 12472 | O | ALA | E | 117 | 100.983 | 95.627 | 18.199 | 1.00 | 118.09 | 8 |
| ATOM | 12473 | N | ALA | E | 118 | 100.401 | 95.507 | 20.370 | 1.00 | 121.35 | 7 |
| ATOM | 12474 | CA | ALA | E | 118 | 100.255 | 94.054 | 20.360 | 1.00 | 120.31 | 6 |
| ATOM | 12475 | CB | ALA | E | 118 | 99.940 | 93.534 | 21.765 | 1.00 | 41.23 | 6 |
| ATOM | 12476 | C | ALA | E | 118 | 99.116 | 93.735 | 19.403 | 1.00 | 119.61 | 6 |
| ATOM | 12477 | O | ALA | E | 118 | 98.784 | 92.575 | 19.163 | 1.00 | 119.51 | 8 |
| ATOM | 12478 | N | ALA | E | 119 | 98.518 | 94.791 | 18.866 | 1.00 | 131.56 | 7 |
| ATOM | 12479 | CA | ALA | E | 119 | 97.438 | 94.658 | 17.910 | 1.00 | 130.77 | 6 |
| ATOM | 12480 | CB | ALA | E | 119 | 96.497 | 95.832 | 18.028 | 1.00 | 93.87 | 6 |
| ATOM | 12481 | C | ALA | E | 119 | 98.082 | 94.631 | 16.531 | 1.00 | 129.80 | 6 |
| ATOM | 12482 | O | ALA | E | 119 | 97.480 | 94.193 | 15.553 | 1.00 | 130.43 | 8 |
| ATOM | 12483 | N | ALA | E | 120 | 99.319 | 95.108 | 16.467 | 1.00 | 75.06 | 7 |
| ATOM | 12484 | CA | ALA | E | 120 | 100.072 | 95.135 | 15.222 | 1.00 | 72.98 | 6 |
| ATOM | 12485 | CB | ALA | E | 120 | 101.078 | 96.281 | 15.257 | 1.00 | 83.58 | 6 |
| ATOM | 12486 | C | ALA | E | 120 | 100.798 | 93.802 | 15.023 | 1.00 | 71.14 | 6 |
| ATOM | 12487 | O | ALA | E | 120 | 100.532 | 93.069 | 14.069 | 1.00 | 70.20 | 8 |
| ATOM | 12488 | N | ALA | E | 121 | 101.704 | 93.501 | 15.948 | 1.00 | 49.75 | 7 |
| ATOM | 12489 | CA | ALA | E | 121 | 102.511 | 92.280 | 15.924 | 1.00 | 49.46 | 6 |
| ATOM | 12490 | CB | ALA | E | 121 | 103.551 | 92.344 | 17.048 | 1.00 | 59.18 | 6 |
| ATOM | 12491 | C | ALA | E | 121 | 101.747 | 90.948 | 16.004 | 1.00 | 48.63 | 6 |
| ATOM | 12492 | O | ALA | E | 121 | 102.121 | 89.973 | 15.343 | 1.00 | 48.09 | 8 |
| ATOM | 12493 | N | ALA | E | 122 | 100.693 | 90.900 | 16.816 | 1.00 | 71.02 | 7 |
| ATOM | 12494 | CA | ALA | E | 122 | 99.915 | 89.676 | 16.974 | 1.00 | 70.10 | 6 |
| ATOM | 12495 | CB | ALA | E | 122 | 98.935 | 89.802 | 18.163 | 1.00 | 13.87 | 6 |
| ATOM | 12496 | C | ALA | E | 122 | 99.169 | 89.333 | 15.690 | 1.00 | 69.94 | 6 |
| ATOM | 12497 | O | ALA | E | 122 | 98.980 | 88.160 | 15.370 | 1.00 | 70.05 | 8 |
| ATOM | 12498 | N | ALA | E | 123 | 98.760 | 90.349 | 14.940 | 1.00 | 178.16 | 7 |
| ATOM | 12499 | CA | ALA | E | 123 | 98.043 | 90.097 | 13.696 | 1.00 | 178.21 | 6 |
| ATOM | 12500 | CB | ALA | E | 123 | 97.028 | 91.221 | 13.429 | 1.00 | 13.87 | 6 |
| ATOM | 12501 | C | ALA | E | 123 | 98.992 | 89.926 | 12.502 | 1.00 | 178.38 | 6 |
| ATOM | 12502 | O | ALA | E | 123 | 98.566 | 90.007 | 11.346 | 1.00 | 179.27 | 8 |
| ATOM | 12503 | N | ALA | E | 124 | 100.273 | 89.683 | 12.785 | 1.00 | 30.70 | 7 |
| ATOM | 12504 | CA | ALA | E | 124 | 101.266 | 89.480 | 11.734 | 1.00 | 29.80 | 6 |
| ATOM | 12505 | CB | ALA | E | 124 | 101.058 | 88.091 | 11.095 | 1.00 | 13.87 | 6 |
| ATOM | 12506 | C | ALA | E | 124 | 101.217 | 90.579 | 10.660 | 1.00 | 30.66 | 6 |
| ATOM | 12507 | O | ALA | E | 124 | 100.847 | 90.329 | 9.513 | 1.00 | 29.87 | 8 |
| ATOM | 12508 | N | ALA | E | 125 | 101.602 | 91.797 | 11.039 | 1.00 | 136.32 | 7 |
| ATOM | 12509 | CA | ALA | E | 125 | 101.581 | 92.914 | 10.098 | 1.00 | 137.92 | 6 |
| ATOM | 12510 | CB | ALA | E | 125 | 100.135 | 93.207 | 9.683 | 1.00 | 64.13 | 6 |
| ATOM | 12511 | C | ALA | E | 125 | 102.248 | 94.210 | 10.581 | 1.00 | 138.86 | 6 |
| ATOM | 12512 | O | ALA | E | 125 | 101.747 | 95.302 | 10.300 | 1.00 | 139.25 | 8 |
| ATOM | 12513 | N | ALA | E | 126 | 103.366 | 94.106 | 11.296 | 1.00 | 62.51 | 7 |
| ATOM | 12514 | CA | ALA | E | 126 | 104.057 | 95.301 | 11.767 | 1.00 | 62.97 | 6 |
| ATOM | 12515 | CB | ALA | E | 126 | 103.524 | 95.716 | 13.123 | 1.00 | 94.98 | 6 |
| ATOM | 12516 | C | ALA | E | 126 | 105.552 | 95.075 | 11.851 | 1.00 | 64.15 | 6 |
| ATOM | 12517 | O | ALA | E | 126 | 106.285 | 95.373 | 10.905 | 1.00 | 64.19 | 8 |
| ATOM | 12518 | N | ALA | E | 127 | 105.992 | 94.550 | 12.993 | 1.00 | 93.46 | 7 |
| ATOM | 12519 | CA | ALA | E | 127 | 107.404 | 94.270 | 13.257 | 1.00 | 94.94 | 6 |
| ATOM | 12520 | CB | ALA | E | 127 | 108.266 | 95.415 | 12.763 | 1.00 | 17.72 | 6 |
| ATOM | 12521 | C | ALA | E | 127 | 107.605 | 94.076 | 14.758 | 1.00 | 96.30 | 6 |
| ATOM | 12522 | O | ALA | E | 127 | 107.633 | 95.046 | 15.523 | 1.00 | 96.29 | 8 |
| ATOM | 12523 | N | ALA | E | 128 | 107.751 | 92.821 | 15.172 | 1.00 | 83.88 | 7 |
| ATOM | 12524 | CA | ALA | E | 128 | 107.920 | 92.495 | 16.584 | 1.00 | 85.18 | 6 |
| ATOM | 12525 | CB | ALA | E | 128 | 107.585 | 91.020 | 16.816 | 1.00 | 113.19 | 6 |
| ATOM | 12526 | C | ALA | E | 128 | 109.308 | 92.813 | 17.146 | 1.00 | 86.19 | 6 |
| ATOM | 12527 | O | ALA | E | 128 | 109.421 | 93.502 | 18.163 | 1.00 | 87.00 | 8 |
| ATOM | 12528 | N | ALA | E | 129 | 110.348 | 92.316 | 16.474 | 1.00 | 69.89 | 7 |
| ATOM | 12529 | CA | ALA | E | 129 | 111.746 | 92.495 | 16.883 | 1.00 | 71.80 | 6 |
| ATOM | 12530 | CB | ALA | E | 129 | 112.022 | 93.936 | 17.341 | 1.00 | 13.87 | 6 |
| ATOM | 12531 | C | ALA | E | 129 | 112.066 | 91.520 | 17.998 | 1.00 | 73.44 | 6 |
| ATOM | 12532 | O | ALA | E | 129 | 111.235 | 90.676 | 18.335 | 1.00 | 73.27 | 8 |
| ATOM | 12533 | N | ALA | E | 130 | 113.266 | 91.642 | 18.558 | 1.00 | 109.86 | 7 |
| ATOM | 12534 | CA | ALA | E | 130 | 113.737 | 90.786 | 19.645 | 1.00 | 112.83 | 6 |
| ATOM | 12535 | CB | ALA | E | 130 | 112.946 | 89.482 | 19.706 | 1.00 | 70.66 | 6 |
| ATOM | 12536 | C | ALA | E | 130 | 115.199 | 90.476 | 19.397 | 1.00 | 115.36 | 6 |
| ATOM | 12537 | O | ALA | E | 130 | 115.932 | 90.093 | 20.312 | 1.00 | 116.61 | 8 |
| ATOM | 12538 | N | ALA | E | 131 | 115.615 | 90.640 | 18.145 | 1.00 | 208.87 | 7 |
| ATOM | 12539 | CA | ALA | E | 131 | 116.989 | 90.379 | 17.751 | 1.00 | 208.87 | 6 |
| ATOM | 12540 | CB | ALA | E | 131 | 117.011 | 89.490 | 16.510 | 1.00 | 139.43 | 6 |
| ATOM | 12541 | C | ALA | E | 131 | 117.724 | 91.692 | 17.485 | 1.00 | 208.87 | 6 |
| ATOM | 12542 | O | ALA | E | 131 | 117.488 | 92.358 | 16.473 | 1.00 | 208.87 | 8 |
| ATOM | 12543 | N | ALA | E | 132 | 118.608 | 92.060 | 18.407 | 1.00 | 160.35 | 7 |
| ATOM | 12544 | CA | ALA | E | 132 | 119.386 | 93.289 | 18.292 | 1.00 | 161.39 | 6 |
| ATOM | 12545 | CB | ALA | E | 132 | 120.470 | 93.311 | 19.364 | 1.00 | 126.25 | 6 |
| ATOM | 12546 | C | ALA | E | 132 | 120.012 | 93.407 | 16.905 | 1.00 | 162.45 | 6 |
| ATOM | 12547 | O | ALA | E | 132 | 120.064 | 92.430 | 16.159 | 1.00 | 162.81 | 8 |
| ATOM | 12548 | N | ALA | E | 133 | 120.489 | 94.600 | 16.557 | 1.00 | 148.54 | 7 |
| ATOM | 12549 | CA | ALA | E | 133 | 121.100 | 94.798 | 15.244 | 1.00 | 149.35 | 6 |
| ATOM | 12550 | CB | ALA | E | 133 | 120.035 | 94.653 | 14.159 | 1.00 | 137.78 | 6 |
| ATOM | 12551 | C | ALA | E | 133 | 121.850 | 96.122 | 15.071 | 1.00 | 149.95 | 6 |
| ATOM | 12552 | O | ALA | E | 133 | 122.803 | 96.409 | 15.796 | 1.00 | 150.94 | 8 |
| ATOM | 12553 | N | ALA | E | 134 | 121.411 | 96.919 | 14.099 | 1.00 | 75.98 | 7 |
| ATOM | 12554 | CA | ALA | E | 134 | 122.029 | 98.205 | 13.794 | 1.00 | 76.04 | 6 |
| ATOM | 12555 | CB | ALA | E | 134 | 122.453 | 98.229 | 12.326 | 1.00 | 130.28 | 6 |
| ATOM | 12556 | C | ALA | E | 134 | 121.152 | 99.422 | 14.099 | 1.00 | 76.45 | 6 |
| ATOM | 12557 | O | ALA | E | 134 | 120.927 | 99.759 | 15.265 | 1.00 | 75.74 | 8 |
| ATOM | 12558 | N | ALA | E | 135 | 120.663 | 100.079 | 13.048 | 1.00 | 136.30 | 7 |
| ATOM | 12559 | CA | ALA | E | 135 | 119.827 | 101.266 | 13.211 | 1.00 | 137.55 | 6 |
| ATOM | 12560 | CB | ALA | E | 135 | 120.504 | 102.246 | 14.141 | 1.00 | 90.41 | 6 |
| ATOM | 12561 | C | ALA | E | 135 | 119.547 | 101.947 | 11.877 | 1.00 | 138.32 | 6 |
| ATOM | 12562 | O | ALA | E | 135 | 120.384 | 101.929 | 10.973 | 1.00 | 138.96 | 8 |
| ATOM | 12563 | N | ALA | E | 136 | 118.370 | 102.561 | 11.773 | 1.00 | 190.44 | 7 |
| ATOM | 12564 | CA | ALA | E | 136 | 117.953 | 103.260 | 10.561 | 1.00 | 191.59 | 6 |
| ATOM | 12565 | CB | ALA | E | 136 | 116.494 | 102.945 | 10.255 | 1.00 | 140.21 | 6 |
| ATOM | 12566 | C | ALA | E | 136 | 118.140 | 104.768 | 10.690 | 1.00 | 192.38 | 6 |
| ATOM | 12567 | O | ALA | E | 136 | 117.752 | 105.528 | 9.799 | 1.00 | 193.07 | 8 |
| ATOM | 12568 | N | ALA | E | 137 | 118.728 | 105.189 | 11.808 | 1.00 | 207.72 | 7 |
| ATOM | 12569 | CA | ALA | E | 137 | 118.989 | 106.602 | 12.081 | 1.00 | 207.46 | 6 |
| ATOM | 12570 | CB | ALA | E | 137 | 117.691 | 107.408 | 12.028 | 1.00 | 117.55 | 6 |
| ATOM | 12571 | C | ALA | E | 137 | 119.656 | 106.785 | 13.444 | 1.00 | 207.69 | 6 |
| ATOM | 12572 | O | ALA | E | 137 | 120.737 | 107.364 | 13.542 | 1.00 | 208.14 | 8 |
| ATOM | 12573 | N | ALA | E | 138 | 119.006 | 106.291 | 14.492 | 1.00 | 150.77 | 7 |
| ATOM | 12574 | CA | ALA | E | 138 | 119.539 | 106.406 | 15.842 | 1.00 | 150.79 | 6 |
| ATOM | 12575 | CB | ALA | E | 138 | 118.421 | 106.738 | 16.809 | 1.00 | 132.19 | 6 |
| ATOM | 12576 | C | ALA | E | 138 | 120.241 | 105.125 | 16.270 | 1.00 | 150.96 | 6 |
| ATOM | 12577 | O | ALA | E | 138 | 121.458 | 105.009 | 16.150 | 1.00 | 151.30 | 8 |
| ATOM | 12578 | N | ALA | E | 139 | 119.473 | 104.167 | 16.776 | 1.00 | 113.98 | 7 |
| ATOM | 12579 | CA | ALA | E | 139 | 120.033 | 102.895 | 17.212 | 1.00 | 113.98 | 6 |
| ATOM | 12580 | CB | ALA | E | 139 | 120.454 | 102.982 | 18.681 | 1.00 | 93.02 | 6 |
| ATOM | 12581 | C | ALA | E | 139 | 119.007 | 101.778 | 17.012 | 1.00 | 114.55 | 6 |
| ATOM | 12582 | O | ALA | E | 139 | 118.321 | 101.733 | 15.987 | 1.00 | 114.68 | 8 |
| ATOM | 12583 | N | ALA | E | 140 | 118.912 | 100.877 | 17.990 | 1.00 | 208.87 | 7 |
| ATOM | 12584 | CA | ALA | E | 140 | 117.963 | 99.763 | 17.944 | 1.00 | 208.87 | 6 |
| ATOM | 12585 | CB | ALA | E | 140 | 118.526 | 98.625 | 17.107 | 1.00 | 79.54 | 6 |
| ATOM | 12586 | C | ALA | E | 140 | 117.646 | 99.268 | 19.355 | 1.00 | 208.87 | 6 |
| ATOM | 12587 | O | ALA | E | 140 | 118.520 | 99.234 | 20.221 | 1.00 | 208.87 | 8 |
| ATOM | 12588 | N | ALA | E | 141 | 116.395 | 98.878 | 19.580 | 1.00 | 83.09 | 7 |
| ATOM | 12589 | CA | ALA | E | 141 | 115.965 | 98.391 | 20.888 | 1.00 | 83.09 | 6 |
| ATOM | 12590 | CB | ALA | E | 141 | 115.448 | 99.555 | 21.730 | 1.00 | 160.18 | 6 |
| ATOM | 12591 | C | ALA | E | 141 | 114.884 | 97.310 | 20.768 | 1.00 | 83.22 | 6 |
| ATOM | 12592 | O | ALA | E | 141 | 114.196 | 97.215 | 19.751 | 1.00 | 82.93 | 8 |
| ATOM | 12593 | N | ALA | E | 142 | 114.731 | 96.503 | 21.814 | 1.00 | 194.90 | 7 |
| ATOM | 12594 | CA | ALA | E | 142 | 113.736 | 95.433 | 21.814 | 1.00 | 194.98 | 6 |
| ATOM | 12595 | CB | ALA | E | 142 | 113.997 | 94.476 | 22.968 | 1.00 | 131.90 | 6 |
| ATOM | 12596 | C | ALA | E | 142 | 112.318 | 95.983 | 21.900 | 1.00 | 194.65 | 6 |
| ATOM | 12597 | O | ALA | E | 142 | 111.654 | 95.855 | 22.931 | 1.00 | 195.16 | 8 |
| ATOM | 12598 | N | ALA | E | 143 | 111.863 | 96.590 | 20.806 | 1.00 | 136.97 | 7 |
| ATOM | 12599 | CA | ALA | E | 143 | 110.523 | 97.170 | 20.724 | 1.00 | 136.69 | 6 |
| ATOM | 12600 | CB | ALA | E | 143 | 110.267 | 98.061 | 21.932 | 1.00 | 183.63 | 6 |
| ATOM | 12601 | C | ALA | E | 143 | 110.322 | 97.972 | 19.437 | 1.00 | 136.26 | 6 |
| ATOM | 12602 | O | ALA | E | 143 | 109.233 | 97.972 | 18.866 | 1.00 | 136.76 | 8 |
| ATOM | 12603 | N | ALA | E | 144 | 111.377 | 98.661 | 19.003 | 1.00 | 208.87 | 7 |
| ATOM | 12604 | CA | ALA | E | 144 | 111.346 | 99.474 | 17.787 | 1.00 | 208.87 | 6 |
| ATOM | 12605 | CB | ALA | E | 144 | 112.786 | 99.817 | 17.342 | 1.00 | 78.71 | 6 |
| ATOM | 12606 | C | ALA | E | 144 | 110.604 | 98.719 | 16.681 | 1.00 | 208.87 | 6 |
| ATOM | 12607 | O | ALA | E | 144 | 111.203 | 97.942 | 15.937 | 1.00 | 208.87 | 8 |
| ATOM | 12608 | N | ALA | E | 145 | 109.295 | 98.955 | 16.589 | 1.00 | 138.23 | 7 |
| ATOM | 12609 | CA | ALA | E | 145 | 108.446 | 98.296 | 15.598 | 1.00 | 136.15 | 6 |
| ATOM | 12610 | CB | ALA | E | 145 | 107.014 | 98.202 | 16.127 | 1.00 | 141.89 | 6 |
| ATOM | 12611 | C | ALA | E | 145 | 108.465 | 98.989 | 14.229 | 1.00 | 134.71 | 6 |
| ATOM | 12612 | O | ALA | E | 145 | 107.579 | 99.783 | 13.903 | 1.00 | 134.55 | 8 |
| ATOM | 12613 | N | ALA | E | 146 | 109.481 | 98.672 | 13.432 | 1.00 | 160.11 | 7 |
| ATOM | 12614 | CA | ALA | E | 146 | 109.628 | 99.241 | 12.099 | 1.00 | 157.72 | 6 |
| ATOM | 12615 | CB | ALA | E | 146 | 110.936 | 98.766 | 11.484 | 1.00 | 56.13 | 6 |
| ATOM | 12616 | C | ALA | E | 146 | 108.446 | 98.808 | 11.236 | 1.00 | 156.32 | 6 |
| ATOM | 12617 | O | ALA | E | 146 | 107.686 | 97.928 | 11.628 | 1.00 | 156.69 | 8 |
| ATOM | 12618 | N | ALA | E | 147 | 108.289 | 99.422 | 10.066 | 1.00 | 176.44 | 7 |
| ATOM | 12619 | CA | ALA | E | 147 | 107.185 | 99.075 | 9.173 | 1.00 | 174.35 | 6 |
| ATOM | 12620 | CB | ALA | E | 147 | 106.884 | 100.234 | 8.225 | 1.00 | 13.87 | 6 |
| ATOM | 12621 | C | ALA | E | 147 | 107.479 | 97.810 | 8.370 | 1.00 | 172.71 | 6 |
| ATOM | 12622 | O | ALA | E | 147 | 108.565 | 97.237 | 8.473 | 1.00 | 173.54 | 8 |
| ATOM | 12623 | N | ALA | E | 148 | 106.501 | 97.383 | 7.573 | 1.00 | 146.02 | 7 |
| ATOM | 12624 | CA | ALA | E | 148 | 106.631 | 96.188 | 6.743 | 1.00 | 143.18 | 6 |
| ATOM | 12625 | CB | ALA | E | 148 | 105.415 | 96.067 | 5.803 | 1.00 | 63.85 | 6 |
| ATOM | 12626 | C | ALA | E | 148 | 107.923 | 96.224 | 5.929 | 1.00 | 141.42 | 6 |
| ATOM | 12627 | O | ALA | E | 148 | 108.558 | 95.193 | 5.703 | 1.00 | 141.69 | 8 |
| ATOM | 12628 | N | ALA | E | 149 | 108.306 | 97.424 | 5.505 | 1.00 | 90.34 | 7 |
| ATOM | 12629 | CA | ALA | E | 149 | 109.508 | 97.626 | 4.706 | 1.00 | 87.62 | 6 |
| ATOM | 12630 | CB | ALA | E | 149 | 109.302 | 98.810 | 3.763 | 1.00 | 63.96 | 6 |
| ATOM | 12631 | C | ALA | E | 149 | 110.759 | 97.844 | 5.554 | 1.00 | 85.77 | 6 |
| ATOM | 12632 | O | ALA | E | 149 | 111.758 | 97.152 | 5.367 | 1.00 | 85.86 | 8 |
| ATOM | 12633 | N | ALA | E | 150 | 110.692 | 98.801 | 6.483 | 1.00 | 51.33 | 7 |
| ATOM | 12634 | CA | ALA | E | 150 | 111.815 | 99.139 | 7.363 | 1.00 | 49.33 | 6 |
| ATOM | 12635 | CB | ALA | E | 150 | 111.346 | 100.029 | 8.528 | 1.00 | 38.77 | 6 |
| ATOM | 12636 | C | ALA | E | 150 | 112.512 | 97.893 | 7.891 | 1.00 | 48.52 | 6 |
| ATOM | 12637 | O | ALA | E | 150 | 113.613 | 97.965 | 8.443 | 1.00 | 48.14 | 8 |
| ATOM | 12638 | N | ALA | E | 151 | 111.860 | 96.749 | 7.733 | 1.00 | 73.27 | 7 |
| ATOM | 12639 | CA | ALA | E | 151 | 112.454 | 95.495 | 8.140 | 1.00 | 72.54 | 6 |
| ATOM | 12640 | CB | ALA | E | 151 | 111.391 | 94.444 | 8.316 | 1.00 | 39.74 | 6 |
| ATOM | 12641 | C | ALA | E | 151 | 113.359 | 95.135 | 6.972 | 1.00 | 72.88 | 6 |
| ATOM | 12642 | O | ALA | E | 151 | 114.579 | 95.094 | 7.110 | 1.00 | 73.09 | 8 |
| ATOM | 12643 | N | ALA | E | 152 | 112.747 | 94.897 | 5.814 | 1.00 | 99.11 | 7 |
| ATOM | 12644 | CA | ALA | E | 152 | 113.487 | 94.550 | 4.606 | 1.00 | 100.05 | 6 |
| ATOM | 12645 | CB | ALA | E | 152 | 112.624 | 94.792 | 3.379 | 1.00 | 130.30 | 6 |
| ATOM | 12646 | C | ALA | E | 152 | 114.745 | 95.397 | 4.532 | 1.00 | 100.26 | 6 |
| ATOM | 12647 | O | ALA | E | 152 | 115.823 | 94.909 | 4.196 | 1.00 | 100.22 | 8 |
| ATOM | 12648 | N | ALA | E | 153 | 114.591 | 96.677 | 4.848 | 1.00 | 88.91 | 7 |
| ATOM | 12649 | CA | ALA | E | 153 | 115.710 | 97.599 | 4.838 | 1.00 | 88.85 | 6 |
| ATOM | 12650 | CB | ALA | E | 153 | 115.309 | 98.931 | 5.469 | 1.00 | 53.58 | 6 |
| ATOM | 12651 | C | ALA | E | 153 | 116.814 | 96.950 | 5.646 | 1.00 | 89.26 | 6 |
| ATOM | 12652 | O | ALA | E | 153 | 117.895 | 96.680 | 5.130 | 1.00 | 90.15 | 8 |
| ATOM | 12653 | N | ALA | E | 154 | 116.520 | 96.679 | 6.913 | 1.00 | 65.53 | 7 |
| ATOM | 12654 | CA | ALA | E | 154 | 117.479 | 96.058 | 7.816 | 1.00 | 64.96 | 6 |
| ATOM | 12655 | CB | ALA | E | 154 | 116.908 | 96.042 | 9.224 | 1.00 | 20.30 | 6 |
| ATOM | 12656 | C | ALA | E | 154 | 117.873 | 94.640 | 7.389 | 1.00 | 64.65 | 6 |
| ATOM | 12657 | O | ALA | E | 154 | 118.616 | 93.956 | 8.097 | 1.00 | 64.49 | 8 |
| ATOM | 12658 | N | ALA | E | 155 | 117.381 | 94.211 | 6.228 | 1.00 | 100.32 | 7 |
| ATOM | 12659 | CA | ALA | E | 155 | 117.680 | 92.880 | 5.701 | 1.00 | 101.00 | 6 |
| ATOM | 12660 | CB | ALA | E | 155 | 116.391 | 92.209 | 5.219 | 1.00 | 79.53 | 6 |
| ATOM | 12661 | C | ALA | E | 155 | 118.713 | 92.917 | 4.564 | 1.00 | 101.26 | 6 |
| ATOM | 12662 | O | ALA | E | 155 | 119.751 | 92.228 | 4.690 | 1.00 | 101.36 | 8 |
| ATOM | 12663 | OXT | ALA | E | 155 | 118.478 | 93.623 | 3.557 | 1.00 | 79.48 | 8 |
| ATOM | 12664 | CB | ALA | F | 452 | 120.550 | 88.822 | 12.955 | 1.00 | 38.60 | 6 |
| ATOM | 12665 | C | ALA | F | 452 | 119.171 | 90.755 | 12.218 | 1.00 | 67.11 | 6 |
| ATOM | 12666 | O | ALA | F | 452 | 119.148 | 91.619 | 13.095 | 1.00 | 67.68 | 8 |
| ATOM | 12667 | N | ALA | F | 452 | 121.671 | 90.756 | 11.907 | 1.00 | 66.01 | 7 |
| ATOM | 12668 | CA | ALA | F | 452 | 120.425 | 89.931 | 11.929 | 1.00 | 66.79 | 6 |
| ATOM | 12669 | N | ALA | F | 453 | 118.113 | 90.454 | 11.482 | 1.00 | 116.35 | 7 |
| ATOM | 12670 | CA | ALA | F | 453 | 116.858 | 91.167 | 11.631 | 1.00 | 116.97 | 6 |
| ATOM | 12671 | CB | ALA | F | 453 | 115.972 | 90.902 | 10.409 | 1.00 | 200.00 | 6 |
| ATOM | 12672 | C | ALA | F | 453 | 116.097 | 90.834 | 12.915 | 1.00 | 116.82 | 6 |
| ATOM | 12673 | O | ALA | F | 453 | 116.690 | 90.551 | 13.957 | 1.00 | 116.02 | 8 |
| ATOM | 12674 | N | ALA | F | 454 | 114.771 | 90.874 | 12.808 | 1.00 | 83.92 | 7 |
| ATOM | 12675 | CA | ALA | F | 454 | 113.864 | 90.613 | 13.914 | 1.00 | 83.90 | 6 |
| ATOM | 12676 | CB | ALA | F | 454 | 113.159 | 91.891 | 14.286 | 1.00 | 74.11 | 6 |
| ATOM | 12677 | C | ALA | F | 454 | 112.836 | 89.557 | 13.534 | 1.00 | 83.87 | 6 |
| ATOM | 12678 | O | ALA | F | 454 | 113.056 | 88.756 | 12.626 | 1.00 | 83.95 | 8 |
| ATOM | 12679 | N | ALA | F | 455 | 111.700 | 89.579 | 14.225 | 1.00 | 124.76 | 7 |
| ATOM | 12680 | CA | ALA | F | 455 | 110.627 | 88.625 | 13.974 | 1.00 | 125.32 | 6 |
| ATOM | 12681 | CB | ALA | F | 455 | 110.726 | 87.472 | 14.964 | 1.00 | 36.96 | 6 |
| ATOM | 12682 | C | ALA | F | 455 | 109.250 | 89.277 | 14.075 | 1.00 | 125.72 | 6 |
| ATOM | 12683 | O | ALA | F | 455 | 109.130 | 90.458 | 14.403 | 1.00 | 126.19 | 8 |
| ATOM | 12684 | N | MET | F | 456 | 108.217 | 88.492 | 13.785 | 1.00 | 125.26 | 7 |
| ATOM | 12685 | CA | MET | F | 456 | 106.837 | 88.960 | 13.855 | 1.00 | 125.09 | 6 |
| ATOM | 12686 | CB | MET | F | 456 | 106.418 | 89.616 | 12.540 | 1.00 | 119.07 | 6 |
| ATOM | 12687 | CG | MET | F | 456 | 105.019 | 90.211 | 12.575 | 1.00 | 121.00 | 6 |
| ATOM | 12688 | SD | MET | F | 456 | 104.271 | 90.298 | 10.947 | 1.00 | 122.21 | 16 |
| ATOM | 12689 | CE | MET | F | 456 | 104.846 | 91.866 | 10.393 | 1.00 | 123.98 | 6 |
| ATOM | 12690 | C | MET | F | 456 | 105.905 | 87.785 | 14.144 | 1.00 | 124.57 | 6 |
| ATOM | 12691 | O | MET | F | 456 | 106.307 | 86.620 | 14.043 | 1.00 | 124.83 | 8 |
| ATOM | 12692 | N | GLY | F | 457 | 104.662 | 88.098 | 14.500 | 1.00 | 146.13 | 7 |
| ATOM | 12693 | CA | GLY | F | 457 | 103.687 | 87.065 | 14.795 | 1.00 | 144.47 | 6 |
| ATOM | 12694 | C | GLY | F | 457 | 103.554 | 86.800 | 16.280 | 1.00 | 143.55 | 6 |
| ATOM | 12695 | O | GLY | F | 457 | 104.230 | 87.428 | 17.095 | 1.00 | 143.63 | 8 |
| ATOM | 12696 | N | ALA | F | 458 | 102.678 | 85.863 | 16.630 | 1.00 | 89.49 | 7 |
| ATOM | 12697 | CA | ALA | F | 458 | 102.445 | 85.502 | 18.025 | 1.00 | 87.39 | 6 |
| ATOM | 12698 | CB | ALA | F | 458 | 101.226 | 84.599 | 18.130 | 1.00 | 117.14 | 6 |
| ATOM | 12699 | C | ALA | F | 458 | 103.663 | 84.804 | 18.604 | 1.00 | 85.65 | 6 |
| ATOM | 12700 | O | ALA | F | 458 | 103.966 | 84.944 | 19.783 | 1.00 | 85.39 | 8 |
| ATOM | 12701 | N | GLU | F | 459 | 104.358 | 84.049 | 17.764 | 1.00 | 58.03 | 7 |
| ATOM | 12702 | CA | GLU | F | 459 | 105.549 | 83.341 | 18.193 | 1.00 | 56.94 | 6 |
| ATOM | 12703 | CB | GLU | F | 459 | 106.075 | 82.447 | 17.064 | 1.00 | 92.28 | 6 |
| ATOM | 12704 | CG | GLU | F | 459 | 107.264 | 81.581 | 17.463 | 1.00 | 92.76 | 6 |
| ATOM | 12705 | CD | GLU | F | 459 | 107.660 | 80.595 | 16.383 | 1.00 | 93.55 | 6 |
| ATOM | 12706 | OE1 | GLU | F | 459 | 108.028 | 81.041 | 15.276 | 1.00 | 93.66 | 8 |
| ATOM | 12707 | OE2 | GLU | F | 459 | 107.603 | 79.372 | 16.640 | 1.00 | 94.23 | 8 |
| ATOM | 12708 | C | GLU | F | 459 | 106.610 | 84.359 | 18.587 | 1.00 | 55.62 | 6 |
| ATOM | 12709 | O | GLU | F | 459 | 107.421 | 84.111 | 19.480 | 1.00 | 55.42 | 8 |
| ATOM | 12710 | N | ALA | F | 460 | 106.599 | 85.505 | 17.913 | 1.00 | 57.59 | 7 |
| ATOM | 12711 | CA | ALA | F | 460 | 107.554 | 86.573 | 18.199 | 1.00 | 56.41 | 6 |
| ATOM | 12712 | CB | ALA | F | 460 | 107.422 | 87.680 | 17.168 | 1.00 | 66.61 | 6 |
| ATOM | 12713 | C | ALA | F | 460 | 107.262 | 87.122 | 19.589 | 1.00 | 56.10 | 6 |
| ATOM | 12714 | O | ALA | F | 460 | 108.147 | 87.194 | 20.449 | 1.00 | 54.89 | 8 |
| ATOM | 12715 | N | ILE | F | 461 | 106.006 | 87.513 | 19.792 | 1.00 | 86.97 | 7 |
| ATOM | 12716 | CA | ILE | F | 461 | 105.564 | 88.042 | 21.071 | 1.00 | 86.53 | 6 |
| ATOM | 12717 | CB | ILE | F | 461 | 104.083 | 88.371 | 21.057 | 1.00 | 62.08 | 6 |
| ATOM | 12718 | CG2 | ILE | F | 461 | 103.727 | 89.159 | 22.294 | 1.00 | 61.62 | 6 |
| ATOM | 12719 | CG1 | ILE | F | 461 | 103.744 | 89.154 | 19.786 | 1.00 | 61.89 | 6 |
| ATOM | 12720 | CD1 | ILE | F | 461 | 104.631 | 90.351 | 19.528 | 1.00 | 61.14 | 6 |
| ATOM | 12721 | C | ILE | F | 461 | 105.795 | 86.945 | 22.075 | 1.00 | 87.34 | 6 |
| ATOM | 12722 | O | ILE | F | 461 | 106.306 | 87.186 | 23.160 | 1.00 | 88.20 | 8 |
| ATOM | 12723 | N | GLN | F | 462 | 105.410 | 85.731 | 21.703 | 1.00 | 59.54 | 7 |
| ATOM | 12724 | CA | GLN | F | 462 | 105.620 | 84.584 | 22.572 | 1.00 | 59.73 | 6 |
| ATOM | 12725 | CB | GLN | F | 462 | 105.355 | 83.280 | 21.818 | 1.00 | 89.81 | 6 |
| ATOM | 12726 | CG | GLN | F | 462 | 105.526 | 82.037 | 22.668 | 1.00 | 90.71 | 6 |
| ATOM | 12727 | CD | GLN | F | 462 | 105.328 | 80.761 | 21.878 | 1.00 | 91.74 | 6 |
| ATOM | 12728 | OE1 | GLN | F | 462 | 106.032 | 80.516 | 20.903 | 1.00 | 92.84 | 8 |
| ATOM | 12729 | NE2 | GLN | F | 462 | 104.371 | 79.938 | 22.297 | 1.00 | 92.15 | 7 |
| ATOM | 12730 | C | GLN | F | 462 | 107.088 | 84.683 | 22.936 | 1.00 | 59.95 | 6 |
| ATOM | 12731 | O | GLN | F | 462 | 107.445 | 84.850 | 24.106 | 1.00 | 59.75 | 8 |
| ATOM | 12732 | N | GLU | F | 463 | 107.927 | 84.607 | 21.907 | 1.00 | 102.73 | 7 |
| ATOM | 12733 | CA | GLU | F | 463 | 109.368 | 84.713 | 22.065 | 1.00 | 103.40 | 6 |
| ATOM | 12734 | CB | GLU | F | 463 | 110.006 | 85.097 | 20.725 | 1.00 | 141.27 | 6 |
| ATOM | 12735 | CG | GLU | F | 463 | 111.504 | 85.335 | 20.773 | 1.00 | 141.64 | 6 |
| ATOM | 12736 | CD | GLU | F | 463 | 112.059 | 85.810 | 19.441 | 1.00 | 141.81 | 6 |
| ATOM | 12737 | OE1 | GLU | F | 463 | 111.570 | 86.838 | 18.924 | 1.00 | 141.52 | 8 |
| ATOM | 12738 | OE2 | GLU | F | 463 | 112.985 | 85.156 | 18.913 | 1.00 | 142.23 | 8 |
| ATOM | 12739 | C | GLU | F | 463 | 109.635 | 85.788 | 23.115 | 1.00 | 104.05 | 6 |
| ATOM | 12740 | O | GLU | F | 463 | 110.198 | 85.511 | 24.176 | 1.00 | 103.97 | 8 |
| ATOM | 12741 | N | LEU | F | 464 | 109.205 | 87.011 | 22.828 | 1.00 | 65.20 | 7 |
| ATOM | 12742 | CA | LEU | F | 464 | 109.396 | 88.108 | 23.763 | 1.00 | 65.47 | 6 |
| ATOM | 12743 | CB | LEU | F | 464 | 108.599 | 89.335 | 23.302 | 1.00 | 23.98 | 6 |
| ATOM | 12744 | CG | LEU | F | 464 | 109.111 | 90.041 | 22.045 | 1.00 | 22.38 | 6 |
| ATOM | 12745 | CD1 | LEU | F | 464 | 108.170 | 91.160 | 21.594 | 1.00 | 21.31 | 6 |
| ATOM | 12746 | CD2 | LEU | F | 464 | 110.481 | 90.596 | 22.357 | 1.00 | 22.00 | 6 |
| ATOM | 12747 | C | LEU | F | 464 | 108.967 | 87.695 | 25.173 | 1.00 | 66.98 | 6 |
| ATOM | 12748 | O | LEU | F | 464 | 109.739 | 87.815 | 26.124 | 1.00 | 66.60 | 8 |
| ATOM | 12749 | N | LEU | F | 465 | 107.739 | 87.198 | 25.304 | 1.00 | 90.78 | 7 |
| ATOM | 12750 | CA | LEU | F | 465 | 107.235 | 86.784 | 26.607 | 1.00 | 93.27 | 6 |
| ATOM | 12751 | CB | LEU | F | 465 | 105.764 | 86.351 | 26.519 | 1.00 | 57.35 | 6 |
| ATOM | 12752 | CG | LEU | F | 465 | 104.745 | 87.501 | 26.623 | 1.00 | 56.24 | 6 |
| ATOM | 12753 | CD1 | LEU | F | 465 | 104.604 | 88.226 | 25.298 | 1.00 | 55.61 | 6 |
| ATOM | 12754 | CD2 | LEU | F | 465 | 103.401 | 86.955 | 27.048 | 1.00 | 55.69 | 6 |
| ATOM | 12755 | C | LEU | F | 465 | 108.085 | 85.669 | 27.191 | 1.00 | 95.50 | 6 |
| ATOM | 12756 | O | LEU | F | 465 | 108.746 | 85.857 | 28.212 | 1.00 | 96.05 | 8 |
| ATOM | 12757 | N | ALA | F | 466 | 108.080 | 84.510 | 26.547 | 1.00 | 162.20 | 7 |
| ATOM | 12758 | CA | ALA | F | 466 | 108.877 | 83.396 | 27.033 | 1.00 | 165.26 | 6 |
| ATOM | 12759 | CB | ALA | F | 466 | 108.807 | 82.225 | 26.057 | 1.00 | 140.53 | 6 |
| ATOM | 12760 | C | ALA | F | 466 | 110.313 | 83.873 | 27.174 | 1.00 | 167.47 | 6 |
| ATOM | 12761 | O | ALA | F | 466 | 110.677 | 84.918 | 26.638 | 1.00 | 167.66 | 8 |
| ATOM | 12762 | N | ALA | F | 467 | 111.110 | 83.106 | 27.912 | 1.00 | 208.87 | 7 |
| ATOM | 12763 | CA | ALA | F | 467 | 112.522 | 83.402 | 28.143 | 1.00 | 208.87 | 6 |
| ATOM | 12764 | CB | ALA | F | 467 | 113.347 | 82.899 | 26.965 | 1.00 | 176.73 | 6 |
| ATOM | 12765 | C | ALA | F | 467 | 112.864 | 84.867 | 28.428 | 1.00 | 208.87 | 6 |
| ATOM | 12766 | O | ALA | F | 467 | 113.292 | 85.198 | 29.533 | 1.00 | 208.87 | 8 |
| ATOM | 12767 | N | ALA | F | 468 | 112.687 | 85.728 | 27.428 | 1.00 | 164.03 | 7 |
| ATOM | 12768 | CA | ALA | F | 468 | 112.986 | 87.156 | 27.541 | 1.00 | 165.22 | 6 |
| ATOM | 12769 | CB | ALA | F | 468 | 112.349 | 87.912 | 26.384 | 1.00 | 105.32 | 6 |
| ATOM | 12770 | C | ALA | F | 468 | 112.557 | 87.778 | 28.864 | 1.00 | 166.15 | 6 |
| ATOM | 12771 | O | ALA | F | 468 | 113.163 | 87.515 | 29.904 | 1.00 | 167.06 | 8 |
| ATOM | 12772 | N | ASP | F | 469 | 111.518 | 88.609 | 28.827 | 1.00 | 109.62 | 7 |
| ATOM | 12773 | CA | ASP | F | 469 | 111.046 | 89.258 | 30.047 | 1.00 | 109.91 | 6 |
| ATOM | 12774 | CB | ASP | F | 469 | 109.794 | 90.103 | 29.773 | 1.00 | 192.11 | 6 |
| ATOM | 12775 | CG | ASP | F | 469 | 108.508 | 89.325 | 29.954 | 1.00 | 193.81 | 6 |
| ATOM | 12776 | OD1 | ASP | F | 469 | 108.331 | 88.294 | 29.274 | 1.00 | 195.21 | 8 |
| ATOM | 12777 | OD2 | ASP | F | 469 | 107.671 | 89.751 | 30.779 | 1.00 | 195.31 | 8 |
| ATOM | 12778 | C | ASP | F | 469 | 110.747 | 88.180 | 31.078 | 1.00 | 108.88 | 6 |
| ATOM | 12779 | O | ASP | F | 469 | 110.533 | 88.464 | 32.256 | 1.00 | 108.46 | 8 |
| ATOM | 12780 | N | ALA | F | 470 | 110.751 | 86.936 | 30.611 | 1.00 | 123.41 | 7 |
| ATOM | 12781 | CA | ALA | F | 470 | 110.504 | 85.794 | 31.465 | 1.00 | 123.29 | 6 |
| ATOM | 12782 | CB | ALA | F | 470 | 110.787 | 84.519 | 30.715 | 1.00 | 39.27 | 6 |
| ATOM | 12783 | C | ALA | F | 470 | 111.398 | 85.904 | 32.692 | 1.00 | 123.52 | 6 |
| ATOM | 12784 | O | ALA | F | 470 | 111.131 | 85.282 | 33.720 | 1.00 | 123.75 | 8 |
| ATOM | 12785 | N | GLU | F | 471 | 112.470 | 86.684 | 32.581 | 1.00 | 100.50 | 7 |
| ATOM | 12786 | CA | GLU | F | 471 | 113.344 | 86.887 | 33.724 | 1.00 | 101.02 | 6 |
| ATOM | 12787 | CB | GLU | F | 471 | 114.746 | 87.314 | 33.277 | 1.00 | 121.34 | 6 |
| ATOM | 12788 | CG | GLU | F | 471 | 114.801 | 88.468 | 32.298 | 1.00 | 121.94 | 6 |
| ATOM | 12789 | CD | GLU | F | 471 | 116.230 | 88.814 | 31.909 | 1.00 | 123.23 | 6 |
| ATOM | 12790 | OE1 | GLU | F | 471 | 116.939 | 87.934 | 31.375 | 1.00 | 124.22 | 8 |
| ATOM | 12791 | OE2 | GLU | F | 471 | 116.649 | 89.966 | 32.142 | 1.00 | 124.59 | 8 |
| ATOM | 12792 | C | GLU | F | 471 | 112.649 | 87.989 | 34.508 | 1.00 | 101.49 | 6 |
| ATOM | 12793 | O | GLU | F | 471 | 113.089 | 89.136 | 34.534 | 1.00 | 102.02 | 8 |
| ATOM | 12794 | N | ALA | F | 472 | 111.535 | 87.610 | 35.130 | 1.00 | 134.27 | 7 |
| ATOM | 12795 | CA | ALA | F | 472 | 110.694 | 88.518 | 35.901 | 1.00 | 134.15 | 6 |
| ATOM | 12796 | CB | ALA | F | 472 | 109.315 | 87.893 | 36.108 | 1.00 | 153.46 | 6 |
| ATOM | 12797 | C | ALA | F | 472 | 111.272 | 88.942 | 37.237 | 1.00 | 133.41 | 6 |
| ATOM | 12798 | O | ALA | F | 472 | 111.185 | 90.112 | 37.602 | 1.00 | 133.06 | 8 |
| ATOM | 12799 | N | LEU | F | 473 | 111.843 | 87.999 | 37.978 | 1.00 | 152.73 | 7 |
| ATOM | 12800 | CA | LEU | F | 473 | 112.428 | 88.331 | 39.269 | 1.00 | 153.61 | 6 |
| ATOM | 12801 | CB | LEU | F | 473 | 113.378 | 87.226 | 39.731 | 1.00 | 199.24 | 6 |
| ATOM | 12802 | CG | LEU | F | 473 | 112.750 | 85.898 | 40.151 | 1.00 | 200.51 | 6 |
| ATOM | 12803 | CD1 | LEU | F | 473 | 113.853 | 84.921 | 40.529 | 1.00 | 200.81 | 6 |
| ATOM | 12804 | CD2 | LEU | F | 473 | 111.804 | 86.123 | 41.325 | 1.00 | 200.52 | 6 |
| ATOM | 12805 | C | LEU | F | 473 | 113.197 | 89.637 | 39.142 | 1.00 | 153.51 | 6 |
| ATOM | 12806 | O | LEU | F | 473 | 113.320 | 90.396 | 40.105 | 1.00 | 154.38 | 8 |
| ATOM | 12807 | N | GLU | F | 474 | 113.710 | 89.890 | 37.941 | 1.00 | 107.94 | 7 |
| ATOM | 12808 | CA | GLU | F | 474 | 114.464 | 91.104 | 37.672 | 1.00 | 107.24 | 6 |
| ATOM | 12809 | CB | GLU | F | 474 | 115.330 | 90.911 | 36.420 | 1.00 | 140.51 | 6 |
| ATOM | 12810 | CG | GLU | F | 474 | 116.474 | 89.913 | 36.651 | 1.00 | 140.87 | 6 |
| ATOM | 12811 | CD | GLU | F | 474 | 117.262 | 89.577 | 35.394 | 1.00 | 141.43 | 6 |
| ATOM | 12812 | OE1 | GLU | F | 474 | 117.742 | 90.509 | 34.717 | 1.00 | 142.23 | 8 |
| ATOM | 12813 | OE2 | GLU | F | 474 | 117.413 | 88.373 | 35.089 | 1.00 | 141.78 | 8 |
| ATOM | 12814 | C | GLU | F | 474 | 113.517 | 92.292 | 37.526 | 1.00 | 107.50 | 6 |
| ATOM | 12815 | O | GLU | F | 474 | 113.692 | 93.313 | 38.193 | 1.00 | 107.84 | 8 |
| ATOM | 12816 | N | ARG | F | 475 | 112.505 | 92.155 | 36.675 | 1.00 | 107.45 | 7 |
| ATOM | 12817 | CA | ARG | F | 475 | 111.527 | 93.224 | 36.480 | 1.00 | 107.51 | 6 |
| ATOM | 12818 | CB | ARG | F | 475 | 110.458 | 92.787 | 35.474 | 1.00 | 141.99 | 6 |
| ATOM | 12819 | CG | ARG | F | 475 | 110.974 | 92.448 | 34.080 | 1.00 | 141.55 | 6 |
| ATOM | 12820 | CD | ARG | F | 475 | 111.535 | 93.667 | 33.359 | 1.00 | 141.54 | 6 |
| ATOM | 12821 | NE | ARG | F | 475 | 111.576 | 93.458 | 31.913 | 1.00 | 141.93 | 7 |
| ATOM | 12822 | CZ | ARG | F | 475 | 111.971 | 94.371 | 31.031 | 1.00 | 141.54 | 6 |
| ATOM | 12823 | NH1 | ARG | F | 475 | 112.367 | 95.567 | 31.441 | 1.00 | 141.34 | 7 |
| ATOM | 12824 | NH2 | ARG | F | 475 | 111.958 | 94.089 | 29.735 | 1.00 | 141.23 | 7 |
| ATOM | 12825 | C | ARG | F | 475 | 110.861 | 93.542 | 37.822 | 1.00 | 107.45 | 6 |
| ATOM | 12826 | O | ARG | F | 475 | 110.295 | 94.621 | 38.021 | 1.00 | 107.57 | 8 |
| ATOM | 12827 | N | ALA | F | 476 | 110.935 | 92.583 | 38.738 | 1.00 | 99.87 | 7 |
| ATOM | 12828 | CA | ALA | F | 476 | 110.351 | 92.720 | 40.065 | 1.00 | 99.52 | 6 |
| ATOM | 12829 | CB | ALA | F | 476 | 110.477 | 91.395 | 40.823 | 1.00 | 23.91 | 6 |
| ATOM | 12830 | C | ALA | F | 476 | 111.012 | 93.848 | 40.858 | 1.00 | 99.38 | 6 |
| ATOM | 12831 | O | ALA | F | 476 | 110.378 | 94.870 | 41.141 | 1.00 | 99.48 | 8 |
| ATOM | 12832 | N | LEU | F | 477 | 112.282 | 93.656 | 41.218 | 1.00 | 83.50 | 7 |
| ATOM | 12833 | CA | LEU | F | 477 | 113.022 | 94.657 | 41.980 | 1.00 | 83.52 | 6 |
| ATOM | 12834 | CB | LEU | F | 477 | 114.353 | 94.090 | 42.469 | 1.00 | 91.72 | 6 |
| ATOM | 12835 | CG | LEU | F | 477 | 115.096 | 94.997 | 43.458 | 1.00 | 92.35 | 6 |
| ATOM | 12836 | CD1 | LEU | F | 477 | 114.213 | 95.278 | 44.671 | 1.00 | 92.01 | 6 |
| ATOM | 12837 | CD2 | LEU | F | 477 | 116.408 | 94.338 | 43.888 | 1.00 | 92.88 | 6 |
| ATOM | 12838 | C | LEU | F | 477 | 113.272 | 95.863 | 41.096 | 1.00 | 83.85 | 6 |
| ATOM | 12839 | O | LEU | F | 477 | 113.595 | 96.950 | 41.581 | 1.00 | 83.05 | 8 |
| ATOM | 12840 | N | ALA | F | 478 | 113.120 | 95.656 | 39.792 | 1.00 | 145.48 | 7 |
| ATOM | 12841 | CA | ALA | F | 478 | 113.297 | 96.720 | 38.816 | 1.00 | 147.27 | 6 |
| ATOM | 12842 | CB | ALA | F | 478 | 113.356 | 96.139 | 37.412 | 1.00 | 126.87 | 6 |
| ATOM | 12843 | C | ALA | F | 478 | 112.096 | 97.641 | 38.952 | 1.00 | 148.37 | 6 |
| ATOM | 12844 | O | ALA | F | 478 | 112.232 | 98.821 | 39.277 | 1.00 | 148.44 | 8 |
| ATOM | 12845 | N | ALA | F | 479 | 110.915 | 97.089 | 38.702 | 1.00 | 136.33 | 7 |
| ATOM | 12846 | CA | ALA | F | 479 | 109.687 | 97.855 | 38.820 | 1.00 | 137.64 | 6 |
| ATOM | 12847 | CB | ALA | F | 479 | 108.482 | 96.929 | 38.660 | 1.00 | 117.85 | 6 |
| ATOM | 12848 | C | ALA | F | 479 | 109.693 | 98.493 | 40.207 | 1.00 | 138.59 | 6 |
| ATOM | 12849 | O | ALA | F | 479 | 109.482 | 99.699 | 40.357 | 1.00 | 139.42 | 8 |
| ATOM | 12850 | N | GLU | F | 480 | 109.958 | 97.668 | 41.216 | 1.00 | 152.44 | 7 |
| ATOM | 12851 | CA | GLU | F | 480 | 110.010 | 98.119 | 42.600 | 1.00 | 153.69 | 6 |
| ATOM | 12852 | CB | GLU | F | 480 | 110.554 | 96.996 | 43.492 | 1.00 | 169.11 | 6 |
| ATOM | 12853 | CG | GLU | F | 480 | 110.873 | 97.411 | 44.922 | 1.00 | 170.48 | 6 |
| ATOM | 12854 | CD | GLU | F | 480 | 109.646 | 97.826 | 45.706 | 1.00 | 171.78 | 6 |
| ATOM | 12855 | OE1 | GLU | F | 480 | 108.756 | 96.977 | 45.909 | 1.00 | 172.92 | 8 |
| ATOM | 12856 | OE2 | GLU | F | 480 | 109.572 | 99.001 | 46.123 | 1.00 | 172.97 | 8 |
| ATOM | 12857 | C | GLU | F | 480 | 110.889 | 99.359 | 42.731 | 1.00 | 154.65 | 6 |
| ATOM | 12858 | O | GLU | F | 480 | 110.735 | 100.141 | 43.669 | 1.00 | 154.26 | 8 |
| ATOM | 12859 | N | MET | F | 481 | 111.807 | 99.541 | 41.787 | 1.00 | 171.53 | 7 |
| ATOM | 12860 | CA | MET | F | 481 | 112.698 | 100.692 | 41.833 | 1.00 | 172.59 | 6 |
| ATOM | 12861 | CB | MET | F | 481 | 114.032 | 100.377 | 41.145 | 1.00 | 195.46 | 6 |
| ATOM | 12862 | CG | MET | F | 481 | 115.126 | 101.411 | 41.420 | 1.00 | 197.17 | 6 |
| ATOM | 12863 | SD | MET | F | 481 | 115.700 | 102.328 | 39.968 | 1.00 | 198.29 | 16 |
| ATOM | 12864 | CE | MET | F | 481 | 117.184 | 101.401 | 39.565 | 1.00 | 198.23 | 6 |
| ATOM | 12865 | C | MET | F | 481 | 112.068 | 101.917 | 41.186 | 1.00 | 172.32 | 6 |
| ATOM | 12866 | O | MET | F | 481 | 112.696 | 102.974 | 41.115 | 1.00 | 172.57 | 8 |
| ATOM | 12867 | N | LYS | F | 482 | 110.833 | 101.787 | 40.712 | 1.00 | 125.11 | 7 |
| ATOM | 12868 | CA | LYS | F | 482 | 110.182 | 102.933 | 40.101 | 1.00 | 125.70 | 6 |
| ATOM | 12869 | CB | LYS | F | 482 | 109.566 | 102.577 | 38.750 | 1.00 | 123.80 | 6 |
| ATOM | 12870 | CG | LYS | F | 482 | 109.282 | 103.809 | 37.895 | 1.00 | 124.10 | 6 |
| ATOM | 12871 | CD | LYS | F | 482 | 109.064 | 103.435 | 36.443 | 1.00 | 124.93 | 6 |
| ATOM | 12872 | CE | LYS | F | 482 | 109.085 | 104.660 | 35.549 | 1.00 | 125.42 | 6 |
| ATOM | 12873 | NZ | LYS | F | 482 | 109.040 | 104.274 | 34.109 | 1.00 | 125.58 | 7 |
| ATOM | 12874 | C | LYS | F | 482 | 109.124 | 103.519 | 41.018 | 1.00 | 125.74 | 6 |
| ATOM | 12875 | O | LYS | F | 482 | 108.029 | 102.980 | 41.176 | 1.00 | 125.86 | 8 |
| ATOM | 12876 | N | ALA | F | 483 | 109.495 | 104.636 | 41.625 | 1.00 | 201.16 | 7 |
| ATOM | 12877 | CA | ALA | F | 483 | 108.658 | 105.380 | 42.543 | 1.00 | 200.45 | 6 |
| ATOM | 12878 | CB | ALA | F | 483 | 108.714 | 104.750 | 43.930 | 1.00 | 57.23 | 6 |
| ATOM | 12879 | C | ALA | F | 483 | 109.169 | 106.820 | 42.592 | 1.00 | 200.60 | 6 |
| ATOM | 12880 | O | ALA | F | 483 | 108.389 | 107.752 | 42.793 | 1.00 | 201.18 | 8 |
| ATOM | 12881 | N | PRO | F | 484 | 110.490 | 107.025 | 42.397 | 1.00 | 128.83 | 7 |
| ATOM | 12882 | CD | PRO | F | 484 | 111.546 | 106.096 | 41.960 | 1.00 | 163.61 | 6 |
| ATOM | 12883 | CA | PRO | F | 484 | 111.012 | 108.392 | 42.437 | 1.00 | 128.74 | 6 |
| ATOM | 12884 | CB | PRO | F | 484 | 112.467 | 108.221 | 42.000 | 1.00 | 163.95 | 6 |
| ATOM | 12885 | CG | PRO | F | 484 | 112.427 | 106.998 | 41.144 | 1.00 | 163.51 | 6 |
| ATOM | 12886 | C | PRO | F | 484 | 110.234 | 109.313 | 41.516 | 1.00 | 128.71 | 6 |
| ATOM | 12887 | O | PRO | F | 484 | 109.676 | 110.315 | 41.959 | 1.00 | 128.57 | 8 |
| ATOM | 12888 | N | SER | F | 485 | 110.193 | 108.963 | 40.236 | 1.00 | 110.07 | 7 |
| ATOM | 12889 | CA | SER | F | 485 | 109.470 | 109.762 | 39.258 | 1.00 | 110.23 | 6 |
| ATOM | 12890 | CB | SER | F | 485 | 109.391 | 109.015 | 37.925 | 1.00 | 126.40 | 6 |
| ATOM | 12891 | OG | SER | F | 485 | 108.681 | 109.770 | 36.960 | 1.00 | 126.46 | 8 |
| ATOM | 12892 | C | SER | F | 485 | 108.063 | 110.087 | 39.767 | 1.00 | 110.37 | 6 |
| ATOM | 12893 | O | SER | F | 485 | 107.555 | 109.442 | 40.688 | 1.00 | 110.69 | 8 |
| ATOM | 12894 | N | ARG | F | 486 | 107.444 | 111.095 | 39.161 | 1.00 | 108.29 | 7 |
| ATOM | 12895 | CA | ARG | F | 486 | 106.108 | 111.538 | 39.542 | 1.00 | 108.04 | 6 |
| ATOM | 12896 | CB | ARG | F | 486 | 105.807 | 112.883 | 38.878 | 1.00 | 191.80 | 6 |
| ATOM | 12897 | CG | ARG | F | 486 | 106.120 | 112.937 | 37.386 | 1.00 | 191.38 | 6 |
| ATOM | 12898 | CD | ARG | F | 486 | 105.239 | 113.968 | 36.703 | 1.00 | 190.89 | 6 |
| ATOM | 12899 | NE | ARG | F | 486 | 105.201 | 115.223 | 37.449 | 1.00 | 190.38 | 7 |
| ATOM | 12900 | CZ | ARG | F | 486 | 104.299 | 116.178 | 37.255 | 1.00 | 190.43 | 6 |
| ATOM | 12901 | NH1 | ARG | F | 486 | 103.355 | 116.024 | 36.336 | 1.00 | 190.60 | 7 |
| ATOM | 12902 | NH2 | ARG | F | 486 | 104.334 | 117.285 | 37.986 | 1.00 | 190.17 | 7 |
| ATOM | 12903 | C | ARG | F | 486 | 104.972 | 110.559 | 39.229 | 1.00 | 108.40 | 6 |
| ATOM | 12904 | O | ARG | F | 486 | 104.580 | 109.756 | 40.077 | 1.00 | 108.25 | 8 |
| ATOM | 12905 | N | ALA | F | 487 | 104.440 | 110.641 | 38.012 | 1.00 | 208.87 | 7 |
| ATOM | 12906 | CA | ALA | F | 487 | 103.332 | 109.789 | 37.582 | 1.00 | 208.87 | 6 |
| ATOM | 12907 | CB | ALA | F | 487 | 102.559 | 110.480 | 36.452 | 1.00 | 163.19 | 6 |
| ATOM | 12908 | C | ALA | F | 487 | 103.764 | 108.388 | 37.143 | 1.00 | 208.87 | 6 |
| ATOM | 12909 | O | ALA | F | 487 | 103.075 | 107.403 | 37.421 | 1.00 | 208.87 | 8 |
| ATOM | 12910 | N | ARG | F | 488 | 104.898 | 108.301 | 36.454 | 1.00 | 183.84 | 7 |
| ATOM | 12911 | CA | ARG | F | 488 | 105.415 | 107.019 | 35.979 | 1.00 | 183.25 | 6 |
| ATOM | 12912 | CB | ARG | F | 488 | 106.778 | 107.225 | 35.318 | 1.00 | 195.91 | 6 |
| ATOM | 12913 | CG | ARG | F | 488 | 106.778 | 108.271 | 34.214 | 1.00 | 196.60 | 6 |
| ATOM | 12914 | CD | ARG | F | 488 | 108.187 | 108.517 | 33.696 | 1.00 | 197.72 | 6 |
| ATOM | 12915 | NE | ARG | F | 488 | 108.222 | 109.500 | 32.617 | 1.00 | 198.81 | 7 |
| ATOM | 12916 | CZ | ARG | F | 488 | 109.334 | 109.899 | 32.006 | 1.00 | 199.34 | 6 |
| ATOM | 12917 | NH1 | ARG | F | 488 | 110.510 | 109.400 | 32.367 | 1.00 | 200.18 | 7 |
| ATOM | 12918 | NH2 | ARG | F | 488 | 109.272 | 110.794 | 31.030 | 1.00 | 199.44 | 7 |
| ATOM | 12919 | C | ARG | F | 488 | 105.547 | 106.029 | 37.133 | 1.00 | 182.60 | 6 |
| ATOM | 12920 | O | ARG | F | 488 | 105.600 | 104.816 | 36.926 | 1.00 | 182.59 | 8 |
| ATOM | 12921 | N | ARG | F | 489 | 105.601 | 106.568 | 38.348 | 1.00 | 194.53 | 7 |
| ATOM | 12922 | CA | ARG | F | 489 | 105.728 | 105.772 | 39.563 | 1.00 | 193.24 | 6 |
| ATOM | 12923 | CB | ARG | F | 489 | 105.595 | 106.668 | 40.799 | 1.00 | 207.41 | 6 |
| ATOM | 12924 | CG | ARG | F | 489 | 105.414 | 105.899 | 42.103 | 1.00 | 208.86 | 6 |
| ATOM | 12925 | CD | ARG | F | 489 | 104.827 | 106.782 | 43.196 | 1.00 | 208.87 | 6 |
| ATOM | 12926 | NE | ARG | F | 489 | 104.466 | 106.014 | 44.386 | 1.00 | 208.87 | 7 |
| ATOM | 12927 | CZ | ARG | F | 489 | 103.763 | 106.498 | 45.408 | 1.00 | 208.87 | 6 |
| ATOM | 12928 | NH1 | ARG | F | 489 | 103.338 | 107.756 | 45.392 | 1.00 | 208.87 | 7 |
| ATOM | 12929 | NH2 | ARG | F | 489 | 103.481 | 105.721 | 46.445 | 1.00 | 208.87 | 7 |
| ATOM | 12930 | C | ARG | F | 489 | 104.672 | 104.683 | 39.635 | 1.00 | 191.50 | 6 |
| ATOM | 12931 | O | ARG | F | 489 | 104.951 | 103.562 | 40.056 | 1.00 | 191.14 | 8 |
| ATOM | 12932 | N | ALA | F | 490 | 103.458 | 105.021 | 39.218 | 1.00 | 104.12 | 7 |
| ATOM | 12933 | CA | ALA | F | 490 | 102.345 | 104.082 | 39.260 | 1.00 | 102.04 | 6 |
| ATOM | 12934 | CB | ALA | F | 490 | 101.030 | 104.853 | 39.236 | 1.00 | 167.05 | 6 |
| ATOM | 12935 | C | ALA | F | 490 | 102.359 | 103.041 | 38.146 | 1.00 | 100.50 | 6 |
| ATOM | 12936 | O | ALA | F | 490 | 102.230 | 101.843 | 38.406 | 1.00 | 99.85 | 8 |
| ATOM | 12937 | N | LYS | F | 491 | 102.506 | 103.502 | 36.908 | 1.00 | 76.30 | 7 |
| ATOM | 12938 | CA | LYS | F | 491 | 102.517 | 102.608 | 35.757 | 1.00 | 75.17 | 6 |
| ATOM | 12939 | CB | LYS | F | 491 | 103.203 | 103.293 | 34.568 | 1.00 | 127.19 | 6 |
| ATOM | 12940 | CG | LYS | F | 491 | 103.182 | 102.472 | 33.281 | 1.00 | 127.46 | 6 |
| ATOM | 12941 | CD | LYS | F | 491 | 103.762 | 103.244 | 32.094 | 1.00 | 127.22 | 6 |
| ATOM | 12942 | CE | LYS | F | 491 | 103.773 | 102.395 | 30.823 | 1.00 | 126.76 | 6 |
| ATOM | 12943 | NZ | LYS | F | 491 | 104.299 | 103.134 | 29.642 | 1.00 | 125.81 | 7 |
| ATOM | 12944 | C | LYS | F | 491 | 103.207 | 101.284 | 36.077 | 1.00 | 74.50 | 6 |
| ATOM | 12945 | O | LYS | F | 491 | 102.566 | 100.234 | 36.120 | 1.00 | 74.45 | 8 |
| ATOM | 12946 | N | ALA | F | 492 | 104.513 | 101.339 | 36.317 | 1.00 | 154.72 | 7 |
| ATOM | 12947 | CA | ALA | F | 492 | 105.276 | 100.138 | 36.627 | 1.00 | 153.72 | 6 |
| ATOM | 12948 | CB | ALA | F | 492 | 106.727 | 100.507 | 36.957 | 1.00 | 87.63 | 6 |
| ATOM | 12949 | C | ALA | F | 492 | 104.650 | 99.352 | 37.780 | 1.00 | 152.88 | 6 |
| ATOM | 12950 | O | ALA | F | 492 | 104.627 | 98.123 | 37.761 | 1.00 | 153.45 | 8 |
| ATOM | 12951 | N | ARG | F | 493 | 104.131 | 100.056 | 38.778 | 1.00 | 91.36 | 7 |
| ATOM | 12952 | CA | ARG | F | 493 | 103.521 | 99.390 | 39.922 | 1.00 | 90.04 | 6 |
| ATOM | 12953 | CB | ARG | F | 493 | 103.115 | 100.417 | 40.974 | 1.00 | 125.67 | 6 |
| ATOM | 12954 | CG | ARG | F | 493 | 104.294 | 101.163 | 41.552 | 1.00 | 125.72 | 6 |
| ATOM | 12955 | CD | ARG | F | 493 | 105.274 | 100.217 | 42.222 | 1.00 | 125.76 | 6 |
| ATOM | 12956 | NE | ARG | F | 493 | 106.494 | 100.917 | 42.602 | 1.00 | 126.66 | 7 |
| ATOM | 12957 | CZ | ARG | F | 493 | 107.459 | 100.392 | 43.347 | 1.00 | 127.10 | 6 |
| ATOM | 12958 | NH1 | ARG | F | 493 | 107.350 | 99.150 | 43.800 | 1.00 | 126.62 | 7 |
| ATOM | 12959 | NH2 | ARG | F | 493 | 108.531 | 101.114 | 43.638 | 1.00 | 127.67 | 7 |
| ATOM | 12960 | C | ARG | F | 493 | 102.320 | 98.538 | 39.541 | 1.00 | 89.07 | 6 |
| ATOM | 12961 | O | ARG | F | 493 | 102.153 | 97.434 | 40.055 | 1.00 | 89.29 | 8 |
| ATOM | 12962 | N | LYS | F | 494 | 101.471 | 99.049 | 38.654 | 1.00 | 87.69 | 7 |
| ATOM | 12963 | CA | LYS | F | 494 | 100.309 | 98.277 | 38.227 | 1.00 | 85.89 | 6 |
| ATOM | 12964 | CB | LYS | F | 494 | 99.505 | 99.027 | 37.158 | 1.00 | 135.77 | 6 |
| ATOM | 12965 | CG | LYS | F | 494 | 98.483 | 98.159 | 36.416 | 1.00 | 136.55 | 6 |
| ATOM | 12966 | CD | LYS | F | 494 | 97.562 | 97.405 | 37.374 | 1.00 | 137.12 | 6 |
| ATOM | 12967 | CE | LYS | F | 494 | 96.638 | 96.450 | 36.629 | 1.00 | 136.57 | 6 |
| ATOM | 12968 | NZ | LYS | F | 494 | 95.858 | 95.587 | 37.559 | 1.00 | 136.70 | 7 |
| ATOM | 12969 | C | LYS | F | 494 | 100.847 | 96.978 | 37.661 | 1.00 | 83.95 | 6 |
| ATOM | 12970 | O | LYS | F | 494 | 100.259 | 95.913 | 37.852 | 1.00 | 83.51 | 8 |
| ATOM | 12971 | N | ARG | F | 495 | 101.975 | 97.078 | 36.964 | 1.00 | 55.10 | 7 |
| ATOM | 12972 | CA | ARG | F | 495 | 102.607 | 95.901 | 36.402 | 1.00 | 53.32 | 6 |
| ATOM | 12973 | CB | ARG | F | 495 | 103.890 | 96.272 | 35.677 | 1.00 | 88.28 | 6 |
| ATOM | 12974 | CG | ARG | F | 495 | 103.732 | 97.313 | 34.596 | 1.00 | 89.56 | 6 |
| ATOM | 12975 | CD | ARG | F | 495 | 104.991 | 97.321 | 33.753 | 1.00 | 90.19 | 6 |
| ATOM | 12976 | NE | ARG | F | 495 | 104.966 | 98.309 | 32.686 | 1.00 | 91.11 | 7 |
| ATOM | 12977 | CZ | ARG | F | 495 | 105.882 | 98.367 | 31.730 | 1.00 | 92.51 | 6 |
| ATOM | 12978 | NH1 | ARG | F | 495 | 106.879 | 97.488 | 31.724 | 1.00 | 92.23 | 7 |
| ATOM | 12979 | NH2 | ARG | F | 495 | 105.809 | 99.303 | 30.791 | 1.00 | 93.51 | 7 |
| ATOM | 12980 | C | ARG | F | 495 | 102.944 | 95.003 | 37.581 | 1.00 | 51.67 | 6 |
| ATOM | 12981 | O | ARG | F | 495 | 102.719 | 93.801 | 37.546 | 1.00 | 50.96 | 8 |
| ATOM | 12982 | N | LEU | F | 496 | 103.487 | 95.607 | 38.631 | 1.00 | 63.99 | 7 |
| ATOM | 12983 | CA | LEU | F | 496 | 103.847 | 94.868 | 39.828 | 1.00 | 63.47 | 6 |
| ATOM | 12984 | CB | LEU | F | 496 | 104.200 | 95.843 | 40.953 | 1.00 | 65.63 | 6 |
| ATOM | 12985 | CG | LEU | F | 496 | 104.639 | 95.302 | 42.316 | 1.00 | 65.50 | 6 |
| ATOM | 12986 | CD1 | LEU | F | 496 | 105.927 | 94.498 | 42.213 | 1.00 | 65.71 | 6 |
| ATOM | 12987 | CD2 | LEU | F | 496 | 104.838 | 96.485 | 43.231 | 1.00 | 65.48 | 6 |
| ATOM | 12988 | C | LEU | F | 496 | 102.656 | 93.996 | 40.216 | 1.00 | 63.38 | 6 |
| ATOM | 12989 | O | LEU | F | 496 | 102.814 | 92.828 | 40.570 | 1.00 | 63.43 | 8 |
| ATOM | 12990 | N | GLU | F | 497 | 101.456 | 94.560 | 40.139 | 1.00 | 122.41 | 7 |
| ATOM | 12991 | CA | GLU | F | 497 | 100.265 | 93.789 | 40.471 | 1.00 | 122.58 | 6 |
| ATOM | 12992 | CB | GLU | F | 497 | 98.996 | 94.636 | 40.296 | 1.00 | 133.57 | 6 |
| ATOM | 12993 | CG | GLU | F | 497 | 98.753 | 95.631 | 41.428 | 1.00 | 135.30 | 6 |
| ATOM | 12994 | CD | GLU | F | 497 | 97.354 | 96.224 | 41.402 | 1.00 | 135.97 | 6 |
| ATOM | 12995 | OE1 | GLU | F | 497 | 97.065 | 97.047 | 40.507 | 1.00 | 136.03 | 8 |
| ATOM | 12996 | OE2 | GLU | F | 497 | 96.541 | 95.862 | 42.279 | 1.00 | 137.23 | 8 |
| ATOM | 12997 | C | GLU | F | 497 | 100.218 | 92.573 | 39.553 | 1.00 | 122.13 | 6 |
| ATOM | 12998 | O | GLU | F | 497 | 100.154 | 91.436 | 40.017 | 1.00 | 122.56 | 8 |
| ATOM | 12999 | N | VAL | F | 498 | 100.272 | 92.828 | 38.250 | 1.00 | 72.19 | 7 |
| ATOM | 13000 | CA | VAL | F | 498 | 100.242 | 91.774 | 37.249 | 1.00 | 70.74 | 6 |
| ATOM | 13001 | CB | VAL | F | 498 | 100.221 | 92.378 | 35.824 | 1.00 | 72.47 | 6 |
| ATOM | 13002 | CG1 | VAL | F | 498 | 99.927 | 91.300 | 34.799 | 1.00 | 72.74 | 6 |
| ATOM | 13003 | CG2 | VAL | F | 498 | 99.191 | 93.484 | 35.744 | 1.00 | 72.28 | 6 |
| ATOM | 13004 | C | VAL | F | 498 | 101.480 | 90.891 | 37.395 | 1.00 | 70.14 | 6 |
| ATOM | 13005 | O | VAL | F | 498 | 101.379 | 89.669 | 37.478 | 1.00 | 70.06 | 8 |
| ATOM | 13006 | N | VAL | F | 499 | 102.648 | 91.527 | 37.426 | 1.00 | 55.74 | 7 |
| ATOM | 13007 | CA | VAL | F | 499 | 103.923 | 90.831 | 37.550 | 1.00 | 54.82 | 6 |
| ATOM | 13008 | CB | VAL | F | 499 | 105.093 | 91.815 | 37.601 | 1.00 | 43.37 | 6 |
| ATOM | 13009 | CG1 | VAL | F | 499 | 106.377 | 91.067 | 37.945 | 1.00 | 43.47 | 6 |
| ATOM | 13010 | CG2 | VAL | F | 499 | 105.211 | 92.548 | 36.268 | 1.00 | 42.33 | 6 |
| ATOM | 13011 | C | VAL | F | 499 | 103.971 | 89.982 | 38.801 | 1.00 | 55.59 | 6 |
| ATOM | 13012 | O | VAL | F | 499 | 104.171 | 88.775 | 38.724 | 1.00 | 55.51 | 8 |
| ATOM | 13013 | N | ARG | F | 500 | 103.809 | 90.614 | 39.959 | 1.00 | 56.20 | 7 |
| ATOM | 13014 | CA | ARG | F | 500 | 103.823 | 89.859 | 41.199 | 1.00 | 57.29 | 6 |
| ATOM | 13015 | CB | ARG | F | 500 | 103.587 | 90.764 | 42.416 | 1.00 | 104.33 | 6 |
| ATOM | 13016 | CG | ARG | F | 500 | 104.844 | 91.519 | 42.863 | 1.00 | 105.86 | 6 |
| ATOM | 13017 | CD | ARG | F | 500 | 106.035 | 90.560 | 43.038 | 1.00 | 107.04 | 6 |
| ATOM | 13018 | NE | ARG | F | 500 | 107.312 | 91.247 | 43.251 | 1.00 | 107.51 | 7 |
| ATOM | 13019 | CZ | ARG | F | 500 | 108.489 | 90.633 | 43.380 | 1.00 | 107.98 | 6 |
| ATOM | 13020 | NH1 | ARG | F | 500 | 108.574 | 89.310 | 43.319 | 1.00 | 108.10 | 7 |
| ATOM | 13021 | NH2 | ARG | F | 500 | 109.591 | 91.342 | 43.578 | 1.00 | 108.12 | 7 |
| ATOM | 13022 | C | ARG | F | 500 | 102.731 | 88.814 | 41.079 | 1.00 | 57.39 | 6 |
| ATOM | 13023 | O | ARG | F | 500 | 102.906 | 87.671 | 41.501 | 1.00 | 57.07 | 8 |
| ATOM | 13024 | N | ALA | F | 501 | 101.611 | 89.201 | 40.477 | 1.00 | 75.03 | 7 |
| ATOM | 13025 | CA | ALA | F | 501 | 100.513 | 88.263 | 40.282 | 1.00 | 75.89 | 6 |
| ATOM | 13026 | CB | ALA | F | 501 | 99.393 | 88.910 | 39.467 | 1.00 | 97.61 | 6 |
| ATOM | 13027 | C | ALA | F | 501 | 101.091 | 87.075 | 39.528 | 1.00 | 75.95 | 6 |
| ATOM | 13028 | O | ALA | F | 501 | 101.143 | 85.955 | 40.042 | 1.00 | 75.79 | 8 |
| ATOM | 13029 | N | PHE | F | 502 | 101.535 | 87.338 | 38.305 | 1.00 | 72.31 | 7 |
| ATOM | 13030 | CA | PHE | F | 502 | 102.119 | 86.305 | 37.479 | 1.00 | 73.66 | 6 |
| ATOM | 13031 | CB | PHE | F | 502 | 102.751 | 86.923 | 36.231 | 1.00 | 106.46 | 6 |
| ATOM | 13032 | CG | PHE | F | 502 | 101.938 | 86.719 | 34.992 | 1.00 | 107.25 | 6 |
| ATOM | 13033 | CD1 | PHE | F | 502 | 100.591 | 87.043 | 34.976 | 1.00 | 107.94 | 6 |
| ATOM | 13034 | CD2 | PHE | F | 502 | 102.498 | 86.152 | 33.858 | 1.00 | 107.10 | 6 |
| ATOM | 13035 | CE1 | PHE | F | 502 | 99.809 | 86.799 | 33.853 | 1.00 | 108.27 | 6 |
| ATOM | 13036 | CE2 | PHE | F | 502 | 101.721 | 85.905 | 32.730 | 1.00 | 107.18 | 6 |
| ATOM | 13037 | CZ | PHE | F | 502 | 100.373 | 86.229 | 32.732 | 1.00 | 107.70 | 6 |
| ATOM | 13038 | C | PHE | F | 502 | 103.159 | 85.581 | 38.298 | 1.00 | 74.40 | 6 |
| ATOM | 13039 | O | PHE | F | 502 | 102.986 | 84.417 | 38.659 | 1.00 | 75.35 | 8 |
| ATOM | 13040 | N | LEU | F | 503 | 104.227 | 86.300 | 38.612 | 1.00 | 144.01 | 7 |
| ATOM | 13041 | CA | LEU | F | 503 | 105.341 | 85.777 | 39.385 | 1.00 | 143.30 | 6 |
| ATOM | 13042 | CB | LEU | F | 503 | 106.236 | 86.941 | 39.812 | 1.00 | 62.42 | 6 |
| ATOM | 13043 | CG | LEU | F | 503 | 107.595 | 86.642 | 40.432 | 1.00 | 61.59 | 6 |
| ATOM | 13044 | CD1 | LEU | F | 503 | 108.292 | 85.474 | 39.727 | 1.00 | 61.85 | 6 |
| ATOM | 13045 | CD2 | LEU | F | 503 | 108.416 | 87.913 | 40.343 | 1.00 | 61.12 | 6 |
| ATOM | 13046 | C | LEU | F | 503 | 104.901 | 84.966 | 40.601 | 1.00 | 143.57 | 6 |
| ATOM | 13047 | O | LEU | F | 503 | 105.648 | 84.126 | 41.101 | 1.00 | 143.43 | 8 |
| ATOM | 13048 | N | ASP | F | 504 | 103.684 | 85.212 | 41.070 | 1.00 | 65.46 | 7 |
| ATOM | 13049 | CA | ASP | F | 504 | 103.169 | 84.494 | 42.225 | 1.00 | 65.60 | 6 |
| ATOM | 13050 | CB | ASP | F | 504 | 102.127 | 85.344 | 42.953 | 1.00 | 116.60 | 6 |
| ATOM | 13051 | CG | ASP | F | 504 | 101.593 | 84.671 | 44.202 | 1.00 | 117.92 | 6 |
| ATOM | 13052 | OD1 | ASP | F | 504 | 100.415 | 84.246 | 44.201 | 1.00 | 118.18 | 8 |
| ATOM | 13053 | OD2 | ASP | F | 504 | 102.356 | 84.568 | 45.187 | 1.00 | 118.37 | 8 |
| ATOM | 13054 | C | ASP | F | 504 | 102.550 | 83.180 | 41.790 | 1.00 | 65.38 | 6 |
| ATOM | 13055 | O | ASP | F | 504 | 101.956 | 83.086 | 40.711 | 1.00 | 64.75 | 8 |
| ATOM | 13056 | N | SER | F | 505 | 102.689 | 82.169 | 42.640 | 1.00 | 171.49 | 7 |
| ATOM | 13057 | CA | SER | F | 505 | 102.148 | 80.846 | 42.358 | 1.00 | 172.26 | 6 |
| ATOM | 13058 | CB | SER | F | 505 | 100.621 | 80.870 | 42.392 | 1.00 | 142.22 | 6 |
| ATOM | 13059 | OG | SER | F | 505 | 100.103 | 81.468 | 41.213 | 1.00 | 142.34 | 8 |
| ATOM | 13060 | C | SER | F | 505 | 102.594 | 80.417 | 40.974 | 1.00 | 172.09 | 6 |
| ATOM | 13061 | O | SER | F | 505 | 103.662 | 80.809 | 40.502 | 1.00 | 173.16 | 8 |
| ATOM | 13062 | N | GLY | F | 506 | 101.756 | 79.621 | 40.322 | 1.00 | 136.01 | 7 |
| ATOM | 13063 | CA | GLY | F | 506 | 102.073 | 79.153 | 38.991 | 1.00 | 135.48 | 6 |
| ATOM | 13064 | C | GLY | F | 506 | 102.068 | 80.270 | 37.968 | 1.00 | 134.91 | 6 |
| ATOM | 13065 | O | GLY | F | 506 | 102.060 | 81.454 | 38.317 | 1.00 | 134.80 | 8 |
| ATOM | 13066 | N | ASN | F | 507 | 102.068 | 79.867 | 36.701 | 1.00 | 83.16 | 7 |
| ATOM | 13067 | CA | ASN | F | 507 | 102.071 | 80.758 | 35.548 | 1.00 | 81.70 | 6 |
| ATOM | 13068 | CB | ASN | F | 507 | 101.599 | 82.175 | 35.885 | 1.00 | 73.42 | 6 |
| ATOM | 13069 | CG | ASN | F | 507 | 101.890 | 83.169 | 34.755 | 1.00 | 72.57 | 6 |
| ATOM | 13070 | OD1 | ASN | F | 507 | 101.037 | 83.458 | 33.917 | 1.00 | 72.40 | 8 |
| ATOM | 13071 | ND2 | ASN | F | 507 | 103.112 | 83.674 | 34.726 | 1.00 | 72.19 | 7 |
| ATOM | 13072 | C | ASN | F | 507 | 103.445 | 80.881 | 34.951 | 1.00 | 80.94 | 6 |
| ATOM | 13073 | O | ASN | F | 507 | 104.454 | 80.812 | 35.646 | 1.00 | 81.65 | 8 |
| ATOM | 13074 | N | ARG | F | 508 | 103.441 | 81.075 | 33.641 | 1.00 | 31.98 | 7 |
| ATOM | 13075 | CA | ARG | F | 508 | 104.609 | 81.268 | 32.808 | 1.00 | 31.15 | 6 |
| ATOM | 13076 | CB | ARG | F | 508 | 105.322 | 79.939 | 32.520 | 1.00 | 80.17 | 6 |
| ATOM | 13077 | CG | ARG | F | 508 | 105.405 | 78.991 | 33.711 | 1.00 | 81.68 | 6 |
| ATOM | 13078 | CD | ARG | F | 508 | 106.294 | 77.766 | 33.470 | 1.00 | 82.38 | 6 |
| ATOM | 13079 | NE | ARG | F | 508 | 106.313 | 76.885 | 34.644 | 1.00 | 82.32 | 7 |
| ATOM | 13080 | CZ | ARG | F | 508 | 107.069 | 75.795 | 34.768 | 1.00 | 82.37 | 6 |
| ATOM | 13081 | NH1 | ARG | F | 508 | 107.891 | 75.432 | 33.788 | 1.00 | 81.92 | 7 |
| ATOM | 13082 | NH2 | ARG | F | 508 | 106.994 | 75.060 | 35.872 | 1.00 | 81.71 | 7 |
| ATOM | 13083 | C | ARG | F | 508 | 103.811 | 81.686 | 31.595 | 1.00 | 30.98 | 6 |
| ATOM | 13084 | O | ARG | F | 508 | 103.053 | 80.880 | 31.084 | 1.00 | 30.86 | 8 |
| ATOM | 13085 | N | PRO | F | 509 | 103.905 | 82.953 | 31.158 | 1.00 | 57.22 | 7 |
| ATOM | 13086 | CD | PRO | F | 509 | 104.693 | 84.033 | 31.771 | 1.00 | 45.48 | 6 |
| ATOM | 13087 | CA | PRO | F | 509 | 103.169 | 83.451 | 29.986 | 1.00 | 57.41 | 6 |
| ATOM | 13088 | CB | PRO | F | 509 | 103.974 | 84.667 | 29.576 | 1.00 | 44.60 | 6 |
| ATOM | 13089 | CG | PRO | F | 509 | 104.334 | 85.238 | 30.903 | 1.00 | 45.30 | 6 |
| ATOM | 13090 | C | PRO | F | 509 | 103.021 | 82.452 | 28.838 | 1.00 | 58.61 | 6 |
| ATOM | 13091 | O | PRO | F | 509 | 103.623 | 82.612 | 27.775 | 1.00 | 58.75 | 8 |
| ATOM | 13092 | N | GLU | F | 510 | 102.212 | 81.423 | 29.075 | 1.00 | 137.55 | 7 |
| ATOM | 13093 | CA | GLU | F | 510 | 101.943 | 80.376 | 28.102 | 1.00 | 138.27 | 6 |
| ATOM | 13094 | CB | GLU | F | 510 | 101.523 | 79.088 | 28.822 | 1.00 | 63.47 | 6 |
| ATOM | 13095 | CG | GLU | F | 510 | 102.671 | 78.300 | 29.436 | 1.00 | 65.36 | 6 |
| ATOM | 13096 | CD | GLU | F | 510 | 102.410 | 77.895 | 30.878 | 1.00 | 67.59 | 6 |
| ATOM | 13097 | OE1 | GLU | F | 510 | 101.326 | 77.353 | 31.154 | 1.00 | 69.46 | 8 |
| ATOM | 13098 | OE2 | GLU | F | 510 | 103.288 | 78.108 | 31.742 | 1.00 | 68.15 | 8 |
| ATOM | 13099 | C | GLU | F | 510 | 100.808 | 80.877 | 27.234 | 1.00 | 139.48 | 6 |
| ATOM | 13100 | O | GLU | F | 510 | 100.863 | 80.800 | 26.009 | 1.00 | 141.41 | 8 |
| ATOM | 13101 | N | TRP | F | 511 | 99.784 | 81.394 | 27.902 | 1.00 | 36.04 | 7 |
| ATOM | 13102 | CA | TRP | F | 511 | 98.602 | 81.943 | 27.262 | 1.00 | 35.28 | 6 |
| ATOM | 13103 | CB | TRP | F | 511 | 97.855 | 82.837 | 28.249 | 1.00 | 106.42 | 6 |
| ATOM | 13104 | CG | TRP | F | 511 | 98.740 | 83.855 | 28.899 | 1.00 | 106.87 | 6 |
| ATOM | 13105 | CD2 | TRP | F | 511 | 98.656 | 85.278 | 28.770 | 1.00 | 106.98 | 6 |
| ATOM | 13106 | CE2 | TRP | F | 511 | 99.701 | 85.828 | 29.538 | 1.00 | 107.23 | 6 |
| ATOM | 13107 | CE3 | TRP | F | 511 | 97.801 | 86.140 | 28.081 | 1.00 | 107.48 | 6 |
| ATOM | 13108 | CD1 | TRP | F | 511 | 99.803 | 83.610 | 29.717 | 1.00 | 107.32 | 6 |
| ATOM | 13109 | NE1 | TRP | F | 511 | 100.386 | 84.789 | 30.105 | 1.00 | 107.63 | 7 |
| ATOM | 13110 | CZ2 | TRP | F | 511 | 99.912 | 87.201 | 29.636 | 1.00 | 107.53 | 6 |
| ATOM | 13111 | CZ3 | TRP | F | 511 | 98.015 | 87.506 | 28.181 | 1.00 | 107.86 | 6 |
| ATOM | 13112 | CH2 | TRP | F | 511 | 99.061 | 88.019 | 28.951 | 1.00 | 107.57 | 6 |
| ATOM | 13113 | C | TRP | F | 511 | 98.932 | 82.737 | 26.007 | 1.00 | 34.42 | 6 |
| ATOM | 13114 | O | TRP | F | 511 | 98.894 | 83.962 | 25.989 | 1.00 | 33.08 | 8 |
| ATOM | 13115 | N | MET | F | 512 | 99.247 | 81.991 | 24.964 | 1.00 | 54.37 | 7 |
| ATOM | 13116 | CA | MET | F | 512 | 99.583 | 82.460 | 23.629 | 1.00 | 56.16 | 6 |
| ATOM | 13117 | CB | MET | F | 512 | 100.906 | 83.207 | 23.616 | 1.00 | 102.60 | 6 |
| ATOM | 13118 | CG | MET | F | 512 | 100.767 | 84.612 | 24.147 | 1.00 | 105.97 | 6 |
| ATOM | 13119 | SD | MET | F | 512 | 102.331 | 85.393 | 24.500 | 1.00 | 109.61 | 16 |
| ATOM | 13120 | CE | MET | F | 512 | 103.055 | 84.175 | 25.623 | 1.00 | 110.11 | 6 |
| ATOM | 13121 | C | MET | F | 512 | 99.725 | 81.095 | 23.007 | 1.00 | 55.96 | 6 |
| ATOM | 13122 | O | MET | F | 512 | 100.522 | 80.870 | 22.106 | 1.00 | 55.60 | 8 |
| ATOM | 13123 | N | ILE | F | 513 | 98.921 | 80.196 | 23.578 | 1.00 | 117.50 | 7 |
| ATOM | 13124 | CA | ILE | F | 513 | 98.792 | 78.777 | 23.259 | 1.00 | 117.53 | 6 |
| ATOM | 13125 | CB | ILE | F | 513 | 97.352 | 78.287 | 23.603 | 1.00 | 134.32 | 6 |
| ATOM | 13126 | CG2 | ILE | F | 513 | 97.086 | 76.913 | 23.008 | 1.00 | 134.92 | 6 |
| ATOM | 13127 | CG1 | ILE | F | 513 | 97.171 | 78.232 | 25.120 | 1.00 | 135.24 | 6 |
| ATOM | 13128 | CD1 | ILE | F | 513 | 98.110 | 77.261 | 25.816 | 1.00 | 134.91 | 6 |
| ATOM | 13129 | C | ILE | F | 513 | 99.133 | 78.295 | 21.862 | 1.00 | 117.10 | 6 |
| ATOM | 13130 | O | ILE | F | 513 | 98.947 | 78.998 | 20.869 | 1.00 | 116.57 | 8 |
| ATOM | 13131 | N | LEU | F | 514 | 99.637 | 77.067 | 21.818 | 1.00 | 57.60 | 7 |
| ATOM | 13132 | CA | LEU | F | 514 | 99.994 | 76.404 | 20.581 | 1.00 | 57.13 | 6 |
| ATOM | 13133 | CB | LEU | F | 514 | 101.509 | 76.235 | 20.478 | 1.00 | 186.03 | 6 |
| ATOM | 13134 | CG | LEU | F | 514 | 102.047 | 75.692 | 19.152 | 1.00 | 188.54 | 6 |
| ATOM | 13135 | CD1 | LEU | F | 514 | 101.564 | 76.565 | 18.002 | 1.00 | 189.47 | 6 |
| ATOM | 13136 | CD2 | LEU | F | 514 | 103.569 | 75.661 | 19.192 | 1.00 | 189.74 | 6 |
| ATOM | 13137 | C | LEU | F | 514 | 99.319 | 75.049 | 20.691 | 1.00 | 55.99 | 6 |
| ATOM | 13138 | O | LEU | F | 514 | 99.998 | 74.023 | 20.803 | 1.00 | 55.74 | 8 |
| ATOM | 13139 | N | ALA | F | 515 | 97.979 | 75.076 | 20.691 | 1.00 | 111.70 | 7 |
| ATOM | 13140 | CA | ALA | F | 515 | 97.115 | 73.890 | 20.799 | 1.00 | 110.85 | 6 |
| ATOM | 13141 | CB | ALA | F | 515 | 97.717 | 72.882 | 21.782 | 1.00 | 79.24 | 6 |
| ATOM | 13142 | C | ALA | F | 515 | 95.693 | 74.249 | 21.260 | 1.00 | 109.71 | 6 |
| ATOM | 13143 | O | ALA | F | 515 | 95.379 | 74.100 | 22.442 | 1.00 | 110.12 | 8 |
| ATOM | 13144 | N | ALA | F | 516 | 94.832 | 74.698 | 20.344 | 1.00 | 72.87 | 7 |
| ATOM | 13145 | CA | ALA | F | 516 | 93.449 | 75.070 | 20.707 | 1.00 | 71.59 | 6 |
| ATOM | 13146 | CB | ALA | F | 516 | 92.749 | 75.771 | 19.537 | 1.00 | 13.87 | 6 |
| ATOM | 13147 | C | ALA | F | 516 | 92.608 | 73.885 | 21.169 | 1.00 | 70.92 | 6 |
| ATOM | 13148 | O | ALA | F | 516 | 92.942 | 72.730 | 20.911 | 1.00 | 71.10 | 8 |
| ATOM | 13149 | N | VAL | F | 517 | 91.500 | 74.167 | 21.843 | 1.00 | 66.87 | 7 |
| ATOM | 13150 | CA | VAL | F | 517 | 90.667 | 73.079 | 22.336 | 1.00 | 66.50 | 6 |
| ATOM | 13151 | CB | VAL | F | 517 | 91.093 | 72.689 | 23.766 | 1.00 | 95.37 | 6 |
| ATOM | 13152 | CG1 | VAL | F | 517 | 90.500 | 71.341 | 24.129 | 1.00 | 96.39 | 6 |
| ATOM | 13153 | CG2 | VAL | F | 517 | 92.608 | 72.668 | 23.877 | 1.00 | 94.93 | 6 |
| ATOM | 13154 | C | VAL | F | 517 | 89.148 | 73.321 | 22.348 | 1.00 | 65.39 | 6 |
| ATOM | 13155 | O | VAL | F | 517 | 88.671 | 74.339 | 22.853 | 1.00 | 65.01 | 8 |
| ATOM | 13156 | N | PRO | F | 518 | 88.377 | 72.385 | 21.766 | 1.00 | 77.59 | 7 |
| ATOM | 13157 | CD | PRO | F | 518 | 88.925 | 71.439 | 20.778 | 1.00 | 85.26 | 6 |
| ATOM | 13158 | CA | PRO | F | 518 | 86.915 | 72.405 | 21.675 | 1.00 | 76.43 | 6 |
| ATOM | 13159 | CB | PRO | F | 518 | 86.666 | 71.894 | 20.266 | 1.00 | 84.37 | 6 |
| ATOM | 13160 | CG | PRO | F | 518 | 87.665 | 70.852 | 20.134 | 1.00 | 85.38 | 6 |
| ATOM | 13161 | C | PRO | F | 518 | 86.390 | 71.432 | 22.732 | 1.00 | 75.42 | 6 |
| ATOM | 13162 | O | PRO | F | 518 | 87.074 | 70.450 | 23.058 | 1.00 | 75.98 | 8 |
| ATOM | 13163 | N | VAL | F | 519 | 85.199 | 71.673 | 23.275 | 1.00 | 24.60 | 7 |
| ATOM | 13164 | CA | VAL | F | 519 | 84.701 | 70.741 | 24.284 | 1.00 | 22.89 | 6 |
| ATOM | 13165 | CB | VAL | F | 519 | 83.435 | 71.277 | 25.003 | 1.00 | 39.08 | 6 |
| ATOM | 13166 | CG1 | VAL | F | 519 | 83.111 | 70.391 | 26.209 | 1.00 | 39.89 | 6 |
| ATOM | 13167 | CG2 | VAL | F | 519 | 83.642 | 72.729 | 25.437 | 1.00 | 38.27 | 6 |
| ATOM | 13168 | C | VAL | F | 519 | 84.369 | 69.441 | 23.568 | 1.00 | 21.64 | 6 |
| ATOM | 13169 | O | VAL | F | 519 | 84.515 | 69.349 | 22.352 | 1.00 | 20.81 | 8 |
| ATOM | 13170 | N | LEU | F | 520 | 83.916 | 68.443 | 24.313 | 1.00 | 24.74 | 7 |
| ATOM | 13171 | CA | LEU | F | 520 | 83.568 | 67.149 | 23.736 | 1.00 | 27.17 | 6 |
| ATOM | 13172 | CB | LEU | F | 520 | 83.281 | 66.185 | 24.865 | 1.00 | 54.99 | 6 |
| ATOM | 13173 | CG | LEU | F | 520 | 84.606 | 65.976 | 25.578 | 1.00 | 58.04 | 6 |
| ATOM | 13174 | CD1 | LEU | F | 520 | 84.401 | 65.492 | 27.000 | 1.00 | 58.81 | 6 |
| ATOM | 13175 | CD2 | LEU | F | 520 | 85.417 | 65.024 | 24.731 | 1.00 | 59.39 | 6 |
| ATOM | 13176 | C | LEU | F | 520 | 82.400 | 67.161 | 22.760 | 1.00 | 28.37 | 6 |
| ATOM | 13177 | O | LEU | F | 520 | 81.813 | 68.203 | 22.476 | 1.00 | 28.71 | 8 |
| ATOM | 13178 | N | PRO | F | 521 | 82.067 | 66.002 | 22.193 | 1.00 | 52.35 | 7 |
| ATOM | 13179 | CD | PRO | F | 521 | 82.788 | 64.735 | 21.997 | 1.00 | 122.40 | 6 |
| ATOM | 13180 | CA | PRO | F | 521 | 80.933 | 66.118 | 21.281 | 1.00 | 53.25 | 6 |
| ATOM | 13181 | CB | PRO | F | 521 | 80.907 | 64.761 | 20.567 | 1.00 | 121.72 | 6 |
| ATOM | 13182 | CG | PRO | F | 521 | 81.698 | 63.844 | 21.473 | 1.00 | 123.75 | 6 |
| ATOM | 13183 | C | PRO | F | 521 | 79.616 | 66.460 | 21.986 | 1.00 | 53.55 | 6 |
| ATOM | 13184 | O | PRO | F | 521 | 78.869 | 67.306 | 21.494 | 1.00 | 54.88 | 8 |
| ATOM | 13185 | N | PRO | F | 522 | 79.335 | 65.838 | 23.164 | 1.00 | 59.07 | 7 |
| ATOM | 13186 | CD | PRO | F | 522 | 80.237 | 64.894 | 23.854 | 1.00 | 86.85 | 6 |
| ATOM | 13187 | CA | PRO | F | 522 | 78.118 | 66.037 | 23.976 | 1.00 | 58.98 | 6 |
| ATOM | 13188 | CB | PRO | F | 522 | 78.411 | 65.230 | 25.240 | 1.00 | 86.62 | 6 |
| ATOM | 13189 | CG | PRO | F | 522 | 79.293 | 64.138 | 24.743 | 1.00 | 87.27 | 6 |
| ATOM | 13190 | C | PRO | F | 522 | 77.711 | 67.474 | 24.316 | 1.00 | 58.44 | 6 |
| ATOM | 13191 | O | PRO | F | 522 | 76.787 | 67.687 | 25.092 | 1.00 | 58.06 | 8 |
| ATOM | 13192 | N | ALA | F | 523 | 78.406 | 68.450 | 23.752 | 1.00 | 61.86 | 7 |
| ATOM | 13193 | CA | ALA | F | 523 | 78.095 | 69.843 | 24.006 | 1.00 | 62.64 | 6 |
| ATOM | 13194 | CB | ALA | F | 523 | 79.094 | 70.420 | 25.008 | 1.00 | 67.65 | 6 |
| ATOM | 13195 | C | ALA | F | 523 | 78.183 | 70.574 | 22.667 | 1.00 | 63.46 | 6 |
| ATOM | 13196 | O | ALA | F | 523 | 78.866 | 70.028 | 21.777 | 1.00 | 63.50 | 8 |
| ATOM | 13197 | OXT | ALA | F | 523 | 77.588 | 71.666 | 22.509 | 1.00 | 70.13 | 8 |
| ATOM | 13198 | CB | ALA | G | 536 | 70.546 | 72.838 | 16.847 | 1.00 | 131.36 | 6 |
| ATOM | 13199 | C | ALA | G | 536 | 71.436 | 74.311 | 18.678 | 1.00 | 131.87 | 6 |
| ATOM | 13200 | O | ALA | G | 536 | 71.660 | 75.515 | 18.831 | 1.00 | 131.63 | 8 |
| ATOM | 13201 | N | ALA | G | 536 | 69.463 | 75.007 | 17.368 | 1.00 | 133.22 | 7 |
| ATOM | 13202 | CA | ALA | G | 536 | 70.180 | 73.839 | 17.954 | 1.00 | 132.70 | 6 |
| ATOM | 13203 | N | THR | G | 537 | 72.248 | 73.359 | 19.125 | 1.00 | 95.20 | 7 |
| ATOM | 13204 | CA | THR | G | 537 | 73.488 | 73.673 | 19.819 | 1.00 | 93.78 | 6 |
| ATOM | 13205 | CB | THR | G | 537 | 73.607 | 72.841 | 21.103 | 1.00 | 182.64 | 6 |
| ATOM | 13206 | OG1 | THR | G | 537 | 72.380 | 72.939 | 21.840 | 1.00 | 183.52 | 8 |
| ATOM | 13207 | CG2 | THR | G | 537 | 74.752 | 73.355 | 21.969 | 1.00 | 182.66 | 6 |
| ATOM | 13208 | C | THR | G | 537 | 74.683 | 73.396 | 18.893 | 1.00 | 92.06 | 6 |
| ATOM | 13209 | O | THR | G | 537 | 75.787 | 73.071 | 19.345 | 1.00 | 92.52 | 8 |
| ATOM | 13210 | N | SER | G | 538 | 74.437 | 73.542 | 17.590 | 1.00 | 112.89 | 7 |
| ATOM | 13211 | CA | SER | G | 538 | 75.440 | 73.328 | 16.547 | 1.00 | 110.32 | 6 |
| ATOM | 13212 | CB | SER | G | 538 | 74.779 | 73.283 | 15.160 | 1.00 | 35.57 | 6 |
| ATOM | 13213 | OG | SER | G | 538 | 73.626 | 72.468 | 15.132 | 1.00 | 33.65 | 8 |
| ATOM | 13214 | C | SER | G | 538 | 76.445 | 74.471 | 16.544 | 1.00 | 109.45 | 6 |
| ATOM | 13215 | O | SER | G | 538 | 77.627 | 74.282 | 16.819 | 1.00 | 109.31 | 8 |
| ATOM | 13216 | N | ASP | G | 539 | 75.936 | 75.656 | 16.217 | 1.00 | 117.37 | 7 |
| ATOM | 13217 | CA | ASP | G | 539 | 76.713 | 76.884 | 16.121 | 1.00 | 116.70 | 6 |
| ATOM | 13218 | CB | ASP | G | 539 | 76.184 | 77.922 | 17.114 | 1.00 | 173.02 | 6 |
| ATOM | 13219 | CG | ASP | G | 539 | 76.695 | 79.321 | 16.819 | 1.00 | 175.38 | 6 |
| ATOM | 13220 | OD1 | ASP | G | 539 | 77.894 | 79.581 | 17.045 | 1.00 | 175.93 | 8 |
| ATOM | 13221 | OD2 | ASP | G | 539 | 75.899 | 80.160 | 16.348 | 1.00 | 177.00 | 8 |
| ATOM | 13222 | C | ASP | G | 539 | 78.218 | 76.712 | 16.299 | 1.00 | 114.94 | 6 |
| ATOM | 13223 | O | ASP | G | 539 | 78.943 | 76.520 | 15.323 | 1.00 | 115.19 | 8 |
| ATOM | 13224 | N | LEU | G | 540 | 78.683 | 76.774 | 17.541 | 1.00 | 64.35 | 7 |
| ATOM | 13225 | CA | LEU | G | 540 | 80.111 | 76.649 | 17.842 | 1.00 | 61.65 | 6 |
| ATOM | 13226 | CB | LEU | G | 540 | 80.311 | 76.121 | 19.259 | 1.00 | 82.00 | 6 |
| ATOM | 13227 | CG | LEU | G | 540 | 81.765 | 76.224 | 19.698 | 1.00 | 80.94 | 6 |
| ATOM | 13228 | CD1 | LEU | G | 540 | 81.986 | 77.620 | 20.251 | 1.00 | 81.27 | 6 |
| ATOM | 13229 | CD2 | LEU | G | 540 | 82.081 | 75.173 | 20.741 | 1.00 | 80.77 | 6 |
| ATOM | 13230 | C | LEU | G | 540 | 80.907 | 75.764 | 16.884 | 1.00 | 60.56 | 6 |
| ATOM | 13231 | O | LEU | G | 540 | 81.986 | 76.143 | 16.444 | 1.00 | 60.17 | 8 |
| ATOM | 13232 | N | ASN | G | 541 | 80.376 | 74.582 | 16.586 | 1.00 | 66.76 | 7 |
| ATOM | 13233 | CA | ASN | G | 541 | 81.032 | 73.640 | 15.686 | 1.00 | 65.78 | 6 |
| ATOM | 13234 | CB | ASN | G | 541 | 80.039 | 72.588 | 15.216 | 1.00 | 75.75 | 6 |
| ATOM | 13235 | CG | ASN | G | 541 | 79.680 | 71.614 | 16.306 | 1.00 | 74.86 | 6 |
| ATOM | 13236 | OD1 | ASN | G | 541 | 80.441 | 70.692 | 16.609 | 1.00 | 73.45 | 8 |
| ATOM | 13237 | ND2 | ASN | G | 541 | 78.520 | 71.820 | 16.919 | 1.00 | 75.26 | 7 |
| ATOM | 13238 | C | ASN | G | 541 | 81.629 | 74.337 | 14.485 | 1.00 | 65.33 | 6 |
| ATOM | 13239 | O | ASN | G | 541 | 82.767 | 74.080 | 14.114 | 1.00 | 66.45 | 8 |
| ATOM | 13240 | N | ASP | G | 542 | 80.852 | 75.209 | 13.864 | 1.00 | 41.08 | 7 |
| ATOM | 13241 | CA | ASP | G | 542 | 81.360 | 75.950 | 12.726 | 1.00 | 41.48 | 6 |
| ATOM | 13242 | CB | ASP | G | 542 | 80.364 | 77.019 | 12.277 | 1.00 | 94.27 | 6 |
| ATOM | 13243 | CG | ASP | G | 542 | 79.244 | 76.449 | 11.452 | 1.00 | 95.35 | 6 |
| ATOM | 13244 | OD1 | ASP | G | 542 | 79.533 | 75.643 | 10.543 | 1.00 | 94.76 | 8 |
| ATOM | 13245 | OD2 | ASP | G | 542 | 78.080 | 76.817 | 11.703 | 1.00 | 96.59 | 8 |
| ATOM | 13246 | C | ASP | G | 542 | 82.693 | 76.612 | 13.087 | 1.00 | 41.73 | 6 |
| ATOM | 13247 | O | ASP | G | 542 | 83.748 | 76.150 | 12.658 | 1.00 | 41.89 | 8 |
| ATOM | 13248 | N | LEU | G | 543 | 82.654 | 77.685 | 13.874 | 1.00 | 77.19 | 7 |
| ATOM | 13249 | CA | LEU | G | 543 | 83.881 | 78.371 | 14.256 | 1.00 | 77.54 | 6 |
| ATOM | 13250 | CB | LEU | G | 543 | 83.709 | 79.050 | 15.620 | 1.00 | 134.25 | 6 |
| ATOM | 13251 | CG | LEU | G | 543 | 82.765 | 80.260 | 15.677 | 1.00 | 135.12 | 6 |
| ATOM | 13252 | CD1 | LEU | G | 543 | 82.767 | 80.850 | 17.074 | 1.00 | 135.70 | 6 |
| ATOM | 13253 | CD2 | LEU | G | 543 | 83.213 | 81.310 | 14.678 | 1.00 | 135.15 | 6 |
| ATOM | 13254 | C | LEU | G | 543 | 85.092 | 77.429 | 14.276 | 1.00 | 77.57 | 6 |
| ATOM | 13255 | O | LEU | G | 543 | 86.180 | 77.807 | 13.841 | 1.00 | 76.88 | 8 |
| ATOM | 13256 | N | TYR | G | 544 | 84.897 | 76.202 | 14.764 | 1.00 | 68.61 | 7 |
| ATOM | 13257 | CA | TYR | G | 544 | 85.975 | 75.215 | 14.813 | 1.00 | 69.05 | 6 |
| ATOM | 13258 | CB | TYR | G | 544 | 85.582 | 74.025 | 15.690 | 1.00 | 71.08 | 6 |
| ATOM | 13259 | CG | TYR | G | 544 | 85.868 | 74.295 | 17.143 | 1.00 | 71.98 | 6 |
| ATOM | 13260 | CD1 | TYR | G | 544 | 84.989 | 73.902 | 18.141 | 1.00 | 72.88 | 6 |
| ATOM | 13261 | CE1 | TYR | G | 544 | 85.233 | 74.220 | 19.474 | 1.00 | 73.58 | 6 |
| ATOM | 13262 | CD2 | TYR | G | 544 | 87.005 | 75.003 | 17.515 | 1.00 | 72.46 | 6 |
| ATOM | 13263 | CE2 | TYR | G | 544 | 87.262 | 75.318 | 18.838 | 1.00 | 72.62 | 6 |
| ATOM | 13264 | CZ | TYR | G | 544 | 86.378 | 74.927 | 19.810 | 1.00 | 73.30 | 6 |
| ATOM | 13265 | OH | TYR | G | 544 | 86.658 | 75.226 | 21.119 | 1.00 | 74.86 | 8 |
| ATOM | 13266 | C | TYR | G | 544 | 86.398 | 74.746 | 13.430 | 1.00 | 69.44 | 6 |
| ATOM | 13267 | O | TYR | G | 544 | 87.586 | 74.615 | 13.157 | 1.00 | 70.07 | 8 |
| ATOM | 13268 | N | ARG | G | 545 | 85.439 | 74.487 | 12.551 | 1.00 | 56.19 | 7 |
| ATOM | 13269 | CA | ARG | G | 545 | 85.795 | 74.086 | 11.202 | 1.00 | 56.68 | 6 |
| ATOM | 13270 | CB | ARG | G | 545 | 84.541 | 73.820 | 10.352 | 1.00 | 106.98 | 6 |
| ATOM | 13271 | CG | ARG | G | 545 | 83.823 | 75.091 | 9.908 | 1.00 | 108.55 | 6 |
| ATOM | 13272 | CD | ARG | G | 545 | 82.671 | 74.871 | 8.937 | 1.00 | 109.66 | 6 |
| ATOM | 13273 | NE | ARG | G | 545 | 82.266 | 76.145 | 8.344 | 1.00 | 109.87 | 7 |
| ATOM | 13274 | CZ | ARG | G | 545 | 81.233 | 76.300 | 7.526 | 1.00 | 110.78 | 6 |
| ATOM | 13275 | NH1 | ARG | G | 545 | 80.482 | 75.258 | 7.195 | 1.00 | 111.29 | 7 |
| ATOM | 13276 | NH2 | ARG | G | 545 | 80.958 | 77.498 | 7.029 | 1.00 | 111.30 | 7 |
| ATOM | 13277 | C | ARG | G | 545 | 86.565 | 75.298 | 10.647 | 1.00 | 56.31 | 6 |
| ATOM | 13278 | O | ARG | G | 545 | 87.674 | 75.155 | 10.134 | 1.00 | 56.35 | 8 |
| ATOM | 13279 | N | ARG | G | 546 | 85.978 | 76.491 | 10.783 | 1.00 | 67.98 | 7 |
| ATOM | 13280 | CA | ARG | G | 546 | 86.588 | 77.732 | 10.304 | 1.00 | 68.08 | 6 |
| ATOM | 13281 | CB | ARG | G | 546 | 85.836 | 78.939 | 10.847 | 1.00 | 83.15 | 6 |
| ATOM | 13282 | CG | ARG | G | 546 | 84.614 | 79.331 | 10.071 | 1.00 | 84.94 | 6 |
| ATOM | 13283 | CD | ARG | G | 546 | 84.037 | 80.602 | 10.665 | 1.00 | 87.67 | 6 |
| ATOM | 13284 | NE | ARG | G | 546 | 85.082 | 81.578 | 10.987 | 1.00 | 90.57 | 7 |
| ATOM | 13285 | CZ | ARG | G | 546 | 84.864 | 82.775 | 11.534 | 1.00 | 91.12 | 6 |
| ATOM | 13286 | NH1 | ARG | G | 546 | 83.628 | 83.168 | 11.831 | 1.00 | 91.08 | 7 |
| ATOM | 13287 | NH2 | ARG | G | 546 | 85.886 | 83.584 | 11.791 | 1.00 | 91.46 | 7 |
| ATOM | 13288 | C | ARG | G | 546 | 88.031 | 77.820 | 10.755 | 1.00 | 67.70 | 6 |
| ATOM | 13289 | O | ARG | G | 546 | 88.828 | 78.556 | 10.174 | 1.00 | 67.44 | 8 |
| ATOM | 13290 | N | LEU | G | 547 | 88.341 | 77.073 | 11.812 | 1.00 | 84.34 | 7 |
| ATOM | 13291 | CA | LEU | G | 547 | 89.677 | 77.011 | 12.393 | 1.00 | 83.70 | 6 |
| ATOM | 13292 | CB | LEU | G | 547 | 89.580 | 76.874 | 13.913 | 1.00 | 76.64 | 6 |
| ATOM | 13293 | CG | LEU | G | 547 | 90.889 | 76.614 | 14.661 | 1.00 | 76.80 | 6 |
| ATOM | 13294 | CD1 | LEU | G | 547 | 91.608 | 77.924 | 14.896 | 1.00 | 77.40 | 6 |
| ATOM | 13295 | CD2 | LEU | G | 547 | 90.603 | 75.952 | 15.985 | 1.00 | 76.94 | 6 |
| ATOM | 13296 | C | LEU | G | 547 | 90.400 | 75.794 | 11.841 | 1.00 | 83.08 | 6 |
| ATOM | 13297 | O | LEU | G | 547 | 91.332 | 75.898 | 11.045 | 1.00 | 83.25 | 8 |
| ATOM | 13298 | N | ILE | G | 548 | 89.950 | 74.631 | 12.287 | 1.00 | 54.14 | 7 |
| ATOM | 13299 | CA | ILE | G | 548 | 90.523 | 73.363 | 11.868 | 1.00 | 53.72 | 6 |
| ATOM | 13300 | CB | ILE | G | 548 | 89.540 | 72.202 | 12.135 | 1.00 | 76.27 | 6 |
| ATOM | 13301 | CG2 | ILE | G | 548 | 90.237 | 70.884 | 11.902 | 1.00 | 75.88 | 6 |
| ATOM | 13302 | CG1 | ILE | G | 548 | 88.996 | 72.282 | 13.569 | 1.00 | 75.82 | 6 |
| ATOM | 13303 | CD1 | ILE | G | 548 | 90.056 | 72.520 | 14.639 | 1.00 | 75.63 | 6 |
| ATOM | 13304 | C | ILE | G | 548 | 90.911 | 73.352 | 10.391 | 1.00 | 53.09 | 6 |
| ATOM | 13305 | O | ILE | G | 548 | 91.976 | 72.862 | 10.039 | 1.00 | 52.98 | 8 |
| ATOM | 13306 | N | ASN | G | 549 | 90.050 | 73.892 | 9.533 | 1.00 | 131.40 | 7 |
| ATOM | 13307 | CA | ASN | G | 549 | 90.325 | 73.931 | 8.096 | 1.00 | 131.28 | 6 |
| ATOM | 13308 | CB | ASN | G | 549 | 89.021 | 74.162 | 7.300 | 1.00 | 74.52 | 6 |
| ATOM | 13309 | CG | ASN | G | 549 | 88.071 | 72.951 | 7.320 | 1.00 | 73.67 | 6 |
| ATOM | 13310 | OD1 | ASN | G | 549 | 86.958 | 73.014 | 6.783 | 1.00 | 72.98 | 8 |
| ATOM | 13311 | ND2 | ASN | G | 549 | 88.506 | 71.856 | 7.932 | 1.00 | 72.43 | 7 |
| ATOM | 13312 | C | ASN | G | 549 | 91.348 | 75.024 | 7.746 | 1.00 | 131.21 | 6 |
| ATOM | 13313 | O | ASN | G | 549 | 92.219 | 74.820 | 6.897 | 1.00 | 131.77 | 8 |
| ATOM | 13314 | N | ARG | G | 550 | 91.241 | 76.178 | 8.405 | 1.00 | 46.50 | 7 |
| ATOM | 13315 | CA | ARG | G | 550 | 92.141 | 77.307 | 8.166 | 1.00 | 46.38 | 6 |
| ATOM | 13316 | CB | ARG | G | 550 | 91.635 | 78.564 | 8.871 | 1.00 | 133.91 | 6 |
| ATOM | 13317 | CG | ARG | G | 550 | 90.706 | 79.418 | 8.036 | 1.00 | 135.97 | 6 |
| ATOM | 13318 | CD | ARG | G | 550 | 91.446 | 80.080 | 6.888 | 1.00 | 137.05 | 6 |
| ATOM | 13319 | NE | ARG | G | 550 | 90.559 | 80.933 | 6.102 | 1.00 | 138.25 | 7 |
| ATOM | 13320 | CZ | ARG | G | 550 | 90.940 | 81.628 | 5.037 | 1.00 | 138.54 | 6 |
| ATOM | 13321 | NH1 | ARG | G | 550 | 92.199 | 81.577 | 4.622 | 1.00 | 139.13 | 7 |
| ATOM | 13322 | NH2 | ARG | G | 550 | 90.058 | 82.373 | 4.382 | 1.00 | 138.59 | 7 |
| ATOM | 13323 | C | ARG | G | 550 | 93.533 | 77.004 | 8.655 | 1.00 | 45.82 | 6 |
| ATOM | 13324 | O | ARG | G | 550 | 94.505 | 77.441 | 8.054 | 1.00 | 45.25 | 8 |
| ATOM | 13325 | N | ASN | G | 551 | 93.622 | 76.266 | 9.758 | 1.00 | 60.10 | 7 |
| ATOM | 13326 | CA | ASN | G | 551 | 94.910 | 75.894 | 10.327 | 1.00 | 61.10 | 6 |
| ATOM | 13327 | CB | ASN | G | 551 | 94.710 | 75.198 | 11.680 | 1.00 | 37.67 | 6 |
| ATOM | 13328 | CG | ASN | G | 551 | 96.031 | 74.889 | 12.395 | 1.00 | 36.35 | 6 |
| ATOM | 13329 | OD1 | ASN | G | 551 | 96.508 | 73.748 | 12.391 | 1.00 | 35.15 | 8 |
| ATOM | 13330 | ND2 | ASN | G | 551 | 96.624 | 75.908 | 13.013 | 1.00 | 35.68 | 7 |
| ATOM | 13331 | C | ASN | G | 551 | 95.614 | 74.966 | 9.339 | 1.00 | 63.12 | 6 |
| ATOM | 13332 | O | ASN | G | 551 | 96.783 | 74.615 | 9.516 | 1.00 | 63.81 | 8 |
| ATOM | 13333 | N | ASN | G | 552 | 94.891 | 74.579 | 8.290 | 1.00 | 69.42 | 7 |
| ATOM | 13334 | CA | ASN | G | 552 | 95.436 | 73.705 | 7.256 | 1.00 | 70.97 | 6 |
| ATOM | 13335 | CB | ASN | G | 552 | 94.442 | 72.591 | 6.906 | 1.00 | 117.93 | 6 |
| ATOM | 13336 | CG | ASN | G | 552 | 94.372 | 71.515 | 7.976 | 1.00 | 118.17 | 6 |
| ATOM | 13337 | OD1 | ASN | G | 552 | 95.388 | 70.926 | 8.352 | 1.00 | 118.04 | 8 |
| ATOM | 13338 | ND2 | ASN | G | 552 | 93.169 | 71.248 | 8.467 | 1.00 | 118.44 | 7 |
| ATOM | 13339 | C | ASN | G | 552 | 95.805 | 74.486 | 6.003 | 1.00 | 71.92 | 6 |
| ATOM | 13340 | O | ASN | G | 552 | 96.810 | 74.186 | 5.368 | 1.00 | 71.09 | 8 |
| ATOM | 13341 | N | ARG | G | 553 | 94.998 | 75.484 | 5.642 | 1.00 | 115.12 | 7 |
| ATOM | 13342 | CA | ARG | G | 553 | 95.306 | 76.297 | 4.467 | 1.00 | 116.95 | 6 |
| ATOM | 13343 | CB | ARG | G | 553 | 94.268 | 77.404 | 4.259 | 1.00 | 201.34 | 6 |
| ATOM | 13344 | CG | ARG | G | 553 | 93.017 | 76.966 | 3.510 | 1.00 | 204.50 | 6 |
| ATOM | 13345 | CD | ARG | G | 553 | 92.387 | 78.151 | 2.778 | 1.00 | 207.23 | 6 |
| ATOM | 13346 | NE | ARG | G | 553 | 91.118 | 77.808 | 2.136 | 1.00 | 208.87 | 7 |
| ATOM | 13347 | CZ | ARG | G | 553 | 90.443 | 78.621 | 1.327 | 1.00 | 208.87 | 6 |
| ATOM | 13348 | NH1 | ARG | G | 553 | 90.916 | 79.831 | 1.051 | 1.00 | 208.87 | 7 |
| ATOM | 13349 | NH2 | ARG | G | 553 | 89.291 | 78.229 | 0.797 | 1.00 | 208.87 | 7 |
| ATOM | 13350 | C | ARG | G | 553 | 96.686 | 76.922 | 4.657 | 1.00 | 116.81 | 6 |
| ATOM | 13351 | O | ARG | G | 553 | 97.207 | 77.597 | 3.767 | 1.00 | 116.82 | 8 |
| ATOM | 13352 | N | LEU | G | 554 | 97.259 | 76.702 | 5.839 | 1.00 | 77.44 | 7 |
| ATOM | 13353 | CA | LEU | G | 554 | 98.590 | 77.188 | 6.173 | 1.00 | 76.30 | 6 |
| ATOM | 13354 | CB | LEU | G | 554 | 98.541 | 78.218 | 7.299 | 1.00 | 53.28 | 6 |
| ATOM | 13355 | CG | LEU | G | 554 | 97.775 | 79.499 | 6.949 | 1.00 | 51.92 | 6 |
| ATOM | 13356 | CD1 | LEU | G | 554 | 98.280 | 80.623 | 7.847 | 1.00 | 51.49 | 6 |
| ATOM | 13357 | CD2 | LEU | G | 554 | 97.969 | 79.863 | 5.470 | 1.00 | 50.96 | 6 |
| ATOM | 13358 | C | LEU | G | 554 | 99.375 | 75.976 | 6.607 | 1.00 | 76.50 | 6 |
| ATOM | 13359 | O | LEU | G | 554 | 100.577 | 76.036 | 6.812 | 1.00 | 76.74 | 8 |
| ATOM | 13360 | N | LYS | G | 555 | 98.665 | 74.868 | 6.757 | 1.00 | 106.94 | 7 |
| ATOM | 13361 | CA | LYS | G | 555 | 99.287 | 73.615 | 7.128 | 1.00 | 108.58 | 6 |
| ATOM | 13362 | CB | LYS | G | 555 | 98.342 | 72.804 | 8.016 | 1.00 | 80.09 | 6 |
| ATOM | 13363 | CG | LYS | G | 555 | 98.939 | 71.538 | 8.586 | 1.00 | 79.42 | 6 |
| ATOM | 13364 | CD | LYS | G | 555 | 100.243 | 71.801 | 9.308 | 1.00 | 78.74 | 6 |
| ATOM | 13365 | CE | LYS | G | 555 | 100.636 | 70.601 | 10.148 | 1.00 | 78.94 | 6 |
| ATOM | 13366 | NZ | LYS | G | 555 | 99.639 | 70.349 | 11.234 | 1.00 | 77.84 | 7 |
| ATOM | 13367 | C | LYS | G | 555 | 99.545 | 72.906 | 5.797 | 1.00 | 110.46 | 6 |
| ATOM | 13368 | O | LYS | G | 555 | 100.151 | 71.832 | 5.747 | 1.00 | 111.14 | 8 |
| ATOM | 13369 | N | LYS | G | 556 | 99.075 | 73.539 | 4.721 | 1.00 | 135.11 | 7 |
| ATOM | 13370 | CA | LYS | G | 556 | 99.233 | 73.037 | 3.357 | 1.00 | 135.51 | 6 |
| ATOM | 13371 | CB | LYS | G | 556 | 97.868 | 72.837 | 2.687 | 1.00 | 142.13 | 6 |
| ATOM | 13372 | CG | LYS | G | 556 | 97.028 | 71.713 | 3.290 | 1.00 | 142.48 | 6 |
| ATOM | 13373 | CD | LYS | G | 556 | 95.623 | 71.672 | 2.688 | 1.00 | 142.59 | 6 |
| ATOM | 13374 | CE | LYS | G | 556 | 94.741 | 70.623 | 3.364 | 1.00 | 142.54 | 6 |
| ATOM | 13375 | NZ | LYS | G | 556 | 93.332 | 70.658 | 2.864 | 1.00 | 142.10 | 7 |
| ATOM | 13376 | C | LYS | G | 556 | 100.049 | 74.048 | 2.560 | 1.00 | 135.82 | 6 |
| ATOM | 13377 | O | LYS | G | 556 | 100.940 | 73.674 | 1.801 | 1.00 | 136.26 | 8 |
| ATOM | 13378 | N | LEU | G | 557 | 99.737 | 75.330 | 2.731 | 1.00 | 76.16 | 7 |
| ATOM | 13379 | CA | LEU | G | 557 | 100.470 | 76.384 | 2.040 | 1.00 | 77.22 | 6 |
| ATOM | 13380 | CB | LEU | G | 557 | 99.564 | 77.602 | 1.785 | 1.00 | 59.29 | 6 |
| ATOM | 13381 | CG | LEU | G | 557 | 100.192 | 78.906 | 1.253 | 1.00 | 58.84 | 6 |
| ATOM | 13382 | CD1 | LEU | G | 557 | 101.043 | 78.626 | 0.034 | 1.00 | 57.92 | 6 |
| ATOM | 13383 | CD2 | LEU | G | 557 | 99.105 | 79.909 | 0.923 | 1.00 | 58.61 | 6 |
| ATOM | 13384 | C | LEU | G | 557 | 101.679 | 76.772 | 2.893 | 1.00 | 78.63 | 6 |
| ATOM | 13385 | O | LEU | G | 557 | 101.893 | 77.940 | 3.218 | 1.00 | 79.24 | 8 |
| ATOM | 13386 | N | LEU | G | 558 | 102.464 | 75.769 | 3.265 | 1.00 | 169.46 | 7 |
| ATOM | 13387 | CA | LEU | G | 558 | 103.656 | 75.990 | 4.070 | 1.00 | 171.40 | 6 |
| ATOM | 13388 | CB | LEU | G | 558 | 103.289 | 76.168 | 5.547 | 1.00 | 136.45 | 6 |
| ATOM | 13389 | CG | LEU | G | 558 | 103.083 | 77.619 | 5.980 | 1.00 | 135.88 | 6 |
| ATOM | 13390 | CD1 | LEU | G | 558 | 102.785 | 77.695 | 7.467 | 1.00 | 136.62 | 6 |
| ATOM | 13391 | CD2 | LEU | G | 558 | 104.343 | 78.398 | 5.665 | 1.00 | 134.39 | 6 |
| ATOM | 13392 | C | LEU | G | 558 | 104.650 | 74.852 | 3.907 | 1.00 | 172.39 | 6 |
| ATOM | 13393 | O | LEU | G | 558 | 105.807 | 75.084 | 3.554 | 1.00 | 173.07 | 8 |
| ATOM | 13394 | N | ALA | G | 559 | 104.204 | 73.624 | 4.161 | 1.00 | 188.38 | 7 |
| ATOM | 13395 | CA | ALA | G | 559 | 105.076 | 72.467 | 4.008 | 1.00 | 189.87 | 6 |
| ATOM | 13396 | CB | ALA | G | 559 | 104.358 | 71.196 | 4.429 | 1.00 | 39.70 | 6 |
| ATOM | 13397 | C | ALA | G | 559 | 105.446 | 72.403 | 2.534 | 1.00 | 190.61 | 6 |
| ATOM | 13398 | O | ALA | G | 559 | 106.343 | 71.660 | 2.133 | 1.00 | 191.14 | 8 |
| ATOM | 13399 | N | GLN | G | 560 | 104.733 | 73.196 | 1.738 | 1.00 | 128.76 | 7 |
| ATOM | 13400 | CA | GLN | G | 560 | 104.964 | 73.284 | 0.302 | 1.00 | 128.89 | 6 |
| ATOM | 13401 | CB | GLN | G | 560 | 103.797 | 72.657 | −0.469 | 1.00 | 121.79 | 6 |
| ATOM | 13402 | CG | GLN | G | 560 | 103.586 | 71.179 | −0.175 | 1.00 | 122.17 | 6 |
| ATOM | 13403 | CD | GLN | G | 560 | 102.593 | 70.532 | −1.118 | 1.00 | 122.43 | 6 |
| ATOM | 13404 | OE1 | GLN | G | 560 | 101.448 | 70.964 | −1.221 | 1.00 | 123.35 | 8 |
| ATOM | 13405 | NE2 | GLN | G | 560 | 103.028 | 69.487 | −1.810 | 1.00 | 122.60 | 7 |
| ATOM | 13406 | C | GLN | G | 560 | 105.134 | 74.746 | −0.110 | 1.00 | 128.84 | 6 |
| ATOM | 13407 | O | GLN | G | 560 | 104.984 | 75.654 | 0.714 | 1.00 | 129.24 | 8 |
| ATOM | 13408 | N | GLY | G | 561 | 105.451 | 74.957 | −1.385 | 1.00 | 190.26 | 7 |
| ATOM | 13409 | CA | GLY | G | 561 | 105.647 | 76.296 | −1.916 | 1.00 | 190.02 | 6 |
| ATOM | 13410 | C | GLY | G | 561 | 104.978 | 77.392 | −1.114 | 1.00 | 189.66 | 6 |
| ATOM | 13411 | O | GLY | G | 561 | 103.755 | 77.532 | −1.131 | 1.00 | 190.29 | 8 |
| ATOM | 13412 | N | ALA | G | 562 | 105.787 | 78.174 | −0.411 | 1.00 | 119.23 | 7 |
| ATOM | 13413 | CA | ALA | G | 562 | 105.265 | 79.251 | 0.412 | 1.00 | 117.96 | 6 |
| ATOM | 13414 | CB | ALA | G | 562 | 105.760 | 79.081 | 1.844 | 1.00 | 182.56 | 6 |
| ATOM | 13415 | C | ALA | G | 562 | 105.647 | 80.634 | −0.110 | 1.00 | 117.28 | 6 |
| ATOM | 13416 | O | ALA | G | 562 | 106.755 | 81.112 | 0.132 | 1.00 | 117.09 | 8 |
| ATOM | 13417 | N | PRO | G | 563 | 104.730 | 81.294 | −0.836 | 1.00 | 168.71 | 7 |
| ATOM | 13418 | CD | PRO | G | 563 | 103.438 | 80.757 | −1.294 | 1.00 | 81.97 | 6 |
| ATOM | 13419 | CA | PRO | G | 563 | 104.956 | 82.630 | −1.396 | 1.00 | 168.36 | 6 |
| ATOM | 13420 | CB | PRO | G | 563 | 103.751 | 82.823 | −2.315 | 1.00 | 82.67 | 6 |
| ATOM | 13421 | CG | PRO | G | 563 | 102.696 | 82.014 | −1.664 | 1.00 | 81.98 | 6 |
| ATOM | 13422 | C | PRO | G | 563 | 105.061 | 83.727 | −0.340 | 1.00 | 167.47 | 6 |
| ATOM | 13423 | O | PRO | G | 563 | 104.831 | 84.899 | −0.632 | 1.00 | 167.60 | 8 |
| ATOM | 13424 | N | GLU | G | 564 | 105.412 | 83.338 | 0.882 | 1.00 | 84.57 | 7 |
| ATOM | 13425 | CA | GLU | G | 564 | 105.551 | 84.279 | 1.993 | 1.00 | 82.85 | 6 |
| ATOM | 13426 | CB | GLU | G | 564 | 106.877 | 85.042 | 1.902 | 1.00 | 131.49 | 6 |
| ATOM | 13427 | CG | GLU | G | 564 | 107.064 | 86.063 | 3.023 | 1.00 | 130.94 | 6 |
| ATOM | 13428 | CD | GLU | G | 564 | 108.468 | 86.634 | 3.082 | 1.00 | 131.41 | 6 |
| ATOM | 13429 | OE1 | GLU | G | 564 | 108.935 | 87.177 | 2.059 | 1.00 | 131.51 | 8 |
| ATOM | 13430 | OE2 | GLU | G | 564 | 109.102 | 86.542 | 4.155 | 1.00 | 131.61 | 8 |
| ATOM | 13431 | C | GLU | G | 564 | 104.402 | 85.276 | 2.069 | 1.00 | 81.57 | 6 |
| ATOM | 13432 | O | GLU | G | 564 | 103.463 | 85.077 | 2.832 | 1.00 | 81.23 | 8 |
| ATOM | 13433 | N | ILE | G | 565 | 104.485 | 86.351 | 1.288 | 1.00 | 64.47 | 7 |
| ATOM | 13434 | CA | ILE | G | 565 | 103.437 | 87.359 | 1.277 | 1.00 | 63.84 | 6 |
| ATOM | 13435 | CB | ILE | G | 565 | 103.437 | 88.160 | −0.035 | 1.00 | 111.42 | 6 |
| ATOM | 13436 | CG2 | ILE | G | 565 | 102.234 | 89.085 | −0.081 | 1.00 | 111.05 | 6 |
| ATOM | 13437 | CG1 | ILE | G | 565 | 104.728 | 88.969 | −0.143 | 1.00 | 111.53 | 6 |
| ATOM | 13438 | CD1 | ILE | G | 565 | 104.949 | 89.930 | 1.017 | 1.00 | 111.89 | 6 |
| ATOM | 13439 | C | ILE | G | 565 | 102.087 | 86.682 | 1.449 | 1.00 | 64.00 | 6 |
| ATOM | 13440 | O | ILE | G | 565 | 101.221 | 87.189 | 2.151 | 1.00 | 63.89 | 8 |
| ATOM | 13441 | N | ILE | G | 566 | 101.916 | 85.526 | 0.819 | 1.00 | 179.33 | 7 |
| ATOM | 13442 | CA | ILE | G | 566 | 100.668 | 84.792 | 0.940 | 1.00 | 180.03 | 6 |
| ATOM | 13443 | CB | ILE | G | 566 | 100.147 | 84.363 | −0.450 | 1.00 | 75.12 | 6 |
| ATOM | 13444 | CG2 | ILE | G | 566 | 98.889 | 83.512 | −0.315 | 1.00 | 75.35 | 6 |
| ATOM | 13445 | CG1 | ILE | G | 566 | 99.797 | 85.609 | −1.266 | 1.00 | 74.49 | 6 |
| ATOM | 13446 | CD1 | ILE | G | 566 | 98.646 | 86.422 | −0.679 | 1.00 | 73.42 | 6 |
| ATOM | 13447 | C | ILE | G | 566 | 100.833 | 83.587 | 1.870 | 1.00 | 180.68 | 6 |
| ATOM | 13448 | O | ILE | G | 566 | 100.608 | 82.437 | 1.498 | 1.00 | 181.17 | 8 |
| ATOM | 13449 | N | ILE | G | 567 | 101.247 | 83.895 | 3.093 | 1.00 | 98.19 | 7 |
| ATOM | 13450 | CA | ILE | G | 567 | 101.444 | 82.939 | 4.179 | 1.00 | 98.23 | 6 |
| ATOM | 13451 | CB | ILE | G | 567 | 102.750 | 82.063 | 4.003 | 1.00 | 43.04 | 6 |
| ATOM | 13452 | CG2 | ILE | G | 567 | 103.968 | 82.704 | 4.664 | 1.00 | 42.72 | 6 |
| ATOM | 13453 | CG1 | ILE | G | 567 | 102.533 | 80.692 | 4.646 | 1.00 | 41.40 | 6 |
| ATOM | 13454 | CD1 | ILE | G | 567 | 102.184 | 80.738 | 6.100 | 1.00 | 40.12 | 6 |
| ATOM | 13455 | C | ILE | G | 567 | 101.560 | 83.890 | 5.365 | 1.00 | 99.77 | 6 |
| ATOM | 13456 | O | ILE | G | 567 | 101.225 | 83.556 | 6.501 | 1.00 | 99.67 | 8 |
| ATOM | 13457 | N | ARG | G | 568 | 102.019 | 85.101 | 5.059 | 1.00 | 204.23 | 7 |
| ATOM | 13458 | CA | ARG | G | 568 | 102.168 | 86.161 | 6.046 | 1.00 | 205.67 | 6 |
| ATOM | 13459 | CB | ARG | G | 568 | 103.064 | 87.285 | 5.509 | 1.00 | 199.88 | 6 |
| ATOM | 13460 | CG | ARG | G | 568 | 104.496 | 86.884 | 5.172 | 1.00 | 201.87 | 6 |
| ATOM | 13461 | CD | ARG | G | 568 | 105.309 | 86.559 | 6.417 | 1.00 | 202.96 | 6 |
| ATOM | 13462 | NE | ARG | G | 568 | 106.711 | 86.302 | 6.095 | 1.00 | 204.01 | 7 |
| ATOM | 13463 | CZ | ARG | G | 568 | 107.638 | 85.983 | 6.993 | 1.00 | 204.35 | 6 |
| ATOM | 13464 | NH1 | ARG | G | 568 | 107.316 | 85.880 | 8.276 | 1.00 | 205.26 | 7 |
| ATOM | 13465 | NH2 | ARG | G | 568 | 108.889 | 85.769 | 6.610 | 1.00 | 203.67 | 7 |
| ATOM | 13466 | C | ARG | G | 568 | 100.771 | 86.716 | 6.287 | 1.00 | 205.52 | 6 |
| ATOM | 13467 | O | ARG | G | 568 | 100.192 | 86.521 | 7.356 | 1.00 | 205.56 | 8 |
| ATOM | 13468 | N | ASN | G | 569 | 100.236 | 87.401 | 5.277 | 1.00 | 172.62 | 7 |
| ATOM | 13469 | CA | ASN | G | 569 | 98.904 | 87.986 | 5.370 | 1.00 | 172.19 | 6 |
| ATOM | 13470 | CB | ASN | G | 569 | 98.698 | 89.053 | 4.284 | 1.00 | 144.53 | 6 |
| ATOM | 13471 | CG | ASN | G | 569 | 98.178 | 88.475 | 2.983 | 1.00 | 144.70 | 6 |
| ATOM | 13472 | OD1 | ASN | G | 569 | 98.792 | 87.589 | 2.394 | 1.00 | 143.89 | 8 |
| ATOM | 13473 | ND2 | ASN | G | 569 | 97.036 | 88.978 | 2.528 | 1.00 | 144.81 | 7 |
| ATOM | 13474 | C | ASN | G | 569 | 97.859 | 86.885 | 5.228 | 1.00 | 171.70 | 6 |
| ATOM | 13475 | O | ASN | G | 569 | 96.660 | 87.155 | 5.133 | 1.00 | 172.20 | 8 |
| ATOM | 13476 | N | GLU | G | 570 | 98.326 | 85.640 | 5.207 | 1.00 | 59.76 | 7 |
| ATOM | 13477 | CA | GLU | G | 570 | 97.435 | 84.490 | 5.100 | 1.00 | 58.89 | 6 |
| ATOM | 13478 | CB | GLU | G | 570 | 98.068 | 83.409 | 4.220 | 1.00 | 155.66 | 6 |
| ATOM | 13479 | CG | GLU | G | 570 | 97.969 | 83.685 | 2.732 | 1.00 | 156.86 | 6 |
| ATOM | 13480 | CD | GLU | G | 570 | 96.643 | 83.254 | 2.142 | 1.00 | 157.48 | 6 |
| ATOM | 13481 | OE1 | GLU | G | 570 | 95.591 | 83.672 | 2.667 | 1.00 | 158.14 | 8 |
| ATOM | 13482 | OE2 | GLU | G | 570 | 96.651 | 82.496 | 1.150 | 1.00 | 157.84 | 8 |
| ATOM | 13483 | C | GLU | G | 570 | 97.191 | 83.944 | 6.497 | 1.00 | 57.95 | 6 |
| ATOM | 13484 | O | GLU | G | 570 | 96.143 | 83.369 | 6.777 | 1.00 | 57.43 | 8 |
| ATOM | 13485 | N | LYS | G | 571 | 98.181 | 84.150 | 7.364 | 1.00 | 91.40 | 7 |
| ATOM | 13486 | CA | LYS | G | 571 | 98.144 | 83.700 | 8.755 | 1.00 | 90.81 | 6 |
| ATOM | 13487 | CB | LYS | G | 571 | 99.574 | 83.532 | 9.271 | 1.00 | 152.58 | 6 |
| ATOM | 13488 | CG | LYS | G | 571 | 99.697 | 82.781 | 10.582 | 1.00 | 153.34 | 6 |
| ATOM | 13489 | CD | LYS | G | 571 | 101.152 | 82.416 | 10.836 | 1.00 | 154.51 | 6 |
| ATOM | 13490 | CE | LYS | G | 571 | 101.320 | 81.590 | 12.097 | 1.00 | 154.99 | 6 |
| ATOM | 13491 | NZ | LYS | G | 571 | 101.051 | 82.367 | 13.334 | 1.00 | 155.02 | 7 |
| ATOM | 13492 | C | LYS | G | 571 | 97.378 | 84.684 | 9.640 | 1.00 | 89.48 | 6 |
| ATOM | 13493 | O | LYS | G | 571 | 97.029 | 84.368 | 10.777 | 1.00 | 88.42 | 8 |
| ATOM | 13494 | N | ARG | G | 572 | 97.131 | 85.879 | 9.110 | 1.00 | 94.32 | 7 |
| ATOM | 13495 | CA | ARG | G | 572 | 96.374 | 86.893 | 9.825 | 1.00 | 93.38 | 6 |
| ATOM | 13496 | CB | ARG | G | 572 | 96.379 | 88.210 | 9.061 | 1.00 | 83.79 | 6 |
| ATOM | 13497 | CG | ARG | G | 572 | 95.653 | 88.124 | 7.723 | 1.00 | 84.66 | 6 |
| ATOM | 13498 | CD | ARG | G | 572 | 95.120 | 89.481 | 7.279 | 1.00 | 86.66 | 6 |
| ATOM | 13499 | NE | ARG | G | 572 | 93.890 | 89.858 | 7.979 | 1.00 | 88.48 | 7 |
| ATOM | 13500 | CZ | ARG | G | 572 | 93.401 | 91.100 | 8.048 | 1.00 | 89.00 | 6 |
| ATOM | 13501 | NH1 | ARG | G | 572 | 94.034 | 92.114 | 7.463 | 1.00 | 89.06 | 7 |
| ATOM | 13502 | NH2 | ARG | G | 572 | 92.271 | 91.332 | 8.707 | 1.00 | 89.00 | 7 |
| ATOM | 13503 | C | ARG | G | 572 | 94.949 | 86.360 | 9.853 | 1.00 | 93.21 | 6 |
| ATOM | 13504 | O | ARG | G | 572 | 93.986 | 87.129 | 9.879 | 1.00 | 93.59 | 8 |
| ATOM | 13505 | N | MET | G | 573 | 94.826 | 85.036 | 9.823 | 1.00 | 126.59 | 7 |
| ATOM | 13506 | CA | MET | G | 573 | 93.528 | 84.381 | 9.825 | 1.00 | 125.83 | 6 |
| ATOM | 13507 | CB | MET | G | 573 | 93.123 | 84.034 | 8.391 | 1.00 | 190.37 | 6 |
| ATOM | 13508 | CG | MET | G | 573 | 93.054 | 85.242 | 7.469 | 1.00 | 192.13 | 6 |
| ATOM | 13509 | SD | MET | G | 573 | 92.546 | 84.815 | 5.797 | 1.00 | 193.34 | 16 |
| ATOM | 13510 | CE | MET | G | 573 | 94.118 | 84.474 | 5.040 | 1.00 | 193.04 | 6 |
| ATOM | 13511 | C | MET | G | 573 | 93.488 | 83.128 | 10.695 | 1.00 | 124.08 | 6 |
| ATOM | 13512 | O | MET | G | 573 | 92.455 | 82.818 | 11.276 | 1.00 | 123.42 | 8 |
| ATOM | 13513 | N | LEU | G | 574 | 94.599 | 82.402 | 10.787 | 1.00 | 97.74 | 7 |
| ATOM | 13514 | CA | LEU | G | 574 | 94.617 | 81.199 | 11.616 | 1.00 | 95.67 | 6 |
| ATOM | 13515 | CB | LEU | G | 574 | 96.014 | 80.610 | 11.747 | 1.00 | 46.39 | 6 |
| ATOM | 13516 | CG | LEU | G | 574 | 95.974 | 79.591 | 12.883 | 1.00 | 44.93 | 6 |
| ATOM | 13517 | CD1 | LEU | G | 574 | 94.919 | 78.560 | 12.538 | 1.00 | 44.33 | 6 |
| ATOM | 13518 | CD2 | LEU | G | 574 | 97.321 | 78.931 | 13.096 | 1.00 | 45.55 | 6 |
| ATOM | 13519 | C | LEU | G | 574 | 94.166 | 81.567 | 13.002 | 1.00 | 94.64 | 6 |
| ATOM | 13520 | O | LEU | G | 574 | 93.137 | 81.104 | 13.480 | 1.00 | 94.77 | 8 |
| ATOM | 13521 | N | GLN | G | 575 | 94.984 | 82.382 | 13.654 | 1.00 | 113.35 | 7 |
| ATOM | 13522 | CA | GLN | G | 575 | 94.683 | 82.846 | 14.991 | 1.00 | 112.36 | 6 |
| ATOM | 13523 | CB | GLN | G | 575 | 95.720 | 83.872 | 15.441 | 1.00 | 73.05 | 6 |
| ATOM | 13524 | CG | GLN | G | 575 | 95.247 | 84.677 | 16.625 | 1.00 | 72.56 | 6 |
| ATOM | 13525 | CD | GLN | G | 575 | 96.168 | 85.805 | 16.985 | 1.00 | 72.65 | 6 |
| ATOM | 13526 | OE1 | GLN | G | 575 | 97.267 | 85.581 | 17.485 | 1.00 | 74.21 | 8 |
| ATOM | 13527 | NE2 | GLN | G | 575 | 95.728 | 87.034 | 16.729 | 1.00 | 71.99 | 7 |
| ATOM | 13528 | C | GLN | G | 575 | 93.304 | 83.495 | 14.987 | 1.00 | 111.62 | 6 |
| ATOM | 13529 | O | GLN | G | 575 | 92.567 | 83.415 | 15.968 | 1.00 | 111.83 | 8 |
| ATOM | 13530 | N | GLU | G | 576 | 92.969 | 84.138 | 13.871 | 1.00 | 72.08 | 7 |
| ATOM | 13531 | CA | GLU | G | 576 | 91.690 | 84.813 | 13.715 | 1.00 | 70.28 | 6 |
| ATOM | 13532 | CB | GLU | G | 576 | 91.587 | 85.392 | 12.309 | 1.00 | 94.12 | 6 |
| ATOM | 13533 | CG | GLU | G | 576 | 90.913 | 86.732 | 12.248 | 1.00 | 94.40 | 6 |
| ATOM | 13534 | CD | GLU | G | 576 | 91.881 | 87.823 | 11.883 | 1.00 | 94.32 | 6 |
| ATOM | 13535 | OE1 | GLU | G | 576 | 92.966 | 87.871 | 12.500 | 1.00 | 94.33 | 8 |
| ATOM | 13536 | OE2 | GLU | G | 576 | 91.557 | 88.631 | 10.986 | 1.00 | 95.06 | 8 |
| ATOM | 13537 | C | GLU | G | 576 | 90.545 | 83.828 | 13.950 | 1.00 | 69.83 | 6 |
| ATOM | 13538 | O | GLU | G | 576 | 89.420 | 84.225 | 14.253 | 1.00 | 69.87 | 8 |
| ATOM | 13539 | N | ALA | G | 577 | 90.846 | 82.541 | 13.793 | 1.00 | 73.59 | 7 |
| ATOM | 13540 | CA | ALA | G | 577 | 89.867 | 81.476 | 13.992 | 1.00 | 72.77 | 6 |
| ATOM | 13541 | CB | ALA | G | 577 | 90.053 | 80.378 | 12.956 | 1.00 | 96.82 | 6 |
| ATOM | 13542 | C | ALA | G | 577 | 90.059 | 80.916 | 15.388 | 1.00 | 72.09 | 6 |
| ATOM | 13543 | O | ALA | G | 577 | 89.737 | 79.760 | 15.663 | 1.00 | 72.23 | 8 |
| ATOM | 13544 | N | VAL | G | 578 | 90.623 | 81.754 | 16.250 | 1.00 | 74.31 | 7 |
| ATOM | 13545 | CA | VAL | G | 578 | 90.854 | 81.437 | 17.654 | 1.00 | 74.39 | 6 |
| ATOM | 13546 | CB | VAL | G | 578 | 92.324 | 81.106 | 17.942 | 1.00 | 74.55 | 6 |
| ATOM | 13547 | CG1 | VAL | G | 578 | 92.443 | 80.437 | 19.311 | 1.00 | 73.52 | 6 |
| ATOM | 13548 | CG2 | VAL | G | 578 | 92.875 | 80.215 | 16.850 | 1.00 | 73.93 | 6 |
| ATOM | 13549 | C | VAL | G | 578 | 90.496 | 82.751 | 18.322 | 1.00 | 73.71 | 6 |
| ATOM | 13550 | O | VAL | G | 578 | 89.795 | 82.790 | 19.333 | 1.00 | 72.55 | 8 |
| ATOM | 13551 | N | ASP | G | 579 | 90.985 | 83.828 | 17.714 | 1.00 | 50.02 | 7 |
| ATOM | 13552 | CA | ASP | G | 579 | 90.714 | 85.186 | 18.161 | 1.00 | 51.25 | 6 |
| ATOM | 13553 | CB | ASP | G | 579 | 91.654 | 86.179 | 17.462 | 1.00 | 90.20 | 6 |
| ATOM | 13554 | CG | ASP | G | 579 | 93.040 | 86.228 | 18.088 | 1.00 | 90.68 | 6 |
| ATOM | 13555 | OD1 | ASP | G | 579 | 93.504 | 85.186 | 18.597 | 1.00 | 91.76 | 8 |
| ATOM | 13556 | OD2 | ASP | G | 579 | 93.670 | 87.311 | 18.051 | 1.00 | 90.12 | 8 |
| ATOM | 13557 | C | ASP | G | 579 | 89.274 | 85.455 | 17.737 | 1.00 | 51.80 | 6 |
| ATOM | 13558 | O | ASP | G | 579 | 88.894 | 86.592 | 17.413 | 1.00 | 51.23 | 8 |
| ATOM | 13559 | N | ALA | G | 580 | 88.500 | 84.370 | 17.720 | 1.00 | 91.07 | 7 |
| ATOM | 13560 | CA | ALA | G | 580 | 87.089 | 84.362 | 17.351 | 1.00 | 92.32 | 6 |
| ATOM | 13561 | CB | ALA | G | 580 | 86.935 | 84.093 | 15.850 | 1.00 | 107.39 | 6 |
| ATOM | 13562 | C | ALA | G | 580 | 86.424 | 83.247 | 18.161 | 1.00 | 93.13 | 6 |
| ATOM | 13563 | O | ALA | G | 580 | 85.423 | 83.470 | 18.846 | 1.00 | 93.04 | 8 |
| ATOM | 13564 | N | VAL | G | 581 | 86.991 | 82.045 | 18.074 | 1.00 | 139.52 | 7 |
| ATOM | 13565 | CA | VAL | G | 581 | 86.478 | 80.904 | 18.820 | 1.00 | 139.99 | 6 |
| ATOM | 13566 | CB | VAL | G | 581 | 87.451 | 79.722 | 18.766 | 1.00 | 30.99 | 6 |
| ATOM | 13567 | CG1 | VAL | G | 581 | 87.210 | 78.783 | 19.913 | 1.00 | 30.67 | 6 |
| ATOM | 13568 | CG2 | VAL | G | 581 | 87.266 | 78.980 | 17.485 | 1.00 | 30.60 | 6 |
| ATOM | 13569 | C | VAL | G | 581 | 86.326 | 81.339 | 20.261 | 1.00 | 141.61 | 6 |
| ATOM | 13570 | O | VAL | G | 581 | 85.299 | 81.088 | 20.891 | 1.00 | 142.75 | 8 |
| ATOM | 13571 | N | ILE | G | 582 | 87.362 | 81.992 | 20.778 | 1.00 | 51.60 | 7 |
| ATOM | 13572 | CA | ILE | G | 582 | 87.338 | 82.489 | 22.145 | 1.00 | 51.68 | 6 |
| ATOM | 13573 | CB | ILE | G | 582 | 88.734 | 82.902 | 22.568 | 1.00 | 65.92 | 6 |
| ATOM | 13574 | CG2 | ILE | G | 582 | 88.852 | 82.838 | 24.071 | 1.00 | 66.06 | 6 |
| ATOM | 13575 | CG1 | ILE | G | 582 | 89.744 | 81.937 | 21.949 | 1.00 | 67.19 | 6 |
| ATOM | 13576 | CD1 | ILE | G | 582 | 91.169 | 82.363 | 22.122 | 1.00 | 69.30 | 6 |
| ATOM | 13577 | C | ILE | G | 582 | 86.383 | 83.686 | 22.181 | 1.00 | 52.28 | 6 |
| ATOM | 13578 | O | ILE | G | 582 | 85.562 | 83.837 | 21.273 | 1.00 | 51.26 | 8 |
| ATOM | 13579 | N | ASP | G | 583 | 86.479 | 84.523 | 23.216 | 1.00 | 131.06 | 7 |
| ATOM | 13580 | CA | ASP | G | 583 | 85.603 | 85.691 | 23.346 | 1.00 | 132.06 | 6 |
| ATOM | 13581 | CB | ASP | G | 583 | 86.151 | 86.678 | 24.402 | 1.00 | 83.72 | 6 |
| ATOM | 13582 | CG | ASP | G | 583 | 86.781 | 85.974 | 25.620 | 1.00 | 83.81 | 6 |
| ATOM | 13583 | OD1 | ASP | G | 583 | 86.234 | 84.953 | 26.086 | 1.00 | 82.85 | 8 |
| ATOM | 13584 | OD2 | ASP | G | 583 | 87.826 | 86.450 | 26.126 | 1.00 | 83.67 | 8 |
| ATOM | 13585 | C | ASP | G | 583 | 85.466 | 86.397 | 21.990 | 1.00 | 133.07 | 6 |
| ATOM | 13586 | O | ASP | G | 583 | 86.054 | 87.457 | 21.771 | 1.00 | 133.98 | 8 |
| ATOM | 13587 | N | ASN | G | 584 | 84.684 | 85.793 | 21.091 | 1.00 | 37.65 | 7 |
| ATOM | 13588 | CA | ASN | G | 584 | 84.455 | 86.310 | 19.743 | 1.00 | 37.79 | 6 |
| ATOM | 13589 | CB | ASN | G | 584 | 83.503 | 85.395 | 18.987 | 1.00 | 75.68 | 6 |
| ATOM | 13590 | CG | ASN | G | 584 | 83.340 | 85.801 | 17.541 | 1.00 | 76.20 | 6 |
| ATOM | 13591 | OD1 | ASN | G | 584 | 83.192 | 86.982 | 17.229 | 1.00 | 75.74 | 8 |
| ATOM | 13592 | ND2 | ASN | G | 584 | 83.350 | 84.819 | 16.646 | 1.00 | 76.61 | 7 |
| ATOM | 13593 | C | ASN | G | 584 | 83.883 | 87.720 | 19.741 | 1.00 | 38.41 | 6 |
| ATOM | 13594 | O | ASN | G | 584 | 82.706 | 87.920 | 19.452 | 1.00 | 39.07 | 8 |
| ATOM | 13595 | N | GLY | G | 585 | 84.724 | 88.696 | 20.058 | 1.00 | 115.74 | 7 |
| ATOM | 13596 | CA | GLY | G | 585 | 84.278 | 90.074 | 20.082 | 1.00 | 118.24 | 6 |
| ATOM | 13597 | C | GLY | G | 585 | 85.223 | 90.972 | 19.314 | 1.00 | 119.97 | 6 |
| ATOM | 13598 | O | GLY | G | 585 | 85.832 | 91.871 | 19.890 | 1.00 | 120.15 | 8 |
| ATOM | 13599 | N | ARG | G | 586 | 85.356 | 90.714 | 18.015 | 1.00 | 118.75 | 7 |
| ATOM | 13600 | CA | ARG | G | 586 | 86.222 | 91.505 | 17.140 | 1.00 | 120.54 | 6 |
| ATOM | 13601 | CB | ARG | G | 586 | 87.639 | 90.906 | 17.073 | 1.00 | 106.36 | 6 |
| ATOM | 13602 | CG | ARG | G | 586 | 88.602 | 91.620 | 16.099 | 1.00 | 107.18 | 6 |
| ATOM | 13603 | CD | ARG | G | 586 | 89.992 | 90.977 | 16.115 | 1.00 | 107.40 | 6 |
| ATOM | 13604 | NE | ARG | G | 586 | 90.897 | 91.561 | 15.126 | 1.00 | 107.01 | 7 |
| ATOM | 13605 | CZ | ARG | G | 586 | 92.177 | 91.220 | 14.984 | 1.00 | 107.15 | 6 |
| ATOM | 13606 | NH1 | ARG | G | 586 | 92.715 | 90.294 | 15.771 | 1.00 | 106.98 | 7 |
| ATOM | 13607 | NH2 | ARG | G | 586 | 92.924 | 91.800 | 14.051 | 1.00 | 106.67 | 7 |
| ATOM | 13608 | C | ARG | G | 586 | 85.612 | 91.529 | 15.747 | 1.00 | 120.97 | 6 |
| ATOM | 13609 | O | ARG | G | 586 | 84.997 | 90.555 | 15.325 | 1.00 | 121.83 | 8 |
| ATOM | 13610 | N | ARG | G | 587 | 85.788 | 92.648 | 15.050 | 1.00 | 104.27 | 7 |
| ATOM | 13611 | CA | ARG | G | 587 | 85.271 | 92.843 | 13.701 | 1.00 | 104.71 | 6 |
| ATOM | 13612 | CB | ARG | G | 587 | 86.153 | 92.109 | 12.676 | 1.00 | 114.96 | 6 |
| ATOM | 13613 | CG | ARG | G | 587 | 86.009 | 90.585 | 12.626 | 1.00 | 115.33 | 6 |
| ATOM | 13614 | CD | ARG | G | 587 | 87.070 | 89.850 | 13.451 | 1.00 | 115.65 | 6 |
| ATOM | 13615 | NE | ARG | G | 587 | 86.981 | 88.399 | 13.267 | 1.00 | 116.31 | 7 |
| ATOM | 13616 | CZ | ARG | G | 587 | 87.703 | 87.500 | 13.932 | 1.00 | 116.40 | 6 |
| ATOM | 13617 | NH1 | ARG | G | 587 | 88.587 | 87.884 | 14.845 | 1.00 | 116.24 | 7 |
| ATOM | 13618 | NH2 | ARG | G | 587 | 87.538 | 86.209 | 13.684 | 1.00 | 116.24 | 7 |
| ATOM | 13619 | C | ARG | G | 587 | 83.807 | 92.439 | 13.508 | 1.00 | 104.86 | 6 |
| ATOM | 13620 | O | ARG | G | 587 | 83.000 | 93.239 | 13.032 | 1.00 | 105.52 | 8 |
| ATOM | 13621 | N | GLY | G | 588 | 83.476 | 91.204 | 13.878 | 1.00 | 147.52 | 7 |
| ATOM | 13622 | CA | GLY | G | 588 | 82.123 | 90.693 | 13.742 | 1.00 | 146.07 | 6 |
| ATOM | 13623 | C | GLY | G | 588 | 82.143 | 89.457 | 12.867 | 1.00 | 144.98 | 6 |
| ATOM | 13624 | O | GLY | G | 588 | 82.038 | 89.563 | 11.647 | 1.00 | 145.06 | 8 |
| ATOM | 13625 | N | SER | G | 589 | 82.271 | 88.284 | 13.480 | 1.00 | 77.21 | 7 |
| ATOM | 13626 | CA | SER | G | 589 | 82.338 | 87.037 | 12.718 | 1.00 | 76.00 | 6 |
| ATOM | 13627 | CB | SER | G | 589 | 83.639 | 86.302 | 13.050 | 1.00 | 56.13 | 6 |
| ATOM | 13628 | OG | SER | G | 589 | 83.756 | 86.073 | 14.445 | 1.00 | 55.41 | 8 |
| ATOM | 13629 | C | SER | G | 589 | 81.160 | 86.070 | 12.868 | 1.00 | 75.43 | 6 |
| ATOM | 13630 | O | SER | G | 589 | 81.151 | 85.207 | 13.751 | 1.00 | 75.27 | 8 |
| ATOM | 13631 | N | PRO | G | 590 | 80.146 | 86.209 | 11.996 | 1.00 | 65.35 | 7 |
| ATOM | 13632 | CD | PRO | G | 590 | 79.925 | 87.427 | 11.189 | 1.00 | 140.99 | 6 |
| ATOM | 13633 | CA | PRO | G | 590 | 78.944 | 85.368 | 11.990 | 1.00 | 65.07 | 6 |
| ATOM | 13634 | CB | PRO | G | 590 | 77.860 | 86.350 | 11.568 | 1.00 | 140.82 | 6 |
| ATOM | 13635 | CG | PRO | G | 590 | 78.572 | 87.164 | 10.539 | 1.00 | 140.67 | 6 |
| ATOM | 13636 | C | PRO | G | 590 | 79.054 | 84.193 | 11.015 | 1.00 | 64.88 | 6 |
| ATOM | 13637 | O | PRO | G | 590 | 79.968 | 83.371 | 11.099 | 1.00 | 64.41 | 8 |
| ATOM | 13638 | N | VAL | G | 591 | 78.096 | 84.132 | 10.095 | 1.00 | 120.37 | 7 |
| ATOM | 13639 | CA | VAL | G | 591 | 78.040 | 83.099 | 9.071 | 1.00 | 120.69 | 6 |
| ATOM | 13640 | CB | VAL | G | 591 | 77.004 | 81.994 | 9.424 | 1.00 | 83.72 | 6 |
| ATOM | 13641 | CG1 | VAL | G | 591 | 75.593 | 82.547 | 9.375 | 1.00 | 83.05 | 6 |
| ATOM | 13642 | CG2 | VAL | G | 591 | 77.144 | 80.835 | 8.474 | 1.00 | 83.74 | 6 |
| ATOM | 13643 | C | VAL | G | 591 | 77.632 | 83.792 | 7.775 | 1.00 | 120.85 | 6 |
| ATOM | 13644 | O | VAL | G | 591 | 77.204 | 84.948 | 7.796 | 1.00 | 120.97 | 8 |
| ATOM | 13645 | N | THR | G | 592 | 77.770 | 83.088 | 6.655 | 1.00 | 117.86 | 7 |
| ATOM | 13646 | CA | THR | G | 592 | 77.432 | 83.631 | 5.339 | 1.00 | 117.55 | 6 |
| ATOM | 13647 | CB | THR | G | 592 | 75.935 | 83.397 | 4.992 | 1.00 | 124.78 | 6 |
| ATOM | 13648 | OG1 | THR | G | 592 | 75.108 | 83.952 | 6.023 | 1.00 | 125.06 | 8 |
| ATOM | 13649 | CG2 | THR | G | 592 | 75.641 | 81.905 | 4.844 | 1.00 | 123.79 | 6 |
| ATOM | 13650 | C | THR | G | 592 | 77.740 | 85.128 | 5.222 | 1.00 | 117.06 | 6 |
| ATOM | 13651 | O | THR | G | 592 | 76.832 | 85.958 | 5.169 | 1.00 | 117.05 | 8 |
| ATOM | 13652 | N | ASN | G | 593 | 79.030 | 85.459 | 5.185 | 1.00 | 166.11 | 7 |
| ATOM | 13653 | CA | ASN | G | 593 | 79.479 | 86.845 | 5.066 | 1.00 | 166.57 | 6 |
| ATOM | 13654 | CB | ASN | G | 593 | 80.903 | 86.986 | 5.615 | 1.00 | 110.57 | 6 |
| ATOM | 13655 | CG | ASN | G | 593 | 80.951 | 86.946 | 7.131 | 1.00 | 110.95 | 6 |
| ATOM | 13656 | OD1 | ASN | G | 593 | 80.512 | 87.881 | 7.806 | 1.00 | 110.65 | 8 |
| ATOM | 13657 | ND2 | ASN | G | 593 | 81.484 | 85.858 | 7.676 | 1.00 | 111.38 | 7 |
| ATOM | 13658 | C | ASN | G | 593 | 79.461 | 87.285 | 3.603 | 1.00 | 166.40 | 6 |
| ATOM | 13659 | O | ASN | G | 593 | 79.396 | 86.454 | 2.698 | 1.00 | 167.00 | 8 |
| ATOM | 13660 | N | PRO | G | 594 | 79.511 | 88.605 | 3.352 | 0.00 | 83.88 | 7 |
| ATOM | 13661 | CD | PRO | G | 594 | 79.851 | 89.122 | 2.011 | 0.00 | 83.46 | 6 |
| ATOM | 13662 | CA | PRO | G | 594 | 79.604 | 89.677 | 4.348 | 0.00 | 83.09 | 6 |
| ATOM | 13663 | CB | PRO | G | 594 | 80.494 | 90.695 | 3.658 | 0.00 | 83.02 | 6 |
| ATOM | 13664 | CG | PRO | G | 594 | 79.994 | 90.620 | 2.253 | 0.00 | 83.14 | 6 |
| ATOM | 13665 | C | PRO | G | 594 | 78.246 | 90.275 | 4.731 | 0.00 | 82.44 | 6 |
| ATOM | 13666 | O | PRO | G | 594 | 78.135 | 90.983 | 5.732 | 0.00 | 82.37 | 8 |
| ATOM | 13667 | N | GLY | G | 595 | 77.226 | 89.989 | 3.926 | 0.00 | 81.94 | 7 |
| ATOM | 13668 | CA | GLY | G | 595 | 75.892 | 90.511 | 4.178 | 0.00 | 81.22 | 6 |
| ATOM | 13669 | C | GLY | G | 595 | 75.498 | 90.623 | 5.639 | 0.00 | 80.87 | 6 |
| ATOM | 13670 | O | GLY | G | 595 | 75.834 | 89.757 | 6.449 | 0.00 | 80.75 | 8 |
| ATOM | 13671 | N | SER | G | 596 | 74.785 | 91.696 | 5.974 | 0.00 | 80.35 | 7 |
| ATOM | 13672 | CA | SER | G | 596 | 74.330 | 91.933 | 7.342 | 0.00 | 79.99 | 6 |
| ATOM | 13673 | CB | SER | G | 596 | 73.318 | 93.080 | 7.370 | 0.00 | 80.14 | 6 |
| ATOM | 13674 | OG | SER | G | 596 | 72.193 | 92.787 | 6.560 | 0.00 | 80.12 | 8 |
| ATOM | 13675 | C | SER | G | 596 | 73.690 | 90.666 | 7.897 | 0.00 | 79.81 | 6 |
| ATOM | 13676 | O | SER | G | 596 | 73.007 | 89.944 | 7.170 | 0.00 | 79.83 | 8 |
| ATOM | 13677 | N | GLU | G | 597 | 73.904 | 90.405 | 9.185 | 1.00 | 122.38 | 7 |
| ATOM | 13678 | CA | GLU | G | 597 | 73.364 | 89.205 | 9.815 | 1.00 | 121.70 | 6 |
| ATOM | 13679 | CB | GLU | G | 597 | 73.821 | 87.986 | 9.017 | 1.00 | 92.65 | 6 |
| ATOM | 13680 | CG | GLU | G | 597 | 73.235 | 86.671 | 9.445 | 1.00 | 92.04 | 6 |
| ATOM | 13681 | CD | GLU | G | 597 | 73.634 | 85.566 | 8.503 | 1.00 | 91.30 | 6 |
| ATOM | 13682 | OE1 | GLU | G | 597 | 74.853 | 85.364 | 8.315 | 1.00 | 90.02 | 8 |
| ATOM | 13683 | OE2 | GLU | G | 597 | 72.731 | 84.908 | 7.946 | 1.00 | 90.79 | 8 |
| ATOM | 13684 | C | GLU | G | 597 | 73.828 | 89.086 | 11.269 | 1.00 | 121.10 | 6 |
| ATOM | 13685 | O | GLU | G | 597 | 74.678 | 89.856 | 11.721 | 1.00 | 121.55 | 8 |
| ATOM | 13686 | N | ARG | G | 598 | 73.269 | 88.120 | 11.995 | 1.00 | 117.35 | 7 |
| ATOM | 13687 | CA | ARG | G | 598 | 73.623 | 87.897 | 13.399 | 1.00 | 116.12 | 6 |
| ATOM | 13688 | CB | ARG | G | 598 | 72.471 | 87.210 | 14.133 | 1.00 | 155.91 | 6 |
| ATOM | 13689 | CG | ARG | G | 598 | 71.128 | 87.919 | 14.033 | 1.00 | 157.25 | 6 |
| ATOM | 13690 | CD | ARG | G | 598 | 70.044 | 87.035 | 14.628 | 1.00 | 159.10 | 6 |
| ATOM | 13691 | NE | ARG | G | 598 | 68.698 | 87.541 | 14.386 | 1.00 | 160.81 | 7 |
| ATOM | 13692 | CZ | ARG | G | 598 | 67.591 | 86.846 | 14.629 | 1.00 | 161.53 | 6 |
| ATOM | 13693 | NH1 | ARG | G | 598 | 67.672 | 85.613 | 15.119 | 1.00 | 161.89 | 7 |
| ATOM | 13694 | NH2 | ARG | G | 598 | 66.400 | 87.379 | 14.381 | 1.00 | 161.59 | 7 |
| ATOM | 13695 | C | ARG | G | 598 | 74.878 | 87.028 | 13.517 | 1.00 | 114.07 | 6 |
| ATOM | 13696 | O | ARG | G | 598 | 75.023 | 86.037 | 12.800 | 1.00 | 114.47 | 8 |
| ATOM | 13697 | N | PRO | G | 599 | 75.789 | 87.381 | 14.443 | 1.00 | 59.23 | 7 |
| ATOM | 13698 | CD | PRO | G | 599 | 75.586 | 88.420 | 15.466 | 1.00 | 48.11 | 6 |
| ATOM | 13699 | CA | PRO | G | 599 | 77.044 | 86.660 | 14.682 | 1.00 | 58.47 | 6 |
| ATOM | 13700 | CB | PRO | G | 599 | 77.760 | 87.546 | 15.687 | 1.00 | 47.91 | 6 |
| ATOM | 13701 | CG | PRO | G | 599 | 76.625 | 88.060 | 16.509 | 1.00 | 47.94 | 6 |
| ATOM | 13702 | C | PRO | G | 599 | 76.793 | 85.259 | 15.211 | 1.00 | 58.22 | 6 |
| ATOM | 13703 | O | PRO | G | 599 | 75.707 | 84.718 | 15.028 | 1.00 | 59.47 | 8 |
| ATOM | 13704 | N | LEU | G | 600 | 77.784 | 84.668 | 15.869 | 1.00 | 48.10 | 7 |
| ATOM | 13705 | CA | LEU | G | 600 | 77.619 | 83.314 | 16.378 | 1.00 | 47.99 | 6 |
| ATOM | 13706 | CB | LEU | G | 600 | 78.587 | 82.386 | 15.666 | 1.00 | 54.05 | 6 |
| ATOM | 13707 | CG | LEU | G | 600 | 78.820 | 82.837 | 14.226 | 1.00 | 54.55 | 6 |
| ATOM | 13708 | CD1 | LEU | G | 600 | 79.875 | 81.967 | 13.581 | 1.00 | 55.60 | 6 |
| ATOM | 13709 | CD2 | LEU | G | 600 | 77.524 | 82.773 | 13.452 | 1.00 | 53.38 | 6 |
| ATOM | 13710 | C | LEU | G | 600 | 77.854 | 83.237 | 17.875 | 1.00 | 48.55 | 6 |
| ATOM | 13711 | O | LEU | G | 600 | 78.329 | 84.189 | 18.490 | 1.00 | 48.30 | 8 |
| ATOM | 13712 | N | ARG | G | 601 | 77.509 | 82.100 | 18.466 | 1.00 | 74.02 | 7 |
| ATOM | 13713 | CA | ARG | G | 601 | 77.704 | 81.923 | 19.893 | 1.00 | 74.67 | 6 |
| ATOM | 13714 | CB | ARG | G | 601 | 76.612 | 81.027 | 20.488 | 1.00 | 83.57 | 6 |
| ATOM | 13715 | CG | ARG | G | 601 | 75.179 | 81.541 | 20.320 | 1.00 | 85.04 | 6 |
| ATOM | 13716 | CD | ARG | G | 601 | 75.046 | 83.009 | 20.711 | 1.00 | 85.98 | 6 |
| ATOM | 13717 | NE | ARG | G | 601 | 73.657 | 83.454 | 20.825 | 1.00 | 87.40 | 7 |
| ATOM | 13718 | CZ | ARG | G | 601 | 73.293 | 84.735 | 20.853 | 1.00 | 88.26 | 6 |
| ATOM | 13719 | NH1 | ARG | G | 601 | 74.219 | 85.686 | 20.767 | 1.00 | 88.90 | 7 |
| ATOM | 13720 | NH2 | ARG | G | 601 | 72.013 | 85.072 | 20.980 | 1.00 | 88.70 | 7 |
| ATOM | 13721 | C | ARG | G | 601 | 79.066 | 81.284 | 20.083 | 1.00 | 74.96 | 6 |
| ATOM | 13722 | O | ARG | G | 601 | 79.223 | 80.076 | 19.954 | 1.00 | 75.24 | 8 |
| ATOM | 13723 | N | SER | G | 602 | 80.061 | 82.103 | 20.377 | 1.00 | 52.21 | 7 |
| ATOM | 13724 | CA | SER | G | 602 | 81.406 | 81.588 | 20.567 | 1.00 | 53.74 | 6 |
| ATOM | 13725 | CB | SER | G | 602 | 82.416 | 82.736 | 20.484 | 1.00 | 113.06 | 6 |
| ATOM | 13726 | OG | SER | G | 602 | 81.981 | 83.859 | 21.230 | 1.00 | 113.37 | 8 |
| ATOM | 13727 | C | SER | G | 602 | 81.556 | 80.842 | 21.890 | 1.00 | 54.78 | 6 |
| ATOM | 13728 | O | SER | G | 602 | 80.597 | 80.732 | 22.651 | 1.00 | 55.37 | 8 |
| ATOM | 13729 | N | LEU | G | 603 | 82.762 | 80.336 | 22.152 | 1.00 | 68.31 | 7 |
| ATOM | 13730 | CA | LEU | G | 603 | 83.054 | 79.604 | 23.380 | 1.00 | 69.95 | 6 |
| ATOM | 13731 | CB | LEU | G | 603 | 84.533 | 79.211 | 23.436 | 1.00 | 66.38 | 6 |
| ATOM | 13732 | CG | LEU | G | 603 | 84.814 | 77.704 | 23.484 | 1.00 | 67.10 | 6 |
| ATOM | 13733 | CD1 | LEU | G | 603 | 84.313 | 77.098 | 22.211 | 1.00 | 66.06 | 6 |
| ATOM | 13734 | CD2 | LEU | G | 603 | 86.297 | 77.407 | 23.628 | 1.00 | 67.79 | 6 |
| ATOM | 13735 | C | LEU | G | 603 | 82.709 | 80.397 | 24.627 | 1.00 | 71.38 | 6 |
| ATOM | 13736 | O | LEU | G | 603 | 82.408 | 79.820 | 25.658 | 1.00 | 72.13 | 8 |
| ATOM | 13737 | N | THR | G | 604 | 82.750 | 81.719 | 24.534 | 1.00 | 131.25 | 7 |
| ATOM | 13738 | CA | THR | G | 604 | 82.440 | 82.563 | 25.682 | 1.00 | 132.27 | 6 |
| ATOM | 13739 | CB | THR | G | 604 | 83.017 | 83.968 | 25.495 | 1.00 | 142.86 | 6 |
| ATOM | 13740 | OG1 | THR | G | 604 | 84.421 | 83.864 | 25.245 | 1.00 | 143.74 | 8 |
| ATOM | 13741 | CG2 | THR | G | 604 | 82.790 | 84.811 | 26.738 | 1.00 | 142.56 | 6 |
| ATOM | 13742 | C | THR | G | 604 | 80.941 | 82.673 | 25.949 | 1.00 | 132.56 | 6 |
| ATOM | 13743 | O | THR | G | 604 | 80.528 | 83.008 | 27.057 | 1.00 | 133.04 | 8 |
| ATOM | 13744 | N | ASP | G | 605 | 80.125 | 82.403 | 24.936 | 1.00 | 50.88 | 7 |
| ATOM | 13745 | CA | ASP | G | 605 | 78.691 | 82.466 | 25.120 | 1.00 | 51.18 | 6 |
| ATOM | 13746 | CB | ASP | G | 605 | 77.968 | 82.432 | 23.775 | 1.00 | 208.87 | 6 |
| ATOM | 13747 | CG | ASP | G | 605 | 76.464 | 82.440 | 23.930 | 1.00 | 208.87 | 6 |
| ATOM | 13748 | OD1 | ASP | G | 605 | 75.879 | 81.355 | 24.142 | 1.00 | 208.87 | 8 |
| ATOM | 13749 | OD2 | ASP | G | 605 | 75.864 | 83.534 | 23.855 | 1.00 | 208.87 | 8 |
| ATOM | 13750 | C | ASP | G | 605 | 78.295 | 81.268 | 25.976 | 1.00 | 49.56 | 6 |
| ATOM | 13751 | O | ASP | G | 605 | 77.341 | 81.324 | 26.742 | 1.00 | 49.51 | 8 |
| ATOM | 13752 | N | ILE | G | 606 | 79.026 | 80.173 | 25.851 | 1.00 | 41.61 | 7 |
| ATOM | 13753 | CA | ILE | G | 606 | 78.713 | 79.016 | 26.670 | 1.00 | 41.65 | 6 |
| ATOM | 13754 | CB | ILE | G | 606 | 79.506 | 77.753 | 26.245 | 1.00 | 44.09 | 6 |
| ATOM | 13755 | CG2 | ILE | G | 606 | 79.121 | 76.572 | 27.120 | 1.00 | 42.98 | 6 |
| ATOM | 13756 | CG1 | ILE | G | 606 | 79.228 | 77.416 | 24.783 | 1.00 | 43.64 | 6 |
| ATOM | 13757 | CD1 | ILE | G | 606 | 80.000 | 78.255 | 23.791 | 1.00 | 43.28 | 6 |
| ATOM | 13758 | C | ILE | G | 606 | 79.189 | 79.409 | 28.050 | 1.00 | 42.41 | 6 |
| ATOM | 13759 | O | ILE | G | 606 | 78.831 | 78.796 | 29.041 | 1.00 | 43.00 | 8 |
| ATOM | 13760 | N | LEU | G | 607 | 79.995 | 80.458 | 28.104 | 1.00 | 91.80 | 7 |
| ATOM | 13761 | CA | LEU | G | 607 | 80.565 | 80.904 | 29.359 | 1.00 | 93.30 | 6 |
| ATOM | 13762 | CB | LEU | G | 607 | 82.072 | 80.668 | 29.315 | 1.00 | 64.66 | 6 |
| ATOM | 13763 | CG | LEU | G | 607 | 82.801 | 80.121 | 30.528 | 1.00 | 65.41 | 6 |
| ATOM | 13764 | CD1 | LEU | G | 607 | 82.646 | 78.606 | 30.580 | 1.00 | 65.76 | 6 |
| ATOM | 13765 | CD2 | LEU | G | 607 | 84.261 | 80.495 | 30.417 | 1.00 | 64.60 | 6 |
| ATOM | 13766 | C | LEU | G | 607 | 80.312 | 82.382 | 29.619 | 1.00 | 94.48 | 6 |
| ATOM | 13767 | O | LEU | G | 607 | 81.271 | 83.128 | 29.801 | 1.00 | 96.06 | 8 |
| ATOM | 13768 | N | SER | G | 608 | 79.055 | 82.822 | 29.633 | 1.00 | 44.59 | 7 |
| ATOM | 13769 | CA | SER | G | 608 | 78.794 | 84.239 | 29.884 | 1.00 | 45.43 | 6 |
| ATOM | 13770 | CB | SER | G | 608 | 79.662 | 85.103 | 28.970 | 1.00 | 65.77 | 6 |
| ATOM | 13771 | OG | SER | G | 608 | 79.407 | 86.480 | 29.208 | 1.00 | 67.77 | 8 |
| ATOM | 13772 | C | SER | G | 608 | 77.367 | 84.772 | 29.793 | 1.00 | 45.67 | 6 |
| ATOM | 13773 | O | SER | G | 608 | 76.537 | 84.283 | 29.030 | 1.00 | 45.55 | 8 |
| ATOM | 13774 | N | GLY | G | 609 | 77.127 | 85.819 | 30.578 | 1.00 | 61.64 | 7 |
| ATOM | 13775 | CA | GLY | G | 609 | 75.846 | 86.501 | 30.631 | 1.00 | 63.15 | 6 |
| ATOM | 13776 | C | GLY | G | 609 | 74.603 | 85.718 | 30.285 | 1.00 | 64.26 | 6 |
| ATOM | 13777 | O | GLY | G | 609 | 74.284 | 84.720 | 30.932 | 1.00 | 65.19 | 8 |
| ATOM | 13778 | N | LYS | G | 610 | 73.897 | 86.206 | 29.267 | 1.00 | 208.87 | 7 |
| ATOM | 13779 | CA | LYS | G | 610 | 72.662 | 85.614 | 28.758 | 1.00 | 208.87 | 6 |
| ATOM | 13780 | CB | LYS | G | 610 | 72.487 | 86.017 | 27.290 | 1.00 | 208.87 | 6 |
| ATOM | 13781 | CG | LYS | G | 610 | 72.637 | 87.516 | 27.050 | 1.00 | 208.87 | 6 |
| ATOM | 13782 | CD | LYS | G | 610 | 72.744 | 87.847 | 25.569 | 1.00 | 208.87 | 6 |
| ATOM | 13783 | CE | LYS | G | 610 | 73.053 | 89.323 | 25.356 | 1.00 | 208.87 | 6 |
| ATOM | 13784 | NZ | LYS | G | 610 | 73.256 | 89.668 | 23.920 | 1.00 | 208.87 | 7 |
| ATOM | 13785 | C | LYS | G | 610 | 72.655 | 84.092 | 28.887 | 1.00 | 208.87 | 6 |
| ATOM | 13786 | O | LYS | G | 610 | 73.001 | 83.379 | 27.946 | 1.00 | 208.87 | 8 |
| ATOM | 13787 | N | GLN | G | 611 | 72.251 | 83.601 | 30.054 | 1.00 | 48.76 | 7 |
| ATOM | 13788 | CA | GLN | G | 611 | 72.221 | 82.168 | 30.310 | 1.00 | 46.20 | 6 |
| ATOM | 13789 | CB | GLN | G | 611 | 71.138 | 81.495 | 29.456 | 1.00 | 184.13 | 6 |
| ATOM | 13790 | CG | GLN | G | 611 | 69.726 | 81.960 | 29.775 | 1.00 | 186.33 | 6 |
| ATOM | 13791 | CD | GLN | G | 611 | 68.661 | 81.026 | 29.230 | 1.00 | 187.53 | 6 |
| ATOM | 13792 | OE1 | GLN | G | 611 | 68.603 | 79.850 | 29.596 | 1.00 | 188.49 | 8 |
| ATOM | 13793 | NE2 | GLN | G | 611 | 67.808 | 81.547 | 28.355 | 1.00 | 187.68 | 7 |
| ATOM | 13794 | C | GLN | G | 611 | 73.568 | 81.464 | 30.086 | 1.00 | 43.77 | 6 |
| ATOM | 13795 | O | GLN | G | 611 | 73.749 | 80.758 | 29.097 | 1.00 | 43.16 | 8 |
| ATOM | 13796 | N | GLY | G | 612 | 74.507 | 81.653 | 31.010 | 1.00 | 87.49 | 7 |
| ATOM | 13797 | CA | GLY | G | 612 | 75.807 | 81.009 | 30.896 | 1.00 | 84.48 | 6 |
| ATOM | 13798 | C | GLY | G | 612 | 76.013 | 80.040 | 32.046 | 1.00 | 82.41 | 6 |
| ATOM | 13799 | O | GLY | G | 612 | 75.470 | 80.264 | 33.127 | 1.00 | 82.87 | 8 |
| ATOM | 13800 | N | ARG | G | 613 | 76.792 | 78.978 | 31.828 | 1.00 | 92.68 | 7 |
| ATOM | 13801 | CA | ARG | G | 613 | 77.053 | 77.968 | 32.860 | 1.00 | 90.80 | 6 |
| ATOM | 13802 | CB | ARG | G | 613 | 78.222 | 77.059 | 32.470 | 1.00 | 152.76 | 6 |
| ATOM | 13803 | CG | ARG | G | 613 | 77.881 | 76.013 | 31.427 | 1.00 | 156.97 | 6 |
| ATOM | 13804 | CD | ARG | G | 613 | 76.751 | 75.093 | 31.874 | 1.00 | 161.19 | 6 |
| ATOM | 13805 | NE | ARG | G | 613 | 76.377 | 74.168 | 30.803 | 1.00 | 165.23 | 7 |
| ATOM | 13806 | CZ | ARG | G | 613 | 75.351 | 73.323 | 30.854 | 1.00 | 166.73 | 6 |
| ATOM | 13807 | NH1 | ARG | G | 613 | 74.577 | 73.271 | 31.931 | 1.00 | 166.77 | 7 |
| ATOM | 13808 | NH2 | ARG | G | 613 | 75.095 | 72.530 | 29.821 | 1.00 | 167.54 | 7 |
| ATOM | 13809 | C | ARG | G | 613 | 77.328 | 78.549 | 34.232 | 1.00 | 88.05 | 6 |
| ATOM | 13810 | O | ARG | G | 613 | 77.528 | 77.803 | 35.186 | 1.00 | 88.02 | 8 |
| ATOM | 13811 | N | PHE | G | 614 | 77.361 | 79.876 | 34.323 | 1.00 | 74.36 | 7 |
| ATOM | 13812 | CA | PHE | G | 614 | 77.571 | 80.556 | 35.592 | 1.00 | 70.51 | 6 |
| ATOM | 13813 | CB | PHE | G | 614 | 78.508 | 81.763 | 35.430 | 1.00 | 47.48 | 6 |
| ATOM | 13814 | CG | PHE | G | 614 | 79.975 | 81.402 | 35.405 | 1.00 | 47.07 | 6 |
| ATOM | 13815 | CD1 | PHE | G | 614 | 80.766 | 81.712 | 34.303 | 1.00 | 46.72 | 6 |
| ATOM | 13816 | CD2 | PHE | G | 614 | 80.562 | 80.737 | 36.478 | 1.00 | 47.03 | 6 |
| ATOM | 13817 | CE1 | PHE | G | 614 | 82.110 | 81.366 | 34.267 | 1.00 | 47.18 | 6 |
| ATOM | 13818 | CE2 | PHE | G | 614 | 81.909 | 80.385 | 36.451 | 1.00 | 47.23 | 6 |
| ATOM | 13819 | CZ | PHE | G | 614 | 82.680 | 80.699 | 35.346 | 1.00 | 47.41 | 6 |
| ATOM | 13820 | C | PHE | G | 614 | 76.215 | 81.019 | 36.097 | 1.00 | 67.81 | 6 |
| ATOM | 13821 | O | PHE | G | 614 | 75.697 | 80.505 | 37.084 | 1.00 | 68.35 | 8 |
| ATOM | 13822 | N | ARG | G | 615 | 75.626 | 81.974 | 35.396 | 1.00 | 43.81 | 7 |
| ATOM | 13823 | CA | ARG | G | 615 | 74.339 | 82.505 | 35.803 | 1.00 | 41.47 | 6 |
| ATOM | 13824 | CB | ARG | G | 615 | 73.980 | 83.706 | 34.942 | 1.00 | 73.46 | 6 |
| ATOM | 13825 | CG | ARG | G | 615 | 75.068 | 84.759 | 34.904 | 1.00 | 74.60 | 6 |
| ATOM | 13826 | CD | ARG | G | 615 | 74.486 | 86.089 | 34.520 | 1.00 | 73.90 | 6 |
| ATOM | 13827 | NE | ARG | G | 615 | 75.421 | 86.904 | 33.762 | 1.00 | 74.07 | 7 |
| ATOM | 13828 | CZ | ARG | G | 615 | 75.088 | 88.058 | 33.193 | 1.00 | 74.42 | 6 |
| ATOM | 13829 | NH1 | ARG | G | 615 | 73.843 | 88.515 | 33.315 | 1.00 | 74.48 | 7 |
| ATOM | 13830 | NH2 | ARG | G | 615 | 75.983 | 88.746 | 32.486 | 1.00 | 75.26 | 7 |
| ATOM | 13831 | C | ARG | G | 615 | 73.195 | 81.495 | 35.773 | 1.00 | 39.60 | 6 |
| ATOM | 13832 | O | ARG | G | 615 | 72.223 | 81.632 | 36.517 | 1.00 | 39.50 | 8 |
| ATOM | 13833 | N | GLN | G | 616 | 73.298 | 80.482 | 34.923 | 1.00 | 37.71 | 7 |
| ATOM | 13834 | CA | GLN | G | 616 | 72.242 | 79.476 | 34.838 | 1.00 | 34.81 | 6 |
| ATOM | 13835 | CB | GLN | G | 616 | 72.452 | 78.589 | 33.614 | 1.00 | 86.50 | 6 |
| ATOM | 13836 | CG | GLN | G | 616 | 71.372 | 77.554 | 33.432 | 1.00 | 91.00 | 6 |
| ATOM | 13837 | CD | GLN | G | 616 | 71.306 | 77.045 | 32.016 | 1.00 | 94.14 | 6 |
| ATOM | 13838 | OE1 | GLN | G | 616 | 71.019 | 77.801 | 31.082 | 1.00 | 95.38 | 8 |
| ATOM | 13839 | NE2 | GLN | G | 616 | 71.572 | 75.756 | 31.842 | 1.00 | 96.47 | 7 |
| ATOM | 13840 | C | GLN | G | 616 | 72.271 | 78.636 | 36.096 | 1.00 | 30.75 | 6 |
| ATOM | 13841 | O | GLN | G | 616 | 73.195 | 77.868 | 36.300 | 1.00 | 29.90 | 8 |
| ATOM | 13842 | N | ASN | G | 617 | 71.248 | 78.790 | 36.922 | 1.00 | 16.17 | 7 |
| ATOM | 13843 | CA | ASN | G | 617 | 71.115 | 78.101 | 38.200 | 1.00 | 13.87 | 6 |
| ATOM | 13844 | CB | ASN | G | 617 | 72.425 | 77.467 | 38.696 | 1.00 | 18.24 | 6 |
| ATOM | 13845 | CG | ASN | G | 617 | 72.580 | 76.019 | 38.283 | 1.00 | 16.86 | 6 |
| ATOM | 13846 | OD1 | ASN | G | 617 | 71.946 | 75.567 | 37.345 | 1.00 | 16.30 | 8 |
| ATOM | 13847 | ND2 | ASN | G | 617 | 73.448 | 75.291 | 38.973 | 1.00 | 17.10 | 7 |
| ATOM | 13848 | C | ASN | G | 617 | 70.769 | 79.242 | 39.121 | 1.00 | 13.87 | 6 |
| ATOM | 13849 | O | ASN | G | 617 | 69.851 | 79.149 | 39.922 | 1.00 | 13.87 | 8 |
| ATOM | 13850 | N | LEU | G | 618 | 71.517 | 80.328 | 39.011 | 1.00 | 16.18 | 7 |
| ATOM | 13851 | CA | LEU | G | 618 | 71.251 | 81.484 | 39.832 | 1.00 | 16.59 | 6 |
| ATOM | 13852 | CB | LEU | G | 618 | 72.238 | 82.575 | 39.477 | 1.00 | 16.61 | 6 |
| ATOM | 13853 | CG | LEU | G | 618 | 73.621 | 82.082 | 39.862 | 1.00 | 16.77 | 6 |
| ATOM | 13854 | CD1 | LEU | G | 618 | 74.719 | 82.906 | 39.228 | 1.00 | 14.48 | 6 |
| ATOM | 13855 | CD2 | LEU | G | 618 | 73.692 | 82.102 | 41.378 | 1.00 | 16.76 | 6 |
| ATOM | 13856 | C | LEU | G | 618 | 69.832 | 81.867 | 39.455 | 1.00 | 17.94 | 6 |
| ATOM | 13857 | O | LEU | G | 618 | 69.006 | 82.199 | 40.306 | 1.00 | 19.53 | 8 |
| ATOM | 13858 | N | LEU | G | 619 | 69.554 | 81.779 | 38.161 | 1.00 | 25.37 | 7 |
| ATOM | 13859 | CA | LEU | G | 619 | 68.243 | 82.075 | 37.611 | 1.00 | 25.49 | 6 |
| ATOM | 13860 | CB | LEU | G | 619 | 68.377 | 82.407 | 36.131 | 1.00 | 16.11 | 6 |
| ATOM | 13861 | CG | LEU | G | 619 | 68.183 | 83.825 | 35.641 | 1.00 | 13.87 | 6 |
| ATOM | 13862 | CD1 | LEU | G | 619 | 68.838 | 84.788 | 36.569 | 1.00 | 13.87 | 6 |
| ATOM | 13863 | CD2 | LEU | G | 619 | 68.759 | 83.921 | 34.251 | 1.00 | 13.87 | 6 |
| ATOM | 13864 | C | LEU | G | 619 | 67.362 | 80.832 | 37.773 | 1.00 | 28.24 | 6 |
| ATOM | 13865 | O | LEU | G | 619 | 67.528 | 80.055 | 38.712 | 1.00 | 28.33 | 8 |
| ATOM | 13866 | N | GLY | G | 620 | 66.442 | 80.637 | 36.835 | 1.00 | 107.94 | 7 |
| ATOM | 13867 | CA | GLY | G | 620 | 65.539 | 79.505 | 36.914 | 1.00 | 112.04 | 6 |
| ATOM | 13868 | C | GLY | G | 620 | 66.060 | 78.147 | 36.482 | 1.00 | 113.20 | 6 |
| ATOM | 13869 | O | GLY | G | 620 | 67.268 | 77.925 | 36.364 | 1.00 | 113.21 | 8 |
| ATOM | 13870 | N | LYS | G | 621 | 65.109 | 77.241 | 36.261 | 1.00 | 78.17 | 7 |
| ATOM | 13871 | CA | LYS | G | 621 | 65.364 | 75.871 | 35.830 | 1.00 | 77.93 | 6 |
| ATOM | 13872 | CB | LYS | G | 621 | 66.348 | 75.179 | 36.783 | 1.00 | 46.54 | 6 |
| ATOM | 13873 | CG | LYS | G | 621 | 67.751 | 75.050 | 36.216 | 1.00 | 46.21 | 6 |
| ATOM | 13874 | CD | LYS | G | 621 | 68.566 | 73.982 | 36.926 | 1.00 | 45.82 | 6 |
| ATOM | 13875 | CE | LYS | G | 621 | 69.924 | 73.822 | 36.264 | 1.00 | 44.86 | 6 |
| ATOM | 13876 | NZ | LYS | G | 621 | 70.758 | 72.809 | 36.949 | 1.00 | 41.06 | 7 |
| ATOM | 13877 | C | LYS | G | 621 | 64.052 | 75.082 | 35.791 | 1.00 | 78.01 | 6 |
| ATOM | 13878 | O | LYS | G | 621 | 63.314 | 75.074 | 36.771 | 1.00 | 80.07 | 8 |
| ATOM | 13879 | N | ARG | G | 622 | 63.768 | 74.432 | 34.660 | 1.00 | 26.65 | 7 |
| ATOM | 13880 | CA | ARG | G | 622 | 62.557 | 73.627 | 34.492 | 1.00 | 25.79 | 6 |
| ATOM | 13881 | CB | ARG | G | 622 | 62.384 | 73.248 | 33.027 | 1.00 | 60.74 | 6 |
| ATOM | 13882 | CG | ARG | G | 622 | 61.900 | 74.378 | 32.171 | 1.00 | 61.77 | 6 |
| ATOM | 13883 | CD | ARG | G | 622 | 60.395 | 74.504 | 32.266 | 1.00 | 63.51 | 6 |
| ATOM | 13884 | NE | ARG | G | 622 | 59.940 | 75.826 | 31.840 | 1.00 | 65.64 | 7 |
| ATOM | 13885 | CZ | ARG | G | 622 | 58.665 | 76.213 | 31.786 | 1.00 | 66.18 | 6 |
| ATOM | 13886 | NH1 | ARG | G | 622 | 57.684 | 75.381 | 32.122 | 1.00 | 64.18 | 7 |
| ATOM | 13887 | NH2 | ARG | G | 622 | 58.373 | 77.456 | 31.426 | 1.00 | 67.73 | 7 |
| ATOM | 13888 | C | ARG | G | 622 | 62.524 | 72.351 | 35.348 | 1.00 | 25.89 | 6 |
| ATOM | 13889 | O | ARG | G | 622 | 62.917 | 71.275 | 34.903 | 1.00 | 26.61 | 8 |
| ATOM | 13890 | N | VAL | G | 623 | 62.044 | 72.478 | 36.581 | 1.00 | 20.79 | 7 |
| ATOM | 13891 | CA | VAL | G | 623 | 61.940 | 71.349 | 37.495 | 1.00 | 21.50 | 6 |
| ATOM | 13892 | CB | VAL | G | 623 | 61.307 | 71.780 | 38.828 | 1.00 | 94.77 | 6 |
| ATOM | 13893 | CG1 | VAL | G | 623 | 61.294 | 70.611 | 39.802 | 1.00 | 96.54 | 6 |
| ATOM | 13894 | CG2 | VAL | G | 623 | 62.084 | 72.948 | 39.407 | 1.00 | 95.05 | 6 |
| ATOM | 13895 | C | VAL | G | 623 | 61.096 | 70.232 | 36.871 | 1.00 | 21.04 | 6 |
| ATOM | 13896 | O | VAL | G | 623 | 60.007 | 70.471 | 36.341 | 1.00 | 19.07 | 8 |
| ATOM | 13897 | N | ASP | G | 624 | 61.610 | 69.010 | 36.979 | 1.00 | 45.47 | 7 |
| ATOM | 13898 | CA | ASP | G | 624 | 60.997 | 67.819 | 36.406 | 1.00 | 45.30 | 6 |
| ATOM | 13899 | CB | ASP | G | 624 | 62.107 | 66.839 | 36.010 | 1.00 | 59.99 | 6 |
| ATOM | 13900 | CG | ASP | G | 624 | 61.654 | 65.816 | 34.988 | 1.00 | 59.38 | 6 |
| ATOM | 13901 | OD1 | ASP | G | 624 | 60.913 | 66.199 | 34.060 | 1.00 | 58.91 | 8 |
| ATOM | 13902 | OD2 | ASP | G | 624 | 62.055 | 64.636 | 35.101 | 1.00 | 59.47 | 8 |
| ATOM | 13903 | C | ASP | G | 624 | 59.988 | 67.094 | 37.275 | 1.00 | 45.22 | 6 |
| ATOM | 13904 | O | ASP | G | 624 | 59.806 | 65.895 | 37.118 | 1.00 | 46.96 | 8 |
| ATOM | 13905 | N | TYR | G | 625 | 59.323 | 67.794 | 38.186 | 1.00 | 49.99 | 7 |
| ATOM | 13906 | CA | TYR | G | 625 | 58.331 | 67.120 | 39.027 | 1.00 | 49.16 | 6 |
| ATOM | 13907 | CB | TYR | G | 625 | 59.020 | 66.346 | 40.148 | 1.00 | 33.14 | 6 |
| ATOM | 13908 | CG | TYR | G | 625 | 59.310 | 64.887 | 39.856 | 1.00 | 29.99 | 6 |
| ATOM | 13909 | CD1 | TYR | G | 625 | 60.620 | 64.449 | 39.681 | 1.00 | 29.37 | 6 |
| ATOM | 13910 | CE1 | TYR | G | 625 | 60.912 | 63.115 | 39.507 | 1.00 | 27.99 | 6 |
| ATOM | 13911 | CD2 | TYR | G | 625 | 58.288 | 63.935 | 39.841 | 1.00 | 27.42 | 6 |
| ATOM | 13912 | CE2 | TYR | G | 625 | 58.573 | 62.593 | 39.674 | 1.00 | 26.81 | 6 |
| ATOM | 13913 | CZ | TYR | G | 625 | 59.895 | 62.193 | 39.510 | 1.00 | 27.31 | 6 |
| ATOM | 13914 | OH | TYR | G | 625 | 60.233 | 60.872 | 39.368 | 1.00 | 28.32 | 8 |
| ATOM | 13915 | C | TYR | G | 625 | 57.261 | 68.009 | 39.658 | 1.00 | 49.98 | 6 |
| ATOM | 13916 | O | TYR | G | 625 | 56.832 | 67.743 | 40.787 | 1.00 | 49.63 | 8 |
| ATOM | 13917 | N | SER | G | 626 | 56.808 | 69.040 | 38.948 | 1.00 | 52.41 | 7 |
| ATOM | 13918 | CA | SER | G | 626 | 55.807 | 69.920 | 39.529 | 1.00 | 52.33 | 6 |
| ATOM | 13919 | CB | SER | G | 626 | 56.484 | 71.106 | 40.215 | 1.00 | 124.14 | 6 |
| ATOM | 13920 | OG | SER | G | 626 | 55.540 | 71.873 | 40.944 | 1.00 | 125.46 | 8 |
| ATOM | 13921 | C | SER | G | 626 | 54.775 | 70.444 | 38.563 | 1.00 | 51.15 | 6 |
| ATOM | 13922 | O | SER | G | 626 | 54.878 | 70.244 | 37.359 | 1.00 | 51.25 | 8 |
| ATOM | 13923 | N | GLY | G | 627 | 53.783 | 71.134 | 39.121 | 1.00 | 58.09 | 7 |
| ATOM | 13924 | CA | GLY | G | 627 | 52.710 | 71.707 | 38.331 | 1.00 | 56.01 | 6 |
| ATOM | 13925 | C | GLY | G | 627 | 52.141 | 72.981 | 38.927 | 1.00 | 53.66 | 6 |
| ATOM | 13926 | O | GLY | G | 627 | 52.675 | 73.541 | 39.878 | 1.00 | 53.41 | 8 |
| ATOM | 13927 | N | ARG | G | 628 | 51.037 | 73.437 | 38.357 | 1.00 | 28.82 | 7 |
| ATOM | 13928 | CA | ARG | G | 628 | 50.394 | 74.659 | 38.801 | 1.00 | 27.47 | 6 |
| ATOM | 13929 | CB | ARG | G | 628 | 50.950 | 75.862 | 38.032 | 1.00 | 53.79 | 6 |
| ATOM | 13930 | CG | ARG | G | 628 | 52.416 | 75.756 | 37.635 | 1.00 | 57.63 | 6 |
| ATOM | 13931 | CD | ARG | G | 628 | 52.876 | 76.935 | 36.772 | 1.00 | 59.23 | 6 |
| ATOM | 13932 | NE | ARG | G | 628 | 51.971 | 77.215 | 35.659 | 1.00 | 61.60 | 7 |
| ATOM | 13933 | CZ | ARG | G | 628 | 50.918 | 78.028 | 35.735 | 1.00 | 64.00 | 6 |
| ATOM | 13934 | NH1 | ARG | G | 628 | 50.635 | 78.645 | 36.878 | 1.00 | 64.19 | 7 |
| ATOM | 13935 | NH2 | ARG | G | 628 | 50.146 | 78.234 | 34.668 | 1.00 | 63.58 | 7 |
| ATOM | 13936 | C | ARG | G | 628 | 48.898 | 74.557 | 38.531 | 1.00 | 26.02 | 6 |
| ATOM | 13937 | O | ARG | G | 628 | 48.424 | 73.582 | 37.954 | 1.00 | 25.58 | 8 |
| ATOM | 13938 | N | SER | G | 629 | 48.163 | 75.579 | 38.950 | 1.00 | 30.56 | 7 |
| ATOM | 13939 | CA | SER | G | 629 | 46.726 | 75.633 | 38.755 | 1.00 | 28.99 | 6 |
| ATOM | 13940 | CB | SER | G | 629 | 46.059 | 74.344 | 39.213 | 1.00 | 43.18 | 6 |
| ATOM | 13941 | OG | SER | G | 629 | 44.649 | 74.481 | 39.162 | 1.00 | 45.83 | 8 |
| ATOM | 13942 | C | SER | G | 629 | 46.162 | 76.764 | 39.565 | 1.00 | 26.79 | 6 |
| ATOM | 13943 | O | SER | G | 629 | 46.817 | 77.269 | 40.470 | 1.00 | 27.02 | 8 |
| ATOM | 13944 | N | VAL | G | 630 | 44.942 | 77.162 | 39.239 | 1.00 | 14.17 | 7 |
| ATOM | 13945 | CA | VAL | G | 630 | 44.283 | 78.218 | 39.977 | 1.00 | 14.24 | 6 |
| ATOM | 13946 | CB | VAL | G | 630 | 42.881 | 78.447 | 39.396 | 1.00 | 13.87 | 6 |
| ATOM | 13947 | CG1 | VAL | G | 630 | 41.960 | 79.043 | 40.417 | 1.00 | 13.87 | 6 |
| ATOM | 13948 | CG2 | VAL | G | 630 | 42.987 | 79.365 | 38.203 | 1.00 | 13.87 | 6 |
| ATOM | 13949 | C | VAL | G | 630 | 44.227 | 77.752 | 41.435 | 1.00 | 15.94 | 6 |
| ATOM | 13950 | O | VAL | G | 630 | 44.435 | 76.582 | 41.714 | 1.00 | 16.30 | 8 |
| ATOM | 13951 | N | ILE | G | 631 | 43.999 | 78.646 | 42.380 | 1.00 | 35.03 | 7 |
| ATOM | 13952 | CA | ILE | G | 631 | 43.931 | 78.198 | 43.760 | 1.00 | 37.30 | 6 |
| ATOM | 13953 | CB | ILE | G | 631 | 45.077 | 78.747 | 44.620 | 1.00 | 51.58 | 6 |
| ATOM | 13954 | CG2 | ILE | G | 631 | 46.310 | 77.898 | 44.424 | 1.00 | 52.75 | 6 |
| ATOM | 13955 | CG1 | ILE | G | 631 | 45.300 | 80.226 | 44.302 | 1.00 | 53.75 | 6 |
| ATOM | 13956 | CD1 | ILE | G | 631 | 46.234 | 80.919 | 45.255 | 1.00 | 54.63 | 6 |
| ATOM | 13957 | C | ILE | G | 631 | 42.640 | 78.653 | 44.374 | 1.00 | 35.86 | 6 |
| ATOM | 13958 | O | ILE | G | 631 | 42.140 | 79.732 | 44.036 | 1.00 | 37.41 | 8 |
| ATOM | 13959 | N | VAL | G | 632 | 42.104 | 77.813 | 45.263 | 1.00 | 35.48 | 7 |
| ATOM | 13960 | CA | VAL | G | 632 | 40.862 | 78.088 | 45.969 | 1.00 | 33.73 | 6 |
| ATOM | 13961 | CB | VAL | G | 632 | 39.722 | 77.190 | 45.505 | 1.00 | 13.87 | 6 |
| ATOM | 13962 | CG1 | VAL | G | 632 | 38.556 | 77.398 | 46.410 | 1.00 | 13.87 | 6 |
| ATOM | 13963 | CG2 | VAL | G | 632 | 39.321 | 77.518 | 44.083 | 1.00 | 13.87 | 6 |
| ATOM | 13964 | C | VAL | G | 632 | 41.019 | 77.870 | 47.464 | 1.00 | 34.15 | 6 |
| ATOM | 13965 | O | VAL | G | 632 | 41.772 | 76.997 | 47.901 | 1.00 | 34.87 | 8 |
| ATOM | 13966 | N | VAL | G | 633 | 40.305 | 78.672 | 48.250 | 1.00 | 15.13 | 7 |
| ATOM | 13967 | CA | VAL | G | 633 | 40.366 | 78.548 | 49.696 | 1.00 | 14.56 | 6 |
| ATOM | 13968 | CB | VAL | G | 633 | 39.321 | 79.416 | 50.374 | 1.00 | 17.98 | 6 |
| ATOM | 13969 | CG1 | VAL | G | 633 | 37.940 | 79.010 | 49.921 | 1.00 | 17.51 | 6 |
| ATOM | 13970 | CG2 | VAL | G | 633 | 39.435 | 79.267 | 51.866 | 1.00 | 18.40 | 6 |
| ATOM | 13971 | C | VAL | G | 633 | 40.072 | 77.103 | 50.026 | 1.00 | 15.01 | 6 |
| ATOM | 13972 | O | VAL | G | 633 | 39.540 | 76.383 | 49.199 | 1.00 | 14.73 | 8 |
| ATOM | 13973 | N | GLY | G | 634 | 40.410 | 76.672 | 51.226 | 1.00 | 24.13 | 7 |
| ATOM | 13974 | CA | GLY | G | 634 | 40.144 | 75.296 | 51.569 | 1.00 | 26.32 | 6 |
| ATOM | 13975 | C | GLY | G | 634 | 40.249 | 74.959 | 53.037 | 1.00 | 27.61 | 6 |
| ATOM | 13976 | O | GLY | G | 634 | 41.319 | 74.568 | 53.506 | 1.00 | 27.49 | 8 |
| ATOM | 13977 | N | PRO | G | 635 | 39.155 | 75.118 | 53.798 | 1.00 | 34.42 | 7 |
| ATOM | 13978 | CD | PRO | G | 635 | 37.874 | 75.757 | 53.444 | 1.00 | 14.50 | 6 |
| ATOM | 13979 | CA | PRO | G | 635 | 39.215 | 74.789 | 55.225 | 1.00 | 35.61 | 6 |
| ATOM | 13980 | CB | PRO | G | 635 | 37.843 | 75.213 | 55.733 | 1.00 | 16.25 | 6 |
| ATOM | 13981 | CG | PRO | G | 635 | 37.421 | 76.294 | 54.753 | 1.00 | 15.80 | 6 |
| ATOM | 13982 | C | PRO | G | 635 | 39.464 | 73.274 | 55.362 | 1.00 | 39.07 | 6 |
| ATOM | 13983 | O | PRO | G | 635 | 38.534 | 72.488 | 55.602 | 1.00 | 38.33 | 8 |
| ATOM | 13984 | N | GLN | G | 636 | 40.727 | 72.883 | 55.169 | 1.00 | 59.89 | 7 |
| ATOM | 13985 | CA | GLN | G | 636 | 41.177 | 71.489 | 55.246 | 1.00 | 60.82 | 6 |
| ATOM | 13986 | CB | GLN | G | 636 | 42.413 | 71.277 | 54.381 | 1.00 | 49.54 | 6 |
| ATOM | 13987 | CG | GLN | G | 636 | 42.223 | 71.510 | 52.915 | 1.00 | 51.17 | 6 |
| ATOM | 13988 | CD | GLN | G | 636 | 43.479 | 72.062 | 52.285 | 1.00 | 52.97 | 6 |
| ATOM | 13989 | OE1 | GLN | G | 636 | 43.709 | 73.264 | 52.309 | 1.00 | 53.14 | 8 |
| ATOM | 13990 | NE2 | GLN | G | 636 | 44.314 | 71.186 | 51.741 | 1.00 | 54.60 | 7 |
| ATOM | 13991 | C | GLN | G | 636 | 41.559 | 71.168 | 56.676 | 1.00 | 61.13 | 6 |
| ATOM | 13992 | O | GLN | G | 636 | 40.742 | 71.279 | 57.584 | 1.00 | 61.46 | 8 |
| ATOM | 13993 | N | ALA | G | 637 | 42.811 | 70.761 | 56.869 | 1.00 | 53.10 | 7 |
| ATOM | 13994 | CA | ALA | G | 637 | 43.302 | 70.440 | 58.203 | 1.00 | 54.21 | 6 |
| ATOM | 13995 | CB | ALA | G | 637 | 44.026 | 69.113 | 58.213 | 1.00 | 71.85 | 6 |
| ATOM | 13996 | C | ALA | G | 637 | 44.240 | 71.539 | 58.624 | 1.00 | 53.34 | 6 |
| ATOM | 13997 | O | ALA | G | 637 | 45.093 | 71.333 | 59.483 | 1.00 | 52.33 | 8 |
| ATOM | 13998 | N | ALA | G | 638 | 44.054 | 72.702 | 58.000 | 1.00 | 49.90 | 7 |
| ATOM | 13999 | CA | ALA | G | 638 | 44.839 | 73.899 | 58.266 | 1.00 | 48.08 | 6 |
| ATOM | 14000 | CB | ALA | G | 638 | 44.380 | 74.565 | 59.527 | 1.00 | 33.63 | 6 |
| ATOM | 14001 | C | ALA | G | 638 | 46.306 | 73.608 | 58.367 | 1.00 | 47.42 | 6 |
| ATOM | 14002 | O | ALA | G | 638 | 46.733 | 72.732 | 59.113 | 1.00 | 46.58 | 8 |
| ATOM | 14003 | N | LEU | G | 639 | 47.092 | 74.368 | 57.620 | 1.00 | 56.66 | 7 |
| ATOM | 14004 | CA | LEU | G | 639 | 48.519 | 74.155 | 57.615 | 1.00 | 54.41 | 6 |
| ATOM | 14005 | CB | LEU | G | 639 | 49.089 | 74.390 | 59.011 | 1.00 | 13.87 | 6 |
| ATOM | 14006 | CG | LEU | G | 639 | 48.386 | 75.393 | 59.912 | 1.00 | 13.87 | 6 |
| ATOM | 14007 | CD1 | LEU | G | 639 | 49.148 | 75.508 | 61.198 | 1.00 | 13.87 | 6 |
| ATOM | 14008 | CD2 | LEU | G | 639 | 48.315 | 76.723 | 59.235 | 1.00 | 13.87 | 6 |
| ATOM | 14009 | C | LEU | G | 639 | 48.540 | 72.674 | 57.314 | 1.00 | 55.55 | 6 |
| ATOM | 14010 | O | LEU | G | 639 | 47.502 | 72.089 | 56.986 | 1.00 | 55.99 | 8 |
| ATOM | 14011 | N | HIS | G | 640 | 49.706 | 72.060 | 57.393 | 1.00 | 47.02 | 7 |
| ATOM | 14012 | CA | HIS | G | 640 | 49.743 | 70.634 | 57.198 | 1.00 | 47.70 | 6 |
| ATOM | 14013 | CB | HIS | G | 640 | 48.840 | 70.027 | 58.257 | 1.00 | 53.49 | 6 |
| ATOM | 14014 | CG | HIS | G | 640 | 49.482 | 68.950 | 59.045 | 1.00 | 57.32 | 6 |
| ATOM | 14015 | CD2 | HIS | G | 640 | 48.960 | 67.866 | 59.659 | 1.00 | 58.54 | 6 |
| ATOM | 14016 | ND1 | HIS | G | 640 | 50.829 | 68.946 | 59.325 | 1.00 | 60.71 | 7 |
| ATOM | 14017 | CE1 | HIS | G | 640 | 51.111 | 67.902 | 60.081 | 1.00 | 62.34 | 6 |
| ATOM | 14018 | NE2 | HIS | G | 640 | 49.993 | 67.231 | 60.300 | 1.00 | 61.91 | 7 |
| ATOM | 14019 | C | HIS | G | 640 | 49.311 | 70.108 | 55.828 | 1.00 | 47.48 | 6 |
| ATOM | 14020 | O | HIS | G | 640 | 50.120 | 69.599 | 55.058 | 1.00 | 47.90 | 8 |
| ATOM | 14021 | N | GLN | G | 641 | 48.023 | 70.244 | 55.535 | 1.00 | 31.89 | 7 |
| ATOM | 14022 | CA | GLN | G | 641 | 47.448 | 69.696 | 54.324 | 1.00 | 30.23 | 6 |
| ATOM | 14023 | CB | GLN | G | 641 | 46.045 | 69.189 | 54.637 | 1.00 | 28.09 | 6 |
| ATOM | 14024 | CG | GLN | G | 641 | 46.019 | 68.435 | 55.958 | 1.00 | 26.62 | 6 |
| ATOM | 14025 | CD | GLN | G | 641 | 45.055 | 67.280 | 55.975 | 1.00 | 25.29 | 6 |
| ATOM | 14026 | OE1 | GLN | G | 641 | 43.835 | 67.465 | 55.888 | 1.00 | 23.03 | 8 |
| ATOM | 14027 | NE2 | GLN | G | 641 | 45.599 | 66.066 | 56.087 | 1.00 | 24.47 | 7 |
| ATOM | 14028 | C | GLN | G | 641 | 47.429 | 70.535 | 53.078 | 1.00 | 30.20 | 6 |
| ATOM | 14029 | O | GLN | G | 641 | 47.674 | 71.738 | 53.113 | 1.00 | 30.30 | 8 |
| ATOM | 14030 | N | CYS | G | 642 | 47.105 | 69.869 | 51.972 | 1.00 | 59.16 | 7 |
| ATOM | 14031 | CA | CYS | G | 642 | 47.092 | 70.486 | 50.658 | 1.00 | 59.05 | 6 |
| ATOM | 14032 | CB | CYS | G | 642 | 48.428 | 70.189 | 49.991 | 1.00 | 39.80 | 6 |
| ATOM | 14033 | SG | CYS | G | 642 | 48.944 | 71.434 | 48.854 | 1.00 | 42.08 | 16 |
| ATOM | 14034 | C | CYS | G | 642 | 45.960 | 69.971 | 49.770 | 1.00 | 58.19 | 6 |
| ATOM | 14035 | O | CYS | G | 642 | 45.579 | 68.802 | 49.855 | 1.00 | 59.53 | 8 |
| ATOM | 14036 | N | GLY | G | 643 | 45.433 | 70.847 | 48.917 | 1.00 | 30.01 | 7 |
| ATOM | 14037 | CA | GLY | G | 643 | 44.366 | 70.460 | 48.005 | 1.00 | 27.94 | 6 |
| ATOM | 14038 | C | GLY | G | 643 | 44.874 | 69.424 | 47.023 | 1.00 | 27.01 | 6 |
| ATOM | 14039 | O | GLY | G | 643 | 44.934 | 68.245 | 47.357 | 1.00 | 28.10 | 8 |
| ATOM | 14040 | N | LEU | G | 644 | 45.235 | 69.847 | 45.815 | 1.00 | 36.37 | 7 |
| ATOM | 14041 | CA | LEU | G | 644 | 45.778 | 68.933 | 44.802 | 1.00 | 36.23 | 6 |
| ATOM | 14042 | CB | LEU | G | 644 | 46.899 | 68.069 | 45.398 | 1.00 | 16.15 | 6 |
| ATOM | 14043 | CG | LEU | G | 644 | 48.324 | 68.376 | 44.937 | 1.00 | 13.87 | 6 |
| ATOM | 14044 | CD1 | LEU | G | 644 | 49.308 | 67.599 | 45.794 | 1.00 | 13.87 | 6 |
| ATOM | 14045 | CD2 | LEU | G | 644 | 48.482 | 68.043 | 43.460 | 1.00 | 13.87 | 6 |
| ATOM | 14046 | C | LEU | G | 644 | 44.791 | 68.005 | 44.114 | 1.00 | 37.17 | 6 |
| ATOM | 14047 | O | LEU | G | 644 | 44.489 | 66.922 | 44.619 | 1.00 | 38.29 | 8 |
| ATOM | 14048 | N | PRO | G | 645 | 44.302 | 68.403 | 42.930 | 1.00 | 27.77 | 7 |
| ATOM | 14049 | CD | PRO | G | 645 | 44.726 | 69.591 | 42.172 | 1.00 | 22.15 | 6 |
| ATOM | 14050 | CA | PRO | G | 645 | 43.345 | 67.599 | 42.161 | 1.00 | 27.56 | 6 |
| ATOM | 14051 | CB | PRO | G | 645 | 43.260 | 68.345 | 40.834 | 1.00 | 22.30 | 6 |
| ATOM | 14052 | CG | PRO | G | 645 | 43.615 | 69.759 | 41.198 | 1.00 | 22.67 | 6 |
| ATOM | 14053 | C | PRO | G | 645 | 43.941 | 66.218 | 41.980 | 1.00 | 28.10 | 6 |
| ATOM | 14054 | O | PRO | G | 645 | 44.967 | 66.093 | 41.333 | 1.00 | 28.41 | 8 |
| ATOM | 14055 | N | LYS | G | 646 | 43.329 | 65.178 | 42.533 | 1.00 | 30.15 | 7 |
| ATOM | 14056 | CA | LYS | G | 646 | 43.910 | 63.842 | 42.383 | 1.00 | 30.47 | 6 |
| ATOM | 14057 | CB | LYS | G | 646 | 42.888 | 62.755 | 42.741 | 1.00 | 24.99 | 6 |
| ATOM | 14058 | CG | LYS | G | 646 | 41.577 | 62.860 | 42.009 | 1.00 | 23.28 | 6 |
| ATOM | 14059 | CD | LYS | G | 646 | 40.559 | 61.945 | 42.637 | 1.00 | 23.51 | 6 |
| ATOM | 14060 | CE | LYS | G | 646 | 39.182 | 62.205 | 42.066 | 1.00 | 24.35 | 6 |
| ATOM | 14061 | NZ | LYS | G | 646 | 38.152 | 61.480 | 42.848 | 1.00 | 26.45 | 7 |
| ATOM | 14062 | C | LYS | G | 646 | 44.448 | 63.624 | 40.973 | 1.00 | 31.88 | 6 |
| ATOM | 14063 | O | LYS | G | 646 | 45.506 | 63.021 | 40.792 | 1.00 | 32.03 | 8 |
| ATOM | 14064 | N | ARG | G | 647 | 43.739 | 64.145 | 39.976 | 1.00 | 25.35 | 7 |
| ATOM | 14065 | CA | ARG | G | 647 | 44.180 | 63.983 | 38.597 | 1.00 | 26.83 | 6 |
| ATOM | 14066 | CB | ARG | G | 647 | 43.302 | 64.797 | 37.648 | 1.00 | 127.24 | 6 |
| ATOM | 14067 | CG | ARG | G | 647 | 43.458 | 64.345 | 36.210 | 1.00 | 133.86 | 6 |
| ATOM | 14068 | CD | ARG | G | 647 | 43.269 | 62.826 | 36.126 | 1.00 | 139.18 | 6 |
| ATOM | 14069 | NE | ARG | G | 647 | 43.940 | 62.228 | 34.975 | 1.00 | 144.06 | 7 |
| ATOM | 14070 | CZ | ARG | G | 647 | 44.091 | 60.918 | 34.801 | 1.00 | 145.06 | 6 |
| ATOM | 14071 | NH1 | ARG | G | 647 | 43.615 | 60.068 | 35.704 | 1.00 | 144.69 | 7 |
| ATOM | 14072 | NH2 | ARG | G | 647 | 44.722 | 60.456 | 33.729 | 1.00 | 145.29 | 7 |
| ATOM | 14073 | C | ARG | G | 647 | 45.640 | 64.388 | 38.438 | 1.00 | 25.31 | 6 |
| ATOM | 14074 | O | ARG | G | 647 | 46.417 | 63.690 | 37.793 | 1.00 | 25.84 | 8 |
| ATOM | 14075 | N | MET | G | 648 | 46.010 | 65.512 | 39.037 | 1.00 | 19.05 | 7 |
| ATOM | 14076 | CA | MET | G | 648 | 47.380 | 65.981 | 38.978 | 1.00 | 18.02 | 6 |
| ATOM | 14077 | CB | MET | G | 648 | 47.435 | 67.497 | 39.054 | 1.00 | 32.50 | 6 |
| ATOM | 14078 | CG | MET | G | 648 | 47.380 | 68.028 | 40.455 | 1.00 | 34.91 | 6 |
| ATOM | 14079 | SD | MET | G | 648 | 47.480 | 69.798 | 40.456 | 1.00 | 36.66 | 16 |
| ATOM | 14080 | CE | MET | G | 648 | 49.209 | 70.022 | 40.793 | 1.00 | 37.49 | 6 |
| ATOM | 14081 | C | MET | G | 648 | 48.217 | 65.396 | 40.120 | 1.00 | 17.12 | 6 |
| ATOM | 14082 | O | MET | G | 648 | 49.436 | 65.534 | 40.139 | 1.00 | 15.88 | 8 |
| ATOM | 14083 | N | ALA | G | 649 | 47.581 | 64.761 | 41.093 | 1.00 | 34.65 | 7 |
| ATOM | 14084 | CA | ALA | G | 649 | 48.363 | 64.183 | 42.173 | 1.00 | 35.55 | 6 |
| ATOM | 14085 | CB | ALA | G | 649 | 47.471 | 63.766 | 43.335 | 1.00 | 20.55 | 6 |
| ATOM | 14086 | C | ALA | G | 649 | 48.974 | 62.974 | 41.520 | 1.00 | 36.05 | 6 |
| ATOM | 14087 | O | ALA | G | 649 | 50.156 | 62.685 | 41.696 | 1.00 | 36.72 | 8 |
| ATOM | 14088 | N | LEU | G | 650 | 48.145 | 62.297 | 40.729 | 1.00 | 43.51 | 7 |
| ATOM | 14089 | CA | LEU | G | 650 | 48.526 | 61.079 | 40.013 | 1.00 | 44.21 | 6 |
| ATOM | 14090 | CB | LEU | G | 650 | 47.339 | 60.567 | 39.185 | 1.00 | 54.68 | 6 |
| ATOM | 14091 | CG | LEU | G | 650 | 47.436 | 59.214 | 38.477 | 1.00 | 54.88 | 6 |
| ATOM | 14092 | CD1 | LEU | G | 650 | 46.947 | 58.101 | 39.388 | 1.00 | 55.39 | 6 |
| ATOM | 14093 | CD2 | LEU | G | 650 | 46.585 | 59.268 | 37.228 | 1.00 | 55.01 | 6 |
| ATOM | 14094 | C | LEU | G | 650 | 49.715 | 61.331 | 39.096 | 1.00 | 44.70 | 6 |
| ATOM | 14095 | O | LEU | G | 650 | 50.597 | 60.482 | 38.948 | 1.00 | 44.72 | 8 |
| ATOM | 14096 | N | GLU | G | 651 | 49.728 | 62.500 | 38.470 | 1.00 | 46.93 | 7 |
| ATOM | 14097 | CA | GLU | G | 651 | 50.819 | 62.826 | 37.584 | 1.00 | 46.42 | 6 |
| ATOM | 14098 | CB | GLU | G | 651 | 50.515 | 64.085 | 36.792 | 1.00 | 60.92 | 6 |
| ATOM | 14099 | CG | GLU | G | 651 | 50.570 | 63.858 | 35.301 | 1.00 | 65.33 | 6 |
| ATOM | 14100 | CD | GLU | G | 651 | 51.733 | 62.972 | 34.901 | 1.00 | 67.82 | 6 |
| ATOM | 14101 | OE1 | GLU | G | 651 | 52.850 | 63.195 | 35.415 | 1.00 | 70.14 | 8 |
| ATOM | 14102 | OE2 | GLU | G | 651 | 51.534 | 62.060 | 34.069 | 1.00 | 67.84 | 8 |
| ATOM | 14103 | C | GLU | C | 651 | 52.053 | 63.039 | 38.426 | 1.00 | 45.52 | 6 |
| ATOM | 14104 | O | GLU | G | 651 | 52.995 | 62.259 | 38.366 | 1.00 | 45.96 | 8 |
| ATOM | 14105 | N | LEU | G | 652 | 52.026 | 64.091 | 39.233 | 1.00 | 36.11 | 7 |
| ATOM | 14106 | CA | LEU | G | 652 | 53.147 | 64.423 | 40.102 | 1.00 | 34.41 | 6 |
| ATOM | 14107 | CB | LEU | G | 652 | 52.710 | 65.446 | 41.168 | 1.00 | 21.60 | 6 |
| ATOM | 14108 | CG | LEU | G | 652 | 52.664 | 66.925 | 40.773 | 1.00 | 21.36 | 6 |
| ATOM | 14109 | CD1 | LEU | G | 652 | 54.037 | 67.366 | 40.270 | 1.00 | 19.67 | 6 |
| ATOM | 14110 | CD2 | LEU | G | 652 | 51.638 | 67.132 | 39.688 | 1.00 | 22.23 | 6 |
| ATOM | 14111 | C | LEU | G | 652 | 53.806 | 63.234 | 40.795 | 1.00 | 33.17 | 6 |
| ATOM | 14112 | O | LEU | G | 652 | 54.954 | 63.332 | 41.219 | 1.00 | 32.50 | 8 |
| ATOM | 14113 | N | PHE | G | 653 | 53.098 | 62.114 | 40.905 | 1.00 | 68.11 | 7 |
| ATOM | 14114 | CA | PHE | G | 653 | 53.654 | 60.952 | 41.598 | 1.00 | 68.09 | 6 |
| ATOM | 14115 | CB | PHE | G | 653 | 52.853 | 60.704 | 42.897 | 1.00 | 15.11 | 6 |
| ATOM | 14116 | CG | PHE | G | 653 | 53.155 | 61.683 | 44.012 | 1.00 | 13.87 | 6 |
| ATOM | 14117 | CD1 | PHE | G | 653 | 53.081 | 63.056 | 43.805 | 1.00 | 13.87 | 6 |
| ATOM | 14118 | CD2 | PHE | G | 653 | 53.494 | 61.226 | 45.274 | 1.00 | 13.87 | 6 |
| ATOM | 14119 | CE1 | PHE | G | 653 | 53.339 | 63.950 | 44.833 | 1.00 | 13.87 | 6 |
| ATOM | 14120 | CE2 | PHE | G | 653 | 53.750 | 62.117 | 46.304 | 1.00 | 13.87 | 6 |
| ATOM | 14121 | CZ | PHE | G | 653 | 53.670 | 63.481 | 46.079 | 1.00 | 13.87 | 6 |
| ATOM | 14122 | C | PHE | G | 653 | 53.758 | 59.630 | 40.811 | 1.00 | 69.20 | 6 |
| ATOM | 14123 | O | PHE | G | 653 | 54.442 | 58.705 | 41.263 | 1.00 | 72.31 | 8 |
| ATOM | 14124 | N | LYS | G | 654 | 53.117 | 59.547 | 39.641 | 1.00 | 18.77 | 7 |
| ATOM | 14125 | CA | LYS | G | 654 | 53.106 | 58.308 | 38.851 | 1.00 | 15.87 | 6 |
| ATOM | 14126 | CB | LYS | G | 654 | 53.074 | 58.574 | 37.345 | 1.00 | 23.37 | 6 |
| ATOM | 14127 | CG | LYS | G | 654 | 52.859 | 57.283 | 36.557 | 1.00 | 22.14 | 6 |
| ATOM | 14128 | CD | LYS | G | 654 | 52.856 | 57.484 | 35.048 | 1.00 | 19.84 | 6 |
| ATOM | 14129 | CE | LYS | G | 654 | 51.618 | 58.224 | 34.565 | 1.00 | 18.72 | 6 |
| ATOM | 14130 | NZ | LYS | G | 654 | 51.687 | 58.463 | 33.091 | 1.00 | 19.10 | 7 |
| ATOM | 14131 | C | LYS | G | 654 | 54.221 | 57.335 | 39.142 | 1.00 | 14.73 | 6 |
| ATOM | 14132 | O | LYS | G | 654 | 53.960 | 56.254 | 39.650 | 1.00 | 13.87 | 8 |
| ATOM | 14133 | N | PRO | G | 655 | 55.480 | 57.694 | 38.822 | 1.00 | 13.87 | 7 |
| ATOM | 14134 | CD | PRO | G | 655 | 56.032 | 58.955 | 38.299 | 1.00 | 22.00 | 6 |
| ATOM | 14135 | CA | PRO | G | 655 | 56.565 | 56.751 | 39.101 | 1.00 | 14.48 | 6 |
| ATOM | 14136 | CB | PRO | G | 655 | 57.804 | 57.640 | 39.090 | 1.00 | 21.20 | 6 |
| ATOM | 14137 | CG | PRO | G | 655 | 57.481 | 58.577 | 38.005 | 1.00 | 20.69 | 6 |
| ATOM | 14138 | C | PRO | G | 655 | 56.326 | 56.094 | 40.440 | 1.00 | 15.35 | 6 |
| ATOM | 14139 | O | PRO | G | 655 | 56.027 | 54.911 | 40.497 | 1.00 | 14.19 | 8 |
| ATOM | 14140 | N | PHE | G | 656 | 56.415 | 56.879 | 41.511 | 1.00 | 21.23 | 7 |
| ATOM | 14141 | CA | PHE | G | 656 | 56.203 | 56.351 | 42.851 | 1.00 | 24.52 | 6 |
| ATOM | 14142 | CB | PHE | G | 656 | 55.937 | 57.466 | 43.853 | 1.00 | 56.36 | 6 |
| ATOM | 14143 | CG | PHE | G | 656 | 57.114 | 58.341 | 44.084 | 1.00 | 57.81 | 6 |
| ATOM | 14144 | CD1 | PHE | G | 656 | 57.429 | 59.341 | 43.179 | 1.00 | 58.17 | 6 |
| ATOM | 14145 | CD2 | PHE | G | 656 | 57.948 | 58.127 | 45.172 | 1.00 | 57.86 | 6 |
| ATOM | 14146 | CE1 | PHE | G | 656 | 58.558 | 60.118 | 43.352 | 1.00 | 58.92 | 6 |
| ATOM | 14147 | CE2 | PHE | G | 656 | 59.079 | 58.899 | 45.356 | 1.00 | 59.09 | 6 |
| ATOM | 14148 | CZ | PHE | G | 656 | 59.389 | 59.899 | 44.441 | 1.00 | 59.84 | 6 |
| ATOM | 14149 | C | PHE | G | 656 | 55.035 | 55.410 | 42.822 | 1.00 | 25.71 | 6 |
| ATOM | 14150 | O | PHE | G | 656 | 55.071 | 54.385 | 43.494 | 1.00 | 25.94 | 8 |
| ATOM | 14151 | N | LEU | G | 657 | 54.002 | 55.757 | 42.054 | 1.00 | 55.43 | 7 |
| ATOM | 14152 | CA | LEU | G | 657 | 52.847 | 54.883 | 41.934 | 1.00 | 57.39 | 6 |
| ATOM | 14153 | CB | LEU | G | 657 | 51.721 | 55.534 | 41.131 | 1.00 | 35.88 | 6 |
| ATOM | 14154 | CG | LEU | G | 657 | 50.487 | 54.626 | 41.028 | 1.00 | 34.82 | 6 |
| ATOM | 14155 | CD1 | LEU | G | 657 | 49.888 | 54.480 | 42.397 | 1.00 | 33.46 | 6 |
| ATOM | 14156 | CD2 | LEU | G | 657 | 49.460 | 55.183 | 40.053 | 1.00 | 34.01 | 6 |
| ATOM | 14157 | C | LEU | G | 657 | 53.354 | 53.634 | 41.217 | 1.00 | 59.67 | 6 |
| ATOM | 14158 | O | LEU | G | 657 | 53.951 | 52.782 | 41.860 | 1.00 | 60.86 | 8 |
| ATOM | 14159 | N | LEU | G | 658 | 53.150 | 53.530 | 39.901 | 1.00 | 45.66 | 7 |
| ATOM | 14160 | CA | LEU | G | 658 | 53.607 | 52.360 | 39.138 | 1.00 | 47.25 | 6 |
| ATOM | 14161 | CB | LEU | G | 658 | 54.691 | 52.748 | 38.139 | 1.00 | 42.38 | 6 |
| ATOM | 14162 | CG | LEU | G | 658 | 54.531 | 54.006 | 37.307 | 1.00 | 40.85 | 6 |
| ATOM | 14163 | CD1 | LEU | G | 658 | 55.761 | 54.173 | 36.405 | 1.00 | 40.90 | 6 |
| ATOM | 14164 | CD2 | LEU | G | 658 | 53.245 | 53.911 | 36.507 | 1.00 | 39.81 | 6 |
| ATOM | 14165 | C | LEU | G | 658 | 54.191 | 51.290 | 40.061 | 1.00 | 49.47 | 6 |
| ATOM | 14166 | O | LEU | G | 658 | 53.493 | 50.363 | 40.462 | 1.00 | 48.46 | 8 |
| ATOM | 14167 | N | LYS | G | 659 | 55.475 | 51.445 | 40.396 | 1.00 | 33.92 | 7 |
| ATOM | 14168 | CA | LYS | G | 659 | 56.189 | 50.524 | 41.266 | 1.00 | 37.81 | 6 |
| ATOM | 14169 | CB | LYS | G | 659 | 57.411 | 51.218 | 41.867 | 1.00 | 112.52 | 6 |
| ATOM | 14170 | CG | LYS | G | 659 | 58.279 | 50.319 | 42.729 | 1.00 | 117.06 | 6 |
| ATOM | 14171 | CD | LYS | G | 659 | 57.654 | 50.076 | 44.093 | 1.00 | 120.47 | 6 |
| ATOM | 14172 | CE | LYS | G | 659 | 58.485 | 49.122 | 44.934 | 1.00 | 122.21 | 6 |
| ATOM | 14173 | NZ | LYS | G | 659 | 57.930 | 48.986 | 46.311 | 1.00 | 123.50 | 7 |
| ATOM | 14174 | C | LYS | G | 659 | 55.259 | 50.032 | 42.369 | 1.00 | 39.05 | 6 |
| ATOM | 14175 | O | LYS | G | 659 | 55.185 | 48.829 | 42.647 | 1.00 | 39.17 | 8 |
| ATOM | 14176 | N | LYS | G | 660 | 54.553 | 50.964 | 43.002 | 1.00 | 78.10 | 7 |
| ATOM | 14177 | CA | LYS | G | 660 | 53.610 | 50.619 | 44.056 | 1.00 | 81.03 | 6 |
| ATOM | 14178 | CB | LYS | G | 660 | 52.731 | 51.817 | 44.409 | 1.00 | 102.85 | 6 |
| ATOM | 14179 | CG | LYS | G | 660 | 51.944 | 51.644 | 45.693 | 1.00 | 104.43 | 6 |
| ATOM | 14180 | CD | LYS | G | 660 | 52.879 | 51.492 | 46.885 | 1.00 | 105.45 | 6 |
| ATOM | 14181 | CE | LYS | G | 660 | 52.095 | 51.359 | 48.175 | 1.00 | 106.90 | 6 |
| ATOM | 14182 | NZ | LYS | G | 660 | 52.962 | 51.015 | 49.332 | 1.00 | 108.02 | 7 |
| ATOM | 14183 | C | LYS | G | 660 | 52.750 | 49.508 | 43.496 | 1.00 | 82.46 | 6 |
| ATOM | 14184 | O | LYS | G | 660 | 52.751 | 48.398 | 44.018 | 1.00 | 83.58 | 8 |
| ATOM | 14185 | N | MET | G | 661 | 52.034 | 49.815 | 42.418 | 1.00 | 73.78 | 7 |
| ATOM | 14186 | CA | MET | G | 661 | 51.177 | 48.844 | 41.744 | 1.00 | 74.70 | 6 |
| ATOM | 14187 | CB | MET | G | 661 | 50.676 | 49.408 | 40.422 | 1.00 | 47.67 | 6 |
| ATOM | 14188 | CG | MET | G | 661 | 49.745 | 50.573 | 40.583 | 1.00 | 43.82 | 6 |
| ATOM | 14189 | SD | MET | G | 661 | 49.083 | 51.056 | 39.019 | 1.00 | 37.36 | 16 |
| ATOM | 14190 | CE | MET | G | 661 | 50.450 | 51.991 | 38.440 | 1.00 | 37.75 | 6 |
| ATOM | 14191 | C | MET | G | 661 | 51.903 | 47.535 | 41.474 | 1.00 | 77.32 | 6 |
| ATOM | 14192 | O | MET | G | 661 | 51.379 | 46.462 | 41.767 | 1.00 | 77.89 | 8 |
| ATOM | 14193 | N | GLU | G | 662 | 53.098 | 47.626 | 40.895 | 1.00 | 41.30 | 7 |
| ATOM | 14194 | CA | GLU | G | 662 | 53.889 | 46.437 | 40.615 | 1.00 | 43.36 | 6 |
| ATOM | 14195 | CB | GLU | G | 662 | 55.353 | 46.787 | 40.326 | 1.00 | 97.38 | 6 |
| ATOM | 14196 | CG | GLU | G | 662 | 56.286 | 45.571 | 40.448 | 1.00 | 100.79 | 6 |
| ATOM | 14197 | CD | GLU | G | 662 | 57.748 | 45.930 | 40.708 | 1.00 | 102.66 | 6 |
| ATOM | 14198 | OE1 | GLU | G | 662 | 58.013 | 46.844 | 41.524 | 1.00 | 104.78 | 8 |
| ATOM | 14199 | OE2 | GLU | G | 662 | 58.637 | 45.278 | 40.111 | 1.00 | 102.38 | 8 |
| ATOM | 14200 | C | GLU | G | 662 | 53.848 | 45.561 | 41.850 | 1.00 | 44.11 | 6 |
| ATOM | 14201 | O | GLU | G | 662 | 53.162 | 44.541 | 41.883 | 1.00 | 44.20 | 8 |
| ATOM | 14202 | N | GLU | G | 663 | 54.575 | 45.981 | 42.876 | 1.00 | 79.97 | 7 |
| ATOM | 14203 | CA | GLU | G | 663 | 54.643 | 45.219 | 44.110 | 1.00 | 82.77 | 6 |
| ATOM | 14204 | CB | GLU | G | 663 | 55.610 | 45.896 | 45.082 | 1.00 | 185.83 | 6 |
| ATOM | 14205 | CG | GLU | G | 663 | 56.181 | 44.959 | 46.130 | 1.00 | 188.96 | 6 |
| ATOM | 14206 | CD | GLU | G | 663 | 57.317 | 45.585 | 46.905 | 1.00 | 190.76 | 6 |
| ATOM | 14207 | OE1 | GLU | G | 663 | 57.071 | 46.569 | 47.634 | 1.00 | 192.26 | 8 |
| ATOM | 14208 | OE2 | GLU | G | 663 | 58.459 | 45.096 | 46.779 | 1.00 | 191.84 | 8 |
| ATOM | 14209 | C | GLU | G | 663 | 53.267 | 45.063 | 44.747 | 1.00 | 83.00 | 6 |
| ATOM | 14210 | O | GLU | G | 663 | 52.996 | 44.079 | 45.445 | 1.00 | 82.12 | 8 |
| ATOM | 14211 | N | LYS | G | 664 | 52.391 | 46.027 | 44.491 | 1.00 | 72.63 | 7 |
| ATOM | 14212 | CA | LYS | G | 664 | 51.050 | 45.986 | 45.051 | 1.00 | 73.05 | 6 |
| ATOM | 14213 | CB | LYS | G | 664 | 50.298 | 47.278 | 44.742 | 1.00 | 76.35 | 6 |
| ATOM | 14214 | CG | LYS | G | 664 | 49.153 | 47.550 | 45.692 | 1.00 | 77.34 | 6 |
| ATOM | 14215 | CD | LYS | G | 664 | 49.665 | 47.664 | 47.107 | 1.00 | 78.12 | 6 |
| ATOM | 14216 | CE | LYS | G | 664 | 48.552 | 47.948 | 48.079 | 1.00 | 79.63 | 6 |
| ATOM | 14217 | NZ | LYS | G | 664 | 49.094 | 47.991 | 49.459 | 1.00 | 80.80 | 7 |
| ATOM | 14218 | C | LYS | G | 664 | 50.303 | 44.810 | 44.454 | 1.00 | 73.20 | 6 |
| ATOM | 14219 | O | LYS | G | 664 | 49.115 | 44.624 | 44.708 | 1.00 | 73.35 | 8 |
| ATOM | 14220 | N | ALA | G | 665 | 51.019 | 44.020 | 43.660 | 1.00 | 121.02 | 7 |
| ATOM | 14221 | CA | ALA | G | 665 | 50.463 | 42.848 | 42.996 | 1.00 | 121.79 | 6 |
| ATOM | 14222 | CB | ALA | G | 665 | 49.677 | 41.990 | 43.996 | 1.00 | 143.90 | 6 |
| ATOM | 14223 | C | ALA | G | 665 | 49.573 | 43.250 | 41.820 | 1.00 | 121.53 | 6 |
| ATOM | 14224 | O | ALA | G | 665 | 49.737 | 42.733 | 40.711 | 1.00 | 121.51 | 8 |
| ATOM | 14225 | N | PHE | G | 666 | 48.643 | 44.177 | 42.057 | 1.00 | 37.57 | 7 |
| ATOM | 14226 | CA | PHE | G | 666 | 47.746 | 44.619 | 41.000 | 1.00 | 37.28 | 6 |
| ATOM | 14227 | CB | PHE | G | 666 | 46.869 | 45.780 | 41.434 | 1.00 | 123.03 | 6 |
| ATOM | 14228 | CG | PHE | G | 666 | 46.038 | 46.307 | 40.321 | 1.00 | 125.47 | 6 |
| ATOM | 14229 | CD1 | PHE | G | 666 | 45.194 | 45.450 | 39.619 | 1.00 | 126.92 | 6 |
| ATOM | 14230 | CD2 | PHE | G | 666 | 46.150 | 47.623 | 39.910 | 1.00 | 126.53 | 6 |
| ATOM | 14231 | CE1 | PHE | G | 666 | 44.476 | 45.893 | 38.518 | 1.00 | 127.79 | 6 |
| ATOM | 14232 | CE2 | PHE | G | 666 | 45.435 | 48.079 | 38.806 | 1.00 | 127.42 | 6 |
| ATOM | 14233 | CZ | PHE | G | 666 | 44.595 | 47.209 | 38.108 | 1.00 | 127.68 | 6 |
| ATOM | 14234 | C | PHE | G | 666 | 48.520 | 45.049 | 39.780 | 1.00 | 36.21 | 6 |
| ATOM | 14235 | O | PHE | G | 666 | 49.528 | 45.743 | 39.894 | 1.00 | 35.65 | 8 |
| ATOM | 14236 | N | ALA | G | 667 | 48.024 | 44.652 | 38.615 | 1.00 | 71.70 | 7 |
| ATOM | 14237 | CA | ALA | G | 667 | 48.677 | 44.951 | 37.350 | 1.00 | 72.52 | 6 |
| ATOM | 14238 | CB | ALA | G | 667 | 49.100 | 46.416 | 37.295 | 1.00 | 56.64 | 6 |
| ATOM | 14239 | C | ALA | G | 667 | 49.896 | 44.031 | 37.227 | 1.00 | 72.96 | 6 |
| ATOM | 14240 | O | ALA | G | 667 | 50.666 | 43.858 | 38.179 | 1.00 | 73.12 | 8 |
| ATOM | 14241 | N | PRO | G | 668 | 50.076 | 43.419 | 36.049 | 1.00 | 53.70 | 7 |
| ATOM | 14242 | CD | PRO | G | 668 | 49.233 | 43.557 | 34.851 | 1.00 | 164.07 | 6 |
| ATOM | 14243 | CA | PRO | G | 668 | 51.196 | 42.509 | 35.795 | 1.00 | 53.37 | 6 |
| ATOM | 14244 | CB | PRO | G | 668 | 50.948 | 42.051 | 34.356 | 1.00 | 164.48 | 6 |
| ATOM | 14245 | CG | PRO | G | 668 | 50.189 | 43.186 | 33.756 | 1.00 | 164.69 | 6 |
| ATOM | 14246 | C | PRO | G | 668 | 52.599 | 43.067 | 36.002 | 1.00 | 52.84 | 6 |
| ATOM | 14247 | O | PRO | G | 668 | 53.227 | 42.800 | 37.028 | 1.00 | 52.68 | 8 |
| ATOM | 14248 | N | ASN | G | 669 | 53.089 | 43.829 | 35.026 | 1.00 | 128.24 | 7 |
| ATOM | 14249 | CA | ASN | G | 669 | 54.431 | 44.407 | 35.096 | 1.00 | 128.44 | 6 |
| ATOM | 14250 | CB | ASN | G | 669 | 55.278 | 43.910 | 33.916 | 1.00 | 145.71 | 6 |
| ATOM | 14251 | CG | ASN | G | 669 | 56.743 | 44.310 | 34.029 | 1.00 | 146.68 | 6 |
| ATOM | 14252 | OD1 | ASN | G | 669 | 57.072 | 45.493 | 34.119 | 1.00 | 147.81 | 8 |
| ATOM | 14253 | ND2 | ASN | G | 669 | 57.631 | 43.319 | 34.016 | 1.00 | 146.26 | 7 |
| ATOM | 14254 | C | ASN | G | 669 | 54.382 | 45.930 | 35.084 | 1.00 | 127.00 | 6 |
| ATOM | 14255 | O | ASN | G | 669 | 53.428 | 46.522 | 34.583 | 1.00 | 126.52 | 8 |
| ATOM | 14256 | N | VAL | G | 670 | 55.415 | 46.557 | 35.642 | 1.00 | 63.00 | 7 |
| ATOM | 14257 | CA | VAL | G | 670 | 55.484 | 48.011 | 35.693 | 1.00 | 61.29 | 6 |
| ATOM | 14258 | CB | VAL | G | 670 | 56.821 | 48.503 | 36.288 | 1.00 | 53.47 | 6 |
| ATOM | 14259 | CG1 | VAL | G | 670 | 56.793 | 50.022 | 36.432 | 1.00 | 52.98 | 6 |
| ATOM | 14260 | CG2 | VAL | G | 670 | 57.064 | 47.849 | 37.634 | 1.00 | 51.91 | 6 |
| ATOM | 14261 | C | VAL | G | 670 | 55.341 | 48.549 | 34.281 | 1.00 | 60.90 | 6 |
| ATOM | 14262 | O | VAL | G | 670 | 54.912 | 49.682 | 34.071 | 1.00 | 60.85 | 8 |
| ATOM | 14263 | N | LYS | G | 671 | 55.706 | 47.724 | 33.311 | 1.00 | 49.22 | 7 |
| ATOM | 14264 | CA | LYS | G | 671 | 55.591 | 48.119 | 31.919 | 1.00 | 50.52 | 6 |
| ATOM | 14265 | CB | LYS | G | 671 | 56.176 | 47.041 | 31.008 | 1.00 | 104.96 | 6 |
| ATOM | 14266 | CG | LYS | G | 671 | 57.603 | 46.625 | 31.344 | 1.00 | 105.63 | 6 |
| ATOM | 14267 | CD | LYS | G | 671 | 58.609 | 47.718 | 31.037 | 1.00 | 105.64 | 6 |
| ATOM | 14268 | CE | LYS | G | 671 | 60.023 | 47.235 | 31.308 | 1.00 | 105.25 | 6 |
| ATOM | 14269 | NZ | LYS | G | 671 | 61.030 | 48.239 | 30.876 | 1.00 | 106.21 | 7 |
| ATOM | 14270 | C | LYS | G | 671 | 54.096 | 48.216 | 31.690 | 1.00 | 50.79 | 6 |
| ATOM | 14271 | O | LYS | G | 671 | 53.591 | 49.156 | 31.080 | 1.00 | 50.20 | 8 |
| ATOM | 14272 | N | ALA | G | 672 | 53.397 | 47.212 | 32.198 | 1.00 | 91.90 | 7 |
| ATOM | 14273 | CA | ALA | G | 672 | 51.957 | 47.149 | 32.079 | 1.00 | 92.72 | 6 |
| ATOM | 14274 | CB | ALA | G | 672 | 51.462 | 45.838 | 32.651 | 1.00 | 54.28 | 6 |
| ATOM | 14275 | C | ALA | G | 672 | 51.387 | 48.315 | 32.862 | 1.00 | 92.79 | 6 |
| ATOM | 14276 | O | ALA | G | 672 | 50.385 | 48.920 | 32.479 | 1.00 | 93.15 | 8 |
| ATOM | 14277 | N | ALA | G | 673 | 52.055 | 48.629 | 33.963 | 1.00 | 68.21 | 7 |
| ATOM | 14278 | CA | ALA | G | 673 | 51.635 | 49.709 | 34.837 | 1.00 | 68.34 | 6 |
| ATOM | 14279 | CB | ALA | G | 673 | 52.749 | 50.031 | 35.827 | 1.00 | 89.90 | 6 |
| ATOM | 14280 | C | ALA | G | 673 | 51.216 | 50.975 | 34.096 | 1.00 | 67.95 | 6 |
| ATOM | 14281 | O | ALA | G | 673 | 50.031 | 51.302 | 34.032 | 1.00 | 67.40 | 8 |
| ATOM | 14282 | N | ARG | G | 674 | 52.190 | 51.681 | 33.535 | 1.00 | 65.41 | 7 |
| ATOM | 14283 | CA | ARG | G | 674 | 51.908 | 52.922 | 32.835 | 1.00 | 67.03 | 6 |
| ATOM | 14284 | CB | ARG | G | 674 | 53.205 | 53.506 | 32.273 | 1.00 | 103.04 | 6 |
| ATOM | 14285 | CG | ARG | G | 674 | 53.078 | 54.949 | 31.835 | 1.00 | 104.24 | 6 |
| ATOM | 14286 | CD | ARG | G | 674 | 52.793 | 55.058 | 30.353 | 1.00 | 104.40 | 6 |
| ATOM | 14287 | NE | ARG | G | 674 | 52.388 | 56.408 | 29.968 | 1.00 | 105.20 | 7 |
| ATOM | 14288 | CZ | ARG | G | 674 | 51.145 | 56.866 | 30.060 | 1.00 | 105.41 | 6 |
| ATOM | 14289 | NH1 | ARG | G | 674 | 50.181 | 56.082 | 30.523 | 1.00 | 104.98 | 7 |
| ATOM | 14290 | NH2 | ARG | G | 674 | 50.863 | 58.106 | 29.684 | 1.00 | 106.31 | 7 |
| ATOM | 14291 | C | ARG | G | 674 | 50.872 | 52.750 | 31.728 | 1.00 | 67.67 | 6 |
| ATOM | 14292 | O | ARG | G | 674 | 50.029 | 53.625 | 31.509 | 1.00 | 67.06 | 8 |
| ATOM | 14293 | N | ARG | G | 675 | 50.916 | 51.615 | 31.040 | 1.00 | 108.48 | 7 |
| ATOM | 14294 | CA | ARG | G | 675 | 49.966 | 51.376 | 29.965 | 1.00 | 108.75 | 6 |
| ATOM | 14295 | CB | ARG | G | 675 | 50.427 | 50.205 | 29.086 | 1.00 | 127.82 | 6 |
| ATOM | 14296 | CG | ARG | G | 675 | 49.981 | 50.321 | 27.623 | 1.00 | 128.88 | 6 |
| ATOM | 14297 | CD | ARG | G | 675 | 50.760 | 49.391 | 26.692 | 1.00 | 129.54 | 6 |
| ATOM | 14298 | NE | ARG | G | 675 | 52.204 | 49.594 | 26.792 | 1.00 | 130.47 | 7 |
| ATOM | 14299 | CZ | ARG | G | 675 | 53.016 | 48.857 | 27.546 | 1.00 | 130.84 | 6 |
| ATOM | 14300 | NH1 | ARG | G | 675 | 52.527 | 47.855 | 28.266 | 1.00 | 130.82 | 7 |
| ATOM | 14301 | NH2 | ARG | G | 675 | 54.315 | 49.131 | 27.589 | 1.00 | 130.16 | 7 |
| ATOM | 14302 | C | ARG | G | 675 | 48.582 | 51.109 | 30.546 | 1.00 | 107.51 | 6 |
| ATOM | 14303 | O | ARG | G | 675 | 47.585 | 51.116 | 29.832 | 1.00 | 107.12 | 8 |
| ATOM | 14304 | N | MET | G | 676 | 48.521 | 50.880 | 31.851 | 1.00 | 60.35 | 7 |
| ATOM | 14305 | CA | MET | G | 676 | 47.239 | 50.639 | 32.495 | 1.00 | 60.57 | 6 |
| ATOM | 14306 | CB | MET | G | 676 | 47.383 | 49.711 | 33.695 | 1.00 | 72.73 | 6 |
| ATOM | 14307 | CG | MET | G | 676 | 46.073 | 49.476 | 34.430 | 1.00 | 72.58 | 6 |
| ATOM | 14308 | SD | MET | G | 676 | 46.295 | 48.294 | 35.745 | 1.00 | 72.42 | 16 |
| ATOM | 14309 | CE | MET | G | 676 | 46.929 | 46.852 | 34.793 | 1.00 | 72.53 | 6 |
| ATOM | 14310 | C | MET | G | 676 | 46.659 | 51.958 | 32.963 | 1.00 | 60.63 | 6 |
| ATOM | 14311 | O | MET | G | 676 | 45.488 | 52.031 | 33.336 | 1.00 | 60.07 | 8 |
| ATOM | 14312 | N | LEU | G | 677 | 47.501 | 52.987 | 32.981 | 1.00 | 65.74 | 7 |
| ATOM | 14313 | CA | LEU | G | 677 | 47.066 | 54.321 | 33.362 | 1.00 | 66.76 | 6 |
| ATOM | 14314 | CB | LEU | G | 677 | 48.085 | 55.023 | 34.274 | 1.00 | 25.52 | 6 |
| ATOM | 14315 | CG | LEU | G | 677 | 48.594 | 54.395 | 35.584 | 1.00 | 24.47 | 6 |
| ATOM | 14316 | CD1 | LEU | G | 677 | 49.595 | 55.345 | 36.213 | 1.00 | 23.03 | 6 |
| ATOM | 14317 | CD2 | LEU | G | 677 | 47.466 | 54.112 | 36.561 | 1.00 | 24.12 | 6 |
| ATOM | 14318 | C | LEU | G | 677 | 47.027 | 55.013 | 32.014 | 1.00 | 68.67 | 6 |
| ATOM | 14319 | O | LEU | G | 677 | 46.622 | 56.164 | 31.893 | 1.00 | 68.96 | 8 |
| ATOM | 14320 | N | GLU | G | 678 | 47.453 | 54.273 | 30.997 | 1.00 | 168.88 | 7 |
| ATOM | 14321 | CA | GLU | G | 678 | 47.500 | 54.770 | 29.633 | 1.00 | 171.52 | 6 |
| ATOM | 14322 | CB | GLU | G | 678 | 48.436 | 53.893 | 28.805 | 1.00 | 87.26 | 6 |
| ATOM | 14323 | CG | GLU | G | 678 | 48.953 | 54.529 | 27.538 | 1.00 | 86.39 | 6 |
| ATOM | 14324 | CD | GLU | G | 678 | 50.389 | 54.947 | 27.677 | 1.00 | 85.88 | 6 |
| ATOM | 14325 | OE1 | GLU | G | 678 | 51.194 | 54.099 | 28.115 | 1.00 | 85.47 | 8 |
| ATOM | 14326 | OE2 | GLU | G | 678 | 50.716 | 56.109 | 27.348 | 1.00 | 85.93 | 8 |
| ATOM | 14327 | C | GLU | G | 678 | 46.110 | 54.785 | 28.997 | 1.00 | 173.21 | 6 |
| ATOM | 14328 | O | GLU | G | 678 | 45.916 | 55.377 | 27.933 | 1.00 | 173.73 | 8 |
| ATOM | 14329 | N | ARG | G | 679 | 45.149 | 54.126 | 29.641 | 1.00 | 82.26 | 7 |
| ATOM | 14330 | CA | ARG | G | 679 | 43.785 | 54.095 | 29.125 | 1.00 | 83.68 | 6 |
| ATOM | 14331 | CB | ARG | G | 679 | 42.916 | 53.107 | 29.912 | 1.00 | 128.49 | 6 |
| ATOM | 14332 | CG | ARG | G | 679 | 43.183 | 51.636 | 29.611 | 1.00 | 129.03 | 6 |
| ATOM | 14333 | CD | ARG | G | 679 | 42.054 | 50.752 | 30.154 | 1.00 | 129.61 | 6 |
| ATOM | 14334 | NE | ARG | G | 679 | 42.181 | 49.354 | 29.737 | 1.00 | 129.74 | 7 |
| ATOM | 14335 | CZ | ARG | G | 679 | 41.224 | 48.436 | 29.871 | 1.00 | 129.57 | 6 |
| ATOM | 14336 | NH1 | ARG | G | 679 | 40.055 | 48.760 | 30.411 | 1.00 | 129.12 | 7 |
| ATOM | 14337 | NH2 | ARG | G | 679 | 41.435 | 47.191 | 29.462 | 1.00 | 129.43 | 7 |
| ATOM | 14338 | C | ARG | G | 679 | 43.180 | 55.492 | 29.216 | 1.00 | 84.53 | 6 |
| ATOM | 14339 | O | ARG | G | 679 | 41.993 | 55.648 | 29.504 | 1.00 | 84.12 | 8 |
| ATOM | 14340 | N | GLN | G | 680 | 44.025 | 56.491 | 28.970 | 1.00 | 153.00 | 7 |
| ATOM | 14341 | CA | GLN | G | 680 | 43.666 | 57.905 | 28.983 | 1.00 | 154.45 | 6 |
| ATOM | 14342 | CB | GLN | G | 680 | 43.039 | 58.291 | 27.634 | 1.00 | 144.51 | 6 |
| ATOM | 14343 | CG | GLN | G | 680 | 41.873 | 57.409 | 27.184 | 1.00 | 146.04 | 6 |
| ATOM | 14344 | CD | GLN | G | 680 | 41.491 | 57.618 | 25.724 | 1.00 | 147.12 | 6 |
| ATOM | 14345 | OE1 | GLN | G | 680 | 40.546 | 57.003 | 25.225 | 1.00 | 147.19 | 8 |
| ATOM | 14346 | NE2 | GLN | G | 680 | 42.227 | 58.483 | 25.032 | 1.00 | 148.35 | 7 |
| ATOM | 14347 | C | GLN | G | 680 | 42.777 | 58.375 | 30.132 | 1.00 | 154.84 | 6 |
| ATOM | 14348 | O | GLN | G | 680 | 43.148 | 59.289 | 30.871 | 1.00 | 155.73 | 8 |
| ATOM | 14349 | N | ARG | G | 681 | 41.609 | 57.761 | 30.284 | 1.00 | 139.08 | 7 |
| ATOM | 14350 | CA | ARG | G | 681 | 40.684 | 58.144 | 31.341 | 1.00 | 138.36 | 6 |
| ATOM | 14351 | CB | ARG | G | 681 | 39.772 | 59.263 | 30.832 | 1.00 | 91.06 | 6 |
| ATOM | 14352 | CG | ARG | G | 681 | 40.552 | 60.517 | 30.464 | 1.00 | 91.28 | 6 |
| ATOM | 14353 | CD | ARG | G | 681 | 39.747 | 61.508 | 29.641 | 1.00 | 92.01 | 6 |
| ATOM | 14354 | NE | ARG | G | 681 | 40.460 | 62.777 | 29.495 | 1.00 | 92.46 | 7 |
| ATOM | 14355 | CZ | ARG | G | 681 | 39.964 | 63.854 | 28.895 | 1.00 | 92.95 | 6 |
| ATOM | 14356 | NH1 | ARG | G | 681 | 38.745 | 63.828 | 28.371 | 1.00 | 93.70 | 7 |
| ATOM | 14357 | NH2 | ARG | G | 681 | 40.681 | 64.967 | 28.830 | 1.00 | 92.75 | 7 |
| ATOM | 14358 | C | ARG | G | 681 | 39.859 | 56.956 | 31.827 | 1.00 | 137.79 | 6 |
| ATOM | 14359 | O | ARG | G | 681 | 39.253 | 56.244 | 31.026 | 1.00 | 137.92 | 8 |
| ATOM | 14360 | N | ASP | G | 682 | 39.867 | 56.766 | 33.148 | 1.00 | 104.99 | 7 |
| ATOM | 14361 | CA | ASP | G | 682 | 39.160 | 55.698 | 33.864 | 1.00 | 103.46 | 6 |
| ATOM | 14362 | CB | ASP | G | 682 | 38.302 | 54.839 | 32.928 | 1.00 | 163.15 | 6 |
| ATOM | 14363 | CG | ASP | G | 682 | 36.966 | 55.480 | 32.607 | 1.00 | 165.34 | 6 |
| ATOM | 14364 | OD1 | ASP | G | 682 | 36.219 | 55.807 | 33.555 | 1.00 | 165.78 | 8 |
| ATOM | 14365 | OD2 | ASP | G | 682 | 36.663 | 55.654 | 31.407 | 1.00 | 166.58 | 8 |
| ATOM | 14366 | C | ASP | G | 682 | 40.116 | 54.788 | 34.625 | 1.00 | 101.32 | 6 |
| ATOM | 14367 | O | ASP | G | 682 | 40.609 | 53.793 | 34.096 | 1.00 | 101.09 | 8 |
| ATOM | 14368 | N | ILE | G | 683 | 40.374 | 55.144 | 35.875 | 1.00 | 86.94 | 7 |
| ATOM | 14369 | CA | ILE | G | 683 | 41.249 | 54.363 | 36.736 | 1.00 | 83.46 | 6 |
| ATOM | 14370 | CB | ILE | G | 683 | 42.314 | 55.232 | 37.387 | 1.00 | 51.21 | 6 |
| ATOM | 14371 | CG2 | ILE | G | 683 | 43.171 | 54.373 | 38.304 | 1.00 | 50.34 | 6 |
| ATOM | 14372 | CG1 | ILE | G | 683 | 43.127 | 55.959 | 36.324 | 1.00 | 50.15 | 6 |
| ATOM | 14373 | CD1 | ILE | G | 683 | 44.025 | 57.015 | 36.916 | 1.00 | 50.36 | 6 |
| ATOM | 14374 | C | ILE | G | 683 | 40.377 | 53.841 | 37.860 | 1.00 | 81.93 | 6 |
| ATOM | 14375 | O | ILE | G | 683 | 39.948 | 54.615 | 38.712 | 1.00 | 81.42 | 8 |
| ATOM | 14376 | N | LYS | G | 684 | 40.106 | 52.545 | 37.889 | 1.00 | 76.45 | 7 |
| ATOM | 14377 | CA | LYS | G | 684 | 39.257 | 52.065 | 38.962 | 1.00 | 74.88 | 6 |
| ATOM | 14378 | CB | LYS | G | 684 | 38.789 | 50.624 | 38.743 | 1.00 | 122.35 | 6 |
| ATOM | 14379 | CG | LYS | G | 684 | 37.513 | 50.306 | 39.537 | 1.00 | 123.25 | 6 |
| ATOM | 14380 | CD | LYS | G | 684 | 37.482 | 48.874 | 40.054 | 1.00 | 124.08 | 6 |
| ATOM | 14381 | CE | LYS | G | 684 | 36.303 | 48.657 | 40.998 | 1.00 | 124.43 | 6 |
| ATOM | 14382 | NZ | LYS | G | 684 | 36.359 | 47.338 | 41.692 | 1.00 | 124.41 | 7 |
| ATOM | 14383 | C | LYS | G | 684 | 39.992 | 52.160 | 40.279 | 1.00 | 72.87 | 6 |
| ATOM | 14384 | O | LYS | G | 684 | 41.218 | 52.069 | 40.344 | 1.00 | 72.45 | 8 |
| ATOM | 14385 | N | ASP | G | 685 | 39.204 | 52.350 | 41.324 | 1.00 | 46.96 | 7 |
| ATOM | 14386 | CA | ASP | G | 685 | 39.688 | 52.480 | 42.685 | 1.00 | 44.48 | 6 |
| ATOM | 14387 | CB | ASP | G | 685 | 38.530 | 52.193 | 43.654 | 1.00 | 38.62 | 6 |
| ATOM | 14388 | CG | ASP | G | 685 | 37.279 | 51.678 | 42.946 | 1.00 | 36.98 | 6 |
| ATOM | 14389 | OD1 | ASP | G | 685 | 36.782 | 52.331 | 42.000 | 1.00 | 35.77 | 8 |
| ATOM | 14390 | OD2 | ASP | G | 685 | 36.790 | 50.613 | 43.354 | 1.00 | 37.11 | 8 |
| ATOM | 14391 | C | ASP | G | 685 | 40.911 | 51.644 | 43.073 | 1.00 | 43.47 | 6 |
| ATOM | 14392 | O | ASP | G | 685 | 41.707 | 52.070 | 43.902 | 1.00 | 42.39 | 8 |
| ATOM | 14393 | N | GLU | G | 686 | 41.070 | 50.465 | 42.481 | 1.00 | 69.13 | 7 |
| ATOM | 14394 | CA | GLU | G | 686 | 42.200 | 49.600 | 42.819 | 1.00 | 68.84 | 6 |
| ATOM | 14395 | CB | GLU | G | 686 | 42.256 | 48.417 | 41.858 | 1.00 | 127.87 | 6 |
| ATOM | 14396 | CG | GLU | G | 686 | 40.977 | 47.618 | 41.806 | 1.00 | 130.65 | 6 |
| ATOM | 14397 | CD | GLU | G | 686 | 41.166 | 46.299 | 41.100 | 1.00 | 132.99 | 6 |
| ATOM | 14398 | OE1 | GLU | G | 686 | 41.989 | 45.491 | 41.583 | 1.00 | 135.19 | 8 |
| ATOM | 14399 | OE2 | GLU | G | 686 | 40.501 | 46.071 | 40.068 | 1.00 | 132.61 | 8 |
| ATOM | 14400 | C | GLU | G | 686 | 43.545 | 50.329 | 42.812 | 1.00 | 66.66 | 6 |
| ATOM | 14401 | O | GLU | G | 686 | 44.486 | 49.929 | 43.505 | 1.00 | 66.14 | 8 |
| ATOM | 14402 | N | VAL | G | 687 | 43.632 | 51.392 | 42.016 | 1.00 | 69.55 | 7 |
| ATOM | 14403 | CA | VAL | G | 687 | 44.851 | 52.174 | 41.934 | 1.00 | 66.75 | 6 |
| ATOM | 14404 | CB | VAL | G | 687 | 45.239 | 52.471 | 40.484 | 1.00 | 79.16 | 6 |
| ATOM | 14405 | CG1 | VAL | G | 687 | 46.303 | 53.553 | 40.435 | 1.00 | 78.97 | 6 |
| ATOM | 14406 | CG2 | VAL | G | 687 | 45.789 | 51.216 | 39.851 | 1.00 | 79.57 | 6 |
| ATOM | 14407 | C | VAL | G | 687 | 44.718 | 53.472 | 42.707 | 1.00 | 64.40 | 6 |
| ATOM | 14408 | O | VAL | G | 687 | 45.658 | 53.872 | 43.379 | 1.00 | 64.19 | 8 |
| ATOM | 14409 | N | TRP | G | 688 | 43.572 | 54.144 | 42.624 | 1.00 | 34.82 | 7 |
| ATOM | 14410 | CA | TRP | G | 688 | 43.430 | 55.369 | 43.400 | 1.00 | 32.17 | 6 |
| ATOM | 14411 | CB | TRP | G | 688 | 41.989 | 55.844 | 43.443 | 1.00 | 42.38 | 6 |
| ATOM | 14412 | CG | TRP | G | 688 | 41.674 | 56.816 | 42.383 | 1.00 | 40.93 | 6 |
| ATOM | 14413 | CD2 | TRP | G | 688 | 42.522 | 57.840 | 41.883 | 1.00 | 39.77 | 6 |
| ATOM | 14414 | CE2 | TRP | G | 688 | 41.824 | 58.495 | 40.852 | 1.00 | 39.82 | 6 |
| ATOM | 14415 | CE3 | TRP | G | 688 | 43.806 | 58.267 | 42.197 | 1.00 | 40.07 | 6 |
| ATOM | 14416 | CD1 | TRP | G | 688 | 40.526 | 56.892 | 41.667 | 1.00 | 41.78 | 6 |
| ATOM | 14417 | NE1 | TRP | G | 688 | 40.601 | 57.895 | 40.741 | 1.00 | 41.05 | 7 |
| ATOM | 14418 | CZ2 | TRP | G | 688 | 42.362 | 59.548 | 40.138 | 1.00 | 39.17 | 6 |
| ATOM | 14419 | CZ3 | TRP | G | 688 | 44.343 | 59.315 | 41.486 | 1.00 | 40.39 | 6 |
| ATOM | 14420 | CH2 | TRP | G | 688 | 43.622 | 59.943 | 40.466 | 1.00 | 40.16 | 6 |
| ATOM | 14421 | C | TRP | G | 688 | 43.902 | 55.069 | 44.811 | 1.00 | 31.35 | 6 |
| ATOM | 14422 | O | TRP | G | 688 | 44.760 | 55.767 | 45.347 | 1.00 | 31.58 | 8 |
| ATOM | 14423 | N | ASP | G | 689 | 43.357 | 54.011 | 45.398 | 1.00 | 36.23 | 7 |
| ATOM | 14424 | CA | ASP | G | 689 | 43.735 | 53.606 | 46.741 | 1.00 | 37.19 | 6 |
| ATOM | 14425 | CB | ASP | G | 689 | 43.048 | 52.289 | 47.130 | 1.00 | 84.86 | 6 |
| ATOM | 14426 | CG | ASP | G | 689 | 41.618 | 52.484 | 47.602 | 1.00 | 87.86 | 6 |
| ATOM | 14427 | OD1 | ASP | G | 689 | 41.361 | 53.479 | 48.317 | 1.00 | 89.18 | 8 |
| ATOM | 14428 | OD2 | ASP | G | 689 | 40.760 | 51.631 | 47.274 | 1.00 | 88.73 | 8 |
| ATOM | 14429 | C | ASP | G | 689 | 45.248 | 53.429 | 46.863 | 1.00 | 36.89 | 6 |
| ATOM | 14430 | O | ASP | G | 689 | 45.731 | 52.814 | 47.826 | 1.00 | 37.89 | 8 |
| ATOM | 14431 | N | ALA | G | 690 | 45.997 | 53.957 | 45.896 | 1.00 | 36.69 | 7 |
| ATOM | 14432 | CA | ALA | G | 690 | 47.452 | 53.859 | 45.925 | 1.00 | 35.82 | 6 |
| ATOM | 14433 | CB | ALA | G | 690 | 47.981 | 53.525 | 44.549 | 1.00 | 88.29 | 6 |
| ATOM | 14434 | C | ALA | G | 690 | 48.115 | 55.132 | 46.444 | 1.00 | 35.02 | 6 |
| ATOM | 14435 | O | ALA | G | 690 | 48.864 | 55.093 | 47.419 | 1.00 | 33.34 | 8 |
| ATOM | 14436 | N | LEU | G | 691 | 47.857 | 56.256 | 45.791 | 1.00 | 49.11 | 7 |
| ATOM | 14437 | CA | LEU | G | 691 | 48.454 | 57.496 | 46.245 | 1.00 | 49.90 | 6 |
| ATOM | 14438 | CB | LEU | G | 691 | 48.005 | 58.671 | 45.373 | 1.00 | 13.87 | 6 |
| ATOM | 14439 | CG | LEU | G | 691 | 48.000 | 58.476 | 43.864 | 1.00 | 13.87 | 6 |
| ATOM | 14440 | CD1 | LEU | G | 691 | 46.955 | 57.450 | 43.541 | 1.00 | 13.87 | 6 |
| ATOM | 14441 | CD2 | LEU | G | 691 | 47.696 | 59.765 | 43.155 | 1.00 | 13.87 | 6 |
| ATOM | 14442 | C | LEU | G | 691 | 47.959 | 57.681 | 47.679 | 1.00 | 52.48 | 6 |
| ATOM | 14443 | O | LEU | G | 691 | 48.702 | 58.115 | 48.566 | 1.00 | 53.88 | 8 |
| ATOM | 14444 | N | GLU | G | 692 | 46.695 | 57.335 | 47.908 | 1.00 | 97.21 | 7 |
| ATOM | 14445 | CA | GLU | G | 692 | 46.127 | 57.458 | 49.241 | 1.00 | 98.91 | 6 |
| ATOM | 14446 | CB | GLU | G | 692 | 44.691 | 56.904 | 49.282 | 1.00 | 73.29 | 6 |
| ATOM | 14447 | CG | GLU | G | 692 | 43.657 | 57.771 | 48.529 | 1.00 | 74.54 | 6 |
| ATOM | 14448 | CD | GLU | G | 692 | 42.246 | 57.166 | 48.487 | 1.00 | 74.13 | 6 |
| ATOM | 14449 | OE1 | GLU | G | 692 | 41.698 | 56.858 | 49.564 | 1.00 | 74.43 | 8 |
| ATOM | 14450 | OE2 | GLU | G | 692 | 41.678 | 57.009 | 47.381 | 1.00 | 73.17 | 8 |
| ATOM | 14451 | C | GLU | G | 692 | 47.045 | 56.672 | 50.161 | 1.00 | 99.71 | 6 |
| ATOM | 14452 | O | GLU | G | 692 | 46.965 | 56.794 | 51.383 | 1.00 | 101.25 | 8 |
| ATOM | 14453 | N | GLU | G | 693 | 47.925 | 55.872 | 49.556 | 1.00 | 33.70 | 7 |
| ATOM | 14454 | CA | GLU | G | 693 | 48.896 | 55.082 | 50.304 | 1.00 | 34.46 | 6 |
| ATOM | 14455 | CB | GLU | G | 693 | 48.758 | 53.589 | 50.003 | 1.00 | 74.88 | 6 |
| ATOM | 14456 | CG | GLU | G | 693 | 49.753 | 52.758 | 50.810 | 1.00 | 76.85 | 6 |
| ATOM | 14457 | CD | GLU | G | 693 | 49.538 | 51.266 | 50.693 | 1.00 | 78.68 | 6 |
| ATOM | 14458 | OE1 | GLU | G | 693 | 48.424 | 50.798 | 51.016 | 1.00 | 80.74 | 8 |
| ATOM | 14459 | OE2 | GLU | G | 693 | 50.489 | 50.562 | 50.288 | 1.00 | 78.64 | 8 |
| ATOM | 14460 | C | GLU | G | 693 | 50.340 | 55.516 | 50.035 | 1.00 | 34.30 | 6 |
| ATOM | 14461 | O | GLU | G | 693 | 51.218 | 55.305 | 50.872 | 1.00 | 34.92 | 8 |
| ATOM | 14462 | N | VAL | G | 694 | 50.592 | 56.111 | 48.869 | 1.00 | 62.59 | 7 |
| ATOM | 14463 | CA | VAL | G | 694 | 51.937 | 56.599 | 48.520 | 1.00 | 61.55 | 6 |
| ATOM | 14464 | CB | VAL | G | 694 | 52.059 | 56.888 | 47.012 | 1.00 | 42.74 | 6 |
| ATOM | 14465 | CG1 | VAL | G | 694 | 53.492 | 57.256 | 46.661 | 1.00 | 42.49 | 6 |
| ATOM | 14466 | CG2 | VAL | G | 694 | 51.591 | 55.692 | 46.227 | 1.00 | 43.31 | 6 |
| ATOM | 14467 | C | VAL | G | 694 | 52.120 | 57.919 | 49.257 | 1.00 | 60.03 | 6 |
| ATOM | 14468 | O | VAL | G | 694 | 52.919 | 58.033 | 50.190 | 1.00 | 58.95 | 8 |
| ATOM | 14469 | N | ILE | G | 695 | 51.356 | 58.905 | 48.802 | 1.00 | 46.85 | 7 |
| ATOM | 14470 | CA | ILE | G | 695 | 51.328 | 60.234 | 49.374 | 1.00 | 45.93 | 6 |
| ATOM | 14471 | CB | ILE | G | 695 | 49.973 | 60.907 | 49.079 | 1.00 | 45.86 | 6 |
| ATOM | 14472 | CG2 | ILE | G | 695 | 49.745 | 62.097 | 50.004 | 1.00 | 45.84 | 6 |
| ATOM | 14473 | CG1 | ILE | G | 695 | 49.912 | 61.321 | 47.619 | 1.00 | 46.05 | 6 |
| ATOM | 14474 | CD1 | ILE | G | 695 | 48.638 | 62.051 | 47.274 | 1.00 | 46.62 | 6 |
| ATOM | 14475 | C | ILE | G | 695 | 51.475 | 60.143 | 50.882 | 1.00 | 45.93 | 6 |
| ATOM | 14476 | O | ILE | G | 695 | 51.982 | 61.060 | 51.530 | 1.00 | 46.59 | 8 |
| ATOM | 14477 | N | HIS | G | 696 | 51.029 | 59.027 | 51.440 | 1.00 | 37.20 | 7 |
| ATOM | 14478 | CA | HIS | G | 696 | 51.077 | 58.857 | 52.877 | 1.00 | 37.10 | 6 |
| ATOM | 14479 | CB | HIS | G | 696 | 50.621 | 57.466 | 53.275 | 1.00 | 62.35 | 6 |
| ATOM | 14480 | CG | HIS | G | 696 | 49.890 | 57.446 | 54.573 | 1.00 | 65.83 | 6 |
| ATOM | 14481 | CD2 | HIS | G | 696 | 50.312 | 57.169 | 55.827 | 1.00 | 67.81 | 6 |
| ATOM | 14482 | ND1 | HIS | G | 696 | 48.573 | 57.835 | 54.685 | 1.00 | 67.75 | 7 |
| ATOM | 14483 | CE1 | HIS | G | 696 | 48.214 | 57.798 | 55.955 | 1.00 | 68.94 | 6 |
| ATOM | 14484 | NE2 | HIS | G | 696 | 49.251 | 57.399 | 56.669 | 1.00 | 68.75 | 7 |
| ATOM | 14485 | C | HIS | G | 696 | 52.432 | 59.126 | 53.486 | 1.00 | 34.92 | 6 |
| ATOM | 14486 | O | HIS | G | 696 | 53.402 | 58.414 | 53.222 | 1.00 | 34.04 | 8 |
| ATOM | 14487 | N | GLY | G | 697 | 52.479 | 60.166 | 54.313 | 1.00 | 61.97 | 7 |
| ATOM | 14488 | CA | GLY | G | 697 | 53.711 | 60.540 | 54.976 | 1.00 | 60.50 | 6 |
| ATOM | 14489 | C | GLY | G | 697 | 54.517 | 61.512 | 54.149 | 1.00 | 59.15 | 6 |
| ATOM | 14490 | O | GLY | G | 697 | 55.432 | 62.161 | 54.644 | 1.00 | 59.68 | 8 |
| ATOM | 14491 | N | LYS | G | 698 | 54.176 | 61.615 | 52.875 | 1.00 | 32.49 | 7 |
| ATOM | 14492 | CA | LYS | G | 698 | 54.894 | 62.505 | 51.997 | 1.00 | 30.03 | 6 |
| ATOM | 14493 | CB | LYS | G | 698 | 54.908 | 61.952 | 50.574 | 1.00 | 59.08 | 6 |
| ATOM | 14494 | CG | LYS | G | 698 | 55.761 | 60.705 | 50.420 | 1.00 | 61.54 | 6 |
| ATOM | 14495 | CD | LYS | G | 698 | 55.662 | 60.129 | 49.031 | 1.00 | 63.05 | 6 |
| ATOM | 14496 | CE | LYS | G | 698 | 56.521 | 58.890 | 48.915 | 1.00 | 64.81 | 6 |
| ATOM | 14497 | NZ | LYS | G | 698 | 56.228 | 58.133 | 47.662 | 1.00 | 66.75 | 7 |
| ATOM | 14498 | C | LYS | G | 698 | 54.250 | 63.853 | 52.015 | 1.00 | 27.60 | 6 |
| ATOM | 14499 | O | LYS | G | 698 | 53.036 | 63.973 | 52.102 | 1.00 | 27.52 | 8 |
| ATOM | 14500 | N | VAL | G | 699 | 55.083 | 64.874 | 51.951 | 1.00 | 33.63 | 7 |
| ATOM | 14501 | CA | VAL | G | 699 | 54.604 | 66.230 | 51.938 | 1.00 | 32.05 | 6 |
| ATOM | 14502 | CB | VAL | G | 699 | 55.314 | 67.082 | 52.960 | 1.00 | 18.04 | 6 |
| ATOM | 14503 | CG1 | VAL | G | 699 | 55.126 | 66.500 | 54.326 | 1.00 | 18.53 | 6 |
| ATOM | 14504 | CG2 | VAL | G | 699 | 56.773 | 67.201 | 52.596 | 1.00 | 19.26 | 6 |
| ATOM | 14505 | C | VAL | G | 699 | 54.944 | 66.769 | 50.576 | 1.00 | 30.37 | 6 |
| ATOM | 14506 | O | VAL | G | 699 | 55.728 | 66.163 | 49.851 | 1.00 | 30.18 | 8 |
| ATOM | 14507 | N | VAL | G | 700 | 54.362 | 67.910 | 50.237 | 1.00 | 47.17 | 7 |
| ATOM | 14508 | CA | VAL | G | 700 | 54.596 | 68.544 | 48.952 | 1.00 | 44.94 | 6 |
| ATOM | 14509 | CB | VAL | G | 700 | 53.392 | 68.344 | 48.028 | 1.00 | 13.87 | 6 |
| ATOM | 14510 | CG1 | VAL | G | 700 | 53.590 | 69.095 | 46.740 | 1.00 | 13.87 | 6 |
| ATOM | 14511 | CG2 | VAL | G | 700 | 53.226 | 66.882 | 47.730 | 1.00 | 13.87 | 6 |
| ATOM | 14512 | C | VAL | G | 700 | 54.809 | 70.032 | 49.173 | 1.00 | 45.13 | 6 |
| ATOM | 14513 | O | VAL | G | 700 | 54.151 | 70.623 | 50.025 | 1.00 | 46.25 | 8 |
| ATOM | 14514 | N | LEU | G | 701 | 55.733 | 70.635 | 48.425 | 1.00 | 31.56 | 7 |
| ATOM | 14515 | CA | LEU | G | 701 | 56.006 | 72.065 | 48.549 | 1.00 | 30.51 | 6 |
| ATOM | 14516 | CB | LEU | G | 701 | 57.398 | 72.389 | 48.014 | 1.00 | 19.40 | 6 |
| ATOM | 14517 | CG | LEU | G | 701 | 58.572 | 72.159 | 48.967 | 1.00 | 19.97 | 6 |
| ATOM | 14518 | CD1 | LEU | G | 701 | 58.323 | 70.961 | 49.838 | 1.00 | 21.06 | 6 |
| ATOM | 14519 | CD2 | LEU | G | 701 | 59.839 | 71.957 | 48.181 | 1.00 | 20.10 | 6 |
| ATOM | 14520 | C | LEU | G | 701 | 54.968 | 72.828 | 47.758 | 1.00 | 31.25 | 6 |
| ATOM | 14521 | O | LEU | G | 701 | 54.595 | 72.417 | 46.662 | 1.00 | 31.89 | 8 |
| ATOM | 14522 | N | LEU | G | 702 | 54.484 | 73.928 | 48.318 | 1.00 | 13.87 | 7 |
| ATOM | 14523 | CA | LEU | G | 702 | 53.486 | 74.743 | 47.625 | 1.00 | 13.87 | 6 |
| ATOM | 14524 | CB | LEU | G | 702 | 52.242 | 74.881 | 48.513 | 1.00 | 13.87 | 6 |
| ATOM | 14525 | CG | LEU | G | 702 | 50.864 | 74.734 | 47.879 | 1.00 | 13.87 | 6 |
| ATOM | 14526 | CD1 | LEU | G | 702 | 50.695 | 75.788 | 46.818 | 1.00 | 13.87 | 6 |
| ATOM | 14527 | CD2 | LEU | G | 702 | 50.730 | 73.382 | 47.268 | 1.00 | 13.87 | 6 |
| ATOM | 14528 | C | LEU | G | 702 | 54.159 | 76.101 | 47.362 | 1.00 | 16.31 | 6 |
| ATOM | 14529 | O | LEU | G | 702 | 54.962 | 76.538 | 48.171 | 1.00 | 15.48 | 8 |
| ATOM | 14530 | N | ASN | G | 703 | 53.880 | 76.766 | 46.245 | 1.00 | 34.74 | 7 |
| ATOM | 14531 | CA | ASN | G | 703 | 54.563 | 78.039 | 46.002 | 1.00 | 39.25 | 6 |
| ATOM | 14532 | CB | ASN | G | 703 | 55.876 | 77.807 | 45.251 | 1.00 | 63.81 | 6 |
| ATOM | 14533 | CG | ASN | G | 703 | 56.366 | 79.059 | 44.529 | 1.00 | 65.71 | 6 |
| ATOM | 14534 | OD1 | ASN | G | 703 | 56.243 | 80.170 | 45.044 | 1.00 | 69.41 | 8 |
| ATOM | 14535 | ND2 | ASN | G | 703 | 56.928 | 78.881 | 43.335 | 1.00 | 64.14 | 7 |
| ATOM | 14536 | C | ASN | G | 703 | 53.803 | 79.120 | 45.275 | 1.00 | 40.59 | 6 |
| ATOM | 14537 | O | ASN | G | 703 | 53.155 | 78.860 | 44.268 | 1.00 | 42.16 | 8 |
| ATOM | 14538 | N | ARG | G | 704 | 53.940 | 80.345 | 45.771 | 1.00 | 50.15 | 7 |
| ATOM | 14539 | CA | ARG | G | 704 | 53.276 | 81.509 | 45.195 | 1.00 | 51.77 | 6 |
| ATOM | 14540 | CB | ARG | G | 704 | 52.660 | 82.354 | 46.305 | 1.00 | 71.61 | 6 |
| ATOM | 14541 | CG | ARG | G | 704 | 52.414 | 83.815 | 45.942 | 1.00 | 72.56 | 6 |
| ATOM | 14542 | CD | ARG | G | 704 | 51.095 | 84.021 | 45.251 | 1.00 | 71.72 | 6 |
| ATOM | 14543 | NE | ARG | G | 704 | 50.763 | 85.437 | 45.220 | 1.00 | 71.33 | 7 |
| ATOM | 14544 | CZ | ARG | G | 704 | 49.553 | 85.901 | 44.945 | 1.00 | 72.09 | 6 |
| ATOM | 14545 | NH1 | ARG | G | 704 | 48.577 | 85.053 | 44.679 | 1.00 | 73.18 | 7 |
| ATOM | 14546 | NH2 | ARG | G | 704 | 49.313 | 87.203 | 44.948 | 1.00 | 72.05 | 7 |
| ATOM | 14547 | C | ARG | G | 704 | 54.199 | 82.390 | 44.361 | 1.00 | 52.25 | 6 |
| ATOM | 14548 | O | ARG | G | 704 | 55.392 | 82.505 | 44.647 | 1.00 | 53.60 | 8 |
| ATOM | 14549 | N | ALA | G | 705 | 53.597 | 83.032 | 43.359 | 1.00 | 59.29 | 7 |
| ATOM | 14550 | CA | ALA | G | 705 | 54.249 | 83.922 | 42.399 | 1.00 | 58.39 | 6 |
| ATOM | 14551 | CB | ALA | G | 705 | 53.238 | 84.941 | 41.897 | 1.00 | 32.08 | 6 |
| ATOM | 14552 | C | ALA | G | 705 | 55.545 | 84.625 | 42.811 | 1.00 | 58.27 | 6 |
| ATOM | 14553 | O | ALA | G | 705 | 56.603 | 83.996 | 42.855 | 1.00 | 60.43 | 8 |
| ATOM | 14554 | N | PRO | G | 706 | 55.498 | 85.935 | 43.107 | 1.00 | 47.84 | 7 |
| ATOM | 14555 | CD | PRO | G | 706 | 54.396 | 86.843 | 43.468 | 1.00 | 17.08 | 6 |
| ATOM | 14556 | CA | PRO | G | 706 | 56.779 | 86.531 | 43.484 | 1.00 | 47.16 | 6 |
| ATOM | 14557 | CB | PRO | G | 706 | 56.451 | 88.003 | 43.527 | 1.00 | 17.01 | 6 |
| ATOM | 14558 | CG | PRO | G | 706 | 55.126 | 87.975 | 44.165 | 1.00 | 16.10 | 6 |
| ATOM | 14559 | C | PRO | G | 706 | 57.174 | 86.004 | 44.846 | 1.00 | 47.25 | 6 |
| ATOM | 14560 | O | PRO | G | 706 | 56.429 | 86.168 | 45.807 | 1.00 | 47.58 | 8 |
| ATOM | 14561 | N | THR | G | 707 | 58.334 | 85.372 | 44.944 | 1.00 | 58.75 | 7 |
| ATOM | 14562 | CA | THR | G | 707 | 58.743 | 84.833 | 46.228 | 1.00 | 57.99 | 6 |
| ATOM | 14563 | CB | THR | G | 707 | 59.451 | 83.505 | 46.064 | 1.00 | 13.87 | 6 |
| ATOM | 14564 | OG1 | THR | G | 707 | 58.738 | 82.713 | 45.112 | 1.00 | 13.87 | 8 |
| ATOM | 14565 | CG2 | THR | G | 707 | 59.495 | 82.771 | 47.386 | 1.00 | 13.87 | 6 |
| ATOM | 14566 | C | THR | G | 707 | 59.654 | 85.778 | 46.977 | 1.00 | 58.21 | 6 |
| ATOM | 14567 | O | THR | G | 707 | 60.609 | 86.308 | 46.428 | 1.00 | 60.89 | 8 |
| ATOM | 14568 | N | LEU | G | 708 | 59.342 | 85.980 | 48.244 | 1.00 | 30.92 | 7 |
| ATOM | 14569 | CA | LEU | G | 708 | 60.101 | 86.854 | 49.113 | 1.00 | 30.27 | 6 |
| ATOM | 14570 | CB | LEU | G | 708 | 59.351 | 88.162 | 49.245 | 1.00 | 50.27 | 6 |
| ATOM | 14571 | CG | LEU | G | 708 | 58.937 | 88.593 | 47.835 | 1.00 | 52.01 | 6 |
| ATOM | 14572 | CD1 | LEU | G | 708 | 57.711 | 89.509 | 47.857 | 1.00 | 51.20 | 6 |
| ATOM | 14573 | CD2 | LEU | G | 708 | 60.148 | 89.245 | 47.172 | 1.00 | 52.56 | 6 |
| ATOM | 14574 | C | LEU | G | 708 | 60.078 | 86.063 | 50.398 | 1.00 | 29.61 | 6 |
| ATOM | 14575 | O | LEU | G | 708 | 59.076 | 85.442 | 50.704 | 1.00 | 31.01 | 8 |
| ATOM | 14576 | N | HIS | G | 709 | 61.163 | 86.074 | 51.152 | 1.00 | 20.02 | 7 |
| ATOM | 14577 | CA | HIS | G | 709 | 61.272 | 85.280 | 52.384 | 1.00 | 20.12 | 6 |
| ATOM | 14578 | CB | HIS | G | 709 | 60.578 | 85.957 | 53.596 | 1.00 | 26.13 | 6 |
| ATOM | 14579 | CG | HIS | G | 709 | 59.091 | 86.111 | 53.470 | 1.00 | 24.12 | 6 |
| ATOM | 14580 | CD2 | HIS | G | 709 | 58.075 | 85.401 | 54.012 | 1.00 | 23.49 | 6 |
| ATOM | 14581 | ND1 | HIS | G | 709 | 58.501 | 87.113 | 52.732 | 1.00 | 23.70 | 7 |
| ATOM | 14582 | CE1 | HIS | G | 709 | 57.188 | 87.011 | 52.822 | 1.00 | 22.98 | 6 |
| ATOM | 14583 | NE2 | HIS | G | 709 | 56.903 | 85.981 | 53.593 | 1.00 | 23.50 | 7 |
| ATOM | 14584 | C | HIS | G | 709 | 60.826 | 83.804 | 52.274 | 1.00 | 20.07 | 6 |
| ATOM | 14585 | O | HIS | G | 709 | 59.842 | 83.451 | 51.620 | 1.00 | 18.92 | 8 |
| ATOM | 14586 | N | ARG | G | 710 | 61.605 | 82.943 | 52.919 | 1.00 | 30.46 | 7 |
| ATOM | 14587 | CA | ARG | G | 710 | 61.350 | 81.509 | 52.947 | 1.00 | 29.87 | 6 |
| ATOM | 14588 | CB | ARG | G | 710 | 61.948 | 80.899 | 54.232 | 1.00 | 35.37 | 6 |
| ATOM | 14589 | CG | ARG | G | 710 | 61.425 | 81.525 | 55.516 | 1.00 | 36.40 | 6 |
| ATOM | 14590 | CD | ARG | G | 710 | 61.844 | 80.760 | 56.744 | 1.00 | 36.25 | 6 |
| ATOM | 14591 | NE | ARG | G | 710 | 63.271 | 80.851 | 56.985 | 1.00 | 38.39 | 7 |
| ATOM | 14592 | CZ | ARG | G | 710 | 63.846 | 80.480 | 58.123 | 1.00 | 39.64 | 6 |
| ATOM | 14593 | NH1 | ARG | G | 710 | 63.105 | 79.993 | 59.111 | 1.00 | 38.83 | 7 |
| ATOM | 14594 | NH2 | ARG | G | 710 | 65.155 | 80.613 | 58.286 | 1.00 | 40.25 | 7 |
| ATOM | 14595 | C | ARG | G | 710 | 59.869 | 81.174 | 52.882 | 1.00 | 29.14 | 6 |
| ATOM | 14596 | O | ARG | G | 710 | 59.472 | 80.272 | 52.157 | 1.00 | 29.02 | 8 |
| ATOM | 14597 | N | LEU | G | 711 | 59.058 | 81.904 | 53.647 | 1.00 | 43.22 | 7 |
| ATOM | 14598 | CA | LEU | G | 711 | 57.625 | 81.655 | 53.691 | 1.00 | 43.96 | 6 |
| ATOM | 14599 | CB | LEU | G | 711 | 56.963 | 82.450 | 54.829 | 1.00 | 16.37 | 6 |
| ATOM | 14600 | CG | LEU | G | 711 | 57.333 | 82.109 | 56.279 | 1.00 | 14.11 | 6 |
| ATOM | 14601 | CD1 | LEU | G | 711 | 56.143 | 82.399 | 57.189 | 1.00 | 13.87 | 6 |
| ATOM | 14602 | CD2 | LEU | G | 711 | 57.724 | 80.654 | 56.402 | 1.00 | 13.87 | 6 |
| ATOM | 14603 | C | LEU | G | 711 | 56.891 | 81.908 | 52.377 | 1.00 | 45.04 | 6 |
| ATOM | 14604 | O | LEU | G | 711 | 55.711 | 82.243 | 52.366 | 1.00 | 44.80 | 8 |
| ATOM | 14605 | N | GLY | G | 712 | 57.596 | 81.745 | 51.268 | 1.00 | 40.20 | 7 |
| ATOM | 14606 | CA | GLY | G | 712 | 56.967 | 81.915 | 49.975 | 1.00 | 42.22 | 6 |
| ATOM | 14607 | C | GLY | G | 712 | 56.672 | 80.521 | 49.457 | 1.00 | 42.67 | 6 |
| ATOM | 14608 | O | GLY | G | 712 | 55.797 | 80.307 | 48.613 | 1.00 | 43.12 | 8 |
| ATOM | 14609 | N | ILE | G | 713 | 57.421 | 79.559 | 49.982 | 1.00 | 50.14 | 7 |
| ATOM | 14610 | CA | ILE | G | 713 | 57.247 | 78.175 | 49.600 | 1.00 | 50.51 | 6 |
| ATOM | 14611 | CB | ILE | G | 713 | 58.585 | 77.466 | 49.474 | 1.00 | 52.75 | 6 |
| ATOM | 14612 | CG2 | ILE | G | 713 | 58.367 | 75.975 | 49.301 | 1.00 | 54.09 | 6 |
| ATOM | 14613 | CG1 | ILE | G | 713 | 59.340 | 78.033 | 48.273 | 1.00 | 54.70 | 6 |
| ATOM | 14614 | CD1 | ILE | G | 713 | 60.627 | 77.306 | 47.966 | 1.00 | 55.45 | 6 |
| ATOM | 14615 | C | ILE | G | 713 | 56.367 | 77.416 | 50.584 | 1.00 | 49.30 | 6 |
| ATOM | 14616 | O | ILE | G | 713 | 55.180 | 77.240 | 50.329 | 1.00 | 50.35 | 8 |
| ATOM | 14617 | N | ALA | G | 714 | 56.923 | 76.969 | 51.707 | 1.00 | 30.45 | 7 |
| ATOM | 14618 | CA | ALA | G | 714 | 56.121 | 76.216 | 52.685 | 1.00 | 27.60 | 6 |
| ATOM | 14619 | CB | ALA | G | 714 | 54.934 | 77.079 | 53.186 | 1.00 | 31.44 | 6 |
| ATOM | 14620 | C | ALA | G | 714 | 55.624 | 74.860 | 52.124 | 1.00 | 25.04 | 6 |
| ATOM | 14621 | O | ALA | G | 714 | 54.949 | 74.776 | 51.094 | 1.00 | 23.08 | 8 |
| ATOM | 14622 | N | ALA | G | 715 | 55.984 | 73.792 | 52.814 | 1.00 | 27.33 | 7 |
| ATOM | 14623 | CA | ALA | G | 715 | 55.615 | 72.463 | 52.370 | 1.00 | 27.59 | 6 |
| ATOM | 14624 | CB | ALA | G | 715 | 56.794 | 71.514 | 52.516 | 1.00 | 132.96 | 6 |
| ATOM | 14625 | C | ALA | G | 715 | 54.449 | 71.944 | 53.153 | 1.00 | 26.48 | 6 |
| ATOM | 14626 | O | ALA | G | 715 | 54.288 | 72.269 | 54.326 | 1.00 | 26.56 | 8 |
| ATOM | 14627 | N | PHE | G | 716 | 53.655 | 71.114 | 52.494 | 1.00 | 19.00 | 7 |
| ATOM | 14628 | CA | PHE | G | 716 | 52.473 | 70.531 | 53.091 | 1.00 | 17.79 | 6 |
| ATOM | 14629 | CB | PHE | G | 716 | 51.230 | 71.159 | 52.467 | 1.00 | 15.42 | 6 |
| ATOM | 14630 | CG | PHE | G | 716 | 51.170 | 72.650 | 52.614 | 1.00 | 14.59 | 6 |
| ATOM | 14631 | CD1 | PHE | G | 716 | 51.584 | 73.482 | 51.583 | 1.00 | 14.85 | 6 |
| ATOM | 14632 | CD2 | PHE | G | 716 | 50.675 | 73.232 | 53.781 | 1.00 | 14.00 | 6 |
| ATOM | 14633 | CE1 | PHE | G | 716 | 51.500 | 74.876 | 51.716 | 1.00 | 14.43 | 6 |
| ATOM | 14634 | CE2 | PHE | G | 716 | 50.590 | 74.615 | 53.913 | 1.00 | 13.99 | 6 |
| ATOM | 14635 | CZ | PHE | G | 716 | 51.001 | 75.435 | 52.884 | 1.00 | 13.99 | 6 |
| ATOM | 14636 | C | PHE | G | 716 | 52.461 | 69.026 | 52.870 | 1.00 | 17.00 | 6 |
| ATOM | 14637 | O | PHE | G | 716 | 53.463 | 68.462 | 52.463 | 1.00 | 16.45 | 8 |
| ATOM | 14638 | N | GLN | G | 717 | 51.327 | 68.396 | 53.173 | 1.00 | 26.66 | 7 |
| ATOM | 14639 | CA | GLN | G | 717 | 51.111 | 66.963 | 52.979 | 1.00 | 28.92 | 6 |
| ATOM | 14640 | CB | GLN | G | 717 | 50.809 | 66.272 | 54.308 | 1.00 | 36.50 | 6 |
| ATOM | 14641 | CG | GLN | G | 717 | 52.049 | 65.885 | 55.102 | 1.00 | 40.15 | 6 |
| ATOM | 14642 | CD | GLN | G | 717 | 51.735 | 65.009 | 56.304 | 1.00 | 42.75 | 6 |
| ATOM | 14643 | OE1 | GLN | G | 717 | 50.871 | 64.136 | 56.237 | 1.00 | 45.51 | 8 |
| ATOM | 14644 | NE2 | GLN | G | 717 | 52.450 | 65.226 | 57.405 | 1.00 | 42.90 | 7 |
| ATOM | 14645 | C | GLN | G | 717 | 49.890 | 66.920 | 52.063 | 1.00 | 28.79 | 6 |
| ATOM | 14646 | O | GLN | G | 717 | 48.896 | 67.574 | 52.342 | 1.00 | 31.04 | 8 |
| ATOM | 14647 | N | PRO | G | 718 | 49.938 | 66.156 | 50.960 | 1.00 | 16.35 | 7 |
| ATOM | 14648 | CD | PRO | G | 718 | 50.992 | 65.310 | 50.391 | 1.00 | 32.39 | 6 |
| ATOM | 14649 | CA | PRO | G | 718 | 48.765 | 66.148 | 50.089 | 1.00 | 15.09 | 6 |
| ATOM | 14650 | CB | PRO | G | 718 | 49.202 | 65.287 | 48.909 | 1.00 | 30.27 | 6 |
| ATOM | 14651 | CG | PRO | G | 718 | 50.668 | 65.394 | 48.920 | 1.00 | 31.37 | 6 |
| ATOM | 14652 | C | PRO | G | 718 | 47.527 | 65.596 | 50.743 | 1.00 | 15.32 | 6 |
| ATOM | 14653 | O | PRO | G | 718 | 47.593 | 64.892 | 51.760 | 1.00 | 15.79 | 8 |
| ATOM | 14654 | N | VAL | G | 719 | 46.395 | 65.943 | 50.136 | 1.00 | 16.51 | 7 |
| ATOM | 14655 | CA | VAL | G | 719 | 45.082 | 65.501 | 50.567 | 1.00 | 17.02 | 6 |
| ATOM | 14656 | CB | VAL | G | 719 | 44.532 | 66.392 | 51.655 | 1.00 | 14.46 | 6 |
| ATOM | 14657 | CG1 | VAL | G | 719 | 43.148 | 65.949 | 52.007 | 1.00 | 14.39 | 6 |
| ATOM | 14658 | CG2 | VAL | G | 719 | 45.414 | 66.308 | 52.870 | 1.00 | 14.74 | 6 |
| ATOM | 14659 | C | VAL | G | 719 | 44.172 | 65.567 | 49.351 | 1.00 | 17.89 | 6 |
| ATOM | 14660 | O | VAL | G | 719 | 43.609 | 66.611 | 49.047 | 1.00 | 16.69 | 8 |
| ATOM | 14661 | N | LEU | G | 720 | 44.049 | 64.433 | 48.663 | 1.00 | 20.33 | 7 |
| ATOM | 14662 | CA | LEU | G | 720 | 43.241 | 64.305 | 47.460 | 1.00 | 21.11 | 6 |
| ATOM | 14663 | CB | LEU | G | 720 | 43.025 | 62.829 | 47.126 | 1.00 | 14.64 | 6 |
| ATOM | 14664 | CG | LEU | G | 720 | 44.124 | 62.364 | 46.165 | 1.00 | 13.87 | 6 |
| ATOM | 14665 | CD1 | LEU | G | 720 | 45.426 | 62.116 | 46.909 | 1.00 | 13.87 | 6 |
| ATOM | 14666 | CD2 | LEU | G | 720 | 43.669 | 61.121 | 45.462 | 1.00 | 14.34 | 6 |
| ATOM | 14667 | C | LEU | G | 720 | 41.914 | 65.048 | 47.463 | 1.00 | 22.73 | 6 |
| ATOM | 14668 | O | LEU | G | 720 | 41.010 | 64.773 | 48.258 | 1.00 | 21.44 | 8 |
| ATOM | 14669 | N | VAL | G | 721 | 41.821 | 65.988 | 46.529 | 1.00 | 39.17 | 7 |
| ATOM | 14670 | CA | VAL | G | 721 | 40.662 | 66.845 | 46.364 | 1.00 | 42.72 | 6 |
| ATOM | 14671 | CB | VAL | G | 721 | 41.084 | 68.336 | 46.571 | 1.00 | 54.49 | 6 |
| ATOM | 14672 | CG1 | VAL | G | 721 | 39.872 | 69.250 | 46.548 | 1.00 | 55.85 | 6 |
| ATOM | 14673 | CG2 | VAL | G | 721 | 41.824 | 68.491 | 47.893 | 1.00 | 55.86 | 6 |
| ATOM | 14674 | C | VAL | G | 721 | 40.043 | 66.657 | 44.971 | 1.00 | 44.38 | 6 |
| ATOM | 14675 | O | VAL | G | 721 | 40.421 | 65.758 | 44.225 | 1.00 | 44.51 | 8 |
| ATOM | 14676 | N | GLU | G | 722 | 39.101 | 67.527 | 44.628 | 1.00 | 58.78 | 7 |
| ATOM | 14677 | CA | GLU | G | 722 | 38.424 | 67.463 | 43.353 | 1.00 | 59.57 | 6 |
| ATOM | 14678 | CB | GLU | G | 722 | 37.169 | 68.324 | 43.393 | 1.00 | 85.56 | 6 |
| ATOM | 14679 | CG | GLU | G | 722 | 35.947 | 67.470 | 43.233 | 1.00 | 89.94 | 6 |
| ATOM | 14680 | CD | GLU | G | 722 | 36.237 | 66.029 | 43.613 | 1.00 | 92.32 | 6 |
| ATOM | 14681 | OE1 | GLU | G | 722 | 36.653 | 65.793 | 44.770 | 1.00 | 93.46 | 8 |
| ATOM | 14682 | OE2 | GLU | G | 722 | 36.066 | 65.141 | 42.750 | 1.00 | 93.50 | 8 |
| ATOM | 14683 | C | GLU | G | 722 | 39.214 | 67.763 | 42.090 | 1.00 | 59.65 | 6 |
| ATOM | 14684 | O | GLU | G | 722 | 40.318 | 67.241 | 41.902 | 1.00 | 61.04 | 8 |
| ATOM | 14685 | N | GLY | G | 723 | 38.645 | 68.603 | 41.226 | 1.00 | 40.93 | 7 |
| ATOM | 14686 | CA | GLY | G | 723 | 39.284 | 68.889 | 39.950 | 1.00 | 38.76 | 6 |
| ATOM | 14687 | C | GLY | G | 723 | 39.825 | 70.263 | 39.617 | 1.00 | 37.33 | 6 |
| ATOM | 14688 | O | GLY | G | 723 | 39.082 | 71.224 | 39.425 | 1.00 | 37.23 | 8 |
| ATOM | 14689 | N | GLN | G | 724 | 41.141 | 70.325 | 39.476 | 1.00 | 29.06 | 7 |
| ATOM | 14690 | CA | GLN | G | 724 | 41.822 | 71.559 | 39.176 | 1.00 | 26.74 | 6 |
| ATOM | 14691 | CB | GLN | G | 724 | 41.145 | 72.288 | 38.022 | 1.00 | 40.38 | 6 |
| ATOM | 14692 | CG | GLN | G | 724 | 41.719 | 71.923 | 36.651 | 1.00 | 42.31 | 6 |
| ATOM | 14693 | CD | GLN | G | 724 | 41.394 | 70.507 | 36.235 | 1.00 | 42.14 | 6 |
| ATOM | 14694 | OE1 | GLN | G | 724 | 41.978 | 69.967 | 35.296 | 1.00 | 40.27 | 8 |
| ATOM | 14695 | NE2 | GLN | G | 724 | 40.443 | 69.903 | 36.925 | 1.00 | 43.33 | 7 |
| ATOM | 14696 | C | GLN | G | 724 | 41.845 | 72.412 | 40.427 | 1.00 | 25.18 | 6 |
| ATOM | 14697 | O | GLN | G | 724 | 41.233 | 72.061 | 41.436 | 1.00 | 25.52 | 8 |
| ATOM | 14698 | N | SER | G | 725 | 42.563 | 73.526 | 40.356 | 1.00 | 19.49 | 7 |
| ATOM | 14699 | CA | SER | G | 725 | 42.723 | 74.435 | 41.485 | 1.00 | 17.67 | 6 |
| ATOM | 14700 | CB | SER | G | 725 | 41.448 | 75.274 | 41.705 | 1.00 | 13.87 | 6 |
| ATOM | 14701 | OG | SER | G | 725 | 40.322 | 74.505 | 42.059 | 1.00 | 13.87 | 8 |
| ATOM | 14702 | C | SER | G | 725 | 43.126 | 73.699 | 42.768 | 1.00 | 16.50 | 6 |
| ATOM | 14703 | O | SER | G | 725 | 42.358 | 72.896 | 43.299 | 1.00 | 16.70 | 8 |
| ATOM | 14704 | N | ILE | G | 726 | 44.345 | 73.952 | 43.246 | 1.00 | 20.69 | 7 |
| ATOM | 14705 | CA | ILE | G | 726 | 44.817 | 73.305 | 44.465 | 1.00 | 19.81 | 6 |
| ATOM | 14706 | CB | ILE | G | 726 | 46.353 | 73.548 | 44.721 | 1.00 | 14.20 | 6 |
| ATOM | 14707 | CG2 | ILE | G | 726 | 46.787 | 72.857 | 45.997 | 1.00 | 14.20 | 6 |
| ATOM | 14708 | CG1 | ILE | G | 726 | 47.218 | 72.976 | 43.589 | 1.00 | 14.72 | 6 |
| ATOM | 14709 | CD1 | ILE | G | 726 | 47.303 | 73.852 | 42.353 | 1.00 | 17.23 | 6 |
| ATOM | 14710 | C | ILE | G | 726 | 44.013 | 73.960 | 45.571 | 1.00 | 18.92 | 6 |
| ATOM | 14711 | O | ILE | G | 726 | 43.654 | 75.116 | 45.448 | 1.00 | 19.18 | 8 |
| ATOM | 14712 | N | GLN | G | 727 | 43.706 | 73.220 | 46.629 | 1.00 | 13.87 | 7 |
| ATOM | 14713 | CA | GLN | G | 727 | 42.950 | 73.767 | 47.749 | 1.00 | 14.48 | 6 |
| ATOM | 14714 | CB | GLN | G | 727 | 41.992 | 72.722 | 48.305 | 1.00 | 24.01 | 6 |
| ATOM | 14715 | CG | GLN | G | 727 | 40.716 | 72.603 | 47.528 | 1.00 | 23.54 | 6 |
| ATOM | 14716 | CD | GLN | G | 727 | 39.644 | 71.914 | 48.326 | 1.00 | 25.34 | 6 |
| ATOM | 14717 | OE1 | GLN | G | 727 | 38.481 | 71.856 | 47.916 | 1.00 | 25.77 | 8 |
| ATOM | 14718 | NE2 | GLN | G | 727 | 40.024 | 71.385 | 49.484 | 1.00 | 24.98 | 7 |
| ATOM | 14719 | C | GLN | G | 727 | 43.880 | 74.231 | 48.861 | 1.00 | 15.32 | 6 |
| ATOM | 14720 | O | GLN | G | 727 | 44.431 | 73.397 | 49.595 | 1.00 | 15.78 | 8 |
| ATOM | 14721 | N | LEU | G | 728 | 44.036 | 75.551 | 48.999 | 1.00 | 34.39 | 7 |
| ATOM | 14722 | CA | LEU | G | 728 | 44.936 | 76.142 | 49.998 | 1.00 | 34.99 | 6 |
| ATOM | 14723 | CB | LEU | G | 728 | 45.629 | 77.342 | 49.375 | 1.00 | 13.87 | 6 |
| ATOM | 14724 | CG | LEU | G | 728 | 46.750 | 78.040 | 50.121 | 1.00 | 13.87 | 6 |
| ATOM | 14725 | CD1 | LEU | G | 728 | 48.094 | 77.459 | 49.744 | 1.00 | 13.87 | 6 |
| ATOM | 14726 | CD2 | LEU | G | 728 | 46.684 | 79.521 | 49.760 | 1.00 | 14.94 | 6 |
| ATOM | 14727 | C | LEU | G | 728 | 44.221 | 76.547 | 51.289 | 1.00 | 36.53 | 6 |
| ATOM | 14728 | O | LEU | G | 728 | 43.030 | 76.852 | 51.260 | 1.00 | 39.24 | 8 |
| ATOM | 14729 | N | HIS | G | 729 | 44.958 | 76.569 | 52.406 | 1.00 | 28.88 | 7 |
| ATOM | 14730 | CA | HIS | G | 729 | 44.410 | 76.873 | 53.753 | 1.00 | 28.66 | 6 |
| ATOM | 14731 | CB | HIS | G | 729 | 45.146 | 76.010 | 54.768 | 1.00 | 28.50 | 6 |
| ATOM | 14732 | CG | HIS | G | 729 | 44.943 | 76.434 | 56.180 | 1.00 | 27.76 | 6 |
| ATOM | 14733 | CD2 | HIS | G | 729 | 45.810 | 76.543 | 57.210 | 1.00 | 28.21 | 6 |
| ATOM | 14734 | ND1 | HIS | G | 729 | 43.711 | 76.769 | 56.683 | 1.00 | 29.65 | 7 |
| ATOM | 14735 | CE1 | HIS | G | 729 | 43.823 | 77.062 | 57.965 | 1.00 | 29.49 | 6 |
| ATOM | 14736 | NE2 | HIS | G | 729 | 45.088 | 76.929 | 58.310 | 1.00 | 27.87 | 7 |
| ATOM | 14737 | C | HIS | G | 729 | 44.379 | 78.327 | 54.268 | 1.00 | 29.30 | 6 |
| ATOM | 14738 | O | HIS | G | 729 | 45.418 | 78.897 | 54.595 | 1.00 | 30.59 | 8 |
| ATOM | 14739 | N | PRO | G | 730 | 43.172 | 78.898 | 54.453 | 1.00 | 38.51 | 7 |
| ATOM | 14740 | CD | PRO | G | 730 | 41.999 | 78.112 | 54.848 | 1.00 | 22.47 | 6 |
| ATOM | 14741 | CA | PRO | G | 730 | 42.985 | 80.273 | 54.917 | 1.00 | 38.14 | 6 |
| ATOM | 14742 | CB | PRO | G | 730 | 41.629 | 80.230 | 55.616 | 1.00 | 20.38 | 6 |
| ATOM | 14743 | CG | PRO | G | 730 | 41.543 | 78.863 | 56.079 | 1.00 | 21.03 | 6 |
| ATOM | 14744 | C | PRO | G | 730 | 44.062 | 80.919 | 55.768 | 1.00 | 38.03 | 6 |
| ATOM | 14745 | O | PRO | G | 730 | 44.247 | 82.127 | 55.671 | 1.00 | 39.63 | 8 |
| ATOM | 14746 | N | LEU | G | 731 | 44.775 | 80.165 | 56.596 | 1.00 | 15.71 | 7 |
| ATOM | 14747 | CA | LEU | G | 731 | 45.818 | 80.814 | 57.392 | 1.00 | 14.59 | 6 |
| ATOM | 14748 | CB | LEU | G | 731 | 46.174 | 80.013 | 58.649 | 1.00 | 17.32 | 6 |
| ATOM | 14749 | CG | LEU | G | 731 | 45.183 | 80.029 | 59.815 | 1.00 | 17.32 | 6 |
| ATOM | 14750 | CD1 | LEU | G | 731 | 45.812 | 79.365 | 61.015 | 1.00 | 17.76 | 6 |
| ATOM | 14751 | CD2 | LEU | G | 731 | 44.812 | 81.449 | 60.158 | 1.00 | 17.32 | 6 |
| ATOM | 14752 | C | LEU | G | 731 | 47.077 | 81.031 | 56.567 | 1.00 | 15.18 | 6 |
| ATOM | 14753 | O | LEU | G | 731 | 47.641 | 82.129 | 56.521 | 1.00 | 15.61 | 8 |
| ATOM | 14754 | N | VAL | G | 732 | 47.523 | 79.981 | 55.905 | 1.00 | 23.13 | 7 |
| ATOM | 14755 | CA | VAL | G | 732 | 48.711 | 80.082 | 55.090 | 1.00 | 25.05 | 6 |
| ATOM | 14756 | CB | VAL | G | 732 | 48.870 | 78.821 | 54.249 | 1.00 | 18.02 | 6 |
| ATOM | 14757 | CG1 | VAL | G | 732 | 49.744 | 79.112 | 53.040 | 1.00 | 19.24 | 6 |
| ATOM | 14758 | CG2 | VAL | G | 732 | 49.466 | 77.715 | 55.108 | 1.00 | 17.58 | 6 |
| ATOM | 14759 | C | VAL | G | 732 | 48.684 | 81.317 | 54.187 | 1.00 | 26.23 | 6 |
| ATOM | 14760 | O | VAL | G | 732 | 49.691 | 81.988 | 54.011 | 1.00 | 26.14 | 8 |
| ATOM | 14761 | N | CYS | G | 733 | 47.528 | 81.629 | 53.631 | 1.00 | 29.02 | 7 |
| ATOM | 14762 | CA | CYS | G | 733 | 47.435 | 82.778 | 52.755 | 1.00 | 30.73 | 6 |
| ATOM | 14763 | CB | CYS | G | 733 | 46.038 | 83.382 | 52.829 | 1.00 | 33.46 | 6 |
| ATOM | 14764 | SG | CYS | G | 733 | 44.897 | 82.520 | 51.784 | 1.00 | 39.09 | 16 |
| ATOM | 14765 | C | CYS | G | 733 | 48.479 | 83.856 | 53.020 | 1.00 | 30.47 | 6 |
| ATOM | 14766 | O | CYS | G | 733 | 49.531 | 83.876 | 52.383 | 1.00 | 32.24 | 8 |
| ATOM | 14767 | N | GLU | G | 734 | 48.180 | 84.733 | 53.975 | 1.00 | 19.36 | 7 |
| ATOM | 14768 | CA | GLU | G | 734 | 49.028 | 85.864 | 54.357 | 1.00 | 17.14 | 6 |
| ATOM | 14769 | CB | GLU | G | 734 | 48.860 | 86.141 | 55.846 | 1.00 | 56.42 | 6 |
| ATOM | 14770 | CG | GLU | G | 734 | 49.561 | 87.391 | 56.299 | 1.00 | 60.62 | 6 |
| ATOM | 14771 | CD | GLU | G | 734 | 49.045 | 87.879 | 57.625 | 1.00 | 63.07 | 6 |
| ATOM | 14772 | OE1 | GLU | G | 734 | 47.849 | 88.241 | 57.691 | 1.00 | 62.92 | 8 |
| ATOM | 14773 | OE2 | GLU | G | 734 | 49.828 | 87.895 | 58.598 | 1.00 | 64.54 | 8 |
| ATOM | 14774 | C | GLU | G | 734 | 50.499 | 85.746 | 54.023 | 1.00 | 14.32 | 6 |
| ATOM | 14775 | O | GLU | G | 734 | 50.974 | 86.402 | 53.116 | 1.00 | 13.87 | 8 |
| ATOM | 14776 | N | ALA | G | 735 | 51.218 | 84.918 | 54.766 | 1.00 | 19.50 | 7 |
| ATOM | 14777 | CA | ALA | G | 735 | 52.630 | 84.731 | 54.524 | 1.00 | 18.84 | 6 |
| ATOM | 14778 | CB | ALA | G | 735 | 53.089 | 83.451 | 55.136 | 1.00 | 52.86 | 6 |
| ATOM | 14779 | C | ALA | G | 735 | 52.867 | 84.698 | 53.040 | 1.00 | 19.82 | 6 |
| ATOM | 14780 | O | ALA | G | 735 | 53.674 | 85.471 | 52.544 | 1.00 | 21.91 | 8 |
| ATOM | 14781 | N | PHE | G | 736 | 52.147 | 83.811 | 52.341 | 1.00 | 19.80 | 7 |
| ATOM | 14782 | CA | PHE | G | 736 | 52.236 | 83.618 | 50.865 | 1.00 | 19.91 | 6 |
| ATOM | 14783 | CB | PHE | G | 736 | 51.409 | 82.416 | 50.394 | 1.00 | 39.31 | 6 |
| ATOM | 14784 | CG | PHE | G | 736 | 51.977 | 81.077 | 50.761 | 1.00 | 41.46 | 6 |
| ATOM | 14785 | CD1 | PHE | G | 736 | 52.558 | 80.850 | 52.013 | 1.00 | 43.08 | 6 |
| ATOM | 14786 | CD2 | PHE | G | 736 | 51.850 | 80.012 | 49.881 | 1.00 | 41.14 | 6 |
| ATOM | 14787 | CE1 | PHE | G | 736 | 52.998 | 79.575 | 52.384 | 1.00 | 43.64 | 6 |
| ATOM | 14788 | CE2 | PHE | G | 736 | 52.280 | 78.749 | 50.236 | 1.00 | 42.45 | 6 |
| ATOM | 14789 | CZ | PHE | G | 736 | 52.856 | 78.527 | 51.494 | 1.00 | 42.53 | 6 |
| ATOM | 14790 | C | PHE | G | 736 | 51.667 | 84.793 | 50.117 | 1.00 | 18.63 | 6 |
| ATOM | 14791 | O | PHE | G | 736 | 52.102 | 85.119 | 49.028 | 1.00 | 18.17 | 8 |
| ATOM | 14792 | N | ASN | G | 737 | 50.662 | 85.403 | 50.721 | 1.00 | 19.99 | 7 |
| ATOM | 14793 | CA | ASN | G | 737 | 49.949 | 86.514 | 50.131 | 1.00 | 22.37 | 6 |
| ATOM | 14794 | CB | ASN | G | 737 | 50.877 | 87.656 | 49.691 | 1.00 | 28.87 | 6 |
| ATOM | 14795 | CG | ASN | C | 737 | 50.097 | 88.878 | 49.168 | 1.00 | 30.91 | 6 |
| ATOM | 14796 | OD1 | ASN | G | 737 | 49.308 | 88.769 | 48.218 | 1.00 | 31.45 | 8 |
| ATOM | 14797 | ND2 | ASN | G | 737 | 50.309 | 90.035 | 49.793 | 1.00 | 30.32 | 7 |
| ATOM | 14798 | C | ASN | G | 737 | 49.277 | 85.924 | 48.920 | 1.00 | 22.93 | 6 |
| ATOM | 14799 | O | ASN | G | 737 | 49.951 | 85.516 | 47.972 | 1.00 | 23.26 | 8 |
| ATOM | 14800 | N | ALA | G | 738 | 47.949 | 85.881 | 48.946 | 1.00 | 41.86 | 7 |
| ATOM | 14801 | CA | ALA | G | 738 | 47.236 | 85.321 | 47.826 | 1.00 | 42.97 | 6 |
| ATOM | 14802 | CB | ALA | G | 738 | 47.570 | 83.841 | 47.724 | 1.00 | 39.26 | 6 |
| ATOM | 14803 | C | ALA | G | 738 | 45.747 | 85.480 | 47.934 | 1.00 | 43.00 | 6 |
| ATOM | 14804 | O | ALA | G | 738 | 45.103 | 84.559 | 48.379 | 1.00 | 44.08 | 8 |
| ATOM | 14805 | N | ASP | G | 739 | 45.160 | 86.603 | 47.547 | 1.00 | 25.53 | 7 |
| ATOM | 14806 | CA | ASP | G | 739 | 43.705 | 86.612 | 47.670 | 1.00 | 26.98 | 6 |
| ATOM | 14807 | CB | ASP | G | 739 | 43.087 | 88.033 | 47.761 | 1.00 | 31.33 | 6 |
| ATOM | 14808 | CG | ASP | G | 739 | 42.968 | 88.730 | 46.433 | 1.00 | 31.96 | 6 |
| ATOM | 14809 | OD1 | ASP | G | 739 | 43.985 | 89.271 | 45.959 | 1.00 | 34.21 | 8 |
| ATOM | 14810 | OD2 | ASP | G | 739 | 41.854 | 88.748 | 45.870 | 1.00 | 29.43 | 8 |
| ATOM | 14811 | C | ASP | G | 739 | 43.133 | 85.797 | 46.517 | 1.00 | 27.24 | 6 |
| ATOM | 14812 | O | ASP | G | 739 | 43.441 | 86.016 | 45.348 | 1.00 | 27.57 | 8 |
| ATOM | 14813 | N | PHE | G | 740 | 42.326 | 84.813 | 46.876 | 1.00 | 27.06 | 7 |
| ATOM | 14814 | CA | PHE | G | 740 | 41.742 | 83.926 | 45.906 | 1.00 | 26.36 | 6 |
| ATOM | 14815 | CB | PHE | G | 740 | 40.870 | 82.885 | 46.610 | 1.00 | 15.95 | 6 |
| ATOM | 14816 | CG | PHE | G | 740 | 41.599 | 82.076 | 47.651 | 1.00 | 13.87 | 6 |
| ATOM | 14817 | CD1 | PHE | G | 740 | 41.584 | 82.459 | 48.977 | 1.00 | 13.87 | 6 |
| ATOM | 14818 | CD2 | PHE | G | 740 | 42.279 | 80.927 | 47.306 | 1.00 | 13.87 | 6 |
| ATOM | 14819 | CE1 | PHE | G | 740 | 42.230 | 81.722 | 49.932 | 1.00 | 13.87 | 6 |
| ATOM | 14820 | CE2 | PHE | G | 740 | 42.924 | 80.193 | 48.259 | 1.00 | 13.87 | 6 |
| ATOM | 14821 | CZ | PHE | G | 740 | 42.898 | 80.591 | 49.579 | 1.00 | 13.87 | 6 |
| ATOM | 14822 | C | PHE | G | 740 | 40.919 | 84.624 | 44.848 | 1.00 | 27.47 | 6 |
| ATOM | 14823 | O | PHE | G | 740 | 39.696 | 84.558 | 44.886 | 1.00 | 28.22 | 8 |
| ATOM | 14824 | N | ASP | G | 741 | 41.567 | 85.289 | 43.900 | 1.00 | 13.87 | 7 |
| ATOM | 14825 | CA | ASP | G | 741 | 40.804 | 85.938 | 42.848 | 1.00 | 13.87 | 6 |
| ATOM | 14826 | CB | ASP | G | 741 | 40.612 | 87.429 | 43.122 | 1.00 | 59.38 | 6 |
| ATOM | 14827 | CG | ASP | G | 741 | 41.897 | 88.196 | 43.102 | 1.00 | 63.10 | 6 |
| ATOM | 14828 | OD1 | ASP | G | 741 | 42.910 | 87.623 | 42.651 | 1.00 | 65.86 | 8 |
| ATOM | 14829 | OD2 | ASP | G | 741 | 41.883 | 89.375 | 43.530 | 1.00 | 64.67 | 8 |
| ATOM | 14830 | C | ASP | G | 741 | 41.446 | 85.749 | 41.503 | 1.00 | 13.87 | 6 |
| ATOM | 14831 | O | ASP | G | 741 | 41.435 | 86.647 | 40.665 | 1.00 | 13.87 | 8 |
| ATOM | 14832 | N | GLY | G | 742 | 41.990 | 84.552 | 41.306 | 1.00 | 39.80 | 7 |
| ATOM | 14833 | CA | GLY | G | 742 | 42.645 | 84.208 | 40.059 | 1.00 | 41.21 | 6 |
| ATOM | 14834 | C | GLY | G | 742 | 44.089 | 83.790 | 40.265 | 1.00 | 41.72 | 6 |
| ATOM | 14835 | O | GLY | G | 742 | 44.799 | 83.499 | 39.308 | 1.00 | 41.86 | 8 |
| ATOM | 14836 | N | ASP | G | 743 | 44.527 | 83.748 | 41.516 | 1.00 | 23.67 | 7 |
| ATOM | 14837 | CA | ASP | G | 743 | 45.902 | 83.382 | 41.804 | 1.00 | 23.48 | 6 |
| ATOM | 14838 | CB | ASP | G | 743 | 46.229 | 83.625 | 43.276 | 1.00 | 42.73 | 6 |
| ATOM | 14839 | CG | ASP | G | 743 | 46.325 | 85.094 | 43.613 | 1.00 | 44.71 | 6 |
| ATOM | 14840 | OD1 | ASP | G | 743 | 46.874 | 85.850 | 42.782 | 1.00 | 46.33 | 8 |
| ATOM | 14841 | OD2 | ASP | G | 743 | 45.876 | 85.487 | 44.713 | 1.00 | 44.74 | 8 |
| ATOM | 14842 | C | ASP | G | 743 | 46.243 | 81.950 | 41.447 | 1.00 | 22.67 | 6 |
| ATOM | 14843 | O | ASP | G | 743 | 45.377 | 81.102 | 41.346 | 1.00 | 22.59 | 8 |
| ATOM | 14844 | N | GLN | G | 744 | 47.526 | 81.693 | 41.253 | 1.00 | 42.42 | 7 |
| ATOM | 14845 | CA | GLN | G | 744 | 47.992 | 80.366 | 40.919 | 1.00 | 41.78 | 6 |
| ATOM | 14846 | CB | GLN | G | 744 | 48.379 | 80.290 | 39.458 | 1.00 | 42.22 | 6 |
| ATOM | 14847 | CG | GLN | G | 744 | 47.269 | 80.439 | 38.494 | 1.00 | 43.50 | 6 |
| ATOM | 14848 | CD | GLN | G | 744 | 47.612 | 79.718 | 37.221 | 1.00 | 45.81 | 6 |
| ATOM | 14849 | OE1 | GLN | G | 744 | 47.743 | 78.498 | 37.217 | 1.00 | 45.52 | 8 |
| ATOM | 14850 | NE2 | GLN | G | 744 | 47.784 | 80.461 | 36.135 | 1.00 | 48.73 | 7 |
| ATOM | 14851 | C | GLN | G | 744 | 49.221 | 80.048 | 41.746 | 1.00 | 40.15 | 6 |
| ATOM | 14852 | O | GLN | G | 744 | 50.001 | 80.944 | 42.073 | 1.00 | 40.06 | 8 |
| ATOM | 14853 | N | MET | G | 745 | 49.396 | 78.771 | 42.071 | 1.00 | 13.87 | 7 |
| ATOM | 14854 | CA | MET | G | 745 | 50.539 | 78.343 | 42.844 | 1.00 | 13.87 | 6 |
| ATOM | 14855 | CB | MET | G | 745 | 50.118 | 77.959 | 44.264 | 1.00 | 14.30 | 6 |
| ATOM | 14856 | CG | MET | G | 745 | 50.036 | 79.178 | 45.179 | 1.00 | 14.30 | 6 |
| ATOM | 14857 | SD | MET | G | 745 | 49.811 | 78.869 | 46.948 | 1.00 | 16.68 | 16 |
| ATOM | 14858 | CE | MET | G | 745 | 47.967 | 79.149 | 47.112 | 1.00 | 14.30 | 6 |
| ATOM | 14859 | C | MET | G | 745 | 51.164 | 77.182 | 42.134 | 1.00 | 13.87 | 6 |
| ATOM | 14860 | O | MET | G | 745 | 50.503 | 76.535 | 41.336 | 1.00 | 13.87 | 8 |
| ATOM | 14861 | N | ALA | G | 746 | 52.449 | 76.952 | 42.384 | 1.00 | 16.13 | 7 |
| ATOM | 14862 | CA | ALA | G | 746 | 53.153 | 75.832 | 41.780 | 1.00 | 18.41 | 6 |
| ATOM | 14863 | CB | ALA | G | 746 | 54.607 | 76.155 | 41.638 | 1.00 | 13.87 | 6 |
| ATOM | 14864 | C | ALA | G | 746 | 52.948 | 74.658 | 42.739 | 1.00 | 19.55 | 6 |
| ATOM | 14865 | O | ALA | G | 746 | 52.069 | 74.729 | 43.598 | 1.00 | 20.61 | 8 |
| ATOM | 14866 | N | VAL | G | 747 | 53.725 | 73.583 | 42.633 | 1.00 | 30.45 | 7 |
| ATOM | 14867 | CA | VAL | G | 747 | 53.484 | 72.472 | 43.551 | 1.00 | 30.58 | 6 |
| ATOM | 14868 | CB | VAL | G | 747 | 52.094 | 71.826 | 43.235 | 1.00 | 23.22 | 6 |
| ATOM | 14869 | CG1 | VAL | G | 747 | 52.163 | 71.029 | 41.937 | 1.00 | 23.65 | 6 |
| ATOM | 14870 | CG2 | VAL | G | 747 | 51.630 | 70.976 | 44.392 | 1.00 | 23.96 | 6 |
| ATOM | 14871 | C | VAL | G | 747 | 54.554 | 71.379 | 43.631 | 1.00 | 30.28 | 6 |
| ATOM | 14872 | O | VAL | G | 747 | 54.245 | 70.243 | 43.948 | 1.00 | 32.28 | 8 |
| ATOM | 14873 | N | HIS | G | 748 | 55.808 | 71.726 | 43.376 | 1.00 | 17.28 | 7 |
| ATOM | 14874 | CA | HIS | G | 748 | 56.924 | 70.772 | 43.431 | 1.00 | 17.23 | 6 |
| ATOM | 14875 | CB | HIS | G | 748 | 58.216 | 71.518 | 43.722 | 1.00 | 13.87 | 6 |
| ATOM | 14876 | CG | HIS | G | 748 | 58.360 | 72.760 | 42.921 | 1.00 | 13.87 | 6 |
| ATOM | 14877 | CD2 | HIS | G | 748 | 58.186 | 74.059 | 43.256 | 1.00 | 13.87 | 6 |
| ATOM | 14878 | ND1 | HIS | G | 748 | 58.592 | 72.738 | 41.566 | 1.00 | 13.87 | 7 |
| ATOM | 14879 | CE1 | HIS | G | 748 | 58.550 | 73.972 | 41.097 | 1.00 | 13.87 | 6 |
| ATOM | 14880 | NE2 | HIS | G | 748 | 58.303 | 74.792 | 42.103 | 1.00 | 13.87 | 7 |
| ATOM | 14881 | C | HIS | G | 748 | 56.798 | 69.643 | 44.439 | 1.00 | 18.83 | 6 |
| ATOM | 14882 | O | HIS | G | 748 | 56.114 | 69.760 | 45.445 | 1.00 | 20.21 | 8 |
| ATOM | 14883 | N | VAL | G | 749 | 57.499 | 68.552 | 44.170 | 1.00 | 25.03 | 7 |
| ATOM | 14884 | CA | VAL | G | 749 | 57.486 | 67.395 | 45.047 | 1.00 | 26.18 | 6 |
| ATOM | 14885 | CB | VAL | G | 749 | 56.884 | 66.176 | 44.325 | 1.00 | 39.04 | 6 |
| ATOM | 14886 | CG1 | VAL | G | 749 | 56.855 | 64.978 | 45.260 | 1.00 | 38.12 | 6 |
| ATOM | 14887 | CG2 | VAL | G | 749 | 55.507 | 66.501 | 43.809 | 1.00 | 40.76 | 6 |
| ATOM | 14888 | C | VAL | G | 749 | 58.918 | 67.020 | 45.436 | 1.00 | 27.56 | 6 |
| ATOM | 14889 | O | VAL | G | 749 | 59.729 | 66.755 | 44.553 | 1.00 | 28.62 | 8 |
| ATOM | 14890 | N | PRO | G | 750 | 59.265 | 67.021 | 46.746 | 1.00 | 22.28 | 7 |
| ATOM | 14891 | CD | PRO | G | 750 | 58.541 | 67.396 | 47.971 | 1.00 | 17.65 | 6 |
| ATOM | 14892 | CA | PRO | G | 750 | 60.645 | 66.633 | 47.061 | 1.00 | 22.70 | 6 |
| ATOM | 14893 | CB | PRO | G | 750 | 60.723 | 66.788 | 48.589 | 1.00 | 17.39 | 6 |
| ATOM | 14894 | CG | PRO | G | 750 | 59.320 | 66.656 | 49.039 | 1.00 | 17.73 | 6 |
| ATOM | 14895 | C | PRO | G | 750 | 60.784 | 65.193 | 46.566 | 1.00 | 23.59 | 6 |
| ATOM | 14896 | O | PRO | G | 750 | 59.841 | 64.395 | 46.657 | 1.00 | 23.30 | 8 |
| ATOM | 14897 | N | LEU | G | 751 | 61.952 | 64.866 | 46.032 | 1.00 | 58.94 | 7 |
| ATOM | 14898 | CA | LEU | G | 751 | 62.143 | 63.557 | 45.439 | 1.00 | 60.91 | 6 |
| ATOM | 14899 | CB | LEU | G | 751 | 62.309 | 63.747 | 43.935 | 1.00 | 52.80 | 6 |
| ATOM | 14900 | CG | LEU | G | 751 | 62.109 | 62.554 | 43.016 | 1.00 | 55.13 | 6 |
| ATOM | 14901 | CD1 | LEU | G | 751 | 60.636 | 62.428 | 42.674 | 1.00 | 54.73 | 6 |
| ATOM | 14902 | CD2 | LEU | G | 751 | 62.934 | 62.751 | 41.760 | 1.00 | 54.76 | 6 |
| ATOM | 14903 | C | LEU | G | 751 | 63.302 | 62.723 | 45.959 | 1.00 | 61.93 | 6 |
| ATOM | 14904 | O | LEU | G | 751 | 63.539 | 61.626 | 45.458 | 1.00 | 63.68 | 8 |
| ATOM | 14905 | N | SER | G | 752 | 64.025 | 63.218 | 46.957 | 1.00 | 43.89 | 7 |
| ATOM | 14906 | CA | SER | G | 752 | 65.171 | 62.476 | 47.479 | 1.00 | 42.39 | 6 |
| ATOM | 14907 | CB | SER | G | 752 | 66.460 | 63.167 | 47.028 | 1.00 | 31.08 | 6 |
| ATOM | 14908 | OG | SER | G | 752 | 66.202 | 64.486 | 46.543 | 1.00 | 28.73 | 8 |
| ATOM | 14909 | C | SER | G | 752 | 65.192 | 62.301 | 48.991 | 1.00 | 42.78 | 6 |
| ATOM | 14910 | O | SER | G | 752 | 65.996 | 62.931 | 49.657 | 1.00 | 43.79 | 8 |
| ATOM | 14911 | N | SER | G | 753 | 64.321 | 61.439 | 49.517 | 1.00 | 26.53 | 7 |
| ATOM | 14912 | CA | SER | G | 753 | 64.231 | 61.172 | 50.959 | 1.00 | 26.13 | 6 |
| ATOM | 14913 | CB | SER | G | 753 | 64.985 | 59.903 | 51.320 | 1.00 | 29.71 | 6 |
| ATOM | 14914 | OG | SER | G | 753 | 65.030 | 59.763 | 52.721 | 1.00 | 27.27 | 8 |
| ATOM | 14915 | C | SER | G | 753 | 64.759 | 62.312 | 51.804 | 1.00 | 26.42 | 6 |
| ATOM | 14916 | O | SER | G | 753 | 63.997 | 62.995 | 52.471 | 1.00 | 26.57 | 8 |
| ATOM | 14917 | N | PHE | G | 754 | 66.072 | 62.493 | 51.793 | 1.00 | 36.84 | 7 |
| ATOM | 14918 | CA | PHE | G | 754 | 66.709 | 63.587 | 52.512 | 1.00 | 38.63 | 6 |
| ATOM | 14919 | CB | PHE | G | 754 | 68.147 | 63.775 | 51.997 | 1.00 | 47.55 | 6 |
| ATOM | 14920 | CG | PHE | G | 754 | 69.144 | 64.147 | 53.061 | 1.00 | 49.17 | 6 |
| ATOM | 14921 | CD1 | PHE | G | 754 | 70.449 | 63.677 | 52.996 | 1.00 | 49.98 | 6 |
| ATOM | 14922 | CD2 | PHE | G | 754 | 68.778 | 64.934 | 54.141 | 1.00 | 50.57 | 6 |
| ATOM | 14923 | CE1 | PHE | G | 754 | 71.370 | 63.978 | 53.996 | 1.00 | 50.17 | 6 |
| ATOM | 14924 | CE2 | PHE | G | 754 | 69.697 | 65.239 | 55.147 | 1.00 | 51.20 | 6 |
| ATOM | 14925 | CZ | PHE | G | 754 | 70.993 | 64.756 | 55.071 | 1.00 | 50.82 | 6 |
| ATOM | 14926 | C | PHE | G | 754 | 65.887 | 64.845 | 52.206 | 1.00 | 39.18 | 6 |
| ATOM | 14927 | O | PHE | G | 754 | 65.482 | 65.566 | 53.114 | 1.00 | 39.46 | 8 |
| ATOM | 14928 | N | ALA | G | 755 | 65.625 | 65.078 | 50.921 | 1.00 | 30.87 | 7 |
| ATOM | 14929 | CA | ALA | G | 755 | 64.868 | 66.243 | 50.463 | 1.00 | 31.35 | 6 |
| ATOM | 14930 | CB | ALA | G | 755 | 64.803 | 66.260 | 48.943 | 1.00 | 58.86 | 6 |
| ATOM | 14931 | C | ALA | G | 755 | 63.465 | 66.289 | 51.027 | 1.00 | 31.07 | 6 |
| ATOM | 14932 | O | ALA | G | 755 | 62.848 | 67.347 | 51.106 | 1.00 | 31.27 | 8 |
| ATOM | 14933 | N | GLN | G | 756 | 62.958 | 65.128 | 51.404 | 1.00 | 24.89 | 7 |
| ATOM | 14934 | CA | GLN | G | 756 | 61.625 | 65.025 | 51.957 | 1.00 | 24.71 | 6 |
| ATOM | 14935 | CB | GLN | G | 756 | 61.111 | 63.598 | 51.746 | 1.00 | 24.28 | 6 |
| ATOM | 14936 | CG | GLN | G | 756 | 59.671 | 63.346 | 52.145 | 1.00 | 24.69 | 6 |
| ATOM | 14937 | CD | GLN | G | 756 | 58.695 | 64.009 | 51.208 | 1.00 | 25.03 | 6 |
| ATOM | 14938 | OE1 | GLN | G | 756 | 58.925 | 64.069 | 50.002 | 1.00 | 24.26 | 8 |
| ATOM | 14939 | NE2 | GLN | G | 756 | 57.585 | 64.492 | 51.748 | 1.00 | 26.11 | 7 |
| ATOM | 14940 | C | GLN | G | 756 | 61.709 | 65.358 | 53.448 | 1.00 | 25.94 | 6 |
| ATOM | 14941 | O | GLN | G | 756 | 60.991 | 66.222 | 53.953 | 1.00 | 25.96 | 8 |
| ATOM | 14942 | N | ALA | G | 757 | 62.608 | 64.675 | 54.146 | 1.00 | 51.89 | 7 |
| ATOM | 14943 | CA | ALA | G | 757 | 62.765 | 64.876 | 55.575 | 1.00 | 52.36 | 6 |
| ATOM | 14944 | CB | ALA | G | 757 | 63.846 | 63.973 | 56.126 | 1.00 | 33.89 | 6 |
| ATOM | 14945 | C | ALA | G | 757 | 63.090 | 66.308 | 55.900 | 1.00 | 52.19 | 6 |
| ATOM | 14946 | O | ALA | G | 757 | 62.688 | 66.799 | 56.944 | 1.00 | 52.90 | 8 |
| ATOM | 14947 | N | GLU | G | 758 | 63.809 | 66.990 | 55.019 | 1.00 | 39.08 | 7 |
| ATOM | 14948 | CA | GLU | G | 758 | 64.162 | 68.373 | 55.313 | 1.00 | 39.81 | 6 |
| ATOM | 14949 | CB | GLU | G | 758 | 65.576 | 68.712 | 54.805 | 1.00 | 54.36 | 6 |
| ATOM | 14950 | CG | GLU | G | 758 | 65.834 | 68.472 | 53.329 | 1.00 | 58.27 | 6 |
| ATOM | 14951 | CD | GLU | G | 758 | 67.213 | 68.949 | 52.906 | 1.00 | 59.69 | 6 |
| ATOM | 14952 | OE1 | GLU | G | 758 | 68.185 | 68.697 | 53.652 | 1.00 | 60.05 | 8 |
| ATOM | 14953 | OE2 | GLU | G | 758 | 67.327 | 69.566 | 51.825 | 1.00 | 61.43 | 8 |
| ATOM | 14954 | C | GLU | G | 758 | 63.150 | 69.372 | 54.789 | 1.00 | 38.43 | 6 |
| ATOM | 14955 | O | GLU | G | 758 | 63.213 | 70.555 | 55.092 | 1.00 | 39.11 | 8 |
| ATOM | 14956 | N | ALA | G | 759 | 62.206 | 68.907 | 53.999 | 1.00 | 30.91 | 7 |
| ATOM | 14957 | CA | ALA | G | 759 | 61.202 | 69.823 | 53.519 | 1.00 | 30.09 | 6 |
| ATOM | 14958 | CB | ALA | G | 759 | 60.930 | 69.593 | 52.057 | 1.00 | 40.69 | 6 |
| ATOM | 14959 | C | ALA | G | 759 | 59.956 | 69.566 | 54.341 | 1.00 | 30.03 | 6 |
| ATOM | 14960 | O | ALA | G | 759 | 58.867 | 69.952 | 53.941 | 1.00 | 28.91 | 8 |
| ATOM | 14961 | N | ARG | G | 760 | 60.133 | 68.901 | 55.483 | 1.00 | 50.71 | 7 |
| ATOM | 14962 | CA | ARG | G | 760 | 59.027 | 68.591 | 56.389 | 1.00 | 51.90 | 6 |
| ATOM | 14963 | CB | ARG | G | 760 | 58.376 | 67.252 | 56.015 | 1.00 | 69.53 | 6 |
| ATOM | 14964 | CG | ARG | G | 760 | 59.276 | 66.045 | 56.186 | 1.00 | 73.92 | 6 |
| ATOM | 14965 | CD | ARG | G | 760 | 58.511 | 64.739 | 56.035 | 1.00 | 77.04 | 6 |
| ATOM | 14966 | NE | ARG | G | 760 | 57.452 | 64.577 | 57.029 | 1.00 | 80.23 | 7 |
| ATOM | 14967 | CZ | ARG | G | 760 | 56.795 | 63.437 | 57.206 | 1.00 | 82.53 | 6 |
| ATOM | 14968 | NH1 | ARG | G | 760 | 57.103 | 62.391 | 56.457 | 1.00 | 81.99 | 7 |
| ATOM | 14969 | NH2 | ARG | G | 760 | 55.839 | 63.330 | 58.119 | 1.00 | 84.32 | 7 |
| ATOM | 14970 | C | ARG | G | 760 | 59.445 | 68.560 | 57.870 | 1.00 | 51.26 | 6 |
| ATOM | 14971 | O | ARG | G | 760 | 58.798 | 67.920 | 58.702 | 1.00 | 51.59 | 8 |
| ATOM | 14972 | N | ILE | G | 761 | 60.533 | 69.247 | 58.192 | 1.00 | 75.37 | 7 |
| ATOM | 14973 | CA | ILE | G | 761 | 61.030 | 69.334 | 59.561 | 1.00 | 73.54 | 6 |
| ATOM | 14974 | CB | ILE | G | 761 | 62.199 | 68.341 | 59.812 | 1.00 | 18.31 | 6 |
| ATOM | 14975 | CG2 | ILE | G | 761 | 63.146 | 68.851 | 60.906 | 1.00 | 17.14 | 6 |
| ATOM | 14976 | CG1 | ILE | G | 761 | 61.624 | 66.981 | 60.185 | 1.00 | 15.32 | 6 |
| ATOM | 14977 | CD1 | ILE | G | 761 | 62.660 | 66.013 | 60.667 | 1.00 | 14.10 | 6 |
| ATOM | 14978 | C | ILE | G | 761 | 61.516 | 70.760 | 59.688 | 1.00 | 73.70 | 6 |
| ATOM | 14979 | O | ILE | G | 761 | 61.585 | 71.333 | 60.770 | 1.00 | 74.84 | 8 |
| ATOM | 14980 | N | GLN | G | 762 | 61.829 | 71.340 | 58.547 | 1.00 | 33.05 | 7 |
| ATOM | 14981 | CA | GLN | G | 762 | 62.296 | 72.695 | 58.529 | 1.00 | 32.66 | 6 |
| ATOM | 14982 | CB | GLN | G | 762 | 63.647 | 72.785 | 57.828 | 1.00 | 25.09 | 6 |
| ATOM | 14983 | CG | GLN | G | 762 | 64.372 | 71.438 | 57.709 | 1.00 | 25.21 | 6 |
| ATOM | 14984 | CD | GLN | G | 762 | 65.869 | 71.568 | 57.457 | 1.00 | 24.08 | 6 |
| ATOM | 14985 | OE1 | GLN | G | 762 | 66.326 | 72.311 | 56.573 | 1.00 | 21.31 | 8 |
| ATOM | 14986 | NE2 | GLN | G | 762 | 66.637 | 70.834 | 58.238 | 1.00 | 22.16 | 7 |
| ATOM | 14987 | C | GLN | G | 762 | 61.299 | 73.574 | 57.821 | 1.00 | 32.37 | 6 |
| ATOM | 14988 | O | GLN | G | 762 | 61.004 | 74.646 | 58.323 | 1.00 | 33.51 | 8 |
| ATOM | 14989 | N | MET | G | 763 | 60.761 | 73.131 | 56.678 | 1.00 | 19.86 | 7 |
| ATOM | 14990 | CA | MET | G | 763 | 59.813 | 73.963 | 55.929 | 1.00 | 18.23 | 6 |
| ATOM | 14991 | CB | MET | G | 763 | 60.396 | 74.329 | 54.552 | 1.00 | 32.01 | 6 |
| ATOM | 14992 | CG | MET | G | 763 | 60.883 | 73.195 | 53.672 | 1.00 | 33.31 | 6 |
| ATOM | 14993 | SD | MET | G | 763 | 61.784 | 73.838 | 52.207 | 1.00 | 36.23 | 16 |
| ATOM | 14994 | CE | MET | G | 763 | 60.664 | 73.538 | 50.886 | 1.00 | 36.79 | 6 |
| ATOM | 14995 | C | MET | G | 763 | 58.347 | 73.571 | 55.761 | 1.00 | 17.68 | 6 |
| ATOM | 14996 | O | MET | G | 763 | 57.710 | 73.994 | 54.808 | 1.00 | 16.85 | 8 |
| ATOM | 14997 | N | LEU | G | 764 | 57.800 | 72.793 | 56.691 | 1.00 | 34.27 | 7 |
| ATOM | 14998 | CA | LEU | G | 764 | 56.380 | 72.400 | 56.651 | 1.00 | 33.31 | 6 |
| ATOM | 14999 | CB | LEU | G | 764 | 56.139 | 71.158 | 57.515 | 1.00 | 13.87 | 6 |
| ATOM | 15000 | CG | LEU | G | 764 | 54.721 | 71.047 | 58.039 | 1.00 | 13.87 | 6 |
| ATOM | 15001 | CD1 | LEU | G | 764 | 53.813 | 70.768 | 56.908 | 1.00 | 13.87 | 6 |
| ATOM | 15002 | CD2 | LEU | G | 764 | 54.618 | 69.952 | 59.034 | 1.00 | 13.87 | 6 |
| ATOM | 15003 | C | LEU | G | 764 | 55.545 | 73.557 | 57.196 | 1.00 | 33.08 | 6 |
| ATOM | 15004 | O | LEU | G | 764 | 55.859 | 74.123 | 58.239 | 1.00 | 32.01 | 8 |
| ATOM | 15005 | N | SER | G | 765 | 54.469 | 73.906 | 56.516 | 1.00 | 41.12 | 7 |
| ATOM | 15006 | CA | SER | G | 765 | 53.680 | 75.025 | 56.992 | 1.00 | 42.05 | 6 |
| ATOM | 15007 | CB | SER | G | 765 | 52.407 | 75.205 | 56.157 | 1.00 | 15.01 | 6 |
| ATOM | 15008 | OG | SER | G | 765 | 51.395 | 74.276 | 56.484 | 1.00 | 13.87 | 8 |
| ATOM | 15009 | C | SER | G | 765 | 53.305 | 74.919 | 58.455 | 1.00 | 43.52 | 6 |
| ATOM | 15010 | O | SER | G | 765 | 53.170 | 75.933 | 59.133 | 1.00 | 45.29 | 8 |
| ATOM | 15011 | N | ALA | G | 766 | 53.159 | 73.700 | 58.953 | 1.00 | 15.35 | 7 |
| ATOM | 15012 | CA | ALA | G | 766 | 52.753 | 73.506 | 60.342 | 1.00 | 17.16 | 6 |
| ATOM | 15013 | CB | ALA | G | 766 | 52.348 | 72.050 | 60.564 | 1.00 | 120.56 | 6 |
| ATOM | 15014 | C | ALA | G | 766 | 53.791 | 73.918 | 61.386 | 1.00 | 18.99 | 6 |
| ATOM | 15015 | O | ALA | G | 766 | 53.437 | 74.151 | 62.548 | 1.00 | 17.29 | 8 |
| ATOM | 15016 | N | HIS | G | 767 | 55.064 | 73.991 | 60.992 | 1.00 | 66.55 | 7 |
| ATOM | 15017 | CA | HIS | G | 767 | 56.117 | 74.376 | 61.930 | 1.00 | 68.12 | 6 |
| ATOM | 15018 | CB | HIS | G | 767 | 57.291 | 73.388 | 61.908 | 1.00 | 38.10 | 6 |
| ATOM | 15019 | CG | HIS | G | 767 | 56.904 | 71.953 | 61.729 | 1.00 | 40.34 | 6 |
| ATOM | 15020 | CD2 | HIS | G | 767 | 57.309 | 71.033 | 60.820 | 1.00 | 39.96 | 6 |
| ATOM | 15021 | ND1 | HIS | G | 767 | 56.044 | 71.295 | 62.581 | 1.00 | 41.67 | 7 |
| ATOM | 15022 | CE1 | HIS | G | 767 | 55.936 | 70.033 | 62.204 | 1.00 | 41.91 | 6 |
| ATOM | 15023 | NE2 | HIS | G | 767 | 56.695 | 69.849 | 61.139 | 1.00 | 41.62 | 7 |
| ATOM | 15024 | C | HIS | G | 767 | 56.694 | 75.755 | 61.606 | 1.00 | 69.05 | 6 |
| ATOM | 15025 | O | HIS | G | 767 | 56.382 | 76.765 | 62.254 | 1.00 | 68.74 | 8 |
| ATOM | 15026 | N | ASN | G | 768 | 57.550 | 75.779 | 60.590 | 1.00 | 37.53 | 7 |
| ATOM | 15027 | CA | ASN | G | 768 | 58.225 | 77.004 | 60.185 | 1.00 | 38.30 | 6 |
| ATOM | 15028 | CB | ASN | G | 768 | 59.317 | 76.685 | 59.161 | 1.00 | 98.05 | 6 |
| ATOM | 15029 | CG | ASN | G | 768 | 60.362 | 77.783 | 59.058 | 1.00 | 101.19 | 6 |
| ATOM | 15030 | OD1 | ASN | G | 768 | 60.138 | 78.818 | 58.429 | 1.00 | 103.81 | 8 |
| ATOM | 15031 | ND2 | ASN | G | 768 | 61.511 | 77.564 | 59.694 | 1.00 | 103.05 | 7 |
| ATOM | 15032 | C | ASN | G | 768 | 57.293 | 78.064 | 59.636 | 1.00 | 36.76 | 6 |
| ATOM | 15033 | O | ASN | G | 768 | 57.619 | 78.753 | 58.668 | 1.00 | 35.44 | 8 |
| ATOM | 15034 | N | LEU | G | 769 | 56.131 | 78.193 | 60.259 | 1.00 | 56.17 | 7 |
| ATOM | 15035 | CA | LEU | G | 769 | 55.176 | 79.196 | 59.836 | 1.00 | 57.04 | 6 |
| ATOM | 15036 | CB | LEU | G | 769 | 53.864 | 78.549 | 59.388 | 1.00 | 112.18 | 6 |
| ATOM | 15037 | CG | LEU | G | 769 | 53.270 | 79.078 | 58.072 | 1.00 | 113.89 | 6 |
| ATOM | 15038 | CD1 | LEU | G | 769 | 52.035 | 78.269 | 57.700 | 1.00 | 113.61 | 6 |
| ATOM | 15039 | CD2 | LEU | G | 769 | 52.928 | 80.562 | 58.206 | 1.00 | 114.19 | 6 |
| ATOM | 15040 | C | LEU | G | 769 | 54.955 | 80.047 | 61.061 | 1.00 | 56.62 | 6 |
| ATOM | 15041 | O | LEU | G | 769 | 55.168 | 81.256 | 61.044 | 1.00 | 57.60 | 8 |
| ATOM | 15042 | N | LEU | G | 770 | 54.555 | 79.392 | 62.137 | 1.00 | 73.72 | 7 |
| ATOM | 15043 | CA | LEU | G | 770 | 54.310 | 80.077 | 63.390 | 1.00 | 71.62 | 6 |
| ATOM | 15044 | CB | LEU | G | 770 | 54.007 | 79.047 | 64.481 | 1.00 | 29.35 | 6 |
| ATOM | 15045 | CG | LEU | G | 770 | 52.758 | 78.190 | 64.292 | 1.00 | 27.37 | 6 |
| ATOM | 15046 | CD1 | LEU | G | 770 | 52.853 | 77.386 | 63.004 | 1.00 | 26.30 | 6 |
| ATOM | 15047 | CD2 | LEU | G | 770 | 52.618 | 77.287 | 65.487 | 1.00 | 26.49 | 6 |
| ATOM | 15048 | C | LEU | G | 770 | 55.530 | 80.916 | 63.776 | 1.00 | 70.73 | 6 |
| ATOM | 15049 | O | LEU | G | 770 | 56.644 | 80.388 | 63.882 | 1.00 | 70.99 | 8 |
| ATOM | 15050 | N | SER | G | 771 | 55.316 | 82.215 | 63.981 | 1.00 | 27.71 | 7 |
| ATOM | 15051 | CA | SER | G | 771 | 56.400 | 83.122 | 64.353 | 1.00 | 25.01 | 6 |
| ATOM | 15052 | CB | SER | G | 771 | 57.049 | 82.650 | 65.650 | 1.00 | 13.87 | 6 |
| ATOM | 15053 | OG | SER | G | 771 | 56.082 | 82.467 | 66.658 | 1.00 | 13.87 | 8 |
| ATOM | 15054 | C | SER | G | 771 | 57.424 | 83.114 | 63.222 | 1.00 | 26.50 | 6 |
| ATOM | 15055 | O | SER | G | 771 | 58.329 | 82.288 | 63.213 | 1.00 | 26.59 | 8 |
| ATOM | 15056 | N | PRO | G | 772 | 57.292 | 84.042 | 62.258 | 1.00 | 27.42 | 7 |
| ATOM | 15057 | CD | PRO | G | 772 | 56.066 | 84.831 | 62.076 | 1.00 | 128.55 | 6 |
| ATOM | 15058 | CA | PRO | G | 772 | 58.143 | 84.214 | 61.079 | 1.00 | 27.59 | 6 |
| ATOM | 15059 | CB | PRO | G | 772 | 57.135 | 84.535 | 60.016 | 1.00 | 129.03 | 6 |
| ATOM | 15060 | CG | PRO | G | 772 | 56.353 | 85.558 | 60.769 | 1.00 | 129.81 | 6 |
| ATOM | 15061 | C | PRO | G | 772 | 59.165 | 85.330 | 61.146 | 1.00 | 27.08 | 6 |
| ATOM | 15062 | O | PRO | G | 772 | 58.833 | 86.451 | 61.521 | 1.00 | 24.70 | 8 |
| ATOM | 15063 | N | ALA | G | 773 | 60.375 | 85.008 | 60.695 | 1.00 | 56.69 | 7 |
| ATOM | 15064 | CA | ALA | G | 773 | 61.524 | 85.915 | 60.652 | 1.00 | 58.46 | 6 |
| ATOM | 15065 | CB | ALA | G | 773 | 62.233 | 85.734 | 59.347 | 1.00 | 13.87 | 6 |
| ATOM | 15066 | C | ALA | G | 773 | 61.281 | 87.412 | 60.903 | 1.00 | 60.27 | 6 |
| ATOM | 15067 | O | ALA | G | 773 | 60.992 | 88.175 | 59.968 | 1.00 | 61.67 | 8 |
| ATOM | 15068 | N | SER | G | 774 | 61.438 | 87.792 | 62.182 | 1.00 | 33.09 | 7 |
| ATOM | 15069 | CA | SER | G | 774 | 61.279 | 89.150 | 62.740 | 1.00 | 29.64 | 6 |
| ATOM | 15070 | CB | SER | G | 774 | 61.263 | 90.200 | 61.625 | 1.00 | 53.29 | 6 |
| ATOM | 15071 | OG | SER | G | 774 | 62.431 | 90.074 | 60.828 | 1.00 | 52.85 | 8 |
| ATOM | 15072 | C | SER | G | 774 | 60.069 | 89.325 | 63.690 | 1.00 | 27.63 | 6 |
| ATOM | 15073 | O | SER | G | 774 | 59.120 | 90.071 | 63.414 | 1.00 | 24.23 | 8 |
| ATOM | 15074 | N | GLY | G | 775 | 60.128 | 88.580 | 64.796 | 1.00 | 62.93 | 7 |
| ATOM | 15075 | CA | GLY | G | 775 | 59.122 | 88.664 | 65.841 | 1.00 | 63.64 | 6 |
| ATOM | 15076 | C | GLY | G | 775 | 57.859 | 87.819 | 65.969 | 1.00 | 63.06 | 6 |
| ATOM | 15077 | O | GLY | G | 775 | 57.899 | 86.630 | 66.322 | 1.00 | 62.24 | 8 |
| ATOM | 15078 | N | GLU | G | 776 | 56.737 | 88.497 | 65.708 | 1.00 | 27.39 | 7 |
| ATOM | 15079 | CA | GLU | G | 776 | 55.366 | 87.991 | 65.807 | 1.00 | 27.23 | 6 |
| ATOM | 15080 | CB | GLU | G | 776 | 54.381 | 89.125 | 65.494 | 1.00 | 60.04 | 6 |
| ATOM | 15081 | CG | GLU | G | 776 | 55.003 | 90.468 | 65.196 | 1.00 | 63.57 | 6 |
| ATOM | 15082 | CD | GLU | G | 776 | 54.732 | 91.461 | 66.299 | 1.00 | 66.43 | 6 |
| ATOM | 15083 | OE1 | GLU | G | 776 | 53.545 | 91.606 | 66.652 | 1.00 | 67.74 | 8 |
| ATOM | 15084 | OE2 | GLU | G | 776 | 55.690 | 92.091 | 66.810 | 1.00 | 69.61 | 8 |
| ATOM | 15085 | C | GLU | G | 776 | 54.921 | 86.791 | 64.997 | 1.00 | 24.15 | 6 |
| ATOM | 15086 | O | GLU | G | 776 | 55.639 | 86.298 | 64.147 | 1.00 | 23.57 | 8 |
| ATOM | 15087 | N | PRO | G | 777 | 53.701 | 86.303 | 65.273 | 1.00 | 26.96 | 7 |
| ATOM | 15088 | CD | PRO | G | 777 | 52.823 | 86.626 | 66.413 | 1.00 | 13.87 | 6 |
| ATOM | 15089 | CA | PRO | G | 777 | 53.171 | 85.161 | 64.541 | 1.00 | 26.04 | 6 |
| ATOM | 15090 | CB | PRO | G | 777 | 51.975 | 84.754 | 65.390 | 1.00 | 13.87 | 6 |
| ATOM | 15091 | CG | PRO | G | 777 | 51.533 | 86.037 | 65.983 | 1.00 | 13.87 | 6 |
| ATOM | 15092 | C | PRO | G | 777 | 52.763 | 85.608 | 63.142 | 1.00 | 25.66 | 6 |
| ATOM | 15093 | O | PRO | G | 777 | 52.037 | 86.579 | 62.991 | 1.00 | 25.44 | 8 |
| ATOM | 15094 | N | LEU | G | 778 | 53.240 | 84.915 | 62.119 | 1.00 | 21.61 | 7 |
| ATOM | 15095 | CA | LEU | G | 778 | 52.882 | 85.265 | 60.757 | 1.00 | 22.03 | 6 |
| ATOM | 15096 | CB | LEU | G | 778 | 53.896 | 84.714 | 59.766 | 1.00 | 13.87 | 6 |
| ATOM | 15097 | CG | LEU | G | 778 | 53.949 | 85.275 | 58.354 | 1.00 | 13.87 | 6 |
| ATOM | 15098 | CD1 | LEU | G | 778 | 52.606 | 85.907 | 58.074 | 1.00 | 13.87 | 6 |
| ATOM | 15099 | CD2 | LEU | G | 778 | 55.059 | 86.298 | 58.184 | 1.00 | 13.87 | 6 |
| ATOM | 15100 | C | LEU | G | 778 | 51.555 | 84.585 | 60.517 | 1.00 | 24.64 | 6 |
| ATOM | 15101 | O | LEU | G | 778 | 50.676 | 85.144 | 59.860 | 1.00 | 25.71 | 8 |
| ATOM | 15102 | N | ALA | G | 779 | 51.435 | 83.368 | 61.058 | 1.00 | 36.17 | 7 |
| ATOM | 15103 | CA | ALA | G | 779 | 50.227 | 82.541 | 60.952 | 1.00 | 36.16 | 6 |
| ATOM | 15104 | CB | ALA | G | 779 | 50.592 | 81.050 | 61.005 | 1.00 | 14.38 | 6 |
| ATOM | 15105 | C | ALA | G | 779 | 49.277 | 82.875 | 62.088 | 1.00 | 35.75 | 6 |
| ATOM | 15106 | O | ALA | G | 779 | 49.321 | 82.261 | 63.149 | 1.00 | 35.75 | 8 |
| ATOM | 15107 | N | LYS | G | 780 | 48.414 | 83.849 | 61.862 | 1.00 | 23.74 | 7 |
| ATOM | 15108 | CA | LYS | G | 780 | 47.492 | 84.242 | 62.900 | 1.00 | 24.26 | 6 |
| ATOM | 15109 | CB | LYS | G | 780 | 47.791 | 85.667 | 63.342 | 1.00 | 29.93 | 6 |
| ATOM | 15110 | CG | LYS | G | 780 | 47.427 | 86.705 | 62.292 | 1.00 | 31.31 | 6 |
| ATOM | 15111 | CD | LYS | G | 780 | 48.046 | 88.057 | 62.595 | 1.00 | 32.06 | 6 |
| ATOM | 15112 | CE | LYS | G | 780 | 49.556 | 88.013 | 62.446 | 1.00 | 32.04 | 6 |
| ATOM | 15113 | NZ | LYS | G | 780 | 49.938 | 87.535 | 61.086 | 1.00 | 30.99 | 7 |
| ATOM | 15114 | C | LYS | G | 780 | 46.123 | 84.194 | 62.280 | 1.00 | 23.69 | 6 |
| ATOM | 15115 | O | LYS | G | 780 | 46.017 | 84.166 | 61.048 | 1.00 | 24.61 | 8 |
| ATOM | 15116 | N | PRO | G | 781 | 45.055 | 84.161 | 63.114 | 1.00 | 20.33 | 7 |
| ATOM | 15117 | CD | PRO | G | 781 | 45.042 | 83.954 | 64.573 | 1.00 | 13.87 | 6 |
| ATOM | 15118 | CA | PRO | G | 781 | 43.693 | 84.124 | 62.573 | 1.00 | 19.24 | 6 |
| ATOM | 15119 | CB | PRO | G | 781 | 42.820 | 84.011 | 63.823 | 1.00 | 13.87 | 6 |
| ATOM | 15120 | CG | PRO | G | 781 | 43.693 | 84.461 | 64.942 | 1.00 | 13.87 | 6 |
| ATOM | 15121 | C | PRO | G | 781 | 43.490 | 85.411 | 61.812 | 1.00 | 17.87 | 6 |
| ATOM | 15122 | O | PRO | G | 781 | 44.144 | 86.404 | 62.122 | 1.00 | 17.15 | 8 |
| ATOM | 15123 | N | SER | G | 782 | 42.613 | 85.412 | 60.820 | 1.00 | 15.44 | 7 |
| ATOM | 15124 | CA | SER | G | 782 | 42.439 | 86.633 | 60.064 | 1.00 | 17.28 | 6 |
| ATOM | 15125 | CB | SER | G | 782 | 43.510 | 86.724 | 58.995 | 1.00 | 60.50 | 6 |
| ATOM | 15126 | OG | SER | G | 782 | 43.341 | 85.682 | 58.050 | 1.00 | 65.71 | 8 |
| ATOM | 15127 | C | SER | G | 782 | 41.088 | 86.817 | 59.406 | 1.00 | 18.24 | 6 |
| ATOM | 15128 | O | SER | G | 782 | 40.214 | 85.970 | 59.525 | 1.00 | 18.07 | 8 |
| ATOM | 15129 | N | ALA | G | 783 | 40.952 | 87.948 | 58.711 | 1.00 | 45.23 | 7 |
| ATOM | 15130 | CA | ALA | G | 783 | 39.751 | 88.379 | 57.990 | 1.00 | 46.50 | 6 |
| ATOM | 15131 | CB | ALA | G | 783 | 40.169 | 88.948 | 56.626 | 1.00 | 37.16 | 6 |
| ATOM | 15132 | C | ALA | G | 783 | 38.647 | 87.345 | 57.795 | 1.00 | 46.84 | 6 |
| ATOM | 15133 | O | ALA | G | 783 | 38.633 | 86.670 | 56.772 | 1.00 | 47.76 | 8 |
| ATOM | 15134 | N | ASP | G | 784 | 37.722 | 87.248 | 58.753 | 1.00 | 36.67 | 7 |
| ATOM | 15135 | CA | ASP | G | 784 | 36.586 | 86.305 | 58.719 | 1.00 | 37.55 | 6 |
| ATOM | 15136 | CB | ASP | G | 784 | 36.595 | 85.430 | 57.456 | 1.00 | 49.98 | 6 |
| ATOM | 15137 | CG | ASP | G | 784 | 35.200 | 85.233 | 56.861 | 1.00 | 51.36 | 6 |
| ATOM | 15138 | OD1 | ASP | G | 784 | 34.285 | 84.851 | 57.612 | 1.00 | 52.12 | 8 |
| ATOM | 15139 | OD2 | ASP | G | 784 | 35.013 | 85.447 | 55.641 | 1.00 | 52.33 | 8 |
| ATOM | 15140 | C | ASP | G | 784 | 36.615 | 85.408 | 59.947 | 1.00 | 37.15 | 6 |
| ATOM | 15141 | O | ASP | G | 784 | 35.579 | 85.005 | 60.470 | 1.00 | 38.29 | 8 |
| ATOM | 15142 | N | ILE | G | 785 | 37.815 | 85.076 | 60.393 | 1.00 | 41.46 | 7 |
| ATOM | 15143 | CA | ILE | G | 785 | 37.964 | 84.264 | 61.577 | 1.00 | 39.70 | 6 |
| ATOM | 15144 | CB | ILE | G | 785 | 39.382 | 83.693 | 61.696 | 1.00 | 15.05 | 6 |
| ATOM | 15145 | CG2 | ILE | G | 785 | 39.507 | 82.869 | 62.970 | 1.00 | 14.47 | 6 |
| ATOM | 15146 | CG1 | ILE | G | 785 | 39.700 | 82.871 | 60.452 | 1.00 | 15.58 | 6 |
| ATOM | 15147 | CD1 | ILE | G | 785 | 40.778 | 81.822 | 60.660 | 1.00 | 17.48 | 6 |
| ATOM | 15148 | C | ILE | G | 785 | 37.748 | 85.247 | 62.706 | 1.00 | 39.86 | 6 |
| ATOM | 15149 | O | ILE | G | 785 | 36.954 | 85.030 | 63.611 | 1.00 | 40.10 | 8 |
| ATOM | 15150 | N | ILE | G | 786 | 38.454 | 86.358 | 62.632 | 1.00 | 24.73 | 7 |
| ATOM | 15151 | CA | ILE | G | 786 | 38.349 | 87.359 | 63.661 | 1.00 | 23.79 | 6 |
| ATOM | 15152 | CB | ILE | G | 786 | 39.362 | 88.467 | 63.418 | 1.00 | 15.20 | 6 |
| ATOM | 15153 | CG2 | ILE | G | 786 | 39.425 | 89.404 | 64.594 | 1.00 | 16.33 | 6 |
| ATOM | 15154 | CG1 | ILE | G | 786 | 40.737 | 87.876 | 63.226 | 1.00 | 14.70 | 6 |
| ATOM | 15155 | CD1 | ILE | G | 786 | 41.661 | 88.878 | 62.603 | 1.00 | 18.15 | 6 |
| ATOM | 15156 | C | ILE | G | 786 | 36.949 | 87.954 | 63.663 | 1.00 | 23.11 | 6 |
| ATOM | 15157 | O | ILE | G | 786 | 36.513 | 88.534 | 64.656 | 1.00 | 21.58 | 8 |
| ATOM | 15158 | N | LEU | G | 787 | 36.231 | 87.811 | 62.556 | 1.00 | 38.69 | 7 |
| ATOM | 15159 | CA | LEU | G | 787 | 34.886 | 88.378 | 62.478 | 1.00 | 38.51 | 6 |
| ATOM | 15160 | CB | LEU | G | 787 | 34.371 | 88.324 | 61.045 | 1.00 | 19.03 | 6 |
| ATOM | 15161 | CG | LEU | G | 787 | 33.002 | 88.954 | 60.859 | 1.00 | 18.73 | 6 |
| ATOM | 15162 | CD1 | LEU | G | 787 | 33.040 | 90.379 | 61.357 | 1.00 | 18.88 | 6 |
| ATOM | 15163 | CD2 | LEU | G | 787 | 32.622 | 88.911 | 59.391 | 1.00 | 18.73 | 6 |
| ATOM | 15164 | C | LEU | G | 787 | 33.918 | 87.648 | 63.386 | 1.00 | 38.98 | 6 |
| ATOM | 15165 | O | LEU | G | 787 | 33.109 | 88.263 | 64.086 | 1.00 | 38.48 | 8 |
| ATOM | 15166 | N | GLY | G | 788 | 34.018 | 86.325 | 63.356 | 1.00 | 37.13 | 7 |
| ATOM | 15167 | CA | GLY | G | 788 | 33.163 | 85.488 | 64.164 | 1.00 | 37.11 | 6 |
| ATOM | 15168 | C | GLY | G | 788 | 33.623 | 85.461 | 65.599 | 1.00 | 37.38 | 6 |
| ATOM | 15169 | O | GLY | G | 788 | 32.801 | 85.588 | 66.495 | 1.00 | 38.29 | 8 |
| ATOM | 15170 | N | LEU | G | 789 | 34.926 | 85.311 | 65.827 | 1.00 | 19.19 | 7 |
| ATOM | 15171 | CA | LEU | G | 789 | 35.446 | 85.256 | 67.184 | 1.00 | 19.28 | 6 |
| ATOM | 15172 | CB | LEU | G | 789 | 36.948 | 85.004 | 67.164 | 1.00 | 13.87 | 6 |
| ATOM | 15173 | CG | LEU | G | 789 | 37.247 | 83.521 | 67.008 | 1.00 | 13.87 | 6 |
| ATOM | 15174 | CD1 | LEU | G | 789 | 38.616 | 83.204 | 67.546 | 1.00 | 13.87 | 6 |
| ATOM | 15175 | CD2 | LEU | G | 789 | 36.217 | 82.736 | 67.781 | 1.00 | 13.87 | 6 |
| ATOM | 15176 | C | LEU | G | 789 | 35.135 | 86.489 | 68.016 | 1.00 | 20.25 | 6 |
| ATOM | 15177 | O | LEU | G | 789 | 35.162 | 86.459 | 69.242 | 1.00 | 19.66 | 8 |
| ATOM | 15178 | N | TYR | G | 790 | 34.829 | 87.584 | 67.347 | 1.00 | 32.63 | 7 |
| ATOM | 15179 | CA | TYR | G | 790 | 34.511 | 88.803 | 68.051 | 1.00 | 32.68 | 6 |
| ATOM | 15180 | CB | TYR | G | 790 | 34.905 | 89.994 | 67.155 | 1.00 | 20.56 | 6 |
| ATOM | 15181 | CG | TYR | G | 790 | 34.730 | 91.382 | 67.736 | 1.00 | 19.81 | 6 |
| ATOM | 15182 | CD1 | TYR | G | 790 | 33.483 | 92.005 | 67.727 | 1.00 | 22.03 | 6 |
| ATOM | 15183 | CE1 | TYR | G | 790 | 33.307 | 93.301 | 68.204 | 1.00 | 22.46 | 6 |
| ATOM | 15184 | CD2 | TYR | G | 790 | 35.801 | 92.087 | 68.243 | 1.00 | 17.80 | 6 |
| ATOM | 15185 | CE2 | TYR | G | 790 | 35.635 | 93.383 | 68.722 | 1.00 | 19.02 | 6 |
| ATOM | 15186 | CZ | TYR | G | 790 | 34.383 | 93.993 | 68.700 | 1.00 | 21.73 | 6 |
| ATOM | 15187 | OH | TYR | G | 790 | 34.192 | 95.295 | 69.150 | 1.00 | 20.21 | 8 |
| ATOM | 15188 | C | TYR | G | 790 | 33.010 | 88.744 | 68.398 | 1.00 | 32.40 | 6 |
| ATOM | 15189 | O | TYR | G | 790 | 32.654 | 88.781 | 69.570 | 1.00 | 32.10 | 8 |
| ATOM | 15190 | N | TYR | G | 791 | 32.139 | 88.595 | 67.402 | 1.00 | 47.89 | 7 |
| ATOM | 15191 | CA | TYR | G | 791 | 30.695 | 88.537 | 67.649 | 1.00 | 50.05 | 6 |
| ATOM | 15192 | CB | TYR | G | 791 | 29.949 | 88.107 | 66.396 | 1.00 | 23.78 | 6 |
| ATOM | 15193 | CG | TYR | G | 791 | 29.028 | 89.154 | 65.827 | 1.00 | 21.98 | 6 |
| ATOM | 15194 | CD1 | TYR | G | 791 | 29.541 | 90.288 | 65.219 | 1.00 | 21.56 | 6 |
| ATOM | 15195 | CE1 | TYR | G | 791 | 28.705 | 91.235 | 64.624 | 1.00 | 21.65 | 6 |
| ATOM | 15196 | CD2 | TYR | G | 791 | 27.647 | 88.983 | 65.842 | 1.00 | 19.64 | 6 |
| ATOM | 15197 | CE2 | TYR | G | 791 | 26.790 | 89.928 | 65.251 | 1.00 | 21.32 | 6 |
| ATOM | 15198 | CZ | TYR | G | 791 | 27.331 | 91.058 | 64.635 | 1.00 | 21.76 | 6 |
| ATOM | 15199 | OH | TYR | G | 791 | 26.539 | 92.001 | 63.995 | 1.00 | 18.83 | 8 |
| ATOM | 15200 | C | TYR | G | 791 | 30.257 | 87.609 | 68.780 | 1.00 | 52.37 | 6 |
| ATOM | 15201 | O | TYR | G | 791 | 29.189 | 87.799 | 69.365 | 1.00 | 53.82 | 8 |
| ATOM | 15202 | N | ILE | G | 792 | 31.053 | 86.589 | 69.071 | 1.00 | 30.73 | 7 |
| ATOM | 15203 | CA | ILE | G | 792 | 30.719 | 85.650 | 70.138 | 1.00 | 32.10 | 6 |
| ATOM | 15204 | CB | ILE | G | 792 | 31.011 | 84.186 | 69.690 | 1.00 | 100.62 | 6 |
| ATOM | 15205 | CG2 | ILE | G | 792 | 30.773 | 83.222 | 70.825 | 1.00 | 102.54 | 6 |
| ATOM | 15206 | CG1 | ILE | G | 792 | 30.070 | 83.778 | 68.556 | 1.00 | 103.50 | 6 |
| ATOM | 15207 | CD1 | ILE | G | 792 | 30.211 | 84.589 | 67.292 | 1.00 | 106.63 | 6 |
| ATOM | 15208 | C | ILE | G | 792 | 31.600 | 86.032 | 71.319 | 1.00 | 31.58 | 6 |
| ATOM | 15209 | O | ILE | G | 792 | 32.148 | 85.177 | 72.011 | 1.00 | 33.20 | 8 |
| ATOM | 15210 | N | THR | G | 793 | 31.732 | 87.336 | 71.543 | 1.00 | 27.56 | 7 |
| ATOM | 15211 | CA | THR | G | 793 | 32.596 | 87.848 | 72.601 | 1.00 | 26.55 | 6 |
| ATOM | 15212 | CB | THR | G | 793 | 34.116 | 87.721 | 72.218 | 1.00 | 21.55 | 6 |
| ATOM | 15213 | OG1 | THR | G | 793 | 34.337 | 86.565 | 71.415 | 1.00 | 19.90 | 8 |
| ATOM | 15214 | CG2 | THR | G | 793 | 34.979 | 87.608 | 73.438 | 1.00 | 20.39 | 6 |
| ATOM | 15215 | C | THR | G | 793 | 32.359 | 89.343 | 72.814 | 1.00 | 27.96 | 6 |
| ATOM | 15216 | O | THR | G | 793 | 33.289 | 90.048 | 73.193 | 1.00 | 28.18 | 8 |
| ATOM | 15217 | N | GLN | G | 794 | 31.161 | 89.864 | 72.588 | 1.00 | 17.12 | 7 |
| ATOM | 15218 | CA | GLN | G | 794 | 31.033 | 91.296 | 72.774 | 1.00 | 19.48 | 6 |
| ATOM | 15219 | CB | GLN | G | 794 | 30.623 | 91.964 | 71.468 | 1.00 | 39.11 | 6 |
| ATOM | 15220 | CG | GLN | G | 794 | 31.618 | 93.025 | 71.016 | 1.00 | 41.22 | 6 |
| ATOM | 15221 | CD | GLN | G | 794 | 32.035 | 93.996 | 72.140 | 1.00 | 42.55 | 6 |
| ATOM | 15222 | OE1 | GLN | G | 794 | 32.500 | 93.569 | 73.203 | 1.00 | 43.82 | 8 |
| ATOM | 15223 | NE2 | GLN | G | 794 | 31.883 | 95.307 | 71.898 | 1.00 | 42.28 | 7 |
| ATOM | 15224 | C | GLN | G | 794 | 30.159 | 91.828 | 73.892 | 1.00 | 21.63 | 6 |
| ATOM | 15225 | O | GLN | G | 794 | 30.366 | 92.939 | 74.354 | 1.00 | 20.54 | 8 |
| ATOM | 15226 | N | VAL | G | 795 | 29.205 | 91.047 | 74.363 | 1.00 | 61.89 | 7 |
| ATOM | 15227 | CA | VAL | G | 795 | 28.301 | 91.531 | 75.401 | 1.00 | 65.70 | 6 |
| ATOM | 15228 | CB | VAL | G | 795 | 29.035 | 92.249 | 76.559 | 1.00 | 13.87 | 6 |
| ATOM | 15229 | CG1 | VAL | G | 795 | 28.044 | 92.598 | 77.646 | 1.00 | 13.87 | 6 |
| ATOM | 15230 | CG2 | VAL | G | 795 | 30.146 | 91.401 | 77.103 | 1.00 | 13.87 | 6 |
| ATOM | 15231 | C | VAL | G | 795 | 27.449 | 92.556 | 74.681 | 1.00 | 70.25 | 6 |
| ATOM | 15232 | O | VAL | G | 795 | 27.701 | 92.876 | 73.520 | 1.00 | 72.46 | 8 |
| ATOM | 15233 | N | ARG | G | 796 | 26.440 | 93.077 | 75.355 | 1.00 | 35.14 | 7 |
| ATOM | 15234 | CA | ARG | G | 796 | 25.603 | 94.066 | 74.710 | 1.00 | 39.99 | 6 |
| ATOM | 15235 | CB | ARG | G | 796 | 24.281 | 93.426 | 74.283 | 1.00 | 38.80 | 6 |
| ATOM | 15236 | CG | ARG | G | 796 | 24.416 | 91.981 | 73.866 | 1.00 | 40.77 | 6 |
| ATOM | 15237 | CD | ARG | G | 796 | 23.875 | 91.744 | 72.472 | 1.00 | 43.43 | 6 |
| ATOM | 15238 | NE | ARG | G | 796 | 22.545 | 91.143 | 72.458 | 1.00 | 48.09 | 7 |
| ATOM | 15239 | CZ | ARG | G | 796 | 21.994 | 90.595 | 71.376 | 1.00 | 50.25 | 6 |
| ATOM | 15240 | NH1 | ARG | G | 796 | 22.660 | 90.572 | 70.226 | 1.00 | 49.68 | 7 |
| ATOM | 15241 | NH2 | ARG | G | 796 | 20.778 | 90.070 | 71.438 | 1.00 | 53.16 | 7 |
| ATOM | 15242 | C | ARG | G | 796 | 25.340 | 95.249 | 75.640 | 1.00 | 42.90 | 6 |
| ATOM | 15243 | O | ARG | G | 796 | 25.840 | 95.308 | 76.772 | 1.00 | 43.38 | 8 |
| ATOM | 15244 | N | ALA | G | 797 | 24.553 | 96.197 | 75.151 | 1.00 | 75.68 | 7 |
| ATOM | 15245 | CA | ALA | G | 797 | 24.211 | 97.371 | 75.929 | 1.00 | 79.83 | 6 |
| ATOM | 15246 | CB | ALA | G | 797 | 25.294 | 98.429 | 75.770 | 1.00 | 92.75 | 6 |
| ATOM | 15247 | C | ALA | G | 797 | 22.863 | 97.897 | 75.452 | 1.00 | 82.83 | 6 |
| ATOM | 15248 | O | ALA | G | 797 | 22.703 | 99.088 | 75.202 | 1.00 | 83.48 | 8 |
| ATOM | 15249 | N | GLU | G | 798 | 21.899 | 96.993 | 75.316 | 1.00 | 160.01 | 7 |
| ATOM | 15250 | CA | GLU | G | 798 | 20.560 | 97.360 | 74.872 | 1.00 | 163.85 | 6 |
| ATOM | 15251 | CB | GLU | G | 798 | 19.584 | 96.212 | 75.158 | 1.00 | 76.15 | 6 |
| ATOM | 15252 | CG | GLU | G | 798 | 19.689 | 95.632 | 76.568 | 1.00 | 78.05 | 6 |
| ATOM | 15253 | CD | GLU | G | 798 | 20.062 | 94.158 | 76.570 | 1.00 | 78.36 | 6 |
| ATOM | 15254 | OE1 | GLU | G | 798 | 19.388 | 93.384 | 75.855 | 1.00 | 78.95 | 8 |
| ATOM | 15255 | OE2 | GLU | G | 798 | 21.020 | 93.776 | 77.287 | 1.00 | 78.32 | 8 |
| ATOM | 15256 | C | GLU | G | 798 | 20.090 | 98.644 | 75.555 | 1.00 | 165.80 | 6 |
| ATOM | 15257 | O | GLU | G | 798 | 19.324 | 99.422 | 74.978 | 1.00 | 167.14 | 8 |
| ATOM | 15258 | N | ALA | G | 799 | 20.563 | 98.855 | 76.783 | 1.00 | 97.67 | 7 |
| ATOM | 15259 | CA | ALA | G | 799 | 20.231 | 100.038 | 77.574 | 1.00 | 98.55 | 6 |
| ATOM | 15260 | CB | ALA | G | 799 | 21.049 | 101.209 | 77.093 | 1.00 | 13.87 | 6 |
| ATOM | 15261 | C | ALA | G | 799 | 18.752 | 100.419 | 77.566 | 1.00 | 99.18 | 6 |
| ATOM | 15262 | O | ALA | G | 799 | 18.309 | 101.153 | 76.686 | 1.00 | 100.21 | 8 |
| ATOM | 15263 | N | ALA | G | 800 | 17.995 | 99.926 | 78.544 | 1.00 | 139.95 | 7 |
| ATOM | 15264 | CA | ALA | G | 800 | 16.572 | 100.246 | 78.641 | 1.00 | 139.56 | 6 |
| ATOM | 15265 | CB | ALA | G | 800 | 15.706 | 99.010 | 78.351 | 1.00 | 29.89 | 6 |
| ATOM | 15266 | C | ALA | G | 800 | 16.289 | 100.761 | 80.040 | 1.00 | 139.22 | 6 |
| ATOM | 15267 | O | ALA | G | 800 | 15.296 | 100.395 | 80.662 | 1.00 | 140.45 | 8 |
| ATOM | 15268 | N | GLY | G | 801 | 17.181 | 101.607 | 80.536 | 1.00 | 64.62 | 7 |
| ATOM | 15269 | CA | GLY | G | 801 | 16.999 | 102.165 | 81.860 | 1.00 | 63.95 | 6 |
| ATOM | 15270 | C | GLY | G | 801 | 18.034 | 101.688 | 82.853 | 1.00 | 63.60 | 6 |
| ATOM | 15271 | O | GLY | G | 801 | 18.453 | 100.534 | 82.803 | 1.00 | 64.35 | 8 |
| ATOM | 15272 | N | ALA | G | 802 | 18.452 | 102.571 | 83.756 | 1.00 | 61.70 | 7 |
| ATOM | 15273 | CA | ALA | G | 802 | 19.437 | 102.198 | 84.761 | 1.00 | 61.19 | 6 |
| ATOM | 15274 | CB | ALA | G | 802 | 20.769 | 102.859 | 84.435 | 1.00 | 19.01 | 6 |
| ATOM | 15275 | C | ALA | G | 802 | 18.977 | 102.539 | 86.195 | 1.00 | 61.23 | 6 |
| ATOM | 15276 | O | ALA | G | 802 | 19.633 | 103.292 | 86.930 | 1.00 | 61.12 | 8 |
| ATOM | 15277 | N | GLY | G | 803 | 17.847 | 101.942 | 86.582 | 1.00 | 34.86 | 7 |
| ATOM | 15278 | CA | GLY | G | 803 | 17.264 | 102.158 | 87.897 | 1.00 | 34.00 | 6 |
| ATOM | 15279 | C | GLY | G | 803 | 17.956 | 101.593 | 89.135 | 1.00 | 33.82 | 6 |
| ATOM | 15280 | O | GLY | G | 803 | 19.181 | 101.457 | 89.189 | 1.00 | 32.87 | 8 |
| ATOM | 15281 | N | ALA | G | 804 | 17.143 | 101.270 | 90.142 | 1.00 | 86.31 | 7 |
| ATOM | 15282 | CA | ALA | G | 804 | 17.621 | 100.749 | 91.421 | 1.00 | 87.01 | 6 |
| ATOM | 15283 | CB | ALA | G | 804 | 17.369 | 101.777 | 92.489 | 1.00 | 16.35 | 6 |
| ATOM | 15284 | C | ALA | G | 804 | 16.988 | 99.416 | 91.828 | 1.00 | 88.19 | 6 |
| ATOM | 15285 | O | ALA | G | 804 | 16.063 | 98.930 | 91.177 | 1.00 | 88.92 | 8 |
| ATOM | 15286 | N | ALA | G | 805 | 17.478 | 98.833 | 92.917 | 1.00 | 105.80 | 7 |
| ATOM | 15287 | CA | ALA | G | 805 | 16.956 | 97.549 | 93.378 | 1.00 | 107.76 | 6 |
| ATOM | 15288 | CB | ALA | G | 805 | 17.444 | 96.432 | 92.450 | 1.00 | 110.30 | 6 |
| ATOM | 15289 | C | ALA | G | 805 | 17.356 | 97.237 | 94.818 | 1.00 | 108.92 | 6 |
| ATOM | 15290 | O | ALA | G | 805 | 16.500 | 97.105 | 95.701 | 1.00 | 109.43 | 8 |
| ATOM | 15291 | N | ALA | G | 806 | 18.664 | 97.095 | 95.024 | 1.00 | 128.12 | 7 |
| ATOM | 15292 | CA | ALA | G | 806 | 19.260 | 96.808 | 96.325 | 1.00 | 128.33 | 6 |
| ATOM | 15293 | CB | ALA | G | 806 | 18.490 | 97.527 | 97.435 | 1.00 | 147.98 | 6 |
| ATOM | 15294 | C | ALA | G | 806 | 19.401 | 95.328 | 96.666 | 1.00 | 128.39 | 6 |
| ATOM | 15295 | O | ALA | G | 806 | 20.503 | 94.783 | 96.595 | 1.00 | 128.67 | 8 |
| ATOM | 15296 | N | ALA | G | 807 | 18.289 | 94.683 | 97.023 | 1.00 | 99.16 | 7 |
| ATOM | 15297 | CA | ALA | G | 807 | 18.302 | 93.270 | 97.421 | 1.00 | 100.11 | 6 |
| ATOM | 15298 | CB | ALA | G | 807 | 17.584 | 93.115 | 98.767 | 1.00 | 156.30 | 6 |
| ATOM | 15299 | C | ALA | G | 807 | 17.735 | 92.259 | 96.420 | 1.00 | 100.11 | 6 |
| ATOM | 15300 | O | ALA | G | 807 | 17.049 | 91.310 | 96.814 | 1.00 | 99.81 | 8 |
| ATOM | 15301 | N | THR | G | 808 | 18.028 | 92.453 | 95.137 | 1.00 | 97.48 | 7 |
| ATOM | 15302 | CA | THR | G | 808 | 17.547 | 91.546 | 94.099 | 1.00 | 96.79 | 6 |
| ATOM | 15303 | CB | THR | G | 808 | 18.106 | 90.129 | 94.314 | 1.00 | 67.99 | 6 |
| ATOM | 15304 | OG1 | THR | G | 808 | 19.514 | 90.201 | 94.557 | 1.00 | 68.21 | 8 |
| ATOM | 15305 | CG2 | THR | G | 808 | 17.861 | 89.268 | 93.094 | 1.00 | 68.39 | 6 |
| ATOM | 15306 | C | THR | G | 808 | 16.028 | 91.448 | 94.130 | 1.00 | 97.04 | 6 |
| ATOM | 15307 | O | THR | G | 808 | 15.467 | 90.397 | 93.819 | 1.00 | 97.24 | 8 |
| ATOM | 15308 | N | PRO | G | 809 | 15.338 | 92.540 | 94.500 | 1.00 | 109.80 | 7 |
| ATOM | 15309 | CD | PRO | G | 809 | 15.798 | 93.915 | 94.773 | 1.00 | 123.02 | 6 |
| ATOM | 15310 | CA | PRO | G | 809 | 13.876 | 92.474 | 94.548 | 1.00 | 110.35 | 6 |
| ATOM | 15311 | CB | PRO | G | 809 | 13.473 | 93.944 | 94.638 | 1.00 | 122.89 | 6 |
| ATOM | 15312 | CG | PRO | G | 809 | 14.589 | 94.532 | 95.448 | 1.00 | 122.67 | 6 |
| ATOM | 15313 | C | PRO | G | 809 | 13.243 | 91.761 | 93.359 | 1.00 | 110.55 | 6 |
| ATOM | 15314 | O | PRO | G | 809 | 13.919 | 91.423 | 92.384 | 1.00 | 110.36 | 8 |
| ATOM | 15315 | N | GLU | G | 810 | 11.937 | 91.532 | 93.459 | 1.00 | 61.22 | 7 |
| ATOM | 15316 | CA | GLU | G | 810 | 11.194 | 90.872 | 92.405 | 1.00 | 61.61 | 6 |
| ATOM | 15317 | CB | GLU | G | 810 | 9.689 | 90.968 | 92.665 | 1.00 | 154.34 | 6 |
| ATOM | 15318 | CG | GLU | G | 810 | 8.836 | 90.265 | 91.616 | 1.00 | 156.04 | 6 |
| ATOM | 15319 | CD | GLU | G | 810 | 7.352 | 90.397 | 91.889 | 1.00 | 157.30 | 6 |
| ATOM | 15320 | OE1 | GLU | G | 810 | 6.925 | 90.022 | 93.002 | 1.00 | 157.98 | 8 |
| ATOM | 15321 | OE2 | GLU | G | 810 | 6.616 | 90.869 | 90.995 | 1.00 | 158.08 | 8 |
| ATOM | 15322 | C | GLU | G | 810 | 11.539 | 91.579 | 91.113 | 1.00 | 61.85 | 6 |
| ATOM | 15323 | O | GLU | G | 810 | 11.325 | 91.042 | 90.031 | 1.00 | 61.43 | 8 |
| ATOM | 15324 | N | ALA | G | 811 | 12.077 | 92.790 | 91.246 | 1.00 | 133.90 | 7 |
| ATOM | 15325 | CA | ALA | G | 811 | 12.484 | 93.600 | 90.106 | 1.00 | 134.51 | 6 |
| ATOM | 15326 | CB | ALA | G | 811 | 13.454 | 94.685 | 90.555 | 1.00 | 98.09 | 6 |
| ATOM | 15327 | C | ALA | G | 811 | 13.125 | 92.728 | 89.032 | 1.00 | 134.99 | 6 |
| ATOM | 15328 | O | ALA | G | 811 | 13.016 | 93.026 | 87.845 | 1.00 | 135.02 | 8 |
| ATOM | 15329 | N | ALA | G | 812 | 13.796 | 91.656 | 89.448 | 1.00 | 141.80 | 7 |
| ATOM | 15330 | CA | ALA | G | 812 | 14.418 | 90.738 | 88.501 | 1.00 | 141.70 | 6 |
| ATOM | 15331 | CB | ALA | G | 812 | 14.892 | 89.482 | 89.219 | 1.00 | 13.87 | 6 |
| ATOM | 15332 | C | ALA | G | 812 | 13.348 | 90.382 | 87.475 | 1.00 | 142.44 | 6 |
| ATOM | 15333 | O | ALA | G | 812 | 13.531 | 90.572 | 86.274 | 1.00 | 143.66 | 8 |
| ATOM | 15334 | N | ALA | G | 813 | 12.223 | 89.877 | 87.969 | 1.00 | 135.92 | 7 |
| ATOM | 15335 | CA | ALA | G | 813 | 11.101 | 89.506 | 87.118 | 1.00 | 136.06 | 6 |
| ATOM | 15336 | CB | ALA | G | 813 | 10.391 | 88.280 | 87.687 | 1.00 | 184.90 | 6 |
| ATOM | 15337 | C | ALA | G | 813 | 10.128 | 90.678 | 87.018 | 1.00 | 135.37 | 6 |
| ATOM | 15338 | O | ALA | G | 813 | 9.376 | 90.794 | 86.050 | 1.00 | 135.25 | 8 |
| ATOM | 15339 | N | ALA | G | 814 | 10.134 | 91.537 | 88.033 | 1.00 | 90.29 | 7 |
| ATOM | 15340 | CA | ALA | G | 814 | 9.261 | 92.695 | 88.021 | 1.00 | 89.49 | 6 |
| ATOM | 15341 | CB | ALA | G | 814 | 9.516 | 93.574 | 89.229 | 1.00 | 117.54 | 6 |
| ATOM | 15342 | C | ALA | G | 814 | 9.666 | 93.405 | 86.755 | 1.00 | 88.89 | 6 |
| ATOM | 15343 | O | ALA | G | 814 | 8.824 | 93.874 | 85.994 | 1.00 | 89.06 | 8 |
| ATOM | 15344 | N | ALA | G | 815 | 10.972 | 93.463 | 86.524 | 1.00 | 78.06 | 7 |
| ATOM | 15345 | CA | ALA | G | 815 | 11.489 | 94.095 | 85.321 | 1.00 | 77.59 | 6 |
| ATOM | 15346 | CB | ALA | G | 815 | 13.009 | 94.105 | 85.330 | 1.00 | 121.63 | 6 |
| ATOM | 15347 | C | ALA | G | 815 | 10.977 | 93.266 | 84.155 | 1.00 | 77.33 | 6 |
| ATOM | 15348 | O | ALA | G | 815 | 10.612 | 93.806 | 83.110 | 1.00 | 77.37 | 8 |
| ATOM | 15349 | N | ALA | G | 816 | 10.956 | 91.948 | 84.347 | 1.00 | 111.03 | 7 |
| ATOM | 15350 | CA | ALA | G | 816 | 10.470 | 91.033 | 83.321 | 1.00 | 111.59 | 6 |
| ATOM | 15351 | CB | ALA | G | 816 | 10.541 | 89.591 | 83.813 | 1.00 | 107.03 | 6 |
| ATOM | 15352 | C | ALA | G | 816 | 9.030 | 91.416 | 83.024 | 1.00 | 111.47 | 6 |
| ATOM | 15353 | O | ALA | G | 816 | 8.094 | 90.840 | 83.584 | 1.00 | 111.27 | 8 |
| ATOM | 15354 | N | ALA | G | 817 | 8.871 | 92.401 | 82.145 | 1.00 | 157.33 | 7 |
| ATOM | 15355 | CA | ALA | G | 817 | 7.564 | 92.912 | 81.757 | 1.00 | 157.32 | 6 |
| ATOM | 15356 | CB | ALA | G | 817 | 7.049 | 93.865 | 82.833 | 1.00 | 82.47 | 6 |
| ATOM | 15357 | C | ALA | G | 817 | 7.695 | 93.645 | 80.427 | 1.00 | 157.25 | 6 |
| ATOM | 15358 | O | ALA | G | 817 | 7.117 | 93.244 | 79.417 | 1.00 | 157.22 | 8 |
| ATOM | 15359 | N | ARG | G | 818 | 8.467 | 94.727 | 80.451 | 1.00 | 86.52 | 7 |
| ATOM | 15360 | CA | ARG | G | 818 | 8.724 | 95.564 | 79.283 | 1.00 | 86.74 | 6 |
| ATOM | 15361 | CB | ARG | G | 818 | 7.422 | 96.180 | 78.768 | 1.00 | 118.96 | 6 |
| ATOM | 15362 | CG | ARG | G | 818 | 6.868 | 97.250 | 79.685 | 1.00 | 120.10 | 6 |
| ATOM | 15363 | CD | ARG | G | 818 | 5.406 | 97.536 | 79.427 | 1.00 | 121.14 | 6 |
| ATOM | 15364 | NE | ARG | G | 818 | 4.857 | 98.381 | 80.481 | 1.00 | 122.06 | 7 |
| ATOM | 15365 | CZ | ARG | G | 818 | 3.562 | 98.497 | 80.739 | 1.00 | 122.04 | 6 |
| ATOM | 15366 | NH1 | ARG | G | 818 | 2.683 | 97.820 | 80.016 | 1.00 | 122.91 | 7 |
| ATOM | 15367 | NH2 | ARG | G | 818 | 3.149 | 99.279 | 81.723 | 1.00 | 121.62 | 7 |
| ATOM | 15368 | C | ARG | G | 818 | 9.670 | 96.668 | 79.758 | 1.00 | 86.63 | 6 |
| ATOM | 15369 | O | ARG | G | 818 | 9.510 | 97.204 | 80.858 | 1.00 | 86.80 | 8 |
| ATOM | 15370 | N | GLY | G | 819 | 10.655 | 97.014 | 78.938 | 1.00 | 148.56 | 7 |
| ATOM | 15371 | CA | GLY | G | 819 | 11.603 | 98.032 | 79.349 | 1.00 | 147.45 | 6 |
| ATOM | 15372 | C | GLY | G | 819 | 12.590 | 97.394 | 80.310 | 1.00 | 146.54 | 6 |
| ATOM | 15373 | O | GLY | G | 819 | 12.708 | 96.168 | 80.343 | 1.00 | 146.84 | 8 |
| ATOM | 15374 | N | ALA | G | 820 | 13.288 | 98.206 | 81.098 | 1.00 | 81.04 | 7 |
| ATOM | 15375 | CA | ALA | G | 820 | 14.273 | 97.690 | 82.051 | 1.00 | 79.47 | 6 |
| ATOM | 15376 | CB | ALA | G | 820 | 13.591 | 97.257 | 83.321 | 1.00 | 17.11 | 6 |
| ATOM | 15377 | C | ALA | G | 820 | 15.042 | 96.515 | 81.466 | 1.00 | 78.95 | 6 |
| ATOM | 15378 | O | ALA | G | 820 | 14.952 | 95.398 | 81.979 | 1.00 | 78.58 | 8 |
| ATOM | 15379 | N | ALA | G | 821 | 15.788 | 96.768 | 80.392 | 1.00 | 150.37 | 7 |
| ATOM | 15380 | CA | ALA | G | 821 | 16.566 | 95.723 | 79.735 | 1.00 | 149.29 | 6 |
| ATOM | 15381 | CB | ALA | G | 821 | 16.970 | 96.154 | 78.341 | 1.00 | 55.90 | 6 |
| ATOM | 15382 | C | ALA | G | 821 | 17.804 | 95.375 | 80.530 | 1.00 | 148.41 | 6 |
| ATOM | 15383 | O | ALA | G | 821 | 18.388 | 96.224 | 81.202 | 1.00 | 148.78 | 8 |
| ATOM | 15384 | N | ALA | G | 822 | 18.207 | 94.116 | 80.427 | 1.00 | 68.65 | 7 |
| ATOM | 15385 | CA | ALA | G | 822 | 19.374 | 93.624 | 81.136 | 1.00 | 67.90 | 6 |
| ATOM | 15386 | CB | ALA | G | 822 | 19.549 | 92.125 | 80.858 | 1.00 | 40.68 | 6 |
| ATOM | 15387 | C | ALA | G | 822 | 20.661 | 94.403 | 80.793 | 1.00 | 67.30 | 6 |
| ATOM | 15388 | O | ALA | G | 822 | 21.462 | 93.997 | 79.929 | 1.00 | 66.87 | 8 |
| ATOM | 15389 | N | ALA | G | 823 | 20.849 | 95.523 | 81.490 | 1.00 | 61.01 | 7 |
| ATOM | 15390 | CA | ALA | G | 823 | 22.018 | 96.360 | 81.291 | 1.00 | 60.21 | 6 |
| ATOM | 15391 | CB | ALA | G | 823 | 22.116 | 96.768 | 79.831 | 1.00 | 39.99 | 6 |
| ATOM | 15392 | C | ALA | G | 823 | 21.994 | 97.600 | 82.178 | 1.00 | 60.11 | 6 |
| ATOM | 15393 | O | ALA | G | 823 | 21.244 | 98.537 | 81.911 | 1.00 | 60.12 | 8 |
| ATOM | 15394 | N | ALA | G | 824 | 22.817 | 97.578 | 83.229 | 1.00 | 84.93 | 7 |
| ATOM | 15395 | CA | ALA | G | 824 | 22.979 | 98.674 | 84.190 | 1.00 | 84.56 | 6 |
| ATOM | 15396 | CB | ALA | G | 824 | 22.618 | 100.002 | 83.537 | 1.00 | 18.43 | 6 |
| ATOM | 15397 | C | ALA | G | 824 | 22.254 | 98.533 | 85.540 | 1.00 | 84.27 | 6 |
| ATOM | 15398 | O | ALA | G | 824 | 22.837 | 98.021 | 86.493 | 1.00 | 83.62 | 8 |
| ATOM | 15399 | N | ALA | G | 825 | 21.007 | 99.004 | 85.619 | 1.00 | 81.05 | 7 |
| ATOM | 15400 | CA | ALA | G | 825 | 20.191 | 98.957 | 86.844 | 1.00 | 81.12 | 6 |
| ATOM | 15401 | CB | ALA | G | 825 | 18.902 | 98.205 | 86.575 | 1.00 | 39.65 | 6 |
| ATOM | 15402 | C | ALA | G | 825 | 20.899 | 98.351 | 88.054 | 1.00 | 81.39 | 6 |
| ATOM | 15403 | O | ALA | G | 825 | 21.201 | 97.161 | 88.064 | 1.00 | 82.09 | 8 |
| ATOM | 15404 | N | ALA | G | 826 | 21.131 | 99.174 | 89.078 | 1.00 | 104.37 | 7 |
| ATOM | 15405 | CA | ALA | G | 826 | 21.832 | 98.766 | 90.306 | 1.00 | 104.98 | 6 |
| ATOM | 15406 | CB | ALA | G | 826 | 22.309 | 100.029 | 91.069 | 1.00 | 32.21 | 6 |
| ATOM | 15407 | C | ALA | G | 826 | 21.081 | 97.834 | 91.275 | 1.00 | 105.32 | 6 |
| ATOM | 15408 | O | ALA | G | 826 | 19.953 | 97.413 | 91.016 | 1.00 | 105.35 | 8 |
| ATOM | 15409 | N | ALA | G | 827 | 21.745 | 97.514 | 92.387 | 1.00 | 126.68 | 7 |
| ATOM | 15410 | CA | ALA | G | 827 | 21.203 | 96.661 | 93.450 | 1.00 | 127.15 | 6 |
| ATOM | 15411 | CB | ALA | G | 827 | 20.952 | 95.238 | 92.936 | 1.00 | 28.33 | 6 |
| ATOM | 15412 | C | ALA | G | 827 | 22.205 | 96.635 | 94.605 | 1.00 | 127.62 | 6 |
| ATOM | 15413 | O | ALA | G | 827 | 21.866 | 96.940 | 95.748 | 1.00 | 127.49 | 8 |
| ATOM | 15414 | N | VAL | G | 828 | 23.444 | 96.281 | 94.280 | 1.00 | 96.70 | 7 |
| ATOM | 15415 | CA | VAL | G | 828 | 24.553 | 96.198 | 95.238 | 1.00 | 96.39 | 6 |
| ATOM | 15416 | CB | VAL | G | 828 | 24.369 | 95.006 | 96.237 | 1.00 | 123.09 | 6 |
| ATOM | 15417 | CG1 | VAL | G | 828 | 25.706 | 94.627 | 96.861 | 1.00 | 122.85 | 6 |
| ATOM | 15418 | CG2 | VAL | G | 828 | 23.385 | 95.386 | 97.343 | 1.00 | 122.31 | 6 |
| ATOM | 15419 | C | VAL | G | 828 | 25.828 | 95.976 | 94.417 | 1.00 | 95.34 | 6 |
| ATOM | 15420 | O | VAL | G | 828 | 26.952 | 96.125 | 94.917 | 1.00 | 94.78 | 8 |
| ATOM | 15421 | N | ALA | G | 829 | 25.617 | 95.623 | 93.147 | 1.00 | 146.54 | 7 |
| ATOM | 15422 | CA | ALA | G | 829 | 26.684 | 95.355 | 92.179 | 1.00 | 145.16 | 6 |
| ATOM | 15423 | CB | ALA | G | 829 | 27.205 | 93.921 | 92.351 | 1.00 | 155.29 | 6 |
| ATOM | 15424 | C | ALA | G | 829 | 26.172 | 95.560 | 90.747 | 1.00 | 143.43 | 6 |
| ATOM | 15425 | O | ALA | G | 829 | 26.959 | 95.637 | 89.805 | 1.00 | 143.34 | 8 |
| ATOM | 15426 | N | ALA | G | 830 | 24.849 | 95.630 | 90.603 | 1.00 | 63.04 | 7 |
| ATOM | 15427 | CA | ALA | G | 830 | 24.182 | 95.836 | 89.314 | 1.00 | 60.98 | 6 |
| ATOM | 15428 | CB | ALA | G | 830 | 24.969 | 96.865 | 88.478 | 1.00 | 87.55 | 6 |
| ATOM | 15429 | C | ALA | G | 830 | 23.910 | 94.573 | 88.479 | 1.00 | 59.04 | 6 |
| ATOM | 15430 | O | ALA | G | 830 | 24.208 | 93.451 | 88.905 | 1.00 | 57.54 | 8 |
| ATOM | 15431 | N | GLY | G | 831 | 23.314 | 94.789 | 87.300 | 1.00 | 46.63 | 7 |
| ATOM | 15432 | CA | GLY | G | 831 | 22.996 | 93.722 | 86.361 | 1.00 | 46.34 | 6 |
| ATOM | 15433 | C | GLY | G | 831 | 21.617 | 93.092 | 86.496 | 1.00 | 46.26 | 6 |
| ATOM | 15434 | O | GLY | G | 831 | 21.237 | 92.687 | 87.595 | 1.00 | 47.43 | 8 |
| ATOM | 15435 | N | ARG | G | 832 | 20.857 | 93.008 | 85.399 | 1.00 | 60.75 | 7 |
| ATOM | 15436 | CA | ARG | G | 832 | 19.529 | 92.387 | 85.446 | 1.00 | 59.25 | 6 |
| ATOM | 15437 | CB | ARG | G | 832 | 18.550 | 92.991 | 84.436 | 1.00 | 55.85 | 6 |
| ATOM | 15438 | CG | ARG | G | 832 | 17.227 | 92.215 | 84.437 | 1.00 | 56.28 | 6 |
| ATOM | 15439 | CD | ARG | G | 832 | 16.214 | 92.675 | 83.405 | 1.00 | 58.16 | 6 |
| ATOM | 15440 | NE | ARG | G | 832 | 15.203 | 91.637 | 83.192 | 1.00 | 59.37 | 7 |
| ATOM | 15441 | CZ | ARG | G | 832 | 14.202 | 91.710 | 82.316 | 1.00 | 59.76 | 6 |
| ATOM | 15442 | NH1 | ARG | G | 832 | 14.044 | 92.785 | 81.548 | 1.00 | 60.31 | 7 |
| ATOM | 15443 | NH2 | ARG | G | 832 | 13.372 | 90.685 | 82.191 | 1.00 | 58.80 | 7 |
| ATOM | 15444 | C | ARG | G | 832 | 19.629 | 90.902 | 85.164 | 1.00 | 58.48 | 6 |
| ATOM | 15445 | O | ARG | G | 832 | 18.679 | 90.151 | 85.385 | 1.00 | 58.45 | 8 |
| ATOM | 15446 | N | ALA | G | 833 | 20.775 | 90.483 | 84.642 | 1.00 | 43.69 | 7 |
| ATOM | 15447 | CA | ALA | G | 833 | 20.994 | 89.077 | 84.371 | 1.00 | 41.44 | 6 |
| ATOM | 15448 | CB | ALA | G | 833 | 22.145 | 88.879 | 83.418 | 1.00 | 44.74 | 6 |
| ATOM | 15449 | C | ALA | G | 833 | 21.308 | 88.453 | 85.717 | 1.00 | 40.98 | 6 |
| ATOM | 15450 | O | ALA | G | 833 | 22.473 | 88.189 | 86.047 | 1.00 | 40.76 | 8 |
| ATOM | 15451 | N | THR | G | 834 | 20.238 | 88.313 | 86.507 | 1.00 | 65.98 | 7 |
| ATOM | 15452 | CA | THR | G | 834 | 20.220 | 87.681 | 87.833 | 1.00 | 64.42 | 6 |
| ATOM | 15453 | CB | THR | G | 834 | 19.588 | 88.563 | 88.955 | 1.00 | 45.26 | 6 |
| ATOM | 15454 | OG1 | THR | G | 834 | 19.784 | 89.957 | 88.690 | 1.00 | 44.58 | 8 |
| ATOM | 15455 | CG2 | THR | G | 834 | 20.233 | 88.244 | 90.275 | 1.00 | 45.99 | 6 |
| ATOM | 15456 | C | THR | G | 834 | 19.193 | 86.610 | 87.488 | 1.00 | 63.85 | 6 |
| ATOM | 15457 | O | THR | G | 834 | 19.089 | 85.564 | 88.138 | 1.00 | 63.49 | 8 |
| ATOM | 15458 | N | SER | G | 835 | 18.425 | 86.936 | 86.443 | 1.00 | 56.29 | 7 |
| ATOM | 15459 | CA | SER | G | 835 | 17.391 | 86.082 | 85.870 | 1.00 | 55.86 | 6 |
| ATOM | 15460 | CB | SER | G | 835 | 16.744 | 86.803 | 84.666 | 1.00 | 38.75 | 6 |
| ATOM | 15461 | OG | SER | G | 835 | 15.703 | 86.062 | 84.030 | 1.00 | 37.23 | 8 |
| ATOM | 15462 | C | SER | G | 835 | 18.174 | 84.848 | 85.430 | 1.00 | 55.42 | 6 |
| ATOM | 15463 | O | SER | G | 835 | 18.355 | 83.918 | 86.212 | 1.00 | 55.90 | 8 |
| ATOM | 15464 | N | VAL | G | 836 | 18.660 | 84.838 | 84.195 | 1.00 | 57.23 | 7 |
| ATOM | 15465 | CA | VAL | G | 836 | 19.446 | 83.702 | 83.749 | 1.00 | 56.28 | 6 |
| ATOM | 15466 | CB | VAL | G | 836 | 19.782 | 83.756 | 82.206 | 1.00 | 16.17 | 6 |
| ATOM | 15467 | CG1 | VAL | G | 836 | 20.072 | 82.329 | 81.704 | 1.00 | 15.03 | 6 |
| ATOM | 15468 | CG2 | VAL | G | 836 | 18.655 | 84.402 | 81.401 | 1.00 | 13.87 | 6 |
| ATOM | 15469 | C | VAL | G | 836 | 20.749 | 83.800 | 84.564 | 1.00 | 58.01 | 6 |
| ATOM | 15470 | O | VAL | G | 836 | 21.628 | 84.592 | 84.244 | 1.00 | 59.23 | 8 |
| ATOM | 15471 | N | GLY | G | 837 | 20.855 | 83.026 | 85.638 | 1.00 | 36.53 | 7 |
| ATOM | 15472 | CA | GLY | G | 837 | 22.060 | 83.057 | 86.450 | 1.00 | 38.40 | 6 |
| ATOM | 15473 | C | GLY | G | 837 | 22.513 | 81.641 | 86.730 | 1.00 | 40.39 | 6 |
| ATOM | 15474 | O | GLY | G | 837 | 22.492 | 81.195 | 87.878 | 1.00 | 39.49 | 8 |
| ATOM | 15475 | N | ALA | G | 838 | 22.932 | 80.957 | 85.661 | 1.00 | 85.86 | 7 |
| ATOM | 15476 | CA | ALA | G | 838 | 23.381 | 79.554 | 85.657 | 1.00 | 88.47 | 6 |
| ATOM | 15477 | CB | ALA | G | 838 | 23.980 | 79.225 | 84.286 | 1.00 | 13.87 | 6 |
| ATOM | 15478 | C | ALA | G | 838 | 24.315 | 79.048 | 86.766 | 1.00 | 91.15 | 6 |
| ATOM | 15479 | O | ALA | G | 838 | 24.182 | 79.431 | 87.929 | 1.00 | 92.10 | 8 |
| ATOM | 15480 | N | ALA | G | 839 | 25.249 | 78.168 | 86.409 | 1.00 | 80.78 | 7 |
| ATOM | 15481 | CA | ALA | G | 839 | 26.157 | 77.606 | 87.403 | 1.00 | 84.32 | 6 |
| ATOM | 15482 | CB | ALA | G | 839 | 25.361 | 76.824 | 88.413 | 1.00 | 81.40 | 6 |
| ATOM | 15483 | C | ALA | G | 839 | 27.252 | 76.711 | 86.824 | 1.00 | 87.11 | 6 |
| ATOM | 15484 | O | ALA | G | 839 | 28.266 | 77.201 | 86.328 | 1.00 | 88.40 | 8 |
| ATOM | 15485 | N | ALA | G | 840 | 27.044 | 75.397 | 86.915 | 1.00 | 114.09 | 7 |
| ATOM | 15486 | CA | ALA | G | 840 | 27.993 | 74.400 | 86.415 | 1.00 | 115.95 | 6 |
| ATOM | 15487 | CB | ALA | G | 840 | 28.102 | 74.501 | 84.878 | 1.00 | 13.87 | 6 |
| ATOM | 15488 | C | ALA | G | 840 | 29.378 | 74.530 | 87.076 | 1.00 | 117.83 | 6 |
| ATOM | 15489 | O | ALA | G | 840 | 29.822 | 75.631 | 87.404 | 1.00 | 118.76 | 8 |
| ATOM | 15490 | N | ALA | G | 841 | 30.056 | 73.401 | 87.275 | 1.00 | 186.87 | 7 |
| ATOM | 15491 | CA | ALA | G | 841 | 31.374 | 73.398 | 87.918 | 1.00 | 187.60 | 6 |
| ATOM | 15492 | CB | ALA | G | 841 | 31.305 | 72.612 | 89.230 | 1.00 | 86.19 | 6 |
| ATOM | 15493 | C | ALA | G | 841 | 32.496 | 72.841 | 87.039 | 1.00 | 188.21 | 6 |
| ATOM | 15494 | O | ALA | G | 841 | 32.418 | 72.885 | 85.811 | 1.00 | 188.43 | 8 |
| ATOM | 15495 | N | ALA | G | 842 | 33.542 | 72.323 | 87.682 | 1.00 | 167.88 | 7 |
| ATOM | 15496 | CA | ALA | G | 842 | 34.689 | 71.753 | 86.978 | 1.00 | 167.94 | 6 |
| ATOM | 15497 | CB | ALA | G | 842 | 35.985 | 72.119 | 87.696 | 1.00 | 82.15 | 6 |
| ATOM | 15498 | C | ALA | G | 842 | 34.575 | 70.237 | 86.853 | 1.00 | 167.91 | 6 |
| ATOM | 15499 | O | ALA | G | 842 | 35.566 | 69.514 | 86.982 | 1.00 | 168.11 | 8 |
| ATOM | 15500 | N | ALA | G | 843 | 33.356 | 69.766 | 86.612 | 1.00 | 121.25 | 7 |
| ATOM | 15501 | CA | ALA | G | 843 | 33.087 | 68.342 | 86.446 | 1.00 | 120.66 | 6 |
| ATOM | 15502 | CB | ALA | G | 843 | 31.972 | 67.907 | 87.393 | 1.00 | 111.74 | 6 |
| ATOM | 15503 | C | ALA | G | 843 | 32.671 | 68.109 | 84.990 | 1.00 | 120.68 | 6 |
| ATOM | 15504 | O | ALA | G | 843 | 33.176 | 67.200 | 84.322 | 1.00 | 120.87 | 8 |
| ATOM | 15505 | N | ALA | G | 844 | 31.756 | 68.951 | 84.510 | 1.00 | 208.87 | 7 |
| ATOM | 15506 | CA | ALA | G | 844 | 31.255 | 68.884 | 83.139 | 1.00 | 208.87 | 6 |
| ATOM | 15507 | CB | ALA | G | 844 | 29.797 | 68.419 | 83.128 | 1.00 | 47.41 | 6 |
| ATOM | 15508 | C | ALA | G | 844 | 31.365 | 70.259 | 82.485 | 1.00 | 208.87 | 6 |
| ATOM | 15509 | O | ALA | G | 844 | 31.110 | 70.407 | 81.289 | 1.00 | 208.87 | 8 |
| ATOM | 15510 | N | ALA | G | 845 | 31.743 | 71.257 | 83.284 | 1.00 | 194.04 | 7 |
| ATOM | 15511 | CA | ALA | G | 845 | 31.896 | 72.637 | 82.820 | 1.00 | 192.47 | 6 |
| ATOM | 15512 | CB | ALA | G | 845 | 32.714 | 72.664 | 81.533 | 1.00 | 131.73 | 6 |
| ATOM | 15513 | C | ALA | G | 845 | 30.532 | 73.299 | 82.601 | 1.00 | 190.88 | 6 |
| ATOM | 15514 | O | ALA | G | 845 | 29.496 | 72.648 | 82.738 | 1.00 | 192.41 | 8 |
| ATOM | 15515 | N | PRO | G | 846 | 30.512 | 74.606 | 82.271 | 1.00 | 50.98 | 7 |
| ATOM | 15516 | CD | PRO | G | 846 | 31.637 | 75.540 | 82.096 | 1.00 | 103.52 | 6 |
| ATOM | 15517 | CA | PRO | G | 846 | 29.238 | 75.297 | 82.046 | 1.00 | 48.41 | 6 |
| ATOM | 15518 | CB | PRO | G | 846 | 29.656 | 76.756 | 81.952 | 1.00 | 102.16 | 6 |
| ATOM | 15519 | CG | PRO | G | 846 | 30.994 | 76.659 | 81.311 | 1.00 | 103.17 | 6 |
| ATOM | 15520 | C | PRO | G | 846 | 28.568 | 74.806 | 80.770 | 1.00 | 46.22 | 6 |
| ATOM | 15521 | O | PRO | G | 846 | 28.186 | 75.603 | 79.909 | 1.00 | 45.30 | 8 |
| ATOM | 15522 | N | ALA | G | 847 | 28.438 | 73.485 | 80.665 | 1.00 | 85.27 | 7 |
| ATOM | 15523 | CA | ALA | G | 847 | 27.828 | 72.823 | 79.517 | 1.00 | 82.84 | 6 |
| ATOM | 15524 | CB | ALA | G | 847 | 28.130 | 71.327 | 79.563 | 1.00 | 13.87 | 6 |
| ATOM | 15525 | C | ALA | G | 847 | 26.323 | 73.048 | 79.496 | 1.00 | 81.47 | 6 |
| ATOM | 15526 | O | ALA | G | 847 | 25.632 | 72.617 | 78.576 | 1.00 | 81.55 | 8 |
| ATOM | 15527 | N | ALA | G | 848 | 25.822 | 73.723 | 80.521 | 1.00 | 47.32 | 7 |
| ATOM | 15528 | CA | ALA | G | 848 | 24.402 | 74.011 | 80.632 | 1.00 | 46.22 | 6 |
| ATOM | 15529 | CB | ALA | G | 848 | 23.932 | 73.760 | 82.057 | 1.00 | 115.44 | 6 |
| ATOM | 15530 | C | ALA | G | 848 | 24.146 | 75.455 | 80.250 | 1.00 | 45.01 | 6 |
| ATOM | 15531 | O | ALA | G | 848 | 23.040 | 75.816 | 79.851 | 1.00 | 44.26 | 8 |
| ATOM | 15532 | N | ALA | G | 849 | 25.181 | 76.278 | 80.391 | 1.00 | 78.01 | 7 |
| ATOM | 15533 | CA | ALA | G | 849 | 25.098 | 77.695 | 80.061 | 1.00 | 76.65 | 6 |
| ATOM | 15534 | CB | ALA | G | 849 | 26.427 | 78.371 | 80.344 | 1.00 | 92.48 | 6 |
| ATOM | 15535 | C | ALA | G | 849 | 24.774 | 77.791 | 78.589 | 1.00 | 75.26 | 6 |
| ATOM | 15536 | O | ALA | G | 849 | 23.811 | 78.440 | 78.184 | 1.00 | 74.63 | 8 |
| ATOM | 15537 | N | LEU | G | 850 | 25.600 | 77.126 | 77.795 | 1.00 | 56.81 | 7 |
| ATOM | 15538 | CA | LEU | G | 850 | 25.427 | 77.097 | 76.360 | 1.00 | 57.05 | 6 |
| ATOM | 15539 | CB | LEU | G | 850 | 26.667 | 76.471 | 75.731 | 1.00 | 59.78 | 6 |
| ATOM | 15540 | CG | LEU | G | 850 | 27.960 | 77.154 | 76.181 | 1.00 | 60.71 | 6 |
| ATOM | 15541 | CD1 | LEU | G | 850 | 29.175 | 76.290 | 75.879 | 1.00 | 60.01 | 6 |
| ATOM | 15542 | CD2 | LEU | G | 850 | 28.059 | 78.495 | 75.484 | 1.00 | 60.96 | 6 |
| ATOM | 15543 | C | LEU | G | 850 | 24.152 | 76.317 | 75.989 | 1.00 | 57.30 | 6 |
| ATOM | 15544 | O | LEU | G | 850 | 23.514 | 76.616 | 74.980 | 1.00 | 59.09 | 8 |
| ATOM | 15545 | N | LEU | G | 851 | 23.793 | 75.313 | 76.793 | 1.00 | 48.82 | 7 |
| ATOM | 15546 | CA | LEU | G | 851 | 22.573 | 74.538 | 76.555 | 1.00 | 48.00 | 6 |
| ATOM | 15547 | CB | LEU | G | 851 | 22.480 | 73.346 | 77.532 | 1.00 | 43.03 | 6 |
| ATOM | 15548 | CG | LEU | G | 851 | 21.160 | 72.812 | 78.156 | 1.00 | 41.25 | 6 |
| ATOM | 15549 | CD1 | LEU | G | 851 | 20.113 | 72.546 | 77.095 | 1.00 | 38.43 | 6 |
| ATOM | 15550 | CD2 | LEU | G | 851 | 21.449 | 71.525 | 78.960 | 1.00 | 39.11 | 6 |
| ATOM | 15551 | C | LEU | G | 851 | 21.436 | 75.514 | 76.806 | 1.00 | 48.73 | 6 |
| ATOM | 15552 | O | LEU | G | 851 | 20.439 | 75.532 | 76.081 | 1.00 | 48.56 | 8 |
| ATOM | 15553 | N | ALA | G | 852 | 21.605 | 76.332 | 77.842 | 1.00 | 76.04 | 7 |
| ATOM | 15554 | CA | ALA | G | 852 | 20.619 | 77.340 | 78.201 | 1.00 | 76.01 | 6 |
| ATOM | 15555 | CB | ALA | G | 852 | 21.040 | 78.050 | 79.476 | 1.00 | 114.18 | 6 |
| ATOM | 15556 | C | ALA | G | 852 | 20.585 | 78.318 | 77.042 | 1.00 | 75.57 | 6 |
| ATOM | 15557 | O | ALA | G | 852 | 19.539 | 78.878 | 76.700 | 1.00 | 76.61 | 8 |
| ATOM | 15558 | N | VAL | G | 853 | 21.754 | 78.502 | 76.443 | 1.00 | 47.51 | 7 |
| ATOM | 15559 | CA | VAL | G | 853 | 21.933 | 79.385 | 75.305 | 1.00 | 46.91 | 6 |
| ATOM | 15560 | CB | VAL | G | 853 | 23.402 | 79.614 | 75.066 | 1.00 | 27.72 | 6 |
| ATOM | 15561 | CG1 | VAL | G | 853 | 23.625 | 80.212 | 73.694 | 1.00 | 27.61 | 6 |
| ATOM | 15562 | CG2 | VAL | G | 853 | 23.925 | 80.503 | 76.145 | 1.00 | 27.86 | 6 |
| ATOM | 15563 | C | VAL | G | 853 | 21.319 | 78.817 | 74.032 | 1.00 | 47.80 | 6 |
| ATOM | 15564 | O | VAL | G | 853 | 20.977 | 79.565 | 73.105 | 1.00 | 47.89 | 8 |
| ATOM | 15565 | N | ALA | G | 854 | 21.212 | 77.492 | 73.980 | 1.00 | 44.35 | 7 |
| ATOM | 15566 | CA | ALA | G | 854 | 20.623 | 76.838 | 72.831 | 1.00 | 44.74 | 6 |
| ATOM | 15567 | CB | ALA | G | 854 | 20.693 | 75.338 | 72.976 | 1.00 | 13.87 | 6 |
| ATOM | 15568 | C | ALA | G | 854 | 19.190 | 77.307 | 72.875 | 1.00 | 45.80 | 6 |
| ATOM | 15569 | O | ALA | G | 854 | 18.928 | 78.487 | 72.636 | 1.00 | 46.51 | 8 |
| ATOM | 15570 | N | HIS | G | 855 | 18.274 | 76.399 | 73.207 | 1.00 | 34.17 | 7 |
| ATOM | 15571 | CA | HIS | G | 855 | 16.848 | 76.715 | 73.296 | 1.00 | 34.09 | 6 |
| ATOM | 15572 | CB | HIS | G | 855 | 16.262 | 76.104 | 74.564 | 1.00 | 73.08 | 6 |
| ATOM | 15573 | CG | HIS | G | 855 | 16.820 | 74.760 | 74.900 | 1.00 | 75.47 | 6 |
| ATOM | 15574 | CD2 | HIS | G | 855 | 17.886 | 74.084 | 74.409 | 1.00 | 76.75 | 6 |
| ATOM | 15575 | ND1 | HIS | G | 855 | 16.277 | 73.956 | 75.876 | 1.00 | 77.24 | 7 |
| ATOM | 15576 | CE1 | HIS | G | 855 | 16.982 | 72.844 | 75.973 | 1.00 | 78.37 | 6 |
| ATOM | 15577 | NE2 | HIS | G | 855 | 17.964 | 72.896 | 75.092 | 1.00 | 78.10 | 7 |
| ATOM | 15578 | C | HIS | G | 855 | 16.655 | 78.231 | 73.325 | 1.00 | 34.10 | 6 |
| ATOM | 15579 | O | HIS | G | 855 | 16.839 | 78.867 | 74.369 | 1.00 | 32.27 | 8 |
| ATOM | 15580 | N | GLY | G | 856 | 16.309 | 78.785 | 72.163 | 1.00 | 57.69 | 7 |
| ATOM | 15581 | CA | GLY | G | 856 | 16.102 | 80.216 | 71.992 | 1.00 | 60.89 | 6 |
| ATOM | 15582 | C | GLY | G | 856 | 16.225 | 81.136 | 73.193 | 1.00 | 62.46 | 6 |
| ATOM | 15583 | O | GLY | G | 856 | 15.353 | 81.975 | 73.412 | 1.00 | 62.26 | 8 |
| ATOM | 15584 | N | LEU | G | 857 | 17.310 | 81.007 | 73.952 | 1.00 | 131.92 | 7 |
| ATOM | 15585 | CA | LEU | G | 857 | 17.525 | 81.826 | 75.141 | 1.00 | 132.97 | 6 |
| ATOM | 15586 | CB | LEU | G | 857 | 17.232 | 81.007 | 76.396 | 1.00 | 54.85 | 6 |
| ATOM | 15587 | CG | LEU | G | 857 | 16.472 | 81.751 | 77.493 | 1.00 | 55.64 | 6 |
| ATOM | 15588 | CD1 | LEU | G | 857 | 16.724 | 83.264 | 77.381 | 1.00 | 55.61 | 6 |
| ATOM | 15589 | CD2 | LEU | G | 857 | 14.984 | 81.437 | 77.365 | 1.00 | 55.37 | 6 |
| ATOM | 15590 | C | LEU | G | 857 | 18.972 | 82.302 | 75.173 | 1.00 | 133.58 | 6 |
| ATOM | 15591 | O | LEU | G | 857 | 19.717 | 82.017 | 74.235 | 1.00 | 134.35 | 8 |
| ATOM | 15592 | N | LEU | G | 858 | 19.368 | 82.999 | 76.245 | 1.00 | 76.53 | 7 |
| ATOM | 15593 | CA | LEU | G | 858 | 20.738 | 83.520 | 76.381 | 1.00 | 76.29 | 6 |
| ATOM | 15594 | CB | LEU | G | 858 | 21.576 | 82.649 | 77.314 | 1.00 | 53.66 | 6 |
| ATOM | 15595 | CG | LEU | G | 858 | 21.318 | 82.796 | 78.812 | 1.00 | 55.24 | 6 |
| ATOM | 15596 | CD1 | LEU | G | 858 | 22.412 | 82.050 | 79.600 | 1.00 | 56.31 | 6 |
| ATOM | 15597 | CD2 | LEU | G | 858 | 21.304 | 84.273 | 79.187 | 1.00 | 54.77 | 6 |
| ATOM | 15598 | C | LEU | G | 858 | 21.387 | 83.550 | 75.014 | 1.00 | 75.57 | 6 |
| ATOM | 15599 | O | LEU | G | 858 | 22.021 | 82.581 | 74.595 | 1.00 | 75.87 | 8 |
| ATOM | 15600 | N | ASP | G | 859 | 21.229 | 84.670 | 74.324 | 1.00 | 64.08 | 7 |
| ATOM | 15601 | CA | ASP | G | 859 | 21.730 | 84.797 | 72.976 | 1.00 | 62.99 | 6 |
| ATOM | 15602 | CB | ASP | G | 859 | 21.503 | 86.205 | 72.462 | 1.00 | 36.94 | 6 |
| ATOM | 15603 | CG | ASP | G | 859 | 21.144 | 86.214 | 71.006 | 1.00 | 37.04 | 6 |
| ATOM | 15604 | OD1 | ASP | G | 859 | 21.487 | 85.235 | 70.326 | 1.00 | 37.03 | 8 |
| ATOM | 15605 | OD2 | ASP | G | 859 | 20.529 | 87.186 | 70.534 | 1.00 | 37.67 | 8 |
| ATOM | 15606 | C | ASP | G | 859 | 23.166 | 84.404 | 72.707 | 1.00 | 63.17 | 6 |
| ATOM | 15607 | O | ASP | G | 859 | 23.502 | 84.108 | 71.561 | 1.00 | 63.59 | 8 |
| ATOM | 15608 | N | LEU | G | 860 | 24.012 | 84.394 | 73.735 | 1.00 | 67.02 | 7 |
| ATOM | 15609 | CA | LEU | G | 860 | 25.418 | 84.030 | 73.547 | 1.00 | 65.71 | 6 |
| ATOM | 15610 | CB | LEU | G | 860 | 25.557 | 82.766 | 72.703 | 1.00 | 17.81 | 6 |
| ATOM | 15611 | CG | LEU | G | 860 | 26.786 | 82.676 | 71.802 | 1.00 | 14.86 | 6 |
| ATOM | 15612 | CD1 | LEU | G | 860 | 27.904 | 82.080 | 72.574 | 1.00 | 13.87 | 6 |
| ATOM | 15613 | CD2 | LEU | G | 860 | 26.486 | 81.822 | 70.591 | 1.00 | 15.21 | 6 |
| ATOM | 15614 | C | LEU | G | 860 | 26.142 | 85.142 | 72.837 | 1.00 | 66.23 | 6 |
| ATOM | 15615 | O | LEU | G | 860 | 27.361 | 85.261 | 72.968 | 1.00 | 67.08 | 8 |
| ATOM | 15616 | N | GLN | G | 861 | 25.396 | 85.939 | 72.064 | 1.00 | 72.96 | 7 |
| ATOM | 15617 | CA | GLN | G | 861 | 25.959 | 87.077 | 71.335 | 1.00 | 71.46 | 6 |
| ATOM | 15618 | CB | GLN | G | 861 | 24.860 | 87.864 | 70.619 | 1.00 | 78.31 | 6 |
| ATOM | 15619 | CG | GLN | G | 861 | 24.539 | 87.318 | 69.231 | 1.00 | 81.15 | 6 |
| ATOM | 15620 | CD | GLN | G | 861 | 23.057 | 87.374 | 68.877 | 1.00 | 83.32 | 6 |
| ATOM | 15621 | OE1 | GLN | G | 861 | 22.379 | 88.375 | 69.117 | 1.00 | 85.90 | 8 |
| ATOM | 15622 | NE2 | GLN | G | 861 | 22.555 | 86.296 | 68.287 | 1.00 | 82.53 | 7 |
| ATOM | 15623 | C | GLN | G | 861 | 26.576 | 87.892 | 72.437 | 1.00 | 69.40 | 6 |
| ATOM | 15624 | O | GLN | G | 861 | 27.116 | 88.972 | 72.219 | 1.00 | 67.89 | 8 |
| ATOM | 15625 | N | ALA | G | 862 | 26.455 | 87.304 | 73.627 | 1.00 | 71.01 | 7 |
| ATOM | 15626 | CA | ALA | G | 862 | 26.963 | 87.783 | 74.894 | 1.00 | 69.91 | 6 |
| ATOM | 15627 | CB | ALA | G | 862 | 28.175 | 88.635 | 74.677 | 1.00 | 147.44 | 6 |
| ATOM | 15628 | C | ALA | G | 862 | 25.901 | 88.531 | 75.654 | 1.00 | 68.60 | 6 |
| ATOM | 15629 | O | ALA | G | 862 | 25.458 | 89.593 | 75.231 | 1.00 | 68.70 | 8 |
| ATOM | 15630 | N | THR | G | 863 | 25.490 | 87.969 | 76.783 | 1.00 | 30.07 | 7 |
| ATOM | 15631 | CA | THR | G | 863 | 24.452 | 88.593 | 77.585 | 1.00 | 29.08 | 6 |
| ATOM | 15632 | CB | THR | G | 863 | 23.209 | 87.705 | 77.677 | 1.00 | 73.20 | 6 |
| ATOM | 15633 | OG1 | THR | G | 863 | 23.590 | 86.423 | 78.198 | 1.00 | 76.57 | 8 |
| ATOM | 15634 | CG2 | THR | G | 863 | 22.535 | 87.562 | 76.301 | 1.00 | 72.02 | 6 |
| ATOM | 15635 | C | THR | G | 863 | 24.913 | 88.887 | 78.995 | 1.00 | 26.54 | 6 |
| ATOM | 15636 | O | THR | G | 863 | 24.191 | 89.512 | 79.775 | 1.00 | 25.80 | 8 |
| ATOM | 15637 | N | VAL | G | 864 | 26.120 | 88.451 | 79.319 | 1.00 | 23.50 | 7 |
| ATOM | 15638 | CA | VAL | G | 864 | 26.650 | 88.674 | 80.654 | 1.00 | 22.07 | 6 |
| ATOM | 15639 | CB | VAL | G | 864 | 26.755 | 90.173 | 81.011 | 1.00 | 22.87 | 6 |
| ATOM | 15640 | CG1 | VAL | G | 864 | 28.005 | 90.414 | 81.839 | 1.00 | 23.36 | 6 |
| ATOM | 15641 | CG2 | VAL | G | 864 | 26.761 | 91.023 | 79.765 | 1.00 | 22.10 | 6 |
| ATOM | 15642 | C | VAL | G | 864 | 25.699 | 88.033 | 81.650 | 1.00 | 22.10 | 6 |
| ATOM | 15643 | O | VAL | G | 864 | 24.829 | 88.683 | 82.212 | 1.00 | 21.15 | 8 |
| ATOM | 15644 | N | THR | G | 865 | 25.864 | 86.737 | 81.843 | 1.00 | 45.08 | 7 |
| ATOM | 15645 | CA | THR | G | 865 | 25.041 | 85.986 | 82.771 | 1.00 | 45.06 | 6 |
| ATOM | 15646 | CB | THR | G | 865 | 24.487 | 84.729 | 82.066 | 1.00 | 128.34 | 6 |
| ATOM | 15647 | OG1 | THR | G | 865 | 23.562 | 85.136 | 81.050 | 1.00 | 131.99 | 8 |
| ATOM | 15648 | CG2 | THR | G | 865 | 23.804 | 83.790 | 83.044 | 1.00 | 129.13 | 6 |
| ATOM | 15649 | C | THR | G | 865 | 26.023 | 85.599 | 83.865 | 1.00 | 43.63 | 6 |
| ATOM | 15650 | O | THR | G | 865 | 27.160 | 86.088 | 83.877 | 1.00 | 43.54 | 8 |
| ATOM | 15651 | N | THR | G | 866 | 25.593 | 84.776 | 84.813 | 1.00 | 25.61 | 7 |
| ATOM | 15652 | CA | THR | G | 866 | 26.528 | 84.321 | 85.814 | 1.00 | 24.71 | 6 |
| ATOM | 15653 | CB | THR | G | 866 | 25.940 | 84.272 | 87.204 | 1.00 | 31.23 | 6 |
| ATOM | 15654 | OG1 | THR | G | 866 | 25.644 | 85.600 | 87.653 | 1.00 | 31.75 | 8 |
| ATOM | 15655 | CG2 | THR | G | 866 | 26.945 | 83.639 | 88.150 | 1.00 | 31.73 | 6 |
| ATOM | 15656 | C | THR | G | 866 | 26.833 | 82.909 | 85.357 | 1.00 | 25.15 | 6 |
| ATOM | 15657 | O | THR | G | 866 | 25.953 | 82.201 | 84.889 | 1.00 | 24.89 | 8 |
| ATOM | 15658 | N | ALA | G | 867 | 28.090 | 82.518 | 85.447 | 1.00 | 41.03 | 7 |
| ATOM | 15659 | CA | ALA | G | 867 | 28.494 | 81.193 | 85.038 | 1.00 | 41.48 | 6 |
| ATOM | 15660 | CB | ALA | G | 867 | 28.903 | 81.199 | 83.570 | 1.00 | 13.87 | 6 |
| ATOM | 15661 | C | ALA | G | 867 | 29.668 | 80.845 | 85.934 | 1.00 | 42.73 | 6 |
| ATOM | 15662 | O | ALA | G | 867 | 30.497 | 81.700 | 86.229 | 1.00 | 43.69 | 8 |
| ATOM | 15663 | N | ALA | G | 868 | 29.719 | 79.604 | 86.395 | 1.00 | 34.32 | 7 |
| ATOM | 15664 | CA | ALA | G | 868 | 30.795 | 79.174 | 87.265 | 1.00 | 36.10 | 6 |
| ATOM | 15665 | CB | ALA | G | 868 | 30.258 | 78.199 | 88.290 | 1.00 | 42.76 | 6 |
| ATOM | 15666 | C | ALA | G | 868 | 31.920 | 78.534 | 86.460 | 1.00 | 37.63 | 6 |
| ATOM | 15667 | O | ALA | G | 868 | 31.771 | 78.286 | 85.272 | 1.00 | 37.79 | 8 |
| ATOM | 15668 | N | ALA | G | 869 | 33.043 | 78.280 | 87.125 | 1.00 | 66.65 | 7 |
| ATOM | 15669 | CA | ALA | G | 869 | 34.228 | 77.668 | 86.520 | 1.00 | 68.09 | 6 |
| ATOM | 15670 | CB | ALA | G | 869 | 34.647 | 78.440 | 85.274 | 1.00 | 129.92 | 6 |
| ATOM | 15671 | C | ALA | G | 869 | 35.352 | 77.698 | 87.555 | 1.00 | 68.88 | 6 |
| ATOM | 15672 | O | ALA | G | 869 | 36.538 | 77.748 | 87.216 | 1.00 | 68.88 | 8 |
| ATOM | 15673 | N | GLY | G | 870 | 34.951 | 77.657 | 88.821 | 1.00 | 57.15 | 7 |
| ATOM | 15674 | CA | GLY | G | 870 | 35.893 | 77.710 | 89.915 | 1.00 | 58.51 | 6 |
| ATOM | 15675 | C | GLY | G | 870 | 35.780 | 79.091 | 90.528 | 1.00 | 59.51 | 6 |
| ATOM | 15676 | O | GLY | G | 870 | 36.656 | 79.532 | 91.273 | 1.00 | 61.33 | 8 |
| ATOM | 15677 | N | ALA | G | 871 | 34.686 | 79.771 | 90.196 | 1.00 | 56.97 | 7 |
| ATOM | 15678 | CA | ALA | G | 871 | 34.395 | 81.123 | 90.677 | 1.00 | 56.70 | 6 |
| ATOM | 15679 | CB | ALA | G | 871 | 35.563 | 82.068 | 90.378 | 1.00 | 13.87 | 6 |
| ATOM | 15680 | C | ALA | G | 871 | 33.165 | 81.578 | 89.922 | 1.00 | 56.66 | 6 |
| ATOM | 15681 | O | ALA | G | 871 | 32.806 | 80.976 | 88.921 | 1.00 | 57.56 | 8 |
| ATOM | 15682 | N | ARG | G | 872 | 32.511 | 82.630 | 90.385 | 1.00 | 40.49 | 7 |
| ATOM | 15683 | CA | ARG | G | 872 | 31.332 | 83.101 | 89.674 | 1.00 | 39.68 | 6 |
| ATOM | 15684 | CB | ARG | G | 872 | 30.277 | 83.637 | 90.641 | 1.00 | 80.04 | 6 |
| ATOM | 15685 | CG | ARG | G | 872 | 29.585 | 82.579 | 91.465 | 1.00 | 83.64 | 6 |
| ATOM | 15686 | CD | ARG | G | 872 | 28.345 | 83.137 | 92.121 | 1.00 | 85.99 | 6 |
| ATOM | 15687 | NE | ARG | G | 872 | 27.866 | 82.219 | 93.141 | 1.00 | 89.26 | 7 |
| ATOM | 15688 | CZ | ARG | G | 872 | 26.865 | 82.485 | 93.968 | 1.00 | 91.96 | 6 |
| ATOM | 15689 | NH1 | ARG | G | 872 | 26.232 | 83.648 | 93.890 | 1.00 | 93.33 | 7 |
| ATOM | 15690 | NH2 | ARG | G | 872 | 26.513 | 81.598 | 94.889 | 1.00 | 93.53 | 7 |
| ATOM | 15691 | C | ARG | G | 872 | 31.685 | 84.194 | 88.696 | 1.00 | 37.68 | 6 |
| ATOM | 15692 | O | ARG | G | 872 | 32.000 | 85.298 | 89.099 | 1.00 | 36.99 | 8 |
| ATOM | 15693 | N | LEU | G | 873 | 31.621 | 83.902 | 87.409 | 1.00 | 33.92 | 7 |
| ATOM | 15694 | CA | LEU | G | 873 | 31.941 | 84.921 | 86.433 | 1.00 | 33.36 | 6 |
| ATOM | 15695 | CB | LEU | G | 873 | 32.749 | 84.327 | 85.278 | 1.00 | 19.03 | 6 |
| ATOM | 15696 | CG | LEU | G | 873 | 34.138 | 83.757 | 85.548 | 1.00 | 17.72 | 6 |
| ATOM | 15697 | CD1 | LEU | G | 873 | 34.841 | 83.466 | 84.206 | 1.00 | 16.79 | 6 |
| ATOM | 15698 | CD2 | LEU | G | 873 | 34.926 | 84.747 | 86.388 | 1.00 | 16.71 | 6 |
| ATOM | 15699 | C | LEU | G | 873 | 30.680 | 85.576 | 85.882 | 1.00 | 33.95 | 6 |
| ATOM | 15700 | O | LEU | G | 873 | 29.637 | 84.933 | 85.786 | 1.00 | 34.32 | 8 |
| ATOM | 15701 | N | ALA | G | 874 | 30.786 | 86.857 | 85.526 | 1.00 | 44.93 | 7 |
| ATOM | 15702 | CA | ALA | G | 874 | 29.667 | 87.612 | 84.962 | 1.00 | 44.76 | 6 |
| ATOM | 15703 | CB | ALA | G | 874 | 29.794 | 89.074 | 85.328 | 1.00 | 76.23 | 6 |
| ATOM | 15704 | C | ALA | G | 874 | 29.703 | 87.441 | 83.450 | 1.00 | 44.78 | 6 |
| ATOM | 15705 | O | ALA | G | 874 | 28.880 | 87.977 | 82.718 | 1.00 | 45.55 | 8 |
| ATOM | 15706 | N | THR | G | 875 | 30.700 | 86.686 | 83.018 | 1.00 | 14.16 | 7 |
| ATOM | 15707 | CA | THR | G | 875 | 30.958 | 86.334 | 81.632 | 1.00 | 14.84 | 6 |
| ATOM | 15708 | CB | THR | G | 875 | 31.768 | 85.005 | 81.635 | 1.00 | 56.84 | 6 |
| ATOM | 15709 | OG1 | THR | G | 875 | 33.099 | 85.243 | 82.113 | 1.00 | 58.85 | 8 |
| ATOM | 15710 | CG2 | THR | G | 875 | 31.826 | 84.393 | 80.274 | 1.00 | 59.65 | 6 |
| ATOM | 15711 | C | THR | G | 875 | 29.688 | 86.168 | 80.771 | 1.00 | 14.64 | 6 |
| ATOM | 15712 | O | THR | G | 875 | 28.574 | 86.141 | 81.290 | 1.00 | 13.87 | 8 |
| ATOM | 15713 | N | ASN | G | 876 | 29.865 | 86.073 | 79.451 | 1.00 | 36.68 | 7 |
| ATOM | 15714 | CA | ASN | G | 876 | 28.755 | 85.851 | 78.517 | 1.00 | 38.78 | 6 |
| ATOM | 15715 | CB | ASN | G | 876 | 28.829 | 86.815 | 77.347 | 1.00 | 184.15 | 6 |
| ATOM | 15716 | CG | ASN | G | 876 | 29.391 | 88.151 | 77.741 | 1.00 | 190.14 | 6 |
| ATOM | 15717 | OD1 | ASN | G | 876 | 28.968 | 88.748 | 78.730 | 1.00 | 194.99 | 8 |
| ATOM | 15718 | ND2 | ASN | G | 876 | 30.354 | 88.635 | 76.967 | 1.00 | 192.51 | 7 |
| ATOM | 15719 | C | ASN | G | 876 | 28.970 | 84.427 | 78.007 | 1.00 | 38.08 | 6 |
| ATOM | 15720 | O | ASN | G | 876 | 30.101 | 84.030 | 77.758 | 1.00 | 36.14 | 8 |
| ATOM | 15721 | N | PRO | G | 877 | 27.892 | 83.646 | 77.827 | 1.00 | 44.83 | 7 |
| ATOM | 15722 | CD | PRO | G | 877 | 26.502 | 84.063 | 77.581 | 1.00 | 68.44 | 6 |
| ATOM | 15723 | CA | PRO | G | 877 | 28.105 | 82.277 | 77.348 | 1.00 | 44.06 | 6 |
| ATOM | 15724 | CB | PRO | G | 877 | 26.727 | 81.866 | 76.844 | 1.00 | 67.53 | 6 |
| ATOM | 15725 | CG | PRO | G | 877 | 26.107 | 83.154 | 76.453 | 1.00 | 67.57 | 6 |
| ATOM | 15726 | C | PRO | G | 877 | 29.159 | 82.251 | 76.269 | 1.00 | 43.95 | 6 |
| ATOM | 15727 | O | PRO | G | 877 | 29.961 | 81.329 | 76.196 | 1.00 | 44.20 | 8 |
| ATOM | 15728 | N | GLY | G | 878 | 29.165 | 83.295 | 75.449 | 1.00 | 55.73 | 7 |
| ATOM | 15729 | CA | GLY | G | 878 | 30.141 | 83.391 | 74.380 | 1.00 | 55.48 | 6 |
| ATOM | 15730 | C | GLY | G | 878 | 31.567 | 83.342 | 74.890 | 1.00 | 54.89 | 6 |
| ATOM | 15731 | O | GLY | G | 878 | 32.373 | 82.517 | 74.454 | 1.00 | 55.24 | 8 |
| ATOM | 15732 | N | ARG | G | 879 | 31.880 | 84.242 | 75.814 | 1.00 | 31.81 | 7 |
| ATOM | 15733 | CA | ARG | G | 879 | 33.207 | 84.303 | 76.401 | 1.00 | 30.20 | 6 |
| ATOM | 15734 | CB | ARG | G | 879 | 33.220 | 85.275 | 77.565 | 1.00 | 75.79 | 6 |
| ATOM | 15735 | CG | ARG | G | 879 | 32.059 | 86.258 | 77.592 | 1.00 | 77.49 | 6 |
| ATOM | 15736 | CD | ARG | G | 879 | 32.527 | 87.484 | 78.320 | 1.00 | 79.29 | 6 |
| ATOM | 15737 | NE | ARG | G | 879 | 33.866 | 87.775 | 77.830 | 1.00 | 82.26 | 7 |
| ATOM | 15738 | CZ | ARG | G | 879 | 34.149 | 87.970 | 76.548 | 1.00 | 83.44 | 6 |
| ATOM | 15739 | NH1 | ARG | G | 879 | 33.180 | 87.931 | 75.648 | 1.00 | 84.99 | 7 |
| ATOM | 15740 | NH2 | ARG | G | 879 | 35.404 | 88.132 | 76.155 | 1.00 | 83.51 | 7 |
| ATOM | 15741 | C | ARG | G | 879 | 33.507 | 82.914 | 76.919 | 1.00 | 29.15 | 6 |
| ATOM | 15742 | O | ARG | G | 879 | 34.648 | 82.466 | 76.902 | 1.00 | 29.73 | 8 |
| ATOM | 15743 | N | ILE | G | 880 | 32.459 | 82.247 | 77.392 | 1.00 | 38.04 | 7 |
| ATOM | 15744 | CA | ILE | G | 880 | 32.581 | 80.903 | 77.901 | 1.00 | 36.82 | 6 |
| ATOM | 15745 | CB | ILE | G | 880 | 31.246 | 80.398 | 78.486 | 1.00 | 59.31 | 6 |
| ATOM | 15746 | CG2 | ILE | G | 880 | 31.308 | 78.892 | 78.738 | 1.00 | 59.60 | 6 |
| ATOM | 15747 | CG1 | ILE | G | 880 | 30.927 | 81.163 | 79.773 | 1.00 | 60.99 | 6 |
| ATOM | 15748 | CD1 | ILE | G | 880 | 32.017 | 81.068 | 80.842 | 1.00 | 60.31 | 6 |
| ATOM | 15749 | C | ILE | G | 880 | 32.970 | 80.052 | 76.716 | 1.00 | 35.96 | 6 |
| ATOM | 15750 | O | ILE | G | 880 | 33.970 | 79.345 | 76.767 | 1.00 | 37.18 | 8 |
| ATOM | 15751 | N | LEU | G | 881 | 32.191 | 80.135 | 75.639 | 1.00 | 29.30 | 7 |
| ATOM | 15752 | CA | LEU | G | 881 | 32.471 | 79.362 | 74.432 | 1.00 | 26.04 | 6 |
| ATOM | 15753 | CB | LEU | G | 881 | 31.557 | 79.823 | 73.297 | 1.00 | 13.87 | 6 |
| ATOM | 15754 | CG | LEU | G | 881 | 31.811 | 79.291 | 71.890 | 1.00 | 13.87 | 6 |
| ATOM | 15755 | CD1 | LEU | G | 881 | 31.831 | 77.794 | 71.902 | 1.00 | 13.87 | 6 |
| ATOM | 15756 | CD2 | LEU | G | 881 | 30.742 | 79.812 | 70.957 | 1.00 | 13.87 | 6 |
| ATOM | 15757 | C | LEU | G | 881 | 33.943 | 79.504 | 74.034 | 1.00 | 25.98 | 6 |
| ATOM | 15758 | O | LEU | G | 881 | 34.679 | 78.516 | 74.035 | 1.00 | 25.33 | 8 |
| ATOM | 15759 | N | PHE | G | 882 | 34.372 | 80.726 | 73.709 | 1.00 | 36.39 | 7 |
| ATOM | 15760 | CA | PHE | G | 882 | 35.766 | 80.962 | 73.329 | 1.00 | 37.30 | 6 |
| ATOM | 15761 | CB | PHE | G | 882 | 36.038 | 82.463 | 73.082 | 1.00 | 36.31 | 6 |
| ATOM | 15762 | CG | PHE | G | 882 | 37.453 | 82.766 | 72.588 | 1.00 | 35.49 | 6 |
| ATOM | 15763 | CD1 | PHE | G | 882 | 37.754 | 82.769 | 71.230 | 1.00 | 35.22 | 6 |
| ATOM | 15764 | CD2 | PHE | G | 882 | 38.488 | 83.004 | 73.484 | 1.00 | 35.56 | 6 |
| ATOM | 15765 | CE1 | PHE | G | 882 | 39.063 | 83.000 | 70.782 | 1.00 | 35.92 | 6 |
| ATOM | 15766 | CE2 | PHE | G | 882 | 39.787 | 83.233 | 73.039 | 1.00 | 35.40 | 6 |
| ATOM | 15767 | CZ | PHE | G | 882 | 40.071 | 83.229 | 71.689 | 1.00 | 34.89 | 6 |
| ATOM | 15768 | C | PHE | C | 882 | 36.647 | 80.447 | 74.457 | 1.00 | 37.63 | 6 |
| ATOM | 15769 | O | PHE | G | 882 | 37.698 | 79.876 | 74.221 | 1.00 | 38.05 | 8 |
| ATOM | 15770 | N | ALA | G | 883 | 36.201 | 80.638 | 75.688 | 1.00 | 47.63 | 7 |
| ATOM | 15771 | CA | ALA | G | 883 | 36.962 | 80.178 | 76.839 | 1.00 | 48.79 | 6 |
| ATOM | 15772 | CB | ALA | G | 883 | 36.179 | 80.416 | 78.118 | 1.00 | 89.30 | 6 |
| ATOM | 15773 | C | ALA | G | 883 | 37.300 | 78.703 | 76.716 | 1.00 | 50.03 | 6 |
| ATOM | 15774 | O | ALA | G | 883 | 38.377 | 78.276 | 77.131 | 1.00 | 51.77 | 8 |
| ATOM | 15775 | N | ARG | G | 884 | 36.368 | 77.932 | 76.155 | 1.00 | 28.79 | 7 |
| ATOM | 15776 | CA | ARG | G | 884 | 36.531 | 76.486 | 75.953 | 1.00 | 27.87 | 6 |
| ATOM | 15777 | CB | ARG | G | 884 | 35.178 | 75.790 | 75.995 | 1.00 | 47.89 | 6 |
| ATOM | 15778 | CG | ARG | G | 884 | 35.189 | 74.394 | 75.422 | 1.00 | 51.11 | 6 |
| ATOM | 15779 | CD | ARG | G | 884 | 33.772 | 73.872 | 75.289 | 1.00 | 53.02 | 6 |
| ATOM | 15780 | NE | ARG | G | 884 | 33.137 | 73.699 | 76.589 | 1.00 | 55.00 | 7 |
| ATOM | 15781 | CZ | ARG | G | 884 | 31.824 | 73.647 | 76.773 | 1.00 | 55.95 | 6 |
| ATOM | 15782 | NH1 | ARG | G | 884 | 31.006 | 73.757 | 75.735 | 1.00 | 56.59 | 7 |
| ATOM | 15783 | NH2 | ARG | G | 884 | 31.332 | 73.490 | 77.995 | 1.00 | 56.70 | 7 |
| ATOM | 15784 | C | ARG | G | 884 | 37.192 | 76.182 | 74.614 | 1.00 | 26.71 | 6 |
| ATOM | 15785 | O | ARG | G | 884 | 37.993 | 75.254 | 74.511 | 1.00 | 25.97 | 8 |
| ATOM | 15786 | N | ILE | G | 885 | 36.837 | 76.962 | 73.593 | 1.00 | 31.04 | 7 |
| ATOM | 15787 | CA | ILE | G | 885 | 37.407 | 76.790 | 72.262 | 1.00 | 30.99 | 6 |
| ATOM | 15788 | CB | ILE | G | 885 | 37.049 | 77.963 | 71.354 | 1.00 | 43.96 | 6 |
| ATOM | 15789 | CG2 | ILE | G | 885 | 37.462 | 77.648 | 69.928 | 1.00 | 45.49 | 6 |
| ATOM | 15790 | CG1 | ILE | G | 885 | 35.547 | 78.228 | 71.434 | 1.00 | 43.65 | 6 |
| ATOM | 15791 | CD1 | ILE | G | 885 | 35.064 | 79.422 | 70.617 | 1.00 | 43.74 | 6 |
| ATOM | 15792 | C | ILE | G | 885 | 38.922 | 76.719 | 72.388 | 1.00 | 29.83 | 6 |
| ATOM | 15793 | O | ILE | G | 885 | 39.562 | 75.824 | 71.847 | 1.00 | 30.77 | 8 |
| ATOM | 15794 | N | VAL | G | 886 | 39.492 | 77.669 | 73.115 | 1.00 | 27.56 | 7 |
| ATOM | 15795 | CA | VAL | G | 886 | 40.930 | 77.699 | 73.323 | 1.00 | 26.35 | 6 |
| ATOM | 15796 | CB | VAL | G | 886 | 41.389 | 79.069 | 73.895 | 1.00 | 13.87 | 6 |
| ATOM | 15797 | CG1 | VAL | G | 886 | 41.888 | 78.895 | 75.330 | 1.00 | 13.87 | 6 |
| ATOM | 15798 | CG2 | VAL | G | 886 | 42.461 | 79.685 | 73.000 | 1.00 | 13.87 | 6 |
| ATOM | 15799 | C | VAL | G | 886 | 41.275 | 76.601 | 74.327 | 1.00 | 28.11 | 6 |
| ATOM | 15800 | O | VAL | G | 886 | 42.427 | 76.170 | 74.439 | 1.00 | 28.19 | 8 |
| ATOM | 15801 | N | GLY | G | 887 | 40.265 | 76.152 | 75.057 | 1.00 | 24.36 | 7 |
| ATOM | 15802 | CA | GLY | G | 887 | 40.494 | 75.132 | 76.056 | 1.00 | 27.35 | 6 |
| ATOM | 15803 | C | GLY | G | 887 | 40.717 | 73.766 | 75.459 | 1.00 | 29.17 | 6 |
| ATOM | 15804 | O | GLY | G | 887 | 41.823 | 73.231 | 75.496 | 1.00 | 28.82 | 8 |
| ATOM | 15805 | N | GLU | G | 888 | 39.655 | 73.202 | 74.902 | 1.00 | 65.82 | 7 |
| ATOM | 15806 | CA | GLU | G | 888 | 39.720 | 71.881 | 74.313 | 1.00 | 68.64 | 6 |
| ATOM | 15807 | CB | GLU | G | 888 | 38.323 | 71.428 | 73.925 | 1.00 | 39.77 | 6 |
| ATOM | 15808 | CG | GLU | G | 888 | 37.424 | 71.334 | 75.136 | 1.00 | 39.86 | 6 |
| ATOM | 15809 | CD | GLU | G | 888 | 35.985 | 71.159 | 74.769 | 1.00 | 39.84 | 6 |
| ATOM | 15810 | OE1 | GLU | G | 888 | 35.529 | 71.899 | 73.875 | 1.00 | 39.80 | 8 |
| ATOM | 15811 | OE2 | GLU | G | 888 | 35.312 | 70.298 | 75.377 | 1.00 | 40.62 | 8 |
| ATOM | 15812 | C | GLU | G | 888 | 40.647 | 71.844 | 73.127 | 1.00 | 70.30 | 6 |
| ATOM | 15813 | O | GLU | G | 888 | 40.818 | 70.806 | 72.499 | 1.00 | 71.83 | 8 |
| ATOM | 15814 | N | ALA | G | 889 | 41.242 | 72.989 | 72.823 | 1.00 | 43.05 | 7 |
| ATOM | 15815 | CA | ALA | G | 889 | 42.193 | 73.075 | 71.728 | 1.00 | 44.24 | 6 |
| ATOM | 15816 | CB | ALA | G | 889 | 42.284 | 74.501 | 71.220 | 1.00 | 76.02 | 6 |
| ATOM | 15817 | C | ALA | G | 889 | 43.530 | 72.639 | 72.316 | 1.00 | 44.97 | 6 |
| ATOM | 15818 | O | ALA | G | 889 | 44.224 | 71.791 | 71.755 | 1.00 | 43.95 | 8 |
| ATOM | 15819 | N | VAL | G | 890 | 43.874 | 73.227 | 73.458 | 1.00 | 54.40 | 7 |
| ATOM | 15820 | CA | VAL | G | 890 | 45.114 | 72.917 | 74.163 | 1.00 | 57.41 | 6 |
| ATOM | 15821 | CB | VAL | G | 890 | 45.787 | 74.225 | 74.693 | 1.00 | 49.51 | 6 |
| ATOM | 15822 | CG1 | VAL | G | 890 | 47.081 | 73.911 | 75.357 | 1.00 | 48.83 | 6 |
| ATOM | 15823 | CG2 | VAL | G | 890 | 46.047 | 75.188 | 73.560 | 1.00 | 49.11 | 6 |
| ATOM | 15824 | C | VAL | G | 890 | 44.755 | 71.989 | 75.337 | 1.00 | 59.76 | 6 |
| ATOM | 15825 | O | VAL | G | 890 | 43.702 | 71.352 | 75.330 | 1.00 | 61.17 | 8 |
| ATOM | 15826 | N | GLY | G | 891 | 45.624 | 71.904 | 76.336 | 1.00 | 79.51 | 7 |
| ATOM | 15827 | CA | GLY | G | 891 | 45.345 | 71.063 | 77.485 | 1.00 | 81.97 | 6 |
| ATOM | 15828 | C | GLY | G | 891 | 44.323 | 71.710 | 78.406 | 1.00 | 83.30 | 6 |
| ATOM | 15829 | O | GLY | G | 891 | 44.633 | 72.050 | 79.554 | 1.00 | 83.11 | 8 |
| ATOM | 15830 | N | ASP | G | 892 | 43.111 | 71.886 | 77.879 | 1.00 | 121.39 | 7 |
| ATOM | 15831 | CA | ASP | G | 892 | 41.962 | 72.476 | 78.580 | 1.00 | 124.21 | 6 |
| ATOM | 15832 | CB | ASP | G | 892 | 41.308 | 71.428 | 79.498 | 1.00 | 208.87 | 6 |
| ATOM | 15833 | CG | ASP | G | 892 | 42.236 | 70.942 | 80.601 | 1.00 | 208.87 | 6 |
| ATOM | 15834 | OD1 | ASP | G | 892 | 42.697 | 71.775 | 81.413 | 1.00 | 208.87 | 8 |
| ATOM | 15835 | OD2 | ASP | G | 892 | 42.498 | 69.721 | 80.658 | 1.00 | 208.87 | 8 |
| ATOM | 15836 | C | ASP | G | 892 | 42.179 | 73.769 | 79.374 | 1.00 | 124.31 | 6 |
| ATOM | 15837 | O | ASP | G | 892 | 43.309 | 74.218 | 79.566 | 1.00 | 124.47 | 8 |
| ATOM | 15838 | N | GLU | G | 893 | 41.070 | 74.358 | 79.826 | 1.00 | 35.77 | 7 |
| ATOM | 15839 | CA | GLU | G | 893 | 41.095 | 75.584 | 80.606 | 1.00 | 35.68 | 6 |
| ATOM | 15840 | CB | GLU | G | 893 | 39.683 | 75.905 | 81.123 | 1.00 | 115.47 | 6 |
| ATOM | 15841 | CG | GLU | G | 893 | 38.669 | 76.228 | 80.012 | 1.00 | 117.40 | 6 |
| ATOM | 15842 | CD | GLU | G | 893 | 37.233 | 76.432 | 80.516 | 1.00 | 118.53 | 6 |
| ATOM | 15843 | OE1 | GLU | G | 893 | 36.967 | 77.406 | 81.261 | 1.00 | 117.96 | 8 |
| ATOM | 15844 | OE2 | GLU | G | 893 | 36.367 | 75.606 | 80.154 | 1.00 | 119.18 | 8 |
| ATOM | 15845 | C | GLU | G | 893 | 42.086 | 75.460 | 81.768 | 1.00 | 35.36 | 6 |
| ATOM | 15846 | O | GLU | G | 893 | 42.601 | 74.372 | 82.051 | 1.00 | 33.68 | 8 |
| ATOM | 15847 | N | LYS | G | 894 | 42.330 | 76.586 | 82.438 | 1.00 | 94.32 | 7 |
| ATOM | 15848 | CA | LYS | G | 894 | 43.287 | 76.689 | 83.542 | 1.00 | 94.70 | 6 |
| ATOM | 15849 | CB | LYS | G | 894 | 43.321 | 75.413 | 84.397 | 1.00 | 94.12 | 6 |
| ATOM | 15850 | CG | LYS | G | 894 | 42.439 | 75.480 | 85.641 | 1.00 | 96.11 | 6 |
| ATOM | 15851 | CD | LYS | G | 894 | 42.664 | 74.283 | 86.567 | 1.00 | 96.16 | 6 |
| ATOM | 15852 | CE | LYS | G | 894 | 41.797 | 74.372 | 87.826 | 1.00 | 95.52 | 6 |
| ATOM | 15853 | NZ | LYS | G | 894 | 41.985 | 73.206 | 88.740 | 1.00 | 94.50 | 7 |
| ATOM | 15854 | C | LYS | G | 894 | 44.650 | 76.928 | 82.907 | 1.00 | 94.03 | 6 |
| ATOM | 15855 | O | LYS | G | 894 | 45.236 | 78.003 | 83.029 | 1.00 | 94.64 | 8 |
| ATOM | 15856 | N | VAL | G | 895 | 45.147 | 75.915 | 82.215 | 1.00 | 49.20 | 7 |
| ATOM | 15857 | CA | VAL | G | 895 | 46.425 | 76.020 | 81.528 | 1.00 | 47.60 | 6 |
| ATOM | 15858 | CB | VAL | G | 895 | 46.765 | 74.711 | 80.762 | 1.00 | 119.60 | 6 |
| ATOM | 15859 | CG1 | VAL | G | 895 | 48.197 | 74.760 | 80.268 | 1.00 | 120.33 | 6 |
| ATOM | 15860 | CG2 | VAL | G | 895 | 46.531 | 73.493 | 81.649 | 1.00 | 119.78 | 6 |
| ATOM | 15861 | C | VAL | G | 895 | 46.266 | 77.140 | 80.505 | 1.00 | 45.77 | 6 |
| ATOM | 15862 | O | VAL | G | 895 | 47.222 | 77.847 | 80.194 | 1.00 | 44.83 | 8 |
| ATOM | 15863 | N | ALA | G | 896 | 45.038 | 77.277 | 79.999 | 1.00 | 60.71 | 7 |
| ATOM | 15864 | CA | ALA | G | 896 | 44.676 | 78.272 | 78.991 | 1.00 | 60.52 | 6 |
| ATOM | 15865 | CB | ALA | G | 896 | 43.807 | 77.638 | 77.925 | 1.00 | 72.91 | 6 |
| ATOM | 15866 | C | ALA | G | 896 | 43.953 | 79.467 | 79.576 | 1.00 | 60.58 | 6 |
| ATOM | 15867 | O | ALA | G | 896 | 43.515 | 80.353 | 78.848 | 1.00 | 60.79 | 8 |
| ATOM | 15868 | N | GLN | G | 897 | 43.799 | 79.481 | 80.891 | 1.00 | 100.94 | 7 |
| ATOM | 15869 | CA | GLN | G | 897 | 43.144 | 80.606 | 81.531 | 1.00 | 100.51 | 6 |
| ATOM | 15870 | CB | GLN | G | 897 | 42.477 | 80.177 | 82.847 | 1.00 | 86.09 | 6 |
| ATOM | 15871 | CG | GLN | G | 897 | 41.343 | 81.096 | 83.327 | 1.00 | 86.84 | 6 |
| ATOM | 15872 | CD | GLN | G | 897 | 40.153 | 81.174 | 82.357 | 1.00 | 87.05 | 6 |
| ATOM | 15873 | OE1 | GLN | G | 897 | 39.147 | 81.826 | 82.641 | 1.00 | 86.67 | 8 |
| ATOM | 15874 | NE2 | GLN | G | 897 | 40.271 | 80.511 | 81.212 | 1.00 | 87.07 | 7 |
| ATOM | 15875 | C | GLN | G | 897 | 44.260 | 81.607 | 81.784 | 1.00 | 100.40 | 6 |
| ATOM | 15876 | O | GLN | G | 897 | 44.012 | 82.747 | 82.159 | 1.00 | 102.19 | 8 |
| ATOM | 15877 | N | GLU | G | 898 | 45.497 | 81.164 | 81.566 | 1.00 | 75.51 | 7 |
| ATOM | 15878 | CA | GLU | G | 898 | 46.671 | 82.012 | 81.745 | 1.00 | 74.64 | 6 |
| ATOM | 15879 | CB | GLU | G | 898 | 47.918 | 81.165 | 81.971 | 1.00 | 66.30 | 6 |
| ATOM | 15880 | CG | GLU | G | 898 | 48.050 | 80.667 | 83.385 | 1.00 | 67.20 | 6 |
| ATOM | 15881 | CD | GLU | G | 898 | 49.062 | 79.553 | 83.523 | 1.00 | 67.60 | 6 |
| ATOM | 15882 | OE1 | GLU | G | 898 | 50.234 | 79.747 | 83.125 | 1.00 | 67.53 | 8 |
| ATOM | 15883 | OE2 | GLU | G | 898 | 48.675 | 78.480 | 84.039 | 1.00 | 67.31 | 8 |
| ATOM | 15884 | C | GLU | G | 898 | 46.867 | 82.865 | 80.510 | 1.00 | 73.74 | 6 |
| ATOM | 15885 | O | GLU | G | 898 | 47.306 | 84.011 | 80.602 | 1.00 | 74.78 | 8 |
| ATOM | 15886 | N | LEU | G | 899 | 46.545 | 82.294 | 79.352 | 1.00 | 45.15 | 7 |
| ATOM | 15887 | CA | LEU | G | 899 | 46.672 | 83.013 | 78.097 | 1.00 | 42.54 | 6 |
| ATOM | 15888 | CB | LEU | G | 899 | 47.508 | 82.255 | 77.075 | 1.00 | 26.37 | 6 |
| ATOM | 15889 | CG | LEU | G | 899 | 48.356 | 81.084 | 77.514 | 1.00 | 26.06 | 6 |
| ATOM | 15890 | CD1 | LEU | G | 899 | 49.116 | 81.462 | 78.783 | 1.00 | 25.38 | 6 |
| ATOM | 15891 | CD2 | LEU | G | 899 | 47.463 | 79.883 | 77.717 | 1.00 | 26.33 | 6 |
| ATOM | 15892 | C | LEU | G | 899 | 45.325 | 83.248 | 77.478 | 1.00 | 41.69 | 6 |
| ATOM | 15893 | O | LEU | G | 899 | 44.893 | 82.475 | 76.635 | 1.00 | 42.31 | 8 |
| ATOM | 15894 | N | ILE | G | 900 | 44.668 | 84.316 | 77.897 | 1.00 | 22.53 | 7 |
| ATOM | 15895 | CA | ILE | G | 900 | 43.375 | 84.703 | 77.358 | 1.00 | 22.79 | 6 |
| ATOM | 15896 | CB | ILE | G | 900 | 42.281 | 83.627 | 77.609 | 1.00 | 36.61 | 6 |
| ATOM | 15897 | CG2 | ILE | G | 900 | 40.897 | 84.202 | 77.398 | 1.00 | 35.79 | 6 |
| ATOM | 15898 | CG1 | ILE | G | 900 | 42.381 | 82.514 | 76.582 | 1.00 | 37.49 | 6 |
| ATOM | 15899 | CD1 | ILE | G | 900 | 41.455 | 81.390 | 76.897 | 1.00 | 40.24 | 6 |
| ATOM | 15900 | C | ILE | G | 900 | 42.968 | 86.012 | 78.022 | 1.00 | 23.21 | 6 |
| ATOM | 15901 | O | ILE | G | 900 | 43.421 | 86.335 | 79.126 | 1.00 | 22.80 | 8 |
| ATOM | 15902 | N | GLN | G | 901 | 42.134 | 86.774 | 77.325 | 1.00 | 19.87 | 7 |
| ATOM | 15903 | CA | GLN | G | 901 | 41.634 | 88.037 | 77.823 | 1.00 | 21.52 | 6 |
| ATOM | 15904 | CB | GLN | G | 901 | 41.628 | 89.039 | 76.674 | 1.00 | 68.31 | 6 |
| ATOM | 15905 | CG | GLN | G | 901 | 43.028 | 89.319 | 76.126 | 1.00 | 69.31 | 6 |
| ATOM | 15906 | CD | GLN | G | 901 | 43.756 | 90.391 | 76.913 | 1.00 | 69.07 | 6 |
| ATOM | 15907 | OE1 | GLN | G | 901 | 43.625 | 91.581 | 76.623 | 1.00 | 68.55 | 8 |
| ATOM | 15908 | NE2 | GLN | G | 901 | 44.514 | 89.979 | 77.923 | 1.00 | 68.90 | 7 |
| ATOM | 15909 | C | GLN | G | 901 | 40.229 | 87.746 | 78.346 | 1.00 | 22.56 | 6 |
| ATOM | 15910 | O | GLN | G | 901 | 39.302 | 88.552 | 78.234 | 1.00 | 21.99 | 8 |
| ATOM | 15911 | N | MET | G | 902 | 40.115 | 86.556 | 78.925 | 1.00 | 56.90 | 7 |
| ATOM | 15912 | CA | MET | G | 902 | 38.889 | 86.015 | 79.490 | 1.00 | 57.86 | 6 |
| ATOM | 15913 | CB | MET | G | 902 | 39.162 | 85.524 | 80.911 | 1.00 | 74.23 | 6 |
| ATOM | 15914 | CG | MET | G | 902 | 38.006 | 84.777 | 81.551 | 1.00 | 76.01 | 6 |
| ATOM | 15915 | SD | MET | G | 902 | 37.447 | 83.344 | 80.628 | 1.00 | 77.57 | 16 |
| ATOM | 15916 | CE | MET | G | 902 | 35.979 | 84.035 | 79.850 | 1.00 | 78.20 | 6 |
| ATOM | 15917 | C | MET | G | 902 | 37.689 | 86.951 | 79.477 | 1.00 | 57.85 | 6 |
| ATOM | 15918 | O | MET | G | 902 | 36.691 | 86.652 | 78.824 | 1.00 | 57.85 | 8 |
| ATOM | 15919 | N | ASP | G | 903 | 37.789 | 88.074 | 80.190 | 1.00 | 35.08 | 7 |
| ATOM | 15920 | CA | ASP | G | 903 | 36.697 | 89.032 | 80.257 | 1.00 | 35.13 | 6 |
| ATOM | 15921 | CB | ASP | G | 903 | 36.767 | 89.869 | 81.529 | 1.00 | 71.82 | 6 |
| ATOM | 15922 | CG | ASP | G | 903 | 38.069 | 90.597 | 81.668 | 1.00 | 75.46 | 6 |
| ATOM | 15923 | OD1 | ASP | G | 903 | 38.706 | 90.871 | 80.630 | 1.00 | 77.78 | 8 |
| ATOM | 15924 | OD2 | ASP | G | 903 | 38.451 | 90.911 | 82.818 | 1.00 | 77.13 | 8 |
| ATOM | 15925 | C | ASP | G | 903 | 36.678 | 89.930 | 79.039 | 1.00 | 34.20 | 6 |
| ATOM | 15926 | O | ASP | G | 903 | 37.645 | 90.639 | 78.733 | 1.00 | 32.37 | 8 |
| ATOM | 15927 | N | VAL | G | 904 | 35.536 | 89.858 | 78.360 | 1.00 | 162.58 | 7 |
| ATOM | 15928 | CA | VAL | G | 904 | 35.201 | 90.558 | 77.123 | 1.00 | 162.19 | 6 |
| ATOM | 15929 | CB | VAL | G | 904 | 33.790 | 91.172 | 77.211 | 1.00 | 40.65 | 6 |
| ATOM | 15930 | CG1 | VAL | G | 904 | 33.117 | 91.147 | 75.858 | 1.00 | 39.86 | 6 |
| ATOM | 15931 | CG2 | VAL | G | 904 | 32.968 | 90.410 | 78.195 | 1.00 | 39.71 | 6 |
| ATOM | 15932 | C | VAL | G | 904 | 36.141 | 91.589 | 76.537 | 1.00 | 163.22 | 6 |
| ATOM | 15933 | O | VAL | G | 904 | 35.938 | 92.793 | 76.680 | 1.00 | 164.16 | 8 |
| ATOM | 15934 | N | PRO | G | 905 | 37.197 | 91.119 | 75.863 | 1.00 | 60.14 | 7 |
| ATOM | 15935 | CD | PRO | G | 905 | 37.735 | 89.749 | 75.957 | 1.00 | 110.94 | 6 |
| ATOM | 15936 | CA | PRO | G | 905 | 38.165 | 92.005 | 75.235 | 1.00 | 59.02 | 6 |
| ATOM | 15937 | CB | PRO | G | 905 | 39.466 | 91.259 | 75.430 | 1.00 | 109.95 | 6 |
| ATOM | 15938 | CG | PRO | G | 905 | 39.035 | 89.849 | 75.192 | 1.00 | 111.71 | 6 |
| ATOM | 15939 | C | PRO | G | 905 | 37.719 | 92.056 | 73.777 | 1.00 | 57.10 | 6 |
| ATOM | 15940 | O | PRO | G | 905 | 37.794 | 93.095 | 73.135 | 1.00 | 57.59 | 8 |
| ATOM | 15941 | N | GLN | G | 906 | 37.231 | 90.916 | 73.285 | 1.00 | 49.21 | 7 |
| ATOM | 15942 | CA | GLN | G | 906 | 36.724 | 90.757 | 71.917 | 1.00 | 49.85 | 6 |
| ATOM | 15943 | CB | GLN | G | 906 | 36.195 | 92.095 | 71.405 | 1.00 | 53.87 | 6 |
| ATOM | 15944 | CG | GLN | G | 906 | 34.901 | 92.512 | 72.055 | 1.00 | 55.86 | 6 |
| ATOM | 15945 | CD | GLN | G | 906 | 34.943 | 92.399 | 73.564 | 1.00 | 57.00 | 6 |
| ATOM | 15946 | OE1 | GLN | G | 906 | 35.161 | 91.321 | 74.113 | 1.00 | 57.22 | 8 |
| ATOM | 15947 | NE2 | GLN | G | 906 | 34.732 | 93.514 | 74.244 | 1.00 | 57.60 | 7 |
| ATOM | 15948 | C | GLN | G | 906 | 37.669 | 90.131 | 70.879 | 1.00 | 49.11 | 6 |
| ATOM | 15949 | O | GLN | G | 906 | 37.238 | 89.687 | 69.803 | 1.00 | 48.41 | 8 |
| ATOM | 15950 | N | GLU | G | 907 | 38.954 | 90.091 | 71.199 | 1.00 | 79.81 | 7 |
| ATOM | 15951 | CA | GLU | G | 907 | 39.919 | 89.507 | 70.288 | 1.00 | 78.33 | 6 |
| ATOM | 15952 | CB | GLU | G | 907 | 39.525 | 88.057 | 70.013 | 1.00 | 46.27 | 6 |
| ATOM | 15953 | CG | GLU | G | 907 | 39.867 | 87.081 | 71.139 | 1.00 | 46.49 | 6 |
| ATOM | 15954 | CD | GLU | G | 907 | 39.044 | 87.268 | 72.387 | 1.00 | 45.93 | 6 |
| ATOM | 15955 | OE1 | GLU | G | 907 | 37.801 | 87.271 | 72.283 | 1.00 | 44.60 | 8 |
| ATOM | 15956 | OE2 | GLU | G | 907 | 39.649 | 87.394 | 73.474 | 1.00 | 45.94 | 8 |
| ATOM | 15957 | C | GLU | G | 907 | 40.017 | 90.296 | 68.976 | 1.00 | 77.28 | 6 |
| ATOM | 15958 | O | GLU | G | 907 | 39.047 | 90.924 | 68.550 | 1.00 | 78.63 | 8 |
| ATOM | 15959 | N | LYS | G | 908 | 41.194 | 90.253 | 68.353 | 1.00 | 24.13 | 7 |
| ATOM | 15960 | CA | LYS | G | 908 | 41.473 | 90.950 | 67.107 | 1.00 | 22.51 | 6 |
| ATOM | 15961 | CB | LYS | G | 908 | 40.815 | 92.330 | 67.108 | 1.00 | 159.89 | 6 |
| ATOM | 15962 | CG | LYS | G | 908 | 41.446 | 93.325 | 66.150 | 1.00 | 164.86 | 6 |
| ATOM | 15963 | CD | LYS | G | 908 | 42.212 | 94.429 | 66.892 | 1.00 | 167.39 | 6 |
| ATOM | 15964 | CE | LYS | G | 908 | 42.750 | 95.498 | 65.930 | 1.00 | 168.09 | 6 |
| ATOM | 15965 | NZ | LYS | G | 908 | 43.541 | 96.566 | 66.618 | 1.00 | 167.48 | 7 |
| ATOM | 15966 | C | LYS | G | 908 | 42.986 | 91.111 | 66.997 | 1.00 | 20.27 | 6 |
| ATOM | 15967 | O | LYS | G | 908 | 43.610 | 90.720 | 66.009 | 1.00 | 18.93 | 8 |
| ATOM | 15968 | N | ASN | G | 909 | 43.583 | 91.689 | 68.031 | 1.00 | 57.22 | 7 |
| ATOM | 15969 | CA | ASN | G | 909 | 45.022 | 91.923 | 68.046 | 1.00 | 55.65 | 6 |
| ATOM | 15970 | CB | ASN | G | 909 | 45.291 | 93.389 | 68.387 | 1.00 | 48.68 | 6 |
| ATOM | 15971 | CG | ASN | G | 909 | 46.433 | 93.979 | 67.589 | 1.00 | 47.29 | 6 |
| ATOM | 15972 | OD1 | ASN | G | 909 | 47.543 | 93.441 | 67.584 | 1.00 | 46.58 | 8 |
| ATOM | 15973 | ND2 | ASN | G | 909 | 46.168 | 95.100 | 66.911 | 1.00 | 46.82 | 7 |
| ATOM | 15974 | C | ASN | G | 909 | 45.638 | 91.037 | 69.109 | 1.00 | 54.42 | 6 |
| ATOM | 15975 | O | ASN | G | 909 | 46.855 | 90.933 | 69.219 | 1.00 | 54.18 | 8 |
| ATOM | 15976 | N | SER | G | 910 | 44.769 | 90.420 | 69.904 | 1.00 | 63.47 | 7 |
| ATOM | 15977 | CA | SER | G | 910 | 45.175 | 89.543 | 70.990 | 1.00 | 60.11 | 6 |
| ATOM | 15978 | CB | SER | G | 910 | 44.116 | 89.497 | 72.077 | 1.00 | 13.87 | 6 |
| ATOM | 15979 | OG | SER | G | 910 | 42.975 | 88.782 | 71.635 | 1.00 | 13.87 | 8 |
| ATOM | 15980 | C | SER | G | 910 | 45.324 | 88.149 | 70.461 | 1.00 | 58.93 | 6 |
| ATOM | 15981 | O | SER | G | 910 | 46.290 | 87.455 | 70.782 | 1.00 | 60.96 | 8 |
| ATOM | 15982 | N | LEU | G | 911 | 44.336 | 87.728 | 69.674 | 1.00 | 20.64 | 7 |
| ATOM | 15983 | CA | LEU | G | 911 | 44.360 | 86.393 | 69.095 | 1.00 | 16.36 | 6 |
| ATOM | 15984 | CB | LEU | G | 911 | 43.449 | 86.335 | 67.870 | 1.00 | 13.87 | 6 |
| ATOM | 15985 | CG | LEU | G | 911 | 42.081 | 85.679 | 68.034 | 1.00 | 13.87 | 6 |
| ATOM | 15986 | CD1 | LEU | G | 911 | 41.751 | 85.421 | 69.480 | 1.00 | 13.87 | 6 |
| ATOM | 15987 | CD2 | LEU | G | 911 | 41.066 | 86.563 | 67.389 | 1.00 | 13.87 | 6 |
| ATOM | 15988 | C | LEU | G | 911 | 45.806 | 86.174 | 68.725 | 1.00 | 15.35 | 6 |
| ATOM | 15989 | O | LEU | G | 911 | 46.438 | 85.228 | 69.183 | 1.00 | 13.87 | 8 |
| ATOM | 15990 | N | LYS | G | 912 | 46.331 | 87.095 | 67.934 | 1.00 | 15.44 | 7 |
| ATOM | 15991 | CA | LYS | G | 912 | 47.719 | 87.037 | 67.539 | 1.00 | 18.81 | 6 |
| ATOM | 15992 | CB | LYS | G | 912 | 48.117 | 88.362 | 66.885 | 1.00 | 42.46 | 6 |
| ATOM | 15993 | CG | LYS | G | 912 | 49.592 | 88.491 | 66.508 | 1.00 | 43.58 | 6 |
| ATOM | 15994 | CD | LYS | G | 912 | 49.831 | 89.784 | 65.744 | 1.00 | 44.30 | 6 |
| ATOM | 15995 | CE | LYS | G | 912 | 51.225 | 89.858 | 65.159 | 1.00 | 45.82 | 6 |
| ATOM | 15996 | NZ | LYS | G | 912 | 51.267 | 90.850 | 64.043 | 1.00 | 43.71 | 7 |
| ATOM | 15997 | C | LYS | G | 912 | 48.584 | 86.772 | 68.780 | 1.00 | 19.99 | 6 |
| ATOM | 15998 | O | LYS | G | 912 | 49.181 | 85.706 | 68.915 | 1.00 | 20.14 | 8 |
| ATOM | 15999 | N | ASP | G | 913 | 48.637 | 87.723 | 69.702 | 1.00 | 47.75 | 7 |
| ATOM | 16000 | CA | ASP | G | 913 | 49.457 | 87.518 | 70.876 | 1.00 | 48.99 | 6 |
| ATOM | 16001 | CB | ASP | G | 913 | 49.361 | 88.710 | 71.826 | 1.00 | 82.36 | 6 |
| ATOM | 16002 | CG | ASP | G | 913 | 50.480 | 88.712 | 72.854 | 1.00 | 88.19 | 6 |
| ATOM | 16003 | OD1 | ASP | G | 913 | 50.269 | 88.161 | 73.955 | 1.00 | 90.42 | 8 |
| ATOM | 16004 | OD2 | ASP | G | 913 | 51.578 | 89.243 | 72.555 | 1.00 | 89.92 | 8 |
| ATOM | 16005 | C | ASP | G | 913 | 49.130 | 86.217 | 71.599 | 1.00 | 47.74 | 6 |
| ATOM | 16006 | O | ASP | G | 913 | 50.046 | 85.531 | 72.053 | 1.00 | 47.79 | 8 |
| ATOM | 16007 | N | LEU | G | 914 | 47.849 | 85.858 | 71.707 | 1.00 | 49.81 | 7 |
| ATOM | 16008 | CA | LEU | G | 914 | 47.502 | 84.598 | 72.367 | 1.00 | 47.58 | 6 |
| ATOM | 16009 | CB | LEU | G | 914 | 46.026 | 84.242 | 72.190 | 1.00 | 13.87 | 6 |
| ATOM | 16010 | CG | LEU | G | 914 | 45.480 | 83.139 | 73.125 | 1.00 | 13.87 | 6 |
| ATOM | 16011 | CD1 | LEU | G | 914 | 44.332 | 82.460 | 72.432 | 1.00 | 13.87 | 6 |
| ATOM | 16012 | CD2 | LEU | G | 914 | 46.523 | 82.098 | 73.501 | 1.00 | 13.87 | 6 |
| ATOM | 16013 | C | LEU | G | 914 | 48.336 | 83.536 | 71.660 | 1.00 | 47.64 | 6 |
| ATOM | 16014 | O | LEU | G | 914 | 49.135 | 82.835 | 72.293 | 1.00 | 47.19 | 8 |
| ATOM | 16015 | N | VAL | G | 915 | 48.132 | 83.435 | 70.344 | 1.00 | 29.23 | 7 |
| ATOM | 16016 | CA | VAL | G | 915 | 48.850 | 82.496 | 69.507 | 1.00 | 27.15 | 6 |
| ATOM | 16017 | CB | VAL | G | 915 | 48.651 | 82.793 | 68.055 | 1.00 | 13.87 | 6 |
| ATOM | 16018 | CG1 | VAL | G | 915 | 49.385 | 81.791 | 67.222 | 1.00 | 13.87 | 6 |
| ATOM | 16019 | CG2 | VAL | G | 915 | 47.213 | 82.767 | 67.748 | 1.00 | 13.87 | 6 |
| ATOM | 16020 | C | VAL | G | 915 | 50.326 | 82.603 | 69.783 | 1.00 | 29.16 | 6 |
| ATOM | 16021 | O | VAL | G | 915 | 51.032 | 81.602 | 69.737 | 1.00 | 30.18 | 8 |
| ATOM | 16022 | N | TYR | G | 916 | 50.811 | 83.811 | 70.055 | 1.00 | 26.69 | 7 |
| ATOM | 16023 | CA | TYR | G | 916 | 52.223 | 83.950 | 70.347 | 1.00 | 28.19 | 6 |
| ATOM | 16024 | CB | TYR | G | 916 | 52.716 | 85.388 | 70.236 | 1.00 | 19.64 | 6 |
| ATOM | 16025 | CG | TYR | G | 916 | 54.173 | 85.517 | 70.687 | 1.00 | 17.65 | 6 |
| ATOM | 16026 | CD1 | TYR | G | 916 | 55.177 | 85.886 | 69.800 | 1.00 | 15.59 | 6 |
| ATOM | 16027 | CE1 | TYR | G | 916 | 56.507 | 85.923 | 70.211 | 1.00 | 14.46 | 6 |
| ATOM | 16028 | CD2 | TYR | G | 916 | 54.555 | 85.198 | 71.996 | 1.00 | 16.37 | 6 |
| ATOM | 16029 | CE2 | TYR | G | 916 | 55.869 | 85.232 | 72.404 | 1.00 | 14.46 | 6 |
| ATOM | 16030 | CZ | TYR | G | 916 | 56.832 | 85.594 | 71.515 | 1.00 | 14.46 | 6 |
| ATOM | 16031 | OH | TYR | G | 916 | 58.124 | 85.650 | 71.952 | 1.00 | 15.03 | 8 |
| ATOM | 16032 | C | TYR | G | 916 | 52.543 | 83.466 | 71.742 | 1.00 | 30.99 | 6 |
| ATOM | 16033 | O | TYR | G | 916 | 53.214 | 82.451 | 71.901 | 1.00 | 31.41 | 8 |
| ATOM | 16034 | N | GLN | G | 917 | 52.089 | 84.205 | 72.756 | 1.00 | 59.90 | 7 |
| ATOM | 16035 | CA | GLN | G | 917 | 52.397 | 83.849 | 74.138 | 1.00 | 63.11 | 6 |
| ATOM | 16036 | CB | GLN | G | 917 | 51.934 | 84.948 | 75.110 | 1.00 | 81.73 | 6 |
| ATOM | 16037 | CG | GLN | G | 917 | 53.070 | 85.932 | 75.512 | 1.00 | 83.43 | 6 |
| ATOM | 16038 | CD | GLN | G | 917 | 53.127 | 87.239 | 74.685 | 1.00 | 83.94 | 6 |
| ATOM | 16039 | OE1 | GLN | G | 917 | 52.944 | 87.238 | 73.463 | 1.00 | 82.83 | 8 |
| ATOM | 16040 | NE2 | GLN | G | 917 | 53.407 | 88.355 | 75.364 | 1.00 | 82.96 | 7 |
| ATOM | 16041 | C | GLN | G | 917 | 51.874 | 82.491 | 74.555 | 1.00 | 64.25 | 6 |
| ATOM | 16042 | O | GLN | G | 917 | 51.684 | 82.217 | 75.735 | 1.00 | 65.21 | 8 |
| ATOM | 16043 | N | ALA | G | 918 | 51.667 | 81.647 | 73.551 | 1.00 | 59.02 | 7 |
| ATOM | 16044 | CA | ALA | G | 918 | 51.209 | 80.274 | 73.706 | 1.00 | 60.12 | 6 |
| ATOM | 16045 | CB | ALA | G | 918 | 49.903 | 80.063 | 72.959 | 1.00 | 68.85 | 6 |
| ATOM | 16046 | C | ALA | G | 918 | 52.315 | 79.458 | 73.061 | 1.00 | 60.57 | 6 |
| ATOM | 16047 | O | ALA | G | 918 | 52.746 | 78.440 | 73.596 | 1.00 | 61.30 | 8 |
| ATOM | 16048 | N | PHE | G | 919 | 52.767 | 79.939 | 71.903 | 1.00 | 49.26 | 7 |
| ATOM | 16049 | CA | PHE | G | 919 | 53.846 | 79.321 | 71.135 | 1.00 | 47.60 | 6 |
| ATOM | 16050 | CB | PHE | G | 919 | 54.330 | 80.267 | 70.027 | 1.00 | 27.52 | 6 |
| ATOM | 16051 | CG | PHE | G | 919 | 55.359 | 79.657 | 69.101 | 1.00 | 22.68 | 6 |
| ATOM | 16052 | CD1 | PHE | G | 919 | 54.978 | 79.089 | 67.893 | 1.00 | 21.13 | 6 |
| ATOM | 16053 | CD2 | PHE | G | 919 | 56.705 | 79.641 | 69.446 | 1.00 | 19.18 | 6 |
| ATOM | 16054 | CE1 | PHE | G | 919 | 55.916 | 78.523 | 67.060 | 1.00 | 19.37 | 6 |
| ATOM | 16055 | CE2 | PHE | G | 919 | 57.646 | 79.076 | 68.612 | 1.00 | 16.91 | 6 |
| ATOM | 16056 | CZ | PHE | G | 919 | 57.251 | 78.518 | 67.422 | 1.00 | 18.48 | 6 |
| ATOM | 16057 | C | PHE | G | 919 | 54.996 | 79.058 | 72.079 | 1.00 | 47.88 | 6 |
| ATOM | 16058 | O | PHE | G | 919 | 55.787 | 78.149 | 71.863 | 1.00 | 48.05 | 8 |
| ATOM | 16059 | N | LEU | G | 920 | 55.085 | 79.874 | 73.121 | 1.00 | 28.94 | 7 |
| ATOM | 16060 | CA | LEU | G | 920 | 56.131 | 79.731 | 74.112 | 1.00 | 30.43 | 6 |
| ATOM | 16061 | CB | LEU | G | 920 | 56.441 | 81.075 | 74.761 | 1.00 | 38.31 | 6 |
| ATOM | 16062 | CG | LEU | G | 920 | 56.834 | 82.276 | 73.897 | 1.00 | 38.74 | 6 |
| ATOM | 16063 | CD1 | LEU | G | 920 | 58.285 | 82.609 | 74.179 | 1.00 | 37.41 | 6 |
| ATOM | 16064 | CD2 | LEU | G | 920 | 56.610 | 82.005 | 72.417 | 1.00 | 38.21 | 6 |
| ATOM | 16065 | C | LEU | G | 920 | 55.689 | 78.753 | 75.197 | 1.00 | 32.43 | 6 |
| ATOM | 16066 | O | LEU | G | 920 | 56.437 | 77.828 | 75.544 | 1.00 | 33.77 | 8 |
| ATOM | 16067 | N | ARG | G | 921 | 54.473 | 78.939 | 75.720 | 1.00 | 36.83 | 7 |
| ATOM | 16068 | CA | ARG | G | 921 | 53.960 | 78.084 | 76.798 | 1.00 | 37.20 | 6 |
| ATOM | 16069 | CB | ARG | G | 921 | 52.768 | 78.752 | 77.490 | 1.00 | 95.12 | 6 |
| ATOM | 16070 | CG | ARG | G | 921 | 53.079 | 80.115 | 78.083 | 1.00 | 99.99 | 6 |
| ATOM | 16071 | CD | ARG | G | 921 | 52.298 | 80.351 | 79.366 | 1.00 | 103.84 | 6 |
| ATOM | 16072 | NE | ARG | G | 921 | 52.402 | 81.737 | 79.817 | 1.00 | 109.71 | 7 |
| ATOM | 16073 | CZ | ARG | G | 921 | 51.989 | 82.173 | 81.003 | 1.00 | 112.84 | 6 |
| ATOM | 16074 | NH1 | ARG | G | 921 | 51.444 | 81.327 | 81.867 | 1.00 | 114.38 | 7 |
| ATOM | 16075 | NH2 | ARG | G | 921 | 52.118 | 83.456 | 81.326 | 1.00 | 113.91 | 7 |
| ATOM | 16076 | C | ARG | G | 921 | 53.565 | 76.681 | 76.368 | 1.00 | 35.46 | 6 |
| ATOM | 16077 | O | ARG | G | 921 | 53.420 | 75.787 | 77.193 | 1.00 | 35.44 | 8 |
| ATOM | 16078 | N | LEU | G | 922 | 53.397 | 76.494 | 75.073 | 1.00 | 64.30 | 7 |
| ATOM | 16079 | CA | LEU | G | 922 | 53.026 | 75.200 | 74.541 | 1.00 | 63.50 | 6 |
| ATOM | 16080 | CB | LEU | G | 922 | 51.507 | 75.146 | 74.366 | 1.00 | 18.17 | 6 |
| ATOM | 16081 | CG | LEU | G | 922 | 50.712 | 75.602 | 75.585 | 1.00 | 16.20 | 6 |
| ATOM | 16082 | CD1 | LEU | G | 922 | 49.233 | 75.769 | 75.264 | 1.00 | 14.05 | 6 |
| ATOM | 16083 | CD2 | LEU | G | 922 | 50.930 | 74.612 | 76.669 | 1.00 | 14.42 | 6 |
| ATOM | 16084 | C | LEU | G | 922 | 53.744 | 75.045 | 73.196 | 1.00 | 63.72 | 6 |
| ATOM | 16085 | O | LEU | G | 922 | 53.769 | 75.982 | 72.390 | 1.00 | 64.88 | 8 |
| ATOM | 16086 | N | GLY | G | 923 | 54.340 | 73.879 | 72.951 | 1.00 | 33.24 | 7 |
| ATOM | 16087 | CA | GLY | G | 923 | 55.049 | 73.674 | 71.694 | 1.00 | 32.46 | 6 |
| ATOM | 16088 | C | GLY | G | 923 | 54.243 | 73.916 | 70.420 | 1.00 | 31.64 | 6 |
| ATOM | 16089 | O | GLY | G | 923 | 53.027 | 74.105 | 70.455 | 1.00 | 32.09 | 8 |
| ATOM | 16090 | N | MET | G | 924 | 54.934 | 73.911 | 69.285 | 1.00 | 24.29 | 7 |
| ATOM | 16091 | CA | MET | G | 924 | 54.301 | 74.118 | 67.988 | 1.00 | 22.47 | 6 |
| ATOM | 16092 | CB | MET | G | 924 | 55.242 | 73.727 | 66.865 | 1.00 | 31.67 | 6 |
| ATOM | 16093 | CG | MET | G | 924 | 56.426 | 74.602 | 66.683 | 1.00 | 30.54 | 6 |
| ATOM | 16094 | SD | MET | G | 924 | 57.356 | 73.819 | 65.405 | 1.00 | 29.77 | 16 |
| ATOM | 16095 | CE | MET | G | 924 | 58.727 | 73.169 | 66.390 | 1.00 | 30.31 | 6 |
| ATOM | 16096 | C | MET | G | 924 | 53.073 | 73.255 | 67.844 | 1.00 | 23.04 | 6 |
| ATOM | 16097 | O | MET | G | 924 | 51.972 | 73.741 | 67.601 | 1.00 | 23.98 | 8 |
| ATOM | 16098 | N | GLU | G | 925 | 53.293 | 71.956 | 67.955 | 1.00 | 36.57 | 7 |
| ATOM | 16099 | CA | GLU | G | 925 | 52.222 | 71.004 | 67.836 | 1.00 | 37.61 | 6 |
| ATOM | 16100 | CB | GLU | G | 925 | 52.603 | 69.682 | 68.521 | 1.00 | 102.24 | 6 |
| ATOM | 16101 | CG | GLU | G | 925 | 53.701 | 69.789 | 69.583 | 1.00 | 106.64 | 6 |
| ATOM | 16102 | CD | GLU | G | 925 | 55.066 | 69.323 | 69.084 | 1.00 | 109.07 | 6 |
| ATOM | 16103 | OE1 | GLU | G | 925 | 55.571 | 69.890 | 68.093 | 1.00 | 110.33 | 8 |
| ATOM | 16104 | OE2 | GLU | G | 925 | 55.640 | 68.389 | 69.687 | 1.00 | 111.51 | 8 |
| ATOM | 16105 | C | GLU | G | 925 | 50.968 | 71.592 | 68.457 | 1.00 | 37.24 | 6 |
| ATOM | 16106 | O | GLU | G | 925 | 49.992 | 71.816 | 67.761 | 1.00 | 38.11 | 8 |
| ATOM | 16107 | N | LYS | G | 926 | 50.989 | 71.885 | 69.750 | 1.00 | 21.59 | 7 |
| ATOM | 16108 | CA | LYS | G | 926 | 49.794 | 72.419 | 70.363 | 1.00 | 21.44 | 6 |
| ATOM | 16109 | CB | LYS | G | 926 | 49.968 | 72.489 | 71.870 | 1.00 | 36.62 | 6 |
| ATOM | 16110 | CG | LYS | G | 926 | 50.161 | 71.124 | 72.487 | 1.00 | 36.08 | 6 |
| ATOM | 16111 | CD | LYS | G | 926 | 49.704 | 71.075 | 73.945 | 1.00 | 35.26 | 6 |
| ATOM | 16112 | CE | LYS | G | 926 | 48.181 | 71.196 | 74.083 | 1.00 | 35.46 | 6 |
| ATOM | 16113 | NZ | LYS | G | 926 | 47.695 | 70.941 | 75.479 | 1.00 | 33.20 | 7 |
| ATOM | 16114 | C | LYS | G | 926 | 49.323 | 73.765 | 69.810 | 1.00 | 22.00 | 6 |
| ATOM | 16115 | O | LYS | G | 926 | 48.147 | 74.099 | 69.911 | 1.00 | 22.56 | 8 |
| ATOM | 16116 | N | THR | G | 927 | 50.205 | 74.557 | 69.220 | 1.00 | 32.53 | 7 |
| ATOM | 16117 | CA | THR | G | 927 | 49.726 | 75.820 | 68.688 | 1.00 | 32.90 | 6 |
| ATOM | 16118 | CB | THR | G | 927 | 50.859 | 76.839 | 68.548 | 1.00 | 38.47 | 6 |
| ATOM | 16119 | OG1 | THR | G | 927 | 51.168 | 77.369 | 69.840 | 1.00 | 40.65 | 8 |
| ATOM | 16120 | CG2 | THR | G | 927 | 50.442 | 77.990 | 67.665 | 1.00 | 38.50 | 6 |
| ATOM | 16121 | C | THR | G | 927 | 49.013 | 75.589 | 67.359 | 1.00 | 33.69 | 6 |
| ATOM | 16122 | O | THR | G | 927 | 48.276 | 76.449 | 66.882 | 1.00 | 35.63 | 8 |
| ATOM | 16123 | N | ALA | G | 928 | 49.209 | 74.416 | 66.767 | 1.00 | 47.73 | 7 |
| ATOM | 16124 | CA | ALA | G | 928 | 48.528 | 74.091 | 65.514 | 1.00 | 46.88 | 6 |
| ATOM | 16125 | CB | ALA | G | 928 | 49.274 | 73.001 | 64.762 | 1.00 | 46.71 | 6 |
| ATOM | 16126 | C | ALA | G | 928 | 47.109 | 73.622 | 65.837 | 1.00 | 47.00 | 6 |
| ATOM | 16127 | O | ALA | G | 928 | 46.167 | 73.939 | 65.108 | 1.00 | 46.91 | 8 |
| ATOM | 16128 | N | ARG | G | 929 | 46.967 | 72.861 | 66.927 | 1.00 | 30.29 | 7 |
| ATOM | 16129 | CA | ARG | G | 929 | 45.661 | 72.369 | 67.352 | 1.00 | 29.29 | 6 |
| ATOM | 16130 | CB | ARG | G | 929 | 45.733 | 71.723 | 68.738 | 1.00 | 75.51 | 6 |
| ATOM | 16131 | CG | ARG | G | 929 | 46.729 | 70.591 | 68.898 | 1.00 | 80.45 | 6 |
| ATOM | 16132 | CD | ARG | G | 929 | 46.268 | 69.314 | 68.217 | 1.00 | 85.22 | 6 |
| ATOM | 16133 | NE | ARG | G | 929 | 47.126 | 68.179 | 68.564 | 1.00 | 90.86 | 7 |
| ATOM | 16134 | CZ | ARG | G | 929 | 47.014 | 66.956 | 68.043 | 1.00 | 94.45 | 6 |
| ATOM | 16135 | NH1 | ARG | G | 929 | 46.079 | 66.686 | 67.137 | 1.00 | 96.35 | 7 |
| ATOM | 16136 | NH2 | ARG | G | 929 | 47.838 | 65.993 | 68.435 | 1.00 | 96.30 | 7 |
| ATOM | 16137 | C | ARG | G | 929 | 44.817 | 73.625 | 67.444 | 1.00 | 27.83 | 6 |
| ATOM | 16138 | O | ARG | G | 929 | 43.850 | 73.794 | 66.695 | 1.00 | 27.04 | 8 |
| ATOM | 16139 | N | LEU | G | 930 | 45.230 | 74.506 | 68.360 | 1.00 | 37.78 | 7 |
| ATOM | 16140 | CA | LEU | G | 930 | 44.580 | 75.786 | 68.633 | 1.00 | 36.08 | 6 |
| ATOM | 16141 | CB | LEU | G | 930 | 45.357 | 76.551 | 69.705 | 1.00 | 38.85 | 6 |
| ATOM | 16142 | CG | LEU | G | 930 | 45.170 | 78.075 | 69.729 | 1.00 | 39.94 | 6 |
| ATOM | 16143 | CD1 | LEU | G | 930 | 43.713 | 78.447 | 69.969 | 1.00 | 40.31 | 6 |
| ATOM | 16144 | CD2 | LEU | G | 930 | 46.067 | 78.661 | 70.807 | 1.00 | 39.71 | 6 |
| ATOM | 16145 | C | LEU | G | 930 | 44.438 | 76.664 | 67.397 | 1.00 | 34.88 | 6 |
| ATOM | 16146 | O | LEU | G | 930 | 43.421 | 77.348 | 67.234 | 1.00 | 35.31 | 8 |
| ATOM | 16147 | N | LEU | G | 931 | 45.456 | 76.663 | 66.537 | 1.00 | 17.30 | 7 |
| ATOM | 16148 | CA | LEU | G | 931 | 45.386 | 77.462 | 65.323 | 1.00 | 15.74 | 6 |
| ATOM | 16149 | CB | LEU | G | 931 | 46.662 | 77.328 | 64.496 | 1.00 | 19.22 | 6 |
| ATOM | 16150 | CG | LEU | G | 931 | 47.465 | 78.611 | 64.291 | 1.00 | 17.16 | 6 |
| ATOM | 16151 | CD1 | LEU | G | 931 | 46.551 | 79.799 | 63.983 | 1.00 | 17.16 | 6 |
| ATOM | 16152 | CD2 | LEU | G | 931 | 48.249 | 78.881 | 65.543 | 1.00 | 17.18 | 6 |
| ATOM | 16153 | C | LEU | G | 931 | 44.192 | 76.981 | 64.504 | 1.00 | 15.89 | 6 |
| ATOM | 16154 | O | LEU | G | 931 | 43.617 | 77.709 | 63.704 | 1.00 | 15.27 | 8 |
| ATOM | 16155 | N | ASP | G | 932 | 43.810 | 75.738 | 64.709 | 1.00 | 30.07 | 7 |
| ATOM | 16156 | CA | ASP | G | 932 | 42.679 | 75.221 | 63.981 | 1.00 | 30.84 | 6 |
| ATOM | 16157 | CB | ASP | G | 932 | 42.922 | 73.771 | 63.641 | 1.00 | 49.32 | 6 |
| ATOM | 16158 | CG | ASP | G | 932 | 43.517 | 73.623 | 62.288 | 1.00 | 49.58 | 6 |
| ATOM | 16159 | OD1 | ASP | G | 932 | 42.754 | 73.822 | 61.324 | 1.00 | 50.23 | 8 |
| ATOM | 16160 | OD2 | ASP | G | 932 | 44.731 | 73.343 | 62.183 | 1.00 | 49.48 | 8 |
| ATOM | 16161 | C | ASP | G | 932 | 41.415 | 75.384 | 64.784 | 1.00 | 30.57 | 6 |
| ATOM | 16162 | O | ASP | G | 932 | 40.408 | 75.881 | 64.272 | 1.00 | 29.68 | 8 |
| ATOM | 16163 | N | ALA | G | 933 | 41.470 | 74.958 | 66.041 | 1.00 | 16.72 | 7 |
| ATOM | 16164 | CA | ALA | G | 933 | 40.331 | 75.087 | 66.913 | 1.00 | 15.81 | 6 |
| ATOM | 16165 | CB | ALA | G | 933 | 40.772 | 75.071 | 68.354 | 1.00 | 13.87 | 6 |
| ATOM | 16166 | C | ALA | G | 933 | 39.754 | 76.435 | 66.547 | 1.00 | 17.01 | 6 |
| ATOM | 16167 | O | ALA | G | 933 | 38.594 | 76.536 | 66.156 | 1.00 | 18.18 | 8 |
| ATOM | 16168 | N | LEU | G | 934 | 40.580 | 77.473 | 66.630 | 1.00 | 47.76 | 7 |
| ATOM | 16169 | CA | LEU | G | 934 | 40.119 | 78.804 | 66.274 | 1.00 | 48.54 | 6 |
| ATOM | 16170 | CB | LEU | G | 934 | 41.274 | 79.789 | 66.331 | 1.00 | 26.43 | 6 |
| ATOM | 16171 | CG | LEU | G | 934 | 41.685 | 80.069 | 67.769 | 1.00 | 25.46 | 6 |
| ATOM | 16172 | CD1 | LEU | G | 934 | 42.888 | 80.979 | 67.763 | 1.00 | 25.41 | 6 |
| ATOM | 16173 | CD2 | LEU | G | 934 | 40.533 | 80.715 | 68.523 | 1.00 | 24.46 | 6 |
| ATOM | 16174 | C | LEU | G | 934 | 39.478 | 78.801 | 64.887 | 1.00 | 49.38 | 6 |
| ATOM | 16175 | O | LEU | G | 934 | 38.260 | 78.894 | 64.774 | 1.00 | 49.81 | 8 |
| ATOM | 16176 | N | LYS | G | 935 | 40.284 | 78.677 | 63.839 | 1.00 | 39.42 | 7 |
| ATOM | 16177 | CA | LYS | G | 935 | 39.746 | 78.649 | 62.489 | 1.00 | 40.39 | 6 |
| ATOM | 16178 | CB | LYS | G | 935 | 40.587 | 77.751 | 61.576 | 1.00 | 22.21 | 6 |
| ATOM | 16179 | CG | LYS | G | 935 | 40.052 | 77.705 | 60.134 | 1.00 | 20.96 | 6 |
| ATOM | 16180 | CD | LYS | G | 935 | 40.696 | 76.653 | 59.240 | 1.00 | 20.28 | 6 |
| ATOM | 16181 | CE | LYS | G | 935 | 40.313 | 75.241 | 59.641 | 1.00 | 22.50 | 6 |
| ATOM | 16182 | NZ | LYS | G | 935 | 41.007 | 74.250 | 58.779 | 1.00 | 21.34 | 7 |
| ATOM | 16183 | C | LYS | G | 935 | 38.305 | 78.135 | 62.469 | 1.00 | 41.82 | 6 |
| ATOM | 16184 | O | LYS | G | 935 | 37.363 | 78.926 | 62.391 | 1.00 | 43.19 | 8 |
| ATOM | 16185 | N | TYR | G | 936 | 38.135 | 76.816 | 62.555 | 1.00 | 41.92 | 7 |
| ATOM | 16186 | CA | TYR | G | 936 | 36.803 | 76.217 | 62.509 | 1.00 | 41.52 | 6 |
| ATOM | 16187 | CB | TYR | G | 936 | 36.797 | 74.858 | 63.205 | 1.00 | 38.50 | 6 |
| ATOM | 16188 | CG | TYR | G | 936 | 35.470 | 74.093 | 63.158 | 1.00 | 39.63 | 6 |
| ATOM | 16189 | CD1 | TYR | G | 936 | 35.399 | 72.761 | 63.592 | 1.00 | 40.47 | 6 |
| ATOM | 16190 | CE1 | TYR | G | 936 | 34.179 | 72.067 | 63.634 | 1.00 | 41.86 | 6 |
| ATOM | 16191 | CD2 | TYR | G | 936 | 34.282 | 74.708 | 62.756 | 1.00 | 40.46 | 6 |
| ATOM | 16192 | CE2 | TYR | G | 936 | 33.055 | 74.025 | 62.801 | 1.00 | 41.21 | 6 |
| ATOM | 16193 | CZ | TYR | G | 936 | 33.009 | 72.707 | 63.244 | 1.00 | 42.57 | 6 |
| ATOM | 16194 | OH | TYR | G | 936 | 31.795 | 72.046 | 63.337 | 1.00 | 42.34 | 8 |
| ATOM | 16195 | C | TYR | G | 936 | 35.749 | 77.110 | 63.127 | 1.00 | 40.80 | 6 |
| ATOM | 16196 | O | TYR | G | 936 | 34.841 | 77.575 | 62.440 | 1.00 | 40.35 | 8 |
| ATOM | 16197 | N | TYR | G | 937 | 35.869 | 77.355 | 64.422 | 1.00 | 31.70 | 7 |
| ATOM | 16198 | CA | TYR | G | 937 | 34.899 | 78.186 | 65.108 | 1.00 | 32.79 | 6 |
| ATOM | 16199 | CB | TYR | G | 937 | 35.287 | 78.315 | 66.566 | 1.00 | 25.49 | 6 |
| ATOM | 16200 | CG | TYR | G | 937 | 34.947 | 77.070 | 67.309 | 1.00 | 26.11 | 6 |
| ATOM | 16201 | CD1 | TYR | G | 937 | 35.069 | 75.831 | 66.703 | 1.00 | 26.57 | 6 |
| ATOM | 16202 | CE1 | TYR | G | 937 | 34.786 | 74.668 | 67.386 | 1.00 | 26.88 | 6 |
| ATOM | 16203 | CD2 | TYR | G | 937 | 34.527 | 77.118 | 68.617 | 1.00 | 26.56 | 6 |
| ATOM | 16204 | CE2 | TYR | G | 937 | 34.245 | 75.966 | 69.316 | 1.00 | 26.74 | 6 |
| ATOM | 16205 | CZ | TYR | G | 937 | 34.379 | 74.737 | 68.699 | 1.00 | 26.70 | 6 |
| ATOM | 16206 | OH | TYR | G | 937 | 34.132 | 73.577 | 69.417 | 1.00 | 27.27 | 8 |
| ATOM | 16207 | C | TYR | G | 937 | 34.697 | 79.555 | 64.489 | 1.00 | 33.93 | 6 |
| ATOM | 16208 | O | TYR | G | 937 | 33.597 | 79.870 | 64.029 | 1.00 | 33.73 | 8 |
| ATOM | 16209 | N | GLY | G | 938 | 35.747 | 80.369 | 64.473 | 1.00 | 35.73 | 7 |
| ATOM | 16210 | CA | GLY | G | 938 | 35.634 | 81.695 | 63.897 | 1.00 | 35.91 | 6 |
| ATOM | 16211 | C | GLY | G | 938 | 34.857 | 81.619 | 62.606 | 1.00 | 35.32 | 6 |
| ATOM | 16212 | O | GLY | G | 938 | 33.959 | 82.419 | 62.337 | 1.00 | 37.07 | 8 |
| ATOM | 16213 | N | PHE | G | 939 | 35.204 | 80.617 | 61.816 | 1.00 | 29.89 | 7 |
| ATOM | 16214 | CA | PHE | G | 939 | 34.564 | 80.386 | 60.537 | 1.00 | 29.60 | 6 |
| ATOM | 16215 | CB | PHE | G | 939 | 35.262 | 79.245 | 59.827 | 1.00 | 25.78 | 6 |
| ATOM | 16216 | CG | PHE | G | 939 | 36.313 | 79.686 | 58.884 | 1.00 | 22.44 | 6 |
| ATOM | 16217 | CD1 | PHE | G | 939 | 37.627 | 79.330 | 59.087 | 1.00 | 21.66 | 6 |
| ATOM | 16218 | CD2 | PHE | G | 939 | 35.980 | 80.430 | 57.775 | 1.00 | 21.01 | 6 |
| ATOM | 16219 | CE1 | PHE | G | 939 | 38.597 | 79.700 | 58.200 | 1.00 | 22.12 | 6 |
| ATOM | 16220 | CE2 | PHE | G | 939 | 36.940 | 80.805 | 56.884 | 1.00 | 21.55 | 6 |
| ATOM | 16221 | CZ | PHE | G | 939 | 38.255 | 80.441 | 57.091 | 1.00 | 21.92 | 6 |
| ATOM | 16222 | C | PHE | G | 939 | 33.076 | 80.076 | 60.617 | 1.00 | 30.01 | 6 |
| ATOM | 16223 | O | PHE | G | 939 | 32.259 | 80.793 | 60.041 | 1.00 | 29.48 | 8 |
| ATOM | 16224 | N | THR | G | 940 | 32.737 | 78.994 | 61.310 | 1.00 | 44.62 | 7 |
| ATOM | 16225 | CA | THR | G | 940 | 31.350 | 78.581 | 61.458 | 1.00 | 46.40 | 6 |
| ATOM | 16226 | CB | THR | G | 940 | 31.268 | 77.198 | 62.091 | 1.00 | 38.21 | 6 |
| ATOM | 16227 | OG1 | THR | G | 940 | 29.894 | 76.852 | 62.320 | 1.00 | 39.10 | 8 |
| ATOM | 16228 | CG2 | THR | G | 940 | 32.012 | 77.197 | 63.406 | 1.00 | 39.48 | 6 |
| ATOM | 16229 | C | THR | G | 940 | 30.531 | 79.550 | 62.319 | 1.00 | 46.96 | 6 |
| ATOM | 16230 | O | THR | G | 940 | 29.306 | 79.632 | 62.190 | 1.00 | 46.79 | 8 |
| ATOM | 16231 | N | LEU | G | 941 | 31.209 | 80.277 | 63.199 | 1.00 | 57.07 | 7 |
| ATOM | 16232 | CA | LEU | G | 941 | 30.542 | 81.229 | 64.071 | 1.00 | 55.80 | 6 |
| ATOM | 16233 | CB | LEU | G | 941 | 31.481 | 81.684 | 65.169 | 1.00 | 17.59 | 6 |
| ATOM | 16234 | CG | LEU | G | 941 | 31.201 | 81.199 | 66.582 | 1.00 | 14.62 | 6 |
| ATOM | 16235 | CD1 | LEU | G | 941 | 29.720 | 80.889 | 66.690 | 1.00 | 13.87 | 6 |
| ATOM | 16236 | CD2 | LEU | G | 941 | 32.093 | 80.008 | 66.908 | 1.00 | 13.87 | 6 |
| ATOM | 16237 | C | LEU | G | 941 | 30.050 | 82.458 | 63.338 | 1.00 | 56.54 | 6 |
| ATOM | 16238 | O | LEU | G | 941 | 28.921 | 82.897 | 63.539 | 1.00 | 58.13 | 8 |
| ATOM | 16239 | N | SER | G | 942 | 30.912 | 83.031 | 62.507 | 1.00 | 38.22 | 7 |
| ATOM | 16240 | CA | SER | G | 942 | 30.550 | 84.221 | 61.754 | 1.00 | 36.17 | 6 |
| ATOM | 16241 | CB | SER | G | 942 | 31.767 | 84.747 | 60.976 | 1.00 | 15.75 | 6 |
| ATOM | 16242 | OG | SER | G | 942 | 32.160 | 83.849 | 59.956 | 1.00 | 13.87 | 8 |
| ATOM | 16243 | C | SER | G | 942 | 29.395 | 83.936 | 60.795 | 1.00 | 36.01 | 6 |
| ATOM | 16244 | O | SER | G | 942 | 28.696 | 84.851 | 60.371 | 1.00 | 36.77 | 8 |
| ATOM | 16245 | N | THR | G | 943 | 29.188 | 82.663 | 60.467 | 1.00 | 43.57 | 7 |
| ATOM | 16246 | CA | THR | G | 943 | 28.117 | 82.270 | 59.551 | 1.00 | 44.46 | 6 |
| ATOM | 16247 | CB | THR | G | 943 | 28.366 | 80.876 | 58.957 | 1.00 | 64.62 | 6 |
| ATOM | 16248 | OG1 | THR | G | 943 | 29.587 | 80.890 | 58.211 | 1.00 | 68.58 | 8 |
| ATOM | 16249 | CG2 | THR | G | 943 | 27.227 | 80.479 | 58.035 | 1.00 | 64.19 | 6 |
| ATOM | 16250 | C | THR | G | 943 | 26.754 | 82.262 | 60.226 | 1.00 | 43.28 | 6 |
| ATOM | 16251 | O | THR | G | 943 | 25.882 | 83.053 | 59.869 | 1.00 | 43.43 | 8 |
| ATOM | 16252 | N | THR | G | 944 | 26.568 | 81.356 | 61.183 | 1.00 | 35.33 | 7 |
| ATOM | 16253 | CA | THR | G | 944 | 25.310 | 81.262 | 61.907 | 1.00 | 33.37 | 6 |
| ATOM | 16254 | CB | THR | G | 944 | 25.474 | 80.478 | 63.197 | 1.00 | 16.49 | 6 |
| ATOM | 16255 | OG1 | THR | G | 944 | 26.259 | 81.242 | 64.118 | 1.00 | 16.86 | 8 |
| ATOM | 16256 | CG2 | THR | G | 944 | 26.162 | 79.181 | 62.935 | 1.00 | 15.53 | 6 |
| ATOM | 16257 | C | THR | G | 944 | 24.866 | 82.669 | 62.288 | 1.00 | 33.77 | 6 |
| ATOM | 16258 | O | THR | G | 944 | 23.706 | 83.047 | 62.105 | 1.00 | 34.79 | 8 |
| ATOM | 16259 | N | SER | G | 945 | 25.811 | 83.437 | 62.818 | 1.00 | 34.99 | 7 |
| ATOM | 16260 | CA | SER | G | 945 | 25.561 | 84.804 | 63.242 | 1.00 | 35.77 | 6 |
| ATOM | 16261 | CB | SER | G | 945 | 26.860 | 85.411 | 63.765 | 1.00 | 60.62 | 6 |
| ATOM | 16262 | OG | SER | G | 945 | 27.357 | 84.637 | 64.841 | 1.00 | 60.25 | 8 |
| ATOM | 16263 | C | SER | G | 945 | 24.973 | 85.660 | 62.120 | 1.00 | 35.50 | 6 |
| ATOM | 16264 | O | SER | G | 945 | 24.229 | 86.604 | 62.372 | 1.00 | 33.61 | 8 |
| ATOM | 16265 | N | GLY | G | 946 | 25.321 | 85.325 | 60.883 | 1.00 | 47.59 | 7 |
| ATOM | 16266 | CA | GLY | G | 946 | 24.804 | 86.044 | 59.727 | 1.00 | 47.53 | 6 |
| ATOM | 16267 | C | GLY | G | 946 | 24.998 | 87.548 | 59.675 | 1.00 | 47.26 | 6 |
| ATOM | 16268 | O | GLY | G | 946 | 24.105 | 88.305 | 60.060 | 1.00 | 47.50 | 8 |
| ATOM | 16269 | N | ILE | G | 947 | 26.148 | 87.984 | 59.169 | 1.00 | 13.87 | 7 |
| ATOM | 16270 | CA | ILE | G | 947 | 26.434 | 89.403 | 59.086 | 1.00 | 13.87 | 6 |
| ATOM | 16271 | CB | ILE | G | 947 | 27.708 | 89.782 | 59.957 | 1.00 | 16.32 | 6 |
| ATOM | 16272 | CG2 | ILE | G | 947 | 27.835 | 88.882 | 61.166 | 1.00 | 13.93 | 6 |
| ATOM | 16273 | CG1 | ILE | G | 947 | 28.995 | 89.609 | 59.166 | 1.00 | 19.05 | 6 |
| ATOM | 16274 | CD1 | ILE | G | 947 | 29.408 | 90.836 | 58.371 | 1.00 | 22.06 | 6 |
| ATOM | 16275 | C | ILE | G | 947 | 26.649 | 89.771 | 57.628 | 1.00 | 13.87 | 6 |
| ATOM | 16276 | O | ILE | G | 947 | 27.322 | 89.053 | 56.927 | 1.00 | 13.87 | 8 |
| ATOM | 16277 | N | ILE | G | 948 | 26.069 | 90.876 | 57.175 | 1.00 | 18.81 | 7 |
| ATOM | 16278 | CA | ILE | G | 948 | 26.226 | 91.336 | 55.794 | 1.00 | 21.25 | 6 |
| ATOM | 16279 | CB | ILE | G | 948 | 24.972 | 91.133 | 54.955 | 1.00 | 69.85 | 6 |
| ATOM | 16280 | CG2 | ILE | G | 948 | 24.818 | 89.694 | 54.598 | 1.00 | 73.01 | 6 |
| ATOM | 16281 | CG1 | ILE | G | 948 | 23.763 | 91.669 | 55.709 | 1.00 | 73.14 | 6 |
| ATOM | 16282 | CD1 | ILE | G | 948 | 23.483 | 90.985 | 57.045 | 1.00 | 75.70 | 6 |
| ATOM | 16283 | C | ILE | G | 948 | 26.475 | 92.825 | 55.814 | 1.00 | 21.28 | 6 |
| ATOM | 16284 | O | ILE | G | 948 | 26.707 | 93.388 | 56.876 | 1.00 | 21.54 | 8 |
| ATOM | 16285 | N | THR | G | 949 | 26.426 | 93.466 | 54.645 | 1.00 | 24.02 | 7 |
| ATOM | 16286 | CA | THR | G | 949 | 26.633 | 94.915 | 54.535 | 1.00 | 24.53 | 6 |
| ATOM | 16287 | CB | THR | G | 949 | 27.927 | 95.319 | 53.733 | 1.00 | 13.87 | 6 |
| ATOM | 16288 | OG1 | THR | G | 949 | 29.050 | 95.405 | 54.612 | 1.00 | 13.87 | 8 |
| ATOM | 16289 | CG2 | THR | G | 949 | 27.764 | 96.671 | 53.081 | 1.00 | 13.87 | 6 |
| ATOM | 16290 | C | THR | G | 949 | 25.465 | 95.354 | 53.730 | 1.00 | 27.51 | 6 |
| ATOM | 16291 | O | THR | G | 949 | 25.432 | 95.114 | 52.539 | 1.00 | 28.51 | 8 |
| ATOM | 16292 | N | ILE | G | 950 | 24.494 | 95.981 | 54.365 | 1.00 | 53.04 | 7 |
| ATOM | 16293 | CA | ILE | G | 950 | 23.344 | 96.437 | 53.612 | 1.00 | 57.03 | 6 |
| ATOM | 16294 | CB | ILE | G | 950 | 22.033 | 96.133 | 54.368 | 1.00 | 150.33 | 6 |
| ATOM | 16295 | CG2 | ILE | G | 950 | 20.906 | 95.944 | 53.381 | 1.00 | 153.09 | 6 |
| ATOM | 16296 | CG1 | ILE | G | 950 | 22.162 | 94.824 | 55.154 | 1.00 | 151.95 | 6 |
| ATOM | 16297 | CD1 | ILE | G | 950 | 20.959 | 94.510 | 56.037 | 1.00 | 149.50 | 6 |
| ATOM | 16298 | C | ILE | G | 950 | 23.484 | 97.942 | 53.389 | 1.00 | 56.42 | 6 |
| ATOM | 16299 | O | ILE | G | 950 | 24.037 | 98.648 | 54.231 | 1.00 | 57.15 | 8 |
| ATOM | 16300 | N | GLY | G | 951 | 23.004 | 98.423 | 52.247 | 1.00 | 36.84 | 7 |
| ATOM | 16301 | CA | GLY | G | 951 | 23.088 | 99.842 | 51.941 | 1.00 | 38.12 | 6 |
| ATOM | 16302 | C | GLY | G | 951 | 23.001 | 100.685 | 53.194 | 1.00 | 39.04 | 6 |
| ATOM | 16303 | O | GLY | G | 951 | 23.877 | 101.516 | 53.451 | 1.00 | 37.91 | 8 |
| ATOM | 16304 | N | ILE | G | 952 | 21.946 | 100.459 | 53.980 | 1.00 | 40.65 | 7 |
| ATOM | 16305 | CA | ILE | G | 952 | 21.740 | 101.196 | 55.226 | 1.00 | 43.79 | 6 |
| ATOM | 16306 | CB | ILE | G | 952 | 20.234 | 101.313 | 55.632 | 1.00 | 54.20 | 6 |
| ATOM | 16307 | CG2 | ILE | G | 952 | 20.125 | 102.006 | 56.989 | 1.00 | 54.70 | 6 |
| ATOM | 16308 | CG1 | ILE | G | 952 | 19.436 | 102.094 | 54.569 | 1.00 | 54.49 | 6 |
| ATOM | 16309 | CD1 | ILE | G | 952 | 17.998 | 102.431 | 54.970 | 1.00 | 53.89 | 6 |
| ATOM | 16310 | C | ILE | G | 952 | 22.469 | 100.499 | 56.351 | 1.00 | 46.09 | 6 |
| ATOM | 16311 | O | ILE | G | 952 | 23.253 | 101.126 | 57.055 | 1.00 | 47.02 | 8 |
| ATOM | 16312 | N | ASP | G | 953 | 22.196 | 99.205 | 56.522 | 1.00 | 66.77 | 7 |
| ATOM | 16313 | CA | ASP | G | 953 | 22.841 | 98.408 | 57.570 | 1.00 | 68.11 | 6 |
| ATOM | 16314 | CB | ASP | G | 953 | 22.499 | 96.923 | 57.408 | 1.00 | 67.00 | 6 |
| ATOM | 16315 | CG | ASP | G | 953 | 23.227 | 96.033 | 58.410 | 1.00 | 67.44 | 6 |
| ATOM | 16316 | OD1 | ASP | G | 953 | 24.437 | 96.248 | 58.634 | 1.00 | 67.37 | 8 |
| ATOM | 16317 | OD2 | ASP | G | 953 | 22.593 | 95.104 | 58.960 | 1.00 | 66.91 | 8 |
| ATOM | 16318 | C | ASP | G | 953 | 24.343 | 98.604 | 57.440 | 1.00 | 68.64 | 6 |
| ATOM | 16319 | O | ASP | G | 953 | 25.019 | 97.836 | 56.749 | 1.00 | 68.56 | 8 |
| ATOM | 16320 | N | ALA | G | 954 | 24.836 | 99.637 | 58.126 | 1.00 | 38.59 | 7 |
| ATOM | 16321 | CA | ALA | G | 954 | 26.237 | 100.046 | 58.125 | 1.00 | 37.99 | 6 |
| ATOM | 16322 | CB | ALA | G | 954 | 27.152 | 98.880 | 57.761 | 1.00 | 135.82 | 6 |
| ATOM | 16323 | C | ALA | G | 954 | 26.366 | 101.172 | 57.104 | 1.00 | 38.32 | 6 |
| ATOM | 16324 | O | ALA | G | 954 | 25.447 | 101.399 | 56.313 | 1.00 | 36.88 | 8 |
| ATOM | 16325 | N | ALA | G | 955 | 27.511 | 101.852 | 57.105 | 1.00 | 74.92 | 7 |
| ATOM | 16326 | CA | ALA | G | 955 | 27.727 | 102.989 | 56.217 | 1.00 | 77.22 | 6 |
| ATOM | 16327 | CB | ALA | G | 955 | 27.441 | 102.607 | 54.769 | 1.00 | 43.28 | 6 |
| ATOM | 16328 | C | ALA | G | 955 | 26.692 | 103.985 | 56.733 | 1.00 | 78.26 | 6 |
| ATOM | 16329 | O | ALA | G | 955 | 26.122 | 104.786 | 55.992 | 1.00 | 78.73 | 8 |
| ATOM | 16330 | N | VAL | G | 956 | 26.479 | 103.895 | 58.040 | 1.00 | 52.05 | 7 |
| ATOM | 16331 | CA | VAL | G | 956 | 25.515 | 104.681 | 58.789 | 1.00 | 53.27 | 6 |
| ATOM | 16332 | CB | VAL | G | 956 | 25.330 | 104.058 | 60.169 | 1.00 | 42.34 | 6 |
| ATOM | 16333 | CG1 | VAL | G | 956 | 23.988 | 104.458 | 60.745 | 1.00 | 43.43 | 6 |
| ATOM | 16334 | CG2 | VAL | G | 956 | 25.482 | 102.548 | 60.067 | 1.00 | 42.43 | 6 |
| ATOM | 16335 | C | VAL | G | 956 | 25.865 | 106.144 | 58.981 | 1.00 | 54.00 | 6 |
| ATOM | 16336 | O | VAL | G | 956 | 26.384 | 106.528 | 60.026 | 1.00 | 54.46 | 8 |
| ATOM | 16337 | N | ILE | G | 957 | 25.548 | 106.964 | 57.989 | 1.00 | 56.68 | 7 |
| ATOM | 16338 | CA | ILE | G | 957 | 25.838 | 108.390 | 58.056 | 1.00 | 56.95 | 6 |
| ATOM | 16339 | CB | ILE | G | 957 | 25.282 | 109.101 | 56.815 | 1.00 | 58.57 | 6 |
| ATOM | 16340 | CG2 | ILE | G | 957 | 25.523 | 110.599 | 56.928 | 1.00 | 59.02 | 6 |
| ATOM | 16341 | CG1 | ILE | G | 957 | 25.932 | 108.512 | 55.559 | 1.00 | 59.51 | 6 |
| ATOM | 16342 | CD1 | ILE | G | 957 | 25.338 | 108.989 | 54.262 | 1.00 | 60.62 | 6 |
| ATOM | 16343 | C | ILE | G | 957 | 25.268 | 109.057 | 59.311 | 1.00 | 56.81 | 6 |
| ATOM | 16344 | O | ILE | G | 957 | 24.055 | 109.115 | 59.496 | 1.00 | 56.77 | 8 |
| ATOM | 16345 | N | PRO | G | 958 | 26.141 | 109.581 | 60.189 | 1.00 | 47.27 | 7 |
| ATOM | 16346 | CD | PRO | G | 958 | 27.599 | 109.730 | 60.058 | 1.00 | 33.40 | 6 |
| ATOM | 16347 | CA | PRO | G | 958 | 25.676 | 110.235 | 61.412 | 1.00 | 48.03 | 6 |
| ATOM | 16348 | CB | PRO | G | 958 | 26.973 | 110.541 | 62.142 | 1.00 | 33.66 | 6 |
| ATOM | 16349 | CG | PRO | G | 958 | 27.899 | 110.835 | 61.038 | 1.00 | 33.26 | 6 |
| ATOM | 16350 | C | PRO | G | 958 | 24.902 | 111.493 | 61.086 | 1.00 | 49.11 | 6 |
| ATOM | 16351 | O | PRO | G | 958 | 25.359 | 112.318 | 60.301 | 1.00 | 49.29 | 8 |
| ATOM | 16352 | N | GLU | G | 959 | 23.728 | 111.637 | 61.687 | 1.00 | 80.84 | 7 |
| ATOM | 16353 | CA | GLU | G | 959 | 22.896 | 112.809 | 61.449 | 1.00 | 81.89 | 6 |
| ATOM | 16354 | CB | GLU | G | 959 | 21.656 | 112.767 | 62.351 | 1.00 | 181.19 | 6 |
| ATOM | 16355 | CG | GLU | G | 959 | 20.520 | 113.714 | 61.951 | 1.00 | 183.33 | 6 |
| ATOM | 16356 | CD | GLU | G | 959 | 19.508 | 113.074 | 61.008 | 1.00 | 184.49 | 6 |
| ATOM | 16357 | OE1 | GLU | G | 959 | 18.931 | 112.027 | 61.377 | 1.00 | 186.10 | 8 |
| ATOM | 16358 | OE2 | GLU | G | 959 | 19.283 | 113.619 | 59.905 | 1.00 | 183.83 | 8 |
| ATOM | 16359 | C | GLU | G | 959 | 23.740 | 114.035 | 61.780 | 1.00 | 81.69 | 6 |
| ATOM | 16360 | O | GLU | G | 959 | 23.623 | 115.083 | 61.140 | 1.00 | 81.67 | 8 |
| ATOM | 16361 | N | GLU | G | 960 | 24.614 | 113.875 | 62.769 | 1.00 | 48.75 | 7 |
| ATOM | 16362 | CA | GLU | G | 960 | 25.483 | 114.956 | 63.219 | 1.00 | 49.26 | 6 |
| ATOM | 16363 | CB | GLU | G | 960 | 26.228 | 114.532 | 64.484 | 1.00 | 98.69 | 6 |
| ATOM | 16364 | CG | GLU | G | 960 | 27.176 | 113.381 | 64.287 | 1.00 | 99.78 | 6 |
| ATOM | 16365 | CD | GLU | G | 960 | 27.496 | 112.681 | 65.590 | 1.00 | 101.03 | 6 |
| ATOM | 16366 | OE1 | GLU | G | 960 | 26.606 | 111.979 | 66.123 | 1.00 | 101.73 | 8 |
| ATOM | 16367 | OE2 | GLU | G | 960 | 28.633 | 112.837 | 66.085 | 1.00 | 101.81 | 8 |
| ATOM | 16368 | C | GLU | G | 960 | 26.481 | 115.461 | 62.181 | 1.00 | 49.10 | 6 |
| ATOM | 16369 | O | GLU | G | 960 | 27.436 | 116.156 | 62.520 | 1.00 | 47.79 | 8 |
| ATOM | 16370 | N | LYS | G | 961 | 26.268 | 115.111 | 60.918 | 1.00 | 89.18 | 7 |
| ATOM | 16371 | CA | LYS | G | 961 | 27.166 | 115.575 | 59.873 | 1.00 | 89.46 | 6 |
| ATOM | 16372 | CB | LYS | G | 961 | 26.993 | 114.759 | 58.590 | 1.00 | 47.41 | 6 |
| ATOM | 16373 | CG | LYS | G | 961 | 27.998 | 115.100 | 57.494 | 1.00 | 47.64 | 6 |
| ATOM | 16374 | CD | LYS | G | 961 | 27.741 | 114.268 | 56.242 | 1.00 | 48.85 | 6 |
| ATOM | 16375 | CE | LYS | G | 961 | 28.752 | 114.571 | 55.139 | 1.00 | 49.40 | 6 |
| ATOM | 16376 | NZ | LYS | G | 961 | 28.469 | 113.812 | 53.887 | 1.00 | 50.10 | 7 |
| ATOM | 16377 | C | LYS | G | 961 | 26.774 | 117.008 | 59.618 | 1.00 | 89.33 | 6 |
| ATOM | 16378 | O | LYS | G | 961 | 27.611 | 117.904 | 59.671 | 1.00 | 89.78 | 8 |
| ATOM | 16379 | N | GLN | G | 962 | 25.486 | 117.210 | 59.358 | 1.00 | 66.44 | 7 |
| ATOM | 16380 | CA | GLN | G | 962 | 24.965 | 118.539 | 59.092 | 1.00 | 66.75 | 6 |
| ATOM | 16381 | CB | GLN | G | 962 | 23.451 | 118.586 | 59.306 | 1.00 | 95.35 | 6 |
| ATOM | 16382 | CG | GLN | G | 962 | 22.644 | 117.716 | 58.349 | 1.00 | 96.68 | 6 |
| ATOM | 16383 | CD | GLN | G | 962 | 21.161 | 118.076 | 58.341 | 1.00 | 97.30 | 6 |
| ATOM | 16384 | OE1 | GLN | G | 962 | 20.776 | 119.133 | 57.847 | 1.00 | 96.62 | 8 |
| ATOM | 16385 | NE2 | GLN | G | 962 | 20.326 | 117.199 | 58.896 | 1.00 | 97.91 | 7 |
| ATOM | 16386 | C | GLN | G | 962 | 25.655 | 119.536 | 60.016 | 1.00 | 66.49 | 6 |
| ATOM | 16387 | O | GLN | G | 962 | 26.519 | 120.282 | 59.571 | 1.00 | 65.94 | 8 |
| ATOM | 16388 | N | ARG | G | 963 | 25.291 | 119.542 | 61.296 | 1.00 | 71.70 | 7 |
| ATOM | 16389 | CA | ARG | G | 963 | 25.919 | 120.461 | 62.247 | 1.00 | 71.44 | 6 |
| ATOM | 16390 | CB | ARG | G | 963 | 25.975 | 119.837 | 63.659 | 1.00 | 61.60 | 6 |
| ATOM | 16391 | CG | ARG | G | 963 | 27.036 | 120.428 | 64.608 | 1.00 | 60.57 | 6 |
| ATOM | 16392 | CD | ARG | G | 963 | 27.098 | 119.665 | 65.944 | 1.00 | 61.13 | 6 |
| ATOM | 16393 | NE | ARG | G | 963 | 28.468 | 119.499 | 66.441 | 1.00 | 61.92 | 7 |
| ATOM | 16394 | CZ | ARG | G | 963 | 28.787 | 118.963 | 67.621 | 1.00 | 62.68 | 6 |
| ATOM | 16395 | NH1 | ARG | G | 963 | 27.838 | 118.539 | 68.444 | 1.00 | 63.19 | 7 |
| ATOM | 16396 | NH2 | ARG | G | 963 | 30.059 | 118.843 | 67.980 | 1.00 | 61.97 | 7 |
| ATOM | 16397 | C | ARG | G | 963 | 27.322 | 120.806 | 61.752 | 1.00 | 71.22 | 6 |
| ATOM | 16398 | O | ARG | G | 963 | 27.583 | 121.953 | 61.424 | 1.00 | 71.34 | 8 |
| ATOM | 16399 | N | TYR | G | 964 | 28.206 | 119.811 | 61.656 | 1.00 | 107.91 | 7 |
| ATOM | 16400 | CA | TYR | G | 964 | 29.577 | 120.052 | 61.192 | 1.00 | 106.93 | 6 |
| ATOM | 16401 | CB | TYR | G | 964 | 30.375 | 118.751 | 61.153 | 1.00 | 68.73 | 6 |
| ATOM | 16402 | CG | TYR | G | 964 | 30.577 | 118.109 | 62.503 | 1.00 | 68.38 | 6 |
| ATOM | 16403 | CD1 | TYR | G | 964 | 29.520 | 117.488 | 63.157 | 1.00 | 68.40 | 6 |
| ATOM | 16404 | CE1 | TYR | G | 964 | 29.697 | 116.859 | 64.383 | 1.00 | 68.74 | 6 |
| ATOM | 16405 | CD2 | TYR | G | 964 | 31.833 | 118.095 | 63.113 | 1.00 | 68.22 | 6 |
| ATOM | 16406 | CE2 | TYR | G | 964 | 32.027 | 117.468 | 64.342 | 1.00 | 68.53 | 6 |
| ATOM | 16407 | CZ | TYR | G | 964 | 30.952 | 116.849 | 64.971 | 1.00 | 69.16 | 6 |
| ATOM | 16408 | OH | TYR | G | 964 | 31.125 | 116.207 | 66.176 | 1.00 | 69.81 | 8 |
| ATOM | 16409 | C | TYR | G | 964 | 29.600 | 120.706 | 59.814 | 1.00 | 106.42 | 6 |
| ATOM | 16410 | O | TYR | G | 964 | 30.171 | 121.786 | 59.644 | 1.00 | 106.89 | 8 |
| ATOM | 16411 | N | LEU | G | 965 | 28.997 | 120.044 | 58.828 | 1.00 | 84.86 | 7 |
| ATOM | 16412 | CA | LEU | G | 965 | 28.916 | 120.599 | 57.480 | 1.00 | 84.27 | 6 |
| ATOM | 16413 | CB | LEU | G | 965 | 28.087 | 119.682 | 56.579 | 1.00 | 49.14 | 6 |
| ATOM | 16414 | CG | LEU | G | 965 | 28.766 | 119.012 | 55.383 | 1.00 | 47.71 | 6 |
| ATOM | 16415 | CD1 | LEU | G | 965 | 28.853 | 119.988 | 54.234 | 1.00 | 47.32 | 6 |
| ATOM | 16416 | CD2 | LEU | G | 965 | 30.144 | 118.507 | 55.786 | 1.00 | 47.30 | 6 |
| ATOM | 16417 | C | LEU | G | 965 | 28.184 | 121.911 | 57.706 | 1.00 | 84.16 | 6 |
| ATOM | 16418 | O | LEU | G | 965 | 27.655 | 122.127 | 58.795 | 1.00 | 84.72 | 8 |
| ATOM | 16419 | N | GLU | G | 966 | 28.128 | 122.784 | 56.708 | 1.00 | 42.27 | 7 |
| ATOM | 16420 | CA | GLU | G | 966 | 27.445 | 124.070 | 56.904 | 1.00 | 42.38 | 6 |
| ATOM | 16421 | CB | GLU | G | 966 | 25.981 | 123.871 | 57.303 | 1.00 | 91.78 | 6 |
| ATOM | 16422 | CG | GLU | G | 966 | 25.298 | 125.180 | 57.596 | 1.00 | 94.62 | 6 |
| ATOM | 16423 | CD | GLU | G | 966 | 25.716 | 126.263 | 56.616 | 1.00 | 96.45 | 6 |
| ATOM | 16424 | OE1 | GLU | G | 966 | 25.489 | 126.086 | 55.401 | 1.00 | 97.78 | 8 |
| ATOM | 16425 | OE2 | GLU | G | 966 | 26.279 | 127.285 | 57.058 | 1.00 | 97.51 | 8 |
| ATOM | 16426 | C | GLU | G | 966 | 28.160 | 124.889 | 57.979 | 1.00 | 41.33 | 6 |
| ATOM | 16427 | O | GLU | G | 966 | 28.475 | 126.045 | 57.757 | 1.00 | 40.16 | 8 |
| ATOM | 16428 | N | GLU | G | 967 | 28.377 | 124.313 | 59.158 | 1.00 | 36.77 | 7 |
| ATOM | 16429 | CA | GLU | G | 967 | 29.135 | 125.021 | 60.166 | 1.00 | 35.92 | 6 |
| ATOM | 16430 | CB | GLU | G | 967 | 29.340 | 124.181 | 61.421 | 1.00 | 51.25 | 6 |
| ATOM | 16431 | CG | GLU | G | 967 | 28.196 | 124.274 | 62.402 | 1.00 | 51.43 | 6 |
| ATOM | 16432 | CD | GLU | G | 967 | 28.528 | 123.686 | 63.756 | 1.00 | 51.72 | 6 |
| ATOM | 16433 | OE1 | GLU | G | 967 | 29.613 | 123.083 | 63.915 | 1.00 | 51.54 | 8 |
| ATOM | 16434 | OE2 | GLU | G | 967 | 27.693 | 123.828 | 64.670 | 1.00 | 53.39 | 8 |
| ATOM | 16435 | C | GLU | G | 967 | 30.454 | 125.196 | 59.435 | 1.00 | 35.75 | 6 |
| ATOM | 16436 | O | GLU | G | 967 | 31.121 | 126.227 | 59.534 | 1.00 | 35.02 | 8 |
| ATOM | 16437 | N | ALA | G | 968 | 30.820 | 124.170 | 58.679 | 1.00 | 38.97 | 7 |
| ATOM | 16438 | CA | ALA | G | 968 | 32.030 | 124.236 | 57.885 | 1.00 | 40.14 | 6 |
| ATOM | 16439 | CB | ALA | G | 968 | 32.121 | 123.028 | 56.962 | 1.00 | 96.20 | 6 |
| ATOM | 16440 | C | ALA | G | 968 | 31.848 | 125.509 | 57.073 | 1.00 | 41.00 | 6 |
| ATOM | 16441 | O | ALA | G | 968 | 32.798 | 126.254 | 56.836 | 1.00 | 40.00 | 8 |
| ATOM | 16442 | N | ASP | G | 969 | 30.600 | 125.746 | 56.670 | 1.00 | 59.01 | 7 |
| ATOM | 16443 | CA | ASP | G | 969 | 30.232 | 126.922 | 55.888 | 1.00 | 61.20 | 6 |
| ATOM | 16444 | CB | ASP | G | 969 | 28.871 | 126.725 | 55.210 | 1.00 | 93.07 | 6 |
| ATOM | 16445 | CG | ASP | G | 969 | 28.984 | 125.999 | 53.892 | 1.00 | 95.50 | 6 |
| ATOM | 16446 | OD1 | ASP | G | 969 | 29.627 | 126.548 | 52.971 | 1.00 | 96.94 | 8 |
| ATOM | 16447 | OD2 | ASP | G | 969 | 28.437 | 124.882 | 53.776 | 1.00 | 97.48 | 8 |
| ATOM | 16448 | C | ASP | G | 969 | 30.204 | 128.185 | 56.737 | 1.00 | 61.41 | 6 |
| ATOM | 16449 | O | ASP | G | 969 | 31.253 | 128.742 | 56.997 | 1.00 | 60.85 | 8 |
| ATOM | 16450 | N | ARG | G | 970 | 29.016 | 128.612 | 57.175 | 1.00 | 70.79 | 7 |
| ATOM | 16451 | CA | ARG | G | 970 | 28.854 | 129.832 | 57.968 | 1.00 | 70.46 | 6 |
| ATOM | 16452 | CB | ARG | G | 970 | 28.246 | 129.543 | 59.355 | 1.00 | 74.41 | 6 |
| ATOM | 16453 | CG | ARG | G | 970 | 29.233 | 129.035 | 60.404 | 1.00 | 76.46 | 6 |
| ATOM | 16454 | CD | ARG | G | 970 | 28.655 | 128.957 | 61.822 | 1.00 | 76.91 | 6 |
| ATOM | 16455 | NE | ARG | G | 970 | 29.668 | 128.497 | 62.779 | 1.00 | 77.99 | 7 |
| ATOM | 16456 | CZ | ARG | G | 970 | 29.459 | 128.318 | 64.081 | 1.00 | 79.41 | 6 |
| ATOM | 16457 | NH1 | ARG | G | 970 | 28.268 | 128.559 | 64.617 | 1.00 | 79.74 | 7 |
| ATOM | 16458 | NH2 | ARG | G | 970 | 30.448 | 127.892 | 64.852 | 1.00 | 79.74 | 7 |
| ATOM | 16459 | C | ARG | G | 970 | 30.197 | 130.524 | 58.117 | 1.00 | 70.09 | 6 |
| ATOM | 16460 | O | ARG | G | 970 | 30.385 | 131.605 | 57.574 | 1.00 | 71.11 | 8 |
| ATOM | 16461 | N | LYS | G | 971 | 31.133 | 129.881 | 58.822 | 1.00 | 36.18 | 7 |
| ATOM | 16462 | CA | LYS | G | 971 | 32.486 | 130.413 | 59.046 | 1.00 | 36.61 | 6 |
| ATOM | 16463 | CB | LYS | G | 971 | 33.105 | 129.788 | 60.301 | 1.00 | 73.91 | 6 |
| ATOM | 16464 | CG | LYS | G | 971 | 32.341 | 130.102 | 61.587 | 1.00 | 75.60 | 6 |
| ATOM | 16465 | CD | LYS | G | 971 | 33.044 | 129.546 | 62.813 | 1.00 | 75.54 | 6 |
| ATOM | 16466 | CE | LYS | G | 971 | 32.385 | 130.050 | 64.079 | 1.00 | 76.02 | 6 |
| ATOM | 16467 | NZ | LYS | G | 971 | 33.199 | 129.734 | 65.278 | 1.00 | 75.83 | 7 |
| ATOM | 16468 | C | LYS | G | 971 | 33.402 | 130.175 | 57.846 | 1.00 | 36.88 | 6 |
| ATOM | 16469 | O | LYS | G | 971 | 34.606 | 129.955 | 57.994 | 1.00 | 35.74 | 8 |
| ATOM | 16470 | N | LEU | G | 972 | 32.789 | 130.232 | 56.663 | 1.00 | 61.28 | 7 |
| ATOM | 16471 | CA | LEU | G | 972 | 33.442 | 130.060 | 55.367 | 1.00 | 61.61 | 6 |
| ATOM | 16472 | CB | LEU | G | 972 | 32.740 | 128.959 | 54.562 | 1.00 | 79.55 | 6 |
| ATOM | 16473 | CG | LEU | G | 972 | 33.582 | 127.909 | 53.825 | 1.00 | 79.97 | 6 |
| ATOM | 16474 | CD1 | LEU | G | 972 | 32.643 | 126.972 | 53.087 | 1.00 | 79.85 | 6 |
| ATOM | 16475 | CD2 | LEU | G | 972 | 34.552 | 128.566 | 52.853 | 1.00 | 79.58 | 6 |
| ATOM | 16476 | C | LEU | G | 972 | 33.258 | 131.411 | 54.667 | 1.00 | 61.34 | 6 |
| ATOM | 16477 | O | LEU | G | 972 | 34.223 | 132.037 | 54.227 | 1.00 | 60.98 | 8 |
| ATOM | 16478 | N | ARG | G | 973 | 32.007 | 131.852 | 54.561 | 1.00 | 56.08 | 7 |
| ATOM | 16479 | CA | ARG | G | 973 | 31.716 | 133.152 | 53.967 | 1.00 | 56.75 | 6 |
| ATOM | 16480 | CB | ARG | G | 973 | 30.212 | 133.408 | 53.922 | 1.00 | 102.76 | 6 |
| ATOM | 16481 | CG | ARG | G | 973 | 29.465 | 132.463 | 53.019 | 1.00 | 103.34 | 6 |
| ATOM | 16482 | CD | ARG | G | 973 | 27.964 | 132.679 | 53.099 | 1.00 | 104.23 | 6 |
| ATOM | 16483 | NE | ARG | G | 973 | 27.265 | 131.758 | 52.207 | 1.00 | 106.13 | 7 |
| ATOM | 16484 | CZ | ARG | G | 973 | 25.945 | 131.673 | 52.096 | 1.00 | 106.52 | 6 |
| ATOM | 16485 | NH1 | ARG | G | 973 | 25.165 | 132.461 | 52.826 | 1.00 | 107.03 | 7 |
| ATOM | 16486 | NH2 | ARG | G | 973 | 25.407 | 130.794 | 51.258 | 1.00 | 107.13 | 7 |
| ATOM | 16487 | C | ARG | G | 973 | 32.372 | 134.132 | 54.917 | 1.00 | 56.49 | 6 |
| ATOM | 16488 | O | ARG | G | 973 | 32.635 | 135.269 | 54.582 | 1.00 | 56.40 | 8 |
| ATOM | 16489 | N | GLN | G | 974 | 32.616 | 133.671 | 56.130 | 1.00 | 49.87 | 7 |
| ATOM | 16490 | CA | GLN | G | 974 | 33.283 | 134.491 | 57.108 | 1.00 | 50.68 | 6 |
| ATOM | 16491 | CB | GLN | G | 974 | 33.125 | 133.904 | 58.515 | 1.00 | 69.44 | 6 |
| ATOM | 16492 | CG | GLN | G | 974 | 31.747 | 134.139 | 59.138 | 1.00 | 69.56 | 6 |
| ATOM | 16493 | CD | GLN | G | 974 | 31.676 | 133.683 | 60.586 | 1.00 | 69.08 | 6 |
| ATOM | 16494 | OE1 | GLN | G | 974 | 32.574 | 133.960 | 61.380 | 1.00 | 68.22 | 8 |
| ATOM | 16495 | NE2 | GLN | G | 974 | 30.596 | 132.994 | 60.937 | 1.00 | 69.06 | 7 |
| ATOM | 16496 | C | GLN | G | 974 | 34.746 | 134.489 | 56.704 | 1.00 | 51.23 | 6 |
| ATOM | 16497 | O | GLN | G | 974 | 35.567 | 135.157 | 57.316 | 1.00 | 51.28 | 8 |
| ATOM | 16498 | N | ILE | G | 975 | 35.070 | 133.725 | 55.668 | 1.00 | 73.01 | 7 |
| ATOM | 16499 | CA | ILE | G | 975 | 36.442 | 133.648 | 55.195 | 1.00 | 74.05 | 6 |
| ATOM | 16500 | CB | ILE | G | 975 | 36.955 | 132.174 | 55.138 | 1.00 | 66.18 | 6 |
| ATOM | 16501 | CG2 | ILE | G | 975 | 36.995 | 131.660 | 53.695 | 1.00 | 66.25 | 6 |
| ATOM | 16502 | CG1 | ILE | G | 975 | 38.344 | 132.084 | 55.778 | 1.00 | 66.26 | 6 |
| ATOM | 16503 | CD1 | ILE | G | 975 | 39.334 | 133.106 | 55.269 | 1.00 | 67.69 | 6 |
| ATOM | 16504 | C | ILE | G | 975 | 36.515 | 134.263 | 53.816 | 1.00 | 74.60 | 6 |
| ATOM | 16505 | O | ILE | G | 975 | 37.445 | 135.009 | 53.520 | 1.00 | 75.05 | 8 |
| ATOM | 16506 | N | GLU | G | 976 | 35.526 | 133.953 | 52.978 | 1.00 | 60.49 | 7 |
| ATOM | 16507 | CA | GLU | G | 976 | 35.482 | 134.483 | 51.617 | 1.00 | 61.71 | 6 |
| ATOM | 16508 | CB | GLU | G | 976 | 34.583 | 133.621 | 50.718 | 1.00 | 114.33 | 6 |
| ATOM | 16509 | CG | GLU | G | 976 | 34.937 | 132.127 | 50.690 | 1.00 | 115.06 | 6 |
| ATOM | 16510 | CD | GLU | G | 976 | 34.323 | 131.381 | 49.501 | 1.00 | 114.53 | 6 |
| ATOM | 16511 | OE1 | GLU | G | 976 | 33.257 | 131.803 | 49.002 | 1.00 | 113.15 | 8 |
| ATOM | 16512 | OE2 | GLU | G | 976 | 34.905 | 130.361 | 49.073 | 1.00 | 114.48 | 8 |
| ATOM | 16513 | C | GLU | G | 976 | 34.979 | 135.926 | 51.627 | 1.00 | 61.73 | 6 |
| ATOM | 16514 | O | GLU | G | 976 | 34.806 | 136.539 | 50.575 | 1.00 | 61.75 | 8 |
| ATOM | 16515 | N | GLN | G | 977 | 34.735 | 136.449 | 52.828 | 1.00 | 89.50 | 7 |
| ATOM | 16516 | CA | GLN | G | 977 | 34.290 | 137.829 | 53.032 | 1.00 | 89.42 | 6 |
| ATOM | 16517 | CB | GLN | G | 977 | 32.946 | 137.878 | 53.767 | 1.00 | 129.61 | 6 |
| ATOM | 16518 | CG | GLN | G | 977 | 31.729 | 137.620 | 52.876 | 1.00 | 131.76 | 6 |
| ATOM | 16519 | CD | GLN | G | 977 | 31.061 | 138.901 | 52.404 | 1.00 | 132.90 | 6 |
| ATOM | 16520 | OE1 | GLN | G | 977 | 31.715 | 139.799 | 51.877 | 1.00 | 134.36 | 8 |
| ATOM | 16521 | NE2 | GLN | G | 977 | 29.749 | 138.988 | 52.591 | 1.00 | 133.24 | 7 |
| ATOM | 16522 | C | GLN | G | 977 | 35.369 | 138.483 | 53.877 | 1.00 | 88.52 | 6 |
| ATOM | 16523 | O | GLN | G | 977 | 35.669 | 139.661 | 53.716 | 1.00 | 89.61 | 8 |
| ATOM | 16524 | N | ALA | G | 978 | 35.950 | 137.693 | 54.777 | 1.00 | 53.12 | 7 |
| ATOM | 16525 | CA | ALA | G | 978 | 37.041 | 138.147 | 55.636 | 1.00 | 50.88 | 6 |
| ATOM | 16526 | CB | ALA | G | 978 | 37.266 | 137.167 | 56.776 | 1.00 | 47.24 | 6 |
| ATOM | 16527 | C | ALA | G | 978 | 38.292 | 138.220 | 54.765 | 1.00 | 49.52 | 6 |
| ATOM | 16528 | O | ALA | G | 978 | 39.414 | 137.974 | 55.220 | 1.00 | 48.83 | 8 |
| ATOM | 16529 | N | TYR | G | 979 | 38.063 | 138.537 | 53.497 | 1.00 | 43.07 | 7 |
| ATOM | 16530 | CA | TYR | G | 979 | 39.110 | 138.674 | 52.503 | 1.00 | 42.64 | 6 |
| ATOM | 16531 | CB | TYR | G | 979 | 39.216 | 137.416 | 51.629 | 1.00 | 64.45 | 6 |
| ATOM | 16532 | CG | TYR | G | 979 | 40.275 | 137.502 | 50.543 | 1.00 | 63.51 | 6 |
| ATOM | 16533 | CD1 | TYR | G | 979 | 41.590 | 137.845 | 50.854 | 1.00 | 62.98 | 6 |
| ATOM | 16534 | CE1 | TYR | G | 979 | 42.562 | 137.945 | 49.862 | 1.00 | 64.04 | 6 |
| ATOM | 16535 | CD2 | TYR | G | 979 | 39.955 | 137.255 | 49.203 | 1.00 | 63.09 | 6 |
| ATOM | 16536 | CE2 | TYR | G | 979 | 40.922 | 137.353 | 48.196 | 1.00 | 63.46 | 6 |
| ATOM | 16537 | CZ | TYR | G | 979 | 42.227 | 137.703 | 48.533 | 1.00 | 64.43 | 6 |
| ATOM | 16538 | OH | TYR | G | 979 | 43.195 | 137.845 | 47.552 | 1.00 | 65.47 | 8 |
| ATOM | 16539 | C | TYR | G | 979 | 38.681 | 139.850 | 51.662 | 1.00 | 42.30 | 6 |
| ATOM | 16540 | O | TYR | G | 979 | 39.494 | 140.685 | 51.296 | 1.00 | 41.61 | 8 |
| ATOM | 16541 | N | GLU | G | 980 | 37.392 | 139.920 | 51.357 | 1.00 | 48.04 | 7 |
| ATOM | 16542 | CA | GLU | G | 980 | 36.884 | 141.034 | 50.572 | 1.00 | 48.99 | 6 |
| ATOM | 16543 | CB | GLU | G | 980 | 35.416 | 140.810 | 50.204 | 1.00 | 54.42 | 6 |
| ATOM | 16544 | CG | GLU | G | 980 | 35.198 | 139.531 | 49.407 | 1.00 | 54.26 | 6 |
| ATOM | 16545 | CD | GLU | G | 980 | 33.750 | 139.310 | 49.018 | 1.00 | 54.22 | 6 |
| ATOM | 16546 | OE1 | GLU | G | 980 | 32.893 | 139.196 | 49.922 | 1.00 | 52.78 | 8 |
| ATOM | 16547 | OE2 | GLU | G | 980 | 33.471 | 139.246 | 47.798 | 1.00 | 54.74 | 8 |
| ATOM | 16548 | C | GLU | G | 980 | 37.041 | 142.233 | 51.487 | 1.00 | 49.98 | 6 |
| ATOM | 16549 | O | GLU | G | 980 | 36.668 | 143.353 | 51.140 | 1.00 | 50.15 | 8 |
| ATOM | 16550 | N | MET | G | 981 | 37.610 | 141.957 | 52.662 | 1.00 | 58.41 | 7 |
| ATOM | 16551 | CA | MET | G | 981 | 37.885 | 142.941 | 53.703 | 1.00 | 59.37 | 6 |
| ATOM | 16552 | CB | MET | G | 981 | 37.512 | 142.382 | 55.068 | 1.00 | 62.35 | 6 |
| ATOM | 16553 | CG | MET | G | 981 | 36.037 | 142.107 | 55.230 | 1.00 | 61.41 | 6 |
| ATOM | 16554 | SD | MET | G | 981 | 35.134 | 143.533 | 55.807 | 1.00 | 61.27 | 16 |
| ATOM | 16555 | CE | MET | G | 981 | 35.630 | 143.514 | 57.569 | 1.00 | 60.29 | 6 |
| ATOM | 16556 | C | MET | G | 981 | 39.372 | 143.239 | 53.701 | 1.00 | 60.67 | 6 |
| ATOM | 16557 | O | MET | G | 981 | 40.118 | 142.720 | 52.871 | 1.00 | 60.62 | 8 |
| ATOM | 16558 | N | GLY | G | 982 | 39.810 | 144.061 | 54.646 | 1.00 | 136.85 | 7 |
| ATOM | 16559 | CA | GLY | G | 982 | 41.218 | 144.399 | 54.709 | 1.00 | 138.53 | 6 |
| ATOM | 16560 | C | GLY | G | 982 | 42.059 | 143.340 | 55.397 | 1.00 | 138.70 | 6 |
| ATOM | 16561 | O | GLY | G | 982 | 43.288 | 143.446 | 55.429 | 1.00 | 138.31 | 8 |
| ATOM | 16562 | N | PHE | G | 983 | 41.409 | 142.312 | 55.938 | 1.00 | 99.07 | 7 |
| ATOM | 16563 | CA | PHE | G | 983 | 42.136 | 141.262 | 56.643 | 1.00 | 99.25 | 6 |
| ATOM | 16564 | CB | PHE | G | 983 | 41.452 | 140.950 | 57.985 | 1.00 | 106.86 | 6 |
| ATOM | 16565 | CG | PHE | G | 983 | 41.470 | 142.111 | 58.938 | 1.00 | 107.86 | 6 |
| ATOM | 16566 | CD1 | PHE | G | 983 | 40.489 | 143.094 | 58.871 | 1.00 | 107.70 | 6 |
| ATOM | 16567 | CD2 | PHE | G | 983 | 42.538 | 142.292 | 59.815 | 1.00 | 108.55 | 6 |
| ATOM | 16568 | CE1 | PHE | G | 983 | 40.579 | 144.250 | 59.657 | 1.00 | 109.68 | 6 |
| ATOM | 16569 | CE2 | PHE | G | 983 | 42.638 | 143.442 | 60.604 | 1.00 | 109.22 | 6 |
| ATOM | 16570 | CZ | PHE | G | 983 | 41.658 | 144.424 | 60.523 | 1.00 | 110.07 | 6 |
| ATOM | 16571 | C | PHE | G | 983 | 42.380 | 139.986 | 55.855 | 1.00 | 98.41 | 6 |
| ATOM | 16572 | O | PHE | G | 983 | 41.688 | 139.679 | 54.881 | 1.00 | 97.87 | 8 |
| ATOM | 16573 | N | LEU | G | 984 | 43.396 | 139.257 | 56.300 | 1.00 | 74.49 | 7 |
| ATOM | 16574 | CA | LEU | G | 984 | 43.804 | 138.017 | 55.672 | 1.00 | 73.90 | 6 |
| ATOM | 16575 | CB | LEU | G | 984 | 42.699 | 136.975 | 55.747 | 1.00 | 35.43 | 6 |
| ATOM | 16576 | CG | LEU | G | 984 | 42.688 | 136.148 | 57.024 | 1.00 | 35.39 | 6 |
| ATOM | 16577 | CD1 | LEU | G | 984 | 42.017 | 134.808 | 56.735 | 1.00 | 35.23 | 6 |
| ATOM | 16578 | CD2 | LEU | G | 984 | 44.120 | 135.934 | 57.503 | 1.00 | 35.06 | 6 |
| ATOM | 16579 | C | LEU | G | 984 | 44.224 | 138.187 | 54.227 | 1.00 | 74.50 | 6 |
| ATOM | 16580 | O | LEU | G | 984 | 43.541 | 138.837 | 53.425 | 1.00 | 74.35 | 8 |
| ATOM | 16581 | N | THR | G | 985 | 45.358 | 137.571 | 53.918 | 1.00 | 103.35 | 7 |
| ATOM | 16582 | CA | THR | G | 985 | 45.956 | 137.592 | 52.595 | 1.00 | 102.52 | 6 |
| ATOM | 16583 | CB | THR | G | 985 | 47.474 | 137.619 | 52.718 | 1.00 | 63.64 | 6 |
| ATOM | 16584 | OG1 | THR | G | 985 | 48.058 | 137.154 | 51.497 | 1.00 | 63.76 | 8 |
| ATOM | 16585 | CG2 | THR | G | 985 | 47.926 | 136.727 | 53.876 | 1.00 | 62.10 | 6 |
| ATOM | 16586 | C | THR | G | 985 | 45.562 | 136.333 | 51.842 | 1.00 | 103.15 | 6 |
| ATOM | 16587 | O | THR | G | 985 | 45.472 | 135.267 | 52.441 | 1.00 | 104.38 | 8 |
| ATOM | 16588 | N | ASP | G | 986 | 45.336 | 136.446 | 50.535 | 1.00 | 40.12 | 7 |
| ATOM | 16589 | CA | ASP | G | 986 | 44.954 | 135.283 | 49.725 | 1.00 | 40.26 | 6 |
| ATOM | 16590 | CB | ASP | G | 986 | 45.248 | 135.544 | 48.237 | 1.00 | 48.74 | 6 |
| ATOM | 16591 | CG | ASP | G | 986 | 44.947 | 134.333 | 47.349 | 1.00 | 48.21 | 6 |
| ATOM | 16592 | OD1 | ASP | G | 986 | 45.667 | 133.316 | 47.456 | 1.00 | 48.01 | 8 |
| ATOM | 16593 | OD2 | ASP | G | 986 | 43.992 | 134.397 | 46.542 | 1.00 | 46.65 | 8 |
| ATOM | 16594 | C | ASP | G | 986 | 45.682 | 134.017 | 50.191 | 1.00 | 40.96 | 6 |
| ATOM | 16595 | O | ASP | G | 986 | 45.047 | 133.019 | 50.526 | 1.00 | 40.37 | 8 |
| ATOM | 16596 | N | ARG | G | 987 | 47.012 | 134.076 | 50.212 | 1.00 | 72.46 | 7 |
| ATOM | 16597 | CA | ARG | G | 987 | 47.844 | 132.958 | 50.652 | 1.00 | 73.44 | 6 |
| ATOM | 16598 | CB | ARG | G | 987 | 49.291 | 133.435 | 50.835 | 1.00 | 162.20 | 6 |
| ATOM | 16599 | CG | ARG | G | 987 | 50.253 | 132.405 | 51.421 | 1.00 | 164.64 | 6 |
| ATOM | 16600 | CD | ARG | G | 987 | 51.667 | 132.980 | 51.530 | 1.00 | 167.31 | 6 |
| ATOM | 16601 | NE | ARG | G | 987 | 52.593 | 132.073 | 52.206 | 1.00 | 169.35 | 7 |
| ATOM | 16602 | CZ | ARG | G | 987 | 53.890 | 132.315 | 52.379 | 1.00 | 169.89 | 6 |
| ATOM | 16603 | NH1 | ARG | G | 987 | 54.429 | 133.441 | 51.926 | 1.00 | 169.74 | 7 |
| ATOM | 16604 | NH2 | ARG | G | 987 | 54.652 | 131.432 | 53.011 | 1.00 | 170.59 | 7 |
| ATOM | 16605 | C | ARG | G | 987 | 47.308 | 132.389 | 51.966 | 1.00 | 73.13 | 6 |
| ATOM | 16606 | O | ARG | G | 987 | 46.797 | 131.272 | 52.007 | 1.00 | 73.67 | 8 |
| ATOM | 16607 | N | GLU | G | 988 | 47.420 | 133.165 | 53.039 | 1.00 | 64.26 | 7 |
| ATOM | 16608 | CA | GLU | G | 988 | 46.942 | 132.730 | 54.346 | 1.00 | 64.14 | 6 |
| ATOM | 16609 | CB | GLU | G | 988 | 47.285 | 133.772 | 55.412 | 1.00 | 196.85 | 6 |
| ATOM | 16610 | CG | GLU | G | 988 | 48.776 | 134.040 | 55.546 | 1.00 | 201.74 | 6 |
| ATOM | 16611 | CD | GLU | G | 988 | 49.588 | 132.763 | 55.682 | 1.00 | 203.98 | 6 |
| ATOM | 16612 | OE1 | GLU | G | 988 | 49.299 | 131.963 | 56.599 | 1.00 | 205.25 | 8 |
| ATOM | 16613 | OE2 | GLU | G | 988 | 50.521 | 132.560 | 54.874 | 1.00 | 204.76 | 8 |
| ATOM | 16614 | C | GLU | G | 988 | 45.442 | 132.497 | 54.318 | 1.00 | 62.52 | 6 |
| ATOM | 16615 | O | GLU | G | 988 | 44.895 | 131.794 | 55.160 | 1.00 | 62.41 | 8 |
| ATOM | 16616 | N | ARG | G | 989 | 44.776 | 133.101 | 53.346 | 1.00 | 50.18 | 7 |
| ATOM | 16617 | CA | ARG | G | 989 | 43.343 | 132.938 | 53.216 | 1.00 | 48.71 | 6 |
| ATOM | 16618 | CB | ARG | G | 989 | 42.800 | 133.827 | 52.107 | 1.00 | 78.53 | 6 |
| ATOM | 16619 | CG | ARG | G | 989 | 41.412 | 133.452 | 51.682 | 1.00 | 79.47 | 6 |
| ATOM | 16620 | CD | ARG | G | 989 | 40.959 | 134.300 | 50.526 | 1.00 | 80.65 | 6 |
| ATOM | 16621 | NE | ARG | G | 989 | 39.707 | 133.797 | 49.983 | 1.00 | 81.51 | 7 |
| ATOM | 16622 | CZ | ARG | G | 989 | 39.588 | 132.627 | 49.372 | 1.00 | 82.43 | 6 |
| ATOM | 16623 | NH1 | ARG | G | 989 | 40.650 | 131.839 | 49.221 | 1.00 | 82.82 | 7 |
| ATOM | 16624 | NH2 | ARG | G | 989 | 38.403 | 132.244 | 48.920 | 1.00 | 83.11 | 7 |
| ATOM | 16625 | C | ARG | G | 989 | 43.125 | 131.487 | 52.863 | 1.00 | 47.79 | 6 |
| ATOM | 16626 | O | ARG | G | 989 | 42.602 | 130.728 | 53.661 | 1.00 | 46.81 | 8 |
| ATOM | 16627 | N | TYR | G | 990 | 43.524 | 131.106 | 51.657 | 1.00 | 100.39 | 7 |
| ATOM | 16628 | CA | TYR | G | 990 | 43.388 | 129.723 | 51.230 | 1.00 | 99.27 | 6 |
| ATOM | 16629 | CB | TYR | G | 990 | 44.283 | 129.459 | 50.021 | 1.00 | 48.00 | 6 |
| ATOM | 16630 | CG | TYR | G | 990 | 44.767 | 128.030 | 49.903 | 1.00 | 47.49 | 6 |
| ATOM | 16631 | CD1 | TYR | G | 990 | 44.056 | 127.088 | 49.159 | 1.00 | 46.75 | 6 |
| ATOM | 16632 | CE1 | TYR | G | 990 | 44.493 | 125.777 | 49.058 | 1.00 | 46.56 | 6 |
| ATOM | 16633 | CD2 | TYR | G | 990 | 45.934 | 127.620 | 50.548 | 1.00 | 47.38 | 6 |
| ATOM | 16634 | CE2 | TYR | G | 990 | 46.379 | 126.315 | 50.458 | 1.00 | 47.53 | 6 |
| ATOM | 16635 | CZ | TYR | G | 990 | 45.656 | 125.397 | 49.711 | 1.00 | 46.98 | 6 |
| ATOM | 16636 | OH | TYR | G | 990 | 46.099 | 124.098 | 49.619 | 1.00 | 48.16 | 8 |
| ATOM | 16637 | C | TYR | G | 990 | 43.809 | 128.823 | 52.386 | 1.00 | 99.19 | 6 |
| ATOM | 16638 | O | TYR | G | 990 | 43.080 | 127.907 | 52.763 | 1.00 | 100.55 | 8 |
| ATOM | 16639 | N | ASP | G | 991 | 44.985 | 129.097 | 52.948 | 1.00 | 45.47 | 7 |
| ATOM | 16640 | CA | ASP | G | 991 | 45.508 | 128.315 | 54.061 | 1.00 | 44.34 | 6 |
| ATOM | 16641 | CB | ASP | G | 991 | 46.802 | 128.933 | 54.558 | 1.00 | 71.37 | 6 |
| ATOM | 16642 | CG | ASP | G | 991 | 47.986 | 128.454 | 53.783 | 1.00 | 71.72 | 6 |
| ATOM | 16643 | OD1 | ASP | G | 991 | 47.813 | 128.194 | 52.572 | 1.00 | 71.11 | 8 |
| ATOM | 16644 | OD2 | ASP | G | 991 | 49.081 | 128.339 | 54.378 | 1.00 | 70.92 | 8 |
| ATOM | 16645 | C | ASP | G | 991 | 44.535 | 128.154 | 55.224 | 1.00 | 43.77 | 6 |
| ATOM | 16646 | O | ASP | G | 991 | 44.849 | 127.505 | 56.224 | 1.00 | 44.24 | 8 |
| ATOM | 16647 | N | GLN | G | 992 | 43.360 | 128.761 | 55.090 | 1.00 | 67.50 | 7 |
| ATOM | 16648 | CA | GLN | G | 992 | 42.311 | 128.673 | 56.097 | 1.00 | 66.59 | 6 |
| ATOM | 16649 | CB | GLN | G | 992 | 41.524 | 129.984 | 56.175 | 1.00 | 87.39 | 6 |
| ATOM | 16650 | CG | GLN | G | 992 | 40.962 | 130.293 | 57.541 | 1.00 | 88.50 | 6 |
| ATOM | 16651 | CD | GLN | G | 992 | 42.054 | 130.476 | 58.579 | 1.00 | 89.39 | 6 |
| ATOM | 16652 | OE1 | GLN | G | 992 | 42.940 | 131.318 | 58.427 | 1.00 | 89.27 | 8 |
| ATOM | 16653 | NE2 | GLN | G | 992 | 41.994 | 129.685 | 59.641 | 1.00 | 90.51 | 7 |
| ATOM | 16654 | C | GLN | G | 992 | 41.417 | 127.576 | 55.561 | 1.00 | 64.89 | 6 |
| ATOM | 16655 | O | GLN | G | 992 | 41.330 | 126.498 | 56.142 | 1.00 | 64.31 | 8 |
| ATOM | 16656 | N | VAL | G | 993 | 40.788 | 127.867 | 54.422 | 1.00 | 41.54 | 7 |
| ATOM | 16657 | CA | VAL | G | 993 | 39.888 | 126.945 | 53.728 | 1.00 | 39.16 | 6 |
| ATOM | 16658 | CB | VAL | G | 993 | 39.761 | 127.310 | 52.241 | 1.00 | 93.99 | 6 |
| ATOM | 16659 | CG1 | VAL | G | 993 | 38.581 | 126.573 | 51.626 | 1.00 | 96.58 | 6 |
| ATOM | 16660 | CG2 | VAL | G | 993 | 39.609 | 128.816 | 52.088 | 1.00 | 94.44 | 6 |
| ATOM | 16661 | C | VAL | G | 993 | 40.393 | 125.512 | 53.816 | 1.00 | 36.63 | 6 |
| ATOM | 16662 | O | VAL | G | 993 | 39.606 | 124.561 | 53.844 | 1.00 | 35.42 | 8 |
| ATOM | 16663 | N | ILE | G | 994 | 41.713 | 125.367 | 53.849 | 1.00 | 39.68 | 7 |
| ATOM | 16664 | CA | ILE | G | 994 | 42.310 | 124.059 | 53.955 | 1.00 | 37.87 | 6 |
| ATOM | 16665 | CB | ILE | G | 994 | 43.762 | 124.050 | 53.473 | 1.00 | 46.80 | 6 |
| ATOM | 16666 | CG2 | ILE | G | 994 | 44.459 | 122.772 | 53.932 | 1.00 | 46.67 | 6 |
| ATOM | 16667 | CG1 | ILE | G | 994 | 43.796 | 124.180 | 51.948 | 1.00 | 45.87 | 6 |
| ATOM | 16668 | CD1 | ILE | G | 994 | 43.123 | 123.050 | 51.185 | 1.00 | 46.35 | 6 |
| ATOM | 16669 | C | ILE | G | 994 | 42.266 | 123.649 | 55.405 | 1.00 | 37.18 | 6 |
| ATOM | 16670 | O | ILE | G | 994 | 41.694 | 122.619 | 55.731 | 1.00 | 37.74 | 8 |
| ATOM | 16671 | N | GLN | G | 995 | 42.852 | 124.451 | 56.284 | 1.00 | 54.87 | 7 |
| ATOM | 16672 | CA | GLN | G | 995 | 42.839 | 124.096 | 57.698 | 1.00 | 55.60 | 6 |
| ATOM | 16673 | CB | GLN | G | 995 | 43.404 | 125.227 | 58.558 | 1.00 | 72.70 | 6 |
| ATOM | 16674 | CG | GLN | G | 995 | 43.653 | 124.826 | 60.008 | 1.00 | 74.66 | 6 |
| ATOM | 16675 | CD | GLN | G | 995 | 44.126 | 125.986 | 60.854 | 1.00 | 75.97 | 6 |
| ATOM | 16676 | OE1 | GLN | G | 995 | 45.143 | 126.616 | 60.554 | 1.00 | 76.13 | 8 |
| ATOM | 16677 | NE2 | GLN | G | 995 | 43.387 | 126.282 | 61.917 | 1.00 | 76.58 | 7 |
| ATOM | 16678 | C | GLN | G | 995 | 41.402 | 123.809 | 58.109 | 1.00 | 54.89 | 6 |
| ATOM | 16679 | O | GLN | G | 995 | 41.131 | 122.890 | 58.883 | 1.00 | 54.68 | 8 |
| ATOM | 16680 | N | LEU | G | 996 | 40.481 | 124.604 | 57.578 | 1.00 | 44.32 | 7 |
| ATOM | 16681 | CA | LEU | G | 996 | 39.064 | 124.428 | 57.865 | 1.00 | 41.61 | 6 |
| ATOM | 16682 | CB | LEU | G | 996 | 38.231 | 125.439 | 57.073 | 1.00 | 13.87 | 6 |
| ATOM | 16683 | CG | LEU | G | 996 | 36.718 | 125.255 | 57.122 | 1.00 | 13.87 | 6 |
| ATOM | 16684 | CD1 | LEU | G | 996 | 36.220 | 125.289 | 58.562 | 1.00 | 13.87 | 6 |
| ATOM | 16685 | CD2 | LEU | G | 996 | 36.077 | 126.339 | 56.290 | 1.00 | 13.87 | 6 |
| ATOM | 16686 | C | LEU | G | 996 | 38.646 | 123.016 | 57.480 | 1.00 | 40.91 | 6 |
| ATOM | 16687 | O | LEU | G | 996 | 38.664 | 122.109 | 58.309 | 1.00 | 40.33 | 8 |
| ATOM | 16688 | N | TRP | G | 997 | 38.292 | 122.835 | 56.210 | 1.00 | 41.58 | 7 |
| ATOM | 16689 | CA | TRP | G | 997 | 37.853 | 121.541 | 55.737 | 1.00 | 40.97 | 6 |
| ATOM | 16690 | CB | TRP | G | 997 | 37.819 | 121.512 | 54.211 | 1.00 | 27.79 | 6 |
| ATOM | 16691 | CG | TRP | G | 997 | 36.620 | 122.245 | 53.646 | 1.00 | 24.48 | 6 |
| ATOM | 16692 | CD2 | TRP | G | 997 | 35.239 | 121.896 | 53.826 | 1.00 | 23.12 | 6 |
| ATOM | 16693 | CE2 | TRP | G | 997 | 34.470 | 122.860 | 53.135 | 1.00 | 22.07 | 6 |
| ATOM | 16694 | CE3 | TRP | G | 997 | 34.576 | 120.859 | 54.504 | 1.00 | 23.69 | 6 |
| ATOM | 16695 | CD1 | TRP | G | 997 | 36.626 | 123.377 | 52.874 | 1.00 | 22.58 | 6 |
| ATOM | 16696 | NE1 | TRP | G | 997 | 35.340 | 123.751 | 52.564 | 1.00 | 21.39 | 7 |
| ATOM | 16697 | CZ2 | TRP | G | 997 | 33.069 | 122.819 | 53.104 | 1.00 | 22.49 | 6 |
| ATOM | 16698 | CZ3 | TRP | G | 997 | 33.176 | 120.817 | 54.474 | 1.00 | 23.06 | 6 |
| ATOM | 16699 | CH2 | TRP | G | 997 | 32.443 | 121.792 | 53.777 | 1.00 | 23.23 | 6 |
| ATOM | 16700 | C | TRP | G | 997 | 38.703 | 120.414 | 56.283 | 1.00 | 43.11 | 6 |
| ATOM | 16701 | O | TRP | G | 997 | 38.178 | 119.360 | 56.630 | 1.00 | 44.67 | 8 |
| ATOM | 16702 | N | THR | G | 998 | 40.010 | 120.627 | 56.396 | 1.00 | 20.39 | 7 |
| ATOM | 16703 | CA | THR | G | 998 | 40.857 | 119.559 | 56.916 | 1.00 | 22.56 | 6 |
| ATOM | 16704 | CB | THR | G | 998 | 42.329 | 119.960 | 57.050 | 1.00 | 67.19 | 6 |
| ATOM | 16705 | OG1 | THR | G | 998 | 42.854 | 120.323 | 55.767 | 1.00 | 69.15 | 8 |
| ATOM | 16706 | CG2 | THR | G | 998 | 43.133 | 118.787 | 57.587 | 1.00 | 67.83 | 6 |
| ATOM | 16707 | C | THR | G | 998 | 40.351 | 119.192 | 58.281 | 1.00 | 23.80 | 6 |
| ATOM | 16708 | O | THR | G | 998 | 40.033 | 118.044 | 58.526 | 1.00 | 23.47 | 8 |
| ATOM | 16709 | N | GLU | G | 999 | 40.262 | 120.183 | 59.159 | 1.00 | 38.13 | 7 |
| ATOM | 16710 | CA | GLU | G | 999 | 39.789 | 119.976 | 60.525 | 1.00 | 40.51 | 6 |
| ATOM | 16711 | CB | GLU | G | 999 | 39.807 | 121.305 | 61.304 | 1.00 | 54.18 | 6 |
| ATOM | 16712 | CG | GLU | G | 999 | 40.972 | 121.456 | 62.296 | 1.00 | 55.64 | 6 |
| ATOM | 16713 | CD | GLU | G | 999 | 41.647 | 122.829 | 62.238 | 1.00 | 56.29 | 6 |
| ATOM | 16714 | OE1 | GLU | G | 999 | 40.940 | 123.858 | 62.316 | 1.00 | 55.80 | 8 |
| ATOM | 16715 | OE2 | GLU | G | 999 | 42.893 | 122.875 | 62.122 | 1.00 | 56.56 | 8 |
| ATOM | 16716 | C | GLU | G | 999 | 38.395 | 119.355 | 60.607 | 1.00 | 40.73 | 6 |
| ATOM | 16717 | O | GLU | G | 999 | 38.190 | 118.397 | 61.344 | 1.00 | 40.13 | 8 |
| ATOM | 16718 | N | THR | G | 1000 | 37.434 | 119.888 | 59.862 | 1.00 | 47.03 | 7 |
| ATOM | 16719 | CA | THR | G | 1000 | 36.092 | 119.338 | 59.933 | 1.00 | 48.94 | 6 |
| ATOM | 16720 | CB | THR | G | 1000 | 35.084 | 120.153 | 59.117 | 1.00 | 63.26 | 6 |
| ATOM | 16721 | OG1 | THR | G | 1000 | 33.795 | 119.545 | 59.228 | 1.00 | 62.73 | 8 |
| ATOM | 16722 | CG2 | THR | G | 1000 | 35.478 | 120.198 | 57.658 | 1.00 | 63.98 | 6 |
| ATOM | 16723 | C | THR | G | 1000 | 36.086 | 117.912 | 59.433 | 1.00 | 50.13 | 6 |
| ATOM | 16724 | O | THR | G | 1000 | 35.512 | 117.032 | 60.064 | 1.00 | 51.34 | 8 |
| ATOM | 16725 | N | THR | G | 1001 | 36.730 | 117.677 | 58.299 | 1.00 | 83.66 | 7 |
| ATOM | 16726 | CA | THR | G | 1001 | 36.792 | 116.334 | 57.745 | 1.00 | 84.47 | 6 |
| ATOM | 16727 | CB | THR | G | 1001 | 37.897 | 116.217 | 56.683 | 1.00 | 44.60 | 6 |
| ATOM | 16728 | OG1 | THR | G | 1001 | 37.408 | 116.691 | 55.426 | 1.00 | 42.63 | 8 |
| ATOM | 16729 | CG2 | THR | G | 1001 | 38.344 | 114.784 | 56.541 | 1.00 | 43.77 | 6 |
| ATOM | 16730 | C | THR | G | 1001 | 37.087 | 115.351 | 58.865 | 1.00 | 84.97 | 6 |
| ATOM | 16731 | O | THR | G | 1001 | 36.440 | 114.318 | 58.977 | 1.00 | 84.92 | 8 |
| ATOM | 16732 | N | GLU | G | 1002 | 38.058 | 115.694 | 59.702 | 1.00 | 47.50 | 7 |
| ATOM | 16733 | CA | GLU | G | 1002 | 38.449 | 114.839 | 60.804 | 1.00 | 48.08 | 6 |
| ATOM | 16734 | CB | GLU | G | 1002 | 39.798 | 115.294 | 61.377 | 1.00 | 163.45 | 6 |
| ATOM | 16735 | CG | GLU | G | 1002 | 40.942 | 115.292 | 60.367 | 1.00 | 168.56 | 6 |
| ATOM | 16736 | CD | GLU | G | 1002 | 42.287 | 115.640 | 60.990 | 1.00 | 171.31 | 6 |
| ATOM | 16737 | OE1 | GLU | G | 1002 | 42.788 | 114.848 | 61.815 | 1.00 | 173.00 | 8 |
| ATOM | 16738 | OE2 | GLU | G | 1002 | 42.844 | 116.707 | 60.657 | 1.00 | 172.32 | 8 |
| ATOM | 16739 | C | GLU | G | 1002 | 37.411 | 114.791 | 61.918 | 1.00 | 47.16 | 6 |
| ATOM | 16740 | O | GLU | G | 1002 | 37.352 | 113.810 | 62.659 | 1.00 | 47.55 | 8 |
| ATOM | 16741 | N | LYS | G | 1003 | 36.587 | 115.831 | 62.028 | 1.00 | 68.69 | 7 |
| ATOM | 16742 | CA | LYS | G | 1003 | 35.569 | 115.906 | 63.079 | 1.00 | 68.11 | 6 |
| ATOM | 16743 | CB | LYS | G | 1003 | 35.285 | 117.370 | 63.426 | 1.00 | 179.69 | 6 |
| ATOM | 16744 | CG | LYS | G | 1003 | 36.486 | 118.147 | 63.966 | 1.00 | 183.17 | 6 |
| ATOM | 16745 | CD | LYS | G | 1003 | 36.860 | 117.745 | 65.392 | 1.00 | 184.78 | 6 |
| ATOM | 16746 | CE | LYS | G | 1003 | 37.945 | 118.666 | 65.951 | 1.00 | 186.16 | 6 |
| ATOM | 16747 | NZ | LYS | G | 1003 | 38.268 | 118.382 | 67.378 | 1.00 | 185.20 | 7 |
| ATOM | 16748 | C | LYS | G | 1003 | 34.250 | 115.199 | 62.765 | 1.00 | 65.91 | 6 |
| ATOM | 16749 | O | LYS | G | 1003 | 33.421 | 115.024 | 63.649 | 1.00 | 67.12 | 8 |
| ATOM | 16750 | N | VAL | G | 1004 | 34.045 | 114.813 | 61.511 | 1.00 | 19.70 | 7 |
| ATOM | 16751 | CA | VAL | G | 1004 | 32.828 | 114.109 | 61.119 | 1.00 | 15.35 | 6 |
| ATOM | 16752 | CB | VAL | G | 1004 | 32.472 | 114.351 | 59.664 | 1.00 | 18.18 | 6 |
| ATOM | 16753 | CG1 | VAL | G | 1004 | 31.065 | 113.884 | 59.414 | 1.00 | 17.86 | 6 |
| ATOM | 16754 | CG2 | VAL | G | 1004 | 32.658 | 115.812 | 59.307 | 1.00 | 18.60 | 6 |
| ATOM | 16755 | C | VAL | G | 1004 | 33.135 | 112.631 | 61.263 | 1.00 | 13.87 | 6 |
| ATOM | 16756 | O | VAL | G | 1004 | 32.266 | 111.818 | 61.542 | 1.00 | 13.87 | 8 |
| ATOM | 16757 | N | THR | G | 1005 | 34.397 | 112.299 | 61.038 | 1.00 | 48.60 | 7 |
| ATOM | 16758 | CA | THR | G | 1005 | 34.888 | 110.946 | 61.165 | 1.00 | 48.72 | 6 |
| ATOM | 16759 | CB | THR | G | 1005 | 36.372 | 110.920 | 60.928 | 1.00 | 42.42 | 6 |
| ATOM | 16760 | OG1 | THR | G | 1005 | 36.598 | 110.923 | 59.522 | 1.00 | 43.69 | 8 |
| ATOM | 16761 | CG2 | THR | G | 1005 | 37.011 | 109.707 | 61.565 | 1.00 | 41.46 | 6 |
| ATOM | 16762 | C | THR | G | 1005 | 34.621 | 110.475 | 62.570 | 1.00 | 48.57 | 6 |
| ATOM | 16763 | O | THR | G | 1005 | 33.823 | 109.567 | 62.777 | 1.00 | 48.94 | 8 |
| ATOM | 16764 | N | GLN | G | 1006 | 35.302 | 111.099 | 63.532 | 1.00 | 45.99 | 7 |
| ATOM | 16765 | CA | GLN | G | 1006 | 35.140 | 110.770 | 64.945 | 1.00 | 44.76 | 6 |
| ATOM | 16766 | CB | GLN | G | 1006 | 35.882 | 111.788 | 65.811 | 1.00 | 113.28 | 6 |
| ATOM | 16767 | CG | GLN | G | 1006 | 35.703 | 111.568 | 67.302 | 1.00 | 117.54 | 6 |
| ATOM | 16768 | CD | GLN | G | 1006 | 36.327 | 112.673 | 68.126 | 1.00 | 118.86 | 6 |
| ATOM | 16769 | OE1 | GLN | G | 1006 | 35.954 | 113.840 | 68.000 | 1.00 | 118.93 | 8 |
| ATOM | 16770 | NE2 | GLN | G | 1006 | 37.281 | 112.312 | 68.979 | 1.00 | 119.91 | 7 |
| ATOM | 16771 | C | GLN | G | 1006 | 33.652 | 110.787 | 65.283 | 1.00 | 42.67 | 6 |
| ATOM | 16772 | O | GLN | G | 1006 | 33.211 | 110.183 | 66.259 | 1.00 | 41.89 | 8 |
| ATOM | 16773 | N | ALA | G | 1007 | 32.888 | 111.504 | 64.470 | 1.00 | 24.56 | 7 |
| ATOM | 16774 | CA | ALA | G | 1007 | 31.459 | 111.584 | 64.648 | 1.00 | 22.84 | 6 |
| ATOM | 16775 | CB | ALA | G | 1007 | 30.897 | 112.756 | 63.873 | 1.00 | 67.16 | 6 |
| ATOM | 16776 | C | ALA | G | 1007 | 30.935 | 110.273 | 64.092 | 1.00 | 21.77 | 6 |
| ATOM | 16777 | O | ALA | G | 1007 | 30.696 | 109.342 | 64.841 | 1.00 | 20.96 | 8 |
| ATOM | 16778 | N | VAL | G | 1008 | 30.779 | 110.186 | 62.776 | 1.00 | 37.05 | 7 |
| ATOM | 16779 | CA | VAL | G | 1008 | 30.298 | 108.958 | 62.152 | 1.00 | 37.60 | 6 |
| ATOM | 16780 | CB | VAL | G | 1008 | 30.568 | 108.953 | 60.650 | 1.00 | 79.15 | 6 |
| ATOM | 16781 | CG1 | VAL | G | 1008 | 32.016 | 109.333 | 60.381 | 1.00 | 80.90 | 6 |
| ATOM | 16782 | CG2 | VAL | G | 1008 | 30.262 | 107.570 | 60.080 | 1.00 | 80.31 | 6 |
| ATOM | 16783 | C | VAL | G | 1008 | 30.970 | 107.717 | 62.725 | 1.00 | 36.09 | 6 |
| ATOM | 16784 | O | VAL | G | 1008 | 30.312 | 106.773 | 63.133 | 1.00 | 34.76 | 8 |
| ATOM | 16785 | N | PHE | G | 1009 | 32.292 | 107.722 | 62.757 | 1.00 | 25.63 | 7 |
| ATOM | 16786 | CA | PHE | G | 1009 | 33.005 | 106.572 | 63.260 | 1.00 | 26.46 | 6 |
| ATOM | 16787 | CB | PHE | G | 1009 | 34.500 | 106.663 | 62.956 | 1.00 | 26.89 | 6 |
| ATOM | 16788 | CG | PHE | G | 1009 | 35.133 | 105.329 | 62.848 | 1.00 | 24.42 | 6 |
| ATOM | 16789 | CD1 | PHE | G | 1009 | 35.224 | 104.695 | 61.621 | 1.00 | 23.72 | 6 |
| ATOM | 16790 | CD2 | PHE | G | 1009 | 35.467 | 104.619 | 63.989 | 1.00 | 23.78 | 6 |
| ATOM | 16791 | CE1 | PHE | G | 1009 | 35.624 | 103.377 | 61.542 | 1.00 | 23.84 | 6 |
| ATOM | 16792 | CE2 | PHE | G | 1009 | 35.867 | 103.306 | 63.918 | 1.00 | 23.61 | 6 |
| ATOM | 16793 | CZ | PHE | G | 1009 | 35.941 | 102.681 | 62.692 | 1.00 | 24.04 | 6 |
| ATOM | 16794 | C | PHE | G | 1009 | 32.820 | 106.352 | 64.746 | 1.00 | 28.58 | 6 |
| ATOM | 16795 | O | PHE | G | 1009 | 33.691 | 105.800 | 65.411 | 1.00 | 29.23 | 8 |
| ATOM | 16796 | N | ASN | G | 1010 | 31.684 | 106.765 | 65.280 | 1.00 | 24.41 | 7 |
| ATOM | 16797 | CA | ASN | G | 1010 | 31.458 | 106.597 | 66.700 | 1.00 | 27.31 | 6 |
| ATOM | 16798 | CB | ASN | G | 1010 | 32.334 | 107.593 | 67.473 | 1.00 | 65.80 | 6 |
| ATOM | 16799 | CG | ASN | G | 1010 | 31.555 | 108.438 | 68.456 | 1.00 | 70.69 | 6 |
| ATOM | 16800 | OD1 | ASN | G | 1010 | 30.889 | 107.918 | 69.354 | 1.00 | 73.17 | 8 |
| ATOM | 16801 | ND2 | ASN | G | 1010 | 31.651 | 109.757 | 68.304 | 1.00 | 74.01 | 7 |
| ATOM | 16802 | C | ASN | G | 1010 | 29.979 | 106.794 | 66.958 | 1.00 | 27.09 | 6 |
| ATOM | 16803 | O | ASN | G | 1010 | 29.509 | 106.675 | 68.083 | 1.00 | 26.98 | 8 |
| ATOM | 16804 | N | ASN | G | 1011 | 29.238 | 107.106 | 65.900 | 1.00 | 26.98 | 7 |
| ATOM | 16805 | CA | ASN | G | 1011 | 27.803 | 107.247 | 66.042 | 1.00 | 27.01 | 6 |
| ATOM | 16806 | CB | ASN | G | 1011 | 27.136 | 107.491 | 64.680 | 1.00 | 34.41 | 6 |
| ATOM | 16807 | CG | ASN | G | 1011 | 25.653 | 107.074 | 64.648 | 1.00 | 33.50 | 6 |
| ATOM | 16808 | OD1 | ASN | G | 1011 | 24.993 | 106.962 | 65.684 | 1.00 | 31.38 | 8 |
| ATOM | 16809 | ND2 | ASN | G | 1011 | 25.128 | 106.864 | 63.442 | 1.00 | 33.53 | 7 |
| ATOM | 16810 | C | ASN | G | 1011 | 27.474 | 105.861 | 66.525 | 1.00 | 28.16 | 6 |
| ATOM | 16811 | O | ASN | G | 1011 | 26.616 | 105.678 | 67.393 | 1.00 | 28.30 | 8 |
| ATOM | 16812 | N | PHE | G | 1012 | 28.211 | 104.891 | 65.974 | 1.00 | 25.48 | 7 |
| ATOM | 16813 | CA | PHE | G | 1012 | 28.006 | 103.499 | 66.303 | 1.00 | 26.89 | 6 |
| ATOM | 16814 | CB | PHE | G | 1012 | 28.574 | 102.599 | 65.194 | 1.00 | 76.72 | 6 |
| ATOM | 16815 | CG | PHE | G | 1012 | 30.077 | 102.540 | 65.133 | 1.00 | 81.65 | 6 |
| ATOM | 16816 | CD1 | PHE | G | 1012 | 30.766 | 103.158 | 64.095 | 1.00 | 83.16 | 6 |
| ATOM | 16817 | CD2 | PHE | G | 1012 | 30.803 | 101.786 | 66.065 | 1.00 | 84.27 | 6 |
| ATOM | 16818 | CE1 | PHE | G | 1012 | 32.159 | 103.018 | 63.980 | 1.00 | 84.33 | 6 |
| ATOM | 16819 | CE2 | PHE | G | 1012 | 32.194 | 101.639 | 65.964 | 1.00 | 84.69 | 6 |
| ATOM | 16820 | CZ | PHE | G | 1012 | 32.870 | 102.254 | 64.917 | 1.00 | 84.73 | 6 |
| ATOM | 16821 | C | PHE | G | 1012 | 28.531 | 103.098 | 67.669 | 1.00 | 26.09 | 6 |
| ATOM | 16822 | O | PHE | G | 1012 | 27.961 | 102.237 | 68.334 | 1.00 | 26.39 | 8 |
| ATOM | 16823 | N | GLU | G | 1013 | 29.596 | 103.734 | 68.122 | 1.00 | 20.55 | 7 |
| ATOM | 16824 | CA | GLU | G | 1013 | 30.133 | 103.379 | 69.423 | 1.00 | 21.05 | 6 |
| ATOM | 16825 | CB | GLU | G | 1013 | 31.290 | 104.297 | 69.808 | 1.00 | 105.45 | 6 |
| ATOM | 16826 | CG | GLU | G | 1013 | 32.101 | 103.743 | 70.963 | 1.00 | 109.34 | 6 |
| ATOM | 16827 | CD | GLU | G | 1013 | 33.209 | 104.666 | 71.415 | 1.00 | 111.45 | 6 |
| ATOM | 16828 | OE1 | GLU | G | 1013 | 32.896 | 105.751 | 71.954 | 1.00 | 111.87 | 8 |
| ATOM | 16829 | OE2 | GLU | G | 1013 | 34.394 | 104.309 | 71.232 | 1.00 | 112.63 | 8 |
| ATOM | 16830 | C | GLU | G | 1013 | 29.067 | 103.429 | 70.507 | 1.00 | 19.55 | 6 |
| ATOM | 16831 | O | GLU | G | 1013 | 29.292 | 102.977 | 71.619 | 1.00 | 17.89 | 8 |
| ATOM | 16832 | N | GLU | G | 1014 | 27.898 | 103.967 | 70.198 | 1.00 | 34.82 | 7 |
| ATOM | 16833 | CA | GLU | G | 1014 | 26.849 | 104.039 | 71.206 | 1.00 | 36.24 | 6 |
| ATOM | 16834 | CB | GLU | G | 1014 | 26.915 | 105.378 | 71.939 | 1.00 | 110.80 | 6 |
| ATOM | 16835 | CG | GLU | G | 1014 | 28.309 | 105.733 | 72.414 | 1.00 | 114.48 | 6 |
| ATOM | 16836 | CD | GLU | G | 1014 | 28.320 | 106.961 | 73.279 | 1.00 | 116.34 | 6 |
| ATOM | 16837 | OE1 | GLU | G | 1014 | 27.643 | 107.945 | 72.914 | 1.00 | 117.29 | 8 |
| ATOM | 16838 | OE2 | GLU | G | 1014 | 29.012 | 106.946 | 74.319 | 1.00 | 117.78 | 8 |
| ATOM | 16839 | C | GLU | G | 1014 | 25.481 | 103.852 | 70.577 | 1.00 | 35.21 | 6 |
| ATOM | 16840 | O | GLU | G | 1014 | 24.452 | 104.195 | 71.147 | 1.00 | 34.67 | 8 |
| ATOM | 16841 | N | ASN | G | 1015 | 25.491 | 103.299 | 69.382 | 1.00 | 57.07 | 7 |
| ATOM | 16842 | CA | ASN | G | 1015 | 24.283 | 103.023 | 68.638 | 1.00 | 57.18 | 6 |
| ATOM | 16843 | CB | ASN | G | 1015 | 23.740 | 104.286 | 67.977 | 1.00 | 73.29 | 6 |
| ATOM | 16844 | CG | ASN | G | 1015 | 23.239 | 105.297 | 68.976 | 1.00 | 74.70 | 6 |
| ATOM | 16845 | OD1 | ASN | G | 1015 | 22.355 | 105.003 | 69.777 | 1.00 | 75.37 | 8 |
| ATOM | 16846 | ND2 | ASN | G | 1015 | 23.796 | 106.502 | 68.933 | 1.00 | 75.87 | 7 |
| ATOM | 16847 | C | ASN | G | 1015 | 24.825 | 102.113 | 67.582 | 1.00 | 56.29 | 6 |
| ATOM | 16848 | O | ASN | G | 1015 | 25.439 | 102.595 | 66.635 | 1.00 | 56.98 | 8 |
| ATOM | 16849 | N | TYR | G | 1016 | 24.638 | 100.808 | 67.759 | 1.00 | 24.66 | 7 |
| ATOM | 16850 | CA | TYR | G | 1016 | 25.120 | 99.813 | 66.812 | 1.00 | 22.45 | 6 |
| ATOM | 16851 | CB | TYR | G | 1016 | 25.221 | 100.412 | 65.409 | 1.00 | 21.71 | 6 |
| ATOM | 16852 | CG | TYR | G | 1016 | 24.070 | 101.305 | 65.057 | 1.00 | 19.54 | 6 |
| ATOM | 16853 | CD1 | TYR | G | 1016 | 22.787 | 100.970 | 65.437 | 1.00 | 19.92 | 6 |
| ATOM | 16854 | CE1 | TYR | G | 1016 | 21.715 | 101.769 | 65.119 | 1.00 | 20.03 | 6 |
| ATOM | 16855 | CD2 | TYR | G | 1016 | 24.259 | 102.483 | 64.344 | 1.00 | 18.54 | 6 |
| ATOM | 16856 | CE2 | TYR | G | 1016 | 23.189 | 103.293 | 64.015 | 1.00 | 19.28 | 6 |
| ATOM | 16857 | CZ | TYR | G | 1016 | 21.915 | 102.919 | 64.404 | 1.00 | 19.67 | 6 |
| ATOM | 16858 | OH | TYR | G | 1016 | 20.798 | 103.626 | 64.029 | 1.00 | 20.78 | 8 |
| ATOM | 16859 | C | TYR | G | 1016 | 26.479 | 99.227 | 67.194 | 1.00 | 23.39 | 6 |
| ATOM | 16860 | O | TYR | G | 1016 | 27.407 | 99.248 | 66.385 | 1.00 | 22.77 | 8 |
| ATOM | 16861 | N | PRO | G | 1017 | 26.624 | 98.695 | 68.426 | 1.00 | 56.70 | 7 |
| ATOM | 16862 | CD | PRO | G | 1017 | 25.624 | 98.598 | 69.501 | 1.00 | 33.23 | 6 |
| ATOM | 16863 | CA | PRO | G | 1017 | 27.898 | 98.106 | 68.859 | 1.00 | 57.58 | 6 |
| ATOM | 16864 | CB | PRO | G | 1017 | 27.646 | 97.786 | 70.330 | 1.00 | 32.92 | 6 |
| ATOM | 16865 | CG | PRO | G | 1017 | 26.187 | 97.488 | 70.353 | 1.00 | 33.40 | 6 |
| ATOM | 16866 | C | PRO | G | 1017 | 28.274 | 96.863 | 68.022 | 1.00 | 58.06 | 6 |
| ATOM | 16867 | O | PRO | G | 1017 | 29.357 | 96.293 | 68.175 | 1.00 | 56.67 | 8 |
| ATOM | 16868 | N | PHE | G | 1018 | 27.352 | 96.449 | 67.155 | 1.00 | 32.01 | 7 |
| ATOM | 16869 | CA | PHE | G | 1018 | 27.564 | 95.337 | 66.240 | 1.00 | 32.64 | 6 |
| ATOM | 16870 | CB | PHE | G | 1018 | 26.736 | 94.116 | 66.605 | 1.00 | 27.34 | 6 |
| ATOM | 16871 | CG | PHE | G | 1018 | 27.030 | 93.555 | 67.935 | 1.00 | 24.96 | 6 |
| ATOM | 16872 | CD1 | PHE | G | 1018 | 26.578 | 94.183 | 69.078 | 1.00 | 23.88 | 6 |
| ATOM | 16873 | CD2 | PHE | G | 1018 | 27.739 | 92.373 | 68.052 | 1.00 | 24.97 | 6 |
| ATOM | 16874 | CE1 | PHE | G | 1018 | 26.826 | 93.640 | 70.335 | 1.00 | 24.18 | 6 |
| ATOM | 16875 | CE2 | PHE | G | 1018 | 27.995 | 91.820 | 69.295 | 1.00 | 25.84 | 6 |
| ATOM | 16876 | CZ | PHE | G | 1018 | 27.535 | 92.455 | 70.446 | 1.00 | 25.55 | 6 |
| ATOM | 16877 | C | PHE | G | 1018 | 27.030 | 95.832 | 64.915 | 1.00 | 34.98 | 6 |
| ATOM | 16878 | O | PHE | G | 1018 | 27.758 | 95.999 | 63.936 | 1.00 | 33.54 | 8 |
| ATOM | 16879 | N | ASN | G | 1019 | 25.722 | 96.057 | 64.929 | 1.00 | 207.79 | 7 |
| ATOM | 16880 | CA | ASN | G | 1019 | 24.963 | 96.515 | 63.783 | 1.00 | 208.87 | 6 |
| ATOM | 16881 | CB | ASN | G | 1019 | 24.022 | 97.629 | 64.201 | 1.00 | 141.66 | 6 |
| ATOM | 16882 | CG | ASN | G | 1019 | 22.795 | 97.093 | 64.864 | 1.00 | 145.22 | 6 |
| ATOM | 16883 | OD1 | ASN | G | 1019 | 22.058 | 96.318 | 64.259 | 1.00 | 145.63 | 8 |
| ATOM | 16884 | ND2 | ASN | G | 1019 | 22.569 | 97.478 | 66.112 | 1.00 | 147.27 | 7 |
| ATOM | 16885 | C | ASN | G | 1019 | 25.757 | 96.928 | 62.575 | 1.00 | 208.87 | 6 |
| ATOM | 16886 | O | ASN | G | 1019 | 25.538 | 96.396 | 61.485 | 1.00 | 208.87 | 8 |
| ATOM | 16887 | N | PRO | G | 1020 | 26.678 | 97.889 | 62.723 | 1.00 | 35.68 | 7 |
| ATOM | 16888 | CD | PRO | G | 1020 | 27.066 | 98.923 | 63.688 | 1.00 | 157.45 | 6 |
| ATOM | 16889 | CA | PRO | G | 1020 | 27.318 | 98.114 | 61.441 | 1.00 | 34.69 | 6 |
| ATOM | 16890 | CB | PRO | G | 1020 | 28.161 | 99.357 | 61.684 | 1.00 | 156.72 | 6 |
| ATOM | 16891 | CG | PRO | G | 1020 | 27.417 | 100.069 | 62.773 | 1.00 | 158.71 | 6 |
| ATOM | 16892 | C | PRO | G | 1020 | 28.137 | 96.863 | 61.226 | 1.00 | 32.82 | 6 |
| ATOM | 16893 | O | PRO | G | 1020 | 29.106 | 96.604 | 61.932 | 1.00 | 31.89 | 8 |
| ATOM | 16894 | N | LEU | G | 1021 | 27.704 | 96.051 | 60.281 | 1.00 | 42.00 | 7 |
| ATOM | 16895 | CA | LEU | G | 1021 | 28.432 | 94.858 | 60.000 | 1.00 | 41.98 | 6 |
| ATOM | 16896 | CB | LEU | G | 1021 | 27.552 | 93.915 | 59.200 | 1.00 | 99.92 | 6 |
| ATOM | 16897 | CG | LEU | G | 1021 | 26.277 | 93.613 | 60.004 | 1.00 | 103.21 | 6 |
| ATOM | 16898 | CD1 | LEU | G | 1021 | 25.310 | 92.740 | 59.213 | 1.00 | 104.59 | 6 |
| ATOM | 16899 | CD2 | LEU | G | 1021 | 26.671 | 92.936 | 61.317 | 1.00 | 103.00 | 6 |
| ATOM | 16900 | C | LEU | G | 1021 | 29.620 | 95.388 | 59.222 | 1.00 | 41.58 | 6 |
| ATOM | 16901 | O | LEU | G | 1021 | 30.272 | 94.671 | 58.456 | 1.00 | 42.23 | 8 |
| ATOM | 16902 | N | ALA | G | 1022 | 29.874 | 96.680 | 59.449 | 1.00 | 52.95 | 7 |
| ATOM | 16903 | CA | ALA | G | 1022 | 30.979 | 97.435 | 58.867 | 1.00 | 50.22 | 6 |
| ATOM | 16904 | CB | ALA | G | 1022 | 30.462 | 98.571 | 57.940 | 1.00 | 13.87 | 6 |
| ATOM | 16905 | C | ALA | G | 1022 | 31.759 | 98.022 | 60.043 | 1.00 | 49.94 | 6 |
| ATOM | 16906 | O | ALA | G | 1022 | 32.563 | 98.930 | 59.858 | 1.00 | 52.39 | 8 |
| ATOM | 16907 | N | VAL | G | 1023 | 31.517 | 97.504 | 61.251 | 1.00 | 42.90 | 7 |
| ATOM | 16908 | CA | VAL | G | 1023 | 32.216 | 97.986 | 62.449 | 1.00 | 40.86 | 6 |
| ATOM | 16909 | CB | VAL | G | 1023 | 31.247 | 98.611 | 63.494 | 1.00 | 13.87 | 6 |
| ATOM | 16910 | CG1 | VAL | G | 1023 | 31.973 | 98.885 | 64.810 | 1.00 | 13.87 | 6 |
| ATOM | 16911 | CG2 | VAL | G | 1023 | 30.732 | 99.904 | 62.986 | 1.00 | 13.87 | 6 |
| ATOM | 16912 | C | VAL | G | 1023 | 33.060 | 96.936 | 63.160 | 1.00 | 40.24 | 6 |
| ATOM | 16913 | O | VAL | G | 1023 | 34.204 | 97.219 | 63.503 | 1.00 | 39.61 | 8 |
| ATOM | 16914 | N | MET | G | 1024 | 32.518 | 95.743 | 63.399 | 1.00 | 35.27 | 7 |
| ATOM | 16915 | CA | MET | G | 1024 | 33.297 | 94.701 | 64.072 | 1.00 | 36.89 | 6 |
| ATOM | 16916 | CB | MET | G | 1024 | 32.588 | 93.348 | 64.009 | 1.00 | 70.11 | 6 |
| ATOM | 16917 | CG | MET | G | 1024 | 31.097 | 93.437 | 63.892 | 1.00 | 76.08 | 6 |
| ATOM | 16918 | SD | MET | G | 1024 | 30.439 | 94.780 | 64.866 | 1.00 | 82.04 | 16 |
| ATOM | 16919 | CE | MET | G | 1024 | 30.639 | 94.123 | 66.544 | 1.00 | 82.88 | 6 |
| ATOM | 16920 | C | MET | G | 1024 | 34.608 | 94.603 | 63.309 | 1.00 | 37.14 | 6 |
| ATOM | 16921 | O | MET | G | 1024 | 35.690 | 94.445 | 63.887 | 1.00 | 36.34 | 8 |
| ATOM | 16922 | N | ALA | G | 1025 | 34.479 | 94.689 | 61.989 | 1.00 | 64.65 | 7 |
| ATOM | 16923 | CA | ALA | G | 1025 | 35.616 | 94.657 | 61.096 | 1.00 | 62.51 | 6 |
| ATOM | 16924 | CB | ALA | G | 1025 | 35.148 | 94.774 | 59.661 | 1.00 | 22.04 | 6 |
| ATOM | 16925 | C | ALA | G | 1025 | 36.420 | 95.876 | 61.480 | 1.00 | 61.26 | 6 |
| ATOM | 16926 | O | ALA | G | 1025 | 37.446 | 95.774 | 62.147 | 1.00 | 61.20 | 8 |
| ATOM | 16927 | N | ALA | G | 1026 | 35.914 | 97.035 | 61.072 | 1.00 | 34.88 | 7 |
| ATOM | 16928 | CA | ALA | G | 1026 | 36.547 | 98.317 | 61.363 | 1.00 | 32.65 | 6 |
| ATOM | 16929 | CB | ALA | G | 1026 | 35.501 | 99.429 | 61.319 | 1.00 | 75.61 | 6 |
| ATOM | 16930 | C | ALA | G | 1026 | 37.247 | 98.313 | 62.714 | 1.00 | 30.71 | 6 |
| ATOM | 16931 | O | ALA | G | 1026 | 38.432 | 98.614 | 62.803 | 1.00 | 28.71 | 8 |
| ATOM | 16932 | N | SER | G | 1027 | 36.509 | 97.961 | 63.760 | 1.00 | 22.46 | 7 |
| ATOM | 16933 | CA | SER | G | 1027 | 37.064 | 97.937 | 65.103 | 1.00 | 22.87 | 6 |
| ATOM | 16934 | CB | SER | G | 1027 | 35.969 | 98.095 | 66.162 | 1.00 | 29.31 | 6 |
| ATOM | 16935 | OG | SER | G | 1027 | 35.643 | 99.462 | 66.383 | 1.00 | 32.08 | 8 |
| ATOM | 16936 | C | SER | G | 1027 | 37.869 | 96.701 | 65.436 | 1.00 | 22.83 | 6 |
| ATOM | 16937 | O | SER | G | 1027 | 37.844 | 96.251 | 66.577 | 1.00 | 22.96 | 8 |
| ATOM | 16938 | N | GLY | G | 1028 | 38.592 | 96.154 | 64.466 | 1.00 | 23.58 | 7 |
| ATOM | 16939 | CA | GLY | G | 1028 | 39.384 | 94.976 | 64.759 | 1.00 | 23.25 | 6 |
| ATOM | 16940 | C | GLY | G | 1028 | 38.840 | 93.702 | 64.152 | 1.00 | 23.22 | 6 |
| ATOM | 16941 | O | GLY | G | 1028 | 37.960 | 93.049 | 64.724 | 1.00 | 22.96 | 8 |
| ATOM | 16942 | N | ALA | G | 1029 | 39.388 | 93.362 | 62.984 | 1.00 | 43.48 | 7 |
| ATOM | 16943 | CA | ALA | G | 1029 | 39.008 | 92.182 | 62.203 | 1.00 | 42.94 | 6 |
| ATOM | 16944 | CB | ALA | G | 1029 | 37.492 | 92.038 | 62.162 | 1.00 | 114.37 | 6 |
| ATOM | 16945 | C | ALA | G | 1029 | 39.560 | 92.324 | 60.782 | 1.00 | 41.27 | 6 |
| ATOM | 16946 | O | ALA | G | 1029 | 40.702 | 91.963 | 60.535 | 1.00 | 40.25 | 8 |
| ATOM | 16947 | N | ALA | G | 1030 | 38.743 | 92.851 | 59.864 | 1.00 | 18.27 | 7 |
| ATOM | 16948 | CA | ALA | G | 1030 | 39.154 | 93.059 | 58.471 | 1.00 | 17.47 | 6 |
| ATOM | 16949 | CB | ALA | G | 1030 | 38.833 | 91.817 | 57.668 | 1.00 | 13.87 | 6 |
| ATOM | 16950 | C | ALA | G | 1030 | 38.603 | 94.313 | 57.744 | 1.00 | 16.81 | 6 |
| ATOM | 16951 | O | ALA | G | 1030 | 38.171 | 94.216 | 56.602 | 1.00 | 14.21 | 8 |
| ATOM | 16952 | N | GLY | G | 1031 | 38.650 | 95.476 | 58.399 | 1.00 | 24.11 | 7 |
| ATOM | 16953 | CA | GLY | G | 1031 | 38.181 | 96.730 | 57.809 | 1.00 | 27.61 | 6 |
| ATOM | 16954 | C | GLY | G | 1031 | 39.156 | 97.870 | 58.097 | 1.00 | 28.66 | 6 |
| ATOM | 16955 | O | GLY | G | 1031 | 40.355 | 97.632 | 58.049 | 1.00 | 29.08 | 8 |
| ATOM | 16956 | N | ASN | G | 1032 | 38.671 | 99.086 | 58.394 | 1.00 | 31.32 | 7 |
| ATOM | 16957 | CA | ASN | G | 1032 | 39.537 | 100.253 | 58.714 | 1.00 | 32.32 | 6 |
| ATOM | 16958 | CB | ASN | G | 1032 | 40.711 | 100.293 | 57.742 | 1.00 | 40.11 | 6 |
| ATOM | 16959 | CG | ASN | G | 1032 | 40.749 | 101.546 | 56.925 | 1.00 | 41.52 | 6 |
| ATOM | 16960 | OD1 | ASN | G | 1032 | 40.997 | 102.629 | 57.457 | 1.00 | 40.09 | 8 |
| ATOM | 16961 | ND2 | ASN | G | 1032 | 40.507 | 101.415 | 55.615 | 1.00 | 41.76 | 7 |
| ATOM | 16962 | C | ASN | G | 1032 | 38.786 | 101.605 | 58.710 | 1.00 | 33.63 | 6 |
| ATOM | 16963 | O | ASN | G | 1032 | 37.928 | 101.827 | 57.860 | 1.00 | 33.89 | 8 |
| ATOM | 16964 | N | PRO | G | 1033 | 39.123 | 102.534 | 59.643 | 1.00 | 61.40 | 7 |
| ATOM | 16965 | CD | PRO | G | 1033 | 40.242 | 102.397 | 60.588 | 1.00 | 25.12 | 6 |
| ATOM | 16966 | CA | PRO | G | 1033 | 38.518 | 103.868 | 59.813 | 1.00 | 62.26 | 6 |
| ATOM | 16967 | CB | PRO | G | 1033 | 39.091 | 104.346 | 61.132 | 1.00 | 24.15 | 6 |
| ATOM | 16968 | CG | PRO | G | 1033 | 40.444 | 103.860 | 61.051 | 1.00 | 23.45 | 6 |
| ATOM | 16969 | C | PRO | G | 1033 | 38.693 | 104.930 | 58.758 | 1.00 | 64.25 | 6 |
| ATOM | 16970 | O | PRO | G | 1033 | 37.806 | 105.752 | 58.575 | 1.00 | 66.07 | 8 |
| ATOM | 16971 | N | GLN | G | 1034 | 39.836 | 104.965 | 58.093 | 1.00 | 53.14 | 7 |
| ATOM | 16972 | CA | GLN | G | 1034 | 40.013 | 105.982 | 57.065 | 1.00 | 54.20 | 6 |
| ATOM | 16973 | CB | GLN | G | 1034 | 41.437 | 105.968 | 56.514 | 1.00 | 98.46 | 6 |
| ATOM | 16974 | CG | GLN | G | 1034 | 41.839 | 104.665 | 55.872 | 1.00 | 102.25 | 6 |
| ATOM | 16975 | CD | GLN | G | 1034 | 42.929 | 104.849 | 54.848 | 1.00 | 104.72 | 6 |
| ATOM | 16976 | OE1 | GLN | G | 1034 | 43.940 | 105.501 | 55.118 | 1.00 | 107.00 | 8 |
| ATOM | 16977 | NE2 | GLN | G | 1034 | 42.736 | 104.274 | 53.660 | 1.00 | 105.14 | 7 |
| ATOM | 16978 | C | GLN | G | 1034 | 39.019 | 105.712 | 55.939 | 1.00 | 52.80 | 6 |
| ATOM | 16979 | O | GLN | G | 1034 | 38.436 | 106.628 | 55.359 | 1.00 | 53.41 | 8 |
| ATOM | 16980 | N | GLN | G | 1035 | 38.822 | 104.436 | 55.641 | 1.00 | 28.01 | 7 |
| ATOM | 16981 | CA | GLN | G | 1035 | 37.901 | 104.042 | 54.597 | 1.00 | 27.34 | 6 |
| ATOM | 16982 | CB | GLN | G | 1035 | 38.143 | 102.572 | 54.233 | 1.00 | 35.98 | 6 |
| ATOM | 16983 | CG | GLN | G | 1035 | 37.566 | 102.180 | 52.894 | 1.00 | 35.91 | 6 |
| ATOM | 16984 | CD | GLN | G | 1035 | 37.959 | 103.140 | 51.789 | 1.00 | 34.17 | 6 |
| ATOM | 16985 | OE1 | GLN | G | 1035 | 38.958 | 102.944 | 51.100 | 1.00 | 34.61 | 8 |
| ATOM | 16986 | NE2 | GLN | G | 1035 | 37.169 | 104.188 | 51.615 | 1.00 | 32.73 | 7 |
| ATOM | 16987 | C | GLN | G | 1035 | 36.471 | 104.291 | 55.096 | 1.00 | 27.53 | 6 |
| ATOM | 16988 | O | GLN | G | 1035 | 35.692 | 104.960 | 54.419 | 1.00 | 27.23 | 8 |
| ATOM | 16989 | N | ILE | G | 1036 | 36.139 | 103.771 | 56.283 | 1.00 | 38.83 | 7 |
| ATOM | 16990 | CA | ILE | G | 1036 | 34.817 | 103.973 | 56.893 | 1.00 | 38.39 | 6 |
| ATOM | 16991 | CB | ILE | G | 1036 | 34.842 | 103.670 | 58.403 | 1.00 | 29.15 | 6 |
| ATOM | 16992 | CG2 | ILE | G | 1036 | 33.456 | 103.848 | 58.996 | 1.00 | 29.71 | 6 |
| ATOM | 16993 | CG1 | ILE | G | 1036 | 35.325 | 102.245 | 58.645 | 1.00 | 29.83 | 6 |
| ATOM | 16994 | CD1 | ILE | G | 1036 | 34.278 | 101.197 | 58.439 | 1.00 | 33.08 | 6 |
| ATOM | 16995 | C | ILE | G | 1036 | 34.383 | 105.434 | 56.751 | 1.00 | 38.77 | 6 |
| ATOM | 16996 | O | ILE | G | 1036 | 33.326 | 105.728 | 56.194 | 1.00 | 38.44 | 8 |
| ATOM | 16997 | N | ARG | G | 1037 | 35.210 | 106.340 | 57.271 | 1.00 | 61.86 | 7 |
| ATOM | 16998 | CA | ARG | G | 1037 | 34.957 | 107.785 | 57.226 | 1.00 | 63.48 | 6 |
| ATOM | 16999 | CB | ARG | G | 1037 | 36.123 | 108.546 | 57.847 | 1.00 | 66.49 | 6 |
| ATOM | 17000 | CG | ARG | G | 1037 | 37.317 | 108.654 | 56.912 | 1.00 | 68.48 | 6 |
| ATOM | 17001 | CD | ARG | G | 1037 | 38.584 | 109.003 | 57.648 | 1.00 | 71.23 | 6 |
| ATOM | 17002 | NE | ARG | G | 1037 | 38.994 | 110.384 | 57.433 | 1.00 | 73.35 | 7 |
| ATOM | 17003 | CZ | ARG | G | 1037 | 40.004 | 110.959 | 58.079 | 1.00 | 74.16 | 6 |
| ATOM | 17004 | NH1 | ARG | G | 1037 | 40.697 | 110.264 | 58.976 | 1.00 | 75.40 | 7 |
| ATOM | 17005 | NH2 | ARG | G | 1037 | 40.322 | 112.224 | 57.839 | 1.00 | 73.81 | 7 |
| ATOM | 17006 | C | ARG | G | 1037 | 34.840 | 108.248 | 55.796 | 1.00 | 63.18 | 6 |
| ATOM | 17007 | O | ARG | G | 1037 | 34.764 | 109.441 | 55.526 | 1.00 | 64.48 | 8 |
| ATOM | 17008 | N | GLN | G | 1038 | 34.863 | 107.294 | 54.882 | 1.00 | 29.30 | 7 |
| ATOM | 17009 | CA | GLN | G | 1038 | 34.799 | 107.590 | 53.473 | 1.00 | 28.59 | 6 |
| ATOM | 17010 | CB | GLN | G | 1038 | 36.084 | 107.065 | 52.829 | 1.00 | 33.94 | 6 |
| ATOM | 17011 | CG | GLN | G | 1038 | 36.224 | 107.363 | 51.368 | 1.00 | 33.57 | 6 |
| ATOM | 17012 | CD | GLN | G | 1038 | 37.659 | 107.287 | 50.912 | 1.00 | 32.47 | 6 |
| ATOM | 17013 | OE1 | GLN | G | 1038 | 38.505 | 108.037 | 51.389 | 1.00 | 30.02 | 8 |
| ATOM | 17014 | NE2 | GLN | G | 1038 | 37.943 | 106.377 | 49.980 | 1.00 | 31.63 | 7 |
| ATOM | 17015 | C | GLN | G | 1038 | 33.541 | 106.963 | 52.868 | 1.00 | 28.87 | 6 |
| ATOM | 17016 | O | GLN | G | 1038 | 33.153 | 107.267 | 51.737 | 1.00 | 28.39 | 8 |
| ATOM | 17017 | N | LEU | G | 1039 | 32.899 | 106.103 | 53.654 | 1.00 | 70.71 | 7 |
| ATOM | 17018 | CA | LEU | G | 1039 | 31.682 | 105.415 | 53.242 | 1.00 | 70.80 | 6 |
| ATOM | 17019 | CB | LEU | G | 1039 | 31.738 | 103.936 | 53.676 | 1.00 | 42.09 | 6 |
| ATOM | 17020 | CG | LEU | G | 1039 | 32.453 | 102.888 | 52.796 | 1.00 | 40.18 | 6 |
| ATOM | 17021 | CD1 | LEU | G | 1039 | 33.221 | 101.911 | 53.676 | 1.00 | 39.29 | 6 |
| ATOM | 17022 | CD2 | LEU | G | 1039 | 31.435 | 102.134 | 51.934 | 1.00 | 39.21 | 6 |
| ATOM | 17023 | C | LEU | G | 1039 | 30.432 | 106.092 | 53.816 | 1.00 | 71.42 | 6 |
| ATOM | 17024 | O | LEU | G | 1039 | 29.384 | 106.097 | 53.173 | 1.00 | 71.93 | 8 |
| ATOM | 17025 | N | CYS | G | 1040 | 30.540 | 106.663 | 55.016 | 1.00 | 65.44 | 7 |
| ATOM | 17026 | CA | CYS | G | 1040 | 29.409 | 107.348 | 55.661 | 1.00 | 64.85 | 6 |
| ATOM | 17027 | CB | CYS | G | 1040 | 29.094 | 106.700 | 57.000 | 1.00 | 59.35 | 6 |
| ATOM | 17028 | SG | CYS | G | 1040 | 29.747 | 105.049 | 57.133 | 1.00 | 61.15 | 16 |
| ATOM | 17029 | C | CYS | G | 1040 | 29.771 | 108.804 | 55.904 | 1.00 | 63.95 | 6 |
| ATOM | 17030 | O | CYS | G | 1040 | 29.198 | 109.726 | 55.313 | 1.00 | 65.31 | 8 |
| ATOM | 17031 | N | GLY | G | 1041 | 30.714 | 109.005 | 56.809 | 1.00 | 55.48 | 7 |
| ATOM | 17032 | CA | GLY | G | 1041 | 31.161 | 110.350 | 57.080 | 1.00 | 53.62 | 6 |
| ATOM | 17033 | C | GLY | G | 1041 | 31.934 | 110.727 | 55.838 | 1.00 | 52.73 | 6 |
| ATOM | 17034 | O | GLY | G | 1041 | 32.603 | 109.885 | 55.239 | 1.00 | 53.70 | 8 |
| ATOM | 17035 | N | MET | G | 1042 | 31.836 | 111.989 | 55.449 | 1.00 | 25.63 | 7 |
| ATOM | 17036 | CA | MET | G | 1042 | 32.502 | 112.496 | 54.260 | 1.00 | 24.49 | 6 |
| ATOM | 17037 | CB | MET | G | 1042 | 32.111 | 113.972 | 54.092 | 1.00 | 25.46 | 6 |
| ATOM | 17038 | CG | MET | G | 1042 | 33.240 | 114.928 | 53.742 | 1.00 | 22.44 | 6 |
| ATOM | 17039 | SD | MET | G | 1042 | 34.025 | 115.738 | 55.131 | 1.00 | 19.24 | 16 |
| ATOM | 17040 | CE | MET | G | 1042 | 33.215 | 117.284 | 55.029 | 1.00 | 19.20 | 6 |
| ATOM | 17041 | C | MET | G | 1042 | 34.022 | 112.331 | 54.205 | 1.00 | 25.73 | 6 |
| ATOM | 17042 | O | MET | G | 1042 | 34.685 | 112.203 | 55.222 | 1.00 | 25.57 | 8 |
| ATOM | 17043 | N | ARG | G | 1043 | 34.547 | 112.292 | 52.988 | 1.00 | 44.89 | 7 |
| ATOM | 17044 | CA | ARG | G | 1043 | 35.986 | 112.233 | 52.715 | 1.00 | 48.27 | 6 |
| ATOM | 17045 | CB | ARG | G | 1043 | 36.429 | 110.873 | 52.194 | 1.00 | 65.34 | 6 |
| ATOM | 17046 | CG | ARG | G | 1043 | 35.770 | 110.465 | 50.882 | 1.00 | 65.04 | 6 |
| ATOM | 17047 | CD | ARG | G | 1043 | 36.784 | 110.183 | 49.795 | 1.00 | 65.29 | 6 |
| ATOM | 17048 | NE | ARG | G | 1043 | 36.243 | 109.239 | 48.826 | 1.00 | 65.65 | 7 |
| ATOM | 17049 | CZ | ARG | G | 1043 | 36.953 | 108.677 | 47.853 | 1.00 | 66.26 | 6 |
| ATOM | 17050 | NH1 | ARG | G | 1043 | 38.240 | 108.963 | 47.702 | 1.00 | 65.82 | 7 |
| ATOM | 17051 | NH2 | ARG | G | 1043 | 36.378 | 107.801 | 47.045 | 1.00 | 66.87 | 7 |
| ATOM | 17052 | C | ARG | G | 1043 | 35.984 | 113.219 | 51.571 | 1.00 | 50.08 | 6 |
| ATOM | 17053 | O | ARG | G | 1043 | 35.203 | 113.066 | 50.631 | 1.00 | 51.17 | 8 |
| ATOM | 17054 | N | GLY | G | 1044 | 36.813 | 114.248 | 51.627 | 1.00 | 57.92 | 7 |
| ATOM | 17055 | CA | GLY | G | 1044 | 36.731 | 115.192 | 50.540 | 1.00 | 59.57 | 6 |
| ATOM | 17056 | C | GLY | G | 1044 | 37.839 | 116.174 | 50.308 | 1.00 | 60.51 | 6 |
| ATOM | 17057 | O | GLY | G | 1044 | 37.808 | 116.869 | 49.296 | 1.00 | 61.34 | 8 |
| ATOM | 17058 | N | LEU | G | 1045 | 38.819 | 116.262 | 51.195 | 1.00 | 60.85 | 7 |
| ATOM | 17059 | CA | LEU | G | 1045 | 39.858 | 117.237 | 50.919 | 1.00 | 62.64 | 6 |
| ATOM | 17060 | CB | LEU | G | 1045 | 40.575 | 117.677 | 52.196 | 1.00 | 38.16 | 6 |
| ATOM | 17061 | CG | LEU | G | 1045 | 41.209 | 119.044 | 51.893 | 1.00 | 35.37 | 6 |
| ATOM | 17062 | CD1 | LEU | G | 1045 | 40.087 | 120.046 | 51.575 | 1.00 | 34.03 | 6 |
| ATOM | 17063 | CD2 | LEU | G | 1045 | 42.068 | 119.514 | 53.049 | 1.00 | 33.75 | 6 |
| ATOM | 17064 | C | LEU | G | 1045 | 40.865 | 116.725 | 49.888 | 1.00 | 64.76 | 6 |
| ATOM | 17065 | O | LEU | G | 1045 | 42.082 | 116.753 | 50.101 | 1.00 | 65.26 | 8 |
| ATOM | 17066 | N | MET | G | 1046 | 40.341 | 116.270 | 48.757 | 1.00 | 37.70 | 7 |
| ATOM | 17067 | CA | MET | G | 1046 | 41.166 | 115.757 | 47.684 | 1.00 | 40.48 | 6 |
| ATOM | 17068 | CB | MET | G | 1046 | 40.310 | 115.557 | 46.434 | 1.00 | 46.68 | 6 |
| ATOM | 17069 | CG | MET | G | 1046 | 40.245 | 114.123 | 45.958 | 1.00 | 46.40 | 6 |
| ATOM | 17070 | SD | MET | G | 1046 | 39.655 | 113.048 | 47.262 | 1.00 | 45.03 | 16 |
| ATOM | 17071 | CE | MET | G | 1046 | 41.205 | 112.756 | 48.251 | 1.00 | 44.35 | 6 |
| ATOM | 17072 | C | MET | G | 1046 | 42.307 | 116.716 | 47.383 | 1.00 | 43.07 | 6 |
| ATOM | 17073 | O | MET | G | 1046 | 42.280 | 117.873 | 47.802 | 1.00 | 43.33 | 8 |
| ATOM | 17074 | N | GLN | G | 1047 | 43.312 | 116.229 | 46.661 | 1.00 | 104.69 | 7 |
| ATOM | 17075 | CA | GLN | G | 1047 | 44.458 | 117.050 | 46.286 | 1.00 | 107.40 | 6 |
| ATOM | 17076 | CB | GLN | G | 1047 | 45.727 | 116.557 | 46.986 | 1.00 | 148.76 | 6 |
| ATOM | 17077 | CG | GLN | G | 1047 | 46.083 | 115.113 | 46.697 | 1.00 | 151.67 | 6 |
| ATOM | 17078 | CD | GLN | G | 1047 | 47.175 | 114.602 | 47.614 | 1.00 | 152.97 | 6 |
| ATOM | 17079 | OE1 | GLN | G | 1047 | 47.557 | 113.432 | 47.555 | 1.00 | 153.66 | 8 |
| ATOM | 17080 | NE2 | GLN | G | 1047 | 47.681 | 115.480 | 48.474 | 1.00 | 152.49 | 7 |
| ATOM | 17081 | C | GLN | G | 1047 | 44.646 | 117.011 | 44.777 | 1.00 | 107.91 | 6 |
| ATOM | 17082 | O | GLN | G | 1047 | 44.316 | 116.020 | 44.130 | 1.00 | 107.82 | 8 |
| ATOM | 17083 | N | LYS | G | 1048 | 45.162 | 118.100 | 44.219 | 1.00 | 73.19 | 7 |
| ATOM | 17084 | CA | LYS | G | 1048 | 45.385 | 118.180 | 42.787 | 1.00 | 74.01 | 6 |
| ATOM | 17085 | CB | LYS | G | 1048 | 45.861 | 119.584 | 42.398 | 1.00 | 114.73 | 6 |
| ATOM | 17086 | CG | LYS | G | 1048 | 44.784 | 120.651 | 42.584 | 1.00 | 116.62 | 6 |
| ATOM | 17087 | CD | LYS | G | 1048 | 45.227 | 122.030 | 42.112 | 1.00 | 118.68 | 6 |
| ATOM | 17088 | CE | LYS | G | 1048 | 44.177 | 123.084 | 42.454 | 1.00 | 119.59 | 6 |
| ATOM | 17089 | NZ | LYS | G | 1048 | 44.545 | 124.449 | 41.991 | 1.00 | 120.42 | 7 |
| ATOM | 17090 | C | LYS | G | 1048 | 46.401 | 117.129 | 42.398 | 1.00 | 73.88 | 6 |
| ATOM | 17091 | O | LYS | G | 1048 | 46.961 | 116.459 | 43.261 | 1.00 | 74.48 | 8 |
| ATOM | 17092 | N | PRO | G | 1049 | 46.636 | 116.947 | 41.093 | 1.00 | 68.45 | 7 |
| ATOM | 17093 | CD | PRO | G | 1049 | 46.020 | 117.628 | 39.942 | 1.00 | 55.63 | 6 |
| ATOM | 17094 | CA | PRO | G | 1049 | 47.604 | 115.948 | 40.646 | 1.00 | 68.78 | 6 |
| ATOM | 17095 | CB | PRO | G | 1049 | 47.338 | 115.873 | 39.154 | 1.00 | 55.58 | 6 |
| ATOM | 17096 | CG | PRO | G | 1049 | 46.963 | 117.285 | 38.828 | 1.00 | 55.59 | 6 |
| ATOM | 17097 | C | PRO | G | 1049 | 49.035 | 116.363 | 40.969 | 1.00 | 69.61 | 6 |
| ATOM | 17098 | O | PRO | G | 1049 | 49.964 | 115.578 | 40.804 | 1.00 | 70.14 | 8 |
| ATOM | 17099 | N | SER | G | 1050 | 49.197 | 117.598 | 41.438 | 1.00 | 81.57 | 7 |
| ATOM | 17100 | CA | SER | G | 1050 | 50.509 | 118.144 | 41.801 | 1.00 | 81.32 | 6 |
| ATOM | 17101 | CB | SER | G | 1050 | 50.787 | 119.411 | 41.003 | 1.00 | 56.87 | 6 |
| ATOM | 17102 | OG | SER | G | 1050 | 50.046 | 120.494 | 41.536 | 1.00 | 57.14 | 8 |
| ATOM | 17103 | C | SER | G | 1050 | 50.557 | 118.499 | 43.290 | 1.00 | 81.18 | 6 |
| ATOM | 17104 | O | SER | G | 1050 | 51.218 | 119.463 | 43.690 | 1.00 | 81.10 | 8 |
| ATOM | 17105 | N | GLY | G | 1051 | 49.847 | 117.720 | 44.102 | 1.00 | 113.08 | 7 |
| ATOM | 17106 | CA | GLY | G | 1051 | 49.803 | 117.973 | 45.531 | 1.00 | 111.73 | 6 |
| ATOM | 17107 | C | GLY | G | 1051 | 49.364 | 119.396 | 45.797 | 1.00 | 110.65 | 6 |
| ATOM | 17108 | O | GLY | G | 1051 | 50.143 | 120.205 | 46.288 | 1.00 | 111.24 | 8 |
| ATOM | 17109 | N | ALA | G | 1052 | 48.115 | 119.708 | 45.478 | 1.00 | 76.20 | 7 |
| ATOM | 17110 | CA | ALA | G | 1052 | 47.627 | 121.061 | 45.681 | 1.00 | 74.93 | 6 |
| ATOM | 17111 | CB | ALA | G | 1052 | 47.662 | 121.815 | 44.344 | 1.00 | 62.62 | 6 |
| ATOM | 17112 | C | ALA | G | 1052 | 46.227 | 121.142 | 46.305 | 1.00 | 74.29 | 6 |
| ATOM | 17113 | O | ALA | G | 1052 | 45.475 | 122.087 | 46.033 | 1.00 | 75.40 | 8 |
| ATOM | 17114 | N | THR | G | 1053 | 45.897 | 120.164 | 47.152 | 1.00 | 31.97 | 7 |
| ATOM | 17115 | CA | THR | G | 1053 | 44.591 | 120.091 | 47.818 | 1.00 | 30.20 | 6 |
| ATOM | 17116 | CB | THR | G | 1053 | 44.689 | 120.463 | 49.292 | 1.00 | 50.31 | 6 |
| ATOM | 17117 | OG1 | THR | G | 1053 | 45.958 | 121.082 | 49.535 | 1.00 | 52.45 | 8 |
| ATOM | 17118 | CG2 | THR | G | 1053 | 44.521 | 119.226 | 50.167 | 1.00 | 51.29 | 6 |
| ATOM | 17119 | C | THR | G | 1053 | 43.550 | 120.997 | 47.205 | 1.00 | 28.15 | 6 |
| ATOM | 17120 | O | THR | G | 1053 | 43.569 | 122.199 | 47.436 | 1.00 | 26.84 | 8 |
| ATOM | 17121 | N | PHE | G | 1054 | 42.647 | 120.433 | 46.416 | 1.00 | 59.95 | 7 |
| ATOM | 17122 | CA | PHE | G | 1054 | 41.624 | 121.257 | 45.812 | 1.00 | 60.48 | 6 |
| ATOM | 17123 | CB | PHE | G | 1054 | 40.641 | 120.410 | 45.025 | 1.00 | 66.54 | 6 |
| ATOM | 17124 | CG | PHE | G | 1054 | 41.227 | 119.790 | 43.796 | 1.00 | 67.65 | 6 |
| ATOM | 17125 | CD1 | PHE | G | 1054 | 41.975 | 118.622 | 43.877 | 1.00 | 67.50 | 6 |
| ATOM | 17126 | CD2 | PHE | G | 1054 | 41.010 | 120.363 | 42.547 | 1.00 | 67.81 | 6 |
| ATOM | 17127 | CE1 | PHE | G | 1054 | 42.491 | 118.033 | 42.733 | 1.00 | 67.85 | 6 |
| ATOM | 17128 | CE2 | PHE | G | 1054 | 41.527 | 119.780 | 41.395 | 1.00 | 67.89 | 6 |
| ATOM | 17129 | CZ | PHE | G | 1054 | 42.267 | 118.613 | 41.489 | 1.00 | 68.40 | 6 |
| ATOM | 17130 | C | PHE | G | 1054 | 40.896 | 122.008 | 46.918 | 1.00 | 60.72 | 6 |
| ATOM | 17131 | O | PHE | G | 1054 | 40.551 | 121.426 | 47.956 | 1.00 | 60.81 | 8 |
| ATOM | 17132 | N | GLU | G | 1055 | 40.673 | 123.301 | 46.680 | 1.00 | 49.92 | 7 |
| ATOM | 17133 | CA | GLU | G | 1055 | 40.016 | 124.199 | 47.623 | 1.00 | 48.19 | 6 |
| ATOM | 17134 | CB | GLU | G | 1055 | 40.000 | 125.604 | 47.024 | 1.00 | 56.84 | 6 |
| ATOM | 17135 | CG | GLU | G | 1055 | 39.805 | 126.741 | 48.014 | 1.00 | 58.12 | 6 |
| ATOM | 17136 | CD | GLU | G | 1055 | 40.276 | 128.083 | 47.457 | 1.00 | 59.13 | 6 |
| ATOM | 17137 | OE1 | GLU | G | 1055 | 40.417 | 128.200 | 46.222 | 1.00 | 60.11 | 8 |
| ATOM | 17138 | OE2 | GLU | G | 1055 | 40.500 | 129.026 | 48.248 | 1.00 | 60.04 | 8 |
| ATOM | 17139 | C | GLU | G | 1055 | 38.604 | 123.749 | 48.021 | 1.00 | 47.22 | 6 |
| ATOM | 17140 | O | GLU | G | 1055 | 38.124 | 124.124 | 49.085 | 1.00 | 47.12 | 8 |
| ATOM | 17141 | N | VAL | G | 1056 | 37.945 | 122.949 | 47.179 | 1.00 | 74.64 | 7 |
| ATOM | 17142 | CA | VAL | G | 1056 | 36.602 | 122.429 | 47.488 | 1.00 | 73.07 | 6 |
| ATOM | 17143 | CB | VAL | G | 1056 | 35.619 | 122.590 | 46.298 | 1.00 | 56.31 | 6 |
| ATOM | 17144 | CG1 | VAL | G | 1056 | 34.610 | 121.454 | 46.288 | 1.00 | 54.51 | 6 |
| ATOM | 17145 | CG2 | VAL | G | 1056 | 34.867 | 123.906 | 46.420 | 1.00 | 55.73 | 6 |
| ATOM | 17146 | C | VAL | G | 1056 | 36.670 | 120.944 | 47.844 | 1.00 | 72.41 | 6 |
| ATOM | 17147 | O | VAL | G | 1056 | 36.977 | 120.110 | 46.993 | 1.00 | 73.70 | 8 |
| ATOM | 17148 | N | PRO | G | 1057 | 36.370 | 120.593 | 49.107 | 1.00 | 23.65 | 7 |
| ATOM | 17149 | CD | PRO | G | 1057 | 35.806 | 121.415 | 50.190 | 1.00 | 61.10 | 6 |
| ATOM | 17150 | CA | PRO | G | 1057 | 36.418 | 119.194 | 49.519 | 1.00 | 22.52 | 6 |
| ATOM | 17151 | CB | PRO | G | 1057 | 36.225 | 119.283 | 51.026 | 1.00 | 59.37 | 6 |
| ATOM | 17152 | CG | PRO | G | 1057 | 35.239 | 120.362 | 51.133 | 1.00 | 59.92 | 6 |
| ATOM | 17153 | C | PRO | G | 1057 | 35.294 | 118.449 | 48.828 | 1.00 | 22.39 | 6 |
| ATOM | 17154 | O | PRO | G | 1057 | 34.716 | 118.960 | 47.862 | 1.00 | 22.06 | 8 |
| ATOM | 17155 | N | VAL | G | 1058 | 34.977 | 117.252 | 49.317 | 1.00 | 25.40 | 7 |
| ATOM | 17156 | CA | VAL | G | 1058 | 33.906 | 116.486 | 48.712 | 1.00 | 26.34 | 6 |
| ATOM | 17157 | CB | VAL | G | 1058 | 34.467 | 115.197 | 48.079 | 1.00 | 27.11 | 6 |
| ATOM | 17158 | CG1 | VAL | G | 1058 | 33.622 | 114.822 | 46.877 | 1.00 | 29.20 | 6 |
| ATOM | 17159 | CG2 | VAL | G | 1058 | 35.895 | 115.397 | 47.651 | 1.00 | 26.42 | 6 |
| ATOM | 17160 | C | VAL | G | 1058 | 32.747 | 116.126 | 49.664 | 1.00 | 27.04 | 6 |
| ATOM | 17161 | O | VAL | G | 1058 | 32.899 | 116.099 | 50.876 | 1.00 | 26.43 | 8 |
| ATOM | 17162 | N | ALA | G | 1059 | 31.574 | 115.884 | 49.097 | 1.00 | 71.48 | 7 |
| ATOM | 17163 | CA | ALA | G | 1059 | 30.418 | 115.473 | 49.883 | 1.00 | 73.02 | 6 |
| ATOM | 17164 | CB | ALA | G | 1059 | 29.192 | 116.248 | 49.456 | 1.00 | 172.76 | 6 |
| ATOM | 17165 | C | ALA | G | 1059 | 30.288 | 114.000 | 49.497 | 1.00 | 72.38 | 6 |
| ATOM | 17166 | O | ALA | G | 1059 | 29.463 | 113.621 | 48.646 | 1.00 | 71.49 | 8 |
| ATOM | 17167 | N | SER | G | 1060 | 31.120 | 113.174 | 50.126 | 1.00 | 42.81 | 7 |
| ATOM | 17168 | CA | SER | G | 1060 | 31.166 | 111.757 | 49.803 | 1.00 | 43.34 | 6 |
| ATOM | 17169 | CB | SER | G | 1060 | 32.612 | 111.249 | 49.901 | 1.00 | 151.82 | 6 |
| ATOM | 17170 | OG | SER | G | 1060 | 33.485 | 111.929 | 49.012 | 1.00 | 154.10 | 8 |
| ATOM | 17171 | C | SER | G | 1060 | 30.287 | 110.829 | 50.607 | 1.00 | 42.52 | 6 |
| ATOM | 17172 | O | SER | G | 1060 | 30.751 | 109.771 | 51.024 | 1.00 | 42.80 | 8 |
| ATOM | 17173 | N | SER | G | 1061 | 29.028 | 111.182 | 50.832 | 1.00 | 64.02 | 7 |
| ATOM | 17174 | CA | SER | G | 1061 | 28.185 | 110.274 | 51.598 | 1.00 | 64.44 | 6 |
| ATOM | 17175 | CB | SER | G | 1061 | 26.691 | 110.505 | 51.300 | 1.00 | 46.66 | 6 |
| ATOM | 17176 | OG | SER | G | 1061 | 26.195 | 111.687 | 51.908 | 1.00 | 45.87 | 8 |
| ATOM | 17177 | C | SER | G | 1061 | 28.603 | 108.862 | 51.166 | 1.00 | 65.14 | 6 |
| ATOM | 17178 | O | SER | G | 1061 | 28.620 | 107.944 | 51.985 | 1.00 | 65.32 | 8 |
| ATOM | 17179 | N | PHE | G | 1062 | 28.995 | 108.730 | 49.889 | 1.00 | 52.12 | 7 |
| ATOM | 17180 | CA | PHE | G | 1062 | 29.416 | 107.464 | 49.269 | 1.00 | 50.54 | 6 |
| ATOM | 17181 | CB | PHE | G | 1062 | 30.623 | 106.862 | 49.999 | 1.00 | 42.33 | 6 |
| ATOM | 17182 | CG | PHE | G | 1062 | 31.762 | 106.469 | 49.080 | 1.00 | 41.07 | 6 |
| ATOM | 17183 | CD1 | PHE | G | 1062 | 31.922 | 107.078 | 47.837 | 1.00 | 40.91 | 6 |
| ATOM | 17184 | CD2 | PHE | G | 1062 | 32.718 | 105.546 | 49.489 | 1.00 | 39.80 | 6 |
| ATOM | 17185 | CE1 | PHE | G | 1062 | 33.016 | 106.775 | 47.027 | 1.00 | 39.89 | 6 |
| ATOM | 17186 | CE2 | PHE | G | 1062 | 33.812 | 105.241 | 48.684 | 1.00 | 39.22 | 6 |
| ATOM | 17187 | CZ | PHE | G | 1062 | 33.961 | 105.855 | 47.458 | 1.00 | 39.25 | 6 |
| ATOM | 17188 | C | PHE | G | 1062 | 28.218 | 106.556 | 49.387 | 1.00 | 50.10 | 6 |
| ATOM | 17189 | O | PHE | G | 1062 | 27.690 | 106.061 | 48.392 | 1.00 | 48.76 | 8 |
| ATOM | 17190 | N | ARG | G | 1063 | 27.803 | 106.363 | 50.635 | 1.00 | 40.59 | 7 |
| ATOM | 17191 | CA | ARG | G | 1063 | 26.641 | 105.582 | 50.981 | 1.00 | 41.65 | 6 |
| ATOM | 17192 | CB | ARG | G | 1063 | 26.299 | 105.801 | 52.454 | 1.00 | 46.91 | 6 |
| ATOM | 17193 | CG | ARG | G | 1063 | 24.934 | 105.285 | 52.863 | 1.00 | 46.03 | 6 |
| ATOM | 17194 | CD | ARG | G | 1063 | 23.829 | 106.224 | 52.409 | 1.00 | 44.48 | 6 |
| ATOM | 17195 | NE | ARG | G | 1063 | 22.511 | 105.768 | 52.826 | 1.00 | 42.65 | 7 |
| ATOM | 17196 | CZ | ARG | G | 1063 | 22.218 | 105.351 | 54.056 | 1.00 | 42.52 | 6 |
| ATOM | 17197 | NH1 | ARG | G | 1063 | 23.158 | 105.326 | 54.993 | 1.00 | 40.99 | 7 |
| ATOM | 17198 | NH2 | ARG | G | 1063 | 20.975 | 104.979 | 54.361 | 1.00 | 42.77 | 7 |
| ATOM | 17199 | C | ARG | G | 1063 | 25.568 | 106.153 | 50.089 | 1.00 | 42.82 | 6 |
| ATOM | 17200 | O | ARG | G | 1063 | 24.900 | 105.440 | 49.359 | 1.00 | 42.84 | 8 |
| ATOM | 17201 | N | GLU | G | 1064 | 25.395 | 107.458 | 50.155 | 1.00 | 54.47 | 7 |
| ATOM | 17202 | CA | GLU | G | 1064 | 24.426 | 108.079 | 49.293 | 1.00 | 57.79 | 6 |
| ATOM | 17203 | CB | GLU | G | 1064 | 23.579 | 109.118 | 50.042 | 1.00 | 122.75 | 6 |
| ATOM | 17204 | CG | GLU | G | 1064 | 22.207 | 108.588 | 50.488 | 1.00 | 123.96 | 6 |
| ATOM | 17205 | CD | GLU | G | 1064 | 21.188 | 109.692 | 50.762 | 1.00 | 124.45 | 6 |
| ATOM | 17206 | OE1 | GLU | G | 1064 | 20.889 | 110.479 | 49.834 | 1.00 | 123.97 | 8 |
| ATOM | 17207 | OE2 | GLU | G | 1064 | 20.681 | 109.766 | 51.904 | 1.00 | 124.30 | 8 |
| ATOM | 17208 | C | GLU | G | 1064 | 25.221 | 108.727 | 48.184 | 1.00 | 58.91 | 6 |
| ATOM | 17209 | O | GLU | G | 1064 | 25.444 | 109.925 | 48.186 | 1.00 | 59.88 | 8 |
| ATOM | 17210 | N | GLY | G | 1065 | 25.678 | 107.899 | 47.256 | 1.00 | 134.40 | 7 |
| ATOM | 17211 | CA | GLY | G | 1065 | 26.430 | 108.376 | 46.112 | 1.00 | 135.02 | 6 |
| ATOM | 17212 | C | GLY | G | 1065 | 27.599 | 109.307 | 46.334 | 1.00 | 134.71 | 6 |
| ATOM | 17213 | O | GLY | G | 1065 | 27.993 | 109.606 | 47.461 | 1.00 | 135.65 | 8 |
| ATOM | 17214 | N | LEU | G | 1066 | 28.155 | 109.753 | 45.214 | 1.00 | 16.41 | 7 |
| ATOM | 17215 | CA | LEU | G | 1066 | 29.276 | 110.665 | 45.187 | 1.00 | 14.33 | 6 |
| ATOM | 17216 | CB | LEU | G | 1066 | 30.582 | 109.911 | 45.043 | 1.00 | 16.81 | 6 |
| ATOM | 17217 | CG | LEU | G | 1066 | 31.847 | 110.745 | 45.107 | 1.00 | 13.87 | 6 |
| ATOM | 17218 | CD1 | LEU | G | 1066 | 31.761 | 111.586 | 46.317 | 1.00 | 13.87 | 6 |
| ATOM | 17219 | CD2 | LEU | G | 1066 | 33.064 | 109.868 | 45.196 | 1.00 | 13.87 | 6 |
| ATOM | 17220 | C | LEU | G | 1066 | 28.988 | 111.375 | 43.920 | 1.00 | 15.38 | 6 |
| ATOM | 17221 | O | LEU | G | 1066 | 29.885 | 111.650 | 43.142 | 1.00 | 15.39 | 8 |
| ATOM | 17222 | N | THR | G | 1067 | 27.698 | 111.622 | 43.709 | 1.00 | 87.90 | 7 |
| ATOM | 17223 | CA | THR | G | 1067 | 27.183 | 112.308 | 42.528 | 1.00 | 90.97 | 6 |
| ATOM | 17224 | CB | THR | G | 1067 | 25.663 | 112.595 | 42.682 | 1.00 | 140.13 | 6 |
| ATOM | 17225 | OG1 | THR | G | 1067 | 24.988 | 111.415 | 43.141 | 1.00 | 140.49 | 8 |
| ATOM | 17226 | CG2 | THR | G | 1067 | 25.064 | 113.021 | 41.351 | 1.00 | 141.43 | 6 |
| ATOM | 17227 | C | THR | G | 1067 | 27.928 | 113.632 | 42.345 | 1.00 | 91.33 | 6 |
| ATOM | 17228 | O | THR | G | 1067 | 27.795 | 114.302 | 41.316 | 1.00 | 91.60 | 8 |
| ATOM | 17229 | N | VAL | G | 1068 | 28.707 | 113.993 | 43.363 | 1.00 | 81.99 | 7 |
| ATOM | 17230 | CA | VAL | G | 1068 | 29.496 | 115.213 | 43.360 | 1.00 | 81.36 | 6 |
| ATOM | 17231 | CB | VAL | G | 1068 | 30.665 | 115.128 | 44.405 | 1.00 | 87.41 | 6 |
| ATOM | 17232 | CG1 | VAL | G | 1068 | 31.290 | 116.499 | 44.622 | 1.00 | 88.37 | 6 |
| ATOM | 17233 | CG2 | VAL | G | 1068 | 30.150 | 114.580 | 45.737 | 1.00 | 87.01 | 6 |
| ATOM | 17234 | C | VAL | G | 1068 | 30.066 | 115.355 | 41.957 | 1.00 | 80.45 | 6 |
| ATOM | 17235 | O | VAL | G | 1068 | 30.207 | 116.460 | 41.451 | 1.00 | 81.03 | 8 |
| ATOM | 17236 | N | LEU | G | 1069 | 30.352 | 114.218 | 41.325 | 1.00 | 52.69 | 7 |
| ATOM | 17237 | CA | LEU | G | 1069 | 30.922 | 114.168 | 39.977 | 1.00 | 51.59 | 6 |
| ATOM | 17238 | CB | LEU | G | 1069 | 29.919 | 114.635 | 38.921 | 1.00 | 37.44 | 6 |
| ATOM | 17239 | CG | LEU | G | 1069 | 29.947 | 113.859 | 37.600 | 1.00 | 36.50 | 6 |
| ATOM | 17240 | CD1 | LEU | G | 1069 | 29.130 | 114.628 | 36.582 | 1.00 | 36.78 | 6 |
| ATOM | 17241 | CD2 | LEU | G | 1069 | 31.371 | 113.669 | 37.099 | 1.00 | 35.23 | 6 |
| ATOM | 17242 | C | LEU | G | 1069 | 32.175 | 115.034 | 39.936 | 1.00 | 51.26 | 6 |
| ATOM | 17243 | O | LEU | G | 1069 | 32.662 | 115.443 | 38.876 | 1.00 | 50.85 | 8 |
| ATOM | 17244 | N | GLU | G | 1070 | 32.674 | 115.325 | 41.129 | 1.00 | 77.89 | 7 |
| ATOM | 17245 | CA | GLU | G | 1070 | 33.901 | 116.072 | 41.278 | 1.00 | 77.46 | 6 |
| ATOM | 17246 | CB | GLU | G | 1070 | 33.995 | 116.663 | 42.687 | 1.00 | 149.79 | 6 |
| ATOM | 17247 | CG | GLU | G | 1070 | 34.944 | 117.852 | 42.834 | 1.00 | 154.08 | 6 |
| ATOM | 17248 | CD | GLU | G | 1070 | 34.408 | 119.136 | 42.207 | 1.00 | 156.34 | 6 |
| ATOM | 17249 | OE1 | GLU | G | 1070 | 33.355 | 119.643 | 42.655 | 1.00 | 157.77 | 8 |
| ATOM | 17250 | OE2 | GLU | G | 1070 | 35.044 | 119.642 | 41.263 | 1.00 | 157.61 | 8 |
| ATOM | 17251 | C | GLU | G | 1070 | 34.837 | 114.885 | 41.126 | 1.00 | 75.60 | 6 |
| ATOM | 17252 | O | GLU | G | 1070 | 36.051 | 115.038 | 41.034 | 1.00 | 75.21 | 8 |
| ATOM | 17253 | N | TYR | G | 1071 | 34.209 | 113.699 | 41.108 | 1.00 | 107.97 | 7 |
| ATOM | 17254 | CA | TYR | G | 1071 | 34.833 | 112.373 | 40.948 | 1.00 | 104.08 | 6 |
| ATOM | 17255 | CB | TYR | G | 1071 | 33.936 | 111.493 | 40.055 | 1.00 | 35.29 | 6 |
| ATOM | 17256 | CG | TYR | G | 1071 | 34.095 | 109.984 | 40.181 | 1.00 | 29.68 | 6 |
| ATOM | 17257 | CD1 | TYR | G | 1071 | 35.074 | 109.294 | 39.484 | 1.00 | 27.41 | 6 |
| ATOM | 17258 | CE1 | TYR | G | 1071 | 35.180 | 107.922 | 39.587 | 1.00 | 27.10 | 6 |
| ATOM | 17259 | CD2 | TYR | G | 1071 | 33.232 | 109.255 | 40.986 | 1.00 | 28.79 | 6 |
| ATOM | 17260 | CE2 | TYR | G | 1071 | 33.329 | 107.897 | 41.100 | 1.00 | 27.77 | 6 |
| ATOM | 17261 | CZ | TYR | G | 1071 | 34.298 | 107.226 | 40.404 | 1.00 | 27.50 | 6 |
| ATOM | 17262 | OH | TYR | G | 1071 | 34.366 | 105.852 | 40.545 | 1.00 | 26.32 | 8 |
| ATOM | 17263 | C | TYR | G | 1071 | 36.168 | 112.562 | 40.275 | 1.00 | 103.87 | 6 |
| ATOM | 17264 | O | TYR | G | 1071 | 37.174 | 111.960 | 40.652 | 1.00 | 104.64 | 8 |
| ATOM | 17265 | N | PHE | G | 1072 | 36.150 | 113.414 | 39.260 | 1.00 | 38.33 | 7 |
| ATOM | 17266 | CA | PHE | G | 1072 | 37.340 | 113.719 | 38.516 | 1.00 | 36.88 | 6 |
| ATOM | 17267 | CB | PHE | G | 1072 | 37.058 | 114.821 | 37.511 | 1.00 | 29.37 | 6 |
| ATOM | 17268 | CG | PHE | G | 1072 | 38.220 | 115.127 | 36.659 | 1.00 | 27.82 | 6 |
| ATOM | 17269 | CD1 | PHE | G | 1072 | 38.427 | 114.428 | 35.496 | 1.00 | 27.84 | 6 |
| ATOM | 17270 | CD2 | PHE | G | 1072 | 39.175 | 116.032 | 37.080 | 1.00 | 26.66 | 6 |
| ATOM | 17271 | CE1 | PHE | G | 1072 | 39.573 | 114.618 | 34.765 | 1.00 | 27.73 | 6 |
| ATOM | 17272 | CE2 | PHE | G | 1072 | 40.317 | 116.224 | 36.356 | 1.00 | 26.85 | 6 |
| ATOM | 17273 | CZ | PHE | G | 1072 | 40.522 | 115.511 | 35.191 | 1.00 | 27.27 | 6 |
| ATOM | 17274 | C | PHE | G | 1072 | 38.449 | 114.155 | 39.470 | 1.00 | 36.89 | 6 |
| ATOM | 17275 | O | PHE | G | 1072 | 39.519 | 113.561 | 39.494 | 1.00 | 37.20 | 8 |
| ATOM | 17276 | N | ILE | G | 1073 | 38.192 | 115.200 | 40.248 | 1.00 | 64.76 | 7 |
| ATOM | 17277 | CA | ILE | G | 1073 | 39.165 | 115.696 | 41.209 | 1.00 | 64.05 | 6 |
| ATOM | 17278 | CB | ILE | G | 1073 | 38.472 | 116.503 | 42.325 | 1.00 | 36.18 | 6 |
| ATOM | 17279 | CG2 | ILE | G | 1073 | 39.325 | 116.567 | 43.548 | 1.00 | 34.52 | 6 |
| ATOM | 17280 | CG1 | ILE | G | 1073 | 38.207 | 117.916 | 41.848 | 1.00 | 36.83 | 6 |
| ATOM | 17281 | CD1 | ILE | G | 1073 | 37.294 | 117.968 | 40.660 | 1.00 | 40.65 | 6 |
| ATOM | 17282 | C | ILE | G | 1073 | 39.952 | 114.549 | 41.836 | 1.00 | 64.73 | 6 |
| ATOM | 17283 | O | ILE | G | 1073 | 41.183 | 114.555 | 41.812 | 1.00 | 65.48 | 8 |
| ATOM | 17284 | N | SER | G | 1074 | 39.241 | 113.563 | 42.385 | 1.00 | 44.20 | 7 |
| ATOM | 17285 | CA | SER | G | 1074 | 39.886 | 112.413 | 43.025 | 1.00 | 43.60 | 6 |
| ATOM | 17286 | CB | SER | G | 1074 | 38.945 | 111.771 | 44.044 | 1.00 | 51.58 | 6 |
| ATOM | 17287 | OG | SER | G | 1074 | 37.888 | 111.099 | 43.391 | 1.00 | 51.10 | 8 |
| ATOM | 17288 | C | SER | G | 1074 | 40.329 | 111.353 | 42.017 | 1.00 | 43.55 | 6 |
| ATOM | 17289 | O | SER | G | 1074 | 41.104 | 110.461 | 42.364 | 1.00 | 43.93 | 8 |
| ATOM | 17290 | N | SER | G | 1075 | 39.834 | 111.455 | 40.781 | 1.00 | 37.72 | 7 |
| ATOM | 17291 | CA | SER | G | 1075 | 40.189 | 110.513 | 39.718 | 1.00 | 37.29 | 6 |
| ATOM | 17292 | CB | SER | G | 1075 | 39.585 | 110.946 | 38.373 | 1.00 | 27.72 | 6 |
| ATOM | 17293 | OG | SER | G | 1075 | 40.526 | 111.642 | 37.574 | 1.00 | 26.95 | 8 |
| ATOM | 17294 | C | SER | G | 1075 | 41.708 | 110.472 | 39.615 | 1.00 | 37.29 | 6 |
| ATOM | 17295 | O | SER | G | 1075 | 42.300 | 109.477 | 39.193 | 1.00 | 37.86 | 8 |
| ATOM | 17296 | N | HIS | G | 1076 | 42.333 | 111.571 | 40.006 | 1.00 | 23.97 | 7 |
| ATOM | 17297 | CA | HIS | G | 1076 | 43.778 | 111.651 | 39.994 | 1.00 | 24.01 | 6 |
| ATOM | 17298 | CB | HIS | G | 1076 | 44.243 | 113.092 | 40.215 | 1.00 | 69.89 | 6 |
| ATOM | 17299 | CG | HIS | G | 1076 | 43.531 | 114.103 | 39.367 | 1.00 | 71.73 | 6 |
| ATOM | 17300 | CD2 | HIS | G | 1076 | 42.223 | 114.236 | 39.045 | 1.00 | 72.14 | 6 |
| ATOM | 17301 | ND1 | HIS | G | 1076 | 44.178 | 115.171 | 38.784 | 1.00 | 73.19 | 7 |
| ATOM | 17302 | CE1 | HIS | G | 1076 | 43.300 | 115.919 | 38.141 | 1.00 | 72.53 | 6 |
| ATOM | 17303 | NE2 | HIS | G | 1076 | 42.106 | 115.373 | 38.285 | 1.00 | 72.39 | 7 |
| ATOM | 17304 | C | HIS | G | 1076 | 44.227 | 110.775 | 41.155 | 1.00 | 22.98 | 6 |
| ATOM | 17305 | O | HIS | G | 1076 | 43.972 | 109.579 | 41.143 | 1.00 | 22.29 | 8 |
| ATOM | 17306 | N | GLY | G | 1077 | 44.877 | 111.379 | 42.146 | 1.00 | 29.05 | 7 |
| ATOM | 17307 | CA | GLY | G | 1077 | 45.345 | 110.665 | 43.325 | 1.00 | 29.29 | 6 |
| ATOM | 17308 | C | GLY | G | 1077 | 45.300 | 109.142 | 43.364 | 1.00 | 30.06 | 6 |
| ATOM | 17309 | O | GLY | G | 1077 | 46.336 | 108.489 | 43.563 | 1.00 | 30.33 | 8 |
| ATOM | 17310 | N | ALA | G | 1078 | 44.107 | 108.566 | 43.218 | 1.00 | 30.46 | 7 |
| ATOM | 17311 | CA | ALA | G | 1078 | 43.969 | 107.113 | 43.221 | 1.00 | 31.01 | 6 |
| ATOM | 17312 | CB | ALA | G | 1078 | 42.550 | 106.717 | 42.849 | 1.00 | 92.82 | 6 |
| ATOM | 17313 | C | ALA | G | 1078 | 44.961 | 106.551 | 42.206 | 1.00 | 31.42 | 6 |
| ATOM | 17314 | O | ALA | G | 1078 | 45.534 | 105.488 | 42.408 | 1.00 | 31.49 | 8 |
| ATOM | 17315 | N | ARG | G | 1079 | 45.151 | 107.273 | 41.110 | 1.00 | 20.51 | 7 |
| ATOM | 17316 | CA | ARG | G | 1079 | 46.105 | 106.867 | 40.094 | 1.00 | 21.30 | 6 |
| ATOM | 17317 | CB | ARG | G | 1079 | 45.995 | 107.759 | 38.852 | 1.00 | 32.85 | 6 |
| ATOM | 17318 | CG | ARG | G | 1079 | 44.744 | 107.540 | 37.979 | 1.00 | 30.64 | 6 |
| ATOM | 17319 | CD | ARG | G | 1079 | 44.797 | 106.255 | 37.127 | 1.00 | 27.10 | 6 |
| ATOM | 17320 | NE | ARG | G | 1079 | 44.452 | 106.510 | 35.730 | 1.00 | 21.58 | 7 |
| ATOM | 17321 | CZ | ARG | G | 1079 | 45.223 | 107.188 | 34.897 | 1.00 | 19.55 | 6 |
| ATOM | 17322 | NH1 | ARG | G | 1079 | 46.374 | 107.670 | 35.318 | 1.00 | 20.65 | 7 |
| ATOM | 17323 | NH2 | ARG | G | 1079 | 44.846 | 107.388 | 33.654 | 1.00 | 15.90 | 7 |
| ATOM | 17324 | C | ARG | G | 1079 | 47.536 | 106.943 | 40.650 | 1.00 | 23.49 | 6 |
| ATOM | 17325 | O | ARG | G | 1079 | 48.287 | 105.982 | 40.543 | 1.00 | 23.18 | 8 |
| ATOM | 17326 | N | LYS | G | 1080 | 47.943 | 108.069 | 41.232 | 1.00 | 49.26 | 7 |
| ATOM | 17327 | CA | LYS | G | 1080 | 49.299 | 108.115 | 41.765 | 1.00 | 53.08 | 6 |
| ATOM | 17328 | CB | LYS | G | 1080 | 49.632 | 109.465 | 42.413 | 1.00 | 71.79 | 6 |
| ATOM | 17329 | CG | LYS | G | 1080 | 48.858 | 109.842 | 43.680 | 1.00 | 74.53 | 6 |
| ATOM | 17330 | CD | LYS | G | 1080 | 49.271 | 111.268 | 44.115 | 1.00 | 75.99 | 6 |
| ATOM | 17331 | CE | LYS | G | 1080 | 48.356 | 111.905 | 45.166 | 1.00 | 75.28 | 6 |
| ATOM | 17332 | NZ | LYS | G | 1080 | 48.592 | 113.384 | 45.230 | 1.00 | 73.92 | 7 |
| ATOM | 17333 | C | LYS | G | 1080 | 49.314 | 107.013 | 42.787 | 1.00 | 54.74 | 6 |
| ATOM | 17334 | O | LYS | G | 1080 | 50.326 | 106.352 | 42.997 | 1.00 | 55.03 | 8 |
| ATOM | 17335 | N | GLY | G | 1081 | 48.155 | 106.803 | 43.397 | 1.00 | 62.41 | 7 |
| ATOM | 17336 | CA | GLY | G | 1081 | 48.013 | 105.747 | 44.376 | 1.00 | 65.40 | 6 |
| ATOM | 17337 | C | GLY | G | 1081 | 47.994 | 104.403 | 43.665 | 1.00 | 66.82 | 6 |
| ATOM | 17338 | O | GLY | G | 1081 | 47.412 | 103.428 | 44.148 | 1.00 | 66.69 | 8 |
| ATOM | 17339 | N | GLY | G | 1082 | 48.637 | 104.361 | 42.504 | 1.00 | 41.64 | 7 |
| ATOM | 17340 | CA | GLY | G | 1082 | 48.697 | 103.147 | 41.711 | 1.00 | 43.79 | 6 |
| ATOM | 17341 | C | GLY | G | 1082 | 49.880 | 103.285 | 40.782 | 1.00 | 45.02 | 6 |
| ATOM | 17342 | O | GLY | G | 1082 | 50.269 | 102.347 | 40.088 | 1.00 | 43.67 | 8 |
| ATOM | 17343 | N | ALA | G | 1083 | 50.438 | 104.489 | 40.764 | 1.00 | 69.21 | 7 |
| ATOM | 17344 | CA | ALA | G | 1083 | 51.602 | 104.776 | 39.952 | 1.00 | 71.61 | 6 |
| ATOM | 17345 | CB | ALA | G | 1083 | 51.891 | 106.262 | 39.965 | 1.00 | 97.90 | 6 |
| ATOM | 17346 | C | ALA | G | 1083 | 52.720 | 104.015 | 40.641 | 1.00 | 73.49 | 6 |
| ATOM | 17347 | O | ALA | G | 1083 | 53.598 | 103.446 | 39.995 | 1.00 | 74.35 | 8 |
| ATOM | 17348 | N | ASP | G | 1084 | 52.671 | 104.006 | 41.970 | 1.00 | 68.22 | 7 |
| ATOM | 17349 | CA | ASP | G | 1084 | 53.671 | 103.300 | 42.757 | 1.00 | 70.05 | 6 |
| ATOM | 17350 | CB | ASP | G | 1084 | 53.648 | 103.764 | 44.224 | 1.00 | 137.38 | 6 |
| ATOM | 17351 | CG | ASP | G | 1084 | 54.382 | 105.092 | 44.438 | 1.00 | 138.90 | 6 |
| ATOM | 17352 | OD1 | ASP | G | 1084 | 55.593 | 105.179 | 44.131 | 1.00 | 138.95 | 8 |
| ATOM | 17353 | OD2 | ASP | G | 1084 | 53.746 | 106.051 | 44.923 | 1.00 | 140.40 | 8 |
| ATOM | 17354 | C | ASP | G | 1084 | 53.432 | 101.796 | 42.662 | 1.00 | 70.56 | 6 |
| ATOM | 17355 | O | ASP | G | 1084 | 54.378 | 101.027 | 42.549 | 1.00 | 70.04 | 8 |
| ATOM | 17356 | N | THR | G | 1085 | 52.173 | 101.367 | 42.691 | 1.00 | 74.52 | 7 |
| ATOM | 17357 | CA | THR | G | 1085 | 51.891 | 99.938 | 42.580 | 1.00 | 75.50 | 6 |
| ATOM | 17358 | CB | THR | G | 1085 | 50.377 | 99.657 | 42.478 | 1.00 | 68.09 | 6 |
| ATOM | 17359 | OG1 | THR | G | 1085 | 49.876 | 100.134 | 41.220 | 1.00 | 68.24 | 8 |
| ATOM | 17360 | CG2 | THR | G | 1085 | 49.642 | 100.342 | 43.619 | 1.00 | 68.36 | 6 |
| ATOM | 17361 | C | THR | G | 1085 | 52.588 | 99.415 | 41.324 | 1.00 | 76.40 | 6 |
| ATOM | 17362 | O | THR | G | 1085 | 52.778 | 98.211 | 41.148 | 1.00 | 76.52 | 8 |
| ATOM | 17363 | N | ALA | G | 1086 | 52.969 | 100.338 | 40.448 | 1.00 | 83.59 | 7 |
| ATOM | 17364 | CA | ALA | G | 1086 | 53.669 | 99.979 | 39.232 | 1.00 | 84.65 | 6 |
| ATOM | 17365 | CB | ALA | G | 1086 | 53.715 | 101.154 | 38.295 | 1.00 | 80.40 | 6 |
| ATOM | 17366 | C | ALA | G | 1086 | 55.073 | 99.591 | 39.664 | 1.00 | 85.51 | 6 |
| ATOM | 17367 | O | ALA | G | 1086 | 55.969 | 99.408 | 38.840 | 1.00 | 85.92 | 8 |
| ATOM | 17368 | N | LEU | G | 1087 | 55.240 | 99.482 | 40.980 | 1.00 | 102.04 | 7 |
| ATOM | 17369 | CA | LEU | G | 1087 | 56.494 | 99.093 | 41.622 | 1.00 | 103.66 | 6 |
| ATOM | 17370 | CB | LEU | G | 1087 | 56.471 | 99.525 | 43.089 | 1.00 | 121.59 | 6 |
| ATOM | 17371 | CG | LEU | G | 1087 | 57.702 | 99.415 | 43.984 | 1.00 | 122.48 | 6 |
| ATOM | 17372 | CD1 | LEU | G | 1087 | 58.753 | 100.432 | 43.564 | 1.00 | 122.43 | 6 |
| ATOM | 17373 | CD2 | LEU | G | 1087 | 57.275 | 99.674 | 45.417 | 1.00 | 121.96 | 6 |
| ATOM | 17374 | C | LEU | G | 1087 | 56.531 | 97.569 | 41.542 | 1.00 | 104.53 | 6 |
| ATOM | 17375 | O | LEU | G | 1087 | 57.367 | 96.907 | 42.160 | 1.00 | 105.07 | 8 |
| ATOM | 17376 | N | ARG | G | 1088 | 55.589 | 97.031 | 40.775 | 1.00 | 178.21 | 7 |
| ATOM | 17377 | CA | ARG | G | 1088 | 55.448 | 95.598 | 40.576 | 1.00 | 178.10 | 6 |
| ATOM | 17378 | CB | ARG | G | 1088 | 54.368 | 95.336 | 39.520 | 1.00 | 156.51 | 6 |
| ATOM | 17379 | CG | ARG | G | 1088 | 54.021 | 93.872 | 39.324 | 1.00 | 156.71 | 6 |
| ATOM | 17380 | CD | ARG | G | 1088 | 52.731 | 93.715 | 38.533 | 1.00 | 157.04 | 6 |
| ATOM | 17381 | NE | ARG | G | 1088 | 52.366 | 92.311 | 38.367 | 1.00 | 156.99 | 7 |
| ATOM | 17382 | CZ | ARG | G | 1088 | 51.218 | 91.893 | 37.845 | 1.00 | 156.86 | 6 |
| ATOM | 17383 | NH1 | ARG | G | 1088 | 50.312 | 92.770 | 37.436 | 1.00 | 156.51 | 7 |
| ATOM | 17384 | NH2 | ARG | G | 1088 | 50.976 | 90.594 | 37.726 | 1.00 | 156.68 | 7 |
| ATOM | 17385 | C | ARG | G | 1088 | 56.761 | 94.944 | 40.162 | 1.00 | 177.89 | 6 |
| ATOM | 17386 | O | ARG | G | 1088 | 57.267 | 94.063 | 40.861 | 1.00 | 178.39 | 8 |
| ATOM | 17387 | N | THR | G | 1089 | 57.313 | 95.376 | 39.031 | 1.00 | 70.41 | 7 |
| ATOM | 17388 | CA | THR | G | 1089 | 58.571 | 94.812 | 38.539 | 1.00 | 70.64 | 6 |
| ATOM | 17389 | CB | THR | G | 1089 | 58.841 | 95.209 | 37.070 | 1.00 | 84.81 | 6 |
| ATOM | 17390 | OG1 | THR | G | 1089 | 57.656 | 95.016 | 36.292 | 1.00 | 85.76 | 8 |
| ATOM | 17391 | CG2 | THR | G | 1089 | 59.947 | 94.351 | 36.486 | 1.00 | 84.90 | 6 |
| ATOM | 17392 | C | THR | G | 1089 | 59.719 | 95.335 | 39.393 | 1.00 | 70.04 | 6 |
| ATOM | 17393 | O | THR | G | 1089 | 60.761 | 94.686 | 39.522 | 1.00 | 69.06 | 8 |
| ATOM | 17394 | N | ALA | G | 1090 | 59.501 | 96.511 | 39.977 | 1.00 | 194.50 | 7 |
| ATOM | 17395 | CA | ALA | G | 1090 | 60.487 | 97.183 | 40.816 | 1.00 | 194.95 | 6 |
| ATOM | 17396 | CB | ALA | G | 1090 | 59.796 | 98.107 | 41.800 | 1.00 | 61.34 | 6 |
| ATOM | 17397 | C | ALA | G | 1090 | 61.404 | 96.240 | 41.569 | 1.00 | 195.05 | 6 |
| ATOM | 17398 | O | ALA | G | 1090 | 62.454 | 95.847 | 41.058 | 1.00 | 195.73 | 8 |
| ATOM | 17399 | N | ASP | G | 1091 | 61.003 | 95.872 | 42.783 | 1.00 | 93.23 | 7 |
| ATOM | 17400 | CA | ASP | G | 1091 | 61.828 | 94.998 | 43.605 | 1.00 | 92.34 | 6 |
| ATOM | 17401 | CB | ASP | G | 1091 | 61.231 | 94.889 | 45.013 | 1.00 | 83.99 | 6 |
| ATOM | 17402 | CG | ASP | G | 1091 | 61.340 | 96.202 | 45.789 | 1.00 | 83.05 | 6 |
| ATOM | 17403 | OD1 | ASP | G | 1091 | 62.478 | 96.654 | 46.062 | 1.00 | 82.49 | 8 |
| ATOM | 17404 | OD2 | ASP | G | 1091 | 60.285 | 96.786 | 46.115 | 1.00 | 82.36 | 8 |
| ATOM | 17405 | C | ASP | G | 1091 | 62.105 | 93.623 | 42.992 | 1.00 | 91.56 | 6 |
| ATOM | 17406 | O | ASP | G | 1091 | 62.663 | 92.731 | 43.645 | 1.00 | 91.17 | 8 |
| ATOM | 17407 | N | SER | G | 1092 | 61.725 | 93.470 | 41.726 | 1.00 | 129.54 | 7 |
| ATOM | 17408 | CA | SER | G | 1092 | 61.987 | 92.244 | 40.984 | 1.00 | 128.90 | 6 |
| ATOM | 17409 | CB | SER | G | 1092 | 60.798 | 91.888 | 40.085 | 1.00 | 194.76 | 6 |
| ATOM | 17410 | OG | SER | G | 1092 | 61.005 | 90.645 | 39.433 | 1.00 | 196.14 | 8 |
| ATOM | 17411 | C | SER | G | 1092 | 63.224 | 92.582 | 40.145 | 1.00 | 127.29 | 6 |
| ATOM | 17412 | O | SER | G | 1092 | 63.170 | 92.691 | 38.918 | 1.00 | 126.64 | 8 |
| ATOM | 17413 | N | GLY | G | 1093 | 64.336 | 92.772 | 40.845 | 1.00 | 143.66 | 7 |
| ATOM | 17414 | CA | GLY | G | 1093 | 65.589 | 93.125 | 40.206 | 1.00 | 140.25 | 6 |
| ATOM | 17415 | C | GLY | G | 1093 | 66.301 | 94.153 | 41.067 | 1.00 | 137.55 | 6 |
| ATOM | 17416 | O | GLY | G | 1093 | 67.356 | 94.670 | 40.698 | 1.00 | 137.13 | 8 |
| ATOM | 17417 | N | TYR | G | 1094 | 65.708 | 94.439 | 42.226 | 1.00 | 54.22 | 7 |
| ATOM | 17418 | CA | TYR | G | 1094 | 66.249 | 95.405 | 43.172 | 1.00 | 51.65 | 6 |
| ATOM | 17419 | CB | TYR | G | 1094 | 65.610 | 96.781 | 42.948 | 1.00 | 133.04 | 6 |
| ATOM | 17420 | CG | TYR | G | 1094 | 66.599 | 97.922 | 42.786 | 1.00 | 134.84 | 6 |
| ATOM | 17421 | CD1 | TYR | G | 1094 | 66.182 | 99.166 | 42.310 | 1.00 | 136.73 | 6 |
| ATOM | 17422 | CE1 | TYR | G | 1094 | 67.093 | 100.212 | 42.119 | 1.00 | 137.94 | 6 |
| ATOM | 17423 | CD2 | TYR | G | 1094 | 67.954 | 97.753 | 43.077 | 1.00 | 134.57 | 6 |
| ATOM | 17424 | CE2 | TYR | G | 1094 | 68.866 | 98.790 | 42.891 | 1.00 | 135.98 | 6 |
| ATOM | 17425 | CZ | TYR | G | 1094 | 68.434 | 100.014 | 42.410 | 1.00 | 137.81 | 6 |
| ATOM | 17426 | OH | TYR | G | 1094 | 69.346 | 101.027 | 42.203 | 1.00 | 138.02 | 8 |
| ATOM | 17427 | C | TYR | G | 1094 | 66.022 | 94.969 | 44.619 | 1.00 | 48.98 | 6 |
| ATOM | 17428 | O | TYR | G | 1094 | 65.163 | 94.136 | 44.922 | 1.00 | 47.60 | 8 |
| ATOM | 17429 | N | LEU | G | 1095 | 66.811 | 95.572 | 45.500 | 1.00 | 101.18 | 7 |
| ATOM | 17430 | CA | LEU | G | 1095 | 66.794 | 95.321 | 46.933 | 1.00 | 96.70 | 6 |
| ATOM | 17431 | CB | LEU | G | 1095 | 65.480 | 95.783 | 47.571 | 1.00 | 36.18 | 6 |
| ATOM | 17432 | CG | LEU | G | 1095 | 65.765 | 96.383 | 48.956 | 1.00 | 33.82 | 6 |
| ATOM | 17433 | CD1 | LEU | G | 1095 | 66.802 | 97.447 | 48.793 | 1.00 | 33.91 | 6 |
| ATOM | 17434 | CD2 | LEU | G | 1095 | 64.532 | 96.993 | 49.585 | 1.00 | 32.66 | 6 |
| ATOM | 17435 | C | LEU | G | 1095 | 67.076 | 93.874 | 47.283 | 1.00 | 94.62 | 6 |
| ATOM | 17436 | O | LEU | G | 1095 | 68.241 | 93.487 | 47.325 | 1.00 | 95.74 | 8 |
| ATOM | 17437 | N | THR | G | 1096 | 66.043 | 93.069 | 47.531 | 1.00 | 48.39 | 7 |
| ATOM | 17438 | CA | THR | G | 1096 | 66.287 | 91.671 | 47.886 | 1.00 | 44.22 | 6 |
| ATOM | 17439 | CB | THR | G | 1096 | 65.072 | 90.764 | 47.691 | 1.00 | 41.35 | 6 |
| ATOM | 17440 | OG1 | THR | G | 1096 | 64.002 | 91.198 | 48.527 | 1.00 | 42.13 | 8 |
| ATOM | 17441 | CG2 | THR | G | 1096 | 65.423 | 89.347 | 48.074 | 1.00 | 39.86 | 6 |
| ATOM | 17442 | C | THR | G | 1096 | 67.370 | 91.119 | 46.996 | 1.00 | 43.15 | 6 |
| ATOM | 17443 | O | THR | G | 1096 | 68.358 | 90.571 | 47.483 | 1.00 | 43.99 | 8 |
| ATOM | 17444 | N | ARG | G | 1097 | 67.184 | 91.271 | 45.687 | 1.00 | 29.03 | 7 |
| ATOM | 17445 | CA | ARG | G | 1097 | 68.159 | 90.766 | 44.732 | 1.00 | 25.02 | 6 |
| ATOM | 17446 | CB | ARG | G | 1097 | 67.828 | 91.212 | 43.310 | 1.00 | 28.45 | 6 |
| ATOM | 17447 | CG | ARG | G | 1097 | 68.784 | 90.655 | 42.277 | 1.00 | 23.84 | 6 |
| ATOM | 17448 | CD | ARG | G | 1097 | 68.499 | 91.168 | 40.869 | 1.00 | 19.46 | 6 |
| ATOM | 17449 | NE | ARG | G | 1097 | 67.263 | 90.635 | 40.304 | 1.00 | 16.77 | 7 |
| ATOM | 17450 | CZ | ARG | G | 1097 | 66.919 | 89.351 | 40.308 | 1.00 | 16.55 | 6 |
| ATOM | 17451 | NH1 | ARG | G | 1097 | 67.714 | 88.452 | 40.853 | 1.00 | 15.75 | 7 |
| ATOM | 17452 | NH2 | ARG | G | 1097 | 65.780 | 88.961 | 39.759 | 1.00 | 17.24 | 7 |
| ATOM | 17453 | C | ARG | G | 1097 | 69.519 | 91.279 | 45.124 | 1.00 | 24.71 | 6 |
| ATOM | 17454 | O | ARG | G | 1097 | 70.469 | 90.514 | 45.179 | 1.00 | 23.96 | 8 |
| ATOM | 17455 | N | LYS | G | 1098 | 69.598 | 92.572 | 45.426 | 1.00 | 46.29 | 7 |
| ATOM | 17456 | CA | LYS | G | 1098 | 70.860 | 93.191 | 45.814 | 1.00 | 47.25 | 6 |
| ATOM | 17457 | CB | LYS | G | 1098 | 70.722 | 94.709 | 45.744 | 1.00 | 73.47 | 6 |
| ATOM | 17458 | CG | LYS | G | 1098 | 70.735 | 95.226 | 44.313 | 1.00 | 77.29 | 6 |
| ATOM | 17459 | CD | LYS | G | 1098 | 70.969 | 96.726 | 44.262 | 1.00 | 80.57 | 6 |
| ATOM | 17460 | CE | LYS | G | 1098 | 71.525 | 97.160 | 42.908 | 1.00 | 82.23 | 6 |
| ATOM | 17461 | NZ | LYS | G | 1098 | 71.750 | 98.636 | 42.861 | 1.00 | 84.21 | 7 |
| ATOM | 17462 | C | LYS | G | 1098 | 71.426 | 92.760 | 47.174 | 1.00 | 45.94 | 6 |
| ATOM | 17463 | O | LYS | G | 1098 | 72.601 | 92.402 | 47.275 | 1.00 | 45.56 | 8 |
| ATOM | 17464 | N | LEU | G | 1099 | 70.601 | 92.800 | 48.213 | 1.00 | 15.40 | 7 |
| ATOM | 17465 | CA | LEU | G | 1099 | 71.036 | 92.388 | 49.534 | 1.00 | 13.87 | 6 |
| ATOM | 17466 | CB | LEU | G | 1099 | 69.944 | 92.639 | 50.582 | 1.00 | 18.91 | 6 |
| ATOM | 17467 | CG | LEU | G | 1099 | 69.566 | 94.053 | 51.039 | 1.00 | 17.91 | 6 |
| ATOM | 17468 | CD1 | LEU | G | 1099 | 68.710 | 94.779 | 50.010 | 1.00 | 17.09 | 6 |
| ATOM | 17469 | CD2 | LEU | G | 1099 | 68.801 | 93.919 | 52.332 | 1.00 | 17.63 | 6 |
| ATOM | 17470 | C | LEU | G | 1099 | 71.363 | 90.900 | 49.513 | 1.00 | 14.40 | 6 |
| ATOM | 17471 | O | LEU | G | 1099 | 72.361 | 90.476 | 50.091 | 1.00 | 15.05 | 8 |
| ATOM | 17472 | N | VAL | G | 1100 | 70.517 | 90.103 | 48.856 | 1.00 | 42.02 | 7 |
| ATOM | 17473 | CA | VAL | G | 1100 | 70.729 | 88.651 | 48.780 | 1.00 | 42.77 | 6 |
| ATOM | 17474 | CB | VAL | G | 1100 | 69.726 | 87.952 | 47.814 | 1.00 | 35.80 | 6 |
| ATOM | 17475 | CG1 | VAL | G | 1100 | 70.199 | 86.554 | 47.521 | 1.00 | 35.22 | 6 |
| ATOM | 17476 | CG2 | VAL | G | 1100 | 68.346 | 87.882 | 48.429 | 1.00 | 36.82 | 6 |
| ATOM | 17477 | C | VAL | G | 1100 | 72.134 | 88.335 | 48.298 | 1.00 | 43.84 | 6 |
| ATOM | 17478 | O | VAL | G | 1100 | 72.677 | 87.269 | 48.601 | 1.00 | 43.89 | 8 |
| ATOM | 17479 | N | ASP | G | 1101 | 72.707 | 89.267 | 47.539 | 1.00 | 44.54 | 7 |
| ATOM | 17480 | CA | ASP | G | 1101 | 74.048 | 89.115 | 46.999 | 1.00 | 44.01 | 6 |
| ATOM | 17481 | CB | ASP | G | 1101 | 74.198 | 89.914 | 45.707 | 1.00 | 34.48 | 6 |
| ATOM | 17482 | CG | ASP | G | 1101 | 73.492 | 89.251 | 44.533 | 1.00 | 35.27 | 6 |
| ATOM | 17483 | OD1 | ASP | G | 1101 | 73.459 | 87.999 | 44.524 | 1.00 | 33.92 | 8 |
| ATOM | 17484 | OD2 | ASP | G | 1101 | 72.986 | 89.958 | 43.619 | 1.00 | 35.56 | 8 |
| ATOM | 17485 | C | ASP | G | 1101 | 75.108 | 89.539 | 47.995 | 1.00 | 44.61 | 6 |
| ATOM | 17486 | O | ASP | G | 1101 | 76.209 | 88.999 | 47.992 | 1.00 | 45.63 | 8 |
| ATOM | 17487 | N | VAL | G | 1102 | 74.773 | 90.500 | 48.852 | 1.00 | 42.29 | 7 |
| ATOM | 17488 | CA | VAL | G | 1102 | 75.699 | 90.991 | 49.867 | 1.00 | 42.96 | 6 |
| ATOM | 17489 | CB | VAL | G | 1102 | 75.353 | 92.393 | 50.313 | 1.00 | 19.63 | 6 |
| ATOM | 17490 | CG1 | VAL | G | 1102 | 76.338 | 92.845 | 51.345 | 1.00 | 18.89 | 6 |
| ATOM | 17491 | CG2 | VAL | G | 1102 | 75.350 | 93.317 | 49.142 | 1.00 | 18.39 | 6 |
| ATOM | 17492 | C | VAL | G | 1102 | 75.661 | 90.123 | 51.103 | 1.00 | 44.74 | 6 |
| ATOM | 17493 | O | VAL | G | 1102 | 76.650 | 90.027 | 51.816 | 1.00 | 44.43 | 8 |
| ATOM | 17494 | N | ALA | G | 1103 | 74.503 | 89.528 | 51.372 | 1.00 | 54.22 | 7 |
| ATOM | 17495 | CA | ALA | G | 1103 | 74.335 | 88.646 | 52.515 | 1.00 | 56.42 | 6 |
| ATOM | 17496 | CB | ALA | G | 1103 | 72.882 | 88.605 | 52.934 | 1.00 | 99.83 | 6 |
| ATOM | 17497 | C | ALA | G | 1103 | 74.792 | 87.270 | 52.058 | 1.00 | 57.74 | 6 |
| ATOM | 17498 | O | ALA | G | 1103 | 75.655 | 87.172 | 51.197 | 1.00 | 58.10 | 8 |
| ATOM | 17499 | N | HIS | G | 1104 | 74.198 | 86.213 | 52.606 | 1.00 | 98.67 | 7 |
| ATOM | 17500 | CA | HIS | G | 1104 | 74.559 | 84.830 | 52.267 | 1.00 | 99.80 | 6 |
| ATOM | 17501 | CB | HIS | G | 1104 | 74.088 | 84.469 | 50.839 | 1.00 | 68.73 | 6 |
| ATOM | 17502 | CG | HIS | G | 1104 | 75.128 | 84.600 | 49.770 | 1.00 | 71.34 | 6 |
| ATOM | 17503 | CD2 | HIS | G | 1104 | 75.330 | 85.561 | 48.837 | 1.00 | 72.06 | 6 |
| ATOM | 17504 | ND1 | HIS | G | 1104 | 76.065 | 83.622 | 49.519 | 1.00 | 72.73 | 7 |
| ATOM | 17505 | CE1 | HIS | G | 1104 | 76.796 | 83.973 | 48.476 | 1.00 | 74.18 | 6 |
| ATOM | 17506 | NE2 | HIS | G | 1104 | 76.370 | 85.146 | 48.043 | 1.00 | 74.66 | 7 |
| ATOM | 17507 | C | HIS | G | 1104 | 76.056 | 84.662 | 52.472 | 1.00 | 99.23 | 6 |
| ATOM | 17508 | O | HIS | G | 1104 | 76.767 | 85.650 | 52.627 | 1.00 | 100.11 | 8 |
| ATOM | 17509 | N | GLU | G | 1105 | 76.549 | 83.430 | 52.504 | 1.00 | 48.15 | 7 |
| ATOM | 17510 | CA | GLU | G | 1105 | 77.969 | 83.234 | 52.787 | 1.00 | 47.26 | 6 |
| ATOM | 17511 | CB | GLU | G | 1105 | 78.838 | 84.099 | 51.874 | 1.00 | 66.71 | 6 |
| ATOM | 17512 | CG | GLU | G | 1105 | 80.222 | 84.396 | 52.415 | 1.00 | 67.87 | 6 |
| ATOM | 17513 | CD | GLU | G | 1105 | 80.555 | 85.873 | 52.341 | 1.00 | 68.89 | 6 |
| ATOM | 17514 | OE1 | GLU | G | 1105 | 79.711 | 86.691 | 52.770 | 1.00 | 67.97 | 8 |
| ATOM | 17515 | OE2 | GLU | G | 1105 | 81.661 | 86.214 | 51.862 | 1.00 | 70.37 | 8 |
| ATOM | 17516 | C | GLU | G | 1105 | 78.092 | 83.733 | 54.221 | 1.00 | 46.84 | 6 |
| ATOM | 17517 | O | GLU | G | 1105 | 79.159 | 83.714 | 54.827 | 1.00 | 48.11 | 8 |
| ATOM | 17518 | N | ILE | G | 1106 | 76.960 | 84.189 | 54.742 | 1.00 | 53.44 | 7 |
| ATOM | 17519 | CA | ILE | G | 1106 | 76.830 | 84.722 | 56.085 | 1.00 | 51.14 | 6 |
| ATOM | 17520 | CB | ILE | G | 1106 | 76.142 | 86.124 | 56.027 | 1.00 | 15.21 | 6 |
| ATOM | 17521 | CG2 | ILE | G | 1106 | 75.818 | 86.656 | 57.388 | 1.00 | 14.34 | 6 |
| ATOM | 17522 | CG1 | ILE | G | 1106 | 77.089 | 87.117 | 55.382 | 1.00 | 14.30 | 6 |
| ATOM | 17523 | CD1 | ILE | G | 1106 | 78.412 | 87.180 | 56.084 | 1.00 | 15.16 | 6 |
| ATOM | 17524 | C | ILE | G | 1106 | 75.950 | 83.714 | 56.793 | 1.00 | 50.82 | 6 |
| ATOM | 17525 | O | ILE | G | 1106 | 74.735 | 83.842 | 56.785 | 1.00 | 51.03 | 8 |
| ATOM | 17526 | N | VAL | G | 1107 | 76.559 | 82.696 | 57.387 | 1.00 | 17.44 | 7 |
| ATOM | 17527 | CA | VAL | G | 1107 | 75.782 | 81.665 | 58.071 | 1.00 | 17.20 | 6 |
| ATOM | 17528 | CB | VAL | G | 1107 | 75.922 | 80.319 | 57.335 | 1.00 | 19.16 | 6 |
| ATOM | 17529 | CG1 | VAL | G | 1107 | 75.114 | 79.254 | 58.039 | 1.00 | 20.01 | 6 |
| ATOM | 17530 | CG2 | VAL | G | 1107 | 75.473 | 80.470 | 55.893 | 1.00 | 17.33 | 6 |
| ATOM | 17531 | C | VAL | G | 1107 | 76.127 | 81.459 | 59.549 | 1.00 | 18.68 | 6 |
| ATOM | 17532 | O | VAL | G | 1107 | 77.233 | 81.724 | 59.980 | 1.00 | 19.66 | 8 |
| ATOM | 17533 | N | VAL | G | 1108 | 75.160 | 80.998 | 60.326 | 1.00 | 45.65 | 7 |
| ATOM | 17534 | CA | VAL | G | 1108 | 75.378 | 80.751 | 61.740 | 1.00 | 47.62 | 6 |
| ATOM | 17535 | CB | VAL | G | 1108 | 74.056 | 80.354 | 62.422 | 1.00 | 55.91 | 6 |
| ATOM | 17536 | CG1 | VAL | G | 1108 | 74.293 | 80.000 | 63.867 | 1.00 | 56.80 | 6 |
| ATOM | 17537 | CG2 | VAL | G | 1108 | 73.061 | 81.485 | 62.307 | 1.00 | 55.44 | 6 |
| ATOM | 17538 | C | VAL | G | 1108 | 76.378 | 79.607 | 61.848 | 1.00 | 49.77 | 6 |
| ATOM | 17539 | O | VAL | G | 1108 | 76.005 | 78.435 | 61.777 | 1.00 | 50.63 | 8 |
| ATOM | 17540 | N | ARG | G | 1109 | 77.652 | 79.947 | 62.006 | 1.00 | 53.06 | 7 |
| ATOM | 17541 | CA | ARG | G | 1109 | 78.701 | 78.936 | 62.106 | 1.00 | 53.93 | 6 |
| ATOM | 17542 | CB | ARG | G | 1109 | 79.727 | 79.139 | 60.994 | 1.00 | 54.75 | 6 |
| ATOM | 17543 | CG | ARG | G | 1109 | 79.117 | 79.547 | 59.665 | 1.00 | 54.83 | 6 |
| ATOM | 17544 | CD | ARG | G | 1109 | 80.168 | 79.715 | 58.586 | 1.00 | 55.76 | 6 |
| ATOM | 17545 | NE | ARG | G | 1109 | 79.608 | 80.345 | 57.398 | 1.00 | 58.64 | 7 |
| ATOM | 17546 | CZ | ARG | G | 1109 | 80.107 | 80.194 | 56.177 | 1.00 | 60.66 | 6 |
| ATOM | 17547 | NH1 | ARG | G | 1109 | 81.173 | 79.431 | 55.996 | 1.00 | 62.22 | 7 |
| ATOM | 17548 | NH2 | ARG | G | 1109 | 79.543 | 80.792 | 55.135 | 1.00 | 61.45 | 7 |
| ATOM | 17549 | C | ARG | G | 1109 | 79.397 | 79.064 | 63.447 | 1.00 | 55.14 | 6 |
| ATOM | 17550 | O | ARG | G | 1109 | 79.948 | 80.118 | 63.739 | 1.00 | 56.53 | 8 |
| ATOM | 17551 | N | GLU | G | 1110 | 79.354 | 77.997 | 64.248 | 1.00 | 69.74 | 7 |
| ATOM | 17552 | CA | GLU | G | 1110 | 79.978 | 77.929 | 65.580 | 1.00 | 70.44 | 6 |
| ATOM | 17553 | CB | GLU | G | 1110 | 81.361 | 78.608 | 65.580 | 1.00 | 29.53 | 6 |
| ATOM | 17554 | CG | GLU | G | 1110 | 81.372 | 80.083 | 65.958 | 1.00 | 27.59 | 6 |
| ATOM | 17555 | CD | GLU | G | 1110 | 82.728 | 80.726 | 65.753 | 1.00 | 27.62 | 6 |
| ATOM | 17556 | OE1 | GLU | G | 1110 | 83.703 | 80.239 | 66.369 | 1.00 | 28.62 | 8 |
| ATOM | 17557 | OE2 | GLU | G | 1110 | 82.816 | 81.713 | 64.978 | 1.00 | 26.76 | 8 |
| ATOM | 17558 | C | GLU | G | 1110 | 79.130 | 78.504 | 66.714 | 1.00 | 71.85 | 6 |
| ATOM | 17559 | O | GLU | G | 1110 | 77.916 | 78.575 | 66.624 | 1.00 | 72.65 | 8 |
| ATOM | 17560 | N | ALA | G | 1111 | 79.777 | 78.897 | 67.801 | 1.00 | 70.73 | 7 |
| ATOM | 17561 | CA | ALA | G | 1111 | 79.048 | 79.459 | 68.922 | 1.00 | 71.48 | 6 |
| ATOM | 17562 | CB | ALA | G | 1111 | 78.464 | 78.335 | 69.781 | 1.00 | 13.87 | 6 |
| ATOM | 17563 | C | ALA | G | 1111 | 79.922 | 80.388 | 69.769 | 1.00 | 73.10 | 6 |
| ATOM | 17564 | O | ALA | G | 1111 | 81.158 | 80.359 | 69.681 | 1.00 | 73.20 | 8 |
| ATOM | 17565 | N | ALA | G | 1112 | 79.247 | 81.215 | 70.571 | 1.00 | 149.19 | 7 |
| ATOM | 17566 | CA | ALA | G | 1112 | 79.857 | 82.195 | 71.475 | 1.00 | 150.93 | 6 |
| ATOM | 17567 | CB | ALA | G | 1112 | 79.488 | 81.860 | 72.920 | 1.00 | 126.92 | 6 |
| ATOM | 17568 | C | ALA | G | 1112 | 81.365 | 82.323 | 71.350 | 1.00 | 151.11 | 6 |
| ATOM | 17569 | O | ALA | G | 1112 | 82.088 | 82.123 | 72.324 | 1.00 | 151.26 | 8 |
| ATOM | 17570 | N | CYS | G | 1113 | 81.829 | 82.667 | 70.153 | 1.00 | 86.03 | 7 |
| ATOM | 17571 | CA | CYS | G | 1113 | 83.251 | 82.818 | 69.893 | 1.00 | 85.94 | 6 |
| ATOM | 17572 | CB | CYS | G | 1113 | 83.468 | 83.616 | 68.610 | 1.00 | 58.96 | 6 |
| ATOM | 17573 | SG | CYS | G | 1113 | 82.777 | 85.280 | 68.651 | 1.00 | 57.92 | 16 |
| ATOM | 17574 | C | CYS | G | 1113 | 83.944 | 83.523 | 71.047 | 1.00 | 86.24 | 6 |
| ATOM | 17575 | O | CYS | G | 1113 | 84.355 | 82.889 | 72.019 | 1.00 | 86.35 | 8 |
| ATOM | 17576 | N | GLY | G | 1114 | 84.073 | 84.839 | 70.934 | 1.00 | 68.89 | 7 |
| ATOM | 17577 | CA | GLY | G | 1114 | 84.715 | 85.615 | 71.976 | 1.00 | 69.12 | 6 |
| ATOM | 17578 | C | GLY | G | 1114 | 84.082 | 86.984 | 72.042 | 1.00 | 68.79 | 6 |
| ATOM | 17579 | O | GLY | G | 1114 | 84.363 | 87.840 | 71.204 | 1.00 | 68.51 | 8 |
| ATOM | 17580 | N | ALA | G | 1115 | 83.220 | 87.181 | 73.034 | 1.00 | 69.28 | 7 |
| ATOM | 17581 | CA | ALA | G | 1115 | 82.523 | 88.450 | 73.217 | 1.00 | 69.62 | 6 |
| ATOM | 17582 | CB | ALA | G | 1115 | 81.777 | 88.448 | 74.550 | 1.00 | 127.54 | 6 |
| ATOM | 17583 | C | ALA | G | 1115 | 83.490 | 89.626 | 73.164 | 1.00 | 69.24 | 6 |
| ATOM | 17584 | O | ALA | G | 1115 | 83.977 | 90.000 | 72.092 | 1.00 | 68.67 | 8 |
| ATOM | 17585 | N | ALA | G | 1116 | 83.759 | 90.214 | 74.322 | 1.00 | 92.92 | 7 |
| ATOM | 17586 | CA | ALA | G | 1116 | 84.674 | 91.333 | 74.382 | 1.00 | 92.82 | 6 |
| ATOM | 17587 | CB | ALA | G | 1116 | 86.066 | 90.880 | 73.961 | 1.00 | 98.99 | 6 |
| ATOM | 17588 | C | ALA | G | 1116 | 84.199 | 92.460 | 73.476 | 1.00 | 92.10 | 6 |
| ATOM | 17589 | O | ALA | G | 1116 | 84.972 | 93.359 | 73.142 | 1.00 | 93.50 | 8 |
| ATOM | 17590 | N | ALA | G | 1117 | 82.937 | 92.407 | 73.063 | 1.00 | 76.64 | 7 |
| ATOM | 17591 | CA | ALA | G | 1117 | 82.377 | 93.451 | 72.206 | 1.00 | 74.11 | 6 |
| ATOM | 17592 | CB | ALA | G | 1117 | 82.894 | 93.316 | 70.791 | 1.00 | 43.28 | 6 |
| ATOM | 17593 | C | ALA | G | 1117 | 80.856 | 93.404 | 72.227 | 1.00 | 72.27 | 6 |
| ATOM | 17594 | O | ALA | G | 1117 | 80.254 | 92.344 | 72.342 | 1.00 | 71.97 | 8 |
| ATOM | 17595 | N | ALA | G | 1118 | 80.238 | 94.568 | 72.106 | 1.00 | 31.47 | 7 |
| ATOM | 17596 | CA | ALA | G | 1118 | 78.801 | 94.670 | 72.167 | 1.00 | 28.93 | 6 |
| ATOM | 17597 | CB | ALA | G | 1118 | 78.383 | 94.776 | 73.618 | 1.00 | 13.87 | 6 |
| ATOM | 17598 | C | ALA | G | 1118 | 78.342 | 95.900 | 71.400 | 1.00 | 28.09 | 6 |
| ATOM | 17599 | O | ALA | G | 1118 | 79.118 | 96.528 | 70.689 | 1.00 | 27.74 | 8 |
| ATOM | 17600 | N | ILE | G | 1119 | 77.063 | 96.223 | 71.545 | 1.00 | 46.47 | 7 |
| ATOM | 17601 | CA | ILE | G | 1119 | 76.441 | 97.383 | 70.909 | 1.00 | 45.45 | 6 |
| ATOM | 17602 | CB | ILE | G | 1119 | 75.636 | 97.019 | 69.627 | 1.00 | 35.54 | 6 |
| ATOM | 17603 | CG2 | ILE | G | 1119 | 75.325 | 98.261 | 68.810 | 1.00 | 34.58 | 6 |
| ATOM | 17604 | CG1 | ILE | G | 1119 | 76.414 | 96.028 | 68.777 | 1.00 | 34.64 | 6 |
| ATOM | 17605 | CD1 | ILE | G | 1119 | 76.379 | 94.635 | 69.339 | 1.00 | 35.67 | 6 |
| ATOM | 17606 | C | ILE | G | 1119 | 75.425 | 97.711 | 71.972 | 1.00 | 45.61 | 6 |
| ATOM | 17607 | O | ILE | G | 1119 | 74.536 | 96.901 | 72.224 | 1.00 | 45.90 | 8 |
| ATOM | 17608 | N | SER | G | 1120 | 75.542 | 98.858 | 72.623 | 1.00 | 37.17 | 7 |
| ATOM | 17609 | CA | SER | G | 1120 | 74.560 | 99.159 | 73.645 | 1.00 | 36.77 | 6 |
| ATOM | 17610 | CB | SER | G | 1120 | 75.193 | 99.967 | 74.772 | 1.00 | 44.93 | 6 |
| ATOM | 17611 | OG | SER | G | 1120 | 75.383 | 99.135 | 75.910 | 1.00 | 46.11 | 8 |
| ATOM | 17612 | C | SER | G | 1120 | 73.344 | 99.871 | 73.066 | 1.00 | 36.37 | 6 |
| ATOM | 17613 | O | SER | G | 1120 | 73.306 | 101.097 | 72.985 | 1.00 | 37.49 | 8 |
| ATOM | 17614 | N | VAL | G | 1121 | 72.349 | 99.076 | 72.667 | 1.00 | 32.16 | 7 |
| ATOM | 17615 | CA | VAL | G | 1121 | 71.122 | 99.582 | 72.071 | 1.00 | 30.39 | 6 |
| ATOM | 17616 | CB | VAL | G | 1121 | 70.135 | 98.460 | 71.824 | 1.00 | 58.71 | 6 |
| ATOM | 17617 | CG1 | VAL | G | 1121 | 69.005 | 98.963 | 70.963 | 1.00 | 60.29 | 6 |
| ATOM | 17618 | CG2 | VAL | G | 1121 | 70.840 | 97.290 | 71.172 | 1.00 | 59.82 | 6 |
| ATOM | 17619 | C | VAL | G | 1121 | 70.464 | 100.589 | 72.980 | 1.00 | 29.18 | 6 |
| ATOM | 17620 | O | VAL | G | 1121 | 70.232 | 100.311 | 74.142 | 1.00 | 29.18 | 8 |
| ATOM | 17621 | N | PRO | G | 1122 | 70.152 | 101.777 | 72.459 | 1.00 | 24.86 | 7 |
| ATOM | 17622 | CD | PRO | G | 1122 | 70.535 | 102.239 | 71.118 | 1.00 | 16.24 | 6 |
| ATOM | 17623 | CA | PRO | G | 1122 | 69.516 | 102.866 | 73.196 | 1.00 | 26.04 | 6 |
| ATOM | 17624 | CB | PRO | G | 1122 | 69.845 | 104.066 | 72.343 | 1.00 | 17.24 | 6 |
| ATOM | 17625 | CG | PRO | G | 1122 | 69.737 | 103.504 | 70.984 | 1.00 | 15.41 | 6 |
| ATOM | 17626 | C | PRO | G | 1122 | 68.019 | 102.720 | 73.358 | 1.00 | 27.64 | 6 |
| ATOM | 17627 | O | PRO | G | 1122 | 67.317 | 102.390 | 72.407 | 1.00 | 27.62 | 8 |
| ATOM | 17628 | N | LEU | G | 1123 | 67.528 | 102.983 | 74.559 | 1.00 | 86.74 | 7 |
| ATOM | 17629 | CA | LEU | G | 1123 | 66.101 | 102.895 | 74.808 | 1.00 | 88.45 | 6 |
| ATOM | 17630 | CB | LEU | G | 1123 | 65.843 | 102.158 | 76.116 | 1.00 | 21.15 | 6 |
| ATOM | 17631 | CG | LEU | G | 1123 | 65.935 | 100.633 | 76.015 | 1.00 | 18.97 | 6 |
| ATOM | 17632 | CD1 | LEU | G | 1123 | 67.235 | 100.180 | 75.409 | 1.00 | 16.38 | 6 |
| ATOM | 17633 | CD2 | LEU | G | 1123 | 65.792 | 100.068 | 77.399 | 1.00 | 17.40 | 6 |
| ATOM | 17634 | C | LEU | G | 1123 | 65.540 | 104.308 | 74.850 | 1.00 | 91.01 | 6 |
| ATOM | 17635 | O | LEU | G | 1123 | 64.337 | 104.525 | 75.016 | 1.00 | 92.76 | 8 |
| ATOM | 17636 | N | PHE | G | 1124 | 66.443 | 105.270 | 74.699 | 1.00 | 48.00 | 7 |
| ATOM | 17637 | CA | PHE | G | 1124 | 66.096 | 106.682 | 74.677 | 1.00 | 47.33 | 6 |
| ATOM | 17638 | CB | PHE | G | 1124 | 66.771 | 107.425 | 75.832 | 1.00 | 29.41 | 6 |
| ATOM | 17639 | CG | PHE | G | 1124 | 66.153 | 107.188 | 77.182 | 1.00 | 26.55 | 6 |
| ATOM | 17640 | CD1 | PHE | G | 1124 | 65.100 | 107.969 | 77.623 | 1.00 | 25.74 | 6 |
| ATOM | 17641 | CD2 | PHE | G | 1124 | 66.660 | 106.210 | 78.036 | 1.00 | 25.58 | 6 |
| ATOM | 17642 | CE1 | PHE | G | 1124 | 64.560 | 107.783 | 78.902 | 1.00 | 25.32 | 6 |
| ATOM | 17643 | CE2 | PHE | G | 1124 | 66.126 | 106.015 | 79.314 | 1.00 | 24.75 | 6 |
| ATOM | 17644 | CZ | PHE | G | 1124 | 65.074 | 106.805 | 79.748 | 1.00 | 24.40 | 6 |
| ATOM | 17645 | C | PHE | G | 1124 | 66.706 | 107.170 | 73.370 | 1.00 | 49.32 | 6 |
| ATOM | 17646 | O | PHE | G | 1124 | 67.845 | 106.815 | 73.058 | 1.00 | 48.69 | 8 |
| ATOM | 17647 | N | GLN | G | 1125 | 65.954 | 107.964 | 72.610 | 1.00 | 50.63 | 7 |
| ATOM | 17648 | CA | GLN | G | 1125 | 66.434 | 108.526 | 71.343 | 1.00 | 54.26 | 6 |
| ATOM | 17649 | CB | GLN | G | 1125 | 65.771 | 107.834 | 70.149 | 1.00 | 111.66 | 6 |
| ATOM | 17650 | CG | GLN | G | 1125 | 66.695 | 106.889 | 69.378 | 1.00 | 115.56 | 6 |
| ATOM | 17651 | CD | GLN | G | 1125 | 67.815 | 107.617 | 68.649 | 1.00 | 116.51 | 6 |
| ATOM | 17652 | OE1 | GLN | G | 1125 | 67.568 | 108.404 | 67.732 | 1.00 | 117.20 | 8 |
| ATOM | 17653 | NE2 | GLN | G | 1125 | 69.053 | 107.355 | 69.054 | 1.00 | 116.80 | 7 |
| ATOM | 17654 | C | GLN | G | 1125 | 66.112 | 110.012 | 71.311 | 1.00 | 55.90 | 6 |
| ATOM | 17655 | O | GLN | G | 1125 | 65.425 | 110.518 | 72.203 | 1.00 | 55.43 | 8 |
| ATOM | 17656 | N | MET | G | 1126 | 66.590 | 110.723 | 70.292 | 1.00 | 64.27 | 7 |
| ATOM | 17657 | CA | MET | G | 1126 | 66.316 | 112.156 | 70.234 | 1.00 | 67.94 | 6 |
| ATOM | 17658 | CB | MET | G | 1126 | 67.529 | 112.938 | 70.719 | 1.00 | 71.09 | 6 |
| ATOM | 17659 | CG | MET | G | 1126 | 67.189 | 114.350 | 71.142 | 1.00 | 70.18 | 6 |
| ATOM | 17660 | SD | MET | G | 1126 | 68.650 | 115.351 | 71.215 | 1.00 | 70.84 | 16 |
| ATOM | 17661 | CE | MET | G | 1126 | 69.645 | 114.448 | 72.460 | 1.00 | 69.37 | 6 |
| ATOM | 17662 | C | MET | G | 1126 | 65.843 | 112.772 | 68.917 | 1.00 | 70.21 | 6 |
| ATOM | 17663 | O | MET | G | 1126 | 66.120 | 112.280 | 67.822 | 1.00 | 71.17 | 8 |
| ATOM | 17664 | N | ASP | G | 1127 | 65.136 | 113.886 | 69.069 | 1.00 | 83.97 | 7 |
| ATOM | 17665 | CA | ASP | G | 1127 | 64.571 | 114.660 | 67.973 | 1.00 | 85.60 | 6 |
| ATOM | 17666 | CB | ASP | G | 1127 | 63.141 | 115.068 | 68.372 | 1.00 | 90.36 | 6 |
| ATOM | 17667 | CG | ASP | G | 1127 | 62.640 | 116.303 | 67.648 | 1.00 | 90.91 | 6 |
| ATOM | 17668 | OD1 | ASP | G | 1127 | 63.040 | 117.425 | 68.032 | 1.00 | 90.86 | 8 |
| ATOM | 17669 | OD2 | ASP | G | 1127 | 61.836 | 116.149 | 66.701 | 1.00 | 90.61 | 8 |
| ATOM | 17670 | C | ASP | G | 1127 | 65.461 | 115.880 | 67.709 | 1.00 | 86.98 | 6 |
| ATOM | 17671 | O | ASP | G | 1127 | 65.397 | 116.873 | 68.437 | 1.00 | 87.91 | 8 |
| ATOM | 17672 | N | GLU | G | 1128 | 66.301 | 115.791 | 66.676 | 1.00 | 69.91 | 7 |
| ATOM | 17673 | CA | GLU | G | 1128 | 67.215 | 116.883 | 66.318 | 1.00 | 70.82 | 6 |
| ATOM | 17674 | CB | GLU | G | 1128 | 67.917 | 116.591 | 64.986 | 1.00 | 119.73 | 6 |
| ATOM | 17675 | CG | GLU | G | 1128 | 68.731 | 115.307 | 64.970 | 1.00 | 121.05 | 6 |
| ATOM | 17676 | CD | GLU | G | 1128 | 69.453 | 115.076 | 63.650 | 1.00 | 122.37 | 6 |
| ATOM | 17677 | OE1 | GLU | G | 1128 | 68.792 | 115.067 | 62.587 | 1.00 | 123.48 | 8 |
| ATOM | 17678 | OE2 | GLU | G | 1128 | 70.688 | 114.897 | 63.681 | 1.00 | 122.49 | 8 |
| ATOM | 17679 | C | GLU | G | 1128 | 66.499 | 118.227 | 66.216 | 1.00 | 71.11 | 6 |
| ATOM | 17680 | O | GLU | G | 1128 | 67.143 | 119.273 | 66.270 | 1.00 | 70.66 | 8 |
| ATOM | 17681 | N | VAL | G | 1129 | 65.173 | 118.182 | 66.062 | 1.00 | 27.26 | 7 |
| ATOM | 17682 | CA | VAL | G | 1129 | 64.337 | 119.374 | 65.953 | 1.00 | 28.36 | 6 |
| ATOM | 17683 | CB | VAL | G | 1129 | 62.840 | 119.013 | 65.839 | 1.00 | 195.38 | 6 |
| ATOM | 17684 | CG1 | VAL | G | 1129 | 61.997 | 120.277 | 65.767 | 1.00 | 196.97 | 6 |
| ATOM | 17685 | CG2 | VAL | G | 1129 | 62.607 | 118.150 | 64.614 | 1.00 | 196.60 | 6 |
| ATOM | 17686 | C | VAL | G | 1129 | 64.534 | 120.231 | 67.179 | 1.00 | 27.77 | 6 |
| ATOM | 17687 | O | VAL | G | 1129 | 65.180 | 121.267 | 67.103 | 1.00 | 27.79 | 8 |
| ATOM | 17688 | N | THR | G | 1130 | 63.980 | 119.803 | 68.310 | 1.00 | 79.94 | 7 |
| ATOM | 17689 | CA | THR | G | 1130 | 64.126 | 120.558 | 69.552 | 1.00 | 82.10 | 6 |
| ATOM | 17690 | CB | THR | G | 1130 | 62.772 | 121.030 | 70.086 | 1.00 | 111.33 | 6 |
| ATOM | 17691 | OG1 | THR | G | 1130 | 61.956 | 121.466 | 68.993 | 1.00 | 112.30 | 8 |
| ATOM | 17692 | CG2 | THR | G | 1130 | 62.969 | 122.195 | 71.055 | 1.00 | 112.94 | 6 |
| ATOM | 17693 | C | THR | G | 1130 | 64.795 | 119.692 | 70.609 | 1.00 | 83.35 | 6 |
| ATOM | 17694 | O | THR | G | 1130 | 64.146 | 119.186 | 71.520 | 1.00 | 84.33 | 8 |
| ATOM | 17695 | N | ARG | G | 1131 | 66.108 | 119.544 | 70.460 | 1.00 | 64.02 | 7 |
| ATOM | 17696 | CA | ARG | G | 1131 | 66.956 | 118.741 | 71.334 | 1.00 | 65.04 | 6 |
| ATOM | 17697 | CB | ARG | G | 1131 | 68.240 | 119.503 | 71.674 | 1.00 | 77.20 | 6 |
| ATOM | 17698 | CG | ARG | G | 1131 | 68.967 | 120.133 | 70.496 | 1.00 | 75.94 | 6 |
| ATOM | 17699 | CD | ARG | G | 1131 | 68.374 | 121.490 | 70.138 | 1.00 | 75.39 | 6 |
| ATOM | 17700 | NE | ARG | G | 1131 | 69.361 | 122.378 | 69.527 | 1.00 | 74.87 | 7 |
| ATOM | 17701 | CZ | ARG | G | 1131 | 70.494 | 122.753 | 70.119 | 1.00 | 75.39 | 6 |
| ATOM | 17702 | NH1 | ARG | G | 1131 | 70.789 | 122.321 | 71.333 | 1.00 | 75.70 | 7 |
| ATOM | 17703 | NH2 | ARG | G | 1131 | 71.342 | 123.563 | 69.505 | 1.00 | 76.69 | 7 |
| ATOM | 17704 | C | ARG | G | 1131 | 66.288 | 118.299 | 72.627 | 1.00 | 66.57 | 6 |
| ATOM | 17705 | O | ARG | G | 1131 | 66.400 | 118.972 | 73.650 | 1.00 | 67.44 | 8 |
| ATOM | 17706 | N | THR | G | 1132 | 65.606 | 117.160 | 72.590 | 1.00 | 110.70 | 7 |
| ATOM | 17707 | CA | THR | G | 1132 | 64.943 | 116.652 | 73.783 | 1.00 | 112.07 | 6 |
| ATOM | 17708 | CB | THR | G | 1132 | 63.589 | 117.358 | 74.014 | 1.00 | 142.97 | 6 |
| ATOM | 17709 | OG1 | THR | G | 1132 | 63.800 | 118.770 | 74.154 | 1.00 | 143.10 | 8 |
| ATOM | 17710 | CG2 | THR | G | 1132 | 62.922 | 116.827 | 75.277 | 1.00 | 143.32 | 6 |
| ATOM | 17711 | C | THR | G | 1132 | 64.703 | 115.151 | 73.702 | 1.00 | 112.98 | 6 |
| ATOM | 17712 | O | THR | G | 1132 | 63.730 | 114.707 | 73.093 | 1.00 | 114.26 | 8 |
| ATOM | 17713 | N | LEU | G | 1133 | 65.592 | 114.378 | 74.321 | 1.00 | 130.03 | 7 |
| ATOM | 17714 | CA | LEU | G | 1133 | 65.485 | 112.923 | 74.327 | 1.00 | 129.99 | 6 |
| ATOM | 17715 | CB | LEU | G | 1133 | 66.421 | 112.329 | 75.373 | 1.00 | 113.82 | 6 |
| ATOM | 17716 | CG | LEU | G | 1133 | 67.911 | 112.597 | 75.172 | 1.00 | 115.74 | 6 |
| ATOM | 17717 | CD1 | LEU | G | 1133 | 68.641 | 112.271 | 76.457 | 1.00 | 115.14 | 6 |
| ATOM | 17718 | CD2 | LEU | G | 1133 | 68.454 | 111.775 | 74.005 | 1.00 | 116.19 | 6 |
| ATOM | 17719 | C | LEU | G | 1133 | 64.063 | 112.492 | 74.634 | 1.00 | 129.63 | 6 |
| ATOM | 17720 | O | LEU | G | 1133 | 63.295 | 113.246 | 75.226 | 1.00 | 130.87 | 8 |
| ATOM | 17721 | N | ARG | G | 1134 | 63.720 | 111.273 | 74.237 | 1.00 | 58.57 | 7 |
| ATOM | 17722 | CA | ARG | G | 1134 | 62.381 | 110.747 | 74.463 | 1.00 | 57.49 | 6 |
| ATOM | 17723 | CB | ARG | G | 1134 | 61.524 | 110.974 | 73.213 | 1.00 | 165.44 | 6 |
| ATOM | 17724 | CG | ARG | G | 1134 | 60.141 | 110.320 | 73.225 | 1.00 | 167.83 | 6 |
| ATOM | 17725 | CD | ARG | G | 1134 | 59.116 | 111.095 | 74.049 | 1.00 | 169.50 | 6 |
| ATOM | 17726 | NE | ARG | G | 1134 | 57.753 | 110.639 | 73.767 | 1.00 | 171.23 | 7 |
| ATOM | 17727 | CZ | ARG | G | 1134 | 56.653 | 111.190 | 74.274 | 1.00 | 173.32 | 6 |
| ATOM | 17728 | NH1 | ARG | G | 1134 | 56.742 | 112.225 | 75.099 | 1.00 | 175.05 | 7 |
| ATOM | 17729 | NH2 | ARG | G | 1134 | 55.461 | 110.711 | 73.949 | 1.00 | 173.72 | 7 |
| ATOM | 17730 | C | ARG | G | 1134 | 62.455 | 109.258 | 74.781 | 1.00 | 56.04 | 6 |
| ATOM | 17731 | O | ARG | G | 1134 | 63.454 | 108.596 | 74.489 | 1.00 | 56.13 | 8 |
| ATOM | 17732 | N | LEU | G | 1135 | 61.406 | 108.735 | 75.403 | 1.00 | 72.40 | 7 |
| ATOM | 17733 | CA | LEU | G | 1135 | 61.364 | 107.316 | 75.719 | 1.00 | 71.34 | 6 |
| ATOM | 17734 | CB | LEU | G | 1135 | 60.195 | 107.015 | 76.662 | 1.00 | 151.60 | 6 |
| ATOM | 17735 | CG | LEU | G | 1135 | 59.601 | 108.189 | 77.455 | 1.00 | 153.45 | 6 |
| ATOM | 17736 | CD1 | LEU | G | 1135 | 58.546 | 107.668 | 78.419 | 1.00 | 153.65 | 6 |
| ATOM | 17737 | CD2 | LEU | G | 1135 | 60.688 | 108.921 | 78.224 | 1.00 | 154.75 | 6 |
| ATOM | 17738 | C | LEU | G | 1135 | 61.102 | 106.707 | 74.356 | 1.00 | 69.68 | 6 |
| ATOM | 17739 | O | LEU | G | 1135 | 60.004 | 106.861 | 73.829 | 1.00 | 69.74 | 8 |
| ATOM | 17740 | N | ARG | G | 1136 | 62.105 | 106.052 | 73.772 | 1.00 | 58.02 | 7 |
| ATOM | 17741 | CA | ARG | G | 1136 | 61.961 | 105.445 | 72.440 | 1.00 | 55.77 | 6 |
| ATOM | 17742 | CB | ARG | G | 1136 | 63.115 | 104.473 | 72.182 | 1.00 | 61.16 | 6 |
| ATOM | 17743 | CG | ARG | G | 1136 | 63.512 | 104.323 | 70.726 | 1.00 | 59.77 | 6 |
| ATOM | 17744 | CD | ARG | G | 1136 | 64.776 | 103.499 | 70.627 | 1.00 | 59.47 | 6 |
| ATOM | 17745 | NE | ARG | G | 1136 | 65.057 | 103.071 | 69.261 | 1.00 | 58.71 | 7 |
| ATOM | 17746 | CZ | ARG | G | 1136 | 65.798 | 102.007 | 68.954 | 1.00 | 59.04 | 6 |
| ATOM | 17747 | NH1 | ARG | G | 1136 | 66.330 | 101.265 | 69.917 | 1.00 | 58.01 | 7 |
| ATOM | 17748 | NH2 | ARG | G | 1136 | 66.002 | 101.672 | 67.685 | 1.00 | 59.42 | 7 |
| ATOM | 17749 | C | ARG | G | 1136 | 60.623 | 104.722 | 72.389 | 1.00 | 55.16 | 6 |
| ATOM | 17750 | O | ARG | G | 1136 | 60.118 | 104.300 | 73.428 | 1.00 | 55.68 | 8 |
| ATOM | 17751 | N | LYS | G | 1137 | 60.029 | 104.575 | 71.209 | 1.00 | 61.13 | 7 |
| ATOM | 17752 | CA | LYS | G | 1137 | 58.722 | 103.920 | 71.152 | 1.00 | 60.84 | 6 |
| ATOM | 17753 | CB | LYS | G | 1137 | 58.061 | 104.136 | 69.788 | 1.00 | 57.63 | 6 |
| ATOM | 17754 | CG | LYS | G | 1137 | 58.958 | 103.834 | 68.624 | 1.00 | 59.33 | 6 |
| ATOM | 17755 | CD | LYS | G | 1137 | 58.262 | 104.048 | 67.289 | 1.00 | 61.19 | 6 |
| ATOM | 17756 | CE | LYS | G | 1137 | 59.247 | 103.881 | 66.135 | 1.00 | 62.98 | 6 |
| ATOM | 17757 | NZ | LYS | G | 1137 | 58.628 | 104.071 | 64.800 | 1.00 | 64.64 | 7 |
| ATOM | 17758 | C | LYS | G | 1137 | 58.714 | 102.436 | 71.514 | 1.00 | 60.86 | 6 |
| ATOM | 17759 | O | LYS | G | 1137 | 59.581 | 101.656 | 71.102 | 1.00 | 60.08 | 8 |
| ATOM | 17760 | N | ARG | G | 1138 | 57.715 | 102.079 | 72.315 | 1.00 | 173.33 | 7 |
| ATOM | 17761 | CA | ARG | G | 1138 | 57.506 | 100.719 | 72.795 | 1.00 | 174.09 | 6 |
| ATOM | 17762 | CB | ARG | G | 1138 | 56.207 | 100.655 | 73.616 | 1.00 | 114.23 | 6 |
| ATOM | 17763 | CG | ARG | G | 1138 | 55.425 | 101.977 | 73.685 | 1.00 | 115.53 | 6 |
| ATOM | 17764 | CD | ARG | G | 1138 | 54.776 | 102.332 | 72.353 | 1.00 | 116.52 | 6 |
| ATOM | 17765 | NE | ARG | G | 1138 | 54.181 | 103.664 | 72.375 | 1.00 | 118.54 | 7 |
| ATOM | 17766 | CZ | ARG | G | 1138 | 53.394 | 104.148 | 71.419 | 1.00 | 119.91 | 6 |
| ATOM | 17767 | NH1 | ARG | G | 1138 | 53.097 | 103.413 | 70.357 | 1.00 | 119.84 | 7 |
| ATOM | 17768 | NH2 | ARG | G | 1138 | 52.901 | 105.372 | 71.523 | 1.00 | 120.68 | 7 |
| ATOM | 17769 | C | ARG | G | 1138 | 57.440 | 99.734 | 71.638 | 1.00 | 173.24 | 6 |
| ATOM | 17770 | O | ARG | G | 1138 | 56.417 | 99.089 | 71.414 | 1.00 | 175.43 | 8 |
| ATOM | 17771 | N | SER | G | 1139 | 58.544 | 99.621 | 70.909 | 1.00 | 90.68 | 7 |
| ATOM | 17772 | CA | SER | G | 1139 | 58.616 | 98.724 | 69.764 | 1.00 | 86.88 | 6 |
| ATOM | 17773 | CB | SER | G | 1139 | 57.782 | 99.269 | 68.593 | 1.00 | 36.28 | 6 |
| ATOM | 17774 | OG | SER | G | 1139 | 57.935 | 98.481 | 67.421 | 1.00 | 32.66 | 8 |
| ATOM | 17775 | C | SER | G | 1139 | 60.048 | 98.541 | 69.306 | 1.00 | 84.63 | 6 |
| ATOM | 17776 | O | SER | G | 1139 | 60.606 | 97.450 | 69.433 | 1.00 | 84.85 | 8 |
| ATOM | 17777 | N | ASP | G | 1140 | 60.638 | 99.614 | 68.777 | 1.00 | 58.25 | 7 |
| ATOM | 17778 | CA | ASP | G | 1140 | 62.006 | 99.562 | 68.270 | 1.00 | 53.75 | 6 |
| ATOM | 17779 | CB | ASP | G | 1140 | 62.511 | 100.959 | 67.959 | 1.00 | 66.26 | 6 |
| ATOM | 17780 | CG | ASP | G | 1140 | 61.431 | 101.847 | 67.418 | 1.00 | 66.48 | 6 |
| ATOM | 17781 | OD1 | ASP | G | 1140 | 60.700 | 101.406 | 66.503 | 1.00 | 66.87 | 8 |
| ATOM | 17782 | OD2 | ASP | G | 1140 | 61.322 | 102.988 | 67.906 | 1.00 | 67.08 | 8 |
| ATOM | 17783 | C | ASP | G | 1140 | 62.910 | 98.918 | 69.290 | 1.00 | 50.32 | 6 |
| ATOM | 17784 | O | ASP | G | 1140 | 63.761 | 98.102 | 68.948 | 1.00 | 49.17 | 8 |
| ATOM | 17785 | N | ILE | G | 1141 | 62.722 | 99.299 | 70.547 | 1.00 | 40.29 | 7 |
| ATOM | 17786 | CA | ILE | G | 1141 | 63.510 | 98.739 | 71.626 | 1.00 | 37.07 | 6 |
| ATOM | 17787 | CB | ILE | G | 1141 | 62.913 | 99.100 | 72.981 | 1.00 | 21.59 | 6 |
| ATOM | 17788 | CG2 | ILE | G | 1141 | 63.772 | 98.520 | 74.096 | 1.00 | 20.43 | 6 |
| ATOM | 17789 | CG1 | ILE | G | 1141 | 62.775 | 100.608 | 73.096 | 1.00 | 20.52 | 6 |
| ATOM | 17790 | CD1 | ILE | G | 1141 | 62.006 | 101.005 | 74.286 | 1.00 | 23.36 | 6 |
| ATOM | 17791 | C | ILE | G | 1141 | 63.430 | 97.230 | 71.469 | 1.00 | 36.64 | 6 |
| ATOM | 17792 | O | ILE | G | 1141 | 64.448 | 96.548 | 71.339 | 1.00 | 36.06 | 8 |
| ATOM | 17793 | N | GLU | G | 1142 | 62.191 | 96.736 | 71.478 | 1.00 | 58.56 | 7 |
| ATOM | 17794 | CA | GLU | G | 1142 | 61.870 | 95.315 | 71.337 | 1.00 | 57.76 | 6 |
| ATOM | 17795 | CB | GLU | G | 1142 | 60.365 | 95.122 | 71.424 | 1.00 | 66.98 | 6 |
| ATOM | 17796 | CG | GLU | G | 1142 | 59.934 | 93.705 | 71.251 | 1.00 | 68.24 | 6 |
| ATOM | 17797 | CD | GLU | G | 1142 | 58.440 | 93.582 | 71.235 | 1.00 | 69.98 | 6 |
| ATOM | 17798 | OE1 | GLU | G | 1142 | 57.799 | 94.063 | 72.193 | 1.00 | 71.80 | 8 |
| ATOM | 17799 | OE2 | GLU | G | 1142 | 57.902 | 93.004 | 70.271 | 1.00 | 70.75 | 8 |
| ATOM | 17800 | C | GLU | G | 1142 | 62.347 | 94.802 | 70.003 | 1.00 | 55.54 | 6 |
| ATOM | 17801 | O | GLU | G | 1142 | 62.745 | 93.649 | 69.864 | 1.00 | 54.54 | 8 |
| ATOM | 17802 | N | SER | G | 1143 | 62.277 | 95.686 | 69.022 | 1.00 | 45.57 | 7 |
| ATOM | 17803 | CA | SER | G | 1143 | 62.705 | 95.385 | 67.679 | 1.00 | 42.50 | 6 |
| ATOM | 17804 | CB | SER | G | 1143 | 62.168 | 96.447 | 66.715 | 1.00 | 23.64 | 6 |
| ATOM | 17805 | OG | SER | G | 1143 | 61.788 | 95.882 | 65.473 | 1.00 | 20.91 | 8 |
| ATOM | 17806 | C | SER | G | 1143 | 64.226 | 95.411 | 67.712 | 1.00 | 40.89 | 6 |
| ATOM | 17807 | O | SER | G | 1143 | 64.878 | 94.874 | 66.828 | 1.00 | 41.57 | 8 |
| ATOM | 17808 | N | GLY | G | 1144 | 64.783 | 96.023 | 68.753 | 1.00 | 32.49 | 7 |
| ATOM | 17809 | CA | GLY | G | 1144 | 66.229 | 96.102 | 68.887 | 1.00 | 32.11 | 6 |
| ATOM | 17810 | C | GLY | G | 1144 | 66.815 | 95.201 | 69.966 | 1.00 | 31.50 | 6 |
| ATOM | 17811 | O | GLY | G | 1144 | 68.032 | 95.009 | 70.052 | 1.00 | 31.35 | 8 |
| ATOM | 17812 | N | LEU | G | 1145 | 65.944 | 94.624 | 70.782 | 1.00 | 57.42 | 7 |
| ATOM | 17813 | CA | LEU | G | 1145 | 66.393 | 93.763 | 71.853 | 1.00 | 55.12 | 6 |
| ATOM | 17814 | CB | LEU | G | 1145 | 65.964 | 94.346 | 73.193 | 1.00 | 16.96 | 6 |
| ATOM | 17815 | CG | LEU | G | 1145 | 66.481 | 95.743 | 73.548 | 1.00 | 14.26 | 6 |
| ATOM | 17816 | CD1 | LEU | G | 1145 | 65.982 | 96.114 | 74.936 | 1.00 | 13.87 | 6 |
| ATOM | 17817 | CD2 | LEU | G | 1145 | 68.001 | 95.775 | 73.492 | 1.00 | 13.87 | 6 |
| ATOM | 17818 | C | LEU | G | 1145 | 65.906 | 92.328 | 71.752 | 1.00 | 55.41 | 6 |
| ATOM | 17819 | O | LEU | G | 1145 | 66.695 | 91.396 | 71.897 | 1.00 | 57.87 | 8 |
| ATOM | 17820 | N | TYR | G | 1146 | 64.614 | 92.137 | 71.504 | 1.00 | 29.27 | 7 |
| ATOM | 17821 | CA | TYR | G | 1146 | 64.066 | 90.777 | 71.418 | 1.00 | 27.78 | 6 |
| ATOM | 17822 | CB | TYR | G | 1146 | 62.729 | 90.781 | 70.682 | 1.00 | 21.95 | 6 |
| ATOM | 17823 | CG | TYR | G | 1146 | 62.075 | 89.423 | 70.597 | 1.00 | 20.28 | 6 |
| ATOM | 17824 | CD1 | TYR | G | 1146 | 60.697 | 89.308 | 70.495 | 1.00 | 20.32 | 6 |
| ATOM | 17825 | CE1 | TYR | G | 1146 | 60.088 | 88.063 | 70.389 | 1.00 | 19.89 | 6 |
| ATOM | 17826 | CD2 | TYR | G | 1146 | 62.829 | 88.254 | 70.594 | 1.00 | 19.89 | 6 |
| ATOM | 17827 | CE2 | TYR | G | 1146 | 62.237 | 87.006 | 70.489 | 1.00 | 19.67 | 6 |
| ATOM | 17828 | CZ | TYR | G | 1146 | 60.864 | 86.911 | 70.382 | 1.00 | 20.14 | 6 |
| ATOM | 17829 | OH | TYR | G | 1146 | 60.268 | 85.669 | 70.230 | 1.00 | 19.11 | 8 |
| ATOM | 17830 | C | TYR | G | 1146 | 64.973 | 89.752 | 70.755 | 1.00 | 28.02 | 6 |
| ATOM | 17831 | O | TYR | G | 1146 | 65.232 | 89.832 | 69.570 | 1.00 | 28.65 | 8 |
| ATOM | 17832 | N | GLY | G | 1147 | 65.431 | 88.767 | 71.510 | 1.00 | 44.62 | 7 |
| ATOM | 17833 | CA | GLY | G | 1147 | 66.268 | 87.755 | 70.904 | 1.00 | 48.07 | 6 |
| ATOM | 17834 | C | GLY | G | 1147 | 67.758 | 87.989 | 71.027 | 1.00 | 50.10 | 6 |
| ATOM | 17835 | O | GLY | G | 1147 | 68.563 | 87.165 | 70.576 | 1.00 | 52.13 | 8 |
| ATOM | 17836 | N | ARG | G | 1148 | 68.140 | 89.104 | 71.634 | 1.00 | 38.18 | 7 |
| ATOM | 17837 | CA | ARG | G | 1148 | 69.550 | 89.407 | 71.819 | 1.00 | 39.55 | 6 |
| ATOM | 17838 | CB | ARG | G | 1148 | 69.710 | 90.876 | 72.159 | 1.00 | 42.03 | 6 |
| ATOM | 17839 | CG | ARG | G | 1148 | 69.228 | 91.849 | 71.113 | 1.00 | 42.98 | 6 |
| ATOM | 17840 | CD | ARG | G | 1148 | 70.093 | 91.802 | 69.879 | 1.00 | 44.28 | 6 |
| ATOM | 17841 | NE | ARG | G | 1148 | 70.036 | 93.067 | 69.161 | 1.00 | 45.14 | 7 |
| ATOM | 17842 | CZ | ARG | G | 1148 | 70.794 | 93.351 | 68.112 | 1.00 | 45.75 | 6 |
| ATOM | 17843 | NH1 | ARG | G | 1148 | 71.668 | 92.454 | 67.659 | 1.00 | 45.83 | 7 |
| ATOM | 17844 | NH2 | ARG | G | 1148 | 70.681 | 94.534 | 67.524 | 1.00 | 45.78 | 7 |
| ATOM | 17845 | C | ARG | G | 1148 | 70.149 | 88.571 | 72.952 | 1.00 | 40.39 | 6 |
| ATOM | 17846 | O | ARG | G | 1148 | 69.444 | 87.892 | 73.688 | 1.00 | 40.17 | 8 |
| ATOM | 17847 | N | VAL | G | 1149 | 71.461 | 88.645 | 73.093 | 1.00 | 29.90 | 7 |
| ATOM | 17848 | CA | VAL | G | 1149 | 72.181 | 87.924 | 74.137 | 1.00 | 31.96 | 6 |
| ATOM | 17849 | CB | VAL | G | 1149 | 73.099 | 86.832 | 73.479 | 1.00 | 29.71 | 6 |
| ATOM | 17850 | CG1 | VAL | G | 1149 | 74.543 | 86.929 | 73.986 | 1.00 | 28.46 | 6 |
| ATOM | 17851 | CG2 | VAL | G | 1149 | 72.508 | 85.443 | 73.727 | 1.00 | 28.35 | 6 |
| ATOM | 17852 | C | VAL | G | 1149 | 72.989 | 88.984 | 74.911 | 1.00 | 34.86 | 6 |
| ATOM | 17853 | O | VAL | G | 1149 | 73.950 | 89.553 | 74.386 | 1.00 | 35.46 | 8 |
| ATOM | 17854 | N | LEU | G | 1150 | 72.587 | 89.271 | 76.147 | 1.00 | 50.78 | 7 |
| ATOM | 17855 | CA | LEU | G | 1150 | 73.272 | 90.291 | 76.934 | 1.00 | 54.05 | 6 |
| ATOM | 17856 | CB | LEU | G | 1150 | 72.693 | 90.371 | 78.347 | 1.00 | 36.77 | 6 |
| ATOM | 17857 | CG | LEU | G | 1150 | 71.419 | 91.206 | 78.526 | 1.00 | 36.53 | 6 |
| ATOM | 17858 | CD1 | LEU | G | 1150 | 71.212 | 91.485 | 80.010 | 1.00 | 36.78 | 6 |
| ATOM | 17859 | CD2 | LEU | G | 1150 | 71.535 | 92.521 | 77.776 | 1.00 | 35.95 | 6 |
| ATOM | 17860 | C | LEU | G | 1150 | 74.776 | 90.099 | 77.006 | 1.00 | 56.56 | 6 |
| ATOM | 17861 | O | LEU | G | 1150 | 75.265 | 88.977 | 77.055 | 1.00 | 57.24 | 8 |
| ATOM | 17862 | N | ALA | G | 1151 | 75.503 | 91.211 | 77.013 | 1.00 | 68.82 | 7 |
| ATOM | 17863 | CA | ALA | G | 1151 | 76.957 | 91.176 | 77.053 | 1.00 | 71.07 | 6 |
| ATOM | 17864 | CB | ALA | G | 1151 | 77.503 | 91.823 | 75.804 | 1.00 | 36.93 | 6 |
| ATOM | 17865 | C | ALA | G | 1151 | 77.536 | 91.843 | 78.309 | 1.00 | 72.81 | 6 |
| ATOM | 17866 | O | ALA | G | 1151 | 78.667 | 92.361 | 78.319 | 1.00 | 74.07 | 8 |
| ATOM | 17867 | N | ARG | G | 1152 | 76.737 | 91.820 | 79.369 | 1.00 | 80.03 | 7 |
| ATOM | 17868 | CA | ARG | G | 1152 | 77.121 | 92.368 | 80.662 | 1.00 | 81.05 | 6 |
| ATOM | 17869 | CB | ARG | G | 1152 | 77.810 | 93.733 | 80.503 | 1.00 | 120.97 | 6 |
| ATOM | 17870 | CG | ARG | G | 1152 | 79.046 | 93.904 | 81.401 | 1.00 | 124.97 | 6 |
| ATOM | 17871 | CD | ARG | G | 1152 | 79.642 | 95.313 | 81.317 | 1.00 | 127.44 | 6 |
| ATOM | 17872 | NE | ARG | G | 1152 | 80.720 | 95.525 | 82.285 | 1.00 | 127.30 | 7 |
| ATOM | 17873 | CZ | ARG | G | 1152 | 81.319 | 96.694 | 82.505 | 1.00 | 127.42 | 6 |
| ATOM | 17874 | NH1 | ARG | G | 1152 | 80.956 | 97.779 | 81.831 | 1.00 | 127.13 | 7 |
| ATOM | 17875 | NH2 | ARG | G | 1152 | 82.285 | 96.784 | 83.407 | 1.00 | 127.90 | 7 |
| ATOM | 17876 | C | ARG | G | 1152 | 75.856 | 92.489 | 81.500 | 1.00 | 79.74 | 6 |
| ATOM | 17877 | O | ARG | G | 1152 | 74.945 | 93.238 | 81.159 | 1.00 | 79.94 | 8 |
| ATOM | 17878 | N | GLU | G | 1153 | 75.811 | 91.720 | 82.582 | 1.00 | 45.10 | 7 |
| ATOM | 17879 | CA | GLU | G | 1153 | 74.679 | 91.689 | 83.498 | 1.00 | 44.26 | 6 |
| ATOM | 17880 | CB | GLU | G | 1153 | 75.180 | 91.307 | 84.879 | 1.00 | 87.58 | 6 |
| ATOM | 17881 | CG | GLU | G | 1153 | 76.115 | 92.330 | 85.467 | 1.00 | 92.13 | 6 |
| ATOM | 17882 | CD | GLU | G | 1153 | 76.857 | 91.795 | 86.663 | 1.00 | 95.17 | 6 |
| ATOM | 17883 | OE1 | GLU | G | 1153 | 76.216 | 91.116 | 87.494 | 1.00 | 96.43 | 8 |
| ATOM | 17884 | OE2 | GLU | G | 1153 | 78.076 | 92.056 | 86.775 | 1.00 | 97.56 | 8 |
| ATOM | 17885 | C | GLU | G | 1153 | 73.949 | 93.024 | 83.571 | 1.00 | 42.37 | 6 |
| ATOM | 17886 | O | GLU | G | 1153 | 74.583 | 94.066 | 83.575 | 1.00 | 42.38 | 8 |
| ATOM | 17887 | N | VAL | G | 1154 | 72.620 | 92.996 | 83.627 | 1.00 | 18.51 | 7 |
| ATOM | 17888 | CA | VAL | G | 1154 | 71.837 | 94.226 | 83.699 | 1.00 | 15.82 | 6 |
| ATOM | 17889 | CB | VAL | G | 1154 | 70.963 | 94.433 | 82.422 | 1.00 | 13.87 | 6 |
| ATOM | 17890 | CG1 | VAL | G | 1154 | 69.832 | 95.439 | 82.693 | 1.00 | 13.87 | 6 |
| ATOM | 17891 | CG2 | VAL | G | 1154 | 71.826 | 94.924 | 81.267 | 1.00 | 13.87 | 6 |
| ATOM | 17892 | C | VAL | G | 1154 | 70.921 | 94.187 | 84.903 | 1.00 | 16.07 | 6 |
| ATOM | 17893 | O | VAL | G | 1154 | 69.783 | 93.780 | 84.790 | 1.00 | 15.97 | 8 |
| ATOM | 17894 | N | GLU | G | 1155 | 71.418 | 94.601 | 86.061 | 1.00 | 38.50 | 7 |
| ATOM | 17895 | CA | GLU | G | 1155 | 70.603 | 94.626 | 87.271 | 1.00 | 39.28 | 6 |
| ATOM | 17896 | CB | GLU | G | 1155 | 71.479 | 94.603 | 88.525 | 1.00 | 129.76 | 6 |
| ATOM | 17897 | CG | GLU | G | 1155 | 72.887 | 94.068 | 88.327 | 1.00 | 134.02 | 6 |
| ATOM | 17898 | CD | GLU | G | 1155 | 73.847 | 95.129 | 87.828 | 1.00 | 136.34 | 6 |
| ATOM | 17899 | OE1 | GLU | G | 1155 | 73.929 | 96.202 | 88.465 | 1.00 | 136.70 | 8 |
| ATOM | 17900 | OE2 | GLU | G | 1155 | 74.529 | 94.891 | 86.807 | 1.00 | 137.03 | 8 |
| ATOM | 17901 | C | GLU | G | 1155 | 69.828 | 95.931 | 87.242 | 1.00 | 38.44 | 6 |
| ATOM | 17902 | O | GLU | G | 1155 | 70.275 | 96.902 | 86.643 | 1.00 | 37.81 | 8 |
| ATOM | 17903 | N | ALA | G | 1156 | 68.673 | 95.950 | 87.891 | 1.00 | 34.56 | 7 |
| ATOM | 17904 | CA | ALA | G | 1156 | 67.836 | 97.139 | 87.941 | 1.00 | 35.23 | 6 |
| ATOM | 17905 | CB | ALA | G | 1156 | 67.807 | 97.830 | 86.600 | 1.00 | 54.56 | 6 |
| ATOM | 17906 | C | ALA | G | 1156 | 66.431 | 96.749 | 88.324 | 1.00 | 36.34 | 6 |
| ATOM | 17907 | O | ALA | G | 1156 | 66.097 | 95.566 | 88.354 | 1.00 | 36.10 | 8 |
| ATOM | 17908 | N | LEU | G | 1157 | 65.615 | 97.760 | 88.601 | 1.00 | 41.55 | 7 |
| ATOM | 17909 | CA | LEU | G | 1157 | 64.219 | 97.585 | 88.988 | 1.00 | 43.21 | 6 |
| ATOM | 17910 | CB | LEU | G | 1157 | 63.319 | 97.821 | 87.772 | 1.00 | 66.43 | 6 |
| ATOM | 17911 | CG | LEU | G | 1157 | 61.938 | 98.419 | 88.049 | 1.00 | 67.76 | 6 |
| ATOM | 17912 | CD1 | LEU | G | 1157 | 61.384 | 99.005 | 86.764 | 1.00 | 68.31 | 6 |
| ATOM | 17913 | CD2 | LEU | G | 1157 | 60.996 | 97.367 | 88.631 | 1.00 | 68.02 | 6 |
| ATOM | 17914 | C | LEU | G | 1157 | 63.961 | 96.206 | 89.586 | 1.00 | 44.00 | 6 |
| ATOM | 17915 | O | LEU | G | 1157 | 63.078 | 95.477 | 89.147 | 1.00 | 43.44 | 8 |
| ATOM | 17916 | N | GLY | G | 1158 | 64.765 | 95.847 | 90.581 | 1.00 | 57.48 | 7 |
| ATOM | 17917 | CA | GLY | G | 1158 | 64.598 | 94.569 | 91.243 | 1.00 | 59.72 | 6 |
| ATOM | 17918 | C | GLY | G | 1158 | 65.497 | 93.443 | 90.778 | 1.00 | 61.27 | 6 |
| ATOM | 17919 | O | GLY | G | 1158 | 66.308 | 92.925 | 91.558 | 1.00 | 61.87 | 8 |
| ATOM | 17920 | N | ARG | G | 1159 | 65.374 | 93.072 | 89.506 | 1.00 | 48.58 | 7 |
| ATOM | 17921 | CA | ARG | G | 1159 | 66.146 | 91.954 | 88.966 | 1.00 | 50.73 | 6 |
| ATOM | 17922 | CB | ARG | G | 1159 | 65.312 | 91.182 | 87.936 | 1.00 | 101.21 | 6 |
| ATOM | 17923 | CG | ARG | G | 1159 | 63.888 | 90.848 | 88.399 | 1.00 | 103.69 | 6 |
| ATOM | 17924 | CD | ARG | G | 1159 | 63.844 | 90.283 | 89.825 | 1.00 | 103.94 | 6 |
| ATOM | 17925 | NE | ARG | G | 1159 | 62.476 | 90.060 | 90.294 | 1.00 | 103.85 | 7 |
| ATOM | 17926 | CZ | ARG | G | 1159 | 62.158 | 89.753 | 91.547 | 1.00 | 104.43 | 6 |
| ATOM | 17927 | NH1 | ARG | G | 1159 | 63.105 | 89.636 | 92.466 | 1.00 | 105.91 | 7 |
| ATOM | 17928 | NH2 | ARG | G | 1159 | 60.894 | 89.550 | 91.881 | 1.00 | 103.85 | 7 |
| ATOM | 17929 | C | ARG | G | 1159 | 67.495 | 92.277 | 88.360 | 1.00 | 51.23 | 6 |
| ATOM | 17930 | O | ARG | G | 1159 | 67.775 | 93.415 | 87.990 | 1.00 | 52.62 | 8 |
| ATOM | 17931 | N | ARG | G | 1160 | 68.332 | 91.246 | 88.294 | 1.00 | 37.06 | 7 |
| ATOM | 17932 | CA | ARG | G | 1160 | 69.666 | 91.333 | 87.718 | 1.00 | 38.02 | 6 |
| ATOM | 17933 | CB | ARG | G | 1160 | 70.740 | 90.960 | 88.743 | 1.00 | 149.97 | 6 |
| ATOM | 17934 | CG | ARG | G | 1160 | 70.837 | 91.906 | 89.921 | 1.00 | 154.14 | 6 |
| ATOM | 17935 | CD | ARG | G | 1160 | 72.215 | 91.837 | 90.562 | 1.00 | 157.15 | 6 |
| ATOM | 17936 | NE | ARG | G | 1160 | 72.470 | 92.990 | 91.420 | 1.00 | 160.00 | 7 |
| ATOM | 17937 | CZ | ARG | G | 1160 | 73.652 | 93.274 | 91.959 | 1.00 | 162.29 | 6 |
| ATOM | 17938 | NH1 | ARG | G | 1160 | 74.694 | 92.487 | 91.728 | 1.00 | 163.57 | 7 |
| ATOM | 17939 | NH2 | ARG | G | 1160 | 73.796 | 94.345 | 92.727 | 1.00 | 162.30 | 7 |
| ATOM | 17940 | C | ARG | G | 1160 | 69.646 | 90.295 | 86.613 | 1.00 | 37.25 | 6 |
| ATOM | 17941 | O | ARG | G | 1160 | 69.366 | 89.125 | 86.877 | 1.00 | 36.75 | 8 |
| ATOM | 17942 | N | LEU | G | 1161 | 69.931 | 90.720 | 85.385 | 1.00 | 54.98 | 7 |
| ATOM | 17943 | CA | LEU | G | 1161 | 69.919 | 89.817 | 84.235 | 1.00 | 55.36 | 6 |
| ATOM | 17944 | CB | LEU | G | 1161 | 69.287 | 90.521 | 83.032 | 1.00 | 39.02 | 6 |
| ATOM | 17945 | CG | LEU | G | 1161 | 68.000 | 91.332 | 83.211 | 1.00 | 38.07 | 6 |
| ATOM | 17946 | CD1 | LEU | G | 1161 | 67.606 | 91.876 | 81.862 | 1.00 | 37.72 | 6 |
| ATOM | 17947 | CD2 | LEU | G | 1161 | 66.877 | 90.491 | 83.788 | 1.00 | 39.48 | 6 |
| ATOM | 17948 | C | LEU | G | 1161 | 71.329 | 89.362 | 83.868 | 1.00 | 55.94 | 6 |
| ATOM | 17949 | O | LEU | G | 1161 | 72.061 | 90.092 | 83.202 | 1.00 | 55.42 | 8 |
| ATOM | 17950 | N | GLU | G | 1162 | 71.697 | 88.152 | 84.290 | 1.00 | 55.76 | 7 |
| ATOM | 17951 | CA | GLU | G | 1162 | 73.029 | 87.615 | 84.023 | 1.00 | 57.35 | 6 |
| ATOM | 17952 | CB | GLU | G | 1162 | 73.070 | 86.108 | 84.302 | 1.00 | 136.81 | 6 |
| ATOM | 17953 | CG | GLU | G | 1162 | 71.720 | 85.414 | 84.278 | 1.00 | 139.78 | 6 |
| ATOM | 17954 | CD | GLU | G | 1162 | 71.846 | 83.900 | 84.317 | 1.00 | 142.06 | 6 |
| ATOM | 17955 | OE1 | GLU | G | 1162 | 71.912 | 83.280 | 83.233 | 1.00 | 143.37 | 8 |
| ATOM | 17956 | OE2 | GLU | G | 1162 | 71.893 | 83.332 | 85.431 | 1.00 | 144.06 | 8 |
| ATOM | 17957 | C | GLU | G | 1162 | 73.518 | 87.893 | 82.605 | 1.00 | 57.20 | 6 |
| ATOM | 17958 | O | GLU | G | 1162 | 72.713 | 88.089 | 81.688 | 1.00 | 57.41 | 8 |
| ATOM | 17959 | N | GLU | G | 1163 | 74.843 | 87.911 | 82.439 | 1.00 | 38.52 | 7 |
| ATOM | 17960 | CA | GLU | G | 1163 | 75.463 | 88.180 | 81.146 | 1.00 | 37.84 | 6 |
| ATOM | 17961 | CB | GLU | G | 1163 | 76.963 | 88.448 | 81.298 | 1.00 | 95.66 | 6 |
| ATOM | 17962 | CG | GLU | G | 1163 | 77.576 | 89.123 | 80.078 | 1.00 | 98.93 | 6 |
| ATOM | 17963 | CD | GLU | G | 1163 | 78.718 | 88.343 | 79.454 | 1.00 | 102.02 | 6 |
| ATOM | 17964 | OE1 | GLU | G | 1163 | 79.711 | 88.066 | 80.160 | 1.00 | 103.81 | 8 |
| ATOM | 17965 | OE2 | GLU | G | 1163 | 78.625 | 88.016 | 78.249 | 1.00 | 104.08 | 8 |
| ATOM | 17966 | C | GLU | G | 1163 | 75.270 | 87.005 | 80.221 | 1.00 | 36.30 | 6 |
| ATOM | 17967 | O | GLU | G | 1163 | 75.274 | 85.860 | 80.664 | 1.00 | 35.69 | 8 |
| ATOM | 17968 | N | GLY | G | 1164 | 75.096 | 87.292 | 78.935 | 1.00 | 72.10 | 7 |
| ATOM | 17969 | CA | GLY | G | 1164 | 74.926 | 86.237 | 77.954 | 1.00 | 71.36 | 6 |
| ATOM | 17970 | C | GLY | G | 1164 | 73.515 | 85.705 | 77.820 | 1.00 | 70.58 | 6 |
| ATOM | 17971 | O | GLY | G | 1164 | 73.186 | 85.055 | 76.837 | 1.00 | 71.82 | 8 |
| ATOM | 17972 | N | ARG | G | 1165 | 72.672 | 85.960 | 78.804 | 1.00 | 65.61 | 7 |
| ATOM | 17973 | CA | ARG | G | 1165 | 71.308 | 85.477 | 78.728 | 1.00 | 64.60 | 6 |
| ATOM | 17974 | CB | ARG | G | 1165 | 70.574 | 85.866 | 80.001 | 1.00 | 143.03 | 6 |
| ATOM | 17975 | CG | ARG | G | 1165 | 69.082 | 85.674 | 79.940 | 1.00 | 145.42 | 6 |
| ATOM | 17976 | CD | ARG | G | 1165 | 68.427 | 86.446 | 81.050 | 1.00 | 146.39 | 6 |
| ATOM | 17977 | NE | ARG | G | 1165 | 66.979 | 86.364 | 80.984 | 1.00 | 149.32 | 7 |
| ATOM | 17978 | CZ | ARG | G | 1165 | 66.175 | 87.024 | 81.804 | 1.00 | 151.35 | 6 |
| ATOM | 17979 | NH1 | ARG | G | 1165 | 66.691 | 87.809 | 82.738 | 1.00 | 152.47 | 7 |
| ATOM | 17980 | NH2 | ARG | G | 1165 | 64.862 | 86.894 | 81.700 | 1.00 | 152.96 | 7 |
| ATOM | 17981 | C | ARG | G | 1165 | 70.615 | 86.104 | 77.517 | 1.00 | 62.91 | 6 |
| ATOM | 17982 | O | ARG | G | 1165 | 71.052 | 87.151 | 77.032 | 1.00 | 64.15 | 8 |
| ATOM | 17983 | N | TYR | G | 1166 | 69.553 | 85.464 | 77.019 | 1.00 | 55.02 | 7 |
| ATOM | 17984 | CA | TYR | G | 1166 | 68.783 | 85.999 | 75.887 | 1.00 | 51.39 | 6 |
| ATOM | 17985 | CB | TYR | G | 1166 | 68.379 | 84.912 | 74.902 | 1.00 | 32.01 | 6 |
| ATOM | 17986 | CG | TYR | G | 1166 | 69.454 | 84.068 | 74.241 | 1.00 | 30.78 | 6 |
| ATOM | 17987 | CD1 | TYR | G | 1166 | 70.109 | 83.063 | 74.938 | 1.00 | 30.12 | 6 |
| ATOM | 17988 | CE1 | TYR | G | 1166 | 70.874 | 82.116 | 74.265 | 1.00 | 28.63 | 6 |
| ATOM | 17989 | CD2 | TYR | G | 1166 | 69.628 | 84.113 | 72.855 | 1.00 | 29.93 | 6 |
| ATOM | 17990 | CE2 | TYR | G | 1166 | 70.384 | 83.177 | 72.190 | 1.00 | 28.19 | 6 |
| ATOM | 17991 | CZ | TYR | G | 1166 | 70.991 | 82.178 | 72.893 | 1.00 | 27.43 | 6 |
| ATOM | 17992 | OH | TYR | G | 1166 | 71.638 | 81.194 | 72.200 | 1.00 | 26.37 | 8 |
| ATOM | 17993 | C | TYR | G | 1166 | 67.468 | 86.587 | 76.412 | 1.00 | 49.63 | 6 |
| ATOM | 17994 | O | TYR | G | 1166 | 66.682 | 85.871 | 77.038 | 1.00 | 48.97 | 8 |
| ATOM | 17995 | N | LEU | G | 1167 | 67.201 | 87.866 | 76.152 | 1.00 | 38.55 | 7 |
| ATOM | 17996 | CA | LEU | G | 1167 | 65.948 | 88.441 | 76.641 | 1.00 | 36.68 | 6 |
| ATOM | 17997 | CB | LEU | G | 1167 | 66.052 | 89.966 | 76.909 | 1.00 | 18.86 | 6 |
| ATOM | 17998 | CG | LEU | G | 1167 | 66.973 | 91.014 | 76.279 | 1.00 | 17.61 | 6 |
| ATOM | 17999 | CD1 | LEU | G | 1167 | 68.296 | 91.045 | 77.011 | 1.00 | 16.78 | 6 |
| ATOM | 18000 | CD2 | LEU | G | 1167 | 67.137 | 90.737 | 74.818 | 1.00 | 18.49 | 6 |
| ATOM | 18001 | C | LEU | G | 1167 | 64.742 | 88.164 | 75.753 | 1.00 | 35.58 | 6 |
| ATOM | 18002 | O | LEU | G | 1167 | 64.769 | 88.398 | 74.547 | 1.00 | 35.26 | 8 |
| ATOM | 18003 | N | SER | G | 1168 | 63.684 | 87.654 | 76.375 | 1.00 | 33.21 | 7 |
| ATOM | 18004 | CA | SER | G | 1168 | 62.442 | 87.343 | 75.684 | 1.00 | 34.10 | 6 |
| ATOM | 18005 | CB | SER | G | 1168 | 61.685 | 86.244 | 76.438 | 1.00 | 176.53 | 6 |
| ATOM | 18006 | OG | SER | G | 1168 | 62.478 | 85.080 | 76.596 | 1.00 | 181.29 | 8 |
| ATOM | 18007 | C | SER | G | 1168 | 61.568 | 88.597 | 75.594 | 1.00 | 33.27 | 6 |
| ATOM | 18008 | O | SER | G | 1168 | 61.861 | 89.614 | 76.220 | 1.00 | 32.22 | 8 |
| ATOM | 18009 | N | LEU | G | 1169 | 60.499 | 88.522 | 74.806 | 1.00 | 24.19 | 7 |
| ATOM | 18010 | CA | LEU | G | 1169 | 59.592 | 89.640 | 74.661 | 1.00 | 23.70 | 6 |
| ATOM | 18011 | CB | LEU | G | 1169 | 58.363 | 89.221 | 73.862 | 1.00 | 36.88 | 6 |
| ATOM | 18012 | CG | LEU | G | 1169 | 57.267 | 90.289 | 73.903 | 1.00 | 36.95 | 6 |
| ATOM | 18013 | CD1 | LEU | G | 1169 | 57.729 | 91.451 | 73.092 | 1.00 | 36.83 | 6 |
| ATOM | 18014 | CD2 | LEU | G | 1169 | 55.948 | 89.772 | 73.365 | 1.00 | 37.16 | 6 |
| ATOM | 18015 | C | LEU | G | 1169 | 59.166 | 90.055 | 76.060 | 1.00 | 24.69 | 6 |
| ATOM | 18016 | O | LEU | G | 1169 | 58.713 | 91.179 | 76.286 | 1.00 | 25.42 | 8 |
| ATOM | 18017 | N | GLU | G | 1170 | 59.316 | 89.125 | 76.997 | 1.00 | 47.21 | 7 |
| ATOM | 18018 | CA | GLU | G | 1170 | 58.950 | 89.344 | 78.390 | 1.00 | 48.70 | 6 |
| ATOM | 18019 | CB | GLU | G | 1170 | 58.909 | 88.003 | 79.128 | 1.00 | 167.86 | 6 |
| ATOM | 18020 | CG | GLU | G | 1170 | 58.327 | 88.046 | 80.535 | 1.00 | 172.42 | 6 |
| ATOM | 18021 | CD | GLU | G | 1170 | 56.883 | 88.509 | 80.559 | 1.00 | 175.08 | 6 |
| ATOM | 18022 | OE1 | GLU | G | 1170 | 56.131 | 88.185 | 79.615 | 1.00 | 176.00 | 8 |
| ATOM | 18023 | OE2 | GLU | G | 1170 | 56.496 | 89.186 | 81.533 | 1.00 | 178.08 | 8 |
| ATOM | 18024 | C | GLU | G | 1170 | 59.970 | 90.255 | 79.034 | 1.00 | 47.97 | 6 |
| ATOM | 18025 | O | GLU | G | 1170 | 59.609 | 91.177 | 79.758 | 1.00 | 48.43 | 8 |
| ATOM | 18026 | N | ASP | G | 1171 | 61.247 | 89.995 | 78.765 | 1.00 | 61.02 | 7 |
| ATOM | 18027 | CA | ASP | G | 1171 | 62.327 | 90.805 | 79.315 | 1.00 | 59.79 | 6 |
| ATOM | 18028 | CB | ASP | G | 1171 | 63.654 | 90.092 | 79.154 | 1.00 | 43.92 | 6 |
| ATOM | 18029 | CG | ASP | G | 1171 | 63.681 | 88.783 | 79.872 | 1.00 | 45.35 | 6 |
| ATOM | 18030 | OD1 | ASP | G | 1171 | 63.180 | 88.747 | 81.009 | 1.00 | 48.16 | 8 |
| ATOM | 18031 | OD2 | ASP | G | 1171 | 64.203 | 87.798 | 79.314 | 1.00 | 44.53 | 8 |
| ATOM | 18032 | C | ASP | G | 1171 | 62.405 | 92.147 | 78.624 | 1.00 | 58.13 | 6 |
| ATOM | 18033 | O | ASP | G | 1171 | 62.484 | 93.188 | 79.272 | 1.00 | 57.83 | 8 |
| ATOM | 18034 | N | VAL | G | 1172 | 62.400 | 92.112 | 77.298 | 1.00 | 42.43 | 7 |
| ATOM | 18035 | CA | VAL | G | 1172 | 62.435 | 93.329 | 76.503 | 1.00 | 41.13 | 6 |
| ATOM | 18036 | CB | VAL | G | 1172 | 62.234 | 93.013 | 75.021 | 1.00 | 21.88 | 6 |
| ATOM | 18037 | CG1 | VAL | G | 1172 | 62.201 | 94.305 | 74.209 | 1.00 | 19.66 | 6 |
| ATOM | 18038 | CG2 | VAL | G | 1172 | 63.343 | 92.079 | 74.554 | 1.00 | 22.09 | 6 |
| ATOM | 18039 | C | VAL | G | 1172 | 61.337 | 94.289 | 76.964 | 1.00 | 41.77 | 6 |
| ATOM | 18040 | O | VAL | G | 1172 | 61.308 | 95.460 | 76.581 | 1.00 | 43.44 | 8 |
| ATOM | 18041 | N | HIS | G | 1173 | 60.411 | 93.781 | 77.764 | 1.00 | 36.80 | 7 |
| ATOM | 18042 | CA | HIS | G | 1173 | 59.373 | 94.622 | 78.291 | 1.00 | 38.40 | 6 |
| ATOM | 18043 | CB | HIS | G | 1173 | 58.094 | 93.825 | 78.505 | 1.00 | 110.57 | 6 |
| ATOM | 18044 | CG | HIS | G | 1173 | 57.371 | 93.523 | 77.231 | 1.00 | 115.58 | 6 |
| ATOM | 18045 | CD2 | HIS | G | 1173 | 57.613 | 93.917 | 75.958 | 1.00 | 117.64 | 6 |
| ATOM | 18046 | ND1 | HIS | G | 1173 | 56.241 | 92.734 | 77.179 | 1.00 | 118.90 | 7 |
| ATOM | 18047 | CE1 | HIS | G | 1173 | 55.818 | 92.656 | 75.929 | 1.00 | 119.62 | 6 |
| ATOM | 18048 | NE2 | HIS | G | 1173 | 56.633 | 93.366 | 75.168 | 1.00 | 120.13 | 7 |
| ATOM | 18049 | C | HIS | G | 1173 | 59.961 | 95.131 | 79.591 | 1.00 | 37.73 | 6 |
| ATOM | 18050 | O | HIS | G | 1173 | 60.161 | 96.333 | 79.731 | 1.00 | 38.45 | 8 |
| ATOM | 18051 | N | PHE | G | 1174 | 60.292 | 94.229 | 80.517 | 1.00 | 55.21 | 7 |
| ATOM | 18052 | CA | PHE | G | 1174 | 60.880 | 94.645 | 81.797 | 1.00 | 53.36 | 6 |
| ATOM | 18053 | CB | PHE | G | 1174 | 61.608 | 93.478 | 82.479 | 1.00 | 44.66 | 6 |
| ATOM | 18054 | CG | PHE | G | 1174 | 62.478 | 93.888 | 83.665 | 1.00 | 42.45 | 6 |
| ATOM | 18055 | CD1 | PHE | G | 1174 | 61.984 | 94.721 | 84.667 | 1.00 | 41.72 | 6 |
| ATOM | 18056 | CD2 | PHE | G | 1174 | 63.780 | 93.401 | 83.794 | 1.00 | 40.90 | 6 |
| ATOM | 18057 | CE1 | PHE | G | 1174 | 62.772 | 95.058 | 85.776 | 1.00 | 39.75 | 6 |
| ATOM | 18058 | CE2 | PHE | G | 1174 | 64.566 | 93.735 | 84.898 | 1.00 | 39.02 | 6 |
| ATOM | 18059 | CZ | PHE | G | 1174 | 64.060 | 94.563 | 85.889 | 1.00 | 39.10 | 6 |
| ATOM | 18060 | C | PHE | G | 1174 | 61.857 | 95.787 | 81.569 | 1.00 | 53.30 | 6 |
| ATOM | 18061 | O | PHE | G | 1174 | 61.699 | 96.870 | 82.133 | 1.00 | 53.50 | 8 |
| ATOM | 18062 | N | LEU | G | 1175 | 62.863 | 95.541 | 80.739 | 1.00 | 27.60 | 7 |
| ATOM | 18063 | CA | LEU | G | 1175 | 63.838 | 96.565 | 80.433 | 1.00 | 26.26 | 6 |
| ATOM | 18064 | CB | LEU | G | 1175 | 64.746 | 96.094 | 79.313 | 1.00 | 15.95 | 6 |
| ATOM | 18065 | CG | LEU | G | 1175 | 65.808 | 95.147 | 79.835 | 1.00 | 14.06 | 6 |
| ATOM | 18066 | CD1 | LEU | G | 1175 | 66.453 | 94.418 | 78.695 | 1.00 | 15.14 | 6 |
| ATOM | 18067 | CD2 | LEU | G | 1175 | 66.827 | 95.938 | 80.618 | 1.00 | 13.87 | 6 |
| ATOM | 18068 | C | LEU | G | 1175 | 63.134 | 97.857 | 80.037 | 1.00 | 27.85 | 6 |
| ATOM | 18069 | O | LEU | G | 1175 | 63.455 | 98.926 | 80.539 | 1.00 | 28.80 | 8 |
| ATOM | 18070 | N | ILE | G | 1176 | 62.168 | 97.773 | 79.140 | 1.00 | 31.20 | 7 |
| ATOM | 18071 | CA | ILE | G | 1176 | 61.458 | 98.973 | 78.760 | 1.00 | 33.78 | 6 |
| ATOM | 18072 | CB | ILE | G | 1176 | 60.353 | 98.669 | 77.760 | 1.00 | 20.35 | 6 |
| ATOM | 18073 | CG2 | ILE | G | 1176 | 59.263 | 99.730 | 77.839 | 1.00 | 20.02 | 6 |
| ATOM | 18074 | CG1 | ILE | G | 1176 | 60.961 | 98.572 | 76.369 | 1.00 | 19.33 | 6 |
| ATOM | 18075 | CD1 | ILE | G | 1176 | 59.943 | 98.477 | 75.274 | 1.00 | 19.69 | 6 |
| ATOM | 18076 | C | ILE | G | 1176 | 60.840 | 99.643 | 79.978 | 1.00 | 36.85 | 6 |
| ATOM | 18077 | O | ILE | G | 1176 | 60.769 | 100.866 | 80.039 | 1.00 | 36.89 | 8 |
| ATOM | 18078 | N | LYS | G | 1177 | 60.384 | 98.855 | 80.945 | 1.00 | 71.25 | 7 |
| ATOM | 18079 | CA | LYS | G | 1177 | 59.776 | 99.440 | 82.131 | 1.00 | 74.99 | 6 |
| ATOM | 18080 | CB | LYS | G | 1177 | 58.807 | 98.459 | 82.798 | 1.00 | 132.30 | 6 |
| ATOM | 18081 | CG | LYS | G | 1177 | 57.395 | 98.552 | 82.234 | 1.00 | 135.70 | 6 |
| ATOM | 18082 | CD | LYS | G | 1177 | 56.434 | 97.558 | 82.872 | 1.00 | 137.66 | 6 |
| ATOM | 18083 | CE | LYS | G | 1177 | 55.035 | 97.695 | 82.264 | 1.00 | 139.50 | 6 |
| ATOM | 18084 | NZ | LYS | G | 1177 | 54.046 | 96.696 | 82.776 | 1.00 | 138.30 | 7 |
| ATOM | 18085 | C | LYS | G | 1177 | 60.811 | 99.916 | 83.122 | 1.00 | 76.49 | 6 |
| ATOM | 18086 | O | LYS | G | 1177 | 60.581 | 100.886 | 83.837 | 1.00 | 78.61 | 8 |
| ATOM | 18087 | N | ALA | G | 1178 | 61.956 | 99.244 | 83.161 | 1.00 | 51.20 | 7 |
| ATOM | 18088 | CA | ALA | G | 1178 | 63.025 | 99.641 | 84.076 | 1.00 | 51.49 | 6 |
| ATOM | 18089 | CB | ALA | G | 1178 | 64.124 | 98.581 | 84.098 | 1.00 | 19.49 | 6 |
| ATOM | 18090 | C | ALA | G | 1178 | 63.584 | 100.985 | 83.612 | 1.00 | 51.85 | 6 |
| ATOM | 18091 | O | ALA | G | 1178 | 63.987 | 101.822 | 84.414 | 1.00 | 51.20 | 8 |
| ATOM | 18092 | N | ALA | G | 1179 | 63.586 | 101.186 | 82.301 | 1.00 | 45.86 | 7 |
| ATOM | 18093 | CA | ALA | G | 1179 | 64.076 | 102.422 | 81.714 | 1.00 | 47.10 | 6 |
| ATOM | 18094 | CB | ALA | G | 1179 | 64.370 | 102.216 | 80.231 | 1.00 | 141.45 | 6 |
| ATOM | 18095 | C | ALA | G | 1179 | 63.054 | 103.542 | 81.899 | 1.00 | 48.32 | 6 |
| ATOM | 18096 | O | ALA | G | 1179 | 63.306 | 104.690 | 81.515 | 1.00 | 50.16 | 8 |
| ATOM | 18097 | N | GLU | G | 1180 | 61.894 | 103.202 | 82.456 | 1.00 | 44.25 | 7 |
| ATOM | 18098 | CA | GLU | G | 1180 | 60.862 | 104.197 | 82.726 | 1.00 | 44.57 | 6 |
| ATOM | 18099 | CB | GLU | G | 1180 | 59.468 | 103.630 | 82.487 | 1.00 | 76.96 | 6 |
| ATOM | 18100 | CG | GLU | G | 1180 | 59.144 | 103.378 | 81.038 | 1.00 | 79.70 | 6 |
| ATOM | 18101 | CD | GLU | G | 1180 | 57.669 | 103.132 | 80.832 | 1.00 | 81.71 | 6 |
| ATOM | 18102 | OE1 | GLU | G | 1180 | 57.091 | 102.348 | 81.616 | 1.00 | 83.04 | 8 |
| ATOM | 18103 | OE2 | GLU | G | 1180 | 57.086 | 103.713 | 79.892 | 1.00 | 82.50 | 8 |
| ATOM | 18104 | C | GLU | G | 1180 | 61.034 | 104.567 | 84.194 | 1.00 | 43.84 | 6 |
| ATOM | 18105 | O | GLU | G | 1180 | 61.014 | 105.741 | 84.576 | 1.00 | 44.27 | 8 |
| ATOM | 18106 | N | ALA | G | 1181 | 61.209 | 103.554 | 85.027 | 1.00 | 74.61 | 7 |
| ATOM | 18107 | CA | ALA | G | 1181 | 61.437 | 103.815 | 86.431 | 1.00 | 74.64 | 6 |
| ATOM | 18108 | CB | ALA | G | 1181 | 61.754 | 102.521 | 87.160 | 1.00 | 98.13 | 6 |
| ATOM | 18109 | C | ALA | G | 1181 | 62.656 | 104.730 | 86.423 | 1.00 | 74.18 | 6 |
| ATOM | 18110 | O | ALA | G | 1181 | 62.783 | 105.625 | 87.248 | 1.00 | 75.56 | 8 |
| ATOM | 18111 | N | GLY | G | 1182 | 63.535 | 104.502 | 85.450 | 1.00 | 40.36 | 7 |
| ATOM | 18112 | CA | GLY | G | 1182 | 64.745 | 105.287 | 85.323 | 1.00 | 38.14 | 6 |
| ATOM | 18113 | C | GLY | G | 1182 | 65.882 | 104.546 | 85.986 | 1.00 | 37.13 | 6 |
| ATOM | 18114 | O | GLY | G | 1182 | 66.362 | 104.972 | 87.031 | 1.00 | 38.37 | 8 |
| ATOM | 18115 | N | GLU | G | 1183 | 66.295 | 103.423 | 85.393 | 1.00 | 53.62 | 7 |
| ATOM | 18116 | CA | GLU | G | 1183 | 67.394 | 102.608 | 85.924 | 1.00 | 52.00 | 6 |
| ATOM | 18117 | CB | GLU | G | 1183 | 66.843 | 101.406 | 86.707 | 1.00 | 61.14 | 6 |
| ATOM | 18118 | CG | GLU | G | 1183 | 67.517 | 101.211 | 88.064 | 1.00 | 61.51 | 6 |
| ATOM | 18119 | CD | GLU | G | 1183 | 66.596 | 100.603 | 89.133 | 1.00 | 63.38 | 6 |
| ATOM | 18120 | OE1 | GLU | G | 1183 | 65.376 | 100.891 | 89.116 | 1.00 | 64.65 | 8 |
| ATOM | 18121 | OE2 | GLU | G | 1183 | 67.099 | 99.860 | 90.012 | 1.00 | 62.63 | 8 |
| ATOM | 18122 | C | GLU | G | 1183 | 68.365 | 102.137 | 84.834 | 1.00 | 50.71 | 6 |
| ATOM | 18123 | O | GLU | G | 1183 | 69.439 | 101.633 | 85.149 | 1.00 | 50.20 | 8 |
| ATOM | 18124 | N | VAL | G | 1184 | 67.991 | 102.300 | 83.561 | 1.00 | 64.02 | 7 |
| ATOM | 18125 | CA | VAL | G | 1184 | 68.859 | 101.922 | 82.432 | 1.00 | 62.35 | 6 |
| ATOM | 18126 | CB | VAL | G | 1184 | 68.683 | 100.452 | 82.019 | 1.00 | 61.39 | 6 |
| ATOM | 18127 | CG1 | VAL | G | 1184 | 69.889 | 100.011 | 81.190 | 1.00 | 61.64 | 6 |
| ATOM | 18128 | CG2 | VAL | G | 1184 | 68.516 | 99.580 | 83.230 | 1.00 | 61.82 | 6 |
| ATOM | 18129 | C | VAL | G | 1184 | 68.566 | 102.760 | 81.180 | 1.00 | 61.52 | 6 |
| ATOM | 18130 | O | VAL | G | 1184 | 67.415 | 103.137 | 80.950 | 1.00 | 62.38 | 8 |
| ATOM | 18131 | N | ARG | G | 1185 | 69.591 | 103.033 | 80.365 | 1.00 | 23.18 | 7 |
| ATOM | 18132 | CA | ARG | G | 1185 | 69.404 | 103.823 | 79.138 | 1.00 | 21.35 | 6 |
| ATOM | 18133 | CB | ARG | G | 1185 | 70.027 | 105.200 | 79.305 | 1.00 | 34.14 | 6 |
| ATOM | 18134 | CG | ARG | G | 1185 | 69.225 | 106.141 | 80.150 | 1.00 | 32.31 | 6 |
| ATOM | 18135 | CD | ARG | G | 1185 | 69.382 | 107.535 | 79.598 | 1.00 | 31.73 | 6 |
| ATOM | 18136 | NE | ARG | G | 1185 | 68.282 | 108.409 | 79.976 | 1.00 | 30.59 | 7 |
| ATOM | 18137 | CZ | ARG | G | 1185 | 67.977 | 109.523 | 79.325 | 1.00 | 29.80 | 6 |
| ATOM | 18138 | NH1 | ARG | G | 1185 | 68.700 | 109.880 | 78.276 | 1.00 | 29.91 | 7 |
| ATOM | 18139 | NH2 | ARG | G | 1185 | 66.936 | 110.259 | 79.698 | 1.00 | 29.68 | 7 |
| ATOM | 18140 | C | ARG | G | 1185 | 69.946 | 103.155 | 77.852 | 1.00 | 21.54 | 6 |
| ATOM | 18141 | O | ARG | G | 1185 | 69.406 | 103.337 | 76.746 | 1.00 | 20.03 | 8 |
| ATOM | 18142 | N | GLU | G | 1186 | 71.037 | 102.414 | 77.990 | 1.00 | 46.36 | 7 |
| ATOM | 18143 | CA | GLU | G | 1186 | 71.595 | 101.683 | 76.868 | 1.00 | 48.61 | 6 |
| ATOM | 18144 | CB | GLU | G | 1186 | 73.061 | 102.036 | 76.637 | 1.00 | 150.56 | 6 |
| ATOM | 18145 | CG | GLU | G | 1186 | 73.329 | 103.478 | 76.279 | 1.00 | 156.13 | 6 |
| ATOM | 18146 | CD | GLU | G | 1186 | 74.740 | 103.678 | 75.749 | 1.00 | 160.61 | 6 |
| ATOM | 18147 | OE1 | GLU | G | 1186 | 75.000 | 103.295 | 74.587 | 1.00 | 161.94 | 8 |
| ATOM | 18148 | OE2 | GLU | G | 1186 | 75.595 | 104.200 | 76.498 | 1.00 | 164.12 | 8 |
| ATOM | 18149 | C | GLU | G | 1186 | 71.489 | 100.246 | 77.366 | 1.00 | 48.64 | 6 |
| ATOM | 18150 | O | GLU | G | 1186 | 71.292 | 100.027 | 78.568 | 1.00 | 48.96 | 8 |
| ATOM | 18151 | N | VAL | G | 1187 | 71.599 | 99.276 | 76.459 | 1.00 | 27.64 | 7 |
| ATOM | 18152 | CA | VAL | G | 1187 | 71.528 | 97.868 | 76.834 | 1.00 | 26.21 | 6 |
| ATOM | 18153 | CB | VAL | G | 1187 | 70.191 | 97.206 | 76.378 | 1.00 | 17.99 | 6 |
| ATOM | 18154 | CG1 | VAL | G | 1187 | 70.149 | 95.776 | 76.856 | 1.00 | 16.19 | 6 |
| ATOM | 18155 | CG2 | VAL | G | 1187 | 68.976 | 97.966 | 76.938 | 1.00 | 15.76 | 6 |
| ATOM | 18156 | C | VAL | G | 1187 | 72.695 | 97.145 | 76.177 | 1.00 | 27.11 | 6 |
| ATOM | 18157 | O | VAL | G | 1187 | 72.805 | 97.145 | 74.964 | 1.00 | 26.72 | 8 |
| ATOM | 18158 | N | PRO | G | 1188 | 73.584 | 96.528 | 76.981 | 1.00 | 45.28 | 7 |
| ATOM | 18159 | CD | PRO | G | 1188 | 73.422 | 96.413 | 78.441 | 1.00 | 87.43 | 6 |
| ATOM | 18160 | CA | PRO | G | 1188 | 74.775 | 95.782 | 76.555 | 1.00 | 45.41 | 6 |
| ATOM | 18161 | CB | PRO | G | 1188 | 75.516 | 95.562 | 77.860 | 1.00 | 87.26 | 6 |
| ATOM | 18162 | CG | PRO | G | 1188 | 74.399 | 95.298 | 78.794 | 1.00 | 88.03 | 6 |
| ATOM | 18163 | C | PRO | G | 1188 | 74.384 | 94.463 | 75.909 | 1.00 | 45.11 | 6 |
| ATOM | 18164 | O | PRO | G | 1188 | 74.324 | 93.429 | 76.577 | 1.00 | 44.27 | 8 |
| ATOM | 18165 | N | VAL | G | 1189 | 74.147 | 94.505 | 74.602 | 1.00 | 18.81 | 7 |
| ATOM | 18166 | CA | VAL | G | 1189 | 73.715 | 93.336 | 73.857 | 1.00 | 19.53 | 6 |
| ATOM | 18167 | CB | VAL | G | 1189 | 72.255 | 93.551 | 73.388 | 1.00 | 54.34 | 6 |
| ATOM | 18168 | CG1 | VAL | G | 1189 | 72.157 | 93.687 | 71.880 | 1.00 | 53.73 | 6 |
| ATOM | 18169 | CG2 | VAL | G | 1189 | 71.418 | 92.442 | 73.904 | 1.00 | 56.33 | 6 |
| ATOM | 18170 | C | VAL | G | 1189 | 74.637 | 93.034 | 72.682 | 1.00 | 20.15 | 6 |
| ATOM | 18171 | O | VAL | G | 1189 | 75.028 | 93.939 | 71.941 | 1.00 | 19.21 | 8 |
| ATOM | 18172 | N | ARG | G | 1190 | 74.977 | 91.753 | 72.528 | 1.00 | 39.78 | 7 |
| ATOM | 18173 | CA | ARG | G | 1190 | 75.869 | 91.278 | 71.467 | 1.00 | 42.42 | 6 |
| ATOM | 18174 | CB | ARG | G | 1190 | 76.465 | 89.937 | 71.876 | 1.00 | 72.84 | 6 |
| ATOM | 18175 | CG | ARG | G | 1190 | 77.099 | 89.978 | 73.240 | 1.00 | 75.85 | 6 |
| ATOM | 18176 | CD | ARG | G | 1190 | 78.285 | 89.050 | 73.318 | 1.00 | 77.83 | 6 |
| ATOM | 18177 | NE | ARG | G | 1190 | 77.888 | 87.651 | 73.252 | 1.00 | 79.70 | 7 |
| ATOM | 18178 | CZ | ARG | G | 1190 | 78.735 | 86.647 | 73.047 | 1.00 | 81.18 | 6 |
| ATOM | 18179 | NH1 | ARG | G | 1190 | 80.030 | 86.887 | 72.881 | 1.00 | 80.55 | 7 |
| ATOM | 18180 | NH2 | ARG | G | 1190 | 78.287 | 85.399 | 73.018 | 1.00 | 81.76 | 7 |
| ATOM | 18181 | C | ARG | G | 1190 | 75.189 | 91.147 | 70.102 | 1.00 | 43.65 | 6 |
| ATOM | 18182 | O | ARG | G | 1190 | 74.259 | 90.368 | 69.935 | 1.00 | 44.86 | 8 |
| ATOM | 18183 | N | SER | G | 1191 | 75.677 | 91.889 | 69.118 | 1.00 | 49.48 | 7 |
| ATOM | 18184 | CA | SER | G | 1191 | 75.079 | 91.870 | 67.792 | 1.00 | 51.25 | 6 |
| ATOM | 18185 | CB | SER | G | 1191 | 74.389 | 93.219 | 67.564 | 1.00 | 46.33 | 6 |
| ATOM | 18186 | OG | SER | G | 1191 | 74.207 | 93.505 | 66.192 | 1.00 | 46.50 | 8 |
| ATOM | 18187 | C | SER | G | 1191 | 76.072 | 91.569 | 66.661 | 1.00 | 52.86 | 6 |
| ATOM | 18188 | O | SER | G | 1191 | 77.272 | 91.702 | 66.841 | 1.00 | 52.77 | 8 |
| ATOM | 18189 | N | PRO | G | 1192 | 75.573 | 91.132 | 65.487 | 1.00 | 39.77 | 7 |
| ATOM | 18190 | CD | PRO | G | 1192 | 74.166 | 90.756 | 65.249 | 1.00 | 52.04 | 6 |
| ATOM | 18191 | CA | PRO | G | 1192 | 76.383 | 90.807 | 64.310 | 1.00 | 40.60 | 6 |
| ATOM | 18192 | CB | PRO | G | 1192 | 75.331 | 90.544 | 63.240 | 1.00 | 51.16 | 6 |
| ATOM | 18193 | CG | PRO | G | 1192 | 74.261 | 89.888 | 64.012 | 1.00 | 51.67 | 6 |
| ATOM | 18194 | C | PRO | G | 1192 | 77.342 | 91.920 | 63.896 | 1.00 | 42.52 | 6 |
| ATOM | 18195 | O | PRO | G | 1192 | 78.437 | 91.646 | 63.389 | 1.00 | 43.76 | 8 |
| ATOM | 18196 | N | LEU | G | 1193 | 76.935 | 93.172 | 64.101 | 1.00 | 67.69 | 7 |
| ATOM | 18197 | CA | LEU | G | 1193 | 77.780 | 94.302 | 63.732 | 1.00 | 70.26 | 6 |
| ATOM | 18198 | CB | LEU | G | 1193 | 77.117 | 95.619 | 64.126 | 1.00 | 70.36 | 6 |
| ATOM | 18199 | CG | LEU | G | 1193 | 75.956 | 96.075 | 63.247 | 1.00 | 71.83 | 6 |
| ATOM | 18200 | CD1 | LEU | G | 1193 | 76.070 | 97.571 | 62.996 | 1.00 | 72.04 | 6 |
| ATOM | 18201 | CD2 | LEU | G | 1193 | 75.991 | 95.340 | 61.929 | 1.00 | 72.96 | 6 |
| ATOM | 18202 | C | LEU | G | 1193 | 79.173 | 94.229 | 64.351 | 1.00 | 71.54 | 6 |
| ATOM | 18203 | O | LEU | G | 1193 | 80.123 | 94.840 | 63.848 | 1.00 | 72.01 | 8 |
| ATOM | 18204 | N | THR | G | 1194 | 79.283 | 93.471 | 65.439 | 1.00 | 28.21 | 7 |
| ATOM | 18205 | CA | THR | G | 1194 | 80.535 | 93.293 | 66.151 | 1.00 | 29.10 | 6 |
| ATOM | 18206 | CB | THR | G | 1194 | 80.478 | 93.960 | 67.543 | 1.00 | 92.72 | 6 |
| ATOM | 18207 | OG1 | THR | G | 1194 | 81.616 | 93.553 | 68.309 | 1.00 | 95.37 | 8 |
| ATOM | 18208 | CG2 | THR | G | 1194 | 79.221 | 93.559 | 68.289 | 1.00 | 94.01 | 6 |
| ATOM | 18209 | C | THR | G | 1194 | 80.908 | 91.825 | 66.344 | 1.00 | 29.40 | 6 |
| ATOM | 18210 | O | THR | G | 1194 | 80.981 | 91.354 | 67.475 | 1.00 | 29.07 | 8 |
| ATOM | 18211 | N | CYS | G | 1195 | 81.156 | 91.095 | 65.261 | 1.00 | 109.03 | 7 |
| ATOM | 18212 | CA | CYS | G | 1195 | 81.532 | 89.702 | 65.437 | 1.00 | 111.64 | 6 |
| ATOM | 18213 | CB | CYS | G | 1195 | 80.381 | 88.790 | 65.059 | 1.00 | 54.80 | 6 |
| ATOM | 18214 | SG | CYS | G | 1195 | 80.333 | 87.402 | 66.156 | 1.00 | 51.20 | 16 |
| ATOM | 18215 | C | CYS | G | 1195 | 82.818 | 89.263 | 64.742 | 1.00 | 114.47 | 6 |
| ATOM | 18216 | O | CYS | G | 1195 | 83.103 | 89.646 | 63.606 | 1.00 | 115.34 | 8 |
| ATOM | 18217 | N | ALA | G | 1196 | 83.576 | 88.434 | 65.456 | 1.00 | 107.79 | 7 |
| ATOM | 18218 | CA | ALA | G | 1196 | 84.879 | 87.946 | 65.025 | 1.00 | 111.57 | 6 |
| ATOM | 18219 | CB | ALA | G | 1196 | 85.535 | 87.197 | 66.177 | 1.00 | 113.65 | 6 |
| ATOM | 18220 | C | ALA | G | 1196 | 84.998 | 87.118 | 63.749 | 1.00 | 113.95 | 6 |
| ATOM | 18221 | O | ALA | G | 1196 | 86.112 | 86.851 | 63.294 | 1.00 | 114.22 | 8 |
| ATOM | 18222 | N | THR | G | 1197 | 83.886 | 86.708 | 63.156 | 1.00 | 109.64 | 7 |
| ATOM | 18223 | CA | THR | G | 1197 | 83.993 | 85.914 | 61.939 | 1.00 | 111.92 | 6 |
| ATOM | 18224 | CB | THR | G | 1197 | 83.339 | 84.533 | 62.130 | 1.00 | 103.22 | 6 |
| ATOM | 18225 | OG1 | THR | G | 1197 | 83.339 | 84.189 | 63.521 | 1.00 | 103.44 | 8 |
| ATOM | 18226 | CG2 | THR | G | 1197 | 84.112 | 83.471 | 61.351 | 1.00 | 103.53 | 6 |
| ATOM | 18227 | C | THR | G | 1197 | 83.353 | 86.609 | 60.734 | 1.00 | 113.33 | 6 |
| ATOM | 18228 | O | THR | G | 1197 | 82.145 | 86.493 | 60.525 | 1.00 | 113.81 | 8 |
| ATOM | 18229 | N | ALA | G | 1198 | 84.156 | 87.327 | 59.944 | 1.00 | 208.87 | 7 |
| ATOM | 18230 | CA | ALA | G | 1198 | 83.647 | 88.030 | 58.761 | 1.00 | 208.87 | 6 |
| ATOM | 18231 | CB | ALA | G | 1198 | 84.792 | 88.716 | 58.006 | 1.00 | 46.78 | 6 |
| ATOM | 18232 | C | ALA | G | 1198 | 82.932 | 87.051 | 57.838 | 1.00 | 208.87 | 6 |
| ATOM | 18233 | O | ALA | G | 1198 | 81.749 | 86.764 | 58.018 | 1.00 | 208.87 | 8 |
| ATOM | 18234 | N | TYR | G | 1199 | 83.658 | 86.546 | 56.847 | 1.00 | 108.37 | 7 |
| ATOM | 18235 | CA | TYR | G | 1199 | 83.104 | 85.585 | 55.900 | 1.00 | 108.99 | 6 |
| ATOM | 18236 | CB | TYR | G | 1199 | 84.244 | 84.780 | 55.264 | 1.00 | 88.96 | 6 |
| ATOM | 18237 | CG | TYR | G | 1199 | 85.055 | 85.582 | 54.270 | 1.00 | 90.45 | 6 |
| ATOM | 18238 | CD1 | TYR | G | 1199 | 86.376 | 85.252 | 53.986 | 1.00 | 91.36 | 6 |
| ATOM | 18239 | CE1 | TYR | G | 1199 | 87.127 | 86.001 | 53.074 | 1.00 | 92.40 | 6 |
| ATOM | 18240 | CD2 | TYR | G | 1199 | 84.496 | 86.679 | 53.615 | 1.00 | 90.54 | 6 |
| ATOM | 18241 | CE2 | TYR | G | 1199 | 85.232 | 87.433 | 52.703 | 1.00 | 91.49 | 6 |
| ATOM | 18242 | CZ | TYR | G | 1199 | 86.549 | 87.094 | 52.437 | 1.00 | 92.07 | 6 |
| ATOM | 18243 | OH | TYR | G | 1199 | 87.279 | 87.864 | 51.551 | 1.00 | 91.83 | 8 |
| ATOM | 18244 | C | TYR | G | 1199 | 82.094 | 84.658 | 56.574 | 1.00 | 108.81 | 6 |
| ATOM | 18245 | O | TYR | G | 1199 | 80.979 | 84.493 | 56.096 | 1.00 | 108.02 | 8 |
| ATOM | 18246 | N | GLY | G | 1200 | 82.474 | 84.061 | 57.693 | 1.00 | 208.87 | 7 |
| ATOM | 18247 | CA | GLY | G | 1200 | 81.537 | 83.193 | 58.375 | 1.00 | 208.87 | 6 |
| ATOM | 18248 | C | GLY | G | 1200 | 80.714 | 83.986 | 59.374 | 1.00 | 208.87 | 6 |
| ATOM | 18249 | O | GLY | G | 1200 | 81.183 | 84.251 | 60.476 | 1.00 | 208.87 | 8 |
| ATOM | 18250 | N | ALA | G | 1201 | 79.498 | 84.377 | 59.000 | 1.00 | 97.50 | 7 |
| ATOM | 18251 | CA | ALA | G | 1201 | 78.619 | 85.137 | 59.898 | 1.00 | 96.17 | 6 |
| ATOM | 18252 | CB | ALA | G | 1201 | 77.201 | 85.064 | 59.414 | 1.00 | 140.38 | 6 |
| ATOM | 18253 | C | ALA | G | 1201 | 78.688 | 84.602 | 61.319 | 1.00 | 95.56 | 6 |
| ATOM | 18254 | O | ALA | G | 1201 | 79.069 | 83.456 | 61.542 | 1.00 | 95.15 | 8 |
| ATOM | 18255 | N | CYS | G | 1202 | 78.297 | 85.408 | 62.294 | 1.00 | 66.28 | 7 |
| ATOM | 18256 | CA | CYS | G | 1202 | 78.387 | 84.907 | 63.648 | 1.00 | 65.50 | 6 |
| ATOM | 18257 | CB | CYS | G | 1202 | 78.072 | 85.978 | 64.679 | 1.00 | 208.87 | 6 |
| ATOM | 18258 | SG | CYS | G | 1202 | 78.241 | 85.327 | 66.366 | 1.00 | 208.87 | 16 |
| ATOM | 18259 | C | CYS | G | 1202 | 77.439 | 83.760 | 63.853 | 1.00 | 62.81 | 6 |
| ATOM | 18260 | O | CYS | G | 1202 | 76.628 | 83.460 | 62.984 | 1.00 | 62.33 | 8 |
| ATOM | 18261 | N | ALA | G | 1203 | 77.556 | 83.129 | 65.019 | 1.00 | 126.53 | 7 |
| ATOM | 18262 | CA | ALA | G | 1203 | 76.720 | 82.003 | 65.408 | 1.00 | 123.45 | 6 |
| ATOM | 18263 | CB | ALA | G | 1203 | 77.449 | 80.720 | 65.154 | 1.00 | 102.53 | 6 |
| ATOM | 18264 | C | ALA | G | 1203 | 76.370 | 82.117 | 66.885 | 1.00 | 120.96 | 6 |
| ATOM | 18265 | O | ALA | G | 1203 | 77.095 | 81.614 | 67.739 | 1.00 | 121.39 | 8 |
| ATOM | 18266 | N | LYS | G | 1204 | 75.249 | 82.781 | 67.151 | 1.00 | 51.38 | 7 |
| ATOM | 18267 | CA | LYS | G | 1204 | 74.713 | 83.041 | 68.488 | 1.00 | 47.97 | 6 |
| ATOM | 18268 | CB | LYS | G | 1204 | 75.282 | 82.065 | 69.520 | 1.00 | 134.40 | 6 |
| ATOM | 18269 | CG | LYS | G | 1204 | 74.639 | 80.688 | 69.465 | 1.00 | 136.46 | 6 |
| ATOM | 18270 | CD | LYS | G | 1204 | 74.859 | 79.950 | 70.776 | 1.00 | 136.68 | 6 |
| ATOM | 18271 | CE | LYS | G | 1204 | 73.938 | 78.748 | 70.918 | 1.00 | 136.14 | 6 |
| ATOM | 18272 | NZ | LYS | G | 1204 | 74.038 | 78.143 | 72.282 | 1.00 | 134.86 | 7 |
| ATOM | 18273 | C | LYS | G | 1204 | 74.897 | 84.494 | 68.967 | 1.00 | 43.96 | 6 |
| ATOM | 18274 | O | LYS | G | 1204 | 74.990 | 84.770 | 70.164 | 1.00 | 44.01 | 8 |
| ATOM | 18275 | N | CYS | G | 1205 | 74.984 | 85.410 | 68.010 | 1.00 | 66.03 | 7 |
| ATOM | 18276 | CA | CYS | G | 1205 | 75.077 | 86.841 | 68.282 | 1.00 | 61.43 | 6 |
| ATOM | 18277 | CB | CYS | G | 1205 | 76.322 | 87.476 | 67.661 | 1.00 | 68.90 | 6 |
| ATOM | 18278 | SG | CYS | G | 1205 | 77.842 | 87.350 | 68.644 | 1.00 | 66.16 | 16 |
| ATOM | 18279 | C | CYS | G | 1205 | 73.835 | 87.279 | 67.525 | 1.00 | 59.10 | 6 |
| ATOM | 18280 | O | CYS | G | 1205 | 73.209 | 88.299 | 67.816 | 1.00 | 60.26 | 8 |
| ATOM | 18281 | N | TYR | G | 1206 | 73.487 | 86.466 | 66.537 | 1.00 | 66.00 | 7 |
| ATOM | 18282 | CA | TYR | G | 1206 | 72.294 | 86.676 | 65.750 | 1.00 | 61.13 | 6 |
| ATOM | 18283 | CB | TYR | G | 1206 | 72.423 | 85.935 | 64.420 | 1.00 | 58.42 | 6 |
| ATOM | 18284 | CG | TYR | G | 1206 | 71.150 | 85.884 | 63.617 | 1.00 | 59.28 | 6 |
| ATOM | 18285 | CD1 | TYR | G | 1206 | 70.737 | 86.966 | 62.853 | 1.00 | 60.28 | 6 |
| ATOM | 18286 | CE1 | TYR | G | 1206 | 69.538 | 86.930 | 62.155 | 1.00 | 61.02 | 6 |
| ATOM | 18287 | CD2 | TYR | G | 1206 | 70.334 | 84.766 | 63.658 | 1.00 | 60.32 | 6 |
| ATOM | 18288 | CE2 | TYR | G | 1206 | 69.139 | 84.720 | 62.969 | 1.00 | 60.55 | 6 |
| ATOM | 18289 | CZ | TYR | G | 1206 | 68.745 | 85.801 | 62.222 | 1.00 | 61.51 | 6 |
| ATOM | 18290 | OH | TYR | G | 1206 | 67.547 | 85.761 | 61.553 | 1.00 | 61.96 | 8 |
| ATOM | 18291 | C | TYR | G | 1206 | 71.229 | 86.026 | 66.633 | 1.00 | 57.67 | 6 |
| ATOM | 18292 | O | TYR | G | 1206 | 70.102 | 85.804 | 66.206 | 1.00 | 58.77 | 8 |
| ATOM | 18293 | N | GLY | G | 1207 | 71.637 | 85.723 | 67.869 | 1.00 | 28.17 | 7 |
| ATOM | 18294 | CA | GLY | G | 1207 | 70.801 | 85.100 | 68.888 | 1.00 | 23.00 | 6 |
| ATOM | 18295 | C | GLY | G | 1207 | 69.319 | 84.936 | 68.629 | 1.00 | 20.12 | 6 |
| ATOM | 18296 | O | GLY | G | 1207 | 68.665 | 85.809 | 68.069 | 1.00 | 19.50 | 8 |
| ATOM | 18297 | N | TYR | G | 1208 | 68.772 | 83.810 | 69.059 | 1.00 | 16.34 | 7 |
| ATOM | 18298 | CA | TYR | G | 1208 | 67.359 | 83.541 | 68.848 | 1.00 | 13.99 | 6 |
| ATOM | 18299 | CB | TYR | G | 1208 | 66.497 | 84.669 | 69.422 | 1.00 | 27.53 | 6 |
| ATOM | 18300 | CG | TYR | G | 1208 | 65.642 | 84.289 | 70.614 | 1.00 | 25.27 | 6 |
| ATOM | 18301 | CD1 | TYR | G | 1208 | 64.255 | 84.322 | 70.536 | 1.00 | 25.01 | 6 |
| ATOM | 18302 | CE1 | TYR | G | 1208 | 63.469 | 84.034 | 71.636 | 1.00 | 25.68 | 6 |
| ATOM | 18303 | CD2 | TYR | G | 1208 | 66.222 | 83.948 | 71.831 | 1.00 | 24.20 | 6 |
| ATOM | 18304 | CE2 | TYR | G | 1208 | 65.448 | 83.653 | 72.943 | 1.00 | 25.21 | 6 |
| ATOM | 18305 | CZ | TYR | G | 1208 | 64.071 | 83.698 | 72.838 | 1.00 | 26.65 | 6 |
| ATOM | 18306 | OH | TYR | G | 1208 | 63.299 | 83.399 | 73.941 | 1.00 | 28.05 | 8 |
| ATOM | 18307 | C | TYR | G | 1208 | 67.054 | 83.405 | 67.365 | 1.00 | 13.87 | 6 |
| ATOM | 18308 | O | TYR | G | 1208 | 67.193 | 84.361 | 66.603 | 1.00 | 13.87 | 8 |
| ATOM | 18309 | N | ASP | G | 1209 | 66.647 | 82.200 | 66.970 | 1.00 | 43.28 | 7 |
| ATOM | 18310 | CA | ASP | G | 1209 | 66.262 | 81.898 | 65.591 | 1.00 | 42.53 | 6 |
| ATOM | 18311 | CB | ASP | G | 1209 | 65.555 | 80.544 | 65.536 | 1.00 | 31.74 | 6 |
| ATOM | 18312 | CG | ASP | G | 1209 | 65.062 | 80.197 | 64.151 | 1.00 | 31.62 | 6 |
| ATOM | 18313 | OD1 | ASP | G | 1209 | 64.384 | 81.039 | 63.523 | 1.00 | 31.04 | 8 |
| ATOM | 18314 | OD2 | ASP | G | 1209 | 65.350 | 79.070 | 63.694 | 1.00 | 34.00 | 8 |
| ATOM | 18315 | C | ASP | G | 1209 | 65.290 | 82.977 | 65.133 | 1.00 | 42.29 | 6 |
| ATOM | 18316 | O | ASP | G | 1209 | 65.181 | 83.278 | 63.944 | 1.00 | 43.52 | 8 |
| ATOM | 18317 | N | LEU | G | 1210 | 64.572 | 83.533 | 66.101 | 1.00 | 29.19 | 7 |
| ATOM | 18318 | CA | LEU | G | 1210 | 63.610 | 84.587 | 65.858 | 1.00 | 27.76 | 6 |
| ATOM | 18319 | CB | LEU | G | 1210 | 64.260 | 85.700 | 65.039 | 1.00 | 16.20 | 6 |
| ATOM | 18320 | CG | LEU | G | 1210 | 63.420 | 86.960 | 64.945 | 1.00 | 15.40 | 6 |
| ATOM | 18321 | CD1 | LEU | G | 1210 | 63.091 | 87.431 | 66.337 | 1.00 | 15.41 | 6 |
| ATOM | 18322 | CD2 | LEU | G | 1210 | 64.173 | 88.022 | 64.168 | 1.00 | 16.19 | 6 |
| ATOM | 18323 | C | LEU | G | 1210 | 62.345 | 84.080 | 65.160 | 1.00 | 27.63 | 6 |
| ATOM | 18324 | O | LEU | G | 1210 | 61.424 | 84.866 | 64.865 | 1.00 | 26.89 | 8 |
| ATOM | 18325 | N | SER | G | 1211 | 62.298 | 82.773 | 64.903 | 1.00 | 27.94 | 7 |
| ATOM | 18326 | CA | SER | G | 1211 | 61.137 | 82.176 | 64.250 | 1.00 | 27.58 | 6 |
| ATOM | 18327 | CB | SER | G | 1211 | 61.464 | 81.704 | 62.842 | 1.00 | 24.99 | 6 |
| ATOM | 18328 | OG | SER | G | 1211 | 61.427 | 80.288 | 62.798 | 1.00 | 21.95 | 8 |
| ATOM | 18329 | C | SER | G | 1211 | 60.660 | 80.987 | 65.050 | 1.00 | 28.55 | 6 |
| ATOM | 18330 | O | SER | G | 1211 | 59.543 | 80.511 | 64.865 | 1.00 | 27.88 | 8 |
| ATOM | 18331 | N | MET | G | 1212 | 61.520 | 80.489 | 65.924 | 1.00 | 57.72 | 7 |
| ATOM | 18332 | CA | MET | G | 1212 | 61.145 | 79.369 | 66.761 | 1.00 | 60.29 | 6 |
| ATOM | 18333 | CB | MET | G | 1212 | 62.089 | 78.182 | 66.538 | 1.00 | 49.27 | 6 |
| ATOM | 18334 | CG | MET | G | 1212 | 62.027 | 77.593 | 65.122 | 1.00 | 50.21 | 6 |
| ATOM | 18335 | SD | MET | G | 1212 | 62.856 | 75.990 | 64.939 | 1.00 | 51.78 | 16 |
| ATOM | 18336 | CE | MET | G | 1212 | 64.604 | 76.502 | 64.872 | 1.00 | 49.70 | 6 |
| ATOM | 18337 | C | MET | G | 1212 | 61.193 | 79.849 | 68.203 | 1.00 | 62.10 | 6 |
| ATOM | 18338 | O | MET | G | 1212 | 61.004 | 79.070 | 69.141 | 1.00 | 63.42 | 8 |
| ATOM | 18339 | N | ALA | G | 1213 | 61.432 | 81.151 | 68.359 | 1.00 | 42.54 | 7 |
| ATOM | 18340 | CA | ALA | G | 1213 | 61.502 | 81.775 | 69.671 | 1.00 | 42.28 | 6 |
| ATOM | 18341 | CB | ALA | G | 1213 | 60.118 | 81.860 | 70.271 | 1.00 | 13.87 | 6 |
| ATOM | 18342 | C | ALA | G | 1213 | 62.409 | 80.934 | 70.547 | 1.00 | 43.53 | 6 |
| ATOM | 18343 | O | ALA | G | 1213 | 62.038 | 80.502 | 71.633 | 1.00 | 44.76 | 8 |
| ATOM | 18344 | N | ARG | G | 1214 | 63.612 | 80.705 | 70.053 | 1.00 | 30.09 | 7 |
| ATOM | 18345 | CA | ARG | G | 1214 | 64.593 | 79.896 | 70.748 | 1.00 | 31.85 | 6 |
| ATOM | 18346 | CB | ARG | G | 1214 | 64.325 | 78.418 | 70.423 | 1.00 | 71.16 | 6 |
| ATOM | 18347 | CG | ARG | G | 1214 | 65.482 | 77.606 | 69.845 | 1.00 | 75.73 | 6 |
| ATOM | 18348 | CD | ARG | G | 1214 | 65.620 | 76.294 | 70.624 | 1.00 | 79.41 | 6 |
| ATOM | 18349 | NE | ARG | G | 1214 | 66.783 | 75.489 | 70.253 | 1.00 | 79.98 | 7 |
| ATOM | 18350 | CZ | ARG | G | 1214 | 67.300 | 74.523 | 71.014 | 1.00 | 80.05 | 6 |
| ATOM | 18351 | NH1 | ARG | G | 1214 | 66.769 | 74.230 | 72.201 | 1.00 | 79.65 | 7 |
| ATOM | 18352 | NH2 | ARG | G | 1214 | 68.349 | 73.839 | 70.581 | 1.00 | 80.23 | 7 |
| ATOM | 18353 | C | ARG | G | 1214 | 65.923 | 80.379 | 70.193 | 1.00 | 32.25 | 6 |
| ATOM | 18354 | O | ARG | G | 1214 | 65.961 | 81.314 | 69.407 | 1.00 | 31.85 | 8 |
| ATOM | 18355 | N | PRO | G | 1215 | 67.035 | 79.800 | 70.626 | 1.00 | 33.76 | 7 |
| ATOM | 18356 | CD | PRO | G | 1215 | 67.392 | 79.152 | 71.893 | 1.00 | 30.02 | 6 |
| ATOM | 18357 | CA | PRO | G | 1215 | 68.215 | 80.369 | 69.990 | 1.00 | 35.06 | 6 |
| ATOM | 18358 | CB | PRO | G | 1215 | 69.311 | 80.123 | 71.027 | 1.00 | 31.38 | 6 |
| ATOM | 18359 | CG | PRO | G | 1215 | 68.858 | 78.901 | 71.706 | 1.00 | 31.25 | 6 |
| ATOM | 18360 | C | PRO | G | 1215 | 68.529 | 79.760 | 68.612 | 1.00 | 35.27 | 6 |
| ATOM | 18361 | O | PRO | G | 1215 | 68.301 | 78.575 | 68.362 | 1.00 | 36.99 | 8 |
| ATOM | 18362 | N | VAL | G | 1216 | 69.045 | 80.616 | 67.734 | 1.00 | 28.45 | 7 |
| ATOM | 18363 | CA | VAL | G | 1216 | 69.442 | 80.298 | 66.366 | 1.00 | 28.73 | 6 |
| ATOM | 18364 | CB | VAL | G | 1216 | 70.324 | 81.387 | 65.793 | 1.00 | 26.62 | 6 |
| ATOM | 18365 | CG1 | VAL | G | 1216 | 70.832 | 80.978 | 64.427 | 1.00 | 26.35 | 6 |
| ATOM | 18366 | CG2 | VAL | G | 1216 | 69.557 | 82.669 | 65.746 | 1.00 | 26.19 | 6 |
| ATOM | 18367 | C | VAL | G | 1216 | 70.224 | 79.026 | 66.151 | 1.00 | 30.04 | 6 |
| ATOM | 18368 | O | VAL | G | 1216 | 71.364 | 78.902 | 66.601 | 1.00 | 31.31 | 8 |
| ATOM | 18369 | N | SER | G | 1217 | 69.633 | 78.101 | 65.410 | 1.00 | 25.13 | 7 |
| ATOM | 18370 | CA | SER | G | 1217 | 70.310 | 76.859 | 65.118 | 1.00 | 25.66 | 6 |
| ATOM | 18371 | CB | SER | G | 1217 | 69.321 | 75.859 | 64.526 | 1.00 | 77.20 | 6 |
| ATOM | 18372 | OG | SER | G | 1217 | 69.969 | 74.633 | 64.230 | 1.00 | 80.87 | 8 |
| ATOM | 18373 | C | SER | G | 1217 | 71.471 | 77.105 | 64.142 | 1.00 | 25.05 | 6 |
| ATOM | 18374 | O | SER | G | 1217 | 71.430 | 78.025 | 63.318 | 1.00 | 26.30 | 8 |
| ATOM | 18375 | N | ILE | G | 1218 | 72.513 | 76.285 | 64.270 | 1.00 | 19.14 | 7 |
| ATOM | 18376 | CA | ILE | G | 1218 | 73.693 | 76.349 | 63.416 | 1.00 | 18.95 | 6 |
| ATOM | 18377 | CB | ILE | G | 1218 | 74.800 | 75.403 | 63.915 | 1.00 | 52.29 | 6 |
| ATOM | 18378 | CG2 | ILE | G | 1218 | 75.953 | 75.387 | 62.922 | 1.00 | 52.38 | 6 |
| ATOM | 18379 | CG1 | ILE | G | 1218 | 75.299 | 75.846 | 65.285 | 1.00 | 52.07 | 6 |
| ATOM | 18380 | CD1 | ILE | G | 1218 | 76.138 | 77.067 | 65.225 | 1.00 | 51.14 | 6 |
| ATOM | 18381 | C | ILE | G | 1218 | 73.277 | 75.869 | 62.041 | 1.00 | 19.22 | 6 |
| ATOM | 18382 | O | ILE | G | 1218 | 72.591 | 74.858 | 61.919 | 1.00 | 18.58 | 8 |
| ATOM | 18383 | N | GLY | G | 1219 | 73.711 | 76.581 | 61.012 | 1.00 | 39.45 | 7 |
| ATOM | 18384 | CA | GLY | G | 1219 | 73.351 | 76.199 | 59.667 | 1.00 | 39.72 | 6 |
| ATOM | 18385 | C | GLY | G | 1219 | 72.193 | 77.052 | 59.216 | 1.00 | 40.45 | 6 |
| ATOM | 18386 | O | GLY | G | 1219 | 71.640 | 76.851 | 58.138 | 1.00 | 41.07 | 8 |
| ATOM | 18387 | N | GLU | G | 1220 | 71.822 | 78.003 | 60.065 | 1.00 | 30.39 | 7 |
| ATOM | 18388 | CA | GLU | G | 1220 | 70.740 | 78.935 | 59.783 | 1.00 | 30.60 | 6 |
| ATOM | 18389 | CB | GLU | G | 1220 | 70.400 | 79.696 | 61.056 | 1.00 | 68.18 | 6 |
| ATOM | 18390 | CG | GLU | G | 1220 | 69.224 | 80.617 | 60.937 | 1.00 | 72.48 | 6 |
| ATOM | 18391 | CD | GLU | G | 1220 | 67.965 | 79.885 | 60.550 | 1.00 | 73.57 | 6 |
| ATOM | 18392 | OE1 | GLU | G | 1220 | 67.774 | 78.746 | 61.032 | 1.00 | 73.24 | 8 |
| ATOM | 18393 | OE2 | GLU | G | 1220 | 67.159 | 80.454 | 59.780 | 1.00 | 74.87 | 8 |
| ATOM | 18394 | C | GLU | G | 1220 | 71.294 | 79.884 | 58.738 | 1.00 | 29.82 | 6 |
| ATOM | 18395 | O | GLU | G | 1220 | 71.991 | 80.827 | 59.068 | 1.00 | 30.65 | 8 |
| ATOM | 18396 | N | ALA | G | 1221 | 71.008 | 79.628 | 57.473 | 1.00 | 36.15 | 7 |
| ATOM | 18397 | CA | ALA | G | 1221 | 71.538 | 80.476 | 56.413 | 1.00 | 36.74 | 6 |
| ATOM | 18398 | CB | ALA | G | 1221 | 71.163 | 79.885 | 55.054 | 1.00 | 26.70 | 6 |
| ATOM | 18399 | C | ALA | G | 1221 | 71.029 | 81.912 | 56.540 | 1.00 | 37.26 | 6 |
| ATOM | 18400 | O | ALA | G | 1221 | 70.414 | 82.449 | 55.615 | 1.00 | 37.62 | 8 |
| ATOM | 18401 | N | VAL | G | 1222 | 71.306 | 82.540 | 57.681 | 1.00 | 40.17 | 7 |
| ATOM | 18402 | CA | VAL | G | 1222 | 70.839 | 83.903 | 57.943 | 1.00 | 41.30 | 6 |
| ATOM | 18403 | CB | VAL | G | 1222 | 71.536 | 84.526 | 59.184 | 1.00 | 39.52 | 6 |
| ATOM | 18404 | CG1 | VAL | G | 1222 | 71.026 | 85.958 | 59.400 | 1.00 | 40.48 | 6 |
| ATOM | 18405 | CG2 | VAL | G | 1222 | 71.265 | 83.685 | 60.418 | 1.00 | 38.83 | 6 |
| ATOM | 18406 | C | VAL | G | 1222 | 71.014 | 84.856 | 56.767 | 1.00 | 41.79 | 6 |
| ATOM | 18407 | O | VAL | G | 1222 | 70.105 | 85.603 | 56.430 | 1.00 | 42.32 | 8 |
| ATOM | 18408 | N | GLY | G | 1223 | 72.185 | 84.826 | 56.148 | 1.00 | 25.94 | 7 |
| ATOM | 18409 | CA | GLY | G | 1223 | 72.436 | 85.708 | 55.036 | 1.00 | 26.56 | 6 |
| ATOM | 18410 | C | GLY | G | 1223 | 71.166 | 86.043 | 54.291 | 1.00 | 27.30 | 6 |
| ATOM | 18411 | O | GLY | G | 1223 | 70.644 | 87.156 | 54.412 | 1.00 | 28.59 | 8 |
| ATOM | 18412 | N | VAL | G | 1224 | 70.651 | 85.070 | 53.541 | 1.00 | 46.12 | 7 |
| ATOM | 18413 | CA | VAL | G | 1224 | 69.456 | 85.279 | 52.741 | 1.00 | 45.23 | 6 |
| ATOM | 18414 | CB | VAL | G | 1224 | 69.266 | 84.201 | 51.700 | 1.00 | 45.94 | 6 |
| ATOM | 18415 | CG1 | VAL | G | 1224 | 68.407 | 84.743 | 50.590 | 1.00 | 46.95 | 6 |
| ATOM | 18416 | CG2 | VAL | G | 1224 | 70.600 | 83.729 | 51.186 | 1.00 | 45.51 | 6 |
| ATOM | 18417 | C | VAL | G | 1224 | 68.198 | 85.334 | 53.556 | 1.00 | 44.20 | 6 |
| ATOM | 18418 | O | VAL | G | 1224 | 67.167 | 85.771 | 53.062 | 1.00 | 46.29 | 8 |
| ATOM | 18419 | N | VAL | G | 1225 | 68.262 | 84.855 | 54.792 | 1.00 | 20.28 | 7 |
| ATOM | 18420 | CA | VAL | G | 1225 | 67.097 | 84.922 | 55.668 | 1.00 | 17.78 | 6 |
| ATOM | 18421 | CB | VAL | G | 1225 | 67.167 | 83.925 | 56.822 | 1.00 | 27.15 | 6 |
| ATOM | 18422 | CG1 | VAL | G | 1225 | 65.895 | 83.980 | 57.630 | 1.00 | 27.09 | 6 |
| ATOM | 18423 | CG2 | VAL | G | 1225 | 67.399 | 82.536 | 56.282 | 1.00 | 27.80 | 6 |
| ATOM | 18424 | C | VAL | G | 1225 | 67.173 | 86.307 | 56.273 | 1.00 | 17.91 | 6 |
| ATOM | 18425 | O | VAL | G | 1225 | 66.409 | 86.640 | 57.179 | 1.00 | 16.87 | 8 |
| ATOM | 18426 | N | ALA | G | 1226 | 68.121 | 87.099 | 55.762 | 1.00 | 34.77 | 7 |
| ATOM | 18427 | CA | ALA | G | 1226 | 68.361 | 88.458 | 56.227 | 1.00 | 35.41 | 6 |
| ATOM | 18428 | CB | ALA | G | 1226 | 69.771 | 88.582 | 56.770 | 1.00 | 35.47 | 6 |
| ATOM | 18429 | C | ALA | G | 1226 | 68.146 | 89.450 | 55.107 | 1.00 | 35.70 | 6 |
| ATOM | 18430 | O | ALA | G | 1226 | 67.928 | 90.628 | 55.351 | 1.00 | 36.77 | 8 |
| ATOM | 18431 | N | ALA | G | 1227 | 68.228 | 88.982 | 53.872 | 1.00 | 40.98 | 7 |
| ATOM | 18432 | CA | ALA | G | 1227 | 67.988 | 89.865 | 52.740 | 1.00 | 42.72 | 6 |
| ATOM | 18433 | CB | ALA | G | 1227 | 68.731 | 89.369 | 51.513 | 1.00 | 60.17 | 6 |
| ATOM | 18434 | C | ALA | G | 1227 | 66.474 | 89.844 | 52.501 | 1.00 | 43.37 | 6 |
| ATOM | 18435 | O | ALA | G | 1227 | 65.833 | 90.890 | 52.433 | 1.00 | 44.26 | 8 |
| ATOM | 18436 | N | GLU | G | 1228 | 65.905 | 88.646 | 52.397 | 1.00 | 26.42 | 7 |
| ATOM | 18437 | CA | GLU | G | 1228 | 64.484 | 88.498 | 52.189 | 1.00 | 26.29 | 6 |
| ATOM | 18438 | CB | GLU | G | 1228 | 64.089 | 87.041 | 52.355 | 1.00 | 30.18 | 6 |
| ATOM | 18439 | CG | GLU | G | 1228 | 64.838 | 86.122 | 51.410 | 1.00 | 35.78 | 6 |
| ATOM | 18440 | CD | GLU | G | 1228 | 64.256 | 84.727 | 51.338 | 1.00 | 39.43 | 6 |
| ATOM | 18441 | OE1 | GLU | G | 1228 | 64.268 | 84.026 | 52.373 | 1.00 | 41.42 | 8 |
| ATOM | 18442 | OE2 | GLU | G | 1228 | 63.786 | 84.335 | 50.243 | 1.00 | 43.56 | 8 |
| ATOM | 18443 | C | GLU | G | 1228 | 63.767 | 89.352 | 53.206 | 1.00 | 26.75 | 6 |
| ATOM | 18444 | O | GLU | G | 1228 | 63.012 | 90.238 | 52.843 | 1.00 | 27.89 | 8 |
| ATOM | 18445 | N | SER | G | 1229 | 64.018 | 89.100 | 54.486 | 1.00 | 28.76 | 7 |
| ATOM | 18446 | CA | SER | G | 1229 | 63.379 | 89.860 | 55.558 | 1.00 | 30.26 | 6 |
| ATOM | 18447 | CB | SER | G | 1229 | 64.133 | 89.685 | 56.873 | 1.00 | 58.30 | 6 |
| ATOM | 18448 | OG | SER | G | 1229 | 63.868 | 88.427 | 57.453 | 1.00 | 59.97 | 8 |
| ATOM | 18449 | C | SER | G | 1229 | 63.326 | 91.332 | 55.233 | 1.00 | 31.99 | 6 |
| ATOM | 18450 | O | SER | G | 1229 | 62.278 | 91.953 | 55.284 | 1.00 | 33.06 | 8 |
| ATOM | 18451 | N | ILE | G | 1230 | 64.471 | 91.894 | 54.899 | 1.00 | 22.94 | 7 |
| ATOM | 18452 | CA | ILE | G | 1230 | 64.537 | 93.301 | 54.584 | 1.00 | 26.92 | 6 |
| ATOM | 18453 | CB | ILE | G | 1230 | 65.997 | 93.785 | 54.575 | 1.00 | 52.11 | 6 |
| ATOM | 18454 | CG2 | ILE | G | 1230 | 66.037 | 95.296 | 54.369 | 1.00 | 52.92 | 6 |
| ATOM | 18455 | CG1 | ILE | G | 1230 | 66.694 | 93.370 | 55.877 | 1.00 | 52.92 | 6 |
| ATOM | 18456 | CD1 | ILE | G | 1230 | 68.165 | 93.726 | 55.938 | 1.00 | 53.75 | 6 |
| ATOM | 18457 | C | ILE | G | 1230 | 63.922 | 93.617 | 53.229 | 1.00 | 28.48 | 6 |
| ATOM | 18458 | O | ILE | G | 1230 | 63.670 | 94.766 | 52.928 | 1.00 | 30.31 | 8 |
| ATOM | 18459 | N | GLY | G | 1231 | 63.664 | 92.610 | 52.411 | 1.00 | 37.73 | 7 |
| ATOM | 18460 | CA | GLY | G | 1231 | 63.128 | 92.884 | 51.089 | 1.00 | 40.38 | 6 |
| ATOM | 18461 | C | GLY | G | 1231 | 61.656 | 92.643 | 50.830 | 1.00 | 42.90 | 6 |
| ATOM | 18462 | O | GLY | G | 1231 | 61.122 | 93.149 | 49.847 | 1.00 | 43.23 | 8 |
| ATOM | 18463 | N | GLU | G | 1232 | 61.003 | 91.847 | 51.668 | 1.00 | 47.02 | 7 |
| ATOM | 18464 | CA | GLU | G | 1232 | 59.579 | 91.609 | 51.491 | 1.00 | 51.50 | 6 |
| ATOM | 18465 | CB | GLU | G | 1232 | 59.086 | 90.475 | 52.396 | 1.00 | 199.28 | 6 |
| ATOM | 18466 | CG | GLU | G | 1232 | 57.595 | 90.190 | 52.263 | 1.00 | 204.27 | 6 |
| ATOM | 18467 | CD | GLU | G | 1232 | 56.882 | 90.144 | 53.602 | 1.00 | 207.53 | 6 |
| ATOM | 18468 | OE1 | GLU | G | 1232 | 57.299 | 89.355 | 54.479 | 1.00 | 208.87 | 8 |
| ATOM | 18469 | OE2 | GLU | G | 1232 | 55.898 | 90.895 | 53.776 | 1.00 | 208.87 | 8 |
| ATOM | 18470 | C | GLU | G | 1232 | 58.901 | 92.926 | 51.873 | 1.00 | 52.75 | 6 |
| ATOM | 18471 | O | GLU | G | 1232 | 58.267 | 93.576 | 51.034 | 1.00 | 53.88 | 8 |
| ATOM | 18472 | N | PRO | G | 1233 | 59.039 | 93.354 | 53.141 | 1.00 | 55.76 | 7 |
| ATOM | 18473 | CD | PRO | G | 1233 | 59.754 | 92.757 | 54.284 | 1.00 | 99.43 | 6 |
| ATOM | 18474 | CA | PRO | G | 1233 | 58.407 | 94.614 | 53.533 | 1.00 | 56.78 | 6 |
| ATOM | 18475 | CB | PRO | G | 1233 | 58.981 | 94.851 | 54.919 | 1.00 | 100.11 | 6 |
| ATOM | 18476 | CG | PRO | G | 1233 | 59.121 | 93.456 | 55.453 | 1.00 | 99.64 | 6 |
| ATOM | 18477 | C | PRO | G | 1233 | 58.776 | 95.723 | 52.543 | 1.00 | 56.76 | 6 |
| ATOM | 18478 | O | PRO | G | 1233 | 58.176 | 96.792 | 52.527 | 1.00 | 57.07 | 8 |
| ATOM | 18479 | N | GLY | G | 1234 | 59.775 | 95.452 | 51.718 | 1.00 | 47.53 | 7 |
| ATOM | 18480 | CA | GLY | G | 1234 | 60.191 | 96.423 | 50.734 | 1.00 | 50.03 | 6 |
| ATOM | 18481 | C | GLY | G | 1234 | 59.154 | 96.573 | 49.641 | 1.00 | 51.01 | 6 |
| ATOM | 18482 | O | GLY | G | 1234 | 59.370 | 97.283 | 48.657 | 1.00 | 51.04 | 8 |
| ATOM | 18483 | N | THR | G | 1235 | 58.023 | 95.900 | 49.798 | 1.00 | 102.30 | 7 |
| ATOM | 18484 | CA | THR | G | 1235 | 56.971 | 95.994 | 48.803 | 1.00 | 104.55 | 6 |
| ATOM | 18485 | CB | THR | G | 1235 | 56.427 | 94.601 | 48.455 | 1.00 | 95.64 | 6 |
| ATOM | 18486 | OG1 | THR | G | 1235 | 57.521 | 93.750 | 48.089 | 1.00 | 96.80 | 8 |
| ATOM | 18487 | CG2 | THR | G | 1235 | 55.460 | 94.682 | 47.284 | 1.00 | 96.40 | 6 |
| ATOM | 18488 | C | THR | G | 1235 | 55.874 | 96.880 | 49.377 | 1.00 | 104.60 | 6 |
| ATOM | 18489 | O | THR | G | 1235 | 54.697 | 96.746 | 49.049 | 1.00 | 105.19 | 8 |
| ATOM | 18490 | N | GLN | G | 1236 | 56.291 | 97.795 | 50.243 | 1.00 | 131.07 | 7 |
| ATOM | 18491 | CA | GLN | G | 1236 | 55.382 | 98.727 | 50.884 | 1.00 | 131.35 | 6 |
| ATOM | 18492 | CB | GLN | G | 1236 | 54.976 | 98.221 | 52.262 | 1.00 | 101.39 | 6 |
| ATOM | 18493 | CG | GLN | G | 1236 | 54.507 | 96.791 | 52.313 | 1.00 | 100.77 | 6 |
| ATOM | 18494 | CD | GLN | G | 1236 | 54.127 | 96.403 | 53.716 | 1.00 | 100.20 | 6 |
| ATOM | 18495 | OE1 | GLN | G | 1236 | 54.941 | 96.481 | 54.634 | 1.00 | 98.99 | 8 |
| ATOM | 18496 | NE2 | GLN | G | 1236 | 52.880 | 95.995 | 53.899 | 1.00 | 101.11 | 7 |
| ATOM | 18497 | C | GLN | G | 1236 | 56.103 | 100.050 | 51.050 | 1.00 | 131.57 | 6 |
| ATOM | 18498 | O | GLN | G | 1236 | 56.101 | 100.631 | 52.135 | 1.00 | 131.80 | 8 |
| ATOM | 18499 | N | LEU | G | 1237 | 56.730 | 100.521 | 49.979 | 1.00 | 68.64 | 7 |
| ATOM | 18500 | CA | LEU | G | 1237 | 57.460 | 101.782 | 50.028 | 1.00 | 68.64 | 6 |
| ATOM | 18501 | CB | LEU | G | 1237 | 58.947 | 101.514 | 50.216 | 1.00 | 28.36 | 6 |
| ATOM | 18502 | CG | LEU | G | 1237 | 59.410 | 100.516 | 51.267 | 1.00 | 27.11 | 6 |
| ATOM | 18503 | CD1 | LEU | G | 1237 | 60.925 | 100.605 | 51.322 | 1.00 | 26.70 | 6 |
| ATOM | 18504 | CD2 | LEU | G | 1237 | 58.805 | 100.801 | 52.635 | 1.00 | 25.91 | 6 |
| ATOM | 18505 | C | LEU | G | 1237 | 57.284 | 102.625 | 48.772 | 1.00 | 70.01 | 6 |
| ATOM | 18506 | O | LEU | G | 1237 | 57.446 | 102.132 | 47.656 | 1.00 | 70.65 | 8 |
| ATOM | 18507 | N | THR | G | 1238 | 56.968 | 103.901 | 48.956 | 1.00 | 95.54 | 7 |
| ATOM | 18508 | CA | THR | G | 1238 | 56.800 | 104.809 | 47.827 | 1.00 | 97.27 | 6 |
| ATOM | 18509 | CB | THR | G | 1238 | 56.745 | 106.267 | 48.306 | 1.00 | 120.99 | 6 |
| ATOM | 18510 | OG1 | THR | G | 1238 | 55.718 | 106.402 | 49.298 | 1.00 | 120.97 | 8 |
| ATOM | 18511 | CG2 | THR | G | 1238 | 56.456 | 107.201 | 47.137 | 1.00 | 121.01 | 6 |
| ATOM | 18512 | C | THR | G | 1238 | 57.988 | 104.634 | 46.876 | 1.00 | 98.11 | 6 |
| ATOM | 18513 | O | THR | G | 1238 | 59.052 | 104.171 | 47.289 | 1.00 | 98.67 | 8 |
| ATOM | 18514 | N | MET | G | 1239 | 57.815 | 105.002 | 45.611 | 1.00 | 90.25 | 7 |
| ATOM | 18515 | CA | MET | G | 1239 | 58.894 | 104.838 | 44.644 | 1.00 | 91.46 | 6 |
| ATOM | 18516 | CB | MET | G | 1239 | 58.542 | 103.713 | 43.669 | 1.00 | 80.46 | 6 |
| ATOM | 18517 | CG | MET | G | 1239 | 59.572 | 103.454 | 42.572 | 1.00 | 80.17 | 6 |
| ATOM | 18518 | SD | MET | G | 1239 | 61.150 | 102.780 | 43.144 | 1.00 | 79.14 | 16 |
| ATOM | 18519 | CE | MET | G | 1239 | 62.270 | 104.163 | 42.830 | 1.00 | 79.80 | 6 |
| ATOM | 18520 | C | MET | G | 1239 | 59.209 | 106.105 | 43.867 | 1.00 | 92.59 | 6 |
| ATOM | 18521 | O | MET | G | 1239 | 60.097 | 106.112 | 43.010 | 1.00 | 92.57 | 8 |
| ATOM | 18522 | N | ALA | G | 1240 | 58.492 | 107.181 | 44.172 | 1.00 | 165.07 | 7 |
| ATOM | 18523 | CA | ALA | G | 1240 | 58.703 | 108.443 | 43.476 | 1.00 | 166.48 | 6 |
| ATOM | 18524 | CB | ALA | G | 1240 | 57.382 | 108.952 | 42.921 | 1.00 | 169.27 | 6 |
| ATOM | 18525 | C | ALA | G | 1240 | 59.344 | 109.522 | 44.335 | 1.00 | 166.70 | 6 |
| ATOM | 18526 | O | ALA | G | 1240 | 59.209 | 109.528 | 45.559 | 1.00 | 166.35 | 8 |
| ATOM | 18527 | N | ALA | G | 1241 | 60.045 | 110.437 | 43.677 | 1.00 | 131.55 | 7 |
| ATOM | 18528 | CA | ALA | G | 1241 | 60.687 | 111.539 | 44.370 | 1.00 | 132.39 | 6 |
| ATOM | 18529 | CB | ALA | G | 1241 | 62.043 | 111.836 | 43.743 | 1.00 | 177.09 | 6 |
| ATOM | 18530 | C | ALA | G | 1241 | 59.760 | 112.741 | 44.232 | 1.00 | 133.05 | 6 |
| ATOM | 18531 | O | ALA | G | 1241 | 58.839 | 112.671 | 43.385 | 1.00 | 133.52 | 8 |
| ATOM | 18532 | OXT | ALA | G | 1241 | 59.963 | 113.732 | 44.966 | 1.00 | 178.77 | 8 |
| ATOM | 18533 | C | GLY | M | 1250 | 63.645 | 118.570 | 53.899 | 1.00 | 135.19 | 6 |
| ATOM | 18534 | O | GLY | M | 1250 | 64.510 | 119.354 | 53.505 | 1.00 | 135.32 | 8 |
| ATOM | 18535 | N | GLY | M | 1250 | 61.507 | 119.633 | 54.623 | 1.00 | 135.29 | 7 |
| ATOM | 18536 | CA | GLY | M | 1250 | 62.745 | 118.934 | 55.062 | 1.00 | 135.34 | 6 |
| ATOM | 18537 | N | THR | M | 1251 | 63.438 | 117.379 | 53.347 | 1.00 | 165.37 | 7 |
| ATOM | 18538 | CA | THR | M | 1251 | 64.232 | 116.894 | 52.223 | 1.00 | 164.97 | 6 |
| ATOM | 18539 | CB | THR | M | 1251 | 65.724 | 116.718 | 52.622 | 1.00 | 135.22 | 6 |
| ATOM | 18540 | OG1 | THR | M | 1251 | 66.439 | 117.928 | 52.342 | 1.00 | 136.14 | 8 |
| ATOM | 18541 | CG2 | THR | M | 1251 | 66.370 | 115.564 | 51.862 | 1.00 | 134.02 | 6 |
| ATOM | 18542 | C | THR | M | 1251 | 63.703 | 115.564 | 51.710 | 1.00 | 164.45 | 6 |
| ATOM | 18543 | O | THR | M | 1251 | 63.640 | 114.588 | 52.457 | 1.00 | 165.00 | 8 |
| ATOM | 18544 | N | ASP | M | 1252 | 63.320 | 115.534 | 50.434 | 1.00 | 140.34 | 7 |
| ATOM | 18545 | CA | ASP | M | 1252 | 62.817 | 114.310 | 49.816 | 1.00 | 138.24 | 6 |
| ATOM | 18546 | CB | ASP | M | 1252 | 62.416 | 114.544 | 48.352 | 1.00 | 81.45 | 6 |
| ATOM | 18547 | CG | ASP | M | 1252 | 61.132 | 115.342 | 48.206 | 1.00 | 79.98 | 6 |
| ATOM | 18548 | OD1 | ASP | M | 1252 | 60.111 | 114.970 | 48.821 | 1.00 | 78.97 | 8 |
| ATOM | 18549 | OD2 | ASP | M | 1252 | 61.143 | 116.337 | 47.454 | 1.00 | 78.82 | 8 |
| ATOM | 18550 | C | ASP | M | 1252 | 63.915 | 113.255 | 49.861 | 1.00 | 137.69 | 6 |
| ATOM | 18551 | O | ASP | M | 1252 | 64.943 | 113.447 | 50.513 | 1.00 | 138.06 | 8 |
| ATOM | 18552 | N | ILE | M | 1253 | 63.698 | 112.149 | 49.153 | 1.00 | 78.27 | 7 |
| ATOM | 18553 | CA | ILE | M | 1253 | 64.657 | 111.051 | 49.116 | 1.00 | 76.99 | 6 |
| ATOM | 18554 | CB | ILE | M | 1253 | 66.058 | 111.538 | 48.705 | 1.00 | 150.94 | 6 |
| ATOM | 18555 | CG2 | ILE | M | 1253 | 67.068 | 110.455 | 48.972 | 1.00 | 150.19 | 6 |
| ATOM | 18556 | CG1 | ILE | M | 1253 | 66.060 | 111.943 | 47.227 | 1.00 | 151.66 | 6 |
| ATOM | 18557 | CD1 | ILE | M | 1253 | 67.401 | 112.440 | 46.716 | 1.00 | 152.48 | 6 |
| ATOM | 18558 | C | ILE | M | 1253 | 64.723 | 110.448 | 50.506 | 1.00 | 75.67 | 6 |
| ATOM | 18559 | O | ILE | M | 1253 | 64.930 | 109.247 | 50.677 | 1.00 | 75.98 | 8 |
| ATOM | 18560 | N | THR | M | 1254 | 64.548 | 111.311 | 51.497 | 1.00 | 94.13 | 7 |
| ATOM | 18561 | CA | THR | M | 1254 | 64.542 | 110.910 | 52.890 | 1.00 | 92.70 | 6 |
| ATOM | 18562 | CB | THR | M | 1254 | 64.702 | 112.154 | 53.831 | 1.00 | 85.16 | 6 |
| ATOM | 18563 | OG1 | THR | M | 1254 | 65.892 | 112.880 | 53.489 | 1.00 | 83.56 | 8 |
| ATOM | 18564 | CG2 | THR | M | 1254 | 64.806 | 111.721 | 55.279 | 1.00 | 85.77 | 6 |
| ATOM | 18565 | C | THR | M | 1254 | 63.160 | 110.266 | 53.063 | 1.00 | 91.19 | 6 |
| ATOM | 18566 | O | THR | M | 1254 | 62.555 | 110.320 | 54.133 | 1.00 | 91.52 | 8 |
| ATOM | 18567 | N | GLN | M | 1255 | 62.675 | 109.656 | 51.982 | 1.00 | 65.57 | 7 |
| ATOM | 18568 | CA | GLN | M | 1255 | 61.372 | 109.011 | 51.969 | 1.00 | 62.64 | 6 |
| ATOM | 18569 | CB | GLN | M | 1255 | 60.315 | 109.979 | 51.442 | 1.00 | 146.40 | 6 |
| ATOM | 18570 | CG | GLN | M | 1255 | 60.056 | 111.181 | 52.319 | 1.00 | 148.22 | 6 |
| ATOM | 18571 | CD | GLN | M | 1255 | 59.021 | 112.101 | 51.712 | 1.00 | 149.95 | 6 |
| ATOM | 18572 | OE1 | GLN | M | 1255 | 57.926 | 111.668 | 51.360 | 1.00 | 150.11 | 8 |
| ATOM | 18573 | NE2 | GLN | M | 1255 | 59.361 | 113.377 | 51.587 | 1.00 | 152.05 | 7 |
| ATOM | 18574 | C | GLN | M | 1255 | 61.304 | 107.737 | 51.129 | 1.00 | 59.98 | 6 |
| ATOM | 18575 | O | GLN | M | 1255 | 61.989 | 107.595 | 50.113 | 1.00 | 60.25 | 8 |
| ATOM | 18576 | N | GLY | M | 1256 | 60.453 | 106.818 | 51.578 | 1.00 | 87.11 | 7 |
| ATOM | 18577 | CA | GLY | M | 1256 | 60.222 | 105.561 | 50.883 | 1.00 | 81.72 | 6 |
| ATOM | 18578 | C | GLY | M | 1256 | 61.389 | 104.677 | 50.505 | 1.00 | 77.98 | 6 |
| ATOM | 18579 | O | GLY | M | 1256 | 62.517 | 104.871 | 50.948 | 1.00 | 77.97 | 8 |
| ATOM | 18580 | N | LEU | M | 1257 | 61.086 | 103.693 | 49.667 | 1.00 | 63.28 | 7 |
| ATOM | 18581 | CA | LEU | M | 1257 | 62.063 | 102.727 | 49.195 | 1.00 | 59.66 | 6 |
| ATOM | 18582 | CB | LEU | M | 1257 | 61.394 | 101.738 | 48.230 | 1.00 | 66.92 | 6 |
| ATOM | 18583 | CG | LEU | M | 1257 | 62.275 | 100.661 | 47.595 | 1.00 | 67.71 | 6 |
| ATOM | 18584 | CD1 | LEU | M | 1257 | 63.040 | 99.906 | 48.667 | 1.00 | 69.39 | 6 |
| ATOM | 18585 | CD2 | LEU | M | 1257 | 61.412 | 99.716 | 46.793 | 1.00 | 68.69 | 6 |
| ATOM | 18586 | C | LEU | M | 1257 | 63.282 | 103.357 | 48.528 | 1.00 | 56.45 | 6 |
| ATOM | 18587 | O | LEU | M | 1257 | 64.404 | 102.878 | 48.697 | 1.00 | 57.55 | 8 |
| ATOM | 18588 | N | PRO | M | 1258 | 63.089 | 104.439 | 47.762 | 1.00 | 19.94 | 7 |
| ATOM | 18589 | CD | PRO | M | 1258 | 61.880 | 105.163 | 47.348 | 1.00 | 15.83 | 6 |
| ATOM | 18590 | CA | PRO | M | 1258 | 64.274 | 105.018 | 47.135 | 1.00 | 17.31 | 6 |
| ATOM | 18591 | CB | PRO | M | 1258 | 63.720 | 106.248 | 46.448 | 1.00 | 14.49 | 6 |
| ATOM | 18592 | CG | PRO | M | 1258 | 62.347 | 105.820 | 46.081 | 1.00 | 14.23 | 6 |
| ATOM | 18593 | C | PRO | M | 1258 | 65.343 | 105.342 | 48.174 | 1.00 | 15.47 | 6 |
| ATOM | 18594 | O | PRO | M | 1258 | 66.536 | 105.334 | 47.871 | 1.00 | 14.86 | 8 |
| ATOM | 18595 | N | ARG | M | 1259 | 64.915 | 105.626 | 49.402 | 1.00 | 38.68 | 7 |
| ATOM | 18596 | CA | ARG | M | 1259 | 65.865 | 105.922 | 50.462 | 1.00 | 38.22 | 6 |
| ATOM | 18597 | CB | ARG | M | 1259 | 65.207 | 106.649 | 51.628 | 1.00 | 57.23 | 6 |
| ATOM | 18598 | CG | ARG | M | 1259 | 66.203 | 106.853 | 52.745 | 1.00 | 58.34 | 6 |
| ATOM | 18599 | CD | ARG | M | 1259 | 65.911 | 108.044 | 53.623 | 1.00 | 57.68 | 6 |
| ATOM | 18600 | NE | ARG | M | 1259 | 67.073 | 108.386 | 54.445 | 1.00 | 58.11 | 7 |
| ATOM | 18601 | CZ | ARG | M | 1259 | 68.235 | 108.806 | 53.952 | 1.00 | 58.58 | 6 |
| ATOM | 18602 | NH1 | ARG | M | 1259 | 68.405 | 108.940 | 52.643 | 1.00 | 58.25 | 7 |
| ATOM | 18603 | NH2 | ARG | M | 1259 | 69.233 | 109.090 | 54.768 | 1.00 | 58.51 | 7 |
| ATOM | 18604 | C | ARG | M | 1259 | 66.475 | 104.628 | 50.968 | 1.00 | 37.61 | 6 |
| ATOM | 18605 | O | ARG | M | 1259 | 67.696 | 104.481 | 51.009 | 1.00 | 37.32 | 8 |
| ATOM | 18606 | N | VAL | M | 1260 | 65.620 | 103.689 | 51.353 | 1.00 | 32.94 | 7 |
| ATOM | 18607 | CA | VAL | M | 1260 | 66.092 | 102.404 | 51.831 | 1.00 | 32.40 | 6 |
| ATOM | 18608 | CB | VAL | M | 1260 | 64.979 | 101.356 | 51.885 | 1.00 | 23.47 | 6 |
| ATOM | 18609 | CG1 | VAL | M | 1260 | 65.369 | 100.298 | 52.871 | 1.00 | 23.67 | 6 |
| ATOM | 18610 | CG2 | VAL | M | 1260 | 63.652 | 101.979 | 52.240 | 1.00 | 21.57 | 6 |
| ATOM | 18611 | C | VAL | M | 1260 | 67.158 | 101.884 | 50.868 | 1.00 | 33.31 | 6 |
| ATOM | 18612 | O | VAL | M | 1260 | 68.000 | 101.080 | 51.246 | 1.00 | 34.63 | 8 |
| ATOM | 18613 | N | ILE | M | 1261 | 67.101 | 102.319 | 49.614 | 1.00 | 42.94 | 7 |
| ATOM | 18614 | CA | ILE | M | 1261 | 68.094 | 101.914 | 48.627 | 1.00 | 43.12 | 6 |
| ATOM | 18615 | CB | ILE | M | 1261 | 67.543 | 102.059 | 47.170 | 1.00 | 27.14 | 6 |
| ATOM | 18616 | CG2 | ILE | M | 1261 | 68.630 | 102.609 | 46.208 | 1.00 | 25.72 | 6 |
| ATOM | 18617 | CG1 | ILE | M | 1261 | 67.006 | 100.711 | 46.689 | 1.00 | 28.42 | 6 |
| ATOM | 18618 | CD1 | ILE | M | 1261 | 68.076 | 99.636 | 46.488 | 1.00 | 31.25 | 6 |
| ATOM | 18619 | C | ILE | M | 1261 | 69.302 | 102.831 | 48.813 | 1.00 | 43.58 | 6 |
| ATOM | 18620 | O | ILE | M | 1261 | 70.411 | 102.368 | 49.095 | 1.00 | 43.96 | 8 |
| ATOM | 18621 | N | GLU | M | 1262 | 69.064 | 104.133 | 48.669 | 1.00 | 36.27 | 7 |
| ATOM | 18622 | CA | GLU | M | 1262 | 70.100 | 105.146 | 48.796 | 1.00 | 34.97 | 6 |
| ATOM | 18623 | CB | GLU | M | 1262 | 69.445 | 106.524 | 48.929 | 1.00 | 26.80 | 6 |
| ATOM | 18624 | CG | GLU | M | 1262 | 70.393 | 107.671 | 49.294 | 1.00 | 24.59 | 6 |
| ATOM | 18625 | CD | GLU | M | 1262 | 69.711 | 109.048 | 49.269 | 1.00 | 23.28 | 6 |
| ATOM | 18626 | OE1 | GLU | M | 1262 | 69.557 | 109.607 | 48.165 | 1.00 | 20.64 | 8 |
| ATOM | 18627 | OE2 | GLU | M | 1262 | 69.328 | 109.573 | 50.345 | 1.00 | 20.75 | 8 |
| ATOM | 18628 | C | GLU | M | 1262 | 71.044 | 104.880 | 49.961 | 1.00 | 35.19 | 6 |
| ATOM | 18629 | O | GLU | M | 1262 | 72.245 | 105.158 | 49.862 | 1.00 | 34.77 | 8 |
| ATOM | 18630 | N | LEU | M | 1263 | 70.501 | 104.334 | 51.053 | 1.00 | 34.38 | 7 |
| ATOM | 18631 | CA | LEU | M | 1263 | 71.300 | 104.027 | 52.250 | 1.00 | 34.87 | 6 |
| ATOM | 18632 | CB | LEU | M | 1263 | 70.418 | 103.894 | 53.503 | 1.00 | 32.86 | 6 |
| ATOM | 18633 | CG | LEU | M | 1263 | 70.050 | 105.191 | 54.235 | 1.00 | 32.29 | 6 |
| ATOM | 18634 | CD1 | LEU | M | 1263 | 69.261 | 106.073 | 53.304 | 1.00 | 32.18 | 6 |
| ATOM | 18635 | CD2 | LEU | M | 1263 | 69.249 | 104.894 | 55.490 | 1.00 | 32.41 | 6 |
| ATOM | 18636 | C | LEU | M | 1263 | 72.098 | 102.755 | 52.071 | 1.00 | 35.00 | 6 |
| ATOM | 18637 | O | LEU | M | 1263 | 73.320 | 102.773 | 52.141 | 1.00 | 35.89 | 8 |
| ATOM | 18638 | N | PHE | M | 1264 | 71.408 | 101.647 | 51.855 | 1.00 | 36.63 | 7 |
| ATOM | 18639 | CA | PHE | M | 1264 | 72.086 | 100.387 | 51.639 | 1.00 | 38.06 | 6 |
| ATOM | 18640 | CB | PHE | M | 1264 | 71.101 | 99.370 | 51.078 | 1.00 | 33.57 | 6 |
| ATOM | 18641 | CG | PHE | M | 1264 | 70.324 | 98.647 | 52.123 | 1.00 | 31.87 | 6 |
| ATOM | 18642 | CD1 | PHE | M | 1264 | 69.067 | 98.145 | 51.839 | 1.00 | 31.56 | 6 |
| ATOM | 18643 | CD2 | PHE | M | 1264 | 70.865 | 98.429 | 53.379 | 1.00 | 31.81 | 6 |
| ATOM | 18644 | CE1 | PHE | M | 1264 | 68.356 | 97.437 | 52.783 | 1.00 | 31.35 | 6 |
| ATOM | 18645 | CE2 | PHE | M | 1264 | 70.167 | 97.721 | 54.336 | 1.00 | 31.51 | 6 |
| ATOM | 18646 | CZ | PHE | M | 1264 | 68.910 | 97.223 | 54.039 | 1.00 | 31.65 | 6 |
| ATOM | 18647 | C | PHE | M | 1264 | 73.218 | 100.610 | 50.643 | 1.00 | 41.11 | 6 |
| ATOM | 18648 | O | PHE | M | 1264 | 74.280 | 99.991 | 50.736 | 1.00 | 42.16 | 8 |
| ATOM | 18649 | N | GLU | M | 1265 | 72.982 | 101.526 | 49.708 | 1.00 | 56.57 | 7 |
| ATOM | 18650 | CA | GLU | M | 1265 | 73.947 | 101.834 | 48.667 | 1.00 | 58.00 | 6 |
| ATOM | 18651 | CB | GLU | M | 1265 | 73.203 | 102.235 | 47.400 | 1.00 | 60.16 | 6 |
| ATOM | 18652 | CG | GLU | M | 1265 | 73.947 | 101.861 | 46.157 | 1.00 | 61.97 | 6 |
| ATOM | 18653 | CD | GLU | M | 1265 | 74.040 | 100.370 | 46.003 | 1.00 | 63.74 | 6 |
| ATOM | 18654 | OE1 | GLU | M | 1265 | 73.115 | 99.789 | 45.394 | 1.00 | 64.22 | 8 |
| ATOM | 18655 | OE2 | GLU | M | 1265 | 75.026 | 99.786 | 46.507 | 1.00 | 64.82 | 8 |
| ATOM | 18656 | C | GLU | M | 1265 | 74.975 | 102.911 | 49.021 | 1.00 | 58.20 | 6 |
| ATOM | 18657 | O | GLU | M | 1265 | 76.126 | 102.828 | 48.601 | 1.00 | 59.28 | 8 |
| ATOM | 18658 | N | ALA | M | 1266 | 74.563 | 103.928 | 49.770 | 1.00 | 34.07 | 7 |
| ATOM | 18659 | CA | ALA | M | 1266 | 75.477 | 104.997 | 50.174 | 1.00 | 34.49 | 6 |
| ATOM | 18660 | CB | ALA | M | 1266 | 76.760 | 104.403 | 50.737 | 1.00 | 15.71 | 6 |
| ATOM | 18661 | C | ALA | M | 1266 | 75.814 | 105.935 | 49.031 | 1.00 | 34.66 | 6 |
| ATOM | 18662 | O | ALA | M | 1266 | 76.415 | 105.517 | 48.046 | 1.00 | 33.92 | 8 |
| ATOM | 18663 | N | ARG | M | 1267 | 75.436 | 107.205 | 49.183 | 1.00 | 20.97 | 7 |
| ATOM | 18664 | CA | ARG | M | 1267 | 75.675 | 108.251 | 48.176 | 1.00 | 22.91 | 6 |
| ATOM | 18665 | CB | ARG | M | 1267 | 74.741 | 108.084 | 46.977 | 1.00 | 82.15 | 6 |
| ATOM | 18666 | CG | ARG | M | 1267 | 75.058 | 106.924 | 46.081 | 1.00 | 85.87 | 6 |
| ATOM | 18667 | CD | ARG | M | 1267 | 73.828 | 106.517 | 45.311 | 1.00 | 88.20 | 6 |
| ATOM | 18668 | NE | ARG | M | 1267 | 74.045 | 105.280 | 44.577 | 1.00 | 90.48 | 7 |
| ATOM | 18669 | CZ | ARG | M | 1267 | 73.069 | 104.471 | 44.189 | 1.00 | 91.44 | 6 |
| ATOM | 18670 | NH1 | ARG | M | 1267 | 71.808 | 104.772 | 44.471 | 1.00 | 92.06 | 7 |
| ATOM | 18671 | NH2 | ARG | M | 1267 | 73.357 | 103.361 | 43.525 | 1.00 | 92.25 | 7 |
| ATOM | 18672 | C | ARG | M | 1267 | 75.455 | 109.644 | 48.735 | 1.00 | 22.51 | 6 |
| ATOM | 18673 | O | ARG | M | 1267 | 75.015 | 109.811 | 49.855 | 1.00 | 20.90 | 8 |
| ATOM | 18674 | N | ARG | M | 1268 | 75.752 | 110.634 | 47.914 | 1.00 | 37.10 | 7 |
| ATOM | 18675 | CA | ARG | M | 1268 | 75.599 | 112.033 | 48.271 | 1.00 | 40.41 | 6 |
| ATOM | 18676 | CB | ARG | M | 1268 | 76.660 | 112.843 | 47.515 | 1.00 | 77.67 | 6 |
| ATOM | 18677 | CG | ARG | M | 1268 | 76.702 | 114.329 | 47.824 | 1.00 | 79.69 | 6 |
| ATOM | 18678 | CD | ARG | M | 1268 | 77.613 | 114.667 | 49.010 | 1.00 | 82.36 | 6 |
| ATOM | 18679 | NE | ARG | M | 1268 | 77.808 | 116.115 | 49.132 | 1.00 | 85.06 | 7 |
| ATOM | 18680 | CZ | ARG | M | 1268 | 78.622 | 116.701 | 50.005 | 1.00 | 85.75 | 6 |
| ATOM | 18681 | NH1 | ARG | M | 1268 | 79.332 | 115.970 | 50.852 | 1.00 | 87.35 | 7 |
| ATOM | 18682 | NH2 | ARG | M | 1268 | 78.733 | 118.022 | 50.021 | 1.00 | 84.62 | 7 |
| ATOM | 18683 | C | ARG | M | 1268 | 74.174 | 112.480 | 47.878 | 1.00 | 41.76 | 6 |
| ATOM | 18684 | O | ARG | M | 1268 | 73.990 | 113.251 | 46.939 | 1.00 | 43.93 | 8 |
| ATOM | 18685 | N | PRO | M | 1269 | 73.152 | 112.019 | 48.617 | 1.00 | 61.56 | 7 |
| ATOM | 18686 | CD | PRO | M | 1269 | 73.394 | 111.513 | 49.971 | 1.00 | 19.49 | 6 |
| ATOM | 18687 | CA | PRO | M | 1269 | 71.715 | 112.276 | 48.458 | 1.00 | 61.86 | 6 |
| ATOM | 18688 | CB | PRO | M | 1269 | 71.133 | 111.631 | 49.686 | 1.00 | 20.65 | 6 |
| ATOM | 18689 | CG | PRO | M | 1269 | 72.167 | 111.953 | 50.678 | 1.00 | 19.82 | 6 |
| ATOM | 18690 | C | PRO | M | 1269 | 71.267 | 113.722 | 48.374 | 1.00 | 62.20 | 6 |
| ATOM | 18691 | O | PRO | M | 1269 | 72.081 | 114.643 | 48.362 | 1.00 | 63.87 | 8 |
| ATOM | 18692 | N | LYS | M | 1270 | 69.947 | 113.900 | 48.355 | 1.00 | 64.12 | 7 |
| ATOM | 18693 | CA | LYS | M | 1270 | 69.346 | 115.224 | 48.279 | 1.00 | 63.81 | 6 |
| ATOM | 18694 | CB | LYS | M | 1270 | 67.819 | 115.139 | 48.375 | 1.00 | 92.45 | 6 |
| ATOM | 18695 | CG | LYS | M | 1270 | 67.130 | 116.503 | 48.360 | 1.00 | 93.83 | 6 |
| ATOM | 18696 | CD | LYS | M | 1270 | 65.647 | 116.411 | 48.686 | 1.00 | 94.46 | 6 |
| ATOM | 18697 | CE | LYS | M | 1270 | 65.036 | 117.802 | 48.806 | 1.00 | 95.14 | 6 |
| ATOM | 18698 | NZ | LYS | M | 1270 | 63.612 | 117.811 | 49.256 | 1.00 | 96.33 | 7 |
| ATOM | 18699 | C | LYS | M | 1270 | 69.864 | 116.137 | 49.388 | 1.00 | 62.75 | 6 |
| ATOM | 18700 | O | LYS | M | 1270 | 70.511 | 117.154 | 49.121 | 1.00 | 62.13 | 8 |
| ATOM | 18701 | N | ALA | M | 1271 | 69.582 | 115.772 | 50.632 | 1.00 | 34.64 | 7 |
| ATOM | 18702 | CA | ALA | M | 1271 | 70.014 | 116.572 | 51.770 | 1.00 | 31.35 | 6 |
| ATOM | 18703 | CB | ALA | M | 1271 | 69.802 | 115.787 | 53.051 | 1.00 | 47.33 | 6 |
| ATOM | 18704 | C | ALA | M | 1271 | 71.462 | 117.039 | 51.692 | 1.00 | 29.53 | 6 |
| ATOM | 18705 | O | ALA | M | 1271 | 71.730 | 118.229 | 51.579 | 1.00 | 28.66 | 8 |
| ATOM | 18706 | N | ALA | M | 1272 | 72.388 | 116.089 | 51.753 | 1.00 | 45.65 | 7 |
| ATOM | 18707 | CA | ALA | M | 1272 | 73.813 | 116.392 | 51.727 | 1.00 | 45.37 | 6 |
| ATOM | 18708 | CB | ALA | M | 1272 | 74.160 | 117.172 | 50.470 | 1.00 | 44.00 | 6 |
| ATOM | 18709 | C | ALA | M | 1272 | 74.136 | 117.210 | 52.986 | 1.00 | 44.52 | 6 |
| ATOM | 18710 | O | ALA | M | 1272 | 75.193 | 117.831 | 53.094 | 1.00 | 43.90 | 8 |
| ATOM | 18711 | N | ALA | M | 1273 | 73.203 | 117.178 | 53.934 | 1.00 | 36.55 | 7 |
| ATOM | 18712 | CA | ALA | M | 1273 | 73.306 | 117.878 | 55.214 | 1.00 | 36.06 | 6 |
| ATOM | 18713 | CB | ALA | M | 1273 | 72.439 | 117.150 | 56.258 | 1.00 | 37.25 | 6 |
| ATOM | 18714 | C | ALA | M | 1273 | 74.738 | 117.984 | 55.717 | 1.00 | 35.72 | 6 |
| ATOM | 18715 | O | ALA | M | 1273 | 75.290 | 116.991 | 56.171 | 1.00 | 36.51 | 8 |
| ATOM | 18716 | N | VAL | M | 1274 | 75.335 | 119.176 | 55.660 | 1.00 | 36.79 | 7 |
| ATOM | 18717 | CA | VAL | M | 1274 | 76.725 | 119.351 | 56.121 | 1.00 | 37.99 | 6 |
| ATOM | 18718 | CB | VAL | M | 1274 | 77.232 | 120.821 | 56.015 | 1.00 | 53.09 | 6 |
| ATOM | 18719 | CG1 | VAL | M | 1274 | 78.654 | 120.805 | 55.484 | 1.00 | 54.50 | 6 |
| ATOM | 18720 | CG2 | VAL | M | 1274 | 76.340 | 121.665 | 55.118 | 1.00 | 53.07 | 6 |
| ATOM | 18721 | C | VAL | M | 1274 | 76.942 | 118.893 | 57.563 | 1.00 | 37.44 | 6 |
| ATOM | 18722 | O | VAL | M | 1274 | 76.051 | 119.008 | 58.409 | 1.00 | 37.19 | 8 |
| ATOM | 18723 | N | ILE | M | 1275 | 78.140 | 118.382 | 57.839 | 1.00 | 35.91 | 7 |
| ATOM | 18724 | CA | ILE | M | 1275 | 78.459 | 117.873 | 59.168 | 1.00 | 38.90 | 6 |
| ATOM | 18725 | CB | ILE | M | 1275 | 78.560 | 116.324 | 59.158 | 1.00 | 62.31 | 6 |
| ATOM | 18726 | CG2 | ILE | M | 1275 | 77.233 | 115.727 | 59.584 | 1.00 | 63.25 | 6 |
| ATOM | 18727 | CG1 | ILE | M | 1275 | 78.937 | 115.812 | 57.760 | 1.00 | 63.17 | 6 |
| ATOM | 18728 | CD1 | ILE | M | 1275 | 80.242 | 116.339 | 57.229 | 1.00 | 64.53 | 6 |
| ATOM | 18729 | C | ILE | M | 1275 | 79.735 | 118.432 | 59.758 | 1.00 | 38.86 | 6 |
| ATOM | 18730 | O | ILE | M | 1275 | 80.831 | 118.168 | 59.260 | 1.00 | 38.84 | 8 |
| ATOM | 18731 | N | SER | M | 1276 | 79.590 | 119.196 | 60.835 | 1.00 | 24.22 | 7 |
| ATOM | 18732 | CA | SER | M | 1276 | 80.741 | 119.806 | 61.491 | 1.00 | 26.41 | 6 |
| ATOM | 18733 | CB | SER | M | 1276 | 80.362 | 120.285 | 62.893 | 1.00 | 91.16 | 6 |
| ATOM | 18734 | OG | SER | M | 1276 | 81.423 | 121.009 | 63.487 | 1.00 | 95.56 | 8 |
| ATOM | 18735 | C | SER | M | 1276 | 81.914 | 118.827 | 61.568 | 1.00 | 26.43 | 6 |
| ATOM | 18736 | O | SER | M | 1276 | 81.778 | 117.718 | 62.080 | 1.00 | 25.83 | 8 |
| ATOM | 18737 | N | GLU | M | 1277 | 83.061 | 119.245 | 61.041 | 1.00 | 35.97 | 7 |
| ATOM | 18738 | CA | GLU | M | 1277 | 84.258 | 118.416 | 61.037 | 1.00 | 38.59 | 6 |
| ATOM | 18739 | CB | GLU | M | 1277 | 85.144 | 118.815 | 59.858 | 1.00 | 90.83 | 6 |
| ATOM | 18740 | CG | GLU | M | 1277 | 84.360 | 119.071 | 58.581 | 1.00 | 93.32 | 6 |
| ATOM | 18741 | CD | GLU | M | 1277 | 85.250 | 119.222 | 57.364 | 1.00 | 95.17 | 6 |
| ATOM | 18742 | OE1 | GLU | M | 1277 | 84.763 | 119.743 | 56.341 | 1.00 | 98.25 | 8 |
| ATOM | 18743 | OE2 | GLU | M | 1277 | 86.431 | 118.816 | 57.427 | 1.00 | 95.08 | 8 |
| ATOM | 18744 | C | GLU | M | 1277 | 85.040 | 118.524 | 62.357 | 1.00 | 39.76 | 6 |
| ATOM | 18745 | O | GLU | M | 1277 | 85.962 | 117.750 | 62.606 | 1.00 | 39.25 | 8 |
| ATOM | 18746 | N | ILE | M | 1278 | 84.664 | 119.485 | 63.196 | 1.00 | 59.13 | 7 |
| ATOM | 18747 | CA | ILE | M | 1278 | 85.308 | 119.669 | 64.492 | 1.00 | 60.77 | 6 |
| ATOM | 18748 | CB | ILE | M | 1278 | 85.733 | 121.111 | 64.743 | 1.00 | 48.99 | 6 |
| ATOM | 18749 | CG2 | ILE | M | 1278 | 86.689 | 121.154 | 65.914 | 1.00 | 49.48 | 6 |
| ATOM | 18750 | CG1 | ILE | M | 1278 | 86.393 | 121.702 | 63.516 | 1.00 | 49.01 | 6 |
| ATOM | 18751 | CD1 | ILE | M | 1278 | 86.653 | 123.184 | 63.677 | 1.00 | 49.31 | 6 |
| ATOM | 18752 | C | ILE | M | 1278 | 84.266 | 119.388 | 65.557 | 1.00 | 61.31 | 6 |
| ATOM | 18753 | O | ILE | M | 1278 | 83.082 | 119.311 | 65.256 | 1.00 | 62.17 | 8 |
| ATOM | 18754 | N | ASP | M | 1279 | 84.706 | 119.223 | 66.799 | 1.00 | 95.73 | 7 |
| ATOM | 18755 | CA | ASP | M | 1279 | 83.764 | 119.030 | 67.883 | 1.00 | 96.52 | 6 |
| ATOM | 18756 | CB | ASP | M | 1279 | 84.324 | 118.128 | 68.987 | 1.00 | 119.40 | 6 |
| ATOM | 18757 | CG | ASP | M | 1279 | 85.803 | 117.870 | 68.844 | 1.00 | 121.23 | 6 |
| ATOM | 18758 | OD1 | ASP | M | 1279 | 86.219 | 117.271 | 67.826 | 1.00 | 121.73 | 8 |
| ATOM | 18759 | OD2 | ASP | M | 1279 | 86.551 | 118.263 | 69.762 | 1.00 | 122.14 | 8 |
| ATOM | 18760 | C | ASP | M | 1279 | 83.601 | 120.450 | 68.376 | 1.00 | 96.37 | 6 |
| ATOM | 18761 | O | ASP | M | 1279 | 82.947 | 120.711 | 69.386 | 1.00 | 98.12 | 8 |
| ATOM | 18762 | N | GLY | M | 1280 | 84.208 | 121.358 | 67.612 | 1.00 | 18.28 | 7 |
| ATOM | 18763 | CA | GLY | M | 1280 | 84.189 | 122.785 | 67.902 | 1.00 | 17.31 | 6 |
| ATOM | 18764 | C | GLY | M | 1280 | 82.995 | 123.529 | 68.520 | 1.00 | 16.53 | 6 |
| ATOM | 18765 | O | GLY | M | 1280 | 82.131 | 122.948 | 69.183 | 1.00 | 15.06 | 8 |
| ATOM | 18766 | N | VAL | M | 1281 | 82.947 | 124.841 | 68.267 | 1.00 | 35.72 | 7 |
| ATOM | 18767 | CA | VAL | M | 1281 | 81.923 | 125.704 | 68.840 | 1.00 | 37.74 | 6 |
| ATOM | 18768 | CB | VAL | M | 1281 | 82.541 | 126.445 | 70.046 | 1.00 | 95.01 | 6 |
| ATOM | 18769 | CG1 | VAL | M | 1281 | 81.505 | 127.221 | 70.786 | 1.00 | 95.60 | 6 |
| ATOM | 18770 | CG2 | VAL | M | 1281 | 83.204 | 125.441 | 70.980 | 1.00 | 96.27 | 6 |
| ATOM | 18771 | C | VAL | M | 1281 | 81.251 | 126.732 | 67.907 | 1.00 | 39.27 | 6 |
| ATOM | 18772 | O | VAL | M | 1281 | 81.659 | 126.927 | 66.760 | 1.00 | 38.88 | 8 |
| ATOM | 18773 | N | VAL | M | 1282 | 80.204 | 127.359 | 68.451 | 1.00 | 59.53 | 7 |
| ATOM | 18774 | CA | VAL | M | 1282 | 79.359 | 128.413 | 67.851 | 1.00 | 61.86 | 6 |
| ATOM | 18775 | CB | VAL | M | 1282 | 79.665 | 129.813 | 68.503 | 1.00 | 151.80 | 6 |
| ATOM | 18776 | CG1 | VAL | M | 1282 | 78.438 | 130.725 | 68.403 | 1.00 | 152.73 | 6 |
| ATOM | 18777 | CG2 | VAL | M | 1282 | 80.106 | 129.662 | 69.935 | 1.00 | 152.82 | 6 |
| ATOM | 18778 | C | VAL | M | 1282 | 79.324 | 128.682 | 66.345 | 1.00 | 62.54 | 6 |
| ATOM | 18779 | O | VAL | M | 1282 | 79.164 | 127.784 | 65.520 | 1.00 | 61.75 | 8 |
| ATOM | 18780 | N | ARG | M | 1283 | 79.435 | 129.987 | 66.070 | 1.00 | 98.63 | 7 |
| ATOM | 18781 | CA | ARG | M | 1283 | 79.467 | 130.688 | 64.783 | 1.00 | 100.02 | 6 |
| ATOM | 18782 | CB | ARG | M | 1283 | 80.839 | 130.549 | 64.133 | 1.00 | 92.86 | 6 |
| ATOM | 18783 | CG | ARG | M | 1283 | 81.221 | 131.785 | 63.340 | 1.00 | 91.12 | 6 |
| ATOM | 18784 | CD | ARG | M | 1283 | 81.274 | 133.019 | 64.232 | 1.00 | 89.92 | 6 |
| ATOM | 18785 | NE | ARG | M | 1283 | 81.475 | 134.230 | 63.447 | 1.00 | 89.26 | 7 |
| ATOM | 18786 | CZ | ARG | M | 1283 | 82.586 | 134.500 | 62.772 | 1.00 | 88.80 | 6 |
| ATOM | 18787 | NH1 | ARG | M | 1283 | 83.605 | 133.648 | 62.795 | 1.00 | 89.04 | 7 |
| ATOM | 18788 | NH2 | ARG | M | 1283 | 82.668 | 135.607 | 62.047 | 1.00 | 88.44 | 7 |
| ATOM | 18789 | C | ARG | M | 1283 | 78.421 | 130.503 | 63.709 | 1.00 | 101.02 | 6 |
| ATOM | 18790 | O | ARG | M | 1283 | 77.895 | 129.418 | 63.495 | 1.00 | 101.79 | 8 |
| ATOM | 18791 | N | ILE | M | 1284 | 78.170 | 131.618 | 63.026 | 1.00 | 41.68 | 7 |
| ATOM | 18792 | CA | ILE | M | 1284 | 77.211 | 131.754 | 61.931 | 1.00 | 43.84 | 6 |
| ATOM | 18793 | CB | ILE | M | 1284 | 75.756 | 131.414 | 62.370 | 1.00 | 73.81 | 6 |
| ATOM | 18794 | CG2 | ILE | M | 1284 | 74.750 | 132.221 | 61.549 | 1.00 | 73.25 | 6 |
| ATOM | 18795 | CG1 | ILE | M | 1284 | 75.508 | 129.906 | 62.212 | 1.00 | 74.30 | 6 |
| ATOM | 18796 | CD1 | ILE | M | 1284 | 74.139 | 129.421 | 62.679 | 1.00 | 74.58 | 6 |
| ATOM | 18797 | C | ILE | M | 1284 | 77.263 | 133.220 | 61.528 | 1.00 | 45.61 | 6 |
| ATOM | 18798 | O | ILE | M | 1284 | 77.201 | 134.089 | 62.397 | 1.00 | 45.94 | 8 |
| ATOM | 18799 | N | GLU | M | 1285 | 77.383 | 133.488 | 60.225 | 1.00 | 81.27 | 7 |
| ATOM | 18800 | CA | GLU | M | 1285 | 77.455 | 134.859 | 59.697 | 1.00 | 84.47 | 6 |
| ATOM | 18801 | CB | GLU | M | 1285 | 78.901 | 135.226 | 59.343 | 1.00 | 145.08 | 6 |
| ATOM | 18802 | CG | GLU | M | 1285 | 79.848 | 135.342 | 60.527 | 1.00 | 148.56 | 6 |
| ATOM | 18803 | CD | GLU | M | 1285 | 79.437 | 136.432 | 61.494 | 1.00 | 150.99 | 6 |
| ATOM | 18804 | OE1 | GLU | M | 1285 | 79.228 | 137.577 | 61.042 | 1.00 | 152.74 | 8 |
| ATOM | 18805 | OE2 | GLU | M | 1285 | 79.331 | 136.147 | 62.706 | 1.00 | 152.74 | 8 |
| ATOM | 18806 | C | GLU | M | 1285 | 76.591 | 135.036 | 58.452 | 1.00 | 85.61 | 6 |
| ATOM | 18807 | O | GLU | M | 1285 | 76.945 | 134.566 | 57.372 | 1.00 | 85.94 | 8 |
| ATOM | 18808 | N | GLU | M | 1286 | 75.471 | 135.737 | 58.609 | 1.00 | 113.24 | 7 |
| ATOM | 18809 | CA | GLU | M | 1286 | 74.544 | 135.976 | 57.504 | 1.00 | 114.27 | 6 |
| ATOM | 18810 | CB | GLU | M | 1286 | 73.220 | 136.562 | 58.012 | 1.00 | 131.82 | 6 |
| ATOM | 18811 | CG | GLU | M | 1286 | 72.253 | 136.934 | 56.879 | 1.00 | 133.69 | 6 |
| ATOM | 18812 | CD | GLU | M | 1286 | 71.074 | 137.783 | 57.339 | 1.00 | 134.22 | 6 |
| ATOM | 18813 | OE1 | GLU | M | 1286 | 70.298 | 137.314 | 58.194 | 1.00 | 133.52 | 8 |
| ATOM | 18814 | OE2 | GLU | M | 1286 | 70.921 | 138.920 | 56.841 | 1.00 | 134.82 | 8 |
| ATOM | 18815 | C | GLU | M | 1286 | 75.070 | 136.897 | 56.415 | 1.00 | 114.18 | 6 |
| ATOM | 18816 | O | GLU | M | 1286 | 75.340 | 138.074 | 56.647 | 1.00 | 114.30 | 8 |
| ATOM | 18817 | N | GLY | M | 1287 | 75.199 | 136.347 | 55.219 | 1.00 | 127.50 | 7 |
| ATOM | 18818 | CA | GLY | M | 1287 | 75.643 | 137.131 | 54.088 | 1.00 | 128.07 | 6 |
| ATOM | 18819 | C | GLY | M | 1287 | 74.478 | 137.104 | 53.123 | 1.00 | 128.48 | 6 |
| ATOM | 18820 | O | GLY | M | 1287 | 73.755 | 136.108 | 53.070 | 1.00 | 128.72 | 8 |
| ATOM | 18821 | N | ALA | M | 1288 | 74.272 | 138.180 | 52.373 | 1.00 | 152.12 | 7 |
| ATOM | 18822 | CA | ALA | M | 1288 | 73.168 | 138.226 | 51.419 | 1.00 | 152.14 | 6 |
| ATOM | 18823 | CB | ALA | M | 1288 | 73.392 | 139.354 | 50.404 | 1.00 | 154.91 | 6 |
| ATOM | 18824 | C | ALA | M | 1288 | 73.029 | 136.883 | 50.696 | 1.00 | 151.69 | 6 |
| ATOM | 18825 | O | ALA | M | 1288 | 71.920 | 136.410 | 50.443 | 1.00 | 151.82 | 8 |
| ATOM | 18826 | N | ASP | M | 1289 | 74.166 | 136.267 | 50.389 | 1.00 | 103.92 | 7 |
| ATOM | 18827 | CA | ASP | M | 1289 | 74.194 | 134.988 | 49.692 | 1.00 | 102.41 | 6 |
| ATOM | 18828 | CB | ASP | M | 1289 | 74.495 | 135.218 | 48.205 | 1.00 | 56.26 | 6 |
| ATOM | 18829 | CG | ASP | M | 1289 | 73.252 | 135.566 | 47.388 | 1.00 | 54.29 | 6 |
| ATOM | 18830 | OD1 | ASP | M | 1289 | 72.430 | 134.658 | 47.145 | 1.00 | 53.95 | 8 |
| ATOM | 18831 | OD2 | ASP | M | 1289 | 73.101 | 136.741 | 46.987 | 1.00 | 53.57 | 8 |
| ATOM | 18832 | C | ASP | M | 1289 | 75.243 | 134.046 | 50.281 | 1.00 | 102.06 | 6 |
| ATOM | 18833 | O | ASP | M | 1289 | 75.882 | 133.300 | 49.547 | 1.00 | 102.17 | 8 |
| ATOM | 18834 | N | ARG | M | 1290 | 75.416 | 134.078 | 51.601 | 1.00 | 177.42 | 7 |
| ATOM | 18835 | CA | ARG | M | 1290 | 76.397 | 133.220 | 52.267 | 1.00 | 176.35 | 6 |
| ATOM | 18836 | CB | ARG | M | 1290 | 77.804 | 133.826 | 52.140 | 1.00 | 111.88 | 6 |
| ATOM | 18837 | CG | ARG | M | 1290 | 78.360 | 133.864 | 50.726 | 1.00 | 112.71 | 6 |
| ATOM | 18838 | CD | ARG | M | 1290 | 78.532 | 132.452 | 50.178 | 1.00 | 114.01 | 6 |
| ATOM | 18839 | NE | ARG | M | 1290 | 78.809 | 132.437 | 48.744 | 1.00 | 115.25 | 7 |
| ATOM | 18840 | CZ | ARG | M | 1290 | 78.931 | 131.332 | 48.015 | 1.00 | 115.11 | 6 |
| ATOM | 18841 | NH1 | ARG | M | 1290 | 78.802 | 130.138 | 48.576 | 1.00 | 115.12 | 7 |
| ATOM | 18842 | NH2 | ARG | M | 1290 | 79.176 | 131.423 | 46.718 | 1.00 | 115.37 | 7 |
| ATOM | 18843 | C | ARG | M | 1290 | 76.090 | 133.005 | 53.752 | 1.00 | 174.64 | 6 |
| ATOM | 18844 | O | ARG | M | 1290 | 76.438 | 133.852 | 54.577 | 1.00 | 174.46 | 8 |
| ATOM | 18845 | N | LEU | M | 1291 | 75.451 | 131.886 | 54.103 | 1.00 | 53.05 | 7 |
| ATOM | 18846 | CA | LEU | M | 1291 | 75.147 | 131.627 | 55.515 | 1.00 | 50.77 | 6 |
| ATOM | 18847 | CB | LEU | M | 1291 | 74.052 | 130.564 | 55.663 | 1.00 | 153.51 | 6 |
| ATOM | 18848 | CG | LEU | M | 1291 | 72.652 | 131.090 | 56.006 | 1.00 | 154.05 | 6 |
| ATOM | 18849 | CD1 | LEU | M | 1291 | 72.059 | 131.822 | 54.809 | 1.00 | 154.44 | 6 |
| ATOM | 18850 | CD2 | LEU | M | 1291 | 71.757 | 129.931 | 56.416 | 1.00 | 155.06 | 6 |
| ATOM | 18851 | C | LEU | M | 1291 | 76.400 | 131.187 | 56.280 | 1.00 | 49.06 | 6 |
| ATOM | 18852 | O | LEU | M | 1291 | 76.445 | 131.216 | 57.519 | 1.00 | 48.84 | 8 |
| ATOM | 18853 | N | SER | M | 1292 | 77.410 | 130.788 | 55.514 | 1.00 | 102.48 | 7 |
| ATOM | 18854 | CA | SER | M | 1292 | 78.695 | 130.339 | 56.035 | 1.00 | 101.03 | 6 |
| ATOM | 18855 | CB | SER | M | 1292 | 79.778 | 131.354 | 55.658 | 1.00 | 138.13 | 6 |
| ATOM | 18856 | OG | SER | M | 1292 | 79.417 | 132.655 | 56.087 | 1.00 | 141.06 | 8 |
| ATOM | 18857 | C | SER | M | 1292 | 78.748 | 130.065 | 57.538 | 1.00 | 98.69 | 6 |
| ATOM | 18858 | O | SER | M | 1292 | 79.463 | 130.745 | 58.267 | 1.00 | 99.06 | 8 |
| ATOM | 18859 | N | VAL | M | 1293 | 77.986 | 129.073 | 57.994 | 1.00 | 37.22 | 7 |
| ATOM | 18860 | CA | VAL | M | 1293 | 77.973 | 128.683 | 59.405 | 1.00 | 33.89 | 6 |
| ATOM | 18861 | CB | VAL | M | 1293 | 77.025 | 127.480 | 59.652 | 1.00 | 24.86 | 6 |
| ATOM | 18862 | CG1 | VAL | M | 1293 | 77.162 | 126.998 | 61.069 | 1.00 | 23.47 | 6 |
| ATOM | 18863 | CG2 | VAL | M | 1293 | 75.594 | 127.860 | 59.372 | 1.00 | 24.56 | 6 |
| ATOM | 18864 | C | VAL | M | 1293 | 79.386 | 128.222 | 59.763 | 1.00 | 33.09 | 6 |
| ATOM | 18865 | O | VAL | M | 1293 | 79.828 | 127.192 | 59.260 | 1.00 | 32.16 | 8 |
| ATOM | 18866 | N | PHE | M | 1294 | 80.099 | 128.972 | 60.605 | 1.00 | 59.03 | 7 |
| ATOM | 18867 | CA | PHE | M | 1294 | 81.453 | 128.566 | 60.981 | 1.00 | 58.35 | 6 |
| ATOM | 18868 | CB | PHE | M | 1294 | 82.343 | 129.738 | 61.401 | 1.00 | 99.37 | 6 |
| ATOM | 18869 | CG | PHE | M | 1294 | 82.564 | 130.785 | 60.356 | 1.00 | 100.89 | 6 |
| ATOM | 18870 | CD1 | PHE | M | 1294 | 81.513 | 131.563 | 59.890 | 1.00 | 101.42 | 6 |
| ATOM | 18871 | CD2 | PHE | M | 1294 | 83.851 | 131.071 | 59.917 | 1.00 | 101.34 | 6 |
| ATOM | 18872 | CE1 | PHE | M | 1294 | 81.742 | 132.621 | 59.009 | 1.00 | 101.34 | 6 |
| ATOM | 18873 | CE2 | PHE | M | 1294 | 84.091 | 132.123 | 59.040 | 1.00 | 101.32 | 6 |
| ATOM | 18874 | CZ | PHE | M | 1294 | 83.035 | 132.902 | 58.585 | 1.00 | 101.85 | 6 |
| ATOM | 18875 | C | PHE | M | 1294 | 81.397 | 127.664 | 62.192 | 1.00 | 58.03 | 6 |
| ATOM | 18876 | O | PHE | M | 1294 | 80.399 | 127.619 | 62.899 | 1.00 | 58.40 | 8 |
| ATOM | 18877 | N | VAL | M | 1295 | 82.496 | 126.957 | 62.420 | 1.00 | 42.46 | 7 |
| ATOM | 18878 | CA | VAL | M | 1295 | 82.675 | 126.089 | 63.578 | 1.00 | 42.27 | 6 |
| ATOM | 18879 | CB | VAL | M | 1295 | 82.673 | 124.601 | 63.221 | 1.00 | 81.31 | 6 |
| ATOM | 18880 | CG1 | VAL | M | 1295 | 83.464 | 123.816 | 64.265 | 1.00 | 81.68 | 6 |
| ATOM | 18881 | CG2 | VAL | M | 1295 | 81.252 | 124.090 | 63.175 | 1.00 | 82.09 | 6 |
| ATOM | 18882 | C | VAL | M | 1295 | 84.081 | 126.478 | 63.952 | 1.00 | 42.78 | 6 |
| ATOM | 18883 | O | VAL | M | 1295 | 84.895 | 126.703 | 63.058 | 1.00 | 43.54 | 8 |
| ATOM | 18884 | N | GLU | M | 1296 | 84.385 | 126.577 | 65.243 | 1.00 | 57.95 | 7 |
| ATOM | 18885 | CA | GLU | M | 1296 | 85.735 | 126.972 | 65.626 | 1.00 | 58.50 | 6 |
| ATOM | 18886 | CB | GLU | M | 1296 | 85.979 | 128.433 | 65.228 | 1.00 | 53.08 | 6 |
| ATOM | 18887 | CG | GLU | M | 1296 | 85.075 | 129.430 | 65.944 | 1.00 | 51.32 | 6 |
| ATOM | 18888 | CD | GLU | M | 1296 | 84.901 | 130.742 | 65.183 | 1.00 | 50.83 | 6 |
| ATOM | 18889 | OE1 | GLU | M | 1296 | 84.295 | 131.680 | 65.744 | 1.00 | 50.78 | 8 |
| ATOM | 18890 | OE2 | GLU | M | 1296 | 85.354 | 130.838 | 64.022 | 1.00 | 49.13 | 8 |
| ATOM | 18891 | C | GLU | M | 1296 | 86.062 | 126.807 | 67.091 | 1.00 | 59.33 | 6 |
| ATOM | 18892 | O | GLU | M | 1296 | 85.259 | 127.138 | 67.961 | 1.00 | 58.10 | 8 |
| ATOM | 18893 | N | SER | M | 1297 | 87.255 | 126.284 | 67.348 | 1.00 | 64.61 | 7 |
| ATOM | 18894 | CA | SER | M | 1297 | 87.745 | 126.107 | 68.707 | 1.00 | 68.46 | 6 |
| ATOM | 18895 | CB | SER | M | 1297 | 88.637 | 124.861 | 68.810 | 1.00 | 113.52 | 6 |
| ATOM | 18896 | OG | SER | M | 1297 | 87.894 | 123.660 | 68.675 | 1.00 | 116.51 | 8 |
| ATOM | 18897 | C | SER | M | 1297 | 88.583 | 127.352 | 68.971 | 1.00 | 70.65 | 6 |
| ATOM | 18898 | O | SER | M | 1297 | 88.364 | 128.392 | 68.357 | 1.00 | 71.33 | 8 |
| ATOM | 18899 | N | GLU | M | 1298 | 89.528 | 127.256 | 69.895 | 1.00 | 82.71 | 7 |
| ATOM | 18900 | CA | GLU | M | 1298 | 90.413 | 128.375 | 70.159 | 1.00 | 84.60 | 6 |
| ATOM | 18901 | CB | GLU | M | 1298 | 90.840 | 128.403 | 71.625 | 1.00 | 116.20 | 6 |
| ATOM | 18902 | CG | GLU | M | 1298 | 91.751 | 127.258 | 72.014 | 1.00 | 118.49 | 6 |
| ATOM | 18903 | CD | GLU | M | 1298 | 92.100 | 127.279 | 73.482 | 1.00 | 120.31 | 6 |
| ATOM | 18904 | OE1 | GLU | M | 1298 | 91.161 | 127.255 | 74.310 | 1.00 | 119.43 | 8 |
| ATOM | 18905 | OE2 | GLU | M | 1298 | 93.309 | 127.315 | 73.806 | 1.00 | 123.17 | 8 |
| ATOM | 18906 | C | GLU | M | 1298 | 91.599 | 128.023 | 69.277 | 1.00 | 85.69 | 6 |
| ATOM | 18907 | O | GLU | M | 1298 | 92.488 | 128.834 | 69.049 | 1.00 | 86.59 | 8 |
| ATOM | 18908 | N | GLY | M | 1299 | 91.579 | 126.791 | 68.778 | 1.00 | 52.28 | 7 |
| ATOM | 18909 | CA | GLY | M | 1299 | 92.642 | 126.298 | 67.924 | 1.00 | 52.99 | 6 |
| ATOM | 18910 | C | GLY | M | 1299 | 92.507 | 126.607 | 66.445 | 1.00 | 53.87 | 6 |
| ATOM | 18911 | O | GLY | M | 1299 | 93.512 | 126.744 | 65.764 | 1.00 | 54.31 | 8 |
| ATOM | 18912 | N | PHE | M | 1300 | 91.281 | 126.692 | 65.940 | 1.00 | 46.42 | 7 |
| ATOM | 18913 | CA | PHE | M | 1300 | 91.037 | 127.009 | 64.532 | 1.00 | 49.22 | 6 |
| ATOM | 18914 | CB | PHE | M | 1300 | 91.683 | 125.972 | 63.602 | 1.00 | 110.85 | 6 |
| ATOM | 18915 | CG | PHE | M | 1300 | 91.422 | 124.546 | 63.991 | 1.00 | 113.85 | 6 |
| ATOM | 18916 | CD1 | PHE | M | 1300 | 90.132 | 124.098 | 64.235 | 1.00 | 115.65 | 6 |
| ATOM | 18917 | CD2 | PHE | M | 1300 | 92.472 | 123.640 | 64.085 | 1.00 | 115.60 | 6 |
| ATOM | 18918 | CE1 | PHE | M | 1300 | 89.891 | 122.767 | 64.566 | 1.00 | 117.56 | 6 |
| ATOM | 18919 | CE2 | PHE | M | 1300 | 92.241 | 122.310 | 64.415 | 1.00 | 117.01 | 6 |
| ATOM | 18920 | CZ | PHE | M | 1300 | 90.948 | 121.873 | 64.655 | 1.00 | 117.75 | 6 |
| ATOM | 18921 | C | PHE | M | 1300 | 89.549 | 127.141 | 64.204 | 1.00 | 50.22 | 6 |
| ATOM | 18922 | O | PHE | M | 1300 | 88.685 | 126.905 | 65.056 | 1.00 | 49.97 | 8 |
| ATOM | 18923 | N | SER | M | 1301 | 89.253 | 127.520 | 62.965 | 1.00 | 85.62 | 7 |
| ATOM | 18924 | CA | SER | M | 1301 | 87.878 | 127.693 | 62.532 | 1.00 | 86.63 | 6 |
| ATOM | 18925 | CB | SER | M | 1301 | 87.617 | 129.164 | 62.215 | 1.00 | 125.65 | 6 |
| ATOM | 18926 | OG | SER | M | 1301 | 87.829 | 129.960 | 63.363 | 1.00 | 127.19 | 8 |
| ATOM | 18927 | C | SER | M | 1301 | 87.565 | 126.832 | 61.319 | 1.00 | 86.63 | 6 |
| ATOM | 18928 | O | SER | M | 1301 | 88.452 | 126.188 | 60.757 | 1.00 | 86.83 | 8 |
| ATOM | 18929 | N | LYS | M | 1302 | 86.295 | 126.831 | 60.925 | 1.00 | 65.54 | 7 |
| ATOM | 18930 | CA | LYS | M | 1302 | 85.823 | 126.046 | 59.790 | 1.00 | 66.43 | 6 |
| ATOM | 18931 | CB | LYS | M | 1302 | 85.272 | 124.710 | 60.287 | 1.00 | 142.22 | 6 |
| ATOM | 18932 | CG | LYS | M | 1302 | 85.263 | 123.599 | 59.260 | 1.00 | 143.00 | 6 |
| ATOM | 18933 | CD | LYS | M | 1302 | 86.625 | 122.950 | 59.145 | 1.00 | 145.79 | 6 |
| ATOM | 18934 | CE | LYS | M | 1302 | 86.574 | 121.756 | 58.211 | 1.00 | 148.24 | 6 |
| ATOM | 18935 | NZ | LYS | M | 1302 | 87.856 | 120.995 | 58.173 | 1.00 | 149.32 | 7 |
| ATOM | 18936 | C | LYS | M | 1302 | 84.708 | 126.824 | 59.098 | 1.00 | 66.84 | 6 |
| ATOM | 18937 | O | LYS | M | 1302 | 83.828 | 127.369 | 59.764 | 1.00 | 66.13 | 8 |
| ATOM | 18938 | N | GLU | M | 1303 | 84.747 | 126.879 | 57.769 | 1.00 | 97.90 | 7 |
| ATOM | 18939 | CA | GLU | M | 1303 | 83.721 | 127.590 | 57.006 | 1.00 | 98.72 | 6 |
| ATOM | 18940 | CB | GLU | M | 1303 | 84.364 | 128.545 | 55.997 | 1.00 | 169.03 | 6 |
| ATOM | 18941 | CG | GLU | M | 1303 | 85.008 | 129.767 | 56.628 | 1.00 | 169.15 | 6 |
| ATOM | 18942 | CD | GLU | M | 1303 | 85.583 | 130.725 | 55.603 | 1.00 | 170.20 | 6 |
| ATOM | 18943 | OE1 | GLU | M | 1303 | 86.517 | 130.332 | 54.872 | 1.00 | 170.40 | 8 |
| ATOM | 18944 | OE2 | GLU | M | 1303 | 85.101 | 131.873 | 55.525 | 1.00 | 170.55 | 8 |
| ATOM | 18945 | C | GLU | M | 1303 | 82.778 | 126.629 | 56.279 | 1.00 | 98.98 | 6 |
| ATOM | 18946 | O | GLU | M | 1303 | 83.201 | 125.580 | 55.788 | 1.00 | 100.07 | 8 |
| ATOM | 18947 | N | TYR | M | 1304 | 81.497 | 126.994 | 56.214 | 1.00 | 115.98 | 7 |
| ATOM | 18948 | CA | TYR | M | 1304 | 80.491 | 126.157 | 55.569 | 1.00 | 115.37 | 6 |
| ATOM | 18949 | CB | TYR | M | 1304 | 79.583 | 125.539 | 56.638 | 1.00 | 59.99 | 6 |
| ATOM | 18950 | CG | TYR | M | 1304 | 80.315 | 124.509 | 57.451 | 1.00 | 59.78 | 6 |
| ATOM | 18951 | CD1 | TYR | M | 1304 | 81.440 | 124.852 | 58.177 | 1.00 | 60.41 | 6 |
| ATOM | 18952 | CE1 | TYR | M | 1304 | 82.205 | 123.895 | 58.826 | 1.00 | 61.28 | 6 |
| ATOM | 18953 | CD2 | TYR | M | 1304 | 79.959 | 123.178 | 57.406 | 1.00 | 59.60 | 6 |
| ATOM | 18954 | CE2 | TYR | M | 1304 | 80.719 | 122.207 | 58.058 | 1.00 | 60.64 | 6 |
| ATOM | 18955 | CZ | TYR | M | 1304 | 81.844 | 122.574 | 58.760 | 1.00 | 61.10 | 6 |
| ATOM | 18956 | OH | TYR | M | 1304 | 82.639 | 121.625 | 59.355 | 1.00 | 63.64 | 8 |
| ATOM | 18957 | C | TYR | M | 1304 | 79.644 | 126.831 | 54.499 | 1.00 | 116.12 | 6 |
| ATOM | 18958 | O | TYR | M | 1304 | 78.767 | 127.635 | 54.797 | 1.00 | 116.18 | 8 |
| ATOM | 18959 | N | LYS | M | 1305 | 79.925 | 126.491 | 53.247 | 1.00 | 140.45 | 7 |
| ATOM | 18960 | CA | LYS | M | 1305 | 79.181 | 127.019 | 52.113 | 1.00 | 141.79 | 6 |
| ATOM | 18961 | CB | LYS | M | 1305 | 79.703 | 126.396 | 50.808 | 1.00 | 169.80 | 6 |
| ATOM | 18962 | CG | LYS | M | 1305 | 79.570 | 124.866 | 50.713 | 1.00 | 170.32 | 6 |
| ATOM | 18963 | CD | LYS | M | 1305 | 80.336 | 124.146 | 51.818 | 1.00 | 171.27 | 6 |
| ATOM | 18964 | CE | LYS | M | 1305 | 80.011 | 122.661 | 51.840 | 1.00 | 171.48 | 6 |
| ATOM | 18965 | NZ | LYS | M | 1305 | 80.697 | 121.959 | 52.959 | 1.00 | 172.56 | 7 |
| ATOM | 18966 | C | LYS | M | 1305 | 77.723 | 126.634 | 52.332 | 1.00 | 142.65 | 6 |
| ATOM | 18967 | O | LYS | M | 1305 | 77.270 | 125.591 | 51.860 | 1.00 | 143.43 | 8 |
| ATOM | 18968 | N | LEU | M | 1306 | 76.984 | 127.474 | 53.048 | 1.00 | 133.27 | 7 |
| ATOM | 18969 | CA | LEU | M | 1306 | 75.591 | 127.161 | 53.339 | 1.00 | 134.57 | 6 |
| ATOM | 18970 | CB | LEU | M | 1306 | 75.416 | 127.011 | 54.850 | 1.00 | 72.93 | 6 |
| ATOM | 18971 | CG | LEU | M | 1306 | 74.078 | 126.476 | 55.345 | 1.00 | 74.33 | 6 |
| ATOM | 18972 | CD1 | LEU | M | 1306 | 73.728 | 125.202 | 54.590 | 1.00 | 74.92 | 6 |
| ATOM | 18973 | CD2 | LEU | M | 1306 | 74.160 | 126.221 | 56.847 | 1.00 | 74.31 | 6 |
| ATOM | 18974 | C | LEU | M | 1306 | 74.557 | 128.146 | 52.798 | 1.00 | 135.34 | 6 |
| ATOM | 18975 | O | LEU | M | 1306 | 74.504 | 129.300 | 53.221 | 1.00 | 135.88 | 8 |
| ATOM | 18976 | N | PRO | M | 1307 | 73.736 | 127.699 | 51.830 | 1.00 | 74.34 | 7 |
| ATOM | 18977 | CD | PRO | M | 1307 | 74.106 | 126.586 | 50.946 | 1.00 | 129.51 | 6 |
| ATOM | 18978 | CA | PRO | M | 1307 | 72.680 | 128.500 | 51.203 | 1.00 | 74.47 | 6 |
| ATOM | 18979 | CB | PRO | M | 1307 | 72.743 | 128.075 | 49.733 | 1.00 | 130.28 | 6 |
| ATOM | 18980 | CG | PRO | M | 1307 | 74.028 | 127.251 | 49.614 | 1.00 | 130.06 | 6 |
| ATOM | 18981 | C | PRO | M | 1307 | 71.341 | 128.120 | 51.832 | 1.00 | 75.61 | 6 |
| ATOM | 18982 | O | PRO | M | 1307 | 71.300 | 127.285 | 52.735 | 1.00 | 76.06 | 8 |
| ATOM | 18983 | N | LYS | M | 1308 | 70.259 | 128.718 | 51.337 | 1.00 | 161.78 | 7 |
| ATOM | 18984 | CA | LYS | M | 1308 | 68.891 | 128.466 | 51.815 | 1.00 | 162.39 | 6 |
| ATOM | 18985 | CB | LYS | M | 1308 | 67.908 | 128.594 | 50.645 | 1.00 | 154.21 | 6 |
| ATOM | 18986 | CG | LYS | M | 1308 | 68.152 | 129.780 | 49.729 | 1.00 | 154.31 | 6 |
| ATOM | 18987 | CD | LYS | M | 1308 | 67.253 | 129.718 | 48.500 | 1.00 | 153.70 | 6 |
| ATOM | 18988 | CE | LYS | M | 1308 | 67.531 | 130.879 | 47.560 | 1.00 | 153.41 | 6 |
| ATOM | 18989 | NZ | LYS | M | 1308 | 66.651 | 130.838 | 46.364 | 1.00 | 153.40 | 7 |
| ATOM | 18990 | C | LYS | M | 1308 | 68.678 | 127.090 | 52.464 | 1.00 | 162.62 | 6 |
| ATOM | 18991 | O | LYS | M | 1308 | 68.168 | 126.173 | 51.820 | 1.00 | 163.21 | 8 |
| ATOM | 18992 | N | ALA | M | 1309 | 69.040 | 126.950 | 53.735 | 1.00 | 136.19 | 7 |
| ATOM | 18993 | CA | ALA | M | 1309 | 68.878 | 125.675 | 54.428 | 1.00 | 136.08 | 6 |
| ATOM | 18994 | CB | ALA | M | 1309 | 70.096 | 124.798 | 54.199 | 1.00 | 66.84 | 6 |
| ATOM | 18995 | C | ALA | M | 1309 | 68.664 | 125.881 | 55.919 | 1.00 | 135.99 | 6 |
| ATOM | 18996 | O | ALA | M | 1309 | 69.207 | 126.811 | 56.507 | 1.00 | 136.64 | 8 |
| ATOM | 18997 | N | ALA | M | 1310 | 67.878 | 124.998 | 56.525 | 1.00 | 106.87 | 7 |
| ATOM | 18998 | CA | ALA | M | 1310 | 67.573 | 125.072 | 57.951 | 1.00 | 106.10 | 6 |
| ATOM | 18999 | CB | ALA | M | 1310 | 66.575 | 123.978 | 58.307 | 1.00 | 147.46 | 6 |
| ATOM | 19000 | C | ALA | M | 1310 | 68.824 | 124.961 | 58.837 | 1.00 | 105.72 | 6 |
| ATOM | 19001 | O | ALA | M | 1310 | 69.120 | 123.900 | 59.385 | 1.00 | 106.16 | 8 |
| ATOM | 19002 | N | ALA | M | 1311 | 69.543 | 126.067 | 58.998 | 1.00 | 103.72 | 7 |
| ATOM | 19003 | CA | ALA | M | 1311 | 70.762 | 126.059 | 59.800 | 1.00 | 103.23 | 6 |
| ATOM | 19004 | CB | ALA | M | 1311 | 71.808 | 126.973 | 59.166 | 1.00 | 38.90 | 6 |
| ATOM | 19005 | C | ALA | M | 1311 | 70.564 | 126.442 | 61.258 | 1.00 | 103.42 | 6 |
| ATOM | 19006 | O | ALA | M | 1311 | 70.879 | 127.567 | 61.646 | 1.00 | 103.61 | 8 |
| ATOM | 19007 | N | LEU | M | 1312 | 70.067 | 125.508 | 62.069 | 1.00 | 181.17 | 7 |
| ATOM | 19008 | CA | LEU | M | 1312 | 69.845 | 125.778 | 63.490 | 1.00 | 180.81 | 6 |
| ATOM | 19009 | CB | LEU | M | 1312 | 69.559 | 124.476 | 64.250 | 1.00 | 186.51 | 6 |
| ATOM | 19010 | CG | LEU | M | 1312 | 68.198 | 123.805 | 64.035 | 1.00 | 187.60 | 6 |
| ATOM | 19011 | CD1 | LEU | M | 1312 | 68.016 | 123.415 | 62.578 | 1.00 | 187.30 | 6 |
| ATOM | 19012 | CD2 | LEU | M | 1312 | 68.106 | 122.582 | 64.927 | 1.00 | 188.28 | 6 |
| ATOM | 19013 | C | LEU | M | 1312 | 71.050 | 126.496 | 64.105 | 1.00 | 179.46 | 6 |
| ATOM | 19014 | O | LEU | M | 1312 | 72.124 | 125.919 | 64.267 | 1.00 | 179.00 | 8 |
| ATOM | 19015 | N | LEU | M | 1313 | 70.857 | 127.767 | 64.439 | 1.00 | 82.28 | 7 |
| ATOM | 19016 | CA | LEU | M | 1313 | 71.912 | 128.588 | 65.017 | 1.00 | 81.18 | 6 |
| ATOM | 19017 | CB | LEU | M | 1313 | 71.409 | 130.025 | 65.222 | 1.00 | 208.87 | 6 |
| ATOM | 19018 | CG | LEU | M | 1313 | 70.487 | 130.687 | 64.186 | 1.00 | 208.87 | 6 |
| ATOM | 19019 | CD1 | LEU | M | 1313 | 70.262 | 132.143 | 64.583 | 1.00 | 208.87 | 6 |
| ATOM | 19020 | CD2 | LEU | M | 1313 | 71.092 | 130.616 | 62.795 | 1.00 | 208.87 | 6 |
| ATOM | 19021 | C | LEU | M | 1313 | 72.369 | 128.023 | 66.354 | 1.00 | 80.04 | 6 |
| ATOM | 19022 | O | LEU | M | 1313 | 72.596 | 126.822 | 66.481 | 1.00 | 79.91 | 8 |
| ATOM | 19023 | N | ALA | M | 1314 | 72.501 | 128.917 | 67.333 | 1.00 | 141.50 | 7 |
| ATOM | 19024 | CA | ALA | M | 1314 | 72.917 | 128.597 | 68.693 | 1.00 | 140.89 | 6 |
| ATOM | 19025 | CB | ALA | M | 1314 | 72.582 | 127.148 | 69.032 | 1.00 | 136.16 | 6 |
| ATOM | 19026 | C | ALA | M | 1314 | 74.396 | 128.867 | 68.946 | 1.00 | 140.12 | 6 |
| ATOM | 19027 | O | ALA | M | 1314 | 74.909 | 129.933 | 68.592 | 1.00 | 140.20 | 8 |
| ATOM | 19028 | N | ALA | M | 1315 | 75.070 | 127.897 | 69.566 | 1.00 | 67.06 | 7 |
| ATOM | 19029 | CA | ALA | M | 1315 | 76.490 | 128.012 | 69.902 | 1.00 | 65.06 | 6 |
| ATOM | 19030 | CB | ALA | M | 1315 | 76.713 | 129.276 | 70.725 | 1.00 | 121.14 | 6 |
| ATOM | 19031 | C | ALA | M | 1315 | 77.048 | 126.781 | 70.649 | 1.00 | 63.78 | 6 |
| ATOM | 19032 | O | ALA | M | 1315 | 76.370 | 125.762 | 70.783 | 1.00 | 64.29 | 8 |
| ATOM | 19033 | N | ASP | M | 1316 | 78.285 | 126.904 | 71.132 | 1.00 | 77.21 | 7 |
| ATOM | 19034 | CA | ASP | M | 1316 | 78.999 | 125.841 | 71.848 | 1.00 | 76.28 | 6 |
| ATOM | 19035 | CB | ASP | M | 1316 | 78.762 | 125.949 | 73.383 | 1.00 | 39.27 | 6 |
| ATOM | 19036 | CG | ASP | M | 1316 | 79.983 | 126.538 | 74.151 | 1.00 | 36.80 | 6 |
| ATOM | 19037 | OD1 | ASP | M | 1316 | 81.081 | 125.944 | 74.152 | 1.00 | 36.63 | 8 |
| ATOM | 19038 | OD2 | ASP | M | 1316 | 79.855 | 127.609 | 74.766 | 1.00 | 35.24 | 8 |
| ATOM | 19039 | C | ASP | M | 1316 | 78.717 | 124.414 | 71.338 | 1.00 | 76.42 | 6 |
| ATOM | 19040 | O | ASP | M | 1316 | 77.698 | 124.141 | 70.694 | 1.00 | 76.45 | 8 |
| ATOM | 19041 | N | GLY | M | 1317 | 79.665 | 123.519 | 71.606 | 1.00 | 174.10 | 7 |
| ATOM | 19042 | CA | GLY | M | 1317 | 79.551 | 122.129 | 71.200 | 1.00 | 174.06 | 6 |
| ATOM | 19043 | C | GLY | M | 1317 | 79.249 | 121.876 | 69.735 | 1.00 | 173.96 | 6 |
| ATOM | 19044 | O | GLY | M | 1317 | 78.716 | 122.745 | 69.046 | 1.00 | 174.32 | 8 |
| ATOM | 19045 | N | ALA | M | 1318 | 79.600 | 120.677 | 69.271 | 1.00 | 93.21 | 7 |
| ATOM | 19046 | CA | ALA | M | 1318 | 79.378 | 120.226 | 67.892 | 1.00 | 92.30 | 6 |
| ATOM | 19047 | CB | ALA | M | 1318 | 79.327 | 121.421 | 66.919 | 1.00 | 95.94 | 6 |
| ATOM | 19048 | C | ALA | M | 1318 | 80.473 | 119.251 | 67.456 | 1.00 | 91.75 | 6 |
| ATOM | 19049 | O | ALA | M | 1318 | 81.432 | 119.658 | 66.813 | 1.00 | 91.28 | 8 |
| ATOM | 19050 | N | ALA | M | 1319 | 80.326 | 117.971 | 67.801 | 1.00 | 38.87 | 7 |
| ATOM | 19051 | CA | ALA | M | 1319 | 81.304 | 116.936 | 67.437 | 1.00 | 39.23 | 6 |
| ATOM | 19052 | CB | ALA | M | 1319 | 81.009 | 115.661 | 68.234 | 1.00 | 87.05 | 6 |
| ATOM | 19053 | C | ALA | M | 1319 | 81.334 | 116.620 | 65.924 | 1.00 | 39.49 | 6 |
| ATOM | 19054 | O | ALA | M | 1319 | 82.388 | 116.585 | 65.286 | 1.00 | 40.10 | 8 |
| ATOM | 19055 | N | VAL | M | 1320 | 80.152 | 116.375 | 65.376 | 1.00 | 72.64 | 7 |
| ATOM | 19056 | CA | VAL | M | 1320 | 79.943 | 116.064 | 63.969 | 1.00 | 73.01 | 6 |
| ATOM | 19057 | CB | VAL | M | 1320 | 80.241 | 114.586 | 63.668 | 1.00 | 176.14 | 6 |
| ATOM | 19058 | CG1 | VAL | M | 1320 | 79.897 | 114.278 | 62.230 | 1.00 | 177.46 | 6 |
| ATOM | 19059 | CG2 | VAL | M | 1320 | 81.698 | 114.272 | 63.937 | 1.00 | 176.43 | 6 |
| ATOM | 19060 | C | VAL | M | 1320 | 78.453 | 116.292 | 63.835 | 1.00 | 73.15 | 6 |
| ATOM | 19061 | O | VAL | M | 1320 | 77.924 | 116.579 | 62.760 | 1.00 | 73.64 | 8 |
| ATOM | 19062 | N | GLU | M | 1321 | 77.807 | 116.155 | 64.988 | 1.00 | 192.66 | 7 |
| ATOM | 19063 | CA | GLU | M | 1321 | 76.376 | 116.322 | 65.163 | 1.00 | 194.77 | 6 |
| ATOM | 19064 | CB | GLU | M | 1321 | 76.109 | 117.580 | 65.992 | 1.00 | 100.58 | 6 |
| ATOM | 19065 | CG | GLU | M | 1321 | 74.649 | 117.868 | 66.240 | 1.00 | 101.19 | 6 |
| ATOM | 19066 | CD | GLU | M | 1321 | 74.443 | 119.152 | 67.008 | 1.00 | 101.43 | 6 |
| ATOM | 19067 | OE1 | GLU | M | 1321 | 74.859 | 119.214 | 68.182 | 1.00 | 101.02 | 8 |
| ATOM | 19068 | OE2 | GLU | M | 1321 | 73.877 | 120.102 | 66.430 | 1.00 | 102.32 | 8 |
| ATOM | 19069 | C | GLU | M | 1321 | 75.615 | 116.392 | 63.856 | 1.00 | 195.66 | 6 |
| ATOM | 19070 | O | GLU | M | 1321 | 75.049 | 115.402 | 63.401 | 1.00 | 196.90 | 8 |
| ATOM | 19071 | N | ALA | M | 1322 | 75.626 | 117.565 | 63.242 | 1.00 | 36.01 | 7 |
| ATOM | 19072 | CA | ALA | M | 1322 | 74.913 | 117.764 | 62.007 | 1.00 | 36.83 | 6 |
| ATOM | 19073 | CB | ALA | M | 1322 | 73.446 | 117.750 | 62.283 | 1.00 | 39.61 | 6 |
| ATOM | 19074 | C | ALA | M | 1322 | 75.285 | 119.078 | 61.351 | 1.00 | 38.19 | 6 |
| ATOM | 19075 | O | ALA | M | 1322 | 74.402 | 119.804 | 60.907 | 1.00 | 39.21 | 8 |
| ATOM | 19076 | N | GLY | M | 1323 | 76.585 | 119.381 | 61.305 | 1.00 | 123.19 | 7 |
| ATOM | 19077 | CA | GLY | M | 1323 | 77.096 | 120.608 | 60.696 | 1.00 | 125.25 | 6 |
| ATOM | 19078 | C | GLY | M | 1323 | 76.300 | 121.877 | 60.944 | 1.00 | 126.30 | 6 |
| ATOM | 19079 | O | GLY | M | 1323 | 76.862 | 122.955 | 61.137 | 1.00 | 127.31 | 8 |
| ATOM | 19080 | N | GLN | M | 1324 | 74.985 | 121.712 | 60.909 | 1.00 | 59.99 | 7 |
| ATOM | 19081 | CA | GLN | M | 1324 | 73.968 | 122.720 | 61.112 | 1.00 | 61.95 | 6 |
| ATOM | 19082 | CB | GLN | M | 1324 | 74.544 | 123.989 | 61.749 | 1.00 | 179.79 | 6 |
| ATOM | 19083 | CG | GLN | M | 1324 | 74.458 | 123.966 | 63.279 | 1.00 | 180.04 | 6 |
| ATOM | 19084 | CD | GLN | M | 1324 | 73.139 | 123.376 | 63.777 | 1.00 | 179.63 | 6 |
| ATOM | 19085 | OE1 | GLN | M | 1324 | 72.059 | 123.771 | 63.334 | 1.00 | 180.03 | 8 |
| ATOM | 19086 | NE2 | GLN | M | 1324 | 73.226 | 122.427 | 64.704 | 1.00 | 178.64 | 7 |
| ATOM | 19087 | C | GLN | M | 1324 | 73.114 | 123.068 | 59.888 | 1.00 | 62.73 | 6 |
| ATOM | 19088 | O | GLN | M | 1324 | 72.434 | 124.092 | 59.869 | 1.00 | 63.05 | 8 |
| ATOM | 19089 | N | PRO | M | 1325 | 73.149 | 122.232 | 58.839 | 1.00 | 82.54 | 7 |
| ATOM | 19090 | CD | PRO | M | 1325 | 74.250 | 121.338 | 58.436 | 1.00 | 101.31 | 6 |
| ATOM | 19091 | CA | PRO | M | 1325 | 72.321 | 122.552 | 57.677 | 1.00 | 82.86 | 6 |
| ATOM | 19092 | CB | PRO | M | 1325 | 73.278 | 122.346 | 56.527 | 1.00 | 101.32 | 6 |
| ATOM | 19093 | CG | PRO | M | 1325 | 73.967 | 121.108 | 56.961 | 1.00 | 101.85 | 6 |
| ATOM | 19094 | C | PRO | M | 1325 | 71.215 | 121.517 | 57.676 | 1.00 | 83.19 | 6 |
| ATOM | 19095 | O | PRO | M | 1325 | 70.057 | 121.811 | 57.426 | 1.00 | 83.28 | 8 |
| ATOM | 19096 | N | ALA | M | 1326 | 71.622 | 120.295 | 57.985 | 1.00 | 100.14 | 7 |
| ATOM | 19097 | CA | ALA | M | 1326 | 70.770 | 119.123 | 58.043 | 1.00 | 101.85 | 6 |
| ATOM | 19098 | CB | ALA | M | 1326 | 70.983 | 118.401 | 59.362 | 1.00 | 128.51 | 6 |
| ATOM | 19099 | C | ALA | M | 1326 | 69.292 | 119.303 | 57.812 | 1.00 | 103.03 | 6 |
| ATOM | 19100 | O | ALA | M | 1326 | 68.629 | 120.120 | 58.448 | 1.00 | 103.18 | 8 |
| ATOM | 19101 | N | ALA | M | 1327 | 68.785 | 118.521 | 56.876 | 1.00 | 208.87 | 7 |
| ATOM | 19102 | CA | ALA | M | 1327 | 67.372 | 118.511 | 56.577 | 1.00 | 208.87 | 6 |
| ATOM | 19103 | CB | ALA | M | 1327 | 67.147 | 118.647 | 55.083 | 1.00 | 151.94 | 6 |
| ATOM | 19104 | C | ALA | M | 1327 | 67.030 | 117.106 | 57.057 | 1.00 | 208.87 | 6 |
| ATOM | 19105 | O | ALA | M | 1327 | 65.944 | 116.578 | 56.801 | 1.00 | 208.87 | 8 |
| ATOM | 19106 | N | ARG | M | 1328 | 68.004 | 116.534 | 57.770 | 1.00 | 208.87 | 7 |
| ATOM | 19107 | CA | ARG | M | 1328 | 67.966 | 115.193 | 58.344 | 1.00 | 208.87 | 6 |
| ATOM | 19108 | CB | ARG | M | 1328 | 66.517 | 114.775 | 58.675 | 1.00 | 206.75 | 6 |
| ATOM | 19109 | CG | ARG | M | 1328 | 65.971 | 113.606 | 57.868 | 1.00 | 207.80 | 6 |
| ATOM | 19110 | CD | ARG | M | 1328 | 64.507 | 113.333 | 58.169 | 1.00 | 208.71 | 6 |
| ATOM | 19111 | NE | ARG | M | 1328 | 63.624 | 114.335 | 57.579 | 1.00 | 208.87 | 7 |
| ATOM | 19112 | CZ | ARG | M | 1328 | 63.399 | 115.539 | 58.093 | 1.00 | 208.87 | 6 |
| ATOM | 19113 | NH1 | ARG | M | 1328 | 63.991 | 115.908 | 59.221 | 1.00 | 208.87 | 7 |
| ATOM | 19114 | NH2 | ARG | M | 1328 | 62.577 | 116.378 | 57.477 | 1.00 | 208.87 | 7 |
| ATOM | 19115 | C | ARG | M | 1328 | 68.634 | 114.196 | 57.390 | 1.00 | 208.87 | 6 |
| ATOM | 19116 | O | ARG | M | 1328 | 68.627 | 112.988 | 57.633 | 1.00 | 208.87 | 8 |
| ATOM | 19117 | N | GLY | M | 1329 | 69.233 | 114.707 | 56.314 | 1.00 | 71.92 | 7 |
| ATOM | 19118 | CA | GLY | M | 1329 | 69.879 | 113.821 | 55.362 | 1.00 | 69.86 | 6 |
| ATOM | 19119 | C | GLY | M | 1329 | 71.369 | 113.721 | 55.597 | 1.00 | 68.76 | 6 |
| ATOM | 19120 | O | GLY | M | 1329 | 72.151 | 114.215 | 54.790 | 1.00 | 69.45 | 8 |
| ATOM | 19121 | N | ALA | M | 1330 | 71.753 | 113.067 | 56.694 | 1.00 | 68.05 | 7 |
| ATOM | 19122 | CA | ALA | M | 1330 | 73.160 | 112.908 | 57.082 | 1.00 | 66.72 | 6 |
| ATOM | 19123 | CB | ALA | M | 1330 | 73.332 | 111.658 | 57.938 | 1.00 | 112.89 | 6 |
| ATOM | 19124 | C | ALA | M | 1330 | 74.121 | 112.875 | 55.903 | 1.00 | 65.68 | 6 |
| ATOM | 19125 | O | ALA | M | 1330 | 74.193 | 111.891 | 55.169 | 1.00 | 65.37 | 8 |
| ATOM | 19126 | N | ALA | M | 1331 | 74.856 | 113.972 | 55.738 | 1.00 | 149.64 | 7 |
| ATOM | 19127 | CA | ALA | M | 1331 | 75.817 | 114.115 | 54.652 | 1.00 | 149.47 | 6 |
| ATOM | 19128 | CB | ALA | M | 1331 | 76.919 | 115.112 | 55.043 | 1.00 | 100.75 | 6 |
| ATOM | 19129 | C | ALA | M | 1331 | 76.433 | 112.778 | 54.283 | 1.00 | 149.01 | 6 |
| ATOM | 19130 | O | ALA | M | 1331 | 76.557 | 111.884 | 55.125 | 1.00 | 149.88 | 8 |
| ATOM | 19131 | N | ALA | M | 1332 | 76.803 | 112.652 | 53.013 | 1.00 | 120.55 | 7 |
| ATOM | 19132 | CA | ALA | M | 1332 | 77.414 | 111.440 | 52.493 | 1.00 | 118.71 | 6 |
| ATOM | 19133 | CB | ALA | M | 1332 | 78.474 | 111.813 | 51.450 | 1.00 | 61.10 | 6 |
| ATOM | 19134 | C | ALA | M | 1332 | 78.041 | 110.617 | 53.623 | 1.00 | 117.29 | 6 |
| ATOM | 19135 | O | ALA | M | 1332 | 79.100 | 110.971 | 54.127 | 1.00 | 117.59 | 8 |
| ATOM | 19136 | N | PRO | M | 1333 | 77.372 | 109.526 | 54.057 | 1.00 | 83.42 | 7 |
| ATOM | 19137 | CD | PRO | M | 1333 | 76.088 | 109.004 | 53.555 | 1.00 | 136.34 | 6 |
| ATOM | 19138 | CA | PRO | M | 1333 | 77.881 | 108.669 | 55.125 | 1.00 | 80.62 | 6 |
| ATOM | 19139 | CB | PRO | M | 1333 | 77.118 | 107.377 | 54.904 | 1.00 | 133.70 | 6 |
| ATOM | 19140 | CG | PRO | M | 1333 | 75.771 | 107.892 | 54.543 | 1.00 | 135.07 | 6 |
| ATOM | 19141 | C | PRO | M | 1333 | 79.389 | 108.492 | 55.028 | 1.00 | 78.89 | 6 |
| ATOM | 19142 | O | PRO | M | 1333 | 80.036 | 108.133 | 56.008 | 1.00 | 79.83 | 8 |
| ATOM | 19143 | N | HIS | M | 1334 | 79.949 | 108.740 | 53.847 | 1.00 | 83.51 | 7 |
| ATOM | 19144 | CA | HIS | M | 1334 | 81.394 | 108.635 | 53.655 | 1.00 | 81.28 | 6 |
| ATOM | 19145 | CB | HIS | M | 1334 | 81.758 | 108.784 | 52.181 | 1.00 | 94.91 | 6 |
| ATOM | 19146 | CG | HIS | M | 1334 | 81.530 | 107.546 | 51.378 | 1.00 | 95.11 | 6 |
| ATOM | 19147 | CD2 | HIS | M | 1334 | 80.781 | 107.325 | 50.274 | 1.00 | 95.05 | 6 |
| ATOM | 19148 | ND1 | HIS | M | 1334 | 82.127 | 106.342 | 51.686 | 1.00 | 96.14 | 7 |
| ATOM | 19149 | CE1 | HIS | M | 1334 | 81.754 | 105.432 | 50.804 | 1.00 | 96.08 | 6 |
| ATOM | 19150 | NE2 | HIS | M | 1334 | 80.937 | 106.002 | 49.937 | 1.00 | 96.06 | 7 |
| ATOM | 19151 | C | HIS | M | 1334 | 82.092 | 109.728 | 54.450 | 1.00 | 79.10 | 6 |
| ATOM | 19152 | O | HIS | M | 1334 | 83.249 | 109.584 | 54.847 | 1.00 | 78.74 | 8 |
| ATOM | 19153 | N | GLN | M | 1335 | 81.368 | 110.825 | 54.655 | 1.00 | 78.96 | 7 |
| ATOM | 19154 | CA | GLN | M | 1335 | 81.843 | 111.978 | 55.409 | 1.00 | 77.07 | 6 |
| ATOM | 19155 | CB | GLN | M | 1335 | 80.930 | 113.184 | 55.168 | 1.00 | 62.73 | 6 |
| ATOM | 19156 | CG | GLN | M | 1335 | 81.140 | 114.005 | 53.909 | 1.00 | 63.05 | 6 |
| ATOM | 19157 | CD | GLN | M | 1335 | 79.928 | 114.880 | 53.622 | 1.00 | 63.16 | 6 |
| ATOM | 19158 | OE1 | GLN | M | 1335 | 78.992 | 114.451 | 52.948 | 1.00 | 62.30 | 8 |
| ATOM | 19159 | NE2 | GLN | M | 1335 | 79.924 | 116.097 | 54.162 | 1.00 | 65.06 | 7 |
| ATOM | 19160 | C | GLN | M | 1335 | 81.796 | 111.640 | 56.899 | 1.00 | 76.06 | 6 |
| ATOM | 19161 | O | GLN | M | 1335 | 82.764 | 111.854 | 57.638 | 1.00 | 77.25 | 8 |
| ATOM | 19162 | N | LEU | M | 1336 | 80.648 | 111.136 | 57.341 | 1.00 | 51.91 | 7 |
| ATOM | 19163 | CA | LEU | M | 1336 | 80.486 | 110.773 | 58.730 | 1.00 | 48.96 | 6 |
| ATOM | 19164 | CB | LEU | M | 1336 | 79.127 | 110.166 | 58.951 | 1.00 | 14.76 | 6 |
| ATOM | 19165 | CG | LEU | M | 1336 | 79.055 | 109.563 | 60.345 | 1.00 | 13.94 | 6 |
| ATOM | 19166 | CD1 | LEU | M | 1336 | 78.413 | 110.569 | 61.307 | 1.00 | 13.87 | 6 |
| ATOM | 19167 | CD2 | LEU | M | 1336 | 78.275 | 108.255 | 60.283 | 1.00 | 14.47 | 6 |
| ATOM | 19168 | C | LEU | M | 1336 | 81.560 | 109.765 | 59.109 | 1.00 | 48.58 | 6 |
| ATOM | 19169 | O | LEU | M | 1336 | 82.291 | 109.967 | 60.079 | 1.00 | 49.17 | 8 |
| ATOM | 19170 | N | LEU | M | 1337 | 81.652 | 108.673 | 58.355 | 1.00 | 42.86 | 7 |
| ATOM | 19171 | CA | LEU | M | 1337 | 82.684 | 107.685 | 58.626 | 1.00 | 42.77 | 6 |
| ATOM | 19172 | CB | LEU | M | 1337 | 82.784 | 106.660 | 57.492 | 1.00 | 34.32 | 6 |
| ATOM | 19173 | CG | LEU | M | 1337 | 84.063 | 105.802 | 57.441 | 1.00 | 32.83 | 6 |
| ATOM | 19174 | CD1 | LEU | M | 1337 | 84.467 | 105.328 | 58.819 | 1.00 | 32.74 | 6 |
| ATOM | 19175 | CD2 | LEU | M | 1337 | 83.841 | 104.608 | 56.540 | 1.00 | 31.78 | 6 |
| ATOM | 19176 | C | LEU | M | 1337 | 84.003 | 108.428 | 58.746 | 1.00 | 43.69 | 6 |
| ATOM | 19177 | O | LEU | M | 1337 | 84.677 | 108.340 | 59.770 | 1.00 | 44.59 | 8 |
| ATOM | 19178 | N | GLU | M | 1338 | 84.353 | 109.177 | 57.698 | 1.00 | 55.65 | 7 |
| ATOM | 19179 | CA | GLU | M | 1338 | 85.604 | 109.939 | 57.664 | 1.00 | 57.05 | 6 |
| ATOM | 19180 | CB | GLU | M | 1338 | 85.593 | 110.974 | 56.539 | 1.00 | 130.02 | 6 |
| ATOM | 19181 | CG | GLU | M | 1338 | 86.906 | 111.736 | 56.419 | 1.00 | 134.03 | 6 |
| ATOM | 19182 | CD | GLU | M | 1338 | 88.096 | 110.812 | 56.188 | 1.00 | 135.60 | 6 |
| ATOM | 19183 | OE1 | GLU | M | 1338 | 88.174 | 110.196 | 55.102 | 1.00 | 135.05 | 8 |
| ATOM | 19184 | OE2 | GLU | M | 1338 | 88.950 | 110.696 | 57.095 | 1.00 | 137.56 | 8 |
| ATOM | 19185 | C | GLU | M | 1338 | 85.882 | 110.645 | 58.969 | 1.00 | 57.54 | 6 |
| ATOM | 19186 | O | GLU | M | 1338 | 87.023 | 110.721 | 59.414 | 1.00 | 57.85 | 8 |
| ATOM | 19187 | N | ALA | M | 1339 | 84.828 | 111.164 | 59.576 | 1.00 | 61.79 | 7 |
| ATOM | 19188 | CA | ALA | M | 1339 | 84.964 | 111.852 | 60.835 | 1.00 | 62.44 | 6 |
| ATOM | 19189 | CB | ALA | M | 1339 | 84.783 | 113.331 | 60.625 | 1.00 | 63.58 | 6 |
| ATOM | 19190 | C | ALA | M | 1339 | 83.947 | 111.316 | 61.829 | 1.00 | 63.55 | 6 |
| ATOM | 19191 | O | ALA | M | 1339 | 82.907 | 111.924 | 62.067 | 1.00 | 64.92 | 8 |
| ATOM | 19192 | N | LYS | M | 1340 | 84.264 | 110.147 | 62.375 | 1.00 | 20.48 | 7 |
| ATOM | 19193 | CA | LYS | M | 1340 | 83.463 | 109.443 | 63.376 | 1.00 | 20.92 | 6 |
| ATOM | 19194 | CB | LYS | M | 1340 | 81.979 | 109.797 | 63.266 | 1.00 | 116.40 | 6 |
| ATOM | 19195 | CG | LYS | M | 1340 | 81.276 | 109.687 | 64.602 | 1.00 | 120.37 | 6 |
| ATOM | 19196 | CD | LYS | M | 1340 | 82.096 | 110.397 | 65.683 | 1.00 | 122.67 | 6 |
| ATOM | 19197 | CE | LYS | M | 1340 | 81.708 | 109.950 | 67.087 | 1.00 | 124.05 | 6 |
| ATOM | 19198 | NZ | LYS | M | 1340 | 82.634 | 110.497 | 68.118 | 1.00 | 125.09 | 7 |
| ATOM | 19199 | C | LYS | M | 1340 | 83.653 | 107.927 | 63.281 | 1.00 | 19.83 | 6 |
| ATOM | 19200 | O | LYS | M | 1340 | 83.144 | 107.176 | 64.105 | 1.00 | 19.94 | 8 |
| ATOM | 19201 | N | GLY | M | 1341 | 84.391 | 107.483 | 62.267 | 1.00 | 53.79 | 7 |
| ATOM | 19202 | CA | GLY | M | 1341 | 84.667 | 106.063 | 62.094 | 1.00 | 54.10 | 6 |
| ATOM | 19203 | C | GLY | M | 1341 | 83.566 | 105.182 | 61.526 | 1.00 | 53.76 | 6 |
| ATOM | 19204 | O | GLY | M | 1341 | 82.575 | 105.680 | 60.999 | 1.00 | 54.84 | 8 |
| ATOM | 19205 | N | PRO | M | 1342 | 83.742 | 103.852 | 61.577 | 1.00 | 39.93 | 7 |
| ATOM | 19206 | CD | PRO | M | 1342 | 85.023 | 103.183 | 61.858 | 1.00 | 71.77 | 6 |
| ATOM | 19207 | CA | PRO | M | 1342 | 82.769 | 102.878 | 61.077 | 1.00 | 39.54 | 6 |
| ATOM | 19208 | CB | PRO | M | 1342 | 83.639 | 101.681 | 60.744 | 1.00 | 71.12 | 6 |
| ATOM | 19209 | CG | PRO | M | 1342 | 84.627 | 101.720 | 61.845 | 1.00 | 71.46 | 6 |
| ATOM | 19210 | C | PRO | M | 1342 | 81.763 | 102.542 | 62.164 | 1.00 | 39.38 | 6 |
| ATOM | 19211 | O | PRO | M | 1342 | 80.649 | 102.098 | 61.888 | 1.00 | 39.75 | 8 |
| ATOM | 19212 | N | GLU | M | 1343 | 82.180 | 102.742 | 63.407 | 1.00 | 110.36 | 7 |
| ATOM | 19213 | CA | GLU | M | 1343 | 81.324 | 102.472 | 64.548 | 1.00 | 111.07 | 6 |
| ATOM | 19214 | CB | GLU | M | 1343 | 82.000 | 102.939 | 65.843 | 1.00 | 116.77 | 6 |
| ATOM | 19215 | CG | GLU | M | 1343 | 83.474 | 102.565 | 65.949 | 1.00 | 120.21 | 6 |
| ATOM | 19216 | CD | GLU | M | 1343 | 84.384 | 103.577 | 65.271 | 1.00 | 122.14 | 6 |
| ATOM | 19217 | OE1 | GLU | M | 1343 | 83.996 | 104.110 | 64.212 | 1.00 | 123.85 | 8 |
| ATOM | 19218 | OE2 | GLU | M | 1343 | 85.490 | 103.837 | 65.792 | 1.00 | 122.14 | 8 |
| ATOM | 19219 | C | GLU | M | 1343 | 80.026 | 103.239 | 64.332 | 1.00 | 110.60 | 6 |
| ATOM | 19220 | O | GLU | M | 1343 | 78.933 | 102.675 | 64.414 | 1.00 | 111.23 | 8 |
| ATOM | 19221 | N | ALA | M | 1344 | 80.159 | 104.530 | 64.042 | 1.00 | 60.41 | 7 |
| ATOM | 19222 | CA | ALA | M | 1344 | 79.005 | 105.384 | 63.810 | 1.00 | 56.94 | 6 |
| ATOM | 19223 | CB | ALA | M | 1344 | 79.449 | 106.816 | 63.604 | 1.00 | 110.71 | 6 |
| ATOM | 19224 | C | ALA | M | 1344 | 78.283 | 104.887 | 62.584 | 1.00 | 54.06 | 6 |
| ATOM | 19225 | O | ALA | M | 1344 | 77.228 | 104.275 | 62.677 | 1.00 | 54.80 | 8 |
| ATOM | 19226 | N | VAL | M | 1345 | 78.871 | 105.151 | 61.432 | 1.00 | 39.51 | 7 |
| ATOM | 19227 | CA | VAL | M | 1345 | 78.291 | 104.730 | 60.175 | 1.00 | 37.26 | 6 |
| ATOM | 19228 | CB | VAL | M | 1345 | 79.381 | 104.502 | 59.133 | 1.00 | 51.64 | 6 |
| ATOM | 19229 | CG1 | VAL | M | 1345 | 78.787 | 103.817 | 57.890 | 1.00 | 50.18 | 6 |
| ATOM | 19230 | CG2 | VAL | M | 1345 | 80.011 | 105.846 | 58.772 | 1.00 | 52.02 | 6 |
| ATOM | 19231 | C | VAL | M | 1345 | 77.426 | 103.473 | 60.267 | 1.00 | 35.69 | 6 |
| ATOM | 19232 | O | VAL | M | 1345 | 76.256 | 103.496 | 59.885 | 1.00 | 36.80 | 8 |
| ATOM | 19233 | N | GLU | M | 1346 | 77.993 | 102.374 | 60.753 | 1.00 | 24.68 | 7 |
| ATOM | 19234 | CA | GLU | M | 1346 | 77.208 | 101.159 | 60.871 | 1.00 | 21.54 | 6 |
| ATOM | 19235 | CB | GLU | M | 1346 | 77.924 | 100.115 | 61.717 | 1.00 | 55.67 | 6 |
| ATOM | 19236 | CG | GLU | M | 1346 | 79.060 | 99.391 | 61.060 | 1.00 | 59.34 | 6 |
| ATOM | 19237 | CD | GLU | M | 1346 | 79.650 | 98.355 | 61.992 | 1.00 | 61.81 | 6 |
| ATOM | 19238 | OE1 | GLU | M | 1346 | 78.917 | 97.422 | 62.386 | 1.00 | 62.82 | 8 |
| ATOM | 19239 | OE2 | GLU | M | 1346 | 80.842 | 98.482 | 62.344 | 1.00 | 62.58 | 8 |
| ATOM | 19240 | C | GLU | M | 1346 | 75.917 | 101.517 | 61.574 | 1.00 | 17.78 | 6 |
| ATOM | 19241 | O | GLU | M | 1346 | 74.839 | 101.532 | 60.972 | 1.00 | 15.48 | 8 |
| ATOM | 19242 | N | ARG | M | 1347 | 76.046 | 101.824 | 62.858 | 1.00 | 13.87 | 7 |
| ATOM | 19243 | CA | ARG | M | 1347 | 74.891 | 102.148 | 63.655 | 1.00 | 13.87 | 6 |
| ATOM | 19244 | CB | ARG | M | 1347 | 75.305 | 102.444 | 65.095 | 1.00 | 78.35 | 6 |
| ATOM | 19245 | CG | ARG | M | 1347 | 74.182 | 102.202 | 66.105 | 1.00 | 84.10 | 6 |
| ATOM | 19246 | CD | ARG | M | 1347 | 73.758 | 100.732 | 66.154 | 1.00 | 87.61 | 6 |
| ATOM | 19247 | NE | ARG | M | 1347 | 72.398 | 100.580 | 66.668 | 1.00 | 89.27 | 7 |
| ATOM | 19248 | CZ | ARG | M | 1347 | 71.451 | 99.858 | 66.072 | 1.00 | 90.63 | 6 |
| ATOM | 19249 | NH1 | ARG | M | 1347 | 71.717 | 99.214 | 64.943 | 1.00 | 88.28 | 7 |
| ATOM | 19250 | NH2 | ARG | M | 1347 | 70.228 | 99.800 | 66.588 | 1.00 | 92.19 | 7 |
| ATOM | 19251 | C | ARG | M | 1347 | 74.113 | 103.308 | 63.057 | 1.00 | 13.87 | 6 |
| ATOM | 19252 | O | ARG | M | 1347 | 72.986 | 103.564 | 63.463 | 1.00 | 13.87 | 8 |
| ATOM | 19253 | N | TYR | M | 1348 | 74.698 | 104.020 | 62.096 | 1.00 | 22.17 | 7 |
| ATOM | 19254 | CA | TYR | M | 1348 | 73.956 | 105.106 | 61.477 | 1.00 | 20.27 | 6 |
| ATOM | 19255 | CB | TYR | M | 1348 | 74.811 | 106.233 | 60.919 | 1.00 | 20.01 | 6 |
| ATOM | 19256 | CG | TYR | M | 1348 | 73.909 | 107.186 | 60.163 | 1.00 | 18.85 | 6 |
| ATOM | 19257 | CD1 | TYR | M | 1348 | 72.764 | 107.686 | 60.764 | 1.00 | 19.10 | 6 |
| ATOM | 19258 | CE1 | TYR | M | 1348 | 71.867 | 108.462 | 60.084 | 1.00 | 18.16 | 6 |
| ATOM | 19259 | CD2 | TYR | M | 1348 | 74.128 | 107.504 | 58.842 | 1.00 | 19.50 | 6 |
| ATOM | 19260 | CE2 | TYR | M | 1348 | 73.218 | 108.295 | 58.141 | 1.00 | 19.53 | 6 |
| ATOM | 19261 | CZ | TYR | M | 1348 | 72.093 | 108.767 | 58.785 | 1.00 | 19.20 | 6 |
| ATOM | 19262 | OH | TYR | M | 1348 | 71.191 | 109.582 | 58.156 | 1.00 | 22.06 | 8 |
| ATOM | 19263 | C | TYR | M | 1348 | 73.203 | 104.562 | 60.311 | 1.00 | 21.43 | 6 |
| ATOM | 19264 | O | TYR | M | 1348 | 72.127 | 105.049 | 59.986 | 1.00 | 21.47 | 8 |
| ATOM | 19265 | N | LEU | M | 1349 | 73.799 | 103.576 | 59.652 | 1.00 | 67.99 | 7 |
| ATOM | 19266 | CA | LEU | M | 1349 | 73.186 | 102.943 | 58.498 | 1.00 | 69.05 | 6 |
| ATOM | 19267 | CB | LEU | M | 1349 | 74.197 | 102.011 | 57.813 | 1.00 | 27.21 | 6 |
| ATOM | 19268 | CG | LEU | M | 1349 | 74.654 | 102.470 | 56.428 | 1.00 | 24.57 | 6 |
| ATOM | 19269 | CD1 | LEU | M | 1349 | 74.591 | 103.979 | 56.348 | 1.00 | 23.50 | 6 |
| ATOM | 19270 | CD2 | LEU | M | 1349 | 76.052 | 101.971 | 56.147 | 1.00 | 23.96 | 6 |
| ATOM | 19271 | C | LEU | M | 1349 | 71.967 | 102.170 | 58.964 | 1.00 | 70.22 | 6 |
| ATOM | 19272 | O | LEU | M | 1349 | 70.888 | 102.291 | 58.386 | 1.00 | 71.16 | 8 |
| ATOM | 19273 | N | VAL | M | 1350 | 72.139 | 101.396 | 60.029 | 1.00 | 38.88 | 7 |
| ATOM | 19274 | CA | VAL | M | 1350 | 71.038 | 100.613 | 60.556 | 1.00 | 39.64 | 6 |
| ATOM | 19275 | CB | VAL | M | 1350 | 71.478 | 99.755 | 61.752 | 1.00 | 82.77 | 6 |
| ATOM | 19276 | CG1 | VAL | M | 1350 | 70.286 | 99.038 | 62.333 | 1.00 | 82.95 | 6 |
| ATOM | 19277 | CG2 | VAL | M | 1350 | 72.527 | 98.749 | 61.312 | 1.00 | 84.45 | 6 |
| ATOM | 19278 | C | VAL | M | 1350 | 69.887 | 101.509 | 60.993 | 1.00 | 39.38 | 6 |
| ATOM | 19279 | O | VAL | M | 1350 | 68.855 | 101.580 | 60.327 | 1.00 | 39.26 | 8 |
| ATOM | 19280 | N | ASP | M | 1351 | 70.064 | 102.205 | 62.108 | 1.00 | 32.57 | 7 |
| ATOM | 19281 | CA | ASP | M | 1351 | 69.002 | 103.055 | 62.606 | 1.00 | 33.77 | 6 |
| ATOM | 19282 | CB | ASP | M | 1351 | 69.514 | 104.009 | 63.693 | 1.00 | 53.66 | 6 |
| ATOM | 19283 | CG | ASP | M | 1351 | 69.545 | 103.363 | 65.083 | 1.00 | 55.50 | 6 |
| ATOM | 19284 | OD1 | ASP | M | 1351 | 68.500 | 102.857 | 65.543 | 1.00 | 55.66 | 8 |
| ATOM | 19285 | OD2 | ASP | M | 1351 | 70.616 | 103.367 | 65.725 | 1.00 | 57.05 | 8 |
| ATOM | 19286 | C | ASP | M | 1351 | 68.359 | 103.837 | 61.479 | 1.00 | 33.69 | 6 |
| ATOM | 19287 | O | ASP | M | 1351 | 67.153 | 103.756 | 61.281 | 1.00 | 33.24 | 8 |
| ATOM | 19288 | N | GLU | M | 1352 | 69.165 | 104.554 | 60.709 | 1.00 | 31.50 | 7 |
| ATOM | 19289 | CA | GLU | M | 1352 | 68.637 | 105.378 | 59.632 | 1.00 | 32.99 | 6 |
| ATOM | 19290 | CB | GLU | M | 1352 | 69.776 | 106.138 | 58.959 | 1.00 | 45.71 | 6 |
| ATOM | 19291 | CG | GLU | M | 1352 | 69.323 | 107.291 | 58.087 | 1.00 | 47.39 | 6 |
| ATOM | 19292 | CD | GLU | M | 1352 | 68.407 | 108.257 | 58.815 | 1.00 | 47.45 | 6 |
| ATOM | 19293 | OE1 | GLU | M | 1352 | 67.176 | 108.043 | 58.776 | 1.00 | 46.97 | 8 |
| ATOM | 19294 | OE2 | GLU | M | 1352 | 68.922 | 109.219 | 59.427 | 1.00 | 47.46 | 8 |
| ATOM | 19295 | C | GLU | M | 1352 | 67.800 | 104.647 | 58.583 | 1.00 | 34.27 | 6 |
| ATOM | 19296 | O | GLU | M | 1352 | 66.940 | 105.260 | 57.939 | 1.00 | 34.81 | 8 |
| ATOM | 19297 | N | ILE | M | 1353 | 68.034 | 103.351 | 58.399 | 1.00 | 46.14 | 7 |
| ATOM | 19298 | CA | ILE | M | 1353 | 67.248 | 102.606 | 57.418 | 1.00 | 45.23 | 6 |
| ATOM | 19299 | CB | ILE | M | 1353 | 67.895 | 101.275 | 57.061 | 1.00 | 13.96 | 6 |
| ATOM | 19300 | CG2 | ILE | M | 1353 | 66.917 | 100.436 | 56.253 | 1.00 | 13.87 | 6 |
| ATOM | 19301 | CG1 | ILE | M | 1353 | 69.203 | 101.526 | 56.304 | 1.00 | 13.87 | 6 |
| ATOM | 19302 | CD1 | ILE | M | 1353 | 69.996 | 100.277 | 56.008 | 1.00 | 13.87 | 6 |
| ATOM | 19303 | C | ILE | M | 1353 | 65.855 | 102.328 | 57.958 | 1.00 | 46.27 | 6 |
| ATOM | 19304 | O | ILE | M | 1353 | 64.856 | 102.645 | 57.310 | 1.00 | 46.46 | 8 |
| ATOM | 19305 | N | GLN | M | 1354 | 65.796 | 101.724 | 59.143 | 1.00 | 34.00 | 7 |
| ATOM | 19306 | CA | GLN | M | 1354 | 64.521 | 101.426 | 59.785 | 1.00 | 33.64 | 6 |
| ATOM | 19307 | CB | GLN | M | 1354 | 64.715 | 101.244 | 61.294 | 1.00 | 20.04 | 6 |
| ATOM | 19308 | CG | GLN | M | 1354 | 65.309 | 99.902 | 61.696 | 1.00 | 16.27 | 6 |
| ATOM | 19309 | CD | GLN | M | 1354 | 64.316 | 98.774 | 61.570 | 1.00 | 13.87 | 6 |
| ATOM | 19310 | OE1 | GLN | M | 1354 | 63.361 | 98.689 | 62.325 | 1.00 | 13.87 | 8 |
| ATOM | 19311 | NE2 | GLN | M | 1354 | 64.533 | 97.909 | 60.605 | 1.00 | 14.26 | 7 |
| ATOM | 19312 | C | GLN | M | 1354 | 63.569 | 102.586 | 59.525 | 1.00 | 34.96 | 6 |
| ATOM | 19313 | O | GLN | M | 1354 | 62.573 | 102.430 | 58.821 | 1.00 | 34.64 | 8 |
| ATOM | 19314 | N | LYS | M | 1355 | 63.912 | 103.751 | 60.070 | 1.00 | 49.00 | 7 |
| ATOM | 19315 | CA | LYS | M | 1355 | 63.110 | 104.961 | 59.930 | 1.00 | 50.54 | 6 |
| ATOM | 19316 | CB | LYS | M | 1355 | 64.015 | 106.187 | 59.779 | 1.00 | 144.26 | 6 |
| ATOM | 19317 | CG | LYS | M | 1355 | 64.802 | 106.526 | 61.035 | 1.00 | 148.98 | 6 |
| ATOM | 19318 | CD | LYS | M | 1355 | 65.414 | 107.917 | 60.955 | 1.00 | 151.56 | 6 |
| ATOM | 19319 | CE | LYS | M | 1355 | 66.185 | 108.261 | 62.222 | 1.00 | 152.92 | 6 |
| ATOM | 19320 | NZ | LYS | M | 1355 | 66.770 | 109.630 | 62.165 | 1.00 | 153.96 | 7 |
| ATOM | 19321 | C | LYS | M | 1355 | 62.110 | 104.926 | 58.787 | 1.00 | 49.98 | 6 |
| ATOM | 19322 | O | LYS | M | 1355 | 60.924 | 105.181 | 59.003 | 1.00 | 50.04 | 8 |
| ATOM | 19323 | N | VAL | M | 1356 | 62.578 | 104.601 | 57.579 | 1.00 | 36.25 | 7 |
| ATOM | 19324 | CA | VAL | M | 1356 | 61.696 | 104.557 | 56.411 | 1.00 | 35.59 | 6 |
| ATOM | 19325 | CB | VAL | M | 1356 | 62.459 | 104.280 | 55.111 | 1.00 | 99.73 | 6 |
| ATOM | 19326 | CG1 | VAL | M | 1356 | 61.496 | 104.309 | 53.940 | 1.00 | 100.72 | 6 |
| ATOM | 19327 | CG2 | VAL | M | 1356 | 63.540 | 105.328 | 54.913 | 1.00 | 101.97 | 6 |
| ATOM | 19328 | C | VAL | M | 1356 | 60.666 | 103.478 | 56.609 | 1.00 | 34.12 | 6 |
| ATOM | 19329 | O | VAL | M | 1356 | 59.469 | 103.711 | 56.450 | 1.00 | 33.45 | 8 |
| ATOM | 19330 | N | TYR | M | 1357 | 61.124 | 102.285 | 56.944 | 1.00 | 26.83 | 7 |
| ATOM | 19331 | CA | TYR | M | 1357 | 60.176 | 101.229 | 57.196 | 1.00 | 27.60 | 6 |
| ATOM | 19332 | CB | TYR | M | 1357 | 60.897 | 99.928 | 57.519 | 1.00 | 32.03 | 6 |
| ATOM | 19333 | CG | TYR | M | 1357 | 61.531 | 99.257 | 56.323 | 1.00 | 29.83 | 6 |
| ATOM | 19334 | CD1 | TYR | M | 1357 | 62.316 | 98.125 | 56.483 | 1.00 | 29.38 | 6 |
| ATOM | 19335 | CE1 | TYR | M | 1357 | 62.900 | 97.498 | 55.406 | 1.00 | 28.03 | 6 |
| ATOM | 19336 | CD2 | TYR | M | 1357 | 61.344 | 99.743 | 55.036 | 1.00 | 29.13 | 6 |
| ATOM | 19337 | CE2 | TYR | M | 1357 | 61.922 | 99.117 | 53.953 | 1.00 | 28.27 | 6 |
| ATOM | 19338 | CZ | TYR | M | 1357 | 62.699 | 97.999 | 54.151 | 1.00 | 28.10 | 6 |
| ATOM | 19339 | OH | TYR | M | 1357 | 63.306 | 97.385 | 53.099 | 1.00 | 29.37 | 8 |
| ATOM | 19340 | C | TYR | M | 1357 | 59.345 | 101.689 | 58.386 | 1.00 | 29.83 | 6 |
| ATOM | 19341 | O | TYR | M | 1357 | 58.179 | 102.027 | 58.233 | 1.00 | 30.32 | 8 |
| ATOM | 19342 | N | ARG | M | 1358 | 59.964 | 101.727 | 59.564 | 1.00 | 41.68 | 7 |
| ATOM | 19343 | CA | ARG | M | 1358 | 59.296 | 102.137 | 60.800 | 1.00 | 43.86 | 6 |
| ATOM | 19344 | CB | ARG | M | 1358 | 60.318 | 102.690 | 61.801 | 1.00 | 40.55 | 6 |
| ATOM | 19345 | CG | ARG | M | 1358 | 61.171 | 101.636 | 62.467 | 1.00 | 39.36 | 6 |
| ATOM | 19346 | CD | ARG | M | 1358 | 62.310 | 102.230 | 63.260 | 1.00 | 37.99 | 6 |
| ATOM | 19347 | NE | ARG | M | 1358 | 63.033 | 101.172 | 63.954 | 1.00 | 39.09 | 7 |
| ATOM | 19348 | CZ | ARG | M | 1358 | 64.135 | 101.356 | 64.673 | 1.00 | 39.01 | 6 |
| ATOM | 19349 | NH1 | ARG | M | 1358 | 64.657 | 102.571 | 64.790 | 1.00 | 39.99 | 7 |
| ATOM | 19350 | NH2 | ARG | M | 1358 | 64.702 | 100.327 | 65.295 | 1.00 | 38.71 | 7 |
| ATOM | 19351 | C | ARG | M | 1358 | 58.206 | 103.172 | 60.601 | 1.00 | 45.79 | 6 |
| ATOM | 19352 | O | ARG | M | 1358 | 57.147 | 103.086 | 61.218 | 1.00 | 45.87 | 8 |
| ATOM | 19353 | N | ALA | M | 1359 | 58.474 | 104.148 | 59.737 | 1.00 | 43.17 | 7 |
| ATOM | 19354 | CA | ALA | M | 1359 | 57.529 | 105.229 | 59.467 | 1.00 | 46.65 | 6 |
| ATOM | 19355 | CB | ALA | M | 1359 | 58.255 | 106.379 | 58.771 | 1.00 | 42.33 | 6 |
| ATOM | 19356 | C | ALA | M | 1359 | 56.296 | 104.809 | 58.656 | 1.00 | 49.24 | 6 |
| ATOM | 19357 | O | ALA | M | 1359 | 55.216 | 105.388 | 58.805 | 1.00 | 50.25 | 8 |
| ATOM | 19358 | N | GLN | M | 1360 | 56.448 | 103.810 | 57.799 | 1.00 | 55.01 | 7 |
| ATOM | 19359 | CA | GLN | M | 1360 | 55.319 | 103.356 | 57.011 | 1.00 | 57.94 | 6 |
| ATOM | 19360 | CB | GLN | M | 1360 | 55.747 | 103.119 | 55.563 | 1.00 | 95.02 | 6 |
| ATOM | 19361 | CG | GLN | M | 1360 | 56.124 | 104.410 | 54.853 | 1.00 | 98.34 | 6 |
| ATOM | 19362 | CD | GLN | M | 1360 | 56.316 | 104.232 | 53.361 | 1.00 | 101.35 | 6 |
| ATOM | 19363 | OE1 | GLN | M | 1360 | 57.251 | 103.570 | 52.917 | 1.00 | 103.64 | 8 |
| ATOM | 19364 | NE2 | GLN | M | 1360 | 55.424 | 104.824 | 52.577 | 1.00 | 103.29 | 7 |
| ATOM | 19365 | C | GLN | M | 1360 | 54.680 | 102.111 | 57.609 | 1.00 | 59.58 | 6 |
| ATOM | 19366 | O | GLN | M | 1360 | 53.803 | 101.505 | 57.002 | 1.00 | 61.56 | 8 |
| ATOM | 19367 | N | GLY | M | 1361 | 55.127 | 101.726 | 58.800 | 1.00 | 66.39 | 7 |
| ATOM | 19368 | CA | GLY | M | 1361 | 54.538 | 100.577 | 59.464 | 1.00 | 67.72 | 6 |
| ATOM | 19369 | C | GLY | M | 1361 | 55.245 | 99.237 | 59.501 | 1.00 | 69.35 | 6 |
| ATOM | 19370 | O | GLY | M | 1361 | 54.779 | 98.345 | 60.197 | 1.00 | 71.07 | 8 |
| ATOM | 19371 | N | VAL | M | 1362 | 56.349 | 99.072 | 58.779 | 1.00 | 69.68 | 7 |
| ATOM | 19372 | CA | VAL | M | 1362 | 57.067 | 97.791 | 58.770 | 1.00 | 70.93 | 6 |
| ATOM | 19373 | CB | VAL | M | 1362 | 58.148 | 97.778 | 57.682 | 1.00 | 114.35 | 6 |
| ATOM | 19374 | CG1 | VAL | M | 1362 | 58.787 | 96.412 | 57.620 | 1.00 | 115.83 | 6 |
| ATOM | 19375 | CG2 | VAL | M | 1362 | 57.540 | 98.151 | 56.338 | 1.00 | 114.45 | 6 |
| ATOM | 19376 | C | VAL | M | 1362 | 57.729 | 97.500 | 60.120 | 1.00 | 71.13 | 6 |
| ATOM | 19377 | O | VAL | M | 1362 | 58.354 | 98.382 | 60.702 | 1.00 | 72.34 | 8 |
| ATOM | 19378 | N | LYS | M | 1363 | 57.629 | 96.268 | 60.616 | 1.00 | 60.31 | 7 |
| ATOM | 19379 | CA | LYS | M | 1363 | 58.216 | 95.991 | 61.916 | 1.00 | 60.45 | 6 |
| ATOM | 19380 | CB | LYS | M | 1363 | 57.134 | 95.531 | 62.895 | 1.00 | 113.54 | 6 |
| ATOM | 19381 | CG | LYS | M | 1363 | 56.250 | 96.656 | 63.402 | 1.00 | 115.84 | 6 |
| ATOM | 19382 | CD | LYS | M | 1363 | 55.180 | 96.153 | 64.365 | 1.00 | 117.22 | 6 |
| ATOM | 19383 | CE | LYS | M | 1363 | 54.220 | 97.276 | 64.757 | 1.00 | 118.04 | 6 |
| ATOM | 19384 | NZ | LYS | M | 1363 | 53.114 | 96.819 | 65.645 | 1.00 | 117.65 | 7 |
| ATOM | 19385 | C | LYS | M | 1363 | 59.399 | 95.054 | 62.022 | 1.00 | 60.10 | 6 |
| ATOM | 19386 | O | LYS | M | 1363 | 59.597 | 94.451 | 63.066 | 1.00 | 61.32 | 8 |
| ATOM | 19387 | N | LEU | M | 1364 | 60.202 | 94.924 | 60.976 | 1.00 | 58.42 | 7 |
| ATOM | 19388 | CA | LEU | M | 1364 | 61.378 | 94.057 | 61.074 | 1.00 | 57.25 | 6 |
| ATOM | 19389 | CB | LEU | M | 1364 | 61.870 | 93.636 | 59.693 | 1.00 | 14.01 | 6 |
| ATOM | 19390 | CG | LEU | M | 1364 | 62.815 | 94.619 | 59.001 | 1.00 | 13.87 | 6 |
| ATOM | 19391 | CD1 | LEU | M | 1364 | 63.224 | 94.088 | 57.667 | 1.00 | 13.87 | 6 |
| ATOM | 19392 | CD2 | LEU | M | 1364 | 62.144 | 95.938 | 58.821 | 1.00 | 15.32 | 6 |
| ATOM | 19393 | C | LEU | M | 1364 | 62.480 | 94.871 | 61.734 | 1.00 | 57.05 | 6 |
| ATOM | 19394 | O | LEU | M | 1364 | 62.358 | 96.086 | 61.864 | 1.00 | 57.32 | 8 |
| ATOM | 19395 | N | HIS | M | 1365 | 63.555 | 94.202 | 62.125 | 1.00 | 55.51 | 7 |
| ATOM | 19396 | CA | HIS | M | 1365 | 64.735 | 94.821 | 62.745 | 1.00 | 54.85 | 6 |
| ATOM | 19397 | CB | HIS | M | 1365 | 64.404 | 96.051 | 63.621 | 1.00 | 89.31 | 6 |
| ATOM | 19398 | CG | HIS | M | 1365 | 65.613 | 96.742 | 64.213 | 1.00 | 89.88 | 6 |
| ATOM | 19399 | CD2 | HIS | M | 1365 | 66.166 | 96.673 | 65.450 | 1.00 | 89.57 | 6 |
| ATOM | 19400 | ND1 | HIS | M | 1365 | 66.375 | 97.652 | 63.512 | 1.00 | 90.33 | 7 |
| ATOM | 19401 | CE1 | HIS | M | 1365 | 67.340 | 98.115 | 64.291 | 1.00 | 89.09 | 6 |
| ATOM | 19402 | NE2 | HIS | M | 1365 | 67.236 | 97.536 | 65.472 | 1.00 | 87.92 | 7 |
| ATOM | 19403 | C | HIS | M | 1365 | 65.263 | 93.741 | 63.630 | 1.00 | 52.35 | 6 |
| ATOM | 19404 | O | HIS | M | 1365 | 64.797 | 92.603 | 63.566 | 1.00 | 51.80 | 8 |
| ATOM | 19405 | N | ASP | M | 1366 | 66.212 | 94.103 | 64.479 | 1.00 | 143.74 | 7 |
| ATOM | 19406 | CA | ASP | M | 1366 | 66.802 | 93.147 | 65.376 | 1.00 | 140.35 | 6 |
| ATOM | 19407 | CB | ASP | M | 1366 | 65.725 | 92.329 | 66.072 | 1.00 | 69.65 | 6 |
| ATOM | 19408 | CG | ASP | M | 1366 | 66.301 | 91.249 | 66.911 | 1.00 | 72.16 | 6 |
| ATOM | 19409 | OD1 | ASP | M | 1366 | 66.912 | 91.583 | 67.940 | 1.00 | 73.07 | 8 |
| ATOM | 19410 | OD2 | ASP | M | 1366 | 66.162 | 90.072 | 66.534 | 1.00 | 73.84 | 8 |
| ATOM | 19411 | C | ASP | M | 1366 | 67.627 | 92.243 | 64.495 | 1.00 | 137.18 | 6 |
| ATOM | 19412 | O | ASP | M | 1366 | 67.154 | 91.191 | 64.062 | 1.00 | 138.07 | 8 |
| ATOM | 19413 | N | LYS | M | 1367 | 68.853 | 92.678 | 64.220 | 1.00 | 72.57 | 7 |
| ATOM | 19414 | CA | LYS | M | 1367 | 69.788 | 91.933 | 63.391 | 1.00 | 65.51 | 6 |
| ATOM | 19415 | CB | LYS | M | 1367 | 69.583 | 90.433 | 63.611 | 1.00 | 31.25 | 6 |
| ATOM | 19416 | CG | LYS | M | 1367 | 69.563 | 90.075 | 65.106 | 1.00 | 29.48 | 6 |
| ATOM | 19417 | CD | LYS | M | 1367 | 69.400 | 88.595 | 65.400 | 1.00 | 28.92 | 6 |
| ATOM | 19418 | CE | LYS | M | 1367 | 68.009 | 88.104 | 65.102 | 1.00 | 30.10 | 6 |
| ATOM | 19419 | NZ | LYS | M | 1367 | 67.901 | 86.644 | 65.347 | 1.00 | 32.28 | 7 |
| ATOM | 19420 | C | LYS | M | 1367 | 69.524 | 92.342 | 61.955 | 1.00 | 61.88 | 6 |
| ATOM | 19421 | O | LYS | M | 1367 | 69.633 | 93.519 | 61.626 | 1.00 | 62.01 | 8 |
| ATOM | 19422 | N | HIS | M | 1368 | 69.155 | 91.385 | 61.117 | 1.00 | 60.17 | 7 |
| ATOM | 19423 | CA | HIS | M | 1368 | 68.869 | 91.643 | 59.713 | 1.00 | 56.41 | 6 |
| ATOM | 19424 | CB | HIS | M | 1368 | 67.349 | 91.771 | 59.515 | 1.00 | 23.36 | 6 |
| ATOM | 19425 | CG | HIS | M | 1368 | 66.606 | 90.516 | 59.873 | 1.00 | 19.79 | 6 |
| ATOM | 19426 | CD2 | HIS | M | 1368 | 65.455 | 90.316 | 60.561 | 1.00 | 18.16 | 6 |
| ATOM | 19427 | ND1 | HIS | M | 1368 | 67.075 | 89.257 | 59.550 | 1.00 | 20.87 | 7 |
| ATOM | 19428 | CE1 | HIS | M | 1368 | 66.249 | 88.338 | 60.026 | 1.00 | 18.52 | 6 |
| ATOM | 19429 | NE2 | HIS | M | 1368 | 65.256 | 88.953 | 60.644 | 1.00 | 18.09 | 7 |
| ATOM | 19430 | C | HIS | M | 1368 | 69.647 | 92.819 | 59.096 | 1.00 | 55.03 | 6 |
| ATOM | 19431 | O | HIS | M | 1368 | 70.727 | 92.587 | 58.546 | 1.00 | 57.26 | 8 |
| ATOM | 19432 | N | ILE | M | 1369 | 69.150 | 94.059 | 59.182 | 1.00 | 25.30 | 7 |
| ATOM | 19433 | CA | ILE | M | 1369 | 69.879 | 95.200 | 58.593 | 1.00 | 22.61 | 6 |
| ATOM | 19434 | CB | ILE | M | 1369 | 69.307 | 96.586 | 58.992 | 1.00 | 14.91 | 6 |
| ATOM | 19435 | CG2 | ILE | M | 1369 | 69.693 | 97.601 | 57.943 | 1.00 | 15.38 | 6 |
| ATOM | 19436 | CG1 | ILE | M | 1369 | 67.786 | 96.584 | 58.998 | 1.00 | 13.87 | 6 |
| ATOM | 19437 | CD1 | ILE | M | 1369 | 67.189 | 97.959 | 59.235 | 1.00 | 13.87 | 6 |
| ATOM | 19438 | C | ILE | M | 1369 | 71.342 | 95.203 | 59.022 | 1.00 | 22.50 | 6 |
| ATOM | 19439 | O | ILE | M | 1369 | 72.222 | 95.502 | 58.223 | 1.00 | 22.96 | 8 |
| ATOM | 19440 | N | GLU | M | 1370 | 71.598 | 94.868 | 60.285 | 1.00 | 26.82 | 7 |
| ATOM | 19441 | CA | GLU | M | 1370 | 72.961 | 94.833 | 60.803 | 1.00 | 25.65 | 6 |
| ATOM | 19442 | CB | GLU | M | 1370 | 72.955 | 94.549 | 62.318 | 1.00 | 26.07 | 6 |
| ATOM | 19443 | CG | GLU | M | 1370 | 71.953 | 95.450 | 63.083 | 1.00 | 28.74 | 6 |
| ATOM | 19444 | CD | GLU | M | 1370 | 72.238 | 95.651 | 64.585 | 1.00 | 29.30 | 6 |
| ATOM | 19445 | OE1 | GLU | M | 1370 | 72.470 | 94.660 | 65.307 | 1.00 | 26.57 | 8 |
| ATOM | 19446 | OE2 | GLU | M | 1370 | 72.200 | 96.816 | 65.053 | 1.00 | 30.31 | 8 |
| ATOM | 19447 | C | GLU | M | 1370 | 73.772 | 93.798 | 60.022 | 1.00 | 24.37 | 6 |
| ATOM | 19448 | O | GLU | M | 1370 | 74.768 | 94.158 | 59.407 | 1.00 | 24.40 | 8 |
| ATOM | 19449 | N | ILE | M | 1371 | 73.335 | 92.535 | 60.006 | 1.00 | 42.55 | 7 |
| ATOM | 19450 | CA | ILE | M | 1371 | 74.042 | 91.474 | 59.265 | 1.00 | 41.87 | 6 |
| ATOM | 19451 | CB | ILE | M | 1371 | 73.136 | 90.282 | 58.880 | 1.00 | 13.87 | 6 |
| ATOM | 19452 | CG2 | ILE | M | 1371 | 73.840 | 89.427 | 57.853 | 1.00 | 13.87 | 6 |
| ATOM | 19453 | CG1 | ILE | M | 1371 | 72.811 | 89.410 | 60.089 | 1.00 | 13.87 | 6 |
| ATOM | 19454 | CD1 | ILE | M | 1371 | 73.925 | 88.502 | 60.511 | 1.00 | 13.87 | 6 |
| ATOM | 19455 | C | ILE | M | 1371 | 74.532 | 92.007 | 57.940 | 1.00 | 41.53 | 6 |
| ATOM | 19456 | O | ILE | M | 1371 | 75.683 | 91.813 | 57.555 | 1.00 | 42.75 | 8 |
| ATOM | 19457 | N | VAL | M | 1372 | 73.621 | 92.661 | 57.238 | 1.00 | 19.01 | 7 |
| ATOM | 19458 | CA | VAL | M | 1372 | 73.908 | 93.217 | 55.938 | 1.00 | 19.49 | 6 |
| ATOM | 19459 | CB | VAL | M | 1372 | 72.619 | 93.685 | 55.264 | 1.00 | 29.25 | 6 |
| ATOM | 19460 | CG1 | VAL | M | 1372 | 72.874 | 94.034 | 53.819 | 1.00 | 28.47 | 6 |
| ATOM | 19461 | CG2 | VAL | M | 1372 | 71.584 | 92.612 | 55.370 | 1.00 | 29.34 | 6 |
| ATOM | 19462 | C | VAL | M | 1372 | 74.878 | 94.383 | 55.986 | 1.00 | 20.74 | 6 |
| ATOM | 19463 | O | VAL | M | 1372 | 75.778 | 94.471 | 55.142 | 1.00 | 20.78 | 8 |
| ATOM | 19464 | N | VAL | M | 1373 | 74.697 | 95.276 | 56.963 | 1.00 | 38.65 | 7 |
| ATOM | 19465 | CA | VAL | M | 1373 | 75.547 | 96.465 | 57.100 | 1.00 | 40.85 | 6 |
| ATOM | 19466 | CB | VAL | M | 1373 | 75.062 | 97.377 | 58.264 | 1.00 | 30.59 | 6 |
| ATOM | 19467 | CG1 | VAL | M | 1373 | 76.175 | 97.603 | 59.275 | 1.00 | 29.63 | 6 |
| ATOM | 19468 | CG2 | VAL | M | 1373 | 74.583 | 98.711 | 57.707 | 1.00 | 29.59 | 6 |
| ATOM | 19469 | C | VAL | M | 1373 | 77.003 | 96.086 | 57.300 | 1.00 | 43.04 | 6 |
| ATOM | 19470 | O | VAL | M | 1373 | 77.899 | 96.667 | 56.690 | 1.00 | 43.66 | 8 |
| ATOM | 19471 | N | ARG | M | 1374 | 77.234 | 95.109 | 58.160 | 1.00 | 29.36 | 7 |
| ATOM | 19472 | CA | ARG | M | 1374 | 78.578 | 94.638 | 58.406 | 1.00 | 32.53 | 6 |
| ATOM | 19473 | CB | ARG | M | 1374 | 78.532 | 93.364 | 59.232 | 1.00 | 41.74 | 6 |
| ATOM | 19474 | CG | ARG | M | 1374 | 79.839 | 92.628 | 59.294 | 1.00 | 42.39 | 6 |
| ATOM | 19475 | CD | ARG | M | 1374 | 79.582 | 91.149 | 59.467 | 1.00 | 41.99 | 6 |
| ATOM | 19476 | NE | ARG | M | 1374 | 80.068 | 90.645 | 60.747 | 1.00 | 40.86 | 7 |
| ATOM | 19477 | CZ | ARG | M | 1374 | 79.927 | 89.384 | 61.142 | 1.00 | 41.20 | 6 |
| ATOM | 19478 | NH1 | ARG | M | 1374 | 79.313 | 88.497 | 60.359 | 1.00 | 38.94 | 7 |
| ATOM | 19479 | NH2 | ARG | M | 1374 | 80.402 | 89.006 | 62.318 | 1.00 | 42.04 | 7 |
| ATOM | 19480 | C | ARG | M | 1374 | 79.251 | 94.346 | 57.069 | 1.00 | 33.82 | 6 |
| ATOM | 19481 | O | ARG | M | 1374 | 80.393 | 94.722 | 56.854 | 1.00 | 35.00 | 8 |
| ATOM | 19482 | N | GLN | M | 1375 | 78.535 | 93.677 | 56.171 | 1.00 | 26.64 | 7 |
| ATOM | 19483 | CA | GLN | M | 1375 | 79.068 | 93.325 | 54.859 | 1.00 | 27.87 | 6 |
| ATOM | 19484 | CB | GLN | M | 1375 | 78.008 | 92.590 | 54.055 | 1.00 | 36.76 | 6 |
| ATOM | 19485 | CG | GLN | M | 1375 | 77.738 | 91.208 | 54.584 | 1.00 | 37.15 | 6 |
| ATOM | 19486 | CD | GLN | M | 1375 | 78.817 | 90.238 | 54.190 | 1.00 | 37.52 | 6 |
| ATOM | 19487 | OE1 | GLN | M | 1375 | 78.969 | 89.921 | 53.012 | 1.00 | 37.07 | 8 |
| ATOM | 19488 | NE2 | GLN | M | 1375 | 79.583 | 89.762 | 55.169 | 1.00 | 36.68 | 7 |
| ATOM | 19489 | C | GLN | M | 1375 | 79.577 | 94.517 | 54.074 | 1.00 | 29.06 | 6 |
| ATOM | 19490 | O | GLN | M | 1375 | 80.518 | 94.383 | 53.289 | 1.00 | 28.43 | 8 |
| ATOM | 19491 | N | MET | M | 1376 | 78.950 | 95.673 | 54.274 | 1.00 | 42.74 | 7 |
| ATOM | 19492 | CA | MET | M | 1376 | 79.372 | 96.893 | 53.597 | 1.00 | 46.22 | 6 |
| ATOM | 19493 | CB | MET | M | 1376 | 78.395 | 98.030 | 53.865 | 1.00 | 31.58 | 6 |
| ATOM | 19494 | CG | MET | M | 1376 | 77.084 | 97.873 | 53.164 | 1.00 | 31.71 | 6 |
| ATOM | 19495 | SD | MET | M | 1376 | 76.110 | 99.368 | 53.262 | 1.00 | 31.80 | 16 |
| ATOM | 19496 | CE | MET | M | 1376 | 75.120 | 99.022 | 54.736 | 1.00 | 29.71 | 6 |
| ATOM | 19497 | C | MET | M | 1376 | 80.743 | 97.287 | 54.126 | 1.00 | 50.18 | 6 |
| ATOM | 19498 | O | MET | M | 1376 | 81.651 | 97.633 | 53.365 | 1.00 | 51.17 | 8 |
| ATOM | 19499 | N | LEU | M | 1377 | 80.884 | 97.239 | 55.445 | 1.00 | 99.63 | 7 |
| ATOM | 19500 | CA | LEU | M | 1377 | 82.144 | 97.576 | 56.085 | 1.00 | 102.98 | 6 |
| ATOM | 19501 | CB | LEU | M | 1377 | 81.899 | 98.276 | 57.431 | 1.00 | 39.74 | 6 |
| ATOM | 19502 | CG | LEU | M | 1377 | 81.791 | 99.809 | 57.455 | 1.00 | 39.67 | 6 |
| ATOM | 19503 | CD1 | LEU | M | 1377 | 82.421 | 100.388 | 56.199 | 1.00 | 37.44 | 6 |
| ATOM | 19504 | CD2 | LEU | M | 1377 | 80.353 | 100.241 | 57.554 | 1.00 | 40.91 | 6 |
| ATOM | 19505 | C | LEU | M | 1377 | 82.995 | 96.333 | 56.298 | 1.00 | 105.69 | 6 |
| ATOM | 19506 | O | LEU | M | 1377 | 83.405 | 96.039 | 57.420 | 1.00 | 106.74 | 8 |
| ATOM | 19507 | N | LYS | M | 1378 | 83.250 | 95.593 | 55.225 | 1.00 | 65.31 | 7 |
| ATOM | 19508 | CA | LYS | M | 1378 | 84.069 | 94.404 | 55.354 | 1.00 | 67.42 | 6 |
| ATOM | 19509 | CB | LYS | M | 1378 | 83.295 | 93.142 | 54.942 | 1.00 | 75.64 | 6 |
| ATOM | 19510 | CG | LYS | M | 1378 | 83.409 | 92.763 | 53.477 | 1.00 | 76.91 | 6 |
| ATOM | 19511 | CD | LYS | M | 1378 | 82.999 | 91.306 | 53.220 | 1.00 | 78.29 | 6 |
| ATOM | 19512 | CE | LYS | M | 1378 | 83.163 | 90.924 | 51.737 | 1.00 | 79.71 | 6 |
| ATOM | 19513 | NZ | LYS | M | 1378 | 82.821 | 89.504 | 51.415 | 1.00 | 81.23 | 7 |
| ATOM | 19514 | C | LYS | M | 1378 | 85.333 | 94.555 | 54.516 | 1.00 | 69.00 | 6 |
| ATOM | 19515 | O | LYS | M | 1378 | 86.295 | 93.807 | 54.707 | 1.00 | 69.64 | 8 |
| ATOM | 19516 | N | TYR | M | 1379 | 85.347 | 95.529 | 53.606 | 1.00 | 66.96 | 7 |
| ATOM | 19517 | CA | TYR | M | 1379 | 86.529 | 95.744 | 52.769 | 1.00 | 69.04 | 6 |
| ATOM | 19518 | CB | TYR | M | 1379 | 86.125 | 96.110 | 51.347 | 1.00 | 85.05 | 6 |
| ATOM | 19519 | CG | TYR | M | 1379 | 85.092 | 95.192 | 50.770 | 1.00 | 85.78 | 6 |
| ATOM | 19520 | CD1 | TYR | M | 1379 | 83.781 | 95.228 | 51.230 | 1.00 | 86.68 | 6 |
| ATOM | 19521 | CE1 | TYR | M | 1379 | 82.811 | 94.396 | 50.700 | 1.00 | 87.58 | 6 |
| ATOM | 19522 | CD2 | TYR | M | 1379 | 85.415 | 94.291 | 49.762 | 1.00 | 86.03 | 6 |
| ATOM | 19523 | CE2 | TYR | M | 1379 | 84.452 | 93.450 | 49.225 | 1.00 | 86.77 | 6 |
| ATOM | 19524 | CZ | TYR | M | 1379 | 83.150 | 93.509 | 49.700 | 1.00 | 87.40 | 6 |
| ATOM | 19525 | OH | TYR | M | 1379 | 82.173 | 92.681 | 49.197 | 1.00 | 87.15 | 8 |
| ATOM | 19526 | C | TYR | M | 1379 | 87.443 | 96.833 | 53.326 | 1.00 | 69.30 | 6 |
| ATOM | 19527 | O | TYR | M | 1379 | 86.968 | 97.813 | 53.901 | 1.00 | 68.85 | 8 |
| ATOM | 19528 | N | VAL | M | 1380 | 88.751 | 96.653 | 53.152 | 1.00 | 51.19 | 7 |
| ATOM | 19529 | CA | VAL | M | 1380 | 89.732 | 97.614 | 53.635 | 1.00 | 51.71 | 6 |
| ATOM | 19530 | CB | VAL | M | 1380 | 90.569 | 97.026 | 54.767 | 1.00 | 52.39 | 6 |
| ATOM | 19531 | CG1 | VAL | M | 1380 | 89.682 | 96.606 | 55.907 | 1.00 | 52.94 | 6 |
| ATOM | 19532 | CG2 | VAL | M | 1380 | 91.339 | 95.849 | 54.259 | 1.00 | 52.18 | 6 |
| ATOM | 19533 | C | VAL | M | 1380 | 90.676 | 98.008 | 52.509 | 1.00 | 52.86 | 6 |
| ATOM | 19534 | O | VAL | M | 1380 | 90.686 | 97.374 | 51.458 | 1.00 | 52.43 | 8 |
| ATOM | 19535 | N | GLU | M | 1381 | 91.464 | 99.059 | 52.745 | 1.00 | 89.61 | 7 |
| ATOM | 19536 | CA | GLU | M | 1381 | 92.453 | 99.579 | 51.789 | 1.00 | 90.81 | 6 |
| ATOM | 19537 | CB | GLU | M | 1381 | 92.021 | 100.954 | 51.271 | 1.00 | 82.68 | 6 |
| ATOM | 19538 | CG | GLU | M | 1381 | 92.907 | 101.529 | 50.181 | 1.00 | 83.08 | 6 |
| ATOM | 19539 | CD | GLU | M | 1381 | 92.647 | 103.012 | 49.939 | 1.00 | 84.51 | 6 |
| ATOM | 19540 | OE1 | GLU | M | 1381 | 92.955 | 103.826 | 50.835 | 1.00 | 84.88 | 8 |
| ATOM | 19541 | OE2 | GLU | M | 1381 | 92.133 | 103.367 | 48.856 | 1.00 | 85.21 | 8 |
| ATOM | 19542 | C | GLU | M | 1381 | 93.792 | 99.695 | 52.529 | 1.00 | 91.47 | 6 |
| ATOM | 19543 | O | GLU | M | 1381 | 93.973 | 100.554 | 53.394 | 1.00 | 91.06 | 8 |
| ATOM | 19544 | N | VAL | M | 1382 | 94.719 | 98.813 | 52.177 | 1.00 | 97.50 | 7 |
| ATOM | 19545 | CA | VAL | M | 1382 | 96.036 | 98.743 | 52.800 | 1.00 | 98.76 | 6 |
| ATOM | 19546 | CB | VAL | M | 1382 | 96.937 | 97.759 | 52.036 | 1.00 | 69.35 | 6 |
| ATOM | 19547 | CG1 | VAL | M | 1382 | 98.075 | 97.313 | 52.920 | 1.00 | 69.00 | 6 |
| ATOM | 19548 | CG2 | VAL | M | 1382 | 96.122 | 96.563 | 51.569 | 1.00 | 69.06 | 6 |
| ATOM | 19549 | C | VAL | M | 1382 | 96.780 | 100.071 | 52.923 | 1.00 | 100.20 | 6 |
| ATOM | 19550 | O | VAL | M | 1382 | 96.307 | 101.107 | 52.460 | 1.00 | 100.77 | 8 |
| ATOM | 19551 | N | THR | M | 1383 | 97.942 | 100.011 | 53.572 | 1.00 | 90.21 | 7 |
| ATOM | 19552 | CA | THR | M | 1383 | 98.837 | 101.150 | 53.794 | 1.00 | 91.49 | 6 |
| ATOM | 19553 | CB | THR | M | 1383 | 98.236 | 102.232 | 54.734 | 1.00 | 61.68 | 6 |
| ATOM | 19554 | OG1 | THR | M | 1383 | 97.421 | 101.611 | 55.735 | 1.00 | 60.19 | 8 |
| ATOM | 19555 | CG2 | THR | M | 1383 | 97.443 | 103.257 | 53.945 | 1.00 | 59.51 | 6 |
| ATOM | 19556 | C | THR | M | 1383 | 100.110 | 100.636 | 54.462 | 1.00 | 93.44 | 6 |
| ATOM | 19557 | O | THR | M | 1383 | 100.045 | 99.755 | 55.327 | 1.00 | 93.71 | 8 |
| ATOM | 19558 | N | ASP | M | 1384 | 101.256 | 101.191 | 54.064 | 1.00 | 125.96 | 7 |
| ATOM | 19559 | CA | ASP | M | 1384 | 102.555 | 100.802 | 54.618 | 1.00 | 127.23 | 6 |
| ATOM | 19560 | CB | ASP | M | 1384 | 102.774 | 101.496 | 55.969 | 1.00 | 208.87 | 6 |
| ATOM | 19561 | CG | ASP | M | 1384 | 104.204 | 101.378 | 56.466 | 1.00 | 208.87 | 6 |
| ATOM | 19562 | OD1 | ASP | M | 1384 | 104.690 | 100.240 | 56.631 | 1.00 | 208.87 | 8 |
| ATOM | 19563 | OD2 | ASP | M | 1384 | 104.841 | 102.429 | 56.698 | 1.00 | 208.87 | 8 |
| ATOM | 19564 | C | ASP | M | 1384 | 102.593 | 99.284 | 54.791 | 1.00 | 126.28 | 6 |
| ATOM | 19565 | O | ASP | M | 1384 | 102.663 | 98.771 | 55.909 | 1.00 | 126.47 | 8 |
| ATOM | 19566 | N | PRO | M | 1385 | 102.535 | 98.545 | 53.675 | 1.00 | 51.55 | 7 |
| ATOM | 19567 | CD | PRO | M | 1385 | 102.333 | 99.047 | 52.302 | 1.00 | 58.03 | 6 |
| ATOM | 19568 | CA | PRO | M | 1385 | 102.557 | 97.083 | 53.698 | 1.00 | 50.98 | 6 |
| ATOM | 19569 | CB | PRO | M | 1385 | 102.079 | 96.713 | 52.295 | 1.00 | 57.87 | 6 |
| ATOM | 19570 | CG | PRO | M | 1385 | 102.615 | 97.829 | 51.461 | 1.00 | 57.47 | 6 |
| ATOM | 19571 | C | PRO | M | 1385 | 103.893 | 96.426 | 54.047 | 1.00 | 51.45 | 6 |
| ATOM | 19572 | O | PRO | M | 1385 | 104.950 | 97.069 | 54.046 | 1.00 | 50.55 | 8 |
| ATOM | 19573 | N | GLY | M | 1386 | 103.814 | 95.129 | 54.339 | 1.00 | 117.85 | 7 |
| ATOM | 19574 | CA | GLY | M | 1386 | 104.977 | 94.330 | 54.685 | 1.00 | 119.09 | 6 |
| ATOM | 19575 | C | GLY | M | 1386 | 104.680 | 92.901 | 54.271 | 1.00 | 120.09 | 6 |
| ATOM | 19576 | O | GLY | M | 1386 | 103.549 | 92.435 | 54.433 | 1.00 | 120.81 | 8 |
| ATOM | 19577 | N | ASP | M | 1387 | 105.678 | 92.205 | 53.729 | 1.00 | 120.62 | 7 |
| ATOM | 19578 | CA | ASP | M | 1387 | 105.503 | 90.821 | 53.271 | 1.00 | 121.76 | 6 |
| ATOM | 19579 | CB | ASP | M | 1387 | 105.221 | 89.882 | 54.456 | 1.00 | 208.87 | 6 |
| ATOM | 19580 | CG | ASP | M | 1387 | 106.472 | 89.555 | 55.267 | 1.00 | 208.87 | 6 |
| ATOM | 19581 | OD1 | ASP | M | 1387 | 107.460 | 89.069 | 54.674 | 1.00 | 208.87 | 8 |
| ATOM | 19582 | OD2 | ASP | M | 1387 | 106.462 | 89.769 | 56.500 | 1.00 | 208.87 | 8 |
| ATOM | 19583 | C | ASP | M | 1387 | 104.346 | 90.733 | 52.275 | 1.00 | 121.46 | 6 |
| ATOM | 19584 | O | ASP | M | 1387 | 104.026 | 89.661 | 51.762 | 1.00 | 120.77 | 8 |
| ATOM | 19585 | N | SER | M | 1388 | 103.732 | 91.877 | 51.998 | 1.00 | 131.93 | 7 |
| ATOM | 19586 | CA | SER | M | 1388 | 102.603 | 91.937 | 51.089 | 1.00 | 131.21 | 6 |
| ATOM | 19587 | CB | SER | M | 1388 | 101.311 | 91.859 | 51.901 | 1.00 | 139.67 | 6 |
| ATOM | 19588 | OG | SER | M | 1388 | 101.365 | 90.785 | 52.827 | 1.00 | 139.06 | 8 |
| ATOM | 19589 | C | SER | M | 1388 | 102.651 | 93.233 | 50.278 | 1.00 | 130.81 | 6 |
| ATOM | 19590 | O | SER | M | 1388 | 102.876 | 94.311 | 50.833 | 1.00 | 130.71 | 8 |
| ATOM | 19591 | N | PRO | M | 1389 | 102.429 | 93.140 | 48.950 | 1.00 | 115.38 | 7 |
| ATOM | 19592 | CD | PRO | M | 1389 | 102.152 | 91.882 | 48.224 | 1.00 | 64.27 | 6 |
| ATOM | 19593 | CA | PRO | M | 1389 | 102.444 | 94.283 | 48.023 | 1.00 | 114.75 | 6 |
| ATOM | 19594 | CB | PRO | M | 1389 | 102.496 | 93.605 | 46.658 | 1.00 | 64.24 | 6 |
| ATOM | 19595 | CG | PRO | M | 1389 | 101.648 | 92.382 | 46.879 | 1.00 | 63.80 | 6 |
| ATOM | 19596 | C | PRO | M | 1389 | 101.261 | 95.250 | 48.129 | 1.00 | 114.34 | 6 |
| ATOM | 19597 | O | PRO | M | 1389 | 100.447 | 95.163 | 49.050 | 1.00 | 113.72 | 8 |
| ATOM | 19598 | N | LEU | M | 1390 | 101.195 | 96.179 | 47.174 | 1.00 | 77.52 | 7 |
| ATOM | 19599 | CA | LEU | M | 1390 | 100.124 | 97.172 | 47.089 | 1.00 | 77.72 | 6 |
| ATOM | 19600 | CB | LEU | M | 1390 | 98.762 | 96.471 | 47.208 | 1.00 | 165.74 | 6 |
| ATOM | 19601 | CG | LEU | M | 1390 | 98.567 | 95.145 | 46.458 | 1.00 | 167.33 | 6 |
| ATOM | 19602 | CD1 | LEU | M | 1390 | 97.185 | 94.581 | 46.761 | 1.00 | 166.78 | 6 |
| ATOM | 19603 | CD2 | LEU | M | 1390 | 98.744 | 95.358 | 44.965 | 1.00 | 168.14 | 6 |
| ATOM | 19604 | C | LEU | M | 1390 | 100.193 | 98.306 | 48.120 | 1.00 | 77.64 | 6 |
| ATOM | 19605 | O | LEU | M | 1390 | 101.166 | 98.447 | 48.870 | 1.00 | 77.42 | 8 |
| ATOM | 19606 | N | LEU | M | 1391 | 99.143 | 99.124 | 48.129 | 1.00 | 208.87 | 7 |
| ATOM | 19607 | CA | LEU | M | 1391 | 98.999 | 100.243 | 49.057 | 1.00 | 208.87 | 6 |
| ATOM | 19608 | CB | LEU | M | 1391 | 99.681 | 101.507 | 48.527 | 1.00 | 134.86 | 6 |
| ATOM | 19609 | CG | LEU | M | 1391 | 99.638 | 102.697 | 49.499 | 1.00 | 134.55 | 6 |
| ATOM | 19610 | CD1 | LEU | M | 1391 | 100.453 | 102.370 | 50.744 | 1.00 | 134.50 | 6 |
| ATOM | 19611 | CD2 | LEU | M | 1391 | 100.181 | 103.944 | 48.826 | 1.00 | 134.41 | 6 |
| ATOM | 19612 | C | LEU | M | 1391 | 97.501 | 100.489 | 49.197 | 1.00 | 208.87 | 6 |
| ATOM | 19613 | O | LEU | M | 1391 | 97.020 | 100.916 | 50.242 | 1.00 | 208.87 | 8 |
| ATOM | 19614 | N | GLU | M | 1392 | 96.776 | 100.219 | 48.116 | 1.00 | 103.79 | 7 |
| ATOM | 19615 | CA | GLU | M | 1392 | 95.324 | 100.358 | 48.068 | 1.00 | 102.15 | 6 |
| ATOM | 19616 | CB | GLU | M | 1392 | 94.922 | 101.557 | 47.195 | 1.00 | 150.32 | 6 |
| ATOM | 19617 | CG | GLU | M | 1392 | 94.969 | 102.936 | 47.888 | 1.00 | 150.57 | 6 |
| ATOM | 19618 | CD | GLU | M | 1392 | 96.376 | 103.466 | 48.148 | 1.00 | 151.09 | 6 |
| ATOM | 19619 | OE1 | GLU | M | 1392 | 97.136 | 103.659 | 47.178 | 1.00 | 151.80 | 8 |
| ATOM | 19620 | OE2 | GLU | M | 1392 | 96.721 | 103.705 | 49.325 | 1.00 | 151.25 | 8 |
| ATOM | 19621 | C | GLU | M | 1392 | 94.828 | 99.052 | 47.451 | 1.00 | 100.68 | 6 |
| ATOM | 19622 | O | GLU | M | 1392 | 94.316 | 99.034 | 46.331 | 1.00 | 100.15 | 8 |
| ATOM | 19623 | N | GLY | M | 1393 | 95.007 | 97.964 | 48.198 | 1.00 | 71.24 | 7 |
| ATOM | 19624 | CA | GLY | M | 1393 | 94.624 | 96.642 | 47.732 | 1.00 | 69.11 | 6 |
| ATOM | 19625 | C | GLY | M | 1393 | 93.153 | 96.277 | 47.810 | 1.00 | 68.17 | 6 |
| ATOM | 19626 | O | GLY | M | 1393 | 92.735 | 95.292 | 47.196 | 1.00 | 67.32 | 8 |
| ATOM | 19627 | N | GLN | M | 1394 | 92.363 | 97.053 | 48.547 | 1.00 | 37.61 | 7 |
| ATOM | 19628 | CA | GLN | M | 1394 | 90.941 | 96.766 | 48.678 | 1.00 | 37.13 | 6 |
| ATOM | 19629 | CB | GLN | M | 1394 | 90.204 | 97.214 | 47.407 | 1.00 | 130.66 | 6 |
| ATOM | 19630 | CG | GLN | M | 1394 | 88.687 | 97.294 | 47.547 | 1.00 | 132.41 | 6 |
| ATOM | 19631 | CD | GLN | M | 1394 | 88.049 | 98.236 | 46.532 | 1.00 | 132.57 | 6 |
| ATOM | 19632 | OE1 | GLN | M | 1394 | 88.172 | 98.046 | 45.323 | 1.00 | 132.50 | 8 |
| ATOM | 19633 | NE2 | GLN | M | 1394 | 87.363 | 99.260 | 47.028 | 1.00 | 132.53 | 7 |
| ATOM | 19634 | C | GLN | M | 1394 | 90.731 | 95.260 | 48.945 | 1.00 | 36.70 | 6 |
| ATOM | 19635 | O | GLN | M | 1394 | 90.282 | 94.500 | 48.071 | 1.00 | 35.41 | 8 |
| ATOM | 19636 | N | VAL | M | 1395 | 91.058 | 94.858 | 50.177 | 1.00 | 42.77 | 7 |
| ATOM | 19637 | CA | VAL | M | 1395 | 90.960 | 93.473 | 50.641 | 1.00 | 43.23 | 6 |
| ATOM | 19638 | CB | VAL | M | 1395 | 92.363 | 92.949 | 51.094 | 1.00 | 71.52 | 6 |
| ATOM | 19639 | CG1 | VAL | M | 1395 | 92.275 | 91.518 | 51.576 | 1.00 | 71.52 | 6 |
| ATOM | 19640 | CG2 | VAL | M | 1395 | 93.344 | 93.036 | 49.942 | 1.00 | 71.88 | 6 |
| ATOM | 19641 | C | VAL | M | 1395 | 89.977 | 93.339 | 51.806 | 1.00 | 43.82 | 6 |
| ATOM | 19642 | O | VAL | M | 1395 | 89.641 | 94.318 | 52.466 | 1.00 | 43.49 | 8 |
| ATOM | 19643 | N | ALA | M | 1396 | 89.537 | 92.109 | 52.049 | 1.00 | 99.50 | 7 |
| ATOM | 19644 | CA | ALA | M | 1396 | 88.586 | 91.800 | 53.111 | 1.00 | 101.35 | 6 |
| ATOM | 19645 | CB | ALA | M | 1396 | 88.011 | 90.402 | 52.887 | 1.00 | 66.86 | 6 |
| ATOM | 19646 | C | ALA | M | 1396 | 89.165 | 91.912 | 54.523 | 1.00 | 102.80 | 6 |
| ATOM | 19647 | O | ALA | M | 1396 | 89.981 | 91.092 | 54.945 | 1.00 | 103.86 | 8 |
| ATOM | 19648 | N | GLU | M | 1397 | 88.709 | 92.929 | 55.248 | 1.00 | 70.78 | 7 |
| ATOM | 19649 | CA | GLU | M | 1397 | 89.148 | 93.201 | 56.614 | 1.00 | 71.98 | 6 |
| ATOM | 19650 | CB | GLU | M | 1397 | 88.065 | 93.969 | 57.363 | 1.00 | 95.20 | 6 |
| ATOM | 19651 | CG | GLU | M | 1397 | 88.376 | 94.163 | 58.830 | 1.00 | 94.18 | 6 |
| ATOM | 19652 | CD | GLU | M | 1397 | 87.126 | 94.359 | 59.656 | 1.00 | 93.94 | 6 |
| ATOM | 19653 | OE1 | GLU | M | 1397 | 86.408 | 95.355 | 59.418 | 1.00 | 94.32 | 8 |
| ATOM | 19654 | OE2 | GLU | M | 1397 | 86.855 | 93.514 | 60.539 | 1.00 | 93.97 | 8 |
| ATOM | 19655 | C | GLU | M | 1397 | 89.557 | 91.993 | 57.456 | 1.00 | 73.50 | 6 |
| ATOM | 19656 | O | GLU | M | 1397 | 90.508 | 92.079 | 58.226 | 1.00 | 73.72 | 8 |
| ATOM | 19657 | N | LYS | M | 1398 | 88.839 | 90.881 | 57.341 | 1.00 | 143.02 | 7 |
| ATOM | 19658 | CA | LYS | M | 1398 | 89.195 | 89.705 | 58.129 | 1.00 | 145.05 | 6 |
| ATOM | 19659 | CB | LYS | M | 1398 | 88.014 | 88.737 | 58.253 | 1.00 | 121.91 | 6 |
| ATOM | 19660 | CG | LYS | M | 1398 | 88.354 | 87.439 | 58.996 | 1.00 | 122.19 | 6 |
| ATOM | 19661 | CD | LYS | M | 1398 | 89.018 | 87.707 | 60.352 | 1.00 | 122.44 | 6 |
| ATOM | 19662 | CE | LYS | M | 1398 | 89.523 | 86.420 | 61.007 | 1.00 | 123.06 | 6 |
| ATOM | 19663 | NZ | LYS | M | 1398 | 90.331 | 86.670 | 62.240 | 1.00 | 123.08 | 7 |
| ATOM | 19664 | C | LYS | M | 1398 | 90.379 | 88.992 | 57.507 | 1.00 | 146.10 | 6 |
| ATOM | 19665 | O | LYS | M | 1398 | 91.278 | 88.532 | 58.210 | 1.00 | 146.57 | 8 |
| ATOM | 19666 | N | TRP | M | 1399 | 90.378 | 88.893 | 56.184 | 1.00 | 76.62 | 7 |
| ATOM | 19667 | CA | TRP | M | 1399 | 91.485 | 88.251 | 55.496 | 1.00 | 77.83 | 6 |
| ATOM | 19668 | CB | TRP | M | 1399 | 91.315 | 88.395 | 53.980 | 1.00 | 198.31 | 6 |
| ATOM | 19669 | CG | TRP | M | 1399 | 92.033 | 87.349 | 53.176 | 1.00 | 199.94 | 6 |
| ATOM | 19670 | CD2 | TRP | M | 1399 | 93.166 | 87.545 | 52.317 | 1.00 | 200.76 | 6 |
| ATOM | 19671 | CE2 | TRP | M | 1399 | 93.497 | 86.285 | 51.768 | 1.00 | 201.10 | 6 |
| ATOM | 19672 | CE3 | TRP | M | 1399 | 93.935 | 88.661 | 51.960 | 1.00 | 200.98 | 6 |
| ATOM | 19673 | CD1 | TRP | M | 1399 | 91.733 | 86.018 | 53.113 | 1.00 | 200.52 | 6 |
| ATOM | 19674 | NE1 | TRP | M | 1399 | 92.607 | 85.372 | 52.270 | 1.00 | 201.06 | 7 |
| ATOM | 19675 | CZ2 | TRP | M | 1399 | 94.564 | 86.111 | 50.877 | 1.00 | 201.30 | 6 |
| ATOM | 19676 | CZ3 | TRP | M | 1399 | 94.997 | 88.487 | 51.072 | 1.00 | 200.99 | 6 |
| ATOM | 19677 | CH2 | TRP | M | 1399 | 95.300 | 87.221 | 50.543 | 1.00 | 201.01 | 6 |
| ATOM | 19678 | C | TRP | M | 1399 | 92.725 | 89.018 | 55.964 | 1.00 | 78.09 | 6 |
| ATOM | 19679 | O | TRP | M | 1399 | 93.818 | 88.464 | 56.101 | 1.00 | 77.43 | 8 |
| ATOM | 19680 | N | ASP | M | 1400 | 92.517 | 90.304 | 56.234 | 1.00 | 112.62 | 7 |
| ATOM | 19681 | CA | ASP | M | 1400 | 93.570 | 91.207 | 56.680 | 1.00 | 113.56 | 6 |
| ATOM | 19682 | CB | ASP | M | 1400 | 93.139 | 92.648 | 56.440 | 1.00 | 96.52 | 6 |
| ATOM | 19683 | CG | ASP | M | 1400 | 94.258 | 93.505 | 55.926 | 1.00 | 96.52 | 6 |
| ATOM | 19684 | OD1 | ASP | M | 1400 | 94.606 | 93.383 | 54.732 | 1.00 | 95.82 | 8 |
| ATOM | 19685 | OD2 | ASP | M | 1400 | 94.798 | 94.302 | 56.713 | 1.00 | 97.49 | 8 |
| ATOM | 19686 | C | ASP | M | 1400 | 93.886 | 91.026 | 58.159 | 1.00 | 114.67 | 6 |
| ATOM | 19687 | O | ASP | M | 1400 | 94.914 | 91.496 | 58.640 | 1.00 | 114.97 | 8 |
| ATOM | 19688 | N | VAL | M | 1401 | 92.990 | 90.362 | 58.880 | 1.00 | 156.39 | 7 |
| ATOM | 19689 | CA | VAL | M | 1401 | 93.189 | 90.126 | 60.305 | 1.00 | 157.96 | 6 |
| ATOM | 19690 | CB | VAL | M | 1401 | 91.852 | 89.849 | 61.022 | 1.00 | 147.16 | 6 |
| ATOM | 19691 | CG1 | VAL | M | 1401 | 92.102 | 89.498 | 62.489 | 1.00 | 148.29 | 6 |
| ATOM | 19692 | CG2 | VAL | M | 1401 | 90.950 | 91.071 | 60.916 | 1.00 | 146.45 | 6 |
| ATOM | 19693 | C | VAL | M | 1401 | 94.118 | 88.937 | 60.503 | 1.00 | 159.07 | 6 |
| ATOM | 19694 | O | VAL | M | 1401 | 94.638 | 88.715 | 61.597 | 1.00 | 159.94 | 8 |
| ATOM | 19695 | N | GLU | M | 1402 | 94.320 | 88.173 | 59.435 | 1.00 | 119.47 | 7 |
| ATOM | 19696 | CA | GLU | M | 1402 | 95.207 | 87.017 | 59.482 | 1.00 | 119.80 | 6 |
| ATOM | 19697 | CB | GLU | M | 1402 | 94.934 | 86.094 | 58.291 | 1.00 | 206.10 | 6 |
| ATOM | 19698 | CG | GLU | M | 1402 | 93.580 | 85.408 | 58.329 | 1.00 | 206.18 | 6 |
| ATOM | 19699 | CD | GLU | M | 1402 | 93.367 | 84.626 | 59.609 | 1.00 | 207.21 | 6 |
| ATOM | 19700 | OE1 | GLU | M | 1402 | 93.128 | 85.258 | 60.660 | 1.00 | 206.93 | 8 |
| ATOM | 19701 | OE2 | GLU | M | 1402 | 93.450 | 83.380 | 59.566 | 1.00 | 208.48 | 8 |
| ATOM | 19702 | C | GLU | M | 1402 | 96.663 | 87.488 | 59.452 | 1.00 | 119.67 | 6 |
| ATOM | 19703 | O | GLU | M | 1402 | 97.503 | 87.010 | 60.219 | 1.00 | 119.15 | 8 |
| ATOM | 19704 | N | ALA | M | 1403 | 96.948 | 88.433 | 58.558 | 1.00 | 136.01 | 7 |
| ATOM | 19705 | CA | ALA | M | 1403 | 98.287 | 88.992 | 58.411 | 1.00 | 136.28 | 6 |
| ATOM | 19706 | CB | ALA | M | 1403 | 98.255 | 90.165 | 57.447 | 1.00 | 77.89 | 6 |
| ATOM | 19707 | C | ALA | M | 1403 | 98.812 | 89.446 | 59.764 | 1.00 | 136.20 | 6 |
| ATOM | 19708 | O | ALA | M | 1403 | 99.704 | 88.819 | 60.331 | 1.00 | 136.35 | 8 |
| ATOM | 19709 | N | LEU | M | 1404 | 98.249 | 90.536 | 60.278 | 1.00 | 93.65 | 7 |
| ATOM | 19710 | CA | LEU | M | 1404 | 98.653 | 91.078 | 61.573 | 1.00 | 94.44 | 6 |
| ATOM | 19711 | CB | LEU | M | 1404 | 97.762 | 92.262 | 61.954 | 1.00 | 189.17 | 6 |
| ATOM | 19712 | CG | LEU | M | 1404 | 98.138 | 93.611 | 61.344 | 1.00 | 189.28 | 6 |
| ATOM | 19713 | CD1 | LEU | M | 1404 | 97.128 | 94.665 | 61.764 | 1.00 | 189.56 | 6 |
| ATOM | 19714 | CD2 | LEU | M | 1404 | 99.534 | 94.000 | 61.808 | 1.00 | 189.25 | 6 |
| ATOM | 19715 | C | LEU | M | 1404 | 98.639 | 90.061 | 62.707 | 1.00 | 94.69 | 6 |
| ATOM | 19716 | O | LEU | M | 1404 | 98.786 | 90.425 | 63.869 | 1.00 | 94.40 | 8 |
| ATOM | 19717 | N | ASN | M | 1405 | 98.441 | 88.794 | 62.368 | 1.00 | 208.26 | 7 |
| ATOM | 19718 | CA | ASN | M | 1405 | 98.434 | 87.722 | 63.355 | 1.00 | 208.87 | 6 |
| ATOM | 19719 | CB | ASN | M | 1405 | 97.093 | 86.986 | 63.348 | 1.00 | 182.53 | 6 |
| ATOM | 19720 | CG | ASN | M | 1405 | 97.168 | 85.636 | 64.044 | 1.00 | 183.31 | 6 |
| ATOM | 19721 | OD1 | ASN | M | 1405 | 97.519 | 85.550 | 65.221 | 1.00 | 182.88 | 8 |
| ATOM | 19722 | ND2 | ASN | M | 1405 | 96.845 | 84.574 | 63.314 | 1.00 | 183.70 | 7 |
| ATOM | 19723 | C | ASN | M | 1405 | 99.543 | 86.740 | 63.016 | 1.00 | 208.87 | 6 |
| ATOM | 19724 | O | ASN | M | 1405 | 100.505 | 86.581 | 63.769 | 1.00 | 208.87 | 8 |
| ATOM | 19725 | N | GLU | M | 1406 | 99.396 | 86.085 | 61.870 | 1.00 | 92.51 | 7 |
| ATOM | 19726 | CA | GLU | M | 1406 | 100.372 | 85.112 | 61.407 | 1.00 | 92.09 | 6 |
| ATOM | 19727 | CB | GLU | M | 1406 | 99.851 | 84.446 | 60.133 | 1.00 | 123.09 | 6 |
| ATOM | 19728 | CG | GLU | M | 1406 | 100.374 | 83.048 | 59.874 | 1.00 | 122.99 | 6 |
| ATOM | 19729 | CD | GLU | M | 1406 | 99.567 | 82.333 | 58.807 | 1.00 | 123.10 | 6 |
| ATOM | 19730 | OE1 | GLU | M | 1406 | 99.438 | 82.876 | 57.688 | 1.00 | 122.69 | 8 |
| ATOM | 19731 | OE2 | GLU | M | 1406 | 99.057 | 81.229 | 59.086 | 1.00 | 123.08 | 8 |
| ATOM | 19732 | C | GLU | M | 1406 | 101.678 | 85.854 | 61.143 | 1.00 | 91.83 | 6 |
| ATOM | 19733 | O | GLU | M | 1406 | 102.747 | 85.250 | 61.082 | 1.00 | 91.75 | 8 |
| ATOM | 19734 | N | ARG | M | 1407 | 101.574 | 87.174 | 61.001 | 1.00 | 160.99 | 7 |
| ATOM | 19735 | CA | ARG | M | 1407 | 102.732 | 88.027 | 60.759 | 1.00 | 161.66 | 6 |
| ATOM | 19736 | CB | ARG | M | 1407 | 102.457 | 88.988 | 59.600 | 1.00 | 168.21 | 6 |
| ATOM | 19737 | CG | ARG | M | 1407 | 102.290 | 88.326 | 58.242 | 1.00 | 169.31 | 6 |
| ATOM | 19738 | CD | ARG | M | 1407 | 103.610 | 87.786 | 57.713 | 1.00 | 170.45 | 6 |
| ATOM | 19739 | NE | ARG | M | 1407 | 103.507 | 87.398 | 56.308 | 1.00 | 172.31 | 7 |
| ATOM | 19740 | CZ | ARG | M | 1407 | 104.522 | 86.939 | 55.584 | 1.00 | 172.51 | 6 |
| ATOM | 19741 | NH1 | ARG | M | 1407 | 105.723 | 86.808 | 56.131 | 1.00 | 172.38 | 7 |
| ATOM | 19742 | NH2 | ARG | M | 1407 | 104.339 | 86.619 | 54.310 | 1.00 | 172.91 | 7 |
| ATOM | 19743 | C | ARG | M | 1407 | 103.085 | 88.835 | 62.007 | 1.00 | 161.67 | 6 |
| ATOM | 19744 | O | ARG | M | 1407 | 104.172 | 89.409 | 62.094 | 1.00 | 161.67 | 8 |
| ATOM | 19745 | N | LEU | M | 1408 | 102.163 | 88.890 | 62.966 | 1.00 | 105.75 | 7 |
| ATOM | 19746 | CA | LEU | M | 1408 | 102.406 | 89.624 | 64.204 | 1.00 | 105.26 | 6 |
| ATOM | 19747 | CB | LEU | M | 1408 | 101.145 | 89.680 | 65.063 | 1.00 | 208.87 | 6 |
| ATOM | 19748 | CG | LEU | M | 1408 | 101.263 | 90.523 | 66.338 | 1.00 | 208.87 | 6 |
| ATOM | 19749 | CD1 | LEU | M | 1408 | 101.455 | 91.989 | 65.973 | 1.00 | 208.87 | 6 |
| ATOM | 19750 | CD2 | LEU | M | 1408 | 100.013 | 90.350 | 67.183 | 1.00 | 208.87 | 6 |
| ATOM | 19751 | C | LEU | M | 1408 | 103.503 | 88.924 | 64.991 | 1.00 | 104.97 | 6 |
| ATOM | 19752 | O | LEU | M | 1408 | 104.010 | 89.459 | 65.978 | 1.00 | 104.90 | 8 |
| ATOM | 19753 | N | ILE | M | 1409 | 103.850 | 87.715 | 64.555 | 1.00 | 118.87 | 7 |
| ATOM | 19754 | CA | ILE | M | 1409 | 104.892 | 86.924 | 65.199 | 1.00 | 118.29 | 6 |
| ATOM | 19755 | CB | ILE | M | 1409 | 104.837 | 85.462 | 64.733 | 1.00 | 157.06 | 6 |
| ATOM | 19756 | CG2 | ILE | M | 1409 | 105.718 | 84.599 | 65.616 | 1.00 | 157.66 | 6 |
| ATOM | 19757 | CG1 | ILE | M | 1409 | 103.390 | 84.960 | 64.779 | 1.00 | 157.31 | 6 |
| ATOM | 19758 | CD1 | ILE | M | 1409 | 102.717 | 85.121 | 66.129 | 1.00 | 157.10 | 6 |
| ATOM | 19759 | C | ILE | M | 1409 | 106.232 | 87.529 | 64.807 | 1.00 | 118.31 | 6 |
| ATOM | 19760 | O | ILE | M | 1409 | 107.199 | 86.823 | 64.520 | 1.00 | 118.52 | 8 |
| ATOM | 19761 | N | ALA | M | 1410 | 106.252 | 88.857 | 64.788 | 1.00 | 110.75 | 7 |
| ATOM | 19762 | CA | ALA | M | 1410 | 107.421 | 89.648 | 64.438 | 1.00 | 110.99 | 6 |
| ATOM | 19763 | CB | ALA | M | 1410 | 107.612 | 89.653 | 62.925 | 1.00 | 75.19 | 6 |
| ATOM | 19764 | C | ALA | M | 1410 | 107.177 | 91.071 | 64.942 | 1.00 | 111.09 | 6 |
| ATOM | 19765 | O | ALA | M | 1410 | 107.233 | 92.003 | 64.118 | 1.00 | 111.24 | 8 |
| ATOM | 19766 | OXT | ALA | M | 1410 | 106.923 | 91.240 | 66.154 | 1.00 | 76.43 | 8 |
| ATOM | 19767 | CB | VAL | N | 1414 | 106.256 | 92.922 | 60.034 | 1.00 | 103.76 | 6 |
| ATOM | 19768 | CG1 | VAL | N | 1414 | 106.136 | 93.593 | 58.675 | 1.00 | 104.04 | 6 |
| ATOM | 19769 | CG2 | VAL | N | 1414 | 107.662 | 92.370 | 60.235 | 1.00 | 104.28 | 6 |
| ATOM | 19770 | C | VAL | N | 1414 | 104.501 | 94.461 | 60.929 | 1.00 | 121.75 | 6 |
| ATOM | 19771 | O | VAL | N | 1414 | 103.622 | 93.742 | 60.445 | 1.00 | 121.37 | 8 |
| ATOM | 19772 | N | VAL | N | 1414 | 106.020 | 93.308 | 62.501 | 1.00 | 122.57 | 7 |
| ATOM | 19773 | CA | VAL | N | 1414 | 105.916 | 93.935 | 61.153 | 1.00 | 122.38 | 6 |
| ATOM | 19774 | N | PRO | N | 1415 | 104.266 | 95.733 | 61.278 | 1.00 | 166.28 | 7 |
| ATOM | 19775 | CD | PRO | N | 1415 | 105.228 | 96.695 | 61.849 | 1.00 | 86.61 | 6 |
| ATOM | 19776 | CA | PRO | N | 1415 | 102.948 | 96.349 | 61.113 | 1.00 | 165.75 | 6 |
| ATOM | 19777 | CB | PRO | N | 1415 | 103.236 | 97.831 | 61.341 | 1.00 | 86.35 | 6 |
| ATOM | 19778 | CG | PRO | N | 1415 | 104.316 | 97.786 | 62.381 | 1.00 | 86.42 | 6 |
| ATOM | 19779 | C | PRO | N | 1415 | 102.290 | 96.081 | 59.760 | 1.00 | 165.13 | 6 |
| ATOM | 19780 | O | PRO | N | 1415 | 102.760 | 96.546 | 58.721 | 1.00 | 165.22 | 8 |
| ATOM | 19781 | N | VAL | N | 1416 | 101.204 | 95.313 | 59.792 | 1.00 | 118.55 | 7 |
| ATOM | 19782 | CA | VAL | N | 1416 | 100.429 | 94.985 | 58.598 | 1.00 | 117.25 | 6 |
| ATOM | 19783 | CB | VAL | N | 1416 | 100.067 | 93.493 | 58.552 | 1.00 | 85.54 | 6 |
| ATOM | 19784 | CG1 | VAL | N | 1416 | 99.290 | 93.187 | 57.282 | 1.00 | 85.07 | 6 |
| ATOM | 19785 | CG2 | VAL | N | 1416 | 101.333 | 92.652 | 58.647 | 1.00 | 85.05 | 6 |
| ATOM | 19786 | C | VAL | N | 1416 | 99.135 | 95.774 | 58.709 | 1.00 | 116.02 | 6 |
| ATOM | 19787 | O | VAL | N | 1416 | 98.072 | 95.194 | 58.912 | 1.00 | 115.60 | 8 |
| ATOM | 19788 | N | ALA | N | 1417 | 99.228 | 97.094 | 58.581 | 1.00 | 100.17 | 7 |
| ATOM | 19789 | CA | ALA | N | 1417 | 98.059 | 97.952 | 58.713 | 1.00 | 98.01 | 6 |
| ATOM | 19790 | CB | ALA | N | 1417 | 98.472 | 99.329 | 59.214 | 1.00 | 53.64 | 6 |
| ATOM | 19791 | C | ALA | N | 1417 | 97.245 | 98.091 | 57.443 | 1.00 | 96.92 | 6 |
| ATOM | 19792 | O | ALA | N | 1417 | 97.740 | 97.847 | 56.342 | 1.00 | 97.36 | 8 |
| ATOM | 19793 | N | TRP | N | 1418 | 95.990 | 98.498 | 57.625 | 1.00 | 76.29 | 7 |
| ATOM | 19794 | CA | TRP | N | 1418 | 95.035 | 98.690 | 56.541 | 1.00 | 73.93 | 6 |
| ATOM | 19795 | CB | TRP | N | 1418 | 94.174 | 97.438 | 56.414 | 1.00 | 79.43 | 6 |
| ATOM | 19796 | CG | TRP | N | 1418 | 93.206 | 97.291 | 57.549 | 1.00 | 80.05 | 6 |
| ATOM | 19797 | CD2 | TRP | N | 1418 | 93.369 | 96.486 | 58.715 | 1.00 | 80.30 | 6 |
| ATOM | 19798 | CE2 | TRP | N | 1418 | 92.245 | 96.713 | 59.541 | 1.00 | 80.89 | 6 |
| ATOM | 19799 | CE3 | TRP | N | 1418 | 94.354 | 95.593 | 59.147 | 1.00 | 80.13 | 6 |
| ATOM | 19800 | CD1 | TRP | N | 1418 | 92.022 | 97.955 | 57.708 | 1.00 | 80.75 | 6 |
| ATOM | 19801 | NE1 | TRP | N | 1418 | 91.439 | 97.615 | 58.900 | 1.00 | 81.20 | 7 |
| ATOM | 19802 | CZ2 | TRP | N | 1418 | 92.081 | 96.079 | 60.777 | 1.00 | 81.08 | 6 |
| ATOM | 19803 | CZ3 | TRP | N | 1418 | 94.192 | 94.960 | 60.378 | 1.00 | 80.13 | 6 |
| ATOM | 19804 | CH2 | TRP | N | 1418 | 93.063 | 95.208 | 61.177 | 1.00 | 80.82 | 6 |
| ATOM | 19805 | C | TRP | N | 1418 | 94.158 | 99.872 | 56.948 | 1.00 | 72.31 | 6 |
| ATOM | 19806 | O | TRP | N | 1418 | 94.292 | 100.371 | 58.060 | 1.00 | 71.80 | 8 |
| ATOM | 19807 | N | LYS | N | 1419 | 93.259 | 100.305 | 56.063 | 1.00 | 83.62 | 7 |
| ATOM | 19808 | CA | LYS | N | 1419 | 92.335 | 101.407 | 56.362 | 1.00 | 82.32 | 6 |
| ATOM | 19809 | CB | LYS | N | 1419 | 92.670 | 102.625 | 55.498 | 1.00 | 106.59 | 6 |
| ATOM | 19810 | CG | LYS | N | 1419 | 93.997 | 103.267 | 55.874 | 1.00 | 110.34 | 6 |
| ATOM | 19811 | CD | LYS | N | 1419 | 94.270 | 104.562 | 55.113 | 1.00 | 112.26 | 6 |
| ATOM | 19812 | CE | LYS | N | 1419 | 95.536 | 105.254 | 55.637 | 1.00 | 113.28 | 6 |
| ATOM | 19813 | NZ | LYS | N | 1419 | 95.935 | 106.457 | 54.845 | 1.00 | 113.93 | 7 |
| ATOM | 19814 | C | LYS | N | 1419 | 90.882 | 100.965 | 56.121 | 1.00 | 80.11 | 6 |
| ATOM | 19815 | O | LYS | N | 1419 | 90.633 | 100.112 | 55.279 | 1.00 | 80.55 | 8 |
| ATOM | 19816 | N | PRO | N | 1420 | 89.909 | 101.509 | 56.882 | 1.00 | 41.53 | 7 |
| ATOM | 19817 | CD | PRO | N | 1420 | 90.094 | 102.216 | 58.162 | 1.00 | 101.26 | 6 |
| ATOM | 19818 | CA | PRO | N | 1420 | 88.496 | 101.132 | 56.703 | 1.00 | 38.15 | 6 |
| ATOM | 19819 | CB | PRO | N | 1420 | 87.878 | 101.431 | 58.067 | 1.00 | 98.29 | 6 |
| ATOM | 19820 | CG | PRO | N | 1420 | 88.671 | 102.592 | 58.537 | 1.00 | 99.71 | 6 |
| ATOM | 19821 | C | PRO | N | 1420 | 87.755 | 101.838 | 55.562 | 1.00 | 35.21 | 6 |
| ATOM | 19822 | O | PRO | N | 1420 | 87.531 | 103.043 | 55.594 | 1.00 | 34.19 | 8 |
| ATOM | 19823 | N | LEU | N | 1421 | 87.369 | 101.058 | 54.560 | 1.00 | 53.01 | 7 |
| ATOM | 19824 | CA | LEU | N | 1421 | 86.670 | 101.567 | 53.389 | 1.00 | 50.78 | 6 |
| ATOM | 19825 | CB | LEU | N | 1421 | 87.303 | 100.962 | 52.131 | 1.00 | 51.46 | 6 |
| ATOM | 19826 | CG | LEU | N | 1421 | 86.696 | 101.213 | 50.752 | 1.00 | 51.59 | 6 |
| ATOM | 19827 | CD1 | LEU | N | 1421 | 86.407 | 102.688 | 50.544 | 1.00 | 52.13 | 6 |
| ATOM | 19828 | CD2 | LEU | N | 1421 | 87.664 | 100.704 | 49.705 | 1.00 | 52.24 | 6 |
| ATOM | 19829 | C | LEU | N | 1421 | 85.173 | 101.238 | 53.480 | 1.00 | 49.30 | 6 |
| ATOM | 19830 | O | LEU | N | 1421 | 84.780 | 100.282 | 54.155 | 1.00 | 49.32 | 8 |
| ATOM | 19831 | N | LEU | N | 1422 | 84.344 | 102.044 | 52.812 | 1.00 | 79.90 | 7 |
| ATOM | 19832 | CA | LEU | N | 1422 | 82.888 | 101.871 | 52.828 | 1.00 | 78.18 | 6 |
| ATOM | 19833 | CB | LEU | N | 1422 | 82.203 | 103.183 | 53.222 | 1.00 | 42.96 | 6 |
| ATOM | 19834 | CG | LEU | N | 1422 | 80.812 | 103.115 | 53.854 | 1.00 | 40.12 | 6 |
| ATOM | 19835 | CD1 | LEU | N | 1422 | 80.959 | 102.831 | 55.335 | 1.00 | 39.70 | 6 |
| ATOM | 19836 | CD2 | LEU | N | 1422 | 80.078 | 104.427 | 53.658 | 1.00 | 38.75 | 6 |
| ATOM | 19837 | C | LEU | N | 1422 | 82.383 | 101.454 | 51.456 | 1.00 | 78.42 | 6 |
| ATOM | 19838 | O | LEU | N | 1422 | 82.441 | 102.230 | 50.501 | 1.00 | 78.07 | 8 |
| ATOM | 19839 | N | MET | N | 1423 | 81.876 | 100.230 | 51.367 | 1.00 | 59.38 | 7 |
| ATOM | 19840 | CA | MET | N | 1423 | 81.366 | 99.709 | 50.108 | 1.00 | 58.29 | 6 |
| ATOM | 19841 | CB | MET | N | 1423 | 81.770 | 98.244 | 49.942 | 1.00 | 30.39 | 6 |
| ATOM | 19842 | CG | MET | N | 1423 | 83.253 | 97.998 | 49.946 | 1.00 | 27.16 | 6 |
| ATOM | 19843 | SD | MET | N | 1423 | 84.006 | 98.405 | 48.393 | 1.00 | 25.19 | 16 |
| ATOM | 19844 | CE | MET | N | 1423 | 84.526 | 96.839 | 47.818 | 1.00 | 23.94 | 6 |
| ATOM | 19845 | C | MET | N | 1423 | 79.852 | 99.803 | 50.039 | 1.00 | 59.24 | 6 |
| ATOM | 19846 | O | MET | N | 1423 | 79.163 | 99.562 | 51.031 | 1.00 | 59.56 | 8 |
| ATOM | 19847 | N | GLY | N | 1424 | 79.341 | 100.170 | 48.868 | 1.00 | 56.07 | 7 |
| ATOM | 19848 | CA | GLY | N | 1424 | 77.904 | 100.230 | 48.683 | 1.00 | 56.69 | 6 |
| ATOM | 19849 | C | GLY | N | 1424 | 77.515 | 98.773 | 48.740 | 1.00 | 57.24 | 6 |
| ATOM | 19850 | O | GLY | N | 1424 | 78.365 | 97.920 | 48.510 | 1.00 | 57.38 | 8 |
| ATOM | 19851 | N | VAL | N | 1425 | 76.268 | 98.455 | 49.047 | 1.00 | 58.10 | 7 |
| ATOM | 19852 | CA | VAL | N | 1425 | 75.913 | 97.050 | 49.123 | 1.00 | 59.55 | 6 |
| ATOM | 19853 | CB | VAL | N | 1425 | 74.447 | 96.878 | 49.563 | 1.00 | 27.26 | 6 |
| ATOM | 19854 | CG1 | VAL | N | 1425 | 73.583 | 96.471 | 48.399 | 1.00 | 28.54 | 6 |
| ATOM | 19855 | CG2 | VAL | N | 1425 | 74.368 | 95.867 | 50.678 | 1.00 | 26.37 | 6 |
| ATOM | 19856 | C | VAL | N | 1425 | 76.168 | 96.385 | 47.770 | 1.00 | 62.07 | 6 |
| ATOM | 19857 | O | VAL | N | 1425 | 76.577 | 95.228 | 47.699 | 1.00 | 63.00 | 8 |
| ATOM | 19858 | N | THR | N | 1426 | 75.954 | 97.138 | 46.695 | 1.00 | 27.25 | 7 |
| ATOM | 19859 | CA | THR | N | 1426 | 76.164 | 96.630 | 45.349 | 1.00 | 28.60 | 6 |
| ATOM | 19860 | CB | THR | N | 1426 | 75.627 | 97.621 | 44.309 | 1.00 | 48.38 | 6 |
| ATOM | 19861 | OG1 | THR | N | 1426 | 74.200 | 97.684 | 44.406 | 1.00 | 47.95 | 8 |
| ATOM | 19862 | CG2 | THR | N | 1426 | 76.014 | 97.193 | 42.907 | 1.00 | 49.21 | 6 |
| ATOM | 19863 | C | THR | N | 1426 | 77.642 | 96.367 | 45.077 | 1.00 | 31.31 | 6 |
| ATOM | 19864 | O | THR | N | 1426 | 78.036 | 95.234 | 44.817 | 1.00 | 31.61 | 8 |
| ATOM | 19865 | N | LYS | N | 1427 | 78.456 | 97.415 | 45.145 | 1.00 | 97.03 | 7 |
| ATOM | 19866 | CA | LYS | N | 1427 | 79.890 | 97.287 | 44.907 | 1.00 | 100.40 | 6 |
| ATOM | 19867 | CB | LYS | N | 1427 | 80.598 | 98.631 | 45.062 | 1.00 | 125.28 | 6 |
| ATOM | 19868 | CG | LYS | N | 1427 | 80.339 | 99.617 | 43.948 | 1.00 | 127.97 | 6 |
| ATOM | 19869 | CD | LYS | N | 1427 | 81.144 | 100.886 | 44.164 | 1.00 | 129.49 | 6 |
| ATOM | 19870 | CE | LYS | N | 1427 | 80.893 | 101.901 | 43.063 | 1.00 | 130.24 | 6 |
| ATOM | 19871 | NZ | LYS | N | 1427 | 81.740 | 103.112 | 43.239 | 1.00 | 131.93 | 7 |
| ATOM | 19872 | C | LYS | N | 1427 | 80.541 | 96.306 | 45.858 | 1.00 | 102.13 | 6 |
| ATOM | 19873 | O | LYS | N | 1427 | 81.758 | 96.170 | 45.856 | 1.00 | 102.94 | 8 |
| ATOM | 19874 | N | SER | N | 1428 | 79.740 | 95.638 | 46.681 | 1.00 | 55.35 | 7 |
| ATOM | 19875 | CA | SER | N | 1428 | 80.275 | 94.674 | 47.632 | 1.00 | 56.80 | 6 |
| ATOM | 19876 | CB | SER | N | 1428 | 79.495 | 94.710 | 48.941 | 1.00 | 102.03 | 6 |
| ATOM | 19877 | OG | SER | N | 1428 | 80.031 | 93.772 | 49.861 | 1.00 | 102.63 | 8 |
| ATOM | 19878 | C | SER | N | 1428 | 80.199 | 93.287 | 47.040 | 1.00 | 57.86 | 6 |
| ATOM | 19879 | O | SER | N | 1428 | 81.183 | 92.757 | 46.548 | 1.00 | 58.67 | 8 |
| ATOM | 19880 | N | ALA | N | 1429 | 79.018 | 92.701 | 47.083 | 1.00 | 69.75 | 7 |
| ATOM | 19881 | CA | ALA | N | 1429 | 78.816 | 91.373 | 46.532 | 1.00 | 72.29 | 6 |
| ATOM | 19882 | CB | ALA | N | 1429 | 77.338 | 91.048 | 46.523 | 1.00 | 161.19 | 6 |
| ATOM | 19883 | C | ALA | N | 1429 | 79.371 | 91.283 | 45.114 | 1.00 | 73.78 | 6 |
| ATOM | 19884 | O | ALA | N | 1429 | 79.478 | 90.198 | 44.544 | 1.00 | 74.63 | 8 |
| ATOM | 19885 | N | LEU | N | 1430 | 79.715 | 92.428 | 44.541 | 1.00 | 93.94 | 7 |
| ATOM | 19886 | CA | LEU | N | 1430 | 80.247 | 92.463 | 43.190 | 1.00 | 94.88 | 6 |
| ATOM | 19887 | CB | LEU | N | 1430 | 79.630 | 93.633 | 42.423 | 1.00 | 76.85 | 6 |
| ATOM | 19888 | CG | LEU | N | 1430 | 80.235 | 94.093 | 41.093 | 1.00 | 78.01 | 6 |
| ATOM | 19889 | CD1 | LEU | N | 1430 | 80.461 | 92.928 | 40.143 | 1.00 | 77.71 | 6 |
| ATOM | 19890 | CD2 | LEU | N | 1430 | 79.289 | 95.117 | 40.481 | 1.00 | 78.72 | 6 |
| ATOM | 19891 | C | LEU | N | 1430 | 81.761 | 92.581 | 43.182 | 1.00 | 95.68 | 6 |
| ATOM | 19892 | O | LEU | N | 1430 | 82.409 | 92.238 | 42.199 | 1.00 | 96.02 | 8 |
| ATOM | 19893 | N | SER | N | 1431 | 82.332 | 93.050 | 44.280 | 1.00 | 119.83 | 7 |
| ATOM | 19894 | CA | SER | N | 1431 | 83.771 | 93.214 | 44.336 | 1.00 | 121.26 | 6 |
| ATOM | 19895 | CB | SER | N | 1431 | 84.111 | 94.698 | 44.512 | 1.00 | 192.28 | 6 |
| ATOM | 19896 | OG | SER | N | 1431 | 85.506 | 94.902 | 44.669 | 1.00 | 193.66 | 8 |
| ATOM | 19897 | C | SER | N | 1431 | 84.430 | 92.412 | 45.439 | 1.00 | 120.88 | 6 |
| ATOM | 19898 | O | SER | N | 1431 | 84.908 | 92.999 | 46.408 | 1.00 | 122.18 | 8 |
| ATOM | 19899 | N | THR | N | 1432 | 84.468 | 91.084 | 45.311 | 1.00 | 75.41 | 7 |
| ATOM | 19900 | CA | THR | N | 1432 | 85.117 | 90.293 | 46.361 | 1.00 | 74.58 | 6 |
| ATOM | 19901 | CB | THR | N | 1432 | 84.540 | 90.645 | 47.748 | 1.00 | 198.41 | 6 |
| ATOM | 19902 | OG1 | THR | N | 1432 | 85.351 | 90.058 | 48.776 | 1.00 | 198.05 | 8 |
| ATOM | 19903 | CG2 | THR | N | 1432 | 83.121 | 90.117 | 47.875 | 1.00 | 199.11 | 6 |
| ATOM | 19904 | C | THR | N | 1432 | 85.172 | 88.766 | 46.326 | 1.00 | 72.80 | 6 |
| ATOM | 19905 | O | THR | N | 1432 | 84.315 | 88.093 | 45.749 | 1.00 | 71.38 | 8 |
| ATOM | 19906 | N | LYS | N | 1433 | 86.211 | 88.273 | 47.003 | 1.00 | 65.86 | 7 |
| ATOM | 19907 | CA | LYS | N | 1433 | 86.534 | 86.868 | 47.242 | 1.00 | 64.07 | 6 |
| ATOM | 19908 | CB | LYS | N | 1433 | 85.510 | 86.287 | 48.236 | 1.00 | 208.87 | 6 |
| ATOM | 19909 | CG | LYS | N | 1433 | 84.057 | 86.430 | 47.797 | 1.00 | 208.87 | 6 |
| ATOM | 19910 | CD | LYS | N | 1433 | 83.064 | 86.186 | 48.918 | 1.00 | 208.87 | 6 |
| ATOM | 19911 | CE | LYS | N | 1433 | 81.651 | 86.472 | 48.428 | 1.00 | 208.87 | 6 |
| ATOM | 19912 | NZ | LYS | N | 1433 | 80.626 | 86.302 | 49.491 | 1.00 | 208.87 | 7 |
| ATOM | 19913 | C | LYS | N | 1433 | 86.759 | 85.861 | 46.116 | 1.00 | 61.69 | 6 |
| ATOM | 19914 | O | LYS | N | 1433 | 87.853 | 85.777 | 45.551 | 1.00 | 61.66 | 8 |
| ATOM | 19915 | N | SER | N | 1434 | 85.727 | 85.068 | 45.833 | 1.00 | 68.84 | 7 |
| ATOM | 19916 | CA | SER | N | 1434 | 85.784 | 84.027 | 44.818 | 1.00 | 64.91 | 6 |
| ATOM | 19917 | CB | SER | N | 1434 | 84.826 | 82.901 | 45.170 | 1.00 | 54.55 | 6 |
| ATOM | 19918 | OG | SER | N | 1434 | 84.692 | 82.041 | 44.056 | 1.00 | 56.40 | 8 |
| ATOM | 19919 | C | SER | N | 1434 | 85.501 | 84.467 | 43.394 | 1.00 | 60.94 | 6 |
| ATOM | 19920 | O | SER | N | 1434 | 84.435 | 85.003 | 43.091 | 1.00 | 59.69 | 8 |
| ATOM | 19921 | N | TRP | N | 1435 | 86.472 | 84.200 | 42.528 | 1.00 | 69.82 | 7 |
| ATOM | 19922 | CA | TRP | N | 1435 | 86.400 | 84.531 | 41.111 | 1.00 | 65.35 | 6 |
| ATOM | 19923 | CB | TRP | N | 1435 | 87.620 | 83.914 | 40.392 | 1.00 | 42.09 | 6 |
| ATOM | 19924 | CG | TRP | N | 1435 | 87.366 | 82.582 | 39.702 | 1.00 | 36.85 | 6 |
| ATOM | 19925 | CD2 | TRP | N | 1435 | 87.664 | 81.275 | 40.205 | 1.00 | 34.58 | 6 |
| ATOM | 19926 | CE2 | TRP | N | 1435 | 87.179 | 80.341 | 39.261 | 1.00 | 33.86 | 6 |
| ATOM | 19927 | CE3 | TRP | N | 1435 | 88.291 | 80.800 | 41.359 | 1.00 | 33.72 | 6 |
| ATOM | 19928 | CD1 | TRP | N | 1435 | 86.738 | 82.391 | 38.500 | 1.00 | 35.21 | 6 |
| ATOM | 19929 | NE1 | TRP | N | 1435 | 86.621 | 81.050 | 38.231 | 1.00 | 34.18 | 7 |
| ATOM | 19930 | CZ2 | TRP | N | 1435 | 87.298 | 78.963 | 39.442 | 1.00 | 33.23 | 6 |
| ATOM | 19931 | CZ3 | TRP | N | 1435 | 88.412 | 79.425 | 41.537 | 1.00 | 33.70 | 6 |
| ATOM | 19932 | CH2 | TRP | N | 1435 | 87.916 | 78.527 | 40.583 | 1.00 | 33.93 | 6 |
| ATOM | 19933 | C | TRP | N | 1435 | 85.100 | 83.980 | 40.526 | 1.00 | 64.27 | 6 |
| ATOM | 19934 | O | TRP | N | 1435 | 84.442 | 84.614 | 39.692 | 1.00 | 64.33 | 8 |
| ATOM | 19935 | N | LEU | N | 1436 | 84.753 | 82.789 | 41.006 | 1.00 | 80.10 | 7 |
| ATOM | 19936 | CA | LEU | N | 1436 | 83.582 | 82.028 | 40.588 | 1.00 | 77.84 | 6 |
| ATOM | 19937 | CB | LEU | N | 1436 | 83.444 | 80.821 | 41.515 | 1.00 | 36.92 | 6 |
| ATOM | 19938 | CG | LEU | N | 1436 | 82.841 | 79.556 | 40.916 | 1.00 | 34.67 | 6 |
| ATOM | 19939 | CD1 | LEU | N | 1436 | 83.689 | 79.106 | 39.737 | 1.00 | 33.49 | 6 |
| ATOM | 19940 | CD2 | LEU | N | 1436 | 82.754 | 78.461 | 41.976 | 1.00 | 34.28 | 6 |
| ATOM | 19941 | C | LEU | N | 1436 | 82.247 | 82.785 | 40.507 | 1.00 | 77.74 | 6 |
| ATOM | 19942 | O | LEU | N | 1436 | 81.628 | 82.842 | 39.446 | 1.00 | 78.00 | 8 |
| ATOM | 19943 | N | SER | N | 1437 | 81.811 | 83.355 | 41.625 | 1.00 | 46.06 | 7 |
| ATOM | 19944 | CA | SER | N | 1437 | 80.555 | 84.095 | 41.695 | 1.00 | 44.46 | 6 |
| ATOM | 19945 | CB | SER | N | 1437 | 80.059 | 84.094 | 43.131 | 1.00 | 56.11 | 6 |
| ATOM | 19946 | OG | SER | N | 1437 | 81.126 | 84.336 | 44.030 | 1.00 | 56.82 | 8 |
| ATOM | 19947 | C | SER | N | 1437 | 80.602 | 85.531 | 41.181 | 1.00 | 43.34 | 6 |
| ATOM | 19948 | O | SER | N | 1437 | 79.568 | 86.105 | 40.866 | 1.00 | 42.72 | 8 |
| ATOM | 19949 | N | ALA | N | 1438 | 81.793 | 86.121 | 41.121 | 1.00 | 25.38 | 7 |
| ATOM | 19950 | CA | ALA | N | 1438 | 81.939 | 87.483 | 40.616 | 1.00 | 24.36 | 6 |
| ATOM | 19951 | CB | ALA | N | 1438 | 83.262 | 88.062 | 41.010 | 1.00 | 13.87 | 6 |
| ATOM | 19952 | C | ALA | N | 1438 | 81.885 | 87.363 | 39.117 | 1.00 | 24.32 | 6 |
| ATOM | 19953 | O | ALA | N | 1438 | 81.865 | 88.371 | 38.406 | 1.00 | 24.34 | 8 |
| ATOM | 19954 | N | ALA | N | 1439 | 81.896 | 86.106 | 38.663 | 1.00 | 31.44 | 7 |
| ATOM | 19955 | CA | ALA | N | 1439 | 81.842 | 85.716 | 37.247 | 1.00 | 32.45 | 6 |
| ATOM | 19956 | CB | ALA | N | 1439 | 82.396 | 84.309 | 37.086 | 1.00 | 13.87 | 6 |
| ATOM | 19957 | C | ALA | N | 1439 | 80.391 | 85.738 | 36.791 | 1.00 | 33.49 | 6 |
| ATOM | 19958 | O | ALA | N | 1439 | 80.067 | 86.032 | 35.635 | 1.00 | 33.10 | 8 |
| ATOM | 19959 | N | SER | N | 1440 | 79.528 | 85.387 | 37.732 | 1.00 | 37.46 | 7 |
| ATOM | 19960 | CA | SER | N | 1440 | 78.112 | 85.352 | 37.490 | 1.00 | 36.77 | 6 |
| ATOM | 19961 | CB | SER | N | 1440 | 77.360 | 85.090 | 38.795 | 1.00 | 58.28 | 6 |
| ATOM | 19962 | OG | SER | N | 1440 | 77.856 | 83.943 | 39.465 | 1.00 | 58.96 | 8 |
| ATOM | 19963 | C | SER | N | 1440 | 77.824 | 86.742 | 37.009 | 1.00 | 36.24 | 6 |
| ATOM | 19964 | O | SER | N | 1440 | 76.954 | 86.953 | 36.162 | 1.00 | 35.69 | 8 |
| ATOM | 19965 | N | PHE | N | 1441 | 78.589 | 87.690 | 37.536 | 1.00 | 17.74 | 7 |
| ATOM | 19966 | CA | PHE | N | 1441 | 78.373 | 89.074 | 37.176 | 1.00 | 19.96 | 6 |
| ATOM | 19967 | CB | PHE | N | 1441 | 79.172 | 89.992 | 38.093 | 1.00 | 30.81 | 6 |
| ATOM | 19968 | CG | PHE | N | 1441 | 78.640 | 90.034 | 39.499 | 1.00 | 30.88 | 6 |
| ATOM | 19969 | CD1 | PHE | N | 1441 | 79.099 | 89.143 | 40.461 | 1.00 | 30.90 | 6 |
| ATOM | 19970 | CD2 | PHE | N | 1441 | 77.650 | 90.939 | 39.854 | 1.00 | 29.97 | 6 |
| ATOM | 19971 | CE1 | PHE | N | 1441 | 78.574 | 89.160 | 41.754 | 1.00 | 29.52 | 6 |
| ATOM | 19972 | CE2 | PHE | N | 1441 | 77.124 | 90.955 | 41.144 | 1.00 | 29.34 | 6 |
| ATOM | 19973 | CZ | PHE | N | 1441 | 77.585 | 90.068 | 42.091 | 1.00 | 28.33 | 6 |
| ATOM | 19974 | C | PHE | N | 1441 | 78.614 | 89.420 | 35.719 | 1.00 | 21.74 | 6 |
| ATOM | 19975 | O | PHE | N | 1441 | 79.110 | 88.592 | 34.947 | 1.00 | 21.70 | 8 |
| ATOM | 19976 | N | GLN | N | 1442 | 78.219 | 90.644 | 35.357 | 1.00 | 67.06 | 7 |
| ATOM | 19977 | CA | GLN | N | 1442 | 78.336 | 91.184 | 34.000 | 1.00 | 69.94 | 6 |
| ATOM | 19978 | CB | GLN | N | 1442 | 77.372 | 92.371 | 33.846 | 1.00 | 109.63 | 6 |
| ATOM | 19979 | CG | GLN | N | 1442 | 77.268 | 92.989 | 32.449 | 1.00 | 113.01 | 6 |
| ATOM | 19980 | CD | GLN | N | 1442 | 76.480 | 92.137 | 31.463 | 1.00 | 115.85 | 6 |
| ATOM | 19981 | OE1 | GLN | N | 1442 | 75.370 | 91.689 | 31.754 | 1.00 | 116.49 | 8 |
| ATOM | 19982 | NE2 | GLN | N | 1442 | 77.049 | 91.923 | 30.280 | 1.00 | 117.78 | 7 |
| ATOM | 19983 | C | GLN | N | 1442 | 79.775 | 91.638 | 33.743 | 1.00 | 71.00 | 6 |
| ATOM | 19984 | O | GLN | N | 1442 | 80.044 | 92.832 | 33.597 | 1.00 | 71.29 | 8 |
| ATOM | 19985 | N | ASN | N | 1443 | 80.690 | 90.674 | 33.683 | 1.00 | 72.82 | 7 |
| ATOM | 19986 | CA | ASN | N | 1443 | 82.107 | 90.941 | 33.468 | 1.00 | 72.00 | 6 |
| ATOM | 19987 | CB | ASN | N | 1443 | 82.568 | 92.077 | 34.385 | 1.00 | 106.27 | 6 |
| ATOM | 19988 | CG | ASN | N | 1443 | 82.405 | 91.741 | 35.862 | 1.00 | 108.12 | 6 |
| ATOM | 19989 | OD1 | ASN | N | 1443 | 81.469 | 91.040 | 36.254 | 1.00 | 107.31 | 8 |
| ATOM | 19990 | ND2 | ASN | N | 1443 | 83.312 | 92.254 | 36.691 | 1.00 | 110.30 | 7 |
| ATOM | 19991 | C | ASN | N | 1443 | 82.875 | 89.668 | 33.801 | 1.00 | 71.20 | 6 |
| ATOM | 19992 | O | ASN | N | 1443 | 83.833 | 89.690 | 34.570 | 1.00 | 71.75 | 8 |
| ATOM | 19993 | N | THR | N | 1444 | 82.448 | 88.554 | 33.219 | 1.00 | 62.95 | 7 |
| ATOM | 19994 | CA | THR | N | 1444 | 83.089 | 87.267 | 33.472 | 1.00 | 62.82 | 6 |
| ATOM | 19995 | CB | THR | N | 1444 | 82.246 | 86.127 | 32.894 | 1.00 | 61.77 | 6 |
| ATOM | 19996 | OG1 | THR | N | 1444 | 80.933 | 86.174 | 33.472 | 1.00 | 62.25 | 8 |
| ATOM | 19997 | CG2 | THR | N | 1444 | 82.889 | 84.785 | 33.204 | 1.00 | 62.01 | 6 |
| ATOM | 19998 | C | THR | N | 1444 | 84.510 | 87.176 | 32.912 | 1.00 | 63.05 | 6 |
| ATOM | 19999 | O | THR | N | 1444 | 85.425 | 86.731 | 33.607 | 1.00 | 62.69 | 8 |
| ATOM | 20000 | N | THR | N | 1445 | 84.687 | 87.589 | 31.655 | 1.00 | 31.70 | 7 |
| ATOM | 20001 | CA | THR | N | 1445 | 85.999 | 87.571 | 31.012 | 1.00 | 30.06 | 6 |
| ATOM | 20002 | CB | THR | N | 1445 | 86.068 | 88.526 | 29.809 | 1.00 | 61.07 | 6 |
| ATOM | 20003 | OG1 | THR | N | 1445 | 85.025 | 88.222 | 28.876 | 1.00 | 61.05 | 8 |
| ATOM | 20004 | CG2 | THR | N | 1445 | 87.398 | 88.391 | 29.123 | 1.00 | 61.55 | 6 |
| ATOM | 20005 | C | THR | N | 1445 | 86.978 | 88.073 | 32.039 | 1.00 | 29.81 | 6 |
| ATOM | 20006 | O | THR | N | 1445 | 87.773 | 87.322 | 32.567 | 1.00 | 28.84 | 8 |
| ATOM | 20007 | N | HIS | N | 1446 | 86.881 | 89.359 | 32.336 | 1.00 | 53.13 | 7 |
| ATOM | 20008 | CA | HIS | N | 1446 | 87.756 | 89.997 | 33.304 | 1.00 | 54.87 | 6 |
| ATOM | 20009 | CB | HIS | N | 1446 | 87.204 | 91.375 | 33.678 | 1.00 | 67.65 | 6 |
| ATOM | 20010 | CG | HIS | N | 1446 | 88.143 | 92.198 | 34.501 | 1.00 | 69.25 | 6 |
| ATOM | 20011 | CD2 | HIS | N | 1446 | 89.170 | 91.841 | 35.311 | 1.00 | 69.60 | 6 |
| ATOM | 20012 | ND1 | HIS | N | 1446 | 88.062 | 93.572 | 34.567 | 1.00 | 69.66 | 7 |
| ATOM | 20013 | CE1 | HIS | N | 1446 | 88.997 | 94.027 | 35.383 | 1.00 | 70.54 | 6 |
| ATOM | 20014 | NE2 | HIS | N | 1446 | 89.683 | 92.997 | 35.848 | 1.00 | 70.59 | 7 |
| ATOM | 20015 | C | HIS | N | 1446 | 87.937 | 89.139 | 34.550 | 1.00 | 55.28 | 6 |
| ATOM | 20016 | O | HIS | N | 1446 | 89.032 | 88.628 | 34.785 | 1.00 | 55.67 | 8 |
| ATOM | 20017 | N | VAL | N | 1447 | 86.880 | 88.978 | 35.343 | 1.00 | 27.83 | 7 |
| ATOM | 20018 | CA | VAL | N | 1447 | 86.973 | 88.166 | 36.554 | 1.00 | 28.47 | 6 |
| ATOM | 20019 | CB | VAL | N | 1447 | 85.596 | 87.736 | 37.066 | 1.00 | 68.41 | 6 |
| ATOM | 20020 | CG1 | VAL | N | 1447 | 85.758 | 86.848 | 38.295 | 1.00 | 68.91 | 6 |
| ATOM | 20021 | CG2 | VAL | N | 1447 | 84.760 | 88.950 | 37.389 | 1.00 | 70.01 | 6 |
| ATOM | 20022 | C | VAL | N | 1447 | 87.736 | 86.894 | 36.251 | 1.00 | 28.79 | 6 |
| ATOM | 20023 | O | VAL | N | 1447 | 88.776 | 86.616 | 36.847 | 1.00 | 28.26 | 8 |
| ATOM | 20024 | N | LEU | N | 1448 | 87.198 | 86.132 | 35.303 | 1.00 | 83.43 | 7 |
| ATOM | 20025 | CA | LEU | N | 1448 | 87.770 | 84.862 | 34.883 | 1.00 | 83.81 | 6 |
| ATOM | 20026 | CB | LEU | N | 1448 | 86.862 | 84.220 | 33.825 | 1.00 | 54.44 | 6 |
| ATOM | 20027 | CG | LEU | N | 1448 | 86.245 | 82.852 | 34.173 | 1.00 | 54.53 | 6 |
| ATOM | 20028 | CD1 | LEU | N | 1448 | 85.696 | 82.867 | 35.588 | 1.00 | 54.24 | 6 |
| ATOM | 20029 | CD2 | LEU | N | 1448 | 85.151 | 82.498 | 33.167 | 1.00 | 53.83 | 6 |
| ATOM | 20030 | C | LEU | N | 1448 | 89.195 | 85.013 | 34.363 | 1.00 | 83.93 | 6 |
| ATOM | 20031 | O | LEU | N | 1448 | 90.049 | 84.172 | 34.625 | 1.00 | 84.69 | 8 |
| ATOM | 20032 | N | THR | N | 1449 | 89.453 | 86.090 | 33.632 | 1.00 | 40.52 | 7 |
| ATOM | 20033 | CA | THR | N | 1449 | 90.787 | 86.346 | 33.102 | 1.00 | 40.49 | 6 |
| ATOM | 20034 | CB | THR | N | 1449 | 90.837 | 87.684 | 32.318 | 1.00 | 41.10 | 6 |
| ATOM | 20035 | OG1 | THR | N | 1449 | 90.378 | 87.478 | 30.976 | 1.00 | 39.05 | 8 |
| ATOM | 20036 | CG2 | THR | N | 1449 | 92.257 | 88.246 | 32.293 | 1.00 | 40.51 | 6 |
| ATOM | 20037 | C | THR | N | 1449 | 91.760 | 86.435 | 34.270 | 1.00 | 41.44 | 6 |
| ATOM | 20038 | O | THR | N | 1449 | 92.687 | 85.638 | 34.384 | 1.00 | 40.81 | 8 |
| ATOM | 20039 | N | GLU | N | 1450 | 91.535 | 87.422 | 35.129 | 1.00 | 54.30 | 7 |
| ATOM | 20040 | CA | GLU | N | 1450 | 92.373 | 87.638 | 36.296 | 1.00 | 56.31 | 6 |
| ATOM | 20041 | CB | GLU | N | 1450 | 91.700 | 88.641 | 37.244 | 1.00 | 175.21 | 6 |
| ATOM | 20042 | CG | GLU | N | 1450 | 92.337 | 88.764 | 38.634 | 1.00 | 178.88 | 6 |
| ATOM | 20043 | CD | GLU | N | 1450 | 93.730 | 89.372 | 38.611 | 1.00 | 182.13 | 6 |
| ATOM | 20044 | OE1 | GLU | N | 1450 | 93.884 | 90.494 | 38.081 | 1.00 | 184.88 | 8 |
| ATOM | 20045 | OE2 | GLU | N | 1450 | 94.671 | 88.734 | 39.132 | 1.00 | 183.57 | 8 |
| ATOM | 20046 | C | GLU | N | 1450 | 92.624 | 86.328 | 37.025 | 1.00 | 56.59 | 6 |
| ATOM | 20047 | O | GLU | N | 1450 | 93.732 | 85.792 | 36.999 | 1.00 | 56.22 | 8 |
| ATOM | 20048 | N | ALA | N | 1451 | 91.583 | 85.817 | 37.671 | 1.00 | 49.67 | 7 |
| ATOM | 20049 | CA | ALA | N | 1451 | 91.684 | 84.581 | 38.426 | 1.00 | 49.72 | 6 |
| ATOM | 20050 | CB | ALA | N | 1451 | 90.340 | 83.899 | 38.491 | 1.00 | 29.23 | 6 |
| ATOM | 20051 | C | ALA | N | 1451 | 92.702 | 83.666 | 37.785 | 1.00 | 50.05 | 6 |
| ATOM | 20052 | O | ALA | N | 1451 | 93.592 | 83.161 | 38.452 | 1.00 | 51.38 | 8 |
| ATOM | 20053 | N | ALA | N | 1452 | 92.571 | 83.464 | 36.481 | 1.00 | 84.61 | 7 |
| ATOM | 20054 | CA | ALA | N | 1452 | 93.492 | 82.606 | 35.754 | 1.00 | 84.25 | 6 |
| ATOM | 20055 | CB | ALA | N | 1452 | 93.231 | 82.708 | 34.260 | 1.00 | 114.98 | 6 |
| ATOM | 20056 | C | ALA | N | 1452 | 94.925 | 83.012 | 36.068 | 1.00 | 83.64 | 6 |
| ATOM | 20057 | O | ALA | N | 1452 | 95.697 | 82.212 | 36.593 | 1.00 | 84.27 | 8 |
| ATOM | 20058 | N | ILE | N | 1453 | 95.273 | 84.255 | 35.742 | 1.00 | 48.42 | 7 |
| ATOM | 20059 | CA | ILE | N | 1453 | 96.611 | 84.778 | 36.002 | 1.00 | 47.40 | 6 |
| ATOM | 20060 | CB | ILE | N | 1453 | 96.652 | 86.332 | 35.894 | 1.00 | 56.02 | 6 |
| ATOM | 20061 | CG2 | ILE | N | 1453 | 97.816 | 86.885 | 36.664 | 1.00 | 56.14 | 6 |
| ATOM | 20062 | CG1 | ILE | N | 1453 | 96.776 | 86.769 | 34.434 | 1.00 | 56.58 | 6 |
| ATOM | 20063 | CD1 | ILE | N | 1453 | 95.528 | 86.576 | 33.607 | 1.00 | 58.36 | 6 |
| ATOM | 20064 | C | ILE | N | 1453 | 96.996 | 84.361 | 37.413 | 1.00 | 46.66 | 6 |
| ATOM | 20065 | O | ILE | N | 1453 | 97.756 | 83.412 | 37.593 | 1.00 | 47.03 | 8 |
| ATOM | 20066 | N | ALA | N | 1454 | 96.455 | 85.051 | 38.414 | 1.00 | 37.06 | 7 |
| ATOM | 20067 | CA | ALA | N | 1454 | 96.768 | 84.727 | 39.800 | 1.00 | 37.18 | 6 |
| ATOM | 20068 | CB | ALA | N | 1454 | 95.939 | 85.585 | 40.723 | 1.00 | 26.87 | 6 |
| ATOM | 20069 | C | ALA | N | 1454 | 96.521 | 83.243 | 40.089 | 1.00 | 37.73 | 6 |
| ATOM | 20070 | O | ALA | N | 1454 | 96.110 | 82.492 | 39.205 | 1.00 | 37.29 | 8 |
| ATOM | 20071 | N | GLY | N | 1455 | 96.781 | 82.818 | 41.322 | 1.00 | 69.50 | 7 |
| ATOM | 20072 | CA | GLY | N | 1455 | 96.572 | 81.423 | 41.669 | 1.00 | 70.33 | 6 |
| ATOM | 20073 | C | GLY | N | 1455 | 95.087 | 81.159 | 41.721 | 1.00 | 71.07 | 6 |
| ATOM | 20074 | O | GLY | N | 1455 | 94.533 | 80.472 | 40.864 | 1.00 | 70.17 | 8 |
| ATOM | 20075 | N | LYS | N | 1456 | 94.453 | 81.719 | 42.746 | 1.00 | 94.06 | 7 |
| ATOM | 20076 | CA | LYS | N | 1456 | 93.018 | 81.608 | 42.961 | 1.00 | 94.89 | 6 |
| ATOM | 20077 | CB | LYS | N | 1456 | 92.289 | 82.055 | 41.697 | 1.00 | 81.13 | 6 |
| ATOM | 20078 | CG | LYS | N | 1456 | 92.501 | 83.527 | 41.368 | 1.00 | 82.00 | 6 |
| ATOM | 20079 | CD | LYS | N | 1456 | 91.904 | 84.414 | 42.460 | 1.00 | 83.34 | 6 |
| ATOM | 20080 | CE | LYS | N | 1456 | 91.744 | 85.859 | 42.012 | 1.00 | 84.06 | 6 |
| ATOM | 20081 | NZ | LYS | N | 1456 | 90.861 | 86.609 | 42.948 | 1.00 | 85.20 | 7 |
| ATOM | 20082 | C | LYS | N | 1456 | 92.524 | 80.234 | 43.389 | 1.00 | 94.74 | 6 |
| ATOM | 20083 | O | LYS | N | 1456 | 93.005 | 79.212 | 42.915 | 1.00 | 95.13 | 8 |
| ATOM | 20084 | N | LYS | N | 1457 | 91.565 | 80.228 | 44.307 | 1.00 | 94.29 | 7 |
| ATOM | 20085 | CA | LYS | N | 1457 | 90.960 | 78.996 | 44.806 | 1.00 | 95.55 | 6 |
| ATOM | 20086 | CB | LYS | N | 1457 | 91.834 | 78.321 | 45.860 | 1.00 | 32.50 | 6 |
| ATOM | 20087 | CG | LYS | N | 1457 | 93.304 | 78.176 | 45.530 | 1.00 | 32.29 | 6 |
| ATOM | 20088 | CD | LYS | N | 1457 | 93.971 | 77.148 | 46.469 | 1.00 | 31.26 | 6 |
| ATOM | 20089 | CE | LYS | N | 1457 | 93.641 | 77.416 | 47.949 | 1.00 | 31.48 | 6 |
| ATOM | 20090 | NZ | LYS | N | 1457 | 93.455 | 76.177 | 48.780 | 1.00 | 29.54 | 7 |
| ATOM | 20091 | C | LYS | N | 1457 | 89.647 | 79.382 | 45.470 | 1.00 | 97.43 | 6 |
| ATOM | 20092 | O | LYS | N | 1457 | 89.634 | 79.723 | 46.652 | 1.00 | 98.45 | 8 |
| ATOM | 20093 | N | ASP | N | 1458 | 88.549 | 79.318 | 44.720 | 1.00 | 52.00 | 7 |
| ATOM | 20094 | CA | ASP | N | 1458 | 87.233 | 79.687 | 45.236 | 1.00 | 52.36 | 6 |
| ATOM | 20095 | CB | ASP | N | 1458 | 86.137 | 78.958 | 44.464 | 1.00 | 130.17 | 6 |
| ATOM | 20096 | CG | ASP | N | 1458 | 84.754 | 79.431 | 44.847 | 1.00 | 132.81 | 6 |
| ATOM | 20097 | OD1 | ASP | N | 1458 | 84.466 | 79.507 | 46.056 | 1.00 | 134.02 | 8 |
| ATOM | 20098 | OD2 | ASP | N | 1458 | 83.949 | 79.724 | 43.945 | 1.00 | 134.12 | 8 |
| ATOM | 20099 | C | ASP | N | 1458 | 87.053 | 79.411 | 46.721 | 1.00 | 52.13 | 6 |
| ATOM | 20100 | O | ASP | N | 1458 | 86.552 | 80.255 | 47.457 | 1.00 | 52.31 | 8 |
| ATOM | 20101 | N | GLU | N | 1459 | 87.458 | 78.219 | 47.144 | 1.00 | 69.54 | 7 |
| ATOM | 20102 | CA | GLU | N | 1459 | 87.356 | 77.785 | 48.532 | 1.00 | 69.25 | 6 |
| ATOM | 20103 | CB | GLU | N | 1459 | 87.898 | 78.860 | 49.476 | 1.00 | 120.05 | 6 |
| ATOM | 20104 | CG | GLU | N | 1459 | 88.416 | 78.297 | 50.788 | 1.00 | 122.42 | 6 |
| ATOM | 20105 | CD | GLU | N | 1459 | 89.429 | 77.178 | 50.585 | 1.00 | 123.16 | 6 |
| ATOM | 20106 | OE1 | GLU | N | 1459 | 90.453 | 77.415 | 49.909 | 1.00 | 123.14 | 8 |
| ATOM | 20107 | OE2 | GLU | N | 1459 | 89.201 | 76.062 | 51.103 | 1.00 | 122.96 | 8 |
| ATOM | 20108 | C | GLU | N | 1459 | 85.906 | 77.464 | 48.848 | 1.00 | 67.80 | 6 |
| ATOM | 20109 | O | GLU | N | 1459 | 85.600 | 76.769 | 49.810 | 1.00 | 66.51 | 8 |
| ATOM | 20110 | N | LEU | N | 1460 | 85.023 | 77.979 | 48.005 | 1.00 | 57.98 | 7 |
| ATOM | 20111 | CA | LEU | N | 1460 | 83.590 | 77.755 | 48.104 | 1.00 | 57.91 | 6 |
| ATOM | 20112 | CB | LEU | N | 1460 | 83.305 | 76.293 | 47.742 | 1.00 | 28.57 | 6 |
| ATOM | 20113 | CG | LEU | N | 1460 | 84.031 | 75.902 | 46.436 | 1.00 | 27.18 | 6 |
| ATOM | 20114 | CD1 | LEU | N | 1460 | 83.756 | 74.448 | 46.084 | 1.00 | 26.65 | 6 |
| ATOM | 20115 | CD2 | LEU | N | 1460 | 83.593 | 76.803 | 45.293 | 1.00 | 25.81 | 6 |
| ATOM | 20116 | C | LEU | N | 1460 | 82.929 | 78.150 | 49.428 | 1.00 | 58.67 | 6 |
| ATOM | 20117 | O | LEU | N | 1460 | 82.386 | 77.313 | 50.153 | 1.00 | 59.22 | 8 |
| ATOM | 20118 | N | ILE | N | 1461 | 82.966 | 79.451 | 49.712 | 1.00 | 70.79 | 7 |
| ATOM | 20119 | CA | ILE | N | 1461 | 82.379 | 80.025 | 50.920 | 1.00 | 71.83 | 6 |
| ATOM | 20120 | CB | ILE | N | 1461 | 83.234 | 81.173 | 51.457 | 1.00 | 81.21 | 6 |
| ATOM | 20121 | CG2 | ILE | N | 1461 | 82.578 | 81.771 | 52.683 | 1.00 | 81.21 | 6 |
| ATOM | 20122 | CG1 | ILE | N | 1461 | 84.636 | 80.668 | 51.778 | 1.00 | 81.91 | 6 |
| ATOM | 20123 | CD1 | ILE | N | 1461 | 85.580 | 81.754 | 52.247 | 1.00 | 82.75 | 6 |
| ATOM | 20124 | C | ILE | N | 1461 | 81.010 | 80.609 | 50.597 | 1.00 | 72.68 | 6 |
| ATOM | 20125 | O | ILE | N | 1461 | 80.031 | 80.395 | 51.317 | 1.00 | 72.65 | 8 |
| ATOM | 20126 | N | GLY | N | 1462 | 80.964 | 81.370 | 49.513 | 1.00 | 132.81 | 7 |
| ATOM | 20127 | CA | GLY | N | 1462 | 79.724 | 81.987 | 49.094 | 1.00 | 133.02 | 6 |
| ATOM | 20128 | C | GLY | N | 1462 | 78.733 | 80.982 | 48.546 | 1.00 | 133.31 | 6 |
| ATOM | 20129 | O | GLY | N | 1462 | 79.033 | 80.240 | 47.611 | 1.00 | 133.17 | 8 |
| ATOM | 20130 | N | LEU | N | 1463 | 77.545 | 80.963 | 49.140 | 1.00 | 61.88 | 7 |
| ATOM | 20131 | CA | LEU | N | 1463 | 76.484 | 80.063 | 48.726 | 1.00 | 60.60 | 6 |
| ATOM | 20132 | CB | LEU | N | 1463 | 75.191 | 80.451 | 49.443 | 1.00 | 50.32 | 6 |
| ATOM | 20133 | CG | LEU | N | 1463 | 75.206 | 80.335 | 50.968 | 1.00 | 51.25 | 6 |
| ATOM | 20134 | CD1 | LEU | N | 1463 | 73.977 | 81.009 | 51.565 | 1.00 | 50.41 | 6 |
| ATOM | 20135 | CD2 | LEU | N | 1463 | 75.260 | 78.871 | 51.355 | 1.00 | 51.51 | 6 |
| ATOM | 20136 | C | LEU | N | 1463 | 76.270 | 80.080 | 47.206 | 1.00 | 59.77 | 6 |
| ATOM | 20137 | O | LEU | N | 1463 | 76.499 | 79.078 | 46.527 | 1.00 | 60.49 | 8 |
| ATOM | 20138 | N | LYS | N | 1464 | 75.846 | 81.220 | 46.669 | 1.00 | 72.25 | 7 |
| ATOM | 20139 | CA | LYS | N | 1464 | 75.591 | 81.314 | 45.238 | 1.00 | 71.28 | 6 |
| ATOM | 20140 | CB | LYS | N | 1464 | 75.068 | 82.697 | 44.862 | 1.00 | 79.80 | 6 |
| ATOM | 20141 | CG | LYS | N | 1464 | 73.658 | 82.925 | 45.369 | 1.00 | 81.73 | 6 |
| ATOM | 20142 | CD | LYS | N | 1464 | 72.926 | 84.014 | 44.604 | 1.00 | 82.90 | 6 |
| ATOM | 20143 | CE | LYS | N | 1464 | 71.541 | 84.200 | 45.190 | 1.00 | 83.03 | 6 |
| ATOM | 20144 | NZ | LYS | N | 1464 | 70.912 | 82.870 | 45.434 | 1.00 | 83.10 | 7 |
| ATOM | 20145 | C | LYS | N | 1464 | 76.776 | 80.961 | 44.373 | 1.00 | 70.10 | 6 |
| ATOM | 20146 | O | LYS | N | 1464 | 76.691 | 81.019 | 43.152 | 1.00 | 70.34 | 8 |
| ATOM | 20147 | N | GLU | N | 1465 | 77.886 | 80.602 | 44.996 | 1.00 | 71.22 | 7 |
| ATOM | 20148 | CA | GLU | N | 1465 | 79.055 | 80.206 | 44.234 | 1.00 | 70.09 | 6 |
| ATOM | 20149 | CB | GLU | N | 1465 | 80.324 | 80.535 | 45.004 | 1.00 | 129.79 | 6 |
| ATOM | 20150 | CG | GLU | N | 1465 | 80.288 | 81.883 | 45.670 | 1.00 | 133.90 | 6 |
| ATOM | 20151 | CD | GLU | N | 1465 | 81.538 | 82.151 | 46.457 | 1.00 | 136.35 | 6 |
| ATOM | 20152 | OE1 | GLU | N | 1465 | 81.920 | 81.284 | 47.269 | 1.00 | 137.37 | 8 |
| ATOM | 20153 | OE2 | GLU | N | 1465 | 82.137 | 83.228 | 46.267 | 1.00 | 138.46 | 8 |
| ATOM | 20154 | C | GLU | N | 1465 | 78.883 | 78.707 | 44.153 | 1.00 | 67.73 | 6 |
| ATOM | 20155 | O | GLU | N | 1465 | 78.694 | 78.123 | 43.087 | 1.00 | 68.58 | 8 |
| ATOM | 20156 | N | ASN | N | 1466 | 78.933 | 78.101 | 45.326 | 1.00 | 43.12 | 7 |
| ATOM | 20157 | CA | ASN | N | 1466 | 78.765 | 76.672 | 45.482 | 1.00 | 39.82 | 6 |
| ATOM | 20158 | CB | ASN | N | 1466 | 78.546 | 76.371 | 46.971 | 1.00 | 50.98 | 6 |
| ATOM | 20159 | CG | ASN | N | 1466 | 79.244 | 75.117 | 47.434 | 1.00 | 53.23 | 6 |
| ATOM | 20160 | OD1 | ASN | N | 1466 | 78.650 | 74.040 | 47.474 | 1.00 | 53.76 | 8 |
| ATOM | 20161 | ND2 | ASN | N | 1466 | 80.515 | 75.251 | 47.797 | 1.00 | 54.90 | 7 |
| ATOM | 20162 | C | ASN | N | 1466 | 77.527 | 76.307 | 44.662 | 1.00 | 37.12 | 6 |
| ATOM | 20163 | O | ASN | N | 1466 | 77.410 | 75.190 | 44.148 | 1.00 | 36.93 | 8 |
| ATOM | 20164 | N | VAL | N | 1467 | 76.593 | 77.247 | 44.543 | 1.00 | 24.65 | 7 |
| ATOM | 20165 | CA | VAL | N | 1467 | 75.407 | 76.952 | 43.761 | 1.00 | 21.85 | 6 |
| ATOM | 20166 | CB | VAL | N | 1467 | 74.289 | 78.074 | 43.883 | 1.00 | 15.60 | 6 |
| ATOM | 20167 | CG1 | VAL | N | 1467 | 73.494 | 78.226 | 42.570 | 1.00 | 13.99 | 6 |
| ATOM | 20168 | CG2 | VAL | N | 1467 | 73.302 | 77.693 | 44.989 | 1.00 | 14.47 | 6 |
| ATOM | 20169 | C | VAL | N | 1467 | 75.895 | 76.796 | 42.335 | 1.00 | 21.30 | 6 |
| ATOM | 20170 | O | VAL | N | 1467 | 75.772 | 75.730 | 41.759 | 1.00 | 20.21 | 8 |
| ATOM | 20171 | N | ILE | N | 1468 | 76.488 | 77.847 | 41.783 | 1.00 | 28.96 | 7 |
| ATOM | 20172 | CA | ILE | N | 1468 | 76.984 | 77.816 | 40.413 | 1.00 | 28.15 | 6 |
| ATOM | 20173 | CB | ILE | N | 1468 | 78.039 | 78.901 | 40.221 | 1.00 | 28.50 | 6 |
| ATOM | 20174 | CG2 | ILE | N | 1468 | 78.665 | 78.784 | 38.840 | 1.00 | 28.28 | 6 |
| ATOM | 20175 | CG1 | ILE | N | 1468 | 77.386 | 80.265 | 40.454 | 1.00 | 28.03 | 6 |
| ATOM | 20176 | CD1 | ILE | N | 1468 | 78.363 | 81.388 | 40.570 | 1.00 | 27.94 | 6 |
| ATOM | 20177 | C | ILE | N | 1468 | 77.546 | 76.447 | 40.020 | 1.00 | 28.16 | 6 |
| ATOM | 20178 | O | ILE | N | 1468 | 77.203 | 75.911 | 38.969 | 1.00 | 27.93 | 8 |
| ATOM | 20179 | N | LEU | N | 1469 | 78.400 | 75.879 | 40.866 | 1.00 | 41.09 | 7 |
| ATOM | 20180 | CA | LEU | N | 1469 | 78.958 | 74.557 | 40.588 | 1.00 | 41.71 | 6 |
| ATOM | 20181 | CB | LEU | N | 1469 | 80.166 | 74.254 | 41.478 | 1.00 | 40.28 | 6 |
| ATOM | 20182 | CG | LEU | N | 1469 | 81.322 | 75.226 | 41.701 | 1.00 | 40.00 | 6 |
| ATOM | 20183 | CD1 | LEU | N | 1469 | 82.281 | 74.557 | 42.666 | 1.00 | 39.89 | 6 |
| ATOM | 20184 | CD2 | LEU | N | 1469 | 82.032 | 75.575 | 40.407 | 1.00 | 39.68 | 6 |
| ATOM | 20185 | C | LEU | N | 1469 | 77.877 | 73.529 | 40.911 | 1.00 | 42.41 | 6 |
| ATOM | 20186 | O | LEU | N | 1469 | 78.174 | 72.357 | 41.100 | 1.00 | 43.44 | 8 |
| ATOM | 20187 | N | GLY | N | 1470 | 76.628 | 73.975 | 40.992 | 1.00 | 32.51 | 7 |
| ATOM | 20188 | CA | GLY | N | 1470 | 75.527 | 73.080 | 41.316 | 1.00 | 31.96 | 6 |
| ATOM | 20189 | C | GLY | N | 1470 | 75.752 | 72.287 | 42.594 | 1.00 | 31.97 | 6 |
| ATOM | 20190 | O | GLY | N | 1470 | 74.978 | 71.372 | 42.913 | 1.00 | 30.88 | 8 |
| ATOM | 20191 | N | ARG | N | 1471 | 76.801 | 72.653 | 43.332 | 1.00 | 71.36 | 7 |
| ATOM | 20192 | CA | ARG | N | 1471 | 77.159 | 71.956 | 44.561 | 1.00 | 72.29 | 6 |
| ATOM | 20193 | CB | ARG | N | 1471 | 78.442 | 72.535 | 45.166 | 1.00 | 73.54 | 6 |
| ATOM | 20194 | CG | ARG | N | 1471 | 79.732 | 71.877 | 44.668 | 1.00 | 77.78 | 6 |
| ATOM | 20195 | CD | ARG | N | 1471 | 80.914 | 72.219 | 45.583 | 1.00 | 81.38 | 6 |
| ATOM | 20196 | NE | ARG | N | 1471 | 80.775 | 71.640 | 46.923 | 1.00 | 85.00 | 7 |
| ATOM | 20197 | CZ | ARG | N | 1471 | 81.475 | 72.026 | 47.992 | 1.00 | 86.24 | 6 |
| ATOM | 20198 | NH1 | ARG | N | 1471 | 82.369 | 72.999 | 47.894 | 1.00 | 86.86 | 7 |
| ATOM | 20199 | NH2 | ARG | N | 1471 | 81.282 | 71.444 | 49.168 | 1.00 | 86.50 | 7 |
| ATOM | 20200 | C | ARG | N | 1471 | 76.082 | 71.871 | 45.629 | 1.00 | 70.82 | 6 |
| ATOM | 20201 | O | ARG | N | 1471 | 74.929 | 72.247 | 45.406 | 1.00 | 70.77 | 8 |
| ATOM | 20202 | N | LEU | N | 1472 | 76.490 | 71.377 | 46.794 | 1.00 | 48.63 | 7 |
| ATOM | 20203 | CA | LEU | N | 1472 | 75.598 | 71.150 | 47.920 | 1.00 | 45.59 | 6 |
| ATOM | 20204 | CB | LEU | N | 1472 | 75.887 | 69.763 | 48.483 | 1.00 | 33.60 | 6 |
| ATOM | 20205 | CG | LEU | N | 1472 | 75.373 | 69.335 | 49.852 | 1.00 | 32.93 | 6 |
| ATOM | 20206 | CD1 | LEU | N | 1472 | 73.873 | 69.393 | 49.905 | 1.00 | 33.13 | 6 |
| ATOM | 20207 | CD2 | LEU | N | 1472 | 75.852 | 67.924 | 50.115 | 1.00 | 33.31 | 6 |
| ATOM | 20208 | C | LEU | N | 1472 | 75.630 | 72.163 | 49.047 | 1.00 | 43.50 | 6 |
| ATOM | 20209 | O | LEU | N | 1472 | 76.224 | 71.901 | 50.086 | 1.00 | 42.73 | 8 |
| ATOM | 20210 | N | ILE | N | 1473 | 74.978 | 73.304 | 48.838 | 1.00 | 40.42 | 7 |
| ATOM | 20211 | CA | ILE | N | 1473 | 74.878 | 74.383 | 49.820 | 1.00 | 40.08 | 6 |
| ATOM | 20212 | CB | ILE | N | 1473 | 73.407 | 74.622 | 50.203 | 1.00 | 25.68 | 6 |
| ATOM | 20213 | CG2 | ILE | N | 1473 | 73.314 | 75.499 | 51.428 | 1.00 | 24.29 | 6 |
| ATOM | 20214 | CG1 | ILE | N | 1473 | 72.670 | 75.293 | 49.054 | 1.00 | 26.68 | 6 |
| ATOM | 20215 | CD1 | ILE | N | 1473 | 73.169 | 76.692 | 48.733 | 1.00 | 27.55 | 6 |
| ATOM | 20216 | C | ILE | N | 1473 | 75.653 | 74.208 | 51.128 | 1.00 | 41.31 | 6 |
| ATOM | 20217 | O | ILE | N | 1473 | 75.399 | 73.281 | 51.903 | 1.00 | 42.70 | 8 |
| ATOM | 20218 | N | PRO | N | 1474 | 76.609 | 75.105 | 51.401 | 1.00 | 63.72 | 7 |
| ATOM | 20219 | CD | PRO | N | 1474 | 77.206 | 76.163 | 50.569 | 1.00 | 14.38 | 6 |
| ATOM | 20220 | CA | PRO | N | 1474 | 77.336 | 74.937 | 52.660 | 1.00 | 63.88 | 6 |
| ATOM | 20221 | CB | PRO | N | 1474 | 78.427 | 75.990 | 52.583 | 1.00 | 13.87 | 6 |
| ATOM | 20222 | CG | PRO | N | 1474 | 77.891 | 77.001 | 51.597 | 1.00 | 13.87 | 6 |
| ATOM | 20223 | C | PRO | N | 1474 | 76.417 | 75.142 | 53.853 | 1.00 | 65.05 | 6 |
| ATOM | 20224 | O | PRO | N | 1474 | 76.455 | 74.372 | 54.797 | 1.00 | 65.63 | 8 |
| ATOM | 20225 | N | ALA | N | 1475 | 75.584 | 76.175 | 53.809 | 1.00 | 31.81 | 7 |
| ATOM | 20226 | CA | ALA | N | 1475 | 74.645 | 76.454 | 54.898 | 1.00 | 32.54 | 6 |
| ATOM | 20227 | CB | ALA | N | 1475 | 73.958 | 77.792 | 54.651 | 1.00 | 124.85 | 6 |
| ATOM | 20228 | C | ALA | N | 1475 | 73.589 | 75.348 | 55.065 | 1.00 | 31.96 | 6 |
| ATOM | 20229 | O | ALA | N | 1475 | 73.535 | 74.400 | 54.281 | 1.00 | 31.04 | 8 |
| ATOM | 20230 | N | GLY | N | 1476 | 72.755 | 75.474 | 56.093 | 1.00 | 41.35 | 7 |
| ATOM | 20231 | CA | GLY | N | 1476 | 71.713 | 74.488 | 56.337 | 1.00 | 42.52 | 6 |
| ATOM | 20232 | C | GLY | N | 1476 | 72.203 | 73.068 | 56.545 | 1.00 | 42.98 | 6 |
| ATOM | 20233 | O | GLY | N | 1476 | 73.299 | 72.861 | 57.057 | 1.00 | 42.28 | 8 |
| ATOM | 20234 | N | THR | N | 1477 | 71.397 | 72.081 | 56.165 | 1.00 | 41.50 | 7 |
| ATOM | 20235 | CA | THR | N | 1477 | 71.813 | 70.693 | 56.313 | 1.00 | 42.80 | 6 |
| ATOM | 20236 | CB | THR | N | 1477 | 70.900 | 69.719 | 55.538 | 1.00 | 44.03 | 6 |
| ATOM | 20237 | OG1 | THR | N | 1477 | 70.315 | 70.388 | 54.417 | 1.00 | 42.93 | 8 |
| ATOM | 20238 | CG2 | THR | N | 1477 | 69.821 | 69.182 | 56.428 | 1.00 | 44.34 | 6 |
| ATOM | 20239 | C | THR | N | 1477 | 73.220 | 70.529 | 55.761 | 1.00 | 44.91 | 6 |
| ATOM | 20240 | O | THR | N | 1477 | 73.857 | 69.502 | 55.977 | 1.00 | 45.66 | 8 |
| ATOM | 20241 | N | GLY | N | 1478 | 73.693 | 71.546 | 55.045 | 1.00 | 57.56 | 7 |
| ATOM | 20242 | CA | GLY | N | 1478 | 75.016 | 71.493 | 54.458 | 1.00 | 60.51 | 6 |
| ATOM | 20243 | C | GLY | N | 1478 | 76.146 | 71.867 | 55.396 | 1.00 | 63.35 | 6 |
| ATOM | 20244 | O | GLY | N | 1478 | 77.295 | 71.519 | 55.134 | 1.00 | 64.75 | 8 |
| ATOM | 20245 | N | SER | N | 1479 | 75.842 | 72.573 | 56.482 | 1.00 | 38.88 | 7 |
| ATOM | 20246 | CA | SER | N | 1479 | 76.881 | 72.984 | 57.426 | 1.00 | 40.64 | 6 |
| ATOM | 20247 | CB | SER | N | 1479 | 76.273 | 73.723 | 58.623 | 1.00 | 103.40 | 6 |
| ATOM | 20248 | OG | SER | N | 1479 | 76.032 | 75.088 | 58.327 | 1.00 | 106.29 | 8 |
| ATOM | 20249 | C | SER | N | 1479 | 77.695 | 71.809 | 57.933 | 1.00 | 40.61 | 6 |
| ATOM | 20250 | O | SER | N | 1479 | 77.139 | 70.843 | 58.454 | 1.00 | 40.38 | 8 |
| ATOM | 20251 | N | ASP | N | 1480 | 79.012 | 71.893 | 57.780 | 1.00 | 35.78 | 7 |
| ATOM | 20252 | CA | ASP | N | 1480 | 79.881 | 70.825 | 58.248 | 1.00 | 38.88 | 6 |
| ATOM | 20253 | CB | ASP | N | 1480 | 81.345 | 71.232 | 58.137 | 1.00 | 103.63 | 6 |
| ATOM | 20254 | CG | ASP | N | 1480 | 81.699 | 71.722 | 56.761 | 1.00 | 107.23 | 6 |
| ATOM | 20255 | OD1 | ASP | N | 1480 | 81.244 | 71.093 | 55.779 | 1.00 | 108.03 | 8 |
| ATOM | 20256 | OD2 | ASP | N | 1480 | 82.436 | 72.728 | 56.661 | 1.00 | 108.79 | 8 |
| ATOM | 20257 | C | ASP | N | 1480 | 79.542 | 70.545 | 59.703 | 1.00 | 39.79 | 6 |
| ATOM | 20258 | O | ASP | N | 1480 | 79.769 | 69.439 | 60.210 | 1.00 | 40.17 | 8 |
| ATOM | 20259 | N | PHE | N | 1481 | 78.993 | 71.549 | 60.380 | 1.00 | 40.34 | 7 |
| ATOM | 20260 | CA | PHE | N | 1481 | 78.626 | 71.366 | 61.763 | 1.00 | 41.24 | 6 |
| ATOM | 20261 | CB | PHE | N | 1481 | 78.029 | 72.619 | 62.371 | 1.00 | 47.30 | 6 |
| ATOM | 20262 | CG | PHE | N | 1481 | 77.659 | 72.437 | 63.796 | 1.00 | 47.35 | 6 |
| ATOM | 20263 | CD1 | PHE | N | 1481 | 78.645 | 72.292 | 64.759 | 1.00 | 47.07 | 6 |
| ATOM | 20264 | CD2 | PHE | N | 1481 | 76.335 | 72.264 | 64.161 | 1.00 | 47.62 | 6 |
| ATOM | 20265 | CE1 | PHE | N | 1481 | 78.320 | 71.968 | 66.065 | 1.00 | 47.25 | 6 |
| ATOM | 20266 | CE2 | PHE | N | 1481 | 75.995 | 71.939 | 65.462 | 1.00 | 47.72 | 6 |
| ATOM | 20267 | CZ | PHE | N | 1481 | 76.989 | 71.786 | 66.420 | 1.00 | 47.42 | 6 |
| ATOM | 20268 | C | PHE | N | 1481 | 77.594 | 70.268 | 61.802 | 1.00 | 43.00 | 6 |
| ATOM | 20269 | O | PHE | N | 1481 | 77.103 | 69.884 | 62.867 | 1.00 | 43.61 | 8 |
| ATOM | 20270 | N | VAL | N | 1482 | 77.246 | 69.777 | 60.620 | 1.00 | 18.61 | 7 |
| ATOM | 20271 | CA | VAL | N | 1482 | 76.295 | 68.684 | 60.508 | 1.00 | 20.44 | 6 |
| ATOM | 20272 | CB | VAL | N | 1482 | 74.867 | 69.188 | 60.406 | 1.00 | 13.87 | 6 |
| ATOM | 20273 | CG1 | VAL | N | 1482 | 74.013 | 68.067 | 59.914 | 1.00 | 14.05 | 6 |
| ATOM | 20274 | CG2 | VAL | N | 1482 | 74.373 | 69.672 | 61.791 | 1.00 | 13.87 | 6 |
| ATOM | 20275 | C | VAL | N | 1482 | 76.603 | 67.803 | 59.298 | 1.00 | 22.04 | 6 |
| ATOM | 20276 | O | VAL | N | 1482 | 76.646 | 68.282 | 58.163 | 1.00 | 20.60 | 8 |
| ATOM | 20277 | N | ARG | N | 1483 | 76.817 | 66.518 | 59.572 | 1.00 | 84.77 | 7 |
| ATOM | 20278 | CA | ARG | N | 1483 | 77.146 | 65.498 | 58.573 | 1.00 | 88.49 | 6 |
| ATOM | 20279 | CB | ARG | N | 1483 | 78.210 | 66.009 | 57.595 | 1.00 | 128.16 | 6 |
| ATOM | 20280 | CG | ARG | N | 1483 | 77.691 | 66.726 | 56.344 | 1.00 | 131.30 | 6 |
| ATOM | 20281 | CD | ARG | N | 1483 | 78.849 | 67.421 | 55.628 | 1.00 | 134.10 | 6 |
| ATOM | 20282 | NE | ARG | N | 1483 | 78.475 | 68.033 | 54.355 | 1.00 | 134.95 | 7 |
| ATOM | 20283 | CZ | ARG | N | 1483 | 79.325 | 68.698 | 53.577 | 1.00 | 134.62 | 6 |
| ATOM | 20284 | NH1 | ARG | N | 1483 | 80.591 | 68.834 | 53.949 | 1.00 | 133.57 | 7 |
| ATOM | 20285 | NH2 | ARG | N | 1483 | 78.917 | 69.220 | 52.426 | 1.00 | 134.01 | 7 |
| ATOM | 20286 | C | ARG | N | 1483 | 77.717 | 64.287 | 59.331 | 1.00 | 89.29 | 6 |
| ATOM | 20287 | O | ARG | N | 1483 | 78.645 | 63.624 | 58.861 | 1.00 | 89.58 | 8 |
| ATOM | 20288 | N | PHE | N | 1484 | 77.153 | 64.010 | 60.503 | 1.00 | 83.42 | 7 |
| ATOM | 20289 | CA | PHE | N | 1484 | 77.595 | 62.911 | 61.355 | 1.00 | 84.53 | 6 |
| ATOM | 20290 | CB | PHE | N | 1484 | 77.049 | 63.105 | 62.764 | 1.00 | 140.17 | 6 |
| ATOM | 20291 | CG | PHE | N | 1484 | 75.823 | 63.938 | 62.801 | 1.00 | 142.07 | 6 |
| ATOM | 20292 | CD1 | PHE | N | 1484 | 74.663 | 63.506 | 62.182 | 1.00 | 143.59 | 6 |
| ATOM | 20293 | CD2 | PHE | N | 1484 | 75.847 | 65.196 | 63.383 | 1.00 | 142.71 | 6 |
| ATOM | 20294 | CE1 | PHE | N | 1484 | 73.552 | 64.323 | 62.122 | 1.00 | 144.75 | 6 |
| ATOM | 20295 | CE2 | PHE | N | 1484 | 74.744 | 66.016 | 63.328 | 1.00 | 144.32 | 6 |
| ATOM | 20296 | CZ | PHE | N | 1484 | 73.591 | 65.580 | 62.700 | 1.00 | 144.79 | 6 |
| ATOM | 20297 | C | PHE | N | 1484 | 77.219 | 61.529 | 60.880 | 1.00 | 85.03 | 6 |
| ATOM | 20298 | O | PHE | N | 1484 | 76.295 | 61.355 | 60.082 | 1.00 | 85.54 | 8 |
| ATOM | 20299 | N | THR | N | 1485 | 77.957 | 60.549 | 61.399 | 1.00 | 134.51 | 7 |
| ATOM | 20300 | CA | THR | N | 1485 | 77.750 | 59.139 | 61.091 | 1.00 | 134.23 | 6 |
| ATOM | 20301 | CB | THR | N | 1485 | 78.650 | 58.249 | 61.982 | 1.00 | 93.85 | 6 |
| ATOM | 20302 | OG1 | THR | N | 1485 | 80.028 | 58.545 | 61.719 | 1.00 | 93.85 | 8 |
| ATOM | 20303 | CG2 | THR | N | 1485 | 78.402 | 56.786 | 61.697 | 1.00 | 94.16 | 6 |
| ATOM | 20304 | C | THR | N | 1485 | 76.285 | 58.871 | 61.391 | 1.00 | 134.08 | 6 |
| ATOM | 20305 | O | THR | N | 1485 | 75.561 | 58.296 | 60.580 | 1.00 | 134.47 | 8 |
| ATOM | 20306 | N | ALA | N | 1486 | 75.872 | 59.309 | 62.574 | 1.00 | 66.77 | 7 |
| ATOM | 20307 | CA | ALA | N | 1486 | 74.506 | 59.197 | 63.043 | 1.00 | 66.38 | 6 |
| ATOM | 20308 | CB | ALA | N | 1486 | 73.726 | 60.410 | 62.571 | 1.00 | 114.27 | 6 |
| ATOM | 20309 | C | ALA | N | 1486 | 73.745 | 57.927 | 62.686 | 1.00 | 66.41 | 6 |
| ATOM | 20310 | O | ALA | N | 1486 | 72.555 | 57.988 | 62.368 | 1.00 | 66.19 | 8 |
| ATOM | 20311 | N | VAL | N | 1487 | 74.405 | 56.774 | 62.738 | 1.00 | 59.05 | 7 |
| ATOM | 20312 | CA | VAL | N | 1487 | 73.720 | 55.516 | 62.421 | 1.00 | 59.37 | 6 |
| ATOM | 20313 | CB | VAL | N | 1487 | 73.994 | 55.049 | 60.954 | 1.00 | 98.48 | 6 |
| ATOM | 20314 | CG1 | VAL | N | 1487 | 73.064 | 53.895 | 60.578 | 1.00 | 98.08 | 6 |
| ATOM | 20315 | CG2 | VAL | N | 1487 | 73.784 | 56.195 | 59.999 | 1.00 | 98.69 | 6 |
| ATOM | 20316 | C | VAL | N | 1487 | 74.106 | 54.384 | 63.373 | 1.00 | 59.66 | 6 |
| ATOM | 20317 | O | VAL | N | 1487 | 75.019 | 53.611 | 63.113 | 1.00 | 59.03 | 8 |
| ATOM | 20318 | N | ALA | N | 1488 | 73.411 | 54.305 | 64.491 | 1.00 | 68.33 | 7 |
| ATOM | 20319 | CA | ALA | N | 1488 | 73.665 | 53.252 | 65.444 | 1.00 | 69.99 | 6 |
| ATOM | 20320 | CB | ALA | N | 1488 | 74.141 | 53.834 | 66.752 | 1.00 | 90.26 | 6 |
| ATOM | 20321 | C | ALA | N | 1488 | 72.307 | 52.589 | 65.606 | 1.00 | 71.13 | 6 |
| ATOM | 20322 | O | ALA | N | 1488 | 71.356 | 52.958 | 64.919 | 1.00 | 71.37 | 8 |
| ATOM | 20323 | N | ALA | N | 1489 | 72.206 | 51.610 | 66.496 | 1.00 | 79.81 | 7 |
| ATOM | 20324 | CA | ALA | N | 1489 | 70.940 | 50.919 | 66.721 | 1.00 | 80.53 | 6 |
| ATOM | 20325 | CB | ALA | N | 1489 | 70.651 | 49.953 | 65.553 | 1.00 | 13.87 | 6 |
| ATOM | 20326 | C | ALA | N | 1489 | 71.014 | 50.155 | 68.036 | 1.00 | 81.77 | 6 |
| ATOM | 20327 | O | ALA | N | 1489 | 70.143 | 49.342 | 68.339 | 1.00 | 82.54 | 8 |
| ATOM | 20328 | N | ALA | N | 1490 | 72.053 | 50.440 | 68.819 | 1.00 | 140.13 | 7 |
| ATOM | 20329 | CA | ALA | N | 1490 | 72.285 | 49.762 | 70.093 | 1.00 | 141.33 | 6 |
| ATOM | 20330 | CB | ALA | N | 1490 | 71.019 | 49.773 | 70.954 | 1.00 | 60.30 | 6 |
| ATOM | 20331 | C | ALA | N | 1490 | 72.691 | 48.331 | 69.762 | 1.00 | 142.29 | 6 |
| ATOM | 20332 | O | ALA | N | 1490 | 73.817 | 47.918 | 70.023 | 1.00 | 142.26 | 8 |
| ATOM | 20333 | N | ALA | N | 1491 | 71.765 | 47.587 | 69.168 | 1.00 | 183.88 | 7 |
| ATOM | 20334 | CA | ALA | N | 1491 | 72.023 | 46.212 | 68.770 | 1.00 | 185.16 | 6 |
| ATOM | 20335 | CB | ALA | N | 1491 | 70.736 | 45.564 | 68.292 | 1.00 | 69.81 | 6 |
| ATOM | 20336 | C | ALA | N | 1491 | 73.058 | 46.227 | 67.647 | 1.00 | 186.29 | 6 |
| ATOM | 20337 | O | ALA | N | 1491 | 74.142 | 45.654 | 67.775 | 1.00 | 186.59 | 8 |
| ATOM | 20338 | N | ALA | N | 1492 | 72.714 | 46.894 | 66.548 | 1.00 | 98.54 | 7 |
| ATOM | 20339 | CA | ALA | N | 1492 | 73.608 | 47.007 | 65.403 | 1.00 | 99.18 | 6 |
| ATOM | 20340 | CB | ALA | N | 1492 | 72.869 | 47.617 | 64.221 | 1.00 | 79.26 | 6 |
| ATOM | 20341 | C | ALA | N | 1492 | 74.803 | 47.872 | 65.779 | 1.00 | 100.00 | 6 |
| ATOM | 20342 | O | ALA | N | 1492 | 75.558 | 48.322 | 64.919 | 1.00 | 100.52 | 8 |
| ATOM | 20343 | N | ALA | N | 1493 | 74.953 | 48.109 | 67.077 | 1.00 | 152.21 | 7 |
| ATOM | 20344 | CA | ALA | N | 1493 | 76.053 | 48.903 | 67.602 | 1.00 | 153.89 | 6 |
| ATOM | 20345 | CB | ALA | N | 1493 | 75.529 | 49.935 | 68.591 | 1.00 | 143.28 | 6 |
| ATOM | 20346 | C | ALA | N | 1493 | 77.017 | 47.951 | 68.293 | 1.00 | 154.75 | 6 |
| ATOM | 20347 | O | ALA | N | 1493 | 78.227 | 48.174 | 68.319 | 1.00 | 155.15 | 8 |
| ATOM | 20348 | N | ALA | N | 1494 | 76.462 | 46.884 | 68.856 | 1.00 | 168.39 | 7 |
| ATOM | 20349 | CA | ALA | N | 1494 | 77.261 | 45.877 | 69.534 | 1.00 | 168.85 | 6 |
| ATOM | 20350 | CB | ALA | N | 1494 | 76.362 | 44.928 | 70.302 | 1.00 | 88.46 | 6 |
| ATOM | 20351 | C | ALA | N | 1494 | 78.032 | 45.112 | 68.471 | 1.00 | 169.44 | 6 |
| ATOM | 20352 | O | ALA | N | 1494 | 79.202 | 44.777 | 68.653 | 1.00 | 170.15 | 8 |
| ATOM | 20353 | N | ALA | N | 1495 | 77.360 | 44.847 | 67.355 | 1.00 | 107.85 | 7 |
| ATOM | 20354 | CA | ALA | N | 1495 | 77.957 | 44.114 | 66.245 | 1.00 | 108.59 | 6 |
| ATOM | 20355 | CB | ALA | N | 1495 | 76.928 | 43.941 | 65.126 | 1.00 | 99.66 | 6 |
| ATOM | 20356 | C | ALA | N | 1495 | 79.218 | 44.790 | 65.702 | 1.00 | 109.01 | 6 |
| ATOM | 20357 | O | ALA | N | 1495 | 80.188 | 44.122 | 65.343 | 1.00 | 109.17 | 8 |
| ATOM | 20358 | N | ALA | N | 1496 | 79.202 | 46.115 | 65.646 | 1.00 | 117.72 | 7 |
| ATOM | 20359 | CA | ALA | N | 1496 | 80.348 | 46.856 | 65.142 | 1.00 | 118.58 | 6 |
| ATOM | 20360 | CB | ALA | N | 1496 | 79.910 | 48.237 | 64.669 | 1.00 | 83.39 | 6 |
| ATOM | 20361 | C | ALA | N | 1496 | 81.429 | 46.985 | 66.208 | 1.00 | 119.53 | 6 |
| ATOM | 20362 | O | ALA | N | 1496 | 82.460 | 47.617 | 65.982 | 1.00 | 119.91 | 8 |
| ATOM | 20363 | N | ALA | N | 1497 | 81.195 | 46.383 | 67.369 | 1.00 | 181.59 | 7 |
| ATOM | 20364 | CA | ALA | N | 1497 | 82.166 | 46.441 | 68.456 | 1.00 | 182.69 | 6 |
| ATOM | 20365 | CB | ALA | N | 1497 | 81.580 | 47.196 | 69.641 | 1.00 | 142.39 | 6 |
| ATOM | 20366 | C | ALA | N | 1497 | 82.609 | 45.046 | 68.887 | 1.00 | 182.80 | 6 |
| ATOM | 20367 | O | ALA | N | 1497 | 83.823 | 44.770 | 68.794 | 1.00 | 183.54 | 8 |
| ATOM | 20368 | OXT | ALA | N | 1497 | 81.746 | 44.248 | 69.313 | 1.00 | 143.12 | 8 |
| ATOM | 20369 | CB | ALA | H | 1 | 49.035 | 69.727 | 66.035 | 1.00 | 112.96 | 6 |
| ATOM | 20370 | C | ALA | H | 1 | 49.981 | 69.624 | 63.746 | 1.00 | 72.10 | 6 |
| ATOM | 20371 | O | ALA | H | 1 | 50.157 | 70.628 | 63.051 | 1.00 | 71.61 | 8 |
| ATOM | 20372 | N | ALA | H | 1 | 48.217 | 68.062 | 64.422 | 1.00 | 71.93 | 7 |
| ATOM | 20373 | CA | ALA | H | 1 | 48.724 | 69.457 | 64.581 | 1.00 | 71.95 | 6 |
| ATOM | 20374 | N | ALA | H | 2 | 50.850 | 68.621 | 63.833 | 1.00 | 134.16 | 7 |
| ATOM | 20375 | CA | ALA | H | 2 | 52.107 | 68.587 | 63.096 | 1.00 | 133.75 | 6 |
| ATOM | 20376 | CB | ALA | H | 2 | 53.179 | 69.381 | 63.841 | 1.00 | 71.36 | 6 |
| ATOM | 20377 | C | ALA | H | 2 | 52.544 | 67.128 | 62.915 | 1.00 | 133.97 | 6 |
| ATOM | 20378 | O | ALA | H | 2 | 51.817 | 66.320 | 62.332 | 1.00 | 134.70 | 8 |
| ATOM | 20379 | N | GLU | H | 3 | 53.721 | 66.789 | 63.427 | 1.00 | 105.72 | 7 |
| ATOM | 20380 | CA | GLU | H | 3 | 54.237 | 65.432 | 63.302 | 1.00 | 104.48 | 6 |
| ATOM | 20381 | CB | GLU | H | 3 | 55.663 | 65.487 | 62.749 | 1.00 | 120.78 | 6 |
| ATOM | 20382 | CG | GLU | H | 3 | 55.797 | 66.283 | 61.455 | 1.00 | 123.15 | 6 |
| ATOM | 20383 | CD | GLU | H | 3 | 55.151 | 65.605 | 60.257 | 1.00 | 124.33 | 6 |
| ATOM | 20384 | OE1 | GLU | H | 3 | 53.959 | 65.229 | 60.340 | 1.00 | 125.09 | 8 |
| ATOM | 20385 | OE2 | GLU | H | 3 | 55.842 | 65.461 | 59.224 | 1.00 | 125.28 | 8 |
| ATOM | 20386 | C | GLU | H | 3 | 54.208 | 64.701 | 64.649 | 1.00 | 102.31 | 6 |
| ATOM | 20387 | O | GLU | H | 3 | 53.635 | 65.195 | 65.620 | 1.00 | 102.17 | 8 |
| ATOM | 20388 | N | PRO | H | 4 | 54.801 | 63.498 | 64.717 | 1.00 | 90.20 | 7 |
| ATOM | 20389 | CD | PRO | H | 4 | 55.167 | 62.633 | 63.579 | 1.00 | 84.46 | 6 |
| ATOM | 20390 | CA | PRO | H | 4 | 54.826 | 62.729 | 65.966 | 1.00 | 88.65 | 6 |
| ATOM | 20391 | CB | PRO | H | 4 | 55.444 | 61.404 | 65.533 | 1.00 | 83.40 | 6 |
| ATOM | 20392 | CG | PRO | H | 4 | 54.938 | 61.256 | 64.138 | 1.00 | 83.61 | 6 |
| ATOM | 20393 | C | PRO | H | 4 | 55.617 | 63.385 | 67.100 | 1.00 | 87.20 | 6 |
| ATOM | 20394 | O | PRO | H | 4 | 56.449 | 62.732 | 67.733 | 1.00 | 87.10 | 8 |
| ATOM | 20395 | N | GLY | H | 5 | 55.356 | 64.671 | 67.342 | 1.00 | 88.38 | 7 |
| ATOM | 20396 | CA | GLY | H | 5 | 56.030 | 65.409 | 68.404 | 1.00 | 86.20 | 6 |
| ATOM | 20397 | C | GLY | H | 5 | 57.320 | 66.138 | 68.045 | 1.00 | 84.54 | 6 |
| ATOM | 20398 | O | GLY | H | 5 | 58.320 | 65.977 | 68.742 | 1.00 | 84.56 | 8 |
| ATOM | 20399 | N | ILE | H | 6 | 57.297 | 66.955 | 66.988 | 1.00 | 52.67 | 7 |
| ATOM | 20400 | CA | ILE | H | 6 | 58.481 | 67.693 | 66.536 | 1.00 | 50.67 | 6 |
| ATOM | 20401 | CB | ILE | H | 6 | 58.143 | 68.779 | 65.513 | 1.00 | 61.49 | 6 |
| ATOM | 20402 | CG2 | ILE | H | 6 | 59.427 | 69.435 | 65.030 | 1.00 | 60.62 | 6 |
| ATOM | 20403 | CG1 | ILE | H | 6 | 57.418 | 68.176 | 64.317 | 1.00 | 62.92 | 6 |
| ATOM | 20404 | CD1 | ILE | H | 6 | 58.336 | 67.421 | 63.372 | 1.00 | 64.69 | 6 |
| ATOM | 20405 | C | ILE | H | 6 | 59.199 | 68.396 | 67.659 | 1.00 | 48.98 | 6 |
| ATOM | 20406 | O | ILE | H | 6 | 60.416 | 68.305 | 67.781 | 1.00 | 49.28 | 8 |
| ATOM | 20407 | N | ASP | H | 7 | 58.442 | 69.125 | 68.467 | 1.00 | 44.73 | 7 |
| ATOM | 20408 | CA | ASP | H | 7 | 59.032 | 69.855 | 69.576 | 1.00 | 43.88 | 6 |
| ATOM | 20409 | CB | ASP | H | 7 | 57.935 | 70.446 | 70.472 | 1.00 | 93.66 | 6 |
| ATOM | 20410 | CG | ASP | H | 7 | 57.368 | 71.754 | 69.923 | 1.00 | 96.14 | 6 |
| ATOM | 20411 | OD1 | ASP | H | 7 | 58.089 | 72.775 | 69.926 | 1.00 | 97.83 | 8 |
| ATOM | 20412 | OD2 | ASP | H | 7 | 56.202 | 71.766 | 69.483 | 1.00 | 96.45 | 8 |
| ATOM | 20413 | C | ASP | H | 7 | 59.927 | 68.929 | 70.371 | 1.00 | 41.56 | 6 |
| ATOM | 20414 | O | ASP | H | 7 | 61.016 | 69.308 | 70.783 | 1.00 | 40.75 | 8 |
| ATOM | 20415 | N | LYS | H | 8 | 59.466 | 67.701 | 70.558 | 1.00 | 30.16 | 7 |
| ATOM | 20416 | CA | LYS | H | 8 | 60.217 | 66.713 | 71.314 | 1.00 | 29.00 | 6 |
| ATOM | 20417 | CB | LYS | H | 8 | 59.405 | 65.418 | 71.412 | 1.00 | 163.90 | 6 |
| ATOM | 20418 | CG | LYS | H | 8 | 57.941 | 65.640 | 71.785 | 1.00 | 166.14 | 6 |
| ATOM | 20419 | CD | LYS | H | 8 | 57.143 | 64.338 | 71.831 | 1.00 | 167.38 | 6 |
| ATOM | 20420 | CE | LYS | H | 8 | 57.576 | 63.440 | 72.983 | 1.00 | 167.20 | 6 |
| ATOM | 20421 | NZ | LYS | H | 8 | 56.760 | 62.192 | 73.053 | 1.00 | 166.69 | 7 |
| ATOM | 20422 | C | LYS | H | 8 | 61.571 | 66.435 | 70.659 | 1.00 | 27.00 | 6 |
| ATOM | 20423 | O | LYS | H | 8 | 62.599 | 66.358 | 71.326 | 1.00 | 25.22 | 8 |
| ATOM | 20424 | N | LEU | H | 9 | 61.566 | 66.290 | 69.344 | 1.00 | 66.58 | 7 |
| ATOM | 20425 | CA | LEU | H | 9 | 62.789 | 66.009 | 68.616 | 1.00 | 65.94 | 6 |
| ATOM | 20426 | CB | LEU | H | 9 | 62.450 | 65.695 | 67.163 | 1.00 | 21.16 | 6 |
| ATOM | 20427 | CG | LEU | H | 9 | 61.299 | 64.705 | 66.971 | 1.00 | 19.23 | 6 |
| ATOM | 20428 | CD1 | LEU | H | 9 | 60.943 | 64.599 | 65.493 | 1.00 | 18.22 | 6 |
| ATOM | 20429 | CD2 | LEU | H | 9 | 61.687 | 63.358 | 67.546 | 1.00 | 18.68 | 6 |
| ATOM | 20430 | C | LEU | H | 9 | 63.736 | 67.200 | 68.691 | 1.00 | 66.72 | 6 |
| ATOM | 20431 | O | LEU | H | 9 | 64.894 | 67.070 | 69.089 | 1.00 | 67.80 | 8 |
| ATOM | 20432 | N | PHE | H | 10 | 63.231 | 68.366 | 68.309 | 1.00 | 58.43 | 7 |
| ATOM | 20433 | CA | PHE | H | 10 | 64.017 | 69.589 | 68.326 | 1.00 | 57.27 | 6 |
| ATOM | 20434 | CB | PHE | H | 10 | 63.131 | 70.775 | 67.984 | 1.00 | 26.49 | 6 |
| ATOM | 20435 | CG | PHE | H | 10 | 62.910 | 70.956 | 66.522 | 1.00 | 24.11 | 6 |
| ATOM | 20436 | CD1 | PHE | H | 10 | 61.743 | 71.532 | 66.057 | 1.00 | 23.18 | 6 |
| ATOM | 20437 | CD2 | PHE | H | 10 | 63.891 | 70.596 | 65.608 | 1.00 | 24.17 | 6 |
| ATOM | 20438 | CE1 | PHE | H | 10 | 61.555 | 71.750 | 64.711 | 1.00 | 23.51 | 6 |
| ATOM | 20439 | CE2 | PHE | H | 10 | 63.710 | 70.812 | 64.255 | 1.00 | 24.06 | 6 |
| ATOM | 20440 | CZ | PHE | H | 10 | 62.540 | 71.391 | 63.806 | 1.00 | 23.71 | 6 |
| ATOM | 20441 | C | PHE | H | 10 | 64.658 | 69.814 | 69.674 | 1.00 | 58.57 | 6 |
| ATOM | 20442 | O | PHE | H | 10 | 65.482 | 70.706 | 69.835 | 1.00 | 60.31 | 8 |
| ATOM | 20443 | N | GLY | H | 11 | 64.259 | 69.013 | 70.651 | 1.00 | 54.15 | 7 |
| ATOM | 20444 | CA | GLY | H | 11 | 64.834 | 69.139 | 71.973 | 1.00 | 55.56 | 6 |
| ATOM | 20445 | C | GLY | H | 11 | 66.113 | 68.342 | 71.980 | 1.00 | 56.47 | 6 |
| ATOM | 20446 | O | GLY | H | 11 | 67.191 | 68.874 | 72.245 | 1.00 | 55.94 | 8 |
| ATOM | 20447 | N | MET | H | 12 | 65.987 | 67.058 | 71.664 | 1.00 | 87.20 | 7 |
| ATOM | 20448 | CA | MET | H | 12 | 67.129 | 66.159 | 71.620 | 1.00 | 88.91 | 6 |
| ATOM | 20449 | CB | MET | H | 12 | 66.763 | 64.885 | 70.857 | 1.00 | 96.90 | 6 |
| ATOM | 20450 | CG | MET | H | 12 | 65.521 | 64.194 | 71.372 | 1.00 | 98.92 | 6 |
| ATOM | 20451 | SD | MET | H | 12 | 65.249 | 62.631 | 70.536 | 1.00 | 102.56 | 16 |
| ATOM | 20452 | CE | MET | H | 12 | 64.730 | 63.194 | 68.925 | 1.00 | 100.52 | 6 |
| ATOM | 20453 | C | MET | H | 12 | 68.332 | 66.820 | 70.953 | 1.00 | 89.24 | 6 |
| ATOM | 20454 | O | MET | H | 12 | 69.283 | 67.229 | 71.627 | 1.00 | 89.46 | 8 |
| ATOM | 20455 | N | VAL | H | 13 | 68.283 | 66.926 | 69.627 | 1.00 | 55.36 | 7 |
| ATOM | 20456 | CA | VAL | H | 13 | 69.376 | 67.528 | 68.871 | 1.00 | 55.34 | 6 |
| ATOM | 20457 | CB | VAL | H | 13 | 69.072 | 67.517 | 67.349 | 1.00 | 87.88 | 6 |
| ATOM | 20458 | CG1 | VAL | H | 13 | 69.008 | 66.091 | 66.838 | 1.00 | 87.79 | 6 |
| ATOM | 20459 | CG2 | VAL | H | 13 | 67.760 | 68.203 | 67.081 | 1.00 | 88.70 | 6 |
| ATOM | 20460 | C | VAL | H | 13 | 69.657 | 68.964 | 69.329 | 1.00 | 54.34 | 6 |
| ATOM | 20461 | O | VAL | H | 13 | 68.882 | 69.545 | 70.097 | 1.00 | 53.82 | 8 |
| ATOM | 20462 | N | ASP | H | 14 | 70.778 | 69.521 | 68.871 | 1.00 | 80.64 | 7 |
| ATOM | 20463 | CA | ASP | H | 14 | 71.163 | 70.887 | 69.220 | 1.00 | 80.29 | 6 |
| ATOM | 20464 | CB | ASP | H | 14 | 72.656 | 70.970 | 69.511 | 1.00 | 82.79 | 6 |
| ATOM | 20465 | CG | ASP | H | 14 | 73.494 | 70.843 | 68.261 | 1.00 | 84.35 | 6 |
| ATOM | 20466 | OD1 | ASP | H | 14 | 73.491 | 69.757 | 67.649 | 1.00 | 85.24 | 8 |
| ATOM | 20467 | OD2 | ASP | H | 14 | 74.141 | 71.837 | 67.879 | 1.00 | 84.99 | 8 |
| ATOM | 20468 | C | ASP | H | 14 | 70.851 | 71.784 | 68.037 | 1.00 | 78.85 | 6 |
| ATOM | 20469 | O | ASP | H | 14 | 70.039 | 72.700 | 68.140 | 1.00 | 79.58 | 8 |
| ATOM | 20470 | N | SER | H | 15 | 71.510 | 71.508 | 66.915 | 1.00 | 40.97 | 7 |
| ATOM | 20471 | CA | SER | H | 15 | 71.317 | 72.266 | 65.689 | 1.00 | 37.59 | 6 |
| ATOM | 20472 | CB | SER | H | 15 | 72.552 | 72.139 | 64.794 | 1.00 | 34.50 | 6 |
| ATOM | 20473 | OG | SER | H | 15 | 72.529 | 73.107 | 63.756 | 1.00 | 31.69 | 8 |
| ATOM | 20474 | C | SER | H | 15 | 70.089 | 71.724 | 64.961 | 1.00 | 36.60 | 6 |
| ATOM | 20475 | O | SER | H | 15 | 70.110 | 70.608 | 64.458 | 1.00 | 36.72 | 8 |
| ATOM | 20476 | N | LYS | H | 16 | 69.020 | 72.515 | 64.931 | 1.00 | 45.79 | 7 |
| ATOM | 20477 | CA | LYS | H | 16 | 67.771 | 72.145 | 64.268 | 1.00 | 45.58 | 6 |
| ATOM | 20478 | CB | LYS | H | 16 | 66.979 | 73.409 | 63.928 | 1.00 | 60.78 | 6 |
| ATOM | 20479 | CG | LYS | H | 16 | 65.704 | 73.187 | 63.138 | 1.00 | 60.97 | 6 |
| ATOM | 20480 | CD | LYS | H | 16 | 65.193 | 74.496 | 62.524 | 1.00 | 61.73 | 6 |
| ATOM | 20481 | CE | LYS | H | 16 | 63.842 | 74.310 | 61.830 | 1.00 | 62.66 | 6 |
| ATOM | 20482 | NZ | LYS | H | 16 | 63.377 | 75.511 | 61.077 | 1.00 | 62.70 | 7 |
| ATOM | 20483 | C | LYS | H | 16 | 67.993 | 71.325 | 62.995 | 1.00 | 45.74 | 6 |
| ATOM | 20484 | O | LYS | H | 16 | 67.141 | 70.530 | 62.613 | 1.00 | 45.21 | 8 |
| ATOM | 20485 | N | TYR | H | 17 | 69.136 | 71.510 | 62.343 | 1.00 | 27.19 | 7 |
| ATOM | 20486 | CA | TYR | H | 17 | 69.438 | 70.778 | 61.113 | 1.00 | 27.85 | 6 |
| ATOM | 20487 | CB | TYR | H | 17 | 70.414 | 71.575 | 60.246 | 1.00 | 54.79 | 6 |
| ATOM | 20488 | CG | TYR | H | 17 | 69.883 | 72.940 | 59.911 | 1.00 | 53.62 | 6 |
| ATOM | 20489 | CD1 | TYR | H | 17 | 69.640 | 73.858 | 60.915 | 1.00 | 52.27 | 6 |
| ATOM | 20490 | CE1 | TYR | H | 17 | 69.090 | 75.079 | 60.641 | 1.00 | 51.90 | 6 |
| ATOM | 20491 | CD2 | TYR | H | 17 | 69.563 | 73.289 | 58.607 | 1.00 | 53.37 | 6 |
| ATOM | 20492 | CE2 | TYR | H | 17 | 69.008 | 74.517 | 58.320 | 1.00 | 52.95 | 6 |
| ATOM | 20493 | CZ | TYR | H | 17 | 68.774 | 75.408 | 59.349 | 1.00 | 52.81 | 6 |
| ATOM | 20494 | OH | TYR | H | 17 | 68.218 | 76.638 | 59.111 | 1.00 | 53.12 | 8 |
| ATOM | 20495 | C | TYR | H | 17 | 70.003 | 69.407 | 61.391 | 1.00 | 28.58 | 6 |
| ATOM | 20496 | O | TYR | H | 17 | 70.093 | 68.576 | 60.497 | 1.00 | 28.23 | 8 |
| ATOM | 20497 | N | ARG | H | 18 | 70.397 | 69.191 | 62.638 | 1.00 | 55.84 | 7 |
| ATOM | 20498 | CA | ARG | H | 18 | 70.935 | 67.910 | 63.060 | 1.00 | 56.85 | 6 |
| ATOM | 20499 | CB | ARG | H | 18 | 71.381 | 67.973 | 64.532 | 1.00 | 86.52 | 6 |
| ATOM | 20500 | CG | ARG | H | 18 | 71.976 | 66.678 | 65.097 | 1.00 | 87.17 | 6 |
| ATOM | 20501 | CD | ARG | H | 18 | 73.125 | 66.954 | 66.074 | 1.00 | 89.03 | 6 |
| ATOM | 20502 | NE | ARG | H | 18 | 74.182 | 67.758 | 65.451 | 1.00 | 91.03 | 7 |
| ATOM | 20503 | CZ | ARG | H | 18 | 75.372 | 68.007 | 65.994 | 1.00 | 90.39 | 6 |
| ATOM | 20504 | NH1 | ARG | H | 18 | 75.678 | 67.516 | 67.184 | 1.00 | 90.95 | 7 |
| ATOM | 20505 | NH2 | ARG | H | 18 | 76.260 | 68.749 | 65.345 | 1.00 | 88.61 | 7 |
| ATOM | 20506 | C | ARG | H | 18 | 69.790 | 66.924 | 62.884 | 1.00 | 56.72 | 6 |
| ATOM | 20507 | O | ARG | H | 18 | 69.828 | 66.074 | 61.993 | 1.00 | 57.05 | 8 |
| ATOM | 20508 | N | LEU | H | 19 | 68.765 | 67.062 | 63.722 | 1.00 | 68.32 | 7 |
| ATOM | 20509 | CA | LEU | H | 19 | 67.592 | 66.197 | 63.654 | 1.00 | 68.14 | 6 |
| ATOM | 20510 | CB | LEU | H | 19 | 66.440 | 66.832 | 64.429 | 1.00 | 47.50 | 6 |
| ATOM | 20511 | CG | LEU | H | 19 | 65.045 | 66.297 | 64.121 | 1.00 | 47.31 | 6 |
| ATOM | 20512 | CD1 | LEU | H | 19 | 64.966 | 64.837 | 64.491 | 1.00 | 47.18 | 6 |
| ATOM | 20513 | CD2 | LEU | H | 19 | 64.022 | 67.104 | 64.884 | 1.00 | 48.01 | 6 |
| ATOM | 20514 | C | LEU | H | 19 | 67.160 | 65.945 | 62.210 | 1.00 | 67.80 | 6 |
| ATOM | 20515 | O | LEU | H | 19 | 66.854 | 64.814 | 61.840 | 1.00 | 68.35 | 8 |
| ATOM | 20516 | N | THR | H | 20 | 67.132 | 67.002 | 61.402 | 1.00 | 51.77 | 7 |
| ATOM | 20517 | CA | THR | H | 20 | 66.751 | 66.877 | 60.000 | 1.00 | 51.37 | 6 |
| ATOM | 20518 | CB | THR | H | 20 | 66.867 | 68.228 | 59.250 | 1.00 | 52.73 | 6 |
| ATOM | 20519 | OG1 | THR | H | 20 | 65.932 | 69.165 | 59.796 | 1.00 | 53.84 | 8 |
| ATOM | 20520 | CG2 | THR | H | 20 | 66.577 | 68.047 | 57.769 | 1.00 | 52.85 | 6 |
| ATOM | 20521 | C | THR | H | 20 | 67.672 | 65.876 | 59.318 | 1.00 | 49.82 | 6 |
| ATOM | 20522 | O | THR | H | 20 | 67.218 | 64.974 | 58.623 | 1.00 | 49.18 | 8 |
| ATOM | 20523 | N | VAL | H | 21 | 68.970 | 66.044 | 59.536 | 1.00 | 56.67 | 7 |
| ATOM | 20524 | CA | VAL | H | 21 | 69.989 | 65.182 | 58.950 | 1.00 | 56.33 | 6 |
| ATOM | 20525 | CB | VAL | H | 21 | 71.363 | 65.733 | 59.252 | 1.00 | 47.80 | 6 |
| ATOM | 20526 | CG1 | VAL | H | 21 | 72.431 | 64.728 | 58.857 | 1.00 | 46.78 | 6 |
| ATOM | 20527 | CG2 | VAL | H | 21 | 71.526 | 67.049 | 58.514 | 1.00 | 49.31 | 6 |
| ATOM | 20528 | C | VAL | H | 21 | 69.949 | 63.735 | 59.401 | 1.00 | 55.74 | 6 |
| ATOM | 20529 | O | VAL | H | 21 | 69.989 | 62.826 | 58.576 | 1.00 | 56.18 | 8 |
| ATOM | 20530 | N | VAL | H | 22 | 69.897 | 63.528 | 60.712 | 1.00 | 35.41 | 7 |
| ATOM | 20531 | CA | VAL | H | 22 | 69.842 | 62.191 | 61.287 | 1.00 | 33.46 | 6 |
| ATOM | 20532 | CB | VAL | H | 22 | 69.914 | 62.261 | 62.811 | 1.00 | 29.04 | 6 |
| ATOM | 20533 | CG1 | VAL | H | 22 | 69.348 | 61.000 | 63.412 | 1.00 | 29.07 | 6 |
| ATOM | 20534 | CG2 | VAL | H | 22 | 71.345 | 62.448 | 63.248 | 1.00 | 27.73 | 6 |
| ATOM | 20535 | C | VAL | H | 22 | 68.570 | 61.445 | 60.889 | 1.00 | 33.06 | 6 |
| ATOM | 20536 | O | VAL | H | 22 | 68.629 | 60.292 | 60.491 | 1.00 | 32.88 | 8 |
| ATOM | 20537 | N | VAL | H | 23 | 67.422 | 62.095 | 61.021 | 1.00 | 26.43 | 7 |
| ATOM | 20538 | CA | VAL | H | 23 | 66.159 | 61.475 | 60.649 | 1.00 | 26.88 | 6 |
| ATOM | 20539 | CB | VAL | H | 23 | 64.966 | 62.446 | 60.831 | 1.00 | 31.77 | 6 |
| ATOM | 20540 | CG1 | VAL | H | 23 | 63.881 | 62.155 | 59.807 | 1.00 | 30.73 | 6 |
| ATOM | 20541 | CG2 | VAL | H | 23 | 64.394 | 62.318 | 62.227 | 1.00 | 31.75 | 6 |
| ATOM | 20542 | C | VAL | H | 23 | 66.233 | 61.103 | 59.186 | 1.00 | 28.31 | 6 |
| ATOM | 20543 | O | VAL | H | 23 | 65.663 | 60.100 | 58.762 | 1.00 | 29.36 | 8 |
| ATOM | 20544 | N | ALA | H | 24 | 66.941 | 61.922 | 58.418 | 1.00 | 25.22 | 7 |
| ATOM | 20545 | CA | ALA | H | 24 | 67.086 | 61.712 | 56.978 | 1.00 | 27.09 | 6 |
| ATOM | 20546 | CB | ALA | H | 24 | 67.767 | 62.901 | 56.350 | 1.00 | 29.93 | 6 |
| ATOM | 20547 | C | ALA | H | 24 | 67.854 | 60.463 | 56.623 | 1.00 | 28.67 | 6 |
| ATOM | 20548 | O | ALA | H | 24 | 67.341 | 59.595 | 55.919 | 1.00 | 28.38 | 8 |
| ATOM | 20549 | N | LYS | H | 25 | 69.097 | 60.398 | 57.097 | 1.00 | 59.88 | 7 |
| ATOM | 20550 | CA | LYS | H | 25 | 69.971 | 59.264 | 56.833 | 1.00 | 62.39 | 6 |
| ATOM | 20551 | CB | LYS | H | 25 | 71.355 | 59.504 | 57.434 | 1.00 | 104.23 | 6 |
| ATOM | 20552 | CG | LYS | H | 25 | 72.443 | 58.609 | 56.854 | 1.00 | 107.91 | 6 |
| ATOM | 20553 | CD | LYS | H | 25 | 72.234 | 57.130 | 57.207 | 1.00 | 111.99 | 6 |
| ATOM | 20554 | CE | LYS | H | 25 | 73.392 | 56.246 | 56.721 | 1.00 | 114.74 | 6 |
| ATOM | 20555 | NZ | LYS | H | 25 | 73.418 | 54.895 | 57.369 | 1.00 | 116.09 | 7 |
| ATOM | 20556 | C | LYS | H | 25 | 69.335 | 58.050 | 57.462 | 1.00 | 63.18 | 6 |
| ATOM | 20557 | O | LYS | H | 25 | 69.321 | 56.972 | 56.883 | 1.00 | 62.18 | 8 |
| ATOM | 20558 | N | ARG | H | 26 | 68.799 | 58.240 | 58.659 | 1.00 | 53.40 | 7 |
| ATOM | 20559 | CA | ARG | H | 26 | 68.143 | 57.157 | 59.377 | 1.00 | 54.97 | 6 |
| ATOM | 20560 | CB | ARG | H | 26 | 67.382 | 57.713 | 60.589 | 1.00 | 50.83 | 6 |
| ATOM | 20561 | CG | ARG | H | 26 | 66.674 | 56.645 | 61.389 | 1.00 | 48.91 | 6 |
| ATOM | 20562 | CD | ARG | H | 26 | 67.586 | 55.454 | 61.584 | 1.00 | 46.66 | 6 |
| ATOM | 20563 | NE | ARG | H | 26 | 66.826 | 54.237 | 61.809 | 1.00 | 45.56 | 7 |
| ATOM | 20564 | CZ | ARG | H | 26 | 67.335 | 53.023 | 61.689 | 1.00 | 46.00 | 6 |
| ATOM | 20565 | NH1 | ARG | H | 26 | 68.605 | 52.881 | 61.337 | 1.00 | 46.68 | 7 |
| ATOM | 20566 | NH2 | ARG | H | 26 | 66.583 | 51.962 | 61.940 | 1.00 | 45.14 | 7 |
| ATOM | 20567 | C | ARG | H | 26 | 67.186 | 56.436 | 58.438 | 1.00 | 56.51 | 6 |
| ATOM | 20568 | O | ARG | H | 26 | 67.410 | 55.293 | 58.053 | 1.00 | 56.26 | 8 |
| ATOM | 20569 | N | ALA | H | 27 | 66.112 | 57.120 | 58.075 | 1.00 | 61.12 | 7 |
| ATOM | 20570 | CA | ALA | H | 27 | 65.144 | 56.557 | 57.162 | 1.00 | 63.44 | 6 |
| ATOM | 20571 | CB | ALA | H | 27 | 64.129 | 57.633 | 56.764 | 1.00 | 48.64 | 6 |
| ATOM | 20572 | C | ALA | H | 27 | 65.940 | 56.081 | 55.948 | 1.00 | 65.57 | 6 |
| ATOM | 20573 | O | ALA | H | 27 | 65.574 | 55.099 | 55.300 | 1.00 | 65.48 | 8 |
| ATOM | 20574 | N | GLN | H | 28 | 67.043 | 56.782 | 55.672 | 1.00 | 65.54 | 7 |
| ATOM | 20575 | CA | GLN | H | 28 | 67.927 | 56.474 | 54.545 | 1.00 | 68.75 | 6 |
| ATOM | 20576 | CB | GLN | H | 28 | 68.654 | 57.731 | 54.071 | 1.00 | 110.27 | 6 |
| ATOM | 20577 | CG | GLN | H | 28 | 69.526 | 57.494 | 52.855 | 1.00 | 112.49 | 6 |
| ATOM | 20578 | CD | GLN | H | 28 | 70.453 | 58.647 | 52.568 | 1.00 | 114.71 | 6 |
| ATOM | 20579 | OE1 | GLN | H | 28 | 71.287 | 59.005 | 53.396 | 1.00 | 116.10 | 8 |
| ATOM | 20580 | NE2 | GLN | H | 28 | 70.316 | 59.237 | 51.387 | 1.00 | 116.29 | 7 |
| ATOM | 20581 | C | GLN | H | 28 | 68.964 | 55.424 | 54.921 | 1.00 | 70.11 | 6 |
| ATOM | 20582 | O | GLN | H | 28 | 70.167 | 55.652 | 54.798 | 1.00 | 70.08 | 8 |
| ATOM | 20583 | N | GLN | H | 29 | 68.479 | 54.279 | 55.387 | 1.00 | 103.66 | 7 |
| ATOM | 20584 | CA | GLN | H | 29 | 69.312 | 53.152 | 55.802 | 1.00 | 105.28 | 6 |
| ATOM | 20585 | CB | GLN | H | 29 | 70.363 | 53.610 | 56.825 | 1.00 | 193.59 | 6 |
| ATOM | 20586 | CG | GLN | H | 29 | 71.553 | 52.662 | 57.012 | 1.00 | 197.64 | 6 |
| ATOM | 20587 | CD | GLN | H | 29 | 72.482 | 52.606 | 55.800 | 1.00 | 199.79 | 6 |
| ATOM | 20588 | OE1 | GLN | H | 29 | 72.958 | 53.633 | 55.315 | 1.00 | 201.30 | 8 |
| ATOM | 20589 | NE2 | GLN | H | 29 | 72.752 | 51.399 | 55.319 | 1.00 | 199.49 | 7 |
| ATOM | 20590 | C | GLN | H | 29 | 68.315 | 52.192 | 56.447 | 1.00 | 104.60 | 6 |
| ATOM | 20591 | O | GLN | H | 29 | 68.576 | 51.001 | 56.627 | 1.00 | 103.61 | 8 |
| ATOM | 20592 | N | LEU | H | 30 | 67.162 | 52.750 | 56.793 | 1.00 | 78.51 | 7 |
| ATOM | 20593 | CA | LEU | H | 30 | 66.072 | 52.005 | 57.395 | 1.00 | 77.56 | 6 |
| ATOM | 20594 | CB | LEU | H | 30 | 65.098 | 52.984 | 58.065 | 1.00 | 76.20 | 6 |
| ATOM | 20595 | CG | LEU | H | 30 | 63.918 | 52.502 | 58.918 | 1.00 | 76.30 | 6 |
| ATOM | 20596 | CD1 | LEU | H | 30 | 64.383 | 52.072 | 60.301 | 1.00 | 76.47 | 6 |
| ATOM | 20597 | CD2 | LEU | H | 30 | 62.925 | 53.645 | 59.055 | 1.00 | 76.36 | 6 |
| ATOM | 20598 | C | LEU | H | 30 | 65.433 | 51.360 | 56.180 | 1.00 | 76.72 | 6 |
| ATOM | 20599 | O | LEU | H | 30 | 66.124 | 50.776 | 55.351 | 1.00 | 77.37 | 8 |
| ATOM | 20600 | N | LEU | H | 31 | 64.119 | 51.484 | 56.069 | 1.00 | 41.99 | 7 |
| ATOM | 20601 | CA | LEU | H | 31 | 63.377 | 50.952 | 54.933 | 1.00 | 40.88 | 6 |
| ATOM | 20602 | CB | LEU | H | 31 | 61.876 | 51.298 | 55.100 | 1.00 | 41.76 | 6 |
| ATOM | 20603 | CG | LEU | H | 31 | 60.787 | 50.270 | 55.501 | 1.00 | 40.39 | 6 |
| ATOM | 20604 | CD1 | LEU | H | 31 | 61.199 | 49.438 | 56.687 | 1.00 | 39.57 | 6 |
| ATOM | 20605 | CD2 | LEU | H | 31 | 59.499 | 51.010 | 55.827 | 1.00 | 40.61 | 6 |
| ATOM | 20606 | C | LEU | H | 31 | 63.935 | 51.527 | 53.602 | 1.00 | 40.65 | 6 |
| ATOM | 20607 | O | LEU | H | 31 | 63.234 | 51.581 | 52.592 | 1.00 | 39.31 | 8 |
| ATOM | 20608 | N | ARG | H | 32 | 65.193 | 51.966 | 53.612 | 1.00 | 78.66 | 7 |
| ATOM | 20609 | CA | ARG | H | 32 | 65.837 | 52.497 | 52.410 | 1.00 | 80.40 | 6 |
| ATOM | 20610 | CB | ARG | H | 32 | 66.870 | 53.572 | 52.786 | 1.00 | 95.88 | 6 |
| ATOM | 20611 | CG | ARG | H | 32 | 68.337 | 53.135 | 52.725 | 1.00 | 95.99 | 6 |
| ATOM | 20612 | CD | ARG | H | 32 | 69.102 | 53.870 | 51.623 | 1.00 | 95.72 | 6 |
| ATOM | 20613 | NE | ARG | H | 32 | 70.518 | 53.505 | 51.598 | 1.00 | 95.98 | 7 |
| ATOM | 20614 | CZ | ARG | H | 32 | 71.375 | 53.863 | 50.644 | 1.00 | 95.67 | 6 |
| ATOM | 20615 | NH1 | ARG | H | 32 | 70.970 | 54.603 | 49.620 | 1.00 | 95.63 | 7 |
| ATOM | 20616 | NH2 | ARG | H | 32 | 72.640 | 53.475 | 50.713 | 1.00 | 95.56 | 7 |
| ATOM | 20617 | C | ARG | H | 32 | 66.524 | 51.315 | 51.720 | 1.00 | 81.03 | 6 |
| ATOM | 20618 | O | ARG | H | 32 | 66.714 | 51.287 | 50.497 | 1.00 | 81.36 | 8 |
| ATOM | 20619 | N | HIS | H | 33 | 66.903 | 50.344 | 52.541 | 1.00 | 109.02 | 7 |
| ATOM | 20620 | CA | HIS | H | 33 | 67.542 | 49.116 | 52.087 | 1.00 | 108.53 | 6 |
| ATOM | 20621 | CB | HIS | H | 33 | 69.003 | 49.055 | 52.552 | 1.00 | 122.48 | 6 |
| ATOM | 20622 | CG | HIS | H | 33 | 69.993 | 49.546 | 51.541 | 1.00 | 124.33 | 6 |
| ATOM | 20623 | CD2 | HIS | H | 33 | 70.889 | 50.560 | 51.590 | 1.00 | 125.29 | 6 |
| ATOM | 20624 | ND1 | HIS | H | 33 | 70.174 | 48.935 | 50.320 | 1.00 | 125.38 | 7 |
| ATOM | 20625 | CE1 | HIS | H | 33 | 71.141 | 49.550 | 49.660 | 1.00 | 125.83 | 6 |
| ATOM | 20626 | NE2 | HIS | H | 33 | 71.592 | 50.540 | 50.409 | 1.00 | 125.62 | 7 |
| ATOM | 20627 | C | HIS | H | 33 | 66.761 | 47.995 | 52.762 | 1.00 | 107.63 | 6 |
| ATOM | 20628 | O | HIS | H | 33 | 65.845 | 47.405 | 52.188 | 1.00 | 108.16 | 8 |
| ATOM | 20629 | N | ALA | H | 34 | 67.128 | 47.746 | 54.011 | 1.00 | 31.83 | 7 |
| ATOM | 20630 | CA | ALA | H | 34 | 66.522 | 46.722 | 54.832 | 1.00 | 30.66 | 6 |
| ATOM | 20631 | CB | ALA | H | 34 | 67.342 | 46.556 | 56.117 | 1.00 | 42.18 | 6 |
| ATOM | 20632 | C | ALA | H | 34 | 65.070 | 47.028 | 55.168 | 1.00 | 30.40 | 6 |
| ATOM | 20633 | O | ALA | H | 34 | 64.273 | 47.361 | 54.294 | 1.00 | 29.31 | 8 |
| ATOM | 20634 | N | ALA | H | 35 | 64.747 | 46.899 | 56.449 | 1.00 | 82.73 | 7 |
| ATOM | 20635 | CA | ALA | H | 35 | 63.408 | 47.135 | 56.976 | 1.00 | 84.31 | 6 |
| ATOM | 20636 | CB | ALA | H | 35 | 62.374 | 46.385 | 56.155 | 1.00 | 112.48 | 6 |
| ATOM | 20637 | C | ALA | H | 35 | 63.444 | 46.597 | 58.405 | 1.00 | 84.82 | 6 |
| ATOM | 20638 | O | ALA | H | 35 | 62.571 | 46.872 | 59.231 | 1.00 | 85.14 | 8 |
| ATOM | 20639 | N | ALA | H | 36 | 64.491 | 45.830 | 58.678 | 1.00 | 78.81 | 7 |
| ATOM | 20640 | CA | ALA | H | 36 | 64.697 | 45.233 | 59.977 | 1.00 | 79.13 | 6 |
| ATOM | 20641 | CB | ALA | H | 36 | 65.125 | 43.816 | 59.797 | 1.00 | 13.87 | 6 |
| ATOM | 20642 | C | ALA | H | 36 | 65.714 | 45.999 | 60.808 | 1.00 | 80.29 | 6 |
| ATOM | 20643 | O | ALA | H | 36 | 66.760 | 45.469 | 61.200 | 1.00 | 80.93 | 8 |
| ATOM | 20644 | N | ASN | H | 37 | 65.407 | 47.265 | 61.055 | 1.00 | 95.62 | 7 |
| ATOM | 20645 | CA | ASN | H | 37 | 66.278 | 48.130 | 61.853 | 1.00 | 96.26 | 6 |
| ATOM | 20646 | CB | ASN | H | 37 | 67.041 | 49.105 | 60.960 | 1.00 | 60.61 | 6 |
| ATOM | 20647 | CG | ASN | H | 37 | 67.714 | 48.411 | 59.801 | 1.00 | 60.22 | 6 |
| ATOM | 20648 | OD1 | ASN | H | 37 | 68.195 | 47.283 | 59.949 | 1.00 | 60.57 | 8 |
| ATOM | 20649 | ND2 | ASN | H | 37 | 67.784 | 49.076 | 58.657 | 1.00 | 58.82 | 7 |
| ATOM | 20650 | C | ASN | H | 37 | 65.288 | 48.893 | 62.706 | 1.00 | 97.83 | 6 |
| ATOM | 20651 | O | ASN | H | 37 | 65.603 | 49.921 | 63.309 | 1.00 | 97.88 | 8 |
| ATOM | 20652 | N | THR | H | 38 | 64.069 | 48.372 | 62.734 | 1.00 | 94.83 | 7 |
| ATOM | 20653 | CA | THR | H | 38 | 62.985 | 48.962 | 63.495 | 1.00 | 96.49 | 6 |
| ATOM | 20654 | CB | THR | H | 38 | 61.680 | 48.219 | 63.200 | 1.00 | 123.38 | 6 |
| ATOM | 20655 | OG1 | THR | H | 38 | 61.414 | 48.265 | 61.790 | 1.00 | 123.47 | 8 |
| ATOM | 20656 | CG2 | THR | H | 38 | 60.526 | 48.845 | 63.963 | 1.00 | 123.16 | 6 |
| ATOM | 20657 | C | THR | H | 38 | 63.284 | 48.884 | 64.981 | 1.00 | 97.60 | 6 |
| ATOM | 20658 | O | THR | H | 38 | 64.127 | 48.109 | 65.422 | 1.00 | 98.49 | 8 |
| ATOM | 20659 | N | VAL | H | 39 | 62.582 | 49.699 | 65.751 | 1.00 | 75.84 | 7 |
| ATOM | 20660 | CA | VAL | H | 39 | 62.751 | 49.738 | 67.195 | 1.00 | 76.63 | 6 |
| ATOM | 20661 | CB | VAL | H | 39 | 63.574 | 50.982 | 67.627 | 1.00 | 75.51 | 6 |
| ATOM | 20662 | CG1 | VAL | H | 39 | 63.757 | 51.001 | 69.129 | 1.00 | 75.66 | 6 |
| ATOM | 20663 | CG2 | VAL | H | 39 | 64.921 | 50.965 | 66.920 | 1.00 | 74.82 | 6 |
| ATOM | 20664 | C | VAL | H | 39 | 61.327 | 49.873 | 67.730 | 1.00 | 77.84 | 6 |
| ATOM | 20665 | O | VAL | H | 39 | 61.088 | 49.895 | 68.937 | 1.00 | 77.57 | 8 |
| ATOM | 20666 | N | LEU | H | 40 | 60.379 | 49.945 | 66.804 | 1.00 | 122.58 | 7 |
| ATOM | 20667 | CA | LEU | H | 40 | 58.983 | 50.129 | 67.140 | 1.00 | 124.80 | 6 |
| ATOM | 20668 | CB | LEU | H | 40 | 58.451 | 51.322 | 66.357 | 1.00 | 89.47 | 6 |
| ATOM | 20669 | CG | LEU | H | 40 | 59.504 | 52.361 | 65.979 | 1.00 | 89.93 | 6 |
| ATOM | 20670 | CD1 | LEU | H | 40 | 58.894 | 53.377 | 65.035 | 1.00 | 90.11 | 6 |
| ATOM | 20671 | CD2 | LEU | H | 40 | 60.058 | 53.028 | 67.230 | 1.00 | 90.05 | 6 |
| ATOM | 20672 | C | LEU | H | 40 | 58.146 | 48.897 | 66.824 | 1.00 | 126.55 | 6 |
| ATOM | 20673 | O | LEU | H | 40 | 56.909 | 48.964 | 66.797 | 1.00 | 126.66 | 8 |
| ATOM | 20674 | N | ALA | H | 41 | 58.802 | 47.767 | 66.599 | 1.00 | 208.87 | 7 |
| ATOM | 20675 | CA | ALA | H | 41 | 58.080 | 46.544 | 66.270 | 1.00 | 208.87 | 6 |
| ATOM | 20676 | CB | ALA | H | 41 | 58.997 | 45.650 | 65.409 | 1.00 | 161.16 | 6 |
| ATOM | 20677 | C | ALA | H | 41 | 57.462 | 45.725 | 67.399 | 1.00 | 208.87 | 6 |
| ATOM | 20678 | O | ALA | H | 41 | 56.910 | 44.660 | 67.158 | 1.00 | 208.87 | 8 |
| ATOM | 20679 | N | PRO | H | 42 | 57.541 | 46.200 | 68.650 | 1.00 | 208.87 | 7 |
| ATOM | 20680 | CD | PRO | H | 42 | 58.372 | 47.229 | 69.296 | 1.00 | 169.16 | 6 |
| ATOM | 20681 | CA | PRO | H | 42 | 56.923 | 45.345 | 69.671 | 1.00 | 208.87 | 6 |
| ATOM | 20682 | CB | PRO | H | 42 | 57.598 | 45.806 | 70.979 | 1.00 | 169.61 | 6 |
| ATOM | 20683 | CG | PRO | H | 42 | 57.858 | 47.257 | 70.709 | 1.00 | 169.89 | 6 |
| ATOM | 20684 | C | PRO | H | 42 | 55.402 | 45.465 | 69.767 | 1.00 | 208.87 | 6 |
| ATOM | 20685 | O | PRO | H | 42 | 54.684 | 44.471 | 69.976 | 1.00 | 208.87 | 8 |
| ATOM | 20686 | N | ALA | H | 43 | 54.912 | 46.683 | 69.572 | 1.00 | 166.32 | 7 |
| ATOM | 20687 | CA | ALA | H | 43 | 53.484 | 46.970 | 69.646 | 1.00 | 165.47 | 6 |
| ATOM | 20688 | CB | ALA | H | 43 | 52.945 | 46.587 | 71.015 | 1.00 | 82.37 | 6 |
| ATOM | 20689 | C | ALA | H | 43 | 53.325 | 48.471 | 69.414 | 1.00 | 164.85 | 6 |
| ATOM | 20690 | O | ALA | H | 43 | 52.211 | 49.003 | 69.426 | 1.00 | 164.86 | 8 |
| ATOM | 20691 | N | GLU | H | 44 | 54.462 | 49.141 | 69.220 | 1.00 | 115.40 | 7 |
| ATOM | 20692 | CA | GLU | H | 44 | 54.509 | 50.579 | 68.984 | 1.00 | 114.09 | 6 |
| ATOM | 20693 | CB | GLU | H | 44 | 55.446 | 51.252 | 69.998 | 1.00 | 68.90 | 6 |
| ATOM | 20694 | CG | GLU | H | 44 | 56.815 | 50.602 | 70.134 | 1.00 | 68.56 | 6 |
| ATOM | 20695 | CD | GLU | H | 44 | 57.746 | 51.359 | 71.073 | 1.00 | 68.07 | 6 |
| ATOM | 20696 | OE1 | GLU | H | 44 | 57.288 | 51.768 | 72.154 | 1.00 | 68.52 | 8 |
| ATOM | 20697 | OE2 | GLU | H | 44 | 58.933 | 51.543 | 70.728 | 1.00 | 67.29 | 8 |
| ATOM | 20698 | C | GLU | H | 44 | 55.003 | 50.856 | 67.571 | 1.00 | 113.37 | 6 |
| ATOM | 20699 | O | GLU | H | 44 | 55.866 | 51.703 | 67.361 | 1.00 | 113.69 | 8 |
| ATOM | 20700 | N | ALA | H | 45 | 54.446 | 50.144 | 66.600 | 1.00 | 102.32 | 7 |
| ATOM | 20701 | CA | ALA | H | 45 | 54.854 | 50.318 | 65.217 | 1.00 | 101.09 | 6 |
| ATOM | 20702 | CB | ALA | H | 45 | 55.026 | 48.959 | 64.554 | 1.00 | 111.56 | 6 |
| ATOM | 20703 | C | ALA | H | 45 | 53.864 | 51.168 | 64.420 | 1.00 | 100.08 | 6 |
| ATOM | 20704 | O | ALA | H | 45 | 52.647 | 51.053 | 64.582 | 1.00 | 99.68 | 8 |
| ATOM | 20705 | N | PRO | H | 46 | 54.385 | 52.054 | 63.551 | 1.00 | 108.75 | 7 |
| ATOM | 20706 | CD | PRO | H | 46 | 55.813 | 52.404 | 63.443 | 1.00 | 70.08 | 6 |
| ATOM | 20707 | CA | PRO | H | 46 | 53.574 | 52.938 | 62.707 | 1.00 | 107.38 | 6 |
| ATOM | 20708 | CB | PRO | H | 46 | 54.627 | 53.732 | 61.934 | 1.00 | 68.99 | 6 |
| ATOM | 20709 | CG | PRO | H | 46 | 55.765 | 53.802 | 62.893 | 1.00 | 69.22 | 6 |
| ATOM | 20710 | C | PRO | H | 46 | 52.655 | 52.147 | 61.771 | 1.00 | 106.25 | 6 |
| ATOM | 20711 | O | PRO | H | 46 | 53.111 | 51.276 | 61.030 | 1.00 | 106.74 | 8 |
| ATOM | 20712 | N | LYS | H | 47 | 51.363 | 52.449 | 61.805 | 1.00 | 77.55 | 7 |
| ATOM | 20713 | CA | LYS | H | 47 | 50.398 | 51.763 | 60.955 | 1.00 | 76.32 | 6 |
| ATOM | 20714 | CB | LYS | H | 47 | 49.602 | 50.768 | 61.788 | 1.00 | 60.45 | 6 |
| ATOM | 20715 | CG | LYS | H | 47 | 50.488 | 49.829 | 62.600 | 1.00 | 59.93 | 6 |
| ATOM | 20716 | CD | LYS | H | 47 | 49.690 | 48.672 | 63.200 | 1.00 | 60.60 | 6 |
| ATOM | 20717 | CE | LYS | H | 47 | 49.929 | 47.357 | 62.445 | 1.00 | 61.71 | 6 |
| ATOM | 20718 | NZ | LYS | H | 47 | 48.915 | 46.301 | 62.780 | 1.00 | 63.05 | 7 |
| ATOM | 20719 | C | LYS | H | 47 | 49.471 | 52.779 | 60.298 | 1.00 | 76.31 | 6 |
| ATOM | 20720 | O | LYS | H | 47 | 49.301 | 53.886 | 60.798 | 1.00 | 76.41 | 8 |
| ATOM | 20721 | N | MET | H | 48 | 48.874 | 52.397 | 59.175 | 1.00 | 136.18 | 7 |
| ATOM | 20722 | CA | MET | H | 48 | 47.990 | 53.283 | 58.426 | 1.00 | 136.16 | 6 |
| ATOM | 20723 | CB | MET | H | 48 | 48.345 | 53.195 | 56.946 | 1.00 | 59.72 | 6 |
| ATOM | 20724 | CG | MET | H | 48 | 47.728 | 54.261 | 56.105 | 1.00 | 59.53 | 6 |
| ATOM | 20725 | SD | MET | H | 48 | 48.121 | 54.004 | 54.390 | 1.00 | 58.70 | 16 |
| ATOM | 20726 | CE | MET | H | 48 | 49.888 | 54.095 | 54.463 | 1.00 | 59.27 | 6 |
| ATOM | 20727 | C | MET | H | 48 | 46.503 | 52.976 | 58.618 | 1.00 | 136.90 | 6 |
| ATOM | 20728 | O | MET | H | 48 | 46.059 | 52.706 | 59.732 | 1.00 | 138.26 | 8 |
| ATOM | 20729 | N | ARG | H | 49 | 45.741 | 53.023 | 57.526 | 1.00 | 98.70 | 7 |
| ATOM | 20730 | CA | ARG | H | 49 | 44.304 | 52.740 | 57.549 | 1.00 | 99.46 | 6 |
| ATOM | 20731 | CB | ARG | H | 49 | 43.571 | 53.715 | 58.474 | 1.00 | 165.52 | 6 |
| ATOM | 20732 | CG | ARG | H | 49 | 43.250 | 53.181 | 59.864 | 1.00 | 167.81 | 6 |
| ATOM | 20733 | CD | ARG | H | 49 | 42.372 | 54.177 | 60.609 | 1.00 | 169.11 | 6 |
| ATOM | 20734 | NE | ARG | H | 49 | 42.023 | 53.740 | 61.956 | 1.00 | 170.49 | 7 |
| ATOM | 20735 | CZ | ARG | H | 49 | 41.245 | 54.432 | 62.784 | 1.00 | 171.18 | 6 |
| ATOM | 20736 | NH1 | ARG | H | 49 | 40.731 | 55.596 | 62.400 | 1.00 | 170.50 | 7 |
| ATOM | 20737 | NH2 | ARG | H | 49 | 40.979 | 53.964 | 63.996 | 1.00 | 171.83 | 7 |
| ATOM | 20738 | C | ARG | H | 49 | 43.696 | 52.840 | 56.152 | 1.00 | 99.81 | 6 |
| ATOM | 20739 | O | ARG | H | 49 | 43.645 | 53.924 | 55.569 | 1.00 | 100.57 | 8 |
| ATOM | 20740 | N | THR | H | 50 | 43.226 | 51.714 | 55.621 | 1.00 | 79.76 | 7 |
| ATOM | 20741 | CA | THR | H | 50 | 42.612 | 51.686 | 54.291 | 1.00 | 80.24 | 6 |
| ATOM | 20742 | CB | THR | H | 50 | 43.688 | 51.772 | 53.168 | 1.00 | 161.47 | 6 |
| ATOM | 20743 | OG1 | THR | H | 50 | 44.432 | 52.990 | 53.304 | 1.00 | 162.18 | 8 |
| ATOM | 20744 | CG2 | THR | H | 50 | 43.036 | 51.753 | 51.791 | 1.00 | 161.65 | 6 |
| ATOM | 20745 | C | THR | H | 50 | 41.773 | 50.420 | 54.073 | 1.00 | 80.46 | 6 |
| ATOM | 20746 | O | THR | H | 50 | 42.195 | 49.319 | 54.424 | 1.00 | 80.69 | 8 |
| ATOM | 20747 | N | ALA | H | 51 | 40.587 | 50.608 | 53.493 | 1.00 | 170.99 | 7 |
| ATOM | 20748 | CA | ALA | H | 51 | 39.633 | 49.540 | 53.179 | 1.00 | 171.40 | 6 |
| ATOM | 20749 | CB | ALA | H | 51 | 39.772 | 49.144 | 51.708 | 1.00 | 135.49 | 6 |
| ATOM | 20750 | C | ALA | H | 51 | 39.688 | 48.288 | 54.055 | 1.00 | 171.67 | 6 |
| ATOM | 20751 | O | ALA | H | 51 | 38.722 | 47.961 | 54.746 | 1.00 | 171.50 | 8 |
| ATOM | 20752 | N | ALA | H | 52 | 40.813 | 47.584 | 54.022 | 1.00 | 123.85 | 7 |
| ATOM | 20753 | CA | ALA | H | 52 | 40.963 | 46.368 | 54.806 | 1.00 | 124.49 | 6 |
| ATOM | 20754 | CB | ALA | H | 52 | 41.502 | 45.261 | 53.918 | 1.00 | 35.38 | 6 |
| ATOM | 20755 | C | ALA | H | 52 | 41.843 | 46.510 | 56.049 | 1.00 | 125.38 | 6 |
| ATOM | 20756 | O | ALA | H | 52 | 42.619 | 45.610 | 56.363 | 1.00 | 126.00 | 8 |
| ATOM | 20757 | N | GLY | H | 53 | 41.729 | 47.631 | 56.754 | 1.00 | 116.12 | 7 |
| ATOM | 20758 | CA | GLY | H | 53 | 42.521 | 47.817 | 57.959 | 1.00 | 115.99 | 6 |
| ATOM | 20759 | C | GLY | H | 53 | 43.816 | 48.601 | 57.825 | 1.00 | 115.70 | 6 |
| ATOM | 20760 | O | GLY | H | 53 | 43.972 | 49.434 | 56.934 | 1.00 | 116.78 | 8 |
| ATOM | 20761 | N | LEU | H | 54 | 44.749 | 48.312 | 58.726 | 1.00 | 60.84 | 7 |
| ATOM | 20762 | CA | LEU | H | 54 | 46.052 | 48.971 | 58.782 | 1.00 | 60.49 | 6 |
| ATOM | 20763 | CB | LEU | H | 54 | 46.635 | 48.814 | 60.186 | 1.00 | 79.09 | 6 |
| ATOM | 20764 | CG | LEU | H | 54 | 46.064 | 49.648 | 61.336 | 1.00 | 78.68 | 6 |
| ATOM | 20765 | CD1 | LEU | H | 54 | 44.560 | 49.858 | 61.201 | 1.00 | 78.11 | 6 |
| ATOM | 20766 | CD2 | LEU | H | 54 | 46.405 | 48.940 | 62.635 | 1.00 | 78.38 | 6 |
| ATOM | 20767 | C | LEU | H | 54 | 47.069 | 48.469 | 57.762 | 1.00 | 60.34 | 6 |
| ATOM | 20768 | O | LEU | H | 54 | 46.745 | 47.670 | 56.891 | 1.00 | 60.52 | 8 |
| ATOM | 20769 | N | ALA | H | 55 | 48.304 | 48.948 | 57.889 | 1.00 | 77.77 | 7 |
| ATOM | 20770 | CA | ALA | H | 55 | 49.411 | 48.574 | 57.004 | 1.00 | 78.07 | 6 |
| ATOM | 20771 | CB | ALA | H | 55 | 48.928 | 48.452 | 55.557 | 1.00 | 81.78 | 6 |
| ATOM | 20772 | C | ALA | H | 55 | 50.490 | 49.648 | 57.100 | 1.00 | 78.45 | 6 |
| ATOM | 20773 | O | ALA | H | 55 | 50.365 | 50.710 | 56.493 | 1.00 | 78.70 | 8 |
| ATOM | 20774 | N | ALA | H | 56 | 51.541 | 49.374 | 57.868 | 1.00 | 173.90 | 7 |
| ATOM | 20775 | CA | ALA | H | 56 | 52.640 | 50.324 | 58.050 | 1.00 | 174.29 | 6 |
| ATOM | 20776 | CB | ALA | H | 56 | 53.917 | 49.573 | 58.392 | 1.00 | 124.91 | 6 |
| ATOM | 20777 | C | ALA | H | 56 | 52.854 | 51.176 | 56.801 | 1.00 | 174.77 | 6 |
| ATOM | 20778 | O | ALA | H | 56 | 52.824 | 50.658 | 55.684 | 1.00 | 175.59 | 8 |
| ATOM | 20779 | N | ASP | H | 57 | 53.073 | 52.478 | 56.982 | 1.00 | 71.54 | 7 |
| ATOM | 20780 | CA | ASP | H | 57 | 53.272 | 53.358 | 55.830 | 1.00 | 70.65 | 6 |
| ATOM | 20781 | CB | ASP | H | 57 | 52.810 | 54.792 | 56.144 | 1.00 | 164.99 | 6 |
| ATOM | 20782 | CG | ASP | H | 57 | 53.538 | 55.405 | 57.321 | 1.00 | 166.34 | 6 |
| ATOM | 20783 | OD1 | ASP | H | 57 | 54.461 | 54.755 | 57.856 | 1.00 | 164.60 | 8 |
| ATOM | 20784 | OD2 | ASP | H | 57 | 53.189 | 56.542 | 57.706 | 1.00 | 168.33 | 8 |
| ATOM | 20785 | C | ASP | H | 57 | 54.702 | 53.370 | 55.286 | 1.00 | 69.56 | 6 |
| ATOM | 20786 | O | ASP | H | 57 | 55.667 | 53.530 | 56.029 | 1.00 | 70.12 | 8 |
| ATOM | 20787 | N | PRO | H | 58 | 54.846 | 53.197 | 53.965 | 1.00 | 175.82 | 7 |
| ATOM | 20788 | CD | PRO | H | 58 | 53.734 | 53.009 | 53.018 | 1.00 | 126.29 | 6 |
| ATOM | 20789 | CA | PRO | H | 58 | 56.132 | 53.174 | 53.261 | 1.00 | 175.53 | 6 |
| ATOM | 20790 | CB | PRO | H | 58 | 55.714 | 53.109 | 51.796 | 1.00 | 127.10 | 6 |
| ATOM | 20791 | CG | PRO | H | 58 | 54.419 | 52.358 | 51.856 | 1.00 | 127.14 | 6 |
| ATOM | 20792 | C | PRO | H | 58 | 57.033 | 54.375 | 53.549 | 1.00 | 174.24 | 6 |
| ATOM | 20793 | O | PRO | H | 58 | 58.243 | 54.321 | 53.325 | 1.00 | 174.62 | 8 |
| ATOM | 20794 | N | ASN | H | 59 | 56.438 | 55.455 | 54.041 | 1.00 | 50.78 | 7 |
| ATOM | 20795 | CA | ASN | H | 59 | 57.180 | 56.672 | 54.356 | 1.00 | 48.68 | 6 |
| ATOM | 20796 | CB | ASN | H | 59 | 56.226 | 57.742 | 54.893 | 1.00 | 106.68 | 6 |
| ATOM | 20797 | CG | ASN | H | 59 | 56.842 | 59.120 | 54.901 | 1.00 | 108.47 | 6 |
| ATOM | 20798 | OD1 | ASN | H | 59 | 57.139 | 59.683 | 53.852 | 1.00 | 109.20 | 8 |
| ATOM | 20799 | ND2 | ASN | H | 59 | 57.042 | 59.669 | 56.084 | 1.00 | 108.55 | 7 |
| ATOM | 20800 | C | ASN | H | 59 | 58.237 | 56.349 | 55.402 | 1.00 | 46.84 | 6 |
| ATOM | 20801 | O | ASN | H | 59 | 57.916 | 56.157 | 56.575 | 1.00 | 47.13 | 8 |
| ATOM | 20802 | N | ALA | H | 60 | 59.495 | 56.299 | 54.970 | 1.00 | 93.90 | 7 |
| ATOM | 20803 | CA | ALA | H | 60 | 60.618 | 55.975 | 55.850 | 1.00 | 90.64 | 6 |
| ATOM | 20804 | CB | ALA | H | 60 | 61.875 | 55.758 | 55.005 | 1.00 | 49.58 | 6 |
| ATOM | 20805 | C | ALA | H | 60 | 60.886 | 56.995 | 56.974 | 1.00 | 88.46 | 6 |
| ATOM | 20806 | O | ALA | H | 60 | 61.051 | 56.614 | 58.138 | 1.00 | 88.36 | 8 |
| ATOM | 20807 | N | VAL | H | 61 | 60.936 | 58.280 | 56.629 | 1.00 | 51.76 | 7 |
| ATOM | 20808 | CA | VAL | H | 61 | 61.156 | 59.337 | 57.622 | 1.00 | 48.32 | 6 |
| ATOM | 20809 | CB | VAL | H | 61 | 60.849 | 60.739 | 57.044 | 1.00 | 27.29 | 6 |
| ATOM | 20810 | CG1 | VAL | H | 61 | 62.109 | 61.396 | 56.500 | 1.00 | 26.01 | 6 |
| ATOM | 20811 | CG2 | VAL | H | 61 | 59.811 | 60.604 | 55.957 | 1.00 | 26.76 | 6 |
| ATOM | 20812 | C | VAL | H | 61 | 60.219 | 59.146 | 58.813 | 1.00 | 46.60 | 6 |
| ATOM | 20813 | O | VAL | H | 61 | 60.590 | 59.401 | 59.963 | 1.00 | 46.66 | 8 |
| ATOM | 20814 | N | THR | H | 62 | 58.996 | 58.707 | 58.524 | 1.00 | 32.73 | 7 |
| ATOM | 20815 | CA | THR | H | 62 | 57.996 | 58.512 | 59.564 | 1.00 | 30.58 | 6 |
| ATOM | 20816 | CB | THR | H | 62 | 56.754 | 57.746 | 59.039 | 1.00 | 91.22 | 6 |
| ATOM | 20817 | OG1 | THR | H | 62 | 56.239 | 58.408 | 57.877 | 1.00 | 92.33 | 8 |
| ATOM | 20818 | CG2 | THR | H | 62 | 55.655 | 57.705 | 60.111 | 1.00 | 92.71 | 6 |
| ATOM | 20819 | C | THR | H | 62 | 58.598 | 57.752 | 60.730 | 1.00 | 28.54 | 6 |
| ATOM | 20820 | O | THR | H | 62 | 58.818 | 58.321 | 61.798 | 1.00 | 27.90 | 8 |
| ATOM | 20821 | N | TRP | H | 63 | 58.878 | 56.470 | 60.518 | 1.00 | 47.29 | 7 |
| ATOM | 20822 | CA | TRP | H | 63 | 59.441 | 55.649 | 61.571 | 1.00 | 47.12 | 6 |
| ATOM | 20823 | CB | TRP | H | 63 | 60.002 | 54.369 | 60.998 | 1.00 | 55.89 | 6 |
| ATOM | 20824 | CG | TRP | H | 63 | 58.961 | 53.500 | 60.430 | 1.00 | 56.38 | 6 |
| ATOM | 20825 | CD2 | TRP | H | 63 | 58.571 | 52.217 | 60.912 | 1.00 | 56.28 | 6 |
| ATOM | 20826 | CE2 | TRP | H | 63 | 57.597 | 51.718 | 60.028 | 1.00 | 56.76 | 6 |
| ATOM | 20827 | CE3 | TRP | H | 63 | 58.953 | 51.435 | 62.010 | 1.00 | 56.09 | 6 |
| ATOM | 20828 | CD1 | TRP | H | 63 | 58.224 | 53.730 | 59.313 | 1.00 | 57.16 | 6 |
| ATOM | 20829 | NE1 | TRP | H | 63 | 57.403 | 52.664 | 59.059 | 1.00 | 57.27 | 7 |
| ATOM | 20830 | CZ2 | TRP | H | 63 | 56.999 | 50.471 | 60.201 | 1.00 | 57.29 | 6 |
| ATOM | 20831 | CZ3 | TRP | H | 63 | 58.358 | 50.197 | 62.184 | 1.00 | 56.08 | 6 |
| ATOM | 20832 | CH2 | TRP | H | 63 | 57.392 | 49.726 | 61.283 | 1.00 | 56.89 | 6 |
| ATOM | 20833 | C | TRP | H | 63 | 60.536 | 56.409 | 62.283 | 1.00 | 47.29 | 6 |
| ATOM | 20834 | O | TRP | H | 63 | 60.435 | 56.678 | 63.482 | 1.00 | 46.74 | 8 |
| ATOM | 20835 | N | ALA | H | 64 | 61.570 | 56.773 | 61.533 | 1.00 | 43.86 | 7 |
| ATOM | 20836 | CA | ALA | H | 64 | 62.696 | 57.500 | 62.098 | 1.00 | 44.72 | 6 |
| ATOM | 20837 | CB | ALA | H | 64 | 63.431 | 58.249 | 61.004 | 1.00 | 13.87 | 6 |
| ATOM | 20838 | C | ALA | H | 64 | 62.248 | 58.464 | 63.183 | 1.00 | 45.71 | 6 |
| ATOM | 20839 | O | ALA | H | 64 | 62.508 | 58.239 | 64.361 | 1.00 | 46.59 | 8 |
| ATOM | 20840 | N | MET | H | 65 | 61.563 | 59.530 | 62.792 | 1.00 | 63.70 | 7 |
| ATOM | 20841 | CA | MET | H | 65 | 61.116 | 60.505 | 63.770 | 1.00 | 63.47 | 6 |
| ATOM | 20842 | CB | MET | H | 65 | 60.134 | 61.486 | 63.133 | 1.00 | 47.39 | 6 |
| ATOM | 20843 | CG | MET | H | 65 | 60.811 | 62.505 | 62.230 | 1.00 | 45.91 | 6 |
| ATOM | 20844 | SD | MET | H | 65 | 59.660 | 63.619 | 61.419 | 1.00 | 44.79 | 16 |
| ATOM | 20845 | CE | MET | H | 65 | 59.613 | 62.879 | 59.784 | 1.00 | 42.87 | 6 |
| ATOM | 20846 | C | MET | H | 65 | 60.495 | 59.832 | 64.985 | 1.00 | 64.92 | 6 |
| ATOM | 20847 | O | MET | H | 65 | 60.580 | 60.354 | 66.093 | 1.00 | 65.17 | 8 |
| ATOM | 20848 | N | LYS | H | 66 | 59.893 | 58.663 | 64.788 | 1.00 | 59.31 | 7 |
| ATOM | 20849 | CA | LYS | H | 66 | 59.270 | 57.944 | 65.894 | 1.00 | 61.28 | 6 |
| ATOM | 20850 | CB | LYS | H | 66 | 58.046 | 57.169 | 65.399 | 1.00 | 151.34 | 6 |
| ATOM | 20851 | CG | LYS | H | 66 | 56.948 | 58.054 | 64.827 | 1.00 | 153.48 | 6 |
| ATOM | 20852 | CD | LYS | H | 66 | 55.742 | 57.243 | 64.380 | 1.00 | 154.12 | 6 |
| ATOM | 20853 | CE | LYS | H | 66 | 54.685 | 58.137 | 63.748 | 1.00 | 154.76 | 6 |
| ATOM | 20854 | NZ | LYS | H | 66 | 53.501 | 57.379 | 63.263 | 1.00 | 156.89 | 7 |
| ATOM | 20855 | C | LYS | H | 66 | 60.258 | 56.990 | 66.551 | 1.00 | 61.91 | 6 |
| ATOM | 20856 | O | LYS | H | 66 | 60.190 | 56.727 | 67.752 | 1.00 | 62.08 | 8 |
| ATOM | 20857 | N | GLU | H | 67 | 61.180 | 56.483 | 65.747 | 1.00 | 103.46 | 7 |
| ATOM | 20858 | CA | GLU | H | 67 | 62.203 | 55.552 | 66.198 | 1.00 | 104.40 | 6 |
| ATOM | 20859 | CB | GLU | H | 67 | 62.722 | 54.786 | 64.975 | 1.00 | 70.74 | 6 |
| ATOM | 20860 | CG | GLU | H | 67 | 63.845 | 53.800 | 65.192 | 1.00 | 71.76 | 6 |
| ATOM | 20861 | CD | GLU | H | 67 | 64.382 | 53.273 | 63.870 | 1.00 | 73.23 | 6 |
| ATOM | 20862 | OE1 | GLU | H | 67 | 63.738 | 52.419 | 63.225 | 1.00 | 72.69 | 8 |
| ATOM | 20863 | OE2 | GLU | H | 67 | 65.458 | 53.735 | 63.465 | 1.00 | 75.45 | 8 |
| ATOM | 20864 | C | GLU | H | 67 | 63.327 | 56.337 | 66.879 | 1.00 | 104.13 | 6 |
| ATOM | 20865 | O | GLU | H | 67 | 64.159 | 55.767 | 67.588 | 1.00 | 104.77 | 8 |
| ATOM | 20866 | N | LEU | H | 68 | 63.325 | 57.652 | 66.669 | 1.00 | 56.33 | 7 |
| ATOM | 20867 | CA | LEU | H | 68 | 64.341 | 58.545 | 67.230 | 1.00 | 56.02 | 6 |
| ATOM | 20868 | CB | LEU | H | 68 | 64.668 | 59.652 | 66.212 | 1.00 | 69.52 | 6 |
| ATOM | 20869 | CG | LEU | H | 68 | 65.819 | 60.625 | 66.479 | 1.00 | 70.25 | 6 |
| ATOM | 20870 | CD1 | LEU | H | 68 | 67.132 | 59.886 | 66.657 | 1.00 | 70.86 | 6 |
| ATOM | 20871 | CD2 | LEU | H | 68 | 65.928 | 61.566 | 65.313 | 1.00 | 69.91 | 6 |
| ATOM | 20872 | C | LEU | H | 68 | 63.876 | 59.159 | 68.554 | 1.00 | 55.23 | 6 |
| ATOM | 20873 | O | LEU | H | 68 | 64.683 | 59.520 | 69.416 | 1.00 | 54.45 | 8 |
| ATOM | 20874 | N | LEU | H | 69 | 62.565 | 59.269 | 68.709 | 1.00 | 33.13 | 7 |
| ATOM | 20875 | CA | LEU | H | 69 | 62.004 | 59.831 | 69.915 | 1.00 | 33.85 | 6 |
| ATOM | 20876 | CB | LEU | H | 69 | 60.554 | 60.216 | 69.683 | 1.00 | 55.55 | 6 |
| ATOM | 20877 | CG | LEU | H | 69 | 60.003 | 61.108 | 70.785 | 1.00 | 56.38 | 6 |
| ATOM | 20878 | CD1 | LEU | H | 69 | 60.640 | 62.486 | 70.648 | 1.00 | 56.76 | 6 |
| ATOM | 20879 | CD2 | LEU | H | 69 | 58.492 | 61.190 | 70.683 | 1.00 | 56.14 | 6 |
| ATOM | 20880 | C | LEU | H | 69 | 62.067 | 58.742 | 70.951 | 1.00 | 35.09 | 6 |
| ATOM | 20881 | O | LEU | H | 69 | 61.862 | 58.985 | 72.143 | 1.00 | 35.06 | 8 |
| ATOM | 20882 | N | THR | H | 70 | 62.351 | 57.535 | 70.461 | 1.00 | 65.83 | 7 |
| ATOM | 20883 | CA | THR | H | 70 | 62.451 | 56.313 | 71.259 | 1.00 | 66.19 | 6 |
| ATOM | 20884 | CB | THR | H | 70 | 62.533 | 55.076 | 70.340 | 1.00 | 57.53 | 6 |
| ATOM | 20885 | OG1 | THR | H | 70 | 61.577 | 55.199 | 69.279 | 1.00 | 56.80 | 8 |
| ATOM | 20886 | CG2 | THR | H | 70 | 62.238 | 53.812 | 71.130 | 1.00 | 57.67 | 6 |
| ATOM | 20887 | C | THR | H | 70 | 63.657 | 56.290 | 72.203 | 1.00 | 67.12 | 6 |
| ATOM | 20888 | O | THR | H | 70 | 63.702 | 57.028 | 73.181 | 1.00 | 66.97 | 8 |
| ATOM | 20889 | N | GLY | H | 71 | 64.635 | 55.442 | 71.916 | 1.00 | 87.16 | 7 |
| ATOM | 20890 | CA | GLY | H | 71 | 65.794 | 55.377 | 72.786 | 1.00 | 89.30 | 6 |
| ATOM | 20891 | C | GLY | H | 71 | 67.070 | 54.978 | 72.076 | 1.00 | 91.08 | 6 |
| ATOM | 20892 | O | GLY | H | 71 | 68.146 | 55.515 | 72.350 | 1.00 | 90.90 | 8 |
| ATOM | 20893 | N | ARG | H | 72 | 66.953 | 54.033 | 71.154 | 1.00 | 70.67 | 7 |
| ATOM | 20894 | CA | ARG | H | 72 | 68.107 | 53.562 | 70.414 | 1.00 | 72.13 | 6 |
| ATOM | 20895 | CB | ARG | H | 72 | 67.709 | 52.357 | 69.558 | 1.00 | 115.78 | 6 |
| ATOM | 20896 | CG | ARG | H | 72 | 67.104 | 51.217 | 70.380 | 1.00 | 116.51 | 6 |
| ATOM | 20897 | CD | ARG | H | 72 | 68.086 | 50.737 | 71.446 | 1.00 | 117.76 | 6 |
| ATOM | 20898 | NE | ARG | H | 72 | 67.431 | 50.255 | 72.663 | 1.00 | 116.94 | 7 |
| ATOM | 20899 | CZ | ARG | H | 72 | 68.080 | 49.898 | 73.769 | 1.00 | 115.88 | 6 |
| ATOM | 20900 | NH1 | ARG | H | 72 | 69.403 | 49.965 | 73.817 | 1.00 | 115.31 | 7 |
| ATOM | 20901 | NH2 | ARG | H | 72 | 67.407 | 49.481 | 74.834 | 1.00 | 116.05 | 7 |
| ATOM | 20902 | C | ARG | H | 72 | 68.698 | 54.668 | 69.553 | 1.00 | 72.71 | 6 |
| ATOM | 20903 | O | ARG | H | 72 | 68.372 | 55.842 | 69.719 | 1.00 | 73.18 | 8 |
| ATOM | 20904 | N | LEU | H | 73 | 69.581 | 54.283 | 68.641 | 1.00 | 78.66 | 7 |
| ATOM | 20905 | CA | LEU | H | 73 | 70.234 | 55.231 | 67.748 | 1.00 | 79.90 | 6 |
| ATOM | 20906 | CB | LEU | H | 73 | 69.210 | 56.225 | 67.179 | 1.00 | 62.87 | 6 |
| ATOM | 20907 | CG | LEU | H | 73 | 67.857 | 55.752 | 66.639 | 1.00 | 63.54 | 6 |
| ATOM | 20908 | CD1 | LEU | H | 73 | 67.295 | 56.824 | 65.722 | 1.00 | 62.90 | 6 |
| ATOM | 20909 | CD2 | LEU | H | 73 | 68.014 | 54.458 | 65.868 | 1.00 | 63.93 | 6 |
| ATOM | 20910 | C | LEU | H | 73 | 71.342 | 56.013 | 68.460 | 1.00 | 81.03 | 6 |
| ATOM | 20911 | O | LEU | H | 73 | 71.413 | 56.040 | 69.686 | 1.00 | 81.93 | 8 |
| ATOM | 20912 | N | ALA | H | 74 | 72.199 | 56.652 | 67.673 | 1.00 | 73.20 | 7 |
| ATOM | 20913 | CA | ALA | H | 74 | 73.294 | 57.463 | 68.195 | 1.00 | 74.49 | 6 |
| ATOM | 20914 | CB | ALA | H | 74 | 74.435 | 56.571 | 68.633 | 1.00 | 131.27 | 6 |
| ATOM | 20915 | C | ALA | H | 74 | 73.750 | 58.410 | 67.077 | 1.00 | 75.22 | 6 |
| ATOM | 20916 | O | ALA | H | 74 | 73.253 | 58.310 | 65.953 | 1.00 | 75.67 | 8 |
| ATOM | 20917 | N | PHE | H | 75 | 74.681 | 59.321 | 67.364 | 1.00 | 51.42 | 7 |
| ATOM | 20918 | CA | PHE | H | 75 | 75.147 | 60.249 | 66.339 | 1.00 | 52.35 | 6 |
| ATOM | 20919 | CB | PHE | H | 75 | 73.970 | 61.078 | 65.825 | 1.00 | 111.18 | 6 |
| ATOM | 20920 | CG | PHE | H | 75 | 72.916 | 61.365 | 66.864 | 1.00 | 113.16 | 6 |
| ATOM | 20921 | CD1 | PHE | H | 75 | 73.256 | 61.600 | 68.193 | 1.00 | 114.86 | 6 |
| ATOM | 20922 | CD2 | PHE | H | 75 | 71.581 | 61.473 | 66.488 | 1.00 | 113.45 | 6 |
| ATOM | 20923 | CE1 | PHE | H | 75 | 72.281 | 61.946 | 69.128 | 1.00 | 115.47 | 6 |
| ATOM | 20924 | CE2 | PHE | H | 75 | 70.603 | 61.818 | 67.411 | 1.00 | 114.47 | 6 |
| ATOM | 20925 | CZ | PHE | H | 75 | 70.954 | 62.057 | 68.735 | 1.00 | 115.44 | 6 |
| ATOM | 20926 | C | PHE | H | 75 | 76.265 | 61.191 | 66.774 | 1.00 | 52.45 | 6 |
| ATOM | 20927 | O | PHE | H | 75 | 76.148 | 61.855 | 67.794 | 1.00 | 53.14 | 8 |
| ATOM | 20928 | N | GLY | H | 76 | 77.327 | 61.264 | 65.971 | 1.00 | 70.31 | 7 |
| ATOM | 20929 | CA | GLY | H | 76 | 78.469 | 62.126 | 66.259 | 1.00 | 70.79 | 6 |
| ATOM | 20930 | C | GLY | H | 76 | 79.666 | 61.701 | 65.419 | 1.00 | 71.08 | 6 |
| ATOM | 20931 | O | GLY | H | 76 | 79.619 | 61.775 | 64.193 | 1.00 | 71.12 | 8 |
| ATOM | 20932 | N | GLU | H | 77 | 80.751 | 61.279 | 66.059 | 1.00 | 77.05 | 7 |
| ATOM | 20933 | CA | GLU | H | 77 | 81.915 | 60.797 | 65.319 | 1.00 | 78.21 | 6 |
| ATOM | 20934 | CB | GLU | H | 77 | 83.201 | 61.459 | 65.814 | 1.00 | 92.11 | 6 |
| ATOM | 20935 | CG | GLU | H | 77 | 84.450 | 60.998 | 65.072 | 1.00 | 93.17 | 6 |
| ATOM | 20936 | CD | GLU | H | 77 | 84.416 | 61.299 | 63.570 | 1.00 | 94.00 | 6 |
| ATOM | 20937 | OE1 | GLU | H | 77 | 83.386 | 61.034 | 62.910 | 1.00 | 94.23 | 8 |
| ATOM | 20938 | OE2 | GLU | H | 77 | 85.437 | 61.788 | 63.041 | 1.00 | 94.19 | 8 |
| ATOM | 20939 | C | GLU | H | 77 | 81.953 | 59.289 | 65.591 | 1.00 | 79.04 | 6 |
| ATOM | 20940 | O | GLU | H | 77 | 82.931 | 58.592 | 65.293 | 1.00 | 79.54 | 8 |
| ATOM | 20941 | N | ASN | H | 78 | 80.848 | 58.807 | 66.154 | 1.00 | 125.12 | 7 |
| ATOM | 20942 | CA | ASN | H | 78 | 80.649 | 57.411 | 66.501 | 1.00 | 125.42 | 6 |
| ATOM | 20943 | CB | ASN | H | 78 | 79.417 | 57.293 | 67.401 | 1.00 | 204.16 | 6 |
| ATOM | 20944 | CG | ASN | H | 78 | 78.783 | 55.925 | 67.345 | 1.00 | 204.44 | 6 |
| ATOM | 20945 | OD1 | ASN | H | 78 | 78.084 | 55.601 | 66.388 | 1.00 | 202.88 | 8 |
| ATOM | 20946 | ND2 | ASN | H | 78 | 79.027 | 55.109 | 68.365 | 1.00 | 205.31 | 7 |
| ATOM | 20947 | C | ASN | H | 78 | 80.516 | 56.487 | 65.300 | 1.00 | 126.30 | 6 |
| ATOM | 20948 | O | ASN | H | 78 | 80.052 | 56.886 | 64.236 | 1.00 | 127.62 | 8 |
| ATOM | 20949 | N | LEU | H | 79 | 80.934 | 55.243 | 65.511 | 1.00 | 110.62 | 7 |
| ATOM | 20950 | CA | LEU | H | 79 | 80.917 | 54.179 | 64.510 | 1.00 | 111.17 | 6 |
| ATOM | 20951 | CB | LEU | H | 79 | 79.576 | 54.133 | 63.766 | 1.00 | 132.86 | 6 |
| ATOM | 20952 | CG | LEU | H | 79 | 78.371 | 53.654 | 64.588 | 1.00 | 132.88 | 6 |
| ATOM | 20953 | CD1 | LEU | H | 79 | 77.438 | 52.894 | 63.681 | 1.00 | 133.18 | 6 |
| ATOM | 20954 | CD2 | LEU | H | 79 | 78.801 | 52.732 | 65.731 | 1.00 | 132.83 | 6 |
| ATOM | 20955 | C | LEU | H | 79 | 82.072 | 54.277 | 63.516 | 1.00 | 111.11 | 6 |
| ATOM | 20956 | O | LEU | H | 79 | 82.439 | 53.292 | 62.891 | 1.00 | 111.12 | 8 |
| ATOM | 20957 | N | ALA | H | 80 | 82.638 | 55.473 | 63.403 | 1.00 | 122.68 | 7 |
| ATOM | 20958 | CA | ALA | H | 80 | 83.764 | 55.783 | 62.527 | 1.00 | 123.16 | 6 |
| ATOM | 20959 | CB | ALA | H | 80 | 85.076 | 55.429 | 63.239 | 1.00 | 104.62 | 6 |
| ATOM | 20960 | C | ALA | H | 80 | 83.764 | 55.228 | 61.094 | 1.00 | 123.32 | 6 |
| ATOM | 20961 | O | ALA | H | 80 | 83.403 | 55.942 | 60.163 | 1.00 | 123.48 | 8 |
| ATOM | 20962 | N | PRO | H | 81 | 84.168 | 53.958 | 60.888 | 1.00 | 118.44 | 7 |
| ATOM | 20963 | CD | PRO | H | 81 | 84.672 | 52.906 | 61.792 | 1.00 | 173.71 | 6 |
| ATOM | 20964 | CA | PRO | H | 81 | 84.159 | 53.477 | 59.499 | 1.00 | 118.82 | 6 |
| ATOM | 20965 | CB | PRO | H | 81 | 84.817 | 52.101 | 59.606 | 1.00 | 174.43 | 6 |
| ATOM | 20966 | CG | PRO | H | 81 | 84.440 | 51.658 | 60.987 | 1.00 | 174.33 | 6 |
| ATOM | 20967 | C | PRO | H | 81 | 82.797 | 53.423 | 58.813 | 1.00 | 118.74 | 6 |
| ATOM | 20968 | O | PRO | H | 81 | 82.007 | 52.506 | 59.033 | 1.00 | 118.56 | 8 |
| ATOM | 20969 | N | ALA | H | 82 | 82.532 | 54.417 | 57.976 | 1.00 | 90.20 | 7 |
| ATOM | 20970 | CA | ALA | H | 82 | 81.279 | 54.473 | 57.242 | 1.00 | 90.98 | 6 |
| ATOM | 20971 | CB | ALA | H | 82 | 81.275 | 55.693 | 56.310 | 1.00 | 18.02 | 6 |
| ATOM | 20972 | C | ALA | H | 82 | 81.218 | 53.176 | 56.445 | 1.00 | 91.69 | 6 |
| ATOM | 20973 | O | ALA | H | 82 | 80.147 | 52.632 | 56.167 | 1.00 | 91.55 | 8 |
| ATOM | 20974 | N | ASP | H | 83 | 82.400 | 52.683 | 56.094 | 1.00 | 208.87 | 7 |
| ATOM | 20975 | CA | ASP | H | 83 | 82.533 | 51.445 | 55.345 | 1.00 | 208.87 | 6 |
| ATOM | 20976 | CB | ASP | H | 83 | 84.012 | 51.162 | 55.069 | 1.00 | 201.99 | 6 |
| ATOM | 20977 | CG | ASP | H | 83 | 84.698 | 52.304 | 54.344 | 1.00 | 202.55 | 6 |
| ATOM | 20978 | OD1 | ASP | H | 83 | 84.393 | 52.526 | 53.153 | 1.00 | 203.98 | 8 |
| ATOM | 20979 | OD2 | ASP | H | 83 | 85.537 | 52.980 | 54.973 | 1.00 | 201.53 | 8 |
| ATOM | 20980 | C | ASP | H | 83 | 81.948 | 50.317 | 56.184 | 1.00 | 208.87 | 6 |
| ATOM | 20981 | O | ASP | H | 83 | 80.969 | 49.682 | 55.796 | 1.00 | 208.87 | 8 |
| ATOM | 20982 | N | ARG | H | 84 | 82.554 | 50.087 | 57.343 | 1.00 | 91.02 | 7 |
| ATOM | 20983 | CA | ARG | H | 84 | 82.112 | 49.041 | 58.252 | 1.00 | 90.84 | 6 |
| ATOM | 20984 | CB | ARG | H | 84 | 83.156 | 48.859 | 59.342 | 1.00 | 129.70 | 6 |
| ATOM | 20985 | CG | ARG | H | 84 | 84.554 | 48.797 | 58.770 | 1.00 | 130.16 | 6 |
| ATOM | 20986 | CD | ARG | H | 84 | 85.609 | 48.783 | 59.846 | 1.00 | 130.44 | 6 |
| ATOM | 20987 | NE | ARG | H | 84 | 86.939 | 48.922 | 59.268 | 1.00 | 130.69 | 7 |
| ATOM | 20988 | CZ | ARG | H | 84 | 88.046 | 49.088 | 59.980 | 1.00 | 131.22 | 6 |
| ATOM | 20989 | NH1 | ARG | H | 84 | 87.981 | 49.133 | 61.303 | 1.00 | 131.11 | 7 |
| ATOM | 20990 | NH2 | ARG | H | 84 | 89.217 | 49.217 | 59.370 | 1.00 | 132.39 | 7 |
| ATOM | 20991 | C | ARG | H | 84 | 80.766 | 49.421 | 58.859 | 1.00 | 90.69 | 6 |
| ATOM | 20992 | O | ARG | H | 84 | 80.185 | 48.674 | 59.655 | 1.00 | 90.08 | 8 |
| ATOM | 20993 | N | LEU | H | 85 | 80.286 | 50.599 | 58.473 | 1.00 | 94.70 | 7 |
| ATOM | 20994 | CA | LEU | H | 85 | 79.012 | 51.111 | 58.945 | 1.00 | 94.79 | 6 |
| ATOM | 20995 | CB | LEU | H | 85 | 78.971 | 52.632 | 58.794 | 1.00 | 58.74 | 6 |
| ATOM | 20996 | CG | LEU | H | 85 | 77.645 | 53.320 | 59.129 | 1.00 | 58.15 | 6 |
| ATOM | 20997 | CD1 | LEU | H | 85 | 77.346 | 53.162 | 60.610 | 1.00 | 58.00 | 6 |
| ATOM | 20998 | CD2 | LEU | H | 85 | 77.711 | 54.797 | 58.727 | 1.00 | 58.19 | 6 |
| ATOM | 20999 | C | LEU | H | 85 | 77.923 | 50.466 | 58.102 | 1.00 | 95.17 | 6 |
| ATOM | 21000 | O | LEU | H | 85 | 76.769 | 50.367 | 58.522 | 1.00 | 95.24 | 8 |
| ATOM | 21001 | N | ALA | H | 86 | 78.309 | 50.026 | 56.908 | 1.00 | 162.60 | 7 |
| ATOM | 21002 | CA | ALA | H | 86 | 77.389 | 49.370 | 55.989 | 1.00 | 163.48 | 6 |
| ATOM | 21003 | CB | ALA | H | 86 | 77.351 | 50.113 | 54.661 | 1.00 | 148.76 | 6 |
| ATOM | 21004 | C | ALA | H | 86 | 77.845 | 47.934 | 55.773 | 1.00 | 163.77 | 6 |
| ATOM | 21005 | O | ALA | H | 86 | 77.064 | 47.075 | 55.367 | 1.00 | 163.80 | 8 |
| ATOM | 21006 | N | ALA | H | 87 | 79.120 | 47.681 | 56.037 | 1.00 | 104.44 | 7 |
| ATOM | 21007 | CA | ALA | H | 87 | 79.664 | 46.341 | 55.876 | 1.00 | 104.76 | 6 |
| ATOM | 21008 | CB | ALA | H | 87 | 81.168 | 46.348 | 56.136 | 1.00 | 143.84 | 6 |
| ATOM | 21009 | C | ALA | H | 87 | 78.956 | 45.442 | 56.878 | 1.00 | 104.89 | 6 |
| ATOM | 21010 | O | ALA | H | 87 | 78.645 | 44.284 | 56.594 | 1.00 | 104.28 | 8 |
| ATOM | 21011 | N | ALA | H | 88 | 78.701 | 46.000 | 58.055 | 1.00 | 80.31 | 7 |
| ATOM | 21012 | CA | ALA | H | 88 | 78.025 | 45.275 | 59.113 | 1.00 | 81.22 | 6 |
| ATOM | 21013 | CB | ALA | H | 88 | 78.063 | 46.092 | 60.402 | 1.00 | 112.51 | 6 |
| ATOM | 21014 | C | ALA | H | 88 | 76.582 | 45.004 | 58.683 | 1.00 | 81.94 | 6 |
| ATOM | 21015 | O | ALA | H | 88 | 75.892 | 44.153 | 59.261 | 1.00 | 82.24 | 8 |
| ATOM | 21016 | N | MET | H | 89 | 76.133 | 45.731 | 57.664 | 1.00 | 83.34 | 7 |
| ATOM | 21017 | CA | MET | H | 89 | 74.782 | 45.564 | 57.145 | 1.00 | 85.09 | 6 |
| ATOM | 21018 | CB | MET | H | 89 | 74.411 | 46.732 | 56.228 | 1.00 | 155.16 | 6 |
| ATOM | 21019 | CG | MET | H | 89 | 74.405 | 48.097 | 56.901 | 1.00 | 156.58 | 6 |
| ATOM | 21020 | SD | MET | H | 89 | 73.093 | 48.296 | 58.117 | 1.00 | 157.37 | 16 |
| ATOM | 21021 | CE | MET | H | 89 | 71.678 | 48.571 | 57.029 | 1.00 | 157.12 | 6 |
| ATOM | 21022 | C | MET | H | 89 | 74.712 | 44.255 | 56.360 | 1.00 | 85.71 | 6 |
| ATOM | 21023 | O | MET | H | 89 | 73.627 | 43.721 | 56.115 | 1.00 | 85.41 | 8 |
| ATOM | 21024 | N | GLU | H | 90 | 75.877 | 43.746 | 55.963 | 1.00 | 208.87 | 7 |
| ATOM | 21025 | CA | GLU | H | 90 | 75.950 | 42.493 | 55.217 | 1.00 | 208.87 | 6 |
| ATOM | 21026 | CB | GLU | H | 90 | 77.306 | 42.342 | 54.529 | 1.00 | 136.26 | 6 |
| ATOM | 21027 | CG | GLU | H | 90 | 77.457 | 41.014 | 53.798 | 1.00 | 136.21 | 6 |
| ATOM | 21028 | CD | GLU | H | 90 | 78.880 | 40.746 | 53.353 | 1.00 | 136.54 | 6 |
| ATOM | 21029 | OE1 | GLU | H | 90 | 79.460 | 41.610 | 52.666 | 1.00 | 137.49 | 8 |
| ATOM | 21030 | OE2 | GLU | H | 90 | 79.417 | 39.667 | 53.685 | 1.00 | 136.05 | 8 |
| ATOM | 21031 | C | GLU | H | 90 | 75.763 | 41.333 | 56.177 | 1.00 | 208.87 | 6 |
| ATOM | 21032 | O | GLU | H | 90 | 75.142 | 40.323 | 55.843 | 1.00 | 208.87 | 8 |
| ATOM | 21033 | N | ARG | H | 91 | 76.320 | 41.487 | 57.373 | 1.00 | 172.42 | 7 |
| ATOM | 21034 | CA | ARG | H | 91 | 76.222 | 40.463 | 58.396 | 1.00 | 172.92 | 6 |
| ATOM | 21035 | CB | ARG | H | 91 | 76.926 | 40.931 | 59.678 | 1.00 | 130.88 | 6 |
| ATOM | 21036 | CG | ARG | H | 91 | 77.084 | 39.848 | 60.750 | 1.00 | 131.31 | 6 |
| ATOM | 21037 | CD | ARG | H | 91 | 77.854 | 40.355 | 61.973 | 1.00 | 131.44 | 6 |
| ATOM | 21038 | NE | ARG | H | 91 | 77.774 | 39.432 | 63.107 | 1.00 | 131.71 | 7 |
| ATOM | 21039 | CZ | ARG | H | 91 | 78.253 | 39.698 | 64.319 | 1.00 | 131.97 | 6 |
| ATOM | 21040 | NH1 | ARG | H | 91 | 78.849 | 40.858 | 64.557 | 1.00 | 131.86 | 7 |
| ATOM | 21041 | NH2 | ARG | H | 91 | 78.127 | 38.813 | 65.299 | 1.00 | 132.07 | 7 |
| ATOM | 21042 | C | ARG | H | 91 | 74.746 | 40.185 | 58.658 | 1.00 | 173.25 | 6 |
| ATOM | 21043 | O | ARG | H | 91 | 74.389 | 39.146 | 59.207 | 1.00 | 173.60 | 8 |
| ATOM | 21044 | N | LEU | H | 92 | 73.892 | 41.117 | 58.241 | 1.00 | 114.03 | 7 |
| ATOM | 21045 | CA | LEU | H | 92 | 72.446 | 40.987 | 58.419 | 1.00 | 114.75 | 6 |
| ATOM | 21046 | CB | LEU | H | 92 | 72.007 | 41.810 | 59.635 | 1.00 | 107.98 | 6 |
| ATOM | 21047 | CG | LEU | H | 92 | 71.703 | 41.074 | 60.951 | 1.00 | 108.27 | 6 |
| ATOM | 21048 | CD1 | LEU | H | 92 | 72.560 | 39.839 | 61.125 | 1.00 | 108.03 | 6 |
| ATOM | 21049 | CD2 | LEU | H | 92 | 71.926 | 42.031 | 62.106 | 1.00 | 108.24 | 6 |
| ATOM | 21050 | C | LEU | H | 92 | 71.651 | 41.395 | 57.162 | 1.00 | 115.02 | 6 |
| ATOM | 21051 | O | LEU | H | 92 | 71.385 | 42.576 | 56.922 | 1.00 | 115.36 | 8 |
| ATOM | 21052 | N | ALA | H | 93 | 71.286 | 40.377 | 56.381 | 1.00 | 107.07 | 7 |
| ATOM | 21053 | CA | ALA | H | 93 | 70.538 | 40.474 | 55.121 | 1.00 | 107.38 | 6 |
| ATOM | 21054 | CB | ALA | H | 93 | 69.413 | 39.437 | 55.123 | 1.00 | 103.93 | 6 |
| ATOM | 21055 | C | ALA | H | 93 | 69.974 | 41.823 | 54.676 | 1.00 | 107.83 | 6 |
| ATOM | 21056 | O | ALA | H | 93 | 69.007 | 42.319 | 55.245 | 1.00 | 107.51 | 8 |
| ATOM | 21057 | N | PRO | H | 94 | 70.584 | 42.432 | 53.645 | 1.00 | 125.30 | 7 |
| ATOM | 21058 | CD | PRO | H | 94 | 72.012 | 42.193 | 53.357 | 1.00 | 176.05 | 6 |
| ATOM | 21059 | CA | PRO | H | 94 | 70.168 | 43.723 | 53.083 | 1.00 | 126.22 | 6 |
| ATOM | 21060 | CB | PRO | H | 94 | 71.460 | 44.514 | 53.110 | 1.00 | 176.57 | 6 |
| ATOM | 21061 | CG | PRO | H | 94 | 72.439 | 43.473 | 52.635 | 1.00 | 176.89 | 6 |
| ATOM | 21062 | C | PRO | H | 94 | 69.625 | 43.553 | 51.651 | 1.00 | 126.83 | 6 |
| ATOM | 21063 | O | PRO | H | 94 | 68.743 | 42.733 | 51.405 | 1.00 | 126.67 | 8 |
| ATOM | 21064 | N | ALA | H | 95 | 70.157 | 44.344 | 50.720 | 1.00 | 156.49 | 7 |
| ATOM | 21065 | CA | ALA | H | 95 | 69.785 | 44.281 | 49.304 | 1.00 | 157.56 | 6 |
| ATOM | 21066 | CB | ALA | H | 95 | 70.291 | 42.973 | 48.704 | 1.00 | 137.99 | 6 |
| ATOM | 21067 | C | ALA | H | 95 | 68.308 | 44.456 | 48.957 | 1.00 | 158.51 | 6 |
| ATOM | 21068 | O | ALA | H | 95 | 67.429 | 44.246 | 49.791 | 1.00 | 158.85 | 8 |
| ATOM | 21069 | N | ALA | H | 96 | 68.057 | 44.830 | 47.704 | 1.00 | 208.87 | 7 |
| ATOM | 21070 | CA | ALA | H | 96 | 66.704 | 45.037 | 47.194 | 1.00 | 208.87 | 6 |
| ATOM | 21071 | CB | ALA | H | 96 | 66.488 | 46.510 | 46.870 | 1.00 | 128.28 | 6 |
| ATOM | 21072 | C | ALA | H | 96 | 66.479 | 44.185 | 45.945 | 1.00 | 208.87 | 6 |
| ATOM | 21073 | O | ALA | H | 96 | 67.086 | 43.124 | 45.799 | 1.00 | 208.87 | 8 |
| ATOM | 21074 | N | ALA | H | 97 | 65.619 | 44.655 | 45.044 | 1.00 | 160.31 | 7 |
| ATOM | 21075 | CA | ALA | H | 97 | 65.313 | 43.912 | 43.820 | 1.00 | 160.01 | 6 |
| ATOM | 21076 | CB | ALA | H | 97 | 63.929 | 43.288 | 43.932 | 1.00 | 165.04 | 6 |
| ATOM | 21077 | C | ALA | H | 97 | 65.391 | 44.749 | 42.548 | 1.00 | 159.89 | 6 |
| ATOM | 21078 | O | ALA | H | 97 | 65.117 | 45.950 | 42.564 | 1.00 | 160.00 | 8 |
| ATOM | 21079 | N | GLU | H | 98 | 65.756 | 44.101 | 41.444 | 1.00 | 110.78 | 7 |
| ATOM | 21080 | CA | GLU | H | 98 | 65.862 | 44.776 | 40.150 | 1.00 | 110.73 | 6 |
| ATOM | 21081 | CB | GLU | H | 98 | 67.267 | 45.354 | 39.958 | 1.00 | 111.96 | 6 |
| ATOM | 21082 | CG | GLU | H | 98 | 67.793 | 46.113 | 41.160 | 1.00 | 112.01 | 6 |
| ATOM | 21083 | CD | GLU | H | 98 | 69.141 | 46.736 | 40.902 | 1.00 | 112.19 | 6 |
| ATOM | 21084 | OE1 | GLU | H | 98 | 69.977 | 46.079 | 40.248 | 1.00 | 111.88 | 8 |
| ATOM | 21085 | OE2 | GLU | H | 98 | 69.369 | 47.874 | 41.363 | 1.00 | 113.13 | 8 |
| ATOM | 21086 | C | GLU | H | 98 | 65.553 | 43.817 | 38.999 | 1.00 | 110.66 | 6 |
| ATOM | 21087 | O | GLU | H | 98 | 65.243 | 42.639 | 39.275 | 1.00 | 110.53 | 8 |
| ATOM | 21088 | OXT | GLU | H | 98 | 65.625 | 44.257 | 37.832 | 1.00 | 112.67 | 8 |
| ATOM | 21089 | MG + 2 | MG2 | I | 1 | 45.303 | 87.748 | 42.601 | 1.00 | 145.66 | 12 |
| ATOM | 21090 | ZN + 2 | ZN2 | J | 1 | 79.626 | 86.200 | 67.933 | 1.00 | 79.71 | 30 |
| ATOM | 21091 | CB | ALA | K | 2 | 151.234 | 102.312 | −39.510 | 1.00 | 22.98 | 6 |
| ATOM | 21092 | C | ALA | K | 2 | 151.505 | 104.365 | −40.898 | 1.00 | 83.38 | 6 |
| ATOM | 21093 | O | ALA | K | 2 | 151.175 | 103.658 | −41.854 | 1.00 | 84.27 | 8 |
| ATOM | 21094 | N | ALA | K | 2 | 150.387 | 104.457 | −38.685 | 1.00 | 82.24 | 7 |
| ATOM | 21095 | CA | ALA | K | 2 | 151.466 | 103.808 | −39.477 | 1.00 | 82.79 | 6 |
| ATOM | 21096 | N | ALA | K | 3 | 151.919 | 105.625 | −41.035 | 1.00 | 51.82 | 7 |
| ATOM | 21097 | CA | ALA | K | 3 | 151.976 | 106.267 | −42.346 | 1.00 | 51.87 | 6 |
| ATOM | 21098 | CB | ALA | K | 3 | 150.787 | 107.196 | −42.508 | 1.00 | 101.03 | 6 |
| ATOM | 21099 | C | ALA | K | 3 | 153.262 | 107.042 | −42.585 | 1.00 | 51.85 | 6 |
| ATOM | 21100 | O | ALA | K | 3 | 154.113 | 107.138 | −41.711 | 1.00 | 51.08 | 8 |
| ATOM | 21101 | N | ALA | K | 4 | 153.386 | 107.602 | −43.783 | 1.00 | 125.85 | 7 |
| ATOM | 21102 | CA | ALA | K | 4 | 154.552 | 108.394 | −44.163 | 1.00 | 126.96 | 6 |
| ATOM | 21103 | CB | ALA | K | 4 | 155.346 | 107.661 | −45.247 | 1.00 | 55.29 | 6 |
| ATOM | 21104 | C | ALA | K | 4 | 154.047 | 109.740 | −44.687 | 1.00 | 126.86 | 6 |
| ATOM | 21105 | O | ALA | K | 4 | 152.916 | 109.822 | −45.161 | 1.00 | 127.60 | 8 |
| ATOM | 21106 | N | ALA | K | 5 | 154.857 | 110.794 | −44.597 | 1.00 | 32.02 | 7 |
| ATOM | 21107 | CA | ALA | K | 5 | 154.416 | 112.103 | −45.090 | 1.00 | 33.06 | 6 |
| ATOM | 21108 | CB | ALA | K | 5 | 153.189 | 112.582 | −44.302 | 1.00 | 19.54 | 6 |
| ATOM | 21109 | C | ALA | K | 5 | 155.498 | 113.172 | −45.040 | 1.00 | 34.91 | 6 |
| ATOM | 21110 | O | ALA | K | 5 | 155.279 | 114.250 | −44.471 | 1.00 | 34.68 | 8 |
| ATOM | 21111 | N | ALA | K | 6 | 156.642 | 112.870 | −45.656 | 1.00 | 98.60 | 7 |
| ATOM | 21112 | CA | ALA | K | 6 | 157.816 | 113.753 | −45.709 | 1.00 | 100.93 | 6 |
| ATOM | 21113 | CB | ALA | K | 6 | 158.805 | 113.233 | −46.750 | 1.00 | 59.34 | 6 |
| ATOM | 21114 | C | ALA | K | 6 | 157.551 | 115.238 | −45.963 | 1.00 | 102.61 | 6 |
| ATOM | 21115 | O | ALA | K | 6 | 156.952 | 115.616 | −46.968 | 1.00 | 103.52 | 8 |
| ATOM | 21116 | N | ALA | K | 7 | 158.027 | 116.073 | −45.044 | 1.00 | 67.32 | 7 |
| ATOM | 21117 | CA | ALA | K | 7 | 157.875 | 117.517 | −45.135 | 1.00 | 68.87 | 6 |
| ATOM | 21118 | CB | ALA | K | 7 | 157.396 | 118.086 | −43.804 | 1.00 | 59.22 | 6 |
| ATOM | 21119 | C | ALA | K | 7 | 159.251 | 118.059 | −45.479 | 1.00 | 70.43 | 6 |
| ATOM | 21120 | O | ALA | K | 7 | 160.210 | 117.286 | −45.581 | 1.00 | 70.10 | 8 |
| ATOM | 21121 | N | ALA | K | 8 | 159.345 | 119.381 | −45.641 | 1.00 | 96.74 | 7 |
| ATOM | 21122 | CA | ALA | K | 8 | 160.602 | 120.031 | −46.006 | 1.00 | 98.15 | 6 |
| ATOM | 21123 | CB | ALA | K | 8 | 160.624 | 120.282 | −47.503 | 1.00 | 53.20 | 6 |
| ATOM | 21124 | C | ALA | K | 8 | 160.909 | 121.336 | −45.278 | 1.00 | 99.80 | 6 |
| ATOM | 21125 | O | ALA | K | 8 | 161.739 | 121.374 | −44.366 | 1.00 | 100.81 | 8 |
| ATOM | 21126 | N | ALA | K | 9 | 160.242 | 122.404 | −45.705 | 1.00 | 208.87 | 7 |
| ATOM | 21127 | CA | ALA | K | 9 | 160.441 | 123.740 | −45.149 | 1.00 | 208.87 | 6 |
| ATOM | 21128 | CB | ALA | K | 9 | 159.677 | 124.758 | −45.998 | 1.00 | 125.45 | 6 |
| ATOM | 21129 | C | ALA | K | 9 | 160.070 | 123.913 | −43.675 | 1.00 | 208.87 | 6 |
| ATOM | 21130 | O | ALA | K | 9 | 159.044 | 124.520 | −43.363 | 1.00 | 208.87 | 8 |
| ATOM | 21131 | N | ALA | K | 10 | 160.917 | 123.396 | −42.785 | 1.00 | 93.41 | 7 |
| ATOM | 21132 | CA | ALA | K | 10 | 160.723 | 123.483 | −41.337 | 1.00 | 93.15 | 6 |
| ATOM | 21133 | CB | ALA | K | 10 | 159.242 | 123.417 | −40.976 | 1.00 | 102.75 | 6 |
| ATOM | 21134 | C | ALA | K | 10 | 161.457 | 122.343 | −40.657 | 1.00 | 93.40 | 6 |
| ATOM | 21135 | O | ALA | K | 10 | 162.666 | 122.518 | −40.399 | 1.00 | 93.15 | 8 |
| ATOM | 21136 | OXT | ALA | K | 10 | 160.818 | 121.292 | −40.408 | 1.00 | 101.68 | 8 |
| ATOM | 21137 | CB | ALA | L | 20 | 150.457 | 116.704 | −34.486 | 1.00 | 66.36 | 6 |
| ATOM | 21138 | C | ALA | L | 20 | 148.118 | 115.853 | −34.285 | 1.00 | 151.44 | 6 |
| ATOM | 21139 | O | ALA | L | 20 | 148.618 | 114.738 | −34.438 | 1.00 | 151.99 | 8 |
| ATOM | 21140 | N | ALA | L | 20 | 148.904 | 117.702 | −32.833 | 1.00 | 151.58 | 7 |
| ATOM | 21141 | CA | ALA | L | 20 | 149.006 | 117.084 | −34.186 | 1.00 | 151.20 | 6 |
| ATOM | 21142 | N | ALA | L | 21 | 146.808 | 116.070 | −34.187 | 1.00 | 57.67 | 7 |
| ATOM | 21143 | CA | ALA | L | 21 | 145.801 | 115.009 | −34.274 | 1.00 | 57.13 | 6 |
| ATOM | 21144 | CB | ALA | L | 21 | 146.357 | 113.798 | −35.032 | 1.00 | 33.92 | 6 |
| ATOM | 21145 | C | ALA | L | 21 | 145.269 | 114.562 | −32.922 | 1.00 | 57.24 | 6 |
| ATOM | 21146 | O | ALA | L | 21 | 144.757 | 115.363 | −32.147 | 1.00 | 56.89 | 8 |
| ATOM | 21147 | N | ALA | L | 22 | 145.388 | 113.265 | −32.662 | 1.00 | 126.91 | 7 |
| ATOM | 21148 | CA | ALA | L | 22 | 144.933 | 112.655 | −31.421 | 1.00 | 128.43 | 6 |
| ATOM | 21149 | CB | ALA | L | 22 | 143.412 | 112.529 | −31.423 | 1.00 | 95.96 | 6 |
| ATOM | 21150 | C | ALA | L | 22 | 145.576 | 111.280 | −31.379 | 1.00 | 129.17 | 6 |
| ATOM | 21151 | O | ALA | L | 22 | 144.910 | 110.274 | −31.137 | 1.00 | 130.00 | 8 |
| ATOM | 21152 | N | ALA | L | 23 | 146.882 | 111.247 | −31.626 | 1.00 | 103.66 | 7 |
| ATOM | 21153 | CA | ALA | L | 23 | 147.622 | 109.996 | −31.644 | 1.00 | 102.70 | 6 |
| ATOM | 21154 | CB | ALA | L | 23 | 147.159 | 109.169 | −32.809 | 1.00 | 57.04 | 6 |
| ATOM | 21155 | C | ALA | L | 23 | 149.117 | 110.255 | −31.758 | 1.00 | 102.46 | 6 |
| ATOM | 21156 | O | ALA | L | 23 | 149.556 | 110.931 | −32.688 | 1.00 | 102.85 | 8 |
| ATOM | 21157 | N | ALA | L | 24 | 149.891 | 109.702 | −30.822 | 1.00 | 74.77 | 7 |
| ATOM | 21158 | CA | ALA | L | 24 | 151.350 | 109.879 | −30.802 | 1.00 | 74.64 | 6 |
| ATOM | 21159 | CB | ALA | L | 24 | 151.956 | 109.157 | −29.580 | 1.00 | 17.69 | 6 |
| ATOM | 21160 | C | ALA | L | 24 | 152.031 | 109.394 | −32.081 | 1.00 | 74.88 | 6 |
| ATOM | 21161 | O | ALA | L | 24 | 151.611 | 109.746 | −33.184 | 1.00 | 74.18 | 8 |
| ATOM | 21162 | N | ALA | L | 25 | 153.093 | 108.605 | −31.911 | 1.00 | 94.81 | 7 |
| ATOM | 21163 | CA | ALA | L | 25 | 153.848 | 108.053 | −33.031 | 1.00 | 96.36 | 6 |
| ATOM | 21164 | CB | ALA | L | 25 | 153.794 | 109.014 | −34.212 | 1.00 | 54.01 | 6 |
| ATOM | 21165 | C | ALA | L | 25 | 155.314 | 107.732 | −32.718 | 1.00 | 97.37 | 6 |
| ATOM | 21166 | O | ALA | L | 25 | 155.667 | 107.220 | −31.648 | 1.00 | 98.51 | 8 |
| ATOM | 21167 | N | ALA | L | 26 | 156.147 | 108.037 | −33.707 | 1.00 | 88.34 | 7 |
| ATOM | 21168 | CA | ALA | L | 26 | 157.591 | 107.854 | −33.677 | 1.00 | 87.95 | 6 |
| ATOM | 21169 | CB | ALA | L | 26 | 157.943 | 106.396 | −33.726 | 1.00 | 13.87 | 6 |
| ATOM | 21170 | C | ALA | L | 26 | 158.024 | 108.552 | −34.956 | 1.00 | 88.24 | 6 |
| ATOM | 21171 | O | ALA | L | 26 | 157.241 | 109.298 | −35.542 | 1.00 | 88.35 | 8 |
| ATOM | 21172 | N | ALA | L | 27 | 159.246 | 108.323 | −35.410 | 1.00 | 76.54 | 7 |
| ATOM | 21173 | CA | ALA | L | 27 | 159.689 | 108.997 | −36.621 | 1.00 | 77.54 | 6 |
| ATOM | 21174 | CB | ALA | L | 27 | 160.137 | 110.415 | −36.290 | 1.00 | 85.17 | 6 |
| ATOM | 21175 | C | ALA | L | 27 | 160.807 | 108.250 | −37.306 | 1.00 | 78.24 | 6 |
| ATOM | 21176 | O | ALA | L | 27 | 161.356 | 107.303 | −36.752 | 1.00 | 78.60 | 8 |
| ATOM | 21177 | N | ALA | L | 28 | 161.141 | 108.681 | −38.516 | 1.00 | 55.91 | 7 |
| ATOM | 21178 | CA | ALA | L | 28 | 162.207 | 108.050 | −39.273 | 1.00 | 56.77 | 6 |
| ATOM | 21179 | CB | ALA | L | 28 | 161.822 | 106.625 | −39.624 | 1.00 | 46.12 | 6 |
| ATOM | 21180 | C | ALA | L | 28 | 162.499 | 108.841 | −40.538 | 1.00 | 57.75 | 6 |
| ATOM | 21181 | O | ALA | L | 28 | 161.829 | 109.828 | −40.823 | 1.00 | 58.27 | 8 |
| ATOM | 21182 | N | ALA | L | 29 | 163.516 | 108.405 | −41.279 | 1.00 | 96.75 | 7 |
| ATOM | 21183 | CA | ALA | L | 29 | 163.925 | 109.039 | −42.533 | 1.00 | 98.03 | 6 |
| ATOM | 21184 | CB | ALA | L | 29 | 162.711 | 109.221 | −43.441 | 1.00 | 147.96 | 6 |
| ATOM | 21185 | C | ALA | L | 29 | 164.669 | 110.366 | −42.371 | 1.00 | 98.81 | 6 |
| ATOM | 21186 | O | ALA | L | 29 | 164.579 | 111.028 | −41.337 | 1.00 | 98.47 | 8 |
| ATOM | 21187 | N | ALA | L | 30 | 165.399 | 110.745 | −43.416 | 1.00 | 103.95 | 7 |
| ATOM | 21188 | CA | ALA | L | 30 | 166.180 | 111.973 | −43.411 | 1.00 | 105.83 | 6 |
| ATOM | 21189 | CB | ALA | L | 30 | 167.160 | 111.939 | −42.251 | 1.00 | 117.60 | 6 |
| ATOM | 21190 | C | ALA | L | 30 | 166.941 | 112.148 | −44.727 | 1.00 | 106.99 | 6 |
| ATOM | 21191 | O | ALA | L | 30 | 167.149 | 111.178 | −45.452 | 1.00 | 107.48 | 8 |
| ATOM | 21192 | N | ALA | L | 31 | 167.350 | 113.388 | −45.014 | 1.00 | 111.67 | 7 |
| ATOM | 21193 | CA | ALA | L | 31 | 168.109 | 113.767 | −46.217 | 1.00 | 111.87 | 6 |
| ATOM | 21194 | CB | ALA | L | 31 | 168.839 | 112.555 | −46.811 | 1.00 | 111.02 | 6 |
| ATOM | 21195 | C | ALA | L | 31 | 167.275 | 114.446 | −47.299 | 1.00 | 112.34 | 6 |
| ATOM | 21196 | O | ALA | L | 31 | 166.042 | 114.409 | −47.266 | 1.00 | 112.64 | 8 |
| ATOM | 21197 | N | ALA | L | 32 | 167.977 | 115.061 | −48.253 | 1.00 | 104.58 | 7 |
| ATOM | 21198 | CA | ALA | L | 32 | 167.382 | 115.779 | −49.382 | 1.00 | 105.61 | 6 |
| ATOM | 21199 | CB | ALA | L | 32 | 166.051 | 115.139 | −49.788 | 1.00 | 131.34 | 6 |
| ATOM | 21200 | C | ALA | L | 32 | 167.174 | 117.252 | −49.052 | 1.00 | 106.17 | 6 |
| ATOM | 21201 | O | ALA | L | 32 | 167.865 | 117.811 | −48.201 | 1.00 | 106.73 | 8 |
| ATOM | 21202 | N | ALA | L | 33 | 166.230 | 117.879 | −49.746 | 1.00 | 164.25 | 7 |
| ATOM | 21203 | CA | ALA | L | 33 | 165.906 | 119.281 | −49.511 | 1.00 | 164.22 | 6 |
| ATOM | 21204 | CB | ALA | L | 33 | 165.548 | 119.970 | −50.816 | 1.00 | 122.86 | 6 |
| ATOM | 21205 | C | ALA | L | 33 | 164.712 | 119.288 | −48.572 | 1.00 | 164.08 | 6 |
| ATOM | 21206 | O | ALA | L | 33 | 164.339 | 120.325 | −48.023 | 1.00 | 164.22 | 8 |
| ATOM | 21207 | N | ALA | L | 34 | 164.120 | 118.108 | −48.407 | 1.00 | 94.79 | 7 |
| ATOM | 21208 | CA | ALA | L | 34 | 162.967 | 117.918 | −47.538 | 1.00 | 94.68 | 6 |
| ATOM | 21209 | CB | ALA | L | 34 | 161.853 | 117.207 | −48.293 | 1.00 | 157.98 | 6 |
| ATOM | 21210 | C | ALA | L | 34 | 163.375 | 117.106 | −46.316 | 1.00 | 94.23 | 6 |
| ATOM | 21211 | O | ALA | L | 34 | 164.406 | 116.437 | −46.320 | 1.00 | 94.34 | 8 |
| ATOM | 21212 | N | ALA | L | 35 | 162.555 | 117.156 | −45.275 | 1.00 | 121.08 | 7 |
| ATOM | 21213 | CA | ALA | L | 35 | 162.844 | 116.447 | −44.040 | 1.00 | 120.47 | 6 |
| ATOM | 21214 | CB | ALA | L | 35 | 161.951 | 116.988 | −42.928 | 1.00 | 141.46 | 6 |
| ATOM | 21215 | C | ALA | L | 35 | 162.714 | 114.925 | −44.114 | 1.00 | 119.62 | 6 |
| ATOM | 21216 | O | ALA | L | 35 | 163.622 | 114.200 | −43.703 | 1.00 | 120.56 | 8 |
| ATOM | 21217 | N | ALA | L | 36 | 161.588 | 114.457 | −44.646 | 1.00 | 149.03 | 7 |
| ATOM | 21218 | CA | ALA | L | 36 | 161.273 | 113.030 | −44.744 | 1.00 | 146.37 | 6 |
| ATOM | 21219 | CB | ALA | L | 36 | 162.551 | 112.168 | −44.670 | 1.00 | 13.87 | 6 |
| ATOM | 21220 | C | ALA | L | 36 | 160.370 | 112.780 | −43.534 | 1.00 | 145.31 | 6 |
| ATOM | 21221 | O | ALA | L | 36 | 160.173 | 113.687 | −42.726 | 1.00 | 146.01 | 8 |
| ATOM | 21222 | N | ALA | L | 37 | 159.811 | 111.586 | −43.385 | 1.00 | 120.00 | 7 |
| ATOM | 21223 | CA | ALA | L | 37 | 158.935 | 111.367 | −42.240 | 1.00 | 118.54 | 6 |
| ATOM | 21224 | CB | ALA | L | 37 | 157.533 | 111.800 | −42.593 | 1.00 | 13.87 | 6 |
| ATOM | 21225 | C | ALA | L | 37 | 158.906 | 109.951 | −41.685 | 1.00 | 117.96 | 6 |
| ATOM | 21226 | O | ALA | L | 37 | 159.343 | 109.710 | −40.561 | 1.00 | 118.82 | 8 |
| ATOM | 21227 | N | ALA | L | 38 | 158.379 | 109.022 | −42.476 | 1.00 | 54.35 | 7 |
| ATOM | 21228 | CA | ALA | L | 38 | 158.258 | 107.624 | −42.071 | 1.00 | 52.50 | 6 |
| ATOM | 21229 | CB | ALA | L | 38 | 159.641 | 106.994 | −41.961 | 1.00 | 83.35 | 6 |
| ATOM | 21230 | C | ALA | L | 38 | 157.537 | 107.601 | −40.725 | 1.00 | 51.68 | 6 |
| ATOM | 21231 | O | ALA | L | 38 | 157.867 | 106.829 | −39.827 | 1.00 | 51.03 | 8 |
| ATOM | 21232 | N | ALA | L | 39 | 156.546 | 108.476 | −40.605 | 1.00 | 111.17 | 7 |
| ATOM | 21233 | CA | ALA | L | 39 | 155.754 | 108.608 | −39.392 | 1.00 | 110.47 | 6 |
| ATOM | 21234 | CB | ALA | L | 39 | 154.718 | 109.724 | −39.580 | 1.00 | 13.87 | 6 |
| ATOM | 21235 | C | ALA | L | 39 | 155.073 | 107.288 | −39.010 | 1.00 | 110.18 | 6 |
| ATOM | 21236 | O | ALA | L | 39 | 154.927 | 106.394 | −39.838 | 1.00 | 110.89 | 8 |
| ATOM | 21237 | N | ALA | L | 40 | 154.680 | 107.165 | −37.745 | 1.00 | 120.20 | 7 |
| ATOM | 21238 | CA | ALA | L | 40 | 154.021 | 105.958 | −37.249 | 1.00 | 119.91 | 6 |
| ATOM | 21239 | CB | ALA | L | 40 | 155.037 | 105.079 | −36.496 | 1.00 | 46.74 | 6 |
| ATOM | 21240 | C | ALA | L | 40 | 152.865 | 106.354 | −36.327 | 1.00 | 119.74 | 6 |
| ATOM | 21241 | O | ALA | L | 40 | 152.980 | 106.272 | −35.103 | 1.00 | 119.60 | 8 |
| ATOM | 21242 | N | ALA | L | 41 | 151.757 | 106.776 | −36.941 | 1.00 | 133.41 | 7 |
| ATOM | 21243 | CA | ALA | L | 41 | 150.545 | 107.232 | −36.246 | 1.00 | 132.82 | 6 |
| ATOM | 21244 | CB | ALA | L | 41 | 149.384 | 107.316 | −37.244 | 1.00 | 66.73 | 6 |
| ATOM | 21245 | C | ALA | L | 41 | 150.109 | 106.421 | −35.026 | 1.00 | 132.26 | 6 |
| ATOM | 21246 | O | ALA | L | 41 | 150.834 | 105.544 | −34.558 | 1.00 | 132.13 | 8 |
| ATOM | 21247 | N | ALA | L | 42 | 148.918 | 106.736 | −34.513 | 1.00 | 46.03 | 7 |
| ATOM | 21248 | CA | ALA | L | 42 | 148.350 | 106.041 | −33.351 | 1.00 | 45.89 | 6 |
| ATOM | 21249 | CB | ALA | L | 42 | 149.065 | 106.489 | −32.056 | 1.00 | 22.55 | 6 |
| ATOM | 21250 | C | ALA | L | 42 | 146.832 | 106.230 | −33.212 | 1.00 | 45.75 | 6 |
| ATOM | 21251 | O | ALA | L | 42 | 146.245 | 105.736 | −32.258 | 1.00 | 45.13 | 8 |
| ATOM | 21252 | N | ALA | L | 43 | 146.226 | 106.930 | −34.178 | 1.00 | 73.48 | 7 |
| ATOM | 21253 | CA | ALA | L | 43 | 144.787 | 107.246 | −34.247 | 1.00 | 74.48 | 6 |
| ATOM | 21254 | CB | ALA | L | 43 | 144.183 | 106.632 | −35.526 | 1.00 | 13.87 | 6 |
| ATOM | 21255 | C | ALA | L | 43 | 143.926 | 106.888 | −33.023 | 1.00 | 75.95 | 6 |
| ATOM | 21256 | O | ALA | L | 43 | 144.440 | 106.783 | −31.910 | 1.00 | 76.87 | 8 |
| ATOM | 21257 | N | ALA | L | 44 | 142.613 | 106.744 | −33.234 | 1.00 | 68.37 | 7 |
| ATOM | 21258 | CA | ALA | L | 44 | 141.635 | 106.403 | −32.177 | 1.00 | 68.58 | 6 |
| ATOM | 21259 | CB | ALA | L | 44 | 142.028 | 107.039 | −30.815 | 1.00 | 20.12 | 6 |
| ATOM | 21260 | C | ALA | L | 44 | 140.237 | 106.883 | −32.575 | 1.00 | 69.21 | 6 |
| ATOM | 21261 | O | ALA | L | 44 | 139.956 | 108.086 | −32.558 | 1.00 | 68.33 | 8 |
| ATOM | 21262 | N | ALA | L | 45 | 139.362 | 105.944 | −32.921 | 1.00 | 150.12 | 7 |
| ATOM | 21263 | CA | ALA | L | 45 | 138.005 | 106.290 | −33.332 | 1.00 | 151.81 | 6 |
| ATOM | 21264 | CB | ALA | L | 45 | 137.756 | 105.826 | −34.766 | 1.00 | 123.14 | 6 |
| ATOM | 21265 | C | ALA | L | 45 | 136.932 | 105.726 | −32.410 | 1.00 | 152.61 | 6 |
| ATOM | 21266 | O | ALA | L | 45 | 137.153 | 105.562 | −31.212 | 1.00 | 153.03 | 8 |
| ATOM | 21267 | N | ALA | L | 46 | 135.767 | 105.437 | −32.986 | 1.00 | 98.73 | 7 |
| ATOM | 21268 | CA | ALA | L | 46 | 134.631 | 104.908 | −32.240 | 1.00 | 99.54 | 6 |
| ATOM | 21269 | CB | ALA | L | 46 | 133.534 | 104.474 | −33.203 | 1.00 | 171.10 | 6 |
| ATOM | 21270 | C | ALA | L | 46 | 135.021 | 103.745 | −31.343 | 1.00 | 100.29 | 6 |
| ATOM | 21271 | O | ALA | L | 46 | 134.824 | 103.871 | −30.115 | 1.00 | 100.99 | 8 |
| ATOM | 21272 | OXT | ALA | L | 46 | 135.514 | 102.728 | −31.875 | 1.00 | 172.19 | 8 |
| ATOM | 21273 | C1 | RIF | R | 1 | 48.535 | 97.571 | 24.909 | 1.00 | 48.44 | 6 |
| ATOM | 21274 | C2 | RIF | R | 1 | 50.953 | 96.931 | 24.770 | 1.00 | 48.44 | 6 |
| ATOM | 21275 | C3 | RIF | R | 1 | 53.514 | 96.552 | 24.129 | 1.00 | 48.44 | 6 |
| ATOM | 21276 | C4 | RIF | R | 1 | 54.396 | 97.190 | 23.105 | 1.00 | 48.44 | 6 |
| ATOM | 21277 | C5 | RIF | R | 1 | 55.661 | 97.778 | 23.665 | 1.00 | 48.44 | 6 |
| ATOM | 21278 | C6 | RIF | R | 1 | 51.493 | 100.052 | 21.561 | 1.00 | 48.44 | 6 |
| ATOM | 21279 | C7 | RIF | R | 1 | 46.186 | 97.136 | 26.970 | 1.00 | 48.44 | 6 |
| ATOM | 21280 | C8 | RIF | R | 1 | 44.950 | 96.432 | 27.462 | 1.00 | 48.44 | 6 |
| ATOM | 21281 | C9 | RIF | R | 1 | 44.290 | 95.430 | 26.775 | 1.00 | 48.44 | 6 |
| ATOM | 21282 | C10 | RIF | R | 1 | 44.591 | 94.823 | 25.453 | 1.00 | 48.44 | 6 |
| ATOM | 21283 | C11 | RIF | R | 1 | 43.975 | 93.708 | 25.000 | 1.00 | 48.44 | 6 |
| ATOM | 21284 | C12 | RIF | R | 1 | 48.385 | 96.576 | 25.931 | 1.00 | 48.44 | 6 |
| ATOM | 21285 | C13 | RIF | R | 1 | 44.608 | 92.418 | 24.492 | 1.00 | 48.44 | 6 |
| ATOM | 21286 | C14 | RIF | R | 1 | 45.584 | 92.657 | 23.331 | 1.00 | 48.44 | 6 |
| ATOM | 21287 | C15 | RIF | R | 1 | 46.563 | 91.517 | 22.975 | 1.00 | 48.44 | 6 |
| ATOM | 21288 | C16 | RIF | R | 1 | 47.602 | 91.868 | 21.900 | 1.00 | 48.44 | 6 |
| ATOM | 21289 | C17 | RIF | R | 1 | 49.044 | 92.222 | 22.338 | 1.00 | 48.44 | 6 |
| ATOM | 21290 | C18 | RIF | R | 1 | 49.802 | 92.855 | 21.145 | 1.00 | 48.44 | 6 |
| ATOM | 21291 | C19 | RIF | R | 1 | 50.807 | 93.988 | 21.507 | 1.00 | 48.44 | 6 |
| ATOM | 21292 | C20 | RIF | R | 1 | 51.788 | 94.290 | 20.347 | 1.00 | 48.44 | 6 |
| ATOM | 21293 | C21 | RIF | R | 1 | 52.908 | 95.313 | 20.658 | 1.00 | 48.44 | 6 |
| ATOM | 21294 | C22 | RIF | R | 1 | 53.811 | 95.149 | 21.637 | 1.00 | 48.44 | 6 |
| ATOM | 21295 | C23 | RIF | R | 1 | 49.457 | 95.732 | 26.315 | 1.00 | 48.44 | 6 |
| ATOM | 21296 | C24 | RIF | R | 1 | 44.502 | 96.928 | 28.818 | 1.00 | 48.44 | 6 |
| ATOM | 21297 | C25 | RIF | R | 1 | 49.289 | 94.561 | 27.183 | 1.00 | 48.44 | 6 |
| ATOM | 21298 | C26 | RIF | R | 1 | 46.669 | 93.510 | 29.331 | 1.00 | 48.44 | 6 |
| ATOM | 21299 | C27 | RIF | R | 1 | 46.063 | 92.158 | 29.654 | 1.00 | 48.44 | 6 |
| ATOM | 21300 | C28 | RIF | R | 1 | 48.361 | 91.198 | 29.811 | 1.00 | 48.44 | 6 |
| ATOM | 21301 | C29 | RIF | R | 1 | 48.909 | 92.576 | 29.465 | 1.00 | 48.44 | 6 |
| ATOM | 21302 | C30 | RIF | R | 1 | 46.437 | 89.991 | 30.793 | 1.00 | 48.44 | 6 |
| ATOM | 21303 | C31 | RIF | R | 1 | 45.276 | 91.766 | 25.727 | 1.00 | 48.44 | 6 |
| ATOM | 21304 | C32 | RIF | R | 1 | 45.824 | 90.223 | 22.532 | 1.00 | 48.44 | 6 |
| ATOM | 21305 | C33 | RIF | R | 1 | 49.817 | 91.008 | 22.892 | 1.00 | 48.44 | 6 |
| ATOM | 21306 | C34 | RIF | R | 1 | 49.995 | 95.193 | 21.899 | 1.00 | 48.44 | 6 |
| ATOM | 21307 | C35 | RIF | R | 1 | 49.896 | 91.280 | 19.314 | 1.00 | 48.44 | 6 |
| ATOM | 21308 | C36 | RIF | R | 1 | 50.711 | 90.240 | 18.695 | 1.00 | 48.44 | 6 |
| ATOM | 21309 | C37 | RIF | R | 1 | 51.509 | 94.202 | 17.934 | 1.00 | 48.44 | 6 |
| ATOM | 21310 | C38 | RIF | R | 1 | 50.740 | 95.963 | 25.789 | 1.00 | 48.44 | 6 |
| ATOM | 21311 | C39 | RIF | R | 1 | 52.255 | 97.183 | 24.089 | 1.00 | 48.44 | 6 |
| ATOM | 21312 | C40 | RIF | R | 1 | 52.329 | 98.184 | 23.066 | 1.00 | 48.44 | 6 |
| ATOM | 21313 | C41 | RIF | R | 1 | 51.324 | 99.013 | 22.634 | 1.00 | 48.44 | 6 |
| ATOM | 21314 | C42 | RIF | R | 1 | 50.010 | 98.822 | 23.251 | 1.00 | 48.44 | 6 |
| ATOM | 21315 | C43 | RIF | R | 1 | 49.837 | 97.769 | 24.323 | 1.00 | 48.44 | 6 |
| ATOM | 21316 | N1 | RIF | R | 1 | 48.294 | 94.494 | 28.016 | 1.00 | 48.44 | 7 |
| ATOM | 21317 | N2 | RIF | R | 1 | 47.948 | 93.290 | 28.636 | 1.00 | 48.44 | 7 |
| ATOM | 21318 | N3 | RIF | R | 1 | 47.003 | 91.327 | 30.489 | 1.00 | 48.44 | 7 |
| ATOM | 21319 | N4 | RIF | R | 1 | 47.096 | 96.286 | 26.473 | 1.00 | 48.44 | 7 |
| ATOM | 21320 | O1 | RIF | R | 1 | 47.471 | 98.266 | 24.488 | 1.00 | 48.44 | 8 |
| ATOM | 21321 | O2 | RIF | R | 1 | 49.006 | 99.538 | 22.893 | 1.00 | 48.44 | 8 |
| ATOM | 21322 | O3 | RIF | R | 1 | 53.573 | 98.238 | 22.501 | 1.00 | 48.44 | 8 |
| ATOM | 21323 | O4 | RIF | R | 1 | 53.974 | 95.598 | 24.821 | 1.00 | 48.44 | 8 |
| ATOM | 21324 | O5 | RIF | R | 1 | 54.773 | 96.192 | 22.056 | 1.00 | 48.44 | 8 |
| ATOM | 21325 | O6 | RIF | R | 1 | 50.986 | 94.738 | 19.186 | 1.00 | 48.44 | 8 |
| ATOM | 21326 | O7 | RIF | R | 1 | 50.469 | 91.795 | 20.428 | 1.00 | 48.44 | 8 |
| ATOM | 21327 | O8 | RIF | R | 1 | 48.856 | 91.664 | 18.913 | 1.00 | 48.44 | 8 |
| ATOM | 21328 | O9 | RIF | R | 1 | 47.161 | 92.980 | 21.083 | 1.00 | 48.44 | 8 |
| ATOM | 21329 | O10 | RIF | R | 1 | 44.756 | 92.982 | 22.212 | 1.00 | 48.44 | 8 |
| ATOM | 21330 | O16 | RIF | R | 1 | 46.290 | 98.343 | 27.047 | 1.00 | 48.44 | 8 |
| ATOM | 21331 | O17 | RIF | R | 1 | 51.732 | 95.163 | 26.318 | 1.00 | 48.44 | 8 |
| END | | | | | | | | | | | |

What is claimed is:

1. A crystal of rifampicin bound to a core RNA polymerase (Rif-RNAP) that effectively diffracts X-rays for the determination of the atomic coordinates to a resolution of better than 3.5 Angstroms.

2. The crystal of claim 1, wherein the core RNA polymerase is a bacterial core RNA polymerase.

3. The crystal of claim 2, wherein the bacterial core RNA polymerase is a thermophilic bacterial core RNA polymerase.

4. The crystal of claim 3, wherein the thermophilic bacterial core RNA polymerase is a Thermus aquaticus bacterial core RNA polymerase.

5. The crystal of claim 1, wherein the core RNA polymerase comprises a β′ subunit, a β subunit, and a pair of α subunits.

6. The crystal of claim 5, further comprising an ω subunit.

7. The crystal of claim 1 that effectively diffracts X-rays for the determination of the atomic coordinates of the core RNA polymerase to a resolution of 3.3 Angstroms or better.

8. The crystal of claim 7 having space group of P4₁2₁2 and a unit cell of dimensions of a=b=201 and c=294 Å.

9. A method of identifying an agent for use as an inhibitor of bacterial RNA polymerase comprising:
(a) obtaining a set of atomic coordinates defining the three-dimensional structure of rifampicin bound to the core RNA polymerase (Rif-RNAP); wherein said core RNAP consists essentially of the β′, β, α and ω subunits of RNAP from T. aquaticus and using a crystal having the space group of P4₁2₁2 and unit cell dimensions of a=b=201 and c=294 Å;
(b) selecting a potential agent by performing rational drug design with the atomic coordinates obtained in step (a), wherein said selecting is performed in conjunction with computer modeling;
(c) contacting the potential agent with a bacterial RNA polymerase; and
(d) measuring the activity of the bacterial RNA polymerase; wherein a potential agent is identified as an agent that inhibits bacterial RNA polymerase when there is a decrease in the activity of the bacterial RNA polymerase in the presence of the agent relative to in its absence.

10. The method of claim 9, further comprising:
(e) preparing a supplemental crystal containing the core RNA polymerase formed in the presence of the potential agent, wherein the crystal effectively diffracts X-rays for the determination of the atomic coordinates to a resolution of better than 5.0 Angstroms;
(f) determining the three-dimensional coordinates of the supplemental crystal with molecular replacement analysis; and
(g) selecting a second generation agent by performing rational drug design with the three-dimensional coordinates determined for the supplemental crystal, wherein said selecting is performed in conjunction with computer modeling.

11. The method of claim 10, further comprising:
(h) contacting the second generation agent with a eukaryotic RNA polymerase; and
(i) measuring the activity of the eukaryotic RNA polymerase; wherein an agent is identified as an agent for use as an inhibitor of bacterial RNA polymerase when there is no change in the activity of the eukaryotic RNA polymerase in the presence of the agent relative to in its absence; and wherein the agent identified inhibits bacterial but not eukaryotic RNA polymerase.

12. A method of identifying an agent that inhibits bacterial growth comprising:
(a) obtaining a set of atomic coordinates defining the three-dimensional structure of rifampicin bound to core RNA polymerase (Rif-RNAP); wherein the core RNA polymerase consists essentially of the β′, β, α and ω subunits of RNAP from T. aquaticus and using a crystal having the space group of P4₁2₁2 and unit cell dimensions of a=b=201 and c=294 Å;
(b) selecting a potential agent by performing rational drug design with the atomic coordinates obtained in step (a), wherein said selecting is performed in conjunction with computer modeling;
(c) contacting the potential agent with a bacterial culture; and
(d) measuring the growth of the bacterial culture under conditions in which the bacterial culture grows in the absence of the agent; wherein a potential agent is identified as an agent that inhibits bacterial growth when there is a decrease in the growth of the bacterial culture in the presence of the agent relative to in its absence.

13. The method of claim 12, further comprising:
(e) preparing a supplemental crystal containing the core RNA polymerase formed in the presence of the potential agent, wherein the crystal effectively diffracts X-rays for the determination of the atomic coordinates to a resolution of better than 5.0 Angstroms;
(f) determining the three-dimensional coordinates of the supplemental crystal with molecular replacement analysis; and
(g) selecting a second generation agent by performing rational drug design with the three-dimensional coordinates determined for the supplemental crystal, wherein said selecting is performed in conjunction with computer modeling.

14. The method of claim 13, further comprising:
(h) contacting the second generation agent with a eukaryotic cell; and
(i) measuring the amount of proliferation of the eukaryotic cell under conditions in which the eukaryotic cell proliferates in the absence of the agent; wherein an agent is identified as an agent for inhibiting bacterial growth when there is no change in the proliferation of the eukaryotic cell in the presence of the agent relative to in its absence; and wherein the agent identified inhibits bacterial growth but not eukaryotic proliferation.

15. A method of identifying an agent for use as an inhibitor of bacterial RNA polymerase comprising:
(a) selecting a potential agent by performing rational drug design with the set of atomic coordinates in Table 2, wherein said selecting is performed in conjunction with computer modeling;
(b) contacting the potential agent with a bacterial RNA polymerase; and
(c) measuring the activity of the bacterial RNA polymerase; wherein a potential agent is identified as an agent that inhibits bacterial RNA polymerase when there is a decrease in the activity of the bacterial RNA polymerase in the presence of the agent relative to in its absence.

16. The method of claim 15, further comprising:
(d) preparing a crystal containing a bacterial RNA polymerase formed in the presence of the potential agent, wherein the crystal effectively diffracts X-rays for the determination of the atomic coordinates to a resolution of better than 5.0 Angstroms;
(e) determining the three-dimensional coordinates of the crystal with molecular replacement analysis; and
(f) selecting a second generation agent by performing rational drug design with the three-dimensional coordinates determined for the crystal, wherein said selecting is performed in conjunction with computer modeling.

17. The method of claim 16, further comprising:
(g) contacting the second generation agent with a eukaryotic RNA polymerase; and
(h) measuring the activity of the eukaryotic RNA polymerase; wherein an agent is identified as an agent for use as an inhibitor of bacterial RNA polymerase when there is no change in the activity of the eukaryotic RNA polymerase in the presence of the agent relative to in its absence; and wherein the agent identified inhibits bacterial but not eukaryotic RNA polymerase.

18. A method of identifying an agent that inhibits bacterial growth comprising:
(a) selecting a potential agent by performing rational drug design with the set of atomic coordinates in Table 2, wherein said selecting is performed in conjunction with computer modeling;
(b) contacting the potential agent with a bacterial culture; and
(c) measuring the growth of the bacterial culture under conditions in which the bacterial culture grows in the absence of the agent; wherein a potential agent is identified as an agent that inhibits bacterial growth when there is a decrease in the growth of the bacterial culture in the presence of the agent relative to in its absence.

19. The method of claim 18 further comprising:
(d) preparing a crystal containing a bacterial RNA polymerase formed in the presence of the potential agent, wherein the crystal effectively diffracts X-rays for the determination of the atomic coordinates to a resolution of better than 5.0 Angstroms;
(e) determining the three-dimensional coordinates of the crystal with molecular replacement analysis; and
(f) selecting a second generation agent by performing rational drug design with the three-dimensional coordinates determined for the crystal, wherein said selecting is performed in conjunction with computer modeling.

20. The method of claim 19, further comprising:
(g) contacting the second generation agent with a eukaryotic cell; and
(h) measuring the amount of proliferation of the eukaryotic cell under conditions in which the eukaryotic cell proliferates in the absence of the agent; wherein an agent is identified as an agent for inhibiting bacterial growth when there is no change in the proliferation of the eukaryotic cell in the presence of the agent relative to in its absence; and wherein the agent identified inhibits bacterial growth but not eukaryotic proliferation.

21. A method of obtaining a crystal of an inhibitor bound to a core bacterial RNA polymerase comprising
(a) growing the core bacterial RNA polymerase crystal in a buffered solution containing 40-45% saturated ammonium sulfate, wherein a crystal forms; and
(b) soaking the crystal in 2 M (NH₄)₂SO₄, with the inhibitor; where a crystal of the inhibitor bound to the core bacterial RNA polymerase is formed.

22. The method of claim 21 wherein the inhibitor is rifampicin.

23. The method of claim 21 wherein said growing is performed by a method selected from the group consisting of batch crystallization, vapor diffusion, and microdialysis.

24. (Twice Amended) A method of identifying a compound that is predicted to inhibit bacterial RNA polymerase comprising:
(a) defining the structure of rifampicin bound to the core RNA polymerase (Rif-RNAP) or a portion of the Rif-RNAP by the atomic coordinates in Table 2; wherein the portion of the Rif-RNAP comprises sufficient structural information to perform step (b); and
(b) identifying a compound that is predicted to inhibit bacterial RNA polymerase; wherein said identifying is performed using the structure defined in step (a).

25. The method of claim 24, further comprising:
(c) contacting the compound with a bacterial RNA polymerase; and
(d) measuring the activity of the bacterial RNA polymerase; wherein the compound is identified as an agent that inhibits bacterial RNA polymerase when there is a decrease in the activity of the bacterial RNA polymerase in the presence of the compound relative to in its absence.

26. (Amended) The method of claim 25, further comprising:
(e) contacting the compound with a eukaryotic RNA polymerase; and
(f) measuring the activity of the eukaryotic RNA polymerase; wherein the compound is identified as an agent for use as an inhibitor of bacterial RNA polymerase when there is no change in the activity of the eukaryotic RNA polymerase in the presence of the compound relative to in its absence; and wherein the compound identified inhibits bacterial but not eukaryotic RNA polymerase.

27. (Amended) A method of identifying a compound that is predicted to inhibit bacterial growth comprising:
(a) defining the structure of rifampicin bound to the core RNA polymerase (Rif-RNAP) or a portion of the Rif-RNAP by the atomic coordinates in Table 2; wherein the portion of the Rif-RNAP comprises sufficient structural information to perform step (b); and
(b) identifying a compound that is predicted to inhibit bacterial growth; wherein said identifying is performed using the structure defined in step (a).

28. The method of claim 27, further comprising:
(c) contacting the compound with a bacterial culture; and
(d) measuring the growth of the bacterial culture under conditions in which the bacterial culture grows in the absence of the compound; wherein the compound is identified as an agent that inhibits bacterial growth when there is a decrease in the growth of the bacterial culture in the presence of the compound relative to in its absence.

29. (Amended) The method of claim 28, further comprising:
(e) contacting the compound with a eukaryotic cell; and
(f) measuring the amount of proliferation of the eukaryotic cell under conditions in which the eukaryotic cell proliferates in the absence of the compound; wherein the compound is identified as an agent for inhibiting bacterial growth when there is no change in the proliferation of the eukaryotic cell in the presence of the compound relative to in its absence; and wherein the compound identified inhibits bacterial growth but not eukaryotic proliferation.

30. A computer having within its memory a representation of rifampicin bound to the core RNA polymerase (Rif-RNAP) or a portion of the Rif-RNAP comprising:
(a) a machine-readable data storage medium comprising a data storage material encoded with machine-readable data, wherein said data comprises structural coordinates from Table 2;
(b) a working memory for storing instructions for processing said machine-readable data;
(c) a central processing unit coupled to said working memory and to said machine-readable data storage medium for processing said machine readable data into a three-dimensional representation of the Rif-RNAP complex or a portion of the Rif-RNAP; and
(d) a display coupled to said central-processing unit for displaying said three-dimensional representation.
